(19) United States
(12) Patent Application Publication
(10) Pub. No.: US 2026/0285876 A1
(43) Pub. Date: Sep. 24, 2026

(54) MACROCYCLIC COMPOUNDS AS USP1 INHIBITORS

(71) Applicant: GUANGDONG NEWOPP BIOPHARMACEUTICALS CO., LTD., Guangdong (CN)

(72) Inventors: Yongshuai CHAI, Guangdong (CN); Xinsuo ZHAO, Guangdong (CN); Zhaoyin WANG, Richmond (CA); Lintong LI, San Diego, CA (US); Nanxin LI, San Diego, CA (US)

(21) Appl. No.: 19/145,452

(22) PCT Filed: Jan. 3, 2024
(86) PCT No.: PCT/CN2024/070267

§ 371 Date: Jul. 2, 2025

(30) Foreign Application Priority Data
Jan. 3, 2023 (US) 63475935
Apr. 13, 2023 (US) 63577269
Jul. 24, 2023 (US) 63628449

(51) Int. Cl. C07D 498/16; A61K 31/519; A61K 31/53; A61P 35/00; C07D 498/22; C07D 515/22

(52) U.S. Cl. CPC C07D 498/16; A61K 31/519; A61K 31/53; A61P 35/00; C07D 498/22; C07D 515/22

(57) ABSTRACT

Disclosed a novel series of macrocyclic compounds of Formula I as USP1 inhibitors and their use for the treatment of diseases associated with USP1 enzyme.

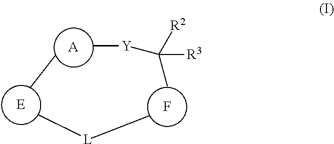

### TECHNICAL FIELD

The invention is directed to a series of novel macrocyclic compounds as USP1 inhibitors useful for the treatment of diseases or conditions, such as cancers, especially cancers with BRCA mutations or HR deficiencies. Pharmaceutical compositions and methods of use are also included.

### BACKGROUND

This invention relates to macrocyclic compounds, or their pharmaceutically acceptable salts, pharmaceutical acceptable prodrugs, pharmaceutical compositions containing them, and their medical uses.

The compounds of this invention have activity as ubiquitin-specific protease 1 (USP1) inhibitors and are useful in the treatment or alleviation of diseases associated with USP1 enzyme, such as cancers and other disorders. In particular, due to the important role of USP1 in regulating DNA damage response (DDR) pathways, inhibition of USP1 has the potential to be a treatment for cancers with BRCA (BReast CAncer) gene mutations or deficiency for homologous recombination (HR).

Ubiquitin is a small 76-amino-acids protein which can be conjugated to specific target proteins via polyubiquitination and monoubiquitination. Deubiquitination is a reversible process which involves a family of deubiquitinating enzymes (DUBs) that catalyze the removal of ubiquitin moieties from ubiquitinated substrates. Ubiquitylation plays a regulatory role in protein interactions, localization and function, thereby affecting cellular processes, including gene expression, DNA damage signaling and DNA repair, cell cycle progression, apoptosis and cell motility, and so forth.

As a member of the DUBs, USP1 plays an important role in the regulation of DNA repair processes. USP1 forms a heterodimeric complex with UAF1 (USP1-associated factor 1), which is required for deubiquitinase activity. The mono-ubiquitinated PCNA (proliferating cell nuclear antigen) is a critical substrate of USP1-UAF1. During DNA replication, PCNA monoubiquitylation facilitates the recruitment of specific TLS (translesion synthesis) polymerases, which can bypass the DNA lesions. Inhibition of USP1 leads to replication fork destabilization due to persistent TLS polymerase loading and accumulation of toxic mono-ubiquitinated PCNA. Replication fork instability can cause double strand breaks (DSBs), which require BRCA-mediated HR repair. Therefore, like Poly-(ADP)-ribose polymerase (PARP) inhibitors, USP1 inhibition is synthetic lethal with BRCA mutations or deficiency for HR. Another DNA repair-related process, Fanconi anemia (FA) pathway that repair DNA inter-strand crosslink (ICL) lesions, is also regulated by USP1/UAF1. The critical USP1 substrate in FA pathway is FANCD2 (Fanconi anemia group complementation group D2) and FANC1 (Fanconi anemia complementation group I). Besides these DNA repair-related functions, USP1 has been reported to deubiquitinate and stabilize inhibitors of DNA binding proteins to maintain stem-cell characteristics in osteosarcoma cells.

### DESCRIPTION

The invention encompasses a genus of compounds of Formula I as USP1 inhibitors or a pharmaceutically acceptable salt thereof,

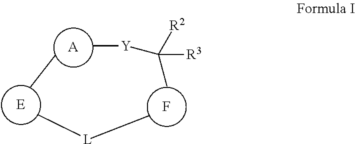

### Wherein

Ring A is selected from the following structures:

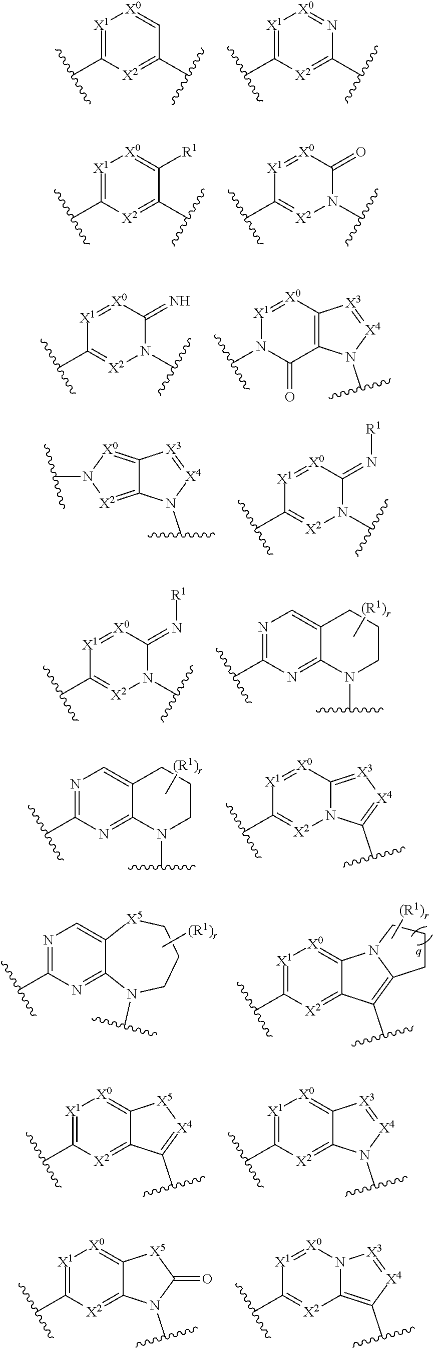

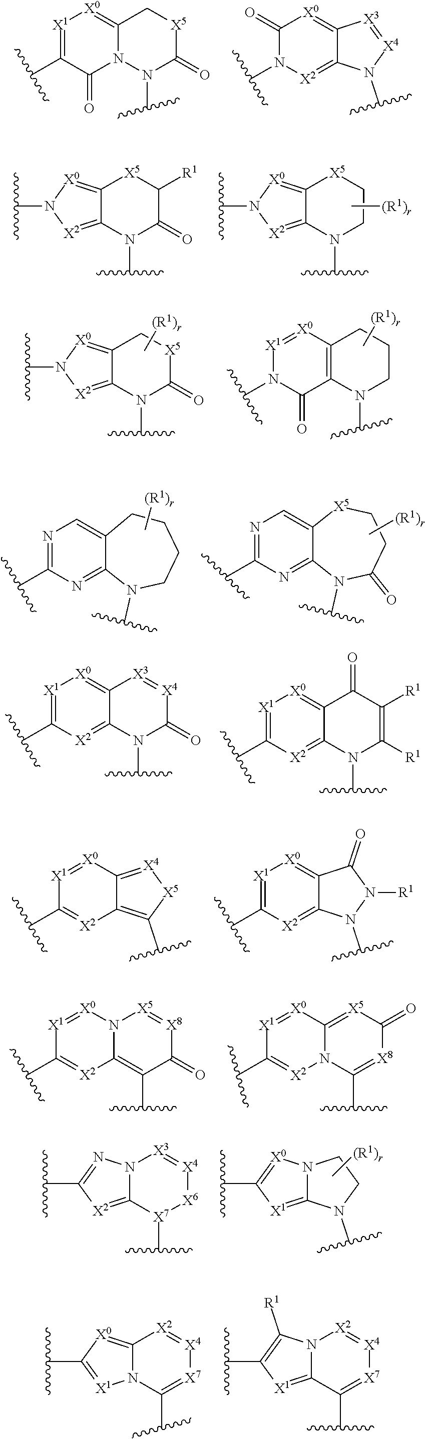

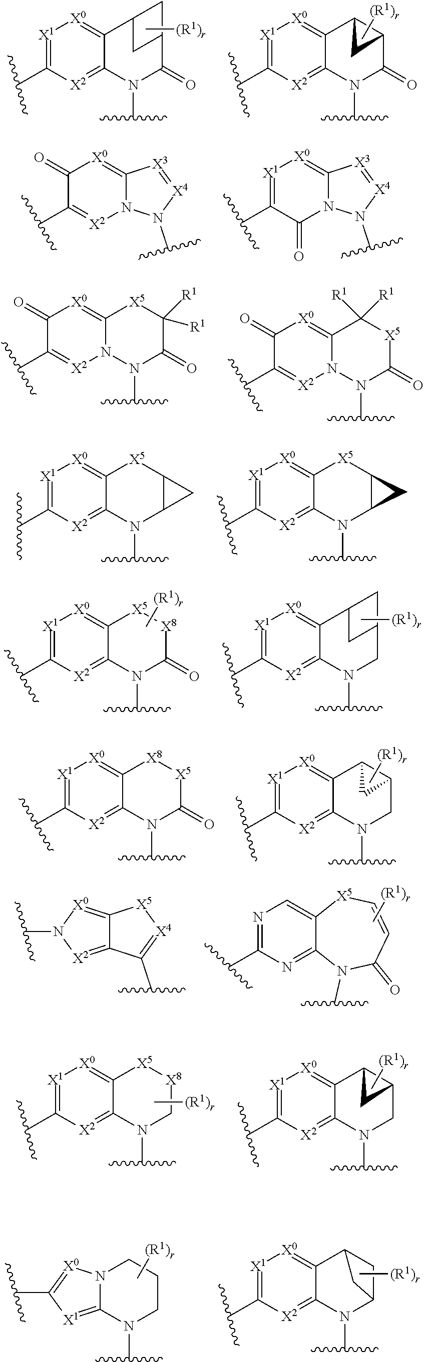

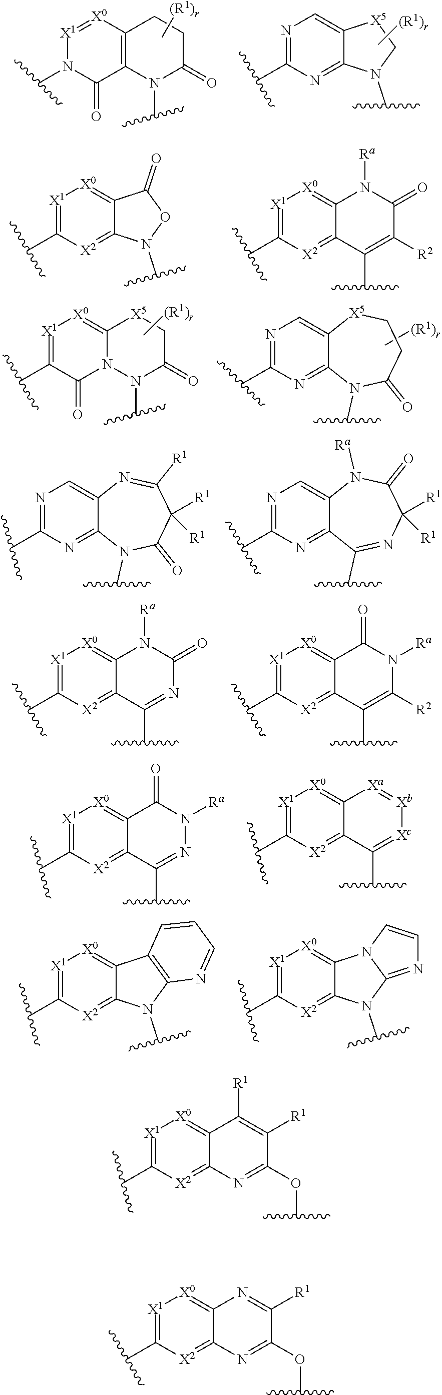

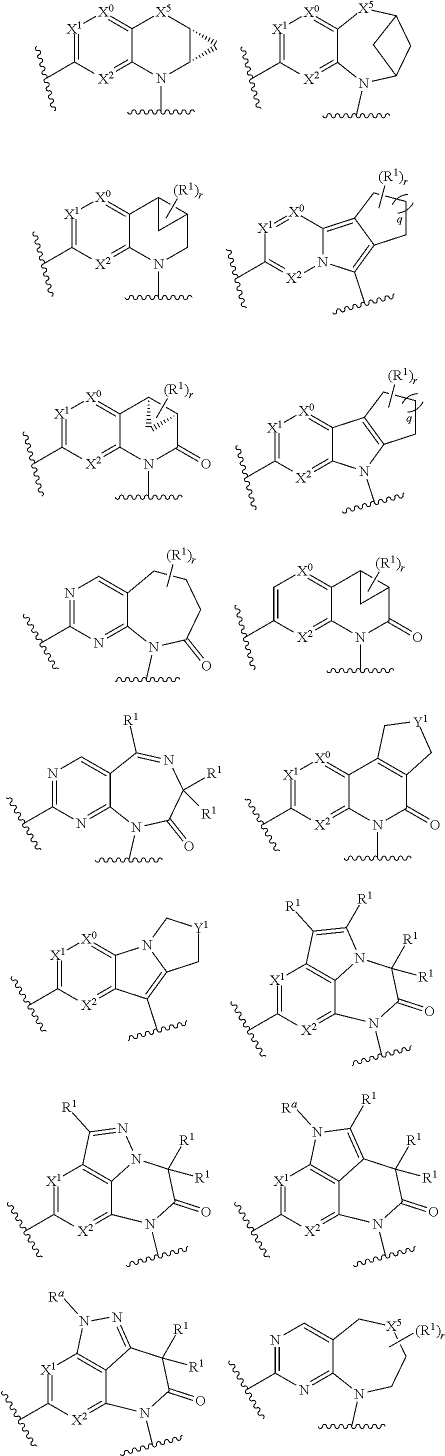

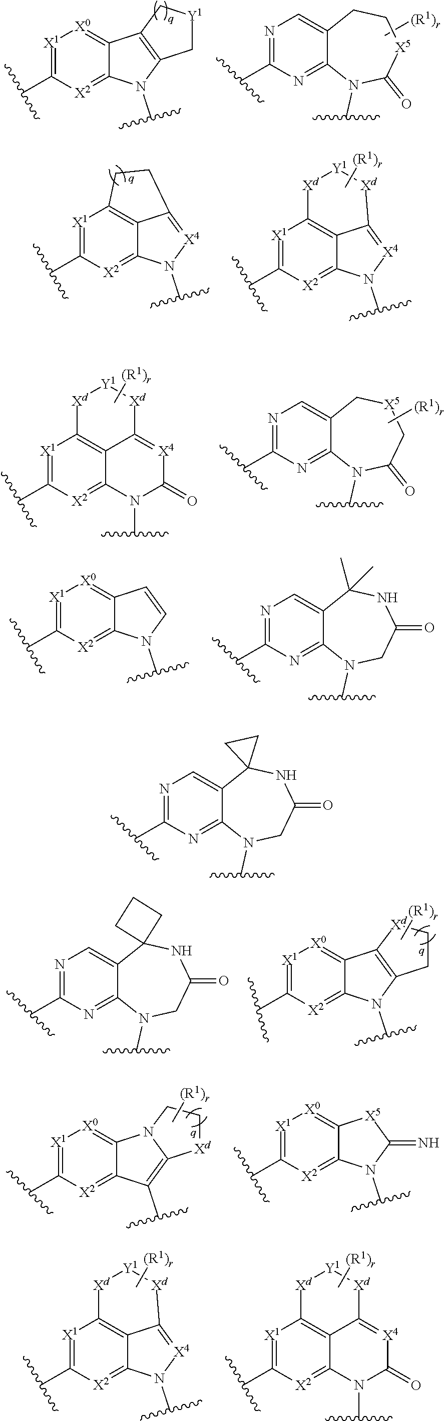

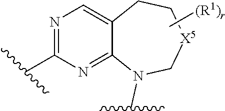

Y and Y¹ are independently a chemical bond or selected from NR¹, O, S, S(O)₂ or C(R¹)₂;
Each of X⁰, X¹, X², X³, X⁴, X^{a}, X^{b} and X^{c} is independently selected from N or CR¹;
X⁵, X⁸ is independently selected from C(R¹), C(R¹)₂, O, S or NR¹;
r and q are independently selected from 0, 1, 2, 3 or 4;
X⁶ is NR^{b}, C═O, CR^{a}R^{b};
X⁷ is N or CR¹;
X^{d} is independently a chemical bond or selected from CH₂, C(O), O, S, S(O)₂, O or NR^{d};
R¹ is independently selected from the group consisting of hydrogen, halogen, C₁-C₆ alkyl, C₁-C₆ halogenated alkyl, C₁-C₆ alkoxy, C₁₋₆ alkylthio, hydroxyl, nitro, formyl, —CN, —SF₅, —NR^{a}R^{b}, carboxyl, —C(O)R^{a}, —C(O)₂C₁-C₆ alkyl, —C(O)NR^{a}R^{b}, —S(O)₂R^{c}, —SO₂NR^{a}R^{b}, —P(O)Me₂ and —P(O)(OMe)₂;
Two R¹ on the same carbon atom or adjacent carbon atoms together with carbon atom(s) can form an oxo group or a three- to six-membered carbocyclic ring, four- to eight-membered bicyclic ring or five- to eight-membered spirocyclic ring which is optionally substituted with R^{c} or a three- to six-membered carbocyclic ring, four- to eight-membered bicyclic ring or five- to eight-membered spirocyclic ring which contains one, two or three heteroatom(s) such as S, O or NR^{d};
R^{a} and R^{b} are independently hydrogen, CH₂CH₂OH, CH₂CMe₂OH, C₁-C₆ alkanol, C₁-C₆ halogenated alkanol, substituted/non-substituted C₁-C₁₀ alkyl, substituted/non-substituted C₃-C₁₀ cycloalkyl, substituted/non-substituted C₂-C₁₀ alkenyl, substituted/non-substituted C₆-C₂₀ aryl, or substituted/non-substituted C₃-C₁₄ heteroaryl respectively; R^{a} and R^{b} can form 3-8 membered carbocyclic rings or 4-8 membered heterocyclic rings, which contain sulfur, oxygen, NH or NR^{d};
R^{c} represents C₁-C₁₀ alkyl, C₃-C₁₀ cycloalkyl, C₆-C₂₀ aryl, or C₃-C₁₄ heteroaryl; R^{c} can be substituted by one or more radical groups selected from halogen, hydroxyl, amino, nitro, cyano, formyl, carboxyl, alkoxy, —CF₃ and —SF₅;
R^{d} is selected from the group consisting of hydrogen, C₁₋₆alkyl, C₃₋₆cyclolkyl, C₃₋₆fluorocycloalkyl, C₁₋₆fluoroalkyl, C₀₋₆alkylene-aryl, C₀₋₆alkylene-heteroaryl, C(O)C₁₋₆alkyl, C(O)NR^{a}R^{b}, C(O)aryl, S(O)₂C₁₋₆alkyl, S(O)₂C₃₋₆cycloalkyl, S(O)₂ary, S(O)₂NR^{a}R^{b};
R^{e} is independently selected from the group consisting of hydrogen, halogen, C₁-C₆ alkyl, C₁-C₆ halogenated alkyl, C₁-C₆ alkoxy, nitro, formyl, —CN, —SF₅, NH₂, C₁-C₁₀ alkyl, C₃-C₁₀ cycloalkyl, C₆-C₂₀ aryl, or C₃-C₁₄ heteroaryl;
Each of R² and R³ is independently selected from the group consisting of H, NR^{a}R^{b}, C₁-C₆ alkoxy, C₁₋₆ alkylthio, halogen, CN, C₁₋₆alkyl, C₃-C₆cyclolkyl, C₃-C₆fluorocycloalkyl, C₁-C₆fluoroalkyl, C₂-C₆ alkenyl, C₂-C₆ alkynyl, wherein the alkyl, alkenyl, alkynyl, and cycloalkyl are optionally substituted with one or more substituents selected from halogen, C₁-C₆alkyl, C₁-C₆halogenated alkyl, C₃-C₁₀cycloalkyl, C₃-C₁₀ cycloalkoxy, C₁-C₆alkoxy, hydroxyl, nitro, formyl, —CN, —SF₅, —NR^{a}R^{b}, carboxyl, —C(O)R^{a}, —C(O)₂C₁-C₆ alkyl, —C(O)NR^{a}R^{b}, —S(O)₂R^{c}, —S(O)₂NR^{a}R^{b}, —P(O)Me₂ and —P(O)(OMe)₂; R² and R³, together with the carbon atom to which they are both attached to, complete a three- to six-membered carbocyclic ring, four- to ten-membered bicyclic ring or five- to ten-membered spirocyclic ring which is optionally substituted with R^{d} or a three- to six-membered carbocyclic ring, four- to eight-membered bicyclic ring or five- to eight-membered spirocyclic ring which contains one, two or three heteroatom(s) such as S, O or NR^{d};
Ring E represents C₆-C₂₀ aromatic ring or C₁-C₂₀ heteroaromatic ring substituted by one or more groups selected from halogen, C₁-C₆ alkyl, C₁-C₆ alkanol, C₁-C₆ halogenated alkyl, C₃-C₁₀ cycloalkyl, C₃-C₁₀ cycloalkoxy, C₁-C₆ alkoxy, hydroxyl, nitro, formyl, —CN, —SF₅, —NR^{a}R^{b}, carboxyl, —C(O)R^{a}, —C(O)₂C₁-C₆ alkyl, —C(O)NR^{a}R^{b}, —S(O)₂R^{c}, —S(O)₂NR^{a}R^{b}, —P(O)Me₂ and —P(O)(OMe)₂;

Preferably

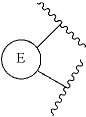

is selected from the following structures:

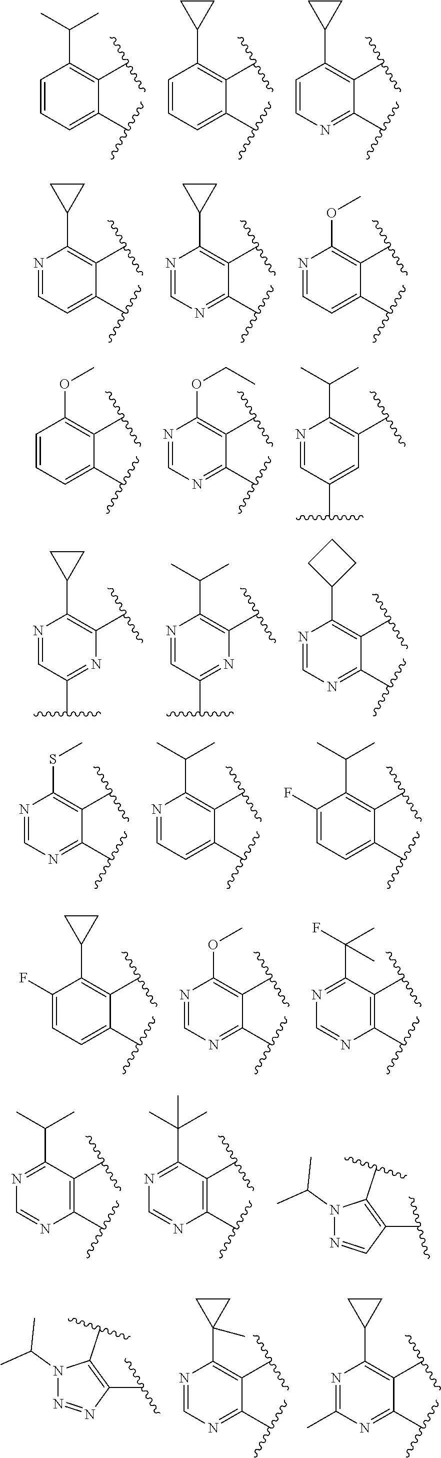

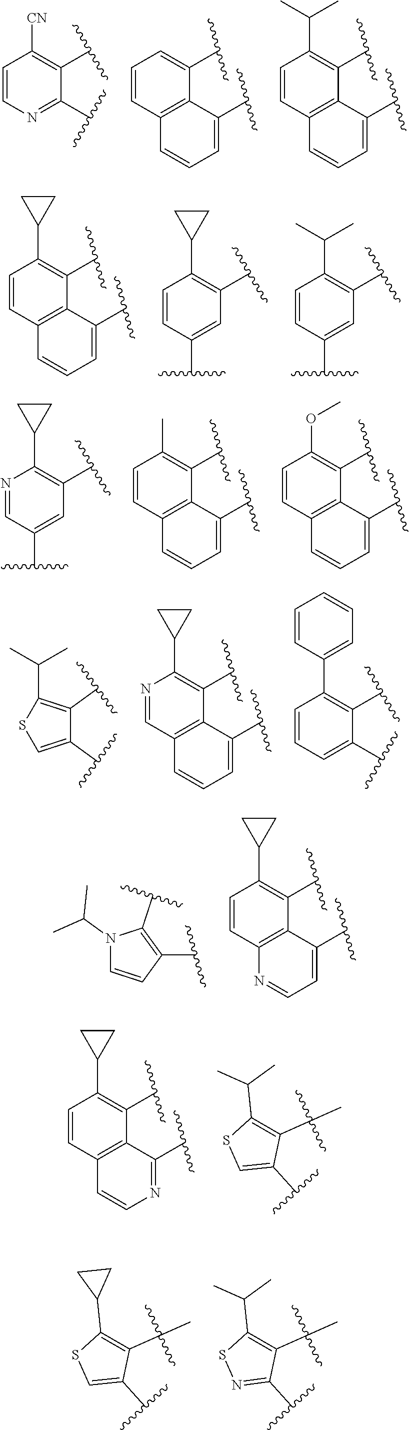

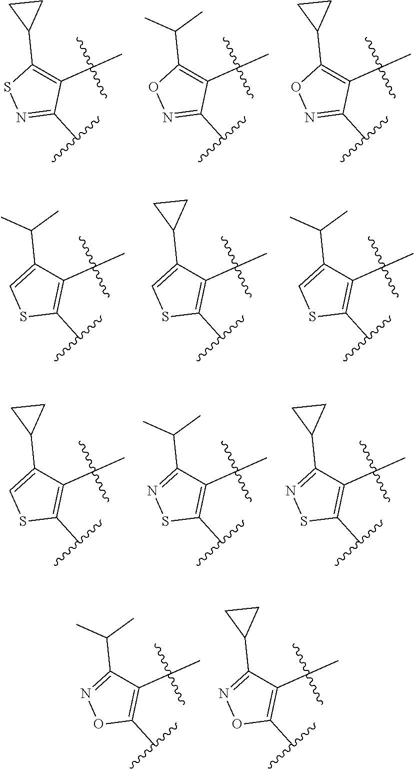

Ring F represents C₁-C₆aromatic ring, C₁-C₂₀heteroaromatic ring, C₃-C₁₄carbocyclic ring, C₄-C₁₄bicarbocyclic ring, C₅-C₁₄spirocarbocyclic ring, C₄-C₁₄heterocyclic ring, C₄-C₁₄heterobicyclic ring, C₅-C₁₄heterotricyclic ring, C₅-C₁₄heterospirocyclic ring with 0 to 5 of O, N or S atoms independently; Ring F is substituted with one or more R⁴;

Preferably

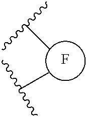

is selected from the following structures:

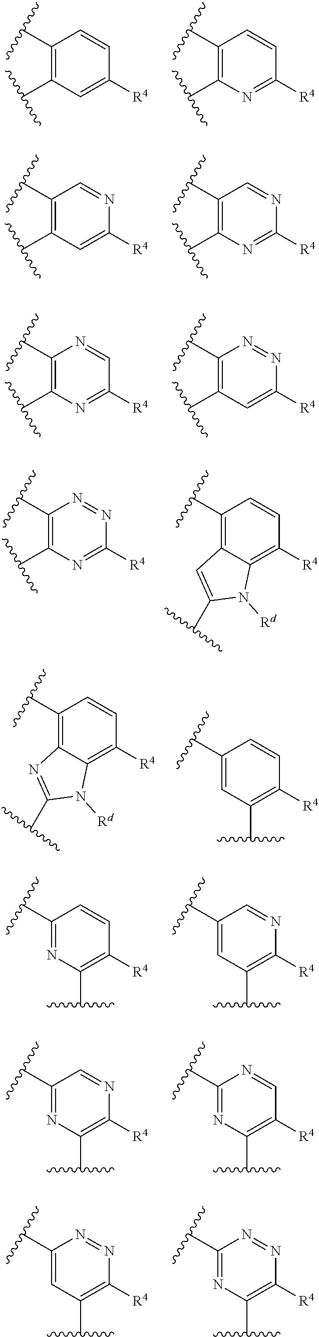

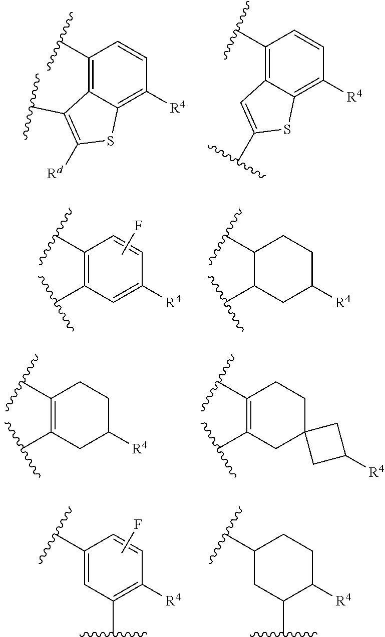

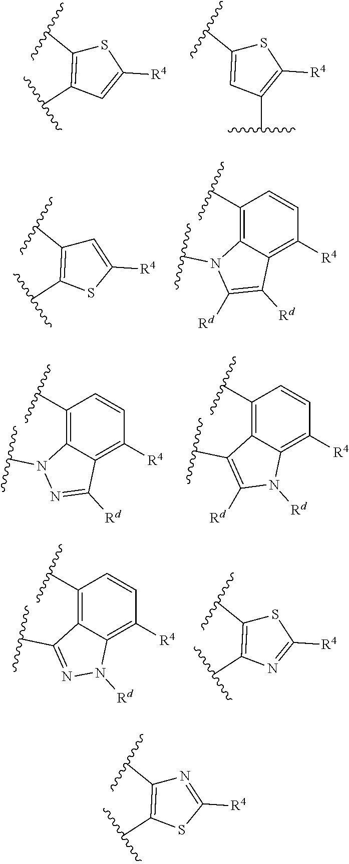

R⁴ represents optionally substituted C₁-C₆alkyl, optionally substituted C₂-C₆alkenyl, optionally substituted C₂-C₆alkynyl, optionally substituted C₁-C₄alkoxy, C₁-C₆haloalkyl, C₁-C₄haloalkoxy, C₁-C₆hydroxyalkyl, —CN, halogen, —S(O)₂NH₂, —C(O)R^{a}, —C(O)₂R^{a}, —NR^{b}R^{b}, —NR^{a}C(O)R^{b}, —NR^{a}C(O)NR^{a}R^{a}, —C(O)NR^{a}R^{b}, —S(O)₂NR^{a}R^{b}, —NR^{a}S(O)₂R^{b}, optionally C₆-C₁₀ aryl and C₁-C₁₀heteroaryl; R⁴ can be substituted by one or more groups selected from halogen, C₁-C₆alkyl, C₃-C₆cycloalkyl, C₃-C₆ halogenated cycloalkyl, C₁-C₆halogenated alkyl, C₁-C₆alkoxy, hydroxyl, amino, nitro, formyl, —CN, —SF₅, —NR^{a}R^{b}, carboxyl, —C(O)R^{a}, —C(O)₂C₁-C₆ alkyl, —C(O)NR^{a}R^{b}, —S(O)₂R^{c}, —S(O)₂NR^{a}R^{b}, —P(O)Me₂ and —P(O)(OMe)₂; preferably, R⁴ is selected from the following structures:

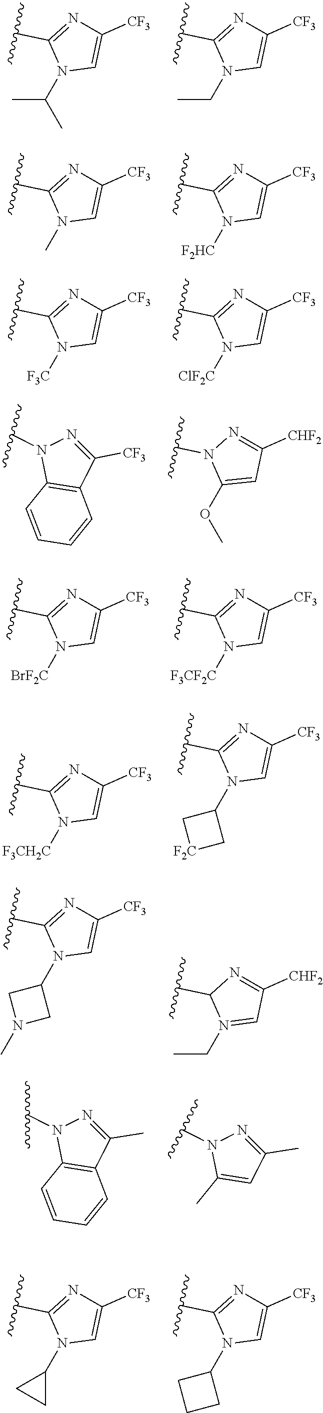

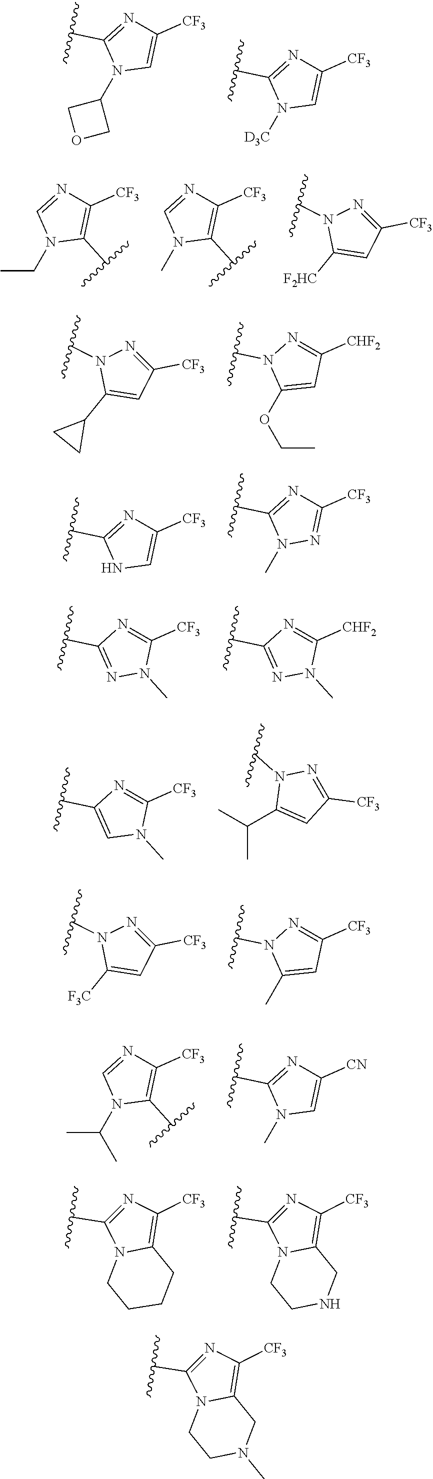

L is selected from —(CH₂)ₚ—, —O(CH₂)ₚ—, —(CH₂)ₚO—, —S(O)ᵥ(CH₂)ₚ—, —S(O)₂(NR⁵)(CH₂)ₚ—, —(CH₂)ₚS(O)ᵥ—, —(CH₂)ₚS(O)₂(NR⁵)—, —NR⁵(CH₂)ₚ—, —(CH₂)ₚNR⁵—, —(CH₂)ₚO(CH₂)ᵣ—, —(CH₂)ₚNR⁵(CH₂)ᵣ—, —(CH₂)ₚS(O)ᵥ(CH₂)ᵣ—, —(CH₂)ₚS(O)₂(NR⁵)(CH₂)ᵣ—, —(CH₂)ₚS(CH₂)ᵣ—, —O(CH₂)ₚO—, —NR⁵(CH₂)ₚNR⁶, —S(O)ᵥ(CH₂)ₚS(O)ᵥ—, —O(CH₂)ₚS(O)ᵥ—, —S(O)ᵥ(CH₂)ₚO—, —O(CH₂)ₚNR⁵—, —NR⁵(CH₂)ₚO—, —S(O)ᵥ(CH₂)ₚNR⁵—, —NR⁵(CH₂)ₚS(O)ᵥ—, —O(CH₂)ₚSO(NR⁵)—, —SO(NR⁵)(CH₂)ₚO—, —SO(NR⁵)(CH₂)ₚNR⁶—, —NR⁵(CH₂)ₚSO(NR⁶)—, —(CH₂)ₚCF₂(CH₂)ᵣ—, —(CH₂)ₚCHF(CH₂)ᵣ—, —(CF₂)₄—, —(CF₂)₅—, —(CH₂)ₚC(O)—, —O(CH₂)ₚC(O)—, —(CH₂)ₚC(O)NR⁵—, —(CH₂)ₚC(S)NR⁵—, —(CH₂)ₚCH(CF₃)NR⁵—, —(CH₂)ₚNR⁵C(O)—, —(CH₂)ₚNR⁵CH(CF₃)—, —C(O)NR⁵(CH₂)ₚ—, —CH(CF₃)NR⁵(CH₂)ₚ—, —C(S)NR⁵(CH₂)ₚ—, —O(CH₂)ₚC(O)NR⁵—, —O(CH₂)ₚC(S)NR⁵—, —S(O)ᵥ(CH₂)ₚC(O)—, —O(CH₂)ₚC(O)NR⁵—, —NR⁵C(O)(CH₂)ₚC(O)NR⁶—, —C(O)NR⁵(CH₂)ₚC(O)NR⁶—, —C(O)NR⁵(CH₂)ₚNR⁶C(O)—, —NR⁵C(O)(CH₂)ₚNR⁶C(O)—, —S(O)ᵥ(CH₂)ₚO—, —O(CH₂)ₚS(O)ᵥ—, —S(O)ᵥ(CH₂)ₚ—, —(CH₂)ₚS(O)ᵥ—, —S(O)ᵥ(CH₂)ₚS(O)ᵥ—, —NR⁵(CH₂)ₚC(O)NR⁶—, —NR⁵C(O)(CH₂)ₚ—, —NR⁵CH(CF₃)(CH₂)ₚ—, —NR⁵(CH₂)ₚO—, —(CH₂)ᵣOC(O)(CH₂)ₚ—, —OC(O)(CH₂)ₚ—, —OC(O)(CH₂)ₚS(O)ᵥ—, —(CH₂)ₚCR^{aa}═CR^{bb}(CH₂)ᵣ—, —NR⁵(CH₂)ₚCR^{aa}=CR^{bb}(CH₂)ᵣ—, —NR⁵C(O)(CH₂)ₚCR^{aa}═CR^{bb} (CH₂)ᵣ—, —(CH₂)ₚCR^{aa}═CR^{bb}(CH₂)ᵣC(O)NR⁵—, —(CH₂)ₚNR⁵C(O)NR⁶(CH₂)ᵣ—, —(CH₂)ₚNR⁵C(S)NR⁶(CH₂)ᵣ—, —(CH₂)ₚNR⁵S(O)₂NR⁶(CH₂)ᵣ—, —(CH₂)ₚS(O)ᵥ(CH₂)ᵣ—, —(CH₂)ₚS(O)₂NR⁵(CH₂)ᵣ—, —(CH₂)ₚNR⁵(O)ᵥ(CH₂)ᵣ—, —(CH₂)ₚS(CH₂)ᵣ—, —(CH₂)ₚC≡C(CH₂)ᵣ—, —O(CH₂)ₚCR^{aa}═CR^{bb}(CH₂)ᵣ—, —O(CH₂)ₚC≡C(CH₂)ᵣ—, —(CH₂)ₚCR^{aa}═CR^{bb} (CH₂)ᵣO—, —(CH₂)ₚC≡C(CH₂)ᵣO—, —O(CH₂)ₚCR^{aa}═CR^{bb}(CH₂)ᵣO—, —O(CH₂)ₚC≡C(CH₂)ᵣO—, —S(O)ᵥ(CH₂)ₚCR^{aa}═CR^{bb}(CH₂)ᵣ—, S(O)ᵥ(CH₂)ₚC≡C(CH₂)ᵣ—, —(CH₂)ₚCR^{aa}═CR^{bb} (CH₂)ᵣS(O)ᵥ—, —(CH₂)ₚC≡C(CH₂)ᵣS(O)ᵥ—, —O(CH₂)ₚCR^{aa}═CR^{bb}(CH₂)ᵣS(O)ᵥ—, —O(CH₂)ₚC≡C(CH₂)ᵣS(O)ᵥ—, —S(O)ᵥ(CH₂)ₚCR^{aa}═CR^{bb}(CH₂)ᵣO—, S(O)ᵥ(CH₂)ₚC≡C(CH₂)ᵣO—, —C(O)NR⁵S(O)₂(CH₂)ₚ—, or —(CH₂)ₚS(O)₂NR⁶C(O)—; or L is L₁-L₂-L₃;
L₁ and L₃ are independently selected from —(CH₂)ₚ—, —O(CH₂)ₚ—, —S(CH₂)ₚ—, —NR⁵(CH₂)ₚ—, —(CH₂)ₚNR⁵—, —(CH₂)ₚO—, —(CH₂)ₚS—, —(CH₂)ₚC(O)—, —C(O)(CH₂)ₚ—, —(CH₂)ₚC(O)NR⁵—, —NR⁵C(O)(CH₂)ₚ—, —(CH₂)ₚCR^{aa}═CR^{bb}(CH₂)ᵣ—, —O(CH₂)ₚCR^{aa}═CR^{bb}(CH₂)ᵣ—, —(CH₂)ₚCR^{aa}═CR^{bb}(CH₂)ᵣO—, —S(CH₂)ₚCR^{aa}═CR^{bb}(CH₂)ᵣ—, —(CH₂)ₚCR^{aa}═CR^{bb} (CH₂)ᵣS—, —O(CH₂)ₚCR^{aa}═CR^{bb} (CH₂)ᵣS—, or —S(CH₂)ₚCR^{aa}═CR^{bb} (CH₂)ᵣO—;
L₂ is a C₂₋₆heterocyclic ring, an C₆₋₁₀aryl ring or a C₁₋₉heteroaryl ring;
CR^{aa}═CR^{bb} (olefinic double bond) in L, L₁ and L₃ includes both E and Z geometric isomers;
R^{aa} and R^{bb} are independently selected from H, halogen, C₁₋₆alkyl, C₁₋₆halogenated alkyl, C₃₋₆ cycloalkyl, C₃₋₆halogenated cycloalkyl;
p is 1, 2, 3, 4, 5 or 6; v is 0, 1 or 2;
R⁵ and R⁶ are independently selected from hydrogen, C₁₋₆alkyl, C₃₋₆cycloalkyl.

In another preferred embodiment,

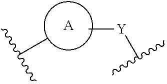

in Formula I is independently selected from the group consisting of:

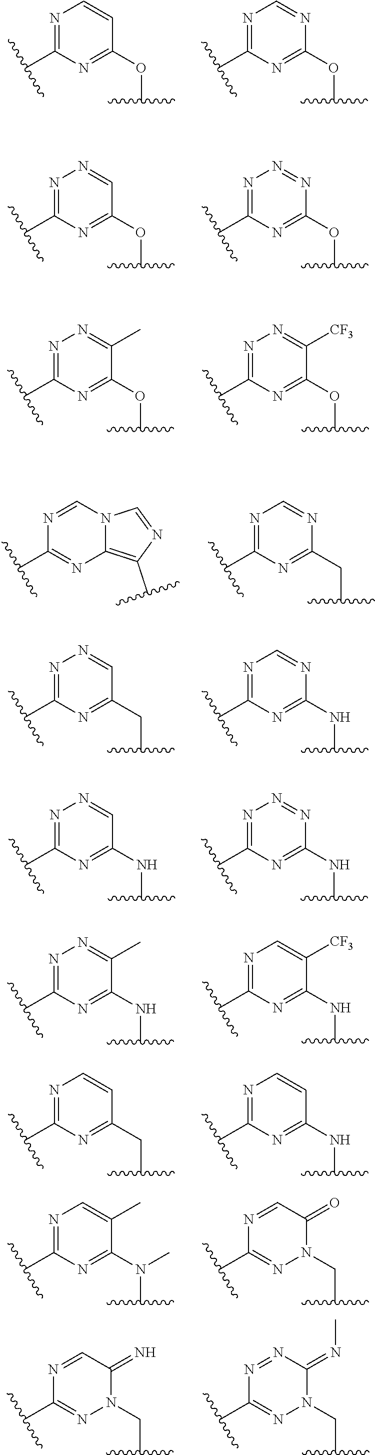

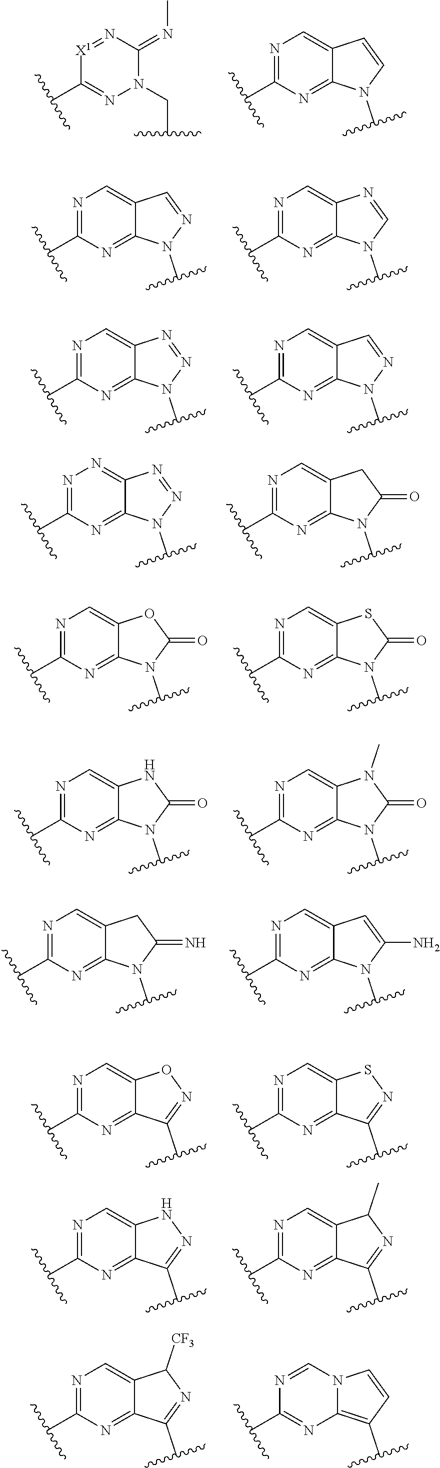

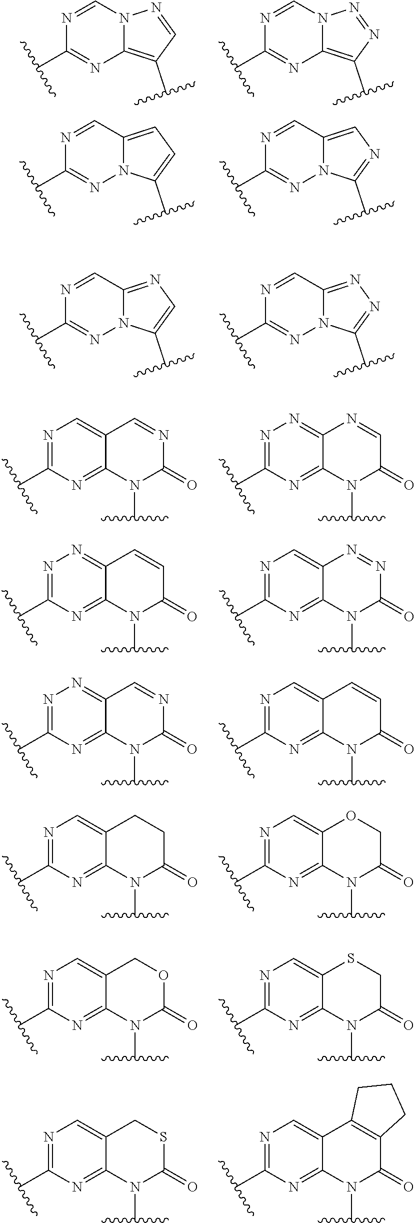

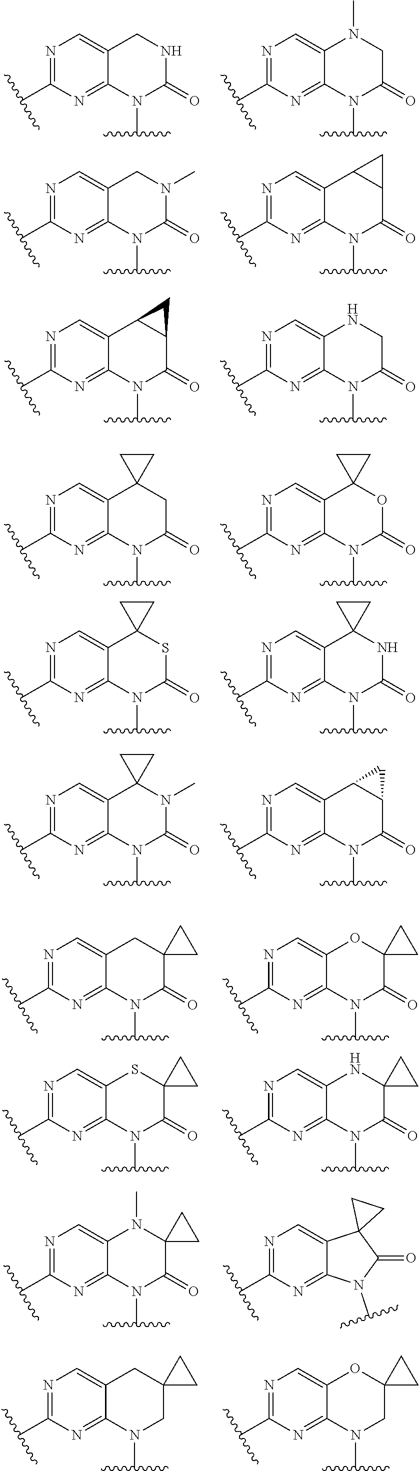

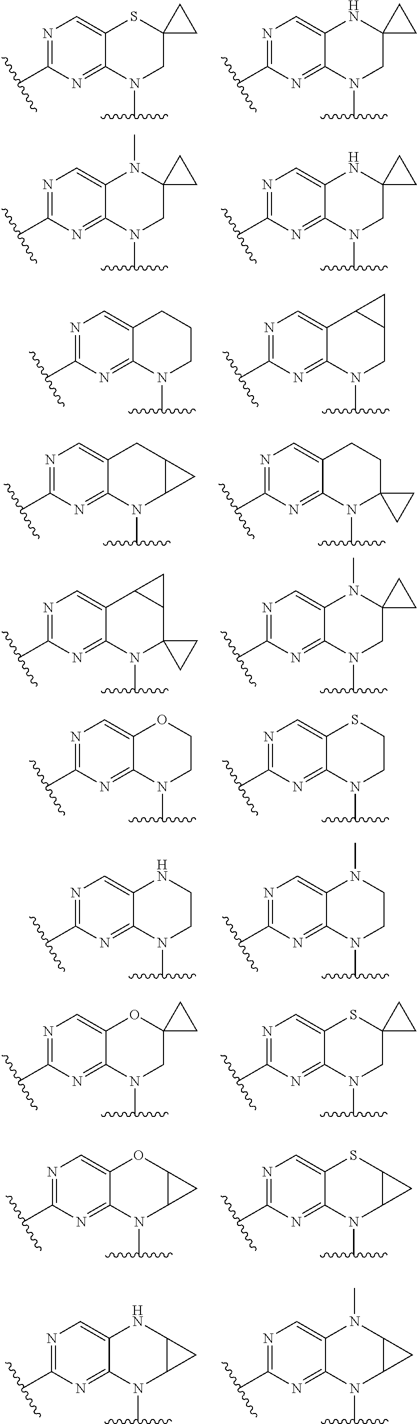

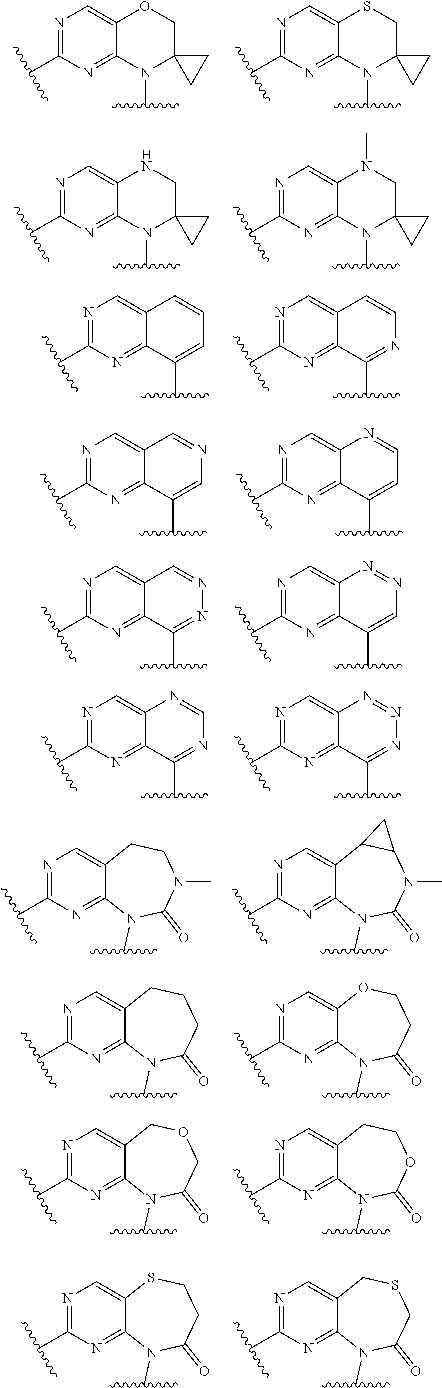

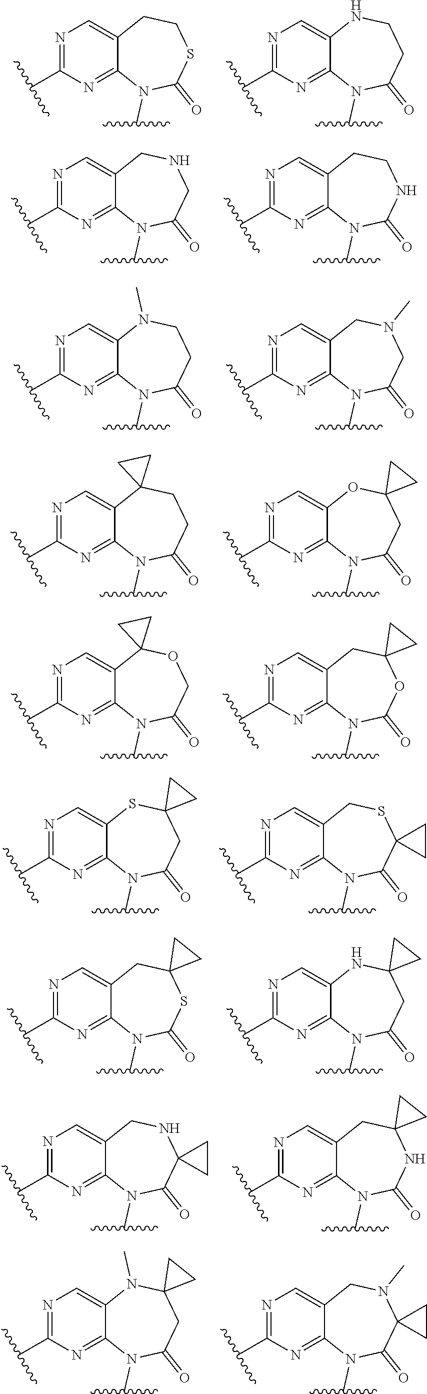

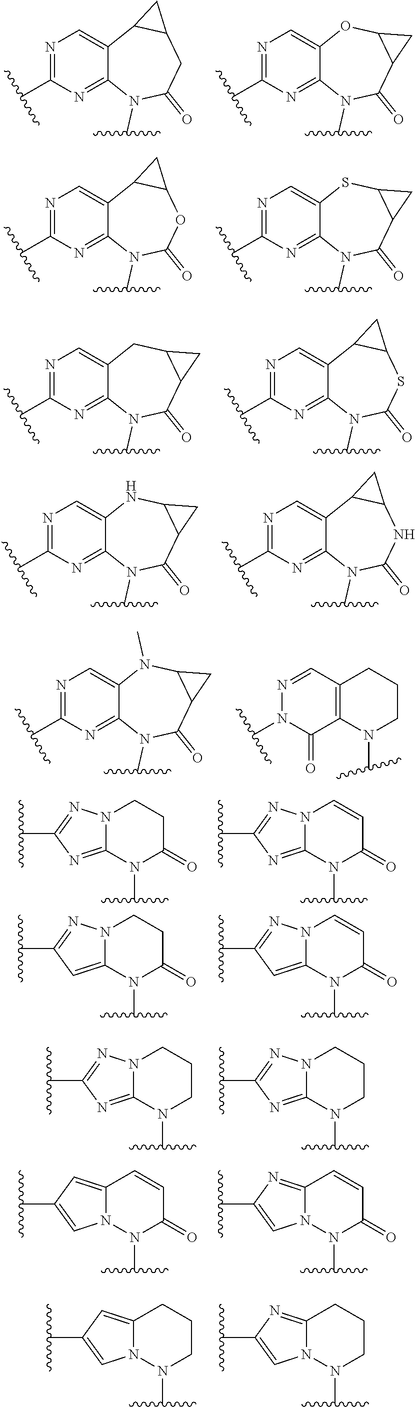

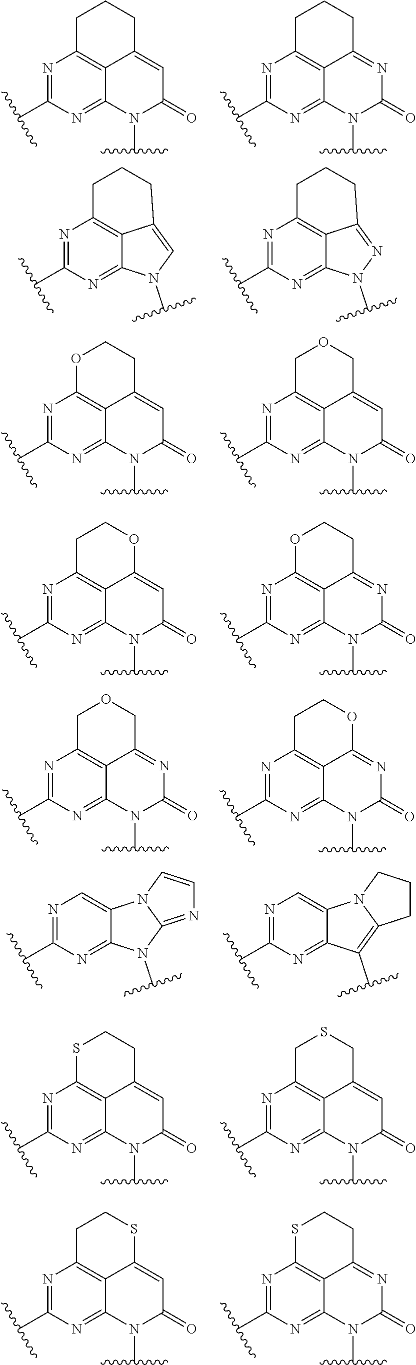

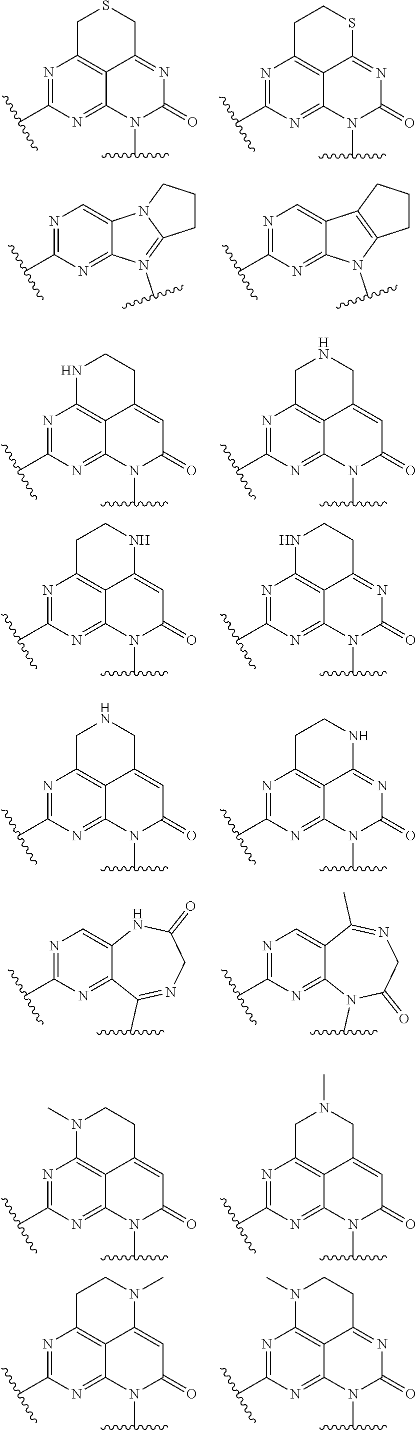

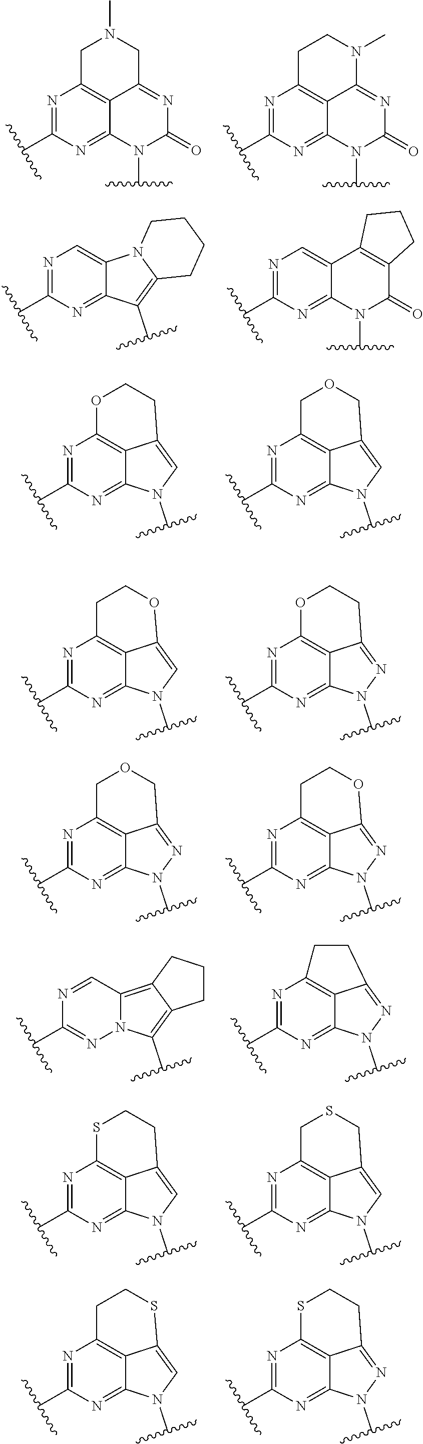

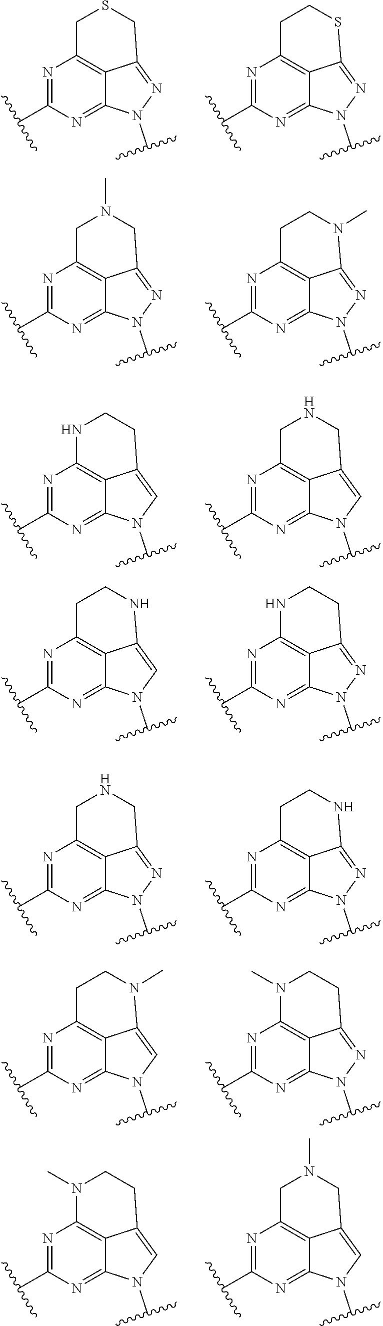

In another preferred embodiment, R⁴ is

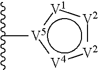

Each V¹, V², V³, and V⁴ are independently selected from C(R′), NR^{d}, O and S; V⁵ is C, or N; V¹, V², V³, V⁴ and V⁵ together form a 5-membered heteroaryl ring which is substituted with one, two or three R¹.

In one embodiment, the invention further provides a compound of Formula I or a pharmaceutically acceptable salt selected from Formula II to Formula XIX in the following table:

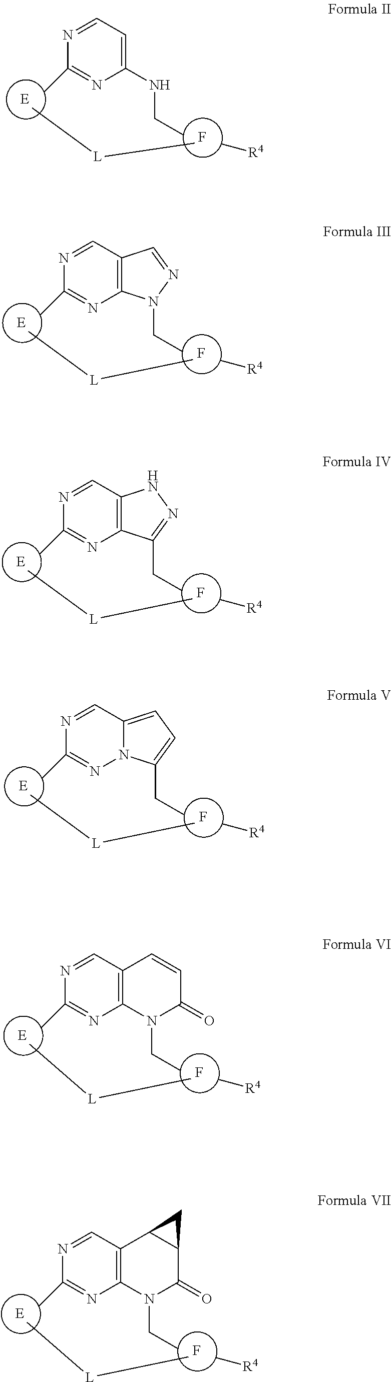

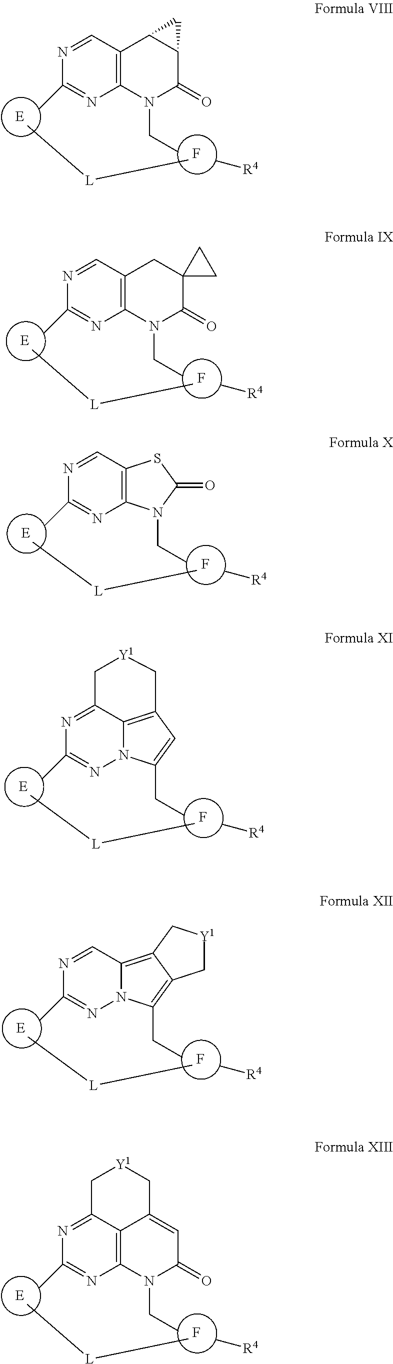

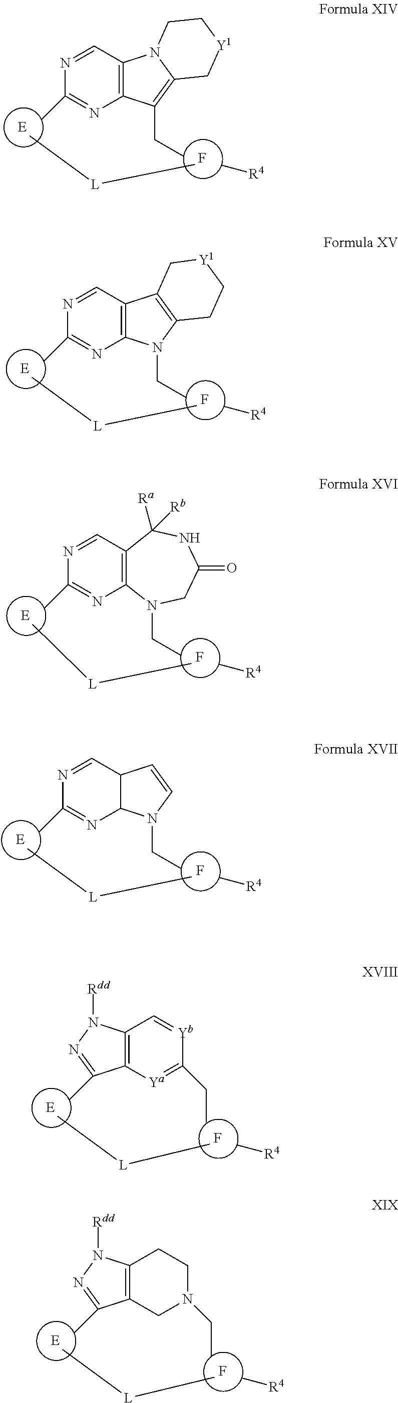

wherein ring E, ring F, L, Y¹, R^{a}, R^{b} and R⁴ are defined as in Formula I. Y^{a} and Y^{b} are independently N, CR^{ee}. R^{dd} is selected from H, C₁₋₆alkyl, halogenated C₁₋₆alkyl, C₃₋₆cycloalkyl. R^{ee} is selected from H, halogen, C₁₋₆alkyl, halogenated C₁₋₆alkyl, C₃₋₆cycloalkyl.

In some embodiments, a compound of Formula I to XIX is selected from the group consisting of the compounds below or a pharmaceutically acceptable salt thereof

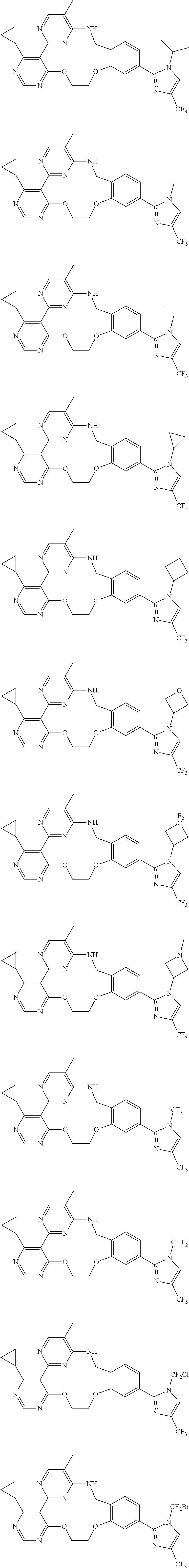

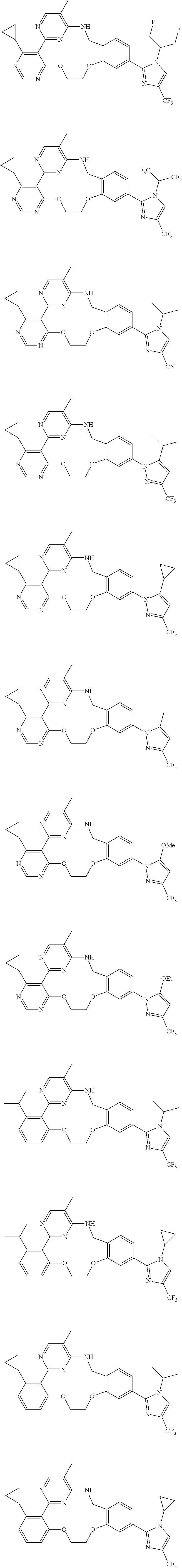

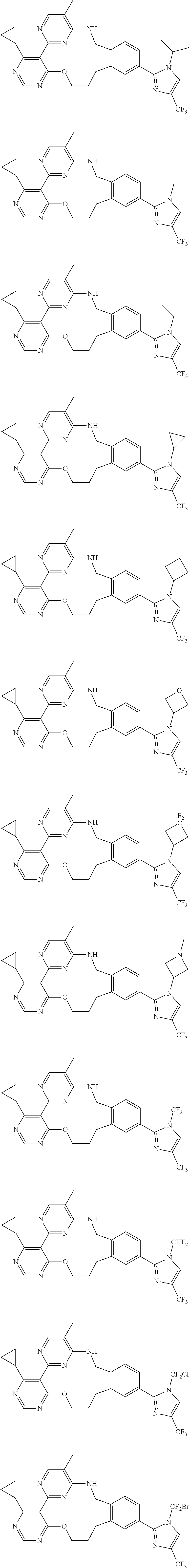

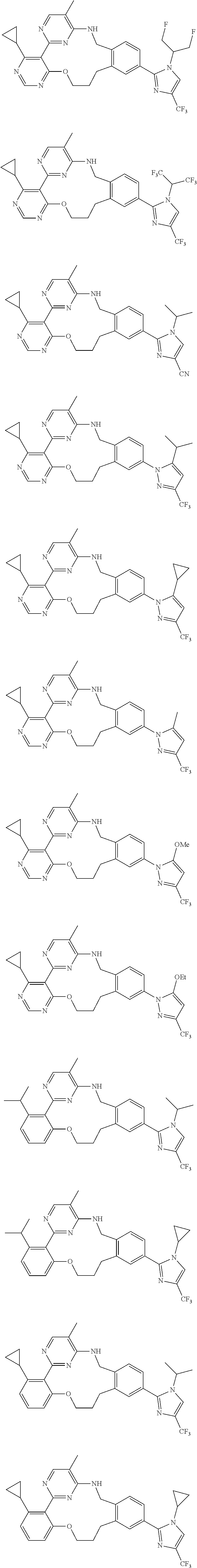

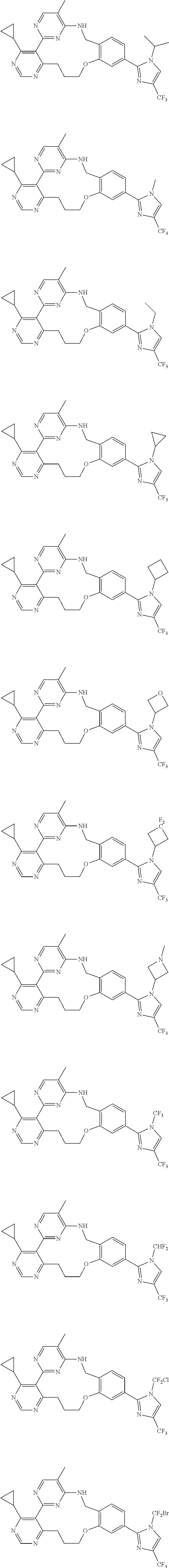

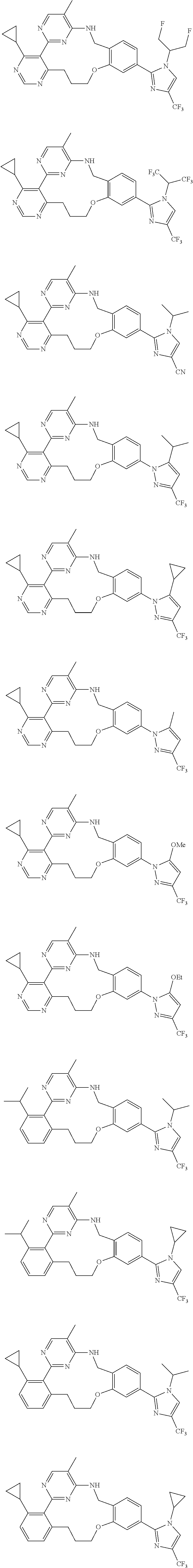

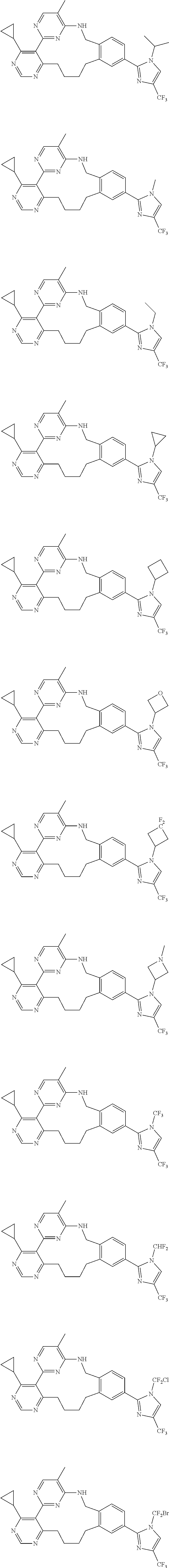

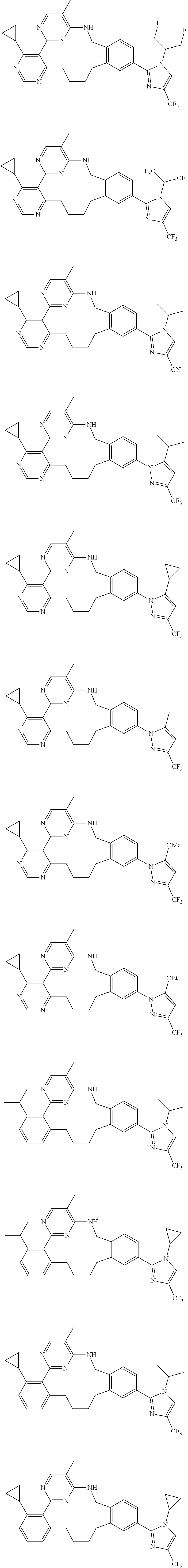

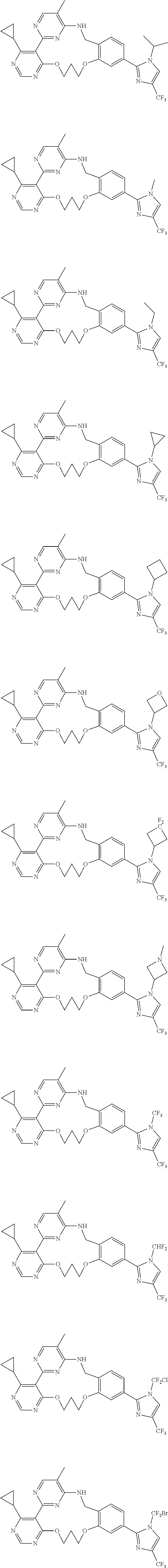

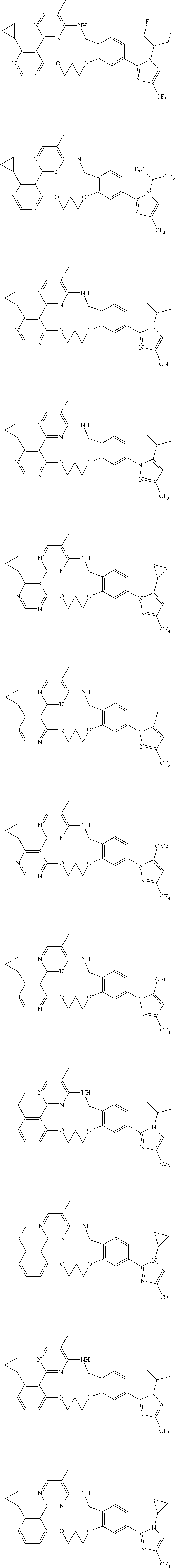

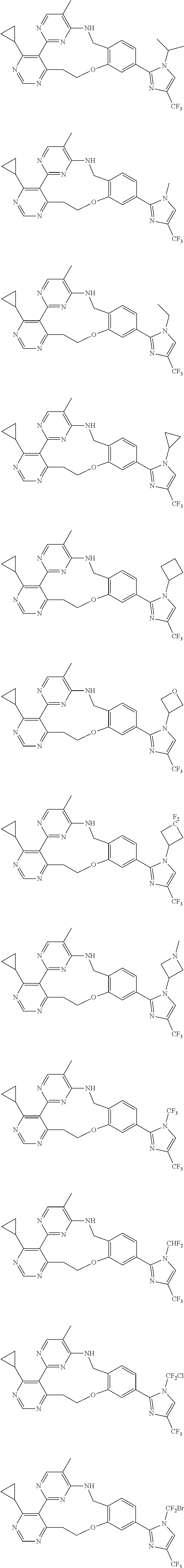

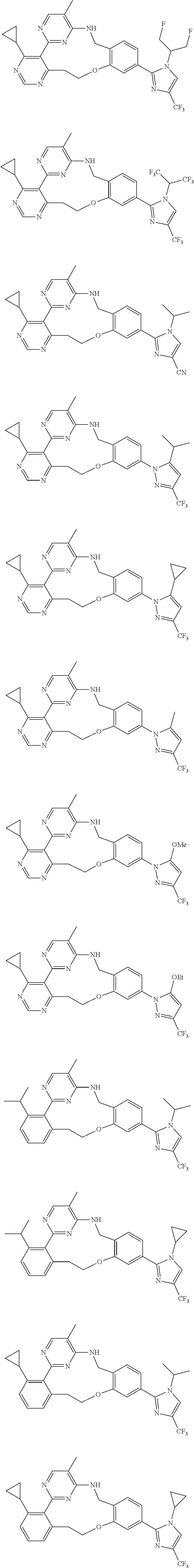

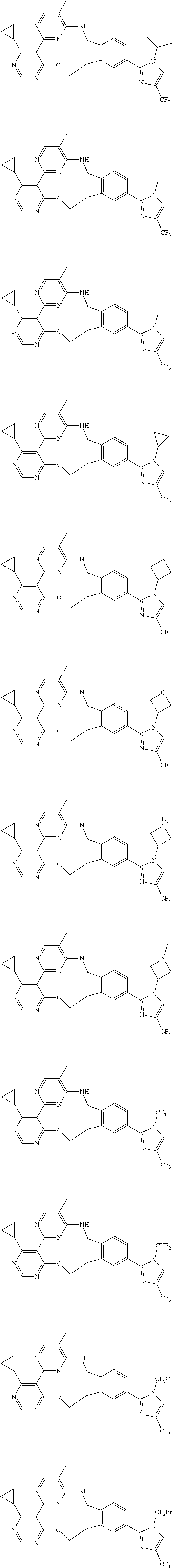

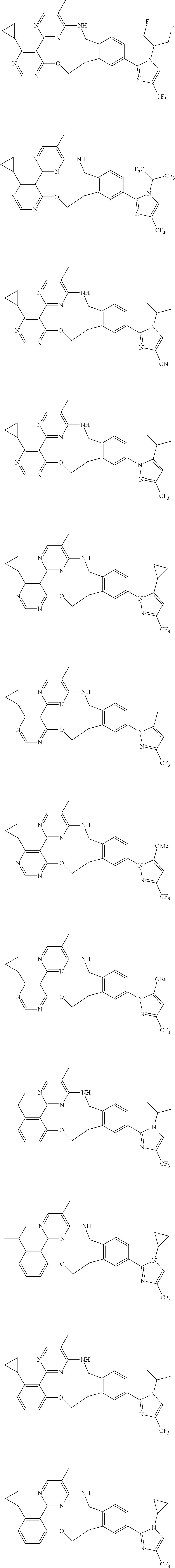

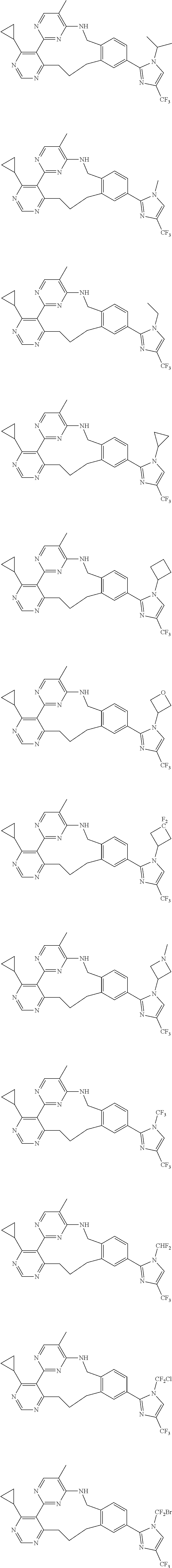

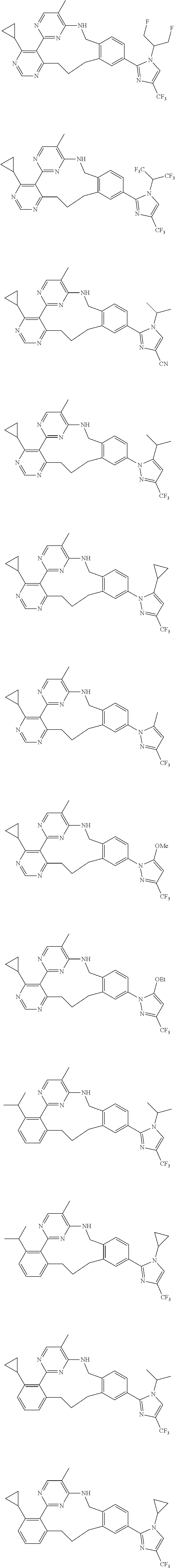

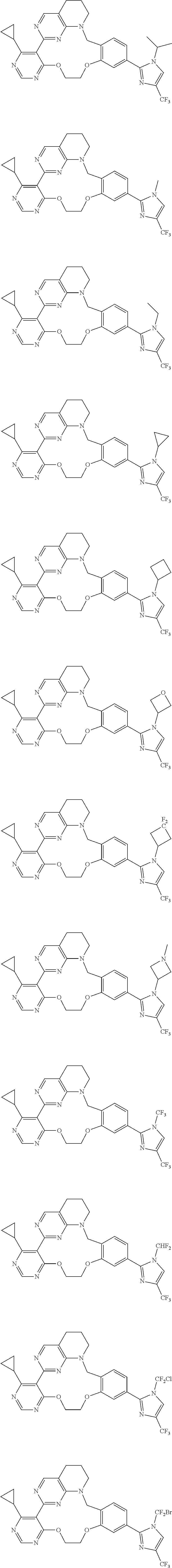

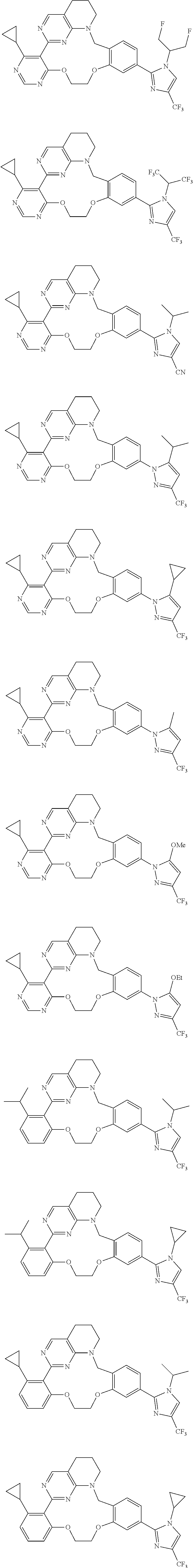

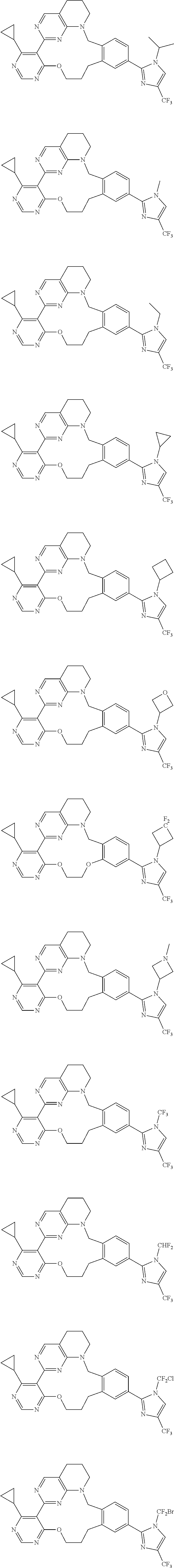

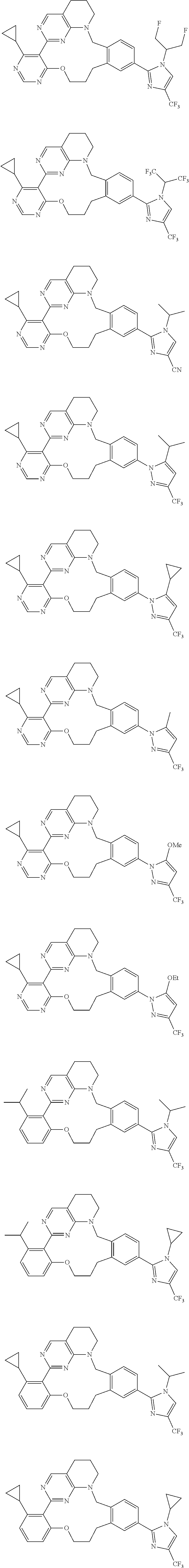

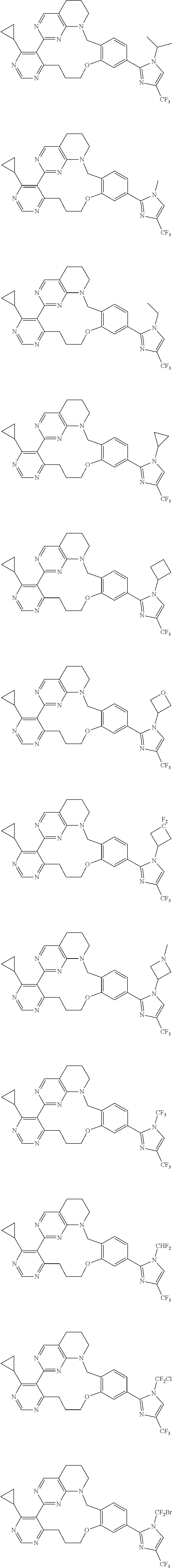

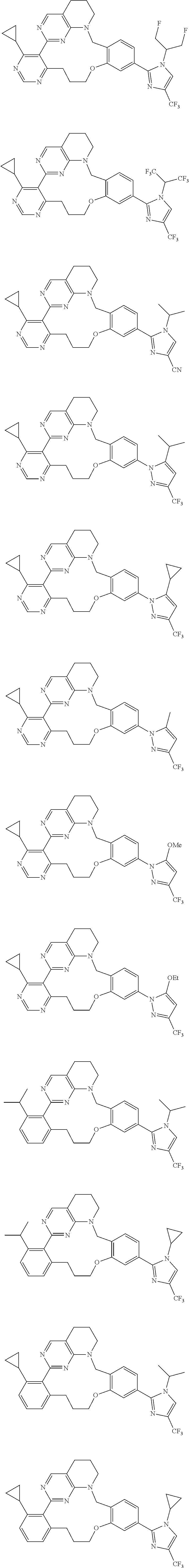

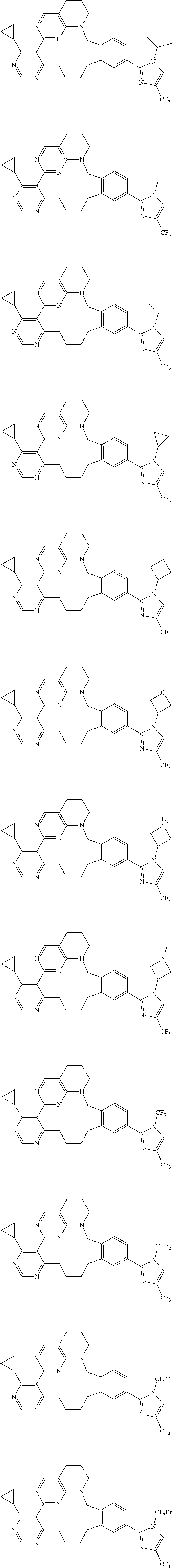

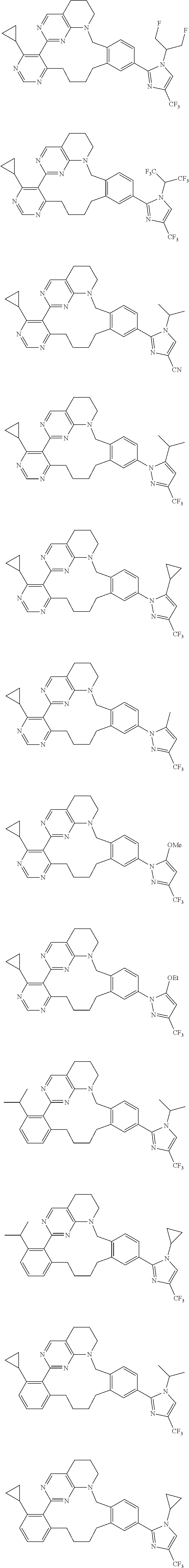

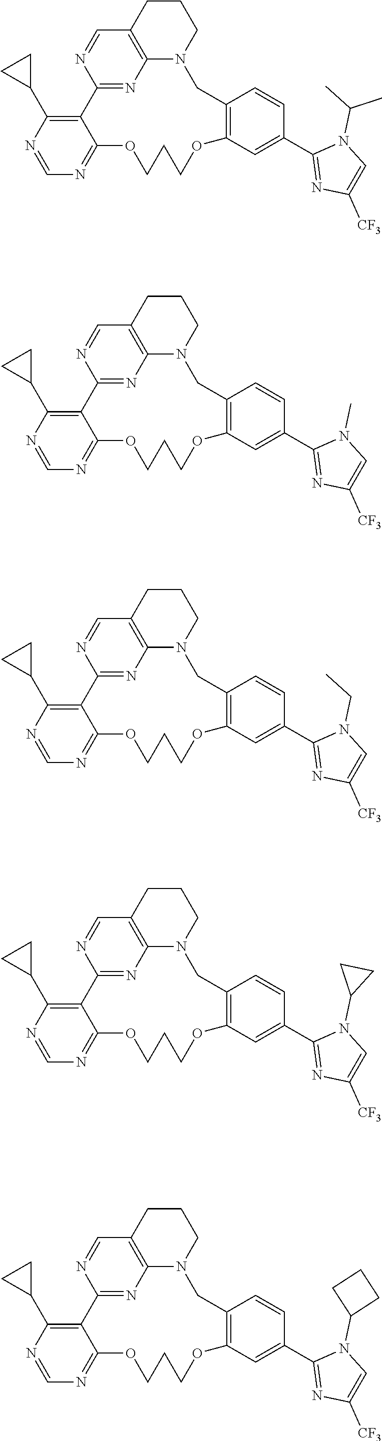 [SMILES: CC(C)n1cc(C(F)(F)F)nc1-c1ccc2c(c1)OCCCOc1ncnc(C3CC3)c1-c1ncc3c(n1)N(CCC3)C2 ; Cn1cc(C(F)(F)F)nc1-c1ccc2c(c1)OCCCOc1ncnc(C3CC3)c1-c1ncc3c(n1)N(CCC3)C2 ; CCn1cc(C(F)(F)F)nc1-c1ccc2c(c1)OCCCOc1ncnc(C3CC3)c1-c1ncc3c(n1)N(CCC3)C2 ; FC(F)(F)c1cn(C2CC2)c(-c2ccc3c(c2)OCCCOc2ncnc(C4CC4)c2-c2ncc4c(n2)N(CCC4)C3)n1 ; FC(F)(F)c1cn(C2CCC2)c(-c2ccc3c(c2)OCCCOc2ncnc(C4CC4)c2-c2ncc4c(n2)N(CCC4)C3)n1]

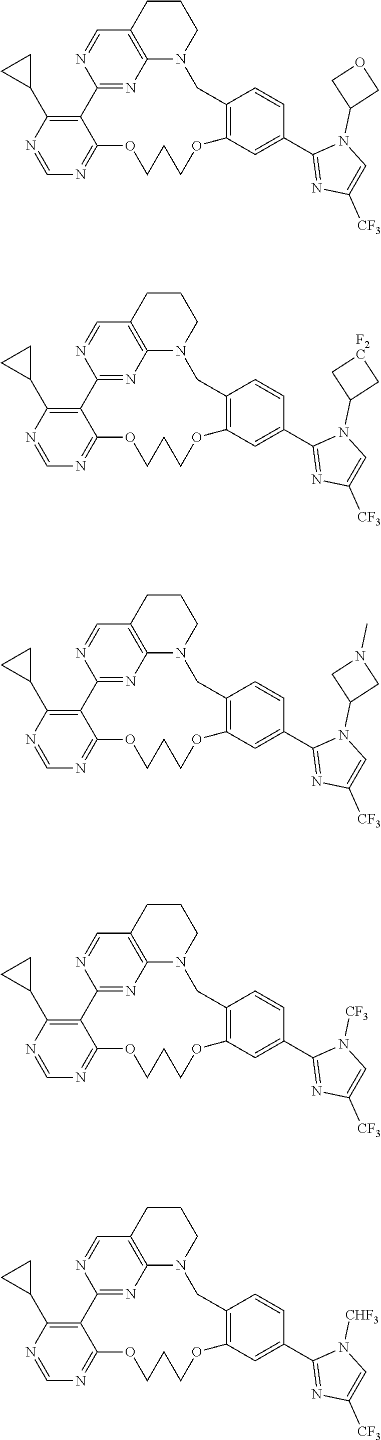

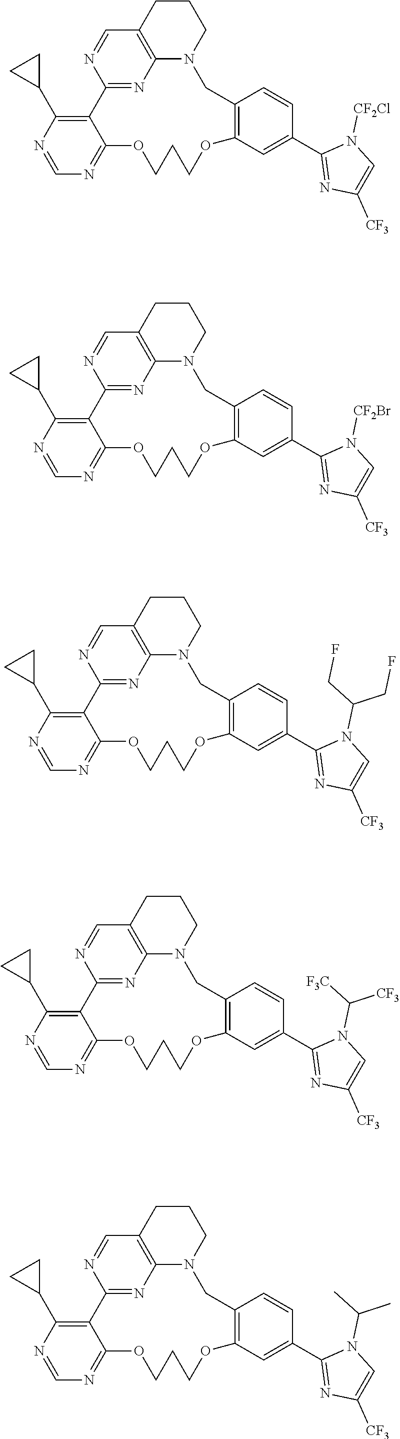

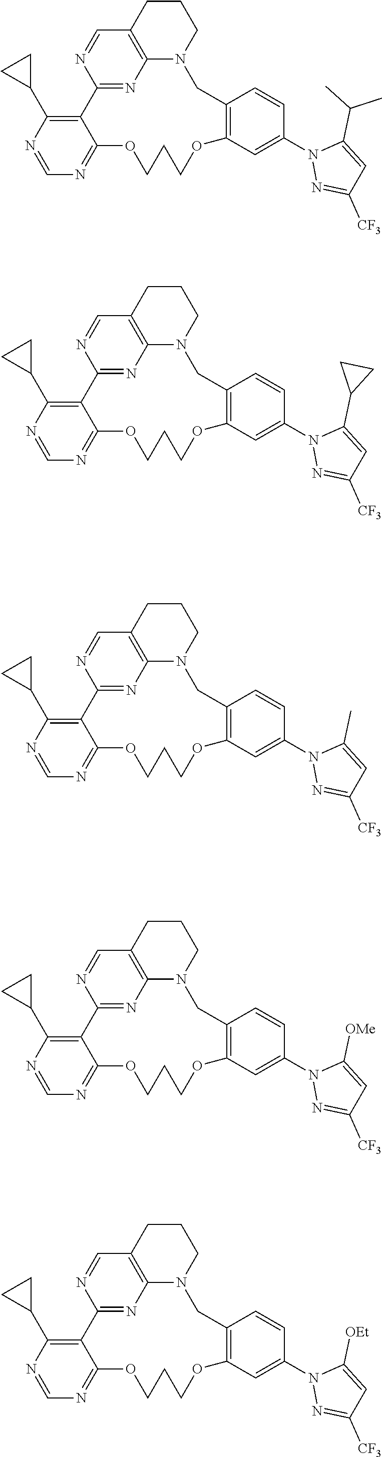 [SMILES: CC(C)c1cc(C(F)(F)F)nn1-c1ccc2c(c1)OCCCOc1ncnc(C3CC3)c1-c1ncc3c(n1)N(CCC3)C2 ; FC(F)(F)c1cc(C2CC2)n(-c2ccc3c(c2)OCCCOc2ncnc(C4CC4)c2-c2ncc4c(n2)N(CCC4)C3)n1 ; Cc1cc(C(F)(F)F)nn1-c1ccc2c(c1)OCCCOc1ncnc(C3CC3)c1-c1ncc3c(n1)N(CCC3)C2 ; COc1cc(C(F)(F)F)nn1-c1ccc2c(c1)OCCCOc1ncnc(C3CC3)c1-c1ncc3c(n1)N(CCC3)C2 ; CCOc1cc(C(F)(F)F)nn1-c1ccc2c(c1)OCCCOc1ncnc(C3CC3)c1-c1ncc3c(n1)N(CCC3)C2]

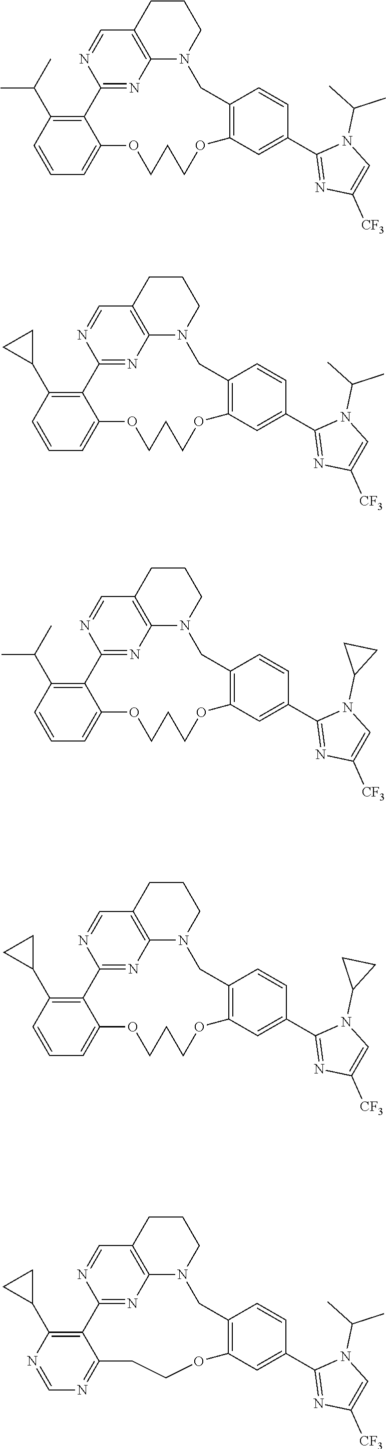

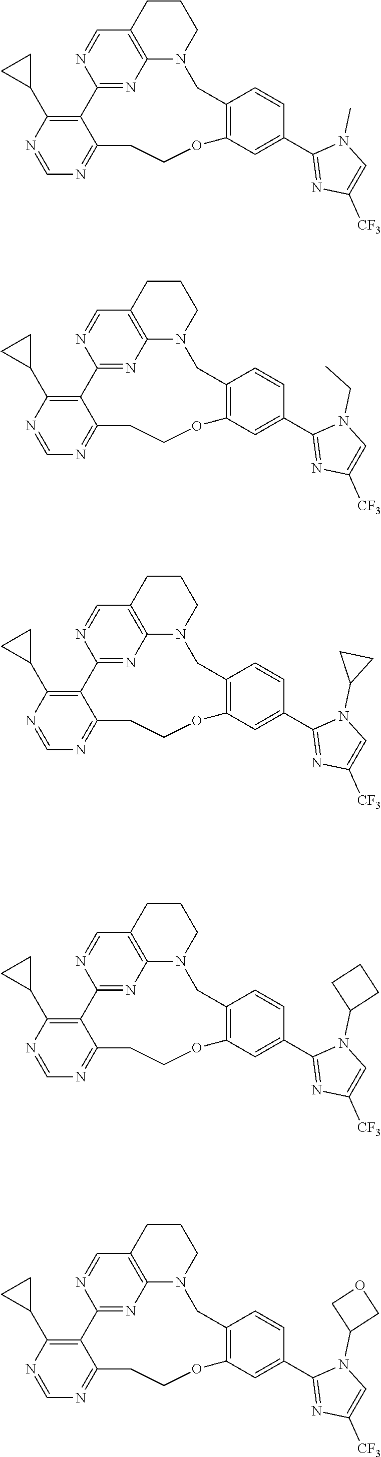 [SMILES: Cn1cc(C(F)(F)F)nc1-c1ccc2c(c1)OCCc1ncnc(C3CC3)c1-c1ncc3c(n1)N(CCC3)C2 ; CCn1cc(C(F)(F)F)nc1-c1ccc2c(c1)OCCc1ncnc(C3CC3)c1-c1ncc3c(n1)N(CCC3)C2 ; FC(F)(F)c1cn(C2CC2)c(-c2ccc3c(c2)OCCc2ncnc(C4CC4)c2-c2ncc4c(n2)N(CCC4)C3)n1 ; FC(F)(F)c1cn(C2CCC2)c(-c2ccc3c(c2)OCCc2ncnc(C4CC4)c2-c2ncc4c(n2)N(CCC4)C3)n1 ; FC(F)(F)c1cn(C2COC2)c(-c2ccc3c(c2)OCCc2ncnc(C4CC4)c2-c2ncc4c(n2)N(CCC4)C3)n1]

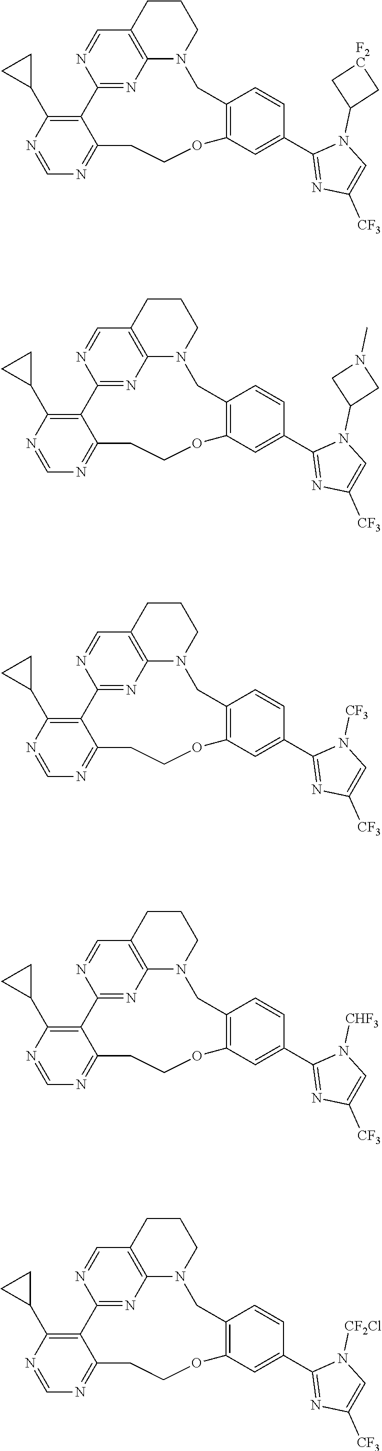

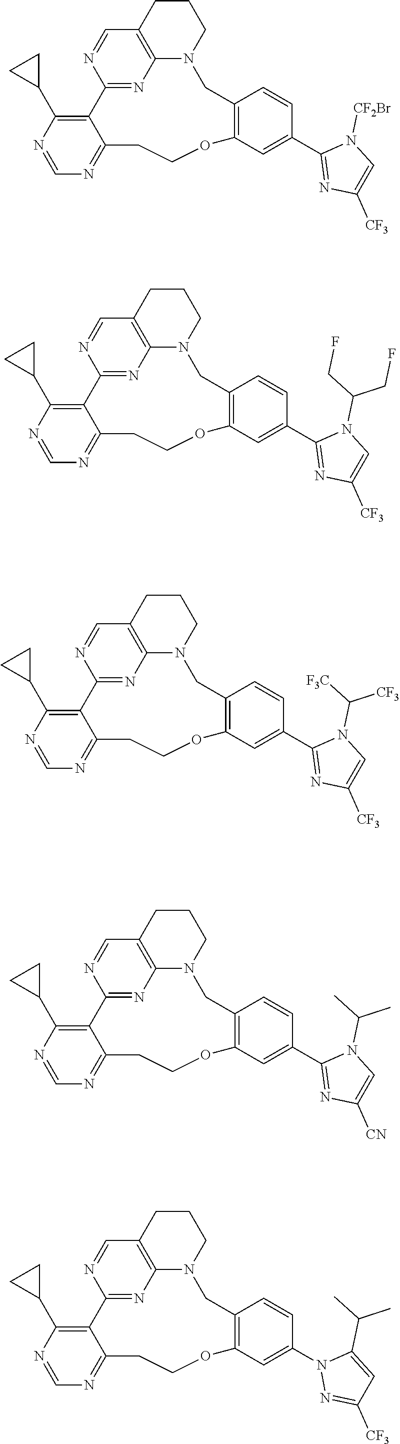

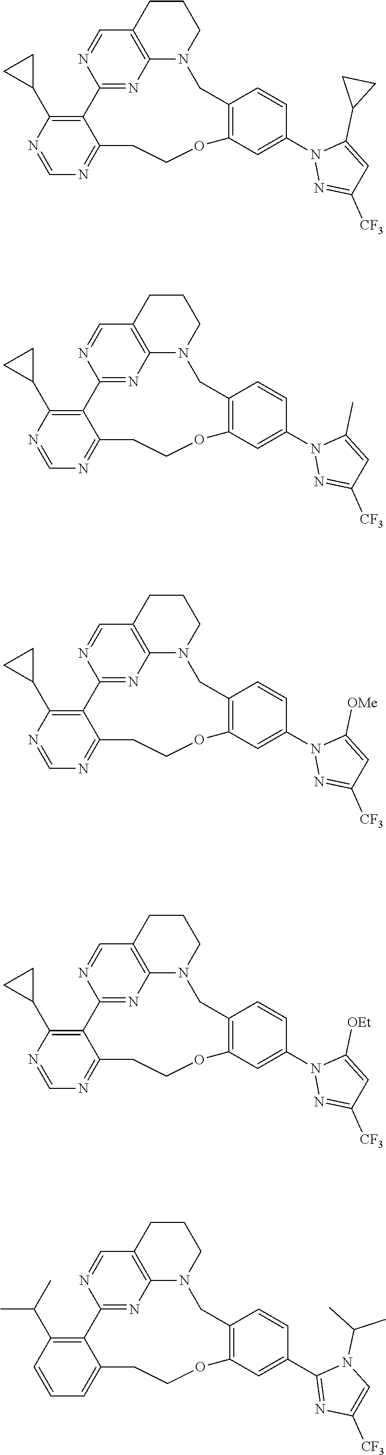 [SMILES: FC(F)(F)c1cc(C2CC2)n(-c2ccc3c(c2)OCCc2ncnc(C4CC4)c2-c2ncc4c(n2)N(CCC4)C3)n1 ; Cc1cc(C(F)(F)F)nn1-c1ccc2c(c1)OCCc1ncnc(C3CC3)c1-c1ncc3c(n1)N(CCC3)C2 ; COc1cc(C(F)(F)F)nn1-c1ccc2c(c1)OCCc1ncnc(C3CC3)c1-c1ncc3c(n1)N(CCC3)C2 ; CCOc1cc(C(F)(F)F)nn1-c1ccc2c(c1)OCCc1ncnc(C3CC3)c1-c1ncc3c(n1)N(CCC3)C2 ; CC(C)c1cccc2c1-c1ncc3c(n1)N(CCC3)Cc1ccc(-c3nc(C(F)(F)F)cn3C(C)C)cc1OCC2]

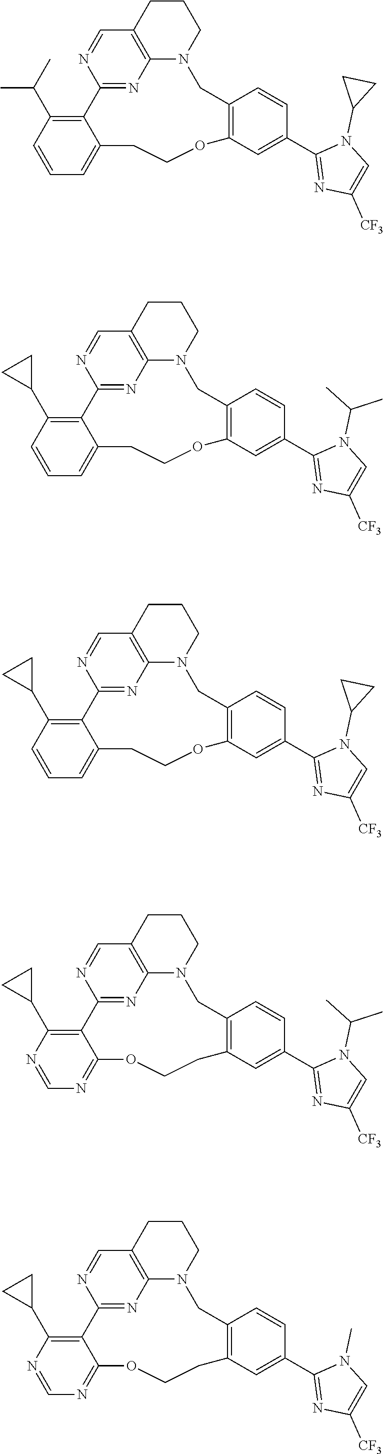 [SMILES: CC(C)c1cccc2c1-c1ncc3c(n1)N(CCC3)Cc1ccc(-c3nc(C(F)(F)F)cn3C3CC3)cc1OCC2 ; CC(C)n1cc(C(F)(F)F)nc1-c1ccc2c(c1)OCCc1cccc(C3CC3)c1-c1ncc3c(n1)N(CCC3)C2 ; FC(F)(F)c1cn(C2CC2)c(-c2ccc3c(c2)OCCc2cccc(C4CC4)c2-c2ncc4c(n2)N(CCC4)C3)n1 ; CC(C)n1cc(C(F)(F)F)nc1-c1ccc2c(c1)CCOc1ncnc(C3CC3)c1-c1ncc3c(n1)N(CCC3)C2 ; Cn1cc(C(F)(F)F)nc1-c1ccc2c(c1)CCOc1ncnc(C3CC3)c1-c1ncc3c(n1)N(CCC3)C2]

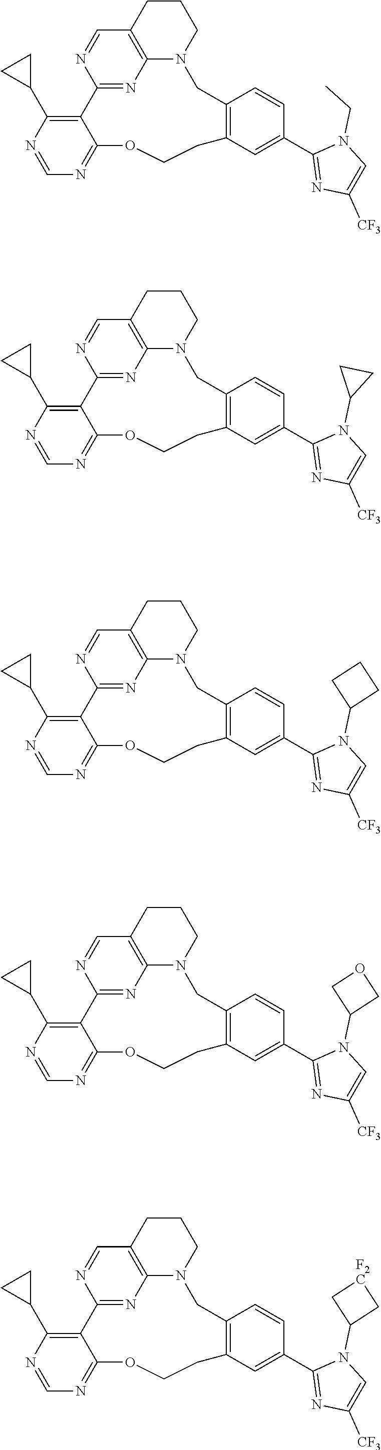 [SMILES: CCn1cc(C(F)(F)F)nc1-c1ccc2c(c1)CCOc1ncnc(C3CC3)c1-c1ncc3c(n1)N(CCC3)C2 ; FC(F)(F)c1cn(C2CC2)c(-c2ccc3c(c2)CCOc2ncnc(C4CC4)c2-c2ncc4c(n2)N(CCC4)C3)n1 ; FC(F)(F)c1cn(C2CCC2)c(-c2ccc3c(c2)CCOc2ncnc(C4CC4)c2-c2ncc4c(n2)N(CCC4)C3)n1 ; FC(F)(F)c1cn(C2COC2)c(-c2ccc3c(c2)CCOc2ncnc(C4CC4)c2-c2ncc4c(n2)N(CCC4)C3)n1 ; FC1(F)CC(n2cc(C(F)(F)F)nc2-c2ccc3c(c2)CCOc2ncnc(C4CC4)c2-c2ncc4c(n2)N(CCC4)C3)C1]

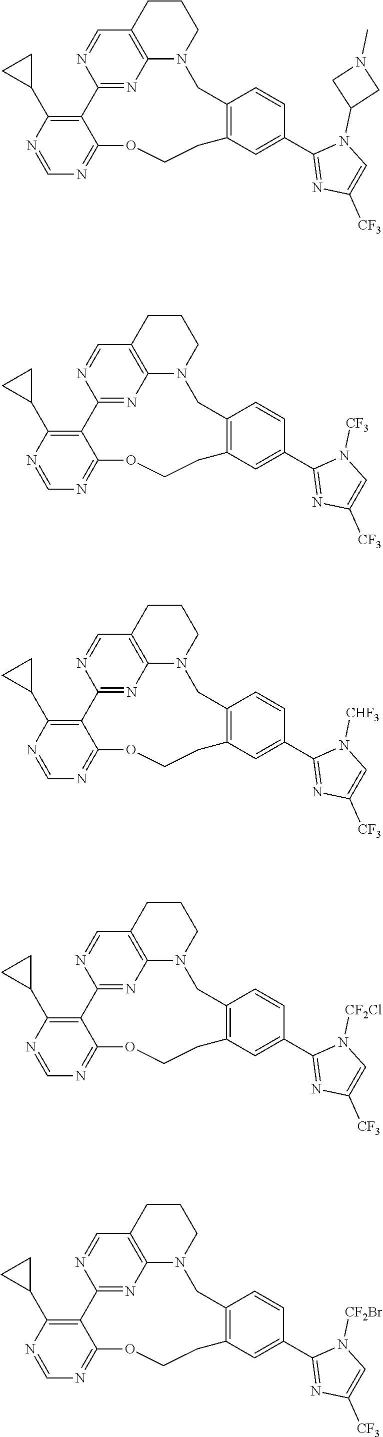

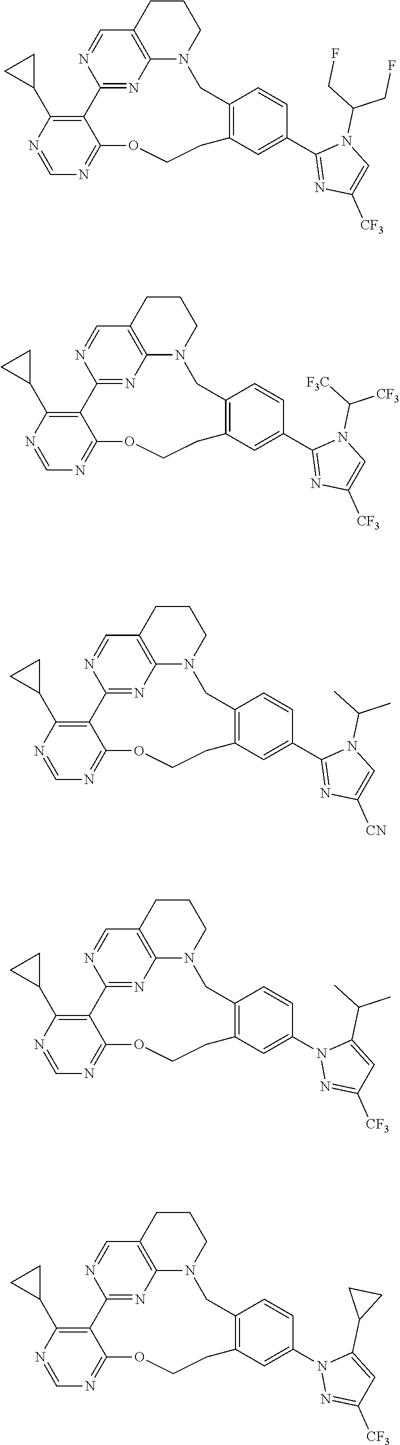 [SMILES: FCC(CF)n1cc(C(F)(F)F)nc1-c1ccc2c(c1)CCOc1ncnc(C3CC3)c1-c1ncc3c(n1)N(CCC3)C2 ; FC(F)(F)c1cn(C(C(F)(F)F)C(F)(F)F)c(-c2ccc3c(c2)CCOc2ncnc(C4CC4)c2-c2ncc4c(n2)N(CCC4)C3)n1 ; CC(C)n1cc(C#N)nc1-c1ccc2c(c1)CCOc1ncnc(C3CC3)c1-c1ncc3c(n1)N(CCC3)C2 ; CC(C)c1cc(C(F)(F)F)nn1-c1ccc2c(c1)CCOc1ncnc(C3CC3)c1-c1ncc3c(n1)N(CCC3)C2 ; FC(F)(F)c1cc(C2CC2)n(-c2ccc3c(c2)CCOc2ncnc(C4CC4)c2-c2ncc4c(n2)N(CCC4)C3)n1]

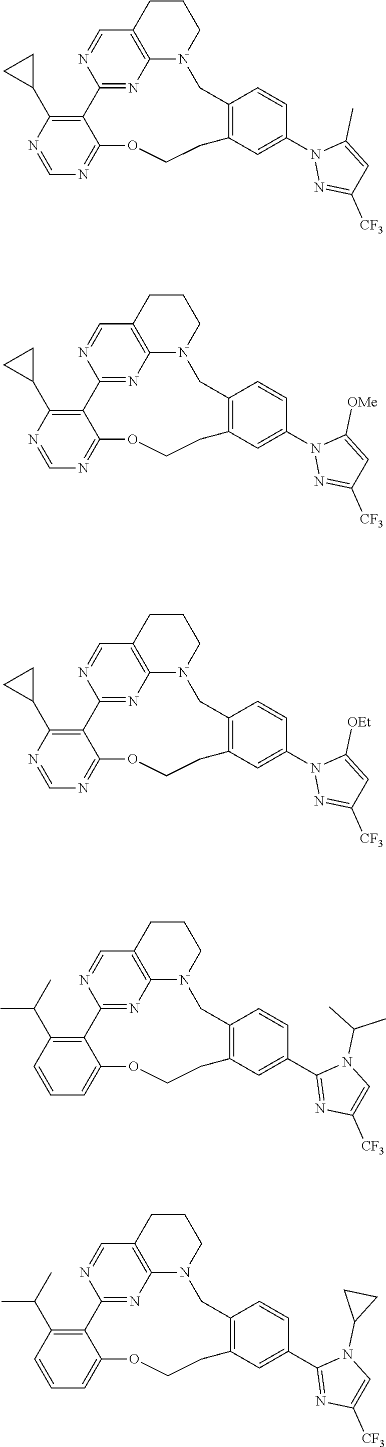

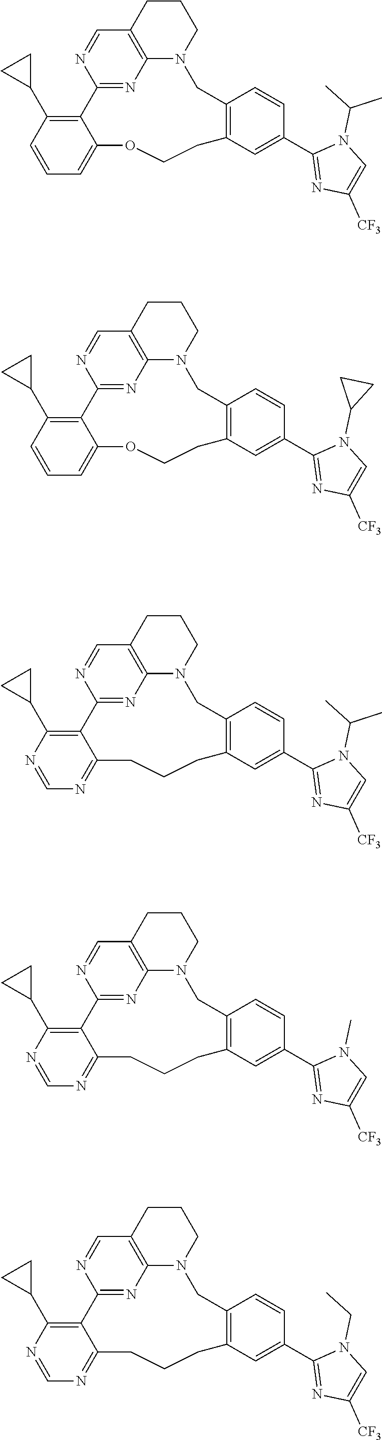 [SMILES: CC(C)n1cc(C(F)(F)F)nc1-c1ccc2c(c1)CCOc1cccc(C3CC3)c1-c1ncc3c(n1)N(CCC3)C2 ; FC(F)(F)c1cn(C2CC2)c(-c2ccc3c(c2)CCOc2cccc(C4CC4)c2-c2ncc4c(n2)N(CCC4)C3)n1 ; CC(C)n1cc(C(F)(F)F)nc1-c1ccc2c(c1)CCCc1ncnc(C3CC3)c1-c1ncc3c(n1)N(CCC3)C2 ; Cn1cc(C(F)(F)F)nc1-c1ccc2c(c1)CCCc1ncnc(C3CC3)c1-c1ncc3c(n1)N(CCC3)C2 ; CCn1cc(C(F)(F)F)nc1-c1ccc2c(c1)CCCc1ncnc(C3CC3)c1-c1ncc3c(n1)N(CCC3)C2]

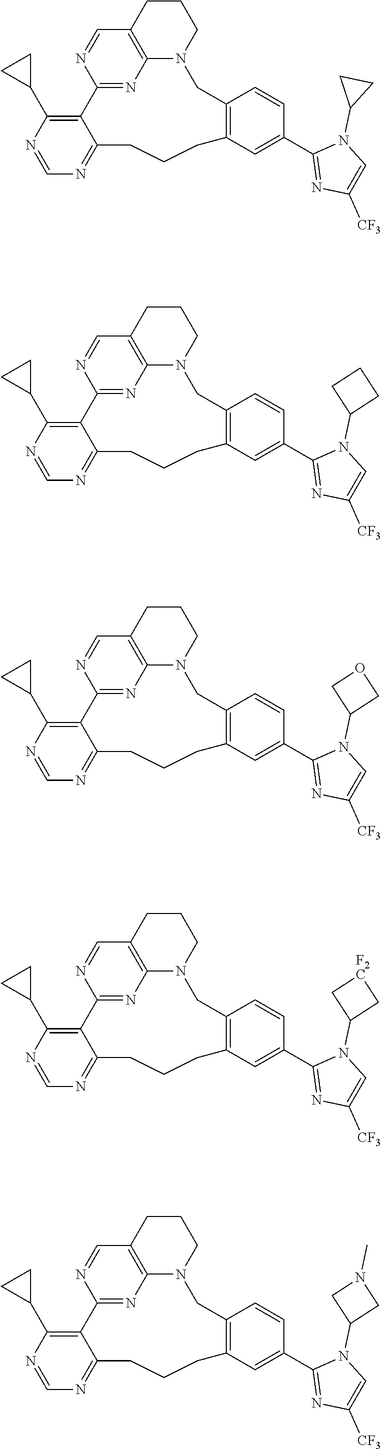 [SMILES: FC(F)(F)c1cn(C2CC2)c(-c2ccc3c(c2)CCCc2ncnc(C4CC4)c2-c2ncc4c(n2)N(CCC4)C3)n1 ; FC(F)(F)c1cn(C2CCC2)c(-c2ccc3c(c2)CCCc2ncnc(C4CC4)c2-c2ncc4c(n2)N(CCC4)C3)n1 ; FC(F)(F)c1cn(C2COC2)c(-c2ccc3c(c2)CCCc2ncnc(C4CC4)c2-c2ncc4c(n2)N(CCC4)C3)n1 ; FC1(F)CC(n2cc(C(F)(F)F)nc2-c2ccc3c(c2)CCCc2ncnc(C4CC4)c2-c2ncc4c(n2)N(CCC4)C3)C1 ; CN1CC(n2cc(C(F)(F)F)nc2-c2ccc3c(c2)CCCc2ncnc(C4CC4)c2-c2ncc4c(n2)N(CCC4)C3)C1]

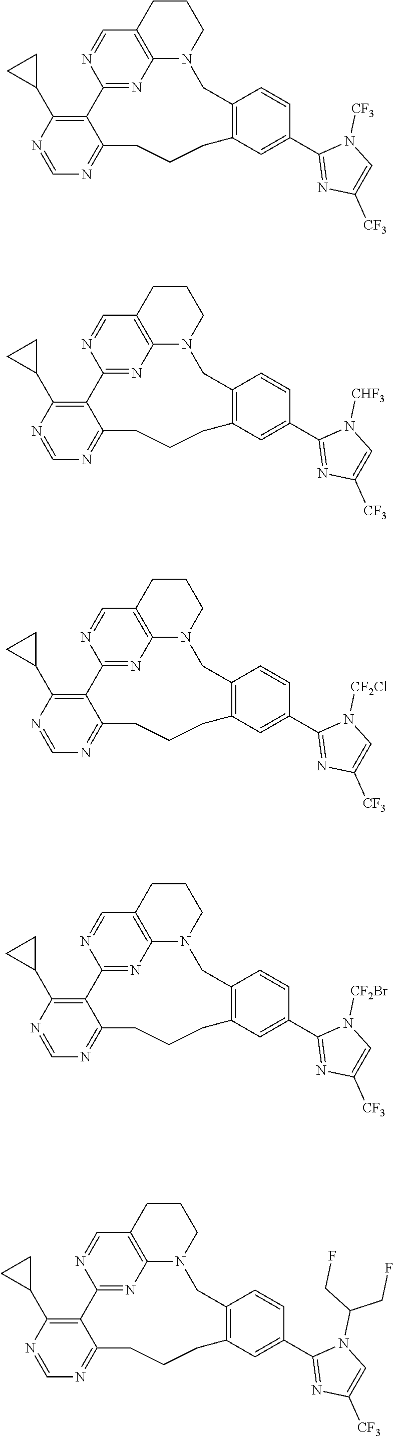

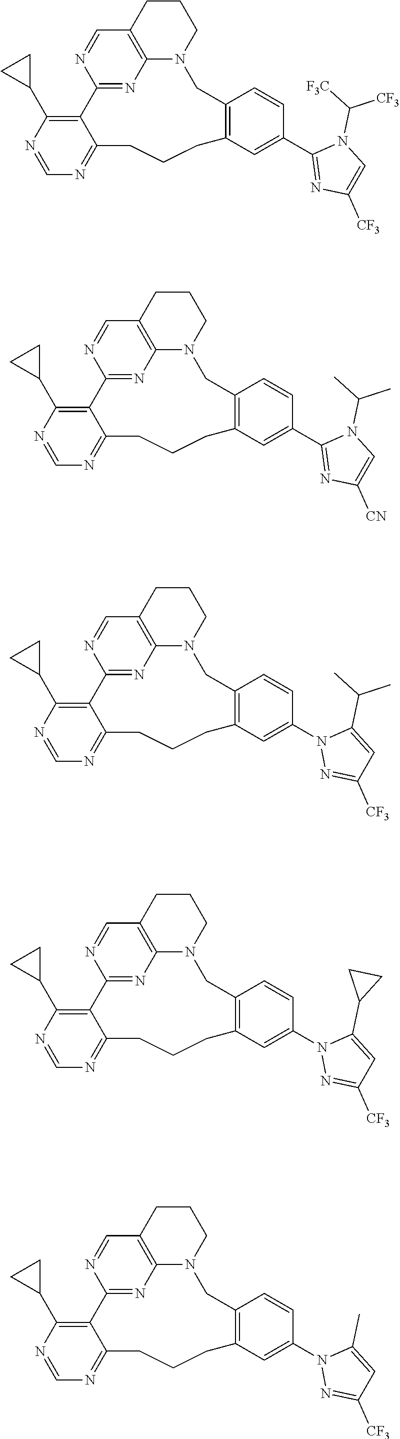 [SMILES: FC(F)(F)c1cn(C(C(F)(F)F)C(F)(F)F)c(-c2ccc3c(c2)CCCc2ncnc(C4CC4)c2-c2ncc4c(n2)N(CCC4)C3)n1 ; CC(C)n1cc(C#N)nc1-c1ccc2c(c1)CCCc1ncnc(C3CC3)c1-c1ncc3c(n1)N(CCC3)C2 ; CC(C)c1cc(C(F)(F)F)nn1-c1ccc2c(c1)CCCc1ncnc(C3CC3)c1-c1ncc3c(n1)N(CCC3)C2 ; FC(F)(F)c1cc(C2CC2)n(-c2ccc3c(c2)CCCc2ncnc(C4CC4)c2-c2ncc4c(n2)N(CCC4)C3)n1 ; Cc1cc(C(F)(F)F)nn1-c1ccc2c(c1)CCCc1ncnc(C3CC3)c1-c1ncc3c(n1)N(CCC3)C2]

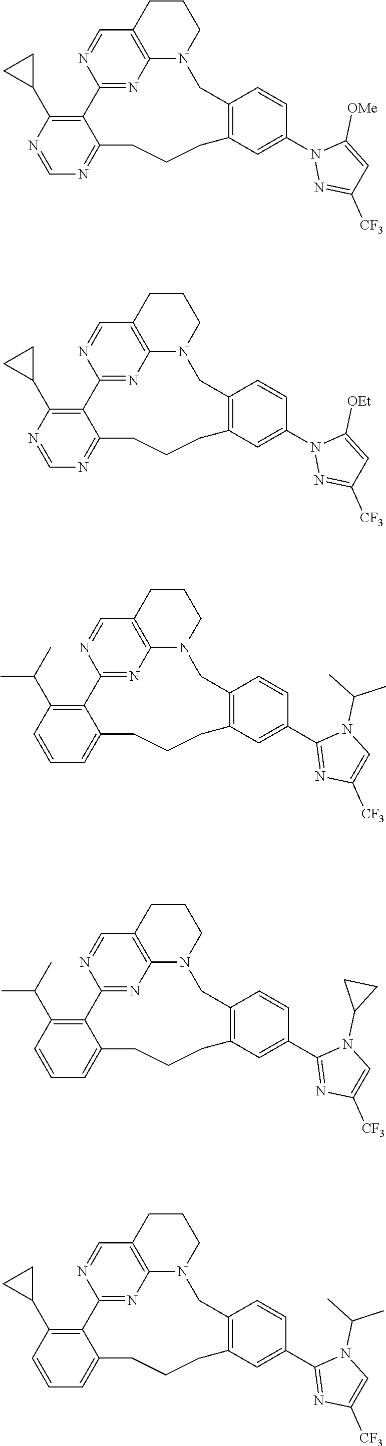

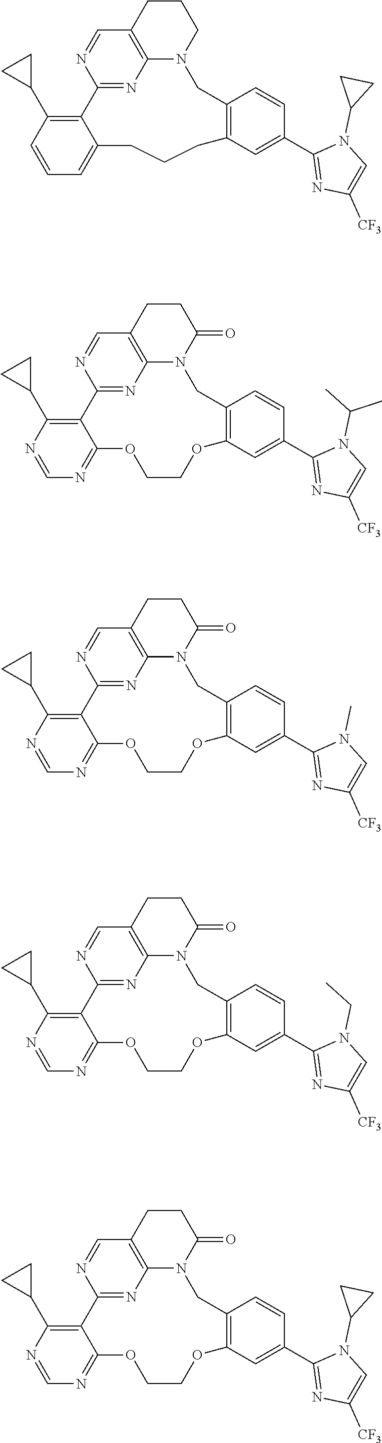 [SMILES: FC(F)(F)c1cn(C2CC2)c(-c2ccc3c(c2)CCCc2cccc(C4CC4)c2-c2ncc4c(n2)N(CCC4)C3)n1 ; CC(C)n1cc(C(F)(F)F)nc1-c1ccc2c(c1)OCCOc1ncnc(C3CC3)c1-c1ncc3c(n1)N(C2)C(=O)CC3 ; Cn1cc(C(F)(F)F)nc1-c1ccc2c(c1)OCCOc1ncnc(C3CC3)c1-c1ncc3c(n1)N(C2)C(=O)CC3 ; CCn1cc(C(F)(F)F)nc1-c1ccc2c(c1)OCCOc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCOc1ncnc(C2CC2)c1-3]

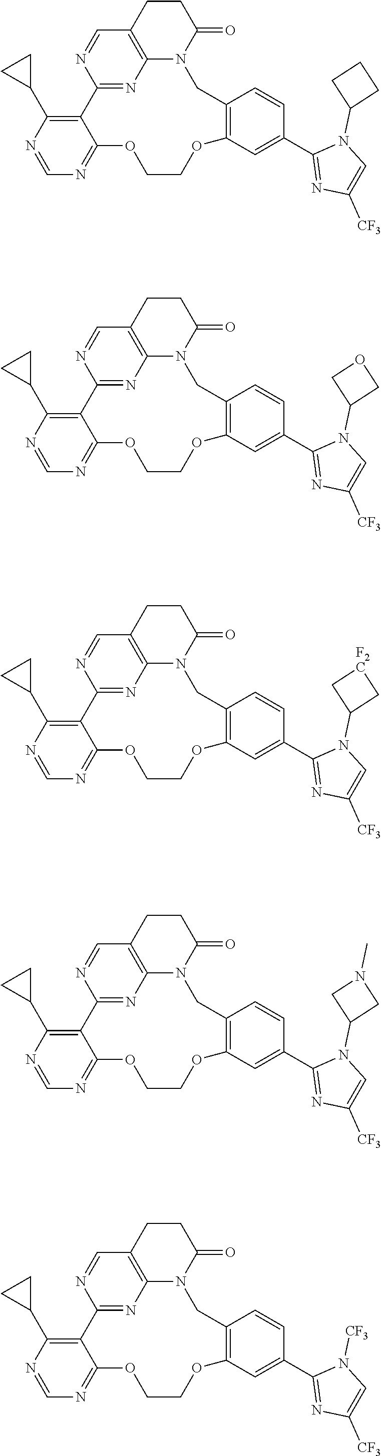 [SMILES: O=C1CCc2cnc3nc2N1Cc1ccc(-c2nc(C(F)(F)F)cn2C2CCC2)cc1OCCOc1ncnc(C2CC2)c1-3 ; O=C1CCc2cnc3nc2N1Cc1ccc(-c2nc(C(F)(F)F)cn2C2COC2)cc1OCCOc1ncnc(C2CC2)c1-3 ; O=C1CCc2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)CC4)C1 ; O=C1CCc2cnc3nc2N1Cc1ccc(-c2nc(C(F)(F)F)cn2C(F)(F)F)cc1OCCOc1ncnc(C2CC2)c1-3]

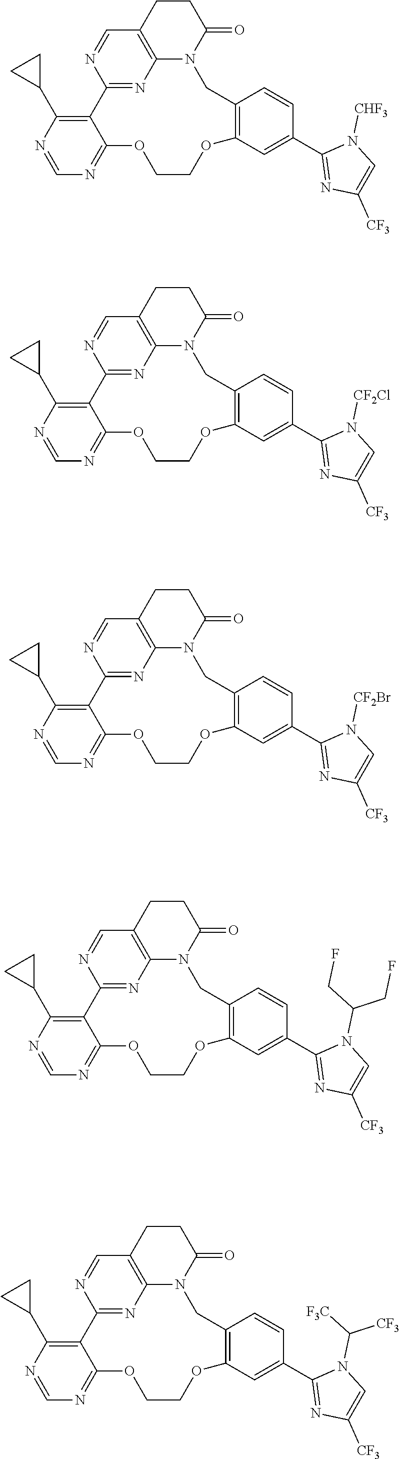

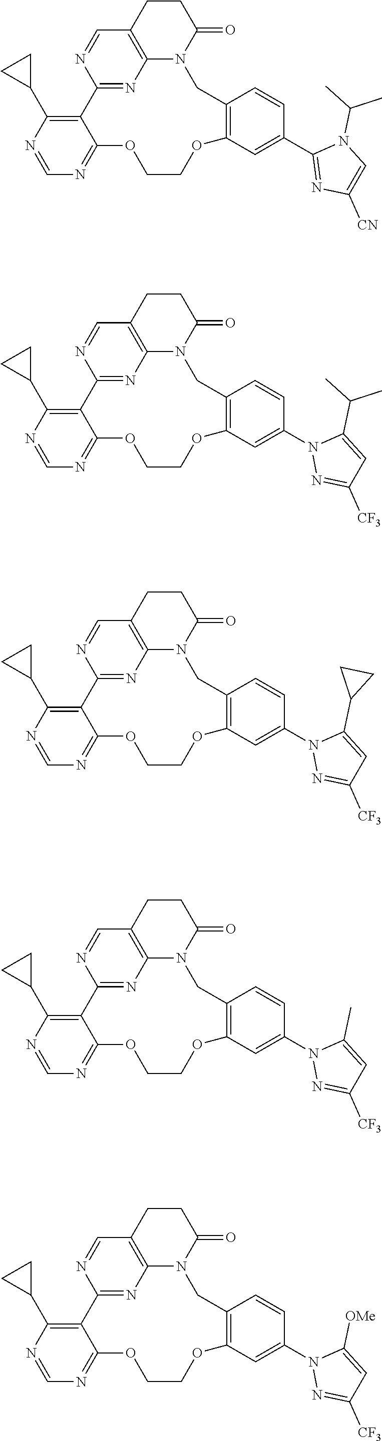 [SMILES: CC(C)n1cc(C#N)nc1-c1ccc2c(c1)OCCOc1ncnc(C3CC3)c1-c1ncc3c(n1)N(C2)C(=O)CC3 ; CC(C)c1cc(C(F)(F)F)nn1-c1ccc2c(c1)OCCOc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)CC3 ; COc1cc(C(F)(F)F)nn1-c1ccc2c(c1)OCCOc1ncnc(C3CC3)c1-c1ncc3c(n1)N(C2)C(=O)CC3]

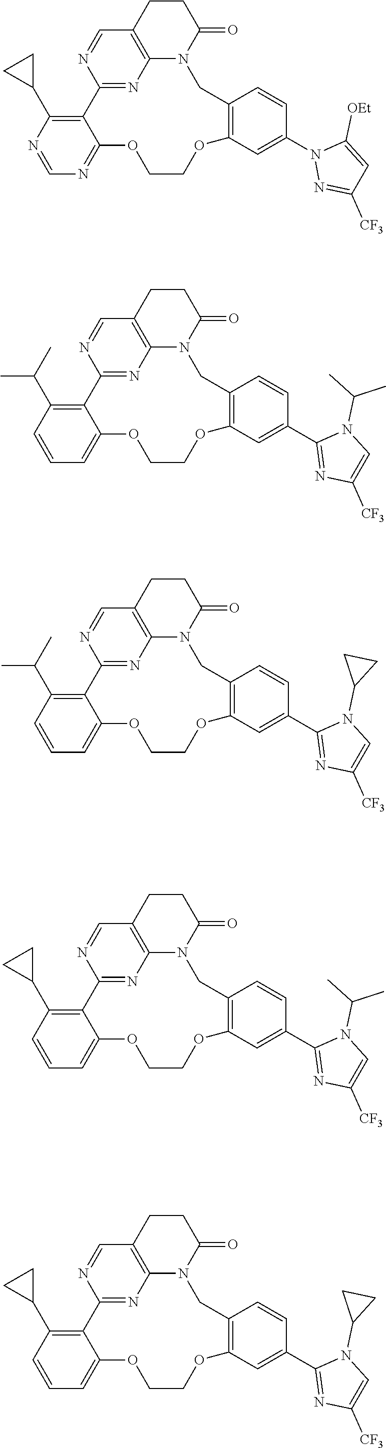

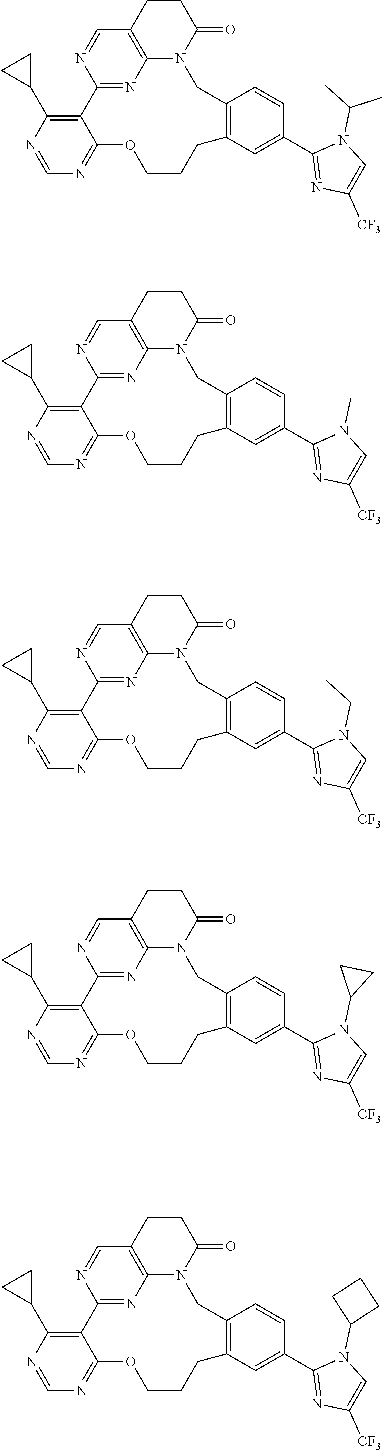 [SMILES: CC(C)n1cc(C(F)(F)F)nc1-c1ccc2c(c1)CCCOc1ncnc(C3CC3)c1-c1ncc3c(n1)N(C2)C(=O)CC3 ; Cn1cc(C(F)(F)F)nc1-c1ccc2c(c1)CCCOc1ncnc(C3CC3)c1-c1ncc3c(n1)N(C2)C(=O)CC3 ; CCn1cc(C(F)(F)F)nc1-c1ccc2c(c1)CCCOc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCOc1ncnc(C2CC2)c1-3 ; O=C1CCc2cnc3nc2N1Cc1ccc(-c2nc(C(F)(F)F)cn2C2CCC2)cc1CCCOc1ncnc(C2CC2)c1-3]

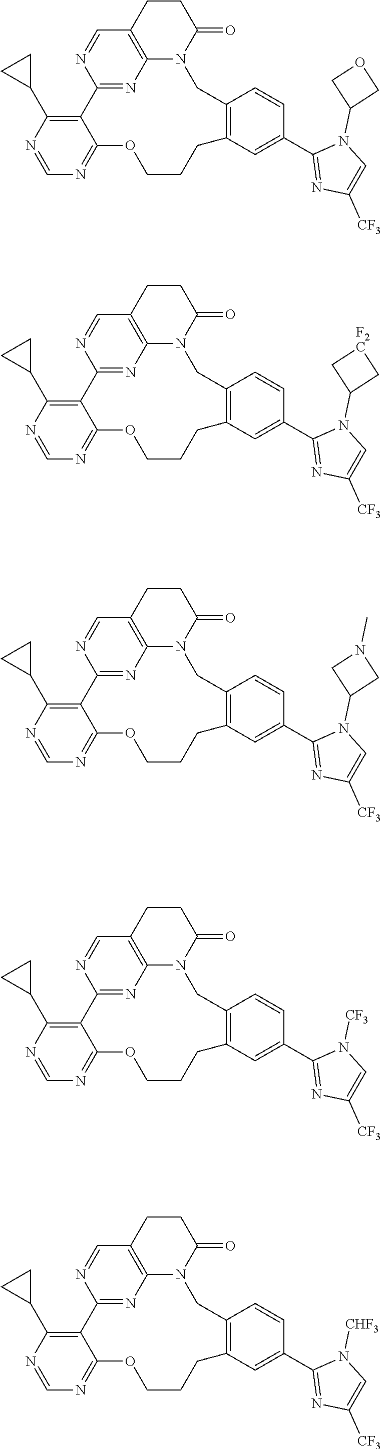

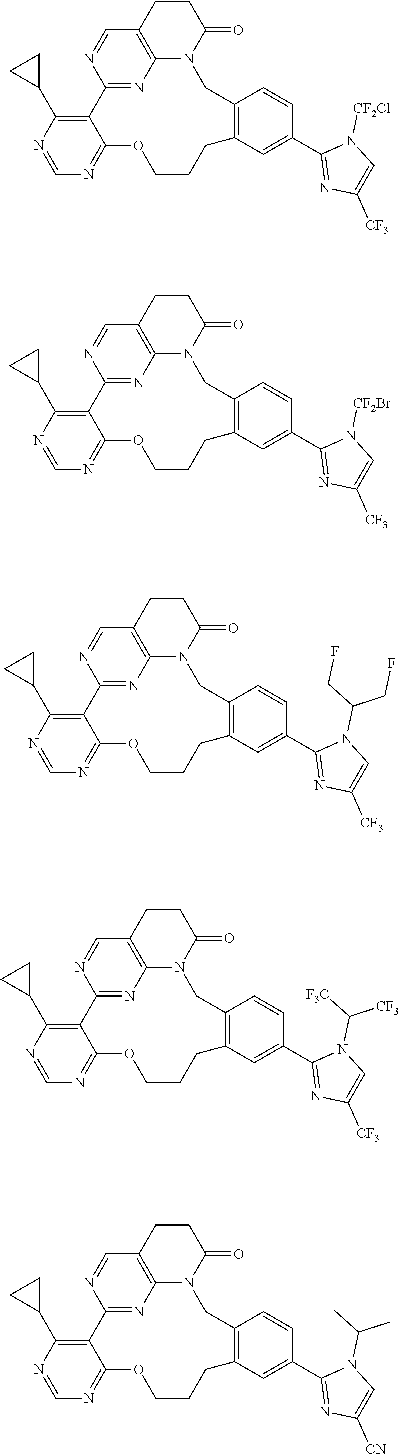

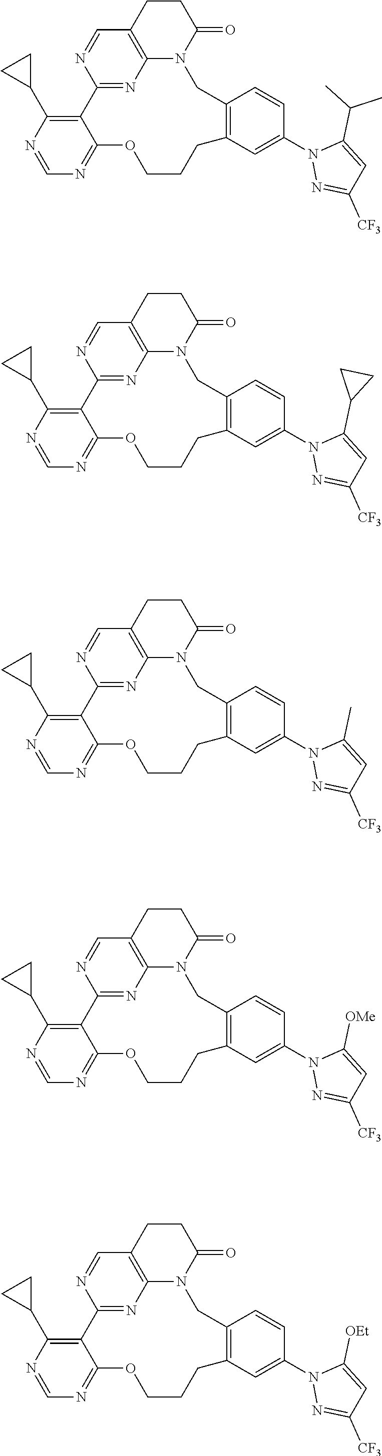 [SMILES: CC(C)c1cc(C(F)(F)F)nn1-c1ccc2c(c1)CCCOc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)CC3 ; COc1cc(C(F)(F)F)nn1-c1ccc2c(c1)CCCOc1ncnc(C3CC3)c1-c1ncc3c(n1)N(C2)C(=O)CC3 ; CCOc1cc(C(F)(F)F)nn1-c1ccc2c(c1)CCCOc1ncnc(C3CC3)c1-c1ncc3c(n1)N(C2)C(=O)CC3]

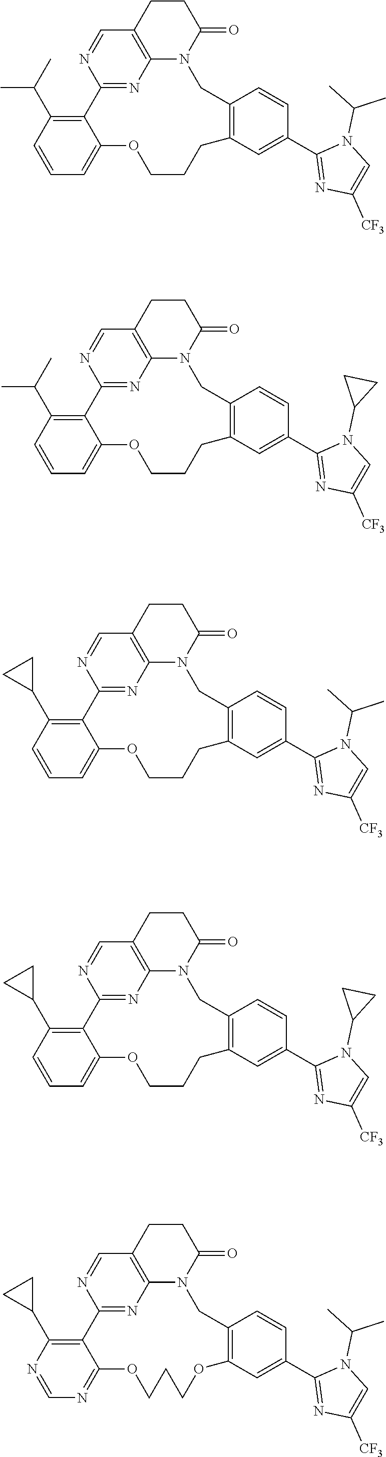

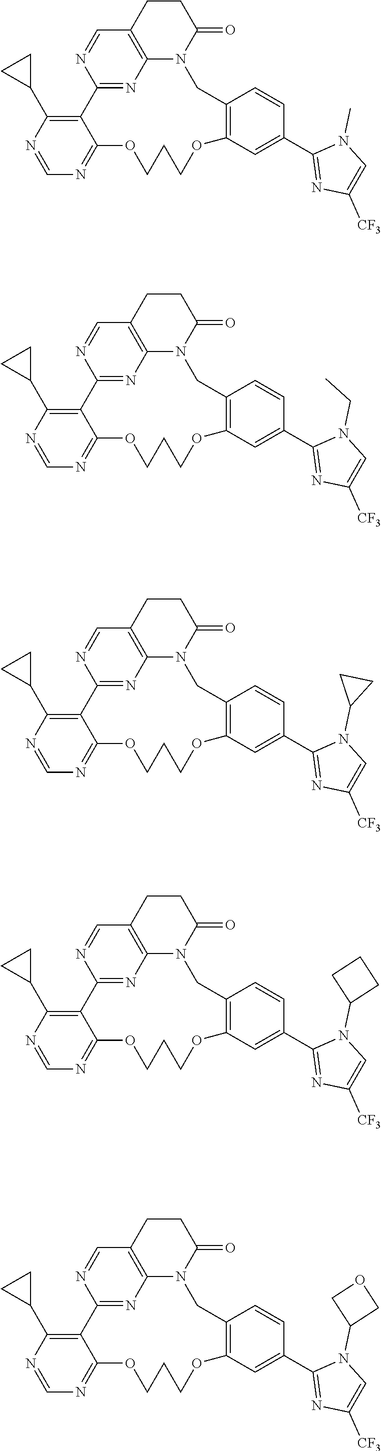 [SMILES: Cn1cc(C(F)(F)F)nc1-c1ccc2c(c1)OCCCOc1ncnc(C3CC3)c1-c1ncc3c(n1)N(C2)C(=O)CC3 ; CCn1cc(C(F)(F)F)nc1-c1ccc2c(c1)OCCCOc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCCOc1ncnc(C2CC2)c1-3 ; O=C1CCc2cnc3nc2N1Cc1ccc(-c2nc(C(F)(F)F)cn2C2CCC2)cc1OCCCOc1ncnc(C2CC2)c1-3 ; O=C1CCc2cnc3nc2N1Cc1ccc(-c2nc(C(F)(F)F)cn2C2COC2)cc1OCCCOc1ncnc(C2CC2)c1-3]

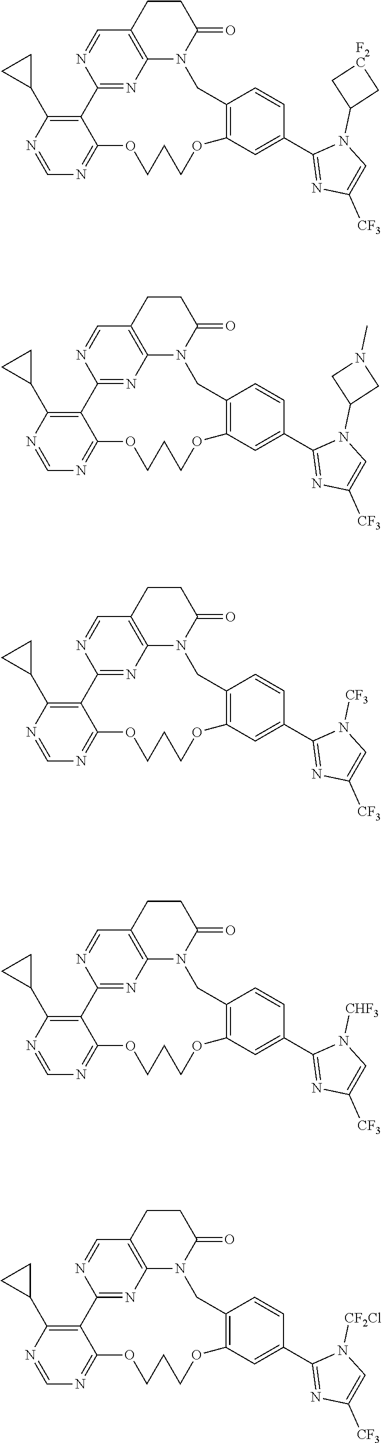

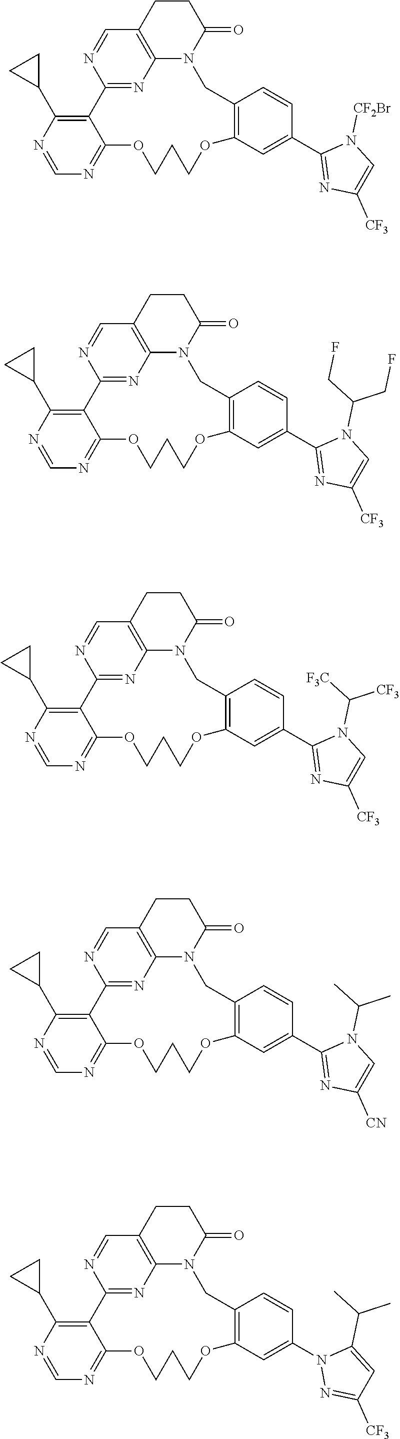

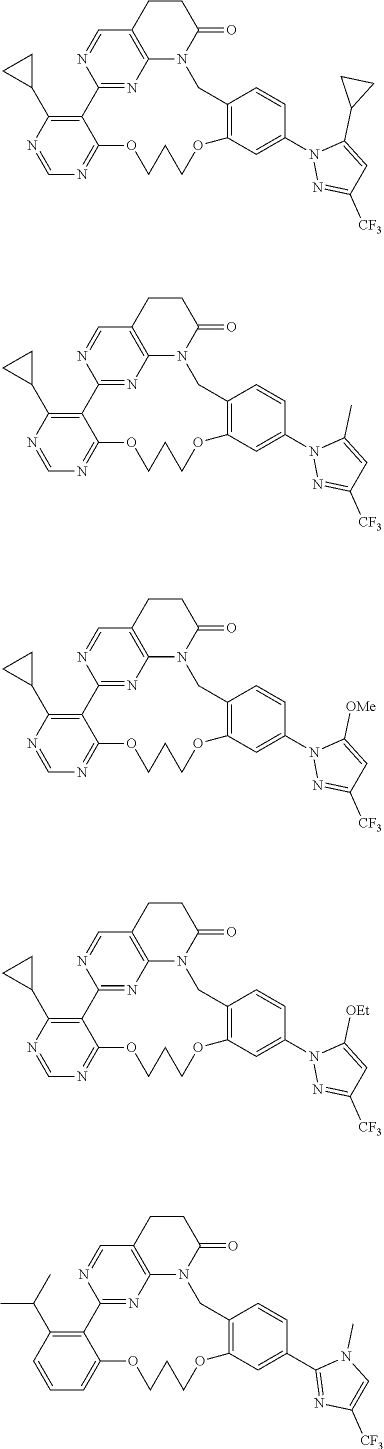 [SMILES: O=C1CCc2cnc3nc2N1Cc1ccc(-n2nc(C(F)(F)F)cc2C2CC2)cc1OCCCOc1ncnc(C2CC2)c1-3 ; Cc1cc(C(F)(F)F)nn1-c1ccc2c(c1)OCCCOc1ncnc(C3CC3)c1-c1ncc3c(n1)N(C2)C(=O)CC3 ; COc1cc(C(F)(F)F)nn1-c1ccc2c(c1)OCCCOc1ncnc(C3CC3)c1-c1ncc3c(n1)N(C2)C(=O)CC3 ; CCOc1cc(C(F)(F)F)nn1-c1ccc2c(c1)OCCCOc1ncnc(C3CC3)c1-c1ncc3c(n1)N(C2)C(=O)CC3 ; CC(C)c1cccc2c1-c1ncc3c(n1)N(Cc1ccc(-c4nc(C(F)(F)F)cn4C)cc1OCCCO2)C(=O)CC3]

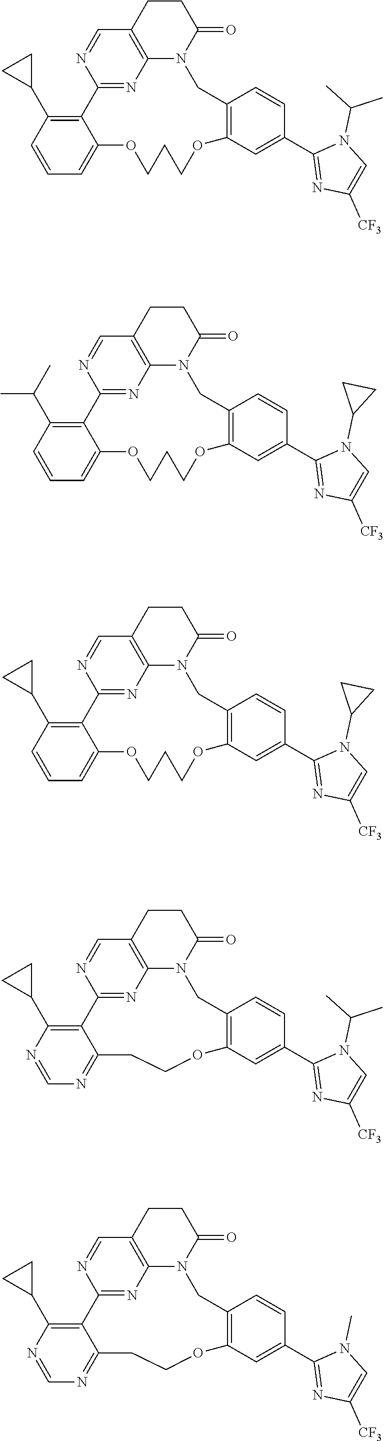

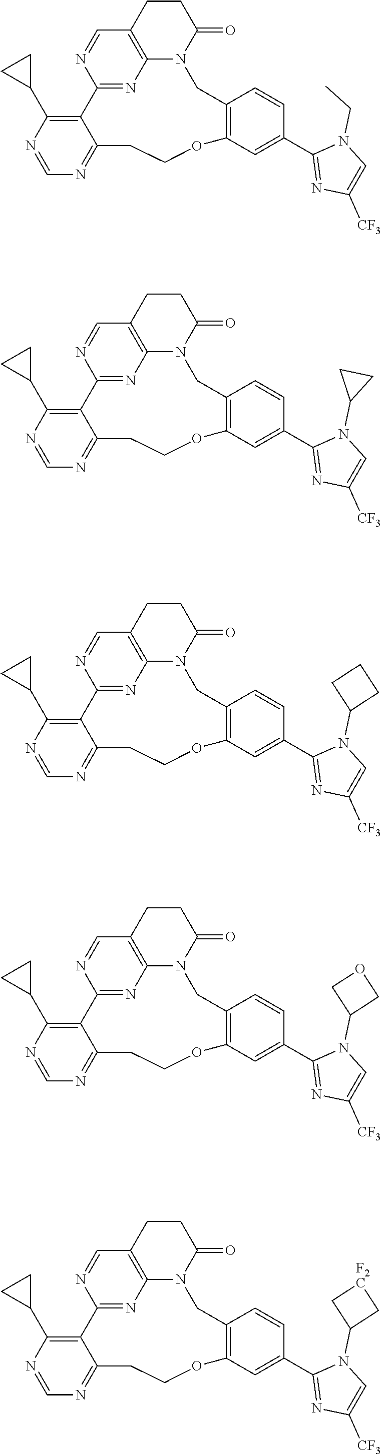 [SMILES: CCn1cc(C(F)(F)F)nc1-c1ccc2c(c1)OCCc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCc1ncnc(C2CC2)c1-3 ; O=C1CCc2cnc3nc2N1Cc1ccc(-c2nc(C(F)(F)F)cn2C2CCC2)cc1OCCc1ncnc(C2CC2)c1-3 ; O=C1CCc2cnc3nc2N1Cc1ccc(-c2nc(C(F)(F)F)cn2C2COC2)cc1OCCc1ncnc(C2CC2)c1-3 ; O=C1CCc2cnc3nc2N1Cc1ccc(-c2nc(C(F)(F)F)cn2C2CC(F)(F)C2)cc1OCCc1ncnc(C2CC2)c1-3]

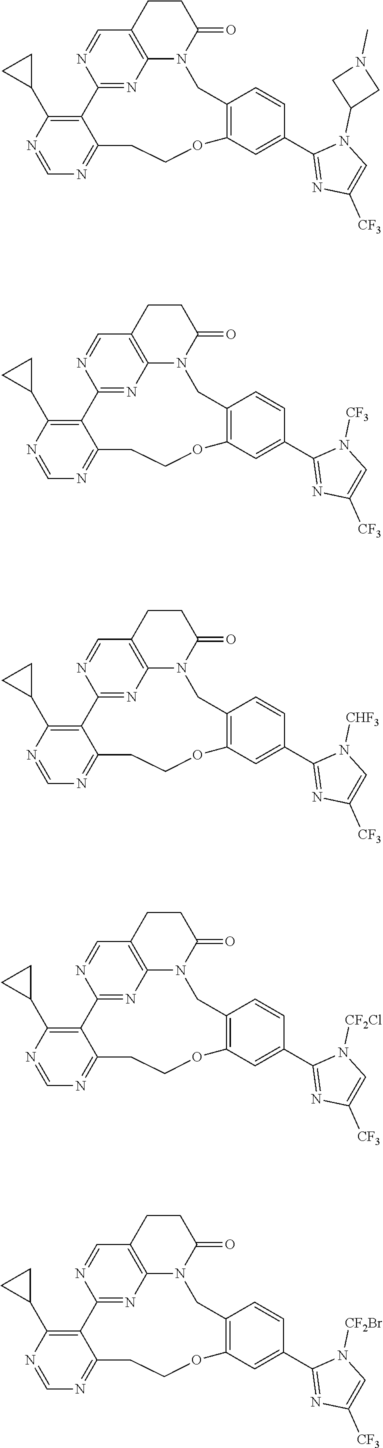

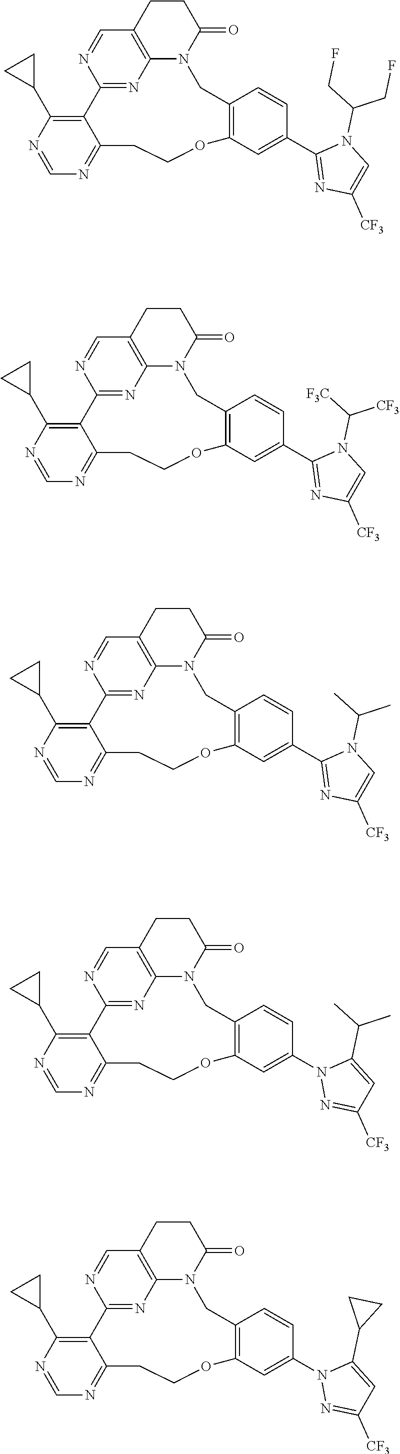 [SMILES: O=C1CCc2cnc3nc2N1Cc1ccc(-c2nc(C(F)(F)F)cn2C(CF)CF)cc1OCCc1ncnc(C2CC2)c1-3 ; O=C1CCc2cnc3nc2N1Cc1ccc(-c2nc(C(F)(F)F)cn2C(C(F)(F)F)C(F)(F)F)cc1OCCc1ncnc(C2CC2)c1-3 ; CC(C)n1cc(C(F)(F)F)nc1-c1ccc2c(c1)OCCc1ncnc(C3CC3)c1-c1ncc3c(n1)N(C2)C(=O)CC3 ; CC(C)c1cc(C(F)(F)F)nn1-c1ccc2c(c1)OCCc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1OCCc1ncnc(C2CC2)c1-3]

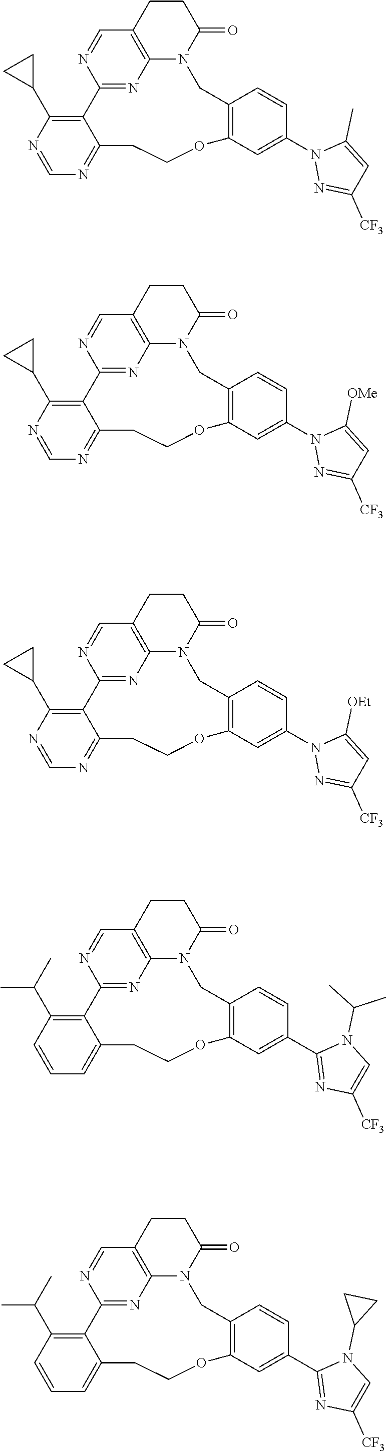

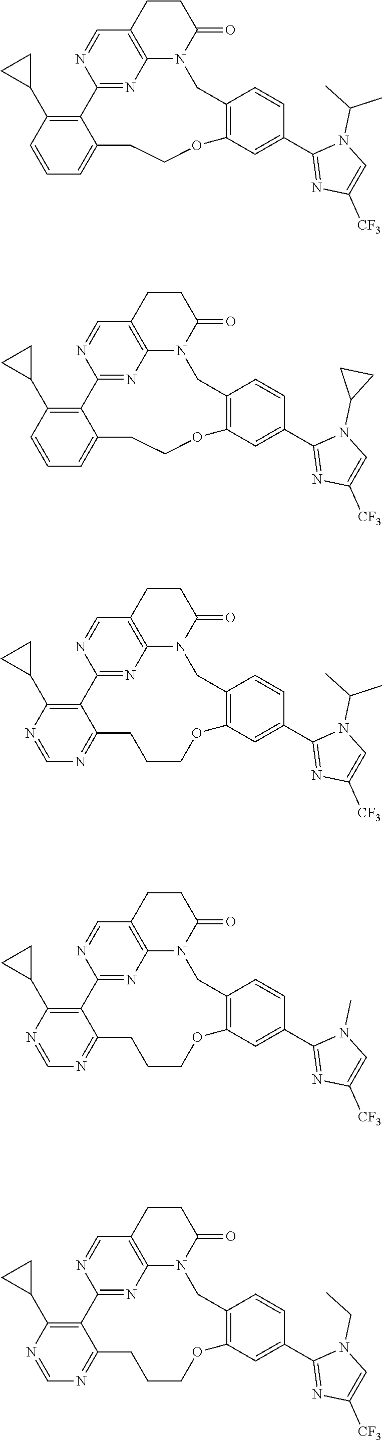 [SMILES: CC(C)n1cc(C(F)(F)F)nc1-c1ccc2c(c1)OCCc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCc1cccc(C2CC2)c1-3 ; CC(C)n1cc(C(F)(F)F)nc1-c1ccc2c(c1)OCCCc1ncnc(C3CC3)c1-c1ncc3c(n1)N(C2)C(=O)CC3 ; Cn1cc(C(F)(F)F)nc1-c1ccc2c(c1)OCCCc1ncnc(C3CC3)c1-c1ncc3c(n1)N(C2)C(=O)CC3 ; CCn1cc(C(F)(F)F)nc1-c1ccc2c(c1)OCCCc1ncnc(C3CC3)c1-c1ncc3c(n1)N(C2)C(=O)CC3]

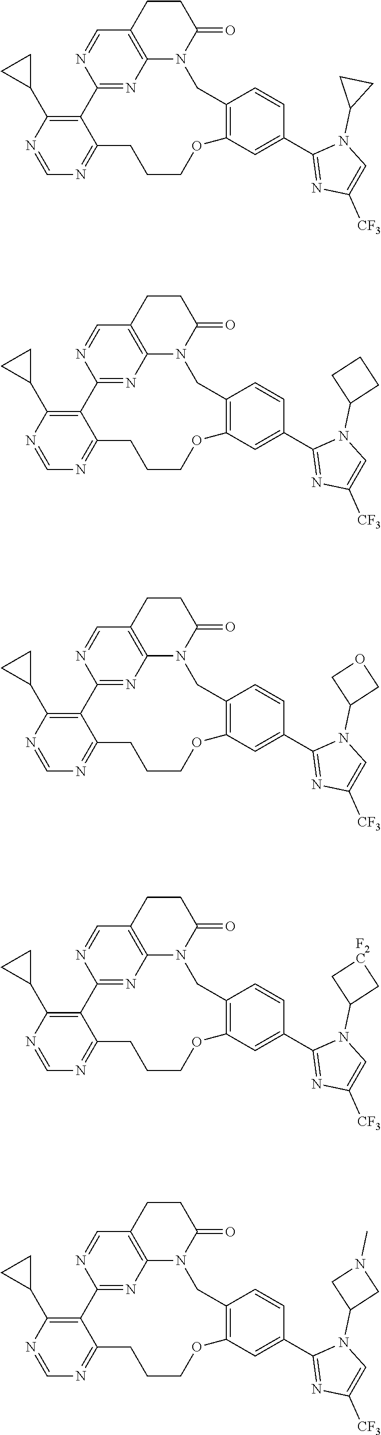 [SMILES: O=C1CCc2cnc3nc2N1Cc1ccc(-c2nc(C(F)(F)F)cn2C2CC2)cc1OCCCc1ncnc(C2CC2)c1-3 ; O=C1CCc2cnc3nc2N1Cc1ccc(-c2nc(C(F)(F)F)cn2C2CCC2)cc1OCCCc1ncnc(C2CC2)c1-3 ; O=C1CCc2cnc3nc2N1Cc1ccc(-c2nc(C(F)(F)F)cn2C2COC2)cc1OCCCc1ncnc(C2CC2)c1-3 ; O=C1CCc2cnc3nc2N1Cc1ccc(-c2nc(C(F)(F)F)cn2C2CC(F)(F)C2)cc1OCCCc1ncnc(C2CC2)c1-3 ; CN1CC(n2cc(C(F)(F)F)nc2-c2ccc3c(c2)OCCCc2ncnc(C4CC4)c2-c2ncc4c(n2)N(C3)C(=O)CC4)C1]

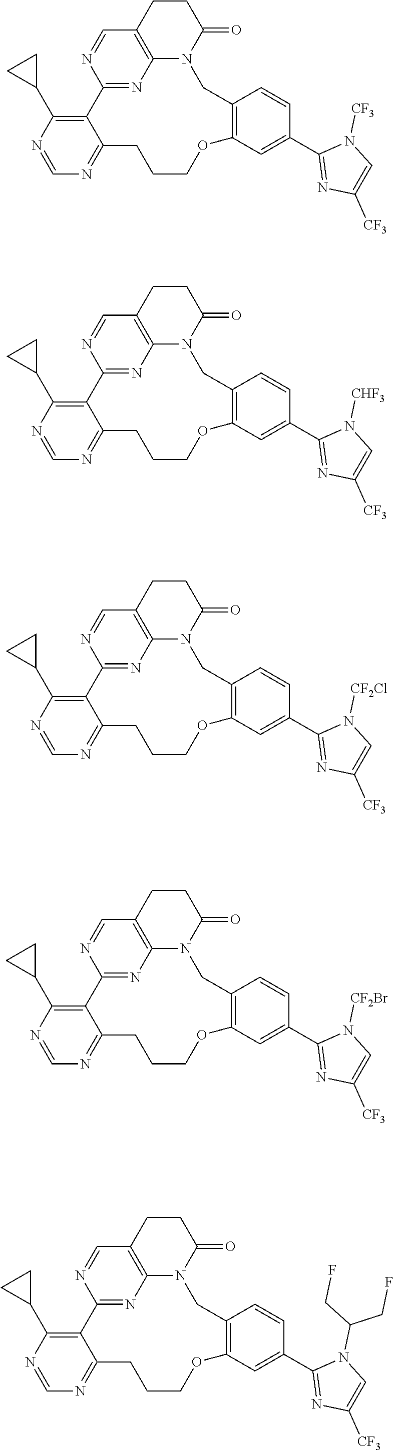

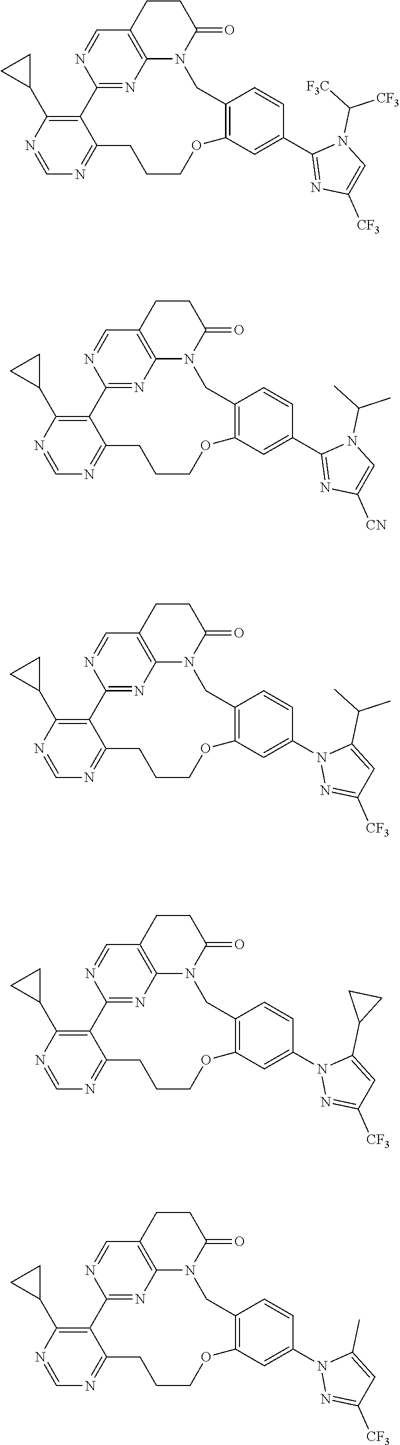 [SMILES: O=C1CCc2cnc3nc2N1Cc1ccc(-c2nc(C(F)(F)F)cn2C(C(F)(F)F)C(F)(F)F)cc1OCCCc1ncnc(C2CC2)c1-3 ; CC(C)n1cc(C#N)nc1-c1ccc2c(c1)OCCCc1ncnc(C3CC3)c1-c1ncc3c(n1)N(C2)C(=O)CC3 ; CC(C)c1cc(C(F)(F)F)nn1-c1ccc2c(c1)OCCCc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1OCCCc1ncnc(C2CC2)c1-3 ; Cc1cc(C(F)(F)F)nn1-c1ccc2c(c1)OCCCc1ncnc(C3CC3)c1-c1ncc3c(n1)N(C2)C(=O)CC3]

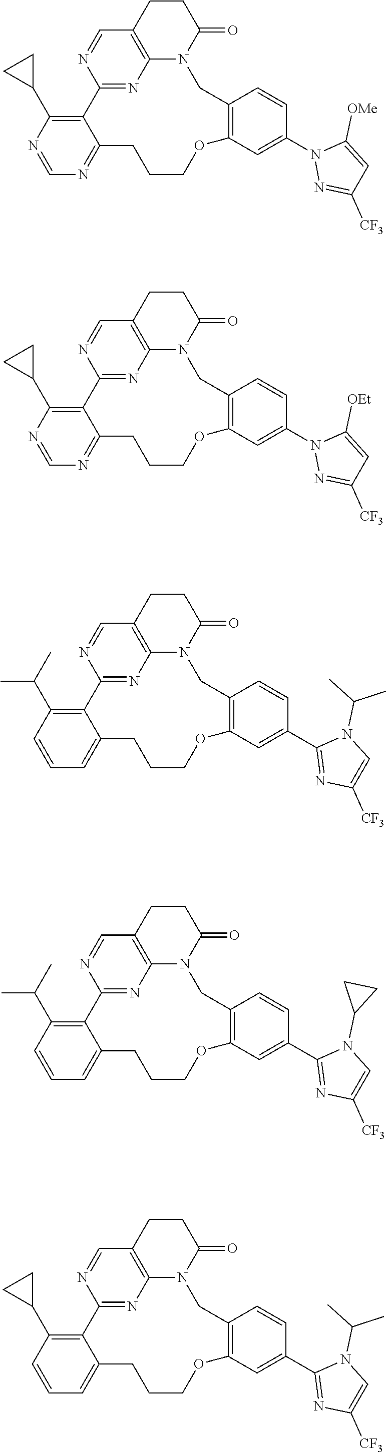

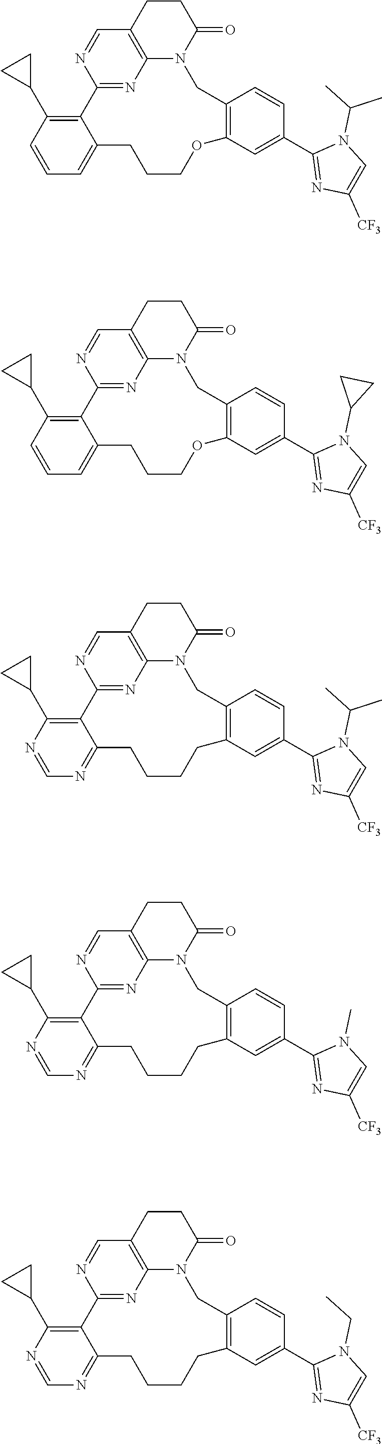 [SMILES: CC(C)n1cc(C(F)(F)F)nc1-c1ccc2c(c1)OCCCc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCCc1cccc(C2CC2)c1-3 ; CC(C)n1cc(C(F)(F)F)nc1-c1ccc2c(c1)CCCCc1ncnc(C3CC3)c1-c1ncc3c(n1)N(C2)C(=O)CC3 ; Cn1cc(C(F)(F)F)nc1-c1ccc2c(c1)CCCCc1ncnc(C3CC3)c1-c1ncc3c(n1)N(C2)C(=O)CC3 ; CCn1cc(C(F)(F)F)nc1-c1ccc2c(c1)CCCCc1ncnc(C3CC3)c1-c1ncc3c(n1)N(C2)C(=O)CC3]

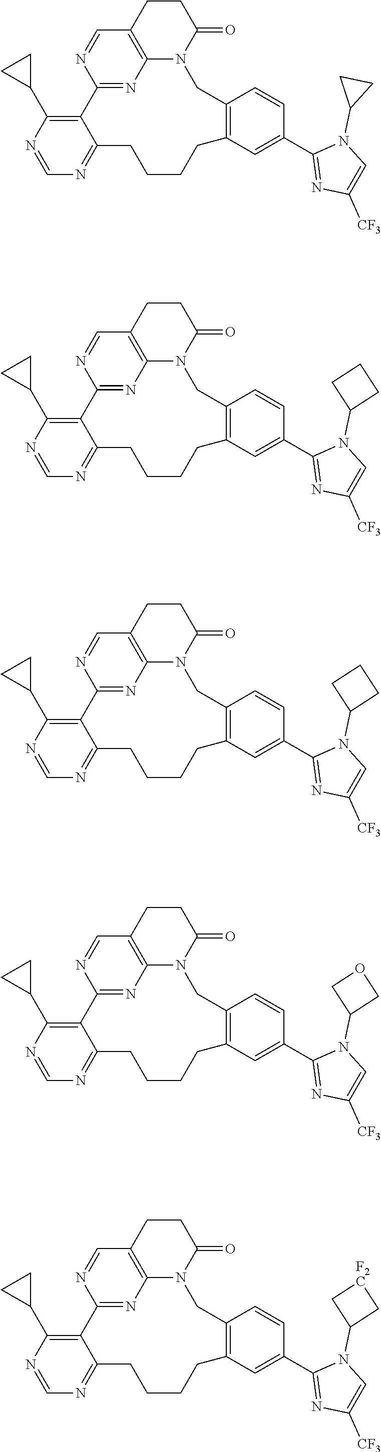 [SMILES: O=C1CCc2cnc3nc2N1Cc1ccc(-c2nc(C(F)(F)F)cn2C2CC2)cc1CCCCc1ncnc(C2CC2)c1-3 ; O=C1CCc2cnc3nc2N1Cc1ccc(-c2nc(C(F)(F)F)cn2C2CCC2)cc1CCCCc1ncnc(C2CC2)c1-3 ; O=C1CCc2cnc3nc2N1Cc1ccc(-c2nc(C(F)(F)F)cn2C2CCC2)cc1CCCCc1ncnc(C2CC2)c1-3 ; O=C1CCc2cnc3nc2N1Cc1ccc(-c2nc(C(F)(F)F)cn2C2COC2)cc1CCCCc1ncnc(C2CC2)c1-3 ; O=C1CCc2cnc3nc2N1Cc1ccc(-c2nc(C(F)(F)F)cn2C2CC(F)(F)C2)cc1CCCCc1ncnc(C2CC2)c1-3]

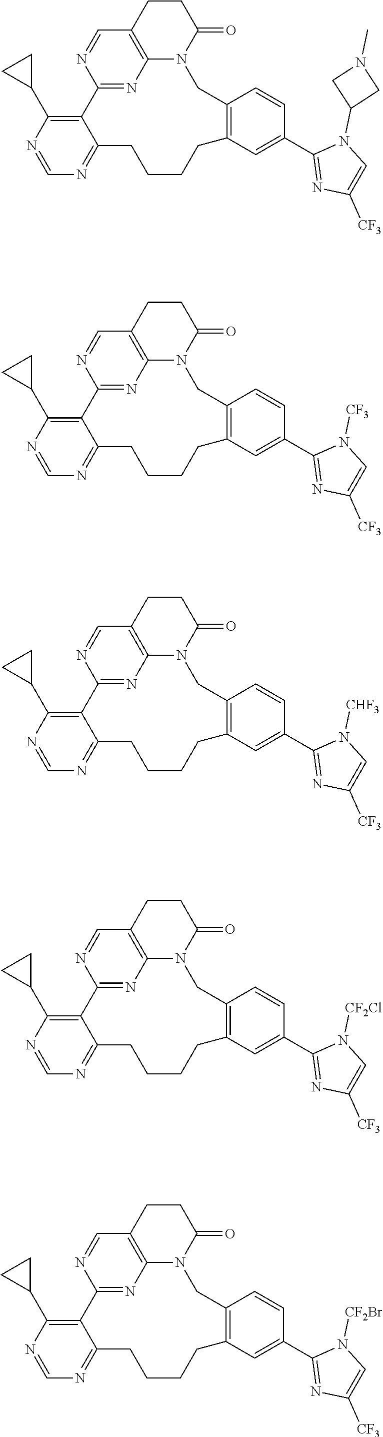

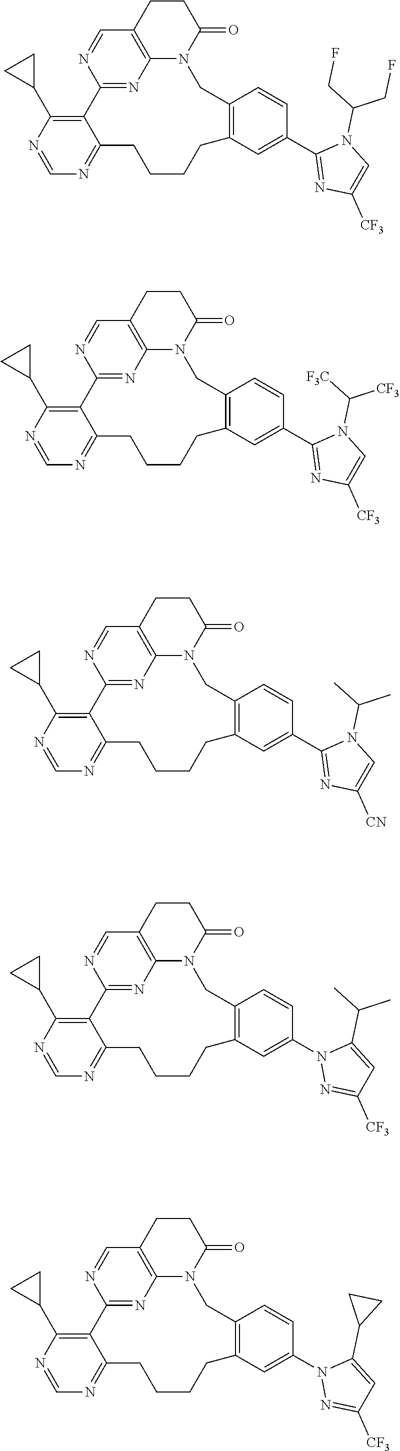 [SMILES: O=C1CCc2cnc3nc2N1Cc1ccc(-c2nc(C(F)(F)F)cn2C(CF)CF)cc1CCCCc1ncnc(C2CC2)c1-3 ; O=C1CCc2cnc3nc2N1Cc1ccc(-c2nc(C(F)(F)F)cn2C(C(F)(F)F)C(F)(F)F)cc1CCCCc1ncnc(C2CC2)c1-3 ; CC(C)n1cc(C#N)nc1-c1ccc2c(c1)CCCCc1ncnc(C3CC3)c1-c1ncc3c(n1)N(C2)C(=O)CC3 ; CC(C)c1cc(C(F)(F)F)nn1-c1ccc2c(c1)CCCCc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1CCCCc1ncnc(C2CC2)c1-3]

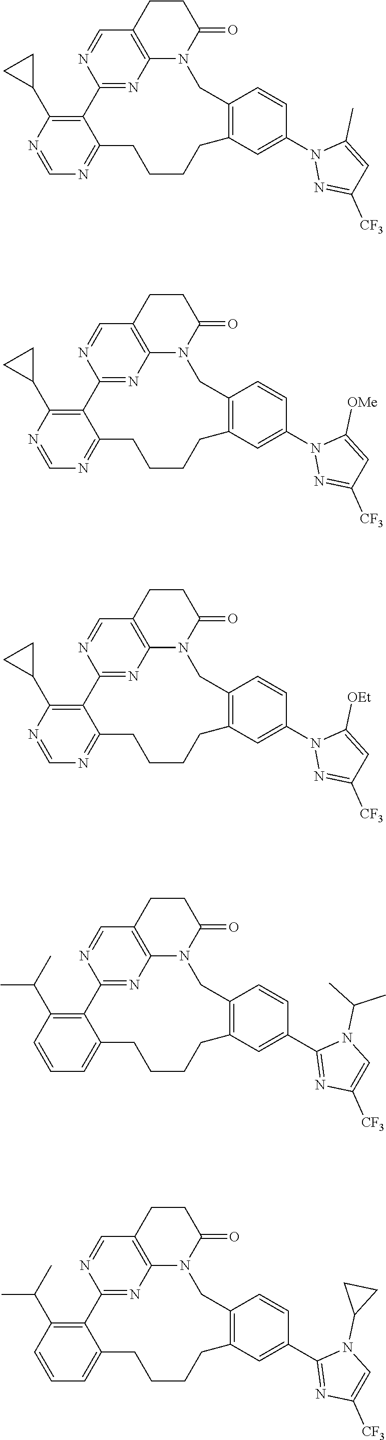

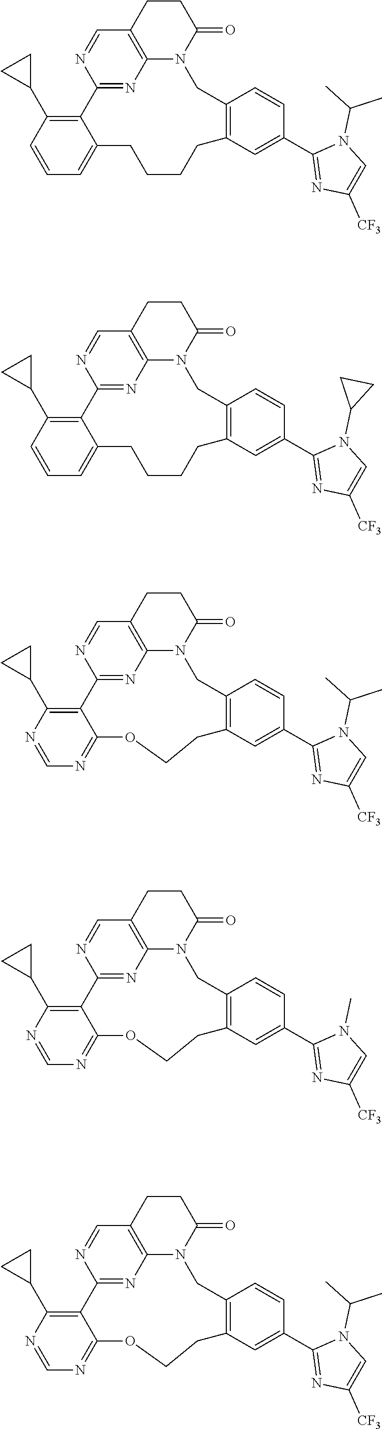 [SMILES: CC(C)n1cc(C(F)(F)F)nc1-c1ccc2c(c1)CCCCc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCCc1cccc(C2CC2)c1-3 ; CC(C)n1cc(C(F)(F)F)nc1-c1ccc2c(c1)CCOc1ncnc(C3CC3)c1-c1ncc3c(n1)N(C2)C(=O)CC3 ; Cn1cc(C(F)(F)F)nc1-c1ccc2c(c1)CCOc1ncnc(C3CC3)c1-c1ncc3c(n1)N(C2)C(=O)CC3 ; CC(C)n1cc(C(F)(F)F)nc1-c1ccc2c(c1)CCOc1ncnc(C3CC3)c1-c1ncc3c(n1)N(C2)C(=O)CC3]

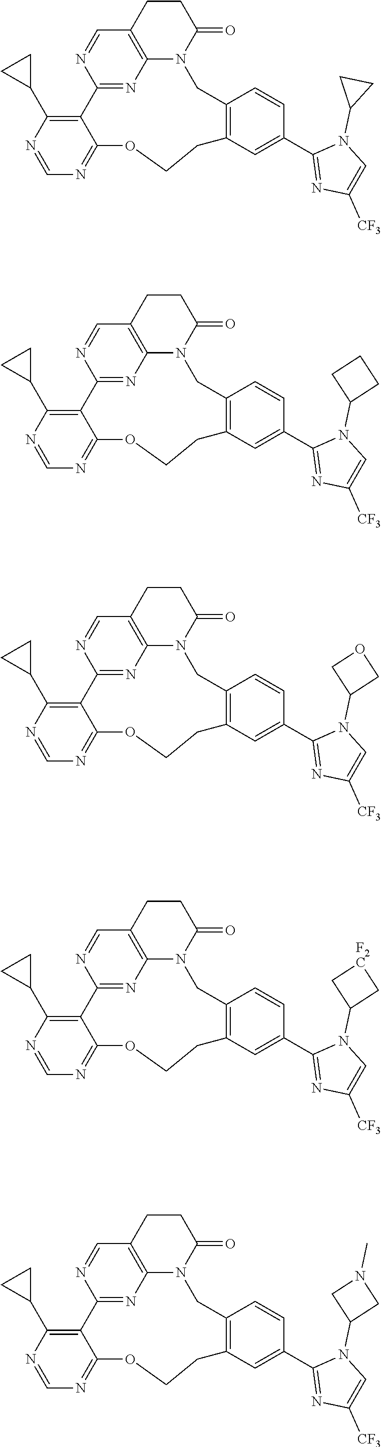 [SMILES: O=C1CCc2cnc3nc2N1Cc1ccc(-c2nc(C(F)(F)F)cn2C2CC2)cc1CCOc1ncnc(C2CC2)c1-3 ; O=C1CCc2cnc3nc2N1Cc1ccc(-c2nc(C(F)(F)F)cn2C2CCC2)cc1CCOc1ncnc(C2CC2)c1-3 ; O=C1CCc2cnc3nc2N1Cc1ccc(-c2nc(C(F)(F)F)cn2C2COC2)cc1CCOc1ncnc(C2CC2)c1-3 ; O=C1CCc2cnc3nc2N1Cc1ccc(-c2nc(C(F)(F)F)cn2C2CC(F)(F)C2)cc1CCOc1ncnc(C2CC2)c1-3 ; CN1CC(n2cc(C(F)(F)F)nc2-c2ccc3c(c2)CCOc2ncnc(C4CC4)c2-c2ncc4c(n2)N(C3)C(=O)CC4)C1]

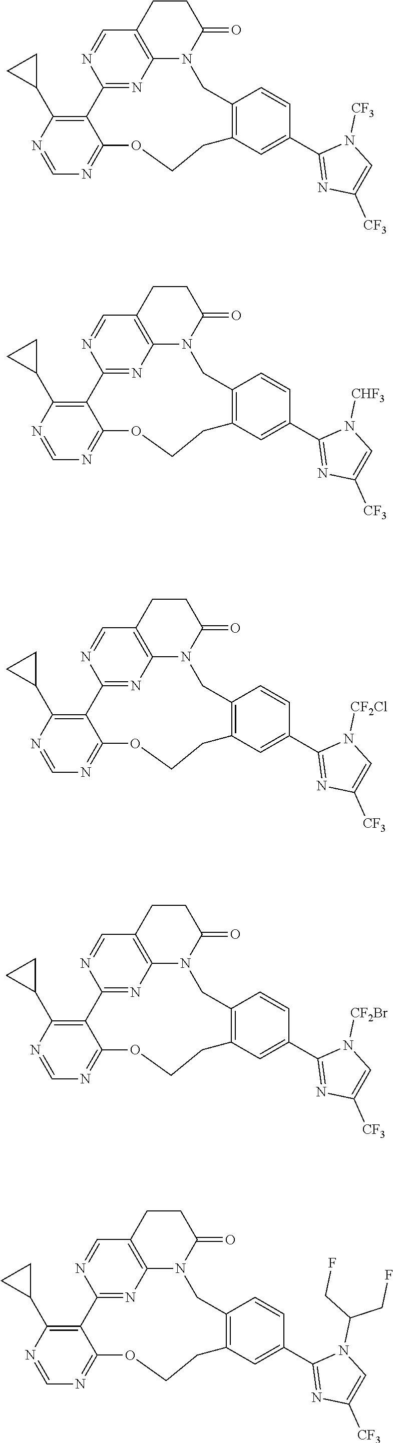

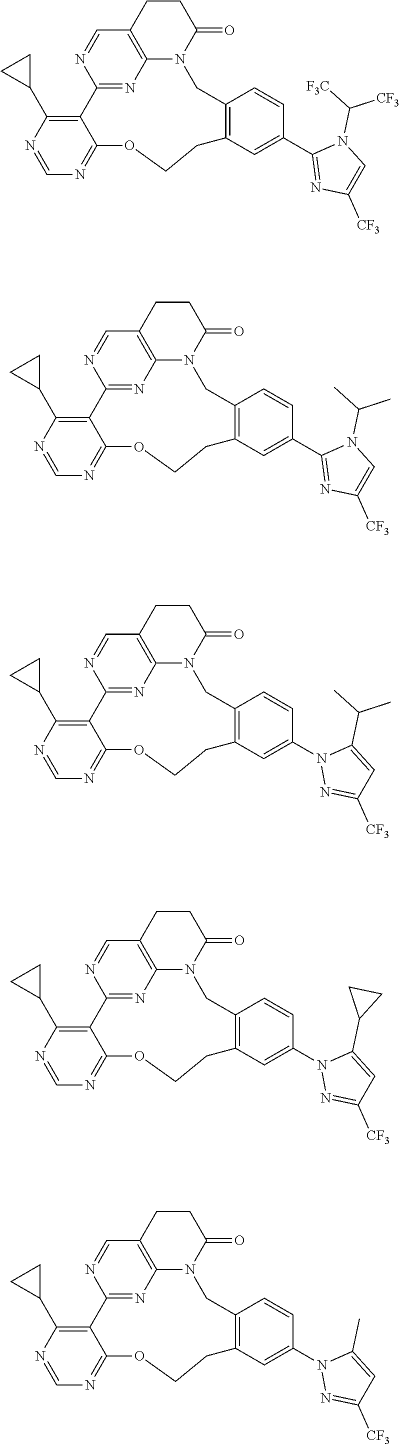 [SMILES: O=C1CCc2cnc3nc2N1Cc1ccc(-c2nc(C(F)(F)F)cn2C(C(F)(F)F)C(F)(F)F)cc1CCOc1ncnc(C2CC2)c1-3 ; CC(C)n1cc(C(F)(F)F)nc1-c1ccc2c(c1)CCOc1ncnc(C3CC3)c1-c1ncc3c(n1)N(C2)C(=O)CC3 ; CC(C)c1cc(C(F)(F)F)nn1-c1ccc2c(c1)CCOc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1CCOc1ncnc(C2CC2)c1-3 ; Cc1cc(C(F)(F)F)nn1-c1ccc2c(c1)CCOc1ncnc(C3CC3)c1-c1ncc3c(n1)N(C2)C(=O)CC3]

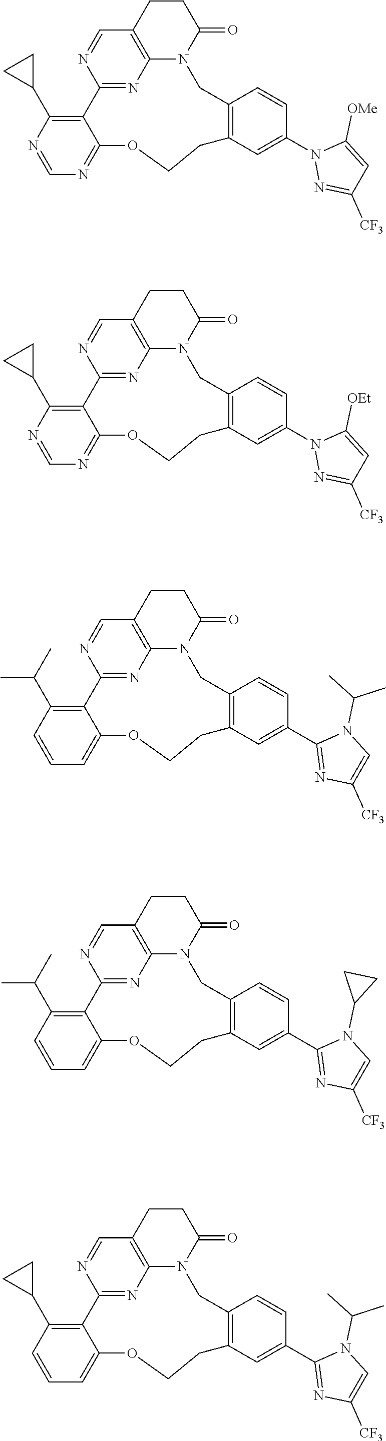

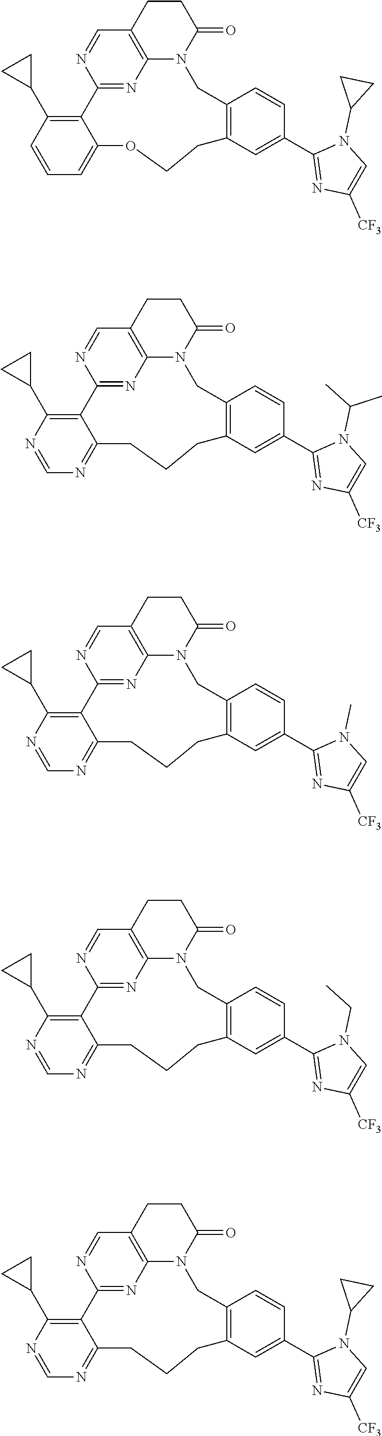 [SMILES: O=C1CCc2cnc3nc2N1Cc1ccc(-c2nc(C(F)(F)F)cn2C2CC2)cc1CCOc1cccc(C2CC2)c1-3 ; CC(C)n1cc(C(F)(F)F)nc1-c1ccc2c(c1)CCCc1ncnc(C3CC3)c1-c1ncc3c(n1)N(C2)C(=O)CC3 ; Cn1cc(C(F)(F)F)nc1-c1ccc2c(c1)CCCc1ncnc(C3CC3)c1-c1ncc3c(n1)N(C2)C(=O)CC3 ; CCn1cc(C(F)(F)F)nc1-c1ccc2c(c1)CCCc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCc1ncnc(C2CC2)c1-3]

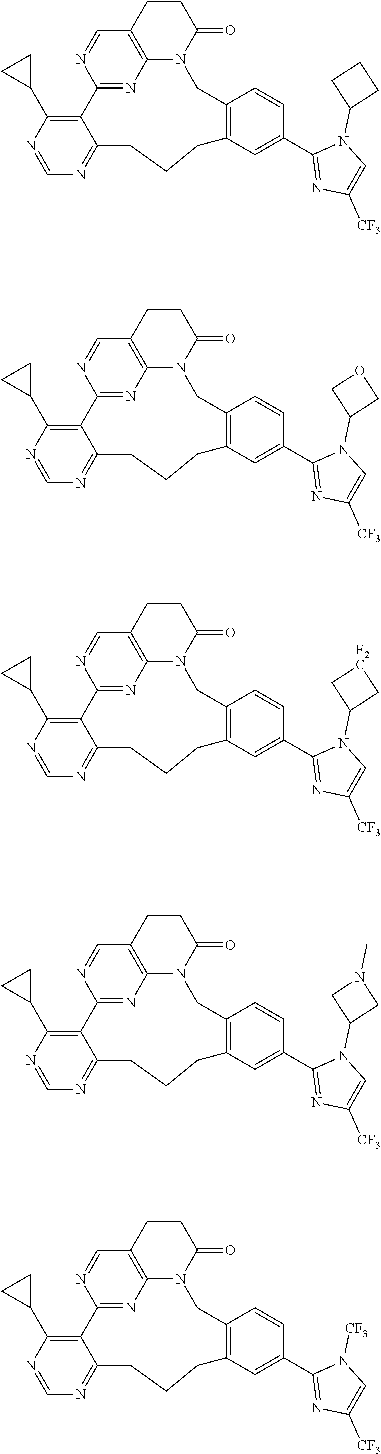 [SMILES: O=C1CCc2cnc3nc2N1Cc1ccc(-c2nc(C(F)(F)F)cn2C2CCC2)cc1CCCc1ncnc(C2CC2)c1-3 ; O=C1CCc2cnc3nc2N1Cc1ccc(-c2nc(C(F)(F)F)cn2C2COC2)cc1CCCc1ncnc(C2CC2)c1-3 ; O=C1CCc2cnc3nc2N1Cc1ccc(-c2nc(C(F)(F)F)cn2C2CC(F)(F)C2)cc1CCCc1ncnc(C2CC2)c1-3 ; CN1CC(n2cc(C(F)(F)F)nc2-c2ccc3c(c2)CCCc2ncnc(C4CC4)c2-c2ncc4c(n2)N(C3)C(=O)CC4)C1 ; O=C1CCc2cnc3nc2N1Cc1ccc(-c2nc(C(F)(F)F)cn2C(F)(F)F)cc1CCCc1ncnc(C2CC2)c1-3]

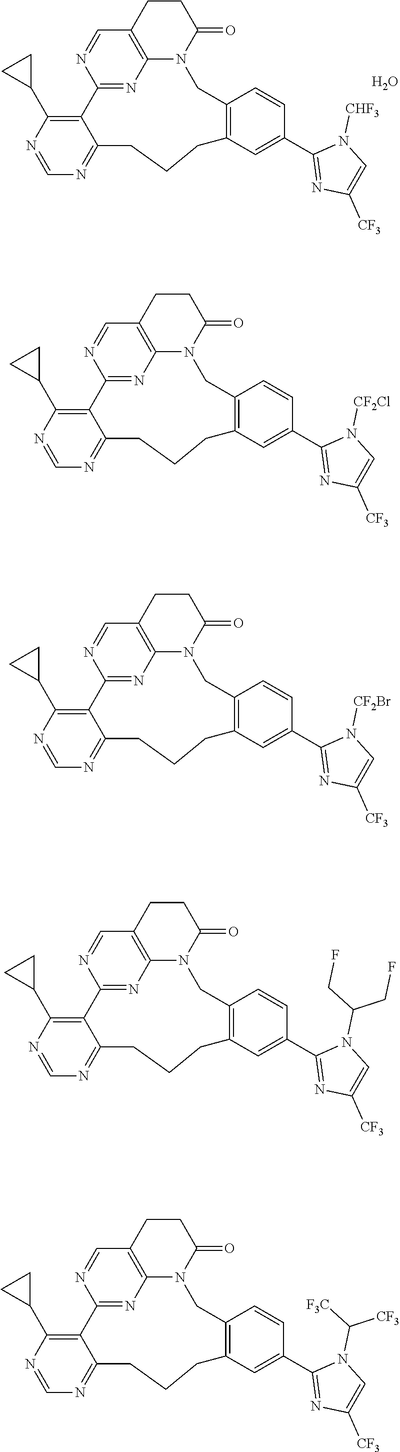

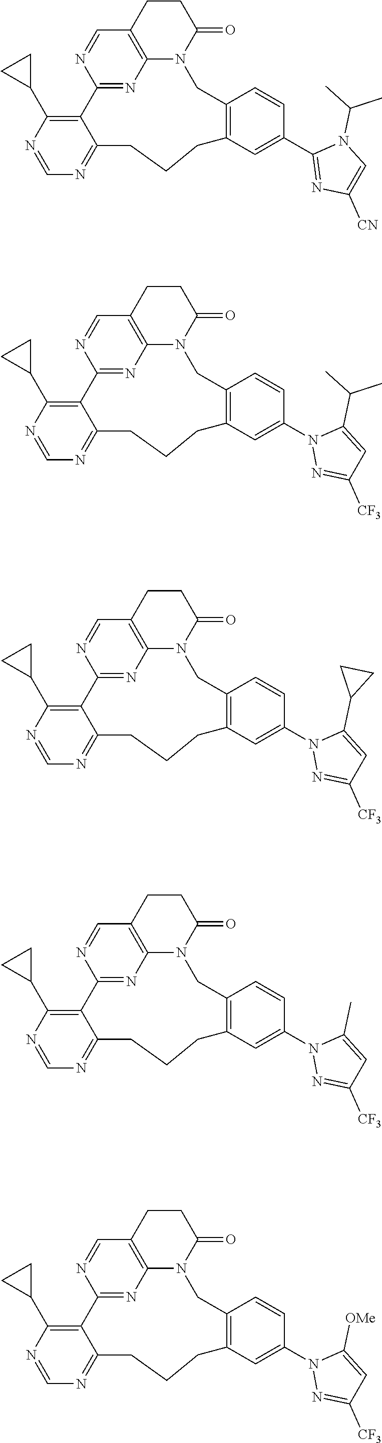 [SMILES: CC(C)n1cc(C#N)nc1-c1ccc2c(c1)CCCc1ncnc(C3CC3)c1-c1ncc3c(n1)N(C2)C(=O)CC3 ; CC(C)c1cc(C(F)(F)F)nn1-c1ccc2c(c1)CCCc1ncnc(C3CC3)c1-c1ncc3c(n1)N(C2)C(=O)CC3 ; O=C1CCc2cnc3nc2N1Cc1ccc(-n2nc(C(F)(F)F)cc2C2CC2)cc1CCCc1ncnc(C2CC2)c1-3 ; Cc1cc(C(F)(F)F)nn1-c1ccc2c(c1)CCCc1ncnc(C3CC3)c1-c1ncc3c(n1)N(C2)C(=O)CC3 ; COc1cc(C(F)(F)F)nn1-c1ccc2c(c1)CCCc1ncnc(C3CC3)c1-c1ncc3c(n1)N(C2)C(=O)CC3]

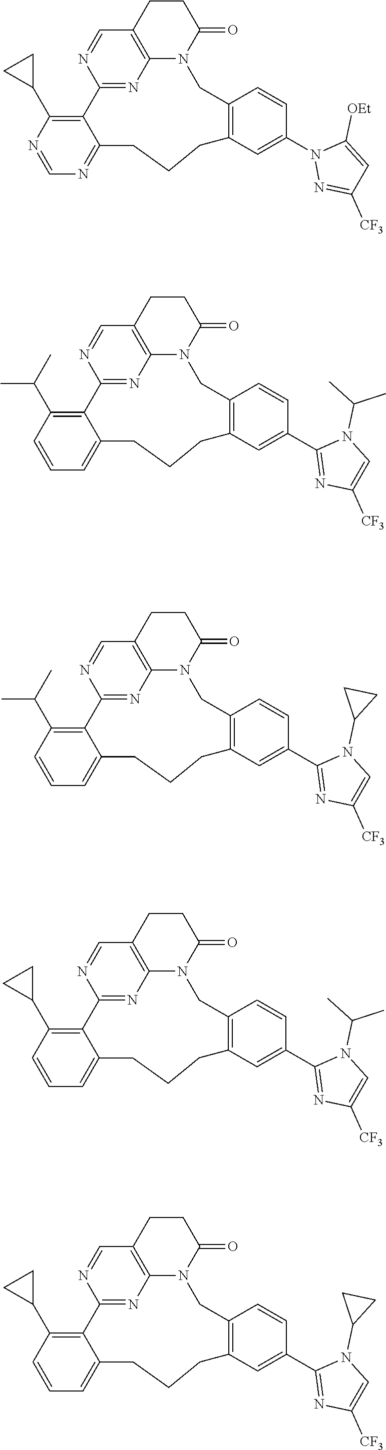

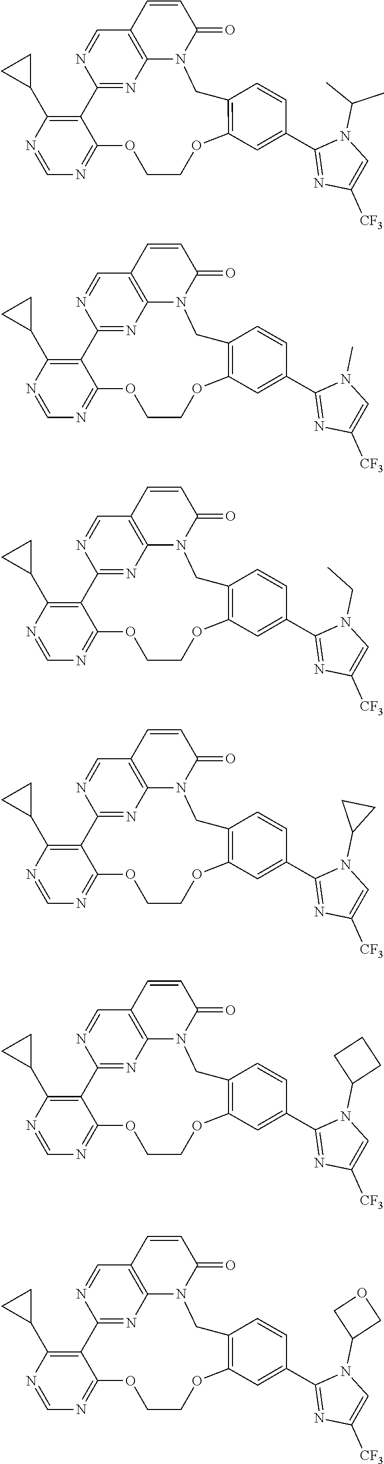 [SMILES: CC(C)n1cc(C(F)(F)F)nc1-c1ccc2c(c1)OCCOc1ncnc(C3CC3)c1-c1ncc3ccc(=O)n(c3n1)C2 ; Cn1cc(C(F)(F)F)nc1-c1ccc2c(c1)OCCOc1ncnc(C3CC3)c1-c1ncc3ccc(=O)n(c3n1)C2 ; CCn1cc(C(F)(F)F)nc1-c1ccc2c(c1)OCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCOc1ncnc(C2CC2)c1-3 ; O=c1ccc2cnc3nc2n1Cc1ccc(-c2nc(C(F)(F)F)cn2C2CCC2)cc1OCCOc1ncnc(C2CC2)c1-3 ; O=c1ccc2cnc3nc2n1Cc1ccc(-c2nc(C(F)(F)F)cn2C2COC2)cc1OCCOc1ncnc(C2CC2)c1-3]

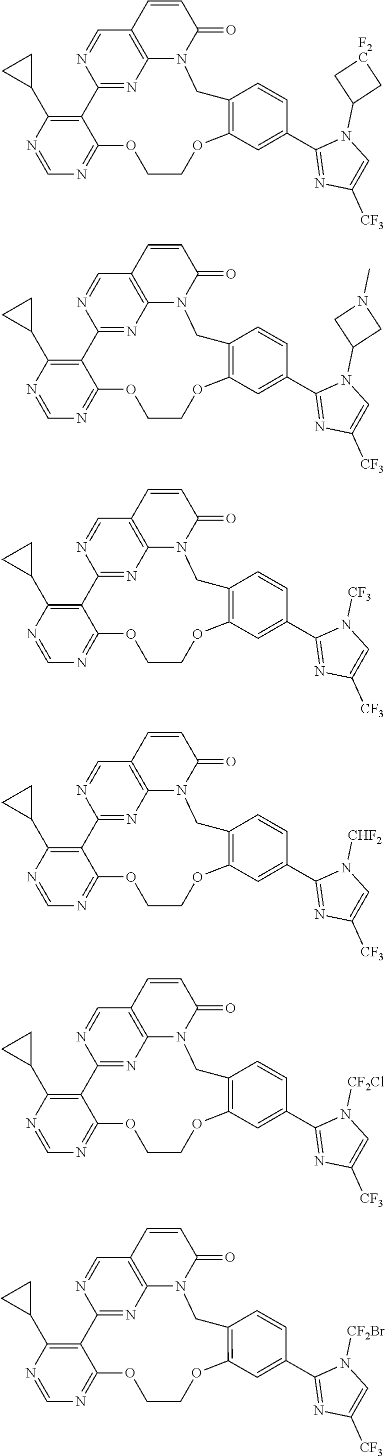

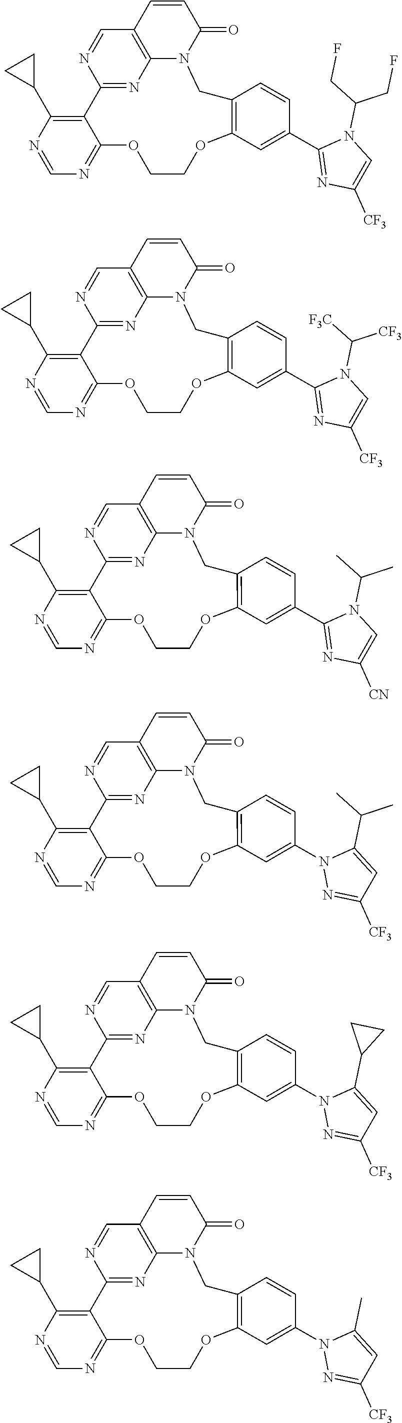 [SMILES: O=c1ccc2cnc3nc2n1Cc1ccc(-c2nc(C(F)(F)F)cn2C(CF)CF)cc1OCCOc1ncnc(C2CC2)c1-3 ; O=c1ccc2cnc3nc2n1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3ccc(=O)n(c3n1)C2 ; CC(C)c1cc(C(F)(F)F)nn1-c1ccc2c(c1)OCCOc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3ccc(=O)n(c3n1)C2]

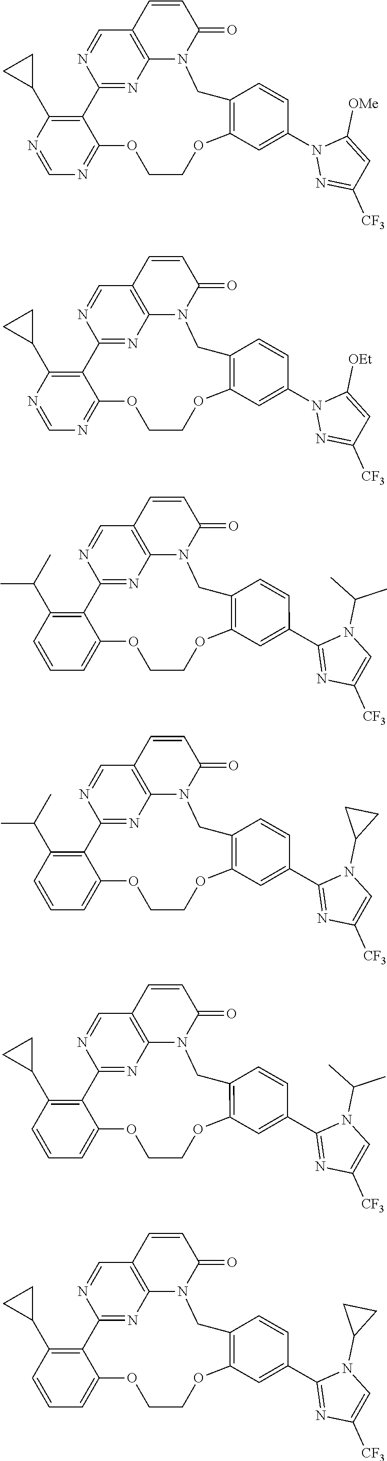

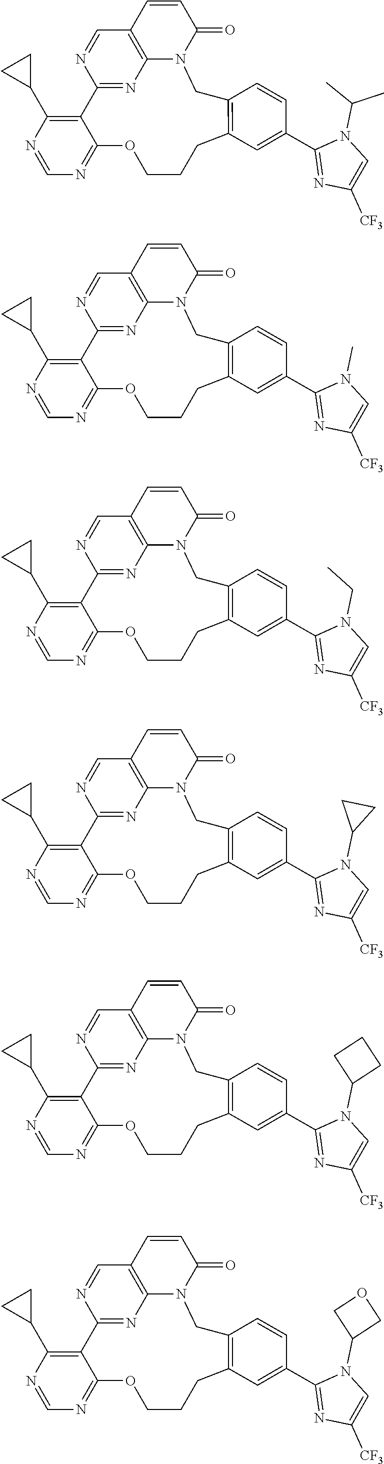 [SMILES: CC(C)n1cc(C(F)(F)F)nc1-c1ccc2c(c1)CCCOc1ncnc(C3CC3)c1-c1ncc3ccc(=O)n(c3n1)C2 ; Cn1cc(C(F)(F)F)nc1-c1ccc2c(c1)CCCOc1ncnc(C3CC3)c1-c1ncc3ccc(=O)n(c3n1)C2 ; CCn1cc(C(F)(F)F)nc1-c1ccc2c(c1)CCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCOc1ncnc(C2CC2)c1-3 ; O=c1ccc2cnc3nc2n1Cc1ccc(-c2nc(C(F)(F)F)cn2C2CCC2)cc1CCCOc1ncnc(C2CC2)c1-3 ; O=c1ccc2cnc3nc2n1Cc1ccc(-c2nc(C(F)(F)F)cn2C2COC2)cc1CCCOc1ncnc(C2CC2)c1-3]

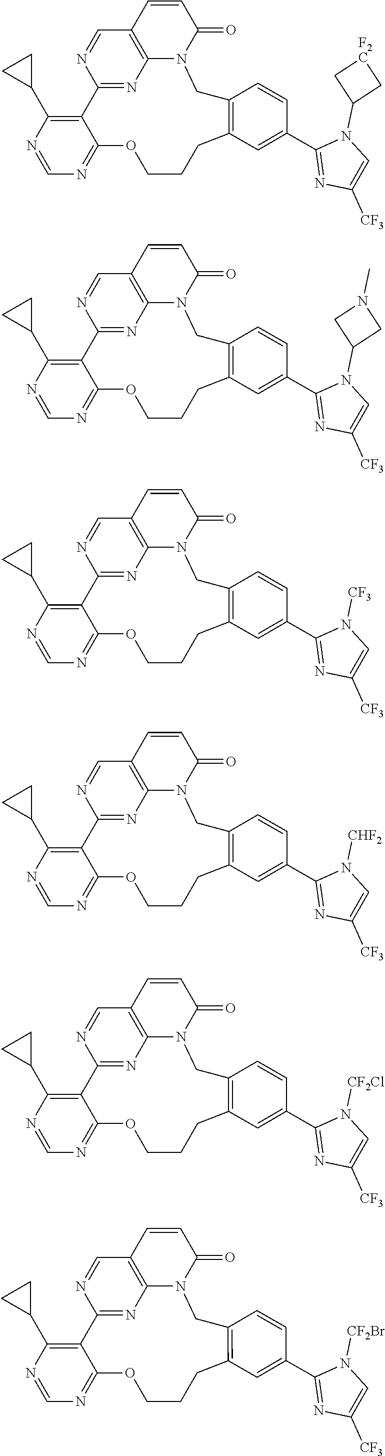

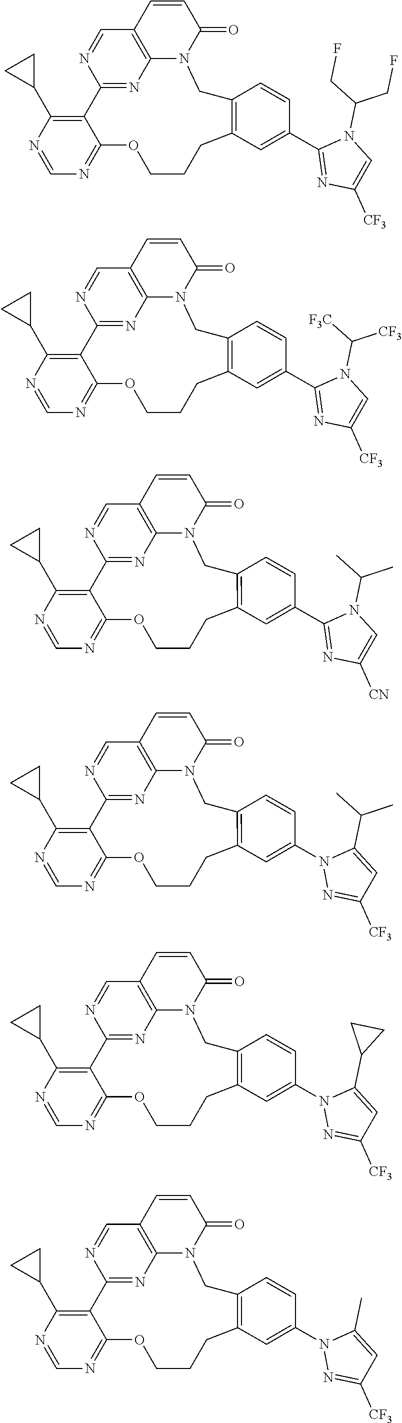 [SMILES: O=c1ccc2cnc3nc2n1Cc1ccc(-c2nc(C(F)(F)F)cn2C(CF)CF)cc1CCCOc1ncnc(C2CC2)c1-3 ; O=c1ccc2cnc3nc2n1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3ccc(=O)n(c3n1)C2 ; CC(C)c1cc(C(F)(F)F)nn1-c1ccc2c(c1)CCCOc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3ccc(=O)n(c3n1)C2]

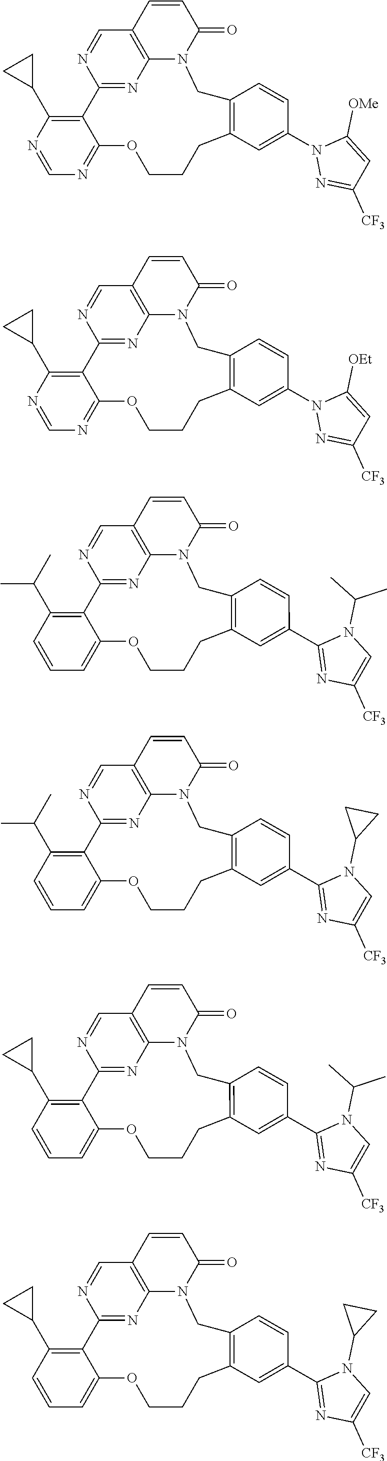

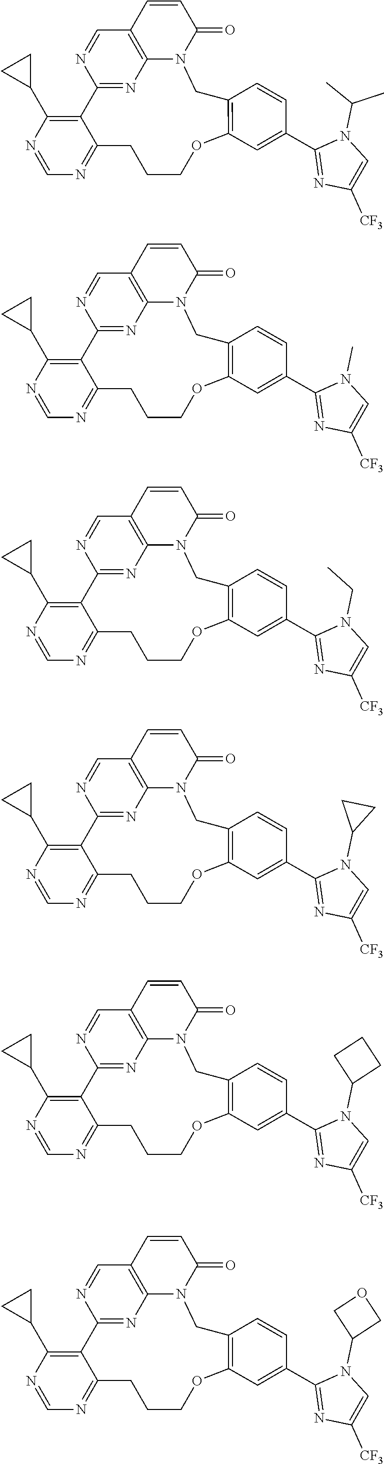 [SMILES: CC(C)n1cc(C(F)(F)F)nc1-c1ccc2c(c1)OCCCc1ncnc(C3CC3)c1-c1ncc3ccc(=O)n(c3n1)C2 ; Cn1cc(C(F)(F)F)nc1-c1ccc2c(c1)OCCCc1ncnc(C3CC3)c1-c1ncc3ccc(=O)n(c3n1)C2 ; CCn1cc(C(F)(F)F)nc1-c1ccc2c(c1)OCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCCc1ncnc(C2CC2)c1-3 ; O=c1ccc2cnc3nc2n1Cc1ccc(-c2nc(C(F)(F)F)cn2C2CCC2)cc1OCCCc1ncnc(C2CC2)c1-3 ; O=c1ccc2cnc3nc2n1Cc1ccc(-c2nc(C(F)(F)F)cn2C2COC2)cc1OCCCc1ncnc(C2CC2)c1-3]

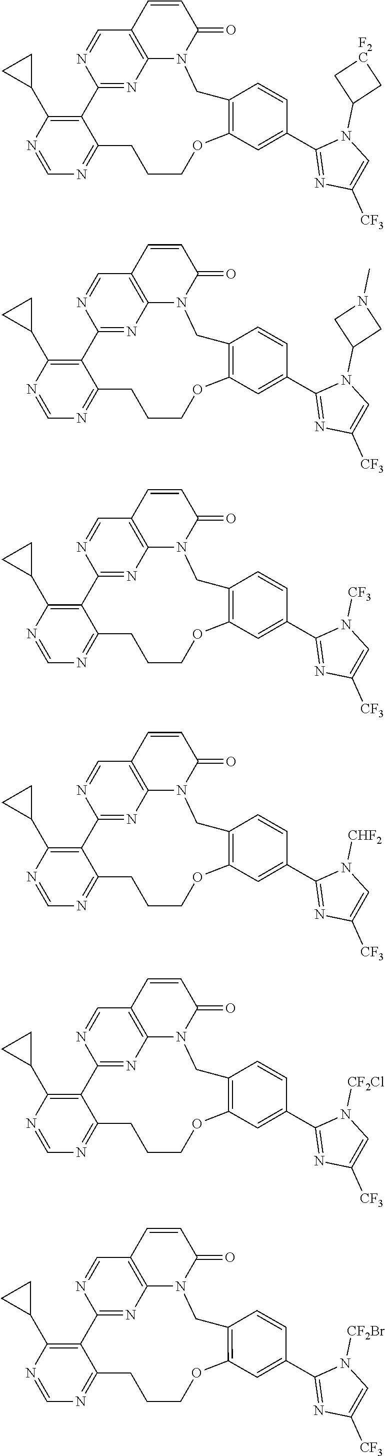

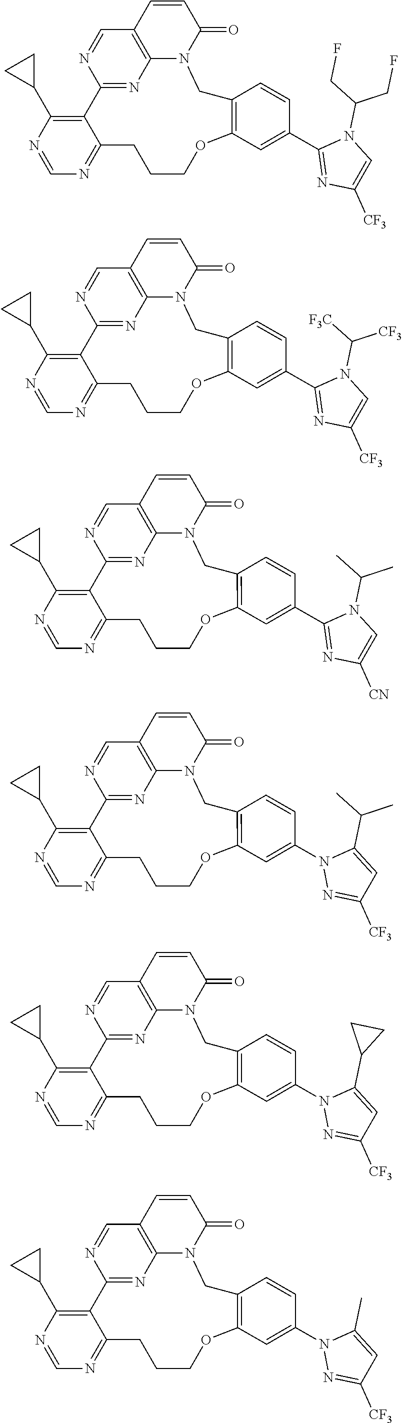 [SMILES: O=c1ccc2cnc3nc2n1Cc1ccc(-c2nc(C(F)(F)F)cn2C(CF)CF)cc1OCCCc1ncnc(C2CC2)c1-3 ; O=c1ccc2cnc3nc2n1Cc1ccc(-c2nc(C(F)(F)F)cn2C(C(F)(F)F)C(F)(F)F)cc1OCCCc1ncnc(C2CC2)c1-3 ; CC(C)n1cc(C#N)nc1-c1ccc2c(c1)OCCCc1ncnc(C3CC3)c1-c1ncc3ccc(=O)n(c3n1)C2 ; CC(C)c1cc(C(F)(F)F)nn1-c1ccc2c(c1)OCCCc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1OCCCc1ncnc(C2CC2)c1-3 ; Cc1cc(C(F)(F)F)nn1-c1ccc2c(c1)OCCCc1ncnc(C3CC3)c1-c1ncc3ccc(=O)n(c3n1)C2]

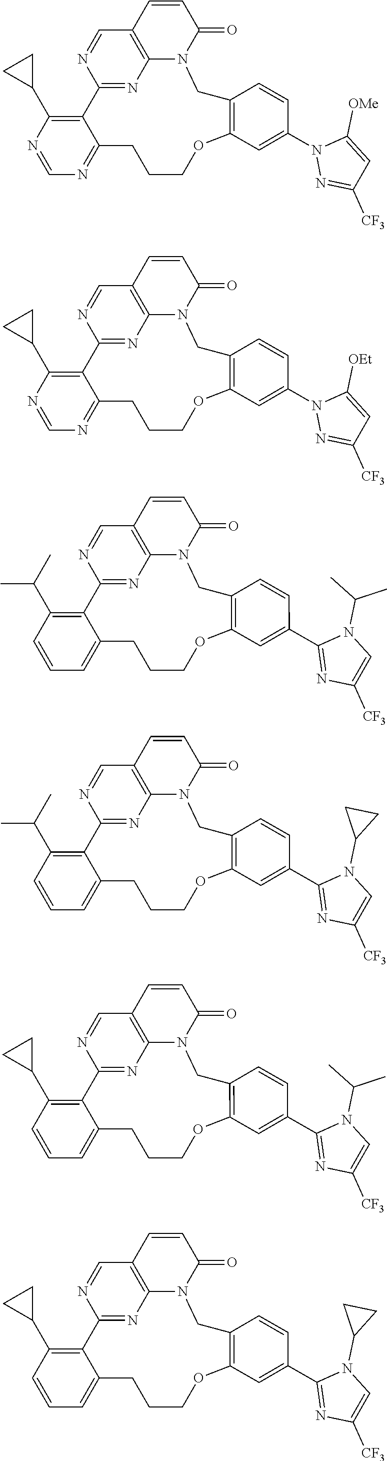

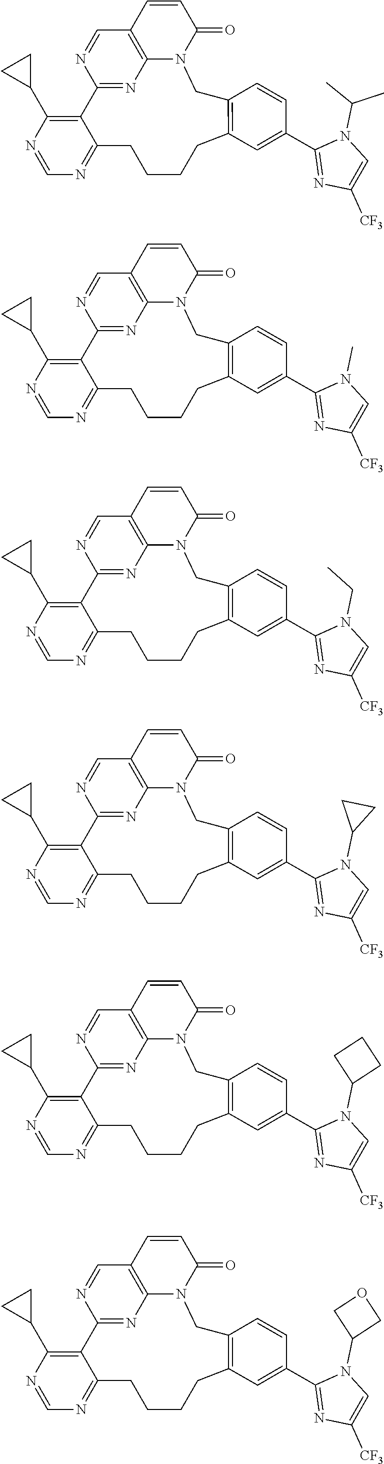 [SMILES: CC(C)n1cc(C(F)(F)F)nc1-c1ccc2c(c1)CCCCc1ncnc(C3CC3)c1-c1ncc3ccc(=O)n(c3n1)C2 ; Cn1cc(C(F)(F)F)nc1-c1ccc2c(c1)CCCCc1ncnc(C3CC3)c1-c1ncc3ccc(=O)n(c3n1)C2 ; CCn1cc(C(F)(F)F)nc1-c1ccc2c(c1)CCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCCc1ncnc(C2CC2)c1-3 ; O=c1ccc2cnc3nc2n1Cc1ccc(-c2nc(C(F)(F)F)cn2C2CCC2)cc1CCCCc1ncnc(C2CC2)c1-3 ; O=c1ccc2cnc3nc2n1Cc1ccc(-c2nc(C(F)(F)F)cn2C2COC2)cc1CCCCc1ncnc(C2CC2)c1-3]

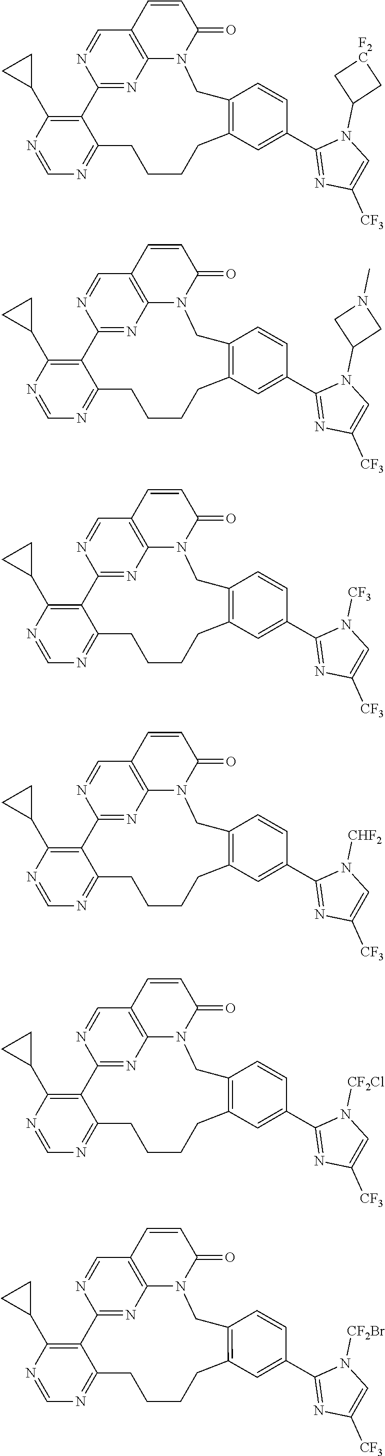

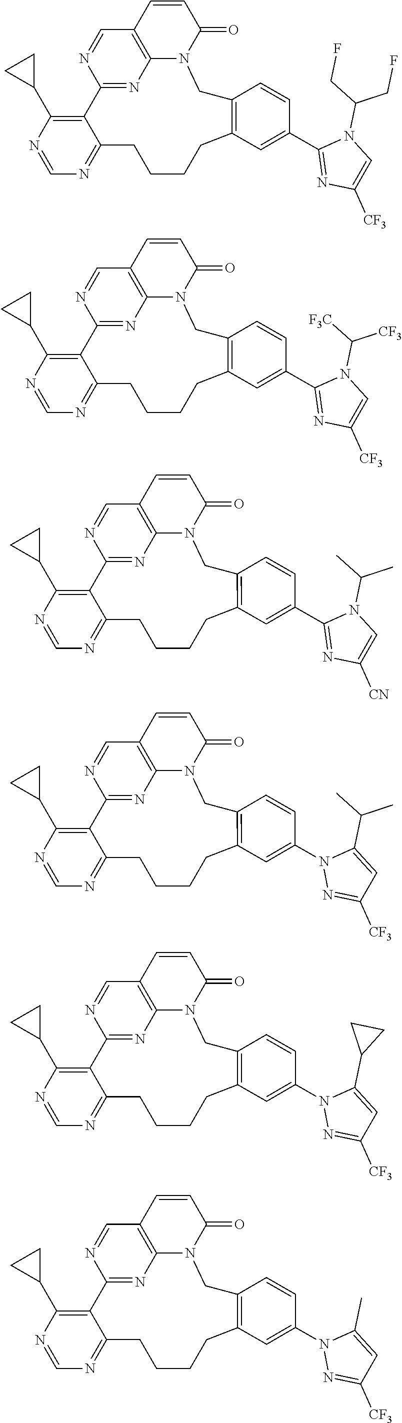 [SMILES: O=c1ccc2cnc3nc2n1Cc1ccc(-c2nc(C(F)(F)F)cn2C(CF)CF)cc1CCCCc1ncnc(C2CC2)c1-3 ; O=c1ccc2cnc3nc2n1Cc1ccc(-c2nc(C(F)(F)F)cn2C(C(F)(F)F)C(F)(F)F)cc1CCCCc1ncnc(C2CC2)c1-3 ; CC(C)n1cc(C#N)nc1-c1ccc2c(c1)CCCCc1ncnc(C3CC3)c1-c1ncc3ccc(=O)n(c3n1)C2 ; CC(C)c1cc(C(F)(F)F)nn1-c1ccc2c(c1)CCCCc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1CCCCc1ncnc(C2CC2)c1-3 ; Cc1cc(C(F)(F)F)nn1-c1ccc2c(c1)CCCCc1ncnc(C3CC3)c1-c1ncc3ccc(=O)n(c3n1)C2]

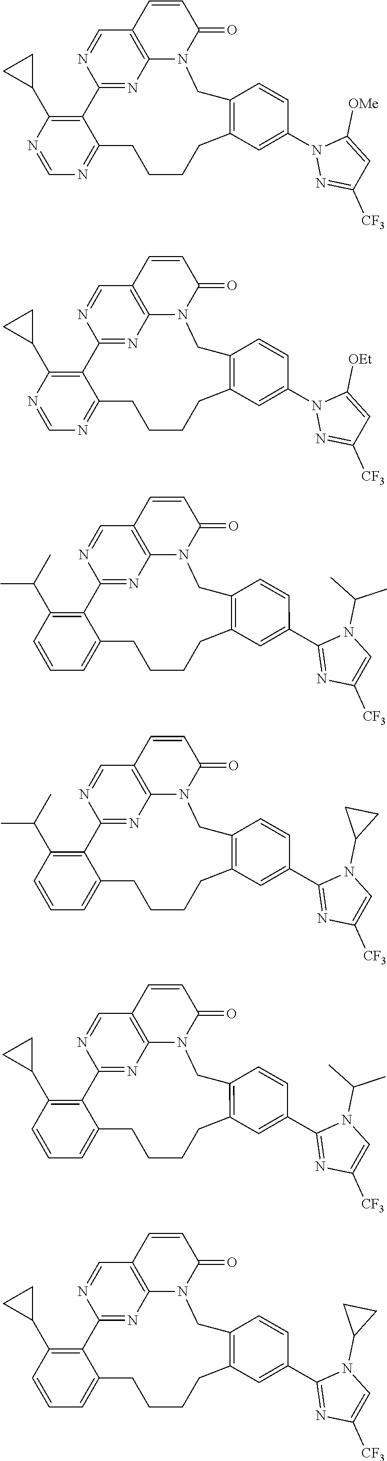

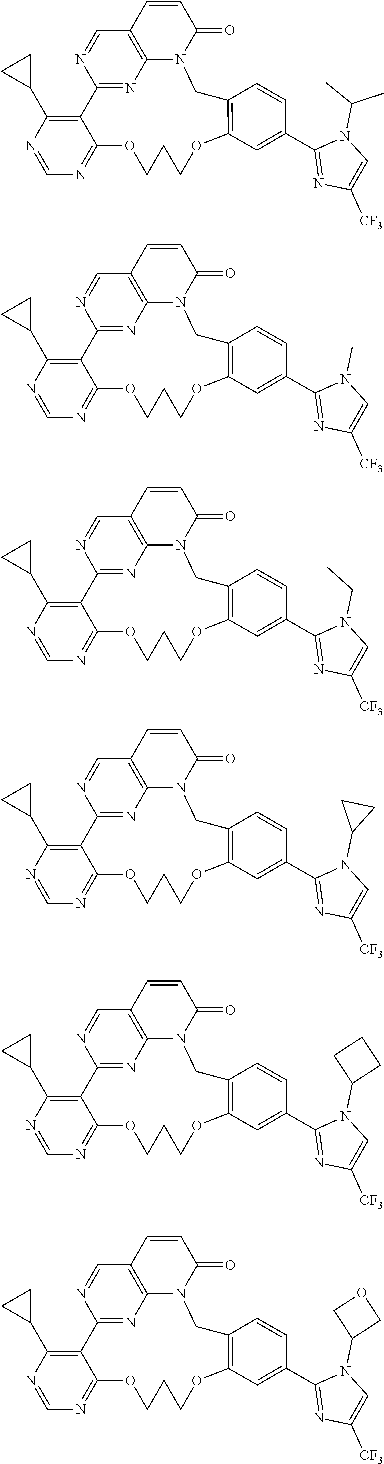 [SMILES: CC(C)n1cc(C(F)(F)F)nc1-c1ccc2c(c1)OCCCOc1ncnc(C3CC3)c1-c1ncc3ccc(=O)n(c3n1)C2 ; Cn1cc(C(F)(F)F)nc1-c1ccc2c(c1)OCCCOc1ncnc(C3CC3)c1-c1ncc3ccc(=O)n(c3n1)C2 ; CCn1cc(C(F)(F)F)nc1-c1ccc2c(c1)OCCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCCOc1ncnc(C2CC2)c1-3 ; O=c1ccc2cnc3nc2n1Cc1ccc(-c2nc(C(F)(F)F)cn2C2CCC2)cc1OCCCOc1ncnc(C2CC2)c1-3 ; O=c1ccc2cnc3nc2n1Cc1ccc(-c2nc(C(F)(F)F)cn2C2COC2)cc1OCCCOc1ncnc(C2CC2)c1-3]

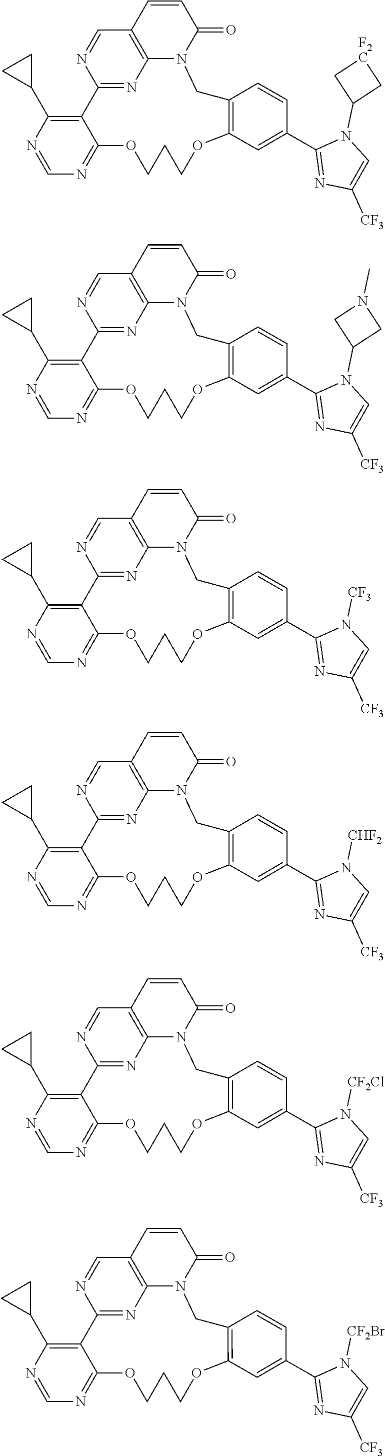

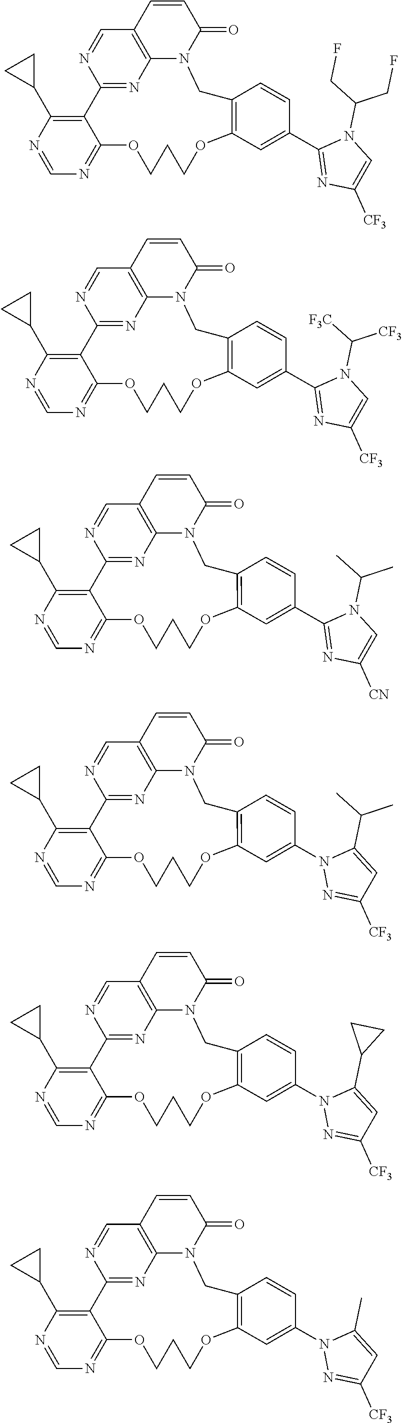 [SMILES: O=c1ccc2cnc3nc2n1Cc1ccc(-c2nc(C(F)(F)F)cn2C(CF)CF)cc1OCCCOc1ncnc(C2CC2)c1-3 ; O=c1ccc2cnc3nc2n1Cc1ccc(-c2nc(C(F)(F)F)cn2C(C(F)(F)F)C(F)(F)F)cc1OCCCOc1ncnc(C2CC2)c1-3 ; CC(C)n1cc(C#N)nc1-c1ccc2c(c1)OCCCOc1ncnc(C3CC3)c1-c1ncc3ccc(=O)n(c3n1)C2 ; CC(C)c1cc(C(F)(F)F)nn1-c1ccc2c(c1)OCCCOc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1OCCCOc1ncnc(C2CC2)c1-3 ; Cc1cc(C(F)(F)F)nn1-c1ccc2c(c1)OCCCOc1ncnc(C3CC3)c1-c1ncc3ccc(=O)n(c3n1)C2]

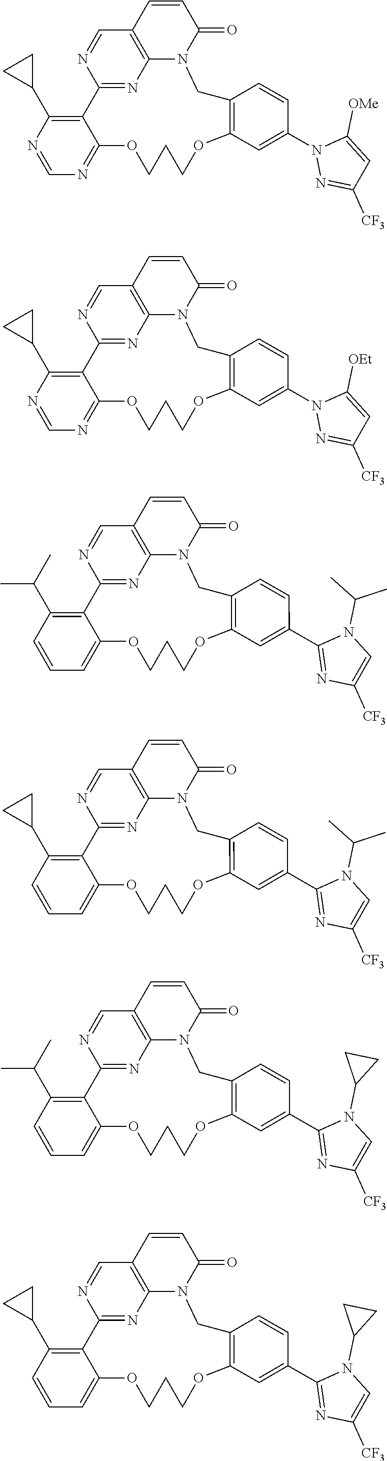

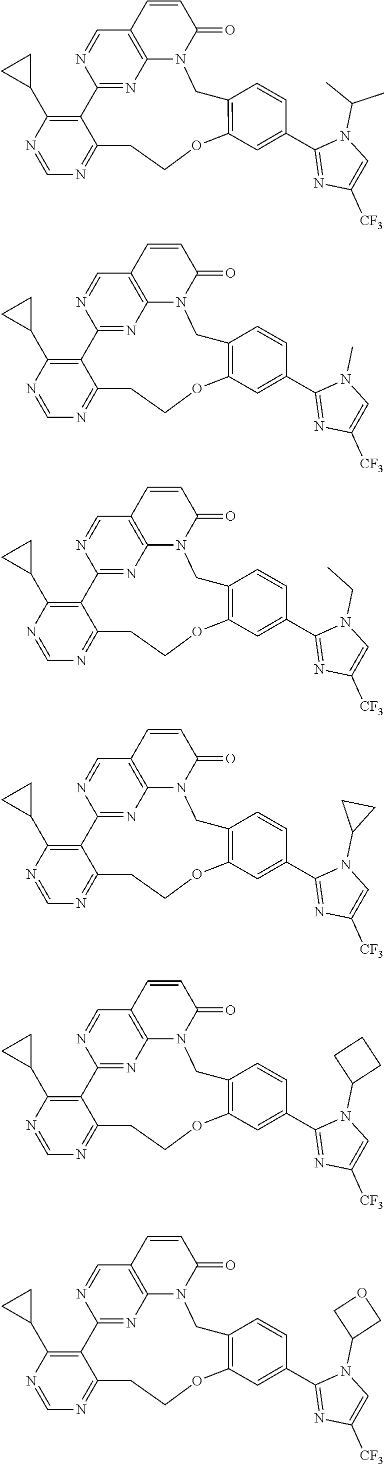 [SMILES: CC(C)n1cc(C(F)(F)F)nc1-c1ccc2c(c1)OCCc1ncnc(C3CC3)c1-c1ncc3ccc(=O)n(c3n1)C2 ; Cn1cc(C(F)(F)F)nc1-c1ccc2c(c1)OCCc1ncnc(C3CC3)c1-c1ncc3ccc(=O)n(c3n1)C2 ; CCn1cc(C(F)(F)F)nc1-c1ccc2c(c1)OCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCc1ncnc(C2CC2)c1-3 ; O=c1ccc2cnc3nc2n1Cc1ccc(-c2nc(C(F)(F)F)cn2C2CCC2)cc1OCCc1ncnc(C2CC2)c1-3 ; O=c1ccc2cnc3nc2n1Cc1ccc(-c2nc(C(F)(F)F)cn2C2COC2)cc1OCCc1ncnc(C2CC2)c1-3]

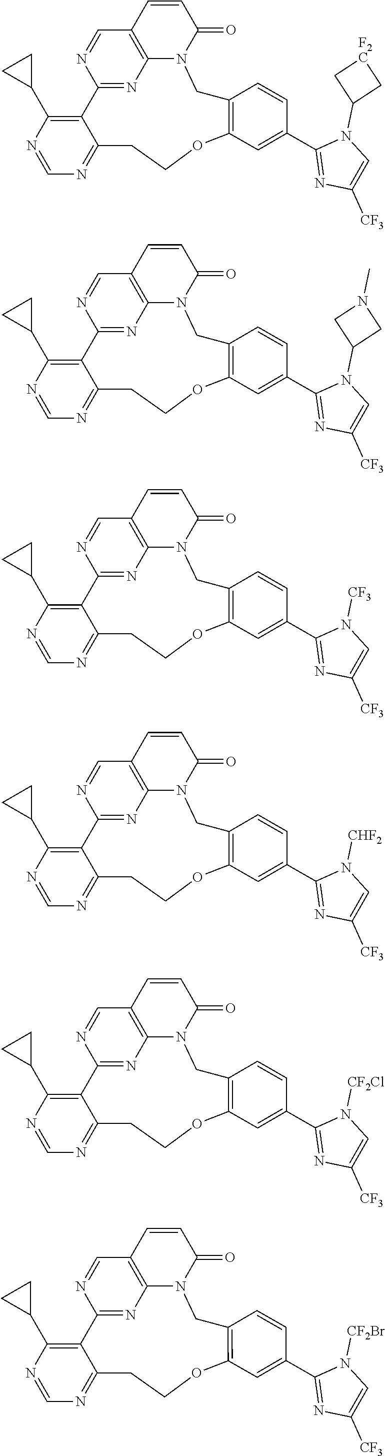

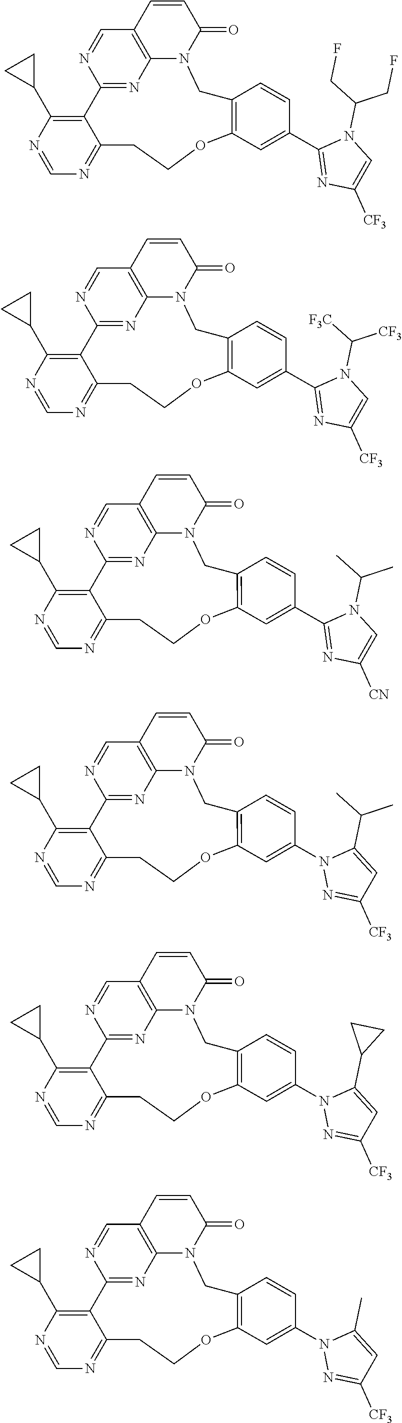 [SMILES: O=c1ccc2cnc3nc2n1Cc1ccc(-c2nc(C(F)(F)F)cn2C(CF)CF)cc1OCCc1ncnc(C2CC2)c1-3 ; O=c1ccc2cnc3nc2n1Cc1ccc(-c2nc(C(F)(F)F)cn2C(C(F)(F)F)C(F)(F)F)cc1OCCc1ncnc(C2CC2)c1-3 ; CC(C)n1cc(C#N)nc1-c1ccc2c(c1)OCCc1ncnc(C3CC3)c1-c1ncc3ccc(=O)n(c3n1)C2 ; CC(C)c1cc(C(F)(F)F)nn1-c1ccc2c(c1)OCCc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1OCCc1ncnc(C2CC2)c1-3 ; Cc1cc(C(F)(F)F)nn1-c1ccc2c(c1)OCCc1ncnc(C3CC3)c1-c1ncc3ccc(=O)n(c3n1)C2]

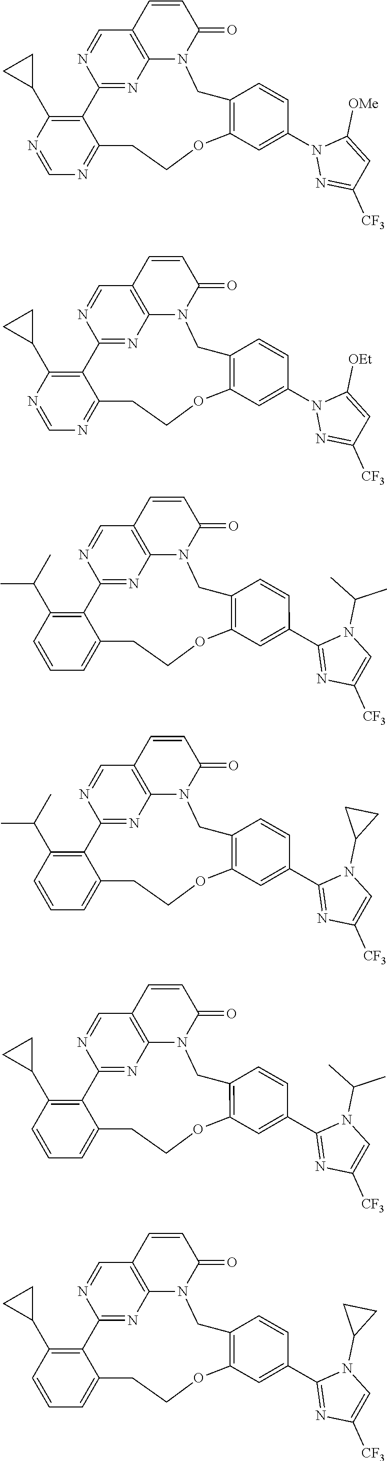

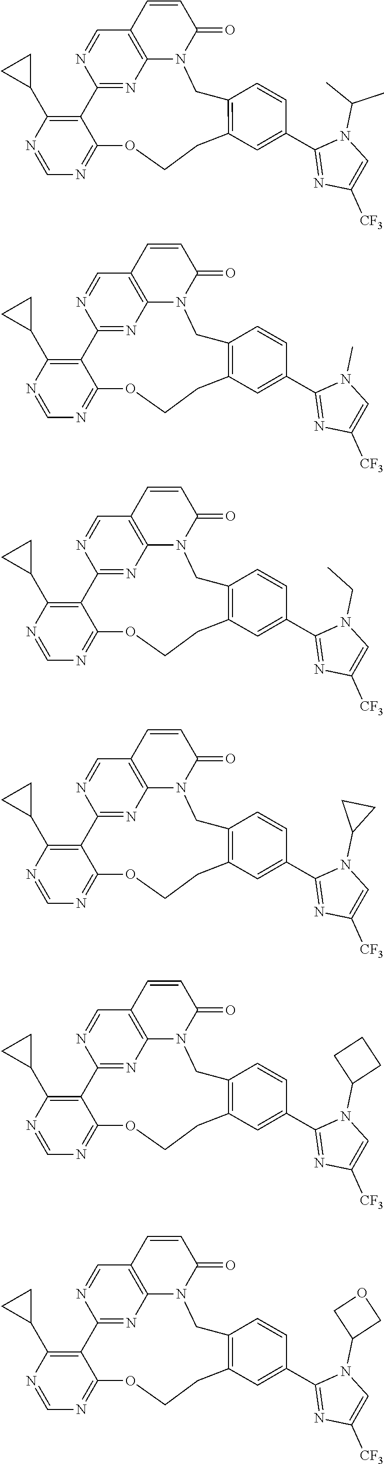 [SMILES: CC(C)n1cc(C(F)(F)F)nc1-c1ccc2c(c1)CCOc1ncnc(C3CC3)c1-c1ncc3ccc(=O)n(c3n1)C2 ; Cn1cc(C(F)(F)F)nc1-c1ccc2c(c1)CCOc1ncnc(C3CC3)c1-c1ncc3ccc(=O)n(c3n1)C2 ; CCn1cc(C(F)(F)F)nc1-c1ccc2c(c1)CCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCOc1ncnc(C2CC2)c1-3 ; O=c1ccc2cnc3nc2n1Cc1ccc(-c2nc(C(F)(F)F)cn2C2CCC2)cc1CCOc1ncnc(C2CC2)c1-3 ; O=c1ccc2cnc3nc2n1Cc1ccc(-c2nc(C(F)(F)F)cn2C2COC2)cc1CCOc1ncnc(C2CC2)c1-3]

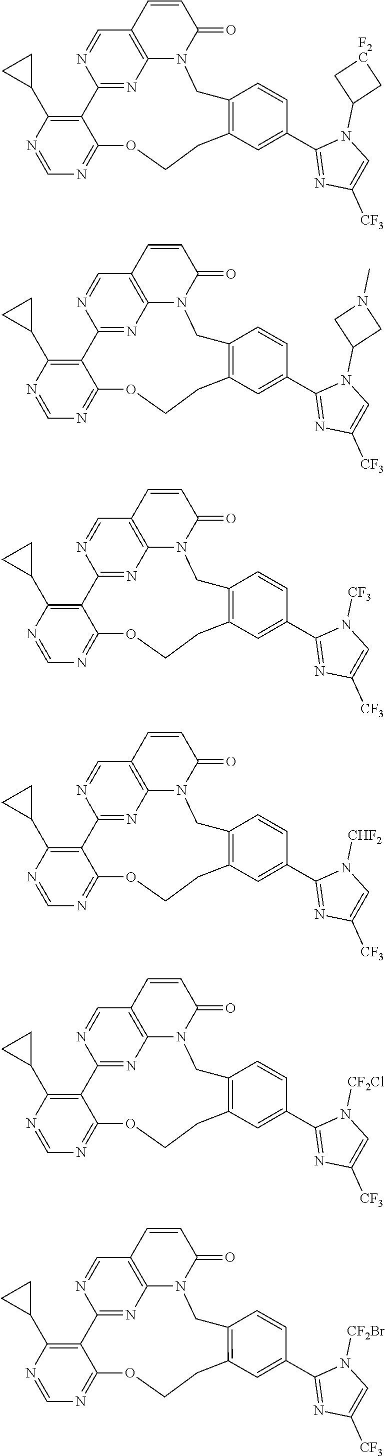

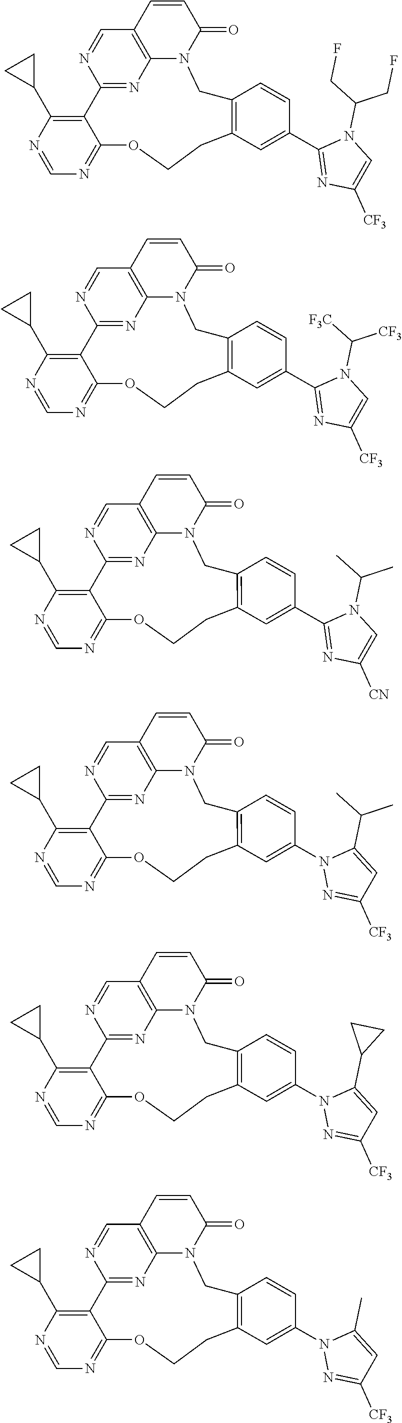 [SMILES: O=c1ccc2cnc3nc2n1Cc1ccc(-c2nc(C(F)(F)F)cn2C(CF)CF)cc1CCOc1ncnc(C2CC2)c1-3 ; O=c1ccc2cnc3nc2n1Cc1ccc(-c2nc(C(F)(F)F)cn2C(C(F)(F)F)C(F)(F)F)cc1CCOc1ncnc(C2CC2)c1-3 ; CC(C)n1cc(C#N)nc1-c1ccc2c(c1)CCOc1ncnc(C3CC3)c1-c1ncc3ccc(=O)n(c3n1)C2 ; CC(C)c1cc(C(F)(F)F)nn1-c1ccc2c(c1)CCOc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1CCOc1ncnc(C2CC2)c1-3 ; Cc1cc(C(F)(F)F)nn1-c1ccc2c(c1)CCOc1ncnc(C3CC3)c1-c1ncc3ccc(=O)n(c3n1)C2]

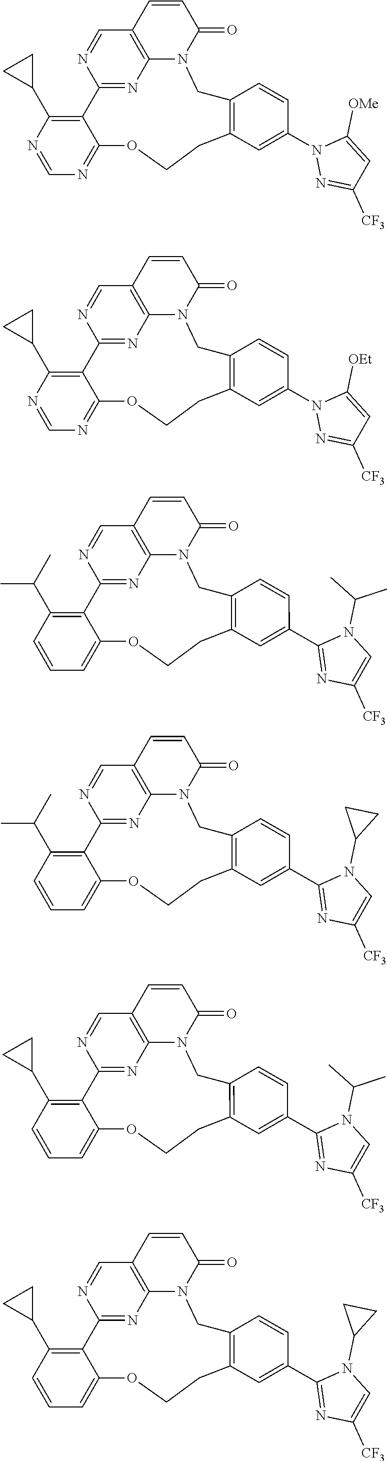

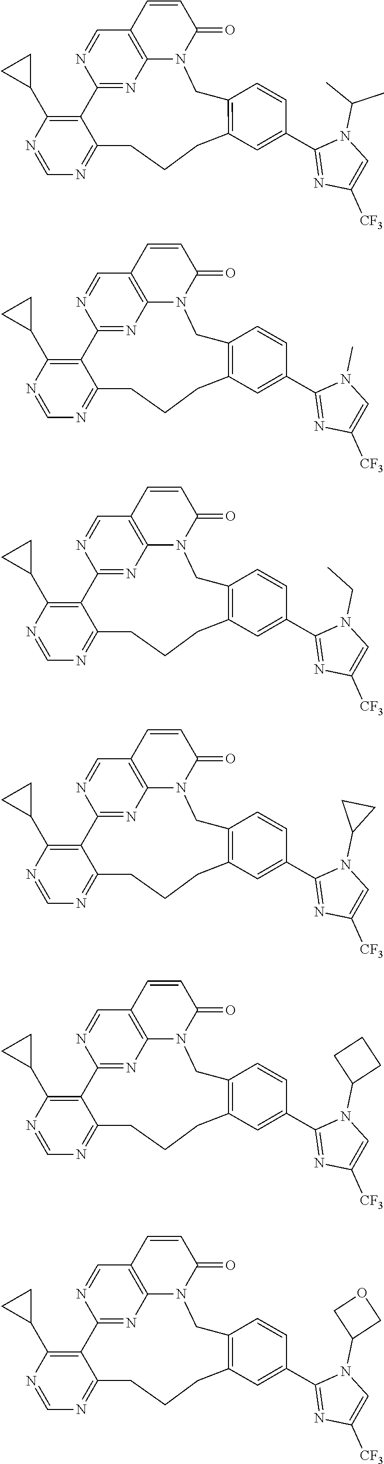 [SMILES: CC(C)n1cc(C(F)(F)F)nc1-c1ccc2c(c1)CCCc1ncnc(C3CC3)c1-c1ncc3ccc(=O)n(c3n1)C2 ; Cn1cc(C(F)(F)F)nc1-c1ccc2c(c1)CCCc1ncnc(C3CC3)c1-c1ncc3ccc(=O)n(c3n1)C2 ; CCn1cc(C(F)(F)F)nc1-c1ccc2c(c1)CCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCc1ncnc(C2CC2)c1-3 ; O=c1ccc2cnc3nc2n1Cc1ccc(-c2nc(C(F)(F)F)cn2C2CCC2)cc1CCCc1ncnc(C2CC2)c1-3 ; O=c1ccc2cnc3nc2n1Cc1ccc(-c2nc(C(F)(F)F)cn2C2COC2)cc1CCCc1ncnc(C2CC2)c1-3]

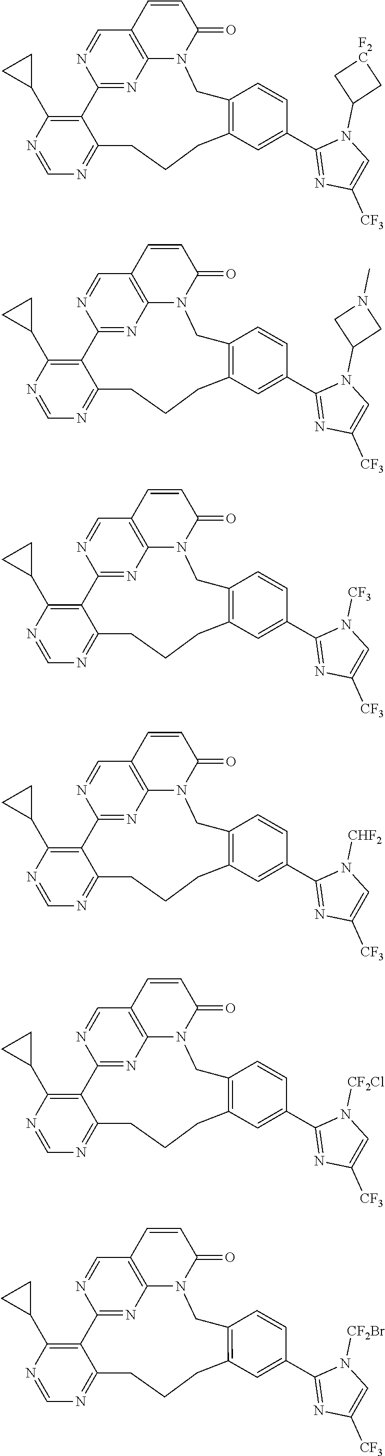

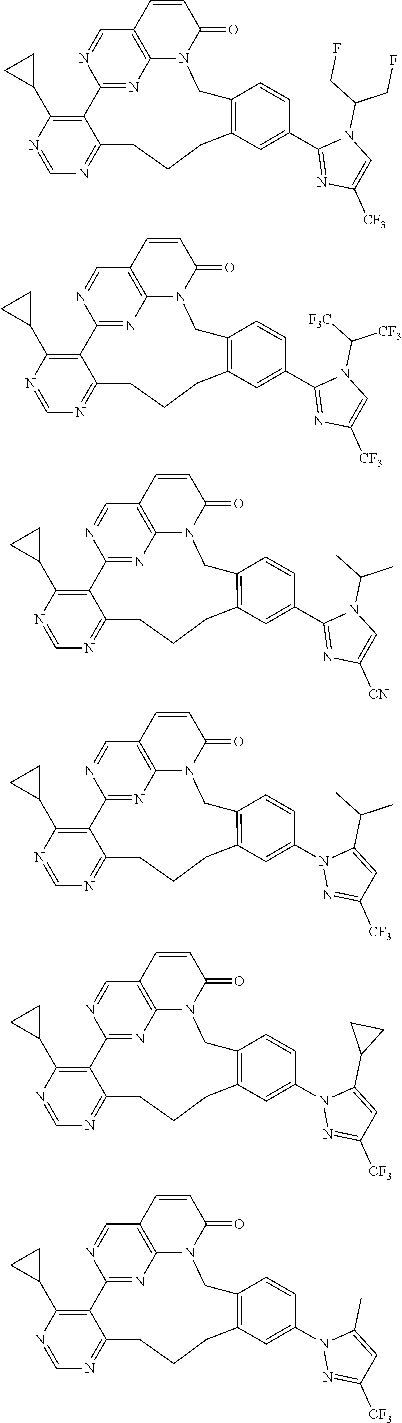 [SMILES: O=c1ccc2cnc3nc2n1Cc1ccc(-c2nc(C(F)(F)F)cn2C(CF)CF)cc1CCCc1ncnc(C2CC2)c1-3 ; O=c1ccc2cnc3nc2n1Cc1ccc(-c2nc(C(F)(F)F)cn2C(C(F)(F)F)C(F)(F)F)cc1CCCc1ncnc(C2CC2)c1-3 ; CC(C)n1cc(C#N)nc1-c1ccc2c(c1)CCCc1ncnc(C3CC3)c1-c1ncc3ccc(=O)n(c3n1)C2 ; CC(C)c1cc(C(F)(F)F)nn1-c1ccc2c(c1)CCCc1ncnc(C3CC3)c1-c1ncc3ccc(=O)n(c3n1)C2 ; O=c1ccc2cnc3nc2n1Cc1ccc(-n2nc(C(F)(F)F)cc2C2CC2)cc1CCCc1ncnc(C2CC2)c1-3 ; Cc1cc(C(F)(F)F)nn1-c1ccc2c(c1)CCCc1ncnc(C3CC3)c1-c1ncc3ccc(=O)n(c3n1)C2]

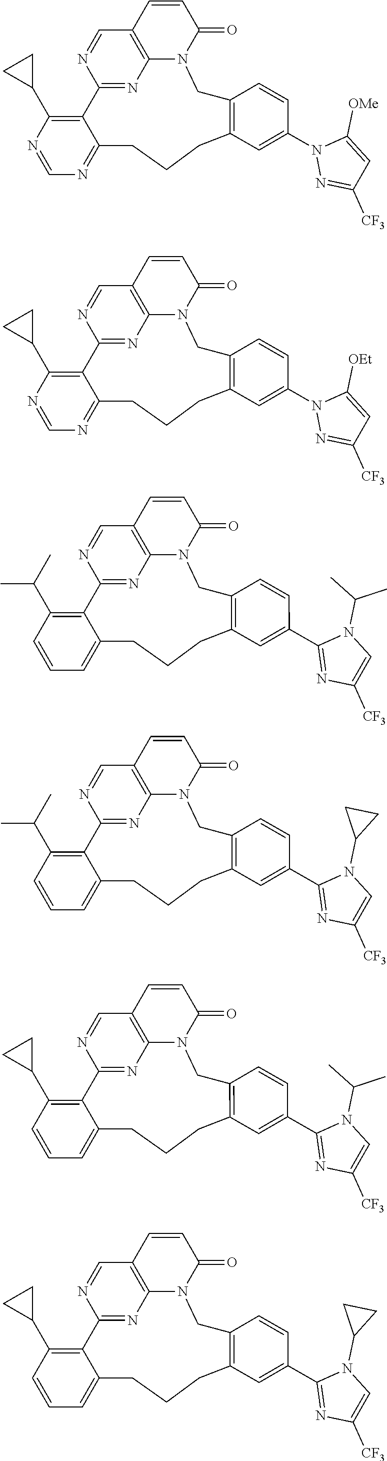

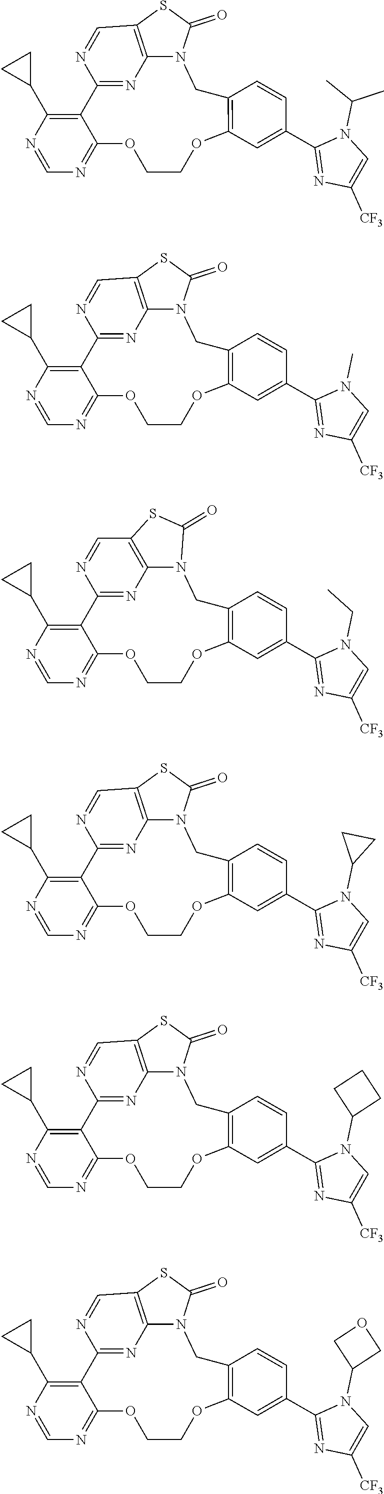 [SMILES: CC(C)n1cc(C(F)(F)F)nc1-c1ccc2c(c1)OCCOc1ncnc(C3CC3)c1-c1ncc3sc(=O)n(c3n1)C2 ; Cn1cc(C(F)(F)F)nc1-c1ccc2c(c1)OCCOc1ncnc(C3CC3)c1-c1ncc3sc(=O)n(c3n1)C2 ; CCn1cc(C(F)(F)F)nc1-c1ccc2c(c1)OCCOc1ncnc(C3CC3)c1-c1ncc3sc(=O)n(c3n1)C2 ; O=c1sc2cnc3nc2n1Cc1ccc(-c2nc(C(F)(F)F)cn2C2CC2)cc1OCCOc1ncnc(C2CC2)c1-3 ; O=c1sc2cnc3nc2n1Cc1ccc(-c2nc(C(F)(F)F)cn2C2CCC2)cc1OCCOc1ncnc(C2CC2)c1-3 ; O=c1sc2cnc3nc2n1Cc1ccc(-c2nc(C(F)(F)F)cn2C2COC2)cc1OCCOc1ncnc(C2CC2)c1-3]

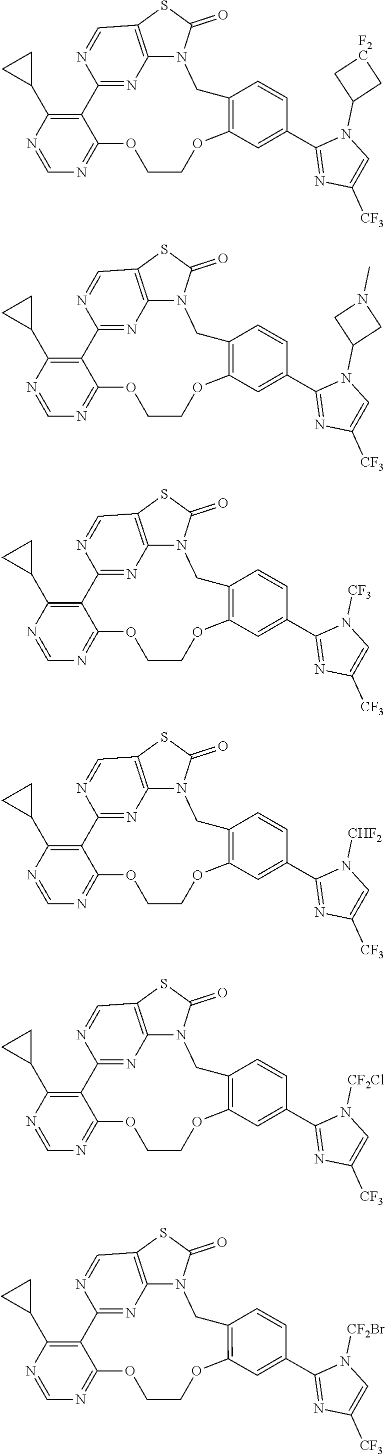

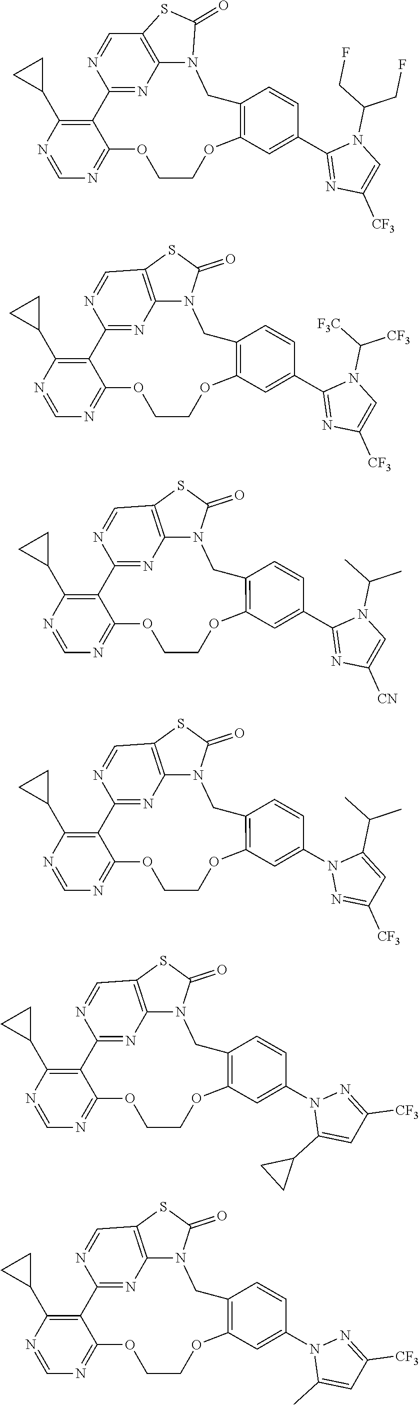 [SMILES: O=c1sc2cnc3nc2n1Cc1ccc(-c2nc(C(F)(F)F)cn2C(CF)CF)cc1OCCOc1ncnc(C2CC2)c1-3 ; O=c1sc2cnc3nc2n1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3sc(=O)n(c3n1)C2 ; CC(C)c1cc(C(F)(F)F)nn1-c1ccc2c(c1)OCCOc1ncnc(C3CC3)c1-c1ncc3sc(=O)n(c3n1)C2 ; O=c1sc2cnc3nc2n1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3sc(=O)n(c3n1)C2]

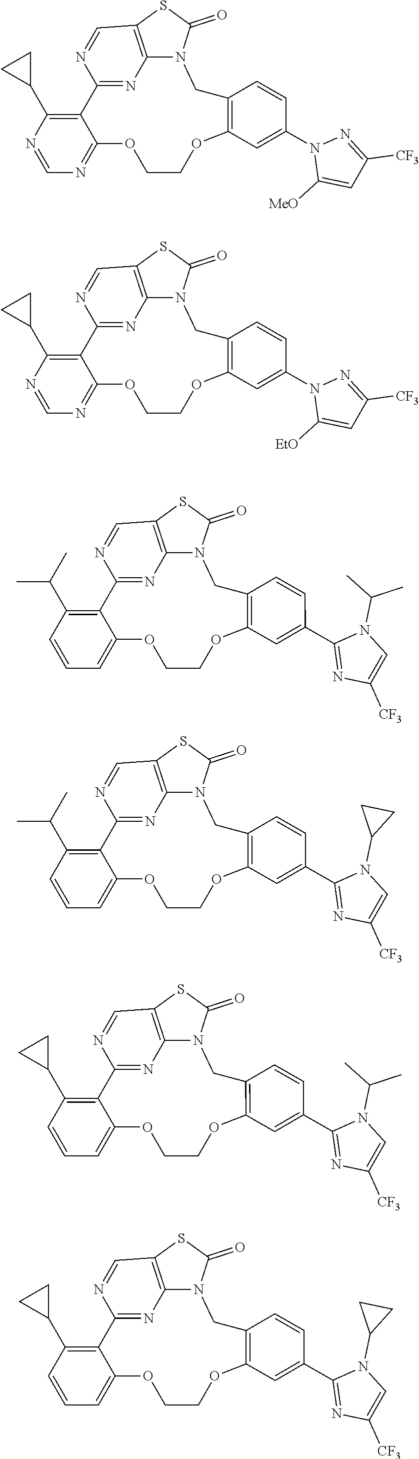

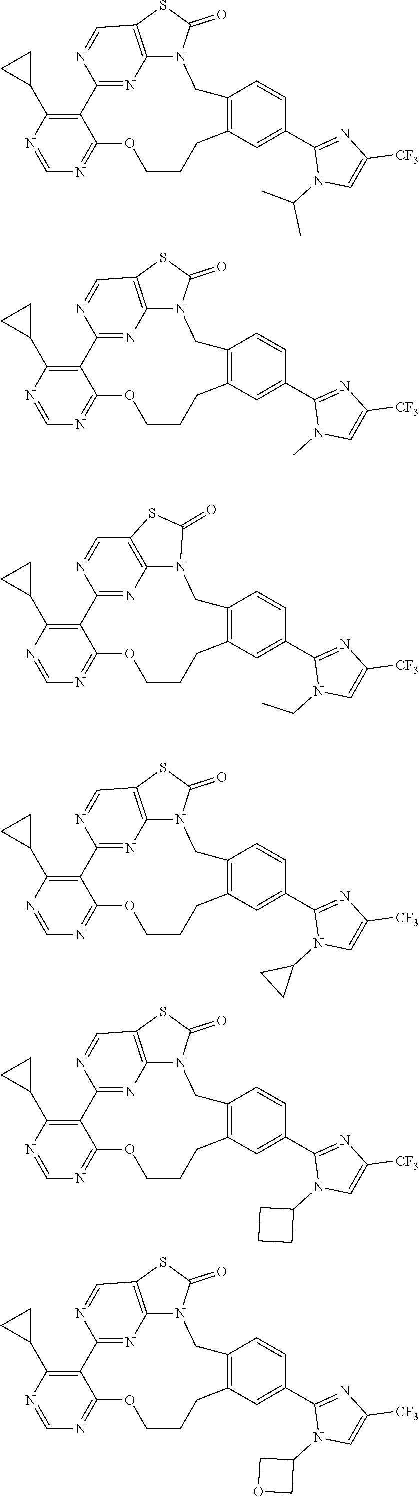 [SMILES: CC(C)n1cc(C(F)(F)F)nc1-c1ccc2c(c1)CCCOc1ncnc(C3CC3)c1-c1ncc3sc(=O)n(c3n1)C2 ; Cn1cc(C(F)(F)F)nc1-c1ccc2c(c1)CCCOc1ncnc(C3CC3)c1-c1ncc3sc(=O)n(c3n1)C2 ; CCn1cc(C(F)(F)F)nc1-c1ccc2c(c1)CCCOc1ncnc(C3CC3)c1-c1ncc3sc(=O)n(c3n1)C2 ; O=c1sc2cnc3nc2n1Cc1ccc(-c2nc(C(F)(F)F)cn2C2CC2)cc1CCCOc1ncnc(C2CC2)c1-3 ; O=c1sc2cnc3nc2n1Cc1ccc(-c2nc(C(F)(F)F)cn2C2CCC2)cc1CCCOc1ncnc(C2CC2)c1-3 ; O=c1sc2cnc3nc2n1Cc1ccc(-c2nc(C(F)(F)F)cn2C2COC2)cc1CCCOc1ncnc(C2CC2)c1-3]

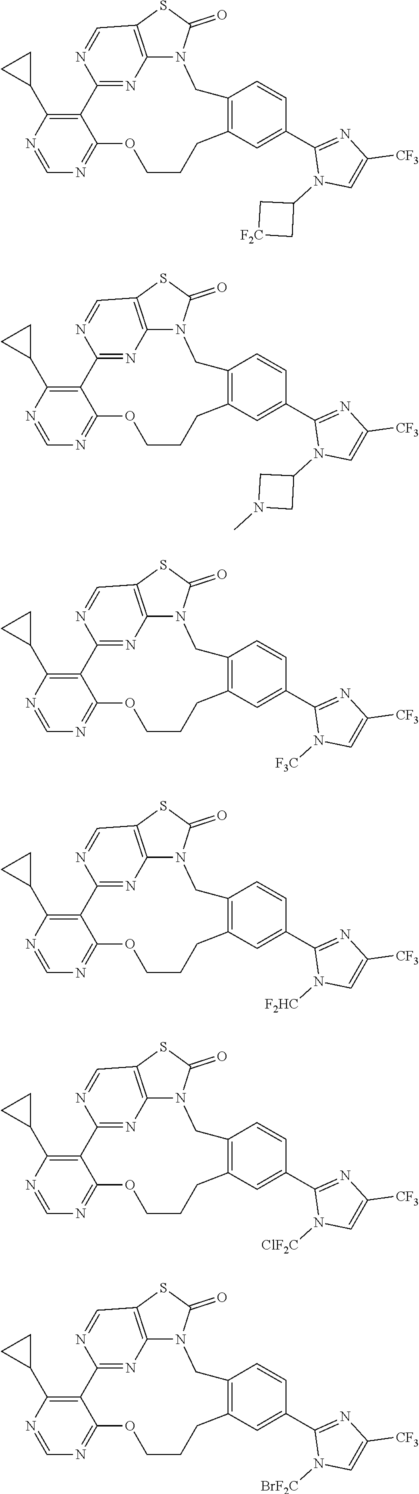

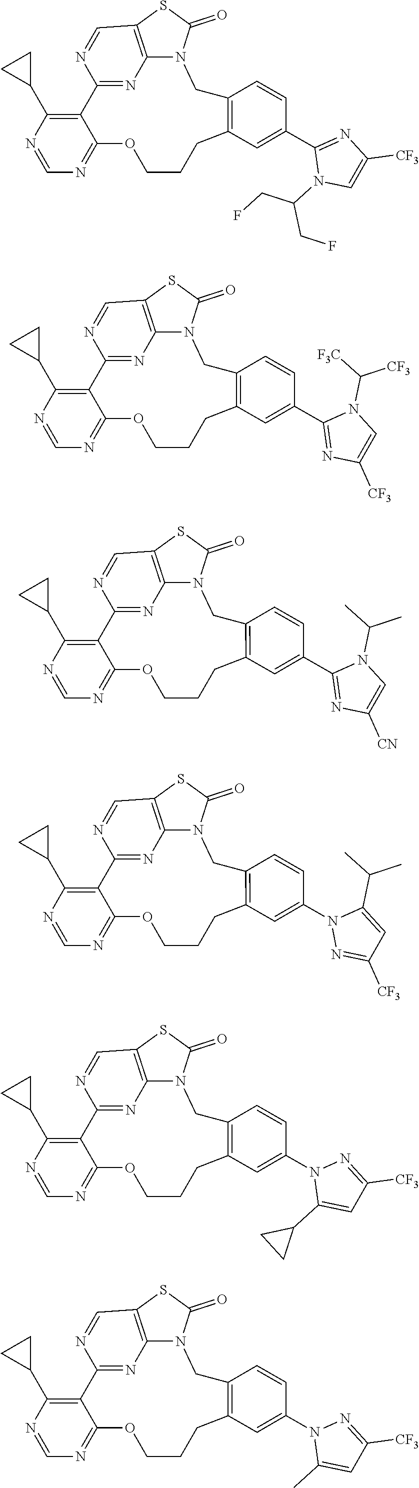 [SMILES: O=c1sc2cnc3nc2n1Cc1ccc(-c2nc(C(F)(F)F)cn2C(CF)CF)cc1CCCOc1ncnc(C2CC2)c1-3 ; O=c1sc2cnc3nc2n1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3sc(=O)n(c3n1)C2 ; CC(C)c1cc(C(F)(F)F)nn1-c1ccc2c(c1)CCCOc1ncnc(C3CC3)c1-c1ncc3sc(=O)n(c3n1)C2 ; O=c1sc2cnc3nc2n1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3sc(=O)n(c3n1)C2]

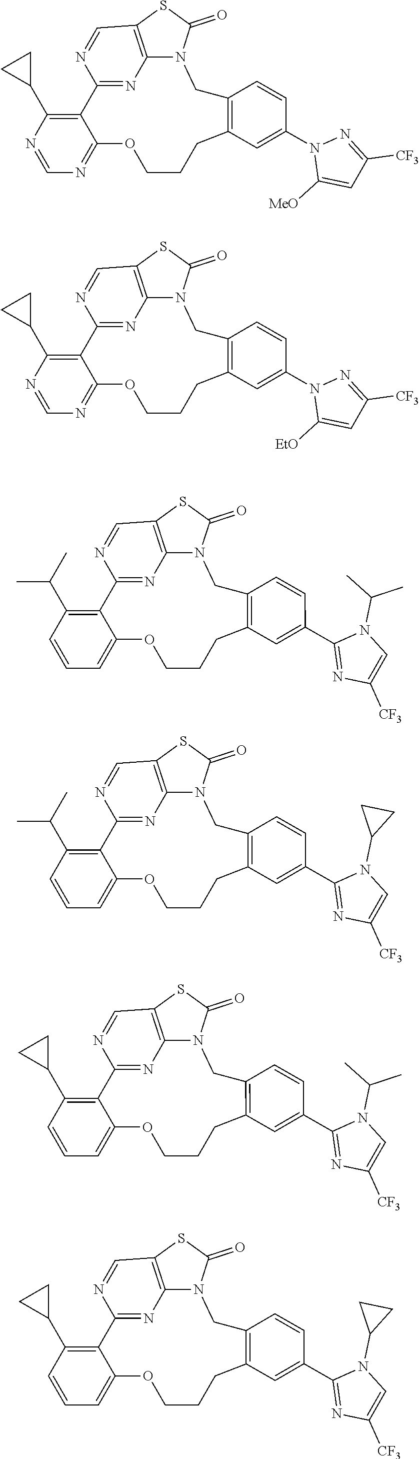

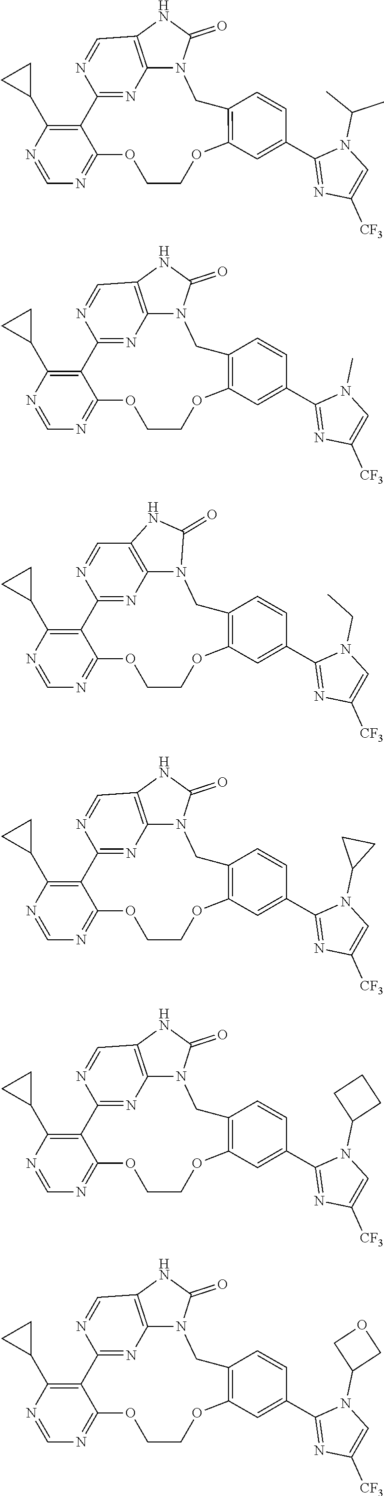 [SMILES: CC(C)n1cc(C(F)(F)F)nc1-c1ccc2c(c1)OCCOc1ncnc(C3CC3)c1-c1ncc3[nH]c(=O)n(c3n1)C2 ; Cn1cc(C(F)(F)F)nc1-c1ccc2c(c1)OCCOc1ncnc(C3CC3)c1-c1ncc3[nH]c(=O)n(c3n1)C2 ; CCn1cc(C(F)(F)F)nc1-c1ccc2c(c1)OCCOc1ncnc(C3CC3)c1-c1ncc3[nH]c(=O)n(c3n1)C2 ; O=c1[nH]c2cnc3nc2n1Cc1ccc(-c2nc(C(F)(F)F)cn2C2CC2)cc1OCCOc1ncnc(C2CC2)c1-3 ; O=c1[nH]c2cnc3nc2n1Cc1ccc(-c2nc(C(F)(F)F)cn2C2CCC2)cc1OCCOc1ncnc(C2CC2)c1-3 ; O=c1[nH]c2cnc3nc2n1Cc1ccc(-c2nc(C(F)(F)F)cn2C2COC2)cc1OCCOc1ncnc(C2CC2)c1-3]

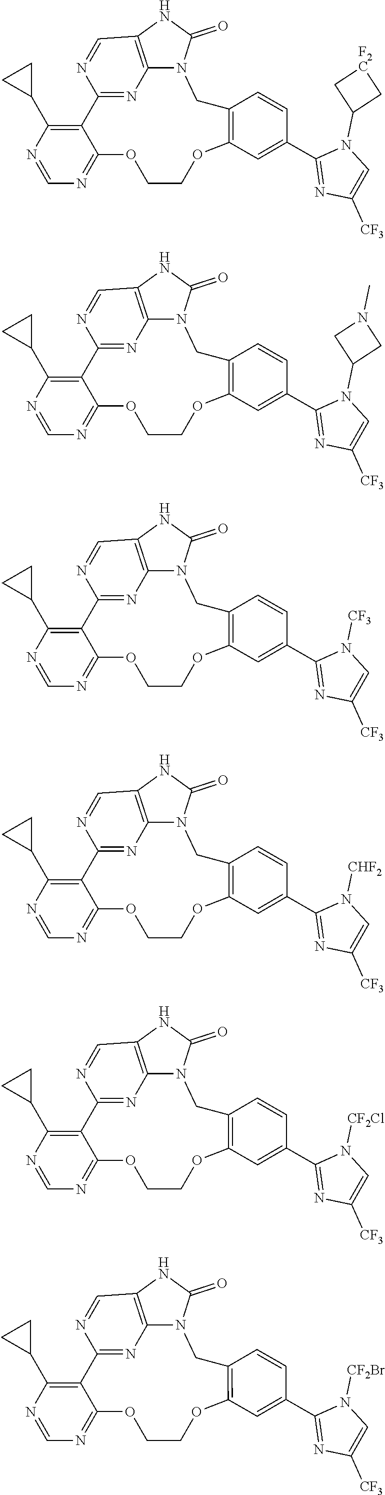

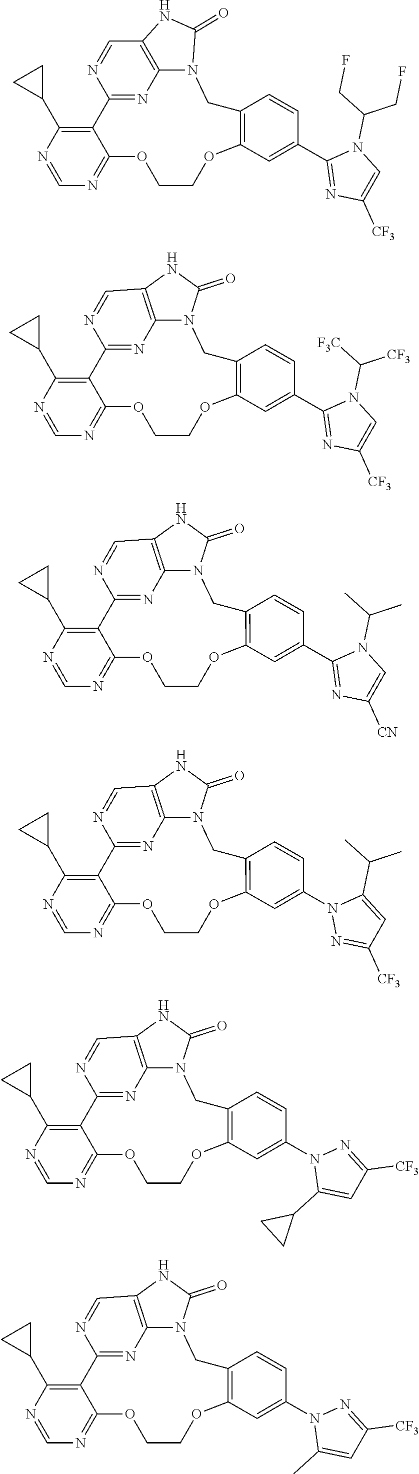 [SMILES: O=c1[nH]c2cnc3nc2n1Cc1ccc(-c2nc(C(F)(F)F)cn2C(CF)CF)cc1OCCOc1ncnc(C2CC2)c1-3 ; O=c1[nH]c2cnc3nc2n1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3[nH]c(=O)n(c3n1)C2 ; CC(C)c1cc(C(F)(F)F)nn1-c1ccc2c(c1)OCCOc1ncnc(C3CC3)c1-c1ncc3[nH]c(=O)n(c3n1)C2 ; O=c1[nH]c2cnc3nc2n1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3[nH]c(=O)n(c3n1)C2]

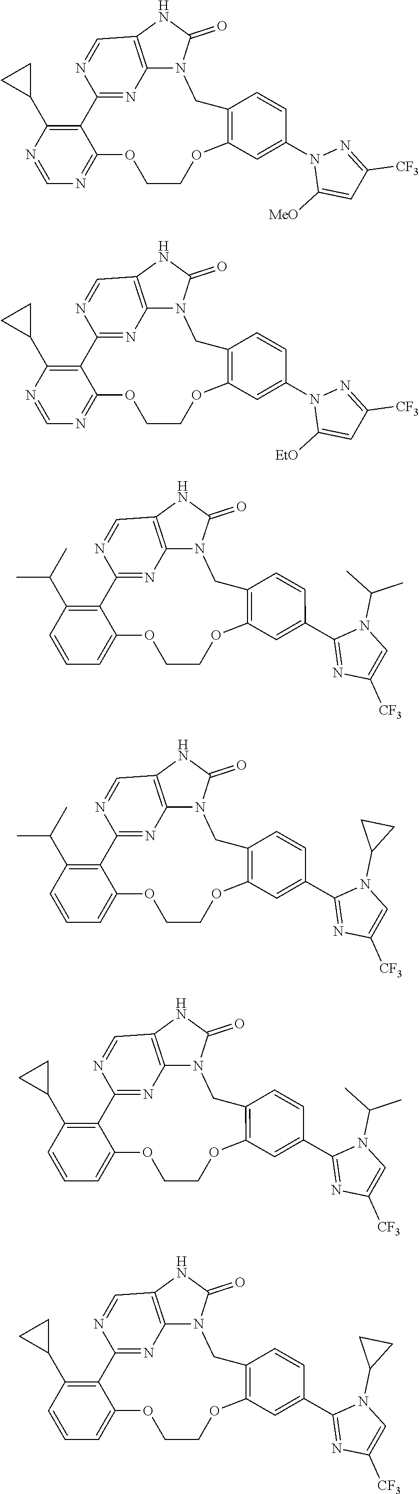

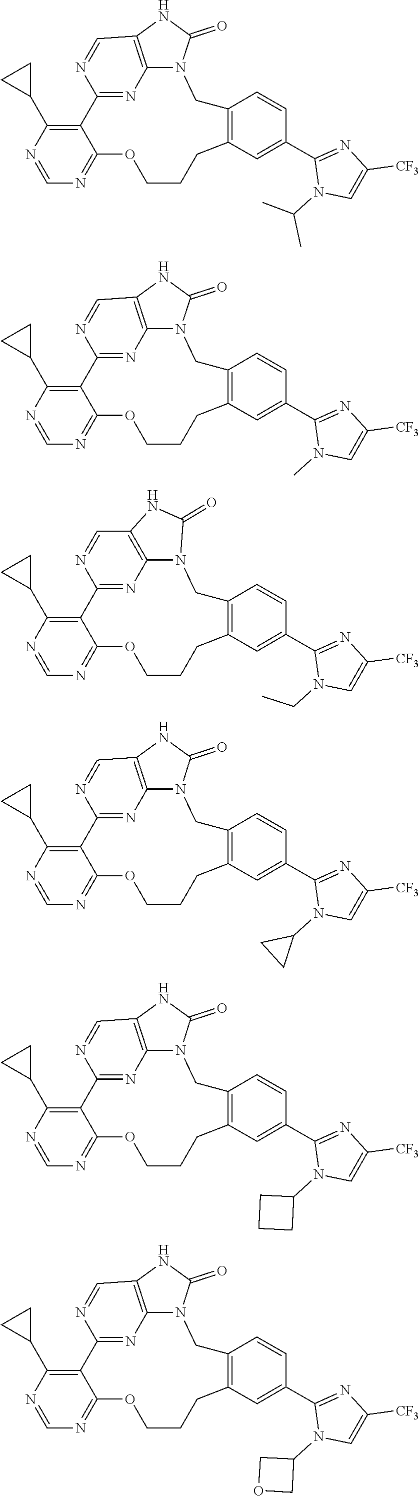 [SMILES: CC(C)n1cc(C(F)(F)F)nc1-c1ccc2c(c1)CCCOc1ncnc(C3CC3)c1-c1ncc3[nH]c(=O)n(c3n1)C2 ; Cn1cc(C(F)(F)F)nc1-c1ccc2c(c1)CCCOc1ncnc(C3CC3)c1-c1ncc3[nH]c(=O)n(c3n1)C2 ; CCn1cc(C(F)(F)F)nc1-c1ccc2c(c1)CCCOc1ncnc(C3CC3)c1-c1ncc3[nH]c(=O)n(c3n1)C2 ; O=c1[nH]c2cnc3nc2n1Cc1ccc(-c2nc(C(F)(F)F)cn2C2CC2)cc1CCCOc1ncnc(C2CC2)c1-3 ; O=c1[nH]c2cnc3nc2n1Cc1ccc(-c2nc(C(F)(F)F)cn2C2CCC2)cc1CCCOc1ncnc(C2CC2)c1-3 ; O=c1[nH]c2cnc3nc2n1Cc1ccc(-c2nc(C(F)(F)F)cn2C2COC2)cc1CCCOc1ncnc(C2CC2)c1-3]

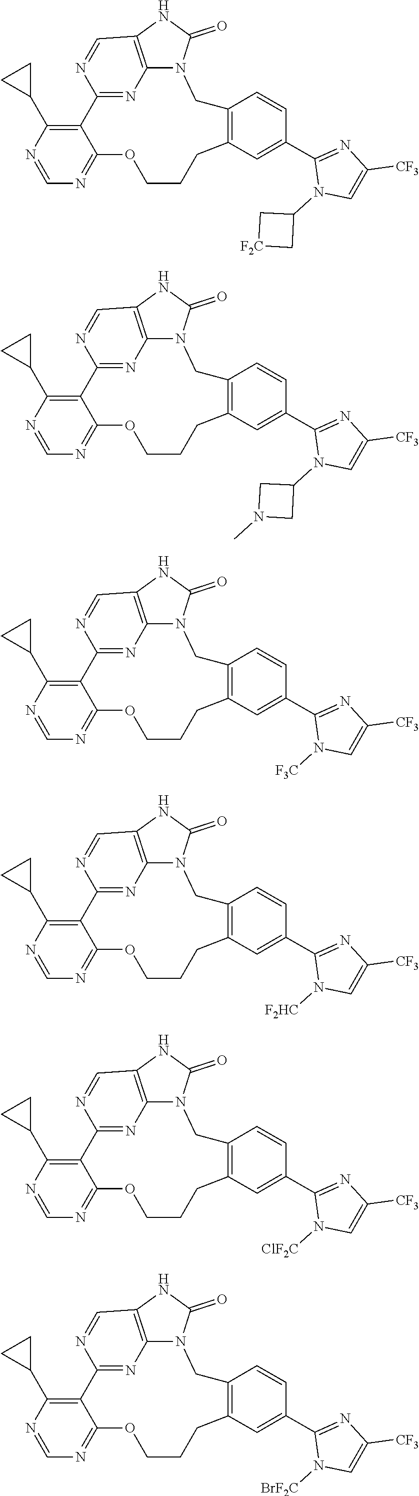

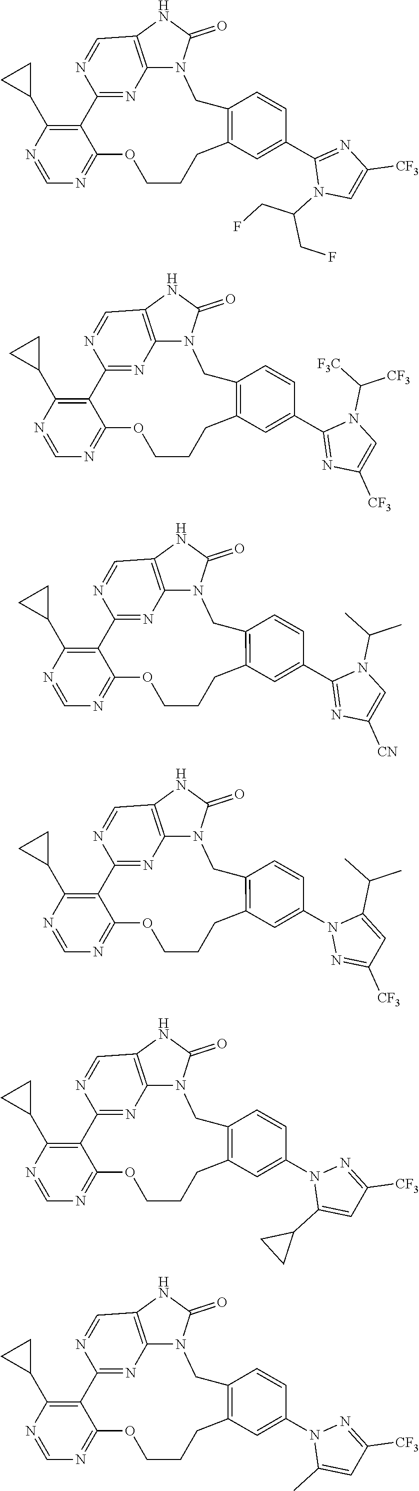 [SMILES: O=c1[nH]c2cnc3nc2n1Cc1ccc(-c2nc(C(F)(F)F)cn2C(CF)CF)cc1CCCOc1ncnc(C2CC2)c1-3 ; O=c1[nH]c2cnc3nc2n1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3[nH]c(=O)n(c3n1)C2 ; CC(C)c1cc(C(F)(F)F)nn1-c1ccc2c(c1)CCCOc1ncnc(C3CC3)c1-c1ncc3[nH]c(=O)n(c3n1)C2 ; O=c1[nH]c2cnc3nc2n1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3[nH]c(=O)n(c3n1)C2]

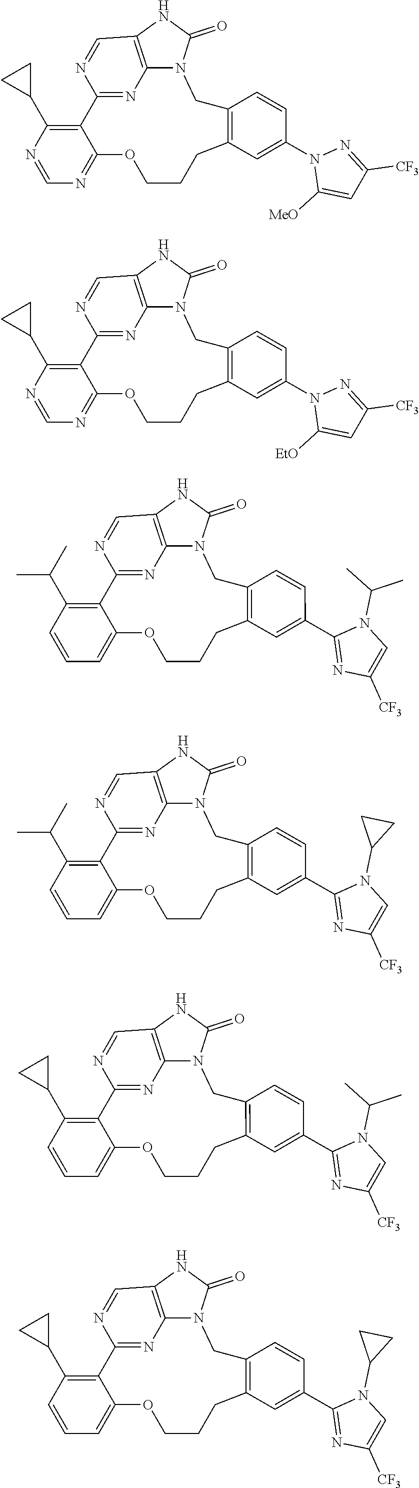

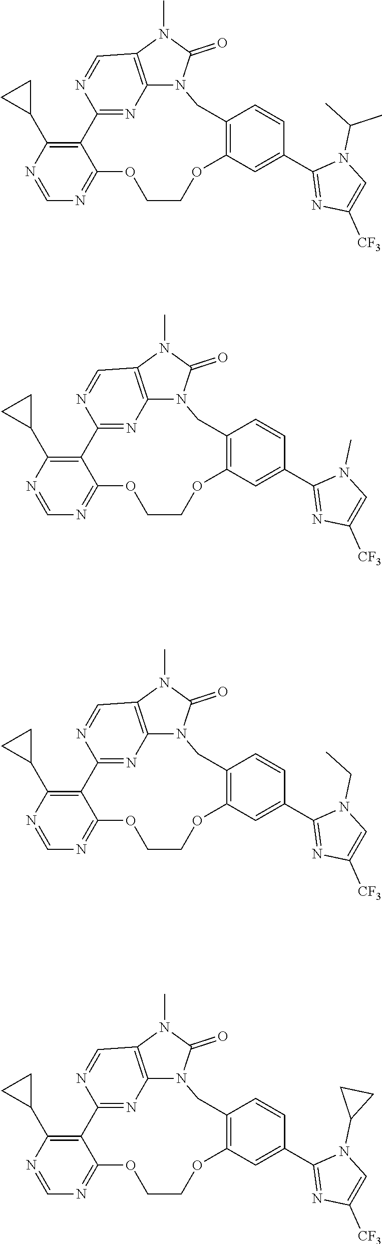 [SMILES: CC(C)n1cc(C(F)(F)F)nc1-c1ccc2c(c1)OCCOc1ncnc(C3CC3)c1-c1ncc3c(n1)n(c(=O)n3C)C2 ; Cn1cc(C(F)(F)F)nc1-c1ccc2c(c1)OCCOc1ncnc(C3CC3)c1-c1ncc3c(n1)n(c(=O)n3C)C2 ; CCn1cc(C(F)(F)F)nc1-c1ccc2c(c1)OCCOc1ncnc(C3CC3)c1-c1ncc3c(n1)n(c(=O)n3C)C2 ; Cn1c(=O)n2c3nc(ncc31)-c1c(ncnc1C1CC1)OCCOc1cc(-c3nc(C(F)(F)F)cn3C3CC3)ccc1C2]

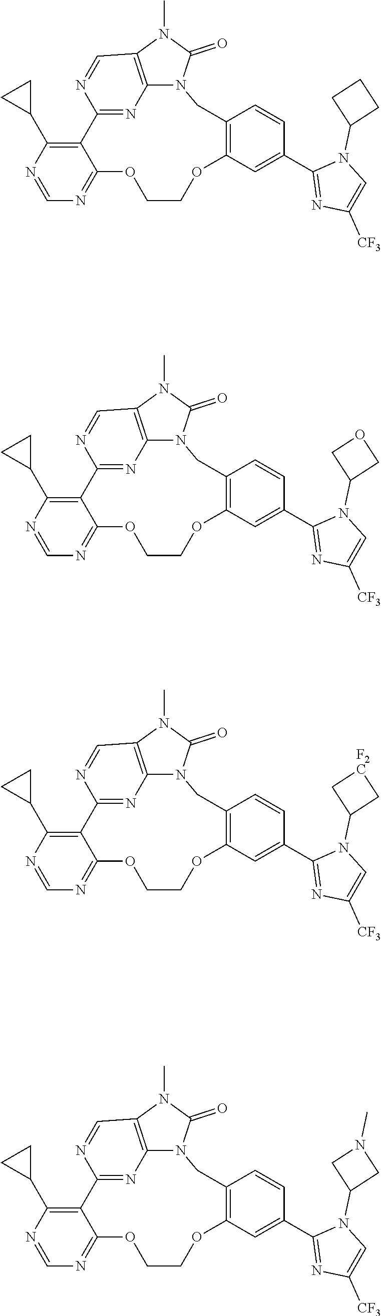 [SMILES: Cn1c(=O)n2c3nc(ncc31)-c1c(ncnc1C1CC1)OCCOc1cc(-c3nc(C(F)(F)F)cn3C3CCC3)ccc1C2 ; Cn1c(=O)n2c3nc(ncc31)-c1c(ncnc1C1CC1)OCCOc1cc(-c3nc(C(F)(F)F)cn3C3COC3)ccc1C2 ; Cn1c(=O)n2c3nc(ncc31)-c1c(ncnc1C1CC1)OCCOc1cc(-c3nc(C(F)(F)F)cn3C3CC(F)(F)C3)ccc1C2 ; CN1CC(n2cc(C(F)(F)F)nc2-c2ccc3c(c2)OCCOc2ncnc(C4CC4)c2-c2ncc4c(n2)n(c(=O)n4C)C3)C1]

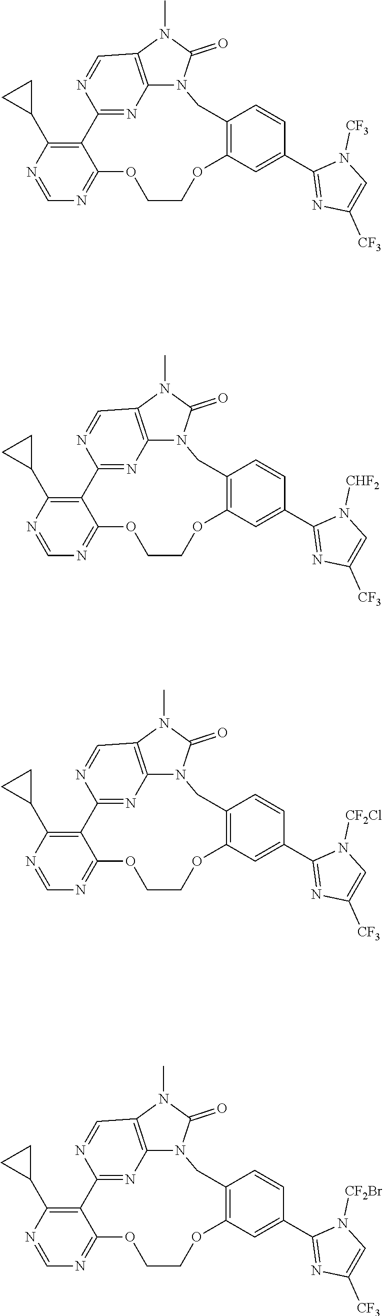

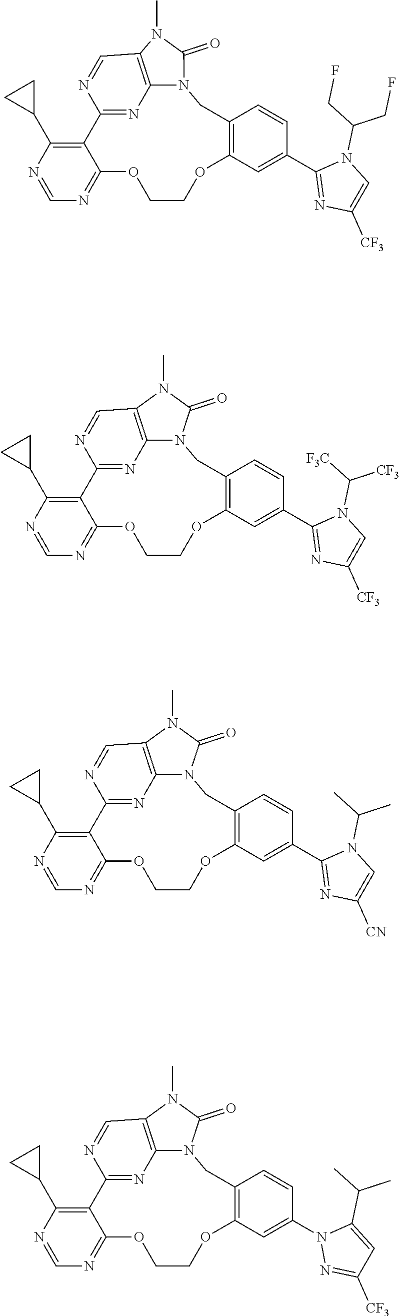 [SMILES: Cn1c(=O)n2c3nc(ncc31)-c1c(ncnc1C1CC1)OCCOc1cc(-c3nc(C(F)(F)F)cn3C(CF)CF)ccc1C2 ; Cn1c(=O)n2c3nc(ncc31)-c1c(ncnc1C1CC1)OCCOc1cc(-c3nc(C(F)(F)F)cn3C(C(F)(F)F)C(F)(F)F)ccc1C2 ; CC(C)n1cc(C#N)nc1-c1ccc2c(c1)OCCOc1ncnc(C3CC3)c1-c1ncc3c(n1)n(c(=O)n3C)C2 ; CC(C)c1cc(C(F)(F)F)nn1-c1ccc2c(c1)OCCOc1ncnc(C3CC3)c1-c1ncc3c(n1)n(c(=O)n3C)C2]

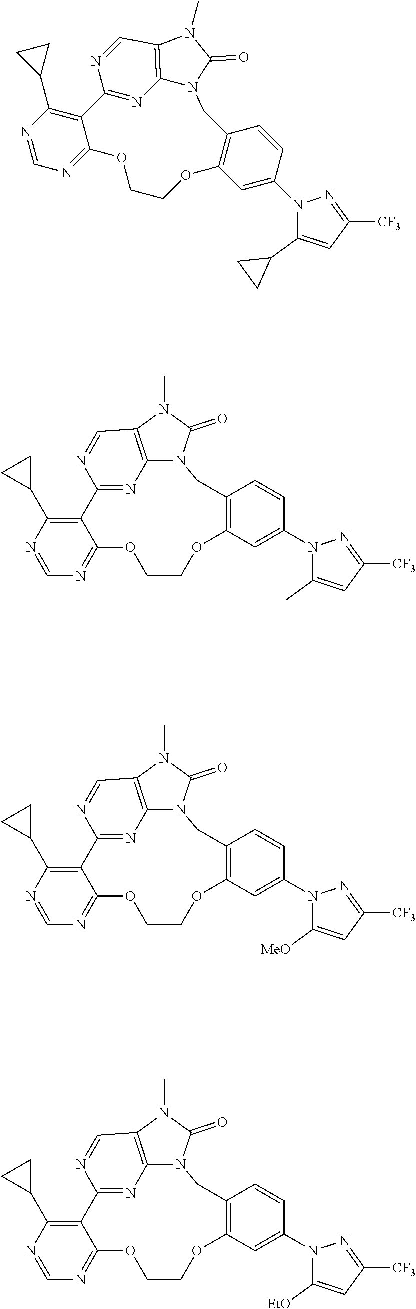 [SMILES: Cn1c(=O)n2c3nc(ncc31)-c1c(ncnc1C1CC1)OCCOc1cc(-n3nc(C(F)(F)F)cc3C3CC3)ccc1C2 ; Cc1cc(C(F)(F)F)nn1-c1ccc2c(c1)OCCOc1ncnc(C3CC3)c1-c1ncc3c(n1)n(c(=O)n3C)C2 ; COc1cc(C(F)(F)F)nn1-c1ccc2c(c1)OCCOc1ncnc(C3CC3)c1-c1ncc3c(n1)n(c(=O)n3C)C2 ; CCOc1cc(C(F)(F)F)nn1-c1ccc2c(c1)OCCOc1ncnc(C3CC3)c1-c1ncc3c(n1)n(c(=O)n3C)C2]

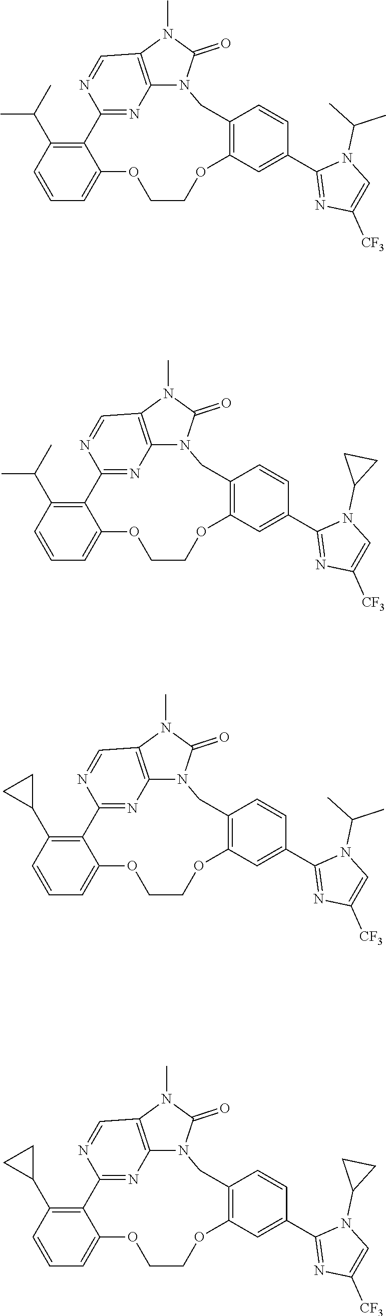

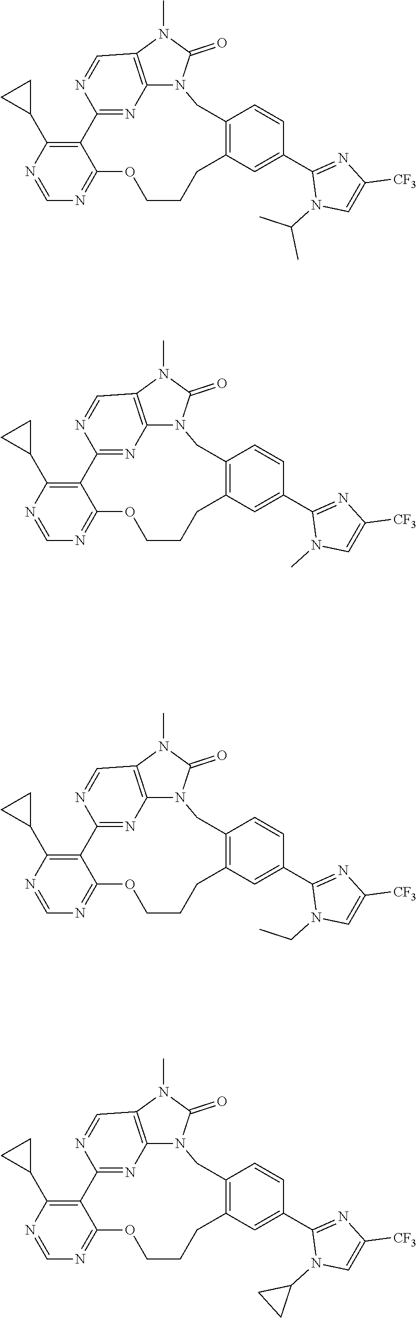 [SMILES: CC(C)n1cc(C(F)(F)F)nc1-c1ccc2c(c1)CCCOc1ncnc(C3CC3)c1-c1ncc3c(n1)n(c(=O)n3C)C2 ; Cn1cc(C(F)(F)F)nc1-c1ccc2c(c1)CCCOc1ncnc(C3CC3)c1-c1ncc3c(n1)n(c(=O)n3C)C2 ; CCn1cc(C(F)(F)F)nc1-c1ccc2c(c1)CCCOc1ncnc(C3CC3)c1-c1ncc3c(n1)n(c(=O)n3C)C2 ; Cn1c(=O)n2c3nc(ncc31)-c1c(ncnc1C1CC1)OCCCc1cc(-c3nc(C(F)(F)F)cn3C3CC3)ccc1C2]

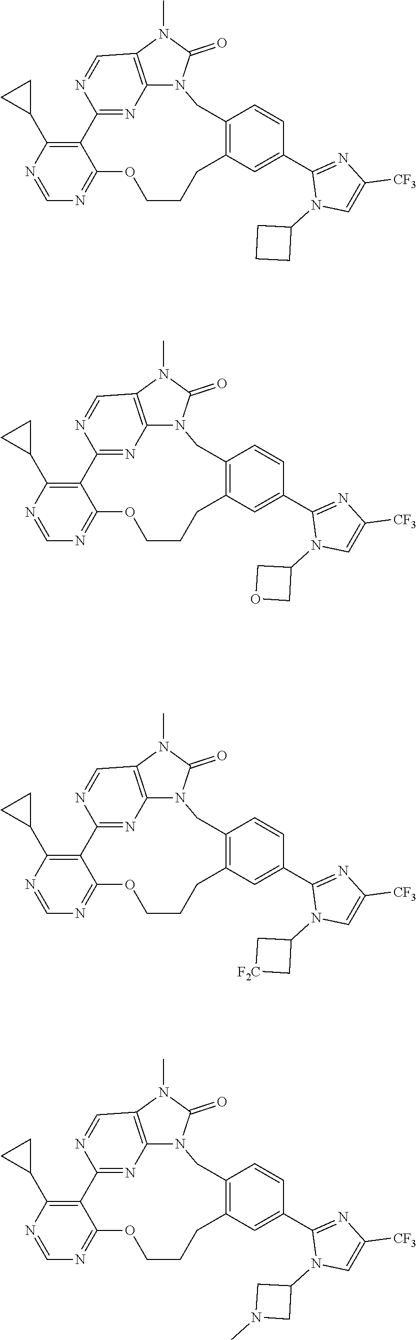 [SMILES: Cn1c(=O)n2c3nc(ncc31)-c1c(ncnc1C1CC1)OCCCc1cc(-c3nc(C(F)(F)F)cn3C3CCC3)ccc1C2 ; Cn1c(=O)n2c3nc(ncc31)-c1c(ncnc1C1CC1)OCCCc1cc(-c3nc(C(F)(F)F)cn3C3COC3)ccc1C2 ; Cn1c(=O)n2c3nc(ncc31)-c1c(ncnc1C1CC1)OCCCc1cc(-c3nc(C(F)(F)F)cn3C3CC(F)(F)C3)ccc1C2 ; CN1CC(n2cc(C(F)(F)F)nc2-c2ccc3c(c2)CCCOc2ncnc(C4CC4)c2-c2ncc4c(n2)n(c(=O)n4C)C3)C1]

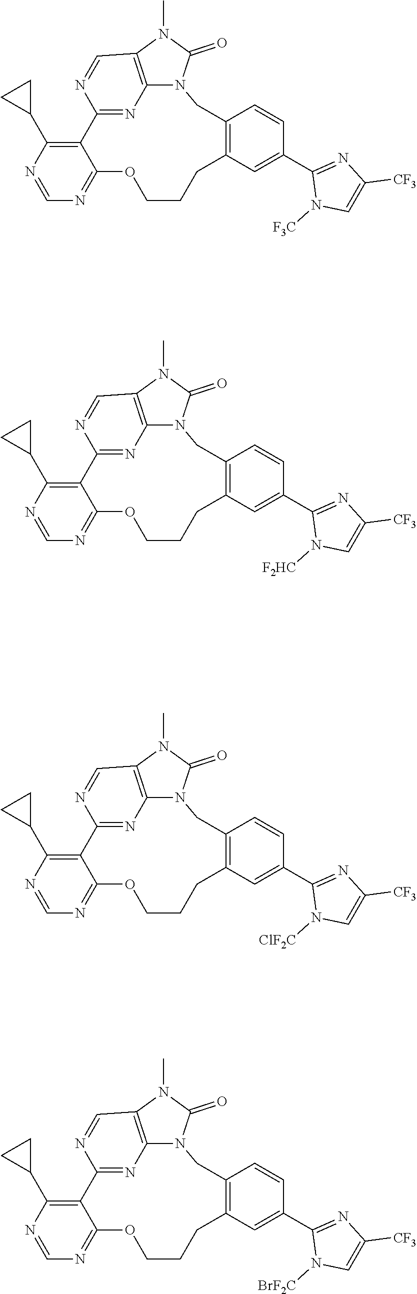

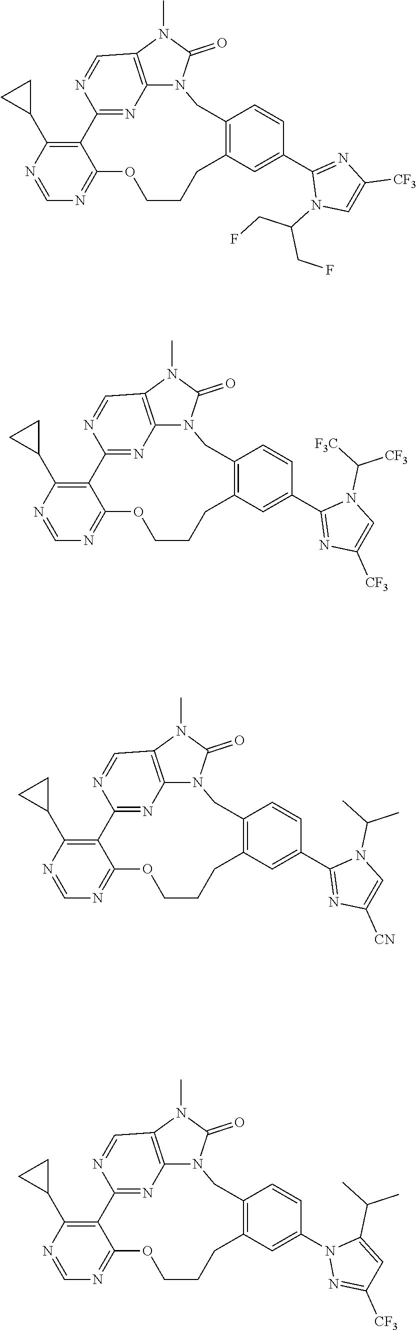 [SMILES: Cn1c(=O)n2c3nc(ncc31)-c1c(ncnc1C1CC1)OCCCc1cc(-c3nc(C(F)(F)F)cn3C(CF)CF)ccc1C2 ; Cn1c(=O)n2c3nc(ncc31)-c1c(ncnc1C1CC1)OCCCc1cc(-c3nc(C(F)(F)F)cn3C(C(F)(F)F)C(F)(F)F)ccc1C2 ; CC(C)n1cc(C#N)nc1-c1ccc2c(c1)CCCOc1ncnc(C3CC3)c1-c1ncc3c(n1)n(c(=O)n3C)C2 ; CC(C)c1cc(C(F)(F)F)nn1-c1ccc2c(c1)CCCOc1ncnc(C3CC3)c1-c1ncc3c(n1)n(c(=O)n3C)C2]

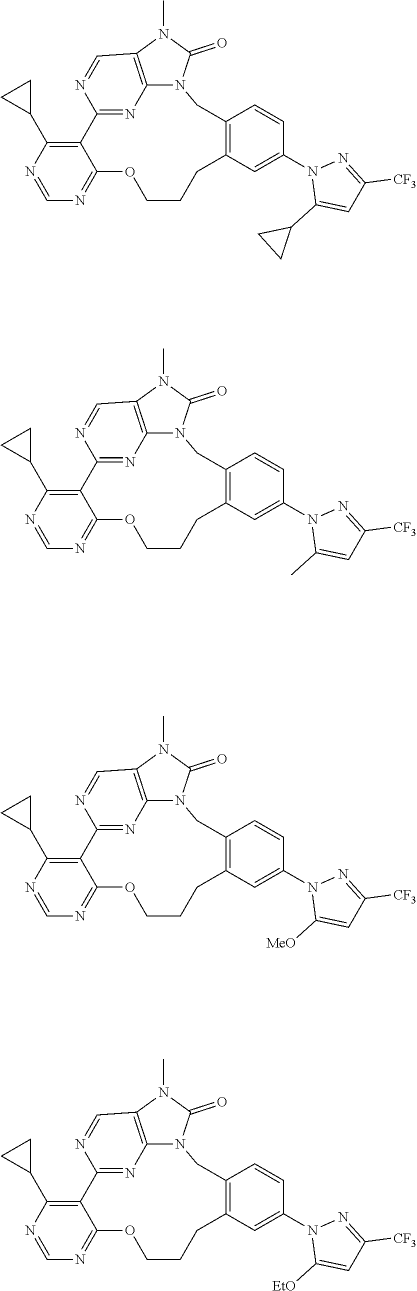 [SMILES: Cn1c(=O)n2c3nc(ncc31)-c1c(ncnc1C1CC1)OCCCc1cc(-n3nc(C(F)(F)F)cc3C3CC3)ccc1C2 ; Cc1cc(C(F)(F)F)nn1-c1ccc2c(c1)CCCOc1ncnc(C3CC3)c1-c1ncc3c(n1)n(c(=O)n3C)C2 ; COc1cc(C(F)(F)F)nn1-c1ccc2c(c1)CCCOc1ncnc(C3CC3)c1-c1ncc3c(n1)n(c(=O)n3C)C2 ; CCOc1cc(C(F)(F)F)nn1-c1ccc2c(c1)CCCOc1ncnc(C3CC3)c1-c1ncc3c(n1)n(c(=O)n3C)C2]

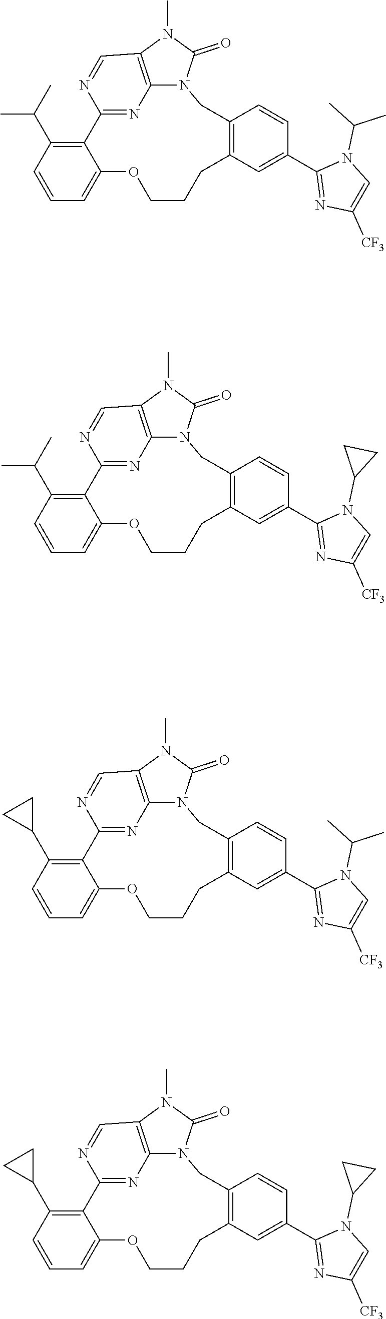

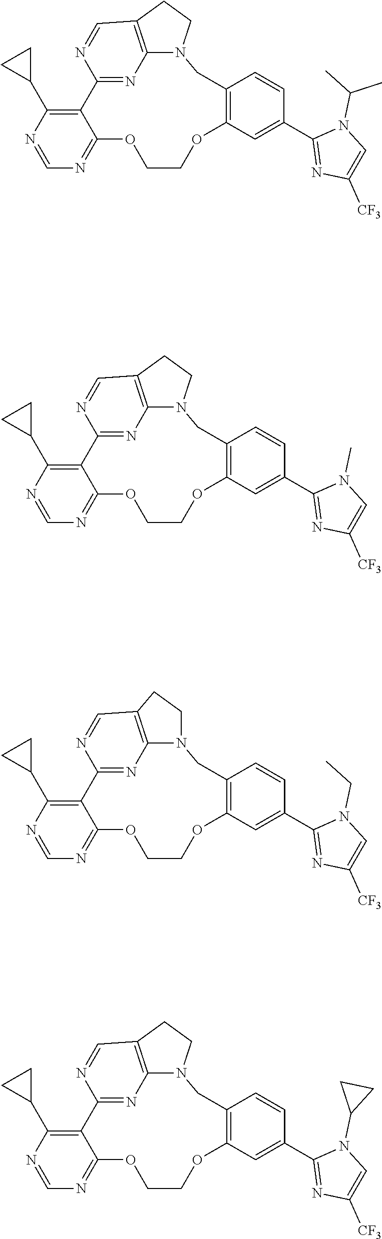 [SMILES: CC(C)n1cc(C(F)(F)F)nc1-c1ccc2c(c1)OCCOc1ncnc(C3CC3)c1-c1ncc3c(n1)N(CC3)C2 ; Cn1cc(C(F)(F)F)nc1-c1ccc2c(c1)OCCOc1ncnc(C3CC3)c1-c1ncc3c(n1)N(CC3)C2 ; CCn1cc(C(F)(F)F)nc1-c1ccc2c(c1)OCCOc1ncnc(C3CC3)c1-c1ncc3c(n1)N(CC3)C2 ; FC(F)(F)c1cn(C2CC2)c(-c2ccc3c(c2)OCCOc2ncnc(C4CC4)c2-c2ncc4c(n2)N(CC4)C3)n1]

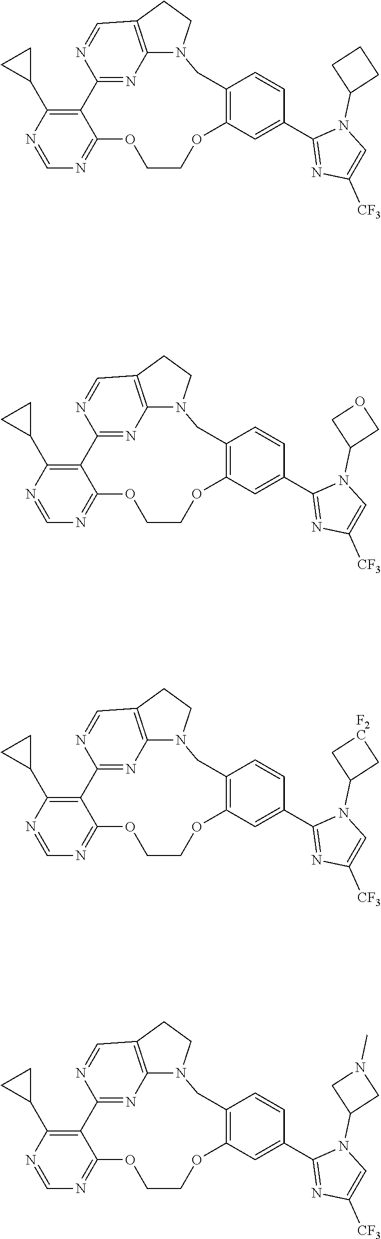 [SMILES: FC(F)(F)c1cn(C2CCC2)c(-c2ccc3c(c2)OCCOc2ncnc(C4CC4)c2-c2ncc4c(n2)N(CC4)C3)n1 ; FC(F)(F)c1cn(C2COC2)c(-c2ccc3c(c2)OCCOc2ncnc(C4CC4)c2-c2ncc4c(n2)N(CC4)C3)n1 ; FC1(F)CC(n2cc(C(F)(F)F)nc2-c2ccc3c(c2)OCCOc2ncnc(C4CC4)c2-c2ncc4c(n2)N(CC4)C3)C1 ; CN1CC(n2cc(C(F)(F)F)nc2-c2ccc3c(c2)OCCOc2ncnc(C4CC4)c2-c2ncc4c(n2)N(CC4)C3)C1]

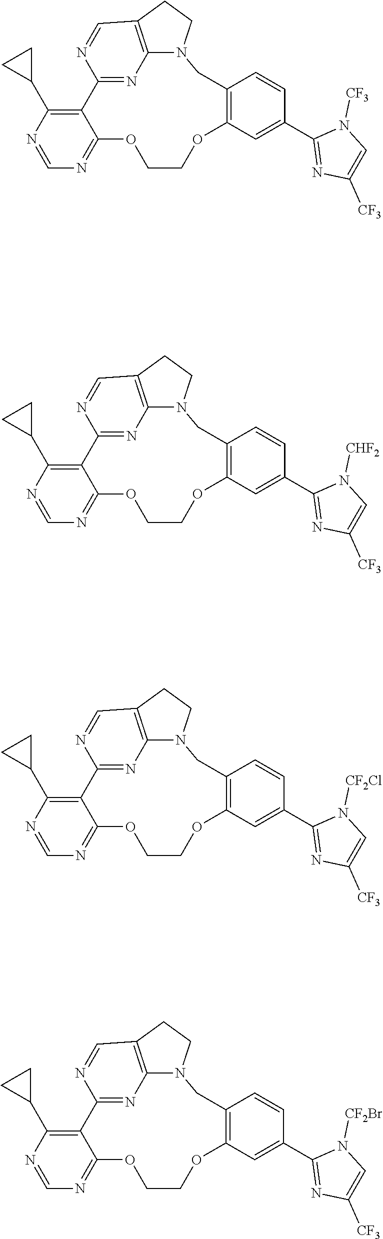

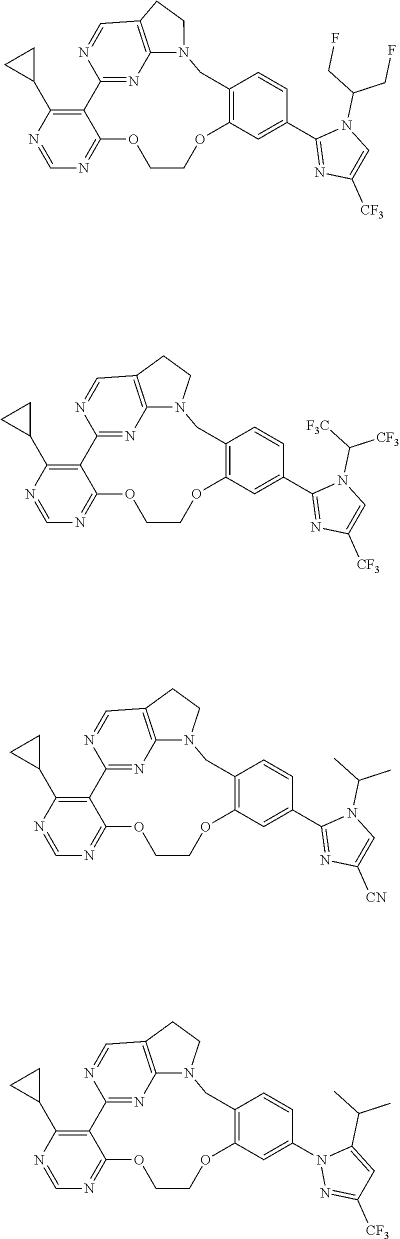 [SMILES: FCC(CF)n1cc(C(F)(F)F)nc1-c1ccc2c(c1)OCCOc1ncnc(C3CC3)c1-c1ncc3c(n1)N(CC3)C2 ; FC(F)(F)c1cn(C(C(F)(F)F)C(F)(F)F)c(-c2ccc3c(c2)OCCOc2ncnc(C4CC4)c2-c2ncc4c(n2)N(CC4)C3)n1 ; CC(C)n1cc(C#N)nc1-c1ccc2c(c1)OCCOc1ncnc(C3CC3)c1-c1ncc3c(n1)N(CC3)C2 ; CC(C)c1cc(C(F)(F)F)nn1-c1ccc2c(c1)OCCOc1ncnc(C3CC3)c1-c1ncc3c(n1)N(CC3)C2]

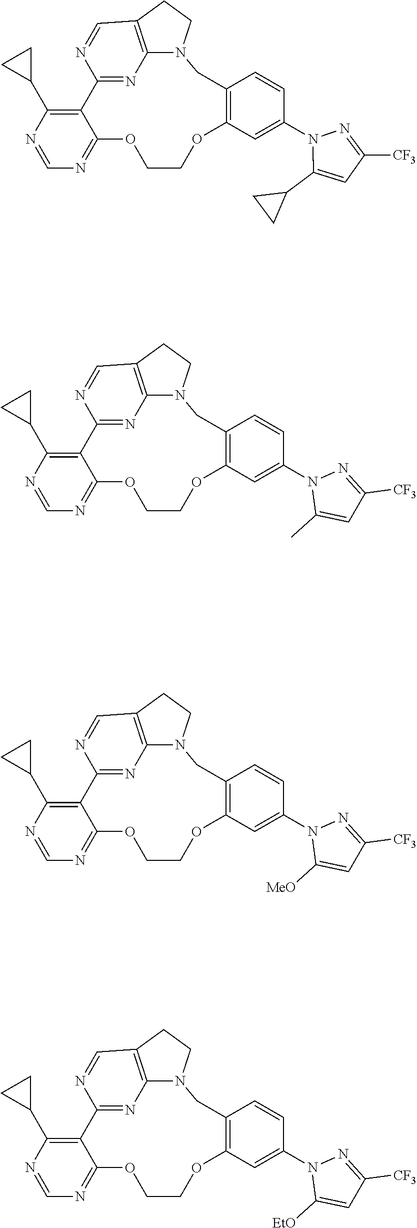 [SMILES: FC(F)(F)c1cc(C2CC2)n(-c2ccc3c(c2)OCCOc2ncnc(C4CC4)c2-c2ncc4c(n2)N(CC4)C3)n1 ; Cc1cc(C(F)(F)F)nn1-c1ccc2c(c1)OCCOc1ncnc(C3CC3)c1-c1ncc3c(n1)N(CC3)C2 ; COc1cc(C(F)(F)F)nn1-c1ccc2c(c1)OCCOc1ncnc(C3CC3)c1-c1ncc3c(n1)N(CC3)C2 ; CCOc1cc(C(F)(F)F)nn1-c1ccc2c(c1)OCCOc1ncnc(C3CC3)c1-c1ncc3c(n1)N(CC3)C2]

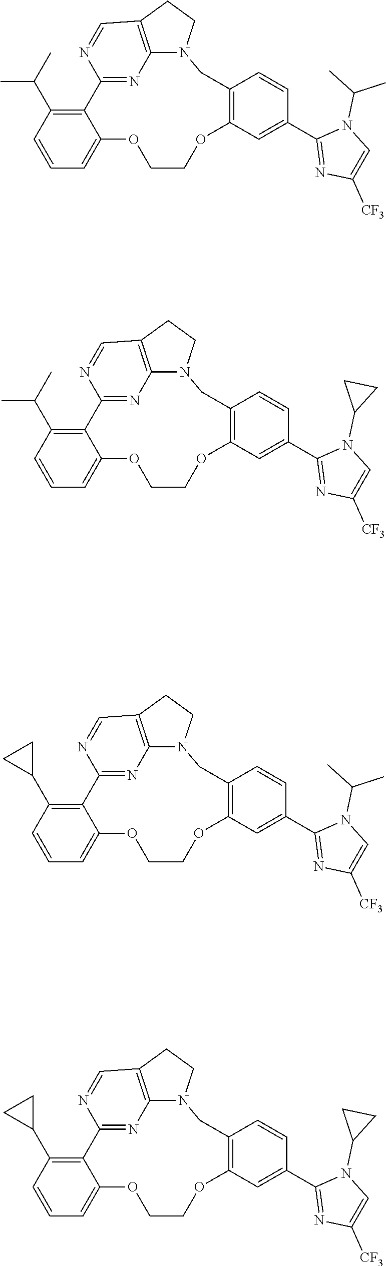

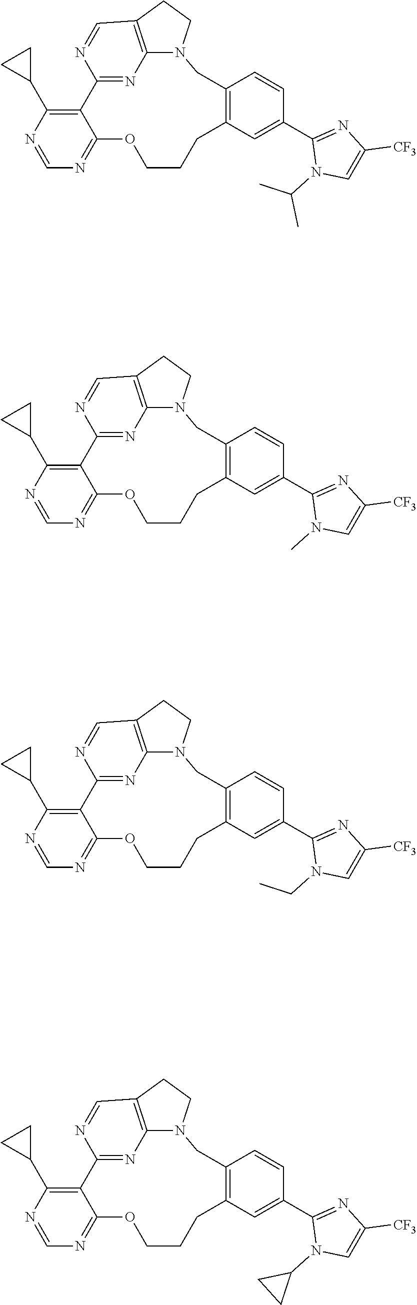 [SMILES: CC(C)n1cc(C(F)(F)F)nc1-c1ccc2c(c1)CCCOc1ncnc(C3CC3)c1-c1ncc3c(n1)N(CC3)C2 ; Cn1cc(C(F)(F)F)nc1-c1ccc2c(c1)CCCOc1ncnc(C3CC3)c1-c1ncc3c(n1)N(CC3)C2 ; CCn1cc(C(F)(F)F)nc1-c1ccc2c(c1)CCCOc1ncnc(C3CC3)c1-c1ncc3c(n1)N(CC3)C2 ; FC(F)(F)c1cn(C2CC2)c(-c2ccc3c(c2)CCCOc2ncnc(C4CC4)c2-c2ncc4c(n2)N(CC4)C3)n1]

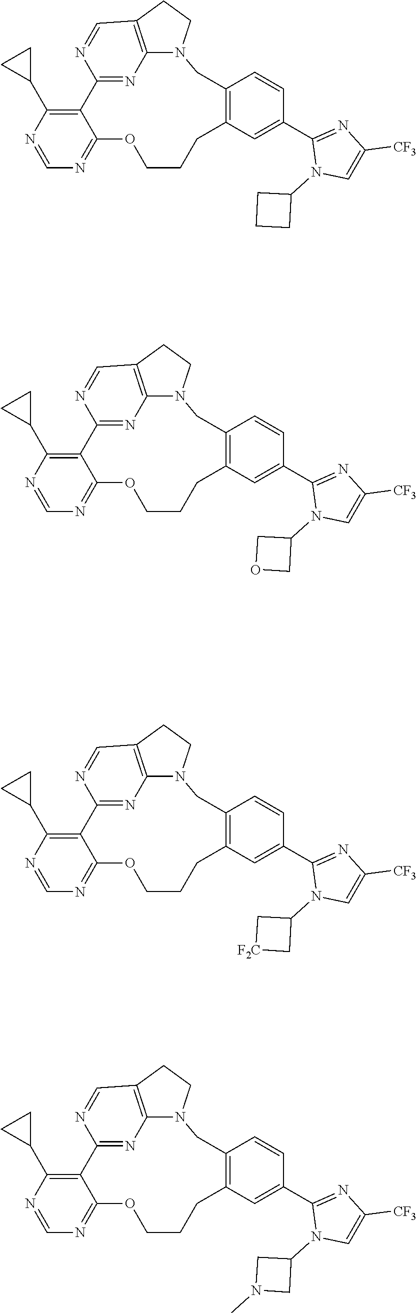 [SMILES: FC(F)(F)c1cn(C2CCC2)c(-c2ccc3c(c2)CCCOc2ncnc(C4CC4)c2-c2ncc4c(n2)N(CC4)C3)n1 ; FC(F)(F)c1cn(C2COC2)c(-c2ccc3c(c2)CCCOc2ncnc(C4CC4)c2-c2ncc4c(n2)N(CC4)C3)n1 ; FC1(F)CC(n2cc(C(F)(F)F)nc2-c2ccc3c(c2)CCCOc2ncnc(C4CC4)c2-c2ncc4c(n2)N(CC4)C3)C1 ; CN1CC(n2cc(C(F)(F)F)nc2-c2ccc3c(c2)CCCOc2ncnc(C4CC4)c2-c2ncc4c(n2)N(CC4)C3)C1]

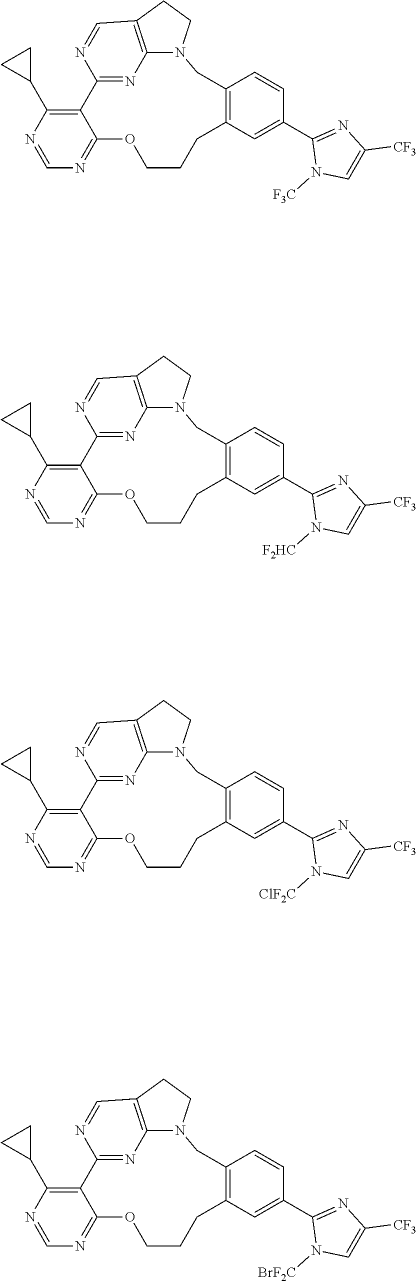

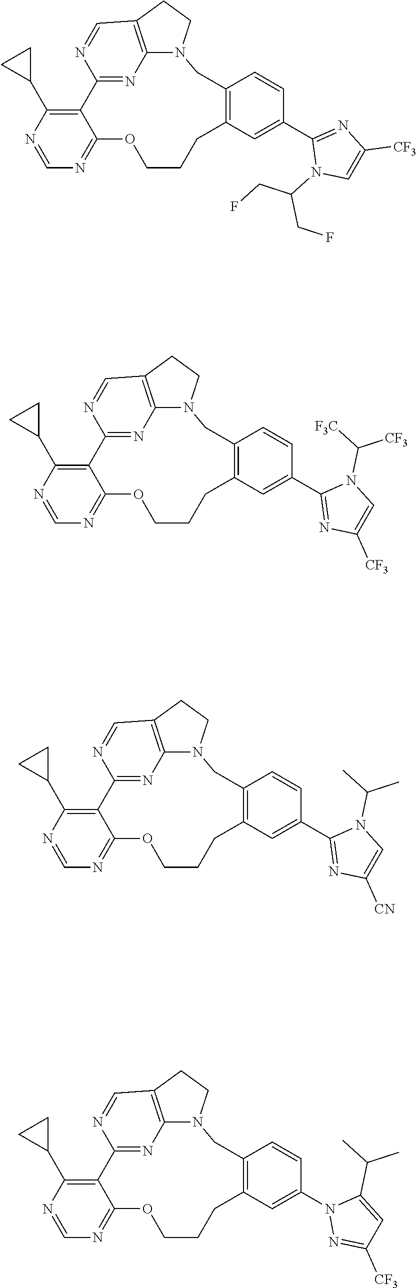 [SMILES: FCC(CF)n1cc(C(F)(F)F)nc1-c1ccc2c(c1)CCCOc1ncnc(C3CC3)c1-c1ncc3c(n1)N(CC3)C2 ; FC(F)(F)c1cn(C(C(F)(F)F)C(F)(F)F)c(-c2ccc3c(c2)CCCOc2ncnc(C4CC4)c2-c2ncc4c(n2)N(CC4)C3)n1 ; CC(C)n1cc(C#N)nc1-c1ccc2c(c1)CCCOc1ncnc(C3CC3)c1-c1ncc3c(n1)N(CC3)C2 ; CC(C)c1cc(C(F)(F)F)nn1-c1ccc2c(c1)CCCOc1ncnc(C3CC3)c1-c1ncc3c(n1)N(CC3)C2]

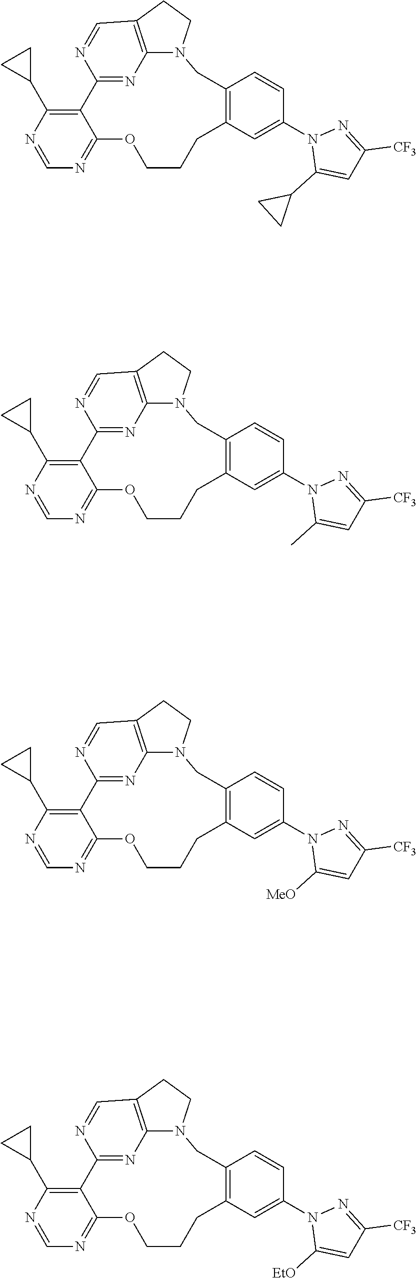 [SMILES: FC(F)(F)c1cc(C2CC2)n(-c2ccc3c(c2)CCCOc2ncnc(C4CC4)c2-c2ncc4c(n2)N(CC4)C3)n1 ; Cc1cc(C(F)(F)F)nn1-c1ccc2c(c1)CCCOc1ncnc(C3CC3)c1-c1ncc3c(n1)N(CC3)C2 ; COc1cc(C(F)(F)F)nn1-c1ccc2c(c1)CCCOc1ncnc(C3CC3)c1-c1ncc3c(n1)N(CC3)C2 ; CCOc1cc(C(F)(F)F)nn1-c1ccc2c(c1)CCCOc1ncnc(C3CC3)c1-c1ncc3c(n1)N(CC3)C2]

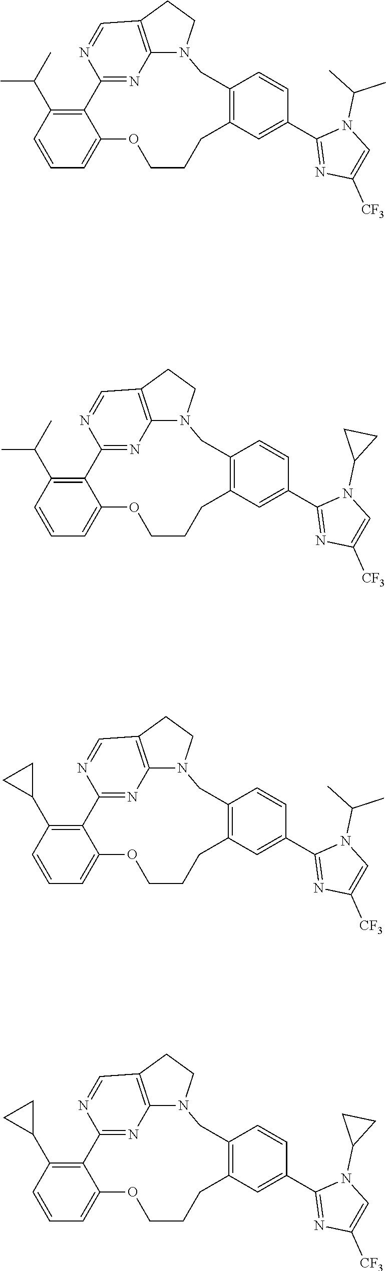

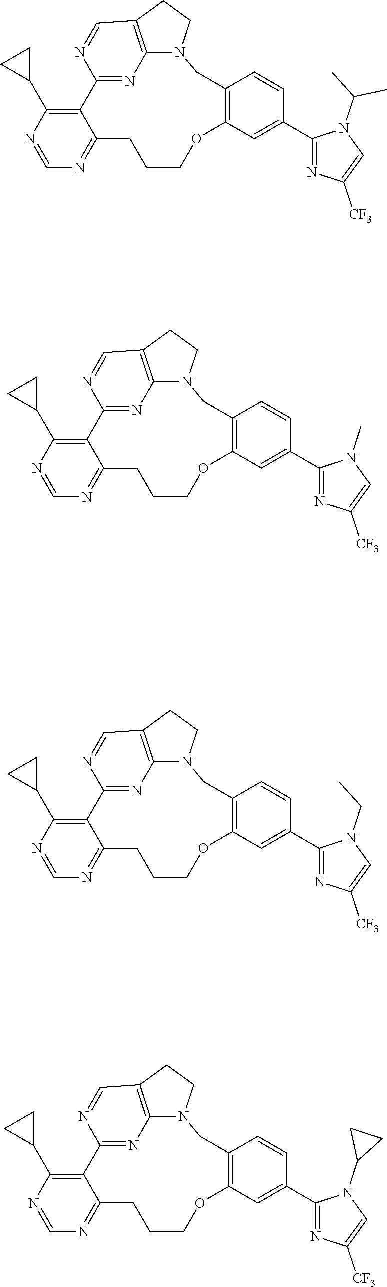 [SMILES: CC(C)n1cc(C(F)(F)F)nc1-c1ccc2c(c1)OCCCc1ncnc(C3CC3)c1-c1ncc3c(n1)N(CC3)C2 ; Cn1cc(C(F)(F)F)nc1-c1ccc2c(c1)OCCCc1ncnc(C3CC3)c1-c1ncc3c(n1)N(CC3)C2 ; CCn1cc(C(F)(F)F)nc1-c1ccc2c(c1)OCCCc1ncnc(C3CC3)c1-c1ncc3c(n1)N(CC3)C2 ; FC(F)(F)c1cn(C2CC2)c(-c2ccc3c(c2)OCCCc2ncnc(C4CC4)c2-c2ncc4c(n2)N(CC4)C3)n1]

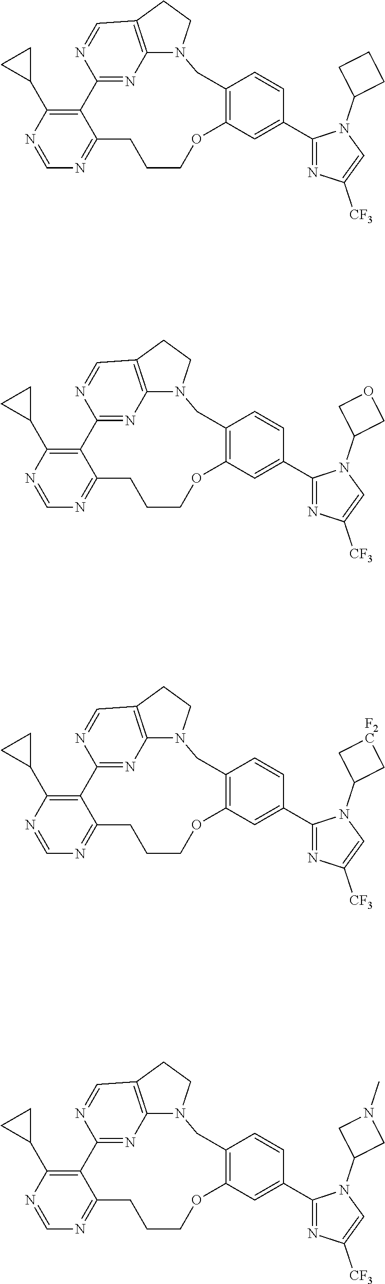 [SMILES: FC(F)(F)c1cn(C2CCC2)c(-c2ccc3c(c2)OCCCc2ncnc(C4CC4)c2-c2ncc4c(n2)N(CC4)C3)n1 ; FC(F)(F)c1cn(C2COC2)c(-c2ccc3c(c2)OCCCc2ncnc(C4CC4)c2-c2ncc4c(n2)N(CC4)C3)n1 ; FC1(F)CC(n2cc(C(F)(F)F)nc2-c2ccc3c(c2)OCCCc2ncnc(C4CC4)c2-c2ncc4c(n2)N(CC4)C3)C1 ; CN1CC(n2cc(C(F)(F)F)nc2-c2ccc3c(c2)OCCCc2ncnc(C4CC4)c2-c2ncc4c(n2)N(CC4)C3)C1]

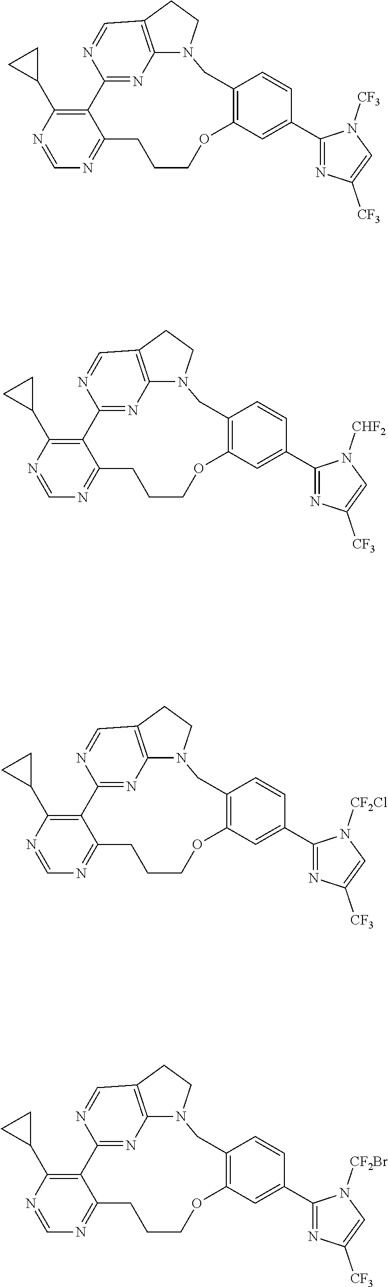

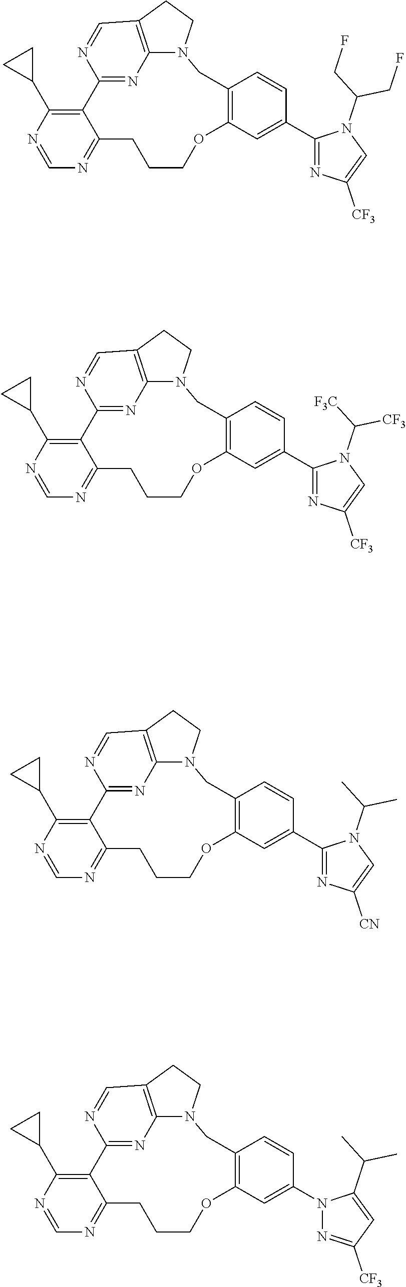 [SMILES: FCC(CF)n1cc(C(F)(F)F)nc1-c1ccc2c(c1)OCCCc1ncnc(C3CC3)c1-c1ncc3c(n1)N(CC3)C2 ; FC(F)(F)c1cn(C(C(F)(F)F)C(F)(F)F)c(-c2ccc3c(c2)OCCCc2ncnc(C4CC4)c2-c2ncc4c(n2)N(CC4)C3)n1 ; CC(C)n1cc(C#N)nc1-c1ccc2c(c1)OCCCc1ncnc(C3CC3)c1-c1ncc3c(n1)N(CC3)C2 ; CC(C)c1cc(C(F)(F)F)nn1-c1ccc2c(c1)OCCCc1ncnc(C3CC3)c1-c1ncc3c(n1)N(CC3)C2]

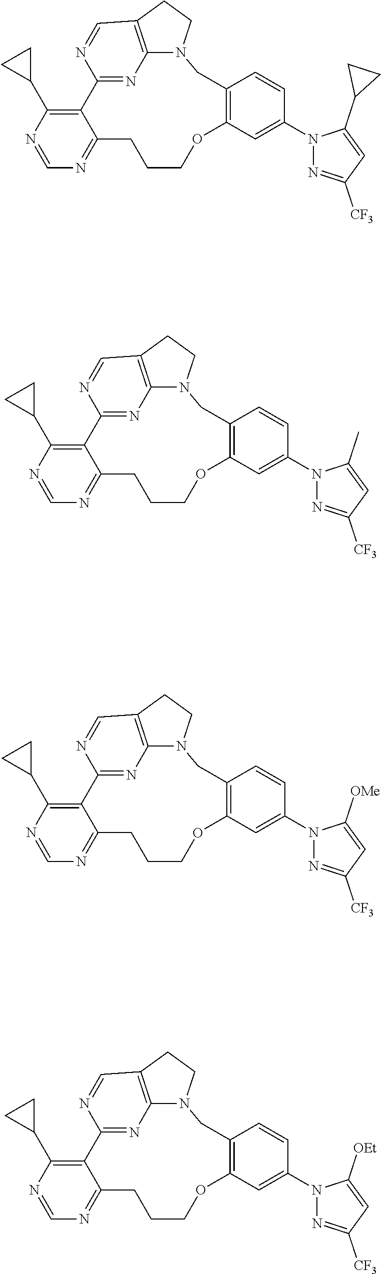 [SMILES: FC(F)(F)c1cc(C2CC2)n(-c2ccc3c(c2)OCCCc2ncnc(C4CC4)c2-c2ncc4c(n2)N(CC4)C3)n1 ; Cc1cc(C(F)(F)F)nn1-c1ccc2c(c1)OCCCc1ncnc(C3CC3)c1-c1ncc3c(n1)N(CC3)C2 ; COc1cc(C(F)(F)F)nn1-c1ccc2c(c1)OCCCc1ncnc(C3CC3)c1-c1ncc3c(n1)N(CC3)C2 ; CCOc1cc(C(F)(F)F)nn1-c1ccc2c(c1)OCCCc1ncnc(C3CC3)c1-c1ncc3c(n1)N(CC3)C2]

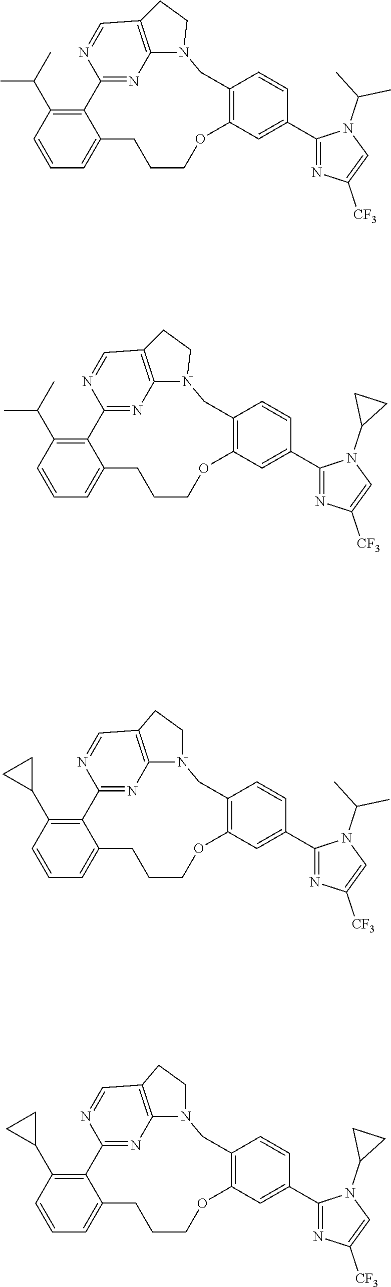

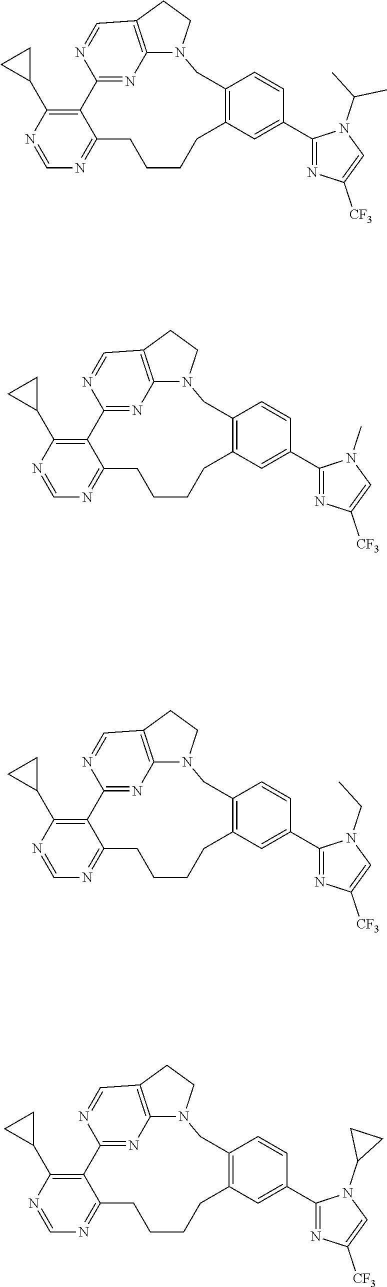 [SMILES: CC(C)n1cc(C(F)(F)F)nc1-c1ccc2c(c1)CCCCc1ncnc(C3CC3)c1-c1ncc3c(n1)N(CC3)C2 ; Cn1cc(C(F)(F)F)nc1-c1ccc2c(c1)CCCCc1ncnc(C3CC3)c1-c1ncc3c(n1)N(CC3)C2 ; CCn1cc(C(F)(F)F)nc1-c1ccc2c(c1)CCCCc1ncnc(C3CC3)c1-c1ncc3c(n1)N(CC3)C2 ; FC(F)(F)c1cn(C2CC2)c(-c2ccc3c(c2)CCCCc2ncnc(C4CC4)c2-c2ncc4c(n2)N(CC4)C3)n1]

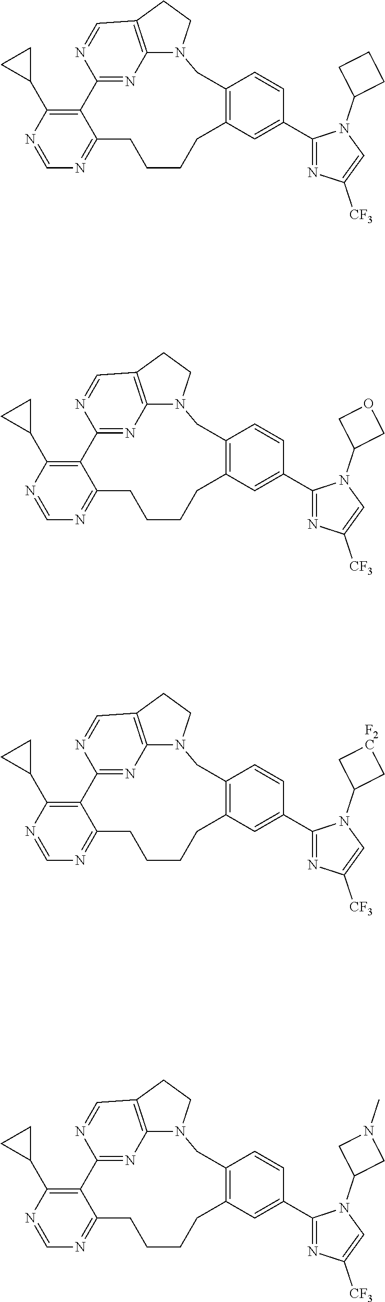 [SMILES: FC(F)(F)c1cn(C2CCC2)c(-c2ccc3c(c2)CCCCc2ncnc(C4CC4)c2-c2ncc4c(n2)N(CC4)C3)n1 ; FC(F)(F)c1cn(C2COC2)c(-c2ccc3c(c2)CCCCc2ncnc(C4CC4)c2-c2ncc4c(n2)N(CC4)C3)n1 ; FC1(F)CC(n2cc(C(F)(F)F)nc2-c2ccc3c(c2)CCCCc2ncnc(C4CC4)c2-c2ncc4c(n2)N(CC4)C3)C1 ; CN1CC(n2cc(C(F)(F)F)nc2-c2ccc3c(c2)CCCCc2ncnc(C4CC4)c2-c2ncc4c(n2)N(CC4)C3)C1]

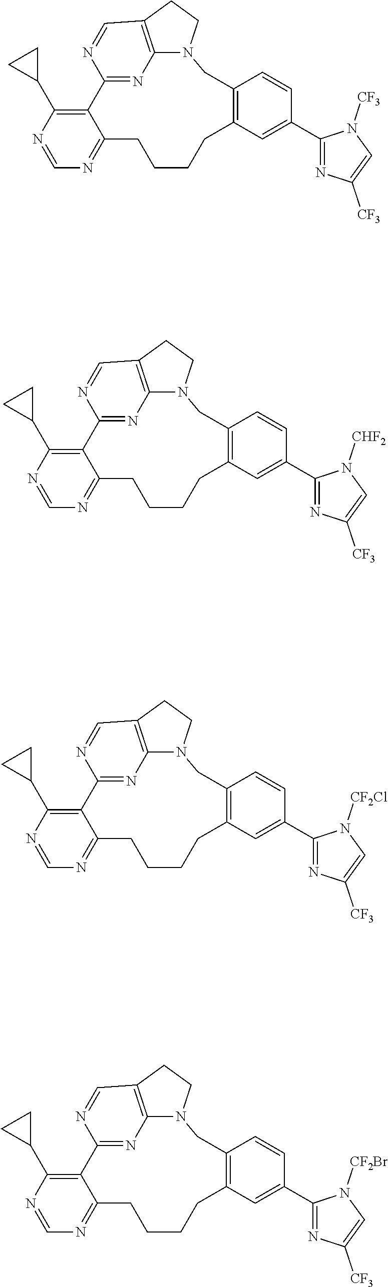

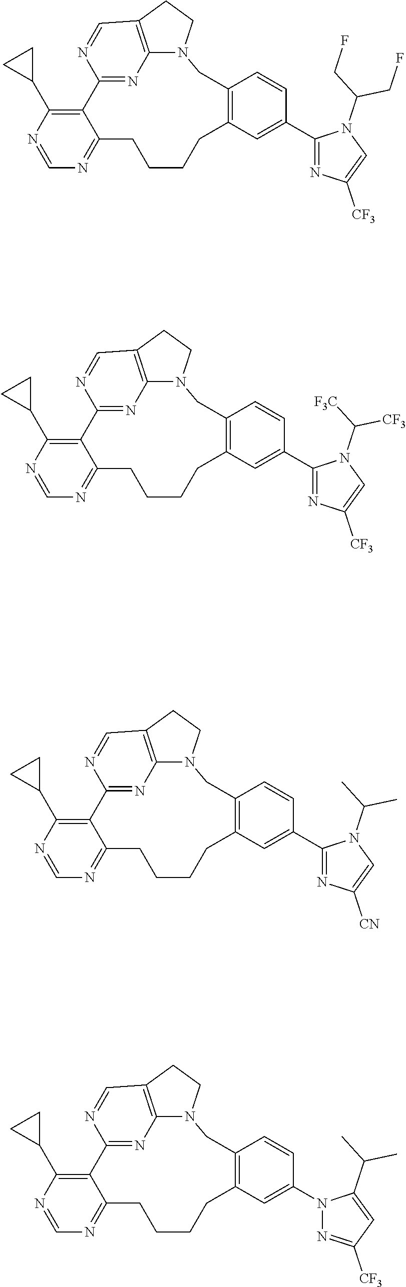 [SMILES: FCC(CF)n1cc(C(F)(F)F)nc1-c1ccc2c(c1)CCCCc1ncnc(C3CC3)c1-c1ncc3c(n1)N(CC3)C2 ; FC(F)(F)c1cn(C(C(F)(F)F)C(F)(F)F)c(-c2ccc3c(c2)CCCCc2ncnc(C4CC4)c2-c2ncc4c(n2)N(CC4)C3)n1 ; CC(C)n1cc(C#N)nc1-c1ccc2c(c1)CCCCc1ncnc(C3CC3)c1-c1ncc3c(n1)N(CC3)C2 ; CC(C)c1cc(C(F)(F)F)nn1-c1ccc2c(c1)CCCCc1ncnc(C3CC3)c1-c1ncc3c(n1)N(CC3)C2]

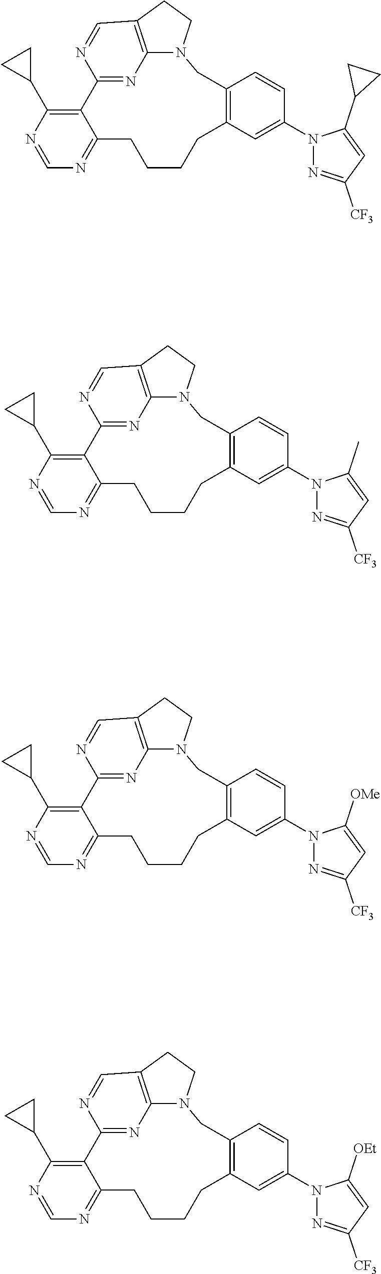 [SMILES: FC(F)(F)c1cc(C2CC2)n(-c2ccc3c(c2)CCCCc2ncnc(C4CC4)c2-c2ncc4c(n2)N(CC4)C3)n1 ; Cc1cc(C(F)(F)F)nn1-c1ccc2c(c1)CCCCc1ncnc(C3CC3)c1-c1ncc3c(n1)N(CC3)C2 ; COc1cc(C(F)(F)F)nn1-c1ccc2c(c1)CCCCc1ncnc(C3CC3)c1-c1ncc3c(n1)N(CC3)C2 ; CCOc1cc(C(F)(F)F)nn1-c1ccc2c(c1)CCCCc1ncnc(C3CC3)c1-c1ncc3c(n1)N(CC3)C2]

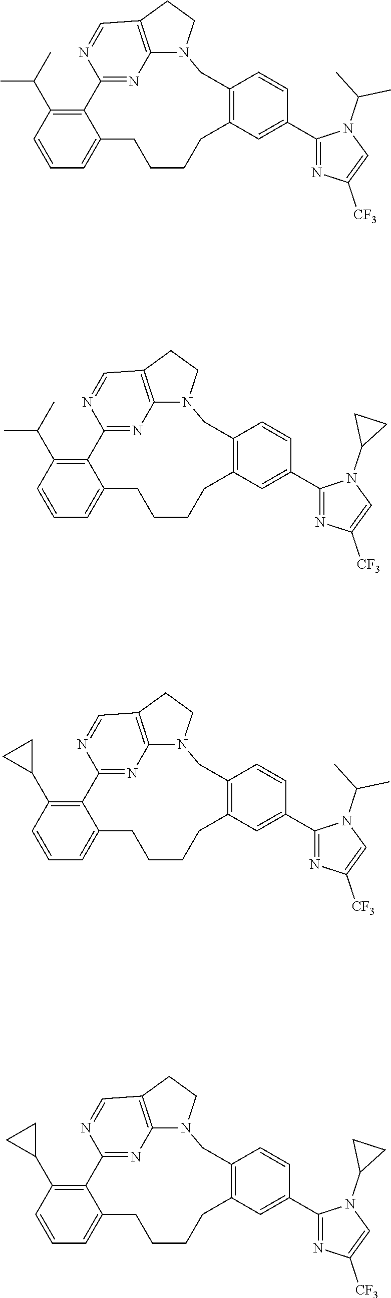

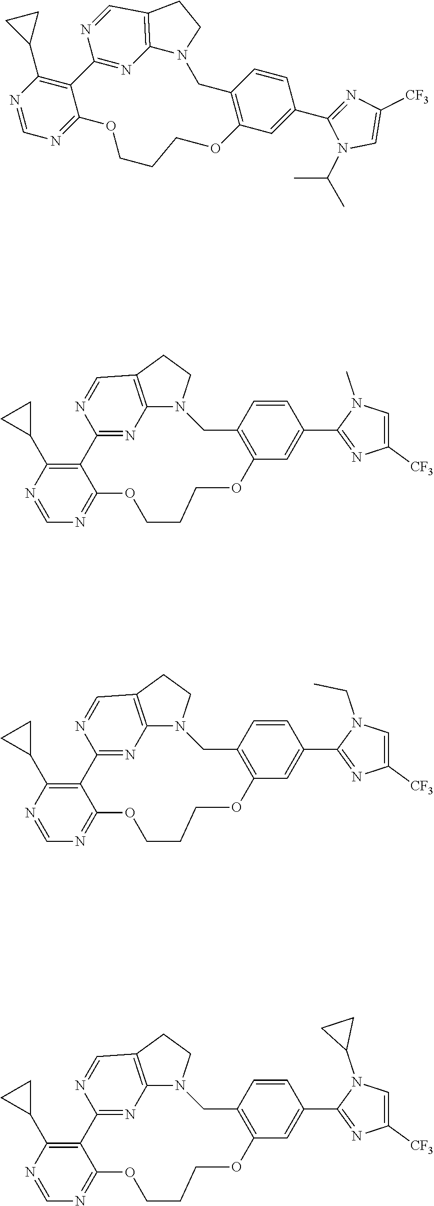 [SMILES: CC(C)n1cc(C(F)(F)F)nc1-c1ccc2c(c1)OCCCOc1ncnc(C3CC3)c1-c1ncc3c(n1)N(CC3)C2 ; Cn1cc(C(F)(F)F)nc1-c1ccc2c(c1)OCCCOc1ncnc(C3CC3)c1-c1ncc3c(n1)N(CC3)C2 ; CCn1cc(C(F)(F)F)nc1-c1ccc2c(c1)OCCCOc1ncnc(C3CC3)c1-c1ncc3c(n1)N(CC3)C2 ; FC(F)(F)c1cn(C2CC2)c(-c2ccc3c(c2)OCCCOc2ncnc(C4CC4)c2-c2ncc4c(n2)N(CC4)C3)n1]

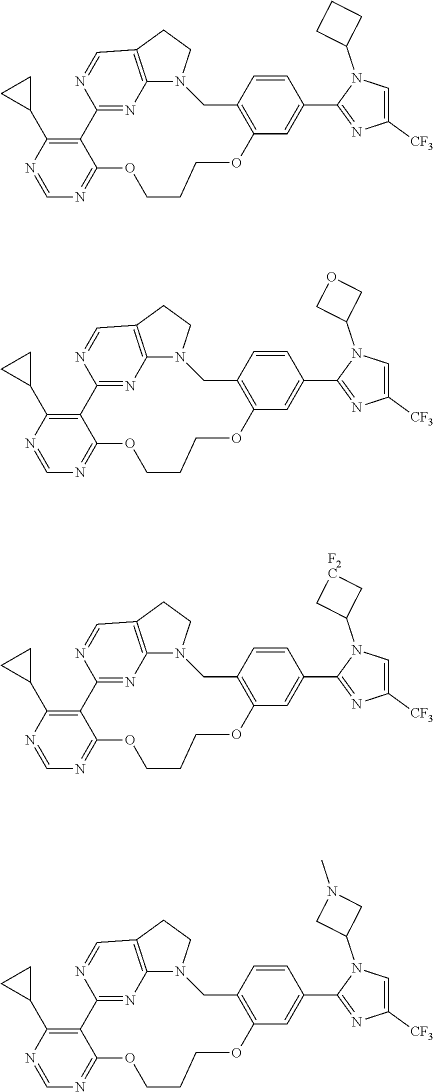 [SMILES: FC(F)(F)c1cn(C2CCC2)c(-c2ccc3c(c2)OCCCOc2ncnc(C4CC4)c2-c2ncc4c(n2)N(CC4)C3)n1 ; FC(F)(F)c1cn(C2COC2)c(-c2ccc3c(c2)OCCCOc2ncnc(C4CC4)c2-c2ncc4c(n2)N(CC4)C3)n1 ; FC1(F)CC(n2cc(C(F)(F)F)nc2-c2ccc3c(c2)OCCCOc2ncnc(C4CC4)c2-c2ncc4c(n2)N(CC4)C3)C1 ; CN1CC(n2cc(C(F)(F)F)nc2-c2ccc3c(c2)OCCCOc2ncnc(C4CC4)c2-c2ncc4c(n2)N(CC4)C3)C1]

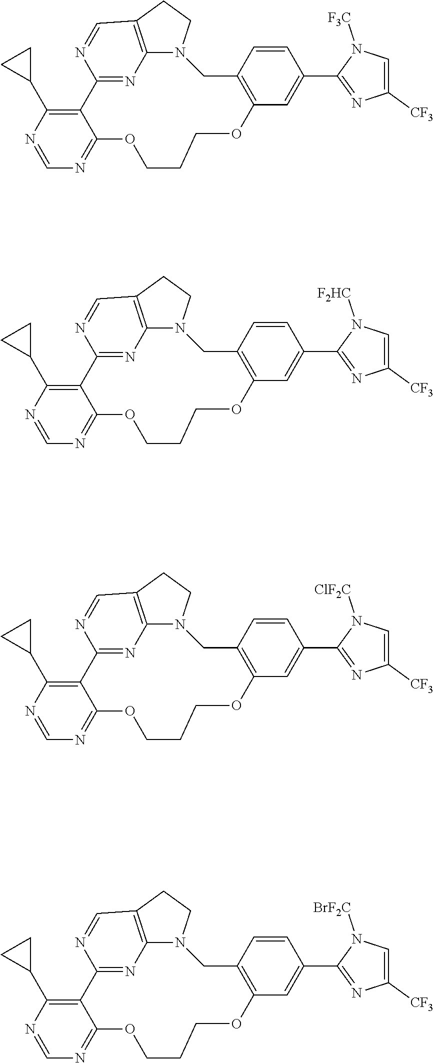

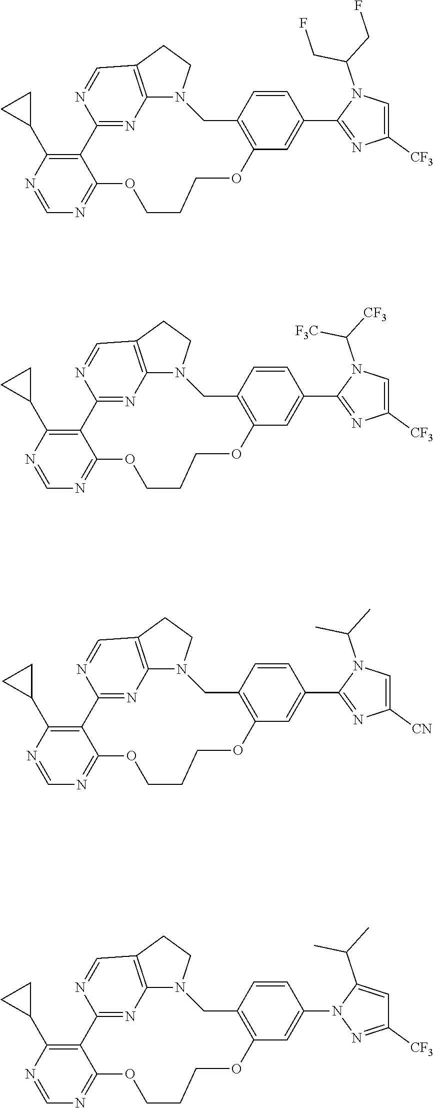 [SMILES: FCC(CF)n1cc(C(F)(F)F)nc1-c1ccc2c(c1)OCCCOc1ncnc(C3CC3)c1-c1ncc3c(n1)N(CC3)C2 ; FC(F)(F)c1cn(C(C(F)(F)F)C(F)(F)F)c(-c2ccc3c(c2)OCCCOc2ncnc(C4CC4)c2-c2ncc4c(n2)N(CC4)C3)n1 ; CC(C)n1cc(C#N)nc1-c1ccc2c(c1)OCCCOc1ncnc(C3CC3)c1-c1ncc3c(n1)N(CC3)C2 ; CC(C)c1cc(C(F)(F)F)nn1-c1ccc2c(c1)OCCCOc1ncnc(C3CC3)c1-c1ncc3c(n1)N(CC3)C2]

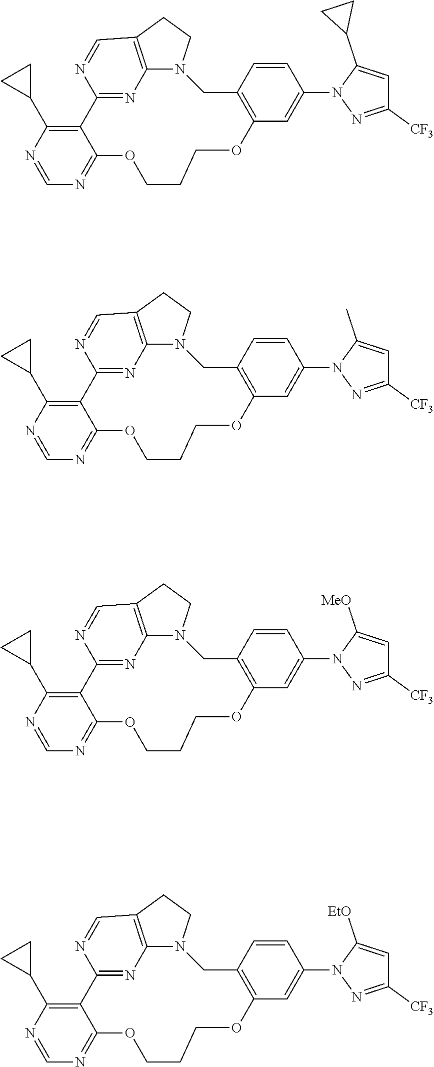 [SMILES: FC(F)(F)c1cc(C2CC2)n(-c2ccc3c(c2)OCCCOc2ncnc(C4CC4)c2-c2ncc4c(n2)N(CC4)C3)n1 ; Cc1cc(C(F)(F)F)nn1-c1ccc2c(c1)OCCCOc1ncnc(C3CC3)c1-c1ncc3c(n1)N(CC3)C2 ; COc1cc(C(F)(F)F)nn1-c1ccc2c(c1)OCCCOc1ncnc(C3CC3)c1-c1ncc3c(n1)N(CC3)C2 ; CCOc1cc(C(F)(F)F)nn1-c1ccc2c(c1)OCCCOc1ncnc(C3CC3)c1-c1ncc3c(n1)N(CC3)C2]

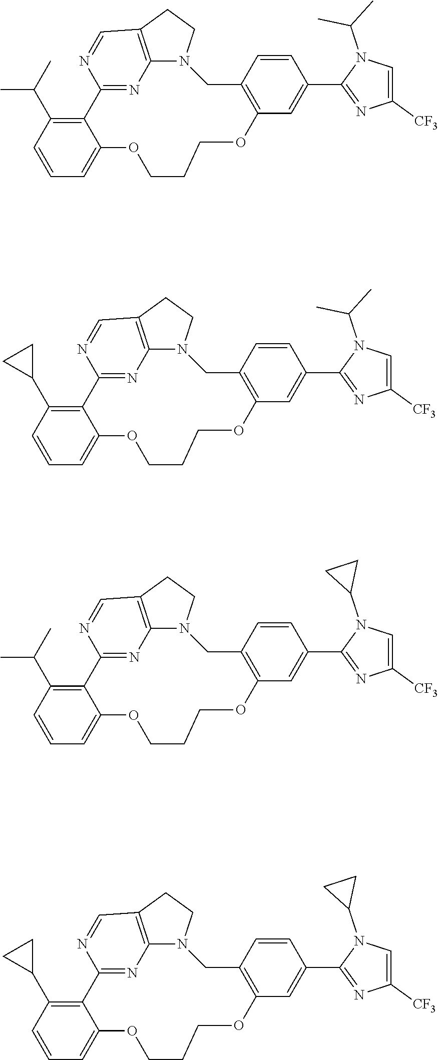 [SMILES: CC(C)c1cccc2c1-c1ncc3c(n1)N(CC3)Cc1ccc(-c3nc(C(F)(F)F)cn3C(C)C)cc1OCCCO2 ; CC(C)n1cc(C(F)(F)F)nc1-c1ccc2c(c1)OCCCOc1cccc(C3CC3)c1-c1ncc3c(n1)N(CC3)C2 ; CC(C)c1cccc2c1-c1ncc3c(n1)N(CC3)Cc1ccc(-c3nc(C(F)(F)F)cn3C3CC3)cc1OCCCO2 ; FC(F)(F)c1cn(C2CC2)c(-c2ccc3c(c2)OCCCOc2cccc(C4CC4)c2-c2ncc4c(n2)N(CC4)C3)n1]

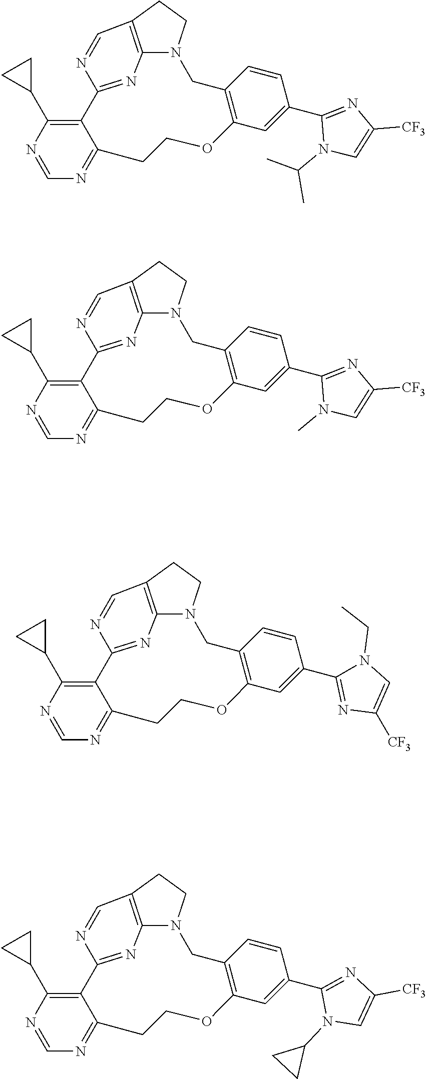 [SMILES: CC(C)n1cc(C(F)(F)F)nc1-c1ccc2c(c1)OCCc1ncnc(C3CC3)c1-c1ncc3c(n1)N(CC3)C2 ; Cn1cc(C(F)(F)F)nc1-c1ccc2c(c1)OCCc1ncnc(C3CC3)c1-c1ncc3c(n1)N(CC3)C2 ; CCn1cc(C(F)(F)F)nc1-c1ccc2c(c1)OCCc1ncnc(C3CC3)c1-c1ncc3c(n1)N(CC3)C2 ; FC(F)(F)c1cn(C2CC2)c(-c2ccc3c(c2)OCCc2ncnc(C4CC4)c2-c2ncc4c(n2)N(CC4)C3)n1]

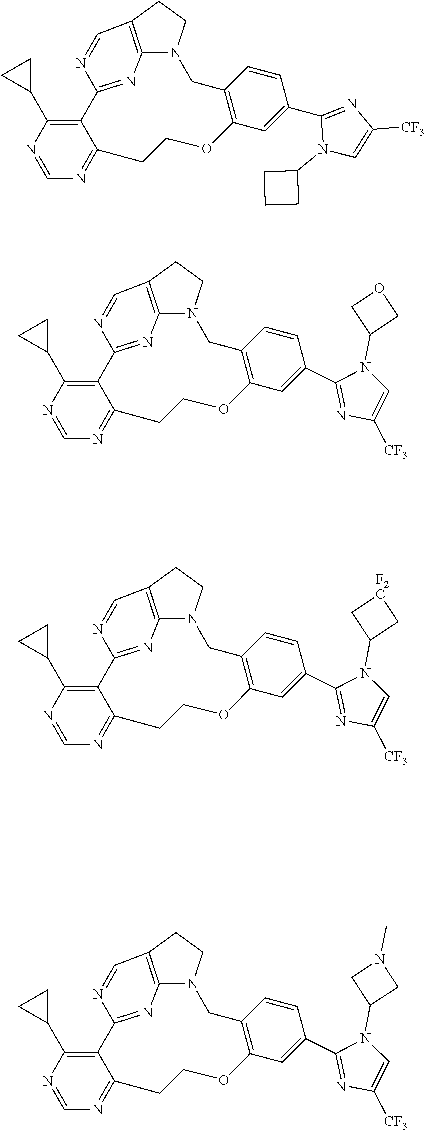 [SMILES: FC(F)(F)c1cn(C2CCC2)c(-c2ccc3c(c2)OCCc2ncnc(C4CC4)c2-c2ncc4c(n2)N(CC4)C3)n1 ; FC(F)(F)c1cn(C2COC2)c(-c2ccc3c(c2)OCCc2ncnc(C4CC4)c2-c2ncc4c(n2)N(CC4)C3)n1 ; FC1(F)CC(n2cc(C(F)(F)F)nc2-c2ccc3c(c2)OCCc2ncnc(C4CC4)c2-c2ncc4c(n2)N(CC4)C3)C1 ; CN1CC(n2cc(C(F)(F)F)nc2-c2ccc3c(c2)OCCc2ncnc(C4CC4)c2-c2ncc4c(n2)N(CC4)C3)C1]

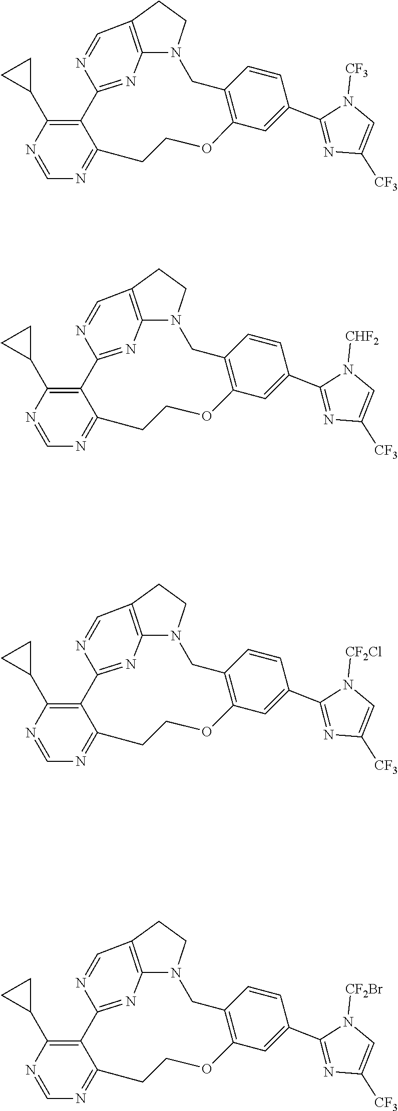

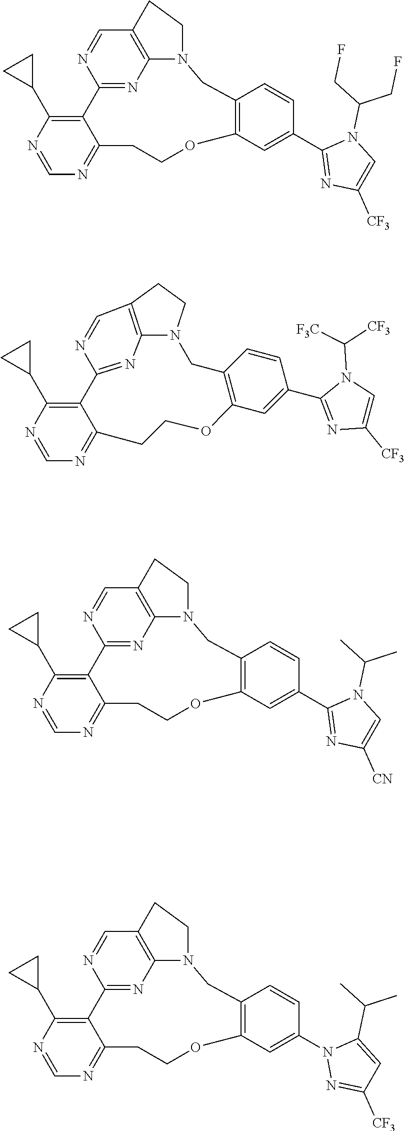 [SMILES: FCC(CF)n1cc(C(F)(F)F)nc1-c1ccc2c(c1)OCCc1ncnc(C3CC3)c1-c1ncc3c(n1)N(CC3)C2 ; FC(F)(F)c1cn(C(C(F)(F)F)C(F)(F)F)c(-c2ccc3c(c2)OCCc2ncnc(C4CC4)c2-c2ncc4c(n2)N(CC4)C3)n1 ; CC(C)n1cc(C#N)nc1-c1ccc2c(c1)OCCc1ncnc(C3CC3)c1-c1ncc3c(n1)N(CC3)C2 ; CC(C)c1cc(C(F)(F)F)nn1-c1ccc2c(c1)OCCc1ncnc(C3CC3)c1-c1ncc3c(n1)N(CC3)C2]

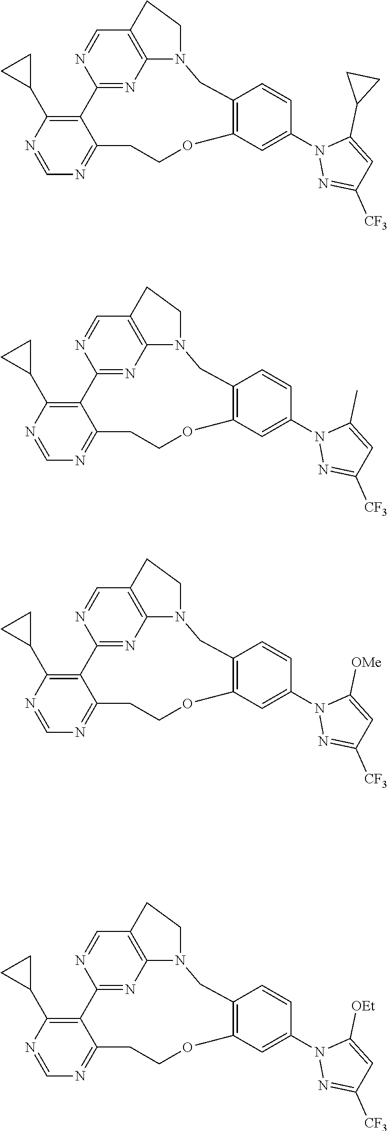 [SMILES: FC(F)(F)c1cc(C2CC2)n(-c2ccc3c(c2)OCCc2ncnc(C4CC4)c2-c2ncc4c(n2)N(CC4)C3)n1 ; Cc1cc(C(F)(F)F)nn1-c1ccc2c(c1)OCCc1ncnc(C3CC3)c1-c1ncc3c(n1)N(CC3)C2 ; COc1cc(C(F)(F)F)nn1-c1ccc2c(c1)OCCc1ncnc(C3CC3)c1-c1ncc3c(n1)N(CC3)C2 ; CCOc1cc(C(F)(F)F)nn1-c1ccc2c(c1)OCCc1ncnc(C3CC3)c1-c1ncc3c(n1)N(CC3)C2]

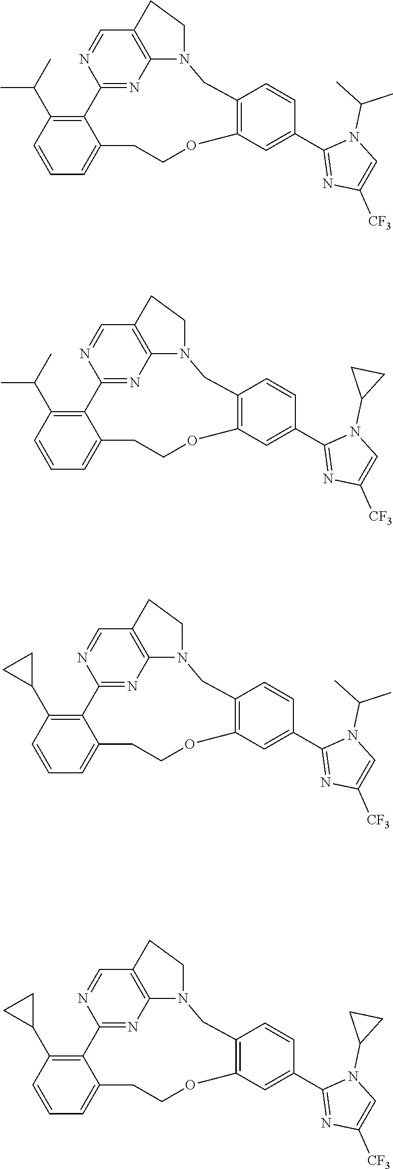

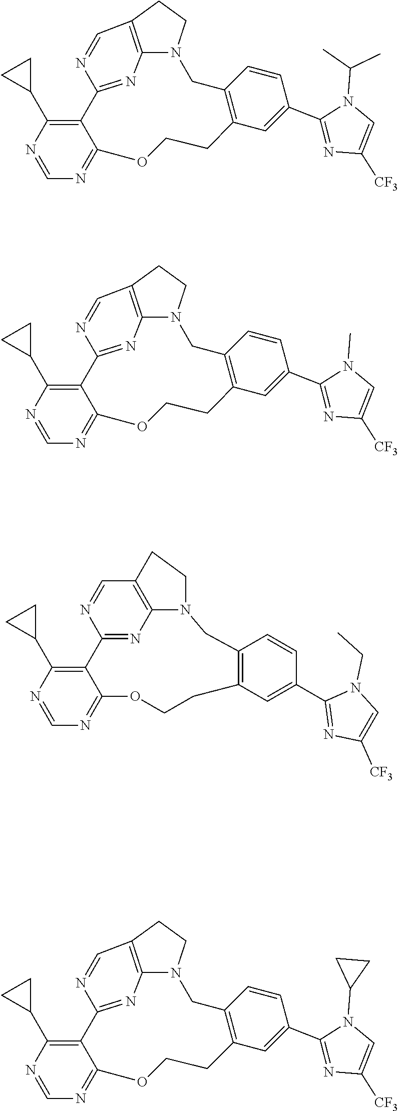 [SMILES: CC(C)n1cc(C(F)(F)F)nc1-c1ccc2c(c1)CCOc1ncnc(C3CC3)c1-c1ncc3c(n1)N(CC3)C2 ; Cn1cc(C(F)(F)F)nc1-c1ccc2c(c1)CCOc1ncnc(C3CC3)c1-c1ncc3c(n1)N(CC3)C2 ; CCn1cc(C(F)(F)F)nc1-c1ccc2c(c1)CCOc1ncnc(C3CC3)c1-c1ncc3c(n1)N(CC3)C2 ; FC(F)(F)c1cn(C2CC2)c(-c2ccc3c(c2)CCOc2ncnc(C4CC4)c2-c2ncc4c(n2)N(CC4)C3)n1]

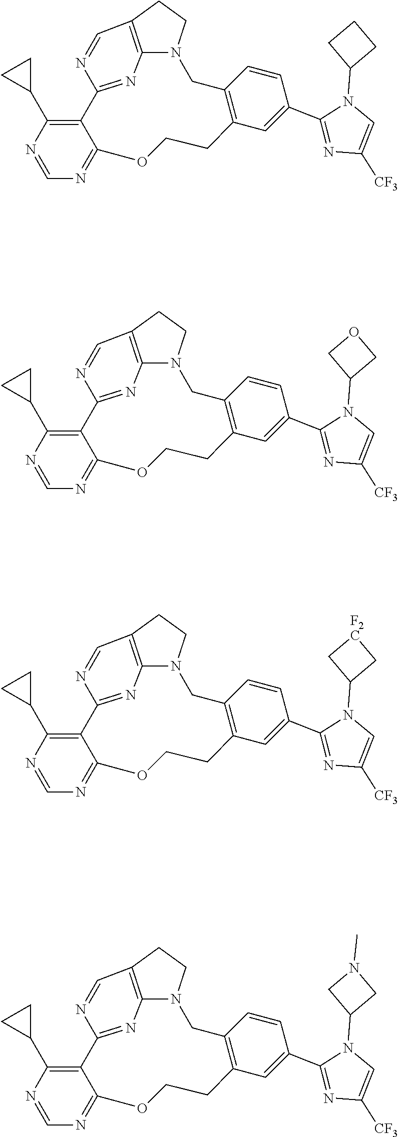 [SMILES: FC(F)(F)c1cn(C2CCC2)c(-c2ccc3c(c2)CCOc2ncnc(C4CC4)c2-c2ncc4c(n2)N(CC4)C3)n1 ; FC(F)(F)c1cn(C2COC2)c(-c2ccc3c(c2)CCOc2ncnc(C4CC4)c2-c2ncc4c(n2)N(CC4)C3)n1 ; FC1(F)CC(n2cc(C(F)(F)F)nc2-c2ccc3c(c2)CCOc2ncnc(C4CC4)c2-c2ncc4c(n2)N(CC4)C3)C1 ; CN1CC(n2cc(C(F)(F)F)nc2-c2ccc3c(c2)CCOc2ncnc(C4CC4)c2-c2ncc4c(n2)N(CC4)C3)C1]

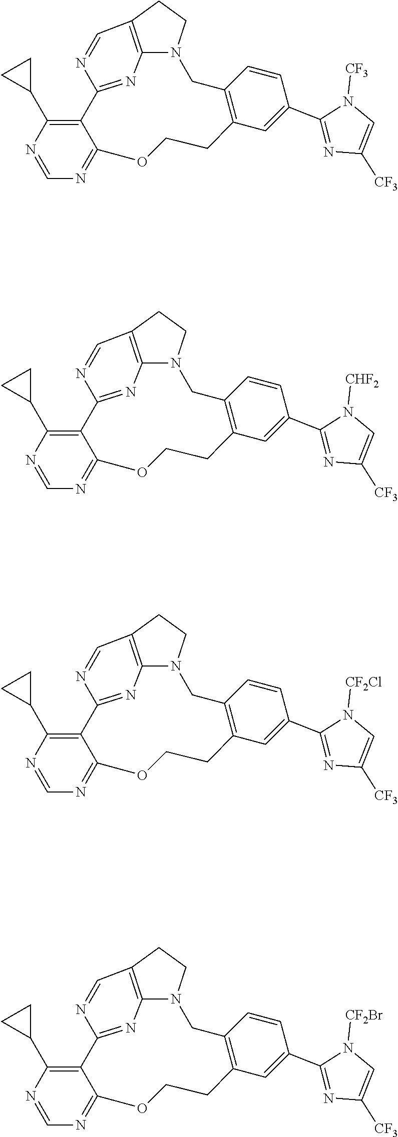

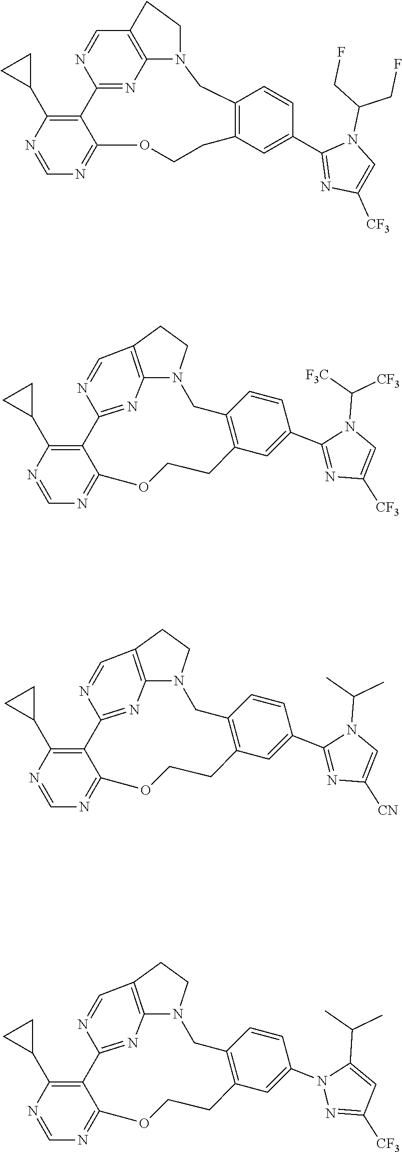 [SMILES: FCC(CF)n1cc(C(F)(F)F)nc1-c1ccc2c(c1)CCOc1ncnc(C3CC3)c1-c1ncc3c(n1)N(CC3)C2 ; FC(F)(F)c1cn(C(C(F)(F)F)C(F)(F)F)c(-c2ccc3c(c2)CCOc2ncnc(C4CC4)c2-c2ncc4c(n2)N(CC4)C3)n1 ; CC(C)n1cc(C#N)nc1-c1ccc2c(c1)CCOc1ncnc(C3CC3)c1-c1ncc3c(n1)N(CC3)C2 ; CC(C)c1cc(C(F)(F)F)nn1-c1ccc2c(c1)CCOc1ncnc(C3CC3)c1-c1ncc3c(n1)N(CC3)C2]

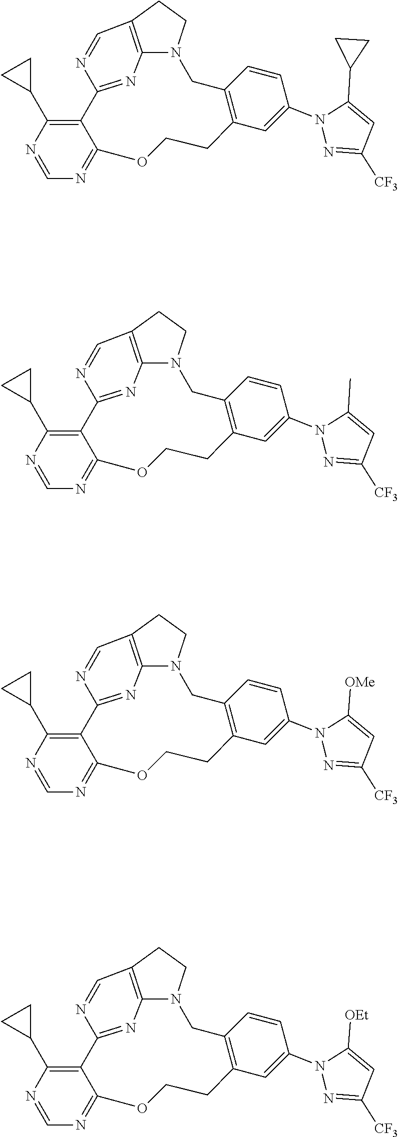 [SMILES: FC(F)(F)c1cc(C2CC2)n(-c2ccc3c(c2)CCOc2ncnc(C4CC4)c2-c2ncc4c(n2)N(CC4)C3)n1 ; Cc1cc(C(F)(F)F)nn1-c1ccc2c(c1)CCOc1ncnc(C3CC3)c1-c1ncc3c(n1)N(CC3)C2 ; COc1cc(C(F)(F)F)nn1-c1ccc2c(c1)CCOc1ncnc(C3CC3)c1-c1ncc3c(n1)N(CC3)C2 ; CCOc1cc(C(F)(F)F)nn1-c1ccc2c(c1)CCOc1ncnc(C3CC3)c1-c1ncc3c(n1)N(CC3)C2]

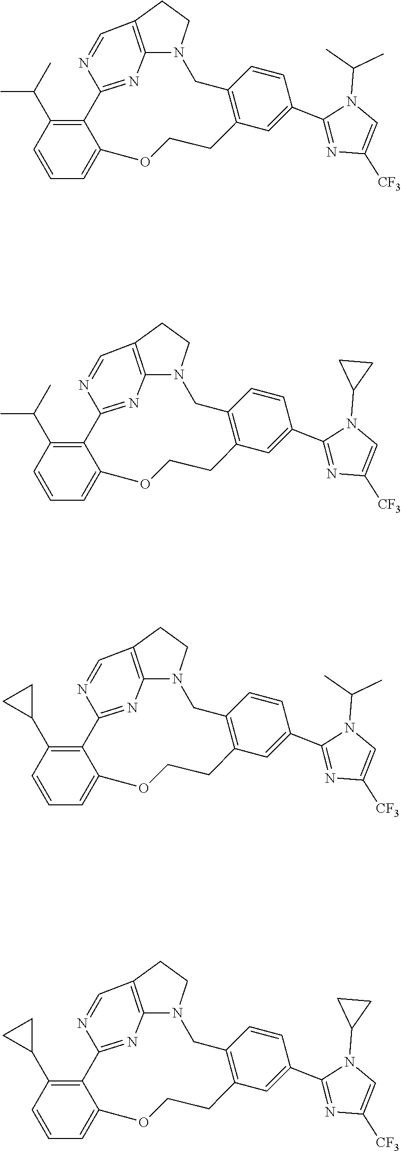

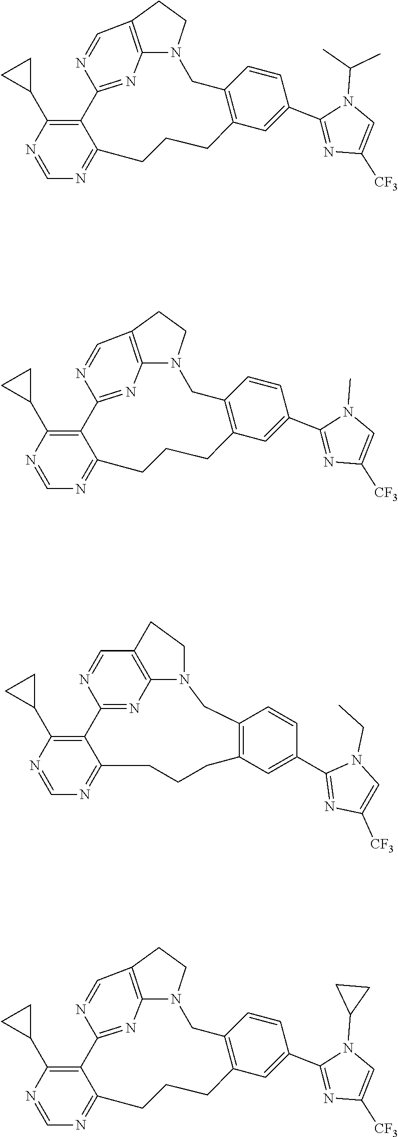 [SMILES: CC(C)n1cc(C(F)(F)F)nc1-c1ccc2c(c1)CCCc1ncnc(C3CC3)c1-c1ncc3c(n1)N(CC3)C2 ; Cn1cc(C(F)(F)F)nc1-c1ccc2c(c1)CCCc1ncnc(C3CC3)c1-c1ncc3c(n1)N(CC3)C2 ; CCn1cc(C(F)(F)F)nc1-c1ccc2c(c1)CCCc1ncnc(C3CC3)c1-c1ncc3c(n1)N(CC3)C2 ; FC(F)(F)c1cn(C2CC2)c(-c2ccc3c(c2)CCCc2ncnc(C4CC4)c2-c2ncc4c(n2)N(CC4)C3)n1]

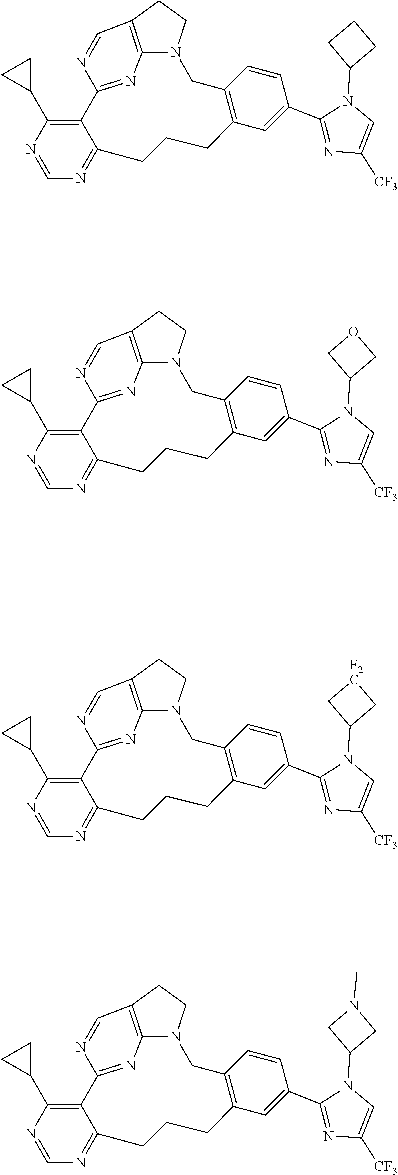 [SMILES: FC(F)(F)c1cn(C2CCC2)c(-c2ccc3c(c2)CCCc2ncnc(C4CC4)c2-c2ncc4c(n2)N(CC4)C3)n1 ; FC(F)(F)c1cn(C2COC2)c(-c2ccc3c(c2)CCCc2ncnc(C4CC4)c2-c2ncc4c(n2)N(CC4)C3)n1 ; FC1(F)CC(n2cc(C(F)(F)F)nc2-c2ccc3c(c2)CCCc2ncnc(C4CC4)c2-c2ncc4c(n2)N(CC4)C3)C1 ; CN1CC(n2cc(C(F)(F)F)nc2-c2ccc3c(c2)CCCc2ncnc(C4CC4)c2-c2ncc4c(n2)N(CC4)C3)C1]

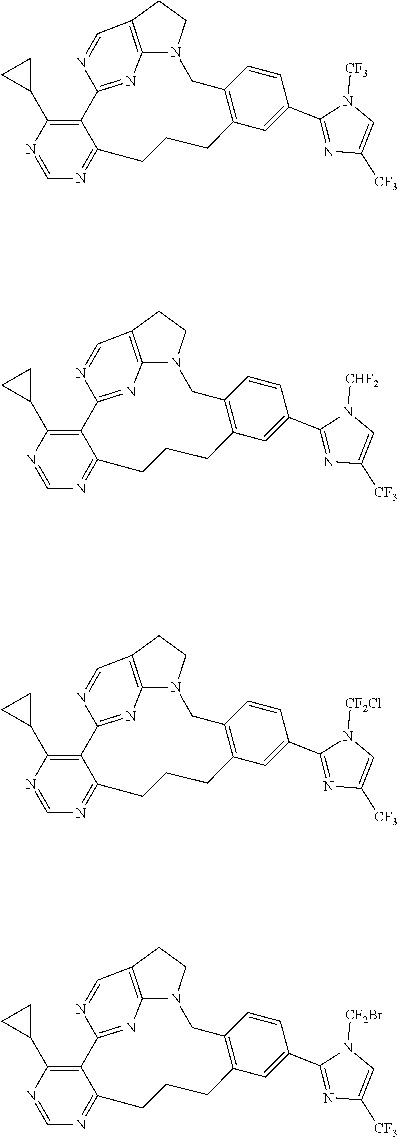

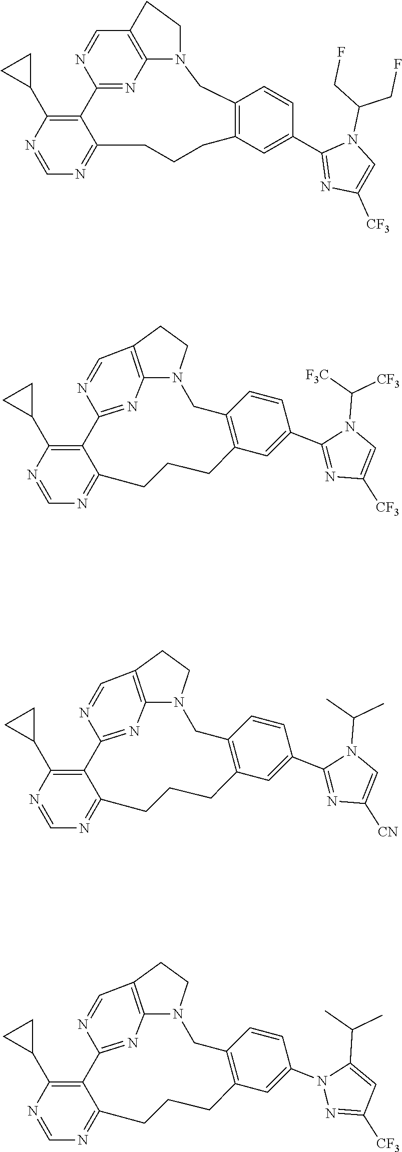 [SMILES: FCC(CF)n1cc(C(F)(F)F)nc1-c1ccc2c(c1)CCCc1ncnc(C3CC3)c1-c1ncc3c(n1)N(CC3)C2 ; FC(F)(F)c1cn(C(C(F)(F)F)C(F)(F)F)c(-c2ccc3c(c2)CCCc2ncnc(C4CC4)c2-c2ncc4c(n2)N(CC4)C3)n1 ; CC(C)n1cc(C#N)nc1-c1ccc2c(c1)CCCc1ncnc(C3CC3)c1-c1ncc3c(n1)N(CC3)C2 ; CC(C)c1cc(C(F)(F)F)nn1-c1ccc2c(c1)CCCc1ncnc(C3CC3)c1-c1ncc3c(n1)N(CC3)C2]

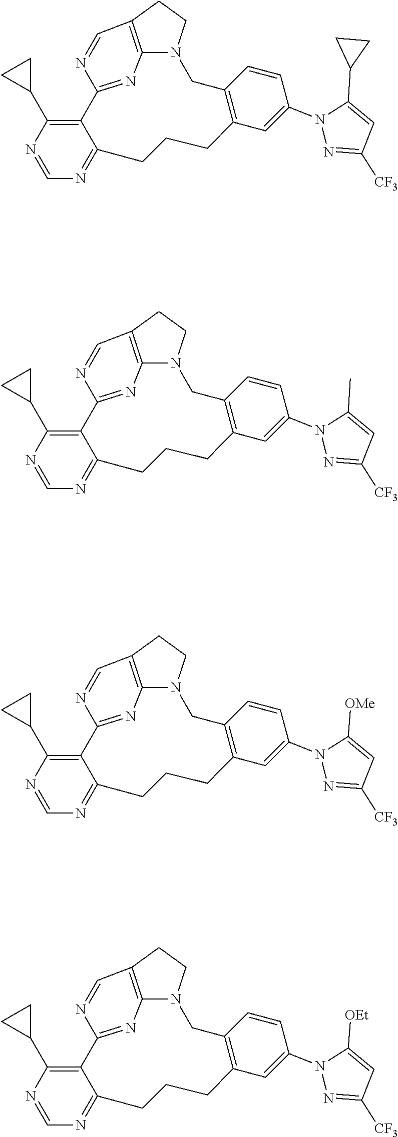 [SMILES: FC(F)(F)c1cc(C2CC2)n(-c2ccc3c(c2)CCCc2ncnc(C4CC4)c2-c2ncc4c(n2)N(CC4)C3)n1 ; Cc1cc(C(F)(F)F)nn1-c1ccc2c(c1)CCCc1ncnc(C3CC3)c1-c1ncc3c(n1)N(CC3)C2 ; COc1cc(C(F)(F)F)nn1-c1ccc2c(c1)CCCc1ncnc(C3CC3)c1-c1ncc3c(n1)N(CC3)C2 ; CCOc1cc(C(F)(F)F)nn1-c1ccc2c(c1)CCCc1ncnc(C3CC3)c1-c1ncc3c(n1)N(CC3)C2]

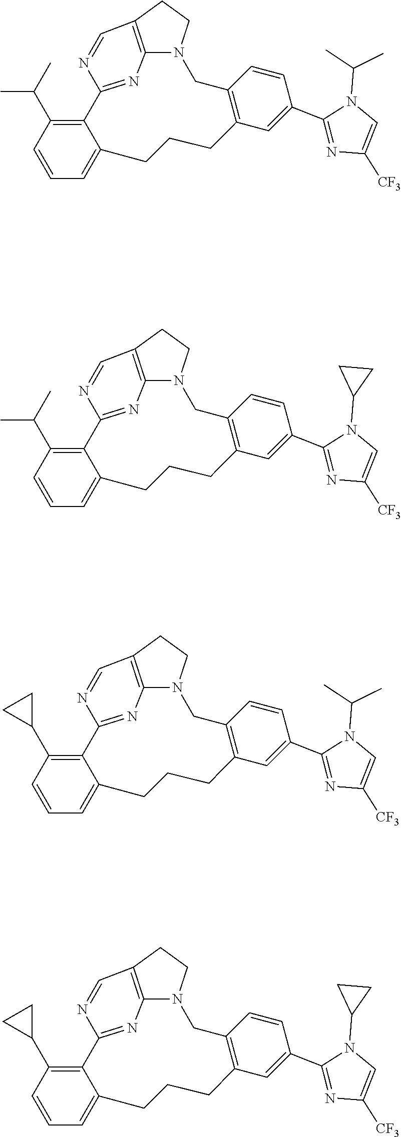

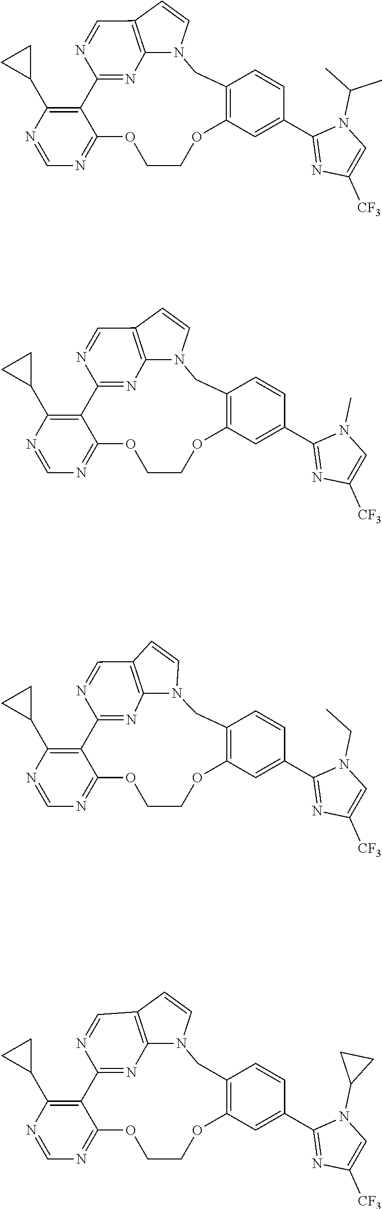 [SMILES: CC(C)n1cc(C(F)(F)F)nc1-c1ccc2c(c1)OCCOc1ncnc(C3CC3)c1-c1ncc3ccn(c3n1)C2 ; Cn1cc(C(F)(F)F)nc1-c1ccc2c(c1)OCCOc1ncnc(C3CC3)c1-c1ncc3ccn(c3n1)C2 ; CCn1cc(C(F)(F)F)nc1-c1ccc2c(c1)OCCOc1ncnc(C3CC3)c1-c1ncc3ccn(c3n1)C2 ; FC(F)(F)c1cn(C2CC2)c(-c2ccc3c(c2)OCCOc2ncnc(C4CC4)c2-c2ncc4ccn(c4n2)C3)n1]

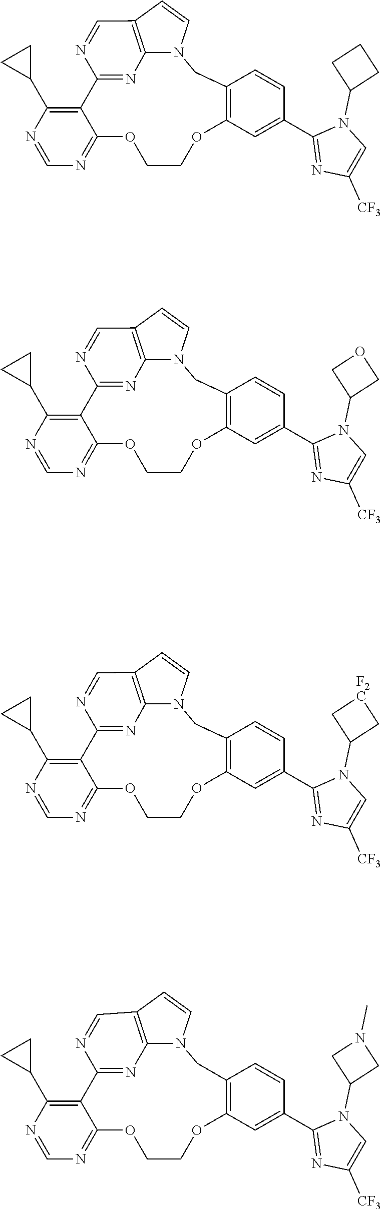 [SMILES: FC(F)(F)c1cn(C2CCC2)c(-c2ccc3c(c2)OCCOc2ncnc(C4CC4)c2-c2ncc4ccn(c4n2)C3)n1 ; FC(F)(F)c1cn(C2COC2)c(-c2ccc3c(c2)OCCOc2ncnc(C4CC4)c2-c2ncc4ccn(c4n2)C3)n1 ; FC1(F)CC(n2cc(C(F)(F)F)nc2-c2ccc3c(c2)OCCOc2ncnc(C4CC4)c2-c2ncc4ccn(c4n2)C3)C1 ; CN1CC(n2cc(C(F)(F)F)nc2-c2ccc3c(c2)OCCOc2ncnc(C4CC4)c2-c2ncc4ccn(c4n2)C3)C1]

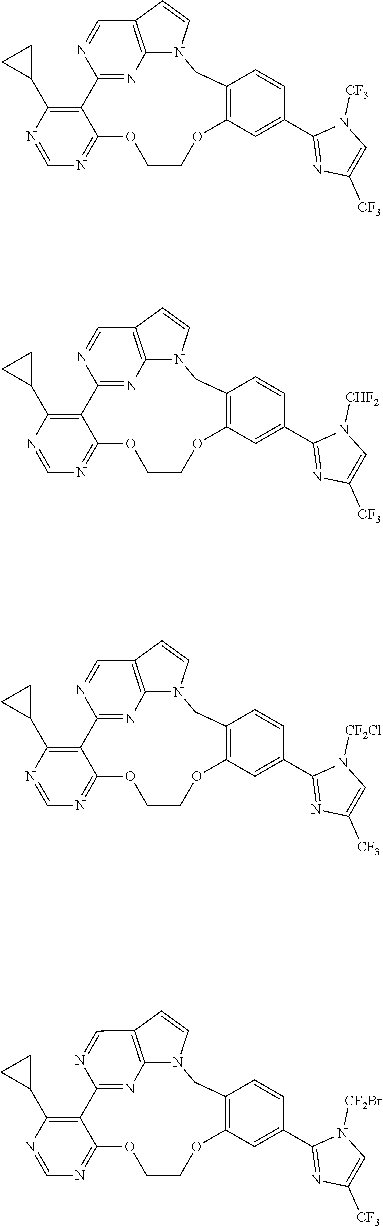

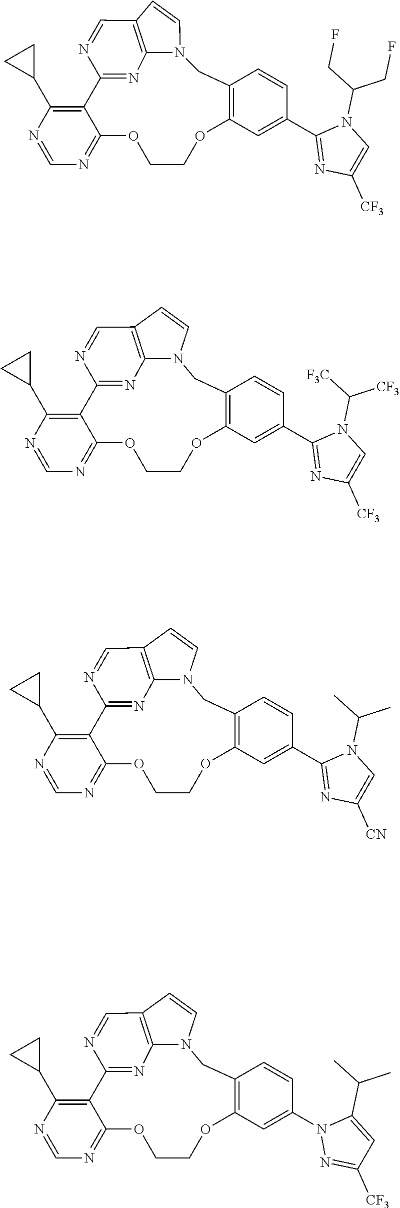 [SMILES: FCC(CF)n1cc(C(F)(F)F)nc1-c1ccc2c(c1)OCCOc1ncnc(C3CC3)c1-c1ncc3ccn(c3n1)C2 ; FC(F)(F)c1cn(C(C(F)(F)F)C(F)(F)F)c(-c2ccc3c(c2)OCCOc2ncnc(C4CC4)c2-c2ncc4ccn(c4n2)C3)n1 ; CC(C)n1cc(C#N)nc1-c1ccc2c(c1)OCCOc1ncnc(C3CC3)c1-c1ncc3ccn(c3n1)C2 ; CC(C)c1cc(C(F)(F)F)nn1-c1ccc2c(c1)OCCOc1ncnc(C3CC3)c1-c1ncc3ccn(c3n1)C2]

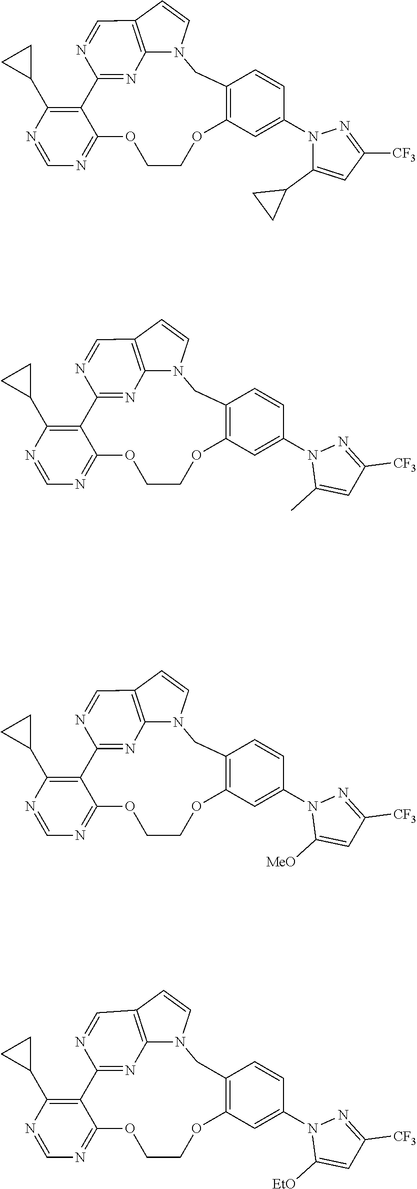 [SMILES: FC(F)(F)c1cc(C2CC2)n(-c2ccc3c(c2)OCCOc2ncnc(C4CC4)c2-c2ncc4ccn(c4n2)C3)n1 ; Cc1cc(C(F)(F)F)nn1-c1ccc2c(c1)OCCOc1ncnc(C3CC3)c1-c1ncc3ccn(c3n1)C2 ; COc1cc(C(F)(F)F)nn1-c1ccc2c(c1)OCCOc1ncnc(C3CC3)c1-c1ncc3ccn(c3n1)C2 ; CCOc1cc(C(F)(F)F)nn1-c1ccc2c(c1)OCCOc1ncnc(C3CC3)c1-c1ncc3ccn(c3n1)C2]

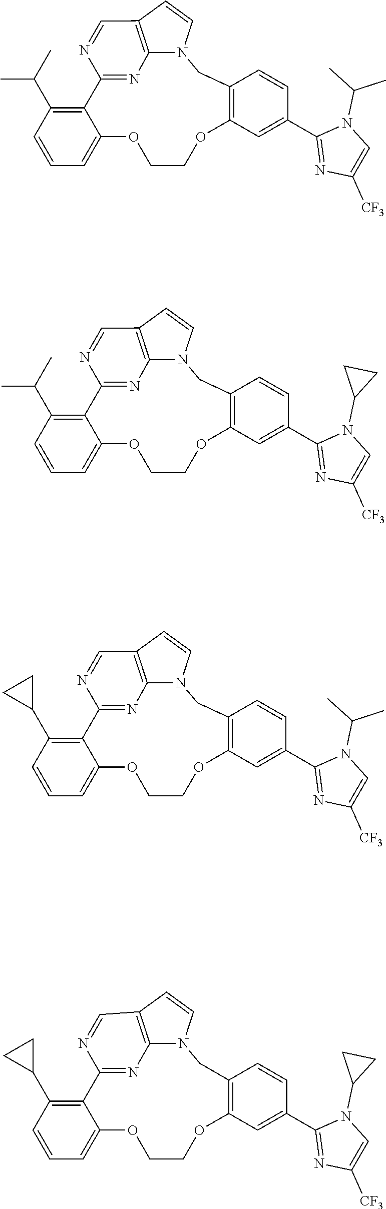

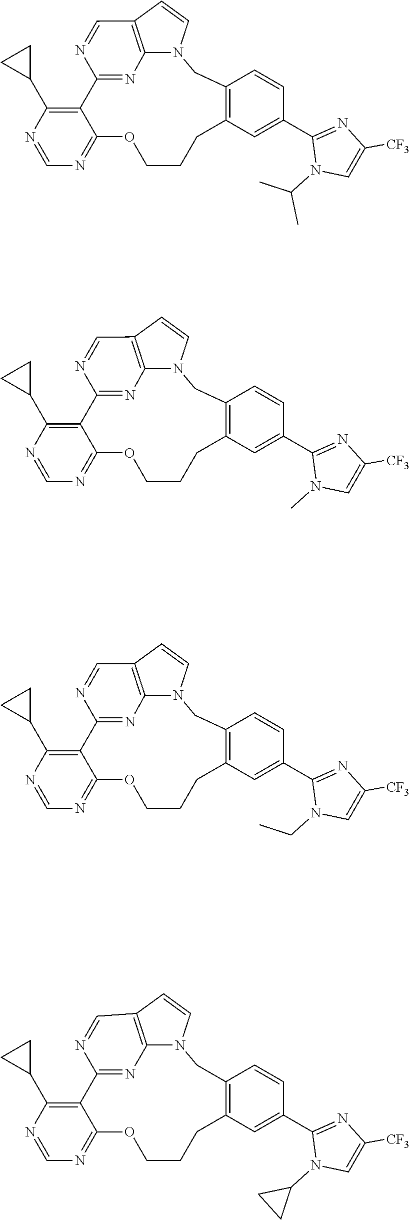 [SMILES: CC(C)n1cc(C(F)(F)F)nc1-c1ccc2c(c1)CCCOc1ncnc(C3CC3)c1-c1ncc3ccn(c3n1)C2 ; Cn1cc(C(F)(F)F)nc1-c1ccc2c(c1)CCCOc1ncnc(C3CC3)c1-c1ncc3ccn(c3n1)C2 ; CCn1cc(C(F)(F)F)nc1-c1ccc2c(c1)CCCOc1ncnc(C3CC3)c1-c1ncc3ccn(c3n1)C2 ; FC(F)(F)c1cn(C2CC2)c(-c2ccc3c(c2)CCCOc2ncnc(C4CC4)c2-c2ncc4ccn(c4n2)C3)n1]

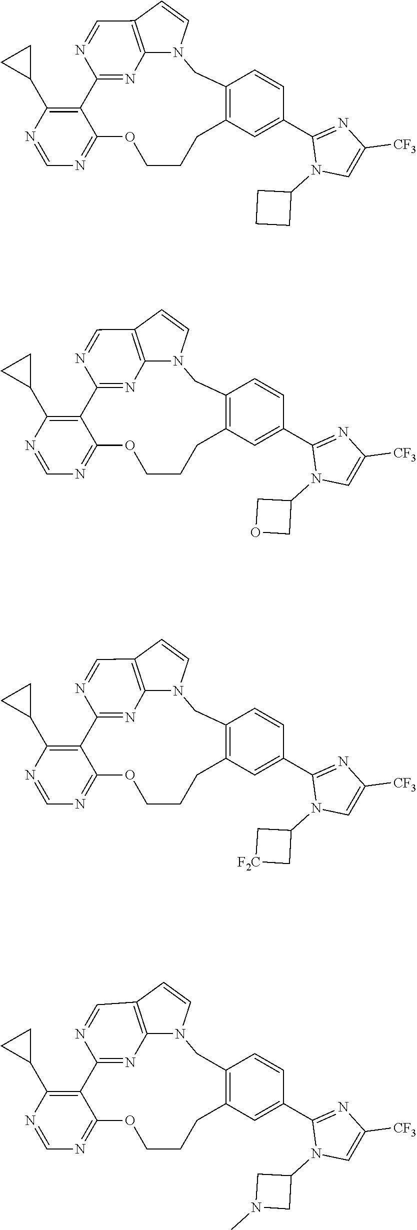 [SMILES: FC(F)(F)c1cn(C2CCC2)c(-c2ccc3c(c2)CCCOc2ncnc(C4CC4)c2-c2ncc4ccn(c4n2)C3)n1 ; FC(F)(F)c1cn(C2COC2)c(-c2ccc3c(c2)CCCOc2ncnc(C4CC4)c2-c2ncc4ccn(c4n2)C3)n1 ; FC1(F)CC(n2cc(C(F)(F)F)nc2-c2ccc3c(c2)CCCOc2ncnc(C4CC4)c2-c2ncc4ccn(c4n2)C3)C1 ; CN1CC(n2cc(C(F)(F)F)nc2-c2ccc3c(c2)CCCOc2ncnc(C4CC4)c2-c2ncc4ccn(c4n2)C3)C1]

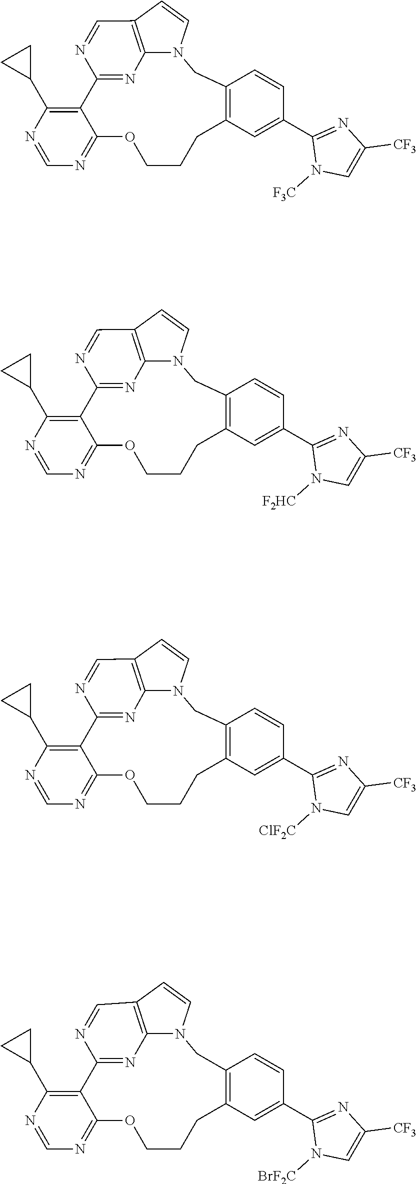

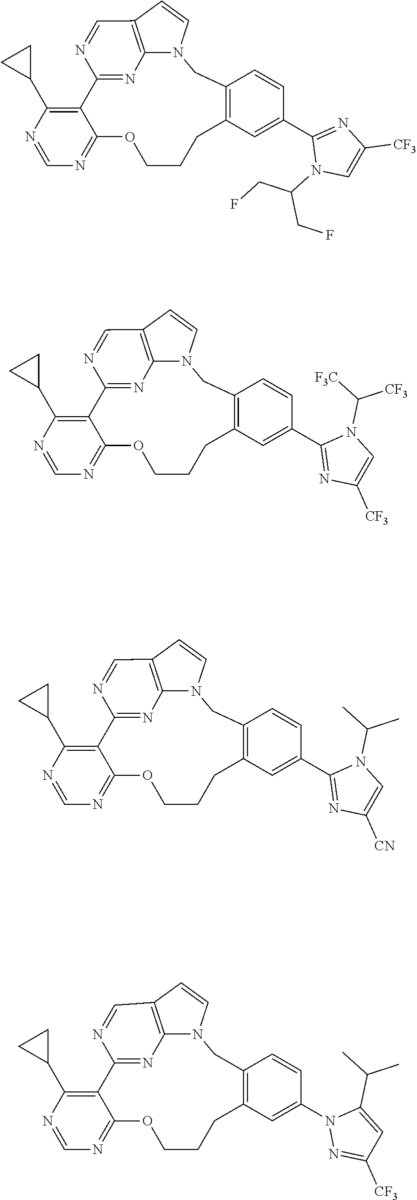 [SMILES: FCC(CF)n1cc(C(F)(F)F)nc1-c1ccc2c(c1)CCCOc1ncnc(C3CC3)c1-c1ncc3ccn(c3n1)C2 ; FC(F)(F)c1cn(C(C(F)(F)F)C(F)(F)F)c(-c2ccc3c(c2)CCCOc2ncnc(C4CC4)c2-c2ncc4ccn(c4n2)C3)n1 ; CC(C)n1cc(C#N)nc1-c1ccc2c(c1)CCCOc1ncnc(C3CC3)c1-c1ncc3ccn(c3n1)C2 ; CC(C)c1cc(C(F)(F)F)nn1-c1ccc2c(c1)CCCOc1ncnc(C3CC3)c1-c1ncc3ccn(c3n1)C2]

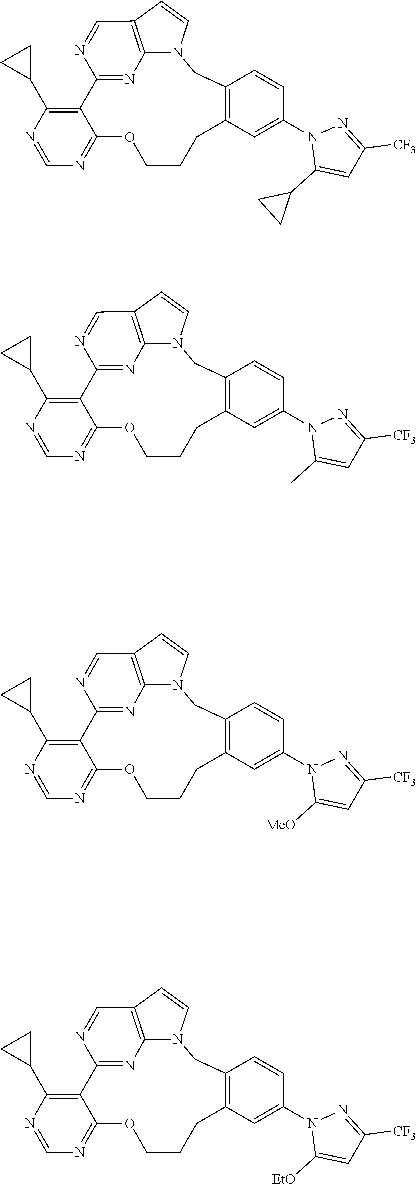 [SMILES: FC(F)(F)c1cc(C2CC2)n(-c2ccc3c(c2)CCCOc2ncnc(C4CC4)c2-c2ncc4ccn(c4n2)C3)n1 ; Cc1cc(C(F)(F)F)nn1-c1ccc2c(c1)CCCOc1ncnc(C3CC3)c1-c1ncc3ccn(c3n1)C2 ; COc1cc(C(F)(F)F)nn1-c1ccc2c(c1)CCCOc1ncnc(C3CC3)c1-c1ncc3ccn(c3n1)C2 ; CCOc1cc(C(F)(F)F)nn1-c1ccc2c(c1)CCCOc1ncnc(C3CC3)c1-c1ncc3ccn(c3n1)C2]

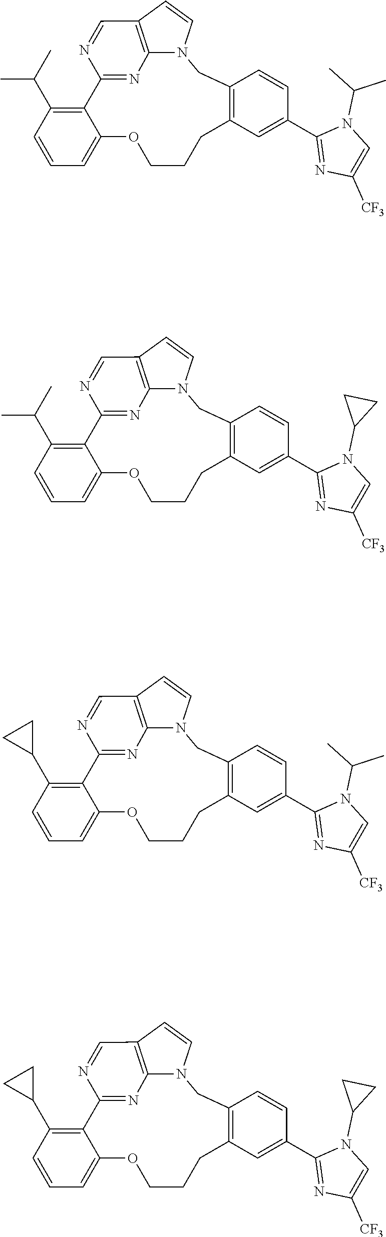

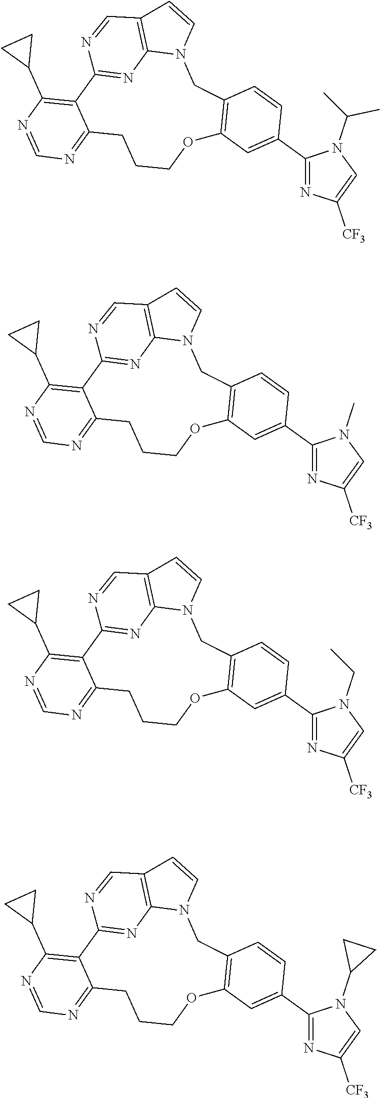 [SMILES: CC(C)n1cc(C(F)(F)F)nc1-c1ccc2c(c1)OCCCc1ncnc(C3CC3)c1-c1ncc3ccn(c3n1)C2 ; Cn1cc(C(F)(F)F)nc1-c1ccc2c(c1)OCCCc1ncnc(C3CC3)c1-c1ncc3ccn(c3n1)C2 ; CCn1cc(C(F)(F)F)nc1-c1ccc2c(c1)OCCCc1ncnc(C3CC3)c1-c1ncc3ccn(c3n1)C2 ; FC(F)(F)c1cn(C2CC2)c(-c2ccc3c(c2)OCCCc2ncnc(C4CC4)c2-c2ncc4ccn(c4n2)C3)n1]

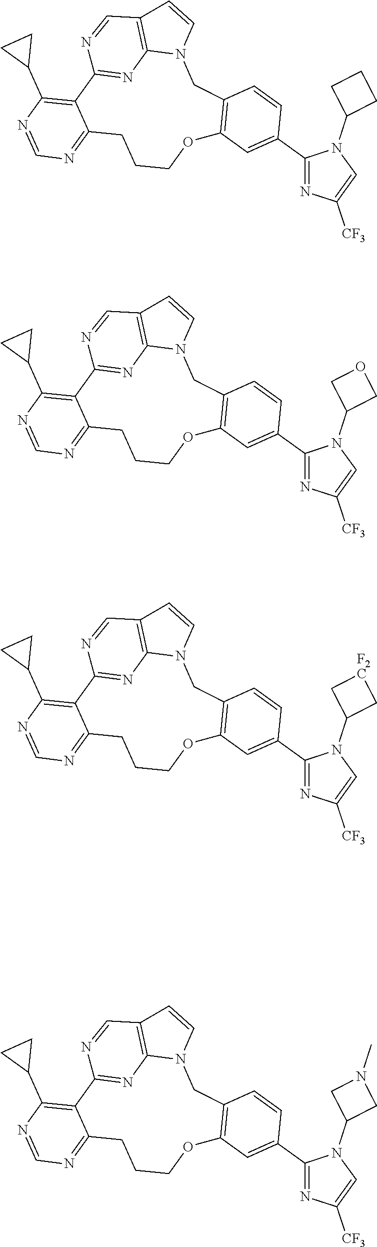 [SMILES: FC(F)(F)c1cn(C2CCC2)c(-c2ccc3c(c2)OCCCc2ncnc(C4CC4)c2-c2ncc4ccn(c4n2)C3)n1 ; FC(F)(F)c1cn(C2COC2)c(-c2ccc3c(c2)OCCCc2ncnc(C4CC4)c2-c2ncc4ccn(c4n2)C3)n1 ; FC1(F)CC(n2cc(C(F)(F)F)nc2-c2ccc3c(c2)OCCCc2ncnc(C4CC4)c2-c2ncc4ccn(c4n2)C3)C1 ; CN1CC(n2cc(C(F)(F)F)nc2-c2ccc3c(c2)OCCCc2ncnc(C4CC4)c2-c2ncc4ccn(c4n2)C3)C1]

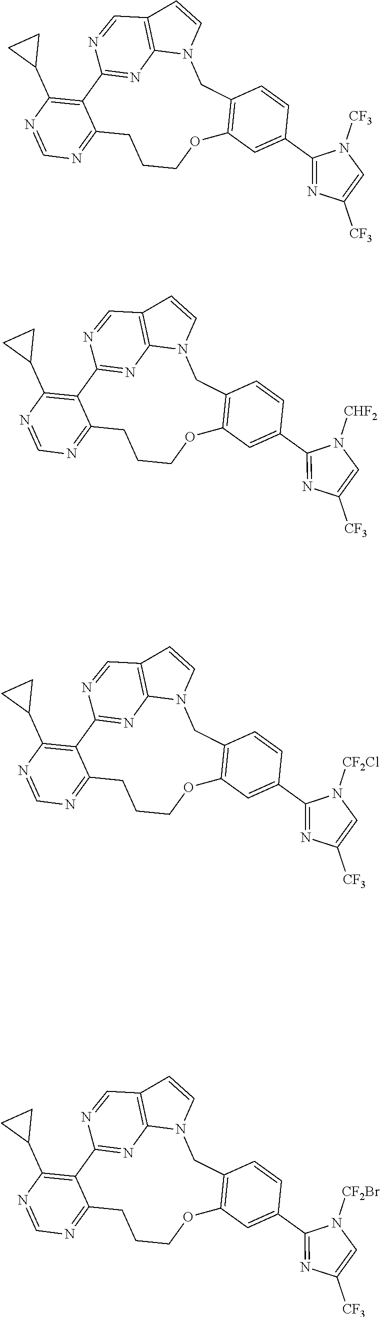

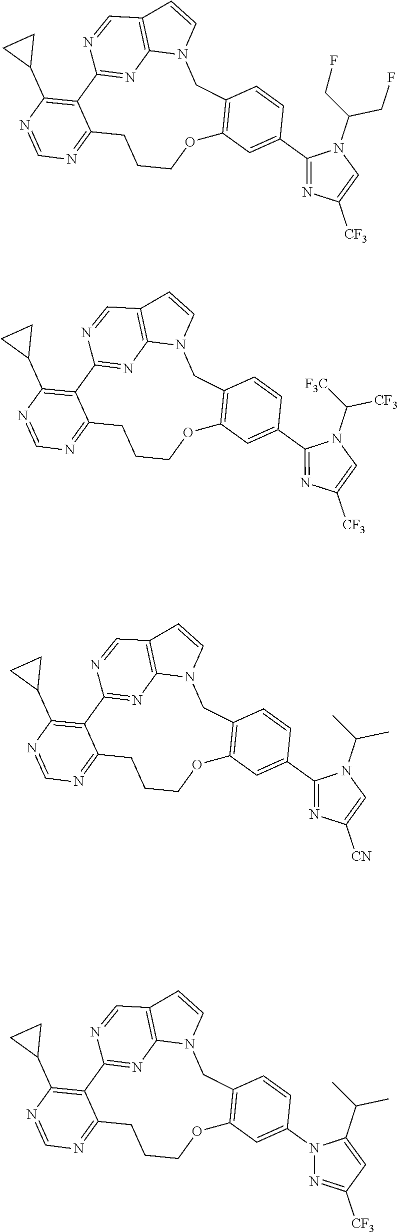 [SMILES: FCC(CF)n1cc(C(F)(F)F)nc1-c1ccc2c(c1)OCCCc1ncnc(C3CC3)c1-c1ncc3ccn(c3n1)C2 ; FC(F)(F)c1cn(C(C(F)(F)F)C(F)(F)F)c(-c2ccc3c(c2)OCCCc2ncnc(C4CC4)c2-c2ncc4ccn(c4n2)C3)n1 ; CC(C)n1cc(C#N)nc1-c1ccc2c(c1)OCCCc1ncnc(C3CC3)c1-c1ncc3ccn(c3n1)C2 ; CC(C)c1cc(C(F)(F)F)nn1-c1ccc2c(c1)OCCCc1ncnc(C3CC3)c1-c1ncc3ccn(c3n1)C2]

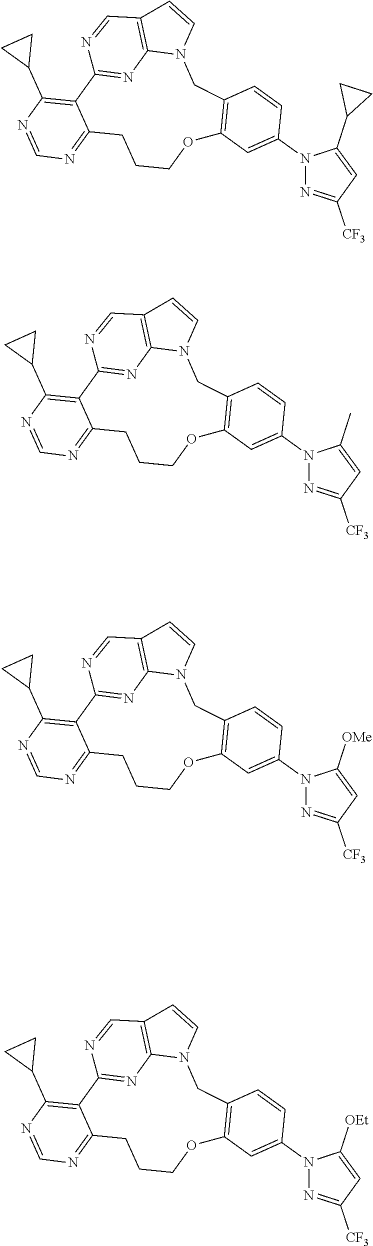 [SMILES: FC(F)(F)c1cc(C2CC2)n(-c2ccc3c(c2)OCCCc2ncnc(C4CC4)c2-c2ncc4ccn(c4n2)C3)n1 ; Cc1cc(C(F)(F)F)nn1-c1ccc2c(c1)OCCCc1ncnc(C3CC3)c1-c1ncc3ccn(c3n1)C2 ; COc1cc(C(F)(F)F)nn1-c1ccc2c(c1)OCCCc1ncnc(C3CC3)c1-c1ncc3ccn(c3n1)C2 ; CCOc1cc(C(F)(F)F)nn1-c1ccc2c(c1)OCCCc1ncnc(C3CC3)c1-c1ncc3ccn(c3n1)C2]

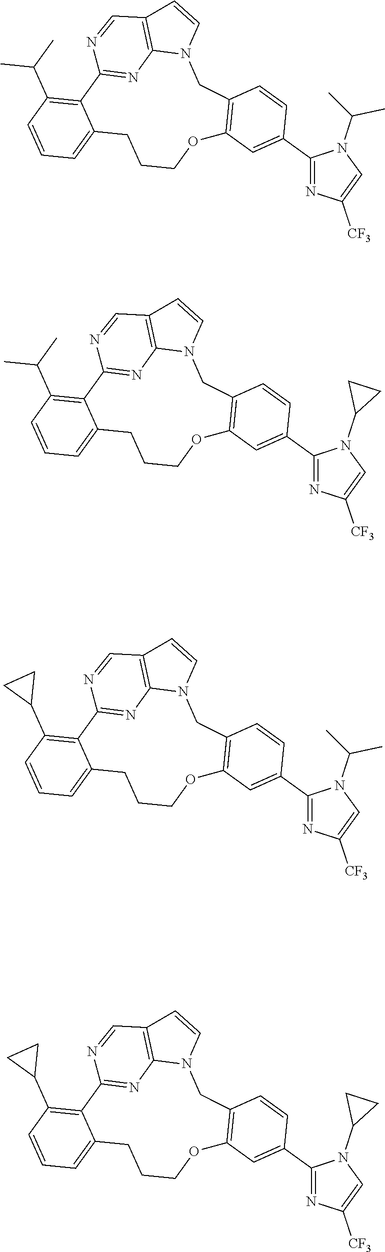

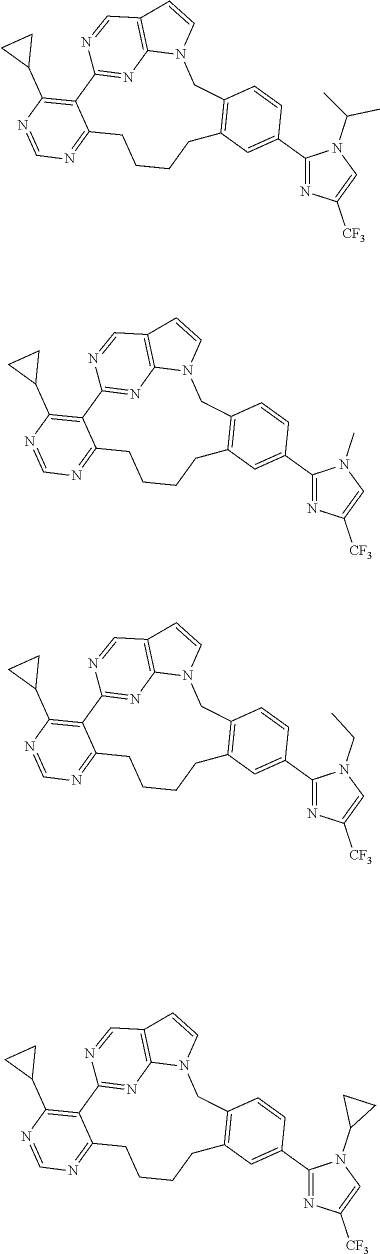 [SMILES: CC(C)n1cc(C(F)(F)F)nc1-c1ccc2c(c1)CCCCc1ncnc(C3CC3)c1-c1ncc3ccn(c3n1)C2 ; Cn1cc(C(F)(F)F)nc1-c1ccc2c(c1)CCCCc1ncnc(C3CC3)c1-c1ncc3ccn(c3n1)C2 ; CCn1cc(C(F)(F)F)nc1-c1ccc2c(c1)CCCCc1ncnc(C3CC3)c1-c1ncc3ccn(c3n1)C2 ; FC(F)(F)c1cn(C2CC2)c(-c2ccc3c(c2)CCCCc2ncnc(C4CC4)c2-c2ncc4ccn(c4n2)C3)n1]

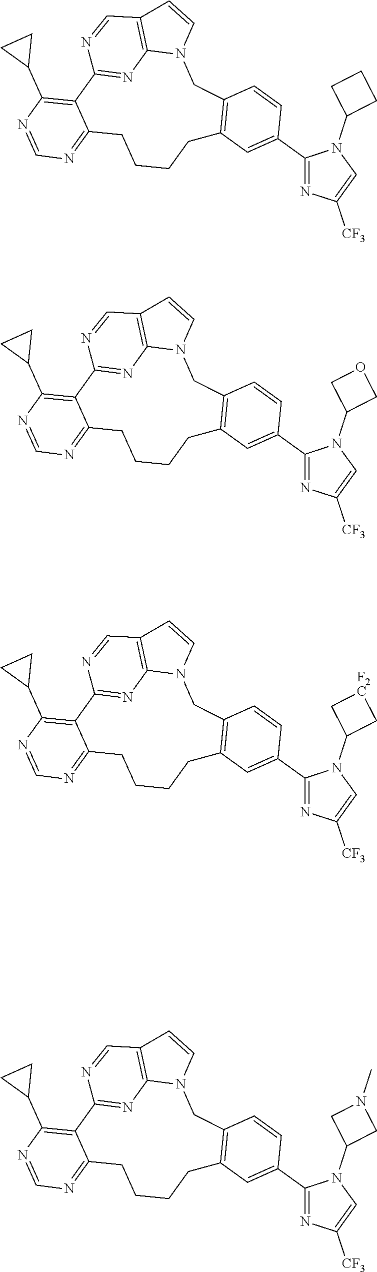 [SMILES: FC(F)(F)c1cn(C2CCC2)c(-c2ccc3c(c2)CCCCc2ncnc(C4CC4)c2-c2ncc4ccn(c4n2)C3)n1 ; FC(F)(F)c1cn(C2COC2)c(-c2ccc3c(c2)CCCCc2ncnc(C4CC4)c2-c2ncc4ccn(c4n2)C3)n1 ; FC1(F)CC(n2cc(C(F)(F)F)nc2-c2ccc3c(c2)CCCCc2ncnc(C4CC4)c2-c2ncc4ccn(c4n2)C3)C1 ; CN1CC(n2cc(C(F)(F)F)nc2-c2ccc3c(c2)CCCCc2ncnc(C4CC4)c2-c2ncc4ccn(c4n2)C3)C1]

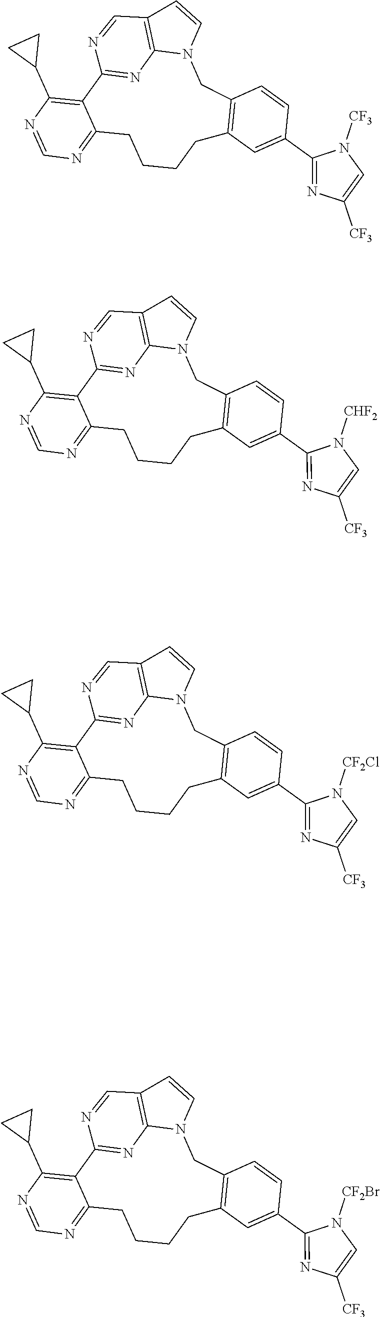

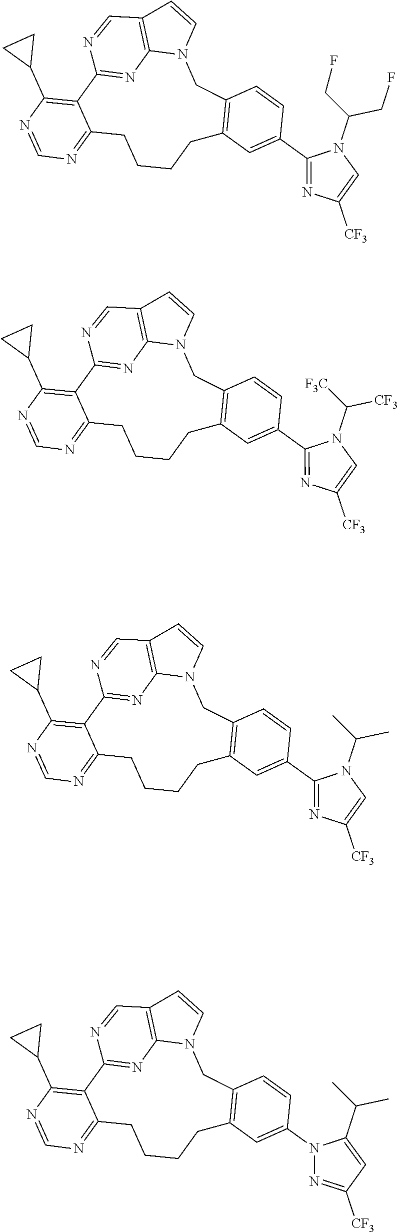 [SMILES: FCC(CF)n1cc(C(F)(F)F)nc1-c1ccc2c(c1)CCCCc1ncnc(C3CC3)c1-c1ncc3ccn(c3n1)C2 ; FC(F)(F)c1cn(C(C(F)(F)F)C(F)(F)F)c(-c2ccc3c(c2)CCCCc2ncnc(C4CC4)c2-c2ncc4ccn(c4n2)C3)n1 ; CC(C)n1cc(C(F)(F)F)nc1-c1ccc2c(c1)CCCCc1ncnc(C3CC3)c1-c1ncc3ccn(c3n1)C2 ; CC(C)c1cc(C(F)(F)F)nn1-c1ccc2c(c1)CCCCc1ncnc(C3CC3)c1-c1ncc3ccn(c3n1)C2]

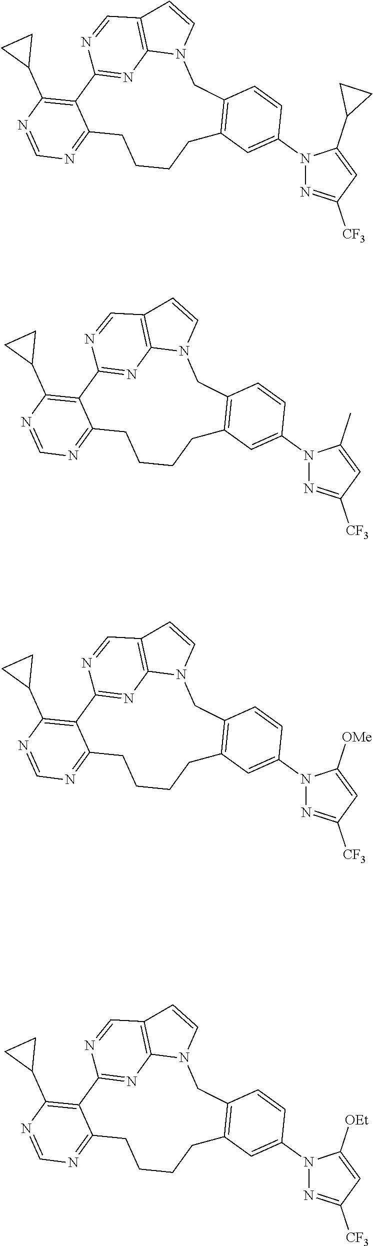 [SMILES: FC(F)(F)c1cc(C2CC2)n(-c2ccc3c(c2)CCCCc2ncnc(C4CC4)c2-c2ncc4ccn(c4n2)C3)n1 ; Cc1cc(C(F)(F)F)nn1-c1ccc2c(c1)CCCCc1ncnc(C3CC3)c1-c1ncc3ccn(c3n1)C2 ; COc1cc(C(F)(F)F)nn1-c1ccc2c(c1)CCCCc1ncnc(C3CC3)c1-c1ncc3ccn(c3n1)C2 ; CCOc1cc(C(F)(F)F)nn1-c1ccc2c(c1)CCCCc1ncnc(C3CC3)c1-c1ncc3ccn(c3n1)C2]

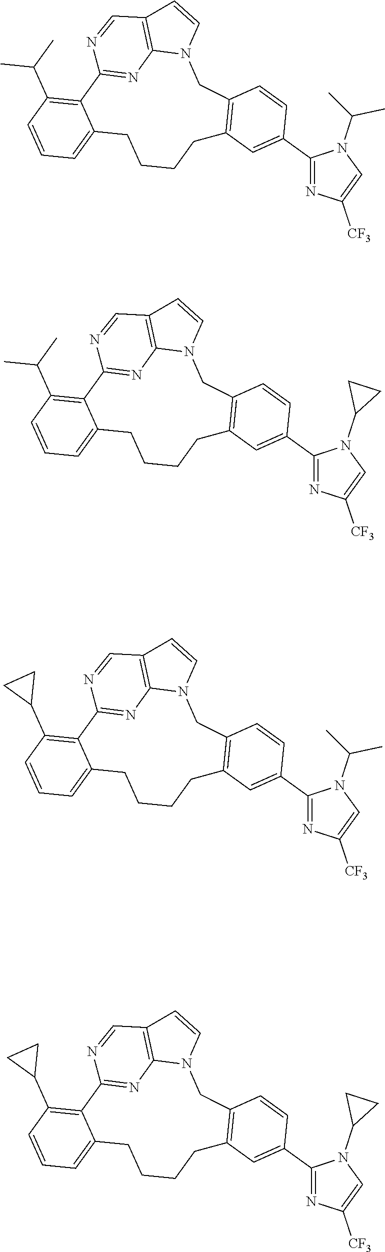

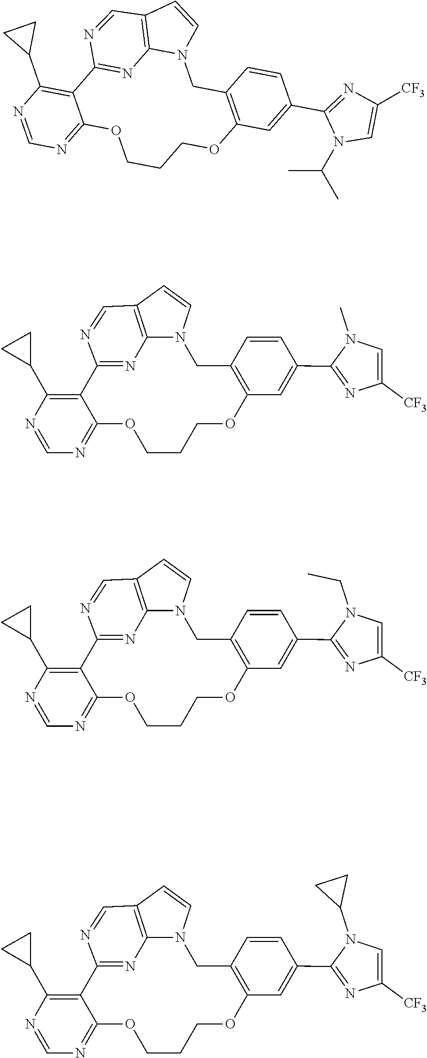 [SMILES: CC(C)n1cc(C(F)(F)F)nc1-c1ccc2c(c1)OCCCOc1ncnc(C3CC3)c1-c1ncc3ccn(c3n1)C2 ; Cn1cc(C(F)(F)F)nc1-c1ccc2c(c1)OCCCOc1ncnc(C3CC3)c1-c1ncc3ccn(c3n1)C2 ; CCn1cc(C(F)(F)F)nc1-c1ccc2c(c1)OCCCOc1ncnc(C3CC3)c1-c1ncc3ccn(c3n1)C2 ; FC(F)(F)c1cn(C2CC2)c(-c2ccc3c(c2)OCCCOc2ncnc(C4CC4)c2-c2ncc4ccn(c4n2)C3)n1]

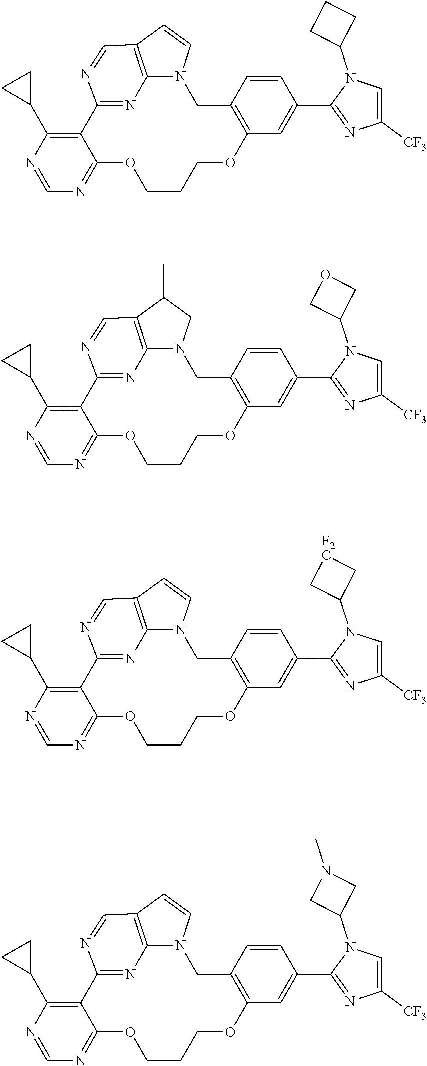 [SMILES: FC(F)(F)c1cn(C2CCC2)c(-c2ccc3c(c2)OCCCOc2ncnc(C4CC4)c2-c2ncc4ccn(c4n2)C3)n1 ; CC1CN2Cc3ccc(-c4nc(C(F)(F)F)cn4C4COC4)cc3OCCCOc3ncnc(C4CC4)c3-c3ncc1c2n3 ; FC1(F)CC(n2cc(C(F)(F)F)nc2-c2ccc3c(c2)OCCCOc2ncnc(C4CC4)c2-c2ncc4ccn(c4n2)C3)C1 ; CN1CC(n2cc(C(F)(F)F)nc2-c2ccc3c(c2)OCCCOc2ncnc(C4CC4)c2-c2ncc4ccn(c4n2)C3)C1]

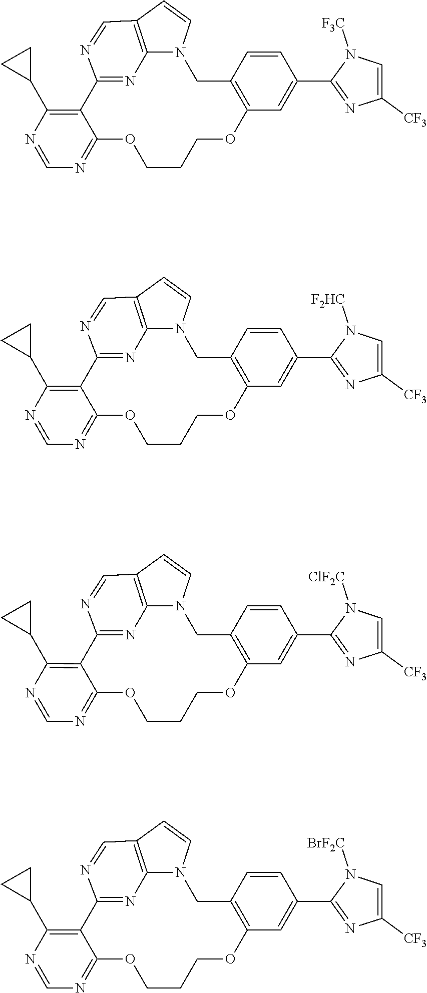

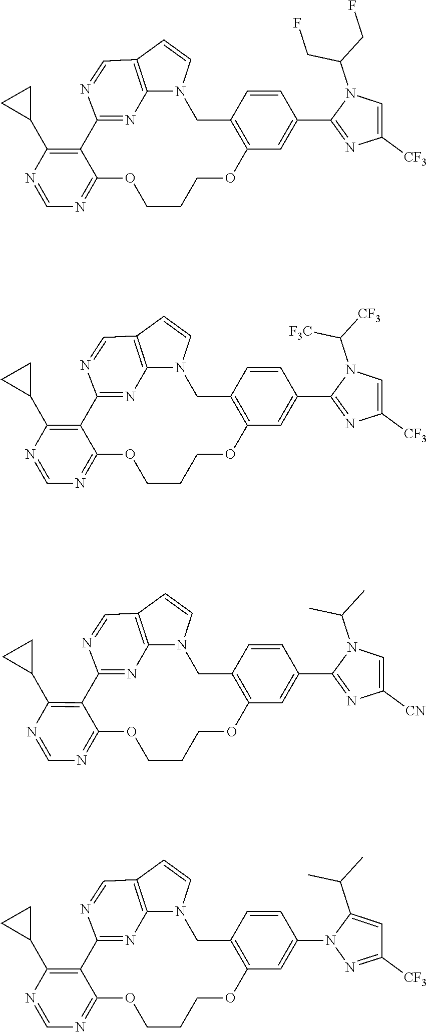 [SMILES: FCC(CF)n1cc(C(F)(F)F)nc1-c1ccc2c(c1)OCCCOc1ncnc(C3CC3)c1-c1ncc3ccn(c3n1)C2 ; FC(F)(F)c1cn(C(C(F)(F)F)C(F)(F)F)c(-c2ccc3c(c2)OCCCOc2ncnc(C4CC4)c2-c2ncc4ccn(c4n2)C3)n1 ; CC(C)n1cc(C#N)nc1-c1ccc2c(c1)OCCCOc1ncnc(C3CC3)c1-c1ncc3ccn(c3n1)C2 ; CC(C)c1cc(C(F)(F)F)nn1-c1ccc2c(c1)OCCCOc1ncnc(C3CC3)c1-c1ncc3ccn(c3n1)C2]

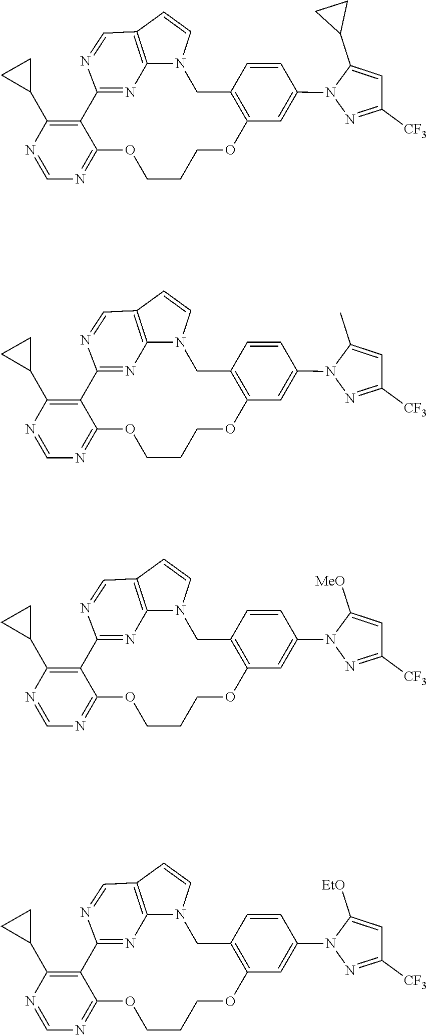 [SMILES: FC(F)(F)c1cc(C2CC2)n(-c2ccc3c(c2)OCCCOc2ncnc(C4CC4)c2-c2ncc4ccn(c4n2)C3)n1 ; Cc1cc(C(F)(F)F)nn1-c1ccc2c(c1)OCCCOc1ncnc(C3CC3)c1-c1ncc3ccn(c3n1)C2 ; COc1cc(C(F)(F)F)nn1-c1ccc2c(c1)OCCCOc1ncnc(C3CC3)c1-c1ncc3ccn(c3n1)C2 ; CCOc1cc(C(F)(F)F)nn1-c1ccc2c(c1)OCCCOc1ncnc(C3CC3)c1-c1ncc3ccn(c3n1)C2]

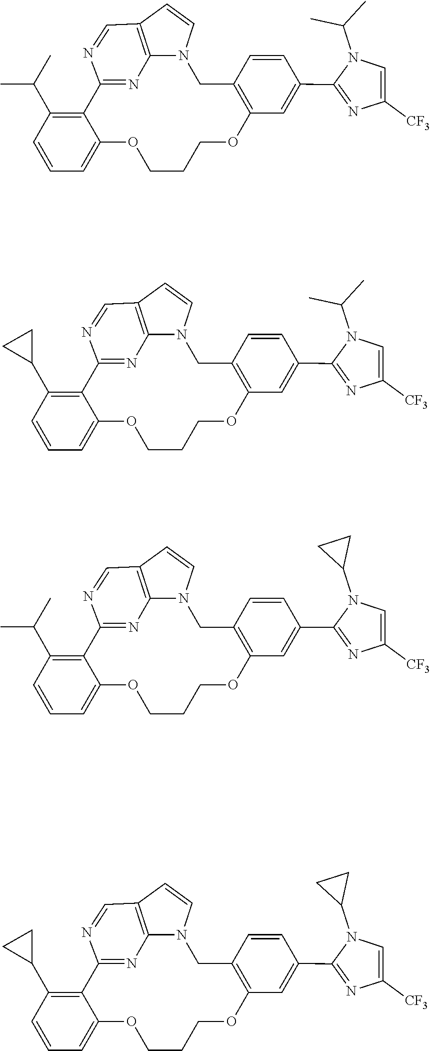 [SMILES: CC(C)c1cccc2c1-c1ncc3ccn(c3n1)Cc1ccc(-c3nc(C(F)(F)F)cn3C(C)C)cc1OCCCO2 ; CC(C)n1cc(C(F)(F)F)nc1-c1ccc2c(c1)OCCCOc1cccc(C3CC3)c1-c1ncc3ccn(c3n1)C2 ; CC(C)c1cccc2c1-c1ncc3ccn(c3n1)Cc1ccc(-c3nc(C(F)(F)F)cn3C3CC3)cc1OCCCO2 ; FC(F)(F)c1cn(C2CC2)c(-c2ccc3c(c2)OCCCOc2cccc(C4CC4)c2-c2ncc4ccn(c4n2)C3)n1]

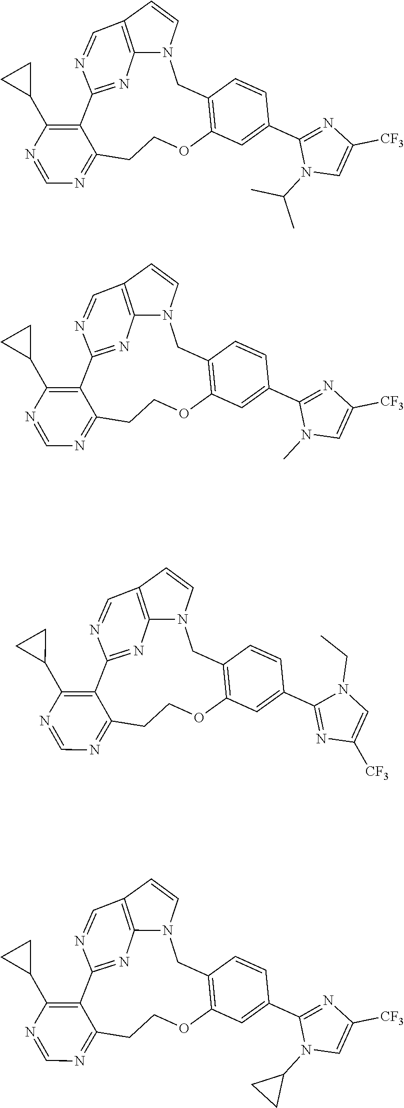 [SMILES: CC(C)n1cc(C(F)(F)F)nc1-c1ccc2c(c1)OCCc1ncnc(C3CC3)c1-c1ncc3ccn(c3n1)C2 ; Cn1cc(C(F)(F)F)nc1-c1ccc2c(c1)OCCc1ncnc(C3CC3)c1-c1ncc3ccn(c3n1)C2 ; CCn1cc(C(F)(F)F)nc1-c1ccc2c(c1)OCCc1ncnc(C3CC3)c1-c1ncc3ccn(c3n1)C2 ; FC(F)(F)c1cn(C2CC2)c(-c2ccc3c(c2)OCCc2ncnc(C4CC4)c2-c2ncc4ccn(c4n2)C3)n1]

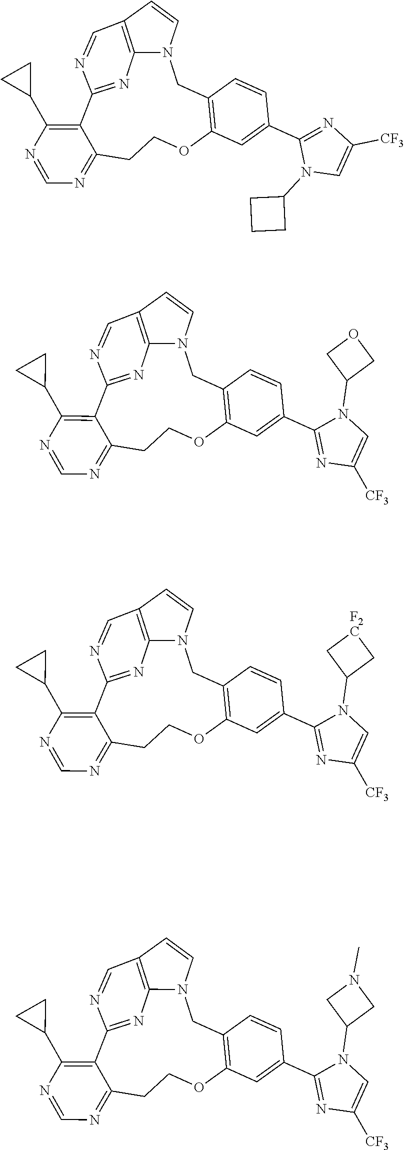 [SMILES: FC(F)(F)c1cn(C2CCC2)c(-c2ccc3c(c2)OCCc2ncnc(C4CC4)c2-c2ncc4ccn(c4n2)C3)n1 ; FC(F)(F)c1cn(C2COC2)c(-c2ccc3c(c2)OCCc2ncnc(C4CC4)c2-c2ncc4ccn(c4n2)C3)n1 ; FC1(F)CC(n2cc(C(F)(F)F)nc2-c2ccc3c(c2)OCCc2ncnc(C4CC4)c2-c2ncc4ccn(c4n2)C3)C1 ; CN1CC(n2cc(C(F)(F)F)nc2-c2ccc3c(c2)OCCc2ncnc(C4CC4)c2-c2ncc4ccn(c4n2)C3)C1]

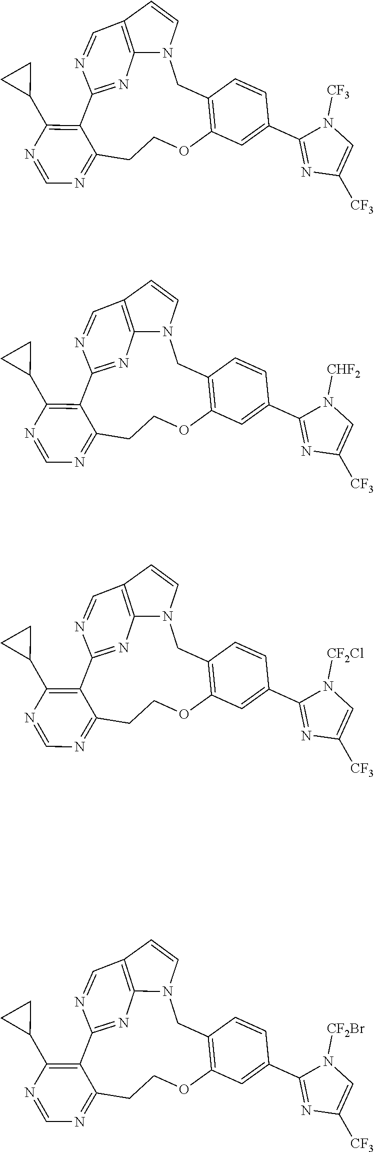

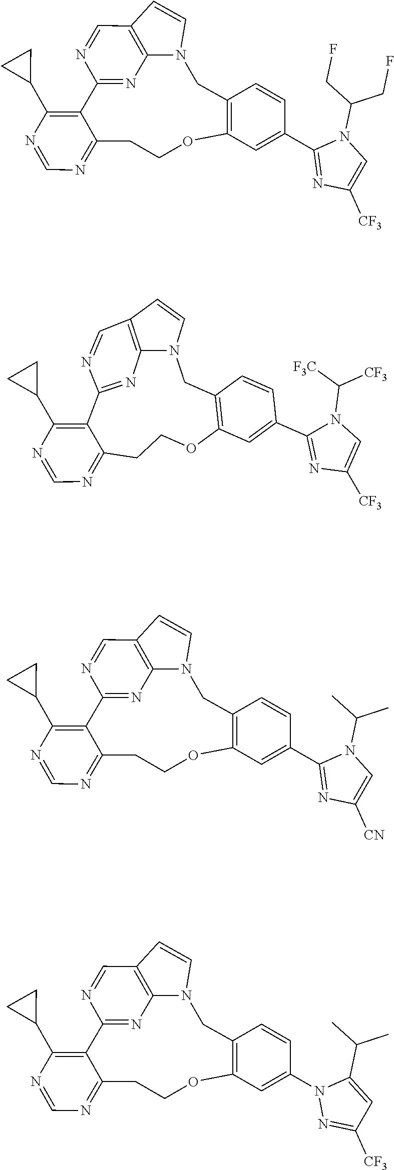 [SMILES: FCC(CF)n1cc(C(F)(F)F)nc1-c1ccc2c(c1)OCCc1ncnc(C3CC3)c1-c1ncc3ccn(c3n1)C2 ; FC(F)(F)c1cn(C(C(F)(F)F)C(F)(F)F)c(-c2ccc3c(c2)OCCc2ncnc(C4CC4)c2-c2ncc4ccn(c4n2)C3)n1 ; CC(C)n1cc(C#N)nc1-c1ccc2c(c1)OCCc1ncnc(C3CC3)c1-c1ncc3ccn(c3n1)C2 ; CC(C)c1cc(C(F)(F)F)nn1-c1ccc2c(c1)OCCc1ncnc(C3CC3)c1-c1ncc3ccn(c3n1)C2]

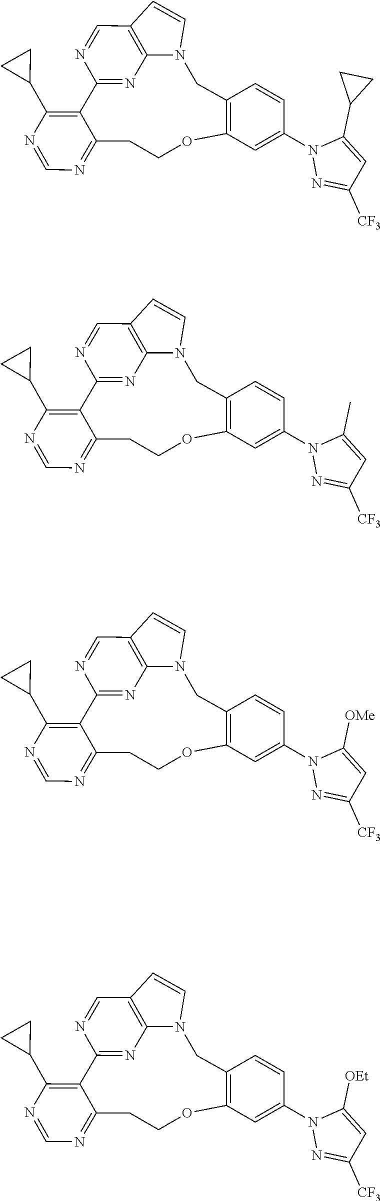 [SMILES: FC(F)(F)c1cc(C2CC2)n(-c2ccc3c(c2)OCCc2ncnc(C4CC4)c2-c2ncc4ccn(c4n2)C3)n1 ; Cc1cc(C(F)(F)F)nn1-c1ccc2c(c1)OCCc1ncnc(C3CC3)c1-c1ncc3ccn(c3n1)C2 ; COc1cc(C(F)(F)F)nn1-c1ccc2c(c1)OCCc1ncnc(C3CC3)c1-c1ncc3ccn(c3n1)C2 ; CCOc1cc(C(F)(F)F)nn1-c1ccc2c(c1)OCCc1ncnc(C3CC3)c1-c1ncc3ccn(c3n1)C2]

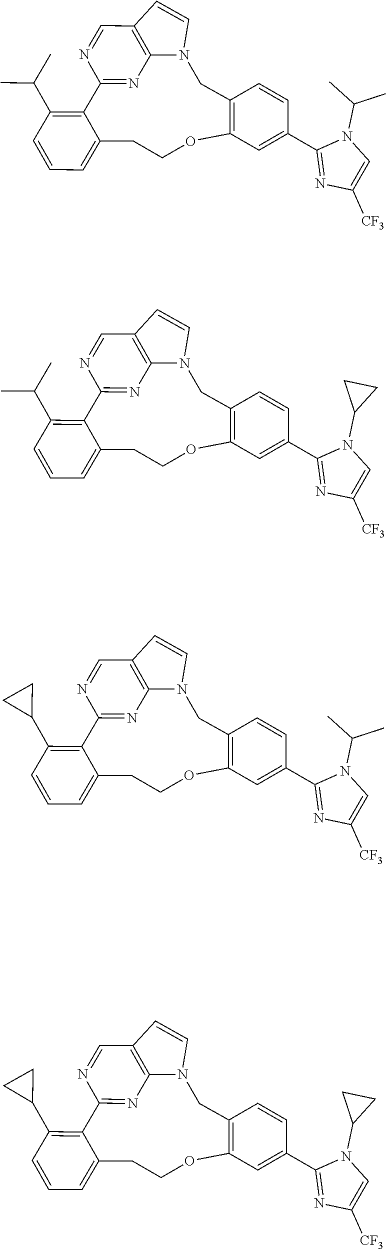

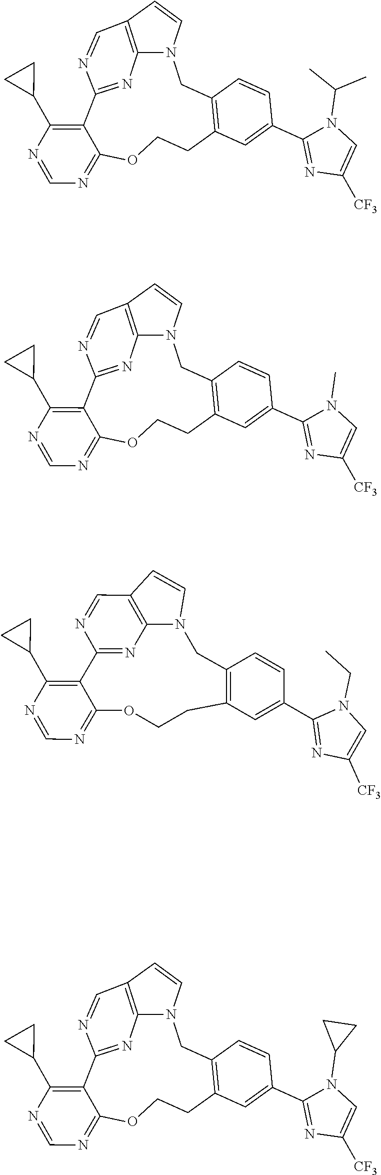 [SMILES: CC(C)n1cc(C(F)(F)F)nc1-c1ccc2c(c1)CCOc1ncnc(C3CC3)c1-c1ncc3ccn(c3n1)C2 ; Cn1cc(C(F)(F)F)nc1-c1ccc2c(c1)CCOc1ncnc(C3CC3)c1-c1ncc3ccn(c3n1)C2 ; CCn1cc(C(F)(F)F)nc1-c1ccc2c(c1)CCOc1ncnc(C3CC3)c1-c1ncc3ccn(c3n1)C2 ; FC(F)(F)c1cn(C2CC2)c(-c2ccc3c(c2)CCOc2ncnc(C4CC4)c2-c2ncc4ccn(c4n2)C3)n1]

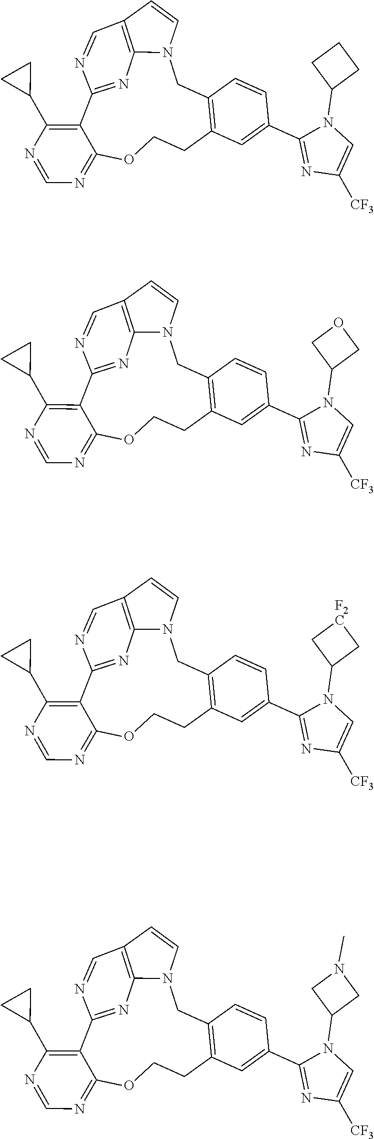 [SMILES: FC(F)(F)c1cn(C2CCC2)c(-c2ccc3c(c2)CCOc2ncnc(C4CC4)c2-c2ncc4ccn(c4n2)C3)n1 ; FC(F)(F)c1cn(C2COC2)c(-c2ccc3c(c2)CCOc2ncnc(C4CC4)c2-c2ncc4ccn(c4n2)C3)n1 ; FC1(F)CC(n2cc(C(F)(F)F)nc2-c2ccc3c(c2)CCOc2ncnc(C4CC4)c2-c2ncc4ccn(c4n2)C3)C1 ; CN1CC(n2cc(C(F)(F)F)nc2-c2ccc3c(c2)CCOc2ncnc(C4CC4)c2-c2ncc4ccn(c4n2)C3)C1]

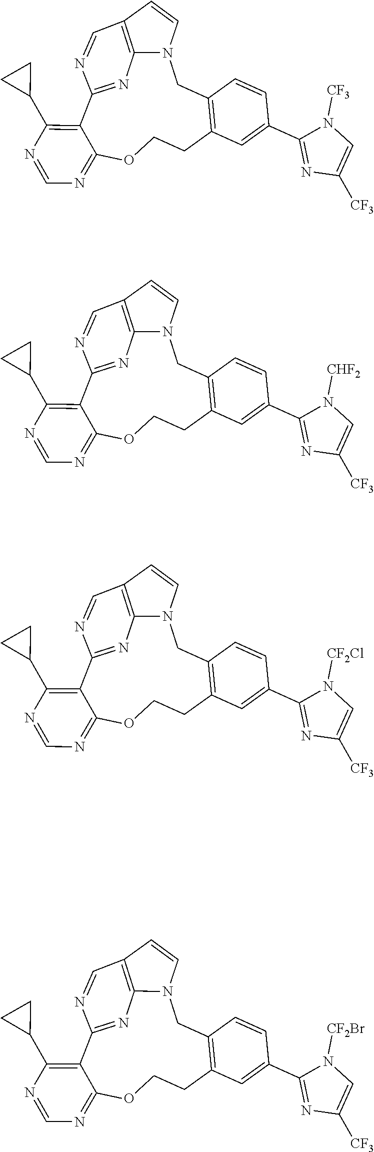

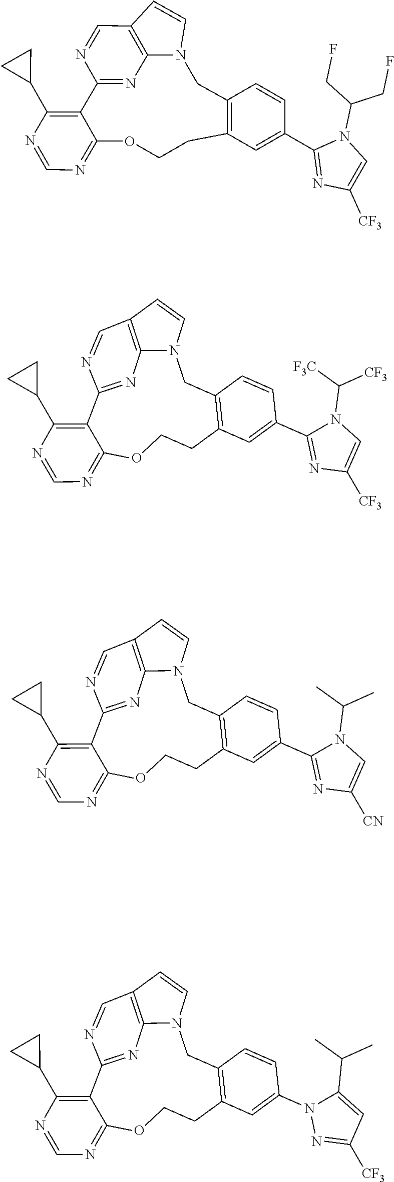 [SMILES: FCC(CF)n1cc(C(F)(F)F)nc1-c1ccc2c(c1)CCOc1ncnc(C3CC3)c1-c1ncc3ccn(c3n1)C2 ; FC(F)(F)c1cn(C(C(F)(F)F)C(F)(F)F)c(-c2ccc3c(c2)CCOc2ncnc(C4CC4)c2-c2ncc4ccn(c4n2)C3)n1 ; CC(C)n1cc(C#N)nc1-c1ccc2c(c1)CCOc1ncnc(C3CC3)c1-c1ncc3ccn(c3n1)C2 ; CC(C)c1cc(C(F)(F)F)nn1-c1ccc2c(c1)CCOc1ncnc(C3CC3)c1-c1ncc3ccn(c3n1)C2]

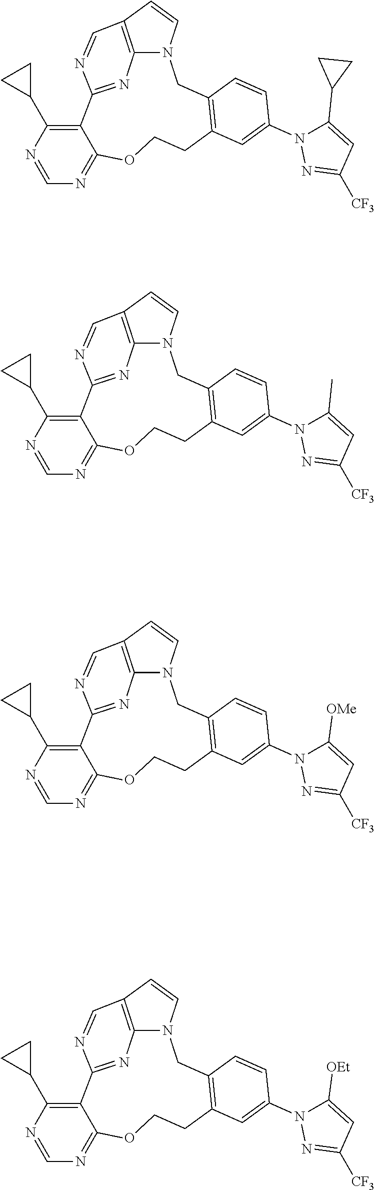 [SMILES: FC(F)(F)c1cc(C2CC2)n(-c2ccc3c(c2)CCOc2ncnc(C4CC4)c2-c2ncc4ccn(c4n2)C3)n1 ; Cc1cc(C(F)(F)F)nn1-c1ccc2c(c1)CCOc1ncnc(C3CC3)c1-c1ncc3ccn(c3n1)C2 ; COc1cc(C(F)(F)F)nn1-c1ccc2c(c1)CCOc1ncnc(C3CC3)c1-c1ncc3ccn(c3n1)C2 ; CCOc1cc(C(F)(F)F)nn1-c1ccc2c(c1)CCOc1ncnc(C3CC3)c1-c1ncc3ccn(c3n1)C2]

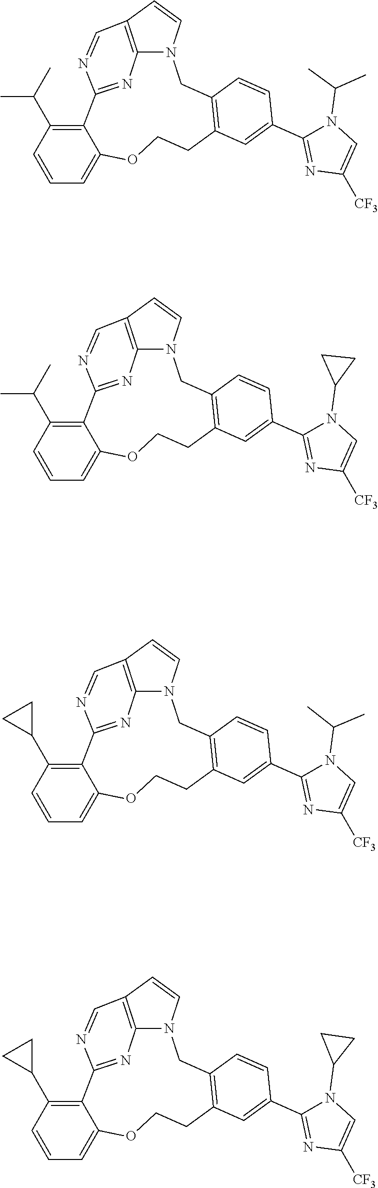

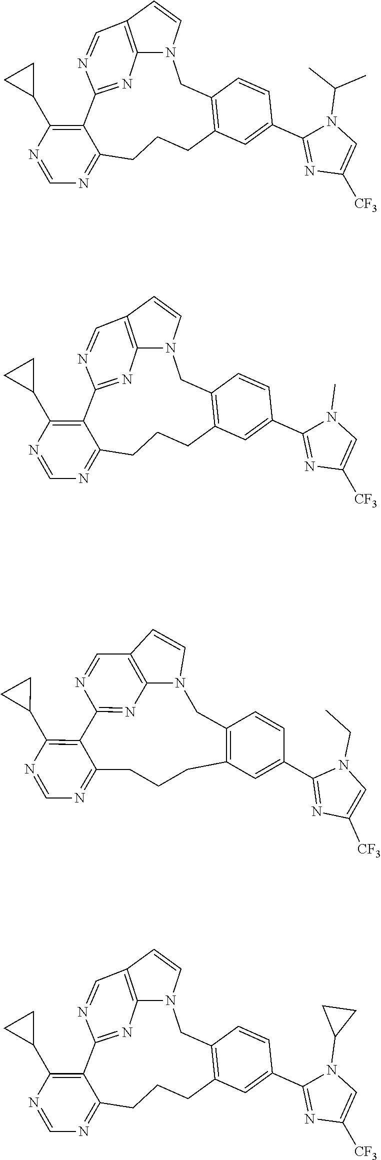 [SMILES: CC(C)n1cc(C(F)(F)F)nc1-c1ccc2c(c1)CCCc1ncnc(C3CC3)c1-c1ncc3ccn(c3n1)C2 ; Cn1cc(C(F)(F)F)nc1-c1ccc2c(c1)CCCc1ncnc(C3CC3)c1-c1ncc3ccn(c3n1)C2 ; CCn1cc(C(F)(F)F)nc1-c1ccc2c(c1)CCCc1ncnc(C3CC3)c1-c1ncc3ccn(c3n1)C2 ; FC(F)(F)c1cn(C2CC2)c(-c2ccc3c(c2)CCCc2ncnc(C4CC4)c2-c2ncc4ccn(c4n2)C3)n1]

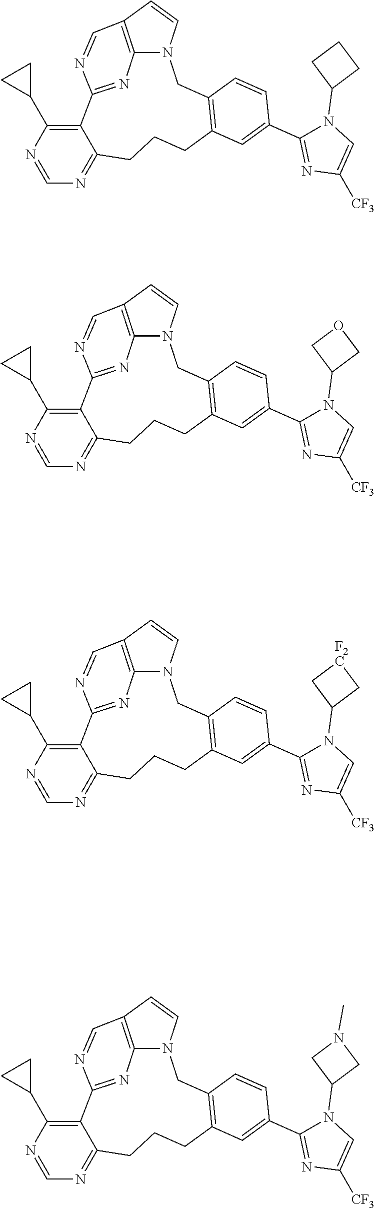 [SMILES: FC(F)(F)c1cn(C2CCC2)c(-c2ccc3c(c2)CCCc2ncnc(C4CC4)c2-c2ncc4ccn(c4n2)C3)n1 ; FC(F)(F)c1cn(C2COC2)c(-c2ccc3c(c2)CCCc2ncnc(C4CC4)c2-c2ncc4ccn(c4n2)C3)n1 ; FC1(F)CC(n2cc(C(F)(F)F)nc2-c2ccc3c(c2)CCCc2ncnc(C4CC4)c2-c2ncc4ccn(c4n2)C3)C1 ; CN1CC(n2cc(C(F)(F)F)nc2-c2ccc3c(c2)CCCc2ncnc(C4CC4)c2-c2ncc4ccn(c4n2)C3)C1]

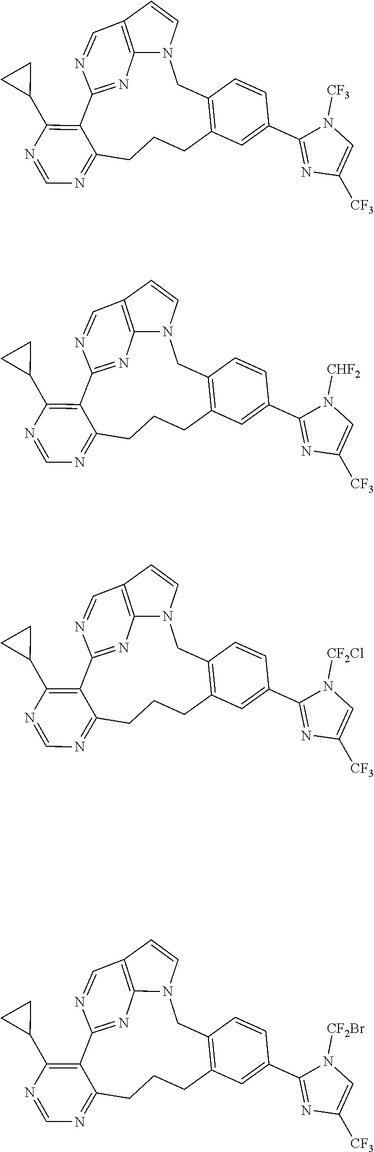

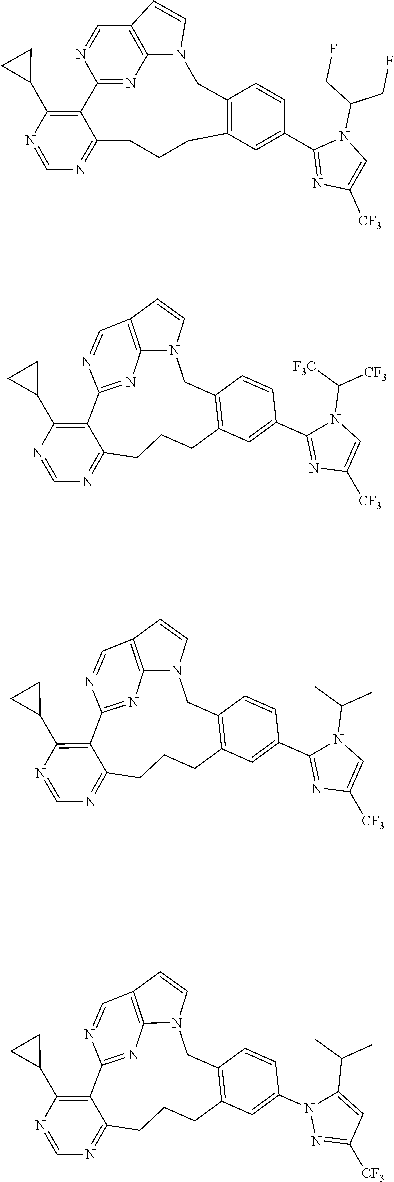 [SMILES: FCC(CF)n1cc(C(F)(F)F)nc1-c1ccc2c(c1)CCCc1ncnc(C3CC3)c1-c1ncc3ccn(c3n1)C2 ; FC(F)(F)c1cn(C(C(F)(F)F)C(F)(F)F)c(-c2ccc3c(c2)CCCc2ncnc(C4CC4)c2-c2ncc4ccn(c4n2)C3)n1 ; CC(C)n1cc(C(F)(F)F)nc1-c1ccc2c(c1)CCCc1ncnc(C3CC3)c1-c1ncc3ccn(c3n1)C2 ; CC(C)c1cc(C(F)(F)F)nn1-c1ccc2c(c1)CCCc1ncnc(C3CC3)c1-c1ncc3ccn(c3n1)C2]

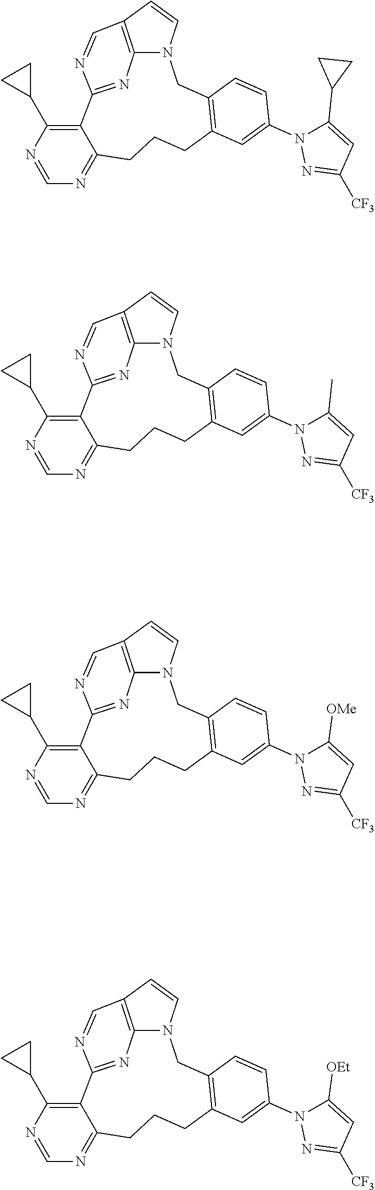 [SMILES: FC(F)(F)c1cc(C2CC2)n(-c2ccc3c(c2)CCCc2ncnc(C4CC4)c2-c2ncc4ccn(c4n2)C3)n1 ; Cc1cc(C(F)(F)F)nn1-c1ccc2c(c1)CCCc1ncnc(C3CC3)c1-c1ncc3ccn(c3n1)C2 ; COc1cc(C(F)(F)F)nn1-c1ccc2c(c1)CCCc1ncnc(C3CC3)c1-c1ncc3ccn(c3n1)C2 ; CCOc1cc(C(F)(F)F)nn1-c1ccc2c(c1)CCCc1ncnc(C3CC3)c1-c1ncc3ccn(c3n1)C2]

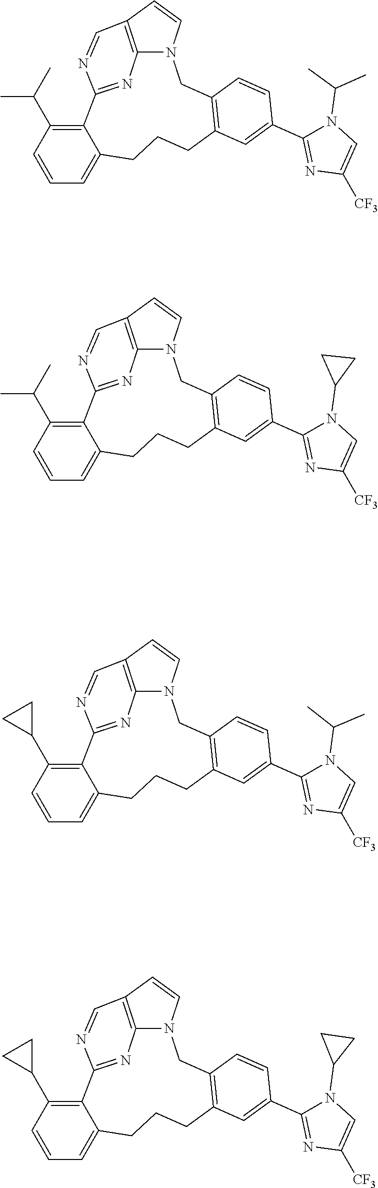

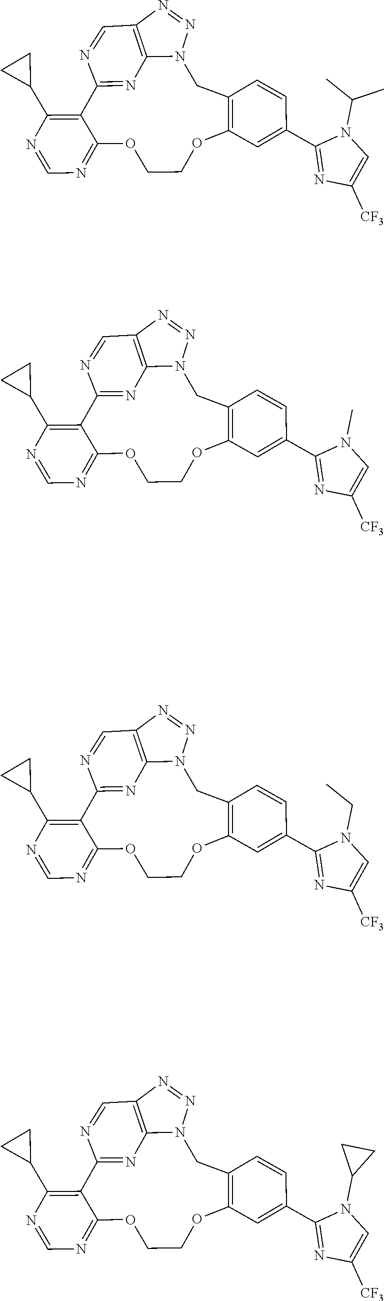 [SMILES: CC(C)n1cc(C(F)(F)F)nc1-c1ccc2c(c1)OCCOc1ncnc(C3CC3)c1-c1ncc3nnn(c3n1)C2 ; Cn1cc(C(F)(F)F)nc1-c1ccc2c(c1)OCCOc1ncnc(C3CC3)c1-c1ncc3nnn(c3n1)C2 ; CCn1cc(C(F)(F)F)nc1-c1ccc2c(c1)OCCOc1ncnc(C3CC3)c1-c1ncc3nnn(c3n1)C2 ; FC(F)(F)c1cn(C2CC2)c(-c2ccc3c(c2)OCCOc2ncnc(C4CC4)c2-c2ncc4nnn(c4n2)C3)n1]

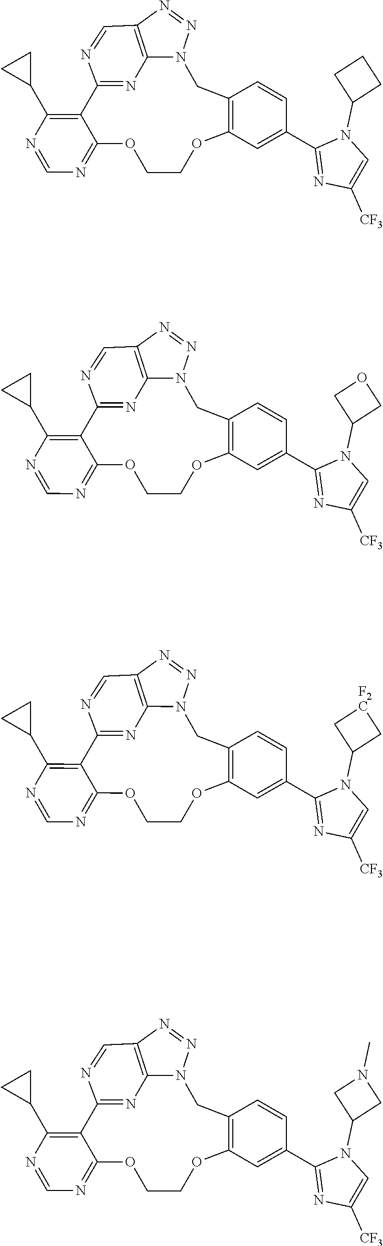 [SMILES: FC(F)(F)c1cn(C2CCC2)c(-c2ccc3c(c2)OCCOc2ncnc(C4CC4)c2-c2ncc4nnn(c4n2)C3)n1 ; FC(F)(F)c1cn(C2COC2)c(-c2ccc3c(c2)OCCOc2ncnc(C4CC4)c2-c2ncc4nnn(c4n2)C3)n1 ; FC1(F)CC(n2cc(C(F)(F)F)nc2-c2ccc3c(c2)OCCOc2ncnc(C4CC4)c2-c2ncc4nnn(c4n2)C3)C1 ; CN1CC(n2cc(C(F)(F)F)nc2-c2ccc3c(c2)OCCOc2ncnc(C4CC4)c2-c2ncc4nnn(c4n2)C3)C1]

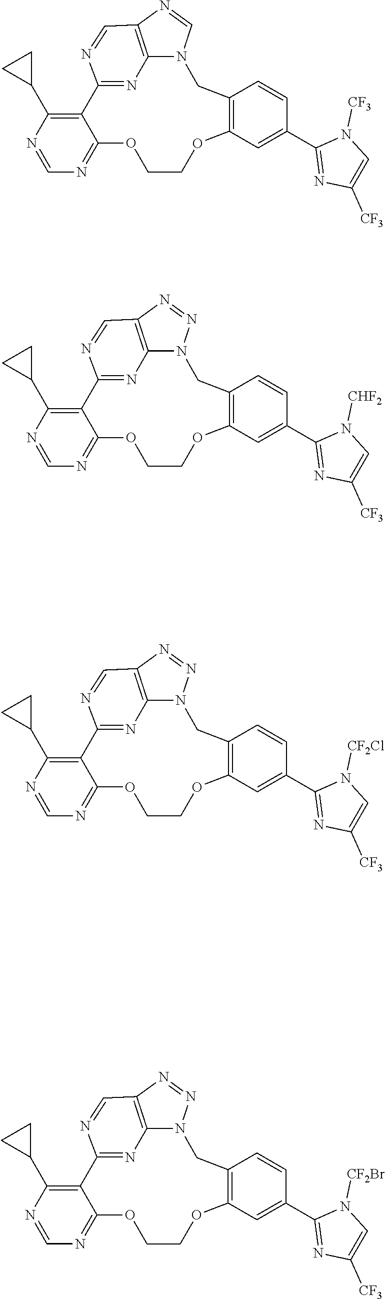

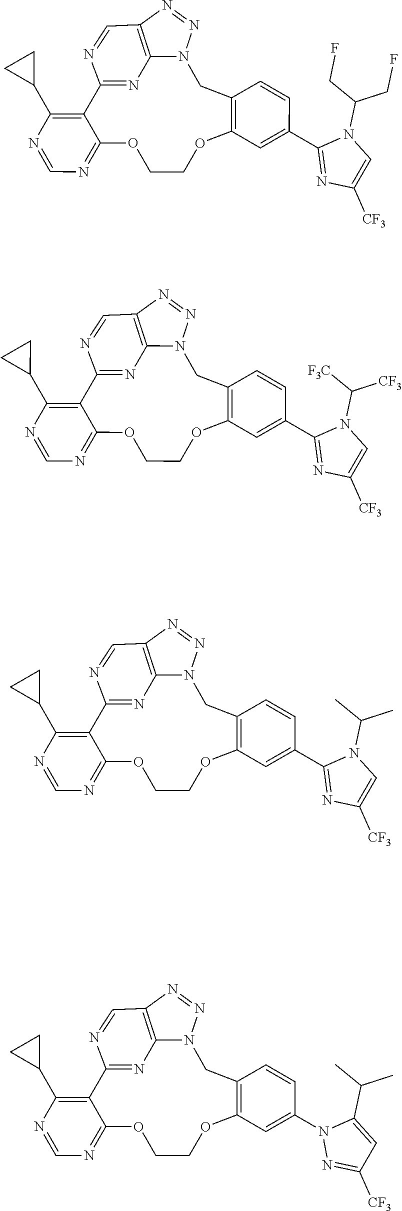 [SMILES: FCC(CF)n1cc(C(F)(F)F)nc1-c1ccc2c(c1)OCCOc1ncnc(C3CC3)c1-c1ncc3nnn(c3n1)C2 ; FC(F)(F)c1cn(C(C(F)(F)F)C(F)(F)F)c(-c2ccc3c(c2)OCCOc2ncnc(C4CC4)c2-c2ncc4nnn(c4n2)C3)n1 ; CC(C)n1cc(C(F)(F)F)nc1-c1ccc2c(c1)OCCOc1ncnc(C3CC3)c1-c1ncc3nnn(c3n1)C2 ; CC(C)c1cc(C(F)(F)F)nn1-c1ccc2c(c1)OCCOc1ncnc(C3CC3)c1-c1ncc3nnn(c3n1)C2]

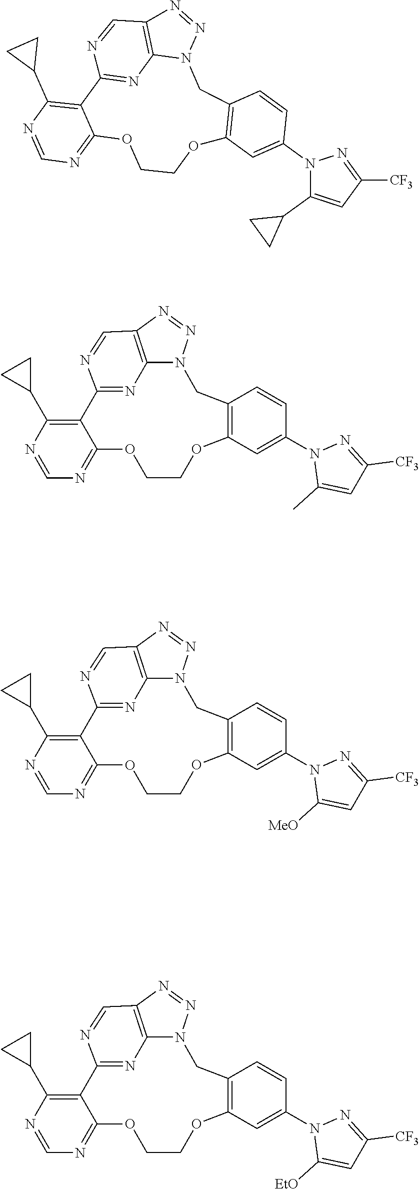 [SMILES: FC(F)(F)c1cc(C2CC2)n(-c2ccc3c(c2)OCCOc2ncnc(C4CC4)c2-c2ncc4nnn(c4n2)C3)n1 ; Cc1cc(C(F)(F)F)nn1-c1ccc2c(c1)OCCOc1ncnc(C3CC3)c1-c1ncc3nnn(c3n1)C2 ; COc1cc(C(F)(F)F)nn1-c1ccc2c(c1)OCCOc1ncnc(C3CC3)c1-c1ncc3nnn(c3n1)C2 ; CCOc1cc(C(F)(F)F)nn1-c1ccc2c(c1)OCCOc1ncnc(C3CC3)c1-c1ncc3nnn(c3n1)C2]

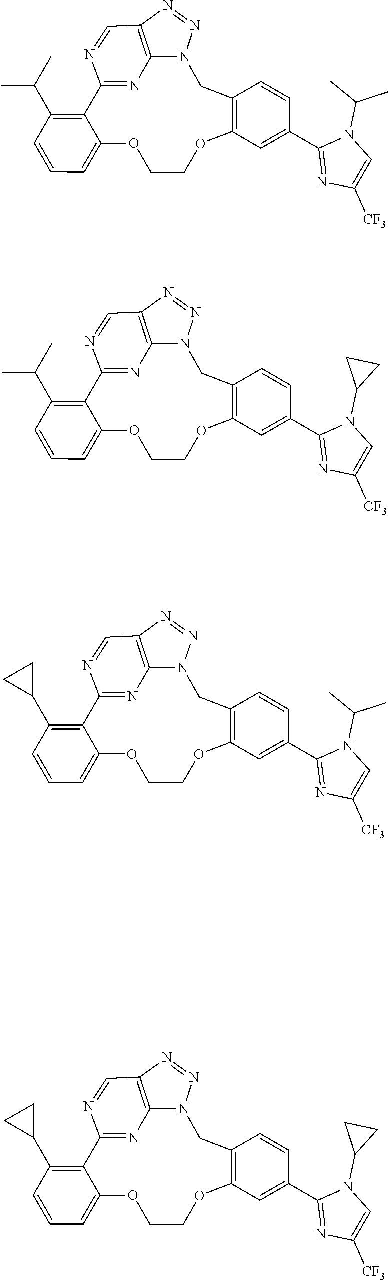

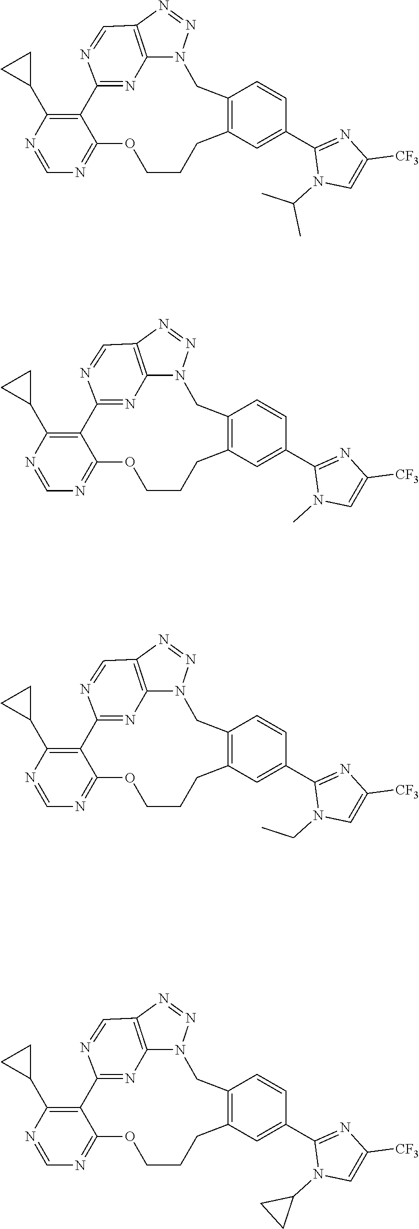 [SMILES: CC(C)n1cc(C(F)(F)F)nc1-c1ccc2c(c1)CCCOc1ncnc(C3CC3)c1-c1ncc3nnn(c3n1)C2 ; Cn1cc(C(F)(F)F)nc1-c1ccc2c(c1)CCCOc1ncnc(C3CC3)c1-c1ncc3nnn(c3n1)C2 ; CCn1cc(C(F)(F)F)nc1-c1ccc2c(c1)CCCOc1ncnc(C3CC3)c1-c1ncc3nnn(c3n1)C2 ; FC(F)(F)c1cn(C2CC2)c(-c2ccc3c(c2)CCCOc2ncnc(C4CC4)c2-c2ncc4nnn(c4n2)C3)n1]

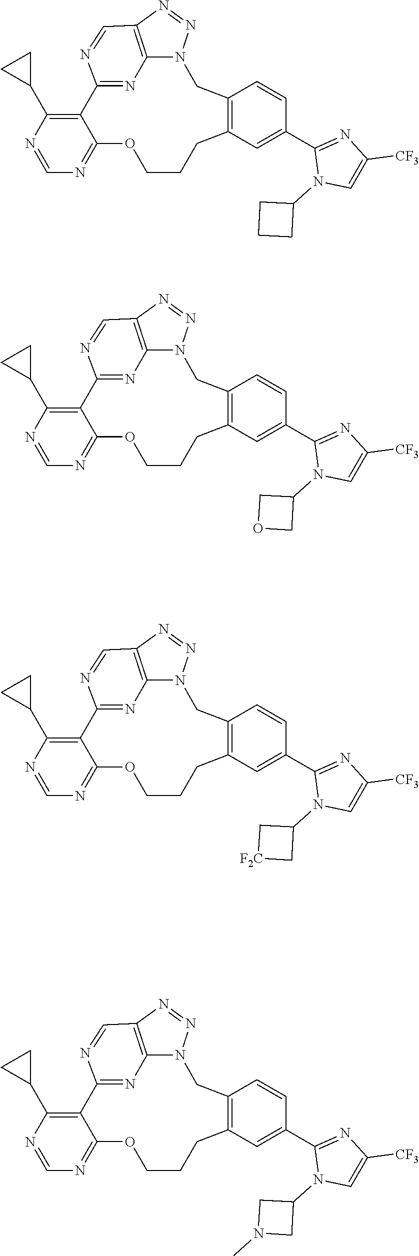 [SMILES: FC(F)(F)c1cn(C2CCC2)c(-c2ccc3c(c2)CCCOc2ncnc(C4CC4)c2-c2ncc4nnn(c4n2)C3)n1 ; FC(F)(F)c1cn(C2COC2)c(-c2ccc3c(c2)CCCOc2ncnc(C4CC4)c2-c2ncc4nnn(c4n2)C3)n1 ; FC1(F)CC(n2cc(C(F)(F)F)nc2-c2ccc3c(c2)CCCOc2ncnc(C4CC4)c2-c2ncc4nnn(c4n2)C3)C1 ; CN1CC(n2cc(C(F)(F)F)nc2-c2ccc3c(c2)CCCOc2ncnc(C4CC4)c2-c2ncc4nnn(c4n2)C3)C1]

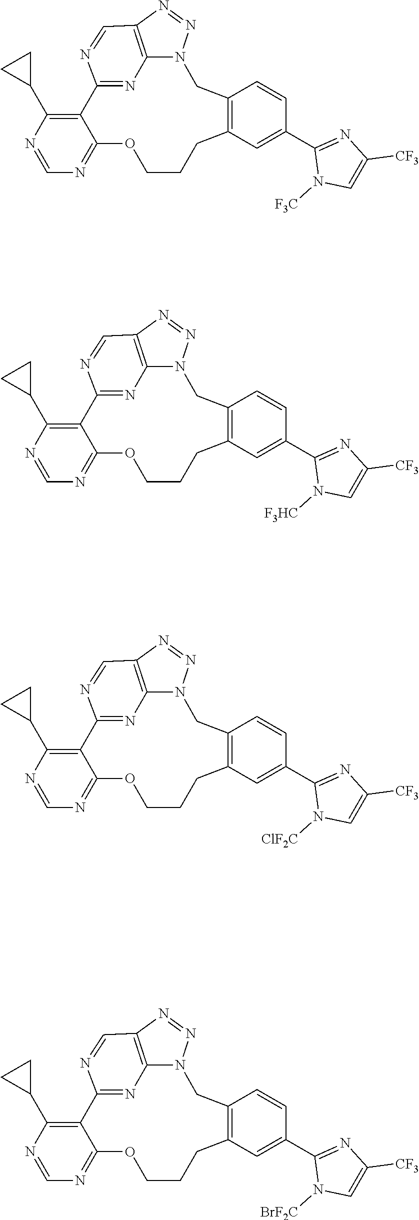

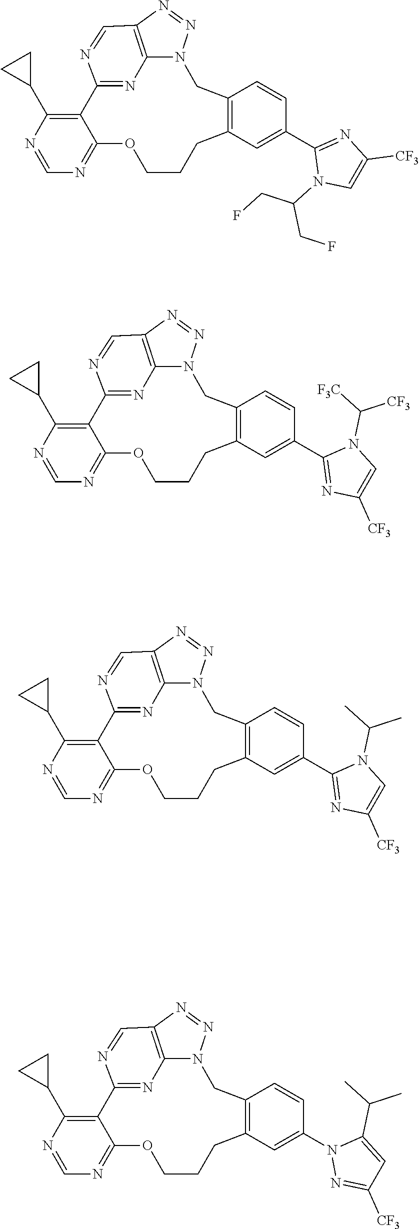 [SMILES: FCC(CF)n1cc(C(F)(F)F)nc1-c1ccc2c(c1)CCCOc1ncnc(C3CC3)c1-c1ncc3nnn(c3n1)C2 ; FC(F)(F)c1cn(C(C(F)(F)F)C(F)(F)F)c(-c2ccc3c(c2)CCCOc2ncnc(C4CC4)c2-c2ncc4nnn(c4n2)C3)n1 ; CC(C)n1cc(C(F)(F)F)nc1-c1ccc2c(c1)CCCOc1ncnc(C3CC3)c1-c1ncc3nnn(c3n1)C2 ; CC(C)c1cc(C(F)(F)F)nn1-c1ccc2c(c1)CCCOc1ncnc(C3CC3)c1-c1ncc3nnn(c3n1)C2]

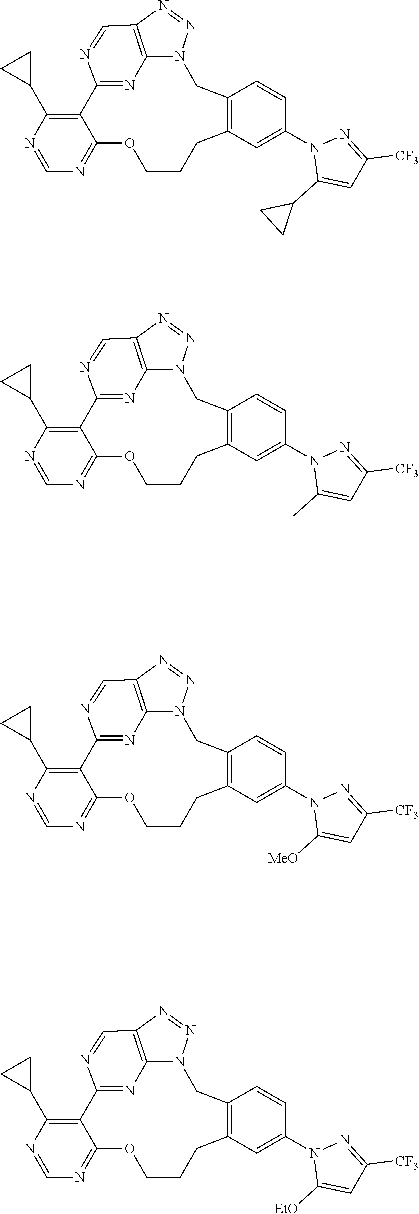 [SMILES: FC(F)(F)c1cc(C2CC2)n(-c2ccc3c(c2)CCCOc2ncnc(C4CC4)c2-c2ncc4nnn(c4n2)C3)n1 ; Cc1cc(C(F)(F)F)nn1-c1ccc2c(c1)CCCOc1ncnc(C3CC3)c1-c1ncc3nnn(c3n1)C2 ; COc1cc(C(F)(F)F)nn1-c1ccc2c(c1)CCCOc1ncnc(C3CC3)c1-c1ncc3nnn(c3n1)C2 ; CCOc1cc(C(F)(F)F)nn1-c1ccc2c(c1)CCCOc1ncnc(C3CC3)c1-c1ncc3nnn(c3n1)C2]

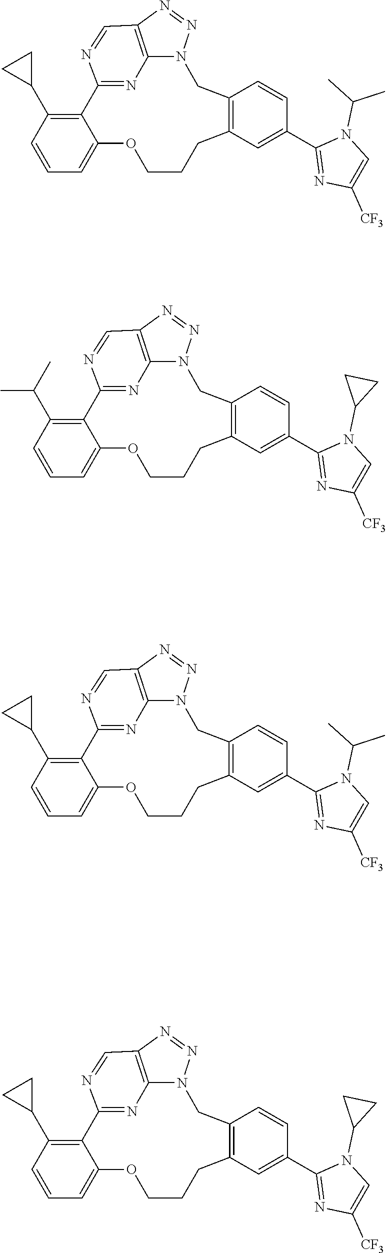

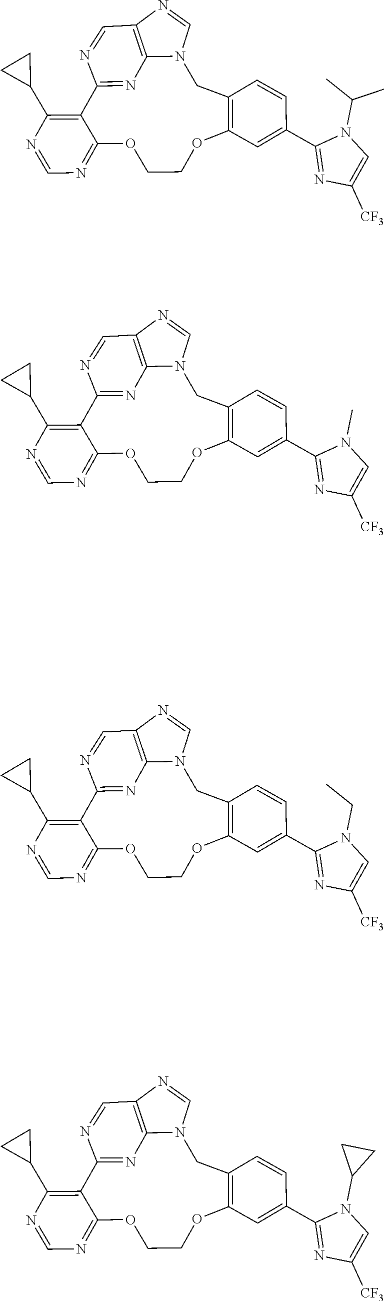 [SMILES: CC(C)n1cc(C(F)(F)F)nc1-c1ccc2c(c1)OCCOc1ncnc(C3CC3)c1-c1ncc3ncn(c3n1)C2 ; Cn1cc(C(F)(F)F)nc1-c1ccc2c(c1)OCCOc1ncnc(C3CC3)c1-c1ncc3ncn(c3n1)C2 ; CCn1cc(C(F)(F)F)nc1-c1ccc2c(c1)OCCOc1ncnc(C3CC3)c1-c1ncc3ncn(c3n1)C2 ; FC(F)(F)c1cn(C2CC2)c(-c2ccc3c(c2)OCCOc2ncnc(C4CC4)c2-c2ncc4ncn(c4n2)C3)n1]

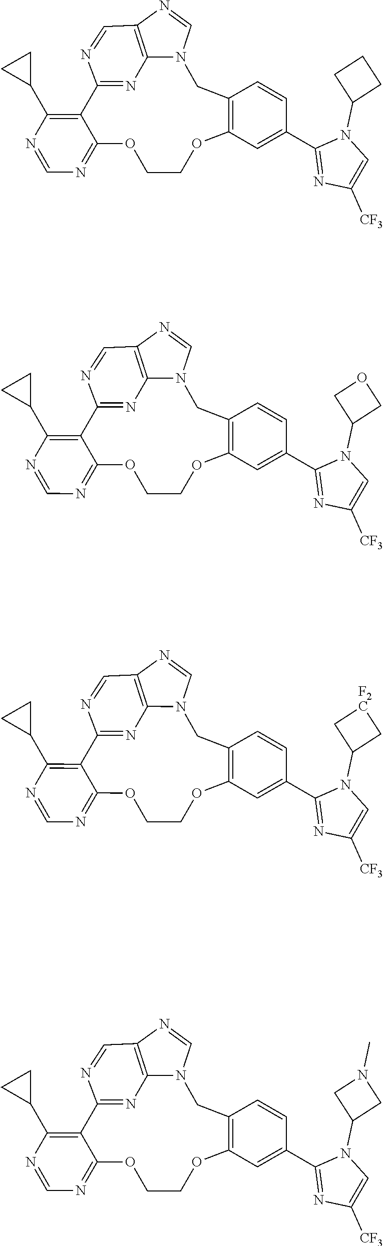 [SMILES: FC(F)(F)c1cn(C2CCC2)c(-c2ccc3c(c2)OCCOc2ncnc(C4CC4)c2-c2ncc4ncn(c4n2)C3)n1 ; FC(F)(F)c1cn(C2COC2)c(-c2ccc3c(c2)OCCOc2ncnc(C4CC4)c2-c2ncc4ncn(c4n2)C3)n1 ; FC1(F)CC(n2cc(C(F)(F)F)nc2-c2ccc3c(c2)OCCOc2ncnc(C4CC4)c2-c2ncc4ncn(c4n2)C3)C1 ; CN1CC(n2cc(C(F)(F)F)nc2-c2ccc3c(c2)OCCOc2ncnc(C4CC4)c2-c2ncc4ncn(c4n2)C3)C1]

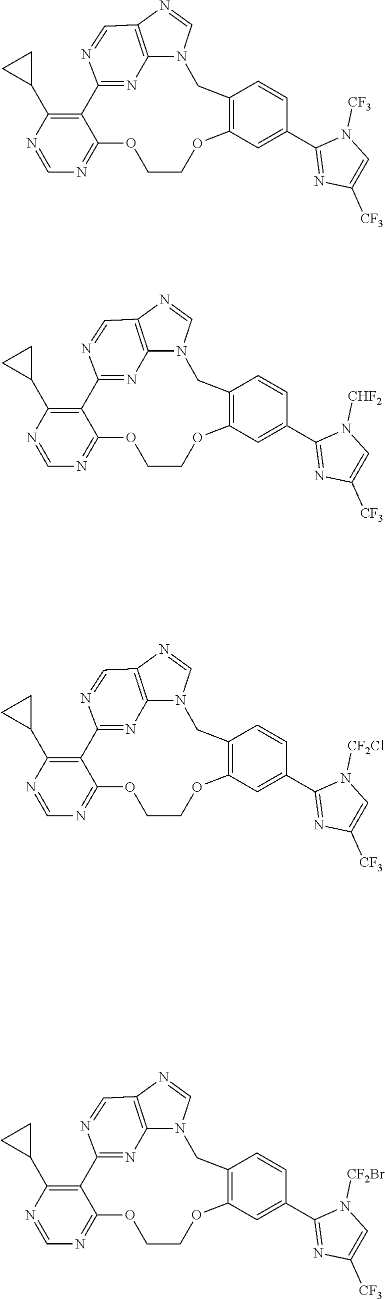

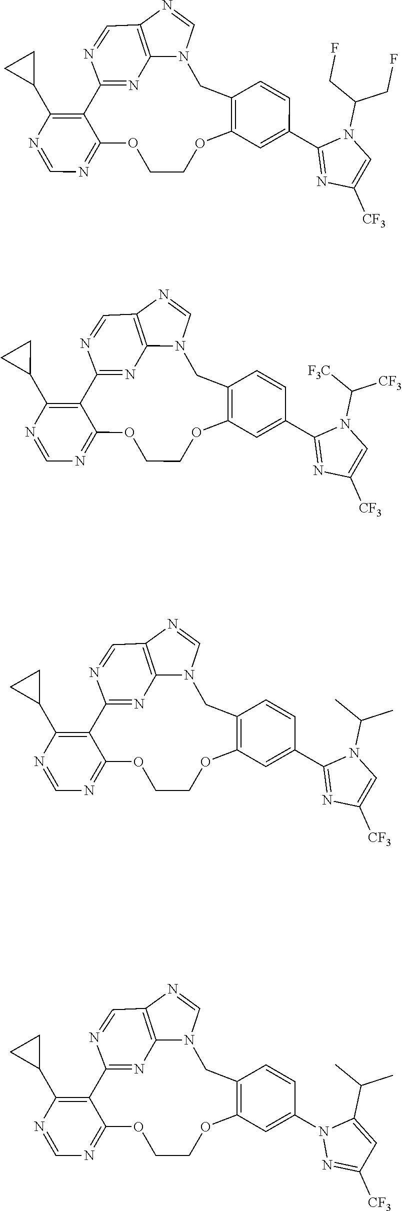 [SMILES: FCC(CF)n1cc(C(F)(F)F)nc1-c1ccc2c(c1)OCCOc1ncnc(C3CC3)c1-c1ncc3ncn(c3n1)C2 ; FC(F)(F)c1cn(C(C(F)(F)F)C(F)(F)F)c(-c2ccc3c(c2)OCCOc2ncnc(C4CC4)c2-c2ncc4ncn(c4n2)C3)n1 ; CC(C)n1cc(C(F)(F)F)nc1-c1ccc2c(c1)OCCOc1ncnc(C3CC3)c1-c1ncc3ncn(c3n1)C2 ; CC(C)c1cc(C(F)(F)F)nn1-c1ccc2c(c1)OCCOc1ncnc(C3CC3)c1-c1ncc3ncn(c3n1)C2]

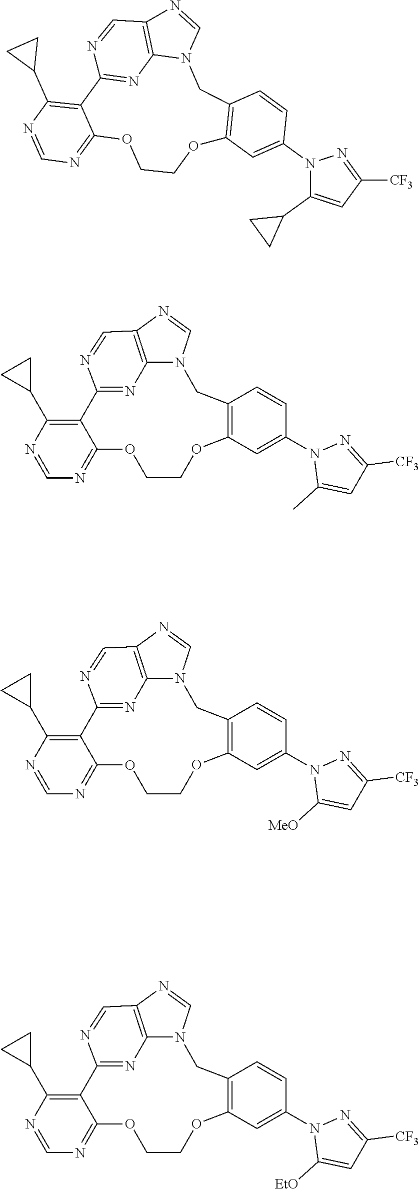 [SMILES: FC(F)(F)c1cc(C2CC2)n(-c2ccc3c(c2)OCCOc2ncnc(C4CC4)c2-c2ncc4ncn(c4n2)C3)n1 ; Cc1cc(C(F)(F)F)nn1-c1ccc2c(c1)OCCOc1ncnc(C3CC3)c1-c1ncc3ncn(c3n1)C2 ; COc1cc(C(F)(F)F)nn1-c1ccc2c(c1)OCCOc1ncnc(C3CC3)c1-c1ncc3ncn(c3n1)C2 ; CCOc1cc(C(F)(F)F)nn1-c1ccc2c(c1)OCCOc1ncnc(C3CC3)c1-c1ncc3ncn(c3n1)C2]

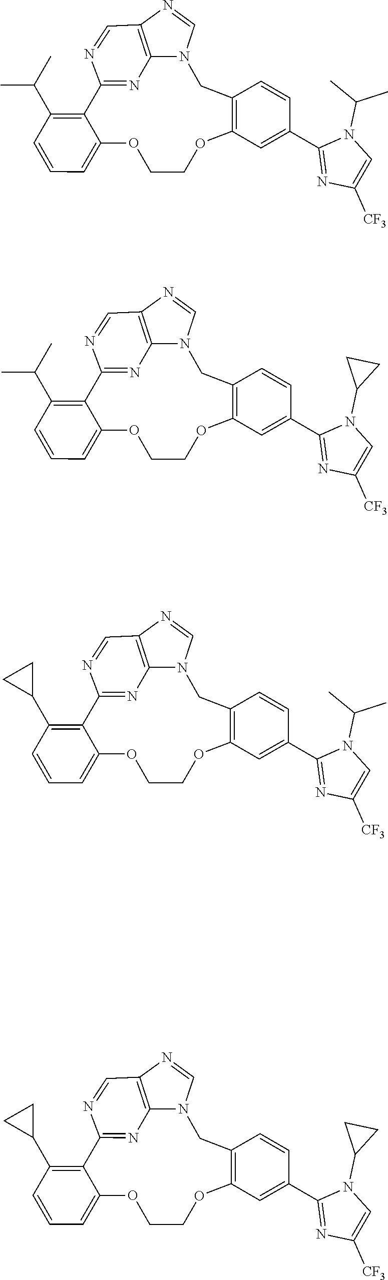

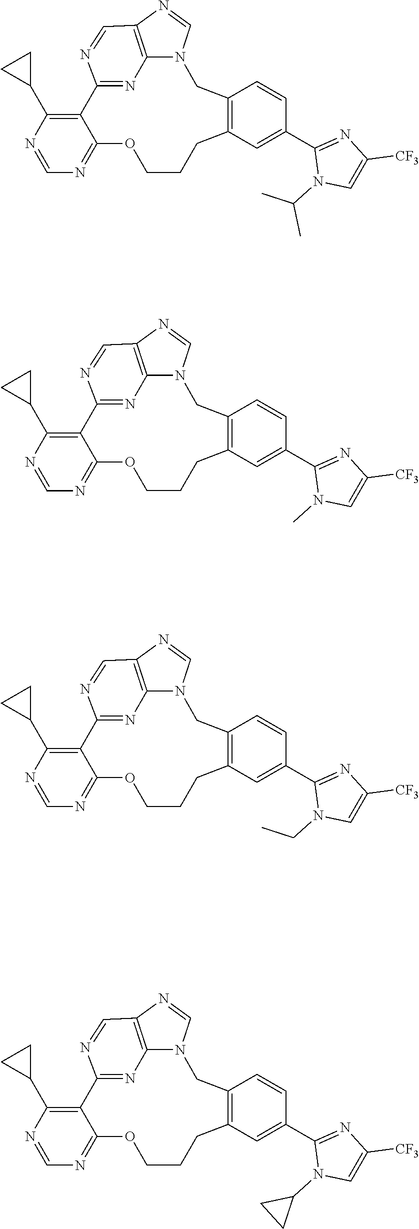 [SMILES: CC(C)n1cc(C(F)(F)F)nc1-c1ccc2c(c1)CCCOc1ncnc(C3CC3)c1-c1ncc3ncn(c3n1)C2 ; Cn1cc(C(F)(F)F)nc1-c1ccc2c(c1)CCCOc1ncnc(C3CC3)c1-c1ncc3ncn(c3n1)C2 ; CCn1cc(C(F)(F)F)nc1-c1ccc2c(c1)CCCOc1ncnc(C3CC3)c1-c1ncc3ncn(c3n1)C2 ; FC(F)(F)c1cn(C2CC2)c(-c2ccc3c(c2)CCCOc2ncnc(C4CC4)c2-c2ncc4ncn(c4n2)C3)n1]

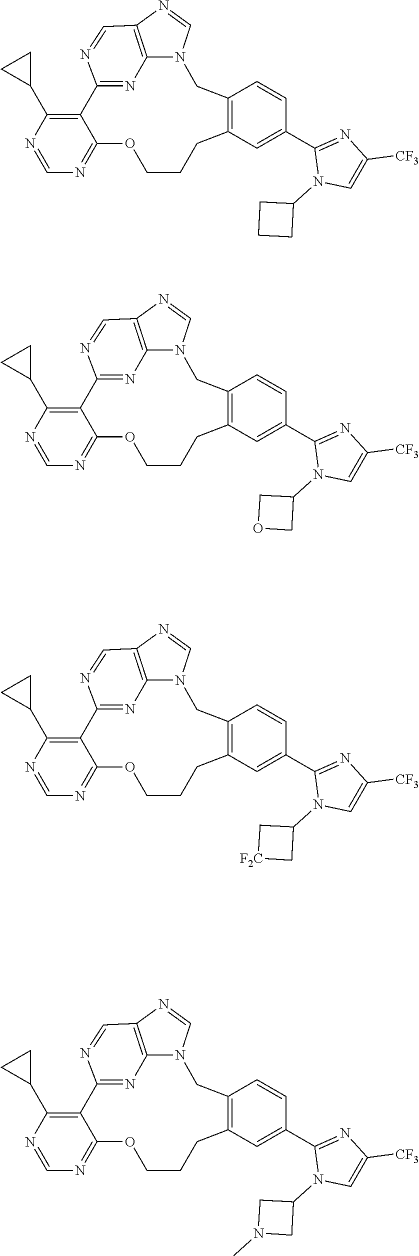 [SMILES: FC(F)(F)c1cn(C2CCC2)c(-c2ccc3c(c2)CCCOc2ncnc(C4CC4)c2-c2ncc4ncn(c4n2)C3)n1 ; FC(F)(F)c1cn(C2COC2)c(-c2ccc3c(c2)CCCOc2ncnc(C4CC4)c2-c2ncc4ncn(c4n2)C3)n1 ; FC1(F)CC(n2cc(C(F)(F)F)nc2-c2ccc3c(c2)CCCOc2ncnc(C4CC4)c2-c2ncc4ncn(c4n2)C3)C1 ; CN1CC(n2cc(C(F)(F)F)nc2-c2ccc3c(c2)CCCOc2ncnc(C4CC4)c2-c2ncc4ncn(c4n2)C3)C1]

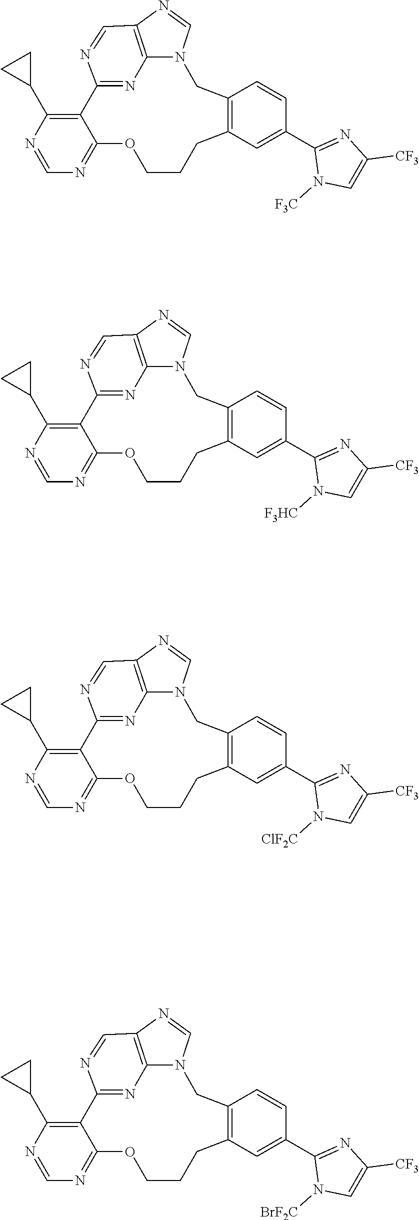

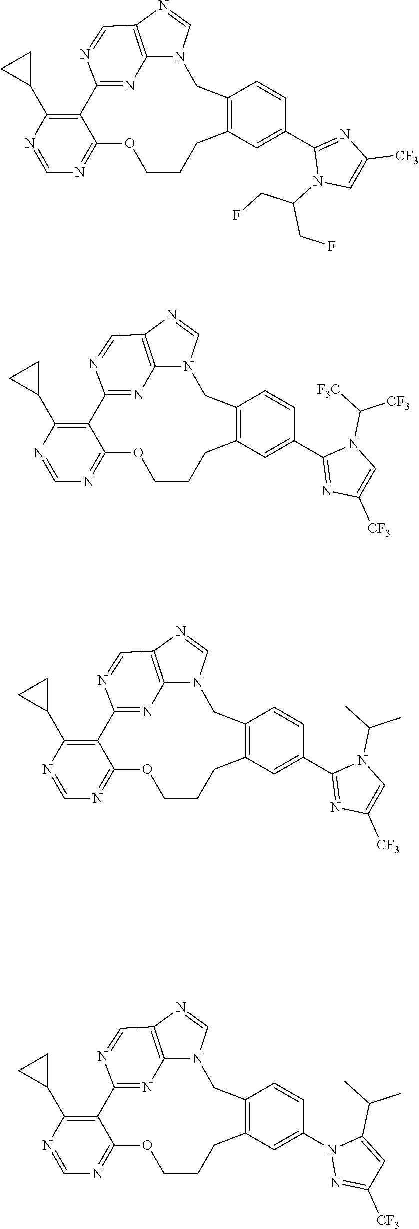 [SMILES: FCC(CF)n1cc(C(F)(F)F)nc1-c1ccc2c(c1)CCCOc1ncnc(C3CC3)c1-c1ncc3ncn(c3n1)C2 ; FC(F)(F)c1cn(C(C(F)(F)F)C(F)(F)F)c(-c2ccc3c(c2)CCCOc2ncnc(C4CC4)c2-c2ncc4ncn(c4n2)C3)n1 ; CC(C)n1cc(C(F)(F)F)nc1-c1ccc2c(c1)CCCOc1ncnc(C3CC3)c1-c1ncc3ncn(c3n1)C2 ; CC(C)c1cc(C(F)(F)F)nn1-c1ccc2c(c1)CCCOc1ncnc(C3CC3)c1-c1ncc3ncn(c3n1)C2]

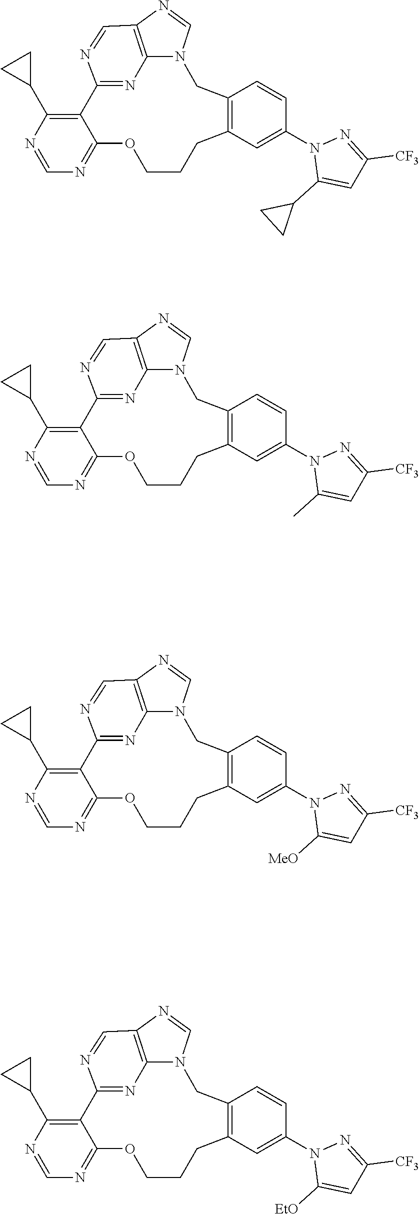 [SMILES: FC(F)(F)c1cc(C2CC2)n(-c2ccc3c(c2)CCCOc2ncnc(C4CC4)c2-c2ncc4ncn(c4n2)C3)n1 ; Cc1cc(C(F)(F)F)nn1-c1ccc2c(c1)CCCOc1ncnc(C3CC3)c1-c1ncc3ncn(c3n1)C2 ; COc1cc(C(F)(F)F)nn1-c1ccc2c(c1)CCCOc1ncnc(C3CC3)c1-c1ncc3ncn(c3n1)C2 ; CCOc1cc(C(F)(F)F)nn1-c1ccc2c(c1)CCCOc1ncnc(C3CC3)c1-c1ncc3ncn(c3n1)C2]

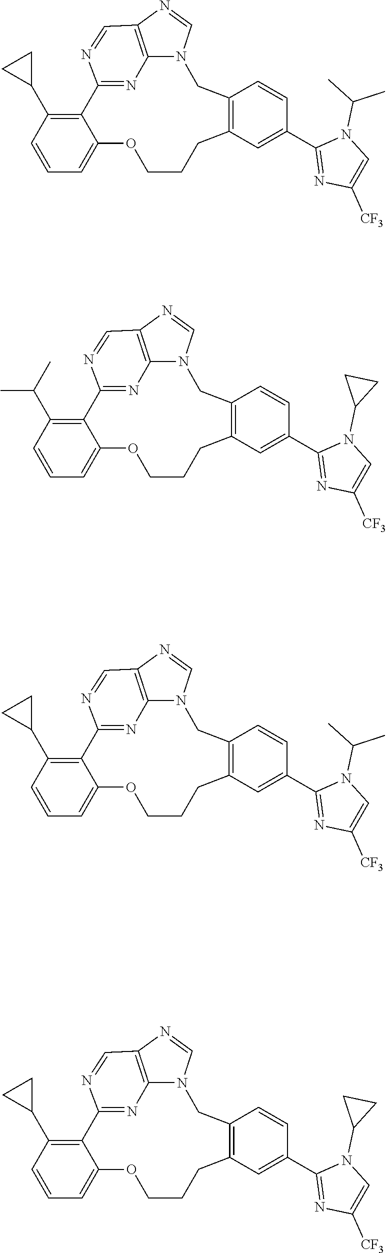

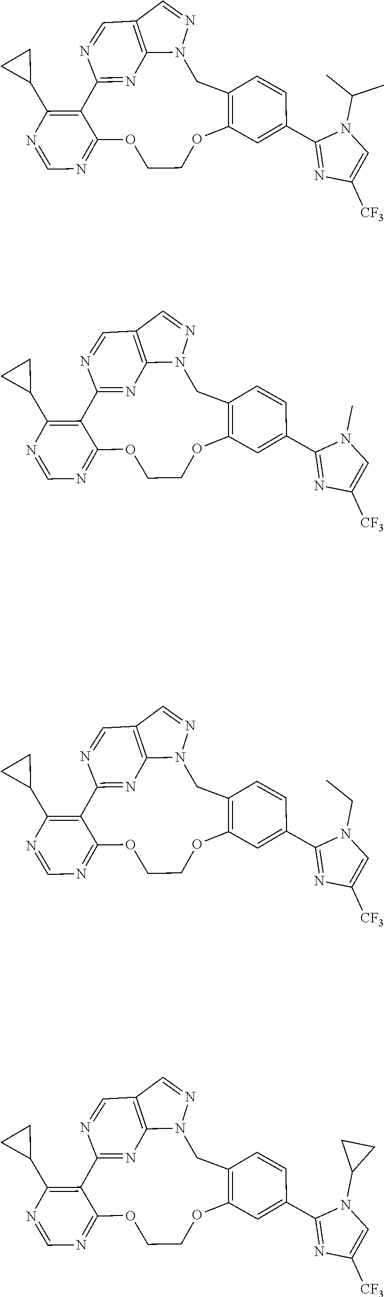 [SMILES: CC(C)n1cc(C(F)(F)F)nc1-c1ccc2c(c1)OCCOc1ncnc(C3CC3)c1-c1ncc3cnn(c3n1)C2 ; Cn1cc(C(F)(F)F)nc1-c1ccc2c(c1)OCCOc1ncnc(C3CC3)c1-c1ncc3cnn(c3n1)C2 ; CCn1cc(C(F)(F)F)nc1-c1ccc2c(c1)OCCOc1ncnc(C3CC3)c1-c1ncc3cnn(c3n1)C2 ; FC(F)(F)c1cn(C2CC2)c(-c2ccc3c(c2)OCCOc2ncnc(C4CC4)c2-c2ncc4cnn(c4n2)C3)n1]

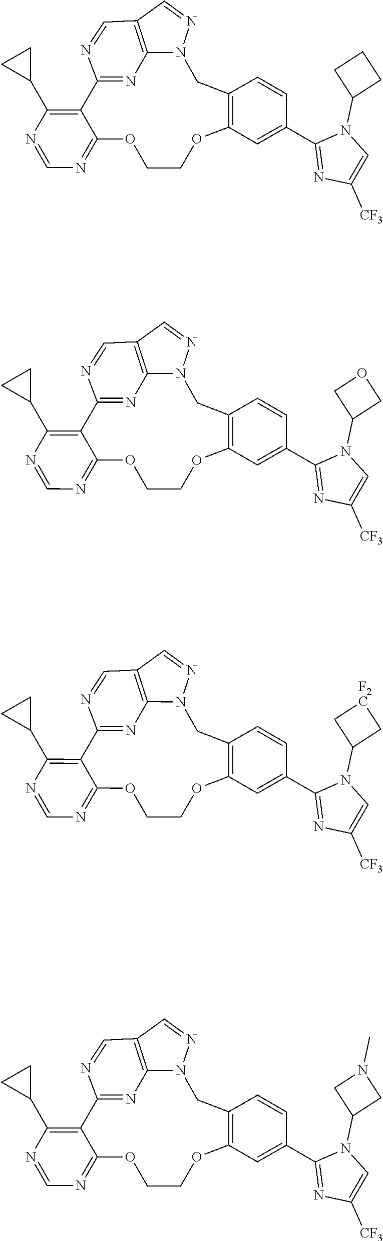 [SMILES: FC(F)(F)c1cn(C2CCC2)c(-c2ccc3c(c2)OCCOc2ncnc(C4CC4)c2-c2ncc4cnn(c4n2)C3)n1 ; FC(F)(F)c1cn(C2COC2)c(-c2ccc3c(c2)OCCOc2ncnc(C4CC4)c2-c2ncc4cnn(c4n2)C3)n1 ; FC1(F)CC(n2cc(C(F)(F)F)nc2-c2ccc3c(c2)OCCOc2ncnc(C4CC4)c2-c2ncc4cnn(c4n2)C3)C1 ; CN1CC(n2cc(C(F)(F)F)nc2-c2ccc3c(c2)OCCOc2ncnc(C4CC4)c2-c2ncc4cnn(c4n2)C3)C1]

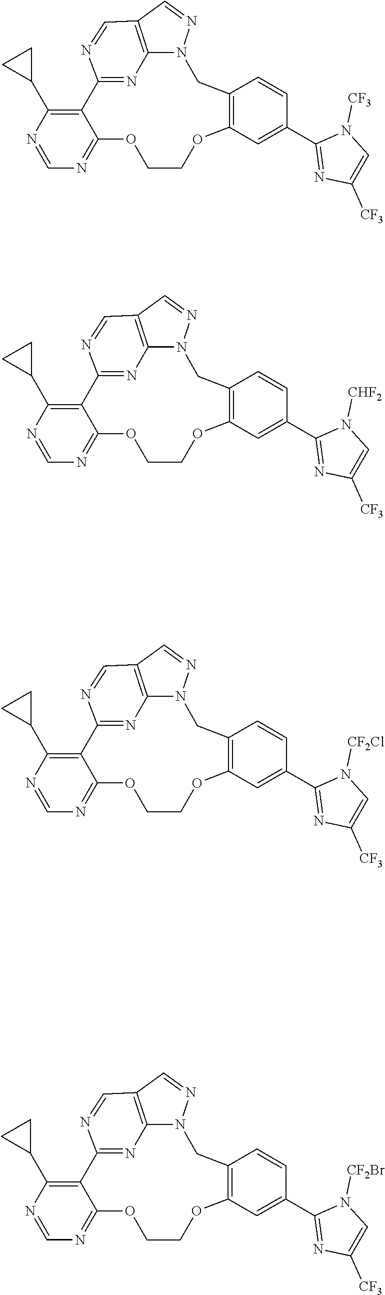

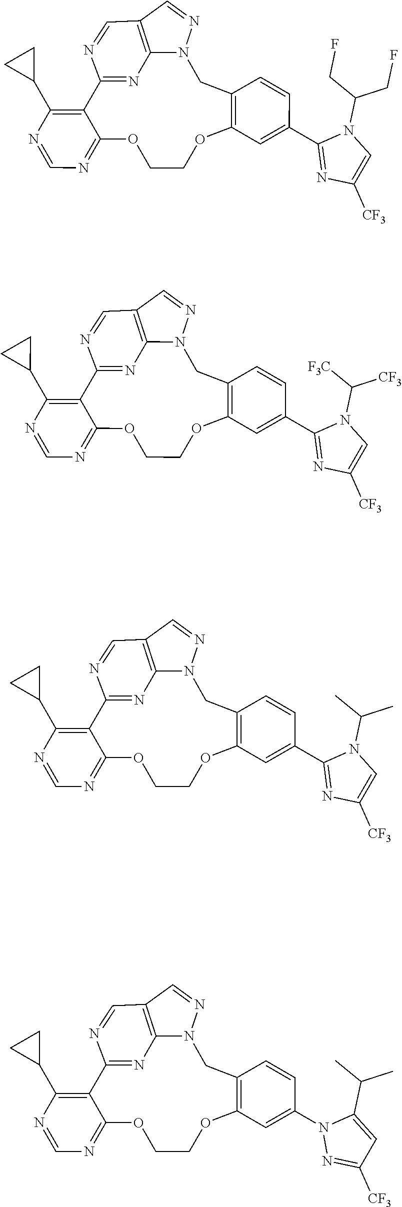 [SMILES: FCC(CF)n1cc(C(F)(F)F)nc1-c1ccc2c(c1)OCCOc1ncnc(C3CC3)c1-c1ncc3cnn(c3n1)C2 ; FC(F)(F)c1cn(C(C(F)(F)F)C(F)(F)F)c(-c2ccc3c(c2)OCCOc2ncnc(C4CC4)c2-c2ncc4cnn(c4n2)C3)n1 ; CC(C)n1cc(C(F)(F)F)nc1-c1ccc2c(c1)OCCOc1ncnc(C3CC3)c1-c1ncc3cnn(c3n1)C2 ; CC(C)c1cc(C(F)(F)F)nn1-c1ccc2c(c1)OCCOc1ncnc(C3CC3)c1-c1ncc3cnn(c3n1)C2]

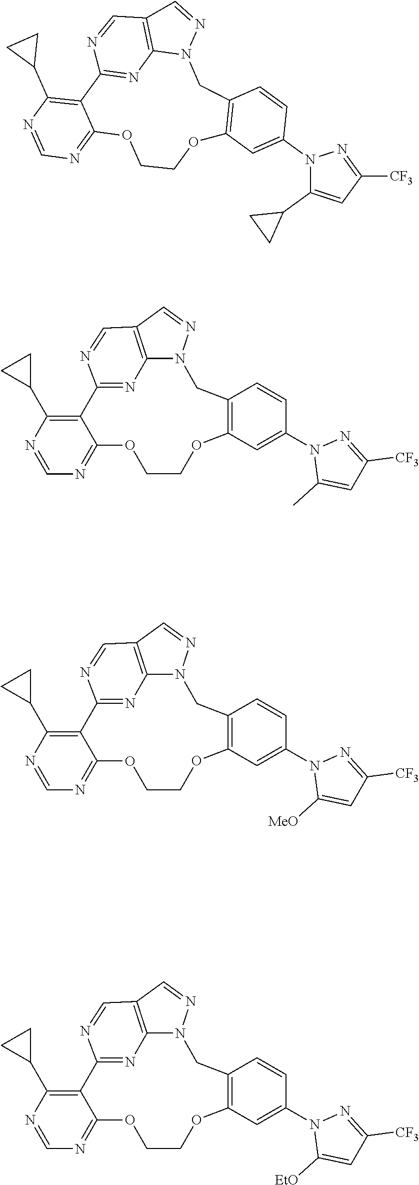 [SMILES: FC(F)(F)c1cc(C2CC2)n(-c2ccc3c(c2)OCCOc2ncnc(C4CC4)c2-c2ncc4cnn(c4n2)C3)n1 ; Cc1cc(C(F)(F)F)nn1-c1ccc2c(c1)OCCOc1ncnc(C3CC3)c1-c1ncc3cnn(c3n1)C2 ; COc1cc(C(F)(F)F)nn1-c1ccc2c(c1)OCCOc1ncnc(C3CC3)c1-c1ncc3cnn(c3n1)C2 ; CCOc1cc(C(F)(F)F)nn1-c1ccc2c(c1)OCCOc1ncnc(C3CC3)c1-c1ncc3cnn(c3n1)C2]

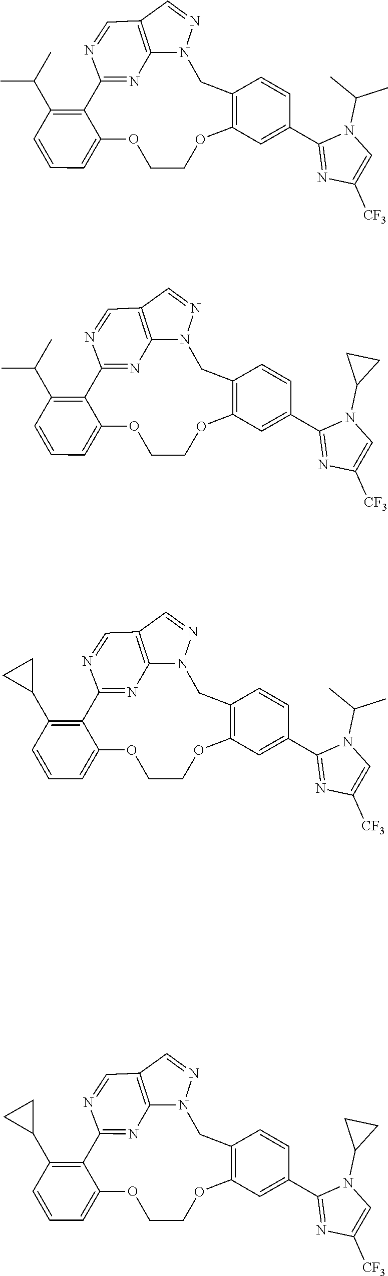

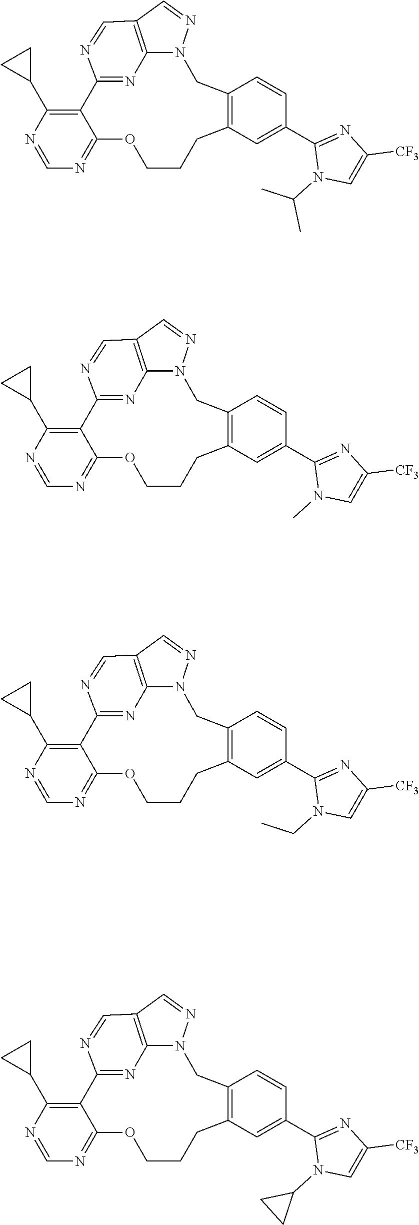 [SMILES: CC(C)n1cc(C(F)(F)F)nc1-c1ccc2c(c1)CCCOc1ncnc(C3CC3)c1-c1ncc3cnn(c3n1)C2 ; Cn1cc(C(F)(F)F)nc1-c1ccc2c(c1)CCCOc1ncnc(C3CC3)c1-c1ncc3cnn(c3n1)C2 ; CCn1cc(C(F)(F)F)nc1-c1ccc2c(c1)CCCOc1ncnc(C3CC3)c1-c1ncc3cnn(c3n1)C2 ; FC(F)(F)c1cn(C2CC2)c(-c2ccc3c(c2)CCCOc2ncnc(C4CC4)c2-c2ncc4cnn(c4n2)C3)n1]

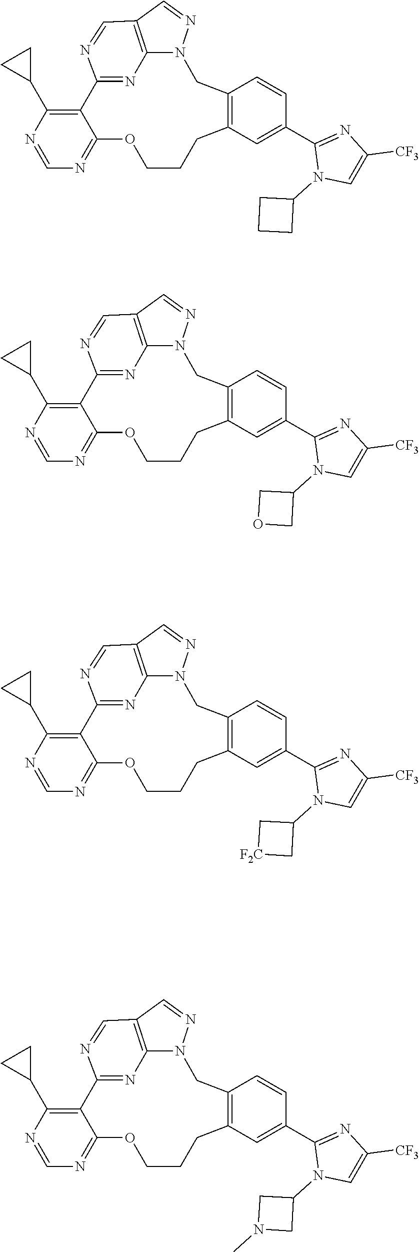 [SMILES: FC(F)(F)c1cn(C2CCC2)c(-c2ccc3c(c2)CCCOc2ncnc(C4CC4)c2-c2ncc4cnn(c4n2)C3)n1 ; FC(F)(F)c1cn(C2COC2)c(-c2ccc3c(c2)CCCOc2ncnc(C4CC4)c2-c2ncc4cnn(c4n2)C3)n1 ; FC1(F)CC(n2cc(C(F)(F)F)nc2-c2ccc3c(c2)CCCOc2ncnc(C4CC4)c2-c2ncc4cnn(c4n2)C3)C1 ; CN1CC(n2cc(C(F)(F)F)nc2-c2ccc3c(c2)CCCOc2ncnc(C4CC4)c2-c2ncc4cnn(c4n2)C3)C1]

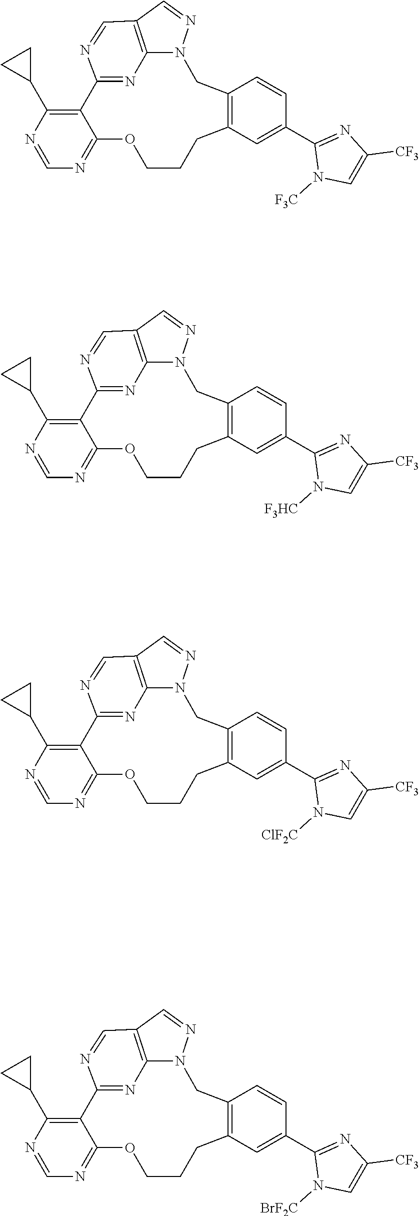

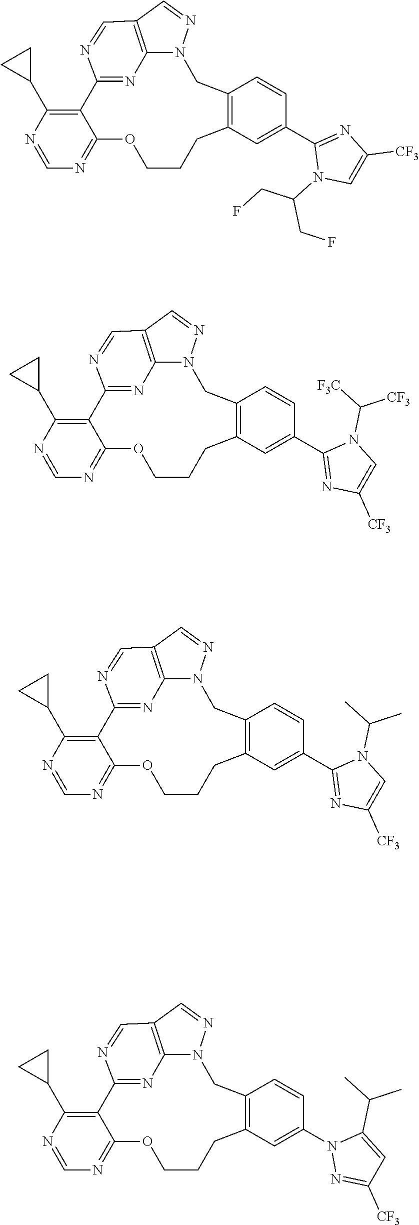 [SMILES: FCC(CF)n1cc(C(F)(F)F)nc1-c1ccc2c(c1)CCCOc1ncnc(C3CC3)c1-c1ncc3cnn(c3n1)C2 ; FC(F)(F)c1cn(C(C(F)(F)F)C(F)(F)F)c(-c2ccc3c(c2)CCCOc2ncnc(C4CC4)c2-c2ncc4cnn(c4n2)C3)n1 ; CC(C)n1cc(C(F)(F)F)nc1-c1ccc2c(c1)CCCOc1ncnc(C3CC3)c1-c1ncc3cnn(c3n1)C2 ; CC(C)c1cc(C(F)(F)F)nn1-c1ccc2c(c1)CCCOc1ncnc(C3CC3)c1-c1ncc3cnn(c3n1)C2]

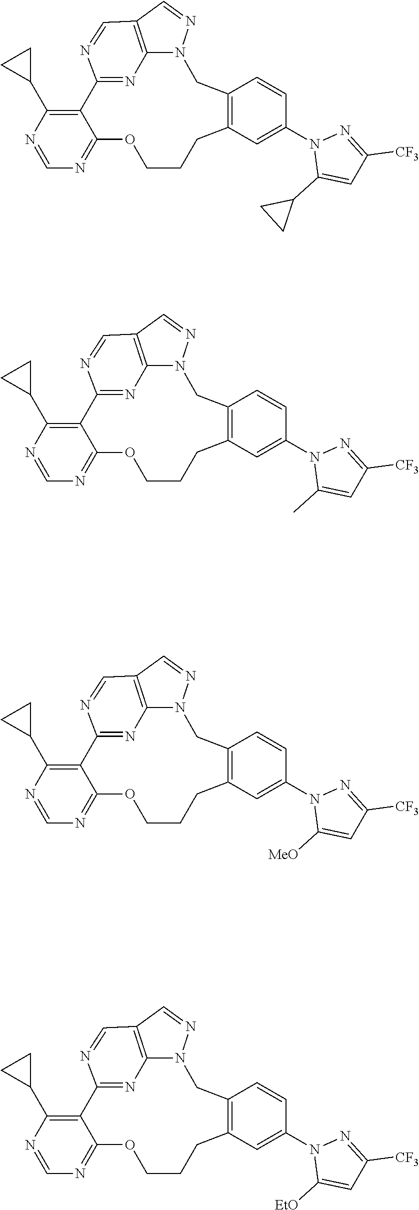 [SMILES: FC(F)(F)c1cc(C2CC2)n(-c2ccc3c(c2)CCCOc2ncnc(C4CC4)c2-c2ncc4cnn(c4n2)C3)n1 ; Cc1cc(C(F)(F)F)nn1-c1ccc2c(c1)CCCOc1ncnc(C3CC3)c1-c1ncc3cnn(c3n1)C2 ; COc1cc(C(F)(F)F)nn1-c1ccc2c(c1)CCCOc1ncnc(C3CC3)c1-c1ncc3cnn(c3n1)C2 ; CCOc1cc(C(F)(F)F)nn1-c1ccc2c(c1)CCCOc1ncnc(C3CC3)c1-c1ncc3cnn(c3n1)C2]

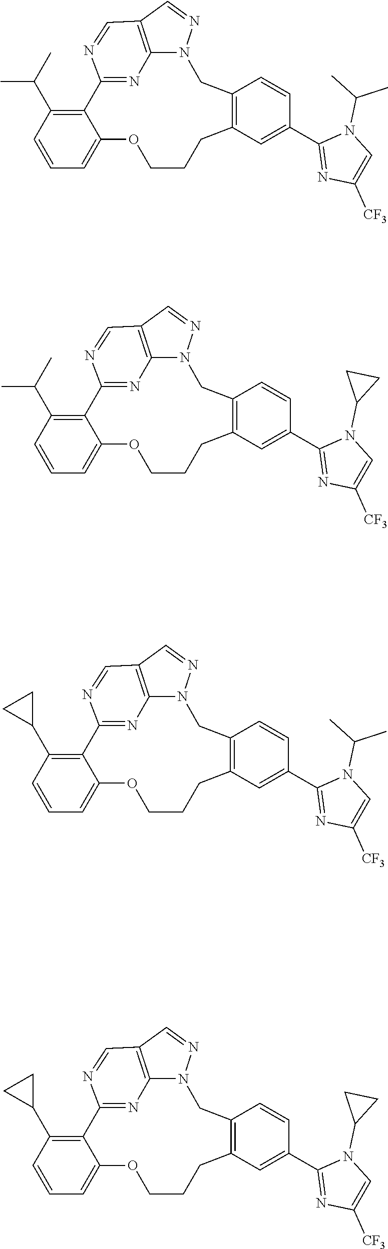

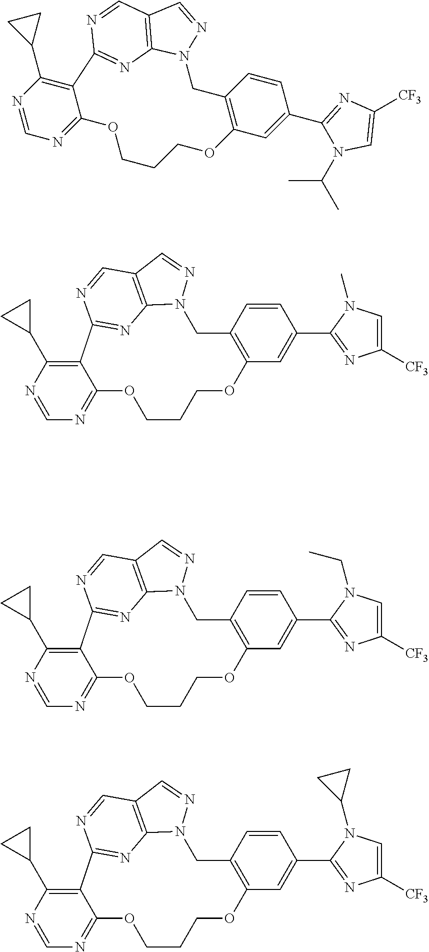 [SMILES: CC(C)n1cc(C(F)(F)F)nc1-c1ccc2c(c1)OCCCOc1ncnc(C3CC3)c1-c1ncc3cnn(c3n1)C2 ; Cn1cc(C(F)(F)F)nc1-c1ccc2c(c1)OCCCOc1ncnc(C3CC3)c1-c1ncc3cnn(c3n1)C2 ; CCn1cc(C(F)(F)F)nc1-c1ccc2c(c1)OCCCOc1ncnc(C3CC3)c1-c1ncc3cnn(c3n1)C2 ; FC(F)(F)c1cn(C2CC2)c(-c2ccc3c(c2)OCCCOc2ncnc(C4CC4)c2-c2ncc4cnn(c4n2)C3)n1]

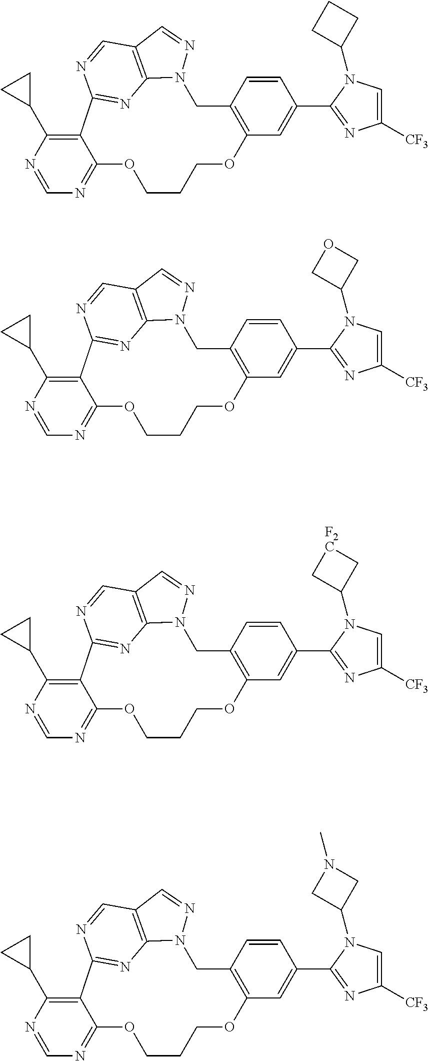 [SMILES: FC(F)(F)c1cn(C2CCC2)c(-c2ccc3c(c2)OCCCOc2ncnc(C4CC4)c2-c2ncc4cnn(c4n2)C3)n1 ; FC(F)(F)c1cn(C2COC2)c(-c2ccc3c(c2)OCCCOc2ncnc(C4CC4)c2-c2ncc4cnn(c4n2)C3)n1 ; FC1(F)CC(n2cc(C(F)(F)F)nc2-c2ccc3c(c2)OCCCOc2ncnc(C4CC4)c2-c2ncc4cnn(c4n2)C3)C1 ; CN1CC(n2cc(C(F)(F)F)nc2-c2ccc3c(c2)OCCCOc2ncnc(C4CC4)c2-c2ncc4cnn(c4n2)C3)C1]

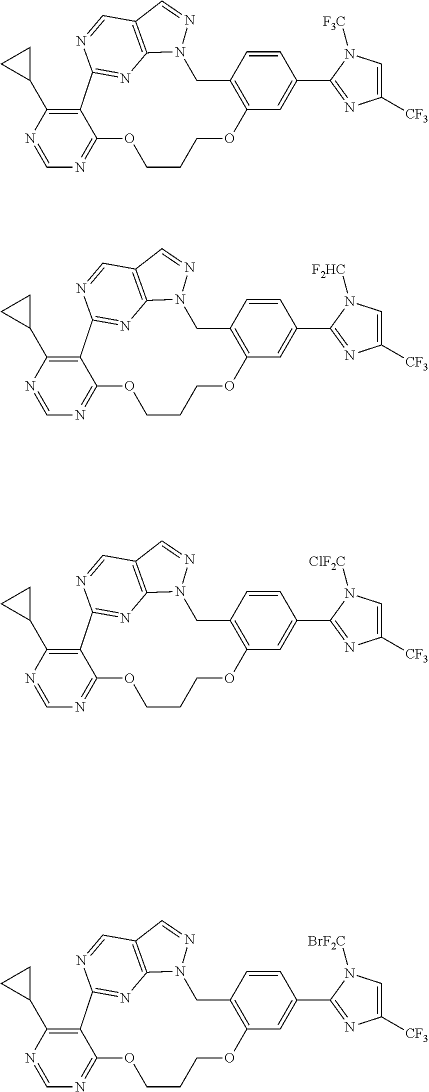

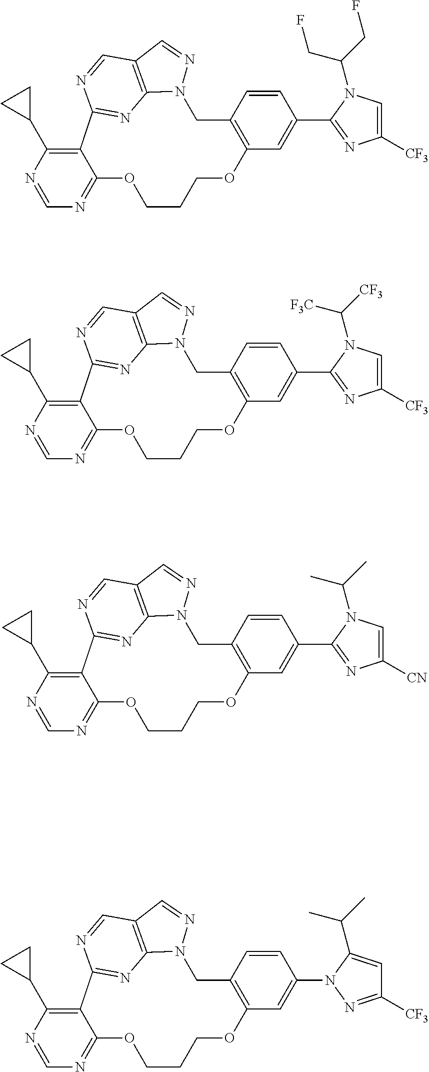 [SMILES: FCC(CF)n1cc(C(F)(F)F)nc1-c1ccc2c(c1)OCCCOc1ncnc(C3CC3)c1-c1ncc3cnn(c3n1)C2 ; FC(F)(F)c1cn(C(C(F)(F)F)C(F)(F)F)c(-c2ccc3c(c2)OCCCOc2ncnc(C4CC4)c2-c2ncc4cnn(c4n2)C3)n1 ; CC(C)n1cc(C#N)nc1-c1ccc2c(c1)OCCCOc1ncnc(C3CC3)c1-c1ncc3cnn(c3n1)C2 ; CC(C)c1cc(C(F)(F)F)nn1-c1ccc2c(c1)OCCCOc1ncnc(C3CC3)c1-c1ncc3cnn(c3n1)C2]

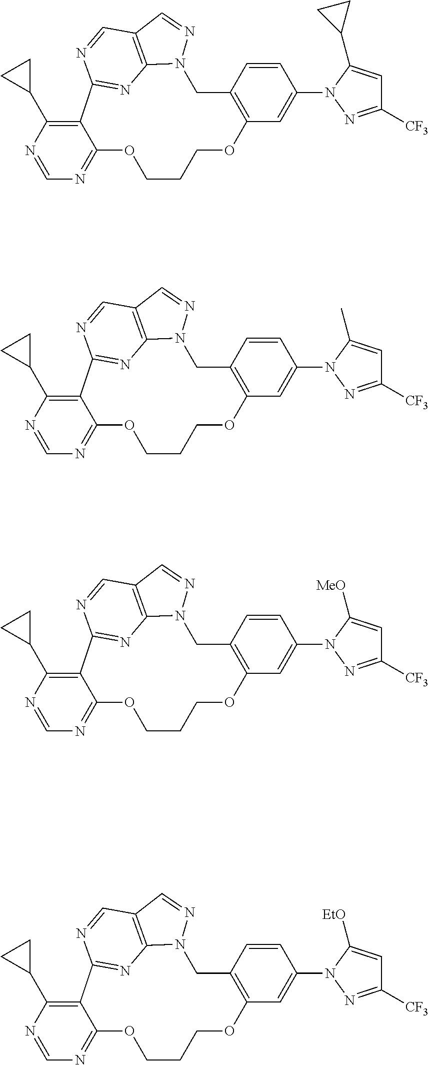 [SMILES: FC(F)(F)c1cc(C2CC2)n(-c2ccc3c(c2)OCCCOc2ncnc(C4CC4)c2-c2ncc4cnn(c4n2)C3)n1 ; Cc1cc(C(F)(F)F)nn1-c1ccc2c(c1)OCCCOc1ncnc(C3CC3)c1-c1ncc3cnn(c3n1)C2 ; COc1cc(C(F)(F)F)nn1-c1ccc2c(c1)OCCCOc1ncnc(C3CC3)c1-c1ncc3cnn(c3n1)C2 ; CCOc1cc(C(F)(F)F)nn1-c1ccc2c(c1)OCCCOc1ncnc(C3CC3)c1-c1ncc3cnn(c3n1)C2]

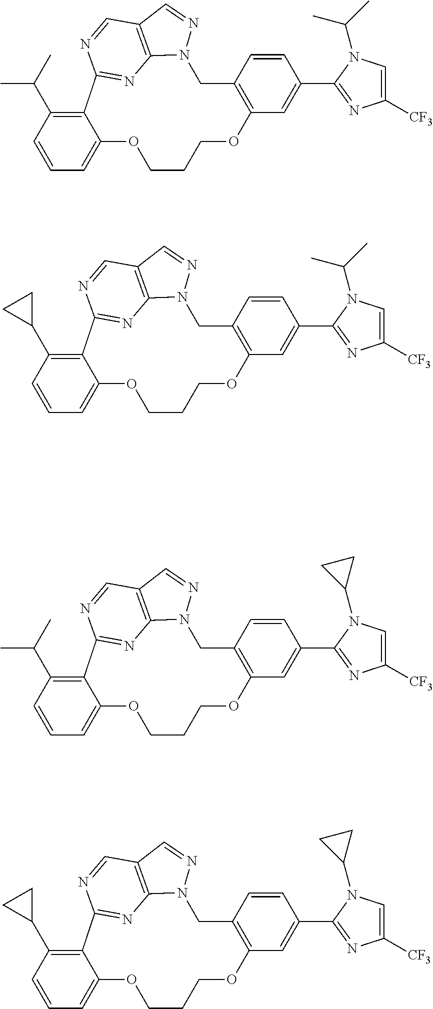 [SMILES: CC(C)c1cccc2c1-c1ncc3cnn(c3n1)Cc1ccc(-c3nc(C(F)(F)F)cn3C(C)C)cc1OCCCO2 ; CC(C)n1cc(C(F)(F)F)nc1-c1ccc2c(c1)OCCCOc1cccc(C3CC3)c1-c1ncc3cnn(c3n1)C2 ; CC(C)c1cccc2c1-c1ncc3cnn(c3n1)Cc1ccc(-c3nc(C(F)(F)F)cn3C3CC3)cc1OCCCO2 ; FC(F)(F)c1cn(C2CC2)c(-c2ccc3c(c2)OCCCOc2cccc(C4CC4)c2-c2ncc4cnn(c4n2)C3)n1]

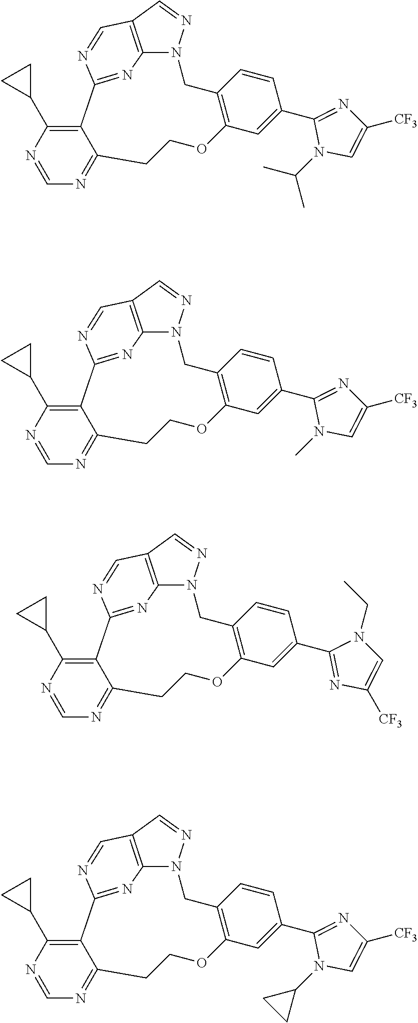 [SMILES: CC(C)n1cc(C(F)(F)F)nc1-c1ccc2c(c1)OCCc1ncnc(C3CC3)c1-c1ncc3cnn(c3n1)C2 ; Cn1cc(C(F)(F)F)nc1-c1ccc2c(c1)OCCc1ncnc(C3CC3)c1-c1ncc3cnn(c3n1)C2 ; CCn1cc(C(F)(F)F)nc1-c1ccc2c(c1)OCCc1ncnc(C3CC3)c1-c1ncc3cnn(c3n1)C2 ; FC(F)(F)c1cn(C2CC2)c(-c2ccc3c(c2)OCCc2ncnc(C4CC4)c2-c2ncc4cnn(c4n2)C3)n1]

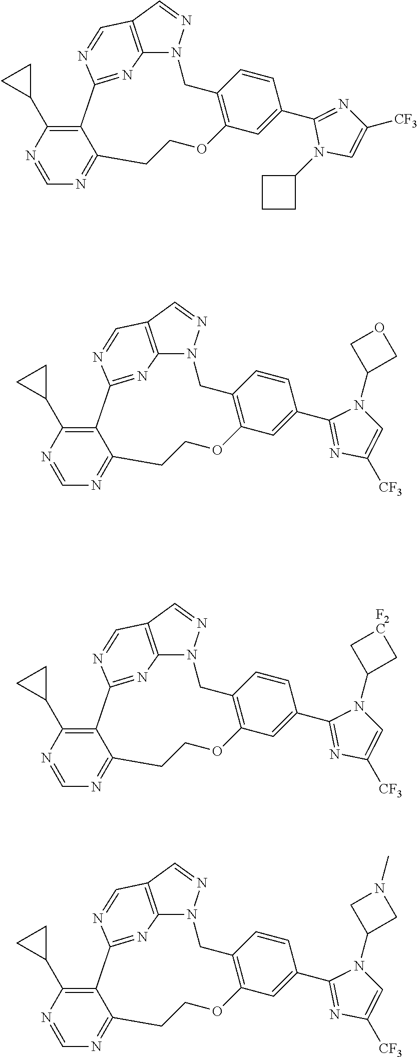 [SMILES: FC(F)(F)c1cn(C2CCC2)c(-c2ccc3c(c2)OCCc2ncnc(C4CC4)c2-c2ncc4cnn(c4n2)C3)n1 ; FC(F)(F)c1cn(C2COC2)c(-c2ccc3c(c2)OCCc2ncnc(C4CC4)c2-c2ncc4cnn(c4n2)C3)n1 ; FC1(F)CC(n2cc(C(F)(F)F)nc2-c2ccc3c(c2)OCCc2ncnc(C4CC4)c2-c2ncc4cnn(c4n2)C3)C1 ; CN1CC(n2cc(C(F)(F)F)nc2-c2ccc3c(c2)OCCc2ncnc(C4CC4)c2-c2ncc4cnn(c4n2)C3)C1]

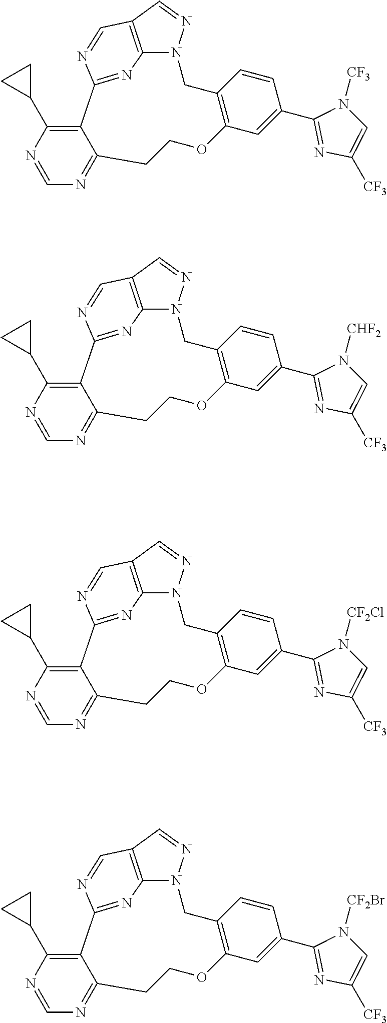

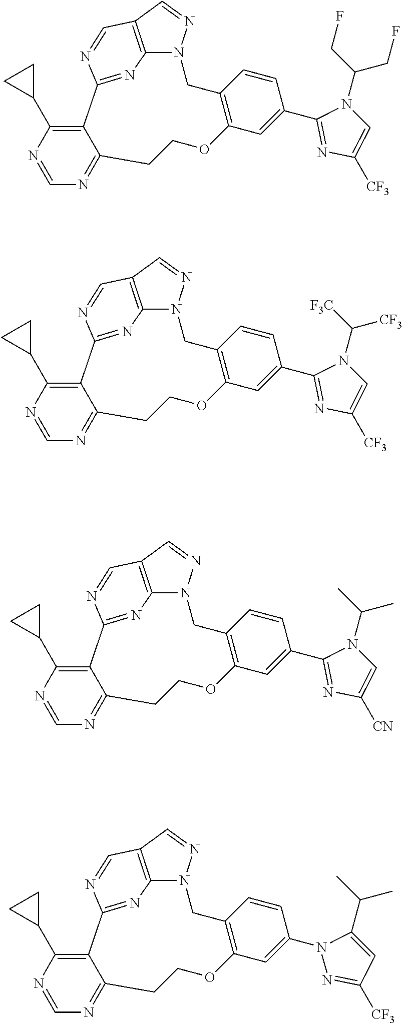 [SMILES: FCC(CF)n1cc(C(F)(F)F)nc1-c1ccc2c(c1)OCCc1ncnc(C3CC3)c1-c1ncc3cnn(c3n1)C2 ; FC(F)(F)c1cn(C(C(F)(F)F)C(F)(F)F)c(-c2ccc3c(c2)OCCc2ncnc(C4CC4)c2-c2ncc4cnn(c4n2)C3)n1 ; CC(C)n1cc(C#N)nc1-c1ccc2c(c1)OCCc1ncnc(C3CC3)c1-c1ncc3cnn(c3n1)C2 ; CC(C)c1cc(C(F)(F)F)nn1-c1ccc2c(c1)OCCc1ncnc(C3CC3)c1-c1ncc3cnn(c3n1)C2]

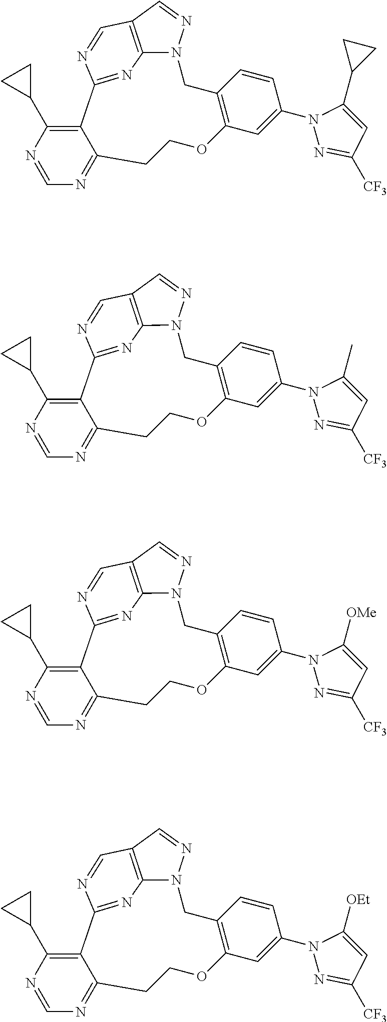 [SMILES: FC(F)(F)c1cc(C2CC2)n(-c2ccc3c(c2)OCCc2ncnc(C4CC4)c2-c2ncc4cnn(c4n2)C3)n1 ; Cc1cc(C(F)(F)F)nn1-c1ccc2c(c1)OCCc1ncnc(C3CC3)c1-c1ncc3cnn(c3n1)C2 ; COc1cc(C(F)(F)F)nn1-c1ccc2c(c1)OCCc1ncnc(C3CC3)c1-c1ncc3cnn(c3n1)C2 ; CCOc1cc(C(F)(F)F)nn1-c1ccc2c(c1)OCCc1ncnc(C3CC3)c1-c1ncc3cnn(c3n1)C2]

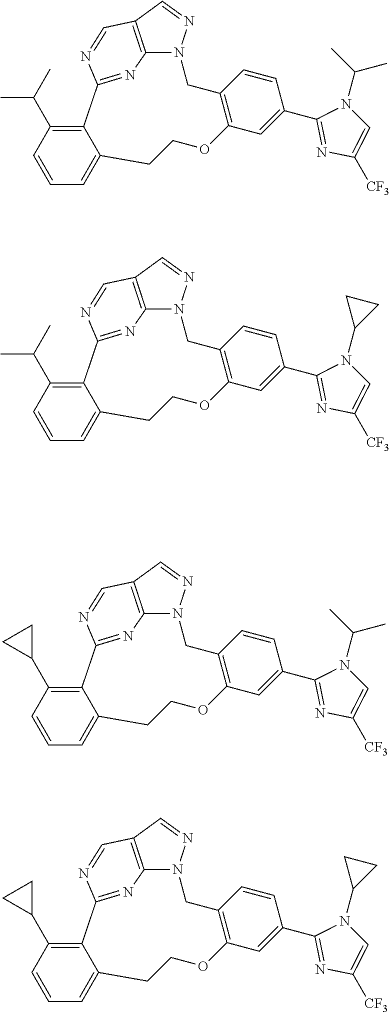

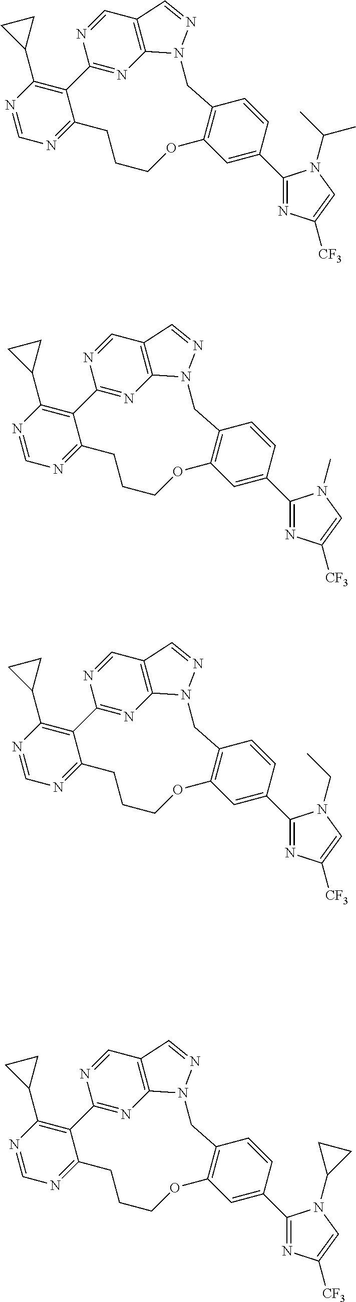 [SMILES: CC(C)n1cc(C(F)(F)F)nc1-c1ccc2c(c1)OCCCc1ncnc(C3CC3)c1-c1ncc3cnn(c3n1)C2 ; Cn1cc(C(F)(F)F)nc1-c1ccc2c(c1)OCCCc1ncnc(C3CC3)c1-c1ncc3cnn(c3n1)C2 ; CCn1cc(C(F)(F)F)nc1-c1ccc2c(c1)OCCCc1ncnc(C3CC3)c1-c1ncc3cnn(c3n1)C2 ; FC(F)(F)c1cn(C2CC2)c(-c2ccc3c(c2)OCCCc2ncnc(C4CC4)c2-c2ncc4cnn(c4n2)C3)n1]

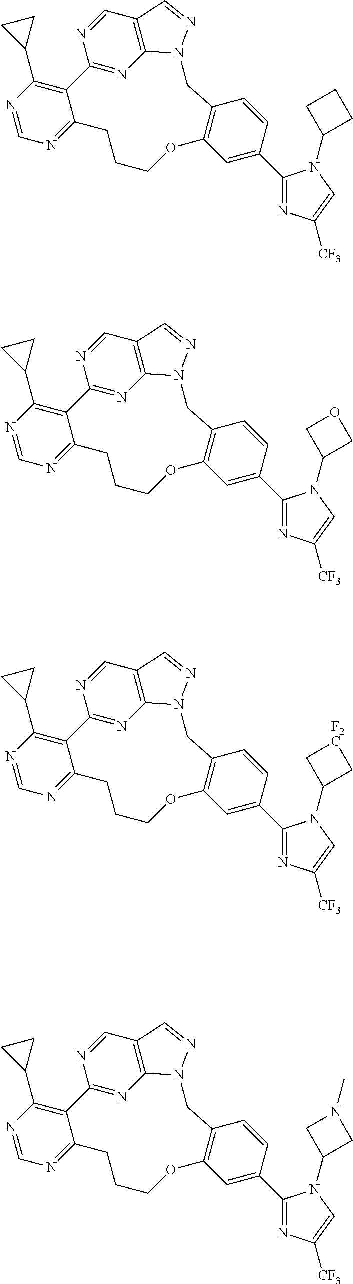 [SMILES: FC(F)(F)c1cn(C2CCC2)c(-c2ccc3c(c2)OCCCc2ncnc(C4CC4)c2-c2ncc4cnn(c4n2)C3)n1 ; FC(F)(F)c1cn(C2COC2)c(-c2ccc3c(c2)OCCCc2ncnc(C4CC4)c2-c2ncc4cnn(c4n2)C3)n1 ; FC1(F)CC(n2cc(C(F)(F)F)nc2-c2ccc3c(c2)OCCCc2ncnc(C4CC4)c2-c2ncc4cnn(c4n2)C3)C1 ; CN1CC(n2cc(C(F)(F)F)nc2-c2ccc3c(c2)OCCCc2ncnc(C4CC4)c2-c2ncc4cnn(c4n2)C3)C1]

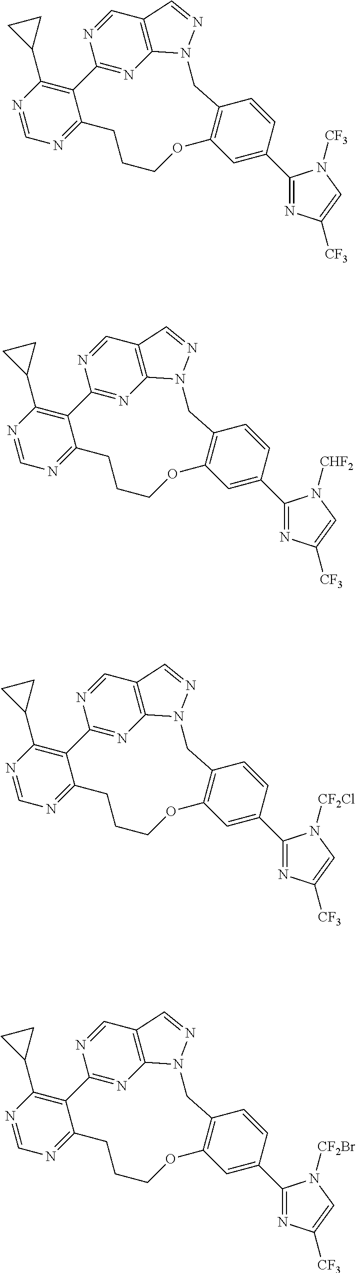

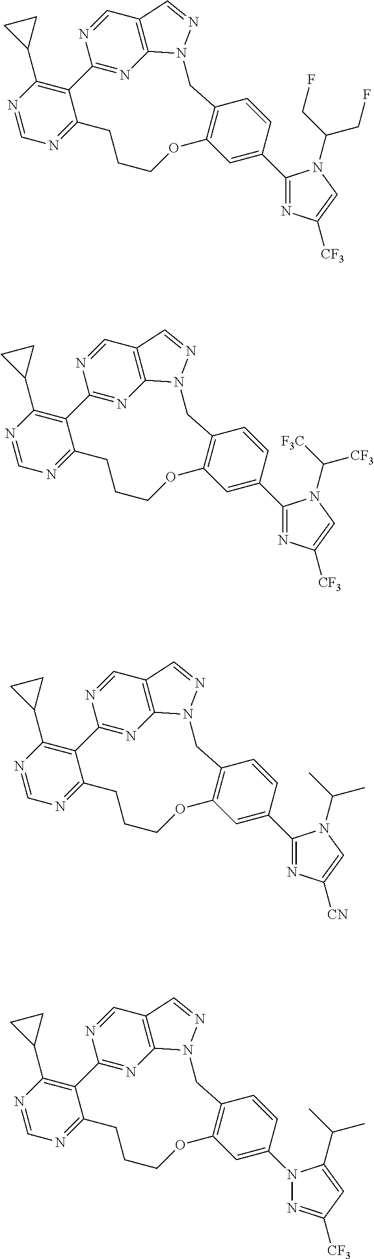 [SMILES: FCC(CF)n1cc(C(F)(F)F)nc1-c1ccc2c(c1)OCCCc1ncnc(C3CC3)c1-c1ncc3cnn(c3n1)C2 ; FC(F)(F)c1cn(C(C(F)(F)F)C(F)(F)F)c(-c2ccc3c(c2)OCCCc2ncnc(C4CC4)c2-c2ncc4cnn(c4n2)C3)n1 ; CC(C)n1cc(C#N)nc1-c1ccc2c(c1)OCCCc1ncnc(C3CC3)c1-c1ncc3cnn(c3n1)C2 ; CC(C)c1cc(C(F)(F)F)nn1-c1ccc2c(c1)OCCCc1ncnc(C3CC3)c1-c1ncc3cnn(c3n1)C2]

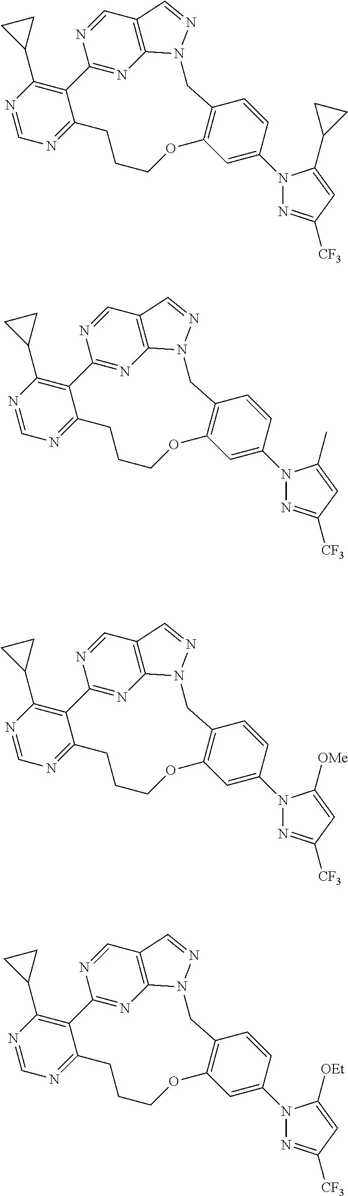 [SMILES: FC(F)(F)c1cc(C2CC2)n(-c2ccc3c(c2)OCCCc2ncnc(C4CC4)c2-c2ncc4cnn(c4n2)C3)n1 ; Cc1cc(C(F)(F)F)nn1-c1ccc2c(c1)OCCCc1ncnc(C3CC3)c1-c1ncc3cnn(c3n1)C2 ; COc1cc(C(F)(F)F)nn1-c1ccc2c(c1)OCCCc1ncnc(C3CC3)c1-c1ncc3cnn(c3n1)C2 ; CCOc1cc(C(F)(F)F)nn1-c1ccc2c(c1)OCCCc1ncnc(C3CC3)c1-c1ncc3cnn(c3n1)C2]

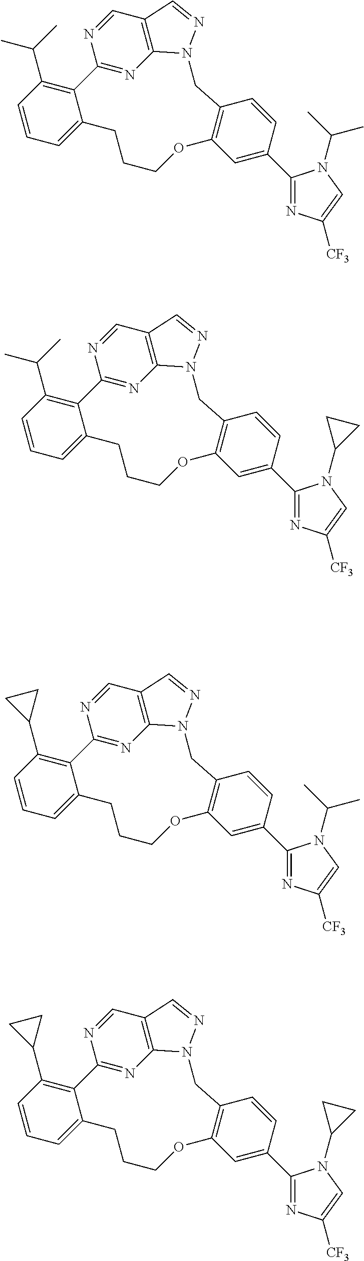

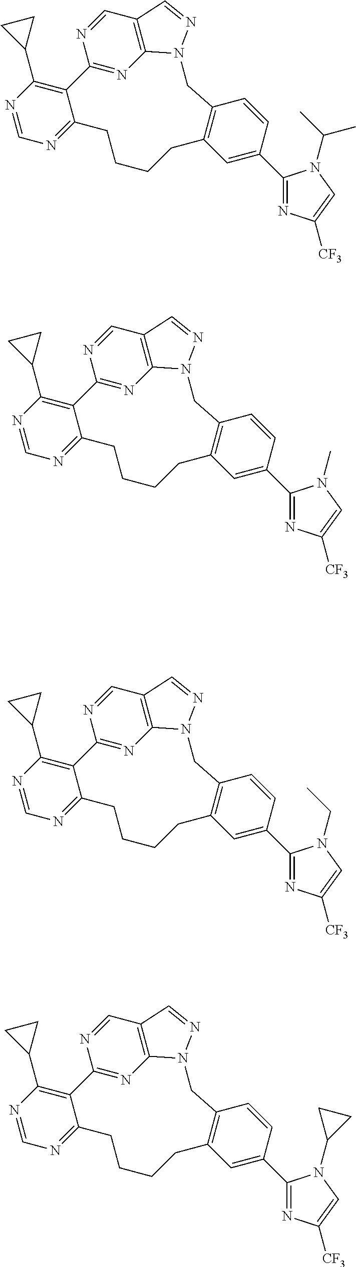 [SMILES: CC(C)n1cc(C(F)(F)F)nc1-c1ccc2c(c1)CCCCc1ncnc(C3CC3)c1-c1ncc3cnn(c3n1)C2 ; Cn1cc(C(F)(F)F)nc1-c1ccc2c(c1)CCCCc1ncnc(C3CC3)c1-c1ncc3cnn(c3n1)C2 ; CCn1cc(C(F)(F)F)nc1-c1ccc2c(c1)CCCCc1ncnc(C3CC3)c1-c1ncc3cnn(c3n1)C2 ; FC(F)(F)c1cn(C2CC2)c(-c2ccc3c(c2)CCCCc2ncnc(C4CC4)c2-c2ncc4cnn(c4n2)C3)n1]

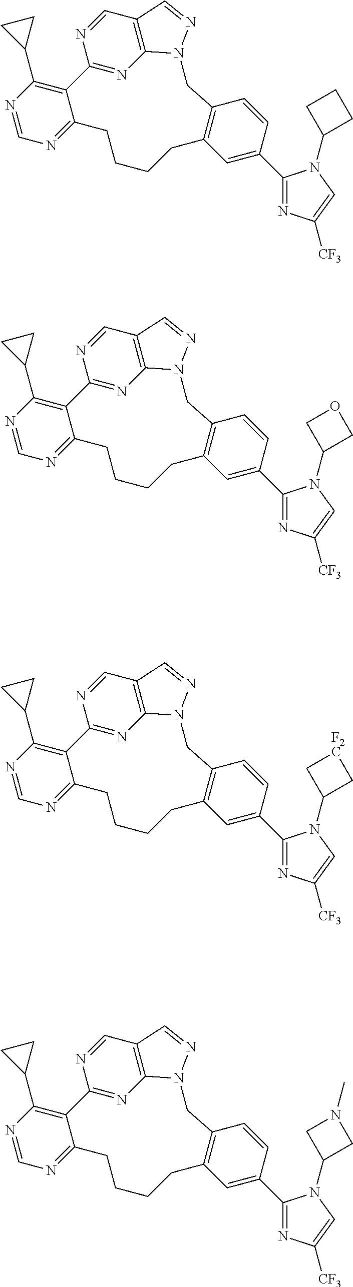 [SMILES: FC(F)(F)c1cn(C2CCC2)c(-c2ccc3c(c2)CCCCc2ncnc(C4CC4)c2-c2ncc4cnn(c4n2)C3)n1 ; FC(F)(F)c1cn(C2COC2)c(-c2ccc3c(c2)CCCCc2ncnc(C4CC4)c2-c2ncc4cnn(c4n2)C3)n1 ; FC1(F)CC(n2cc(C(F)(F)F)nc2-c2ccc3c(c2)CCCCc2ncnc(C4CC4)c2-c2ncc4cnn(c4n2)C3)C1 ; CN1CC(n2cc(C(F)(F)F)nc2-c2ccc3c(c2)CCCCc2ncnc(C4CC4)c2-c2ncc4cnn(c4n2)C3)C1]

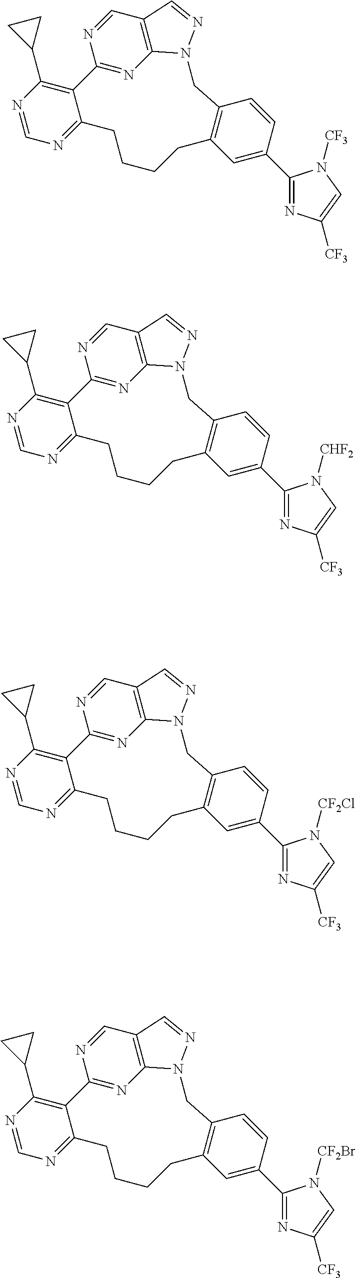

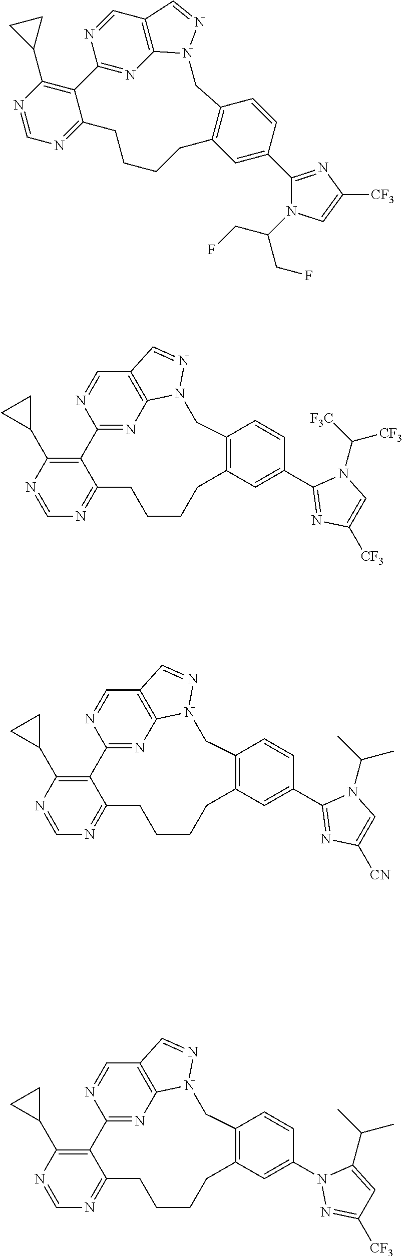 [SMILES: FCC(CF)n1cc(C(F)(F)F)nc1-c1ccc2c(c1)CCCCc1ncnc(C3CC3)c1-c1ncc3cnn(c3n1)C2 ; FC(F)(F)c1cn(C(C(F)(F)F)C(F)(F)F)c(-c2ccc3c(c2)CCCCc2ncnc(C4CC4)c2-c2ncc4cnn(c4n2)C3)n1 ; CC(C)n1cc(C#N)nc1-c1ccc2c(c1)CCCCc1ncnc(C3CC3)c1-c1ncc3cnn(c3n1)C2 ; CC(C)c1cc(C(F)(F)F)nn1-c1ccc2c(c1)CCCCc1ncnc(C3CC3)c1-c1ncc3cnn(c3n1)C2]

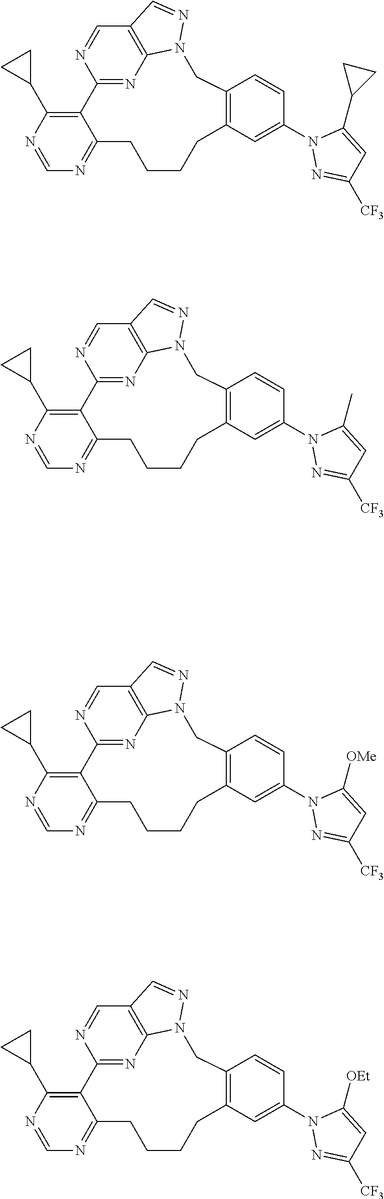 [SMILES: FC(F)(F)c1cc(C2CC2)n(-c2ccc3c(c2)CCCCc2ncnc(C4CC4)c2-c2ncc4cnn(c4n2)C3)n1 ; Cc1cc(C(F)(F)F)nn1-c1ccc2c(c1)CCCCc1ncnc(C3CC3)c1-c1ncc3cnn(c3n1)C2 ; COc1cc(C(F)(F)F)nn1-c1ccc2c(c1)CCCCc1ncnc(C3CC3)c1-c1ncc3cnn(c3n1)C2 ; CCOc1cc(C(F)(F)F)nn1-c1ccc2c(c1)CCCCc1ncnc(C3CC3)c1-c1ncc3cnn(c3n1)C2]

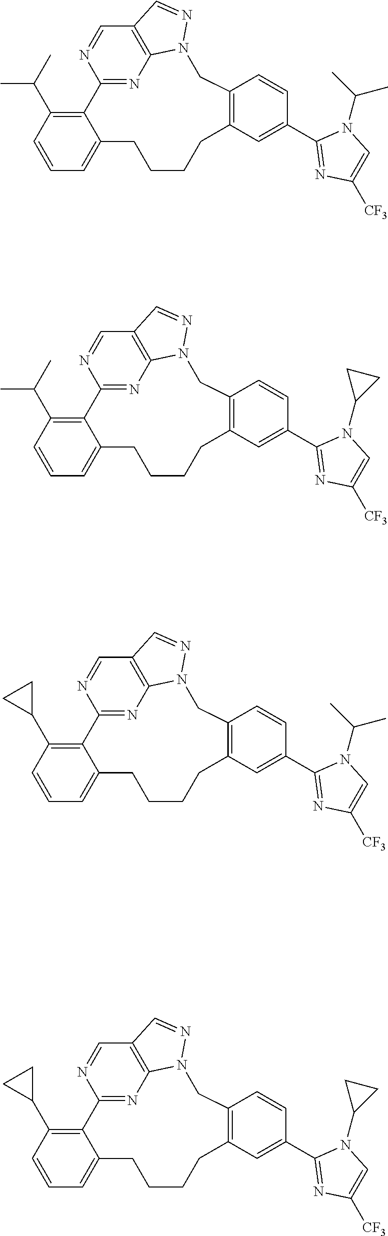

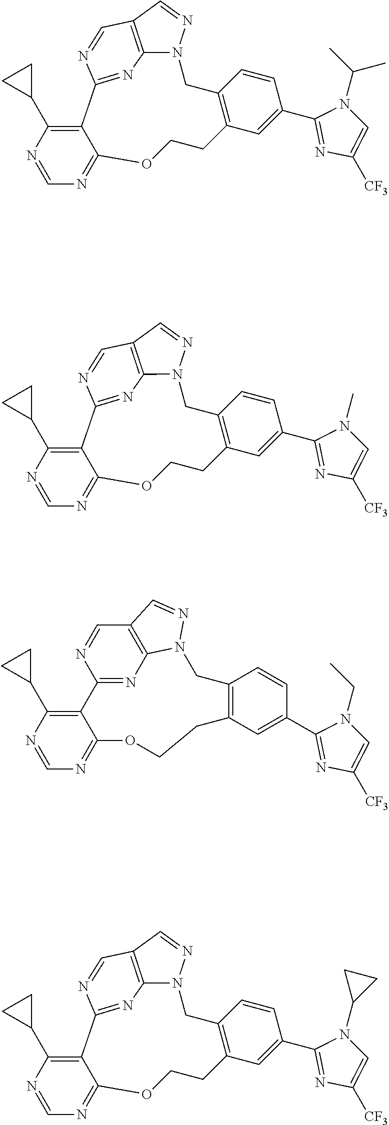 [SMILES: CC(C)n1cc(C(F)(F)F)nc1-c1ccc2c(c1)CCOc1ncnc(C3CC3)c1-c1ncc3cnn(c3n1)C2 ; Cn1cc(C(F)(F)F)nc1-c1ccc2c(c1)CCOc1ncnc(C3CC3)c1-c1ncc3cnn(c3n1)C2 ; CCn1cc(C(F)(F)F)nc1-c1ccc2c(c1)CCOc1ncnc(C3CC3)c1-c1ncc3cnn(c3n1)C2 ; FC(F)(F)c1cn(C2CC2)c(-c2ccc3c(c2)CCOc2ncnc(C4CC4)c2-c2ncc4cnn(c4n2)C3)n1]

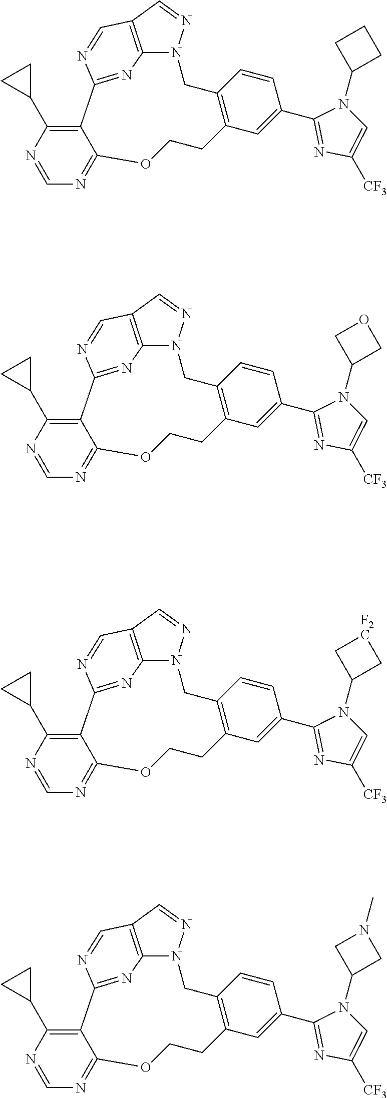 [SMILES: FC(F)(F)c1cn(C2CCC2)c(-c2ccc3c(c2)CCOc2ncnc(C4CC4)c2-c2ncc4cnn(c4n2)C3)n1 ; FC(F)(F)c1cn(C2COC2)c(-c2ccc3c(c2)CCOc2ncnc(C4CC4)c2-c2ncc4cnn(c4n2)C3)n1 ; FC1(F)CC(n2cc(C(F)(F)F)nc2-c2ccc3c(c2)CCOc2ncnc(C4CC4)c2-c2ncc4cnn(c4n2)C3)C1 ; CN1CC(n2cc(C(F)(F)F)nc2-c2ccc3c(c2)CCOc2ncnc(C4CC4)c2-c2ncc4cnn(c4n2)C3)C1]

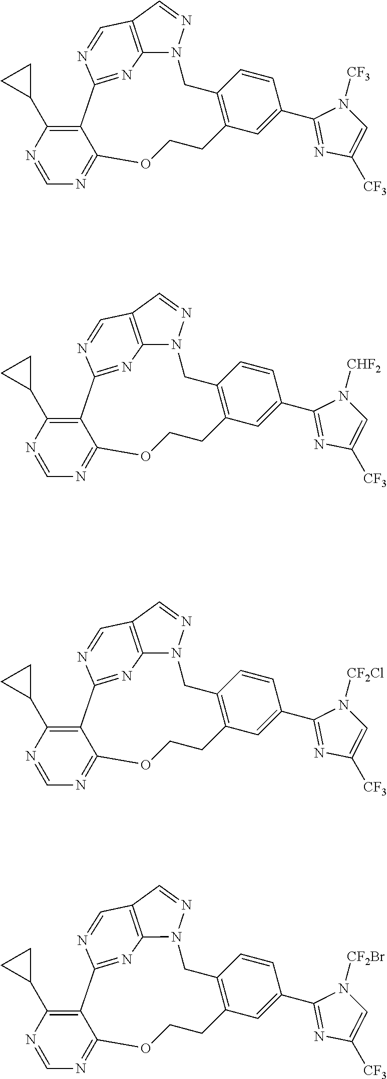

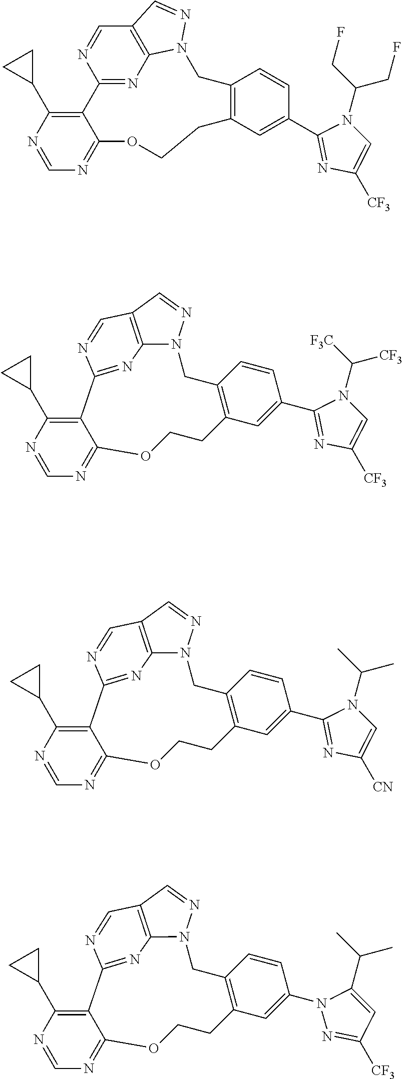 [SMILES: FCC(CF)n1cc(C(F)(F)F)nc1-c1ccc2c(c1)CCOc1ncnc(C3CC3)c1-c1ncc3cnn(c3n1)C2 ; FC(F)(F)c1cn(C(C(F)(F)F)C(F)(F)F)c(-c2ccc3c(c2)CCOc2ncnc(C4CC4)c2-c2ncc4cnn(c4n2)C3)n1 ; CC(C)n1cc(C#N)nc1-c1ccc2c(c1)CCOc1ncnc(C3CC3)c1-c1ncc3cnn(c3n1)C2 ; CC(C)c1cc(C(F)(F)F)nn1-c1ccc2c(c1)CCOc1ncnc(C3CC3)c1-c1ncc3cnn(c3n1)C2]

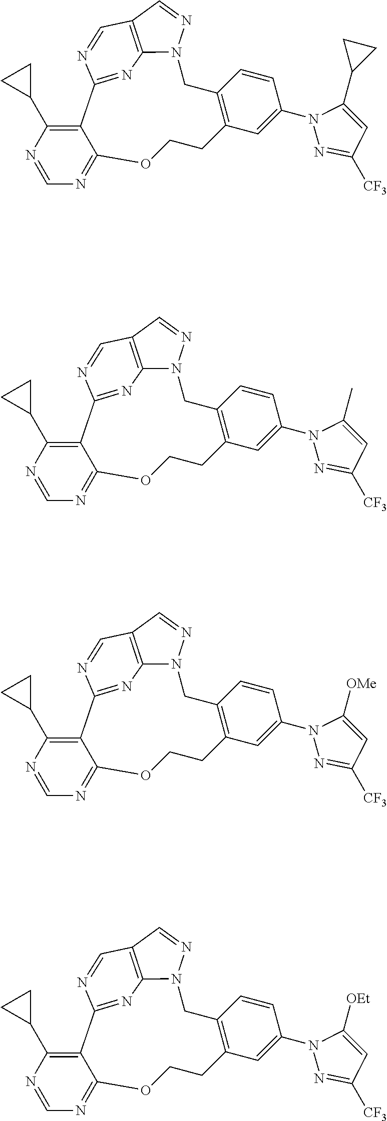 [SMILES: FC(F)(F)c1cc(C2CC2)n(-c2ccc3c(c2)CCOc2ncnc(C4CC4)c2-c2ncc4cnn(c4n2)C3)n1 ; Cc1cc(C(F)(F)F)nn1-c1ccc2c(c1)CCOc1ncnc(C3CC3)c1-c1ncc3cnn(c3n1)C2 ; COc1cc(C(F)(F)F)nn1-c1ccc2c(c1)CCOc1ncnc(C3CC3)c1-c1ncc3cnn(c3n1)C2 ; CCOc1cc(C(F)(F)F)nn1-c1ccc2c(c1)CCOc1ncnc(C3CC3)c1-c1ncc3cnn(c3n1)C2]

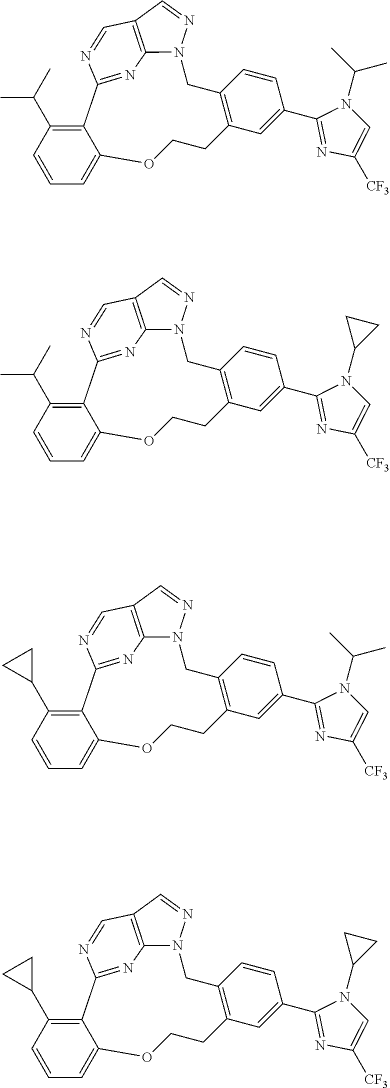

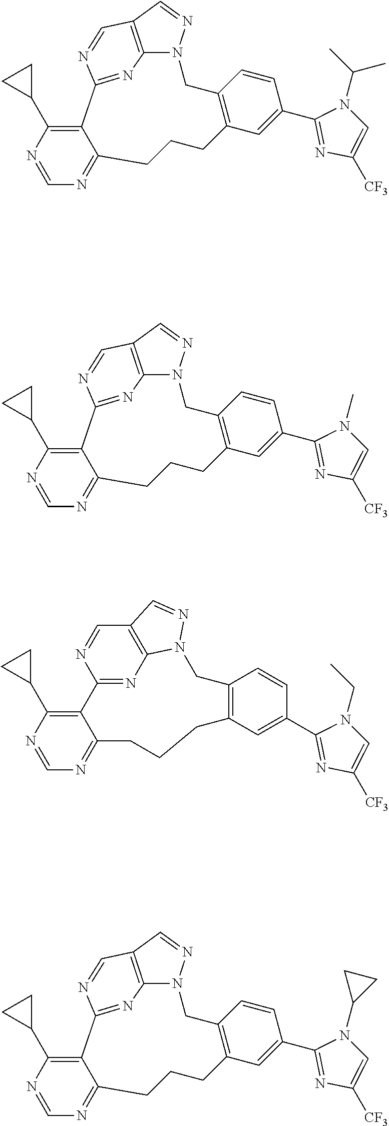 [SMILES: CC(C)n1cc(C(F)(F)F)nc1-c1ccc2c(c1)CCCc1ncnc(C3CC3)c1-c1ncc3cnn(c3n1)C2 ; Cn1cc(C(F)(F)F)nc1-c1ccc2c(c1)CCCc1ncnc(C3CC3)c1-c1ncc3cnn(c3n1)C2 ; CCn1cc(C(F)(F)F)nc1-c1ccc2c(c1)CCCc1ncnc(C3CC3)c1-c1ncc3cnn(c3n1)C2 ; FC(F)(F)c1cn(C2CC2)c(-c2ccc3c(c2)CCCc2ncnc(C4CC4)c2-c2ncc4cnn(c4n2)C3)n1]

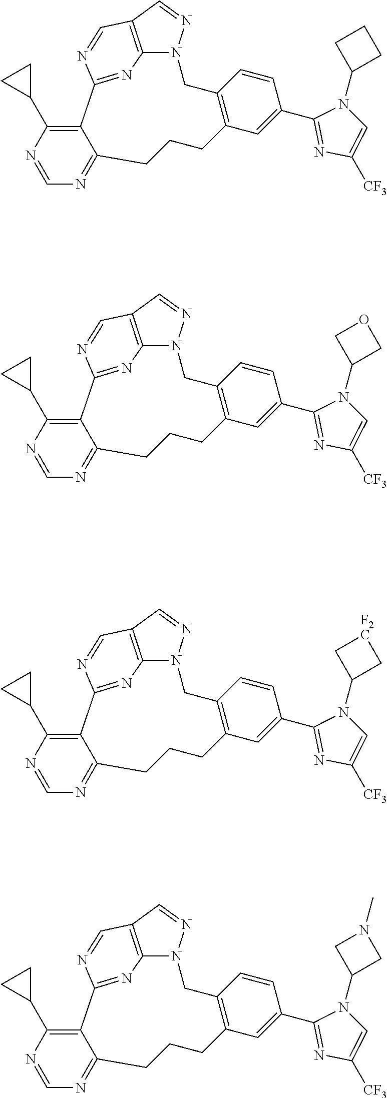 [SMILES: FC(F)(F)c1cn(C2CCC2)c(-c2ccc3c(c2)CCCc2ncnc(C4CC4)c2-c2ncc4cnn(c4n2)C3)n1 ; FC(F)(F)c1cn(C2COC2)c(-c2ccc3c(c2)CCCc2ncnc(C4CC4)c2-c2ncc4cnn(c4n2)C3)n1 ; FC1(F)CC(n2cc(C(F)(F)F)nc2-c2ccc3c(c2)CCCc2ncnc(C4CC4)c2-c2ncc4cnn(c4n2)C3)C1 ; CN1CC(n2cc(C(F)(F)F)nc2-c2ccc3c(c2)CCCc2ncnc(C4CC4)c2-c2ncc4cnn(c4n2)C3)C1]

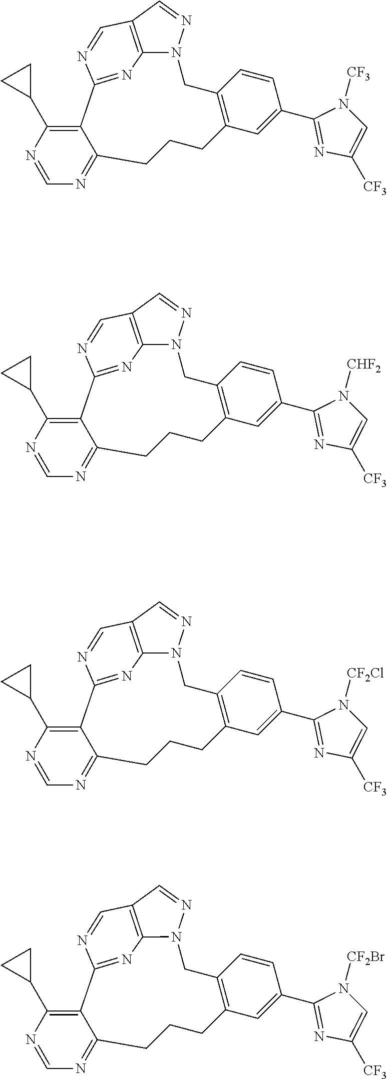

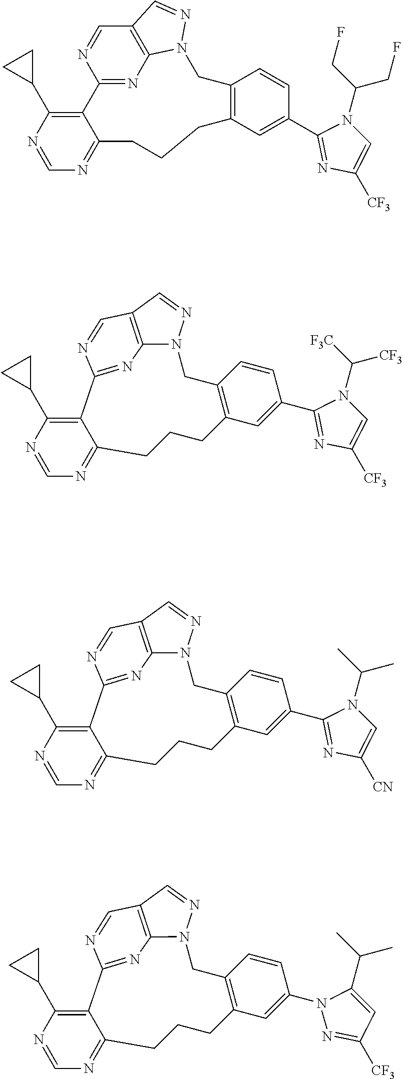 [SMILES: FCC(CF)n1cc(C(F)(F)F)nc1-c1ccc2c(c1)CCCc1ncnc(C3CC3)c1-c1ncc3cnn(c3n1)C2 ; FC(F)(F)c1cn(C(C(F)(F)F)C(F)(F)F)c(-c2ccc3c(c2)CCCc2ncnc(C4CC4)c2-c2ncc4cnn(c4n2)C3)n1 ; CC(C)n1cc(C#N)nc1-c1ccc2c(c1)CCCc1ncnc(C3CC3)c1-c1ncc3cnn(c3n1)C2 ; CC(C)c1cc(C(F)(F)F)nn1-c1ccc2c(c1)CCCc1ncnc(C3CC3)c1-c1ncc3cnn(c3n1)C2]

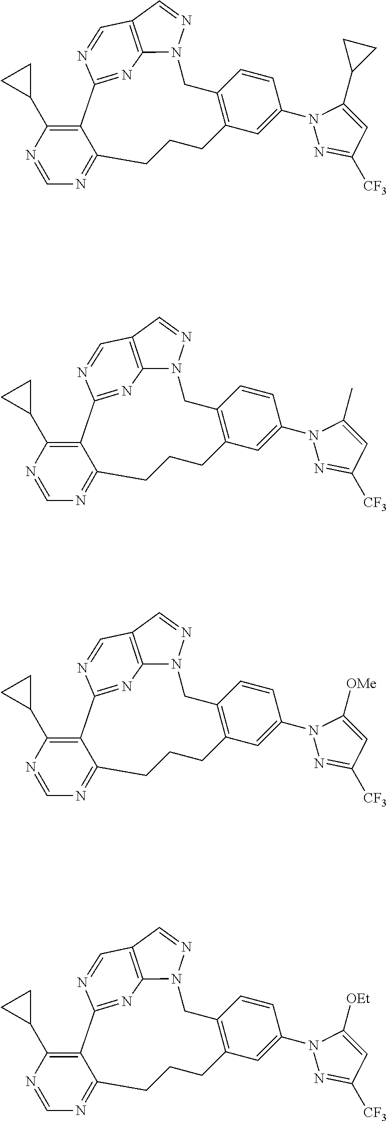 [SMILES: FC(F)(F)c1cc(C2CC2)n(-c2ccc3c(c2)CCCc2ncnc(C4CC4)c2-c2ncc4cnn(c4n2)C3)n1 ; Cc1cc(C(F)(F)F)nn1-c1ccc2c(c1)CCCc1ncnc(C3CC3)c1-c1ncc3cnn(c3n1)C2 ; COc1cc(C(F)(F)F)nn1-c1ccc2c(c1)CCCc1ncnc(C3CC3)c1-c1ncc3cnn(c3n1)C2 ; CCOc1cc(C(F)(F)F)nn1-c1ccc2c(c1)CCCc1ncnc(C3CC3)c1-c1ncc3cnn(c3n1)C2]

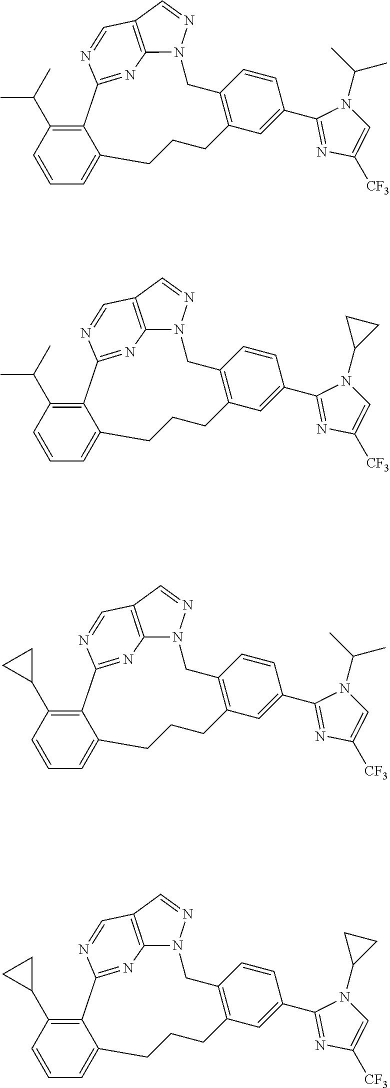

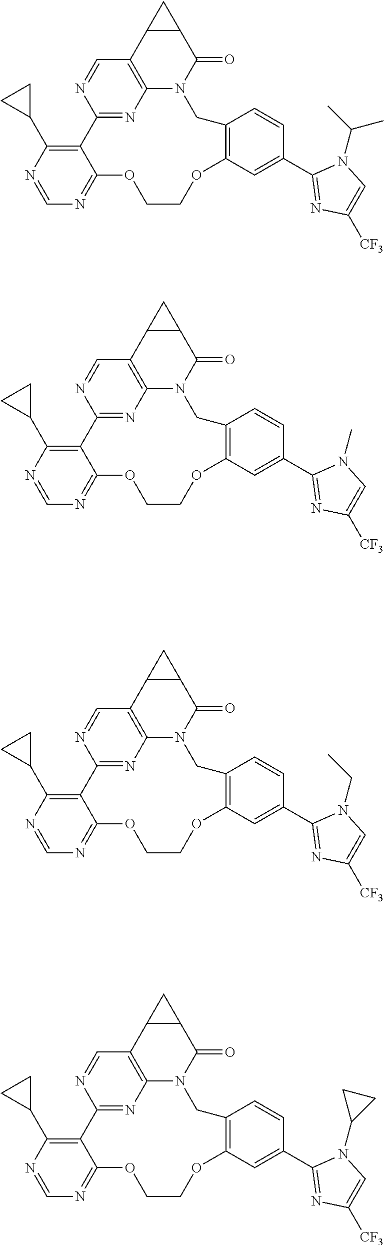 [SMILES: CC(C)n1cc(C(F)(F)F)nc1-c1ccc2c(c1)OCCOc1ncnc(C3CC3)c1-c1ncc3c(n1)N(C2)C(=O)C1CC31 ; Cn1cc(C(F)(F)F)nc1-c1ccc2c(c1)OCCOc1ncnc(C3CC3)c1-c1ncc3c(n1)N(C2)C(=O)C1CC31 ; CCn1cc(C(F)(F)F)nc1-c1ccc2c(c1)OCCOc1ncnc(C3CC3)c1-c1ncc3c(n1)N(C2)C(=O)C1CC31 ; O=C1C2CC2c2cnc3nc2N1Cc1ccc(-c2nc(C(F)(F)F)cn2C2CC2)cc1OCCOc1ncnc(C2CC2)c1-3]

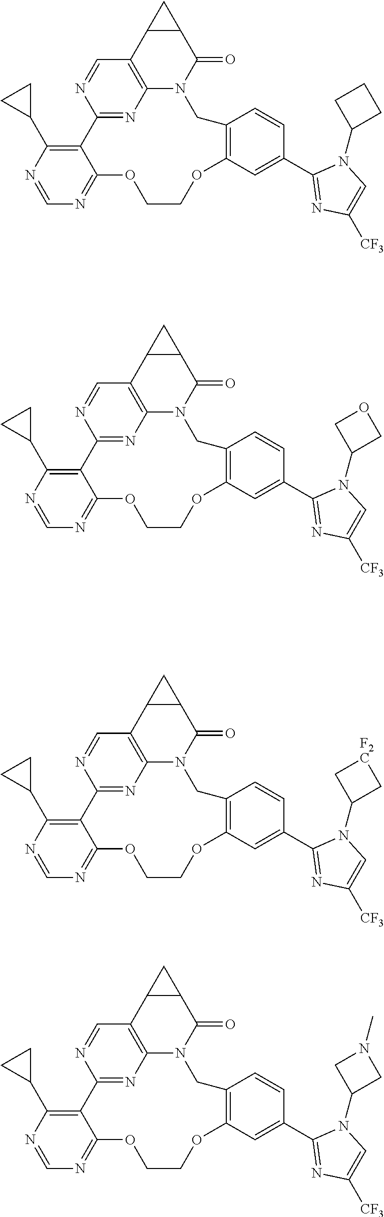 [SMILES: O=C1C2CC2c2cnc3nc2N1Cc1ccc(-c2nc(C(F)(F)F)cn2C2CCC2)cc1OCCOc1ncnc(C2CC2)c1-3 ; O=C1C2CC2c2cnc3nc2N1Cc1ccc(-c2nc(C(F)(F)F)cn2C2COC2)cc1OCCOc1ncnc(C2CC2)c1-3 ; O=C1C2CC2c2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)C2CC42)C1]

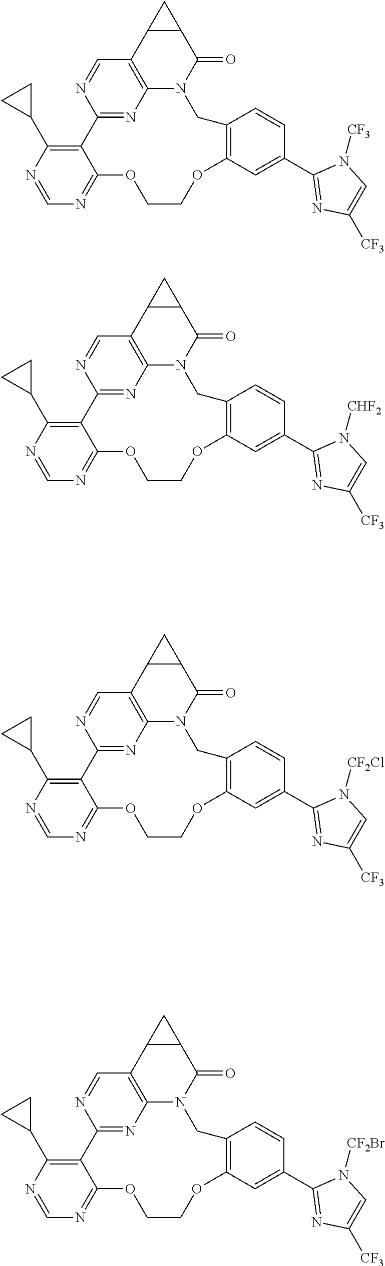

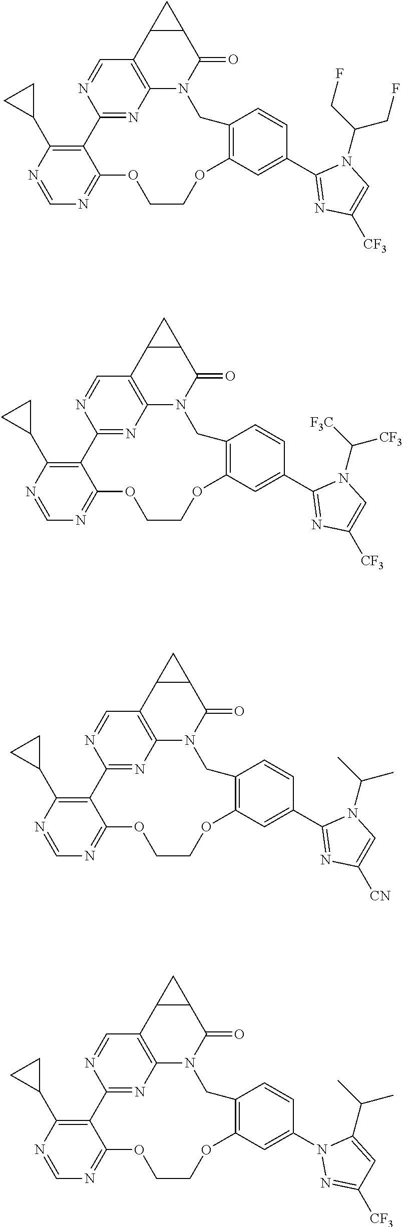 [SMILES: O=C1C2CC2c2cnc3nc2N1Cc1ccc(-c2nc(C(F)(F)F)cn2C(CF)CF)cc1OCCOc1ncnc(C2CC2)c1-3 ; O=C1C2CC2c2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)C1CC31 ; CC(C)c1cc(C(F)(F)F)nn1-c1ccc2c(c1)OCCOc1ncnc(C3CC3)c1-c1ncc3c(n1)N(C2)C(=O)C1CC31]

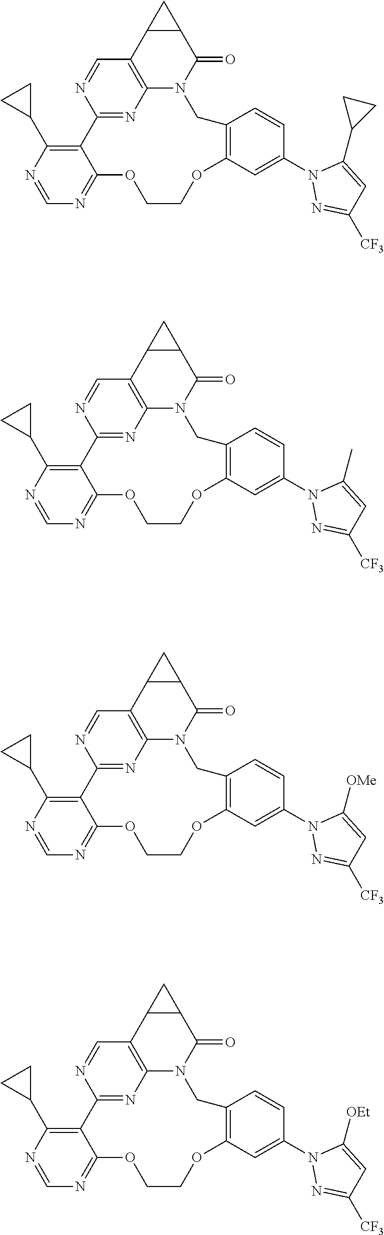 [SMILES: O=C1C2CC2c2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)C1CC31 ; COc1cc(C(F)(F)F)nn1-c1ccc2c(c1)OCCOc1ncnc(C3CC3)c1-c1ncc3c(n1)N(C2)C(=O)C1CC31 ; CCOc1cc(C(F)(F)F)nn1-c1ccc2c(c1)OCCOc1ncnc(C3CC3)c1-c1ncc3c(n1)N(C2)C(=O)C1CC31]

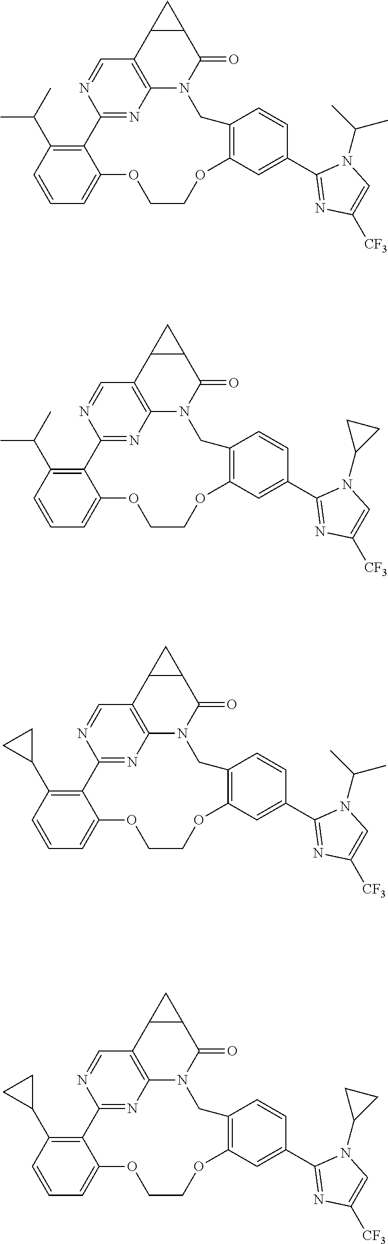

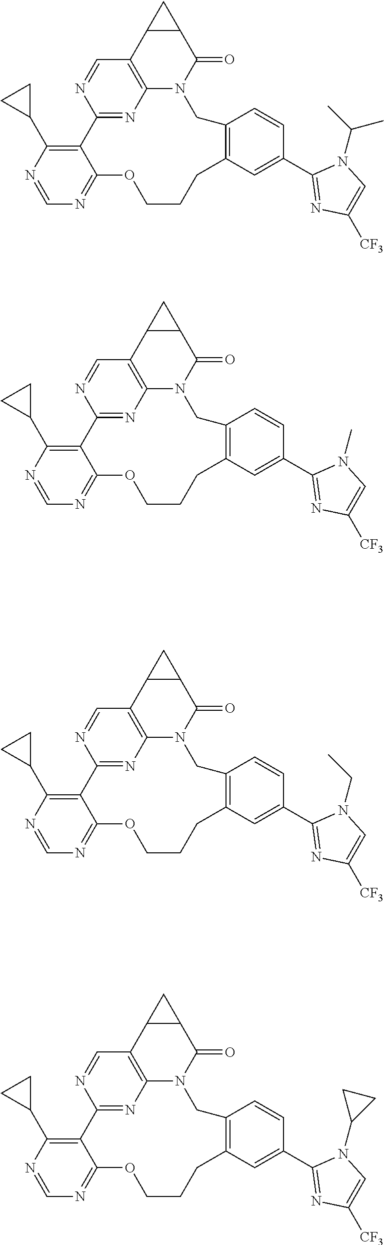 [SMILES: CC(C)n1cc(C(F)(F)F)nc1-c1ccc2c(c1)CCCOc1ncnc(C3CC3)c1-c1ncc3c(n1)N(C2)C(=O)C1CC31 ; Cn1cc(C(F)(F)F)nc1-c1ccc2c(c1)CCCOc1ncnc(C3CC3)c1-c1ncc3c(n1)N(C2)C(=O)C1CC31 ; CCn1cc(C(F)(F)F)nc1-c1ccc2c(c1)CCCOc1ncnc(C3CC3)c1-c1ncc3c(n1)N(C2)C(=O)C1CC31 ; O=C1C2CC2c2cnc3nc2N1Cc1ccc(-c2nc(C(F)(F)F)cn2C2CC2)cc1CCCOc1ncnc(C2CC2)c1-3]

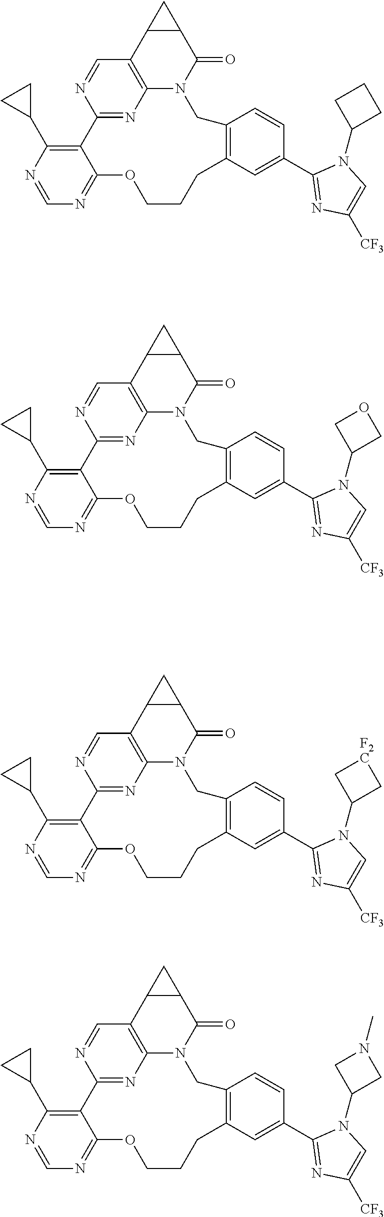 [SMILES: O=C1C2CC2c2cnc3nc2N1Cc1ccc(-c2nc(C(F)(F)F)cn2C2CCC2)cc1CCCOc1ncnc(C2CC2)c1-3 ; O=C1C2CC2c2cnc3nc2N1Cc1ccc(-c2nc(C(F)(F)F)cn2C2COC2)cc1CCCOc1ncnc(C2CC2)c1-3 ; O=C1C2CC2c2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)C2CC42)C1]

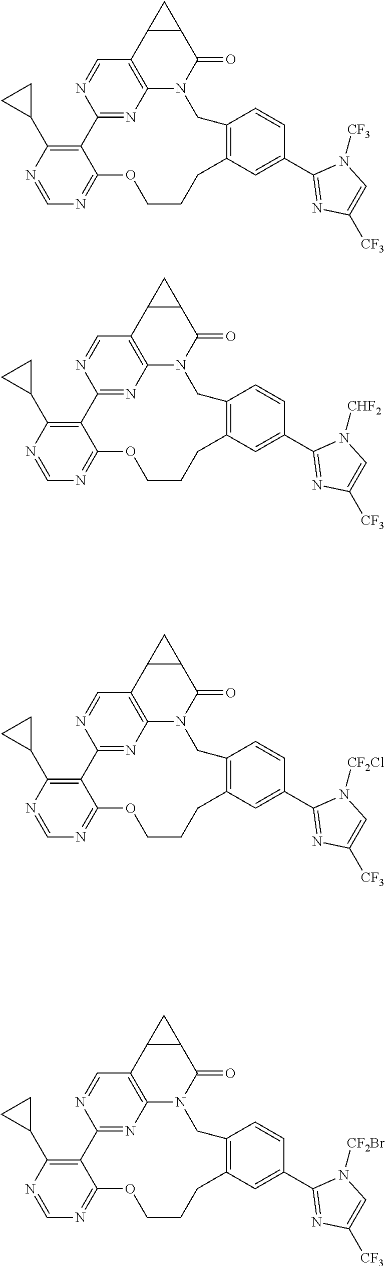

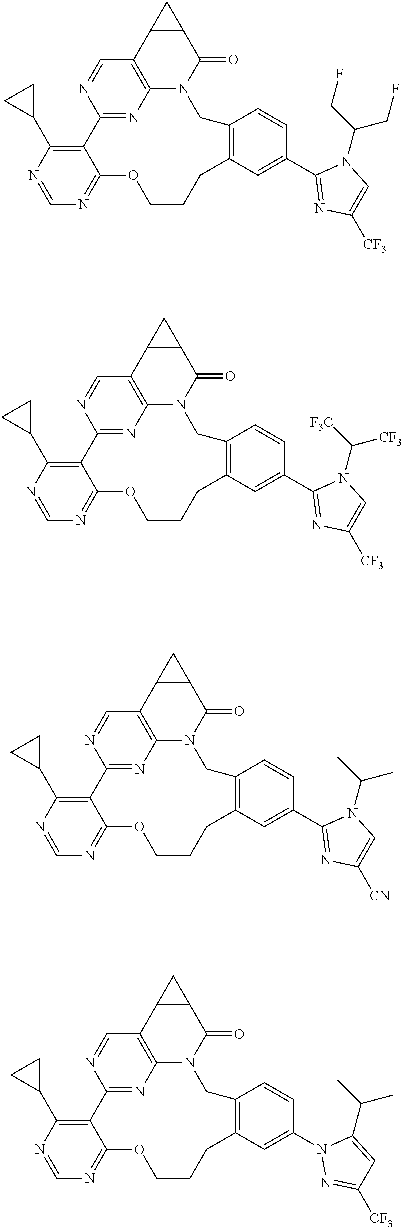 [SMILES: O=C1C2CC2c2cnc3nc2N1Cc1ccc(-c2nc(C(F)(F)F)cn2C(CF)CF)cc1CCCOc1ncnc(C2CC2)c1-3 ; O=C1C2CC2c2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)C1CC31 ; CC(C)c1cc(C(F)(F)F)nn1-c1ccc2c(c1)CCCOc1ncnc(C3CC3)c1-c1ncc3c(n1)N(C2)C(=O)C1CC31]

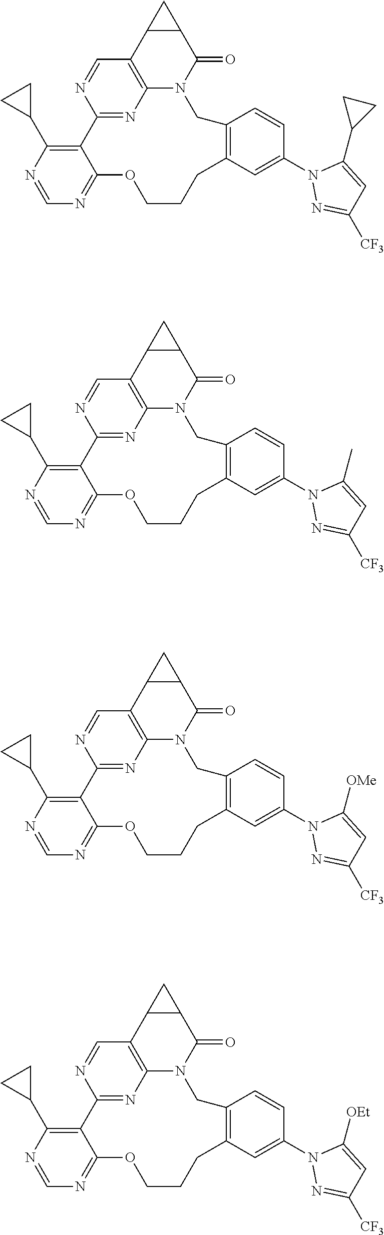 [SMILES: O=C1C2CC2c2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)C1CC31 ; COc1cc(C(F)(F)F)nn1-c1ccc2c(c1)CCCOc1ncnc(C3CC3)c1-c1ncc3c(n1)N(C2)C(=O)C1CC31 ; CCOc1cc(C(F)(F)F)nn1-c1ccc2c(c1)CCCOc1ncnc(C3CC3)c1-c1ncc3c(n1)N(C2)C(=O)C1CC31]

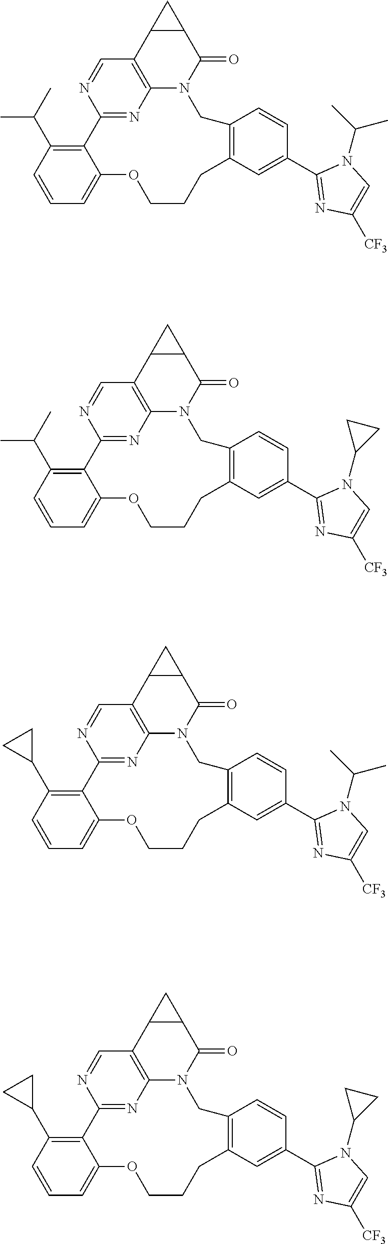

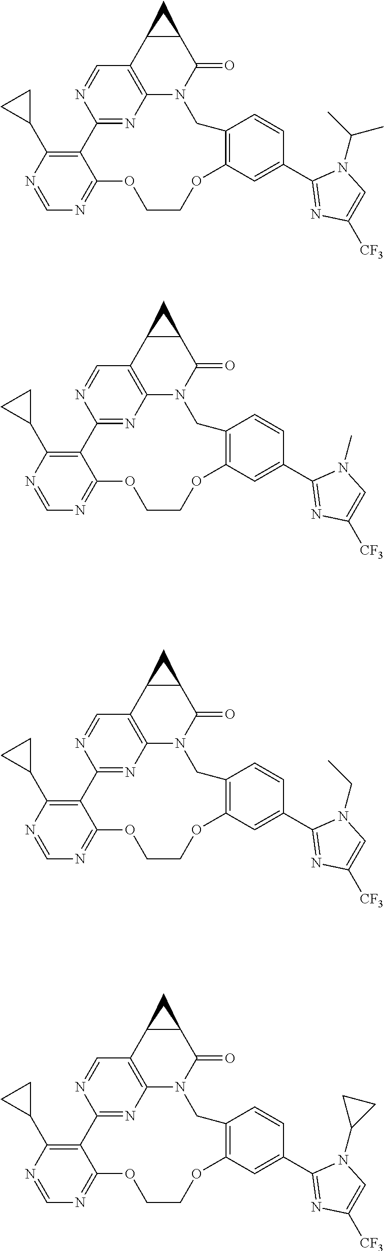 [SMILES: CC(C)n1cc(C(F)(F)F)nc1-c1ccc2c(c1)OCCOc1ncnc(C3CC3)c1-c1ncc3c(n1)N(C2)C(=O)[C@@H]1C[C@H]31 ; Cn1cc(C(F)(F)F)nc1-c1ccc2c(c1)OCCOc1ncnc(C3CC3)c1-c1ncc3c(n1)N(C2)C(=O)[C@@H]1C[C@H]31 ; CCn1cc(C(F)(F)F)nc1-c1ccc2c(c1)OCCOc1ncnc(C3CC3)c1-c1ncc3c(n1)N(C2)C(=O)[C@@H]1C[C@H]31 ; O=C1[C@@H]2C[C@@H]2c2cnc3nc2N1Cc1ccc(-c2nc(C(F)(F)F)cn2C2CC2)cc1OCCOc1ncnc(C2CC2)c1-3]

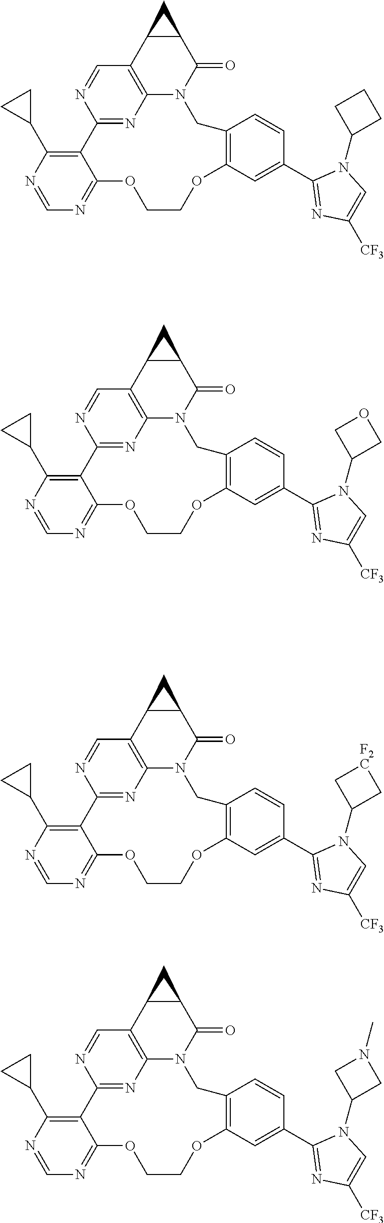 [SMILES: O=C1[C@@H]2C[C@@H]2c2cnc3nc2N1Cc1ccc(-c2nc(C(F)(F)F)cn2C2CCC2)cc1OCCOc1ncnc(C2CC2)c1-3 ; O=C1[C@@H]2C[C@@H]2c2cnc3nc2N1Cc1ccc(-c2nc(C(F)(F)F)cn2C2COC2)cc1OCCOc1ncnc(C2CC2)c1-3 ; O=C1[C@@H]2C[C@@H]2c2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)[C@@H]2C[C@H]42)C1]

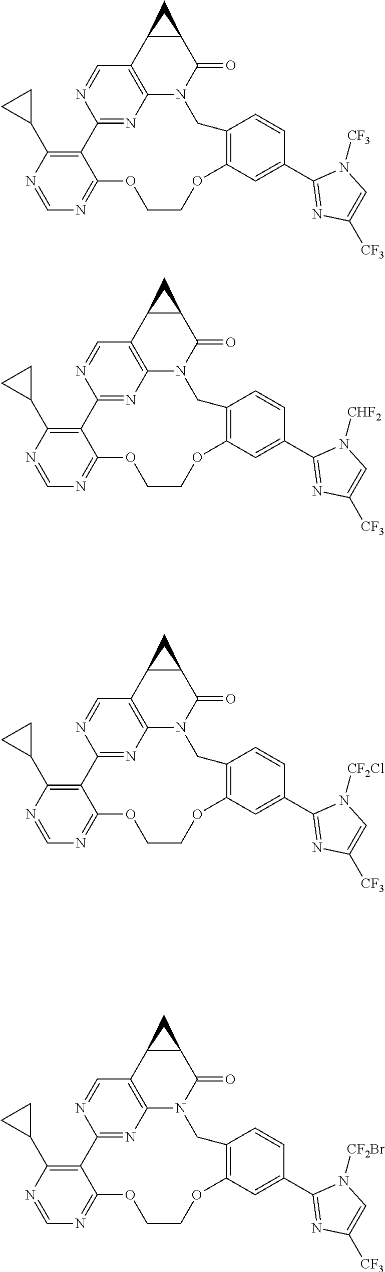

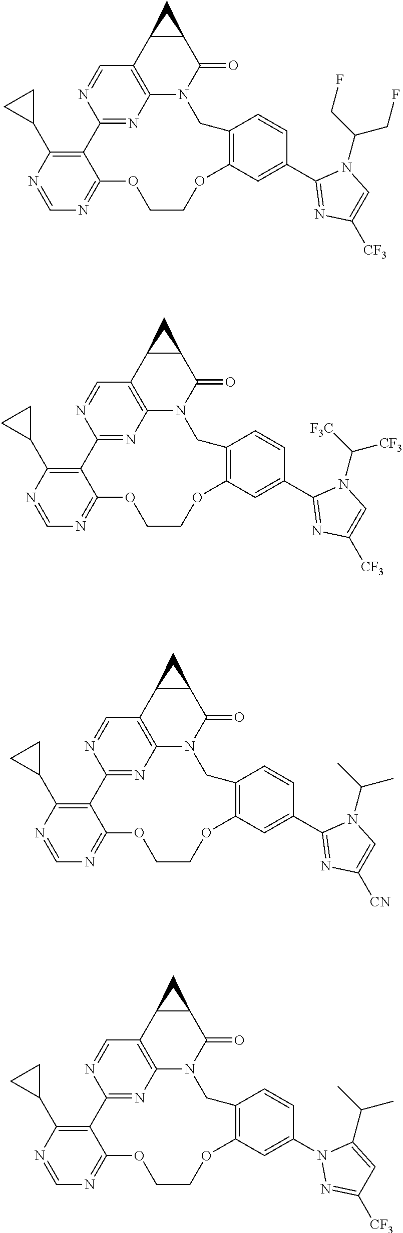 [SMILES: O=C1[C@@H]2C[C@@H]2c2cnc3nc2N1Cc1ccc(-c2nc(C(F)(F)F)cn2C(CF)CF)cc1OCCOc1ncnc(C2CC2)c1-3 ; O=C1[C@@H]2C[C@@H]2c2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)[C@@H]1C[C@H]31 ; CC(C)c1cc(C(F)(F)F)nn1-c1ccc2c(c1)OCCOc1ncnc(C3CC3)c1-c1ncc3c(n1)N(C2)C(=O)[C@@H]1C[C@H]31]

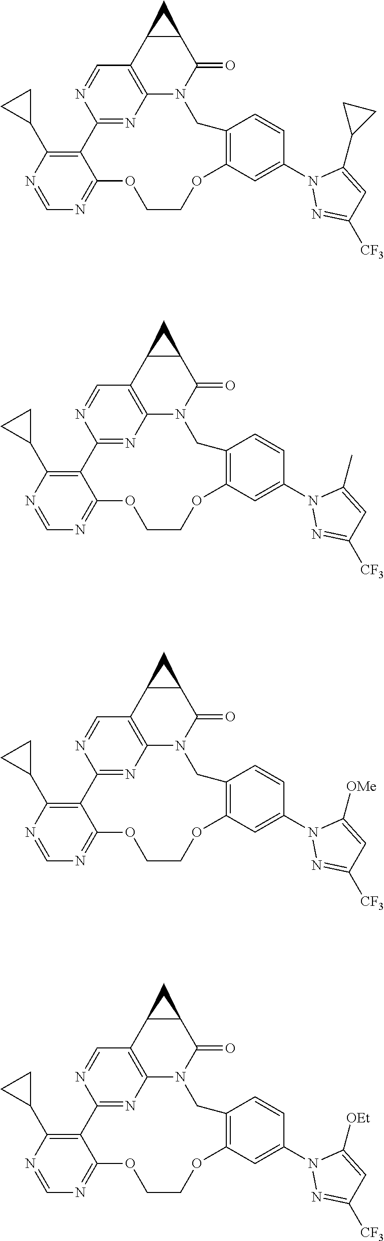 [SMILES: O=C1[C@@H]2C[C@@H]2c2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)[C@@H]1C[C@H]31 ; COc1cc(C(F)(F)F)nn1-c1ccc2c(c1)OCCOc1ncnc(C3CC3)c1-c1ncc3c(n1)N(C2)C(=O)[C@@H]1C[C@H]31 ; CCOc1cc(C(F)(F)F)nn1-c1ccc2c(c1)OCCOc1ncnc(C3CC3)c1-c1ncc3c(n1)N(C2)C(=O)[C@@H]1C[C@H]31]

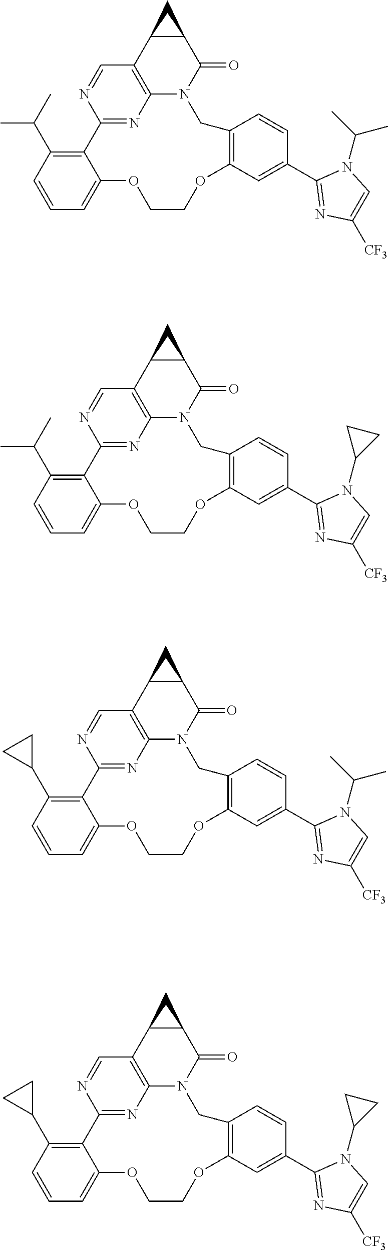

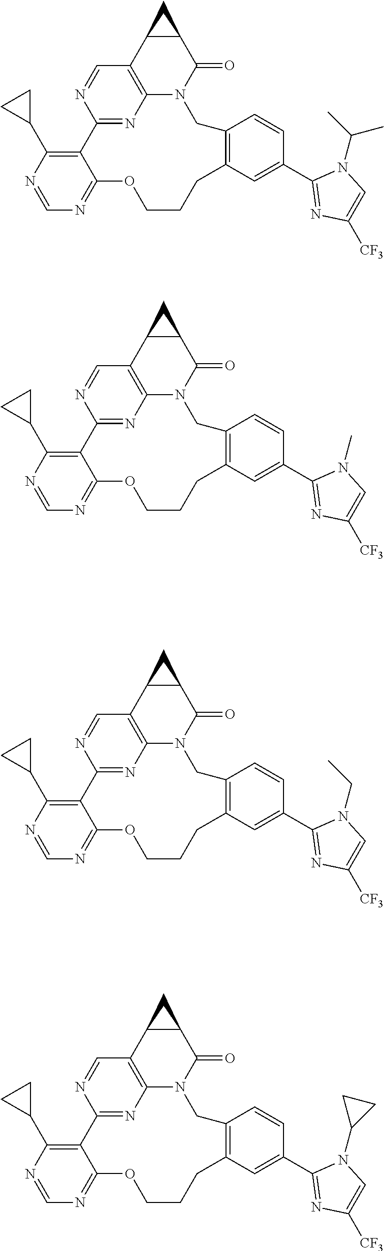 [SMILES: CC(C)n1cc(C(F)(F)F)nc1-c1ccc2c(c1)CCCOc1ncnc(C3CC3)c1-c1ncc3c(n1)N(C2)C(=O)[C@@H]1C[C@H]31 ; Cn1cc(C(F)(F)F)nc1-c1ccc2c(c1)CCCOc1ncnc(C3CC3)c1-c1ncc3c(n1)N(C2)C(=O)[C@@H]1C[C@H]31 ; CCn1cc(C(F)(F)F)nc1-c1ccc2c(c1)CCCOc1ncnc(C3CC3)c1-c1ncc3c(n1)N(C2)C(=O)[C@@H]1C[C@H]31 ; O=C1[C@@H]2C[C@@H]2c2cnc3nc2N1Cc1ccc(-c2nc(C(F)(F)F)cn2C2CC2)cc1CCCOc1ncnc(C2CC2)c1-3]

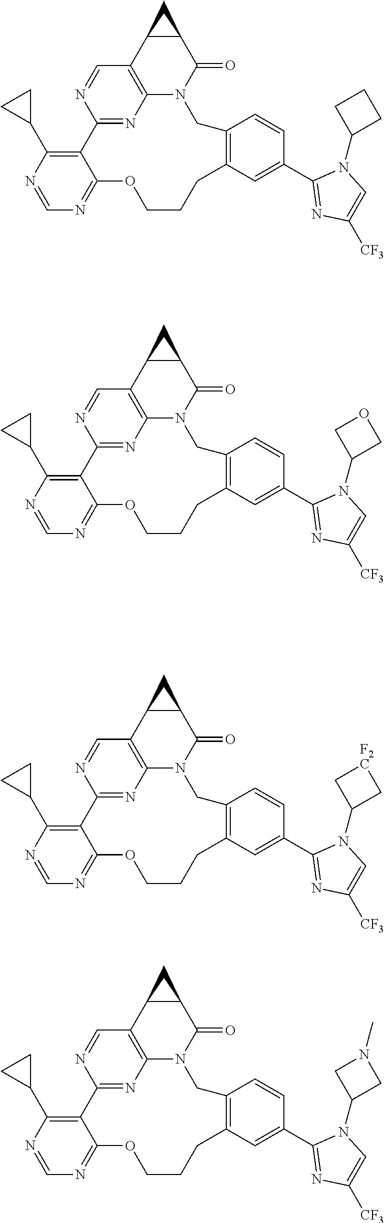 [SMILES: O=C1[C@@H]2C[C@@H]2c2cnc3nc2N1Cc1ccc(-c2nc(C(F)(F)F)cn2C2CCC2)cc1CCCOc1ncnc(C2CC2)c1-3 ; O=C1[C@@H]2C[C@@H]2c2cnc3nc2N1Cc1ccc(-c2nc(C(F)(F)F)cn2C2COC2)cc1CCCOc1ncnc(C2CC2)c1-3 ; O=C1[C@@H]2C[C@@H]2c2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)[C@@H]2C[C@H]42)C1]

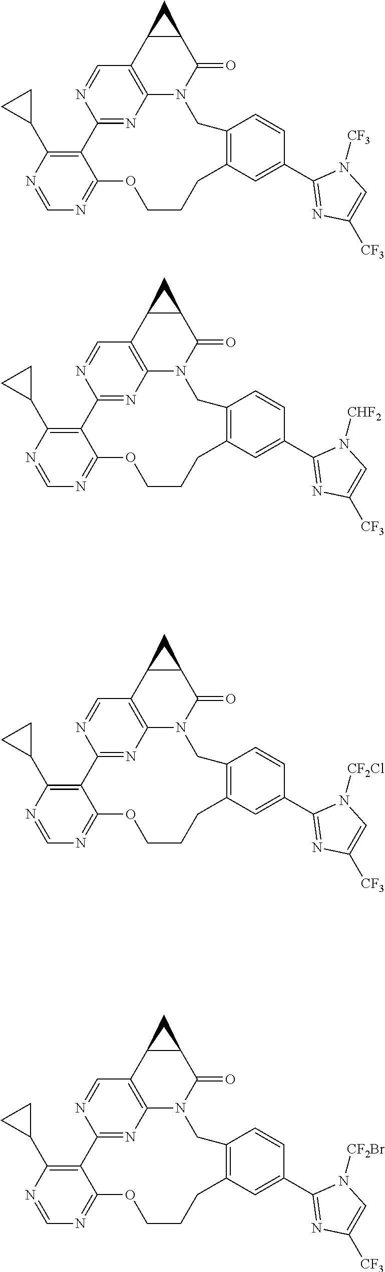

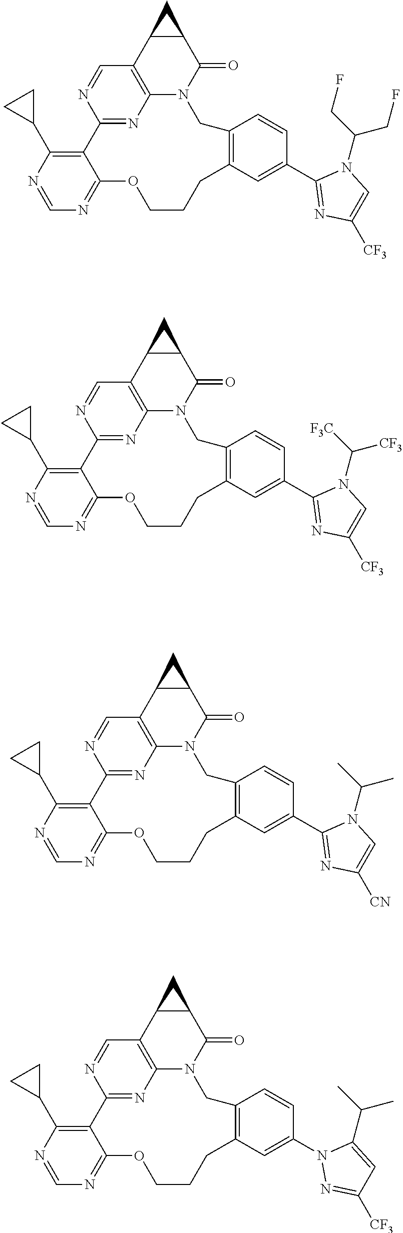 [SMILES: O=C1[C@@H]2C[C@@H]2c2cnc3nc2N1Cc1ccc(-c2nc(C(F)(F)F)cn2C(CF)CF)cc1CCCOc1ncnc(C2CC2)c1-3 ; O=C1[C@@H]2C[C@@H]2c2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)[C@@H]1C[C@H]31 ; CC(C)c1cc(C(F)(F)F)nn1-c1ccc2c(c1)CCCOc1ncnc(C3CC3)c1-c1ncc3c(n1)N(C2)C(=O)[C@@H]1C[C@H]31]

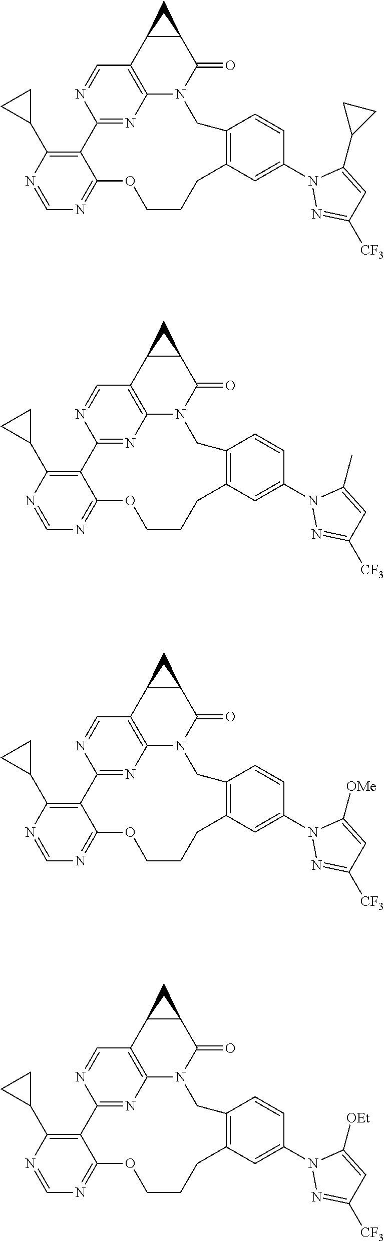 [SMILES: O=C1[C@@H]2C[C@@H]2c2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)[C@@H]1C[C@H]31 ; COc1cc(C(F)(F)F)nn1-c1ccc2c(c1)CCCOc1ncnc(C3CC3)c1-c1ncc3c(n1)N(C2)C(=O)[C@@H]1C[C@H]31 ; CCOc1cc(C(F)(F)F)nn1-c1ccc2c(c1)CCCOc1ncnc(C3CC3)c1-c1ncc3c(n1)N(C2)C(=O)[C@@H]1C[C@H]31]

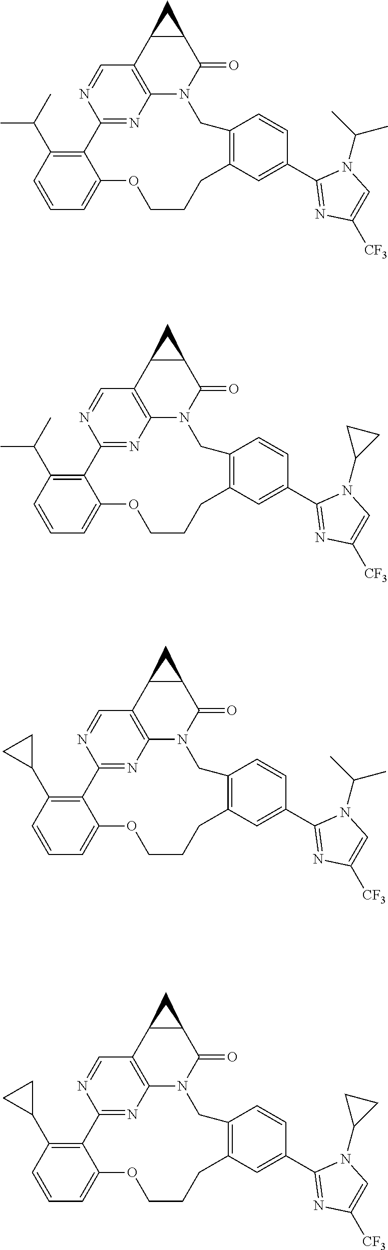

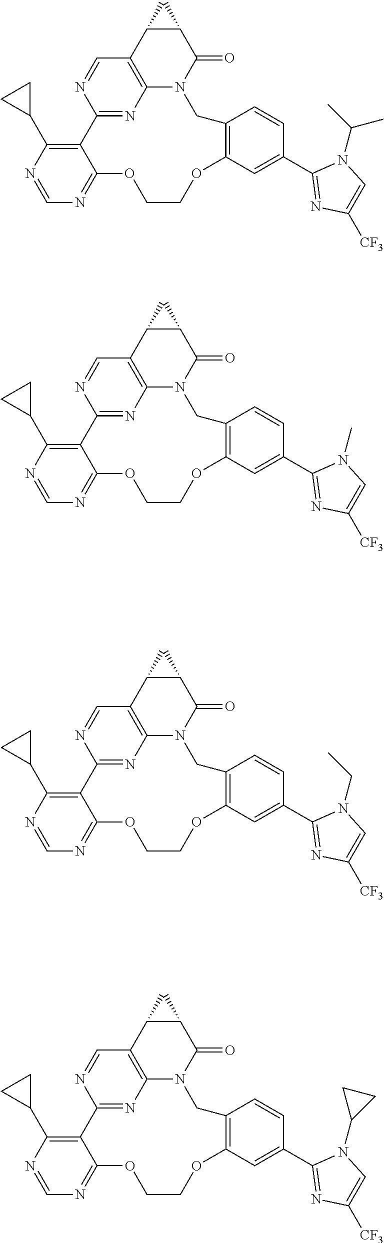 [SMILES: CC(C)n1cc(C(F)(F)F)nc1-c1ccc2c(c1)OCCOc1ncnc(C3CC3)c1-c1ncc3c(n1)N(C2)C(=O)[C@H]1C[C@@H]31 ; Cn1cc(C(F)(F)F)nc1-c1ccc2c(c1)OCCOc1ncnc(C3CC3)c1-c1ncc3c(n1)N(C2)C(=O)[C@H]1C[C@@H]31 ; CCn1cc(C(F)(F)F)nc1-c1ccc2c(c1)OCCOc1ncnc(C3CC3)c1-c1ncc3c(n1)N(C2)C(=O)[C@H]1C[C@@H]31 ; O=C1[C@H]2C[C@H]2c2cnc3nc2N1Cc1ccc(-c2nc(C(F)(F)F)cn2C2CC2)cc1OCCOc1ncnc(C2CC2)c1-3]

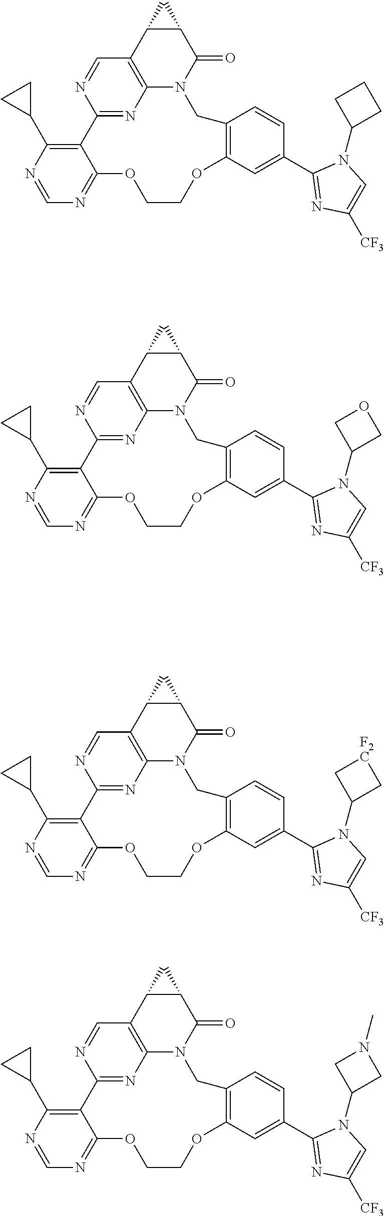 [SMILES: O=C1[C@H]2C[C@H]2c2cnc3nc2N1Cc1ccc(-c2nc(C(F)(F)F)cn2C2CCC2)cc1OCCOc1ncnc(C2CC2)c1-3 ; O=C1[C@H]2C[C@H]2c2cnc3nc2N1Cc1ccc(-c2nc(C(F)(F)F)cn2C2COC2)cc1OCCOc1ncnc(C2CC2)c1-3 ; O=C1[C@H]2C[C@H]2c2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)[C@H]2C[C@@H]42)C1]

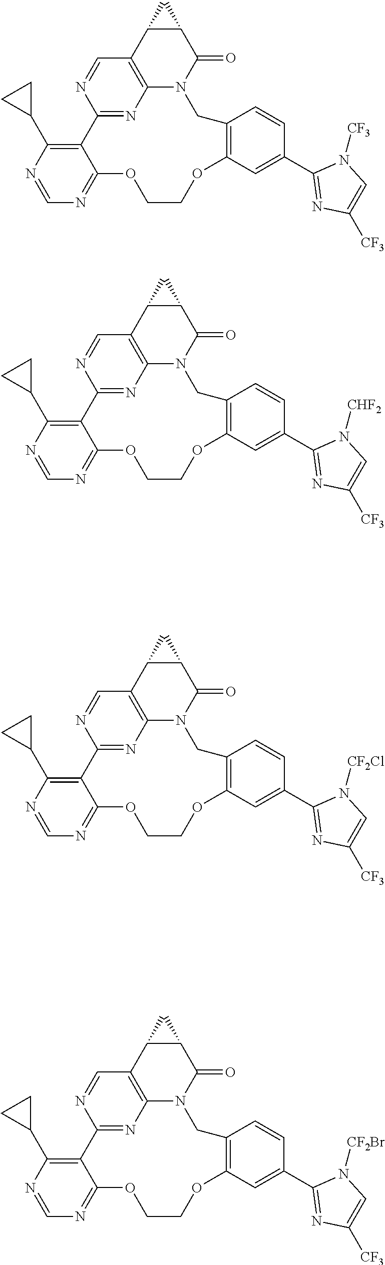

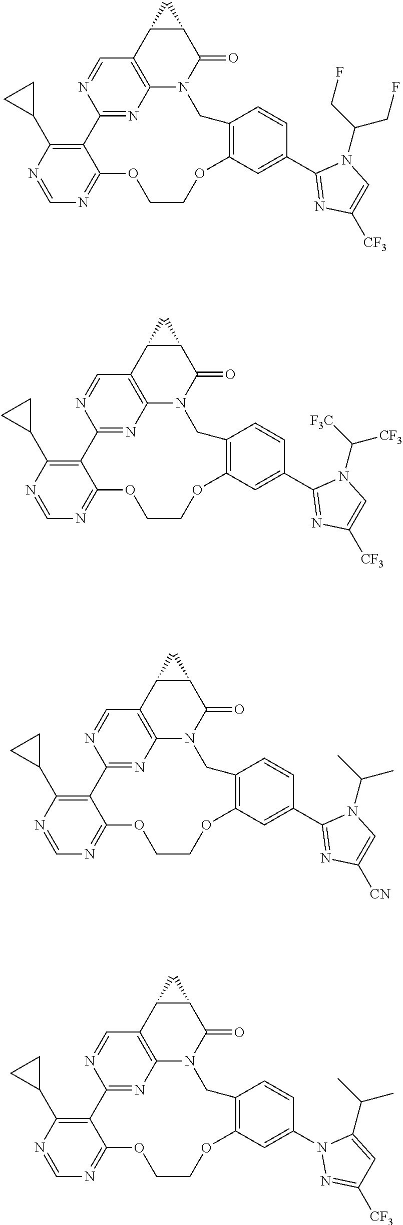 [SMILES: O=C1[C@H]2C[C@H]2c2cnc3nc2N1Cc1ccc(-c2nc(C(F)(F)F)cn2C(CF)CF)cc1OCCOc1ncnc(C2CC2)c1-3 ; O=C1[C@H]2C[C@H]2c2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)[C@H]1C[C@@H]31 ; CC(C)c1cc(C(F)(F)F)nn1-c1ccc2c(c1)OCCOc1ncnc(C3CC3)c1-c1ncc3c(n1)N(C2)C(=O)[C@H]1C[C@@H]31]

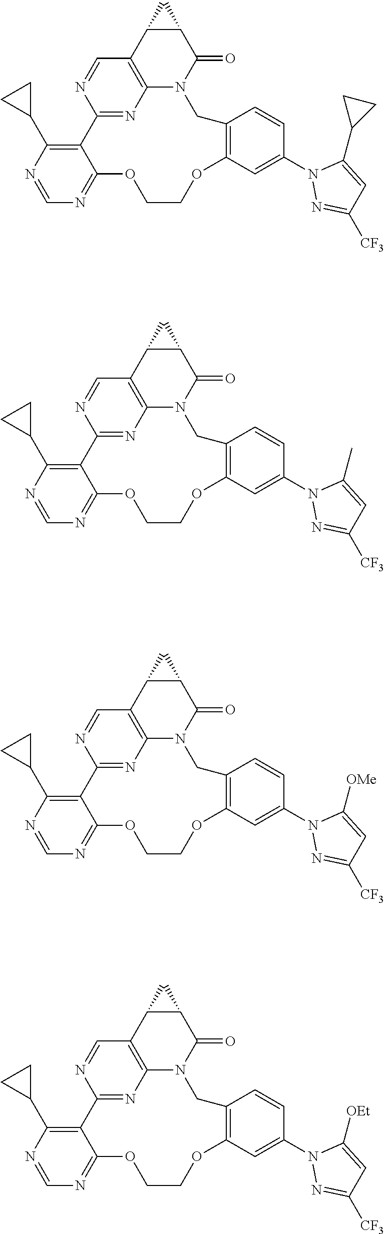 [SMILES: O=C1[C@H]2C[C@H]2c2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)[C@H]1C[C@@H]31 ; COc1cc(C(F)(F)F)nn1-c1ccc2c(c1)OCCOc1ncnc(C3CC3)c1-c1ncc3c(n1)N(C2)C(=O)[C@H]1C[C@@H]31 ; CCOc1cc(C(F)(F)F)nn1-c1ccc2c(c1)OCCOc1ncnc(C3CC3)c1-c1ncc3c(n1)N(C2)C(=O)[C@H]1C[C@@H]31]

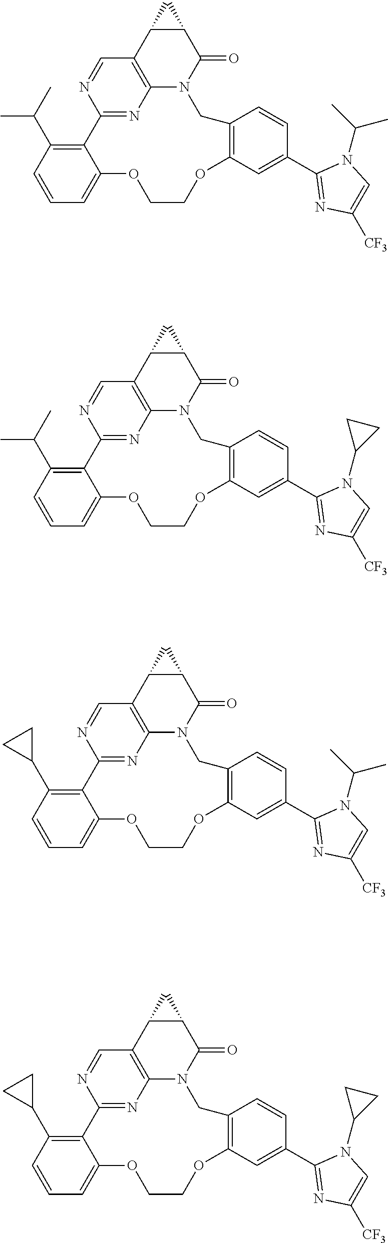

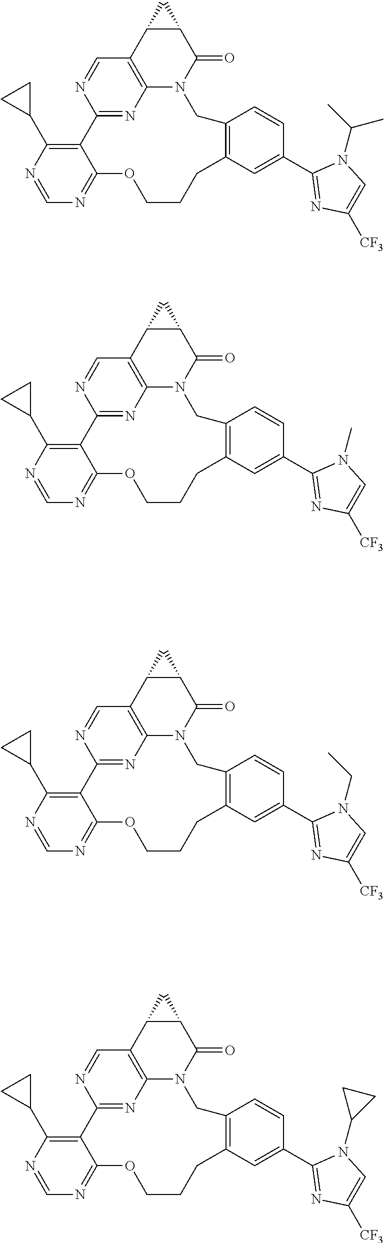 [SMILES: CC(C)n1cc(C(F)(F)F)nc1-c1ccc2c(c1)CCCOc1ncnc(C3CC3)c1-c1ncc3c(n1)N(C2)C(=O)[C@H]1C[C@@H]31 ; Cn1cc(C(F)(F)F)nc1-c1ccc2c(c1)CCCOc1ncnc(C3CC3)c1-c1ncc3c(n1)N(C2)C(=O)[C@H]1C[C@@H]31 ; CCn1cc(C(F)(F)F)nc1-c1ccc2c(c1)CCCOc1ncnc(C3CC3)c1-c1ncc3c(n1)N(C2)C(=O)[C@H]1C[C@@H]31 ; O=C1[C@H]2C[C@H]2c2cnc3nc2N1Cc1ccc(-c2nc(C(F)(F)F)cn2C2CC2)cc1CCCOc1ncnc(C2CC2)c1-3]

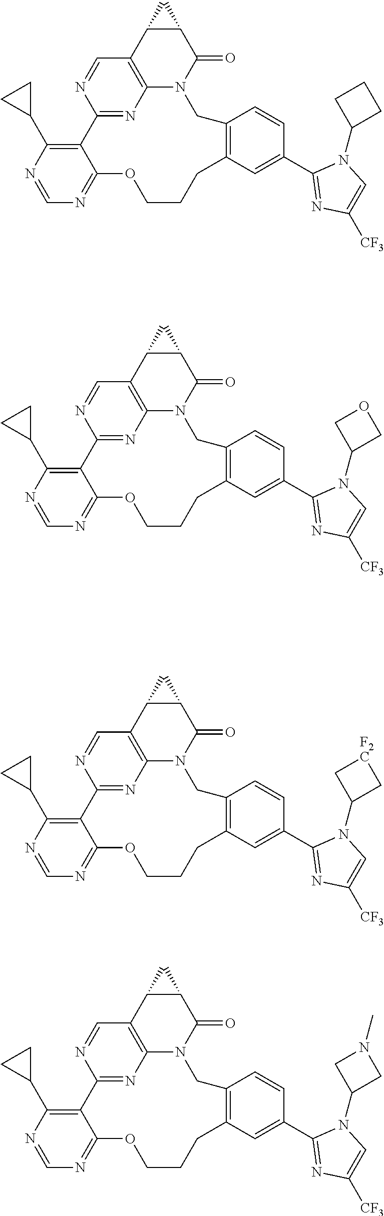 [SMILES: O=C1[C@H]2C[C@H]2c2cnc3nc2N1Cc1ccc(-c2nc(C(F)(F)F)cn2C2CCC2)cc1CCCOc1ncnc(C2CC2)c1-3 ; O=C1[C@H]2C[C@H]2c2cnc3nc2N1Cc1ccc(-c2nc(C(F)(F)F)cn2C2COC2)cc1CCCOc1ncnc(C2CC2)c1-3 ; O=C1[C@H]2C[C@H]2c2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)[C@H]2C[C@@H]42)C1]

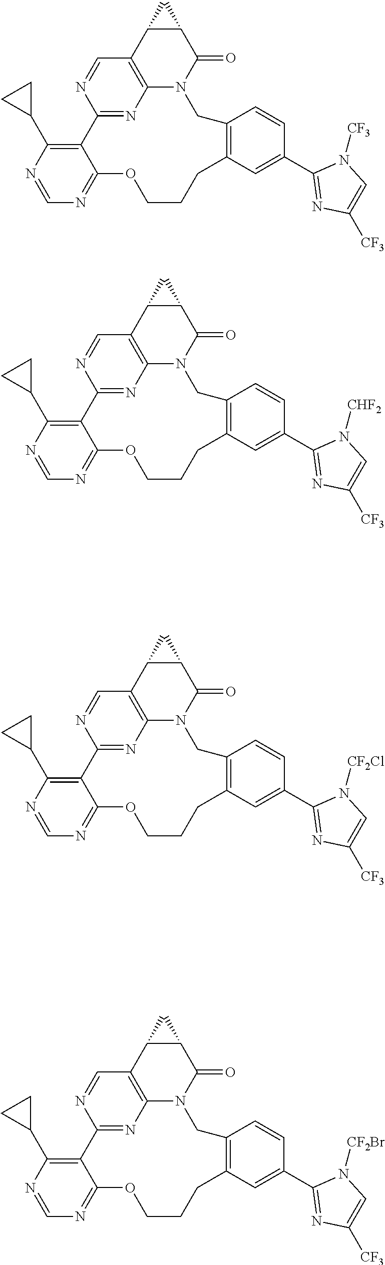

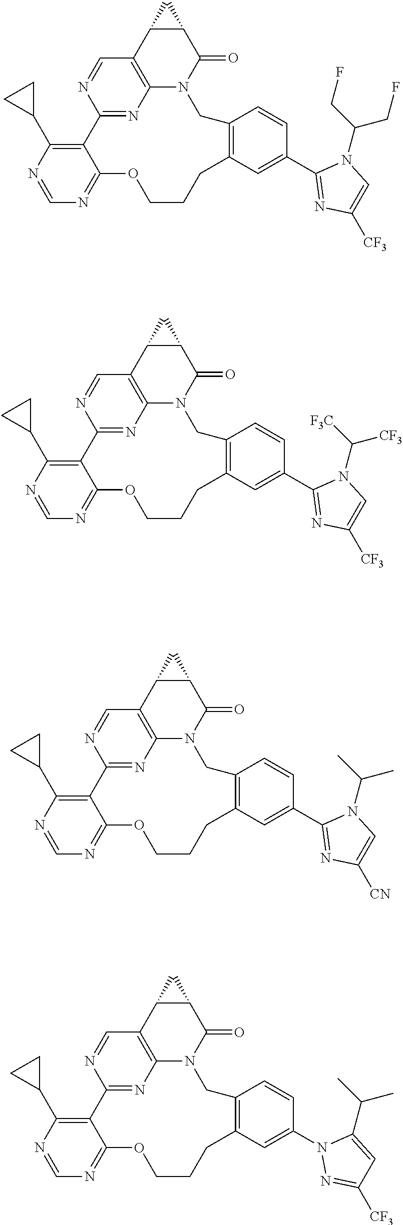 [SMILES: O=C1[C@H]2C[C@H]2c2cnc3nc2N1Cc1ccc(-c2nc(C(F)(F)F)cn2C(CF)CF)cc1CCCOc1ncnc(C2CC2)c1-3 ; O=C1[C@H]2C[C@H]2c2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)[C@H]1C[C@@H]31 ; CC(C)c1cc(C(F)(F)F)nn1-c1ccc2c(c1)CCCOc1ncnc(C3CC3)c1-c1ncc3c(n1)N(C2)C(=O)[C@H]1C[C@@H]31]

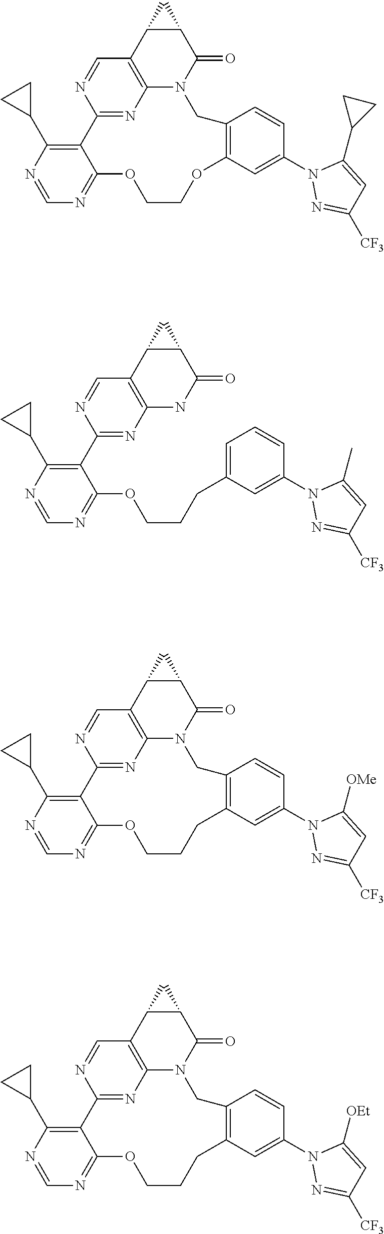 [SMILES: O=C1[C@H]2C[C@H]2c2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1cccc(CCCOc2ncnc(C3CC3)c2-c2ncc3c(n2)NC(=O)[C@H]2C[C@@H]32)c1 ; COc1cc(C(F)(F)F)nn1-c1ccc2c(c1)CCCOc1ncnc(C3CC3)c1-c1ncc3c(n1)N(C2)C(=O)[C@H]1C[C@@H]31 ; CCOc1cc(C(F)(F)F)nn1-c1ccc2c(c1)CCCOc1ncnc(C3CC3)c1-c1ncc3c(n1)N(C2)C(=O)[C@H]1C[C@@H]31]

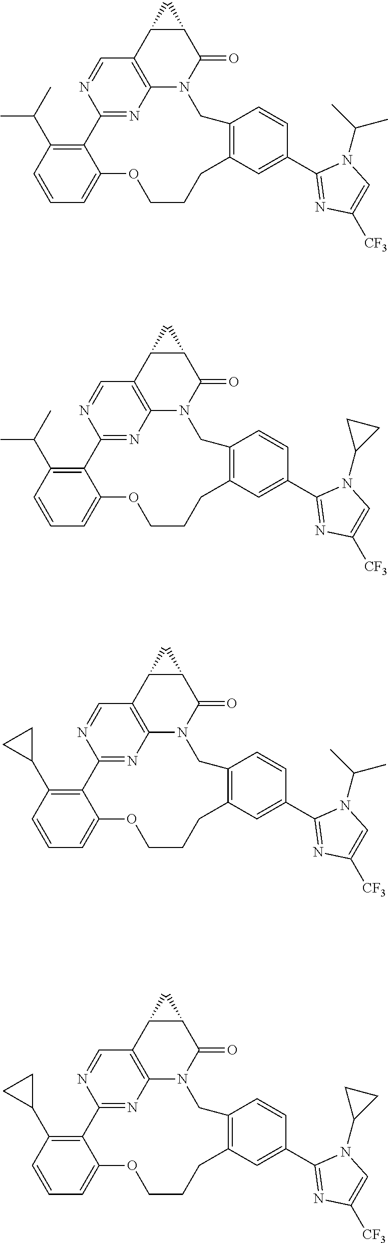

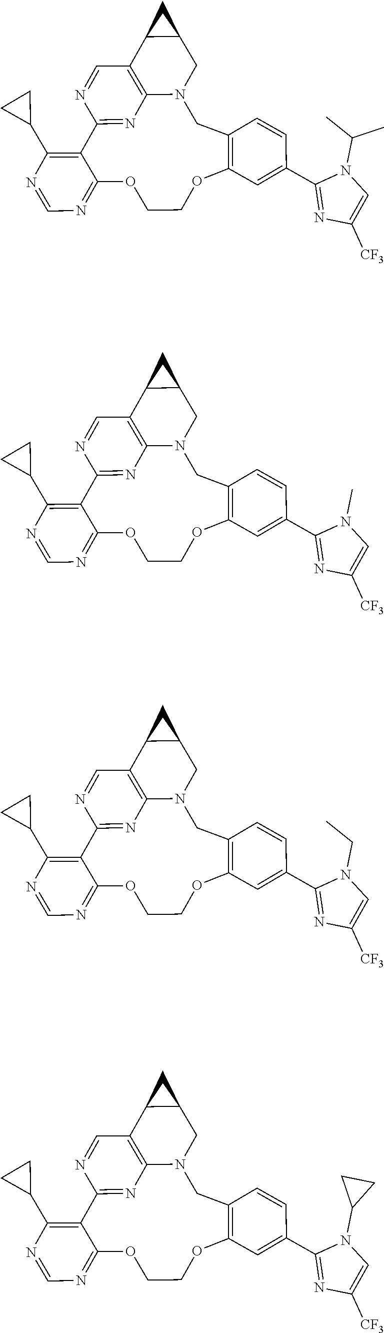 [SMILES: CC(C)n1cc(C(F)(F)F)nc1-c1ccc2c(c1)OCCOc1ncnc(C3CC3)c1-c1ncc3c(n1)N(C2)C[C@@H]1C[C@H]31 ; Cn1cc(C(F)(F)F)nc1-c1ccc2c(c1)OCCOc1ncnc(C3CC3)c1-c1ncc3c(n1)N(C2)C[C@@H]1C[C@H]31 ; CCn1cc(C(F)(F)F)nc1-c1ccc2c(c1)OCCOc1ncnc(C3CC3)c1-c1ncc3c(n1)N(C2)C[C@@H]1C[C@H]31 ; FC(F)(F)c1cn(C2CC2)c(-c2ccc3c(c2)OCCOc2ncnc(C4CC4)c2-c2ncc4c(n2)N(C3)C[C@@H]2C[C@H]42)n1]

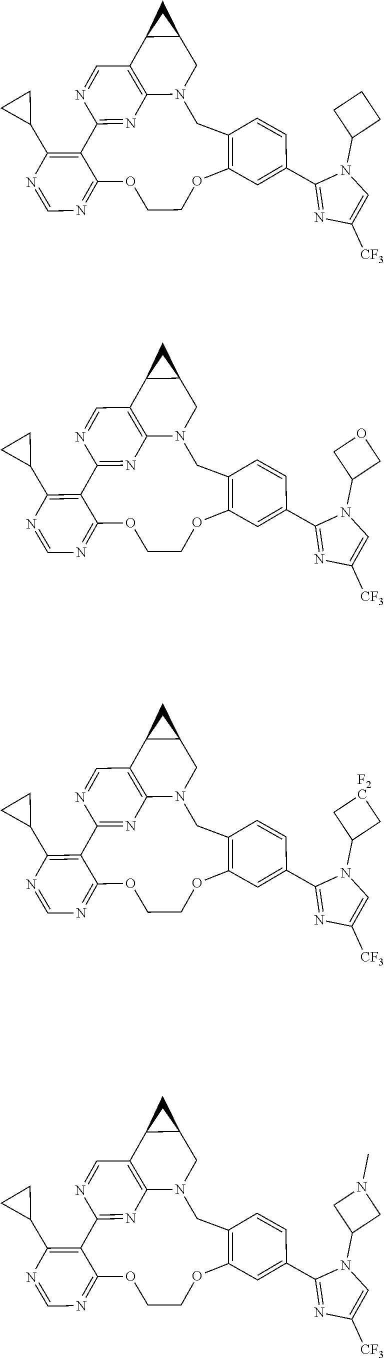 [SMILES: FC(F)(F)c1cn(C2CCC2)c(-c2ccc3c(c2)OCCOc2ncnc(C4CC4)c2-c2ncc4c(n2)N(C3)C[C@@H]2C[C@H]42)n1 ; FC(F)(F)c1cn(C2COC2)c(-c2ccc3c(c2)OCCOc2ncnc(C4CC4)c2-c2ncc4c(n2)N(C3)C[C@@H]2C[C@H]42)n1 ; FC1(F)CC(n2cc(C(F)(F)F)nc2-c2ccc3c(c2)OCCOc2ncnc(C4CC4)c2-c2ncc4c(n2)N(C3)C[C@@H]2C[C@H]42)C1 ; CN1CC(n2cc(C(F)(F)F)nc2-c2ccc3c(c2)OCCOc2ncnc(C4CC4)c2-c2ncc4c(n2)N(C3)C[C@@H]2C[C@H]42)C1]

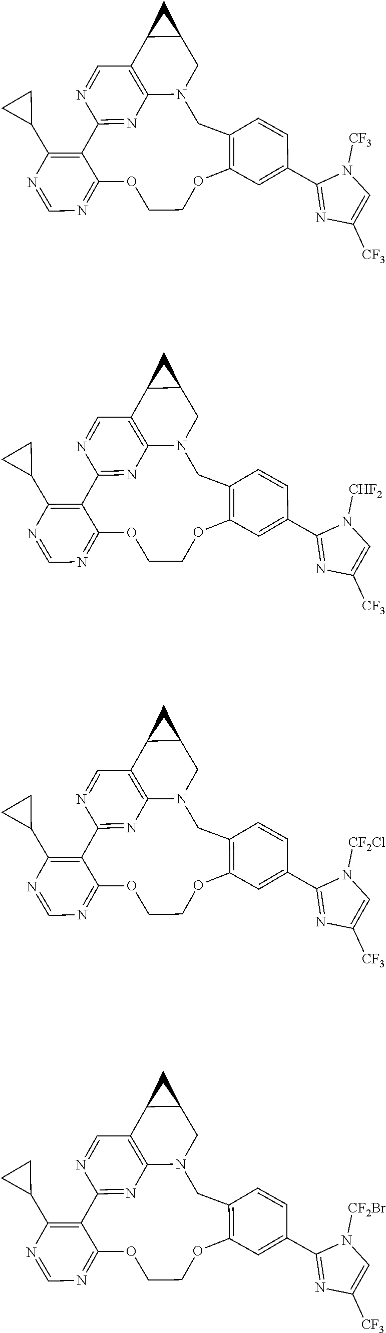

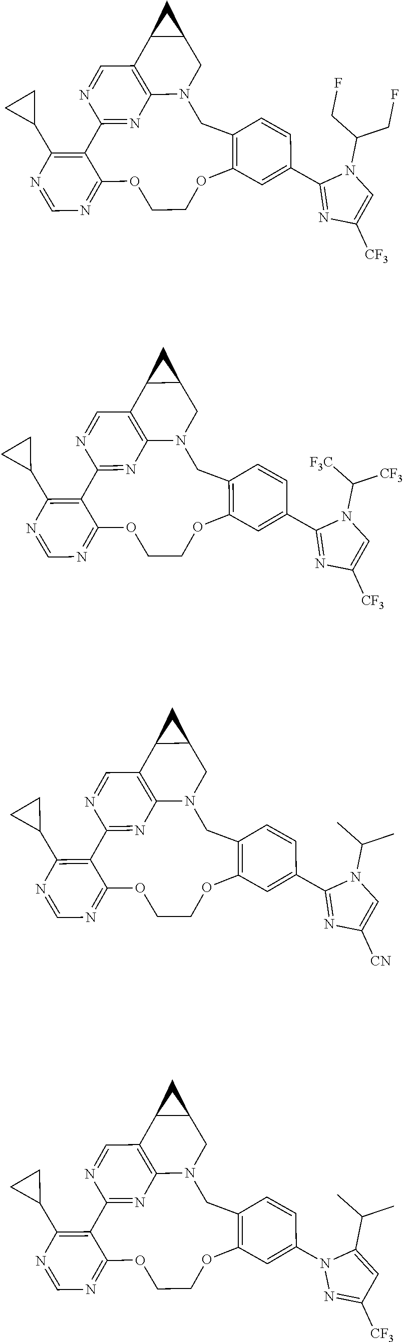 [SMILES: FCC(CF)n1cc(C(F)(F)F)nc1-c1ccc2c(c1)OCCOc1ncnc(C3CC3)c1-c1ncc3c(n1)N(C2)C[C@@H]1C[C@H]31 ; FC(F)(F)c1cn(C(C(F)(F)F)C(F)(F)F)c(-c2ccc3c(c2)OCCOc2ncnc(C4CC4)c2-c2ncc4c(n2)N(C3)C[C@@H]2C[C@H]42)n1 ; CC(C)n1cc(C#N)nc1-c1ccc2c(c1)OCCOc1ncnc(C3CC3)c1-c1ncc3c(n1)N(C2)C[C@@H]1C[C@H]31 ; CC(C)c1cc(C(F)(F)F)nn1-c1ccc2c(c1)OCCOc1ncnc(C3CC3)c1-c1ncc3c(n1)N(C2)C[C@@H]1C[C@H]31]

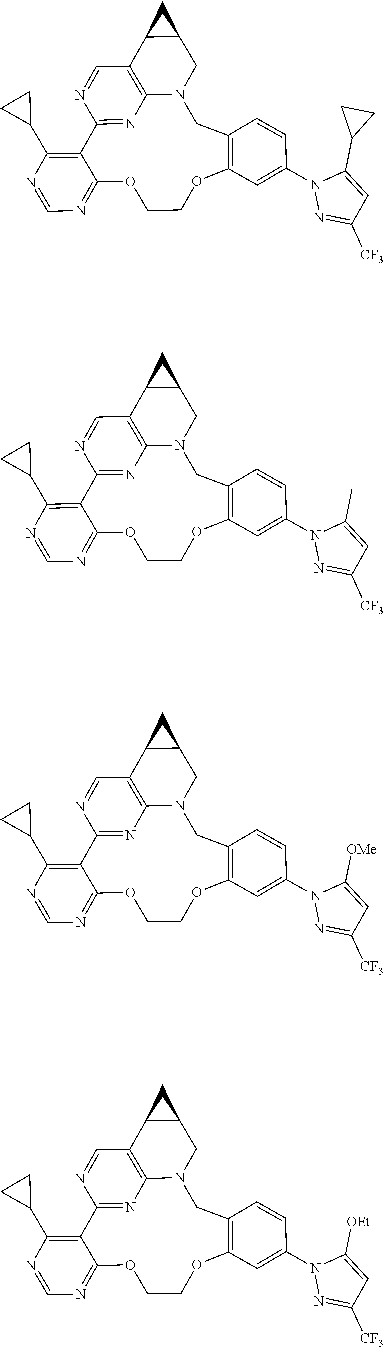 [SMILES: FC(F)(F)c1cc(C2CC2)n(-c2ccc3c(c2)OCCOc2ncnc(C4CC4)c2-c2ncc4c(n2)N(C3)C[C@@H]2C[C@H]42)n1 ; Cc1cc(C(F)(F)F)nn1-c1ccc2c(c1)OCCOc1ncnc(C3CC3)c1-c1ncc3c(n1)N(C2)C[C@@H]1C[C@H]31 ; COc1cc(C(F)(F)F)nn1-c1ccc2c(c1)OCCOc1ncnc(C3CC3)c1-c1ncc3c(n1)N(C2)C[C@@H]1C[C@H]31 ; CCOc1cc(C(F)(F)F)nn1-c1ccc2c(c1)OCCOc1ncnc(C3CC3)c1-c1ncc3c(n1)N(C2)C[C@@H]1C[C@H]31]

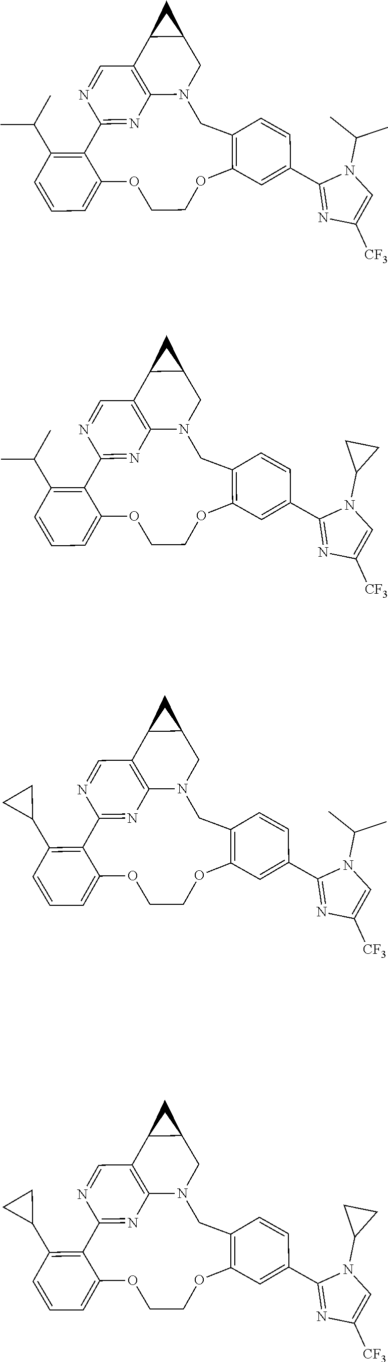

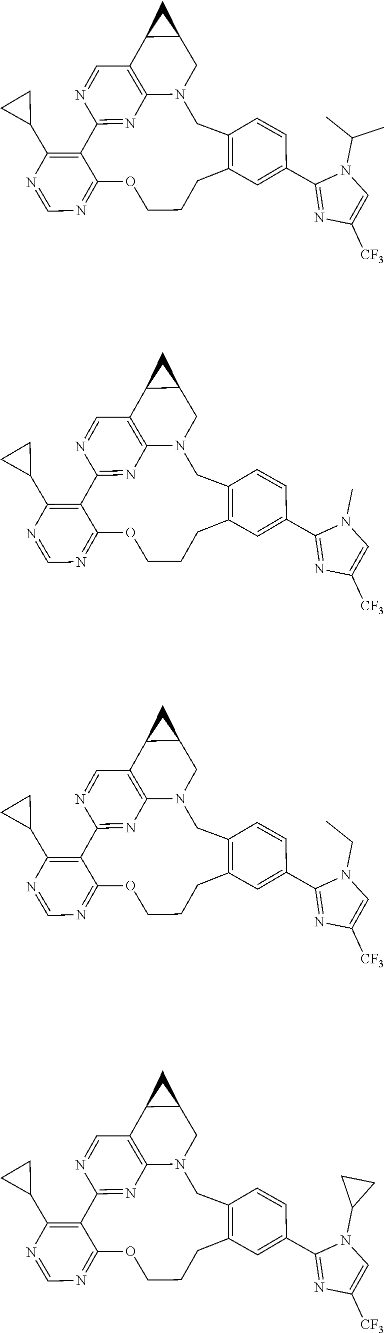 [SMILES: CC(C)n1cc(C(F)(F)F)nc1-c1ccc2c(c1)CCCOc1ncnc(C3CC3)c1-c1ncc3c(n1)N(C2)C[C@@H]1C[C@H]31 ; Cn1cc(C(F)(F)F)nc1-c1ccc2c(c1)CCCOc1ncnc(C3CC3)c1-c1ncc3c(n1)N(C2)C[C@@H]1C[C@H]31 ; CCn1cc(C(F)(F)F)nc1-c1ccc2c(c1)CCCOc1ncnc(C3CC3)c1-c1ncc3c(n1)N(C2)C[C@@H]1C[C@H]31 ; FC(F)(F)c1cn(C2CC2)c(-c2ccc3c(c2)CCCOc2ncnc(C4CC4)c2-c2ncc4c(n2)N(C3)C[C@@H]2C[C@H]42)n1]

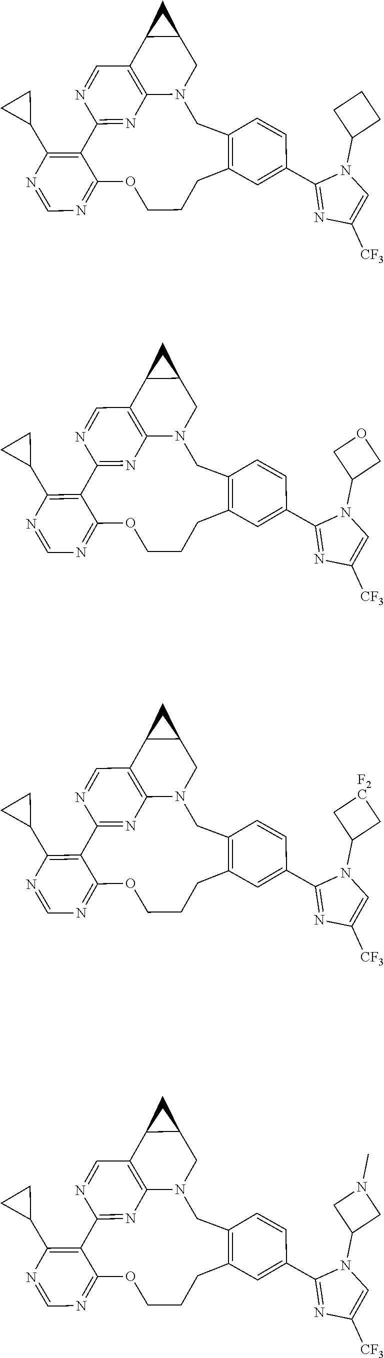 [SMILES: FC(F)(F)c1cn(C2CCC2)c(-c2ccc3c(c2)CCCOc2ncnc(C4CC4)c2-c2ncc4c(n2)N(C3)C[C@@H]2C[C@H]42)n1 ; FC(F)(F)c1cn(C2COC2)c(-c2ccc3c(c2)CCCOc2ncnc(C4CC4)c2-c2ncc4c(n2)N(C3)C[C@@H]2C[C@H]42)n1 ; FC1(F)CC(n2cc(C(F)(F)F)nc2-c2ccc3c(c2)CCCOc2ncnc(C4CC4)c2-c2ncc4c(n2)N(C3)C[C@@H]2C[C@H]42)C1 ; CN1CC(n2cc(C(F)(F)F)nc2-c2ccc3c(c2)CCCOc2ncnc(C4CC4)c2-c2ncc4c(n2)N(C3)C[C@@H]2C[C@H]42)C1]

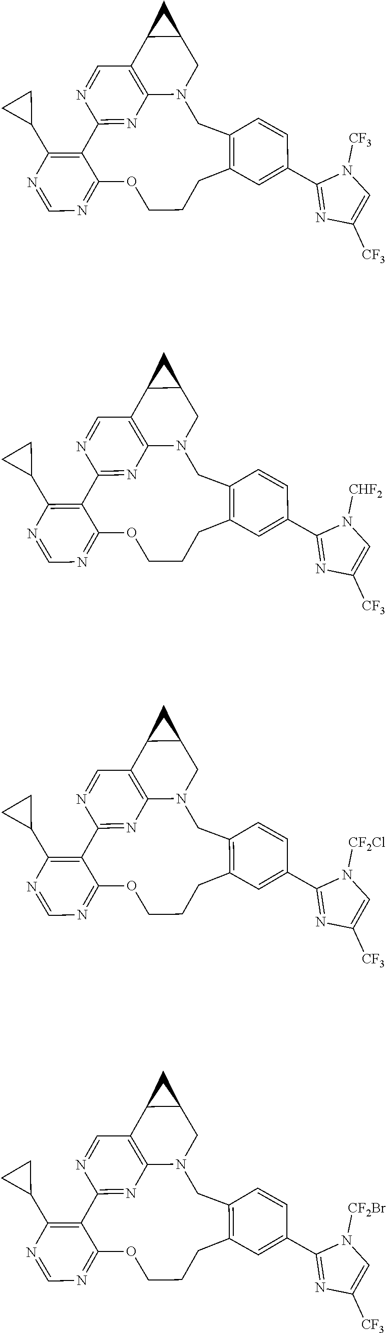

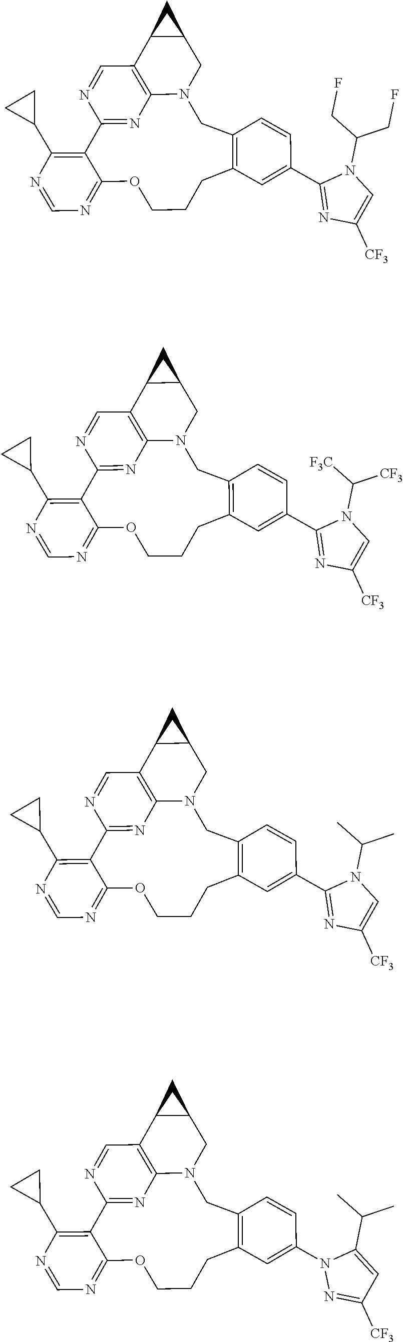 [SMILES: FCC(CF)n1cc(C(F)(F)F)nc1-c1ccc2c(c1)CCCOc1ncnc(C3CC3)c1-c1ncc3c(n1)N(C2)C[C@@H]1C[C@H]31 ; FC(F)(F)c1cn(C(C(F)(F)F)C(F)(F)F)c(-c2ccc3c(c2)CCCOc2ncnc(C4CC4)c2-c2ncc4c(n2)N(C3)C[C@@H]2C[C@H]42)n1 ; CC(C)n1cc(C(F)(F)F)nc1-c1ccc2c(c1)CCCOc1ncnc(C3CC3)c1-c1ncc3c(n1)N(C2)C[C@@H]1C[C@H]31 ; CC(C)c1cc(C(F)(F)F)nn1-c1ccc2c(c1)CCCOc1ncnc(C3CC3)c1-c1ncc3c(n1)N(C2)C[C@@H]1C[C@H]31]

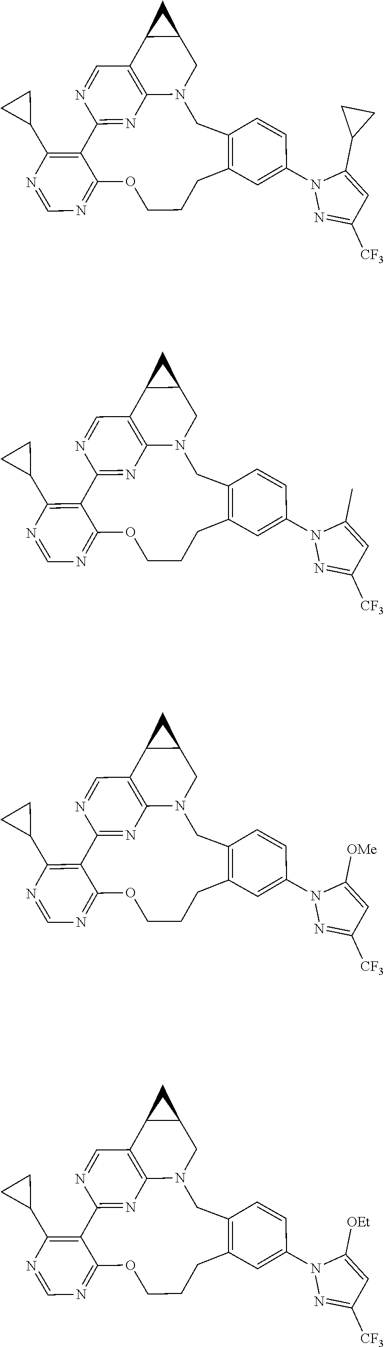 [SMILES: FC(F)(F)c1cc(C2CC2)n(-c2ccc3c(c2)CCCOc2ncnc(C4CC4)c2-c2ncc4c(n2)N(C3)C[C@@H]2C[C@H]42)n1 ; Cc1cc(C(F)(F)F)nn1-c1ccc2c(c1)CCCOc1ncnc(C3CC3)c1-c1ncc3c(n1)N(C2)C[C@@H]1C[C@H]31 ; COc1cc(C(F)(F)F)nn1-c1ccc2c(c1)CCCOc1ncnc(C3CC3)c1-c1ncc3c(n1)N(C2)C[C@@H]1C[C@H]31 ; CCOc1cc(C(F)(F)F)nn1-c1ccc2c(c1)CCCOc1ncnc(C3CC3)c1-c1ncc3c(n1)N(C2)C[C@@H]1C[C@H]31]

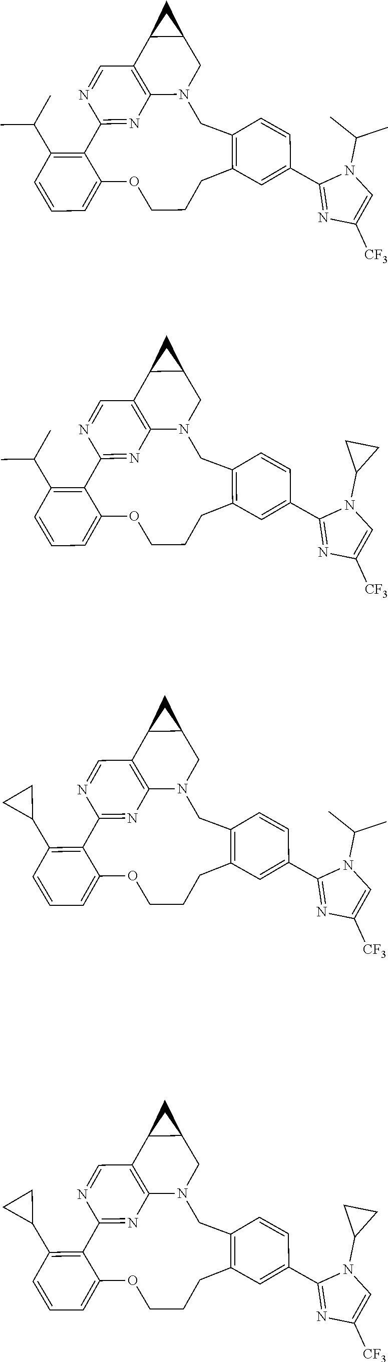

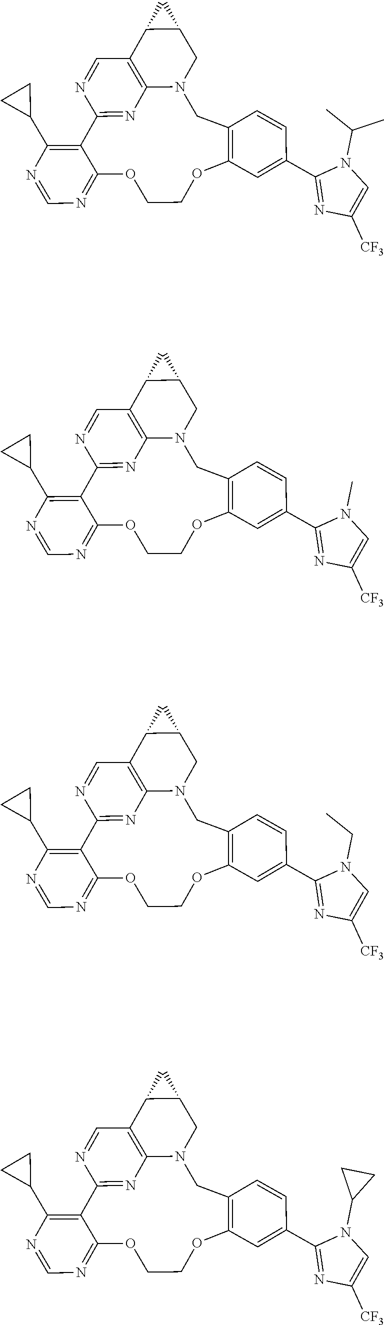 [SMILES: CC(C)n1cc(C(F)(F)F)nc1-c1ccc2c(c1)OCCOc1ncnc(C3CC3)c1-c1ncc3c(n1)N(C2)C[C@H]1C[C@@H]31 ; Cn1cc(C(F)(F)F)nc1-c1ccc2c(c1)OCCOc1ncnc(C3CC3)c1-c1ncc3c(n1)N(C2)C[C@H]1C[C@@H]31 ; CCn1cc(C(F)(F)F)nc1-c1ccc2c(c1)OCCOc1ncnc(C3CC3)c1-c1ncc3c(n1)N(C2)C[C@H]1C[C@@H]31 ; FC(F)(F)c1cn(C2CC2)c(-c2ccc3c(c2)OCCOc2ncnc(C4CC4)c2-c2ncc4c(n2)N(C3)C[C@H]2C[C@@H]42)n1]

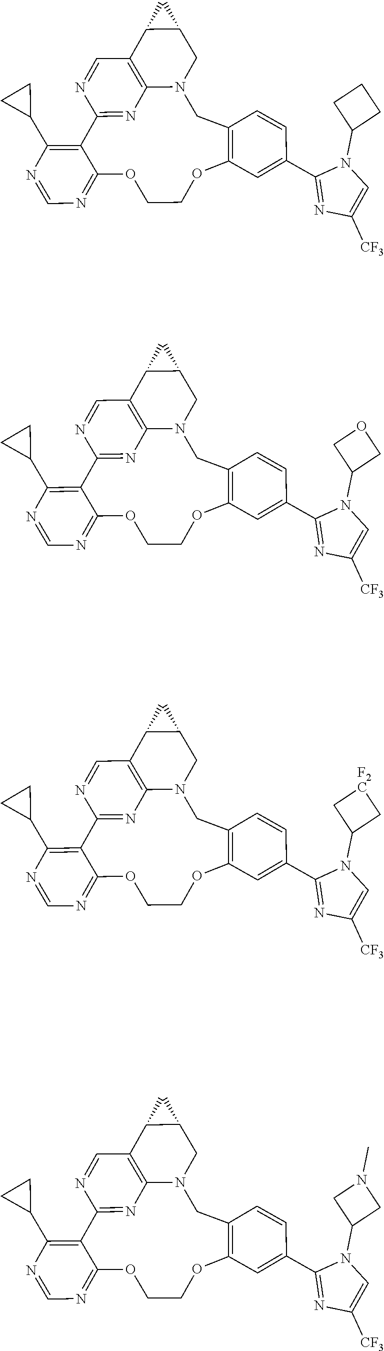 [SMILES: FC(F)(F)c1cn(C2CCC2)c(-c2ccc3c(c2)OCCOc2ncnc(C4CC4)c2-c2ncc4c(n2)N(C3)C[C@H]2C[C@@H]42)n1 ; FC(F)(F)c1cn(C2COC2)c(-c2ccc3c(c2)OCCOc2ncnc(C4CC4)c2-c2ncc4c(n2)N(C3)C[C@H]2C[C@@H]42)n1 ; FC1(F)CC(n2cc(C(F)(F)F)nc2-c2ccc3c(c2)OCCOc2ncnc(C4CC4)c2-c2ncc4c(n2)N(C3)C[C@H]2C[C@@H]42)C1 ; CN1CC(n2cc(C(F)(F)F)nc2-c2ccc3c(c2)OCCOc2ncnc(C4CC4)c2-c2ncc4c(n2)N(C3)C[C@H]2C[C@@H]42)C1]

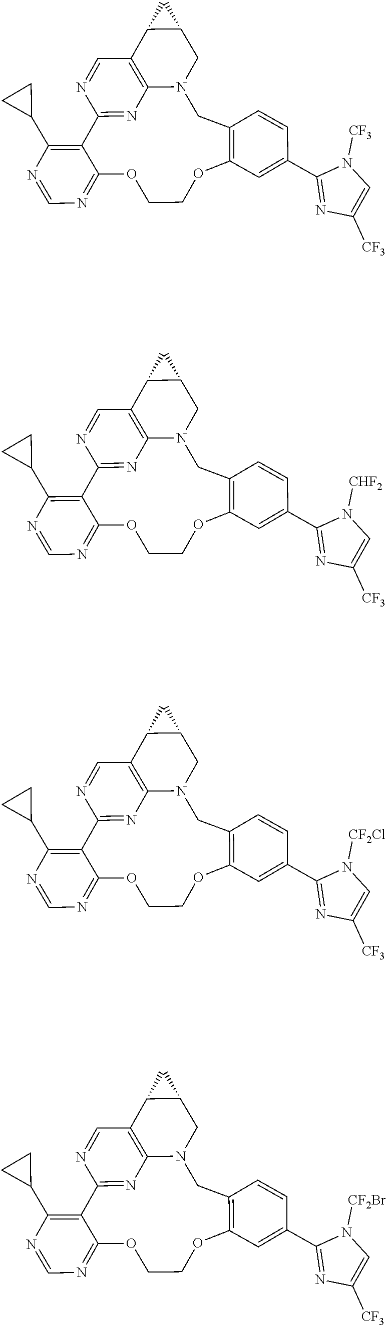

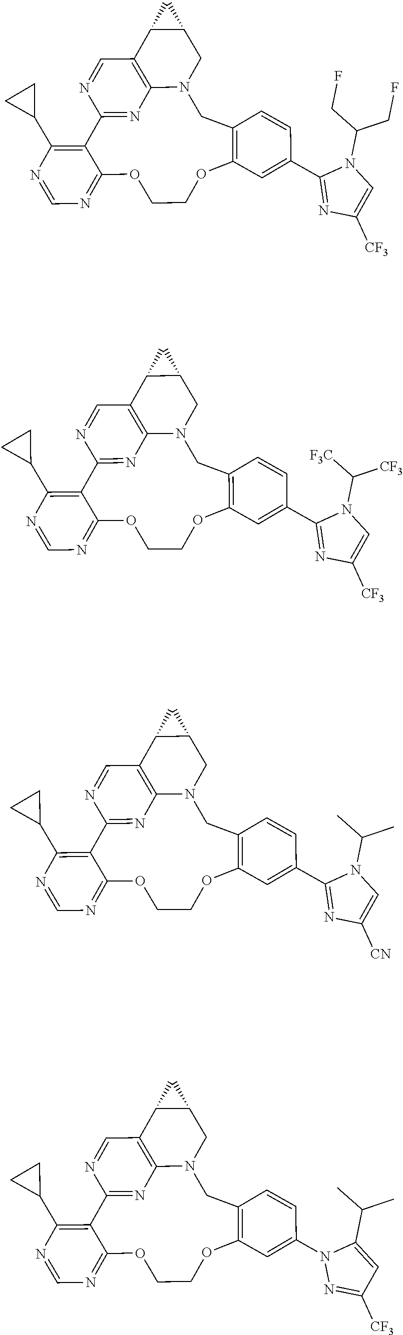 [SMILES: FCC(CF)n1cc(C(F)(F)F)nc1-c1ccc2c(c1)OCCOc1ncnc(C3CC3)c1-c1ncc3c(n1)N(C2)C[C@H]1C[C@@H]31 ; FC(F)(F)c1cn(C(C(F)(F)F)C(F)(F)F)c(-c2ccc3c(c2)OCCOc2ncnc(C4CC4)c2-c2ncc4c(n2)N(C3)C[C@H]2C[C@@H]42)n1 ; CC(C)n1cc(C#N)nc1-c1ccc2c(c1)OCCOc1ncnc(C3CC3)c1-c1ncc3c(n1)N(C2)C[C@H]1C[C@@H]31 ; CC(C)c1cc(C(F)(F)F)nn1-c1ccc2c(c1)OCCOc1ncnc(C3CC3)c1-c1ncc3c(n1)N(C2)C[C@H]1C[C@@H]31]

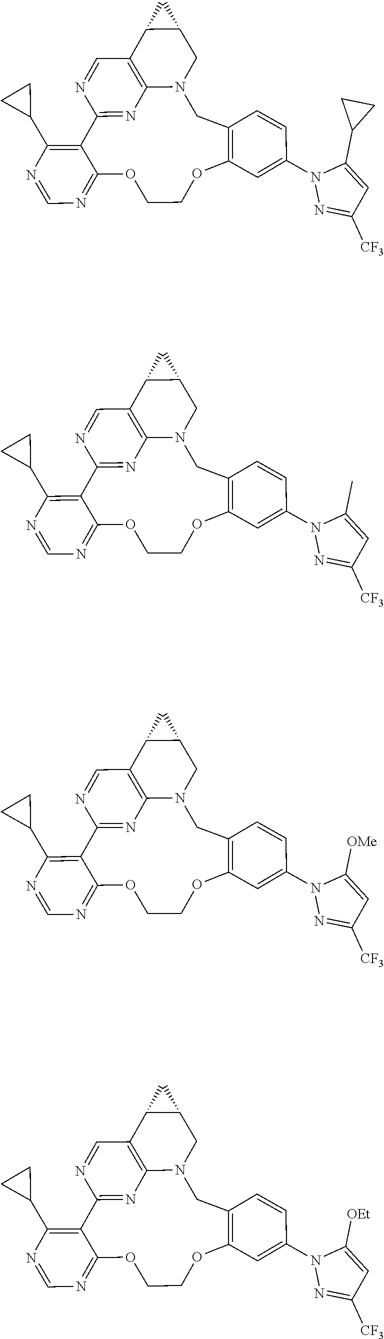 [SMILES: FC(F)(F)c1cc(C2CC2)n(-c2ccc3c(c2)OCCOc2ncnc(C4CC4)c2-c2ncc4c(n2)N(C3)C[C@H]2C[C@@H]42)n1 ; Cc1cc(C(F)(F)F)nn1-c1ccc2c(c1)OCCOc1ncnc(C3CC3)c1-c1ncc3c(n1)N(C2)C[C@H]1C[C@@H]31 ; COc1cc(C(F)(F)F)nn1-c1ccc2c(c1)OCCOc1ncnc(C3CC3)c1-c1ncc3c(n1)N(C2)C[C@H]1C[C@@H]31 ; CCOc1cc(C(F)(F)F)nn1-c1ccc2c(c1)OCCOc1ncnc(C3CC3)c1-c1ncc3c(n1)N(C2)C[C@H]1C[C@@H]31]

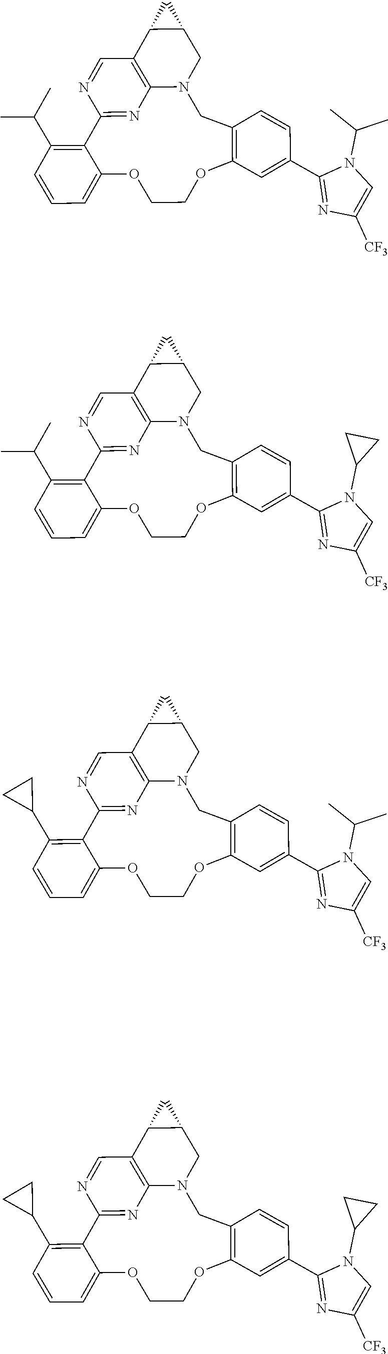

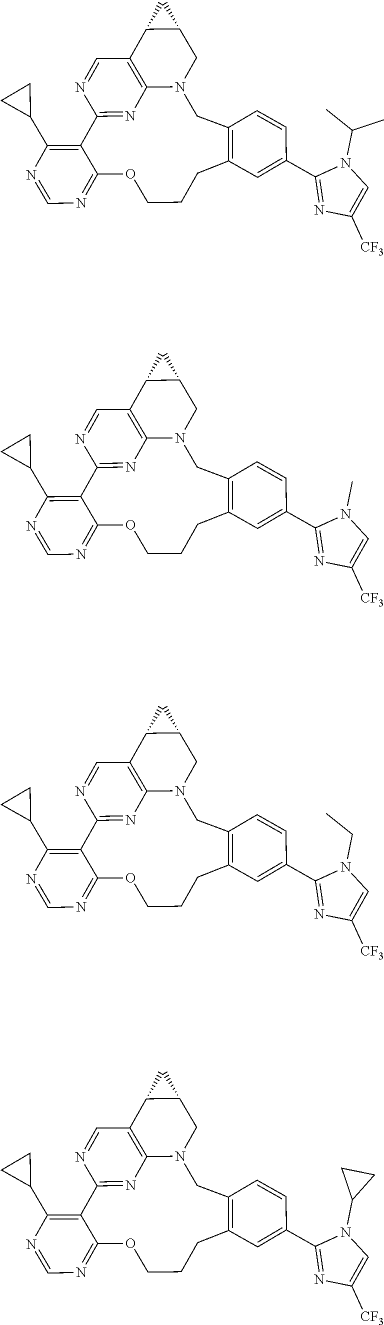 [SMILES: CC(C)n1cc(C(F)(F)F)nc1-c1ccc2c(c1)CCCOc1ncnc(C3CC3)c1-c1ncc3c(n1)N(C2)C[C@H]1C[C@@H]31 ; Cn1cc(C(F)(F)F)nc1-c1ccc2c(c1)CCCOc1ncnc(C3CC3)c1-c1ncc3c(n1)N(C2)C[C@H]1C[C@@H]31 ; CCn1cc(C(F)(F)F)nc1-c1ccc2c(c1)CCCOc1ncnc(C3CC3)c1-c1ncc3c(n1)N(C2)C[C@H]1C[C@@H]31 ; FC(F)(F)c1cn(C2CC2)c(-c2ccc3c(c2)CCCOc2ncnc(C4CC4)c2-c2ncc4c(n2)N(C3)C[C@H]2C[C@@H]42)n1]

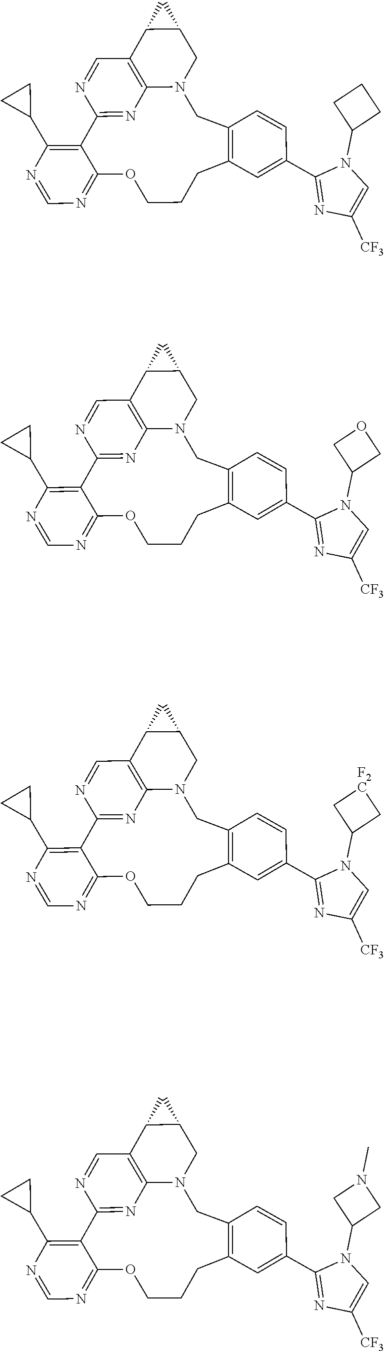 [SMILES: FC(F)(F)c1cn(C2CCC2)c(-c2ccc3c(c2)CCCOc2ncnc(C4CC4)c2-c2ncc4c(n2)N(C3)C[C@H]2C[C@@H]42)n1 ; FC(F)(F)c1cn(C2COC2)c(-c2ccc3c(c2)CCCOc2ncnc(C4CC4)c2-c2ncc4c(n2)N(C3)C[C@H]2C[C@@H]42)n1 ; FC1(F)CC(n2cc(C(F)(F)F)nc2-c2ccc3c(c2)CCCOc2ncnc(C4CC4)c2-c2ncc4c(n2)N(C3)C[C@H]2C[C@@H]42)C1 ; CN1CC(n2cc(C(F)(F)F)nc2-c2ccc3c(c2)CCCOc2ncnc(C4CC4)c2-c2ncc4c(n2)N(C3)C[C@H]2C[C@@H]42)C1]

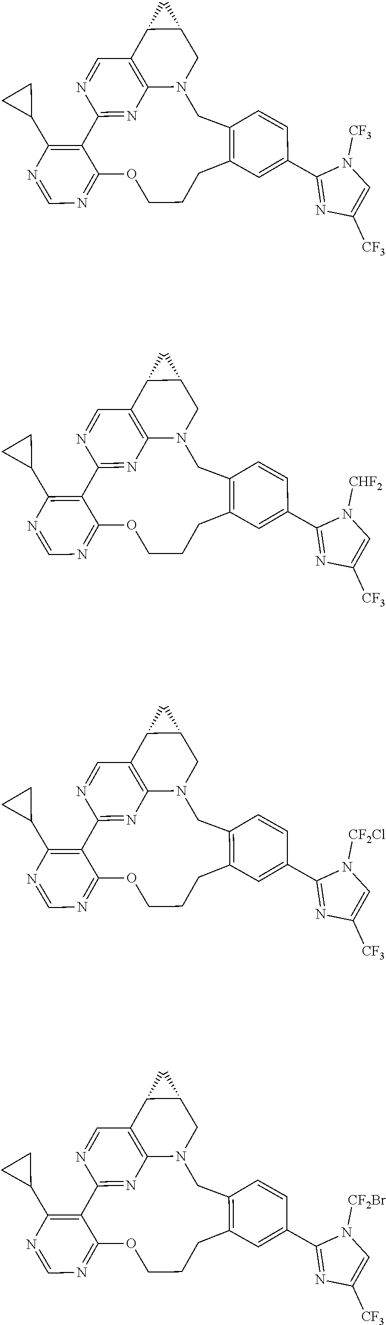

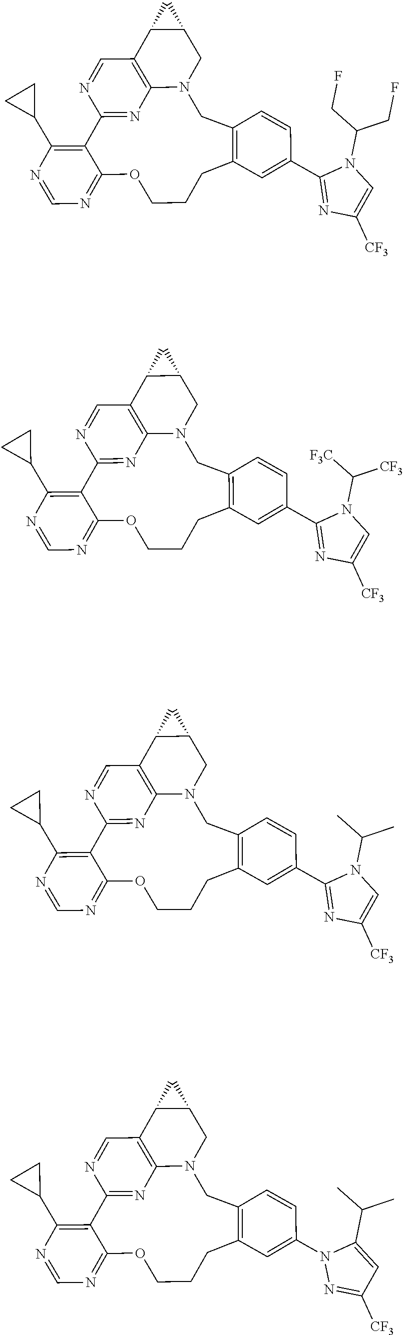 [SMILES: FCC(CF)n1cc(C(F)(F)F)nc1-c1ccc2c(c1)CCCOc1ncnc(C3CC3)c1-c1ncc3c(n1)N(C2)C[C@H]1C[C@@H]31 ; FC(F)(F)c1cn(C(C(F)(F)F)C(F)(F)F)c(-c2ccc3c(c2)CCCOc2ncnc(C4CC4)c2-c2ncc4c(n2)N(C3)C[C@H]2C[C@@H]42)n1 ; CC(C)n1cc(C(F)(F)F)nc1-c1ccc2c(c1)CCCOc1ncnc(C3CC3)c1-c1ncc3c(n1)N(C2)C[C@H]1C[C@@H]31 ; CC(C)c1cc(C(F)(F)F)nn1-c1ccc2c(c1)CCCOc1ncnc(C3CC3)c1-c1ncc3c(n1)N(C2)C[C@H]1C[C@@H]31]

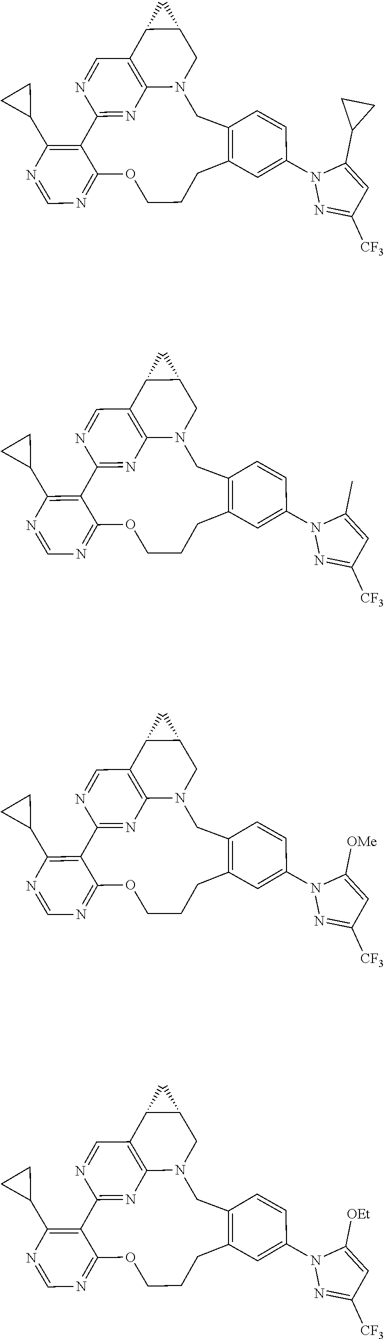 [SMILES: FC(F)(F)c1cc(C2CC2)n(-c2ccc3c(c2)CCCOc2ncnc(C4CC4)c2-c2ncc4c(n2)N(C3)C[C@H]2C[C@@H]42)n1 ; Cc1cc(C(F)(F)F)nn1-c1ccc2c(c1)CCCOc1ncnc(C3CC3)c1-c1ncc3c(n1)N(C2)C[C@H]1C[C@@H]31 ; COc1cc(C(F)(F)F)nn1-c1ccc2c(c1)CCCOc1ncnc(C3CC3)c1-c1ncc3c(n1)N(C2)C[C@H]1C[C@@H]31 ; CCOc1cc(C(F)(F)F)nn1-c1ccc2c(c1)CCCOc1ncnc(C3CC3)c1-c1ncc3c(n1)N(C2)C[C@H]1C[C@@H]31]

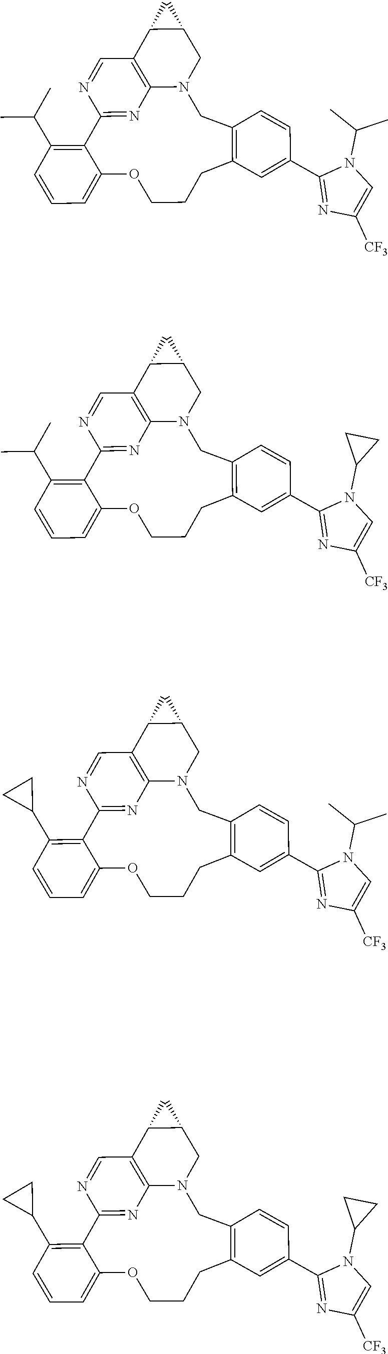

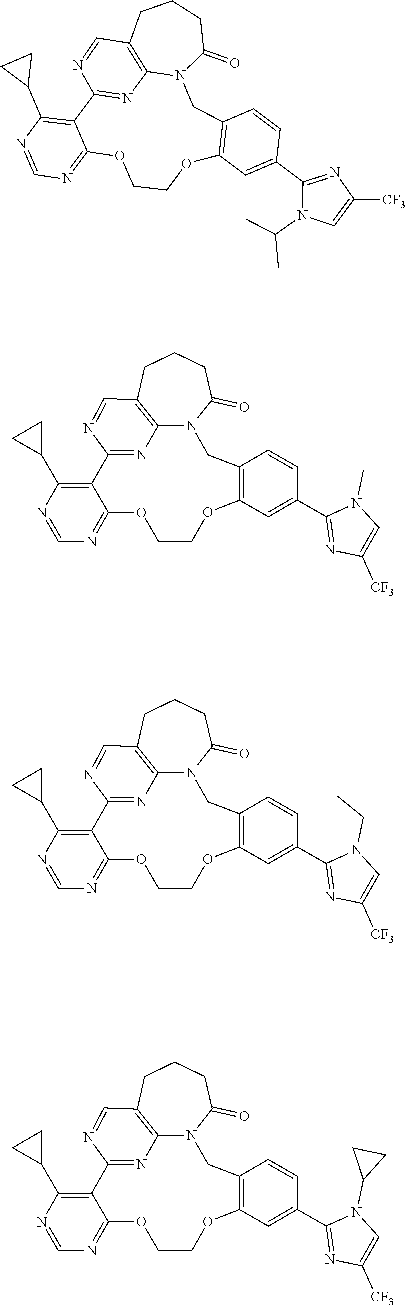 [SMILES: CC(C)n1cc(C(F)(F)F)nc1-c1ccc2c(c1)OCCOc1ncnc(C3CC3)c1-c1ncc3c(n1)N(C2)C(=O)CCC3 ; Cn1cc(C(F)(F)F)nc1-c1ccc2c(c1)OCCOc1ncnc(C3CC3)c1-c1ncc3c(n1)N(C2)C(=O)CCC3 ; CCn1cc(C(F)(F)F)nc1-c1ccc2c(c1)OCCOc1ncnc(C3CC3)c1-c1ncc3c(n1)N(C2)C(=O)CCC3 ; O=C1CCCc2cnc3nc2N1Cc1ccc(-c2nc(C(F)(F)F)cn2C2CC2)cc1OCCOc1ncnc(C2CC2)c1-3]

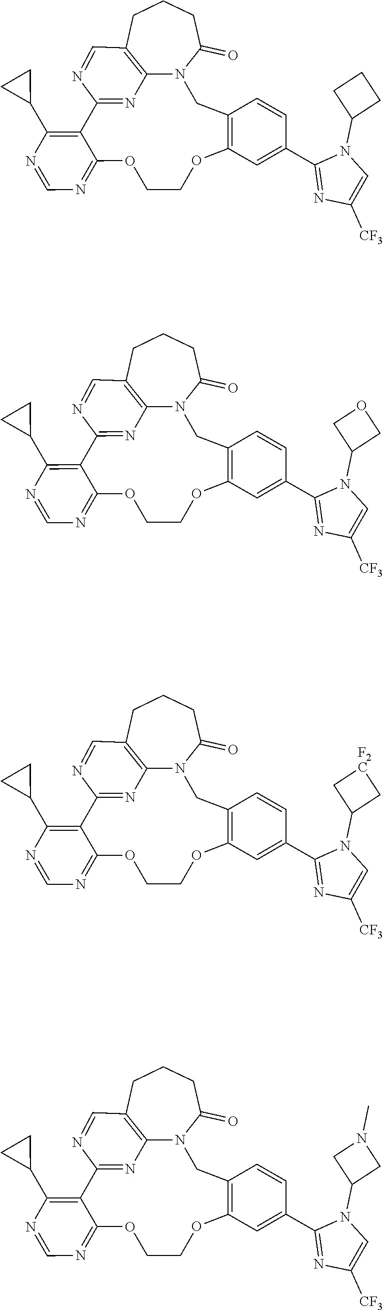 [SMILES: O=C1CCCc2cnc3nc2N1Cc1ccc(-c2nc(C(F)(F)F)cn2C2CCC2)cc1OCCOc1ncnc(C2CC2)c1-3 ; O=C1CCCc2cnc3nc2N1Cc1ccc(-c2nc(C(F)(F)F)cn2C2COC2)cc1OCCOc1ncnc(C2CC2)c1-3 ; O=C1CCCc2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)CCC4)C1]

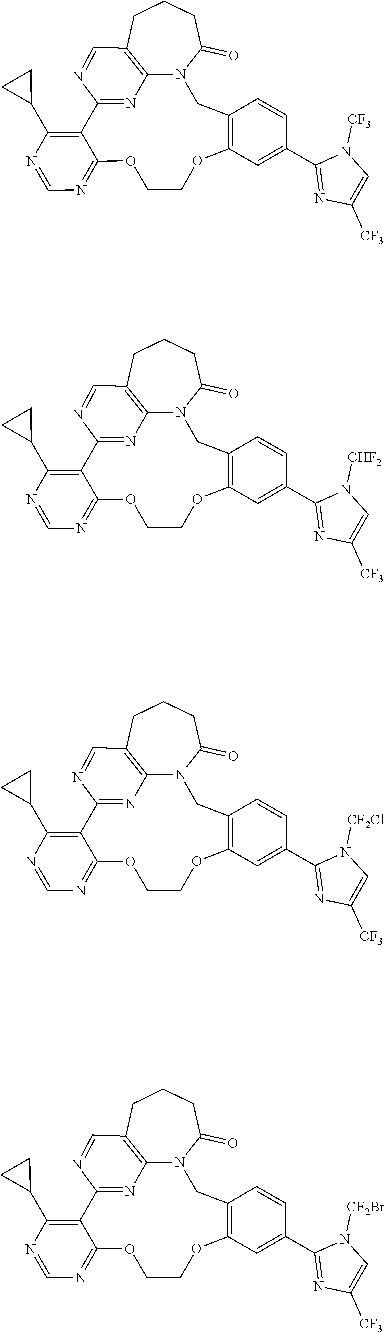

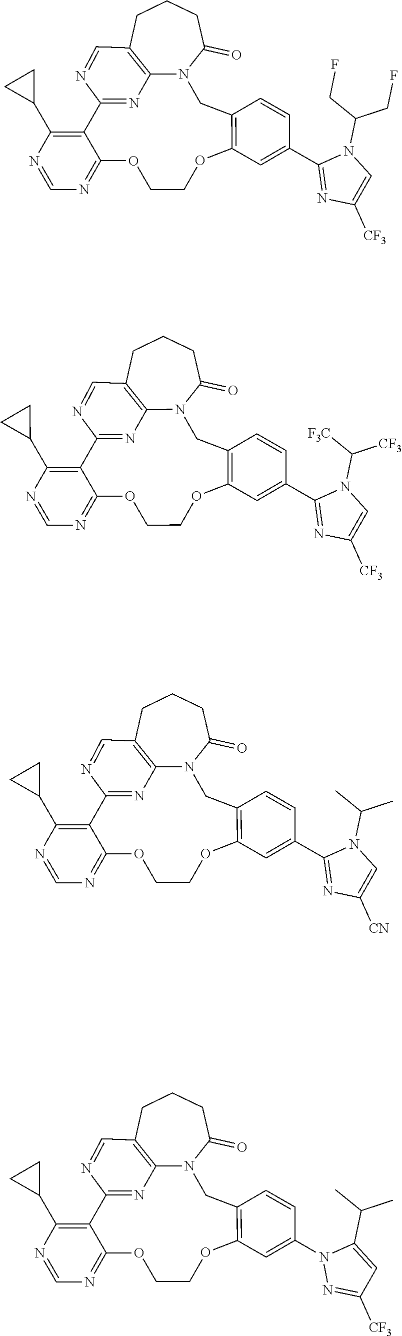 [SMILES: O=C1CCCc2cnc3nc2N1Cc1ccc(-c2nc(C(F)(F)F)cn2C(CF)CF)cc1OCCOc1ncnc(C2CC2)c1-3 ; O=C1CCCc2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)CCC3 ; CC(C)c1cc(C(F)(F)F)nn1-c1ccc2c(c1)OCCOc1ncnc(C3CC3)c1-c1ncc3c(n1)N(C2)C(=O)CCC3]

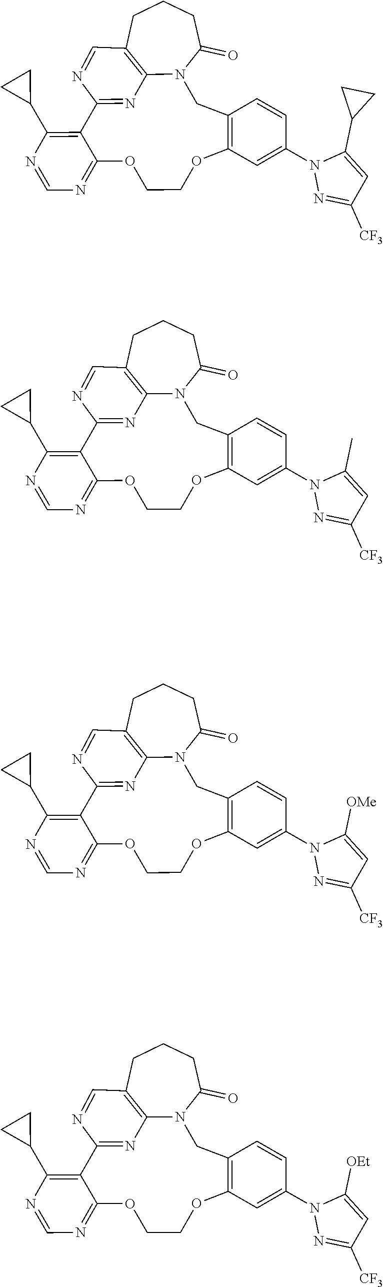 [SMILES: O=C1CCCc2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)CCC3 ; COc1cc(C(F)(F)F)nn1-c1ccc2c(c1)OCCOc1ncnc(C3CC3)c1-c1ncc3c(n1)N(C2)C(=O)CCC3 ; CCOc1cc(C(F)(F)F)nn1-c1ccc2c(c1)OCCOc1ncnc(C3CC3)c1-c1ncc3c(n1)N(C2)C(=O)CCC3]

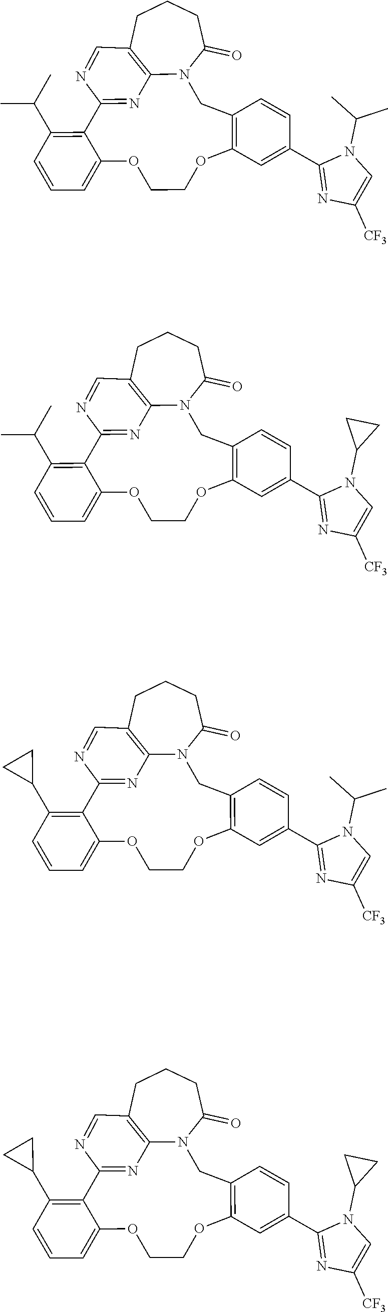

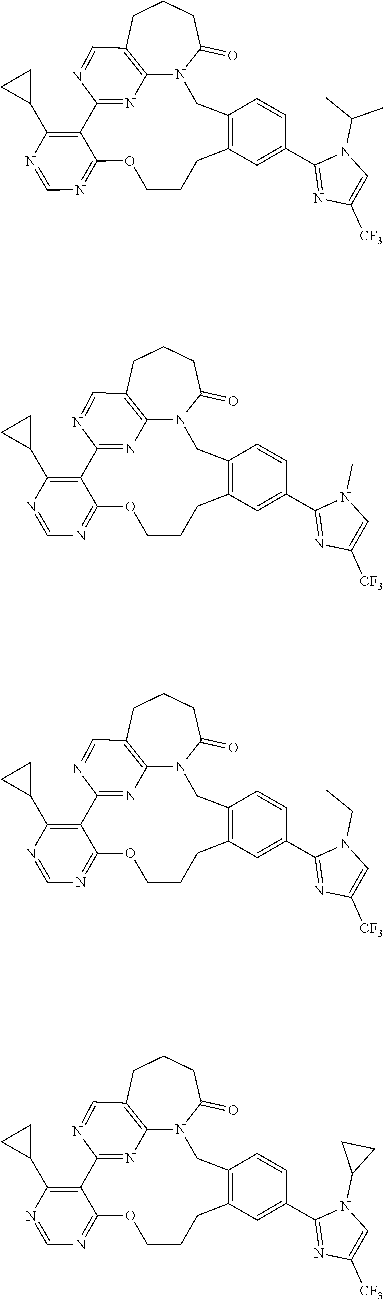 [SMILES: CC(C)n1cc(C(F)(F)F)nc1-c1ccc2c(c1)CCCOc1ncnc(C3CC3)c1-c1ncc3c(n1)N(C2)C(=O)CCC3 ; Cn1cc(C(F)(F)F)nc1-c1ccc2c(c1)CCCOc1ncnc(C3CC3)c1-c1ncc3c(n1)N(C2)C(=O)CCC3 ; CCn1cc(C(F)(F)F)nc1-c1ccc2c(c1)CCCOc1ncnc(C3CC3)c1-c1ncc3c(n1)N(C2)C(=O)CCC3 ; O=C1CCCc2cnc3nc2N1Cc1ccc(-c2nc(C(F)(F)F)cn2C2CC2)cc1CCCOc1ncnc(C2CC2)c1-3]

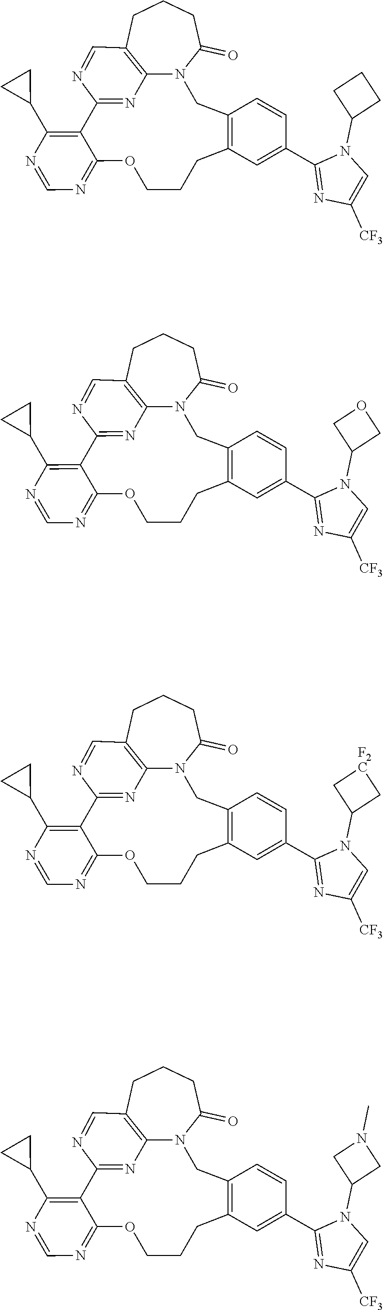 [SMILES: O=C1CCCc2cnc3nc2N1Cc1ccc(-c2nc(C(F)(F)F)cn2C2CCC2)cc1CCCOc1ncnc(C2CC2)c1-3 ; O=C1CCCc2cnc3nc2N1Cc1ccc(-c2nc(C(F)(F)F)cn2C2COC2)cc1CCCOc1ncnc(C2CC2)c1-3 ; O=C1CCCc2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)CCC4)C1]

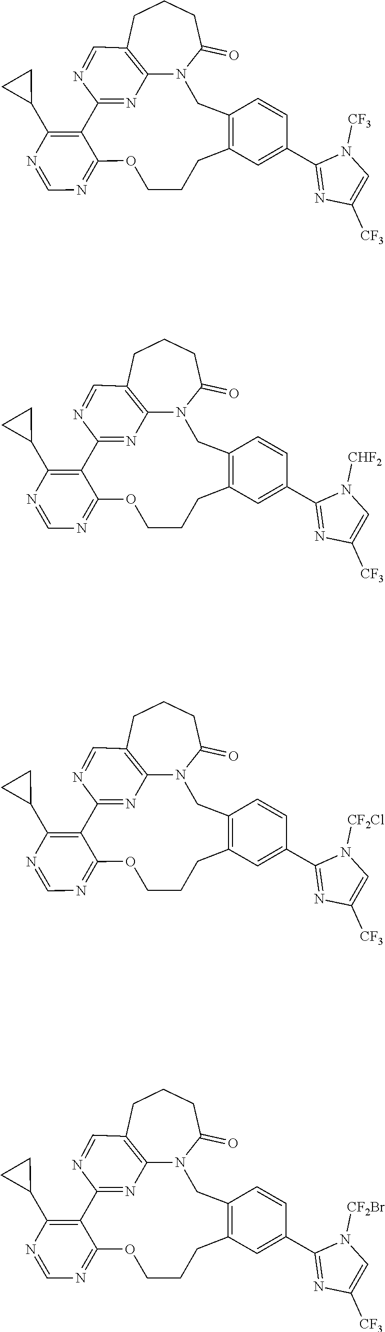

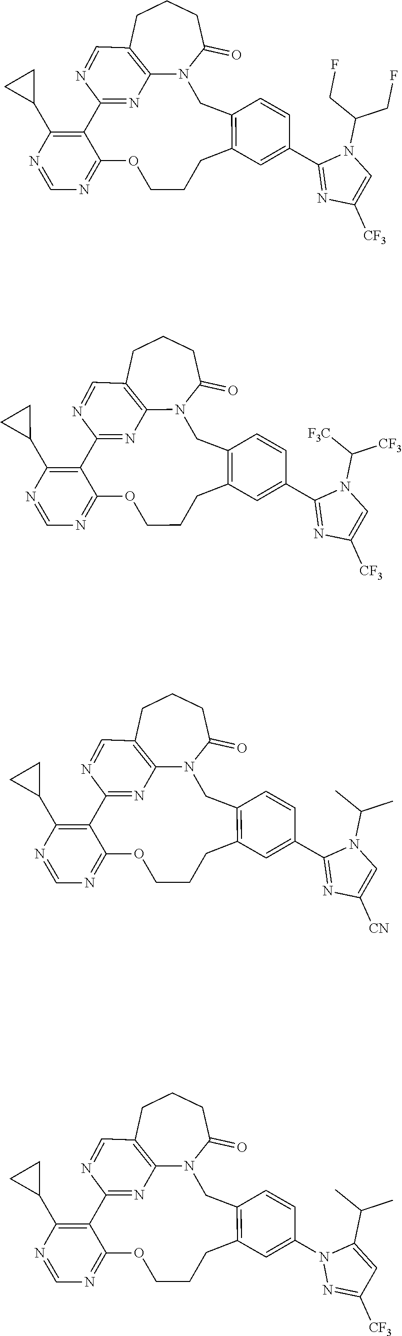 [SMILES: O=C1CCCc2cnc3nc2N1Cc1ccc(-c2nc(C(F)(F)F)cn2C(CF)CF)cc1CCCOc1ncnc(C2CC2)c1-3 ; O=C1CCCc2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)CCC3 ; CC(C)c1cc(C(F)(F)F)nn1-c1ccc2c(c1)CCCOc1ncnc(C3CC3)c1-c1ncc3c(n1)N(C2)C(=O)CCC3]

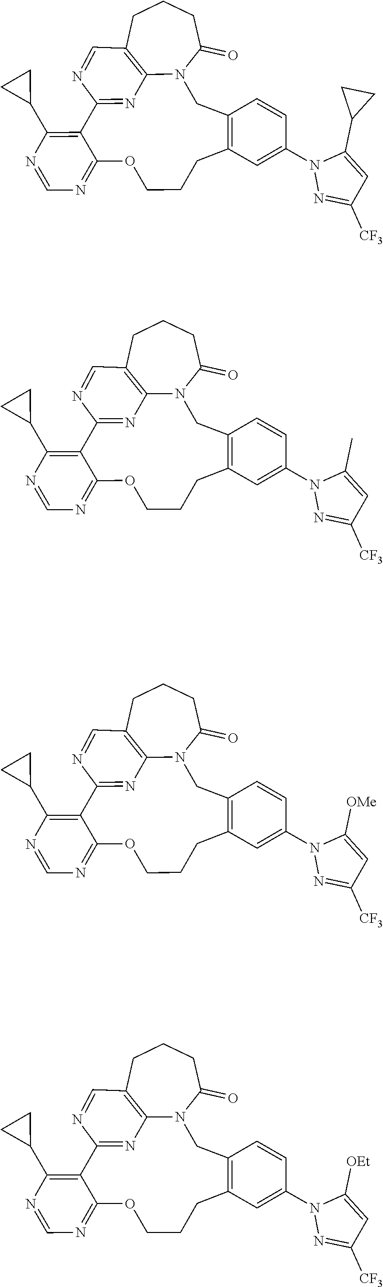 [SMILES: O=C1CCCc2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)CCC3 ; COc1cc(C(F)(F)F)nn1-c1ccc2c(c1)CCCOc1ncnc(C3CC3)c1-c1ncc3c(n1)N(C2)C(=O)CCC3 ; CCOc1cc(C(F)(F)F)nn1-c1ccc2c(c1)CCCOc1ncnc(C3CC3)c1-c1ncc3c(n1)N(C2)C(=O)CCC3]

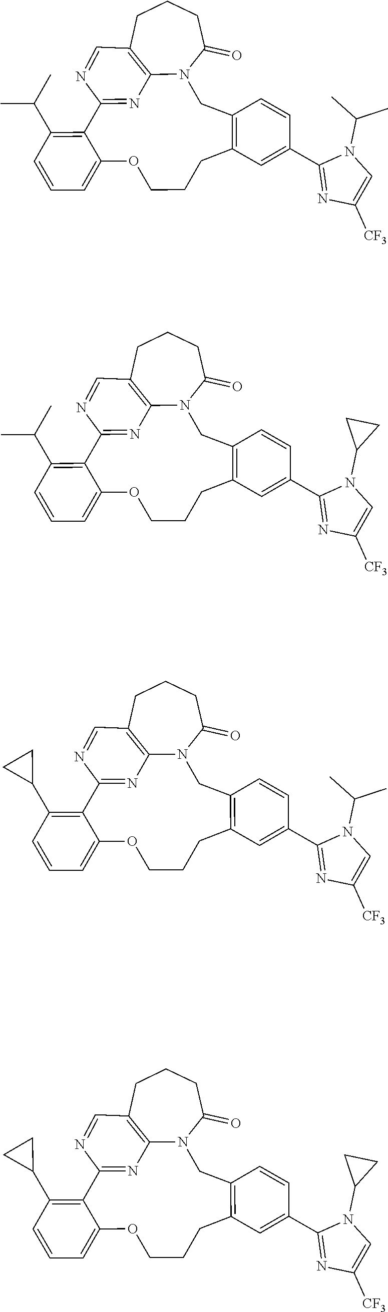

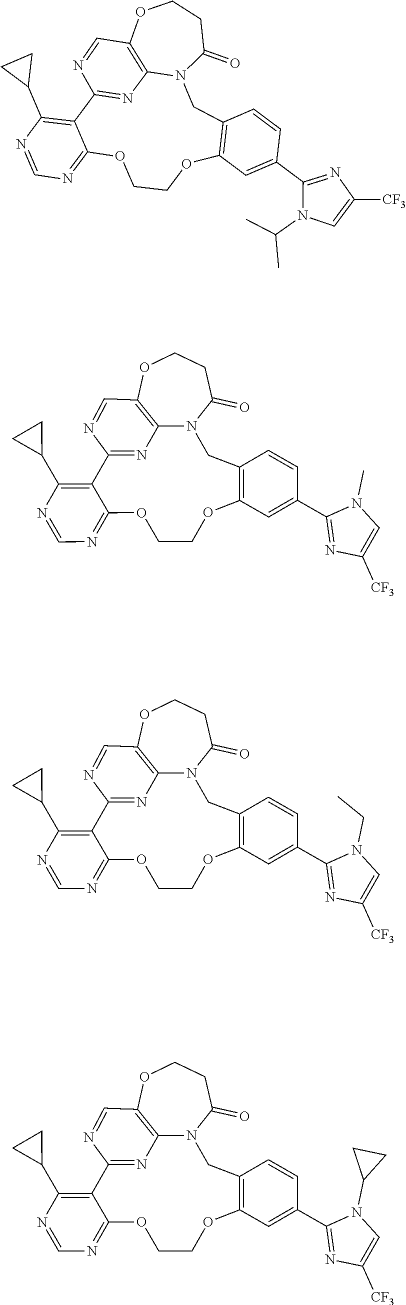 [SMILES: CC(C)n1cc(C(F)(F)F)nc1-c1ccc2c(c1)OCCOc1ncnc(C3CC3)c1-c1ncc3c(n1)N(C2)C(=O)CCO3 ; Cn1cc(C(F)(F)F)nc1-c1ccc2c(c1)OCCOc1ncnc(C3CC3)c1-c1ncc3c(n1)N(C2)C(=O)CCO3 ; CCn1cc(C(F)(F)F)nc1-c1ccc2c(c1)OCCOc1ncnc(C3CC3)c1-c1ncc3c(n1)N(C2)C(=O)CCO3 ; O=C1CCOc2cnc3nc2N1Cc1ccc(-c2nc(C(F)(F)F)cn2C2CC2)cc1OCCOc1ncnc(C2CC2)c1-3]

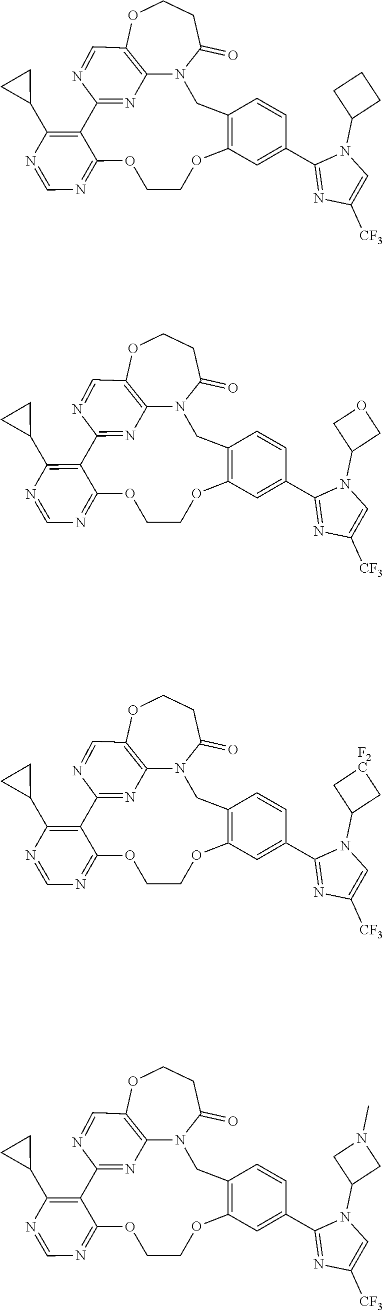 [SMILES: O=C1CCOc2cnc3nc2N1Cc1ccc(-c2nc(C(F)(F)F)cn2C2CCC2)cc1OCCOc1ncnc(C2CC2)c1-3 ; O=C1CCOc2cnc3nc2N1Cc1ccc(-c2nc(C(F)(F)F)cn2C2COC2)cc1OCCOc1ncnc(C2CC2)c1-3 ; O=C1CCOc2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)CCO4)C1]

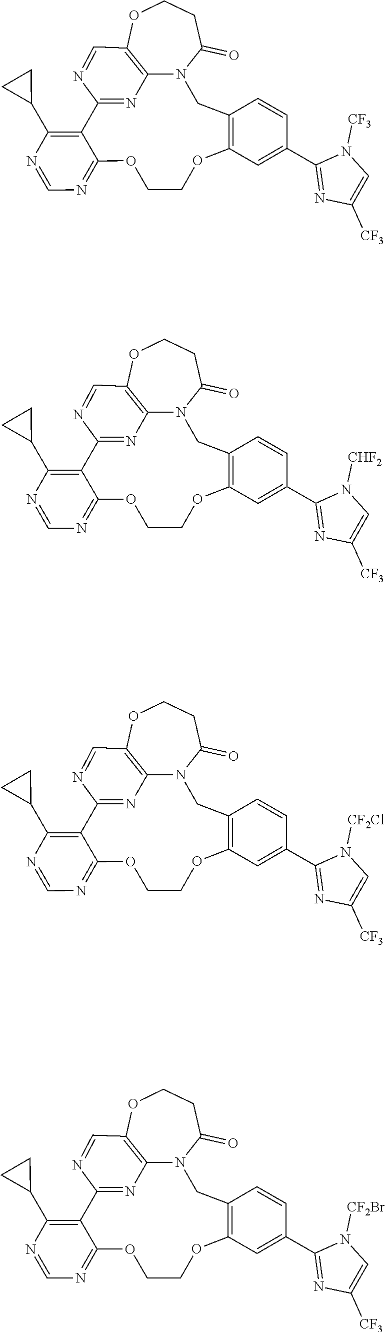

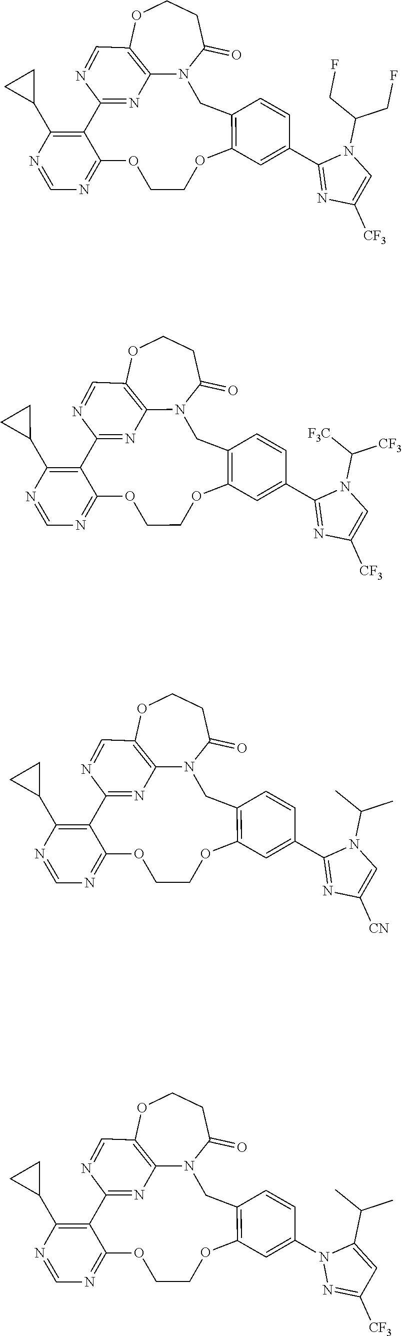 [SMILES: O=C1CCOc2cnc3nc2N1Cc1ccc(-c2nc(C(F)(F)F)cn2C(CF)CF)cc1OCCOc1ncnc(C2CC2)c1-3 ; O=C1CCOc2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)CCO3 ; CC(C)c1cc(C(F)(F)F)nn1-c1ccc2c(c1)OCCOc1ncnc(C3CC3)c1-c1ncc3c(n1)N(C2)C(=O)CCO3]

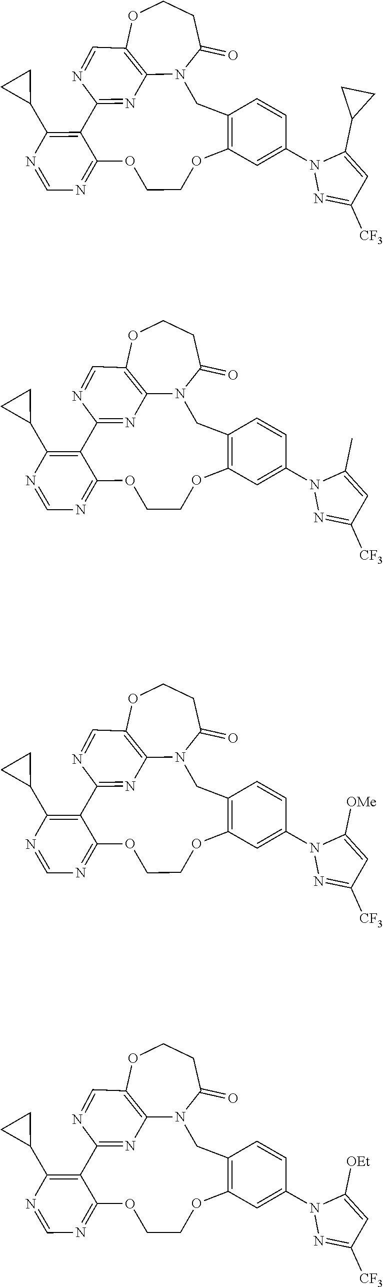 [SMILES: O=C1CCOc2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)CCO3 ; COc1cc(C(F)(F)F)nn1-c1ccc2c(c1)OCCOc1ncnc(C3CC3)c1-c1ncc3c(n1)N(C2)C(=O)CCO3 ; CCOc1cc(C(F)(F)F)nn1-c1ccc2c(c1)OCCOc1ncnc(C3CC3)c1-c1ncc3c(n1)N(C2)C(=O)CCO3]

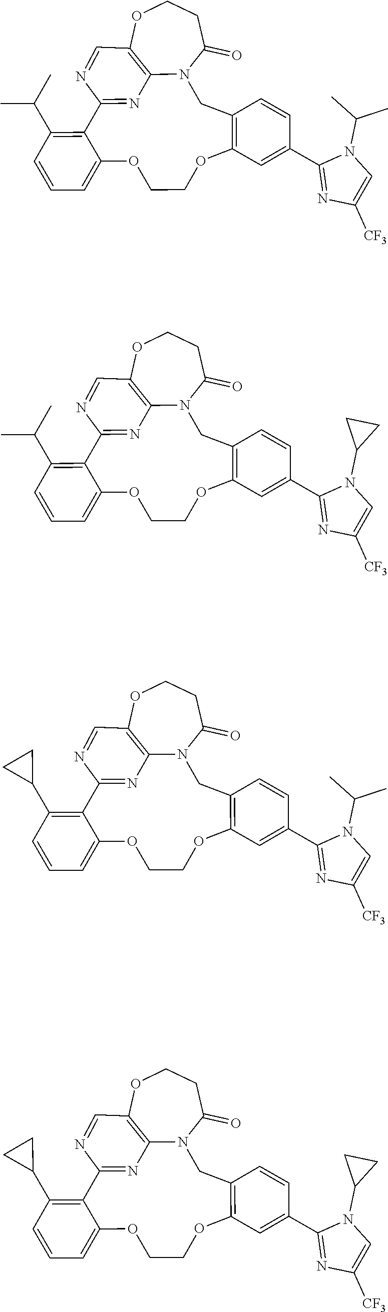

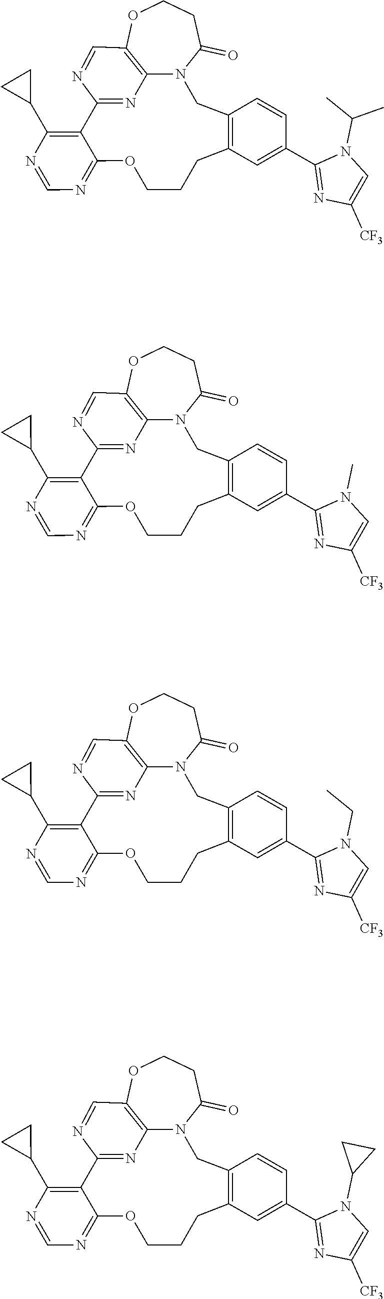 [SMILES: CC(C)n1cc(C(F)(F)F)nc1-c1ccc2c(c1)CCCOc1ncnc(C3CC3)c1-c1ncc3c(n1)N(C2)C(=O)CCO3 ; Cn1cc(C(F)(F)F)nc1-c1ccc2c(c1)CCCOc1ncnc(C3CC3)c1-c1ncc3c(n1)N(C2)C(=O)CCO3 ; CCn1cc(C(F)(F)F)nc1-c1ccc2c(c1)CCCOc1ncnc(C3CC3)c1-c1ncc3c(n1)N(C2)C(=O)CCO3 ; O=C1CCOc2cnc3nc2N1Cc1ccc(-c2nc(C(F)(F)F)cn2C2CC2)cc1CCCOc1ncnc(C2CC2)c1-3]

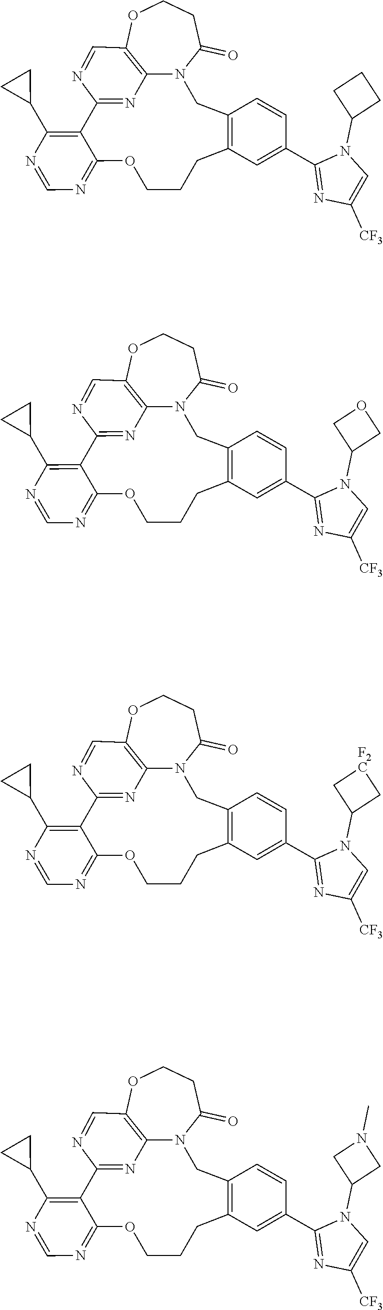 [SMILES: O=C1CCOc2cnc3nc2N1Cc1ccc(-c2nc(C(F)(F)F)cn2C2CCC2)cc1CCCOc1ncnc(C2CC2)c1-3 ; O=C1CCOc2cnc3nc2N1Cc1ccc(-c2nc(C(F)(F)F)cn2C2COC2)cc1CCCOc1ncnc(C2CC2)c1-3 ; O=C1CCOc2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)CCO4)C1]

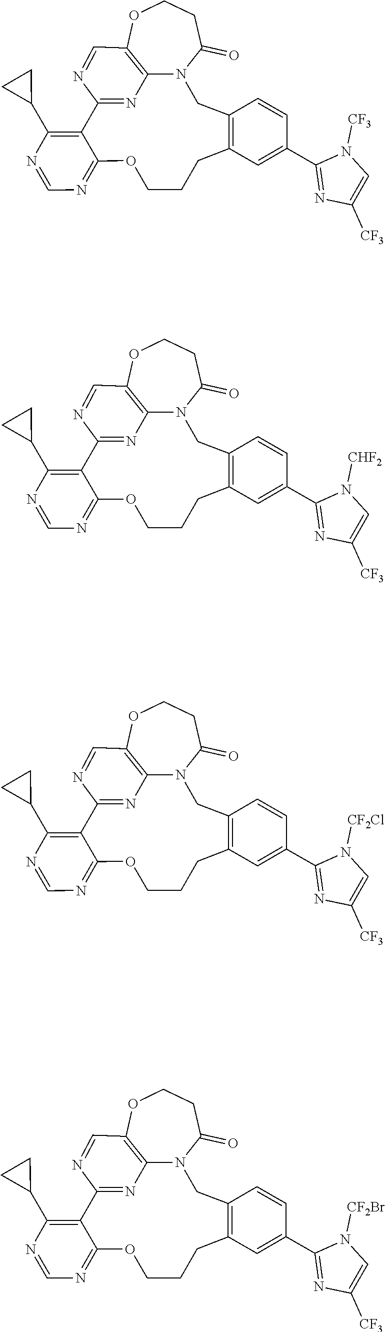

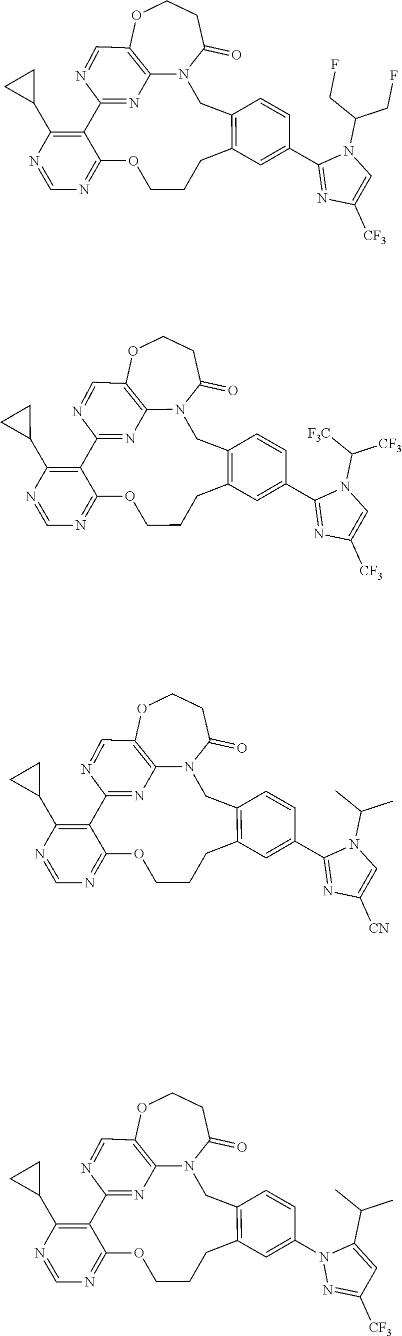 [SMILES: O=C1CCOc2cnc3nc2N1Cc1ccc(-c2nc(C(F)(F)F)cn2C(CF)CF)cc1CCCOc1ncnc(C2CC2)c1-3 ; O=C1CCOc2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)CCO3 ; CC(C)c1cc(C(F)(F)F)nn1-c1ccc2c(c1)CCCOc1ncnc(C3CC3)c1-c1ncc3c(n1)N(C2)C(=O)CCO3]

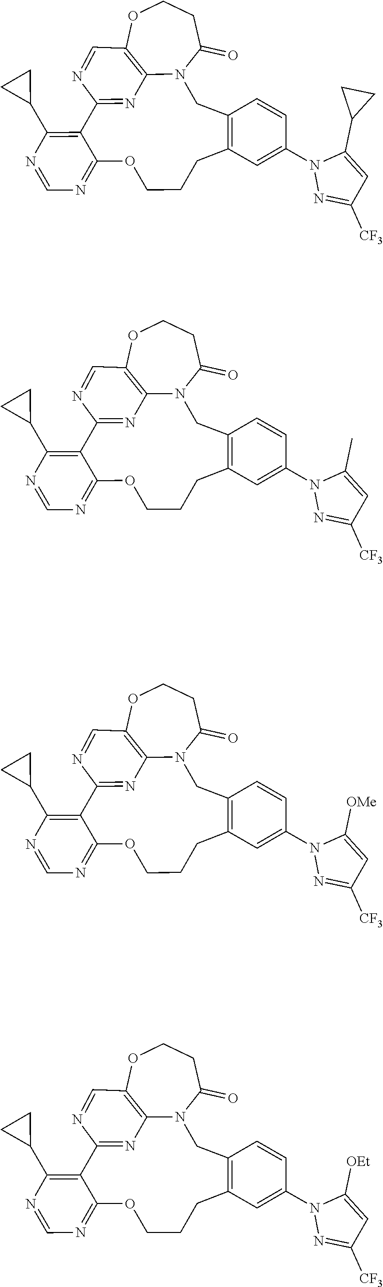 [SMILES: O=C1CCOc2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)CCO3 ; COc1cc(C(F)(F)F)nn1-c1ccc2c(c1)CCCOc1ncnc(C3CC3)c1-c1ncc3c(n1)N(C2)C(=O)CCO3 ; CCOc1cc(C(F)(F)F)nn1-c1ccc2c(c1)CCCOc1ncnc(C3CC3)c1-c1ncc3c(n1)N(C2)C(=O)CCO3]

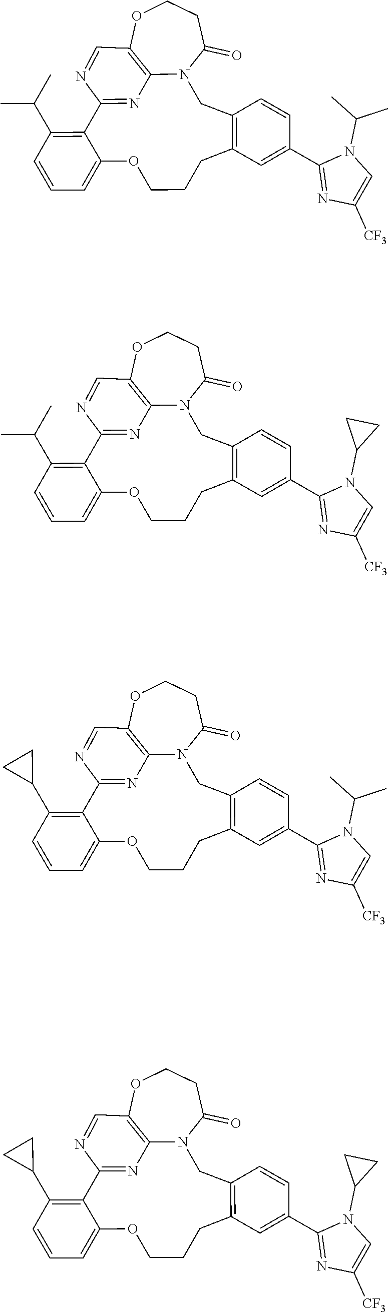

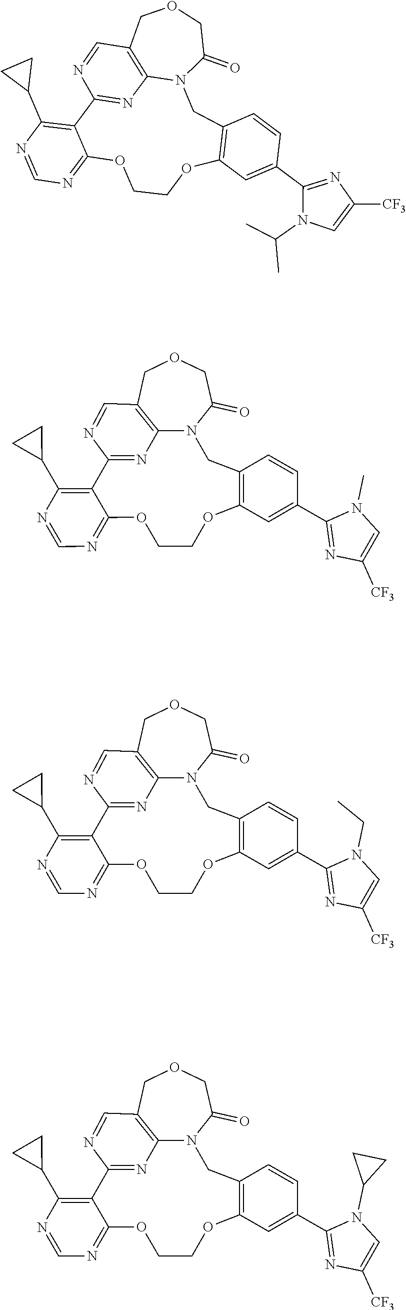 [SMILES: CC(C)n1cc(C(F)(F)F)nc1-c1ccc2c(c1)OCCOc1ncnc(C3CC3)c1-c1ncc3c(n1)N(C2)C(=O)COC3 ; Cn1cc(C(F)(F)F)nc1-c1ccc2c(c1)OCCOc1ncnc(C3CC3)c1-c1ncc3c(n1)N(C2)C(=O)COC3 ; CCn1cc(C(F)(F)F)nc1-c1ccc2c(c1)OCCOc1ncnc(C3CC3)c1-c1ncc3c(n1)N(C2)C(=O)COC3 ; O=C1COCc2cnc3nc2N1Cc1ccc(-c2nc(C(F)(F)F)cn2C2CC2)cc1OCCOc1ncnc(C2CC2)c1-3]

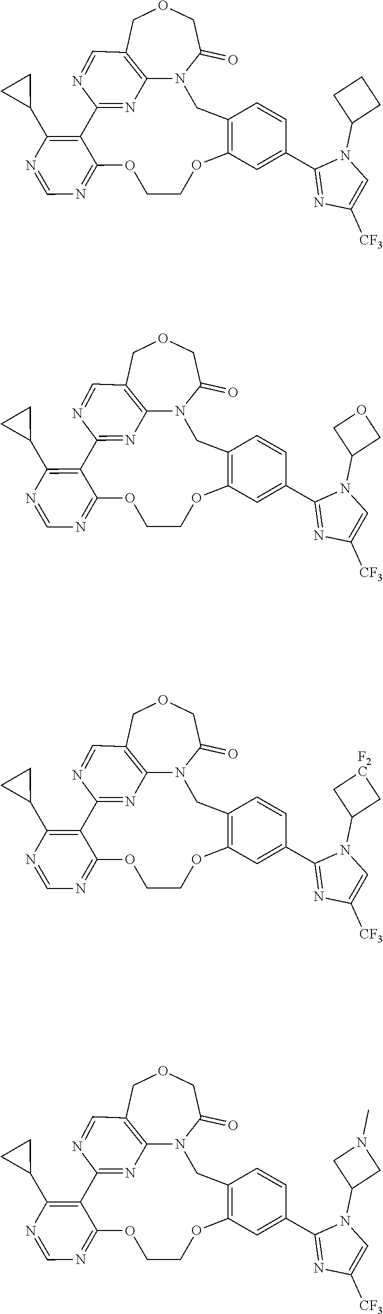 [SMILES: O=C1COCc2cnc3nc2N1Cc1ccc(-c2nc(C(F)(F)F)cn2C2CCC2)cc1OCCOc1ncnc(C2CC2)c1-3 ; O=C1COCc2cnc3nc2N1Cc1ccc(-c2nc(C(F)(F)F)cn2C2COC2)cc1OCCOc1ncnc(C2CC2)c1-3 ; O=C1COCc2cnc3nc2N1Cc1ccc(-c2nc(C(F)(F)F)cn2C2CC(F)(F)C2)cc1OCCOc1ncnc(C2CC2)c1-3 ; CN1CC(n2cc(C(F)(F)F)nc2-c2ccc3c(c2)OCCOc2ncnc(C4CC4)c2-c2ncc4c(n2)N(C3)C(=O)COC4)C1]

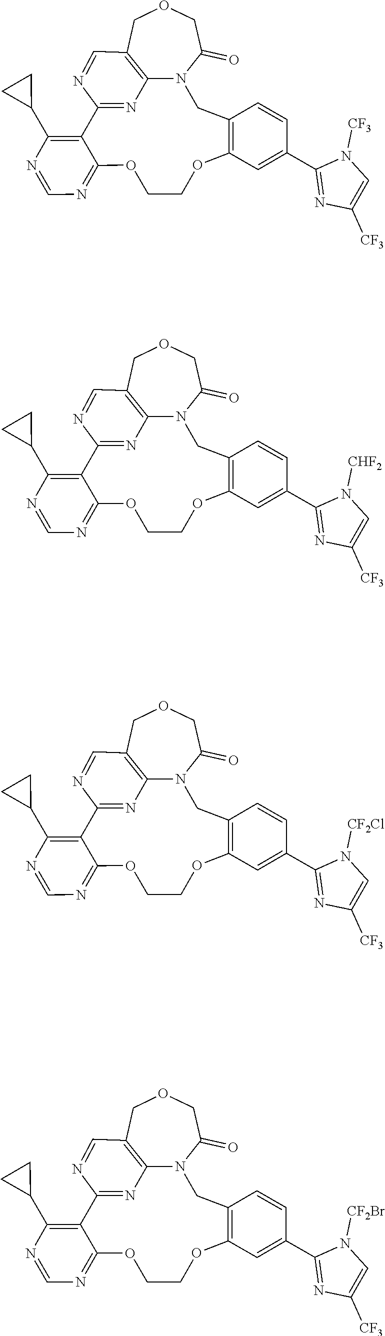

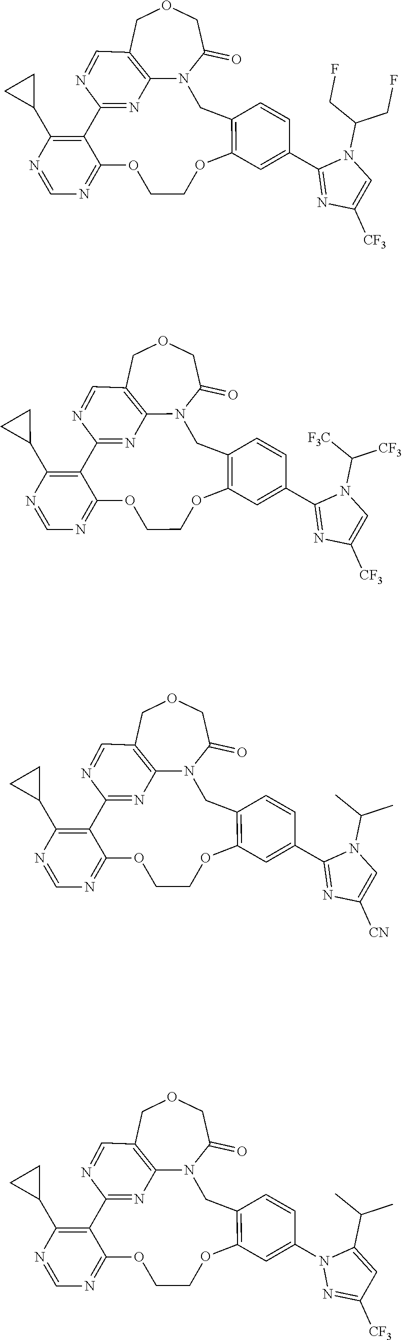 [SMILES: O=C1COCc2cnc3nc2N1Cc1ccc(-c2nc(C(F)(F)F)cn2C(CF)CF)cc1OCCOc1ncnc(C2CC2)c1-3 ; O=C1COCc2cnc3nc2N1Cc1ccc(-c2nc(C(F)(F)F)cn2C(C(F)(F)F)C(F)(F)F)cc1OCCOc1ncnc(C2CC2)c1-3 ; CC(C)n1cc(C#N)nc1-c1ccc2c(c1)OCCOc1ncnc(C3CC3)c1-c1ncc3c(n1)N(C2)C(=O)COC3 ; CC(C)c1cc(C(F)(F)F)nn1-c1ccc2c(c1)OCCOc1ncnc(C3CC3)c1-c1ncc3c(n1)N(C2)C(=O)COC3]

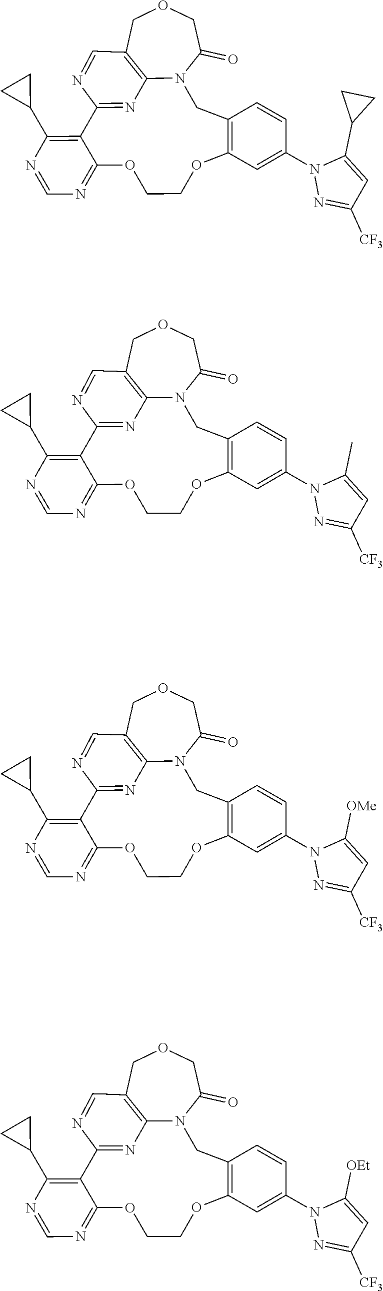 [SMILES: O=C1COCc2cnc3nc2N1Cc1ccc(-n2nc(C(F)(F)F)cc2C2CC2)cc1OCCOc1ncnc(C2CC2)c1-3 ; Cc1cc(C(F)(F)F)nn1-c1ccc2c(c1)OCCOc1ncnc(C3CC3)c1-c1ncc3c(n1)N(C2)C(=O)COC3 ; COc1cc(C(F)(F)F)nn1-c1ccc2c(c1)OCCOc1ncnc(C3CC3)c1-c1ncc3c(n1)N(C2)C(=O)COC3 ; CCOc1cc(C(F)(F)F)nn1-c1ccc2c(c1)OCCOc1ncnc(C3CC3)c1-c1ncc3c(n1)N(C2)C(=O)COC3]

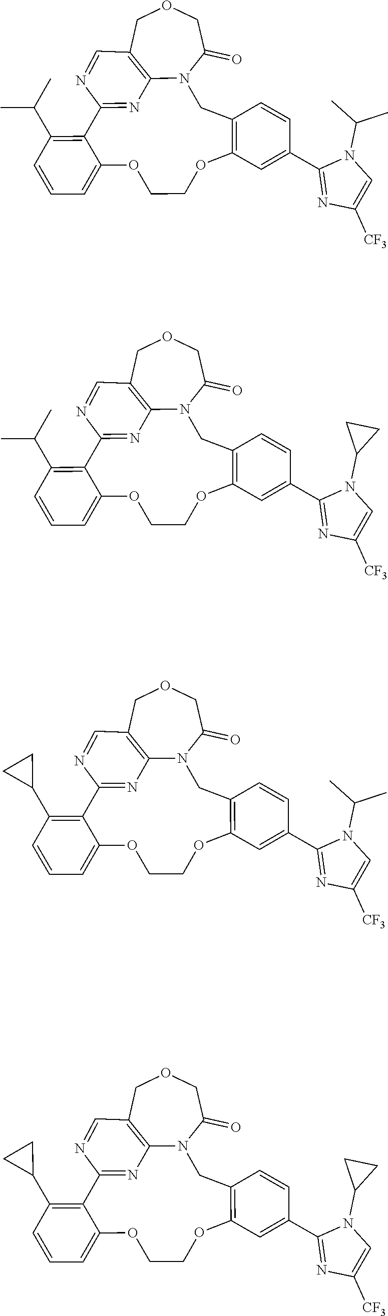

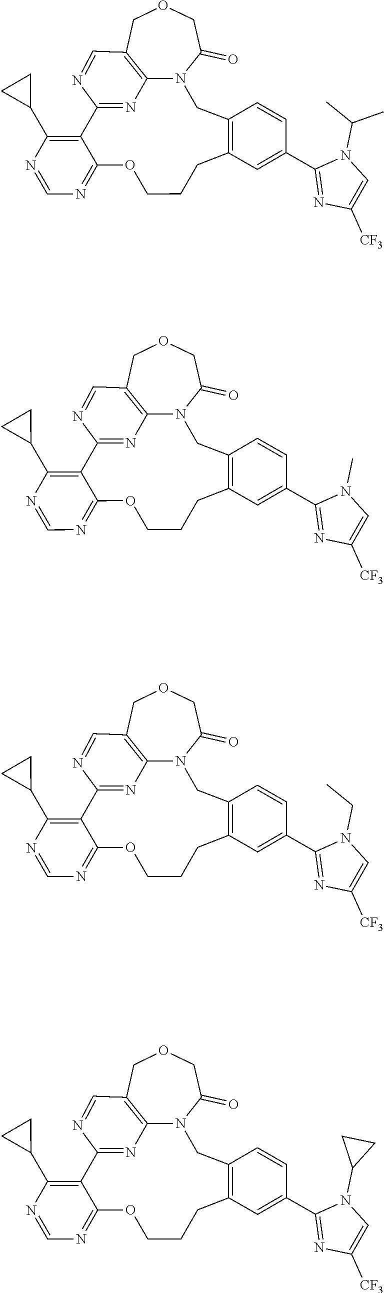 [SMILES: CC(C)n1cc(C(F)(F)F)nc1-c1ccc2c(c1)CCCOc1ncnc(C3CC3)c1-c1ncc3c(n1)N(C2)C(=O)COC3 ; Cn1cc(C(F)(F)F)nc1-c1ccc2c(c1)CCCOc1ncnc(C3CC3)c1-c1ncc3c(n1)N(C2)C(=O)COC3 ; CCn1cc(C(F)(F)F)nc1-c1ccc2c(c1)CCCOc1ncnc(C3CC3)c1-c1ncc3c(n1)N(C2)C(=O)COC3 ; O=C1COCc2cnc3nc2N1Cc1ccc(-c2nc(C(F)(F)F)cn2C2CC2)cc1CCCOc1ncnc(C2CC2)c1-3]

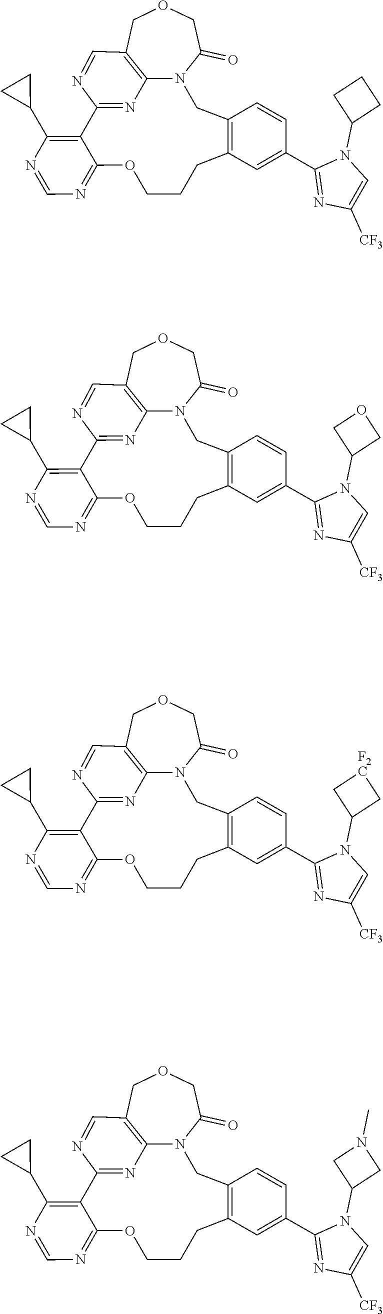 [SMILES: O=C1COCc2cnc3nc2N1Cc1ccc(-c2nc(C(F)(F)F)cn2C2CCC2)cc1CCCOc1ncnc(C2CC2)c1-3 ; O=C1COCc2cnc3nc2N1Cc1ccc(-c2nc(C(F)(F)F)cn2C2COC2)cc1CCCOc1ncnc(C2CC2)c1-3 ; O=C1COCc2cnc3nc2N1Cc1ccc(-c2nc(C(F)(F)F)cn2C2CC(F)(F)C2)cc1CCCOc1ncnc(C2CC2)c1-3 ; CN1CC(n2cc(C(F)(F)F)nc2-c2ccc3c(c2)CCCOc2ncnc(C4CC4)c2-c2ncc4c(n2)N(C3)C(=O)COC4)C1]

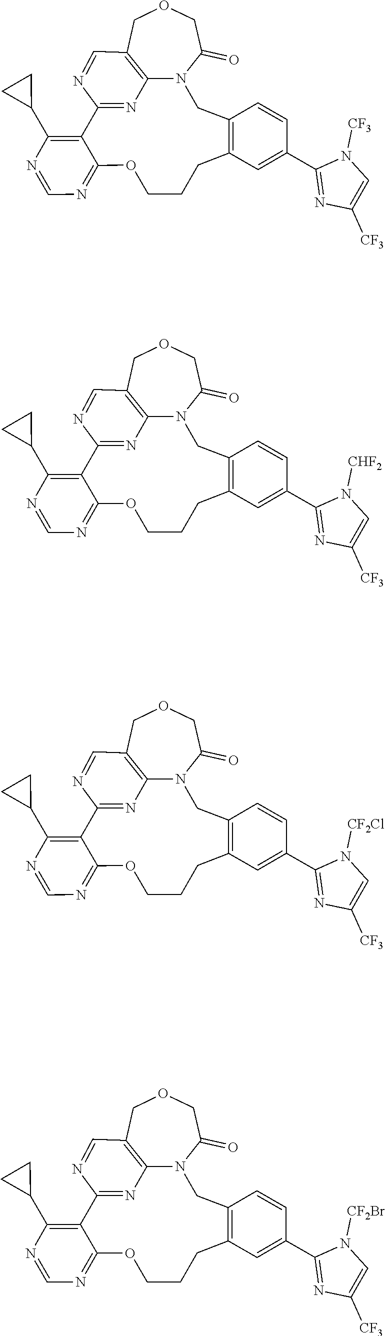

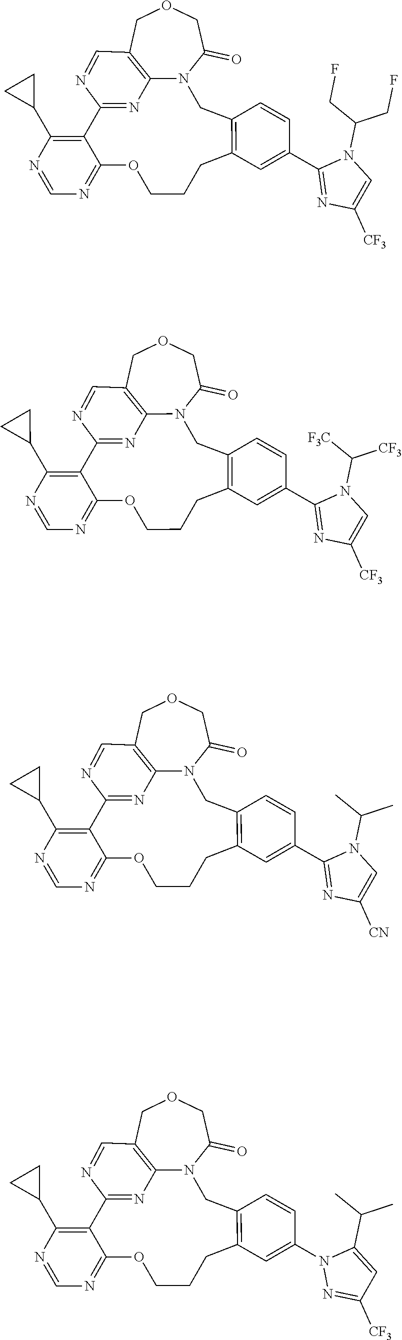 [SMILES: O=C1COCc2cnc3nc2N1Cc1ccc(-c2nc(C(F)(F)F)cn2C(CF)CF)cc1CCCOc1ncnc(C2CC2)c1-3 ; O=C1COCc2cnc3nc2N1Cc1ccc(-c2nc(C(F)(F)F)cn2C(C(F)(F)F)C(F)(F)F)cc1CCCOc1ncnc(C2CC2)c1-3 ; CC(C)n1cc(C#N)nc1-c1ccc2c(c1)CCCOc1ncnc(C3CC3)c1-c1ncc3c(n1)N(C2)C(=O)COC3 ; CC(C)c1cc(C(F)(F)F)nn1-c1ccc2c(c1)CCCOc1ncnc(C3CC3)c1-c1ncc3c(n1)N(C2)C(=O)COC3]

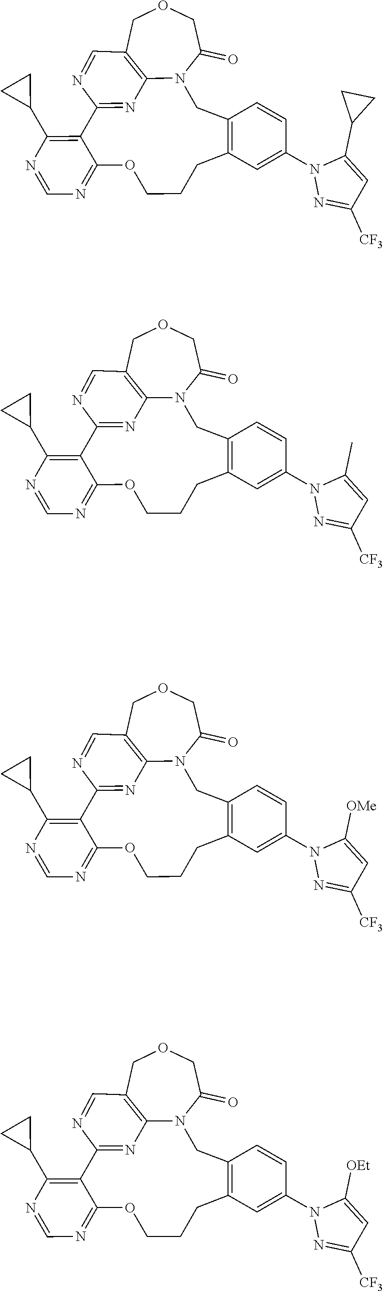 [SMILES: O=C1COCc2cnc3nc2N1Cc1ccc(-n2nc(C(F)(F)F)cc2C2CC2)cc1CCCOc1ncnc(C2CC2)c1-3 ; Cc1cc(C(F)(F)F)nn1-c1ccc2c(c1)CCCOc1ncnc(C3CC3)c1-c1ncc3c(n1)N(C2)C(=O)COC3 ; COc1cc(C(F)(F)F)nn1-c1ccc2c(c1)CCCOc1ncnc(C3CC3)c1-c1ncc3c(n1)N(C2)C(=O)COC3 ; CCOc1cc(C(F)(F)F)nn1-c1ccc2c(c1)CCCOc1ncnc(C3CC3)c1-c1ncc3c(n1)N(C2)C(=O)COC3]

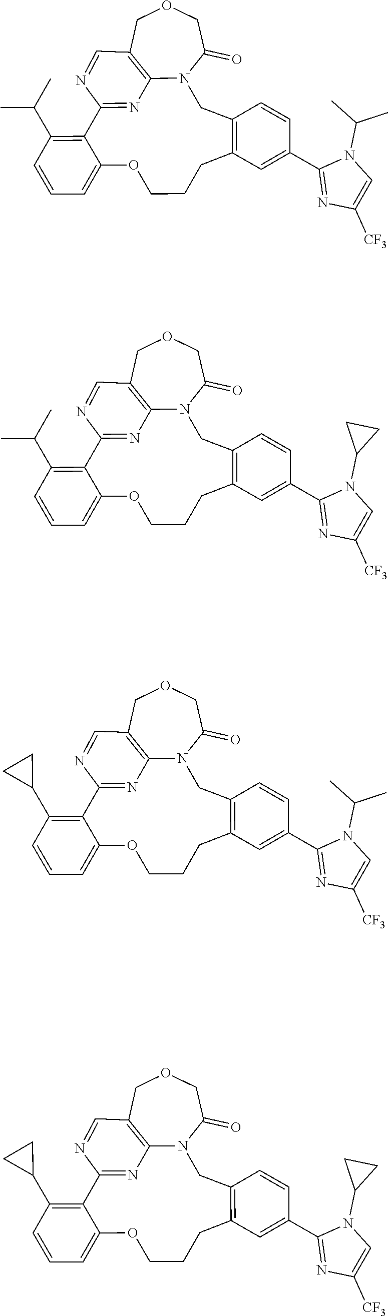

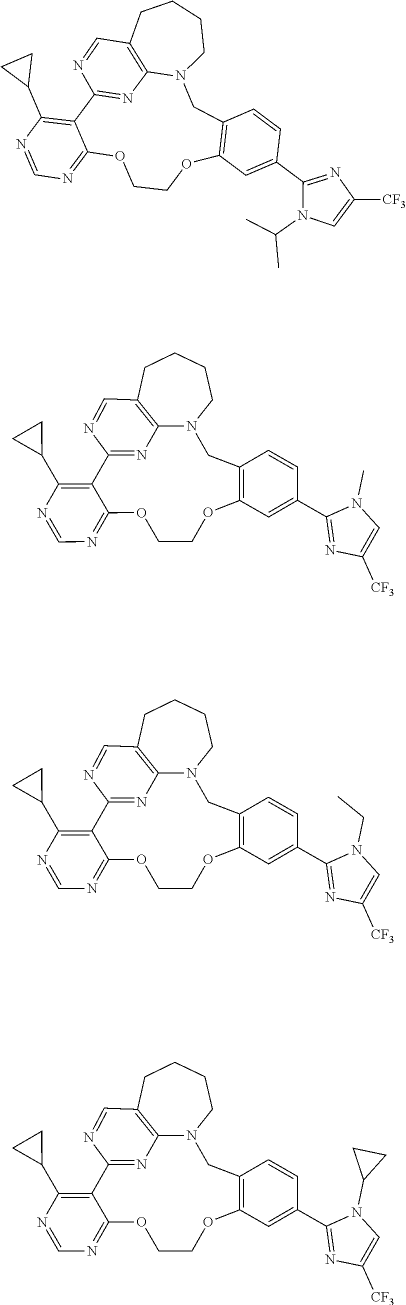 [SMILES: CC(C)n1cc(C(F)(F)F)nc1-c1ccc2c(c1)OCCOc1ncnc(C3CC3)c1-c1ncc3c(n1)N(CCCC3)C2 ; Cn1cc(C(F)(F)F)nc1-c1ccc2c(c1)OCCOc1ncnc(C3CC3)c1-c1ncc3c(n1)N(CCCC3)C2 ; CCn1cc(C(F)(F)F)nc1-c1ccc2c(c1)OCCOc1ncnc(C3CC3)c1-c1ncc3c(n1)N(CCCC3)C2 ; FC(F)(F)c1cn(C2CC2)c(-c2ccc3c(c2)OCCOc2ncnc(C4CC4)c2-c2ncc4c(n2)N(CCCC4)C3)n1]

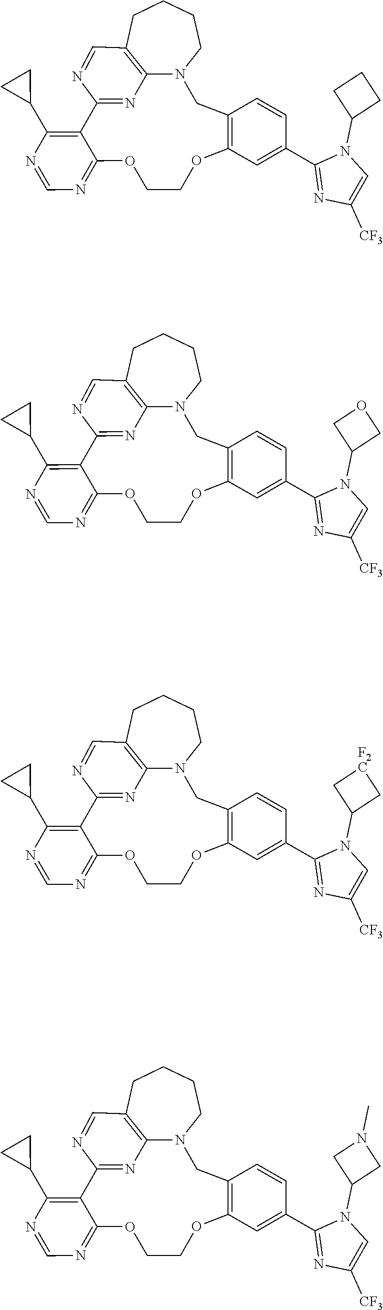 [SMILES: FC(F)(F)c1cn(C2CCC2)c(-c2ccc3c(c2)OCCOc2ncnc(C4CC4)c2-c2ncc4c(n2)N(CCCC4)C3)n1 ; FC(F)(F)c1cn(C2COC2)c(-c2ccc3c(c2)OCCOc2ncnc(C4CC4)c2-c2ncc4c(n2)N(CCCC4)C3)n1 ; FC1(F)CC(n2cc(C(F)(F)F)nc2-c2ccc3c(c2)OCCOc2ncnc(C4CC4)c2-c2ncc4c(n2)N(CCCC4)C3)C1 ; CN1CC(n2cc(C(F)(F)F)nc2-c2ccc3c(c2)OCCOc2ncnc(C4CC4)c2-c2ncc4c(n2)N(CCCC4)C3)C1]

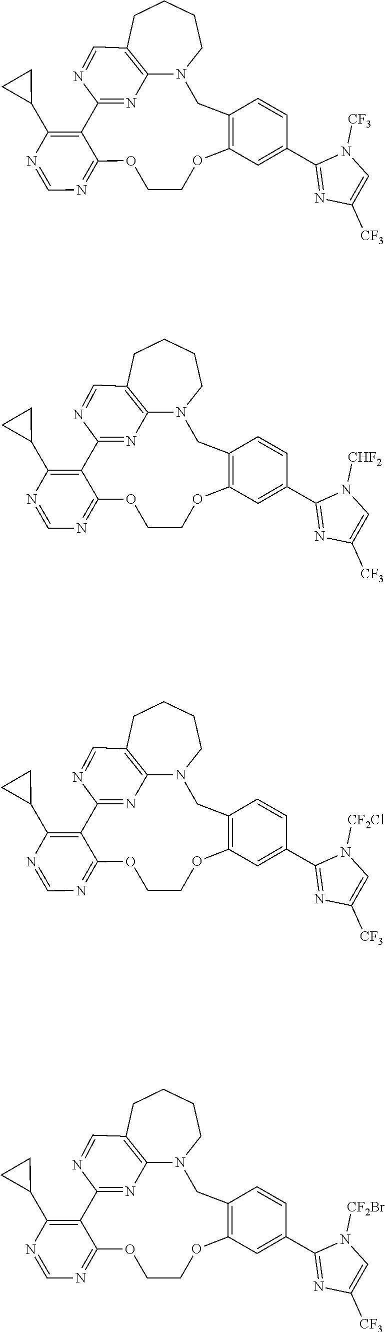

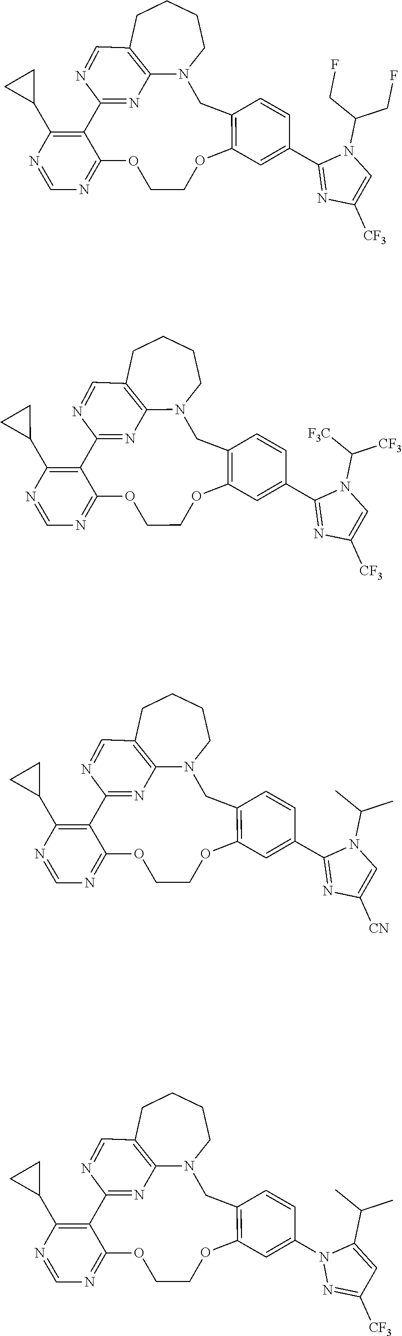 [SMILES: FCC(CF)n1cc(C(F)(F)F)nc1-c1ccc2c(c1)OCCOc1ncnc(C3CC3)c1-c1ncc3c(n1)N(CCCC3)C2 ; FC(F)(F)c1cn(C(C(F)(F)F)C(F)(F)F)c(-c2ccc3c(c2)OCCOc2ncnc(C4CC4)c2-c2ncc4c(n2)N(CCCC4)C3)n1 ; CC(C)n1cc(C#N)nc1-c1ccc2c(c1)OCCOc1ncnc(C3CC3)c1-c1ncc3c(n1)N(CCCC3)C2 ; CC(C)c1cc(C(F)(F)F)nn1-c1ccc2c(c1)OCCOc1ncnc(C3CC3)c1-c1ncc3c(n1)N(CCCC3)C2]

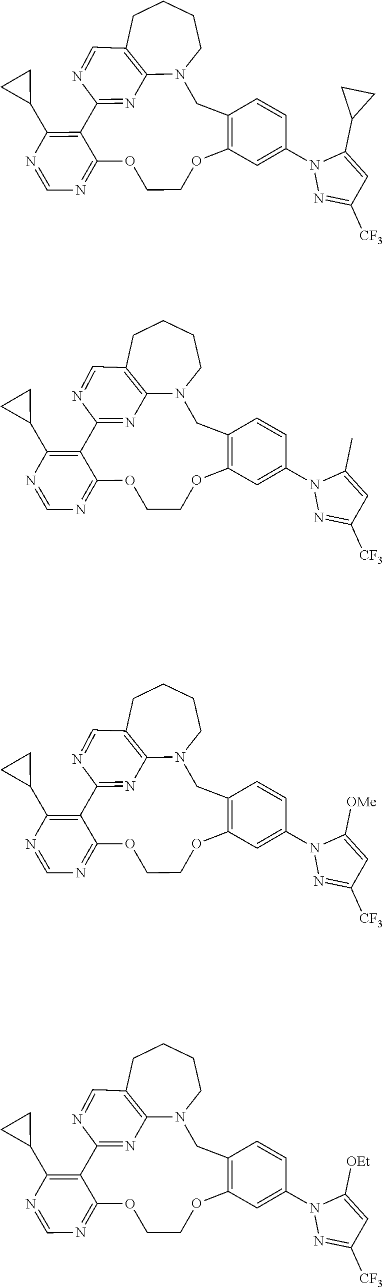 [SMILES: FC(F)(F)c1cc(C2CC2)n(-c2ccc3c(c2)OCCOc2ncnc(C4CC4)c2-c2ncc4c(n2)N(CCCC4)C3)n1 ; Cc1cc(C(F)(F)F)nn1-c1ccc2c(c1)OCCOc1ncnc(C3CC3)c1-c1ncc3c(n1)N(CCCC3)C2 ; COc1cc(C(F)(F)F)nn1-c1ccc2c(c1)OCCOc1ncnc(C3CC3)c1-c1ncc3c(n1)N(CCCC3)C2 ; CCOc1cc(C(F)(F)F)nn1-c1ccc2c(c1)OCCOc1ncnc(C3CC3)c1-c1ncc3c(n1)N(CCCC3)C2]

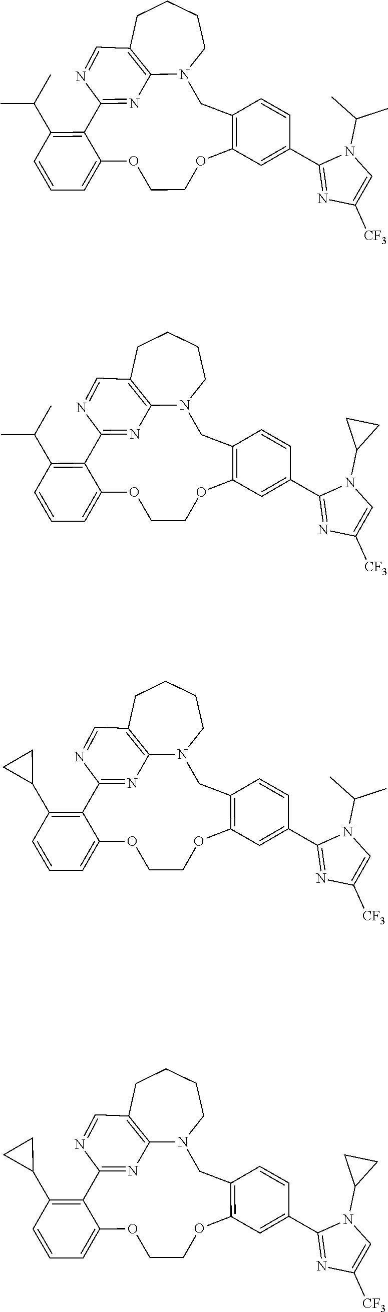

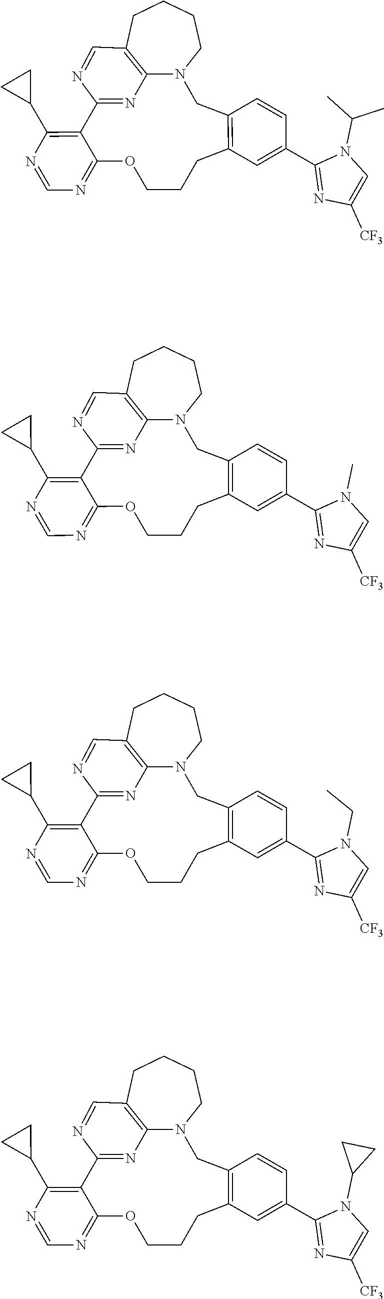 [SMILES: CC(C)n1cc(C(F)(F)F)nc1-c1ccc2c(c1)CCCOc1ncnc(C3CC3)c1-c1ncc3c(n1)N(CCCC3)C2 ; Cn1cc(C(F)(F)F)nc1-c1ccc2c(c1)CCCOc1ncnc(C3CC3)c1-c1ncc3c(n1)N(CCCC3)C2 ; CCn1cc(C(F)(F)F)nc1-c1ccc2c(c1)CCCOc1ncnc(C3CC3)c1-c1ncc3c(n1)N(CCCC3)C2 ; FC(F)(F)c1cn(C2CC2)c(-c2ccc3c(c2)CCCOc2ncnc(C4CC4)c2-c2ncc4c(n2)N(CCCC4)C3)n1]

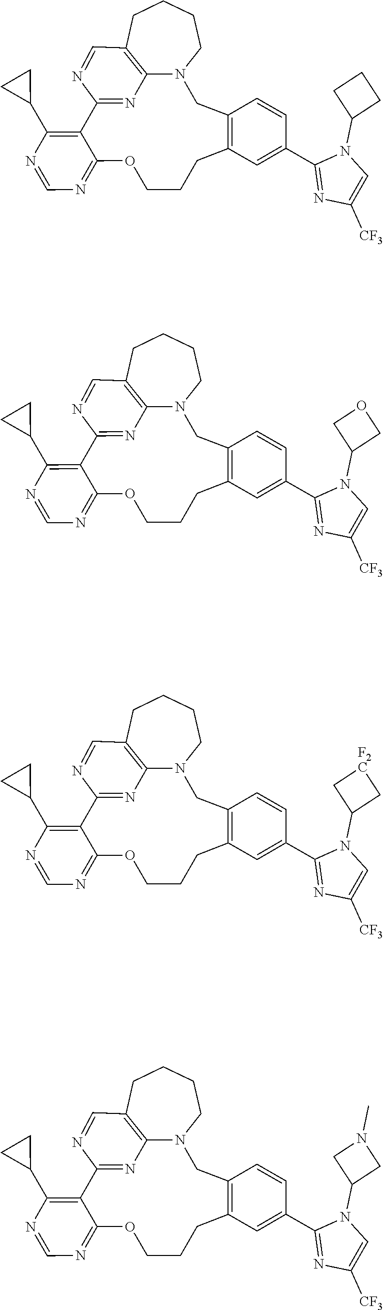 [SMILES: FC(F)(F)c1cn(C2CCC2)c(-c2ccc3c(c2)CCCOc2ncnc(C4CC4)c2-c2ncc4c(n2)N(CCCC4)C3)n1 ; FC(F)(F)c1cn(C2COC2)c(-c2ccc3c(c2)CCCOc2ncnc(C4CC4)c2-c2ncc4c(n2)N(CCCC4)C3)n1 ; FC1(F)CC(n2cc(C(F)(F)F)nc2-c2ccc3c(c2)CCCOc2ncnc(C4CC4)c2-c2ncc4c(n2)N(CCCC4)C3)C1 ; CN1CC(n2cc(C(F)(F)F)nc2-c2ccc3c(c2)CCCOc2ncnc(C4CC4)c2-c2ncc4c(n2)N(CCCC4)C3)C1]

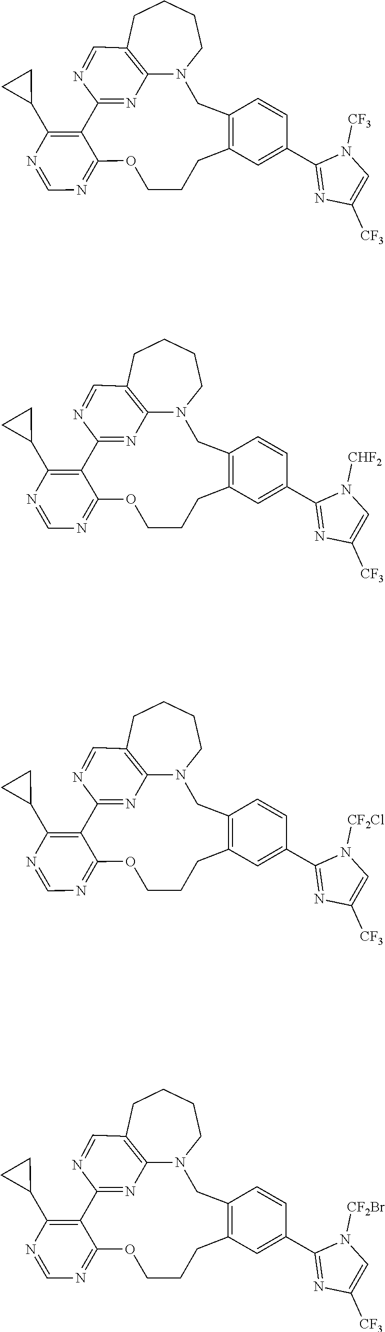

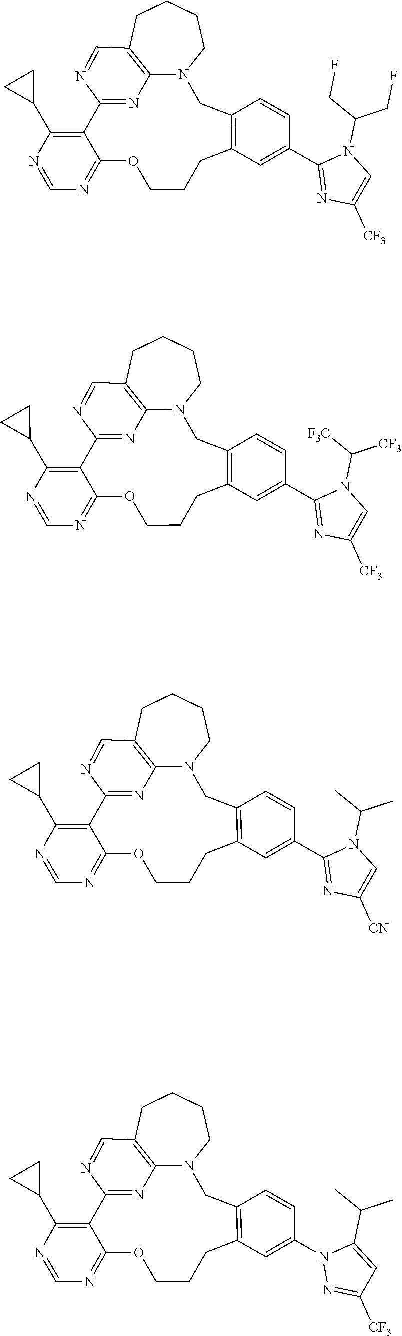 [SMILES: FCC(CF)n1cc(C(F)(F)F)nc1-c1ccc2c(c1)CCCOc1ncnc(C3CC3)c1-c1ncc3c(n1)N(CCCC3)C2 ; FC(F)(F)c1cn(C(C(F)(F)F)C(F)(F)F)c(-c2ccc3c(c2)CCCOc2ncnc(C4CC4)c2-c2ncc4c(n2)N(CCCC4)C3)n1 ; CC(C)n1cc(C#N)nc1-c1ccc2c(c1)CCCOc1ncnc(C3CC3)c1-c1ncc3c(n1)N(CCCC3)C2 ; CC(C)c1cc(C(F)(F)F)nn1-c1ccc2c(c1)CCCOc1ncnc(C3CC3)c1-c1ncc3c(n1)N(CCCC3)C2]

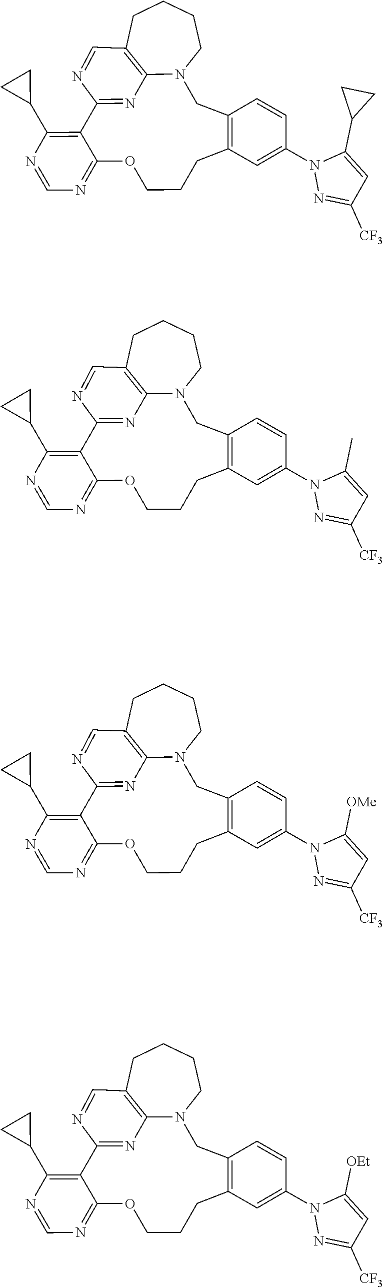 [SMILES: FC(F)(F)c1cc(C2CC2)n(-c2ccc3c(c2)CCCOc2ncnc(C4CC4)c2-c2ncc4c(n2)N(CCCC4)C3)n1 ; Cc1cc(C(F)(F)F)nn1-c1ccc2c(c1)CCCOc1ncnc(C3CC3)c1-c1ncc3c(n1)N(CCCC3)C2 ; COc1cc(C(F)(F)F)nn1-c1ccc2c(c1)CCCOc1ncnc(C3CC3)c1-c1ncc3c(n1)N(CCCC3)C2 ; CCOc1cc(C(F)(F)F)nn1-c1ccc2c(c1)CCCOc1ncnc(C3CC3)c1-c1ncc3c(n1)N(CCCC3)C2]

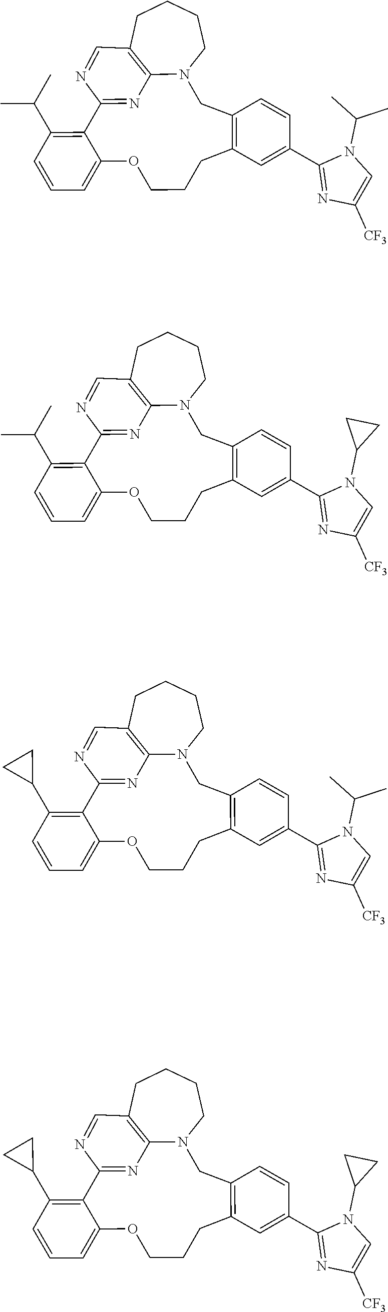

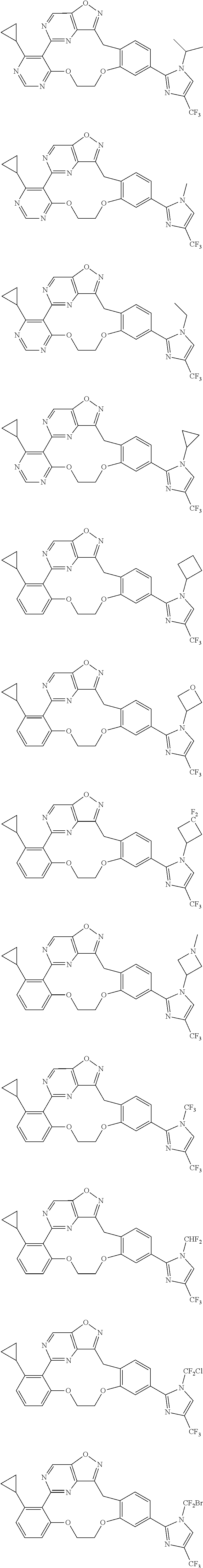

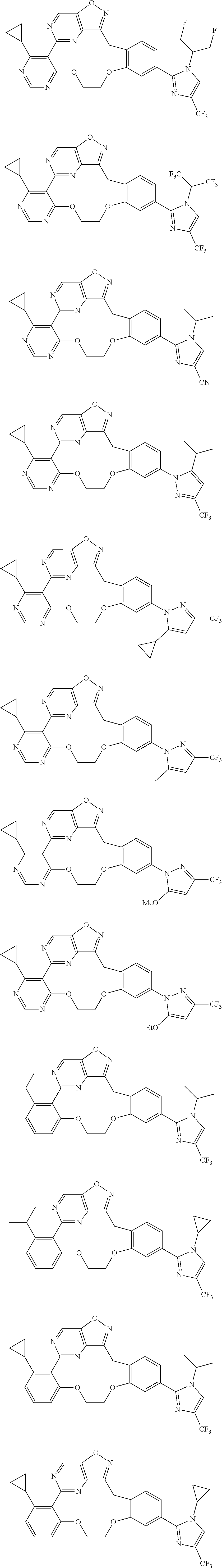

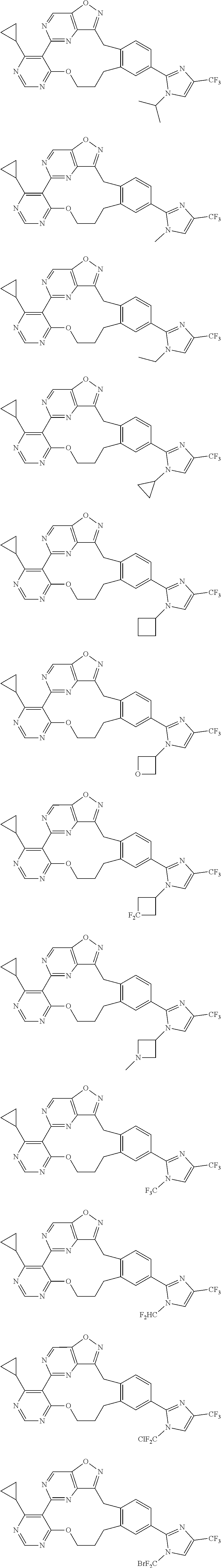

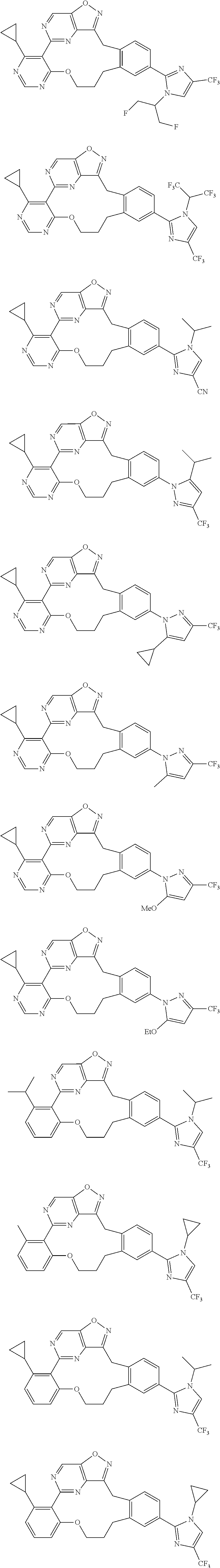

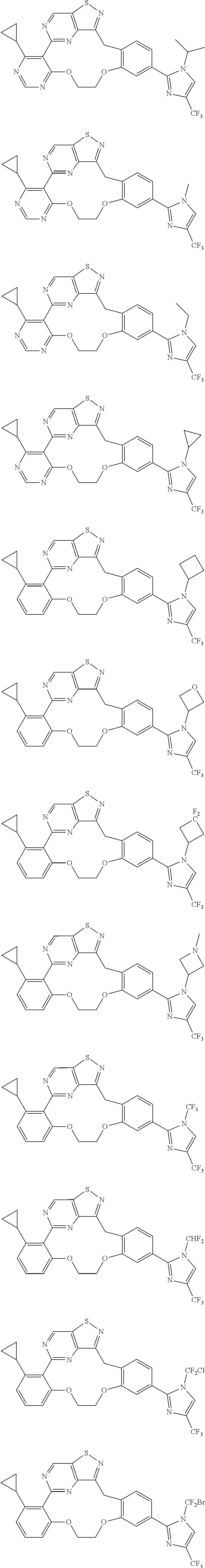

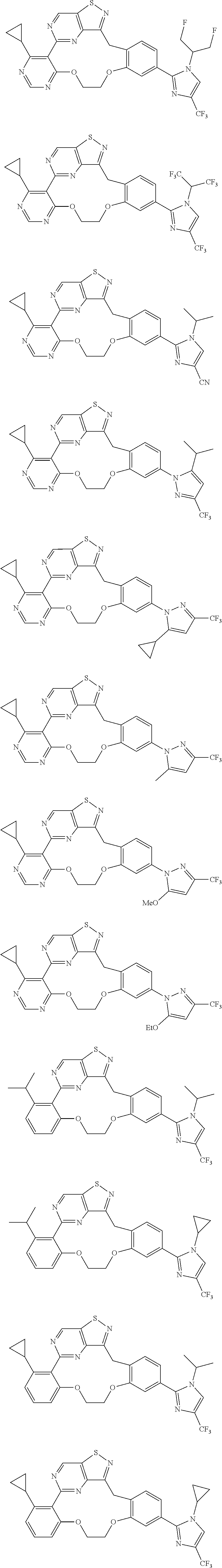

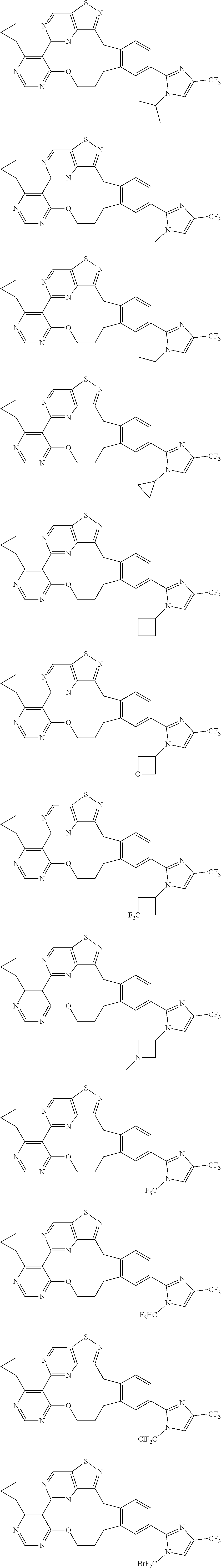

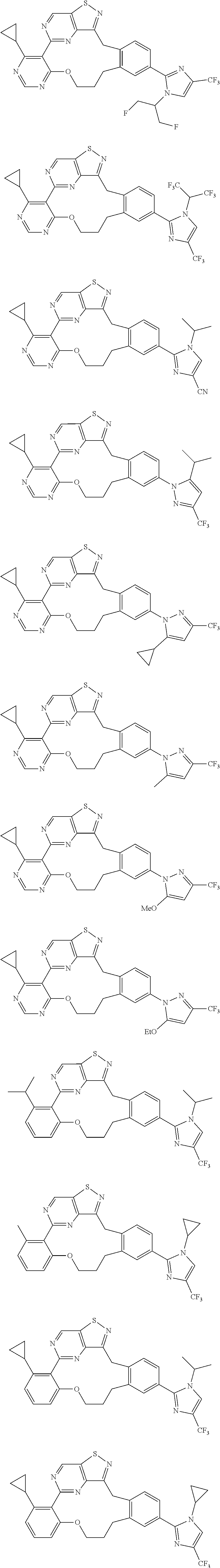

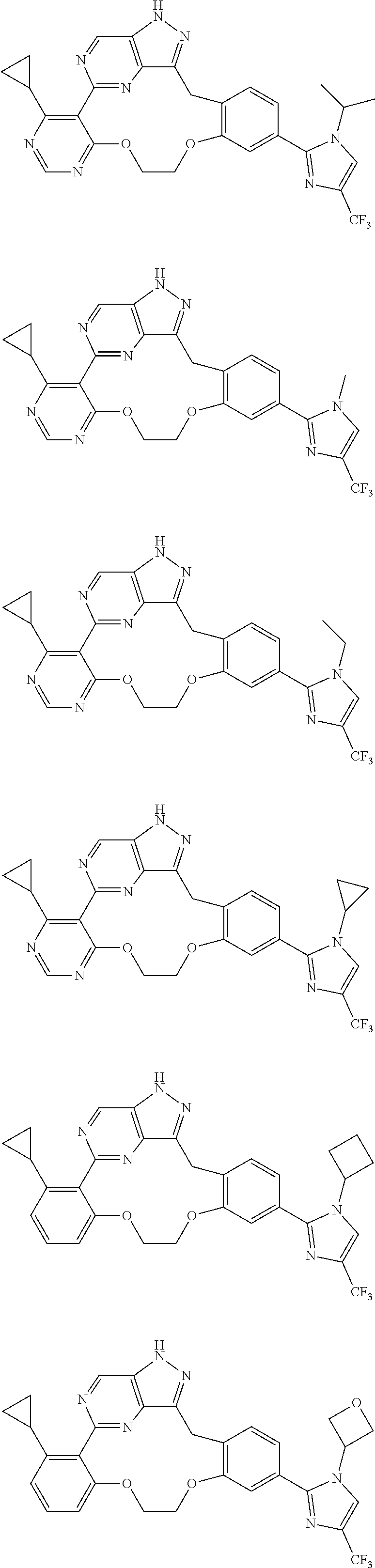 [SMILES: CC(C)n1cc(C(F)(F)F)nc1-c1ccc2c(c1)OCCOc1ncnc(C3CC3)c1-c1ncc3[nH]nc(c3n1)C2 ; Cn1cc(C(F)(F)F)nc1-c1ccc2c(c1)OCCOc1ncnc(C3CC3)c1-c1ncc3[nH]nc(c3n1)C2 ; CCn1cc(C(F)(F)F)nc1-c1ccc2c(c1)OCCOc1ncnc(C3CC3)c1-c1ncc3[nH]nc(c3n1)C2 ; FC(F)(F)c1cn(C2CC2)c(-c2ccc3c(c2)OCCOc2ncnc(C4CC4)c2-c2ncc4[nH]nc(c4n2)C3)n1 ; FC(F)(F)c1cn(C2CCC2)c(-c2ccc3c(c2)OCCOc2cccc(C4CC4)c2-c2ncc4[nH]nc(c4n2)C3)n1 ; FC(F)(F)c1cn(C2COC2)c(-c2ccc3c(c2)OCCOc2cccc(C4CC4)c2-c2ncc4[nH]nc(c4n2)C3)n1]

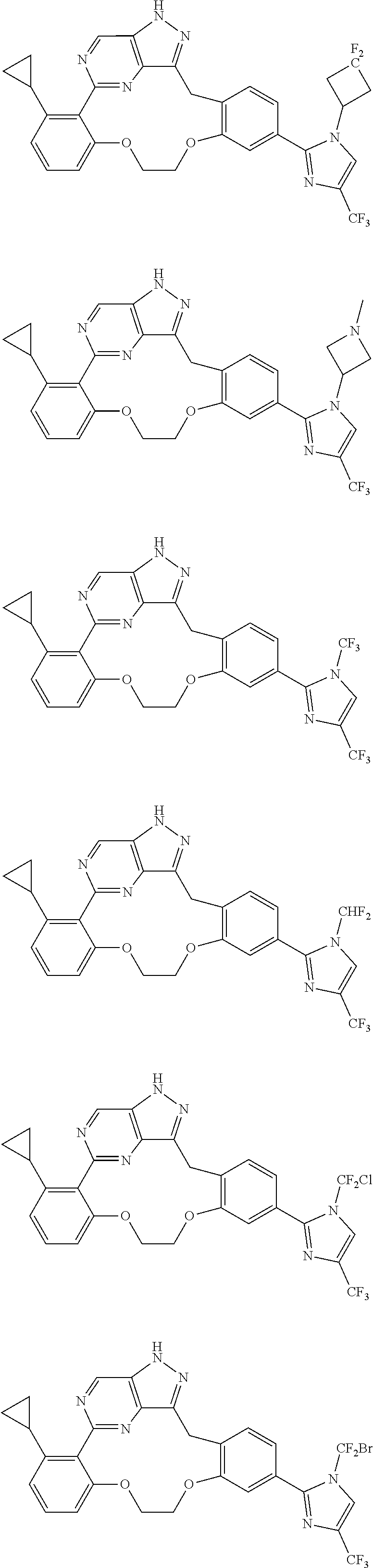

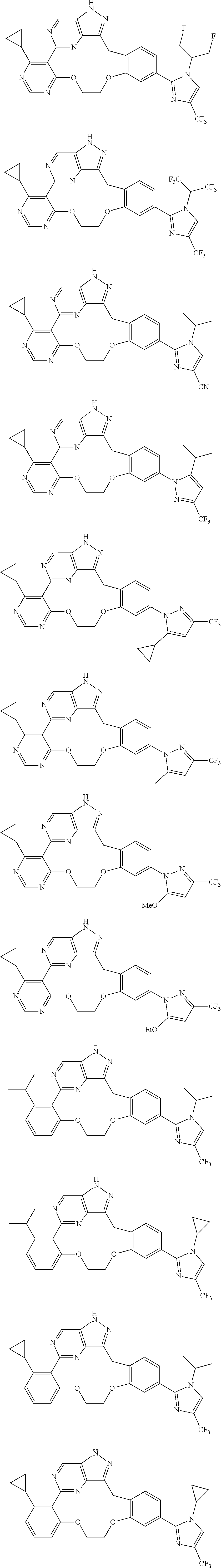

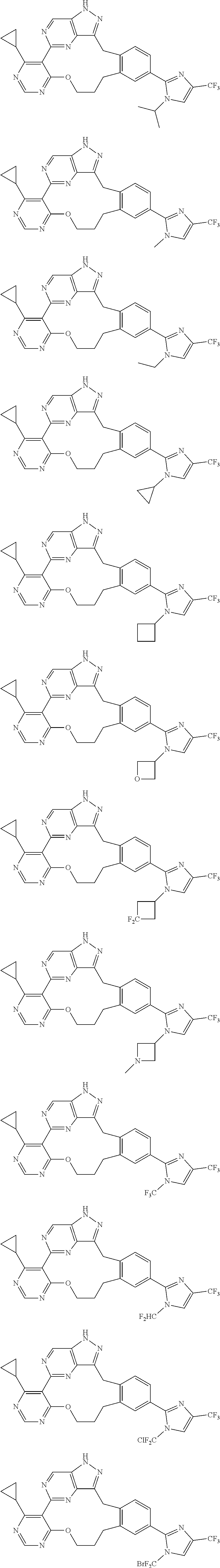

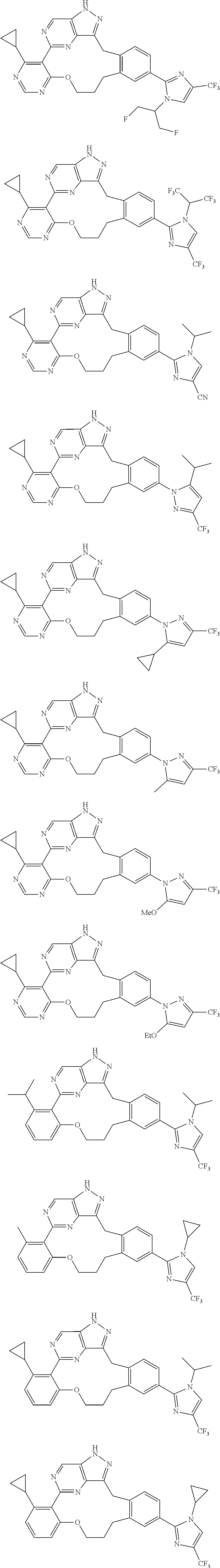

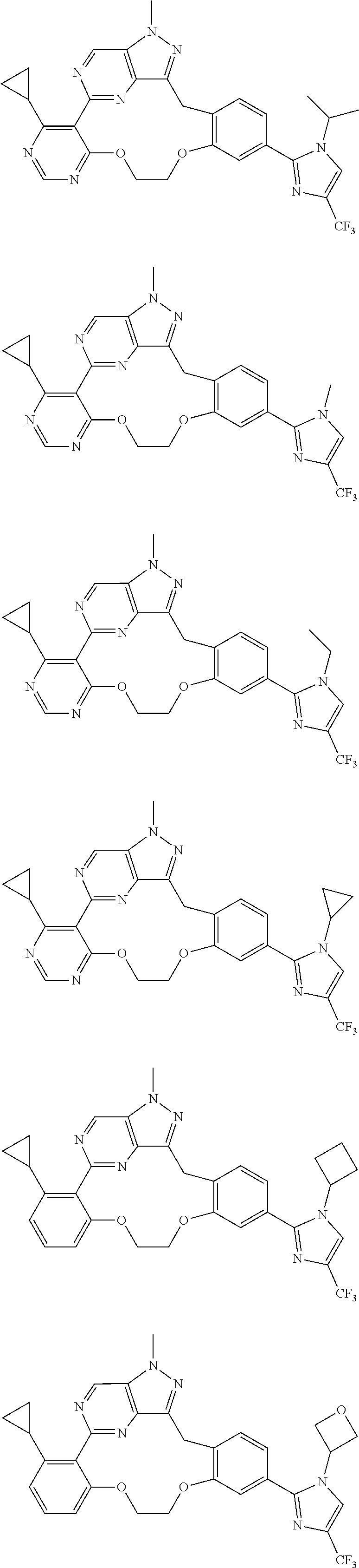 [SMILES: CC(C)n1cc(C(F)(F)F)nc1-c1ccc2c(c1)OCCOc1ncnc(C3CC3)c1-c1ncc3c(n1)c(nn3C)C2 ; Cn1cc(C(F)(F)F)nc1-c1ccc2c(c1)OCCOc1ncnc(C3CC3)c1-c1ncc3c(n1)c(nn3C)C2 ; CCn1cc(C(F)(F)F)nc1-c1ccc2c(c1)OCCOc1ncnc(C3CC3)c1-c1ncc3c(n1)c(nn3C)C2 ; Cn1nc2c3nc(ncc31)-c1c(ncnc1C1CC1)OCCOc1cc(-c3nc(C(F)(F)F)cn3C3CC3)ccc1C2 ; Cn1nc2c3nc(ncc31)-c1c(cccc1C1CC1)OCCOc1cc(-c3nc(C(F)(F)F)cn3C3CCC3)ccc1C2 ; Cn1nc2c3nc(ncc31)-c1c(cccc1C1CC1)OCCOc1cc(-c3nc(C(F)(F)F)cn3C3COC3)ccc1C2]

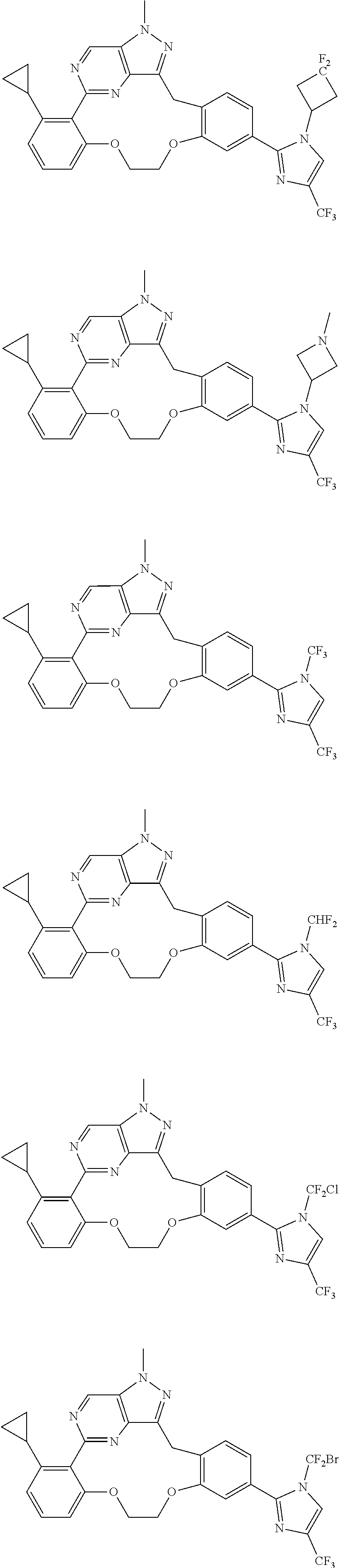

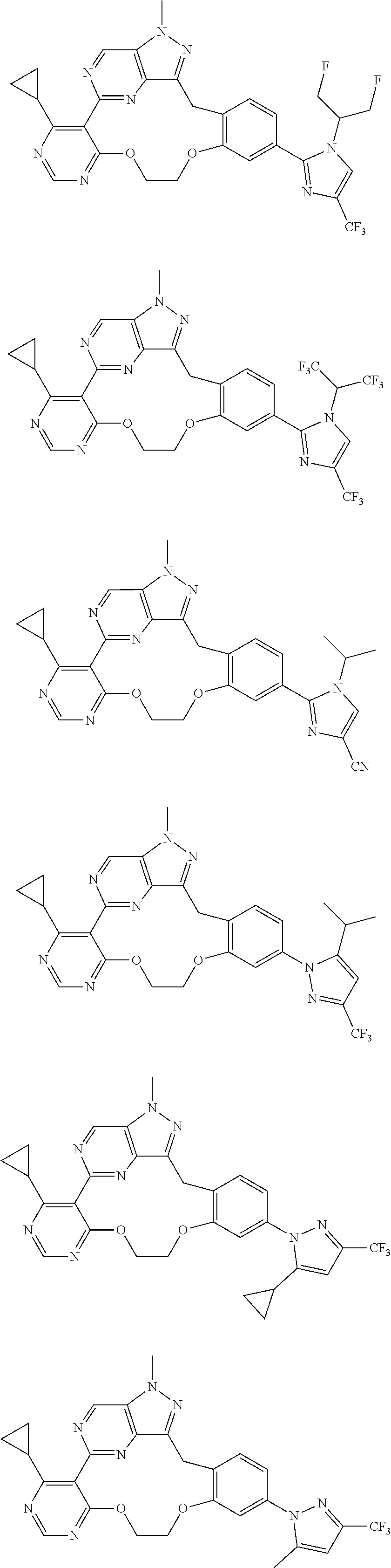 [SMILES: Cn1nc2c3nc(ncc31)-c1c(ncnc1C1CC1)OCCOc1cc(-c3nc(C(F)(F)F)cn3C(CF)CF)ccc1C2 ; Cn1nc2c3nc(ncc31)-c1c(ncnc1C1CC1)OCCOc1cc(-c3nc(C(F)(F)F)cn3C(C(F)(F)F)C(F)(F)F)ccc1C2 ; CC(C)n1cc(C#N)nc1-c1ccc2c(c1)OCCOc1ncnc(C3CC3)c1-c1ncc3c(n1)c(nn3C)C2 ; CC(C)c1cc(C(F)(F)F)nn1-c1ccc2c(c1)OCCOc1ncnc(C3CC3)c1-c1ncc3c(n1)c(nn3C)C2 ; Cn1nc2c3nc(ncc31)-c1c(ncnc1C1CC1)OCCOc1cc(-n3nc(C(F)(F)F)cc3C3CC3)ccc1C2 ; Cc1cc(C(F)(F)F)nn1-c1ccc2c(c1)OCCOc1ncnc(C3CC3)c1-c1ncc3c(n1)c(nn3C)C2]

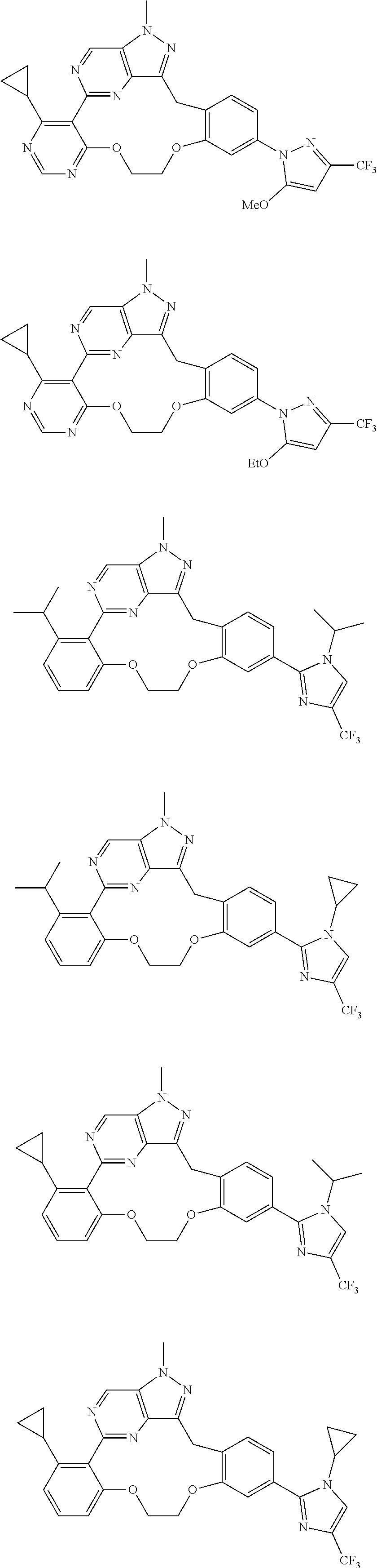

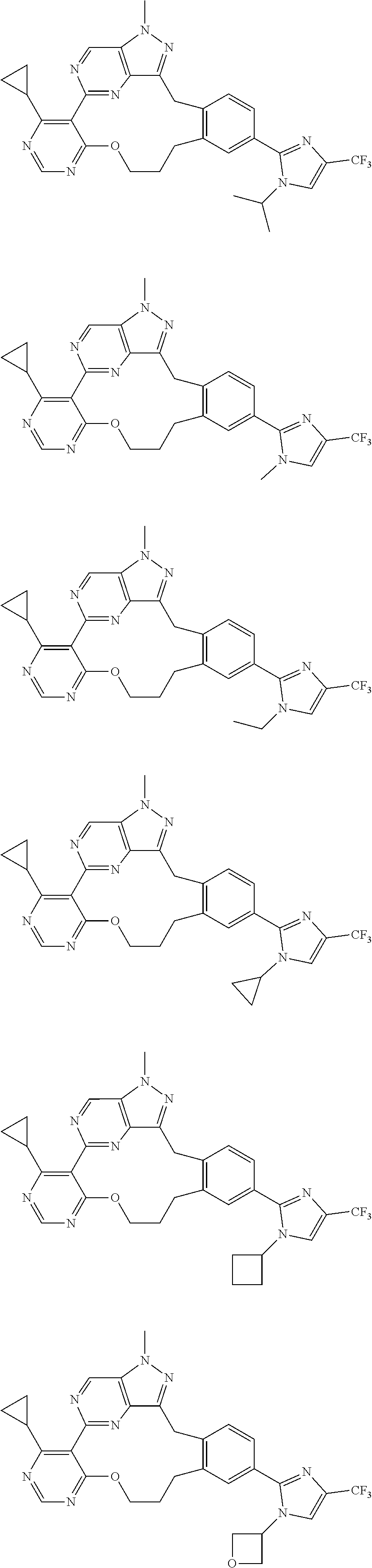 [SMILES: CC(C)n1cc(C(F)(F)F)nc1-c1ccc2c(c1)CCCOc1ncnc(C3CC3)c1-c1ncc3c(n1)c(nn3C)C2 ; Cn1cc(C(F)(F)F)nc1-c1ccc2c(c1)CCCOc1ncnc(C3CC3)c1-c1ncc3c(n1)c(nn3C)C2 ; CCn1cc(C(F)(F)F)nc1-c1ccc2c(c1)CCCOc1ncnc(C3CC3)c1-c1ncc3c(n1)c(nn3C)C2 ; Cn1nc2c3nc(ncc31)-c1c(ncnc1C1CC1)OCCCc1cc(-c3nc(C(F)(F)F)cn3C3CC3)ccc1C2 ; Cn1nc2c3nc(ncc31)-c1c(ncnc1C1CC1)OCCCc1cc(-c3nc(C(F)(F)F)cn3C3CCC3)ccc1C2 ; Cn1nc2c3nc(ncc31)-c1c(ncnc1C1CC1)OCCCc1cc(-c3nc(C(F)(F)F)cn3C3COC3)ccc1C2]

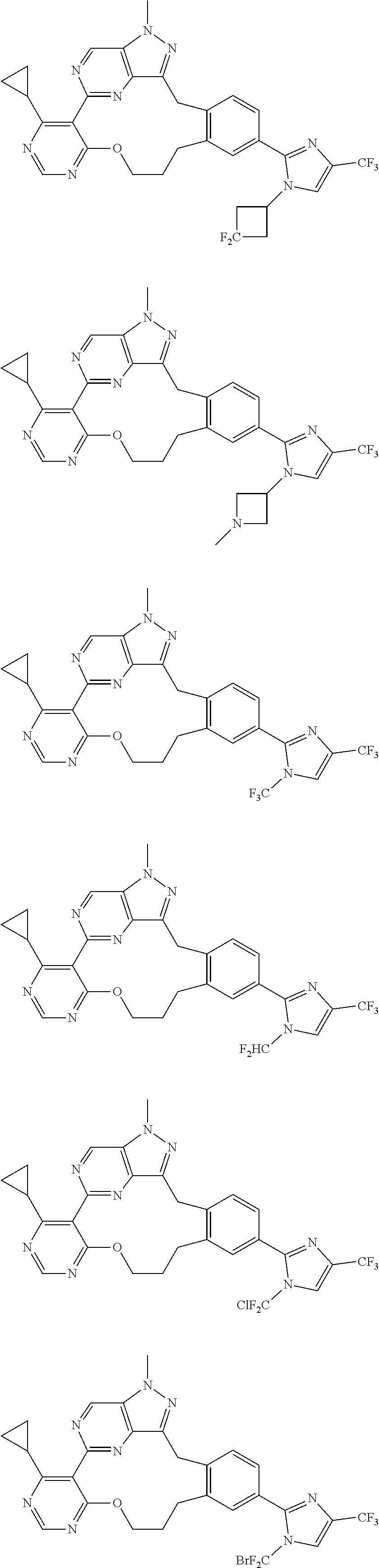

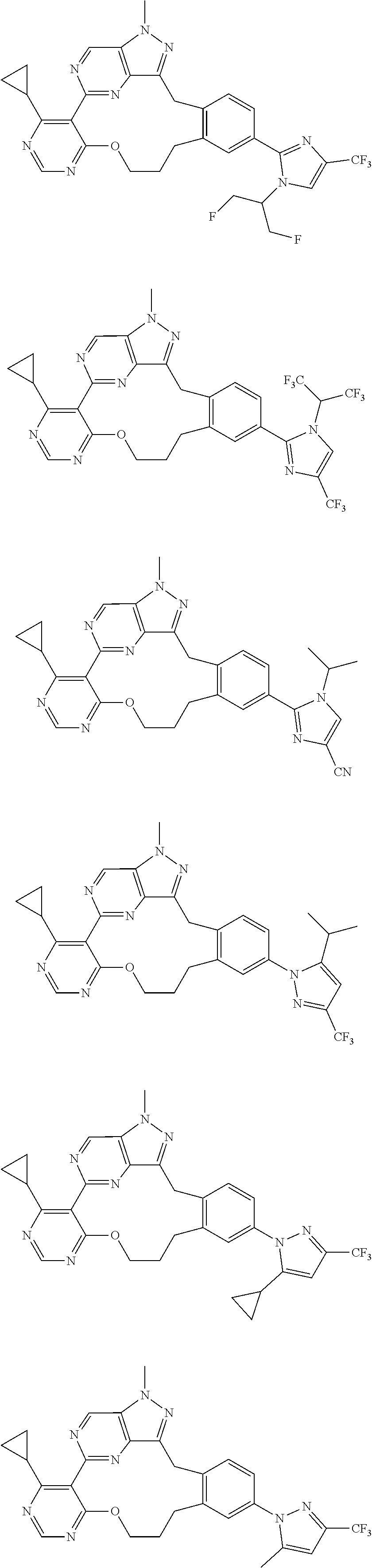 [SMILES: Cn1nc2c3nc(ncc31)-c1c(ncnc1C1CC1)OCCCc1cc(-c3nc(C(F)(F)F)cn3C(CF)CF)ccc1C2 ; Cn1nc2c3nc(ncc31)-c1c(ncnc1C1CC1)OCCCc1cc(-c3nc(C(F)(F)F)cn3C(C(F)(F)F)C(F)(F)F)ccc1C2 ; CC(C)n1cc(C#N)nc1-c1ccc2c(c1)CCCOc1ncnc(C3CC3)c1-c1ncc3c(n1)c(nn3C)C2 ; CC(C)c1cc(C(F)(F)F)nn1-c1ccc2c(c1)CCCOc1ncnc(C3CC3)c1-c1ncc3c(n1)c(nn3C)C2 ; Cn1nc2c3nc(ncc31)-c1c(ncnc1C1CC1)OCCCc1cc(-n3nc(C(F)(F)F)cc3C3CC3)ccc1C2 ; Cc1cc(C(F)(F)F)nn1-c1ccc2c(c1)CCCOc1ncnc(C3CC3)c1-c1ncc3c(n1)c(nn3C)C2]

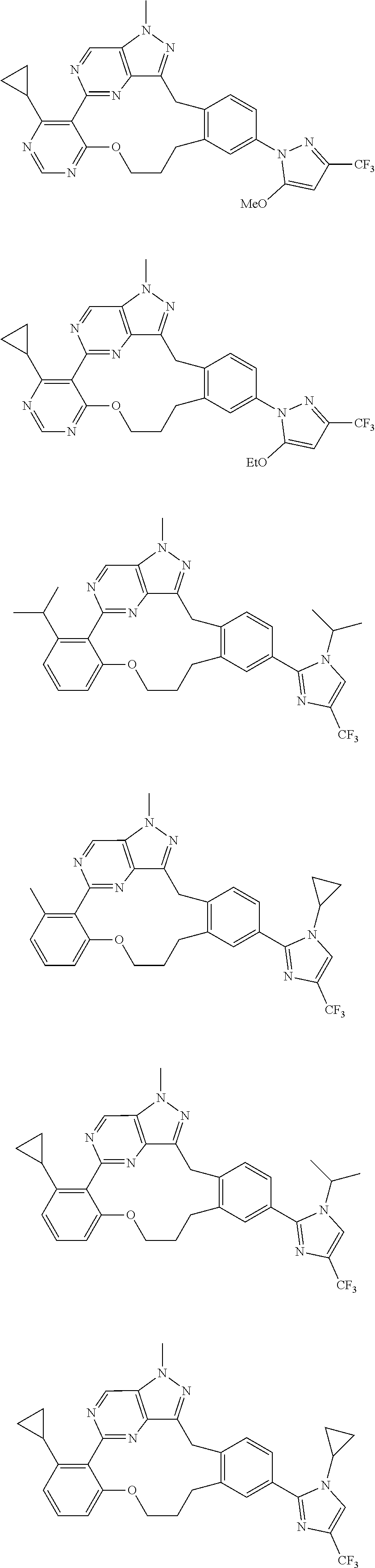 [SMILES: COc1cc(C(F)(F)F)nn1-c1ccc2c(c1)CCCOc1ncnc(C3CC3)c1-c1ncc3c(n1)c(nn3C)C2 ; CCOc1cc(C(F)(F)F)nn1-c1ccc2c(c1)CCCOc1ncnc(C3CC3)c1-c1ncc3c(n1)c(nn3C)C2 ; CC(C)c1cccc2c1-c1ncc3c(n1)c(nn3C)Cc1ccc(-c3nc(C(F)(F)F)cn3C(C)C)cc1CCCO2 ; Cc1cccc2c1-c1ncc3c(n1)c(nn3C)Cc1ccc(-c3nc(C(F)(F)F)cn3C3CC3)cc1CCCO2 ; CC(C)n1cc(C(F)(F)F)nc1-c1ccc2c(c1)CCCOc1cccc(C3CC3)c1-c1ncc3c(n1)c(nn3C)C2 ; Cn1nc2c3nc(ncc31)-c1c(cccc1C1CC1)OCCCc1cc(-c3nc(C(F)(F)F)cn3C3CC3)ccc1C2]

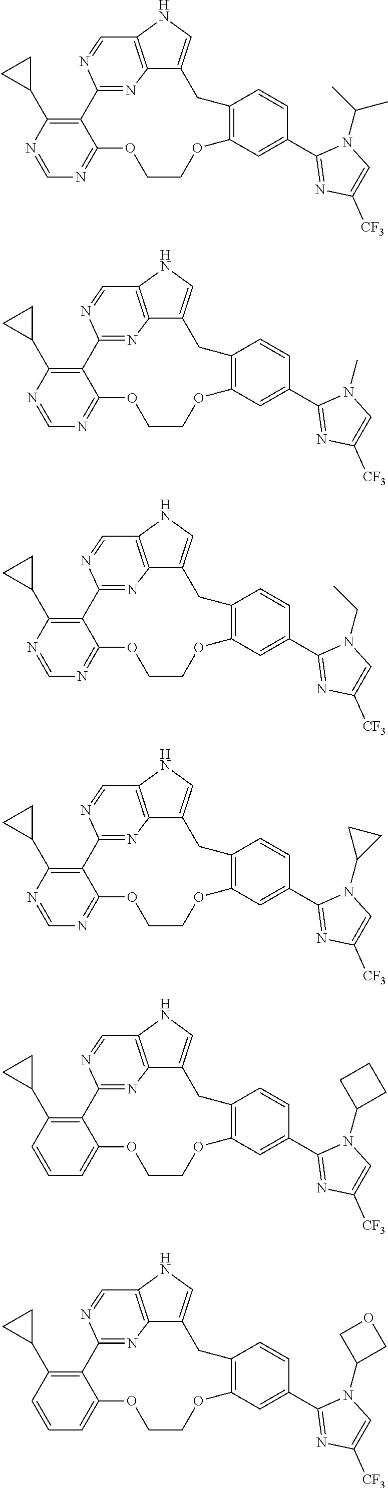 [SMILES: CC(C)n1cc(C(F)(F)F)nc1-c1ccc2c(c1)OCCOc1ncnc(C3CC3)c1-c1ncc3[nH]cc(c3n1)C2 ; Cn1cc(C(F)(F)F)nc1-c1ccc2c(c1)OCCOc1ncnc(C3CC3)c1-c1ncc3[nH]cc(c3n1)C2 ; CCn1cc(C(F)(F)F)nc1-c1ccc2c(c1)OCCOc1ncnc(C3CC3)c1-c1ncc3[nH]cc(c3n1)C2 ; FC(F)(F)c1cn(C2CC2)c(-c2ccc3c(c2)OCCOc2ncnc(C4CC4)c2-c2ncc4[nH]cc(c4n2)C3)n1 ; FC(F)(F)c1cn(C2CCC2)c(-c2ccc3c(c2)OCCOc2cccc(C4CC4)c2-c2ncc4[nH]cc(c4n2)C3)n1 ; FC(F)(F)c1cn(C2COC2)c(-c2ccc3c(c2)OCCOc2cccc(C4CC4)c2-c2ncc4[nH]cc(c4n2)C3)n1]

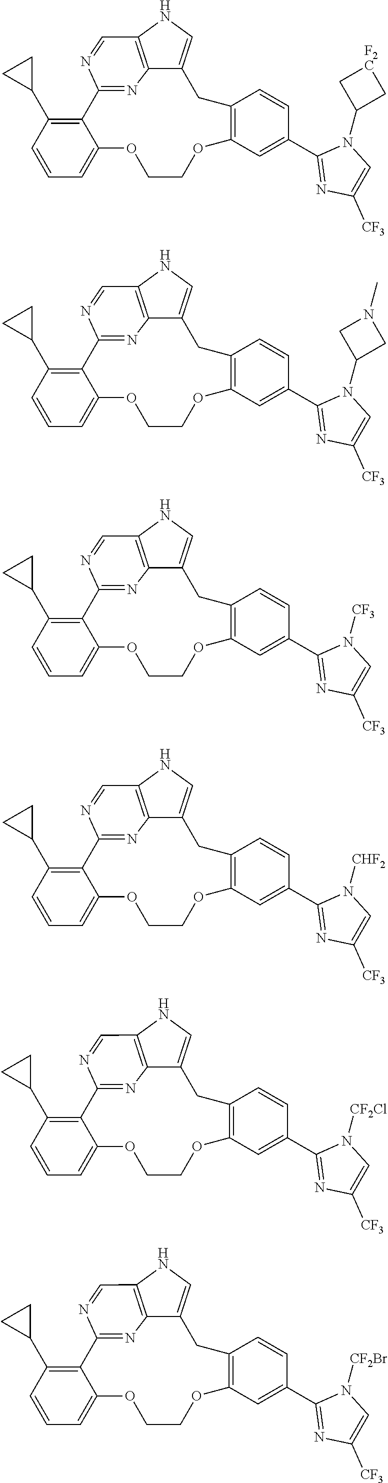

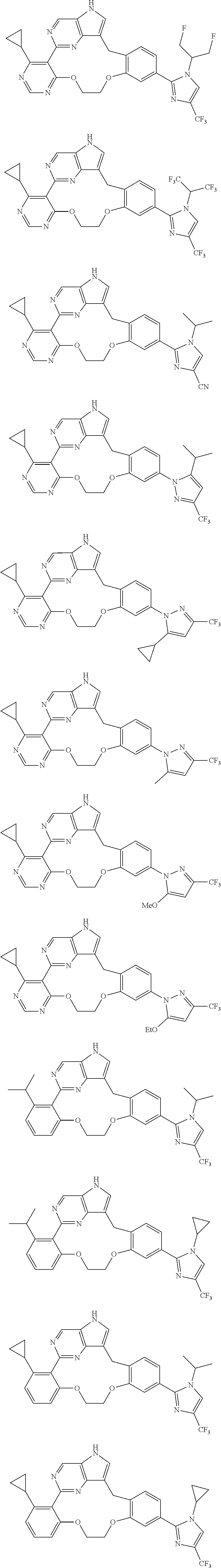

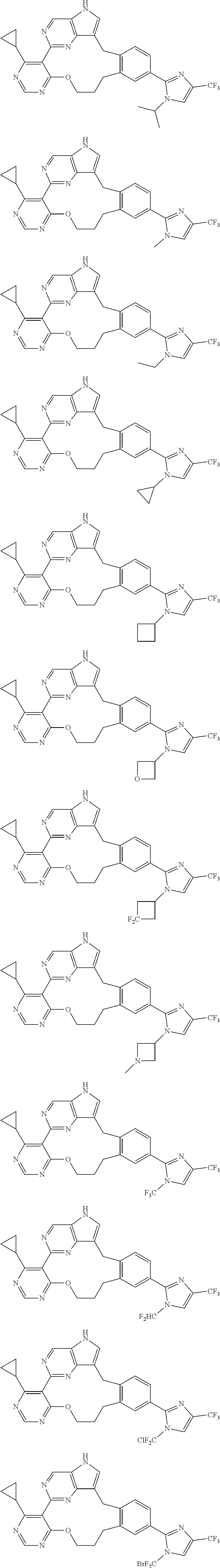

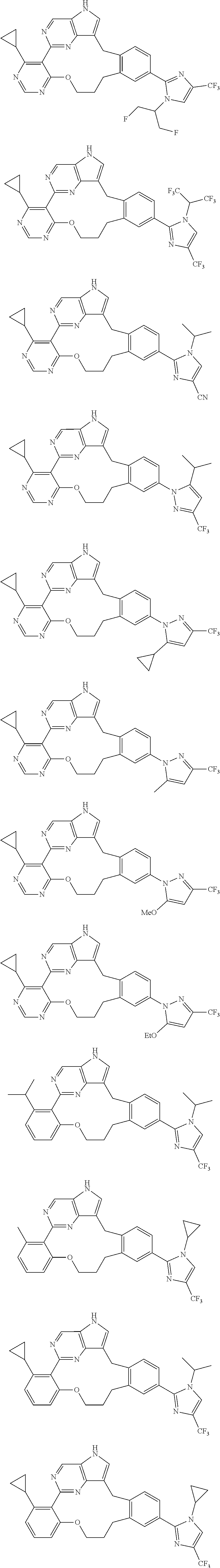

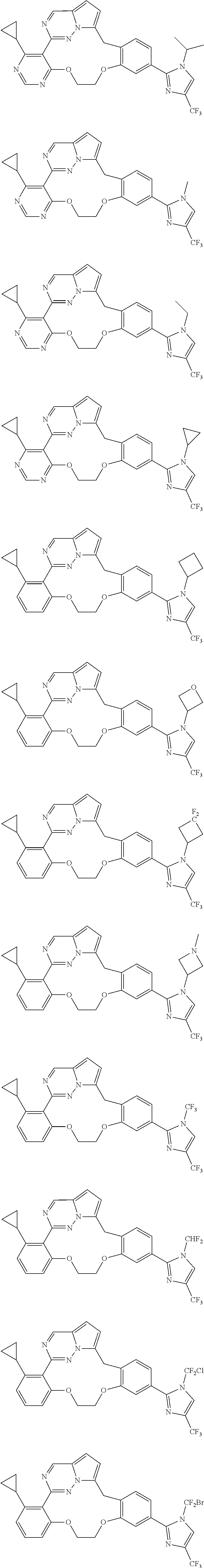

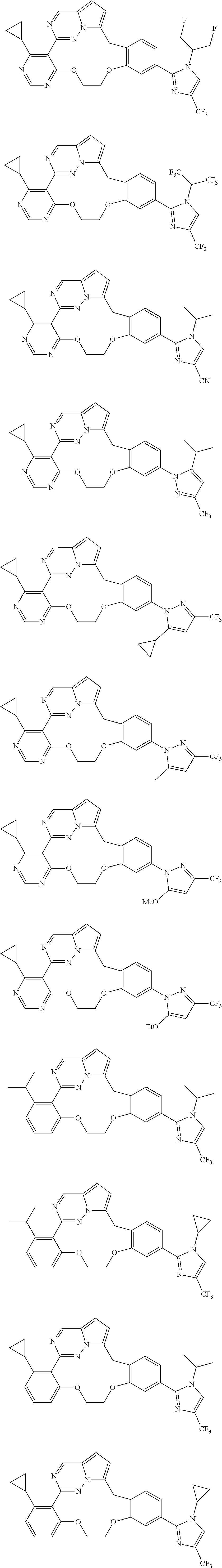

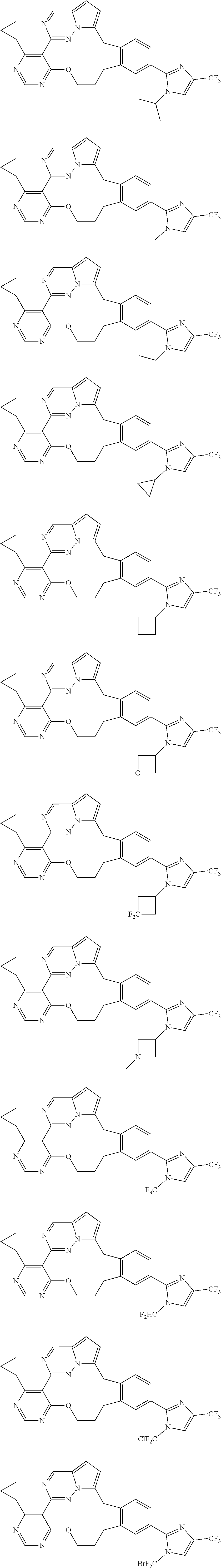

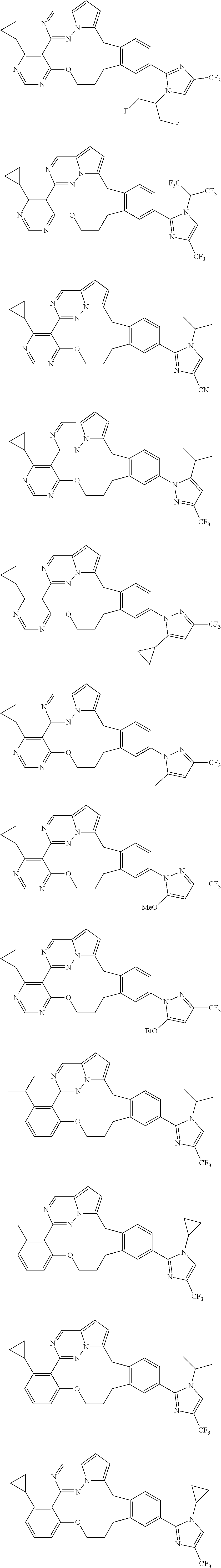

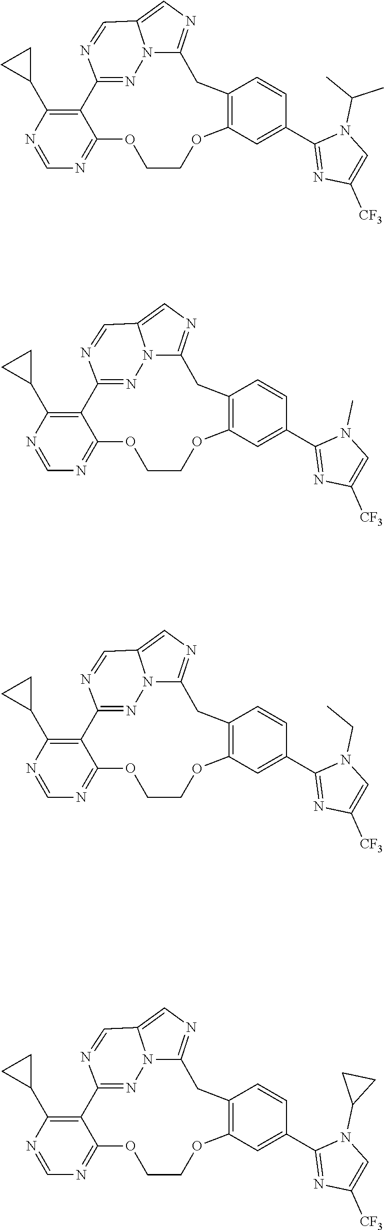 [SMILES: CC(C)n1cc(C(F)(F)F)nc1-c1ccc2c(c1)OCCOc1ncnc(C3CC3)c1-c1ncc3cnc(n3n1)C2 ; Cn1cc(C(F)(F)F)nc1-c1ccc2c(c1)OCCOc1ncnc(C3CC3)c1-c1ncc3cnc(n3n1)C2 ; CCn1cc(C(F)(F)F)nc1-c1ccc2c(c1)OCCOc1ncnc(C3CC3)c1-c1ncc3cnc(n3n1)C2 ; FC(F)(F)c1cn(C2CC2)c(-c2ccc3c(c2)OCCOc2ncnc(C4CC4)c2-c2ncc4cnc(n4n2)C3)n1]

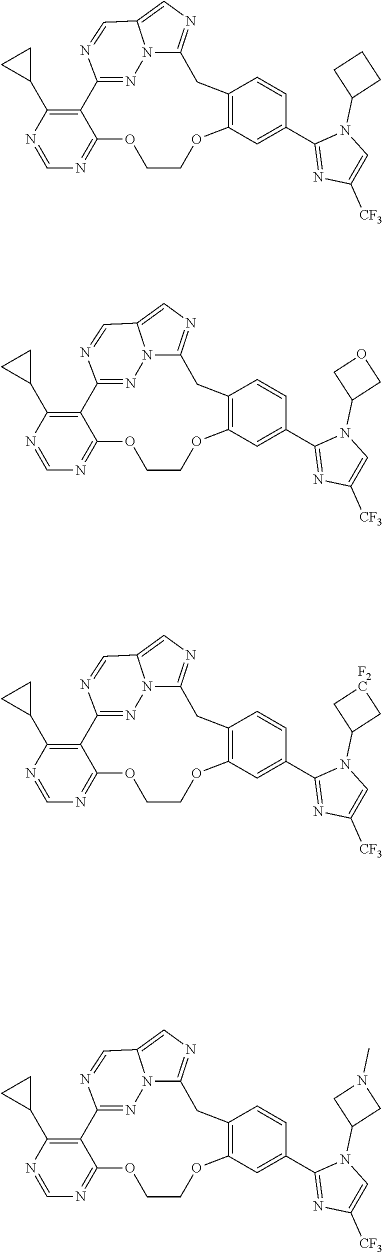 [SMILES: FC(F)(F)c1cn(C2CCC2)c(-c2ccc3c(c2)OCCOc2ncnc(C4CC4)c2-c2ncc4cnc(n4n2)C3)n1 ; FC(F)(F)c1cn(C2COC2)c(-c2ccc3c(c2)OCCOc2ncnc(C4CC4)c2-c2ncc4cnc(n4n2)C3)n1 ; FC1(F)CC(n2cc(C(F)(F)F)nc2-c2ccc3c(c2)OCCOc2ncnc(C4CC4)c2-c2ncc4cnc(n4n2)C3)C1 ; CN1CC(n2cc(C(F)(F)F)nc2-c2ccc3c(c2)OCCOc2ncnc(C4CC4)c2-c2ncc4cnc(n4n2)C3)C1]

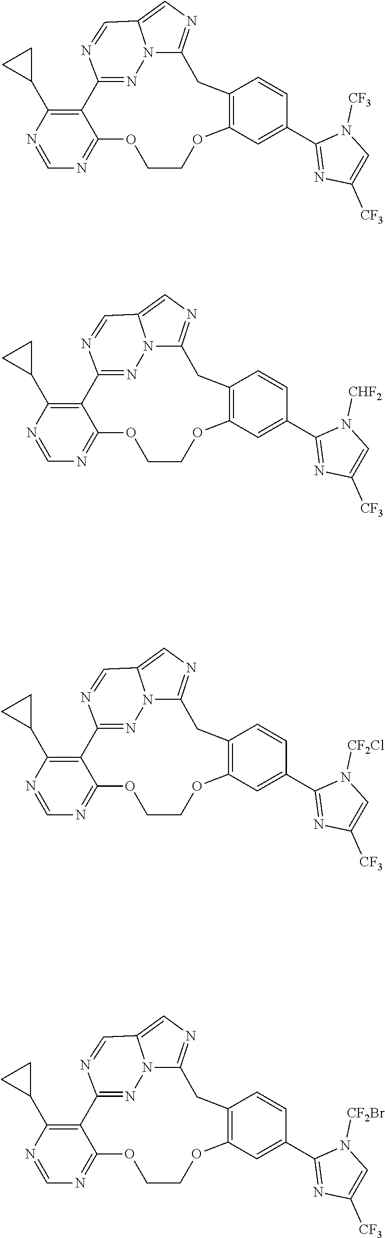

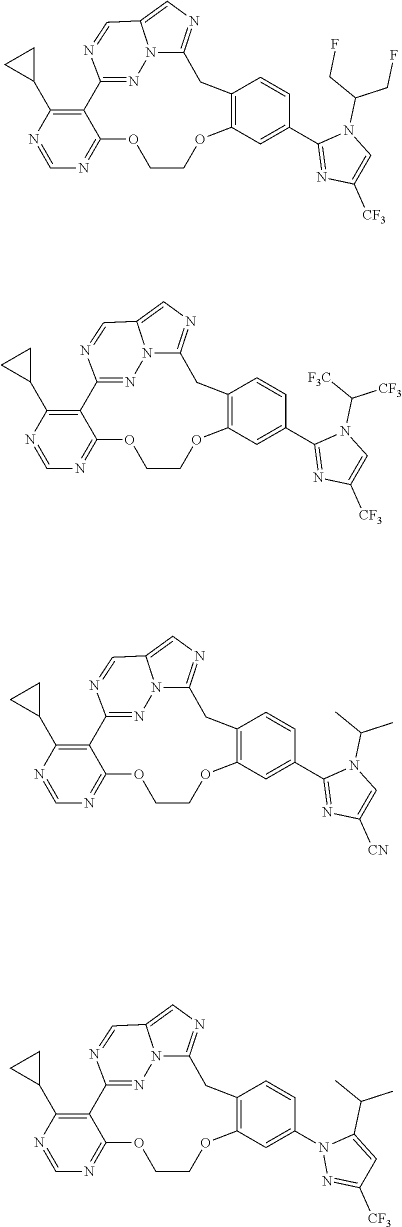 [SMILES: FCC(CF)n1cc(C(F)(F)F)nc1-c1ccc2c(c1)OCCOc1ncnc(C3CC3)c1-c1ncc3cnc(n3n1)C2 ; FC(F)(F)c1cn(C(C(F)(F)F)C(F)(F)F)c(-c2ccc3c(c2)OCCOc2ncnc(C4CC4)c2-c2ncc4cnc(n4n2)C3)n1 ; CC(C)n1cc(C#N)nc1-c1ccc2c(c1)OCCOc1ncnc(C3CC3)c1-c1ncc3cnc(n3n1)C2 ; CC(C)c1cc(C(F)(F)F)nn1-c1ccc2c(c1)OCCOc1ncnc(C3CC3)c1-c1ncc3cnc(n3n1)C2]

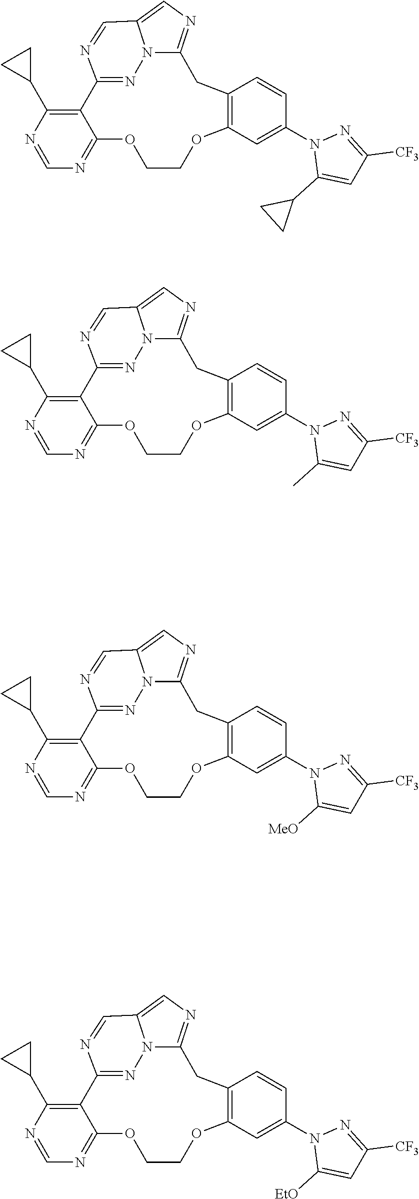 [SMILES: FC(F)(F)c1cc(C2CC2)n(-c2ccc3c(c2)OCCOc2ncnc(C4CC4)c2-c2ncc4cnc(n4n2)C3)n1 ; Cc1cc(C(F)(F)F)nn1-c1ccc2c(c1)OCCOc1ncnc(C3CC3)c1-c1ncc3cnc(n3n1)C2 ; COc1cc(C(F)(F)F)nn1-c1ccc2c(c1)OCCOc1ncnc(C3CC3)c1-c1ncc3cnc(n3n1)C2 ; CCOc1cc(C(F)(F)F)nn1-c1ccc2c(c1)OCCOc1ncnc(C3CC3)c1-c1ncc3cnc(n3n1)C2]

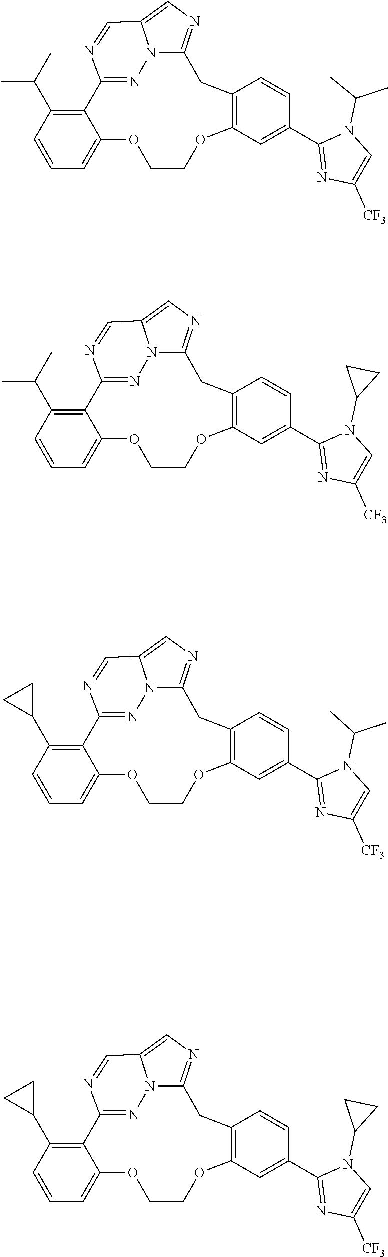

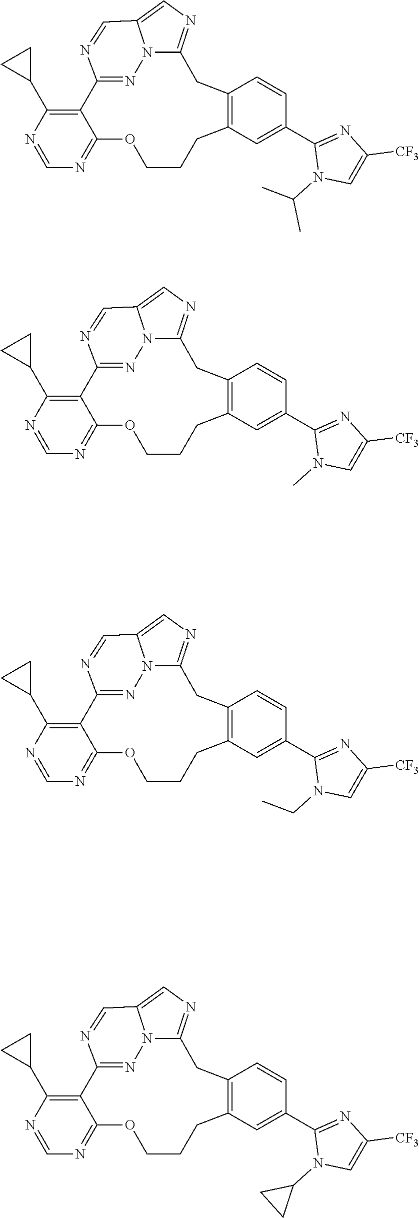 [SMILES: CC(C)n1cc(C(F)(F)F)nc1-c1ccc2c(c1)CCCOc1ncnc(C3CC3)c1-c1ncc3cnc(n3n1)C2 ; Cn1cc(C(F)(F)F)nc1-c1ccc2c(c1)CCCOc1ncnc(C3CC3)c1-c1ncc3cnc(n3n1)C2 ; CCn1cc(C(F)(F)F)nc1-c1ccc2c(c1)CCCOc1ncnc(C3CC3)c1-c1ncc3cnc(n3n1)C2 ; FC(F)(F)c1cn(C2CC2)c(-c2ccc3c(c2)CCCOc2ncnc(C4CC4)c2-c2ncc4cnc(n4n2)C3)n1]

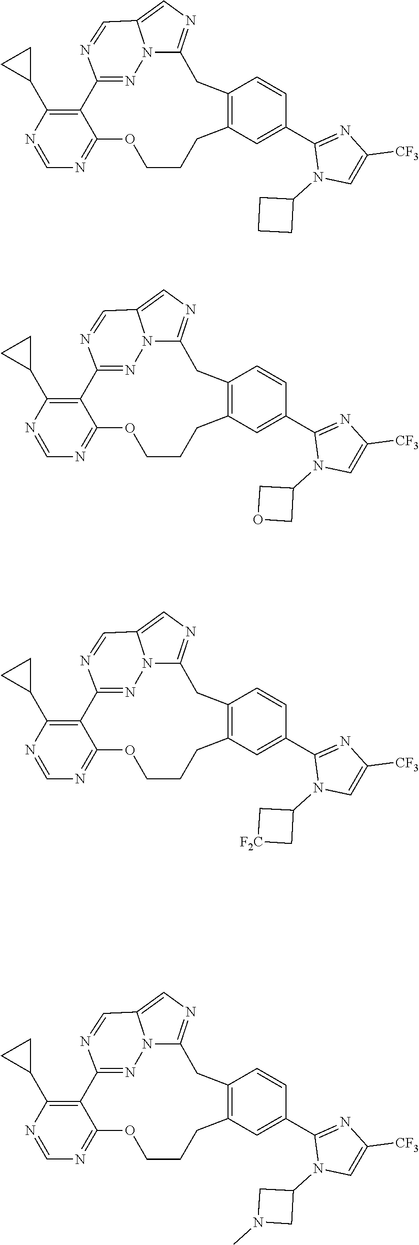 [SMILES: FC(F)(F)c1cn(C2CCC2)c(-c2ccc3c(c2)CCCOc2ncnc(C4CC4)c2-c2ncc4cnc(n4n2)C3)n1 ; FC(F)(F)c1cn(C2COC2)c(-c2ccc3c(c2)CCCOc2ncnc(C4CC4)c2-c2ncc4cnc(n4n2)C3)n1 ; FC1(F)CC(n2cc(C(F)(F)F)nc2-c2ccc3c(c2)CCCOc2ncnc(C4CC4)c2-c2ncc4cnc(n4n2)C3)C1 ; CN1CC(n2cc(C(F)(F)F)nc2-c2ccc3c(c2)CCCOc2ncnc(C4CC4)c2-c2ncc4cnc(n4n2)C3)C1]

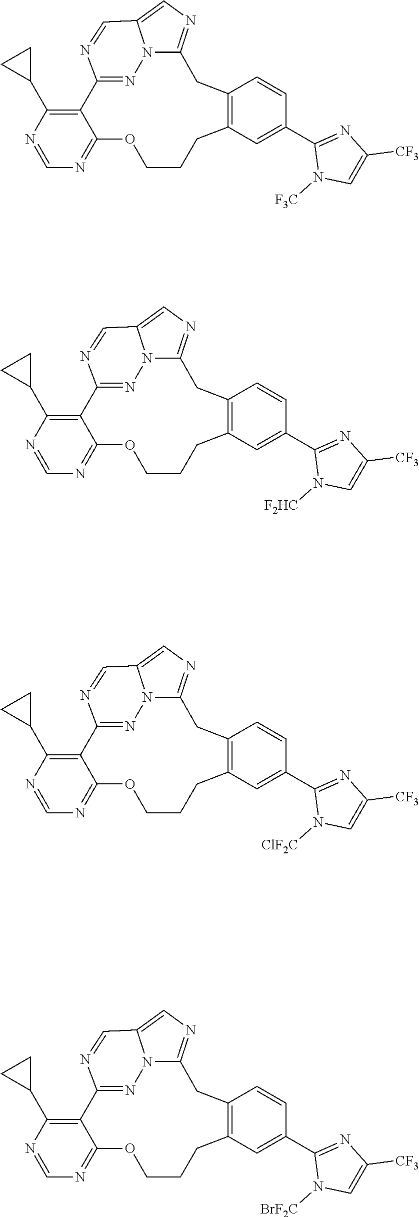

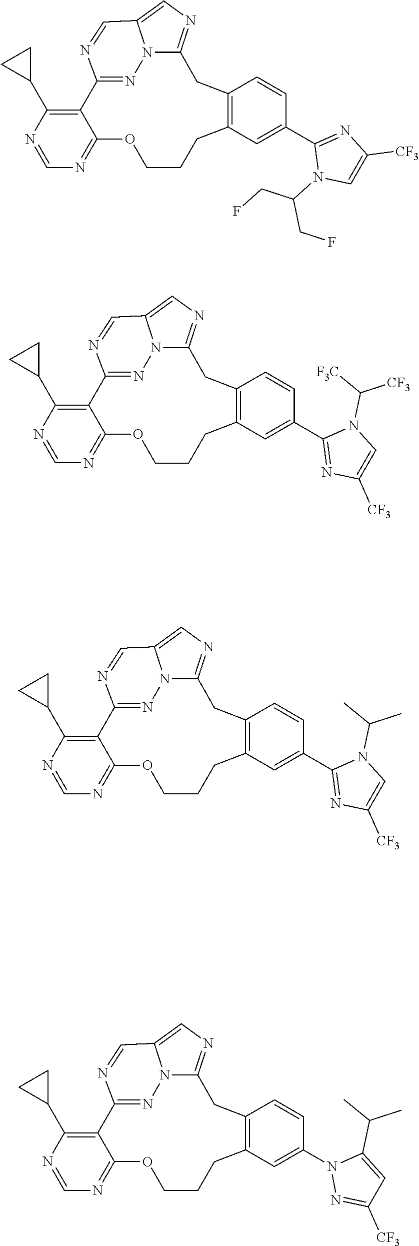 [SMILES: FCC(CF)n1cc(C(F)(F)F)nc1-c1ccc2c(c1)CCCOc1ncnc(C3CC3)c1-c1ncc3cnc(n3n1)C2 ; FC(F)(F)c1cn(C(C(F)(F)F)C(F)(F)F)c(-c2ccc3c(c2)CCCOc2ncnc(C4CC4)c2-c2ncc4cnc(n4n2)C3)n1 ; CC(C)n1cc(C(F)(F)F)nc1-c1ccc2c(c1)CCCOc1ncnc(C3CC3)c1-c1ncc3cnc(n3n1)C2 ; CC(C)c1cc(C(F)(F)F)nn1-c1ccc2c(c1)CCCOc1ncnc(C3CC3)c1-c1ncc3cnc(n3n1)C2]

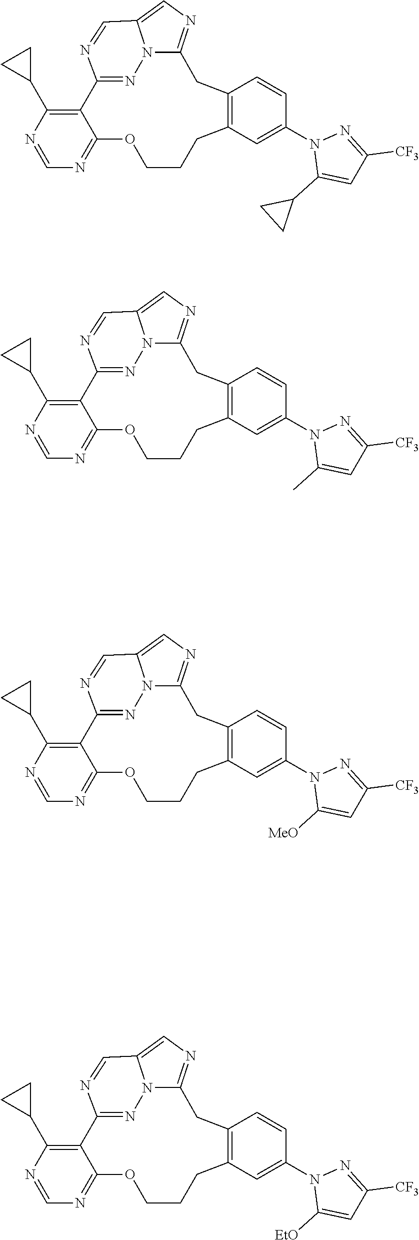 [SMILES: FC(F)(F)c1cc(C2CC2)n(-c2ccc3c(c2)CCCOc2ncnc(C4CC4)c2-c2ncc4cnc(n4n2)C3)n1 ; Cc1cc(C(F)(F)F)nn1-c1ccc2c(c1)CCCOc1ncnc(C3CC3)c1-c1ncc3cnc(n3n1)C2 ; COc1cc(C(F)(F)F)nn1-c1ccc2c(c1)CCCOc1ncnc(C3CC3)c1-c1ncc3cnc(n3n1)C2 ; CCOc1cc(C(F)(F)F)nn1-c1ccc2c(c1)CCCOc1ncnc(C3CC3)c1-c1ncc3cnc(n3n1)C2]

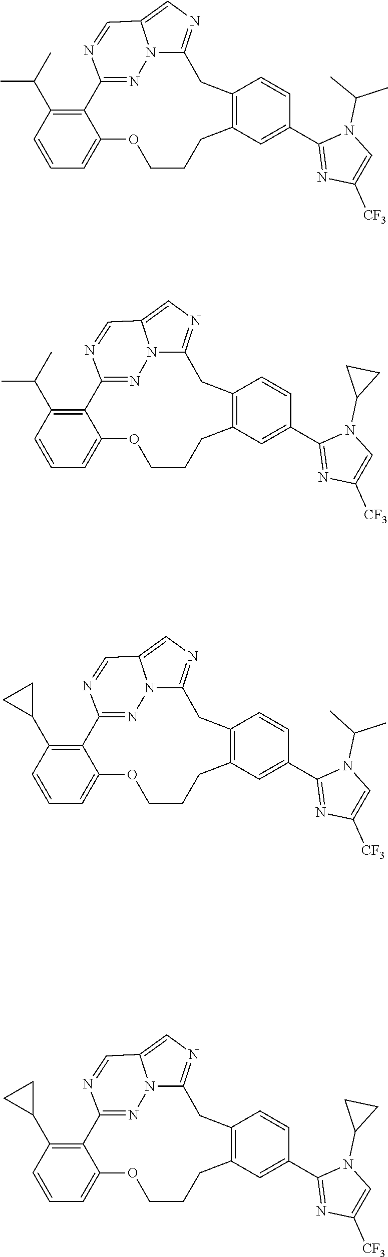

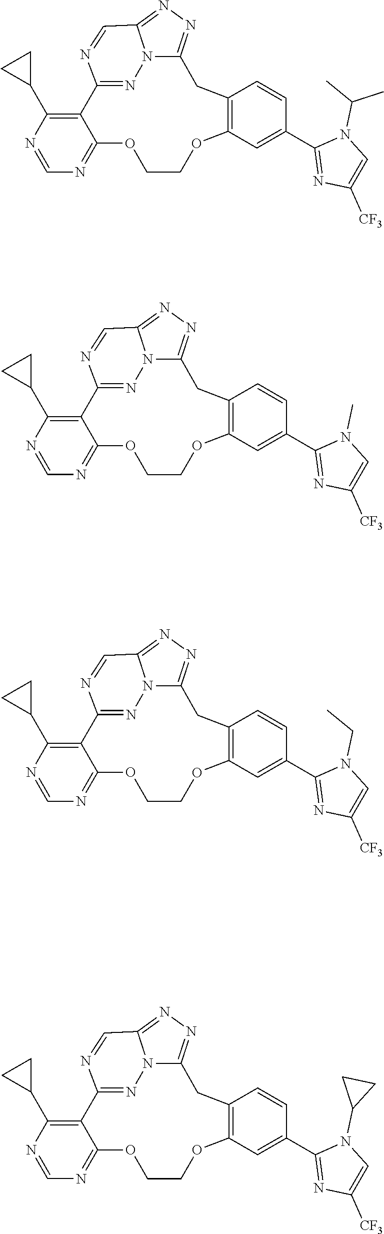 [SMILES: CC(C)n1cc(C(F)(F)F)nc1-c1ccc2c(c1)OCCOc1ncnc(C3CC3)c1-c1ncc3nnc(n3n1)C2 ; Cn1cc(C(F)(F)F)nc1-c1ccc2c(c1)OCCOc1ncnc(C3CC3)c1-c1ncc3nnc(n3n1)C2 ; CCn1cc(C(F)(F)F)nc1-c1ccc2c(c1)OCCOc1ncnc(C3CC3)c1-c1ncc3nnc(n3n1)C2 ; FC(F)(F)c1cn(C2CC2)c(-c2ccc3c(c2)OCCOc2ncnc(C4CC4)c2-c2ncc4nnc(n4n2)C3)n1]

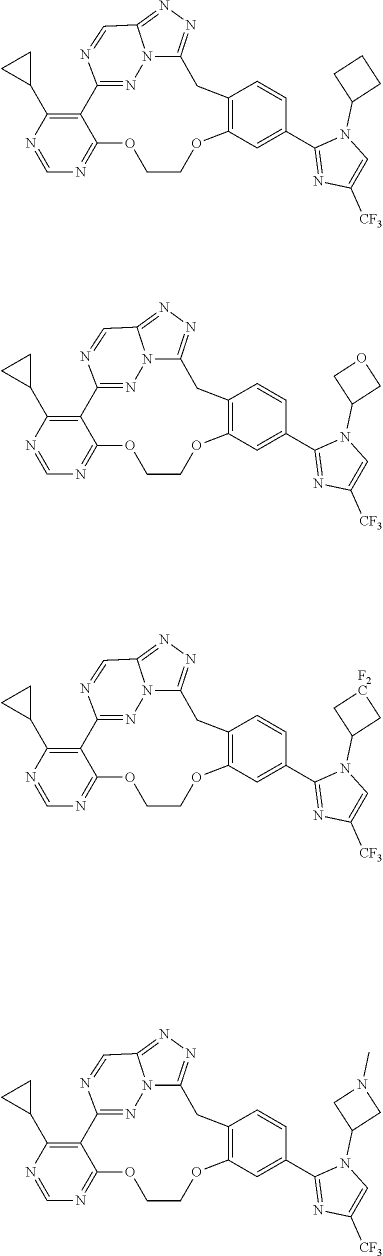 [SMILES: FC(F)(F)c1cn(C2CCC2)c(-c2ccc3c(c2)OCCOc2ncnc(C4CC4)c2-c2ncc4nnc(n4n2)C3)n1 ; FC(F)(F)c1cn(C2COC2)c(-c2ccc3c(c2)OCCOc2ncnc(C4CC4)c2-c2ncc4nnc(n4n2)C3)n1 ; FC1(F)CC(n2cc(C(F)(F)F)nc2-c2ccc3c(c2)OCCOc2ncnc(C4CC4)c2-c2ncc4nnc(n4n2)C3)C1 ; CN1CC(n2cc(C(F)(F)F)nc2-c2ccc3c(c2)OCCOc2ncnc(C4CC4)c2-c2ncc4nnc(n4n2)C3)C1]

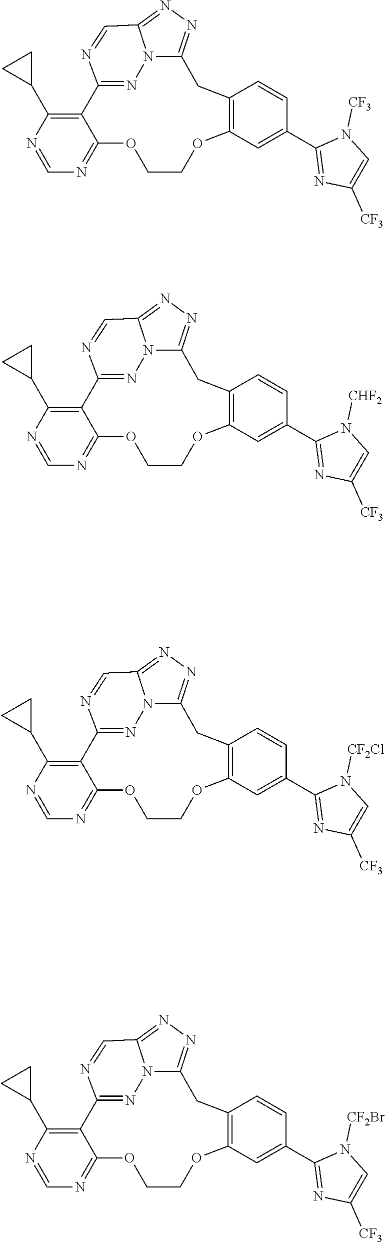

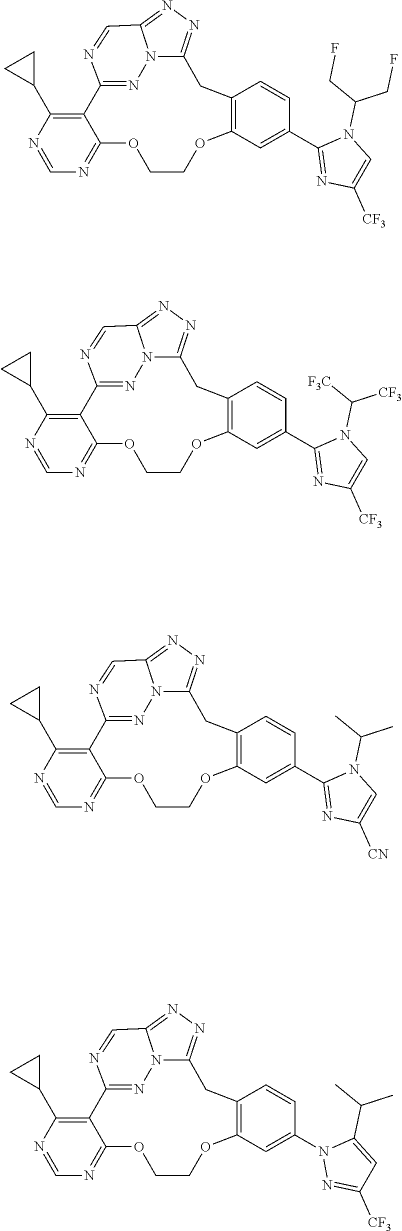 [SMILES: FCC(CF)n1cc(C(F)(F)F)nc1-c1ccc2c(c1)OCCOc1ncnc(C3CC3)c1-c1ncc3nnc(n3n1)C2 ; FC(F)(F)c1cn(C(C(F)(F)F)C(F)(F)F)c(-c2ccc3c(c2)OCCOc2ncnc(C4CC4)c2-c2ncc4nnc(n4n2)C3)n1 ; CC(C)n1cc(C#N)nc1-c1ccc2c(c1)OCCOc1ncnc(C3CC3)c1-c1ncc3nnc(n3n1)C2 ; CC(C)c1cc(C(F)(F)F)nn1-c1ccc2c(c1)OCCOc1ncnc(C3CC3)c1-c1ncc3nnc(n3n1)C2]

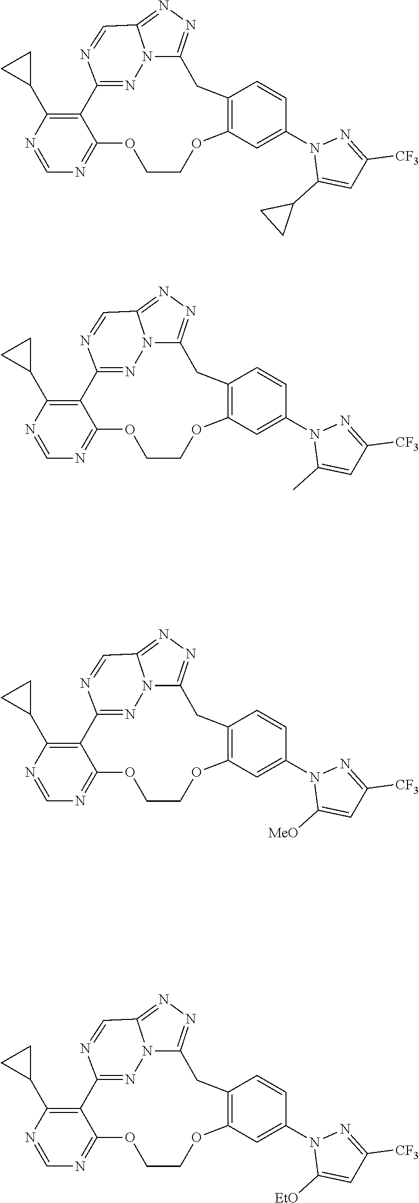 [SMILES: FC(F)(F)c1cc(C2CC2)n(-c2ccc3c(c2)OCCOc2ncnc(C4CC4)c2-c2ncc4nnc(n4n2)C3)n1 ; Cc1cc(C(F)(F)F)nn1-c1ccc2c(c1)OCCOc1ncnc(C3CC3)c1-c1ncc3nnc(n3n1)C2 ; COc1cc(C(F)(F)F)nn1-c1ccc2c(c1)OCCOc1ncnc(C3CC3)c1-c1ncc3nnc(n3n1)C2 ; CCOc1cc(C(F)(F)F)nn1-c1ccc2c(c1)OCCOc1ncnc(C3CC3)c1-c1ncc3nnc(n3n1)C2]

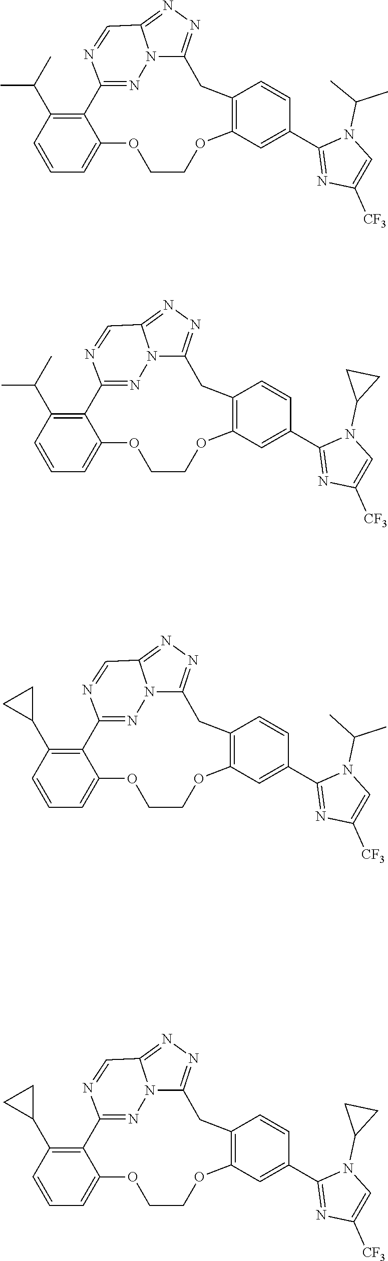

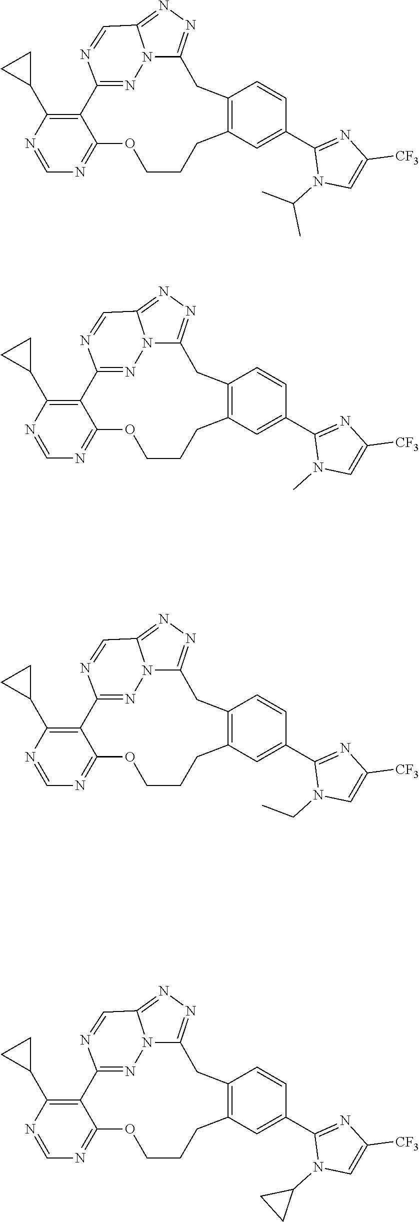 [SMILES: CC(C)n1cc(C(F)(F)F)nc1-c1ccc2c(c1)CCCOc1ncnc(C3CC3)c1-c1ncc3nnc(n3n1)C2 ; Cn1cc(C(F)(F)F)nc1-c1ccc2c(c1)CCCOc1ncnc(C3CC3)c1-c1ncc3nnc(n3n1)C2 ; CCn1cc(C(F)(F)F)nc1-c1ccc2c(c1)CCCOc1ncnc(C3CC3)c1-c1ncc3nnc(n3n1)C2 ; FC(F)(F)c1cn(C2CC2)c(-c2ccc3c(c2)CCCOc2ncnc(C4CC4)c2-c2ncc4nnc(n4n2)C3)n1]

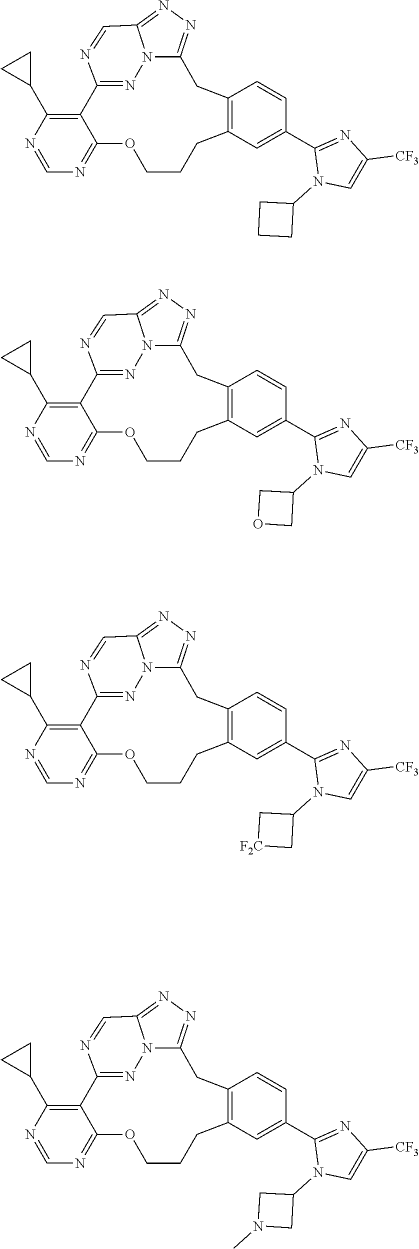 [SMILES: FC(F)(F)c1cn(C2CCC2)c(-c2ccc3c(c2)CCCOc2ncnc(C4CC4)c2-c2ncc4nnc(n4n2)C3)n1 ; FC(F)(F)c1cn(C2COC2)c(-c2ccc3c(c2)CCCOc2ncnc(C4CC4)c2-c2ncc4nnc(n4n2)C3)n1 ; FC1(F)CC(n2cc(C(F)(F)F)nc2-c2ccc3c(c2)CCCOc2ncnc(C4CC4)c2-c2ncc4nnc(n4n2)C3)C1 ; CN1CC(n2cc(C(F)(F)F)nc2-c2ccc3c(c2)CCCOc2ncnc(C4CC4)c2-c2ncc4nnc(n4n2)C3)C1]

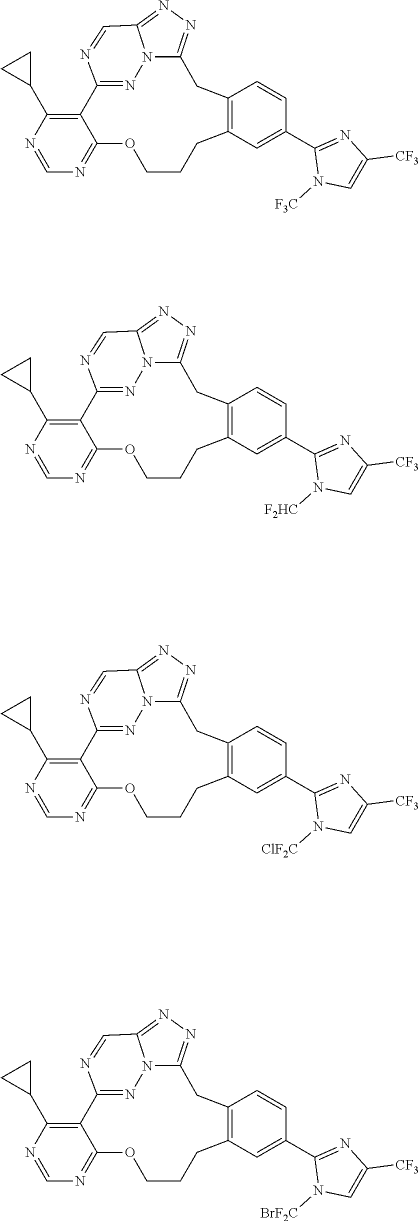

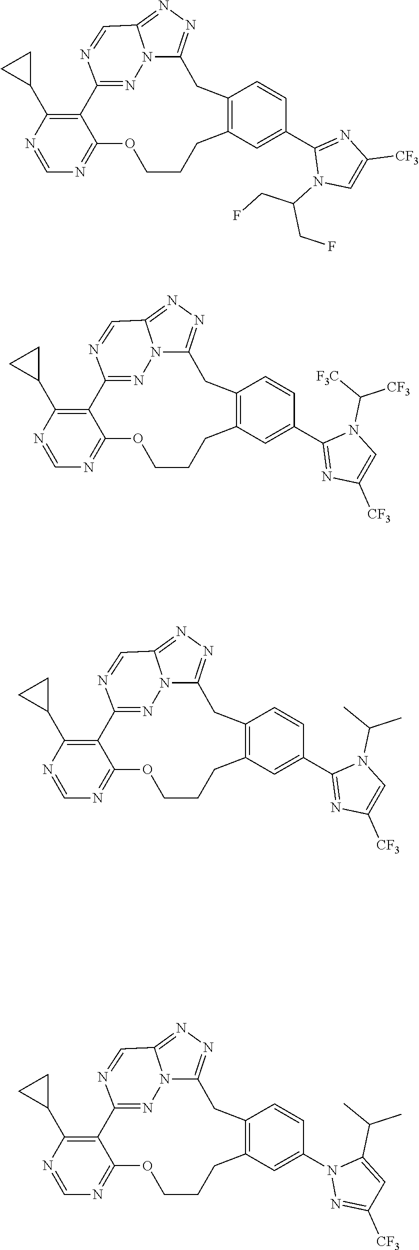 [SMILES: FCC(CF)n1cc(C(F)(F)F)nc1-c1ccc2c(c1)CCCOc1ncnc(C3CC3)c1-c1ncc3nnc(n3n1)C2 ; FC(F)(F)c1cn(C(C(F)(F)F)C(F)(F)F)c(-c2ccc3c(c2)CCCOc2ncnc(C4CC4)c2-c2ncc4nnc(n4n2)C3)n1 ; CC(C)n1cc(C(F)(F)F)nc1-c1ccc2c(c1)CCCOc1ncnc(C3CC3)c1-c1ncc3nnc(n3n1)C2 ; CC(C)c1cc(C(F)(F)F)nn1-c1ccc2c(c1)CCCOc1ncnc(C3CC3)c1-c1ncc3nnc(n3n1)C2]

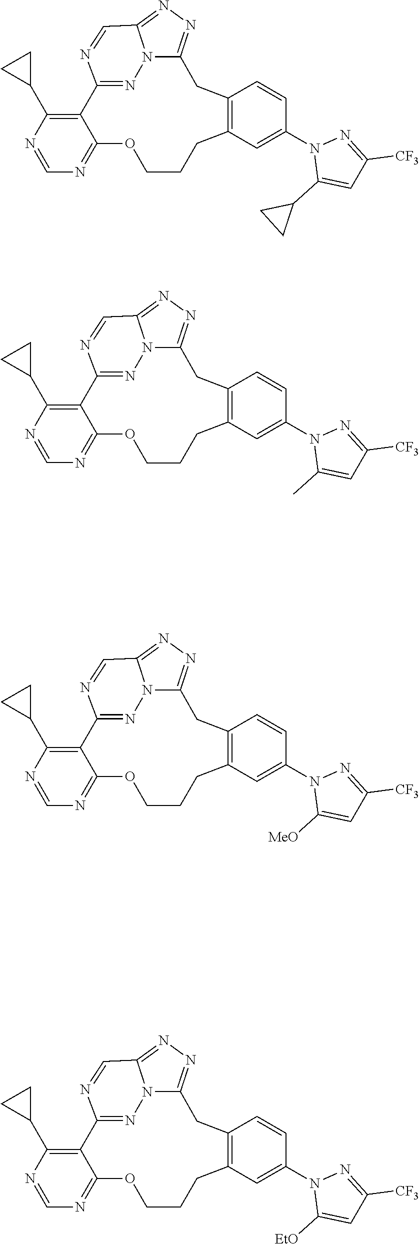 [SMILES: FC(F)(F)c1cc(C2CC2)n(-c2ccc3c(c2)CCCOc2ncnc(C4CC4)c2-c2ncc4nnc(n4n2)C3)n1 ; Cc1cc(C(F)(F)F)nn1-c1ccc2c(c1)CCCOc1ncnc(C3CC3)c1-c1ncc3nnc(n3n1)C2 ; COc1cc(C(F)(F)F)nn1-c1ccc2c(c1)CCCOc1ncnc(C3CC3)c1-c1ncc3nnc(n3n1)C2 ; CCOc1cc(C(F)(F)F)nn1-c1ccc2c(c1)CCCOc1ncnc(C3CC3)c1-c1ncc3nnc(n3n1)C2]

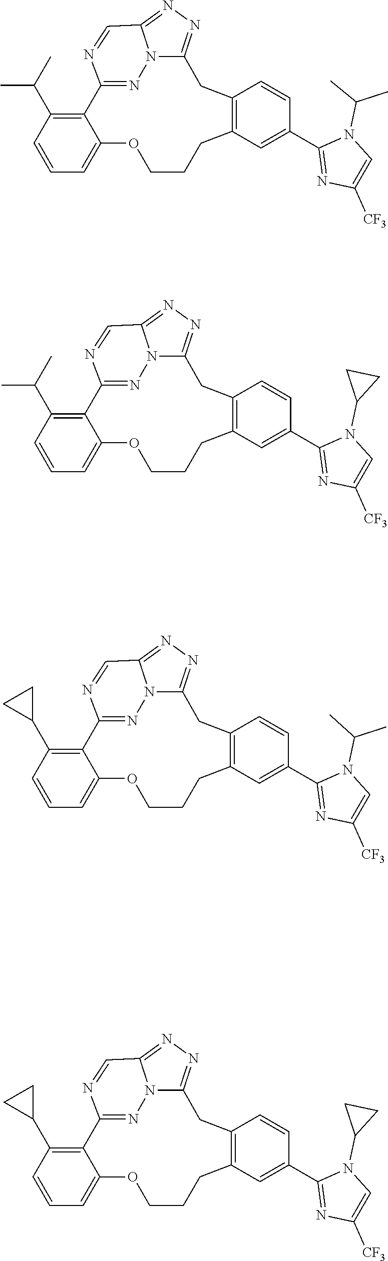

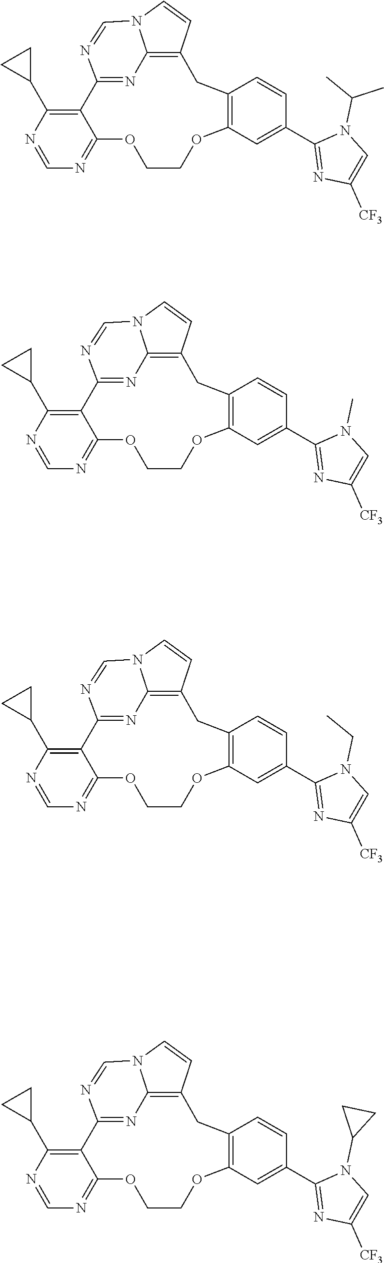 [SMILES: CC(C)n1cc(C(F)(F)F)nc1-c1ccc2c(c1)OCCOc1ncnc(C3CC3)c1-c1ncn3ccc(c3n1)C2 ; Cn1cc(C(F)(F)F)nc1-c1ccc2c(c1)OCCOc1ncnc(C3CC3)c1-c1ncn3ccc(c3n1)C2 ; CCn1cc(C(F)(F)F)nc1-c1ccc2c(c1)OCCOc1ncnc(C3CC3)c1-c1ncn3ccc(c3n1)C2 ; FC(F)(F)c1cn(C2CC2)c(-c2ccc3c(c2)OCCOc2ncnc(C4CC4)c2-c2ncn4ccc(c4n2)C3)n1]

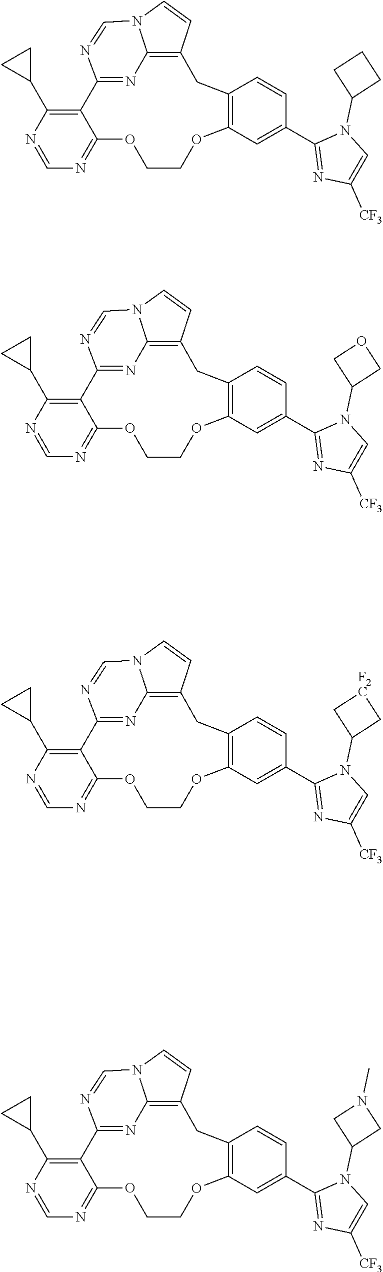 [SMILES: FC(F)(F)c1cn(C2CCC2)c(-c2ccc3c(c2)OCCOc2ncnc(C4CC4)c2-c2ncn4ccc(c4n2)C3)n1 ; FC(F)(F)c1cn(C2COC2)c(-c2ccc3c(c2)OCCOc2ncnc(C4CC4)c2-c2ncn4ccc(c4n2)C3)n1 ; FC1(F)CC(n2cc(C(F)(F)F)nc2-c2ccc3c(c2)OCCOc2ncnc(C4CC4)c2-c2ncn4ccc(c4n2)C3)C1 ; CN1CC(n2cc(C(F)(F)F)nc2-c2ccc3c(c2)OCCOc2ncnc(C4CC4)c2-c2ncn4ccc(c4n2)C3)C1]

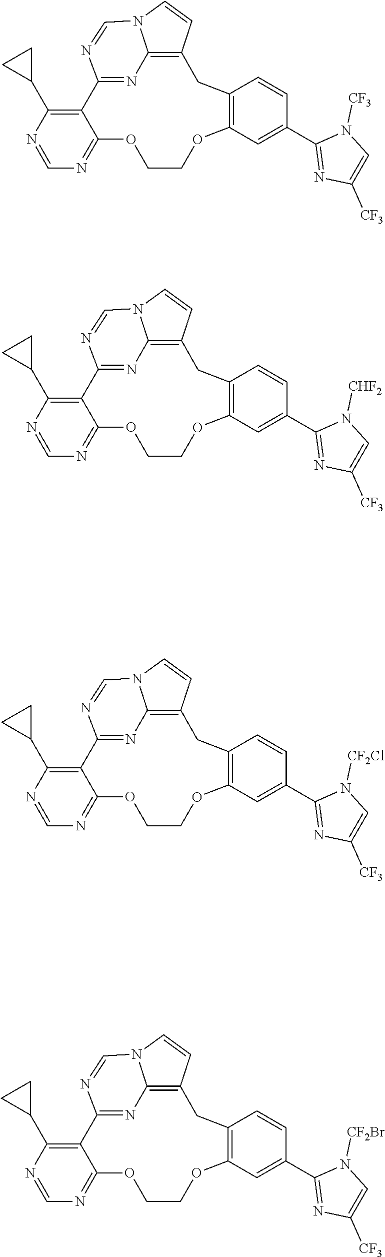

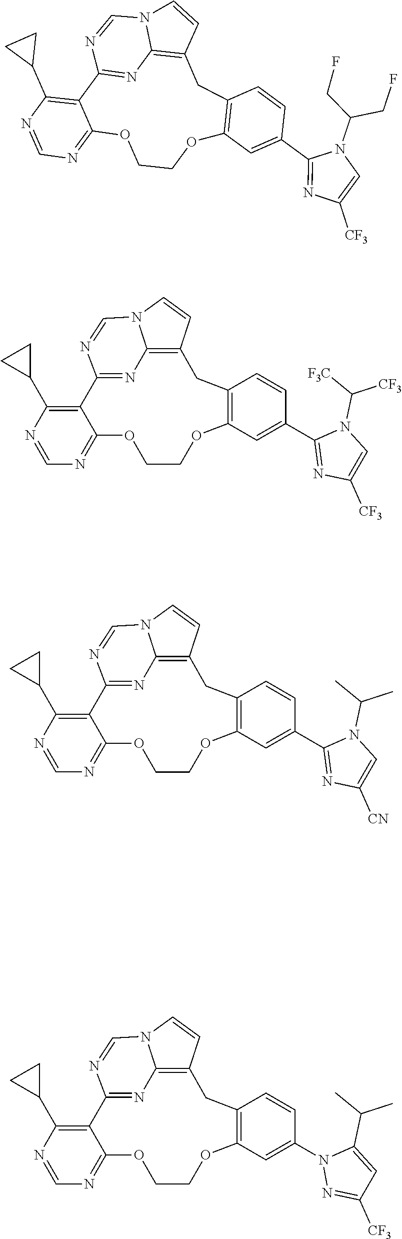 [SMILES: FCC(CF)n1cc(C(F)(F)F)nc1-c1ccc2c(c1)OCCOc1ncnc(C3CC3)c1-c1ncn3ccc(c3n1)C2 ; FC(F)(F)c1cn(C(C(F)(F)F)C(F)(F)F)c(-c2ccc3c(c2)OCCOc2ncnc(C4CC4)c2-c2ncn4ccc(c4n2)C3)n1 ; CC(C)n1cc(C#N)nc1-c1ccc2c(c1)OCCOc1ncnc(C3CC3)c1-c1ncn3ccc(c3n1)C2 ; CC(C)c1cc(C(F)(F)F)nn1-c1ccc2c(c1)OCCOc1ncnc(C3CC3)c1-c1ncn3ccc(c3n1)C2]

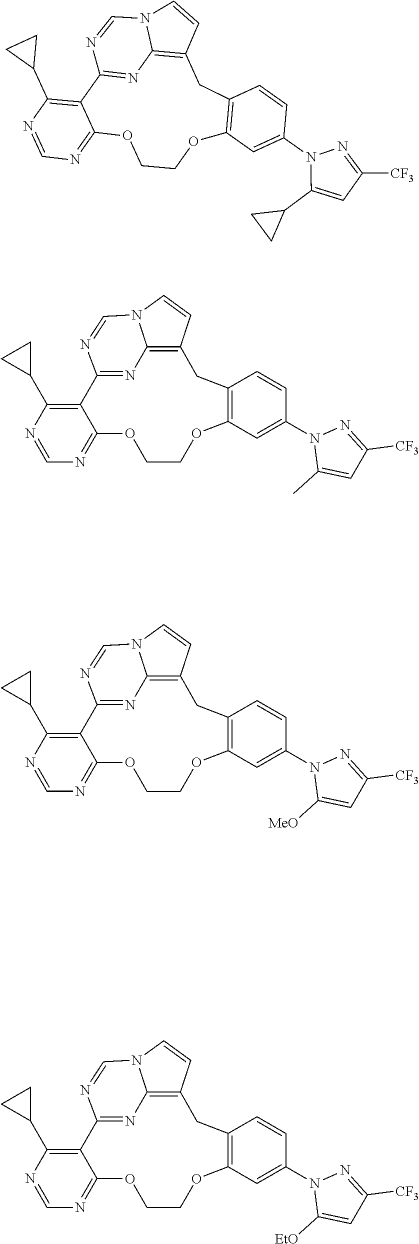 [SMILES: FC(F)(F)c1cc(C2CC2)n(-c2ccc3c(c2)OCCOc2ncnc(C4CC4)c2-c2ncn4ccc(c4n2)C3)n1 ; Cc1cc(C(F)(F)F)nn1-c1ccc2c(c1)OCCOc1ncnc(C3CC3)c1-c1ncn3ccc(c3n1)C2 ; COc1cc(C(F)(F)F)nn1-c1ccc2c(c1)OCCOc1ncnc(C3CC3)c1-c1ncn3ccc(c3n1)C2 ; CCOc1cc(C(F)(F)F)nn1-c1ccc2c(c1)OCCOc1ncnc(C3CC3)c1-c1ncn3ccc(c3n1)C2]

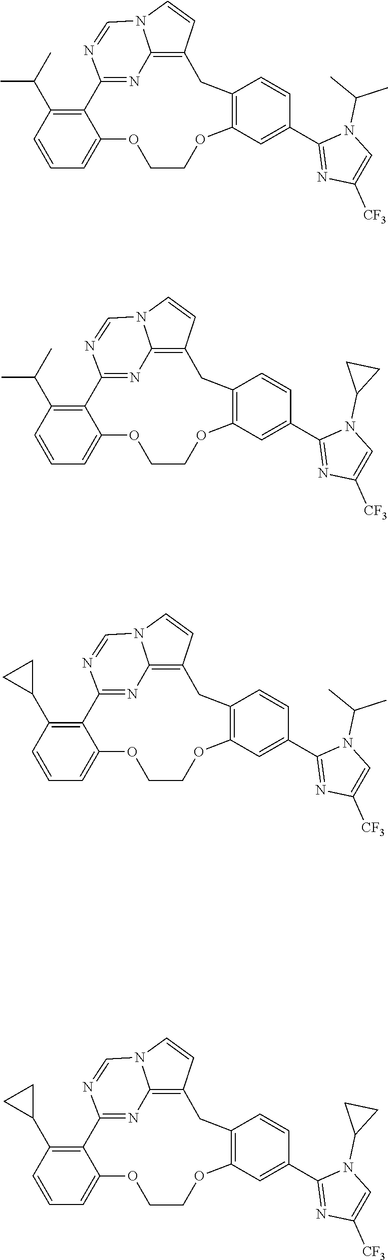

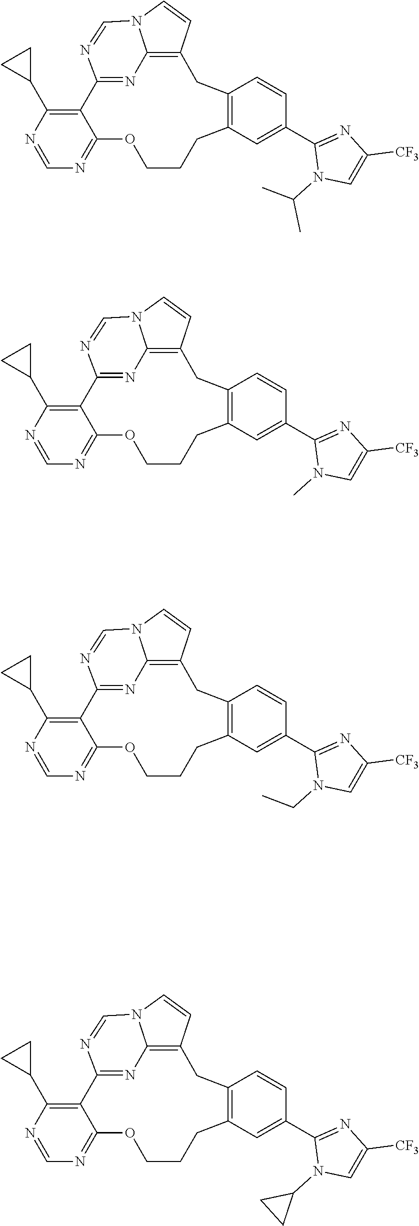 [SMILES: CC(C)n1cc(C(F)(F)F)nc1-c1ccc2c(c1)CCCOc1ncnc(C3CC3)c1-c1ncn3ccc(c3n1)C2 ; Cn1cc(C(F)(F)F)nc1-c1ccc2c(c1)CCCOc1ncnc(C3CC3)c1-c1ncn3ccc(c3n1)C2 ; CCn1cc(C(F)(F)F)nc1-c1ccc2c(c1)CCCOc1ncnc(C3CC3)c1-c1ncn3ccc(c3n1)C2 ; FC(F)(F)c1cn(C2CC2)c(-c2ccc3c(c2)CCCOc2ncnc(C4CC4)c2-c2ncn4ccc(c4n2)C3)n1]

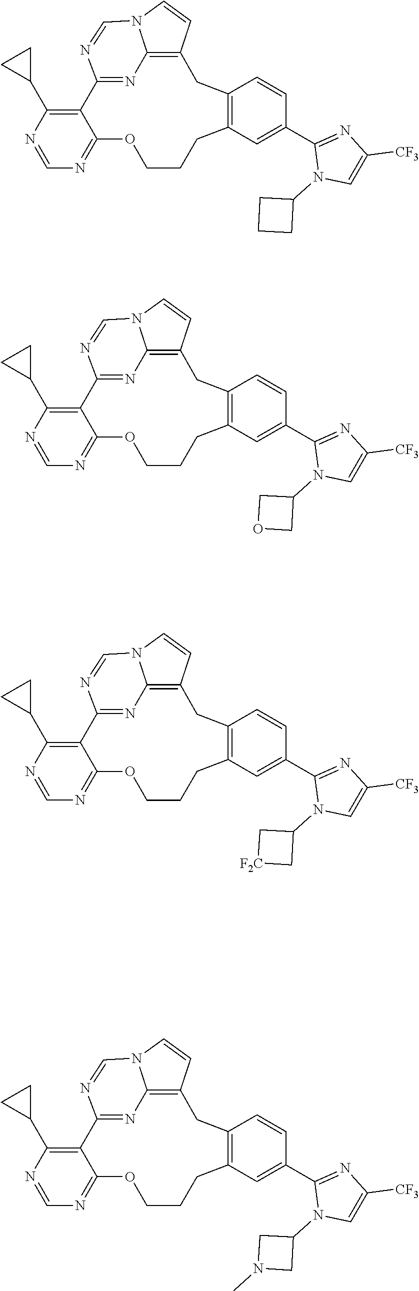 [SMILES: FC(F)(F)c1cn(C2CCC2)c(-c2ccc3c(c2)CCCOc2ncnc(C4CC4)c2-c2ncn4ccc(c4n2)C3)n1 ; FC(F)(F)c1cn(C2COC2)c(-c2ccc3c(c2)CCCOc2ncnc(C4CC4)c2-c2ncn4ccc(c4n2)C3)n1 ; FC1(F)CC(n2cc(C(F)(F)F)nc2-c2ccc3c(c2)CCCOc2ncnc(C4CC4)c2-c2ncn4ccc(c4n2)C3)C1 ; CN1CC(n2cc(C(F)(F)F)nc2-c2ccc3c(c2)CCCOc2ncnc(C4CC4)c2-c2ncn4ccc(c4n2)C3)C1]

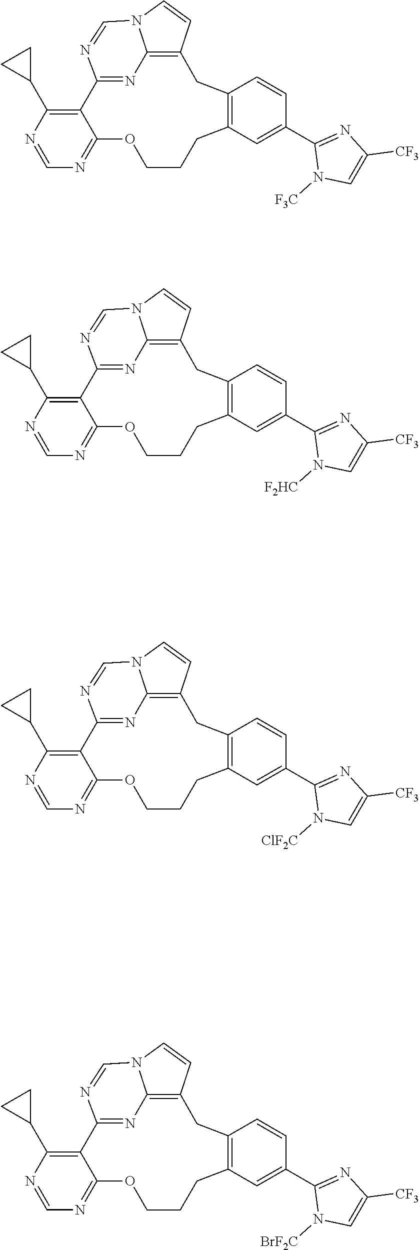

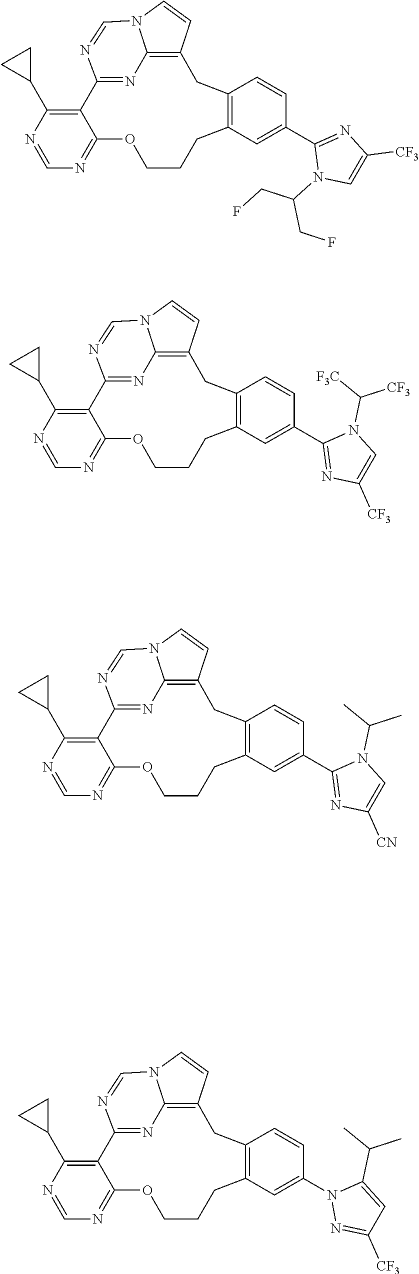 [SMILES: FCC(CF)n1cc(C(F)(F)F)nc1-c1ccc2c(c1)CCCOc1ncnc(C3CC3)c1-c1ncn3ccc(c3n1)C2 ; FC(F)(F)c1cn(C(C(F)(F)F)C(F)(F)F)c(-c2ccc3c(c2)CCCOc2ncnc(C4CC4)c2-c2ncn4ccc(c4n2)C3)n1 ; CC(C)n1cc(C#N)nc1-c1ccc2c(c1)CCCOc1ncnc(C3CC3)c1-c1ncn3ccc(c3n1)C2 ; CC(C)c1cc(C(F)(F)F)nn1-c1ccc2c(c1)CCCOc1ncnc(C3CC3)c1-c1ncn3ccc(c3n1)C2]

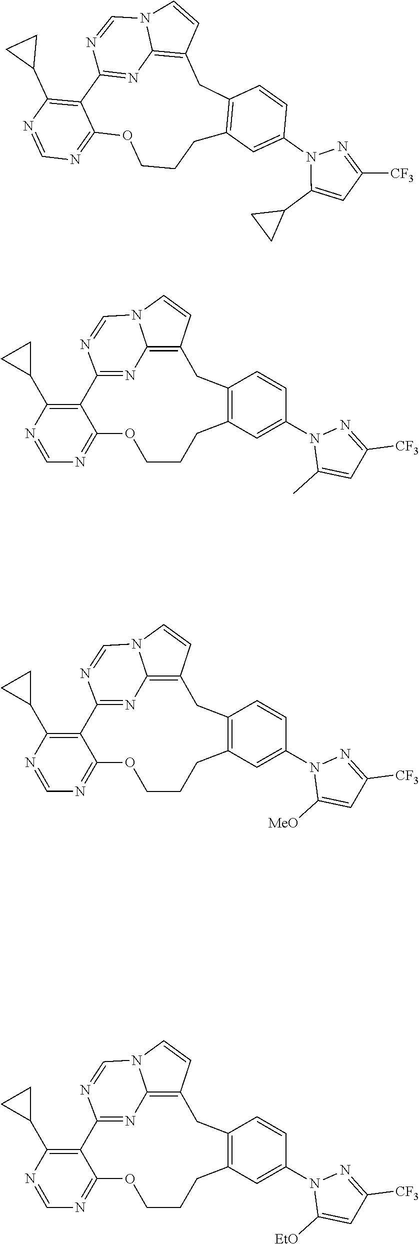 [SMILES: FC(F)(F)c1cc(C2CC2)n(-c2ccc3c(c2)CCCOc2ncnc(C4CC4)c2-c2ncn4ccc(c4n2)C3)n1 ; Cc1cc(C(F)(F)F)nn1-c1ccc2c(c1)CCCOc1ncnc(C3CC3)c1-c1ncn3ccc(c3n1)C2 ; COc1cc(C(F)(F)F)nn1-c1ccc2c(c1)CCCOc1ncnc(C3CC3)c1-c1ncn3ccc(c3n1)C2 ; CCOc1cc(C(F)(F)F)nn1-c1ccc2c(c1)CCCOc1ncnc(C3CC3)c1-c1ncn3ccc(c3n1)C2]

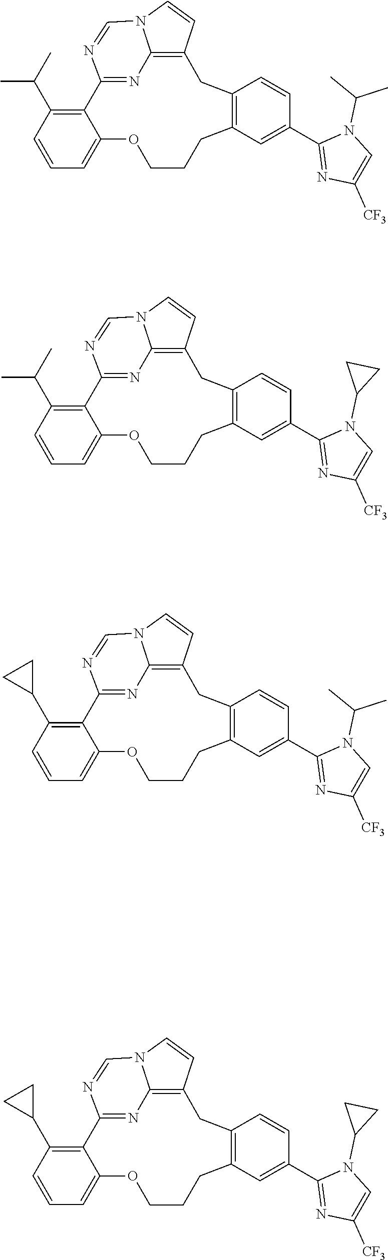

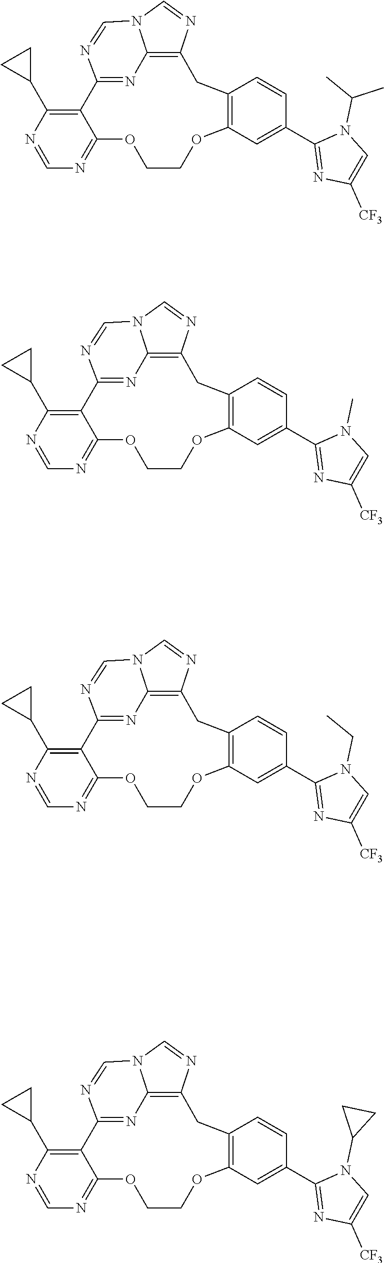 [SMILES: CC(C)n1cc(C(F)(F)F)nc1-c1ccc2c(c1)OCCOc1ncnc(C3CC3)c1-c1ncn3cnc(c3n1)C2 ; Cn1cc(C(F)(F)F)nc1-c1ccc2c(c1)OCCOc1ncnc(C3CC3)c1-c1ncn3cnc(c3n1)C2 ; CCn1cc(C(F)(F)F)nc1-c1ccc2c(c1)OCCOc1ncnc(C3CC3)c1-c1ncn3cnc(c3n1)C2 ; FC(F)(F)c1cn(C2CC2)c(-c2ccc3c(c2)OCCOc2ncnc(C4CC4)c2-c2ncn4cnc(c4n2)C3)n1]

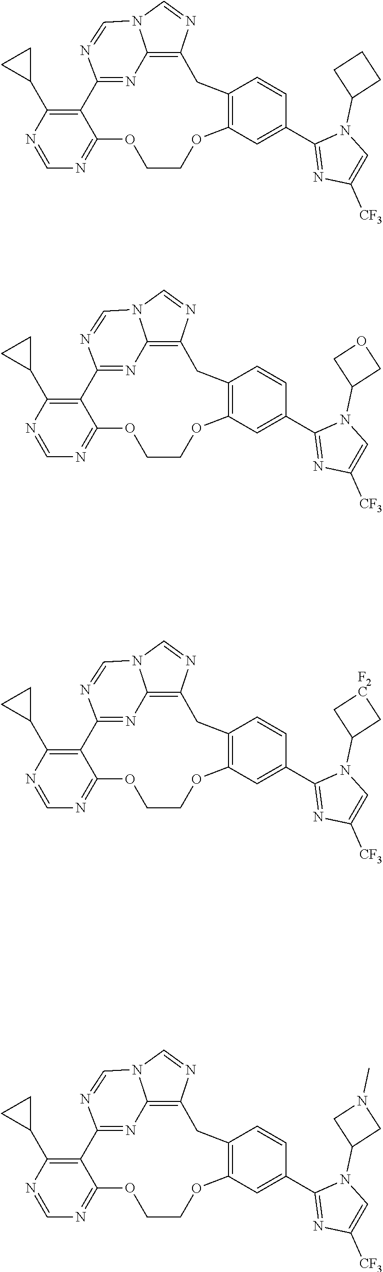 [SMILES: FC(F)(F)c1cn(C2CCC2)c(-c2ccc3c(c2)OCCOc2ncnc(C4CC4)c2-c2ncn4cnc(c4n2)C3)n1 ; FC(F)(F)c1cn(C2COC2)c(-c2ccc3c(c2)OCCOc2ncnc(C4CC4)c2-c2ncn4cnc(c4n2)C3)n1 ; FC1(F)CC(n2cc(C(F)(F)F)nc2-c2ccc3c(c2)OCCOc2ncnc(C4CC4)c2-c2ncn4cnc(c4n2)C3)C1 ; CN1CC(n2cc(C(F)(F)F)nc2-c2ccc3c(c2)OCCOc2ncnc(C4CC4)c2-c2ncn4cnc(c4n2)C3)C1]

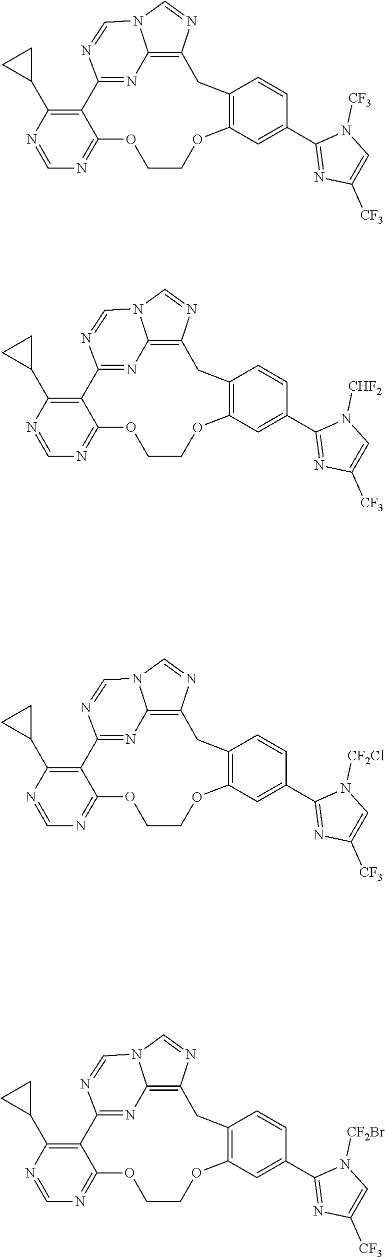

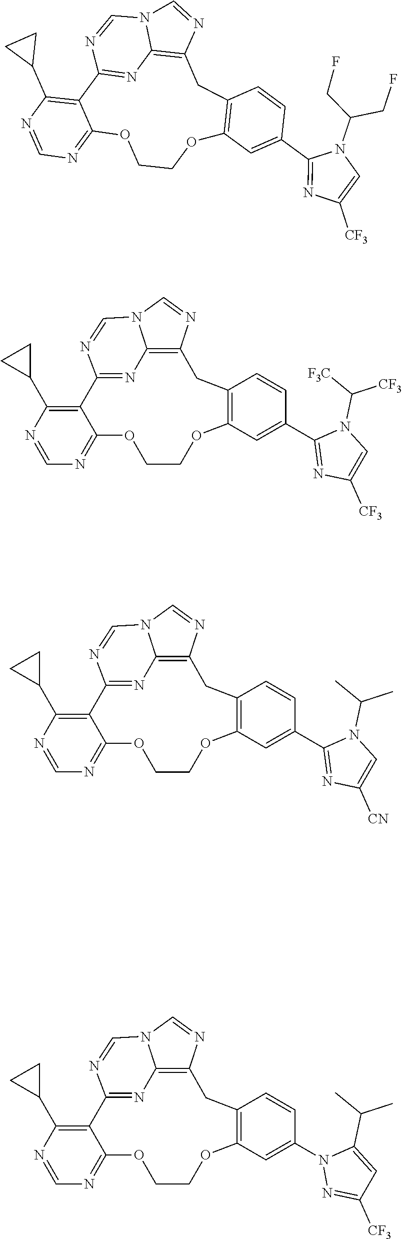 [SMILES: FCC(CF)n1cc(C(F)(F)F)nc1-c1ccc2c(c1)OCCOc1ncnc(C3CC3)c1-c1ncn3cnc(c3n1)C2 ; FC(F)(F)c1cn(C(C(F)(F)F)C(F)(F)F)c(-c2ccc3c(c2)OCCOc2ncnc(C4CC4)c2-c2ncn4cnc(c4n2)C3)n1 ; CC(C)n1cc(C#N)nc1-c1ccc2c(c1)OCCOc1ncnc(C3CC3)c1-c1ncn3cnc(c3n1)C2 ; CC(C)c1cc(C(F)(F)F)nn1-c1ccc2c(c1)OCCOc1ncnc(C3CC3)c1-c1ncn3cnc(c3n1)C2]

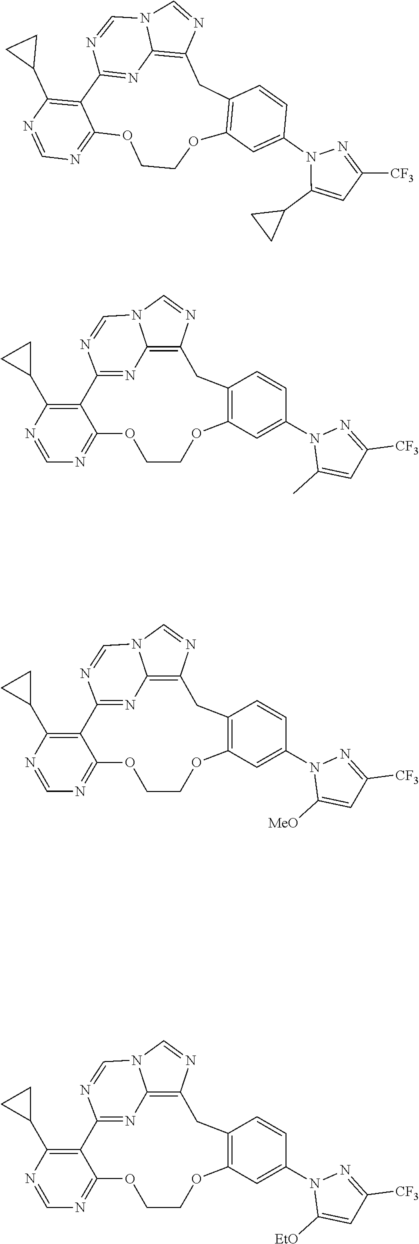 [SMILES: FC(F)(F)c1cc(C2CC2)n(-c2ccc3c(c2)OCCOc2ncnc(C4CC4)c2-c2ncn4cnc(c4n2)C3)n1 ; Cc1cc(C(F)(F)F)nn1-c1ccc2c(c1)OCCOc1ncnc(C3CC3)c1-c1ncn3cnc(c3n1)C2 ; COc1cc(C(F)(F)F)nn1-c1ccc2c(c1)OCCOc1ncnc(C3CC3)c1-c1ncn3cnc(c3n1)C2 ; CCOc1cc(C(F)(F)F)nn1-c1ccc2c(c1)OCCOc1ncnc(C3CC3)c1-c1ncn3cnc(c3n1)C2]

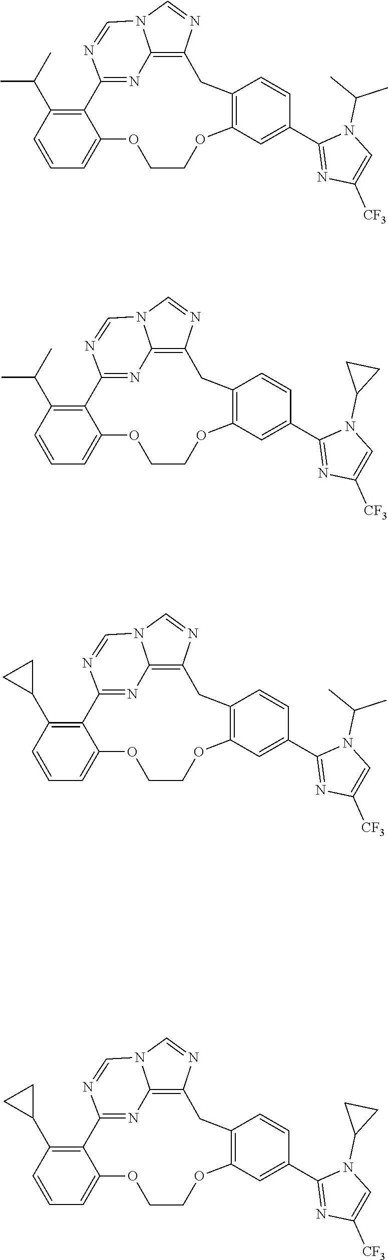

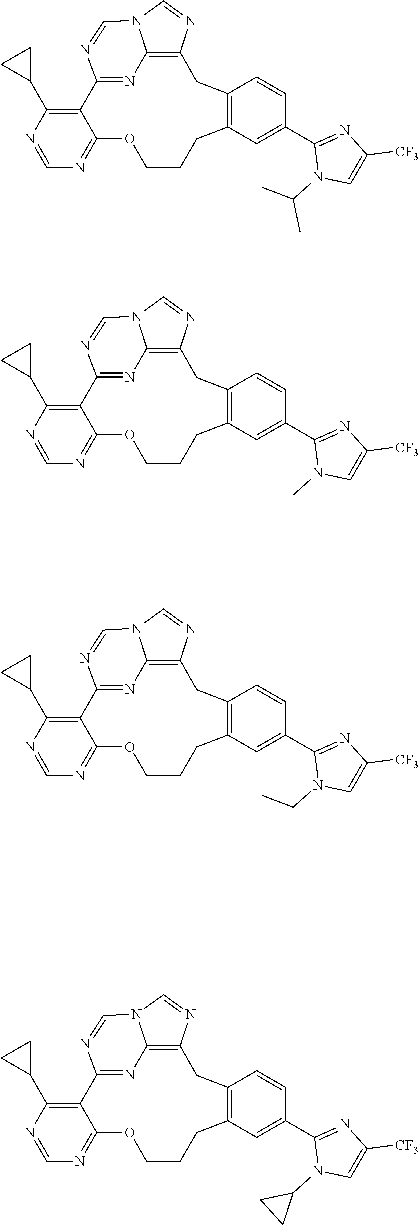 [SMILES: CC(C)n1cc(C(F)(F)F)nc1-c1ccc2c(c1)CCCOc1ncnc(C3CC3)c1-c1ncn3cnc(c3n1)C2 ; Cn1cc(C(F)(F)F)nc1-c1ccc2c(c1)CCCOc1ncnc(C3CC3)c1-c1ncn3cnc(c3n1)C2 ; CCn1cc(C(F)(F)F)nc1-c1ccc2c(c1)CCCOc1ncnc(C3CC3)c1-c1ncn3cnc(c3n1)C2 ; FC(F)(F)c1cn(C2CC2)c(-c2ccc3c(c2)CCCOc2ncnc(C4CC4)c2-c2ncn4cnc(c4n2)C3)n1]

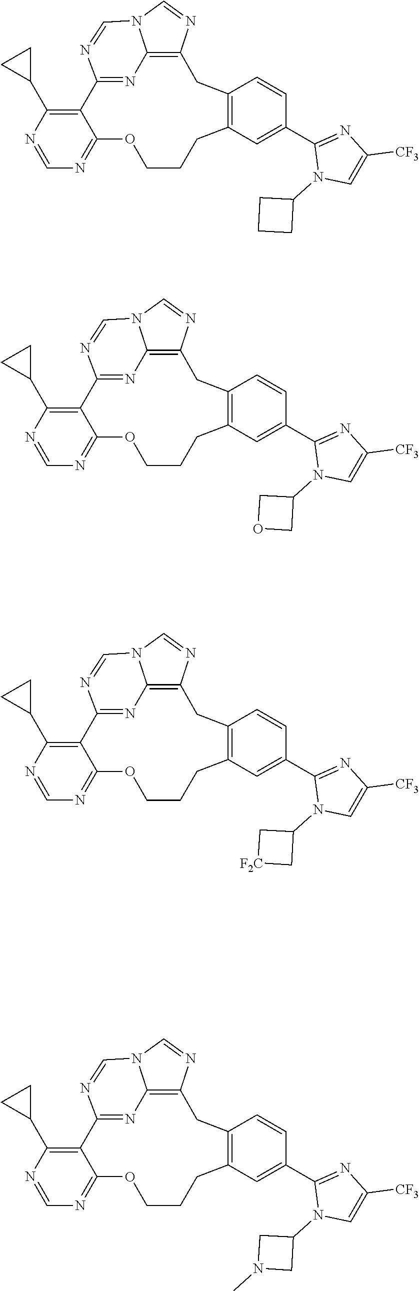 [SMILES: FC(F)(F)c1cn(C2CCC2)c(-c2ccc3c(c2)CCCOc2ncnc(C4CC4)c2-c2ncn4cnc(c4n2)C3)n1 ; FC(F)(F)c1cn(C2COC2)c(-c2ccc3c(c2)CCCOc2ncnc(C4CC4)c2-c2ncn4cnc(c4n2)C3)n1 ; FC1(F)CC(n2cc(C(F)(F)F)nc2-c2ccc3c(c2)CCCOc2ncnc(C4CC4)c2-c2ncn4cnc(c4n2)C3)C1 ; CN1CC(n2cc(C(F)(F)F)nc2-c2ccc3c(c2)CCCOc2ncnc(C4CC4)c2-c2ncn4cnc(c4n2)C3)C1]

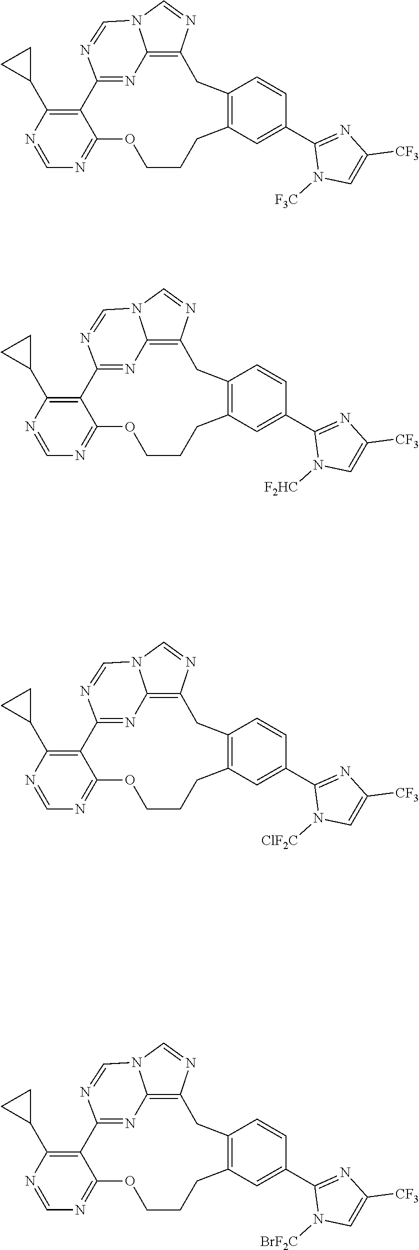

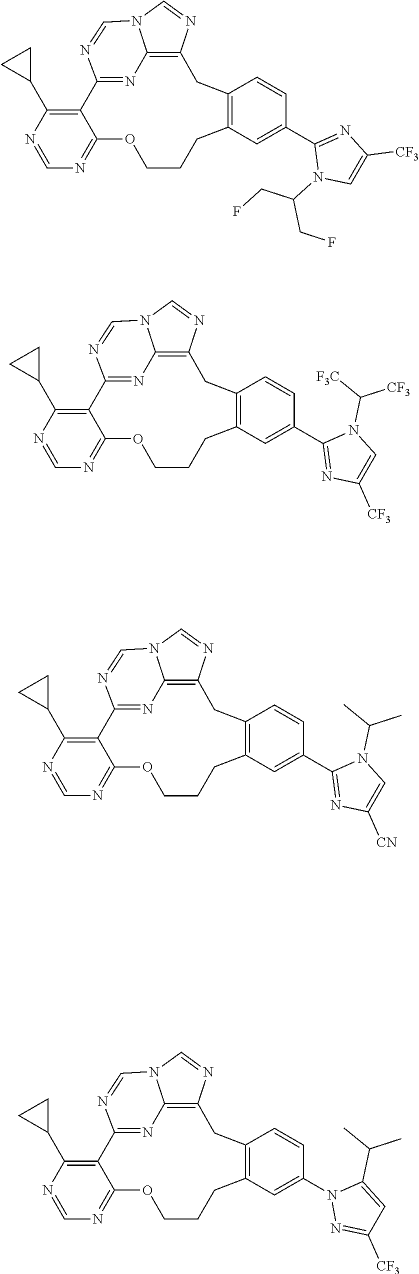 [SMILES: FCC(CF)n1cc(C(F)(F)F)nc1-c1ccc2c(c1)CCCOc1ncnc(C3CC3)c1-c1ncn3cnc(c3n1)C2 ; FC(F)(F)c1cn(C(C(F)(F)F)C(F)(F)F)c(-c2ccc3c(c2)CCCOc2ncnc(C4CC4)c2-c2ncn4cnc(c4n2)C3)n1 ; CC(C)n1cc(C#N)nc1-c1ccc2c(c1)CCCOc1ncnc(C3CC3)c1-c1ncn3cnc(c3n1)C2 ; CC(C)c1cc(C(F)(F)F)nn1-c1ccc2c(c1)CCCOc1ncnc(C3CC3)c1-c1ncn3cnc(c3n1)C2]

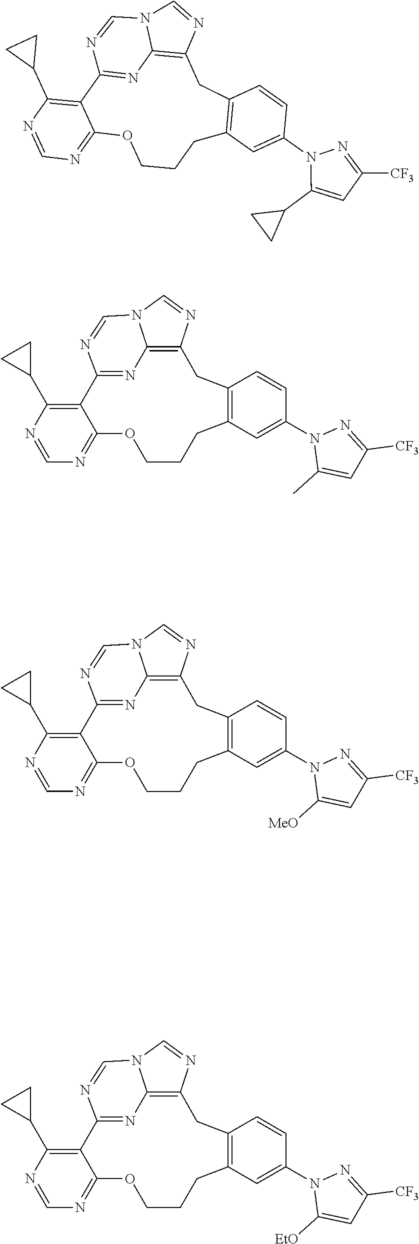 [SMILES: FC(F)(F)c1cc(C2CC2)n(-c2ccc3c(c2)CCCOc2ncnc(C4CC4)c2-c2ncn4cnc(c4n2)C3)n1 ; Cc1cc(C(F)(F)F)nn1-c1ccc2c(c1)CCCOc1ncnc(C3CC3)c1-c1ncn3cnc(c3n1)C2 ; COc1cc(C(F)(F)F)nn1-c1ccc2c(c1)CCCOc1ncnc(C3CC3)c1-c1ncn3cnc(c3n1)C2 ; CCOc1cc(C(F)(F)F)nn1-c1ccc2c(c1)CCCOc1ncnc(C3CC3)c1-c1ncn3cnc(c3n1)C2]

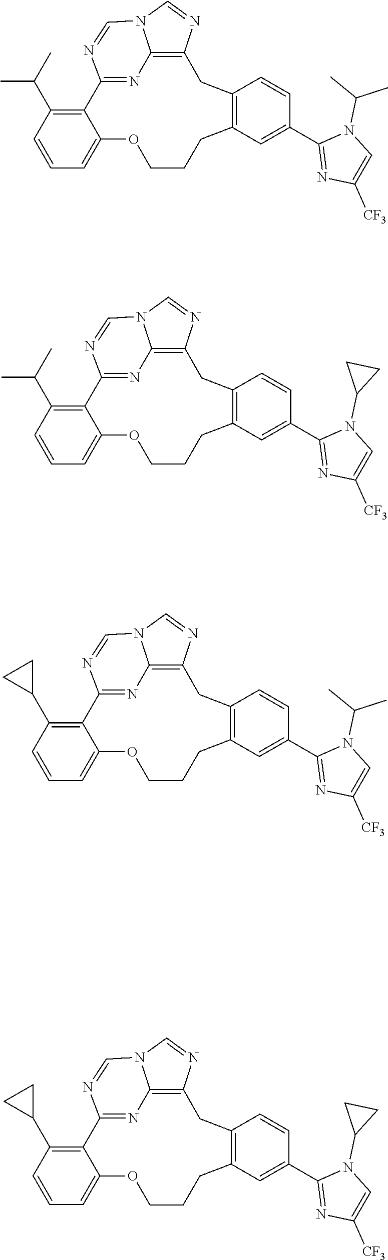

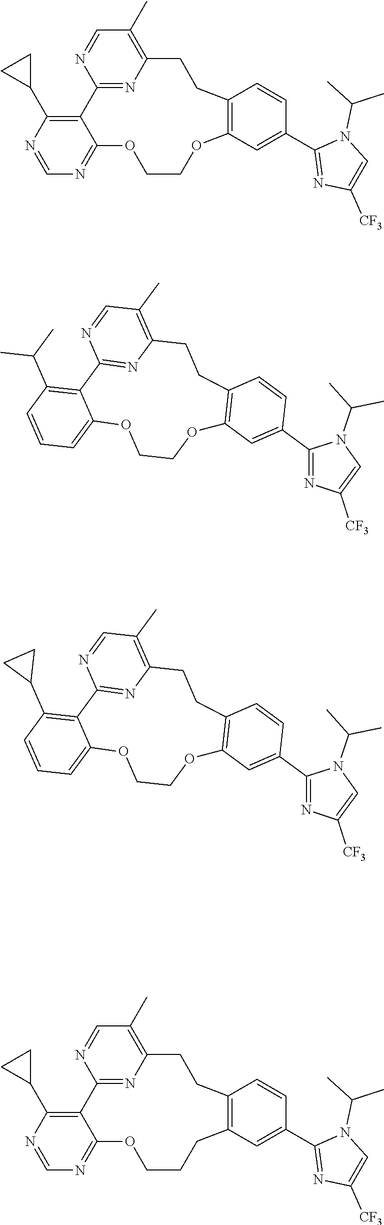

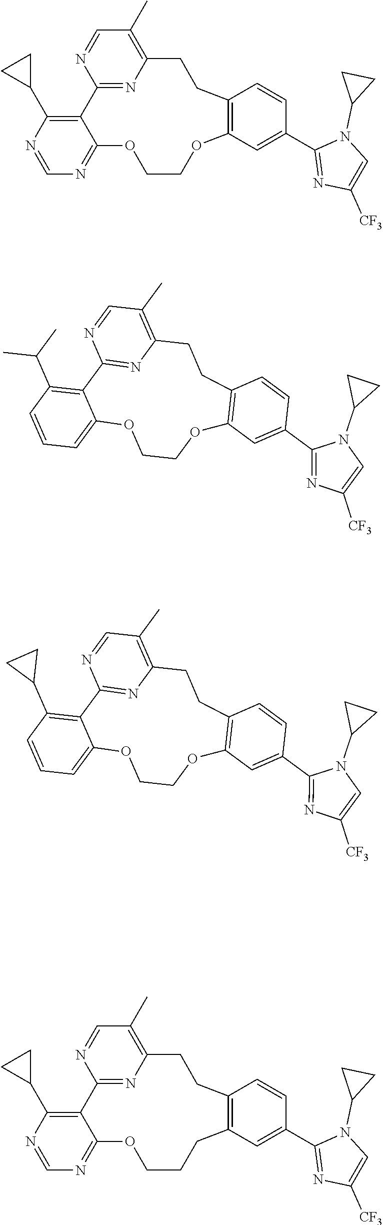 [SMILES: Cc1cnc2nc1CCc1ccc(-c3nc(C(F)(F)F)cn3C3CC3)cc1OCCOc1ncnc(C3CC3)c1-2 ; Cc1cnc2nc1CCc1ccc(-c3nc(C(F)(F)F)cn3C3CC3)cc1OCCOc1cccc(C(C)C)c1-2 ; Cc1cnc2nc1CCc1ccc(-c3nc(C(F)(F)F)cn3C3CC3)cc1OCCOc1cccc(C3CC3)c1-2 ; Cc1cnc2nc1CCc1ccc(-c3nc(C(F)(F)F)cn3C3CC3)cc1CCCOc1ncnc(C3CC3)c1-2]

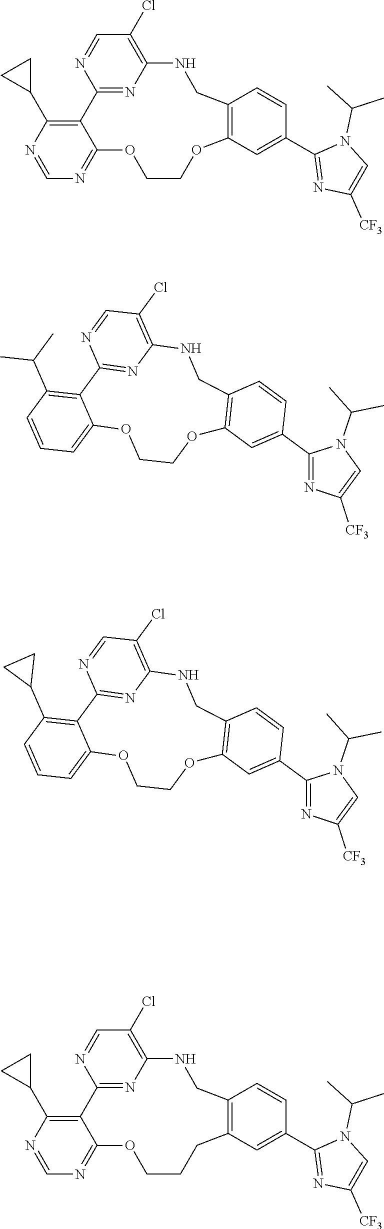

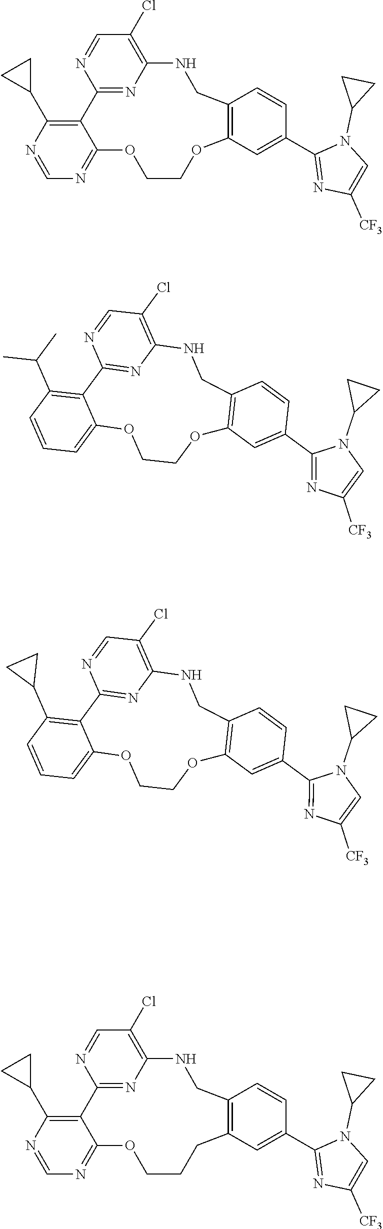 [SMILES: FC(F)(F)c1cn(C2CC2)c(-c2ccc3c(c2)OCCOc2ncnc(C4CC4)c2-c2ncc(Cl)c(n2)NC3)n1 ; CC(C)c1cccc2c1-c1ncc(Cl)c(n1)NCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncc(Cl)c(n2)NC3)n1 ; FC(F)(F)c1cn(C2CC2)c(-c2ccc3c(c2)CCCOc2ncnc(C4CC4)c2-c2ncc(Cl)c(n2)NC3)n1]

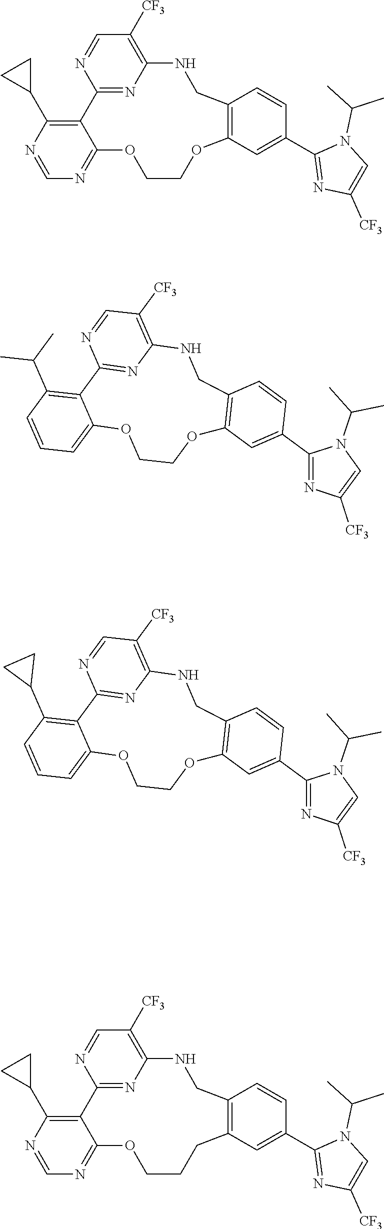

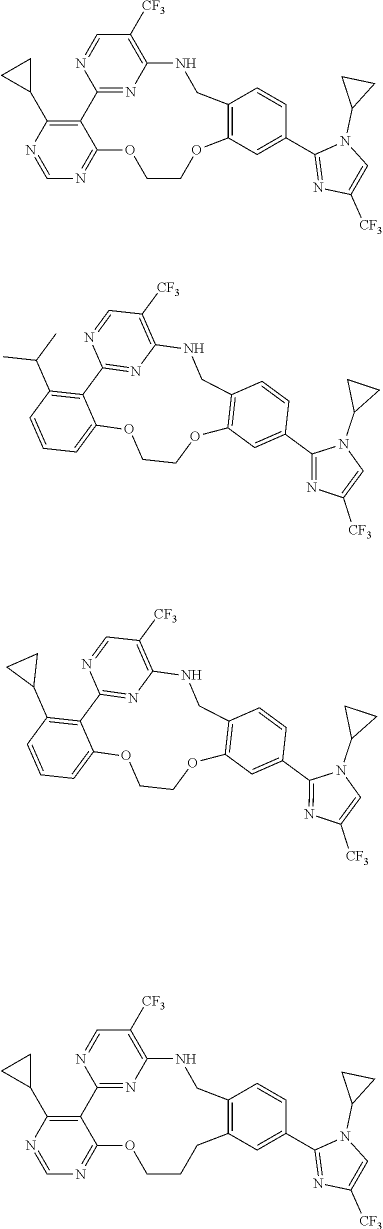 [SMILES: FC(F)(F)c1cn(C2CC2)c(-c2ccc3c(c2)OCCOc2ncnc(C4CC4)c2-c2ncc(C(F)(F)F)c(n2)NC3)n1 ; CC(C)c1cccc2c1-c1ncc(C(F)(F)F)c(n1)NCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncc(C(F)(F)F)c(n2)NC3)n1 ; FC(F)(F)c1cn(C2CC2)c(-c2ccc3c(c2)CCCOc2ncnc(C4CC4)c2-c2ncc(C(F)(F)F)c(n2)NC3)n1]

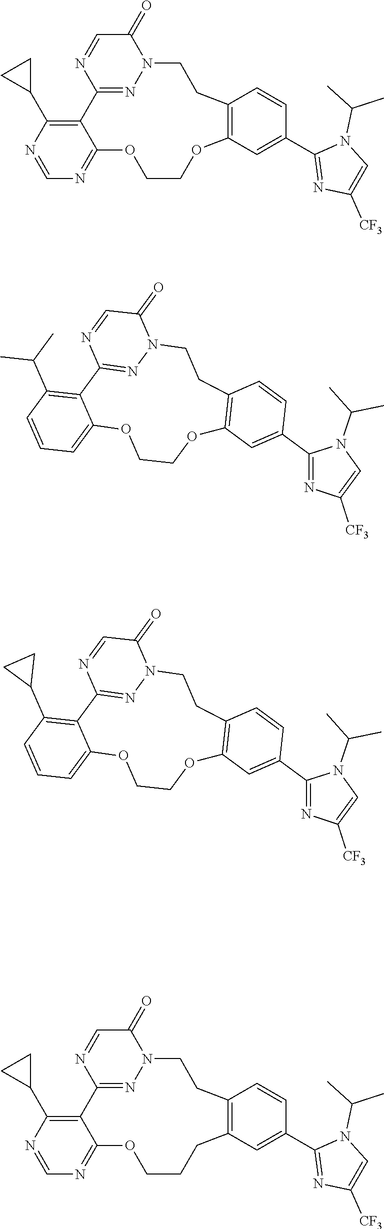

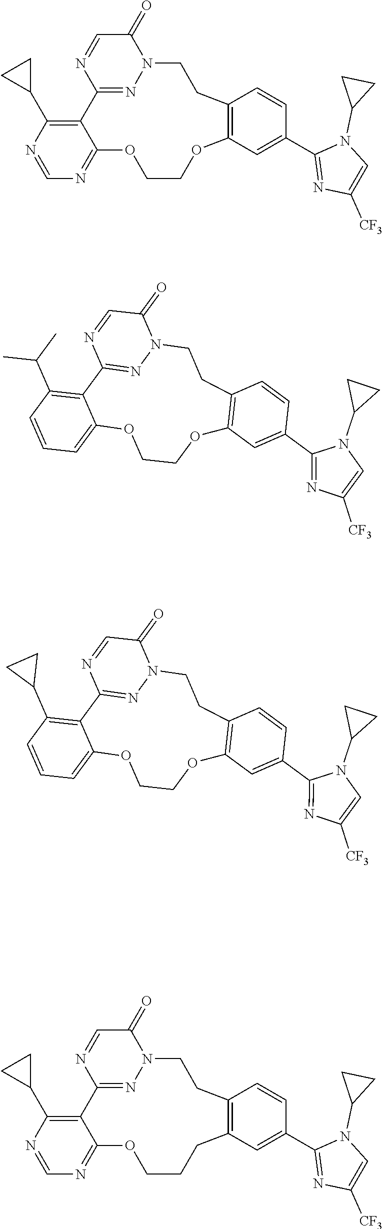 [SMILES: O=c1cnc2nn1CCc1ccc(-c3nc(C(F)(F)F)cn3C3CC3)cc1OCCOc1ncnc(C3CC3)c1-2 ; CC(C)c1cccc2c1-c1ncc(=O)n(n1)CCc1ccc(-c3nc(C(F)(F)F)cn3C3CC3)cc1OCCO2 ; O=c1cnc2nn1CCc1ccc(-c3nc(C(F)(F)F)cn3C3CC3)cc1OCCOc1cccc(C3CC3)c1-2 ; O=c1cnc2nn1CCc1ccc(-c3nc(C(F)(F)F)cn3C3CC3)cc1CCCOc1ncnc(C3CC3)c1-2]

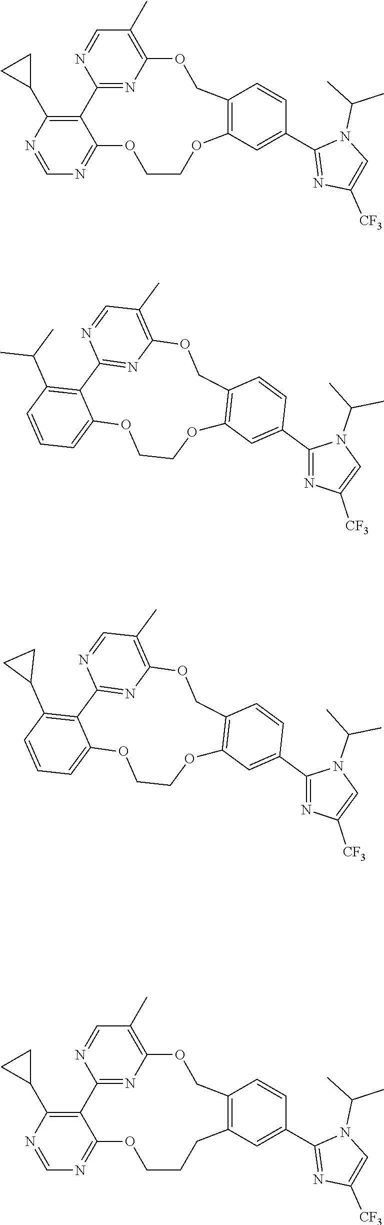

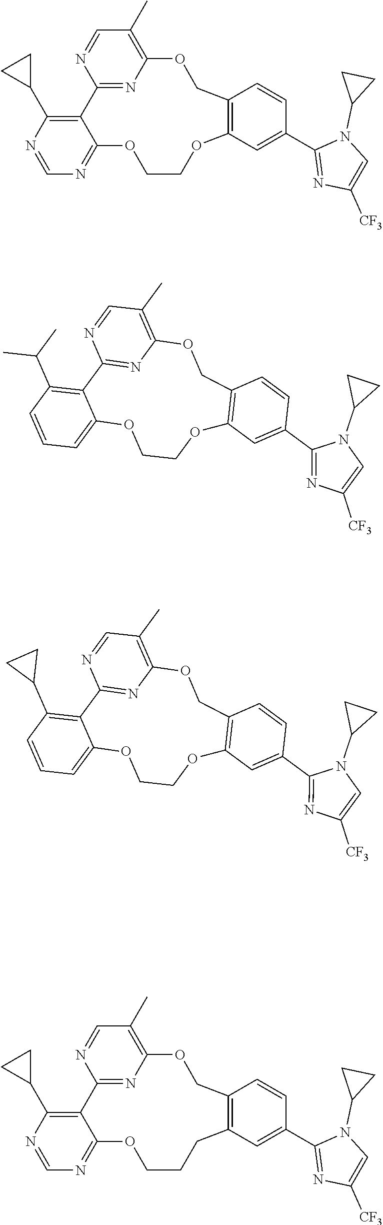 [SMILES: Cc1cnc2nc1OCc1ccc(-c3nc(C(F)(F)F)cn3C3CC3)cc1OCCOc1ncnc(C3CC3)c1-2 ; Cc1cnc2nc1OCc1ccc(-c3nc(C(F)(F)F)cn3C3CC3)cc1OCCOc1cccc(C(C)C)c1-2 ; Cc1cnc2nc1OCc1ccc(-c3nc(C(F)(F)F)cn3C3CC3)cc1OCCOc1cccc(C3CC3)c1-2 ; Cc1cnc2nc1OCc1ccc(-c3nc(C(F)(F)F)cn3C3CC3)cc1CCCOc1ncnc(C3CC3)c1-2]

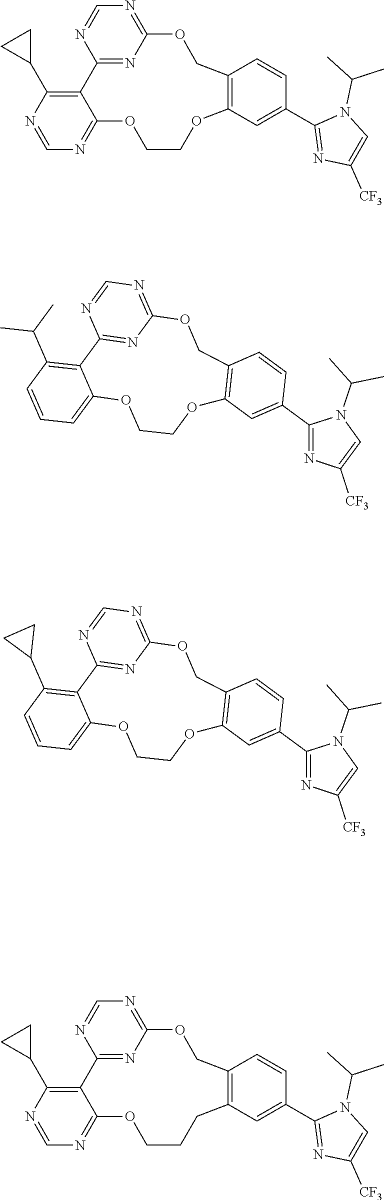

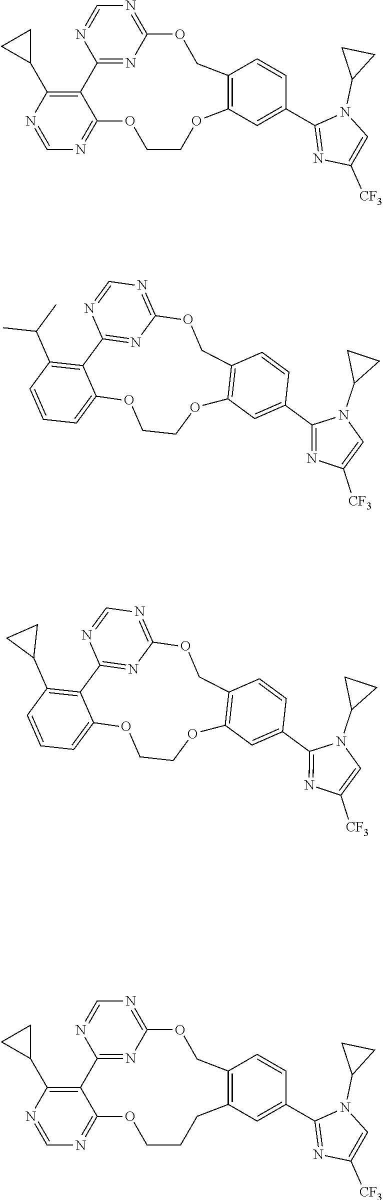 [SMILES: FC(F)(F)c1cn(C2CC2)c(-c2ccc3c(c2)OCCOc2ncnc(C4CC4)c2-c2ncnc(n2)OC3)n1 ; CC(C)c1cccc2c1-c1ncnc(n1)OCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncnc(n2)OC3)n1 ; FC(F)(F)c1cn(C2CC2)c(-c2ccc3c(c2)CCCOc2ncnc(C4CC4)c2-c2ncnc(n2)OC3)n1]

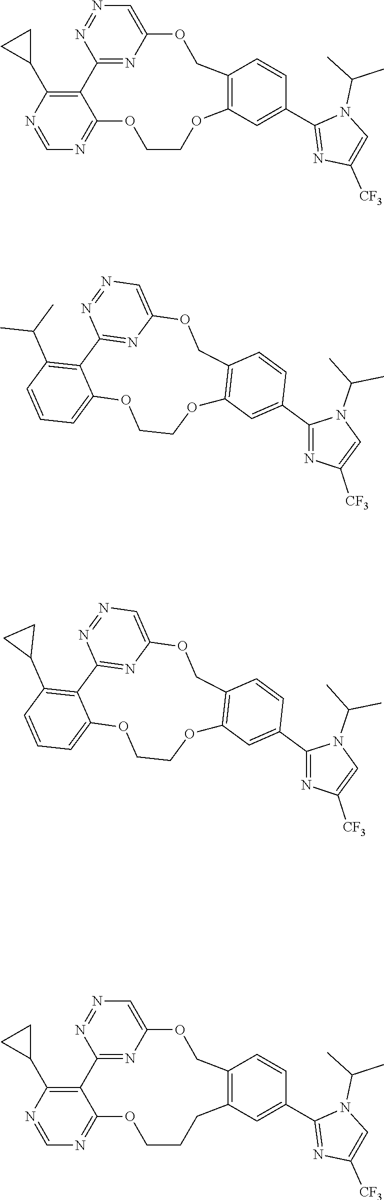

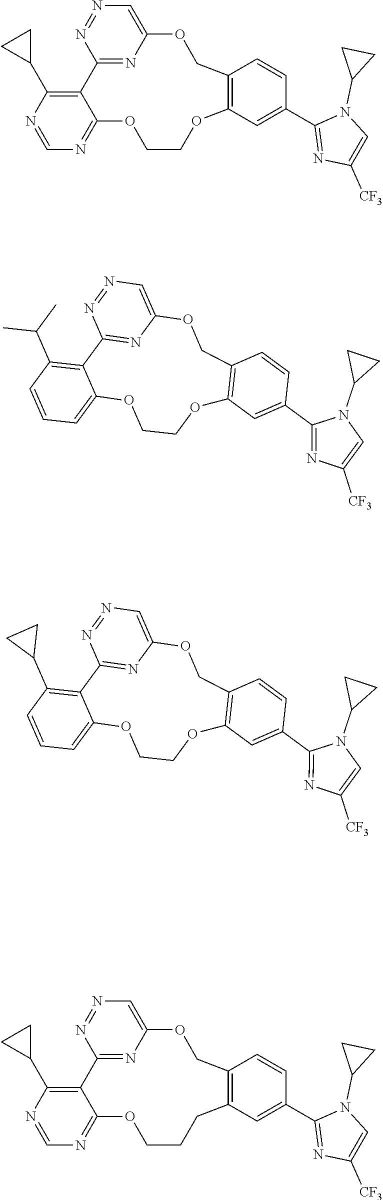 [SMILES: FC(F)(F)c1cn(C2CC2)c(-c2ccc3c(c2)OCCOc2ncnc(C4CC4)c2-c2nncc(n2)OC3)n1 ; CC(C)c1cccc2c1-c1nncc(n1)OCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2nncc(n2)OC3)n1 ; FC(F)(F)c1cn(C2CC2)c(-c2ccc3c(c2)CCCOc2ncnc(C4CC4)c2-c2nncc(n2)OC3)n1]

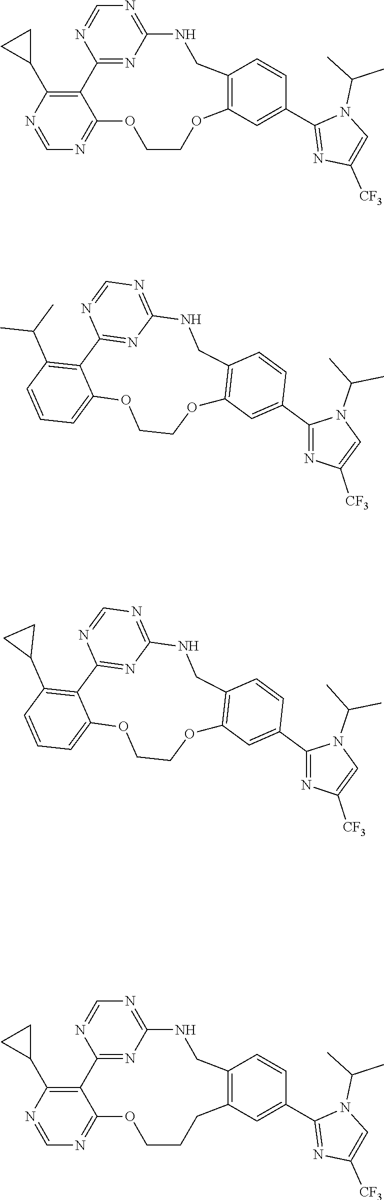

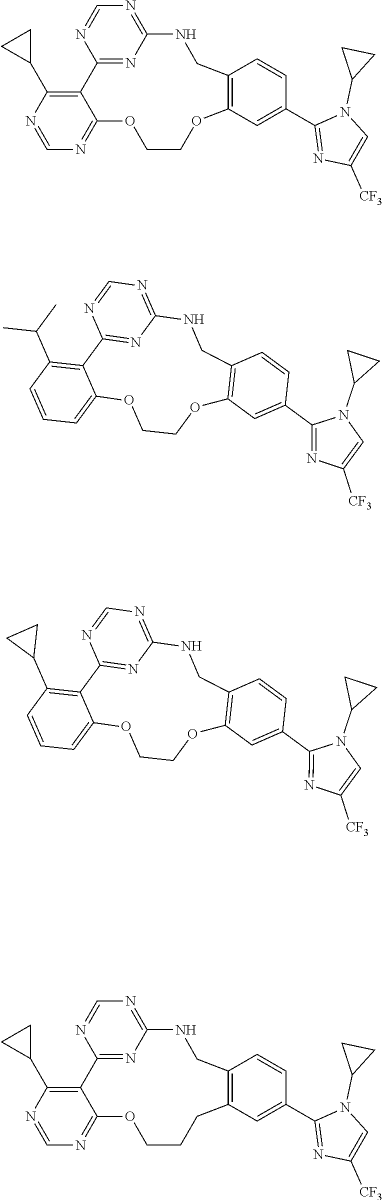 [SMILES: FC(F)(F)c1cn(C2CC2)c(-c2ccc3c(c2)OCCOc2ncnc(C4CC4)c2-c2ncnc(n2)NC3)n1 ; CC(C)c1cccc2c1-c1ncnc(n1)NCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncnc(n2)NC3)n1 ; FC(F)(F)c1cn(C2CC2)c(-c2ccc3c(c2)CCCOc2ncnc(C4CC4)c2-c2ncnc(n2)NC3)n1]

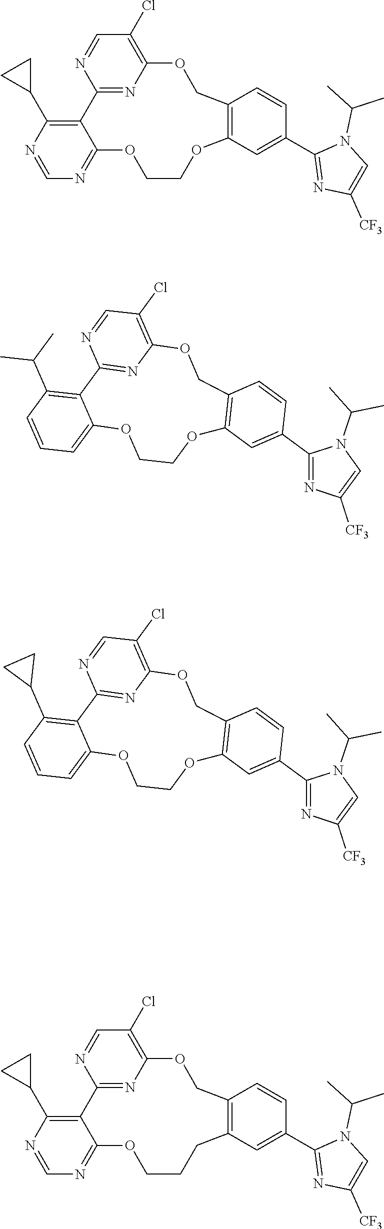

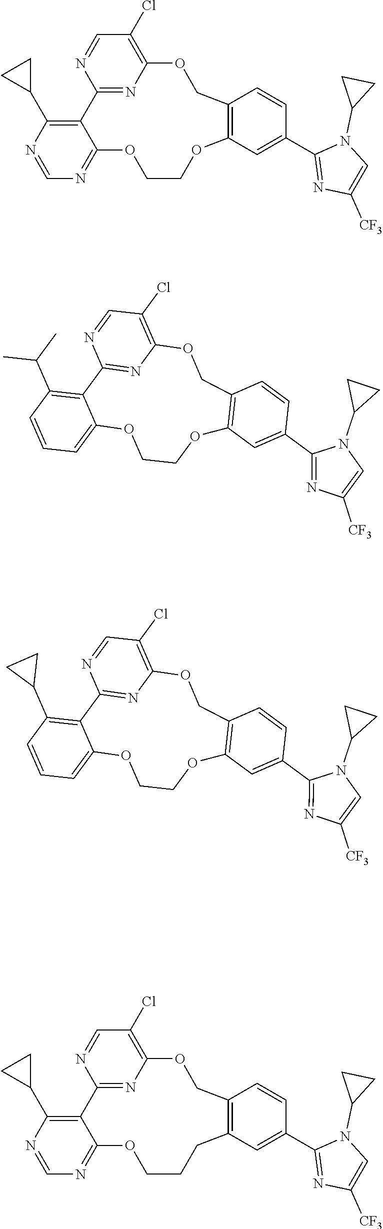 [SMILES: FC(F)(F)c1cn(C2CC2)c(-c2ccc3c(c2)OCCOc2ncnc(C4CC4)c2-c2ncc(Cl)c(n2)OC3)n1 ; CC(C)c1cccc2c1-c1ncc(Cl)c(n1)OCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncc(Cl)c(n2)OC3)n1 ; FC(F)(F)c1cn(C2CC2)c(-c2ccc3c(c2)CCCOc2ncnc(C4CC4)c2-c2ncc(Cl)c(n2)OC3)n1]

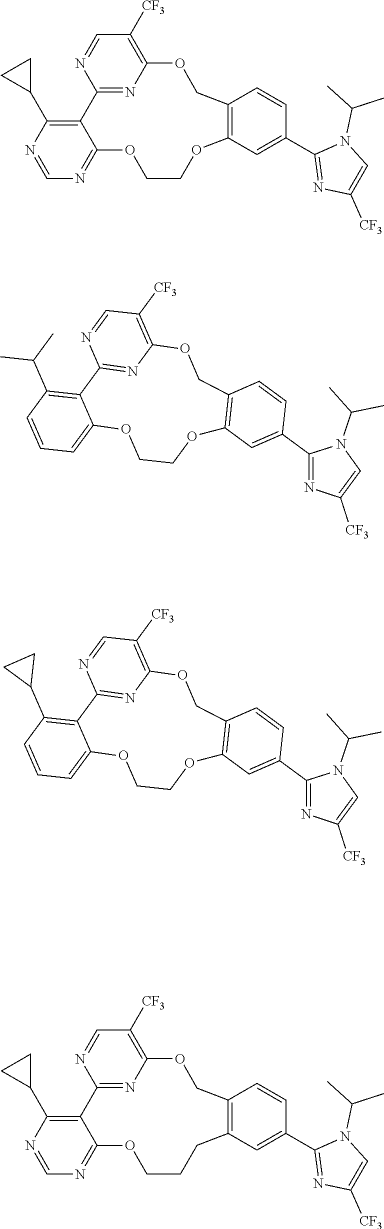

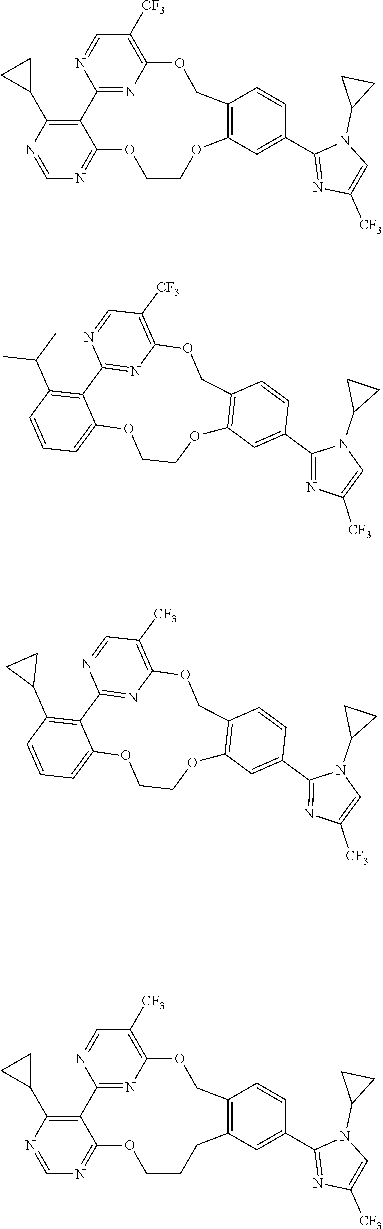 [SMILES: FC(F)(F)c1cn(C2CC2)c(-c2ccc3c(c2)OCCOc2ncnc(C4CC4)c2-c2ncc(C(F)(F)F)c(n2)OC3)n1 ; CC(C)c1cccc2c1-c1ncc(C(F)(F)F)c(n1)OCc1ccc(-c3nc(C(F)(F)F)cn3C3CC3)cc1OCCO2 ; FC(F)(F)c1cn(C2CC2)c(-c2ccc3c(c2)OCCOc2cccc(C4CC4)c2-c2ncc(C(F)(F)F)c(n2)OC3)n1 ; FC(F)(F)c1cn(C2CC2)c(-c2ccc3c(c2)CCCOc2ncnc(C4CC4)c2-c2ncc(C(F)(F)F)c(n2)OC3)n1]

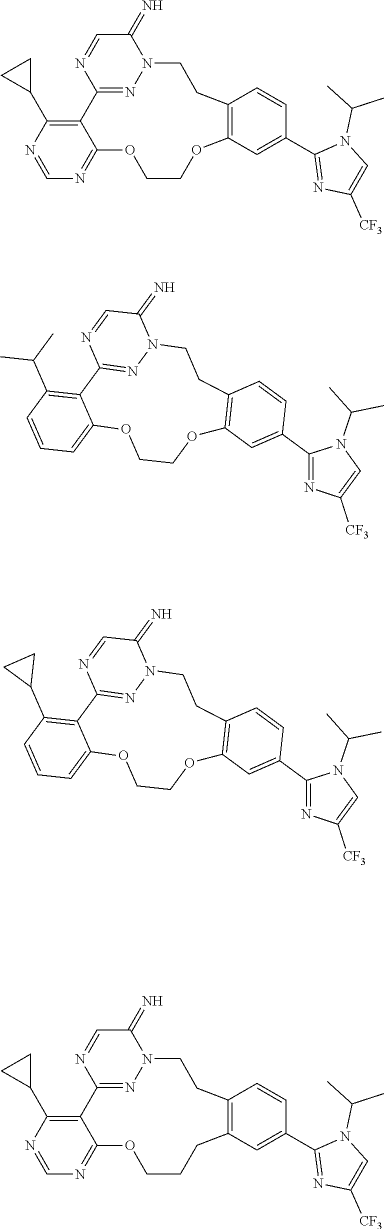

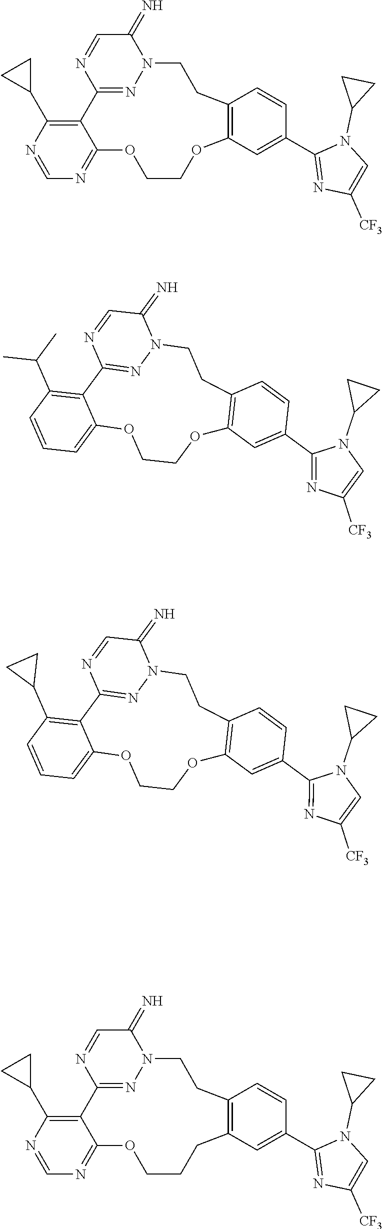 [SMILES: N=c1cnc2nn1CCc1ccc(-c3nc(C(F)(F)F)cn3C3CC3)cc1OCCOc1ncnc(C3CC3)c1-2 ; CC(C)c1cccc2c1-c1ncc(=N)n(n1)CCc1ccc(-c3nc(C(F)(F)F)cn3C3CC3)cc1OCCO2 ; N=c1cnc2nn1CCc1ccc(-c3nc(C(F)(F)F)cn3C3CC3)cc1OCCOc1cccc(C3CC3)c1-2 ; N=c1cnc2nn1CCc1ccc(-c3nc(C(F)(F)F)cn3C3CC3)cc1CCCOc1ncnc(C3CC3)c1-2]

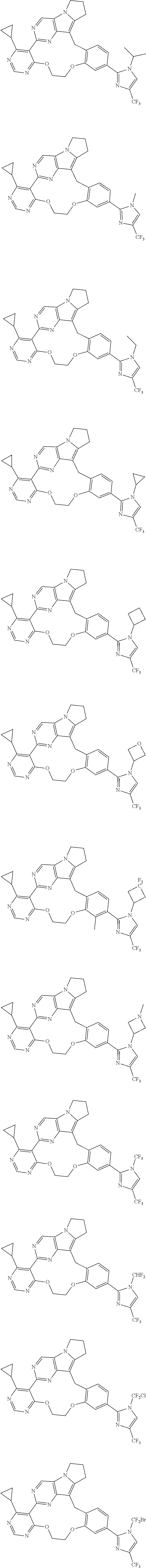

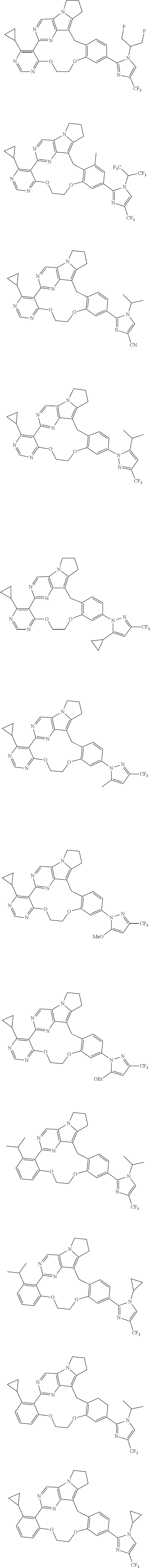

[chemical structure]

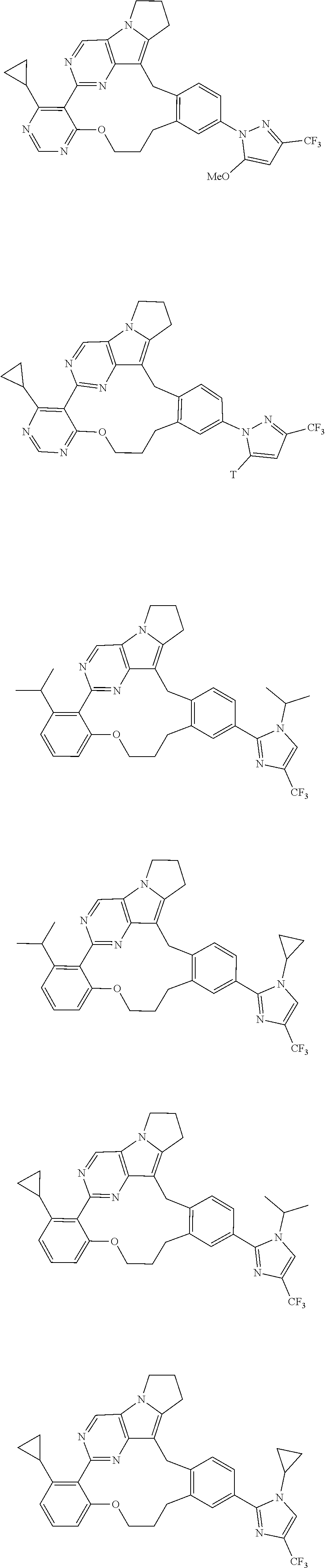

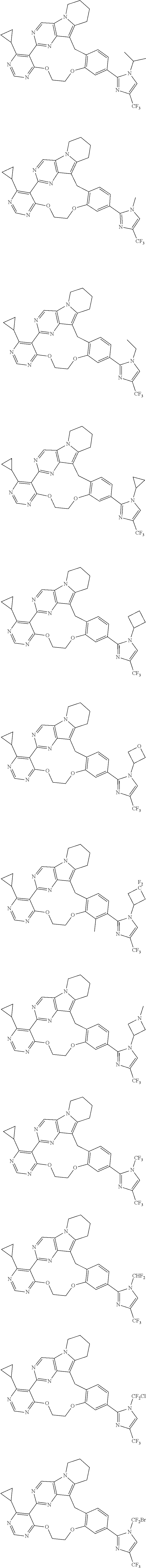

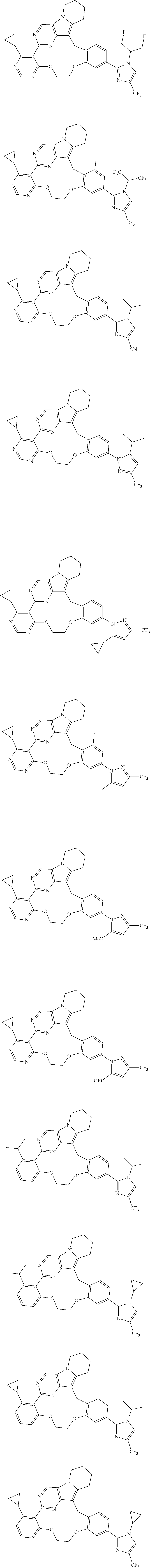

[chemical structure]

[chemical structure]

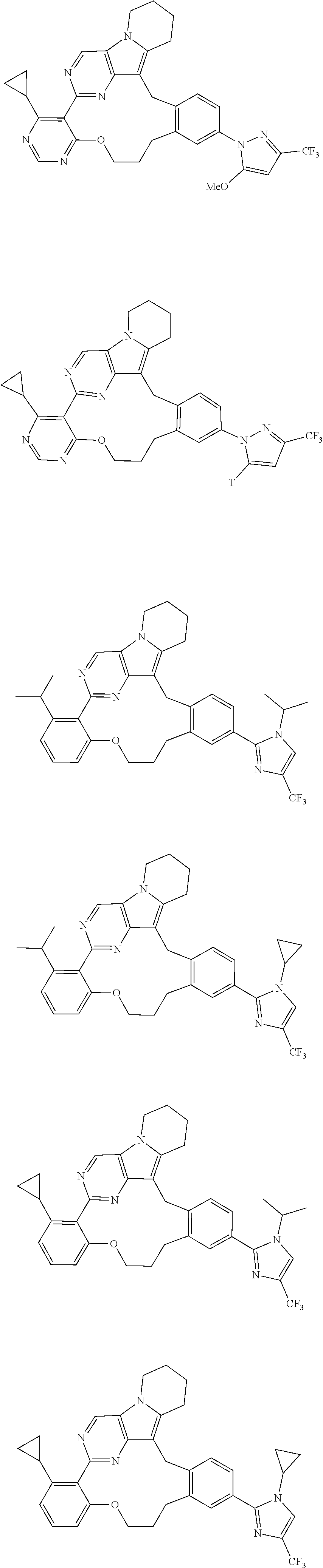

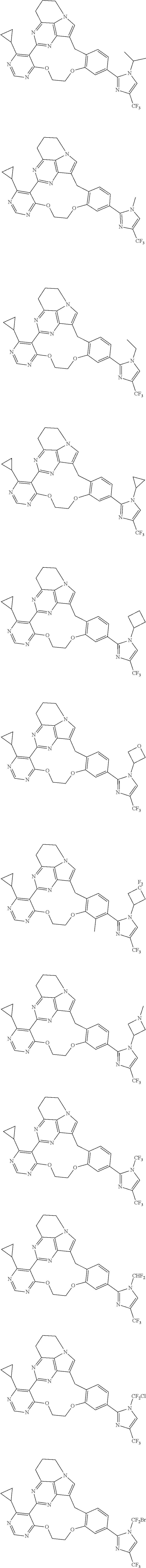

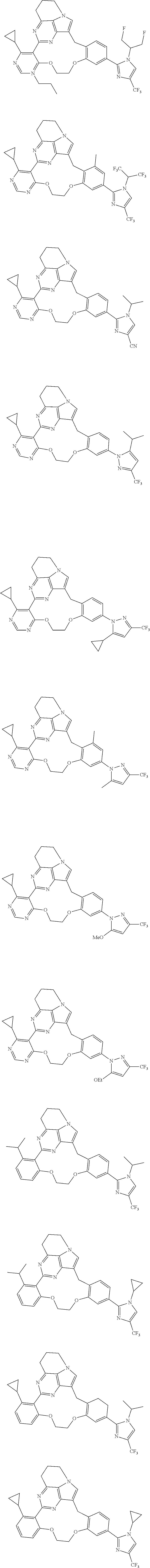

[chemical structure]

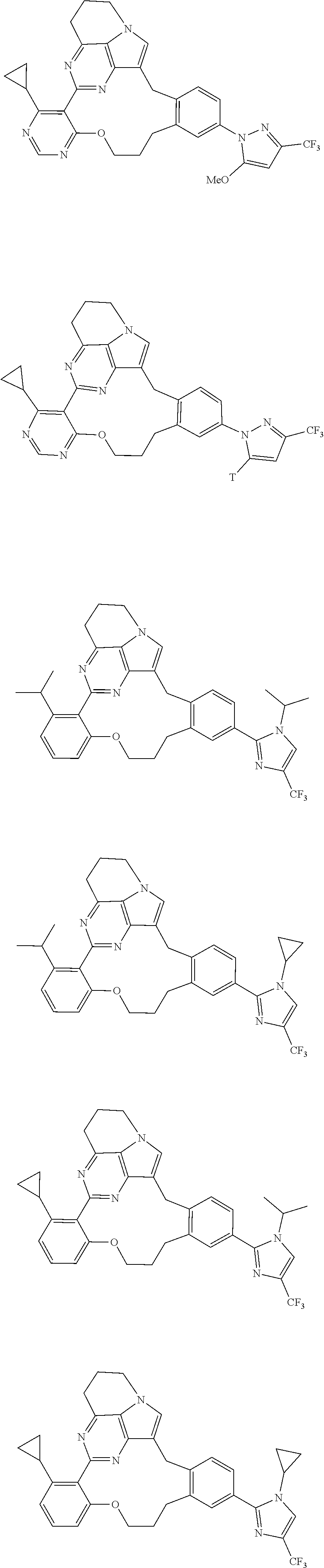

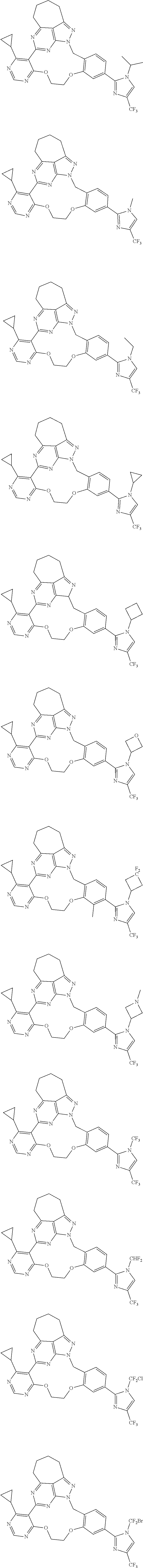

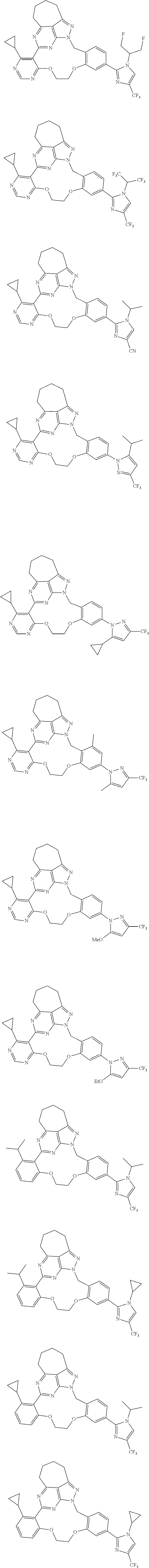

[chemical structure]

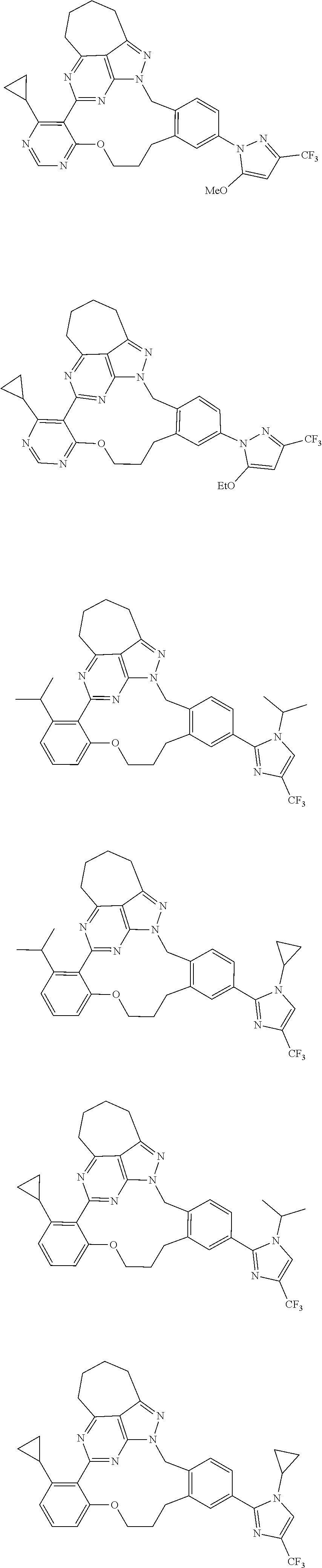

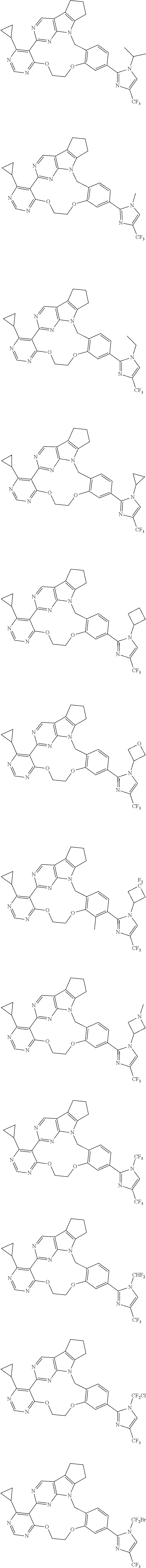

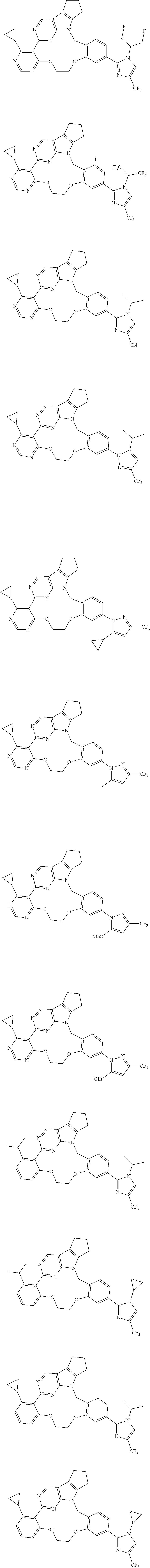

[chemical structure]

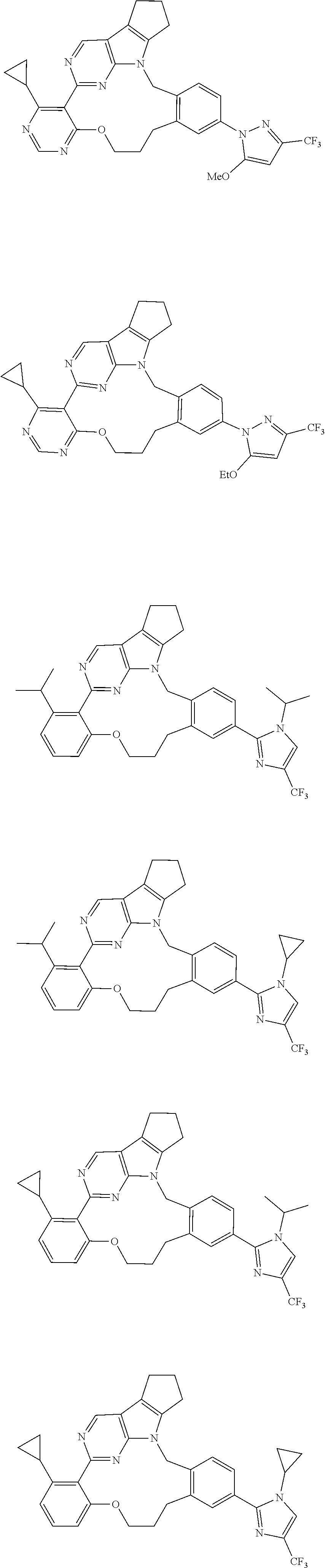

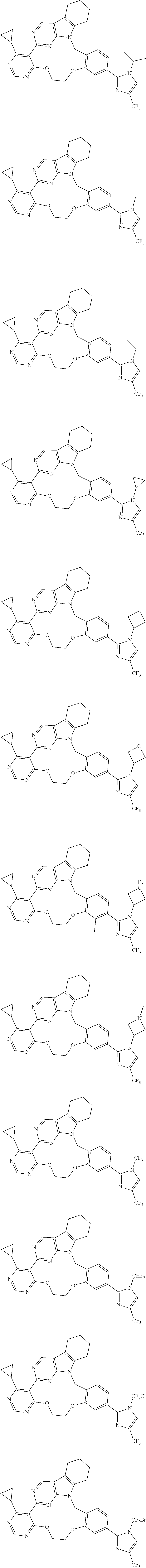

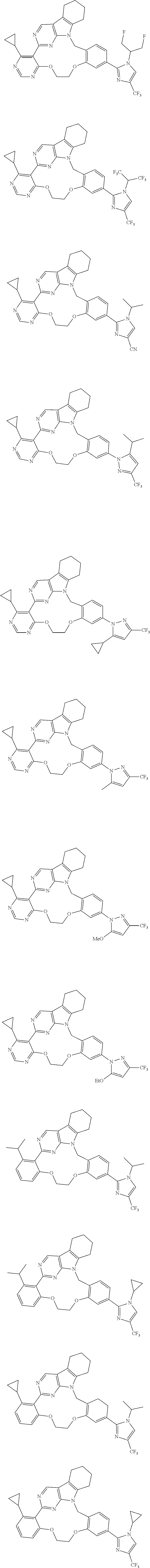

[chemical structure]

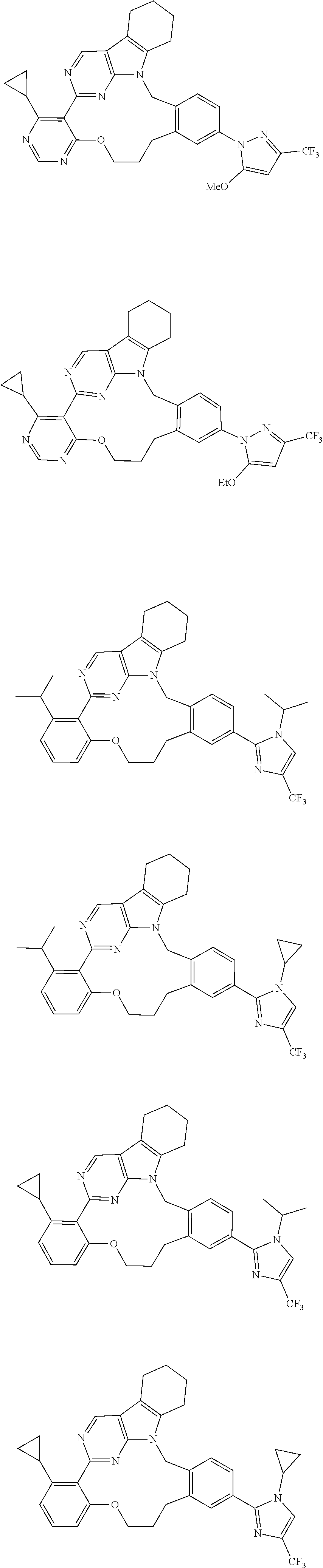

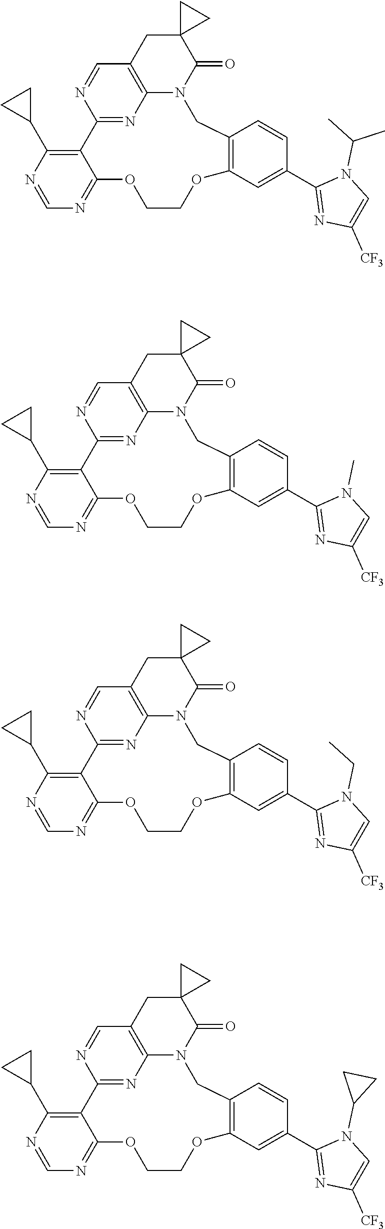 [SMILES: CC(C)n1cc(C(F)(F)F)nc1-c1ccc2c(c1)OCCOc1ncnc(C3CC3)c1-c1ncc3c(n1)N(C2)C(=O)C1(CC1)C3 ; Cn1cc(C(F)(F)F)nc1-c1ccc2c(c1)OCCOc1ncnc(C3CC3)c1-c1ncc3c(n1)N(C2)C(=O)C1(CC1)C3 ; CCn1cc(C(F)(F)F)nc1-c1ccc2c(c1)OCCOc1ncnc(C3CC3)c1-c1ncc3c(n1)N(C2)C(=O)C1(CC1)C3 ; O=C1N2Cc3ccc(-c4nc(C(F)(F)F)cn4C4CC4)cc3OCCOc3ncnc(C4CC4)c3-c3ncc(c2n3)CC12CC2]

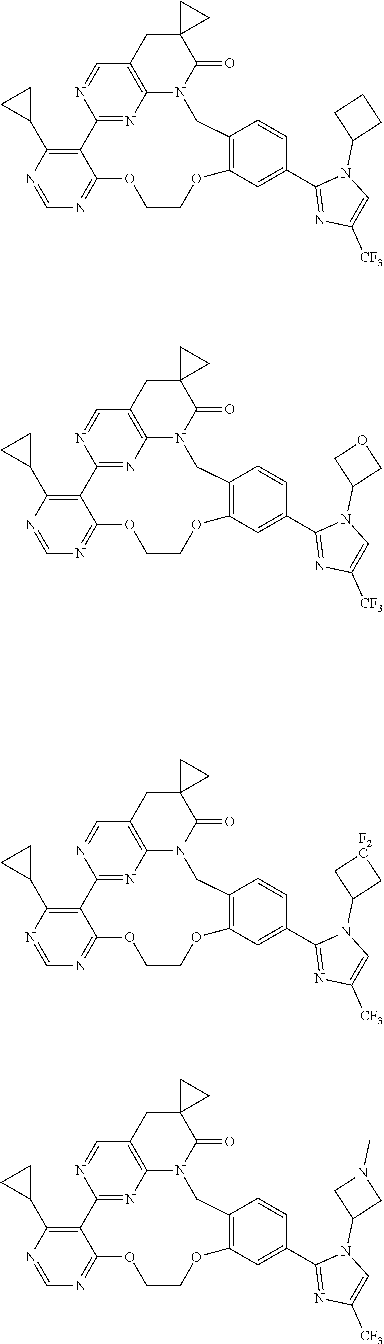 [SMILES: O=C1N2Cc3ccc(-c4nc(C(F)(F)F)cn4C4CCC4)cc3OCCOc3ncnc(C4CC4)c3-c3ncc(c2n3)CC12CC2 ; O=C1N2Cc3ccc(-c4nc(C(F)(F)F)cn4C4COC4)cc3OCCOc3ncnc(C4CC4)c3-c3ncc(c2n3)CC12CC2 ; O=C1N2Cc3ccc(-c4nc(C(F)(F)F)cn4C4CC(F)(F)C4)cc3OCCOc3ncnc(C4CC4)c3-c3ncc(c2n3)CC12CC2 ; CN1CC(n2cc(C(F)(F)F)nc2-c2ccc3c(c2)OCCOc2ncnc(C4CC4)c2-c2ncc4c(n2)N(C3)C(=O)C2(CC2)C4)C1]

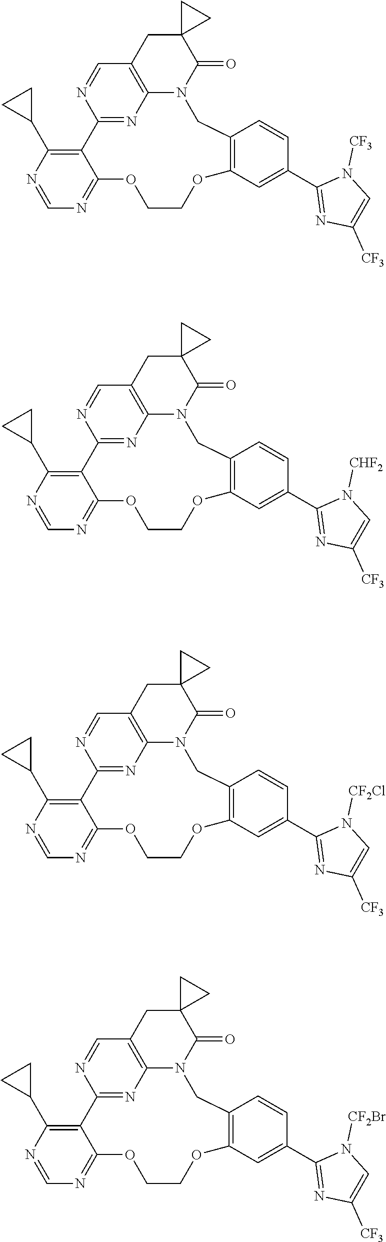

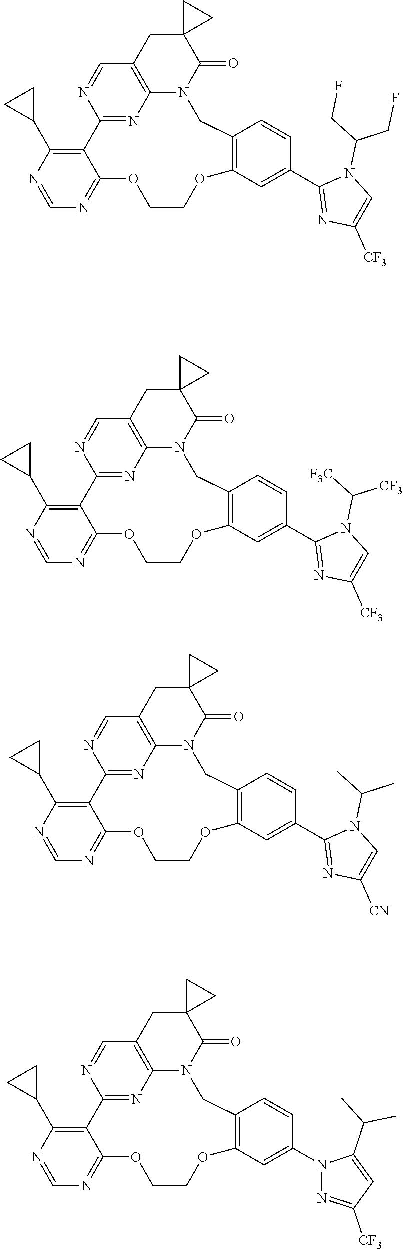 [SMILES: O=C1N2Cc3ccc(-c4nc(C(F)(F)F)cn4C(CF)CF)cc3OCCOc3ncnc(C4CC4)c3-c3ncc(c2n3)CC12CC2 ; O=C1N2Cc3ccc(-c4nc(C(F)(F)F)cn4C(C(F)(F)F)C(F)(F)F)cc3OCCOc3ncnc(C4CC4)c3-c3ncc(c2n3)CC12CC2 ; CC(C)n1cc(C#N)nc1-c1ccc2c(c1)OCCOc1ncnc(C3CC3)c1-c1ncc3c(n1)N(C2)C(=O)C1(CC1)C3 ; CC(C)c1cc(C(F)(F)F)nn1-c1ccc2c(c1)OCCOc1ncnc(C3CC3)c1-c1ncc3c(n1)N(C2)C(=O)C1(CC1)C3]

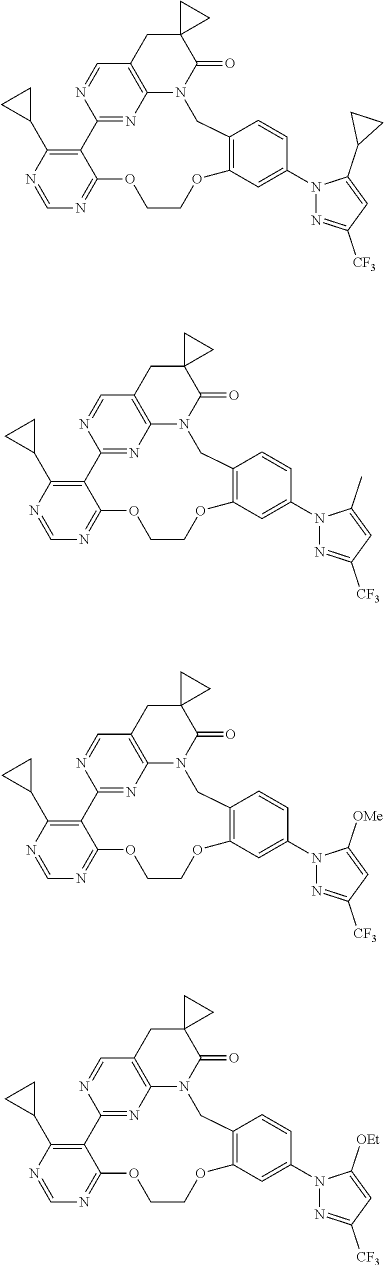 [SMILES: O=C1N2Cc3ccc(-n4nc(C(F)(F)F)cc4C4CC4)cc3OCCOc3ncnc(C4CC4)c3-c3ncc(c2n3)CC12CC2 ; Cc1cc(C(F)(F)F)nn1-c1ccc2c(c1)OCCOc1ncnc(C3CC3)c1-c1ncc3c(n1)N(C2)C(=O)C1(CC1)C3 ; COc1cc(C(F)(F)F)nn1-c1ccc2c(c1)OCCOc1ncnc(C3CC3)c1-c1ncc3c(n1)N(C2)C(=O)C1(CC1)C3 ; CCOc1cc(C(F)(F)F)nn1-c1ccc2c(c1)OCCOc1ncnc(C3CC3)c1-c1ncc3c(n1)N(C2)C(=O)C1(CC1)C3]

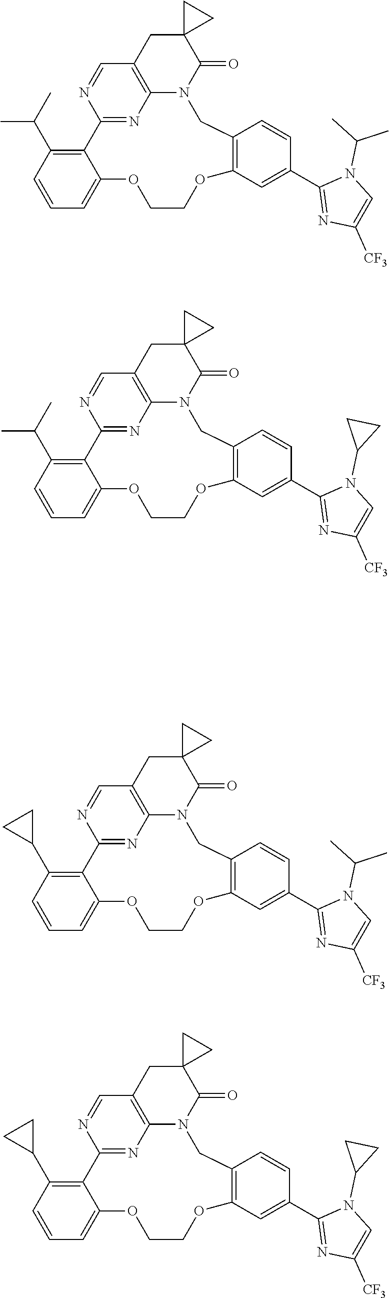

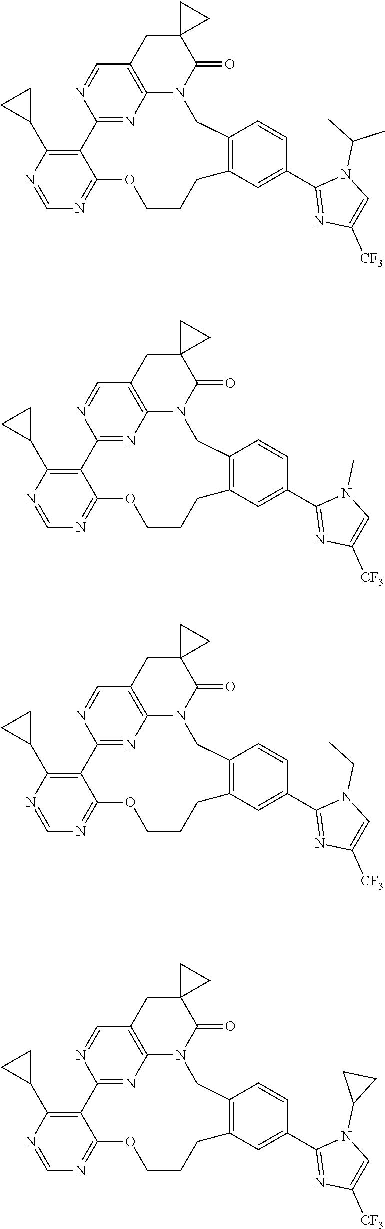 [SMILES: CC(C)n1cc(C(F)(F)F)nc1-c1ccc2c(c1)CCCOc1ncnc(C3CC3)c1-c1ncc3c(n1)N(C2)C(=O)C1(CC1)C3 ; Cn1cc(C(F)(F)F)nc1-c1ccc2c(c1)CCCOc1ncnc(C3CC3)c1-c1ncc3c(n1)N(C2)C(=O)C1(CC1)C3 ; CCn1cc(C(F)(F)F)nc1-c1ccc2c(c1)CCCOc1ncnc(C3CC3)c1-c1ncc3c(n1)N(C2)C(=O)C1(CC1)C3 ; O=C1N2Cc3ccc(-c4nc(C(F)(F)F)cn4C4CC4)cc3CCCOc3ncnc(C4CC4)c3-c3ncc(c2n3)CC12CC2]

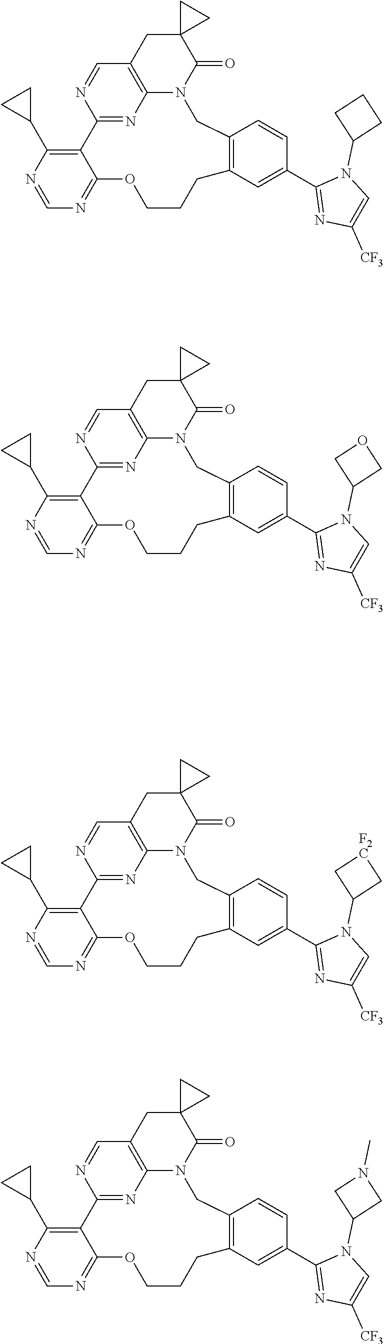 [SMILES: O=C1N2Cc3ccc(-c4nc(C(F)(F)F)cn4C4CCC4)cc3CCCOc3ncnc(C4CC4)c3-c3ncc(c2n3)CC12CC2 ; O=C1N2Cc3ccc(-c4nc(C(F)(F)F)cn4C4COC4)cc3CCCOc3ncnc(C4CC4)c3-c3ncc(c2n3)CC12CC2 ; O=C1N2Cc3ccc(-c4nc(C(F)(F)F)cn4C4CC(F)(F)C4)cc3CCCOc3ncnc(C4CC4)c3-c3ncc(c2n3)CC12CC2 ; CN1CC(n2cc(C(F)(F)F)nc2-c2ccc3c(c2)CCCOc2ncnc(C4CC4)c2-c2ncc4c(n2)N(C3)C(=O)C2(CC2)C4)C1]

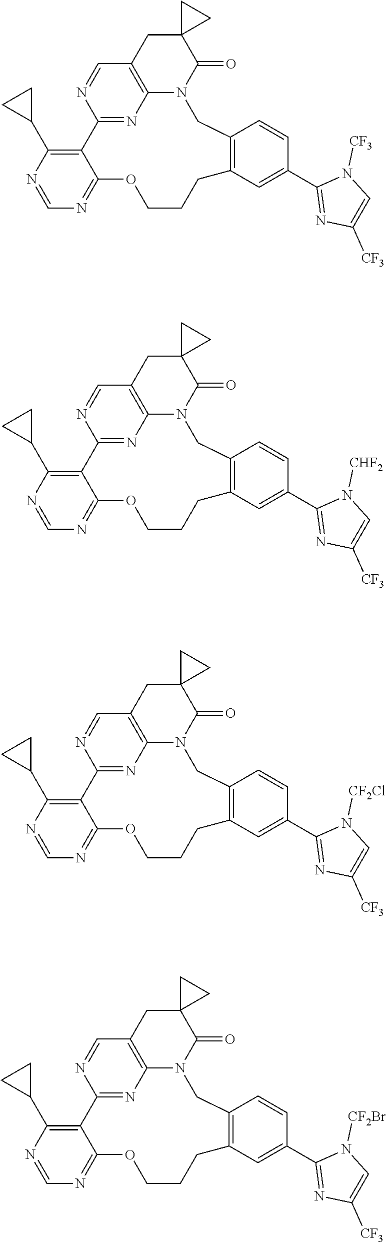

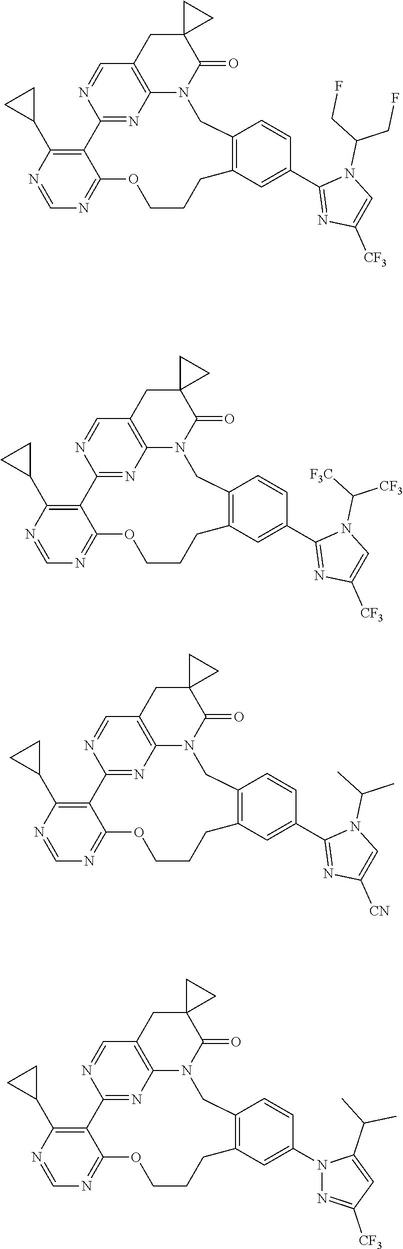 [SMILES: O=C1N2Cc3ccc(-c4nc(C(F)(F)F)cn4C(CF)CF)cc3CCCOc3ncnc(C4CC4)c3-c3ncc(c2n3)CC12CC2 ; O=C1N2Cc3ccc(-c4nc(C(F)(F)F)cn4C(C(F)(F)F)C(F)(F)F)cc3CCCOc3ncnc(C4CC4)c3-c3ncc(c2n3)CC12CC2 ; CC(C)n1cc(C#N)nc1-c1ccc2c(c1)CCCOc1ncnc(C3CC3)c1-c1ncc3c(n1)N(C2)C(=O)C1(CC1)C3 ; CC(C)c1cc(C(F)(F)F)nn1-c1ccc2c(c1)CCCOc1ncnc(C3CC3)c1-c1ncc3c(n1)N(C2)C(=O)C1(CC1)C3]

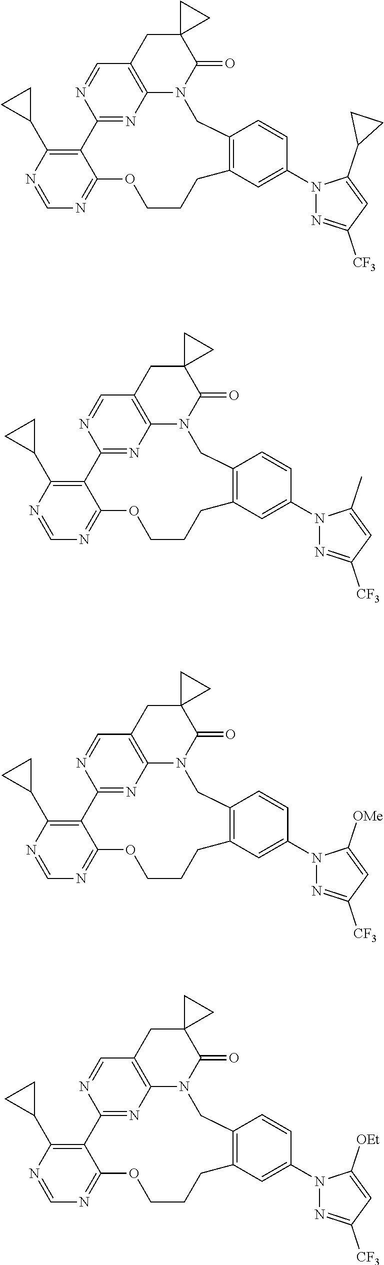 [SMILES: O=C1N2Cc3ccc(-n4nc(C(F)(F)F)cc4C4CC4)cc3CCCOc3ncnc(C4CC4)c3-c3ncc(c2n3)CC12CC2 ; Cc1cc(C(F)(F)F)nn1-c1ccc2c(c1)CCCOc1ncnc(C3CC3)c1-c1ncc3c(n1)N(C2)C(=O)C1(CC1)C3 ; COc1cc(C(F)(F)F)nn1-c1ccc2c(c1)CCCOc1ncnc(C3CC3)c1-c1ncc3c(n1)N(C2)C(=O)C1(CC1)C3 ; CCOc1cc(C(F)(F)F)nn1-c1ccc2c(c1)CCCOc1ncnc(C3CC3)c1-c1ncc3c(n1)N(C2)C(=O)C1(CC1)C3]

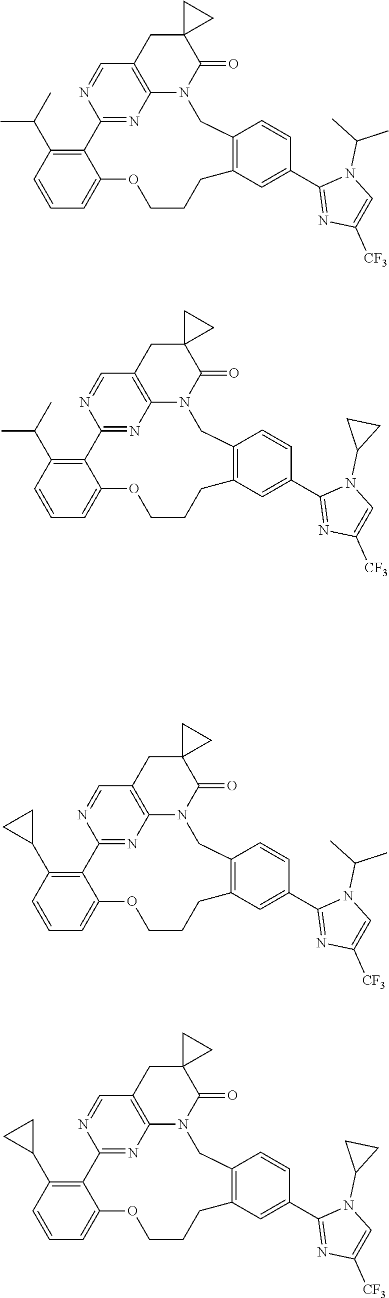

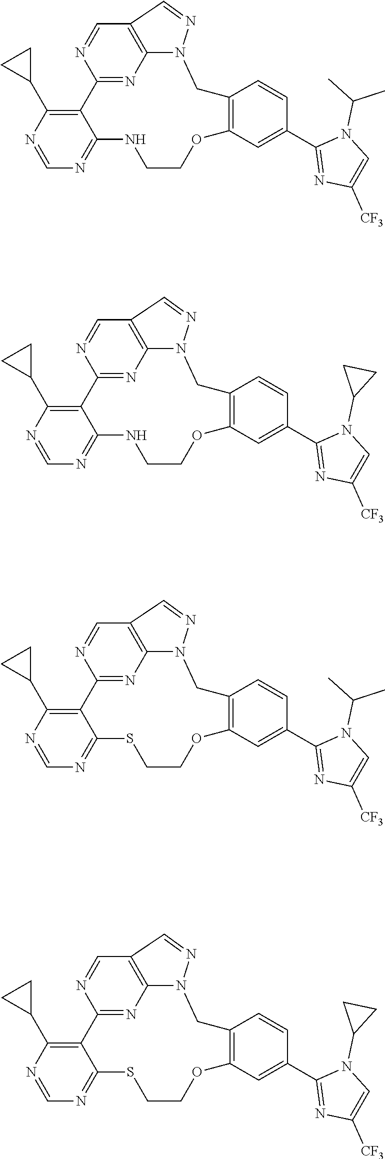 [SMILES: CC(C)n1cc(C(F)(F)F)nc1-c1ccc2c(c1)OCCNc1ncnc(C3CC3)c1-c1ncc3cnn(c3n1)C2 ; FC(F)(F)c1cn(C2CC2)c(-c2ccc3c(c2)OCCNc2ncnc(C4CC4)c2-c2ncc4cnn(c4n2)C3)n1 ; CC(C)n1cc(C(F)(F)F)nc1-c1ccc2c(c1)OCCSc1ncnc(C3CC3)c1-c1ncc3cnn(c3n1)C2 ; FC(F)(F)c1cn(C2CC2)c(-c2ccc3c(c2)OCCSc2ncnc(C4CC4)c2-c2ncc4cnn(c4n2)C3)n1]

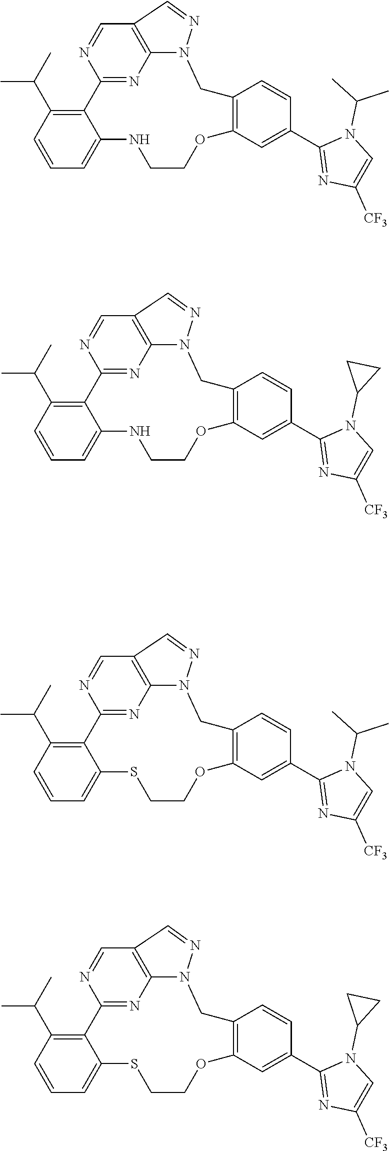

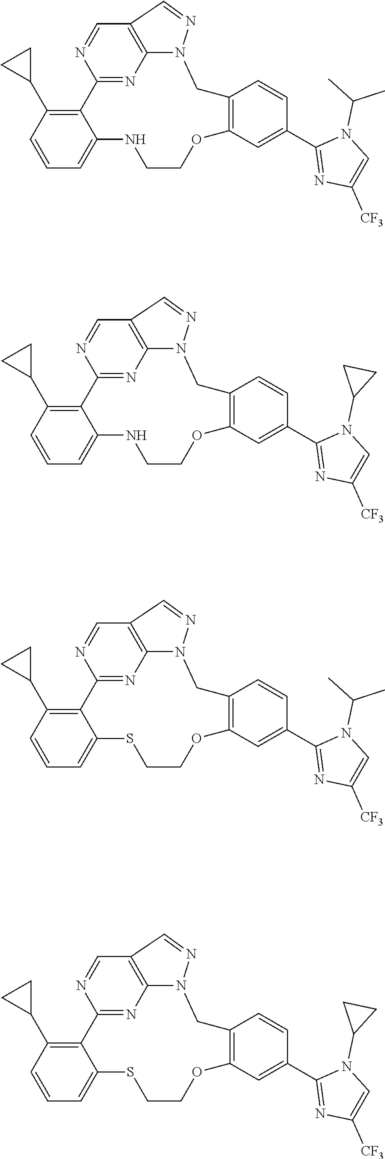 [SMILES: CC(C)n1cc(C(F)(F)F)nc1-c1ccc2c(c1)OCCNc1cccc(C3CC3)c1-c1ncc3cnn(c3n1)C2 ; FC(F)(F)c1cn(C2CC2)c(-c2ccc3c(c2)OCCNc2cccc(C4CC4)c2-c2ncc4cnn(c4n2)C3)n1 ; CC(C)n1cc(C(F)(F)F)nc1-c1ccc2c(c1)OCCSc1cccc(C3CC3)c1-c1ncc3cnn(c3n1)C2 ; FC(F)(F)c1cn(C2CC2)c(-c2ccc3c(c2)OCCSc2cccc(C4CC4)c2-c2ncc4cnn(c4n2)C3)n1]

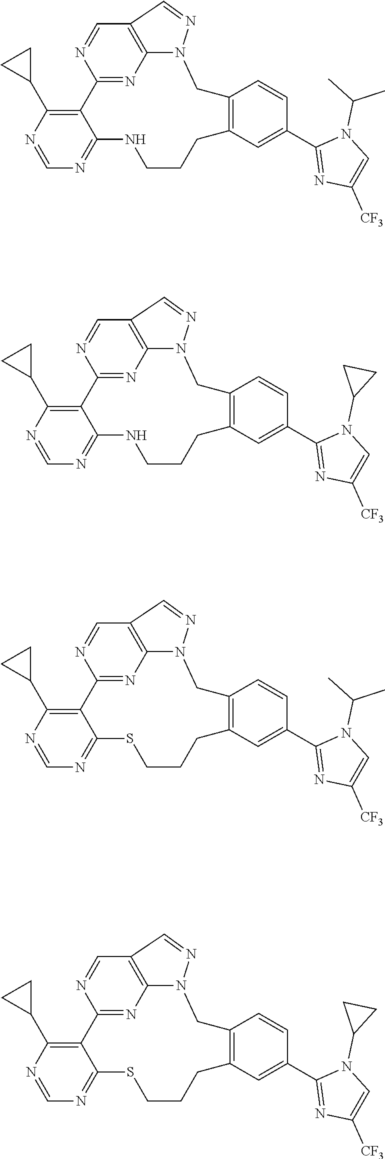 [SMILES: CC(C)n1cc(C(F)(F)F)nc1-c1ccc2c(c1)CCCNc1ncnc(C3CC3)c1-c1ncc3cnn(c3n1)C2 ; FC(F)(F)c1cn(C2CC2)c(-c2ccc3c(c2)CCCNc2ncnc(C4CC4)c2-c2ncc4cnn(c4n2)C3)n1 ; CC(C)n1cc(C(F)(F)F)nc1-c1ccc2c(c1)CCCSc1ncnc(C3CC3)c1-c1ncc3cnn(c3n1)C2 ; FC(F)(F)c1cn(C2CC2)c(-c2ccc3c(c2)CCCSc2ncnc(C4CC4)c2-c2ncc4cnn(c4n2)C3)n1]

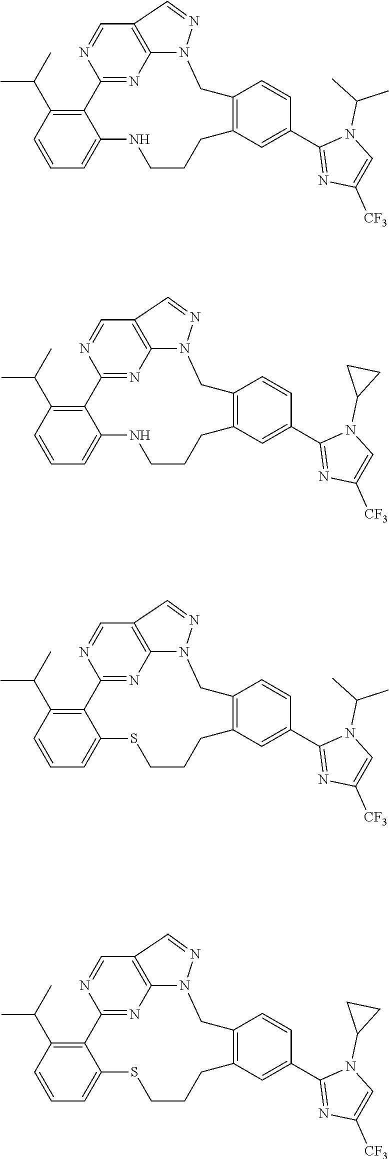

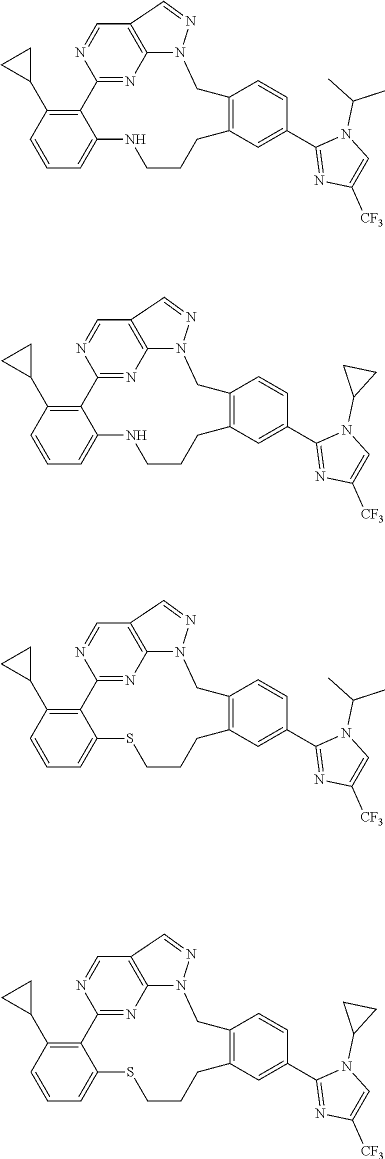 [SMILES: CC(C)n1cc(C(F)(F)F)nc1-c1ccc2c(c1)CCCNc1cccc(C3CC3)c1-c1ncc3cnn(c3n1)C2 ; FC(F)(F)c1cn(C2CC2)c(-c2ccc3c(c2)CCCNc2cccc(C4CC4)c2-c2ncc4cnn(c4n2)C3)n1 ; CC(C)n1cc(C(F)(F)F)nc1-c1ccc2c(c1)CCCSc1cccc(C3CC3)c1-c1ncc3cnn(c3n1)C2 ; FC(F)(F)c1cn(C2CC2)c(-c2ccc3c(c2)CCCSc2cccc(C4CC4)c2-c2ncc4cnn(c4n2)C3)n1]

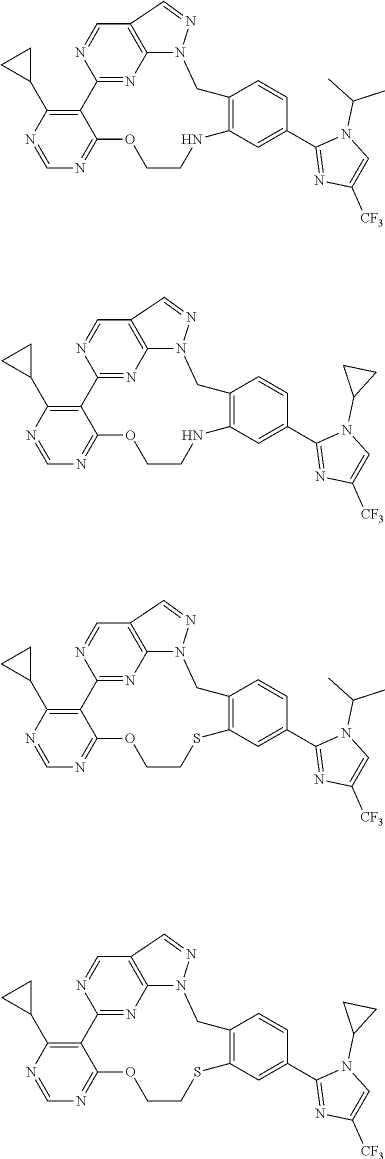 [SMILES: CC(C)n1cc(C(F)(F)F)nc1-c1ccc2c(c1)NCCOc1ncnc(C3CC3)c1-c1ncc3cnn(c3n1)C2 ; FC(F)(F)c1cn(C2CC2)c(-c2ccc3c(c2)NCCOc2ncnc(C4CC4)c2-c2ncc4cnn(c4n2)C3)n1 ; CC(C)n1cc(C(F)(F)F)nc1-c1ccc2c(c1)SCCOc1ncnc(C3CC3)c1-c1ncc3cnn(c3n1)C2 ; FC(F)(F)c1cn(C2CC2)c(-c2ccc3c(c2)SCCOc2ncnc(C4CC4)c2-c2ncc4cnn(c4n2)C3)n1]

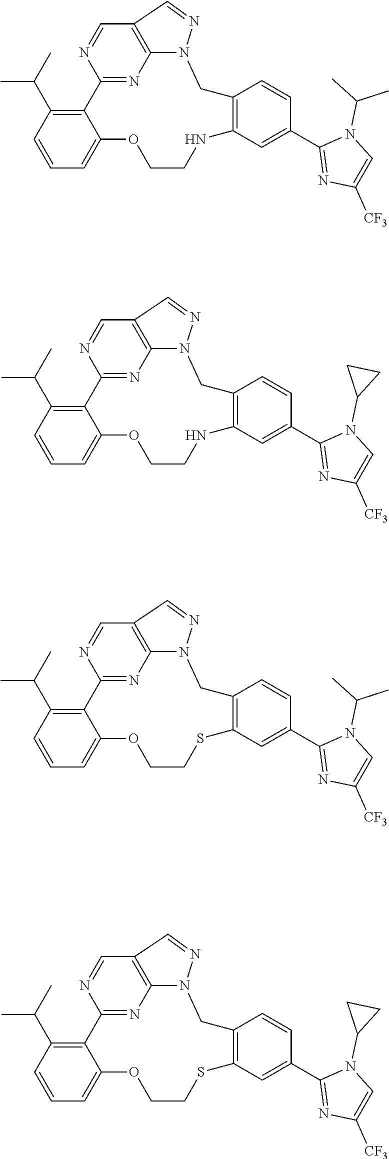

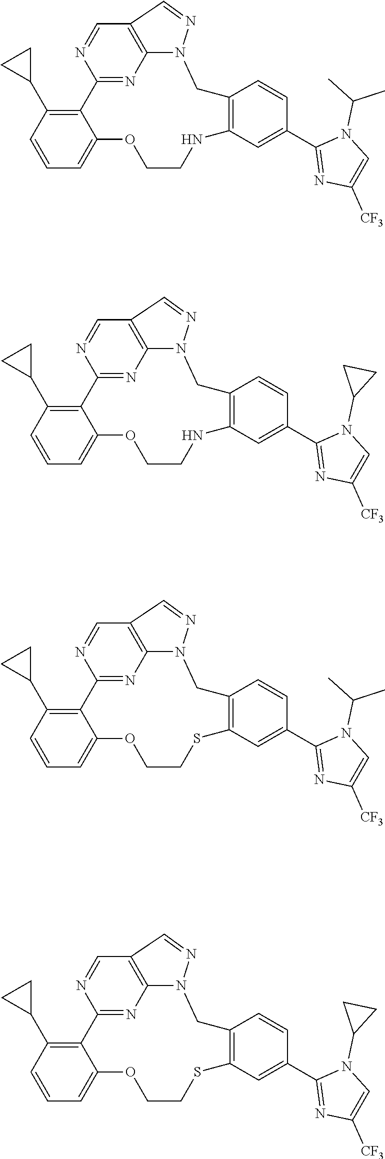 [SMILES: CC(C)n1cc(C(F)(F)F)nc1-c1ccc2c(c1)NCCOc1cccc(C3CC3)c1-c1ncc3cnn(c3n1)C2 ; FC(F)(F)c1cn(C2CC2)c(-c2ccc3c(c2)NCCOc2cccc(C4CC4)c2-c2ncc4cnn(c4n2)C3)n1 ; CC(C)n1cc(C(F)(F)F)nc1-c1ccc2c(c1)SCCOc1cccc(C3CC3)c1-c1ncc3cnn(c3n1)C2 ; FC(F)(F)c1cn(C2CC2)c(-c2ccc3c(c2)SCCOc2cccc(C4CC4)c2-c2ncc4cnn(c4n2)C3)n1]

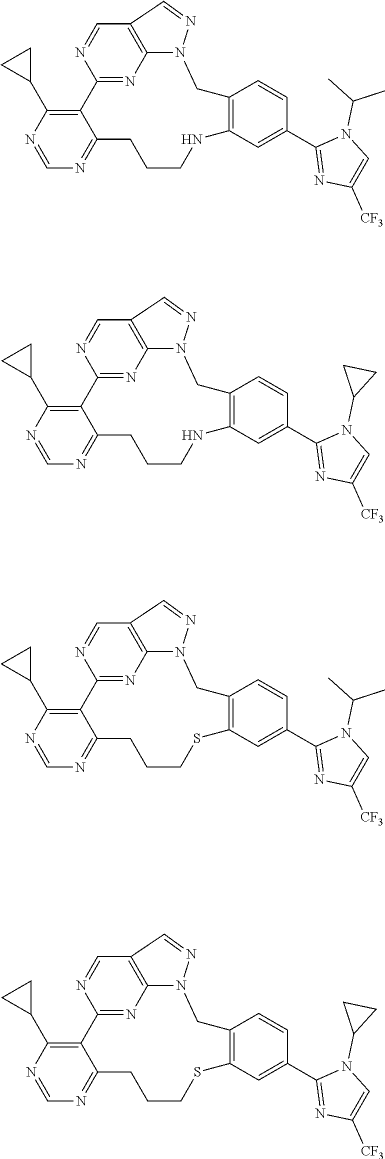 [SMILES: CC(C)n1cc(C(F)(F)F)nc1-c1ccc2c(c1)NCCCc1ncnc(C3CC3)c1-c1ncc3cnn(c3n1)C2 ; FC(F)(F)c1cn(C2CC2)c(-c2ccc3c(c2)NCCCc2ncnc(C4CC4)c2-c2ncc4cnn(c4n2)C3)n1 ; CC(C)n1cc(C(F)(F)F)nc1-c1ccc2c(c1)SCCCc1ncnc(C3CC3)c1-c1ncc3cnn(c3n1)C2 ; FC(F)(F)c1cn(C2CC2)c(-c2ccc3c(c2)SCCCc2ncnc(C4CC4)c2-c2ncc4cnn(c4n2)C3)n1]

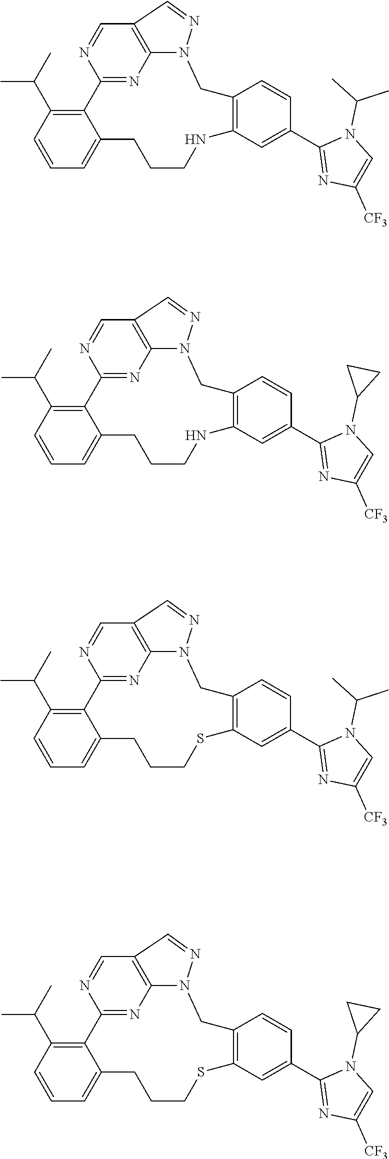

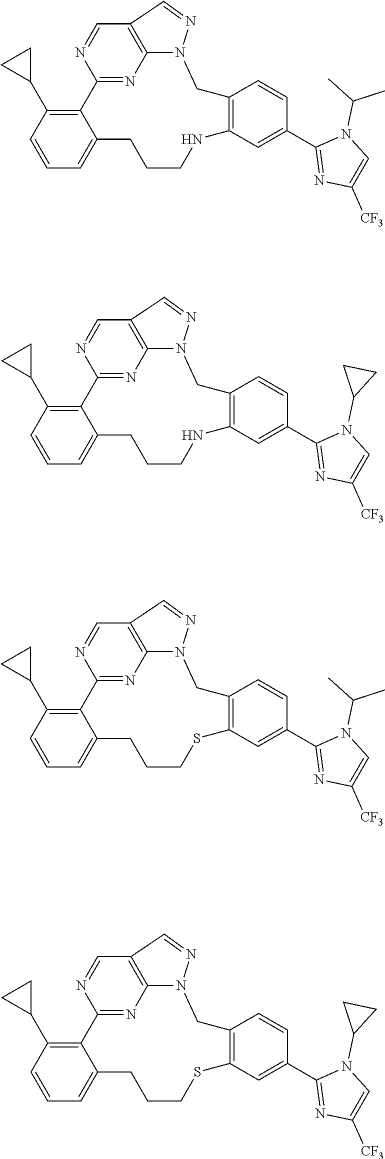 [SMILES: CC(C)n1cc(C(F)(F)F)nc1-c1ccc2c(c1)NCCCc1cccc(C3CC3)c1-c1ncc3cnn(c3n1)C2 ; FC(F)(F)c1cn(C2CC2)c(-c2ccc3c(c2)NCCCc2cccc(C4CC4)c2-c2ncc4cnn(c4n2)C3)n1 ; CC(C)n1cc(C(F)(F)F)nc1-c1ccc2c(c1)SCCCc1cccc(C3CC3)c1-c1ncc3cnn(c3n1)C2 ; FC(F)(F)c1cn(C2CC2)c(-c2ccc3c(c2)SCCCc2cccc(C4CC4)c2-c2ncc4cnn(c4n2)C3)n1]

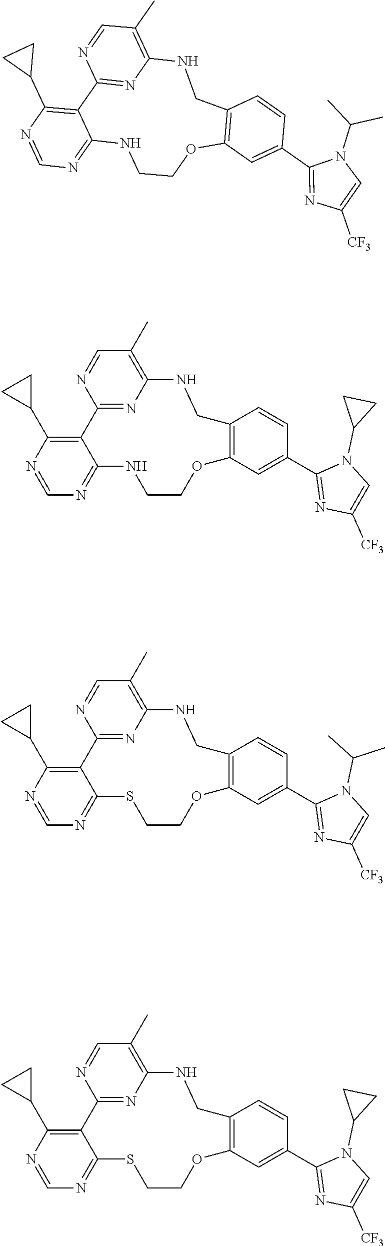 [SMILES: Cc1cnc2nc1NCc1ccc(-c3nc(C(F)(F)F)cn3C(C)C)cc1OCCNc1ncnc(C3CC3)c1-2 ; Cc1cnc2nc1NCc1ccc(-c3nc(C(F)(F)F)cn3C3CC3)cc1OCCNc1ncnc(C3CC3)c1-2 ; Cc1cnc2nc1NCc1ccc(-c3nc(C(F)(F)F)cn3C(C)C)cc1OCCSc1ncnc(C3CC3)c1-2 ; Cc1cnc2nc1NCc1ccc(-c3nc(C(F)(F)F)cn3C3CC3)cc1OCCSc1ncnc(C3CC3)c1-2]

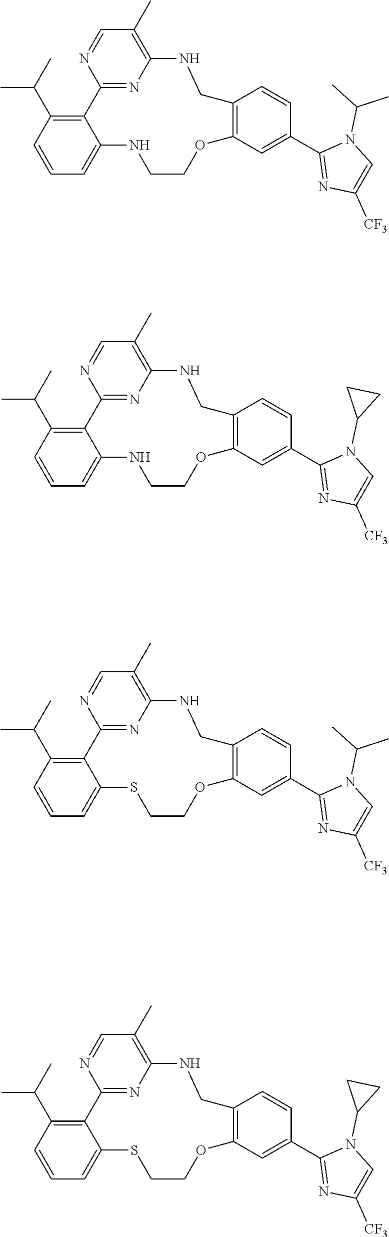

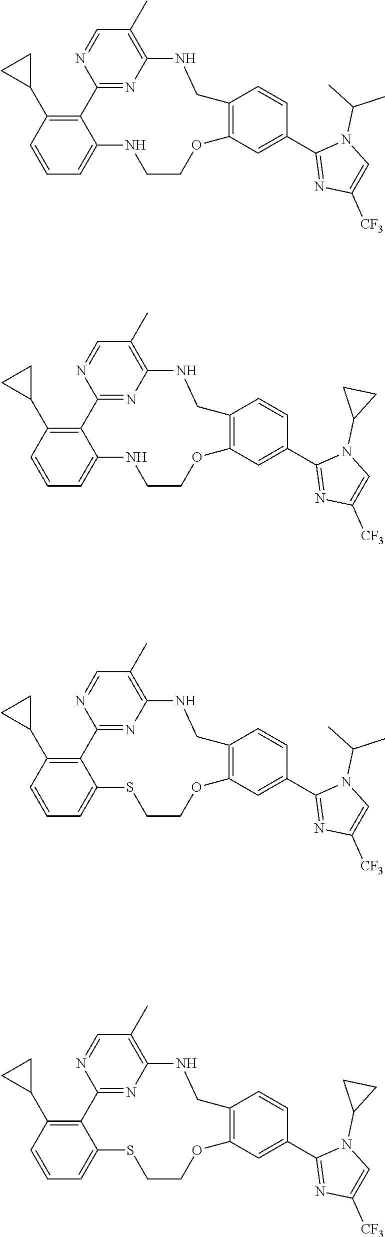 [SMILES: Cc1cnc2nc1NCc1ccc(-c3nc(C(F)(F)F)cn3C(C)C)cc1OCCNc1cccc(C3CC3)c1-2 ; Cc1cnc2nc1NCc1ccc(-c3nc(C(F)(F)F)cn3C3CC3)cc1OCCNc1cccc(C3CC3)c1-2 ; Cc1cnc2nc1NCc1ccc(-c3nc(C(F)(F)F)cn3C(C)C)cc1OCCSc1cccc(C3CC3)c1-2 ; Cc1cnc2nc1NCc1ccc(-c3nc(C(F)(F)F)cn3C3CC3)cc1OCCSc1cccc(C3CC3)c1-2]

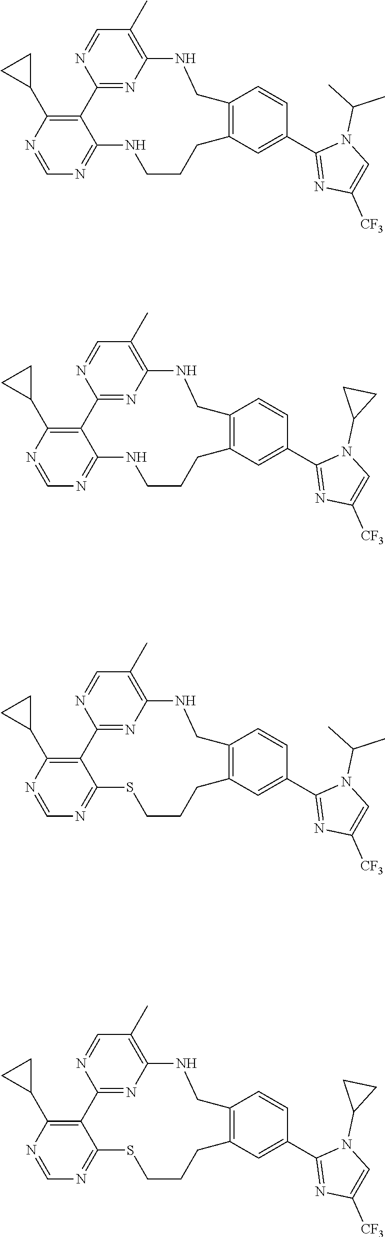 [SMILES: Cc1cnc2nc1NCc1ccc(-c3nc(C(F)(F)F)cn3C(C)C)cc1CCCNc1ncnc(C3CC3)c1-2 ; Cc1cnc2nc1NCc1ccc(-c3nc(C(F)(F)F)cn3C3CC3)cc1CCCNc1ncnc(C3CC3)c1-2 ; Cc1cnc2nc1NCc1ccc(-c3nc(C(F)(F)F)cn3C(C)C)cc1CCCSc1ncnc(C3CC3)c1-2 ; Cc1cnc2nc1NCc1ccc(-c3nc(C(F)(F)F)cn3C3CC3)cc1CCCSc1ncnc(C3CC3)c1-2]

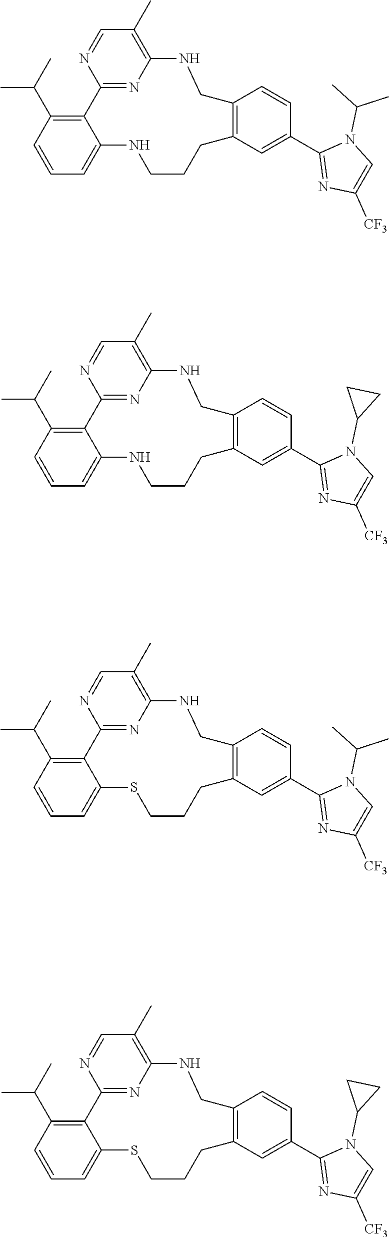

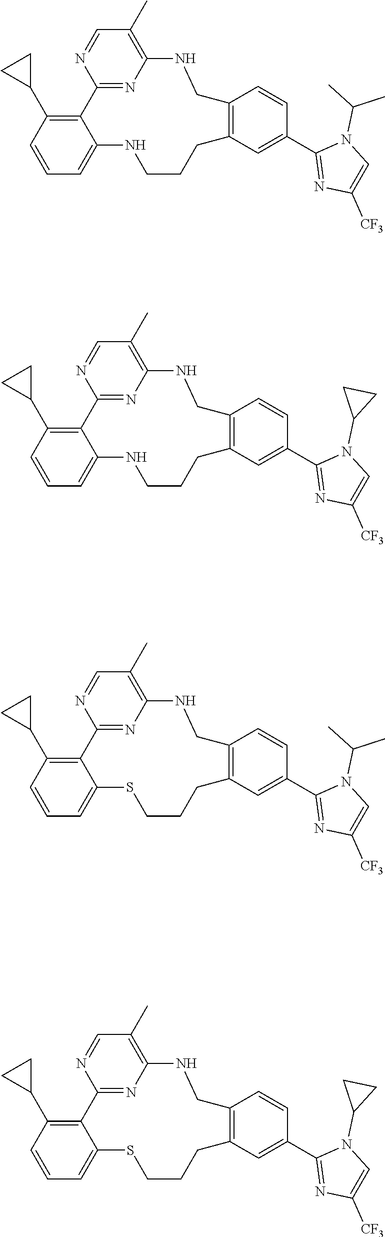 [SMILES: Cc1cnc2nc1NCc1ccc(-c3nc(C(F)(F)F)cn3C(C)C)cc1CCCNc1cccc(C3CC3)c1-2 ; Cc1cnc2nc1NCc1ccc(-c3nc(C(F)(F)F)cn3C3CC3)cc1CCCNc1cccc(C3CC3)c1-2 ; Cc1cnc2nc1NCc1ccc(-c3nc(C(F)(F)F)cn3C(C)C)cc1CCCSc1cccc(C3CC3)c1-2 ; Cc1cnc2nc1NCc1ccc(-c3nc(C(F)(F)F)cn3C3CC3)cc1CCCSc1cccc(C3CC3)c1-2]

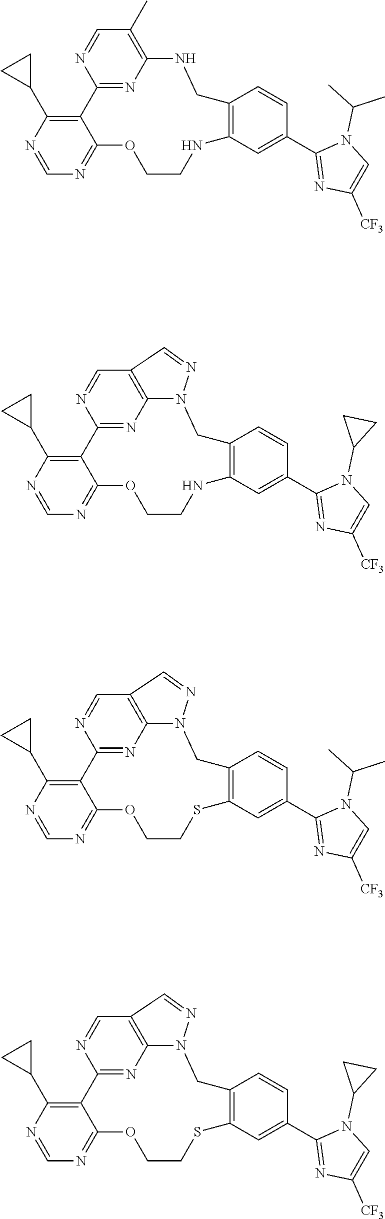 [SMILES: Cc1cnc2nc1NCc1ccc(-c3nc(C(F)(F)F)cn3C(C)C)cc1NCCOc1ncnc(C3CC3)c1-2 ; FC(F)(F)c1cn(C2CC2)c(-c2ccc3c(c2)NCCOc2ncnc(C4CC4)c2-c2ncc4cnn(c4n2)C3)n1 ; CC(C)n1cc(C(F)(F)F)nc1-c1ccc2c(c1)SCCOc1ncnc(C3CC3)c1-c1ncc3cnn(c3n1)C2 ; FC(F)(F)c1cn(C2CC2)c(-c2ccc3c(c2)SCCOc2ncnc(C4CC4)c2-c2ncc4cnn(c4n2)C3)n1]

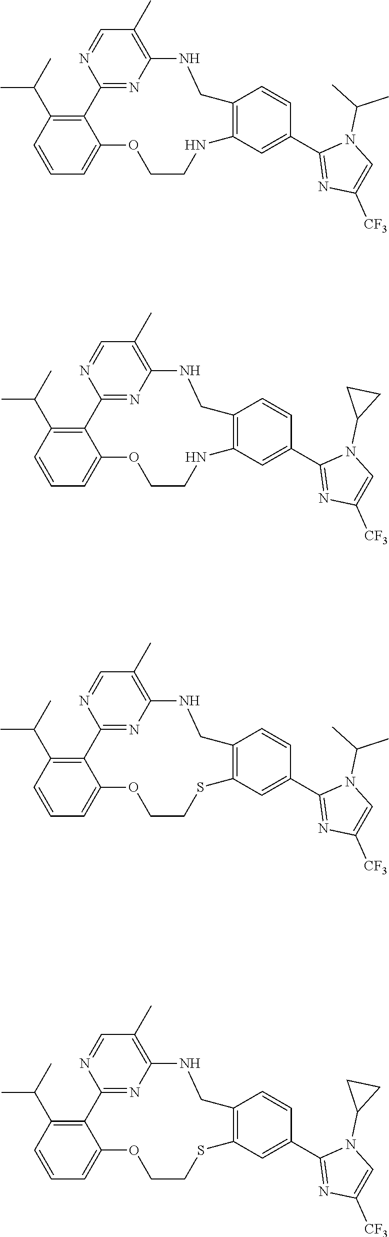

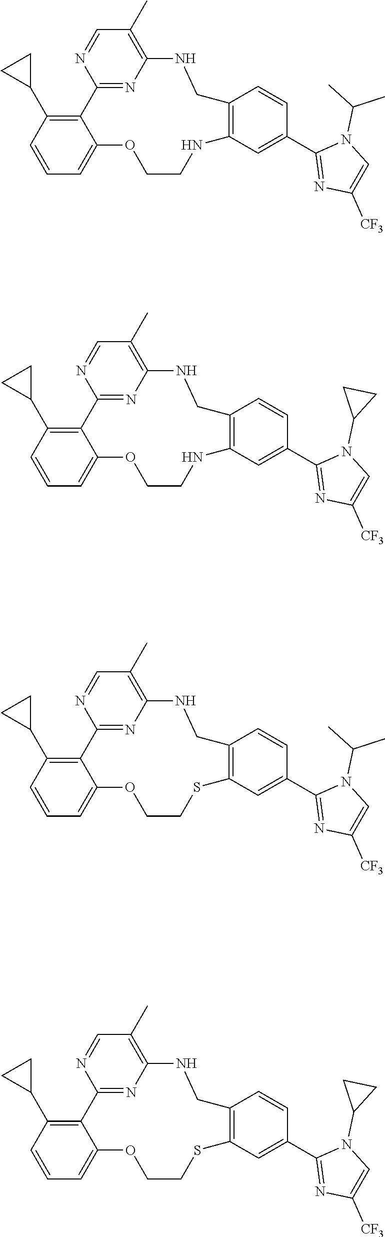 [SMILES: Cc1cnc2nc1NCc1ccc(-c3nc(C(F)(F)F)cn3C(C)C)cc1NCCOc1cccc(C3CC3)c1-2 ; Cc1cnc2nc1NCc1ccc(-c3nc(C(F)(F)F)cn3C3CC3)cc1NCCOc1cccc(C3CC3)c1-2 ; Cc1cnc2nc1NCc1ccc(-c3nc(C(F)(F)F)cn3C(C)C)cc1SCCOc1cccc(C3CC3)c1-2 ; Cc1cnc2nc1NCc1ccc(-c3nc(C(F)(F)F)cn3C3CC3)cc1SCCOc1cccc(C3CC3)c1-2]

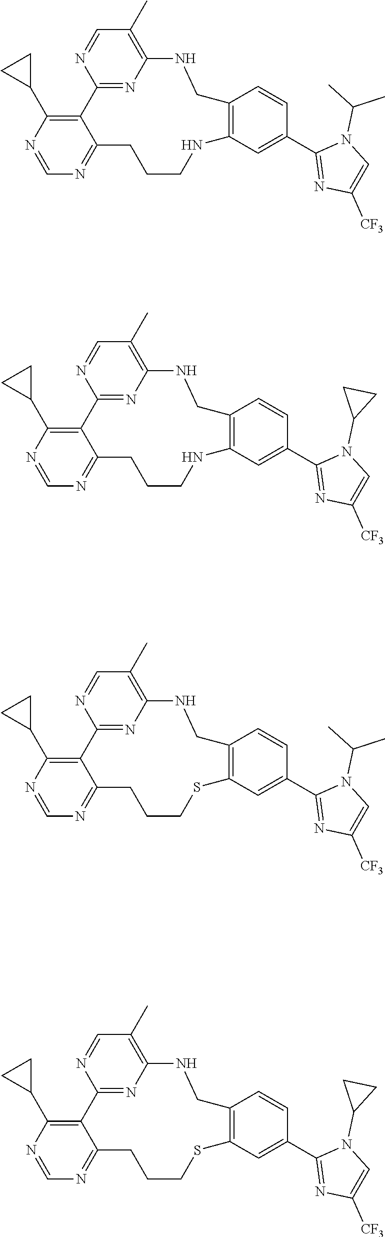 [SMILES: Cc1cnc2nc1NCc1ccc(-c3nc(C(F)(F)F)cn3C(C)C)cc1NCCCc1ncnc(C3CC3)c1-2 ; Cc1cnc2nc1NCc1ccc(-c3nc(C(F)(F)F)cn3C3CC3)cc1NCCCc1ncnc(C3CC3)c1-2 ; Cc1cnc2nc1NCc1ccc(-c3nc(C(F)(F)F)cn3C(C)C)cc1SCCCc1ncnc(C3CC3)c1-2 ; Cc1cnc2nc1NCc1ccc(-c3nc(C(F)(F)F)cn3C3CC3)cc1SCCCc1ncnc(C3CC3)c1-2]

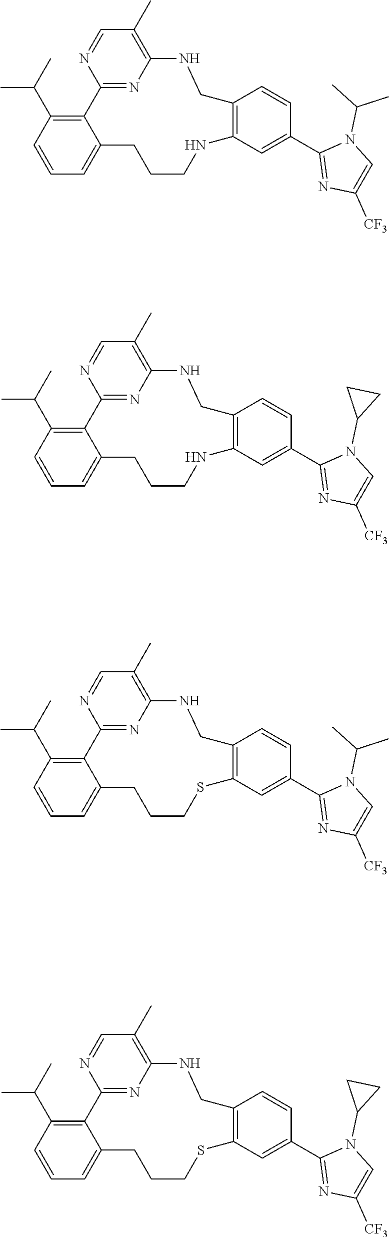

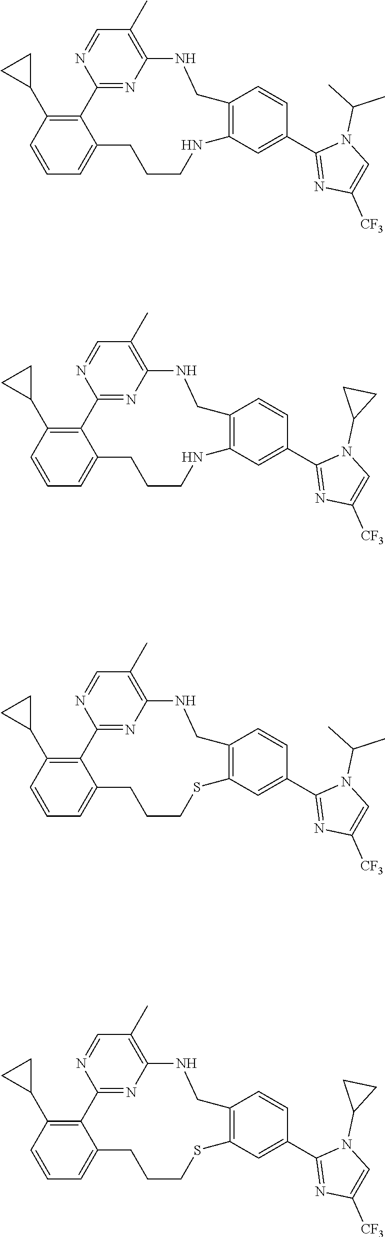 [SMILES: Cc1cnc2nc1NCc1ccc(-c3nc(C(F)(F)F)cn3C(C)C)cc1NCCCc1cccc(C3CC3)c1-2 ; Cc1cnc2nc1NCc1ccc(-c3nc(C(F)(F)F)cn3C3CC3)cc1NCCCc1cccc(C3CC3)c1-2 ; Cc1cnc2nc1NCc1ccc(-c3nc(C(F)(F)F)cn3C(C)C)cc1SCCCc1cccc(C3CC3)c1-2 ; Cc1cnc2nc1NCc1ccc(-c3nc(C(F)(F)F)cn3C3CC3)cc1SCCCc1cccc(C3CC3)c1-2]

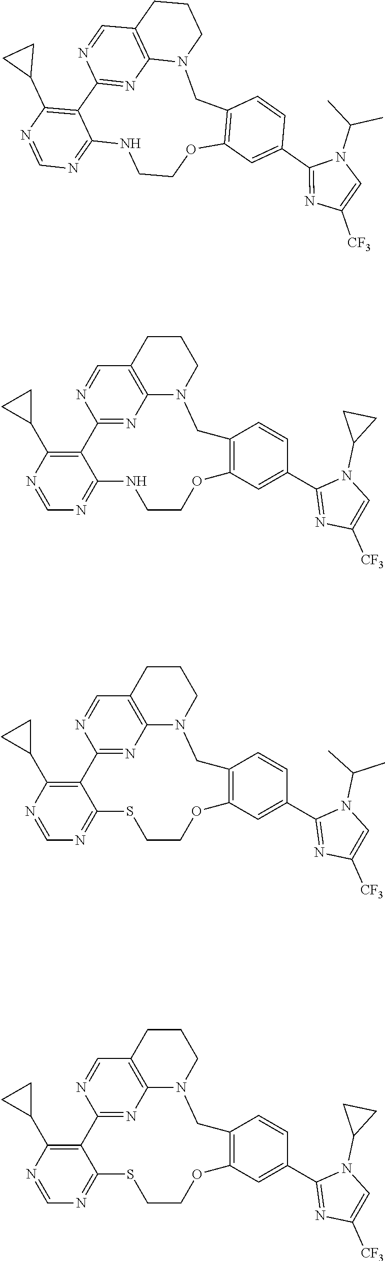 [SMILES: CC(C)n1cc(C(F)(F)F)nc1-c1ccc2c(c1)OCCNc1ncnc(C3CC3)c1-c1ncc3c(n1)N(CCC3)C2 ; FC(F)(F)c1cn(C2CC2)c(-c2ccc3c(c2)OCCNc2ncnc(C4CC4)c2-c2ncc4c(n2)N(CCC4)C3)n1 ; CC(C)n1cc(C(F)(F)F)nc1-c1ccc2c(c1)OCCSc1ncnc(C3CC3)c1-c1ncc3c(n1)N(CCC3)C2 ; FC(F)(F)c1cn(C2CC2)c(-c2ccc3c(c2)OCCSc2ncnc(C4CC4)c2-c2ncc4c(n2)N(CCC4)C3)n1]

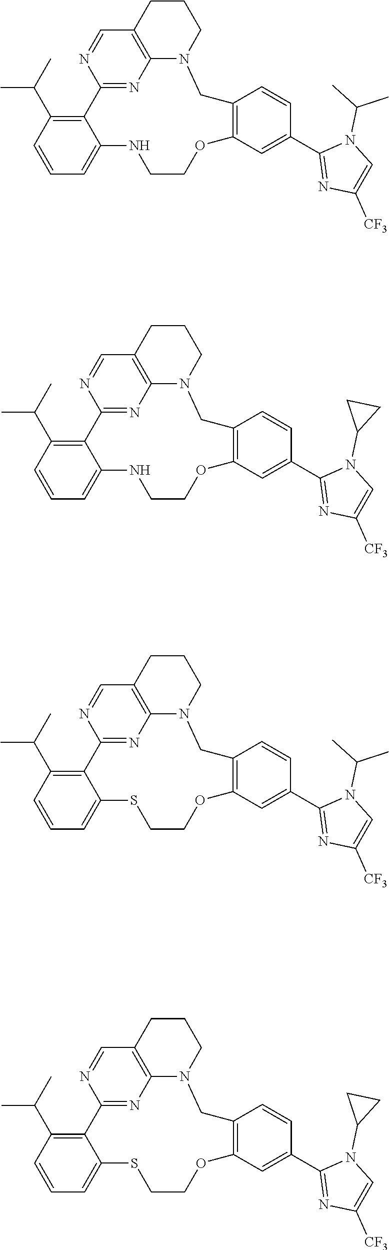

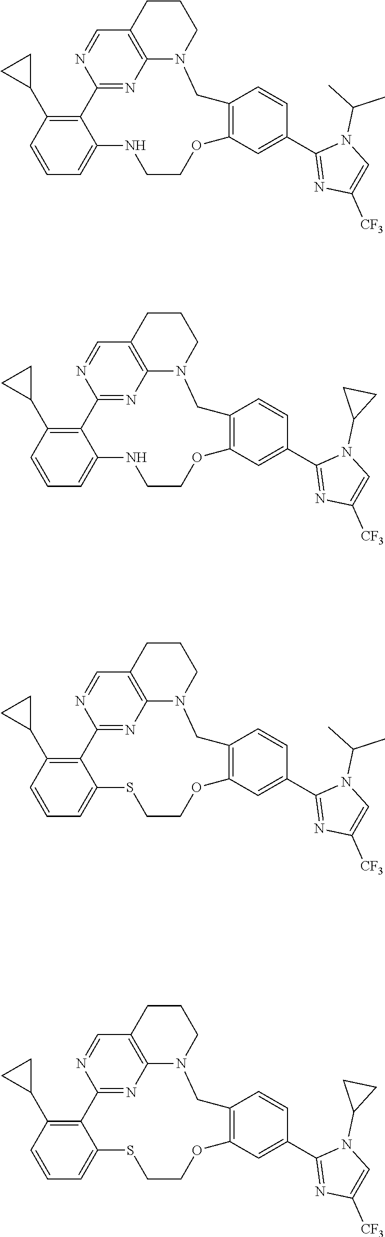 [SMILES: CC(C)n1cc(C(F)(F)F)nc1-c1ccc2c(c1)OCCNc1cccc(C3CC3)c1-c1ncc3c(n1)N(CCC3)C2 ; FC(F)(F)c1cn(C2CC2)c(-c2ccc3c(c2)OCCNc2cccc(C4CC4)c2-c2ncc4c(n2)N(CCC4)C3)n1 ; CC(C)n1cc(C(F)(F)F)nc1-c1ccc2c(c1)OCCSc1cccc(C3CC3)c1-c1ncc3c(n1)N(CCC3)C2 ; FC(F)(F)c1cn(C2CC2)c(-c2ccc3c(c2)OCCSc2cccc(C4CC4)c2-c2ncc4c(n2)N(CCC4)C3)n1]

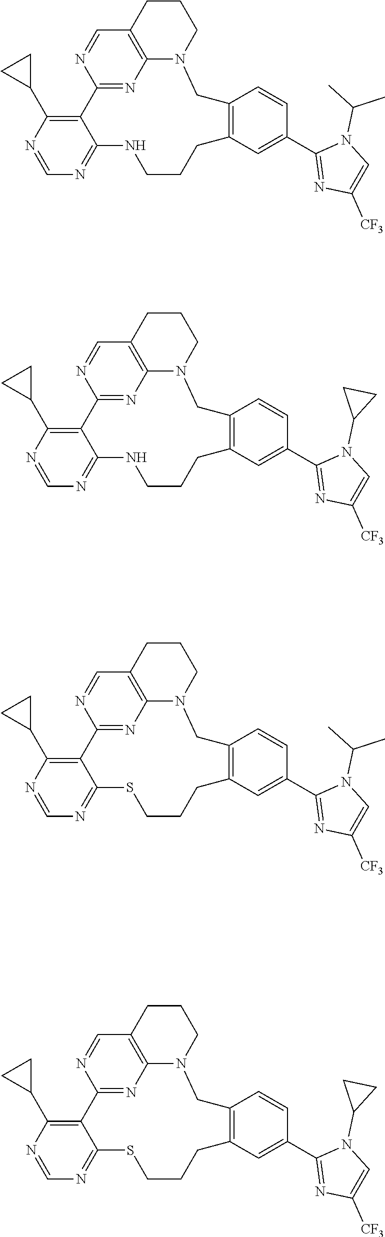 [SMILES: CC(C)n1cc(C(F)(F)F)nc1-c1ccc2c(c1)CCCNc1ncnc(C3CC3)c1-c1ncc3c(n1)N(CCC3)C2 ; FC(F)(F)c1cn(C2CC2)c(-c2ccc3c(c2)CCCNc2ncnc(C4CC4)c2-c2ncc4c(n2)N(CCC4)C3)n1 ; CC(C)n1cc(C(F)(F)F)nc1-c1ccc2c(c1)CCCSc1ncnc(C3CC3)c1-c1ncc3c(n1)N(CCC3)C2 ; FC(F)(F)c1cn(C2CC2)c(-c2ccc3c(c2)CCCSc2ncnc(C4CC4)c2-c2ncc4c(n2)N(CCC4)C3)n1]

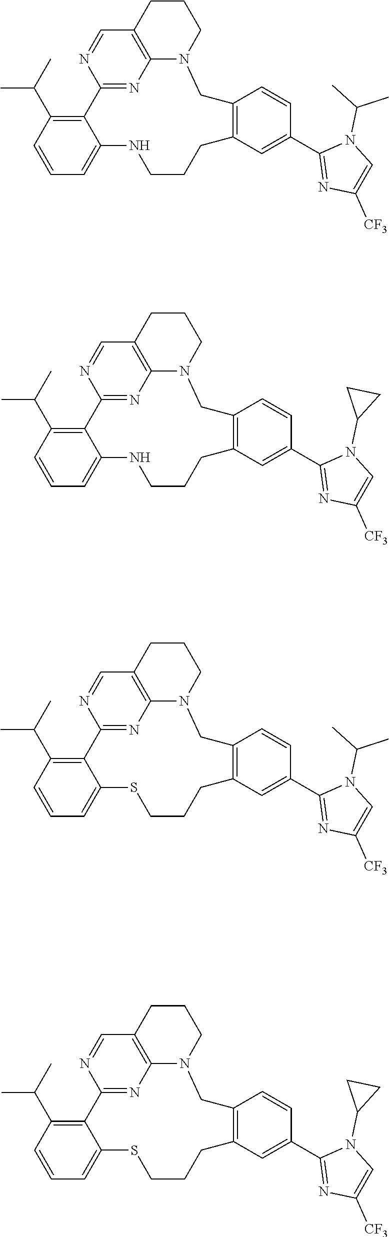

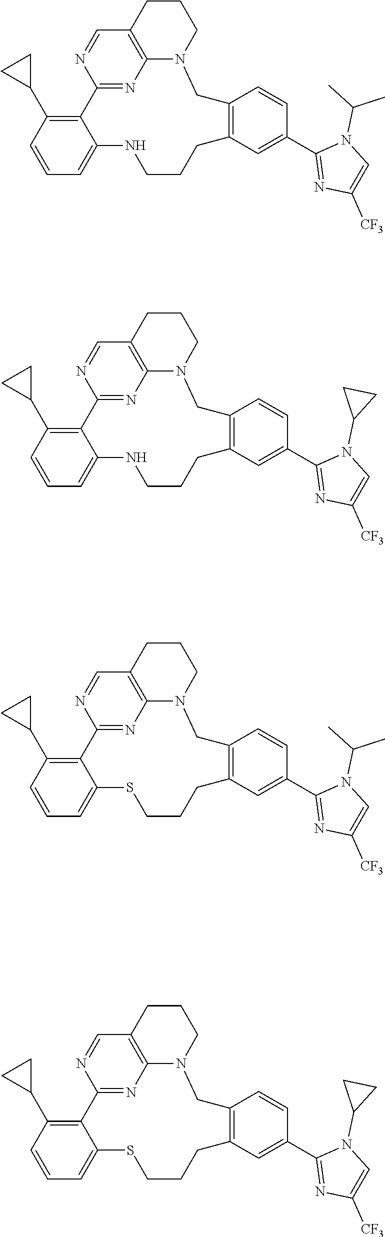 [SMILES: CC(C)n1cc(C(F)(F)F)nc1-c1ccc2c(c1)CCCNc1cccc(C3CC3)c1-c1ncc3c(n1)N(CCC3)C2 ; FC(F)(F)c1cn(C2CC2)c(-c2ccc3c(c2)CCCNc2cccc(C4CC4)c2-c2ncc4c(n2)N(CCC4)C3)n1 ; CC(C)n1cc(C(F)(F)F)nc1-c1ccc2c(c1)CCCSc1cccc(C3CC3)c1-c1ncc3c(n1)N(CCC3)C2 ; FC(F)(F)c1cn(C2CC2)c(-c2ccc3c(c2)CCCSc2cccc(C4CC4)c2-c2ncc4c(n2)N(CCC4)C3)n1]

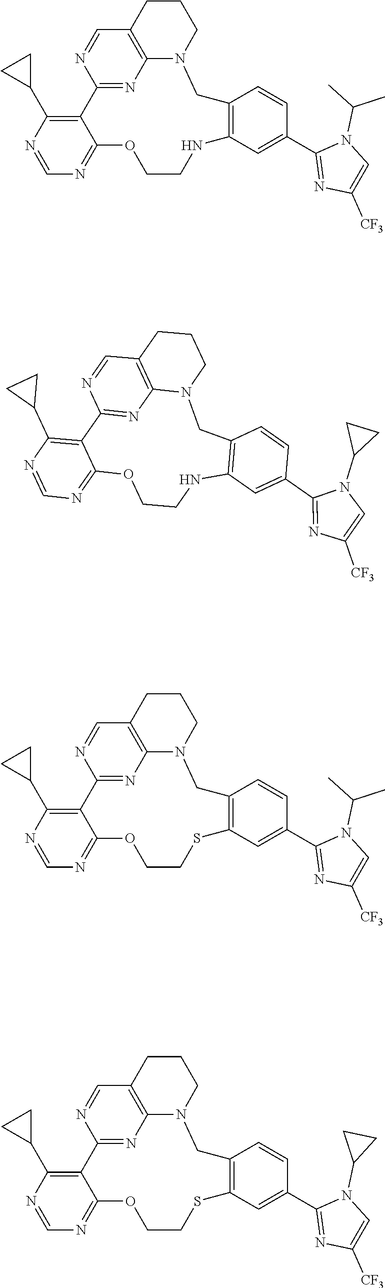 [SMILES: CC(C)n1cc(C(F)(F)F)nc1-c1ccc2c(c1)NCCOc1ncnc(C3CC3)c1-c1ncc3c(n1)N(CCC3)C2 ; FC(F)(F)c1cn(C2CC2)c(-c2ccc3c(c2)NCCOc2ncnc(C4CC4)c2-c2ncc4c(n2)N(CCC4)C3)n1 ; CC(C)n1cc(C(F)(F)F)nc1-c1ccc2c(c1)SCCOc1ncnc(C3CC3)c1-c1ncc3c(n1)N(CCC3)C2 ; FC(F)(F)c1cn(C2CC2)c(-c2ccc3c(c2)SCCOc2ncnc(C4CC4)c2-c2ncc4c(n2)N(CCC4)C3)n1]

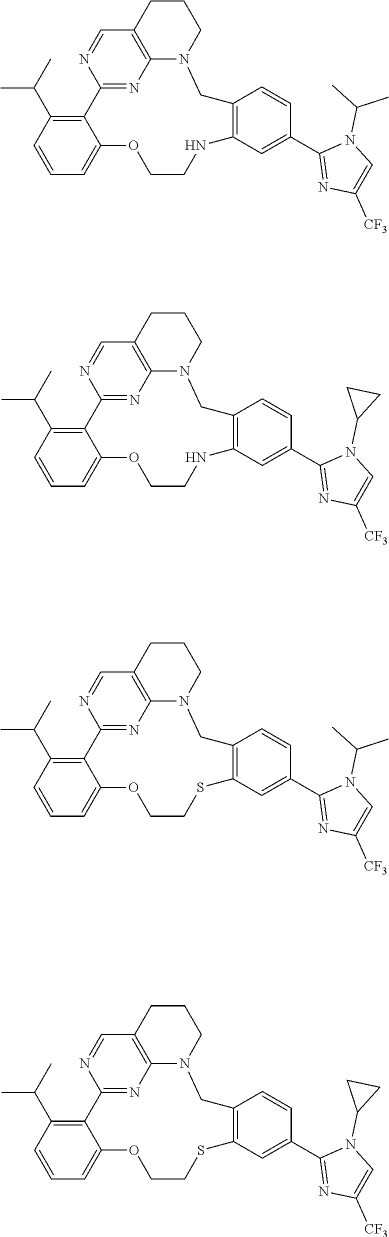

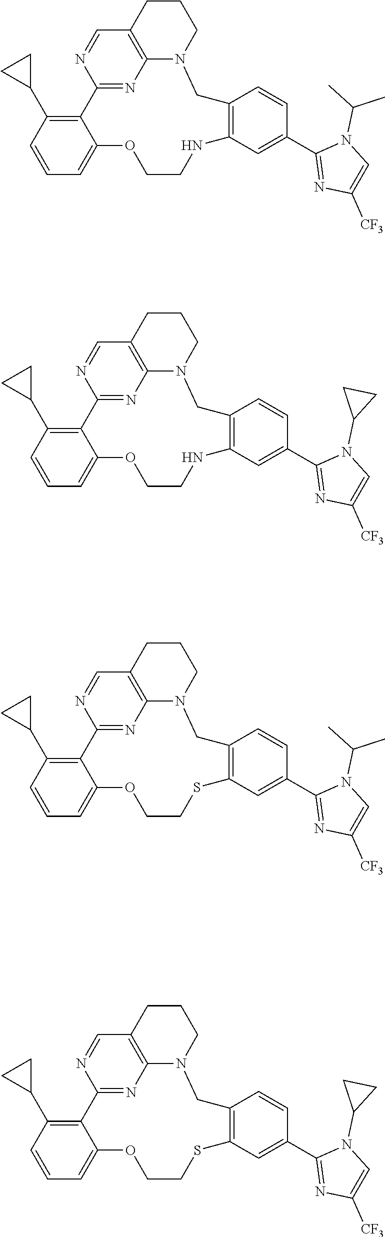 [SMILES: CC(C)n1cc(C(F)(F)F)nc1-c1ccc2c(c1)NCCOc1cccc(C3CC3)c1-c1ncc3c(n1)N(CCC3)C2 ; FC(F)(F)c1cn(C2CC2)c(-c2ccc3c(c2)NCCOc2cccc(C4CC4)c2-c2ncc4c(n2)N(CCC4)C3)n1 ; CC(C)n1cc(C(F)(F)F)nc1-c1ccc2c(c1)SCCOc1cccc(C3CC3)c1-c1ncc3c(n1)N(CCC3)C2 ; FC(F)(F)c1cn(C2CC2)c(-c2ccc3c(c2)SCCOc2cccc(C4CC4)c2-c2ncc4c(n2)N(CCC4)C3)n1]

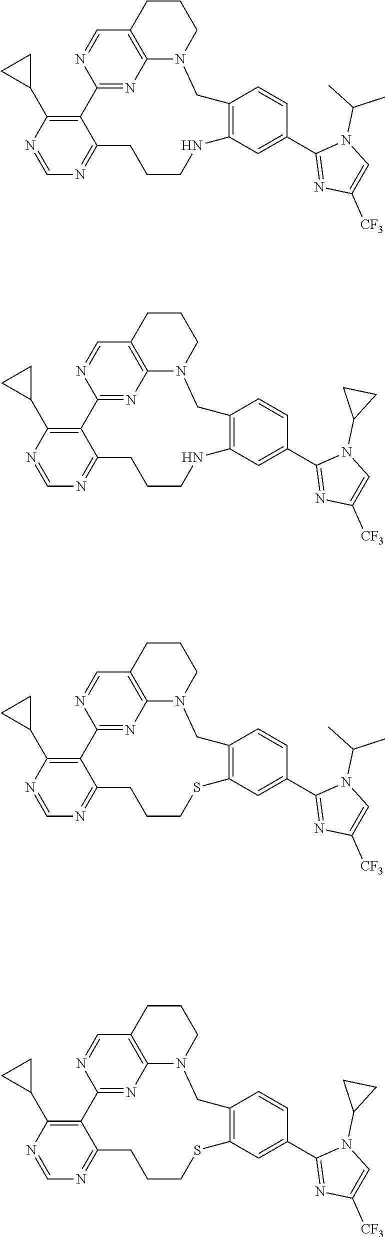 [SMILES: CC(C)n1cc(C(F)(F)F)nc1-c1ccc2c(c1)NCCCc1ncnc(C3CC3)c1-c1ncc3c(n1)N(CCC3)C2 ; FC(F)(F)c1cn(C2CC2)c(-c2ccc3c(c2)NCCCc2ncnc(C4CC4)c2-c2ncc4c(n2)N(CCC4)C3)n1 ; CC(C)n1cc(C(F)(F)F)nc1-c1ccc2c(c1)SCCCc1ncnc(C3CC3)c1-c1ncc3c(n1)N(CCC3)C2 ; FC(F)(F)c1cn(C2CC2)c(-c2ccc3c(c2)SCCCc2ncnc(C4CC4)c2-c2ncc4c(n2)N(CCC4)C3)n1]

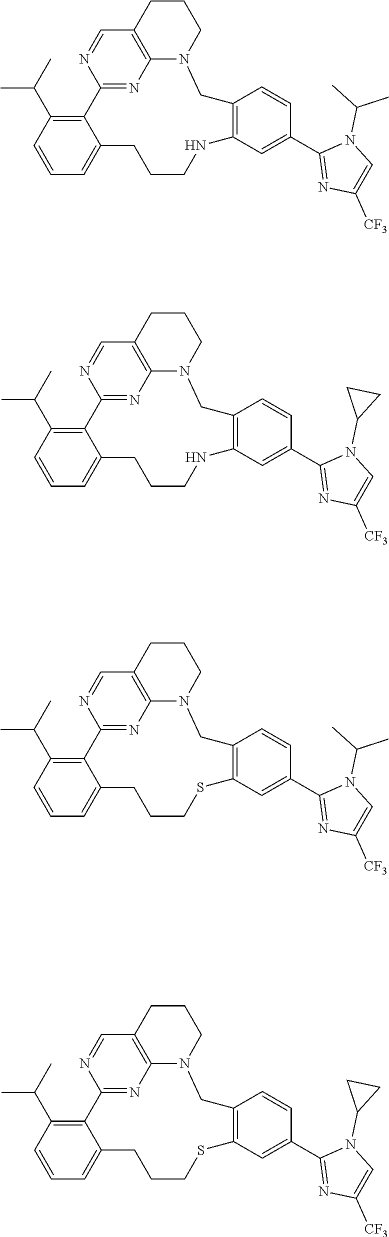

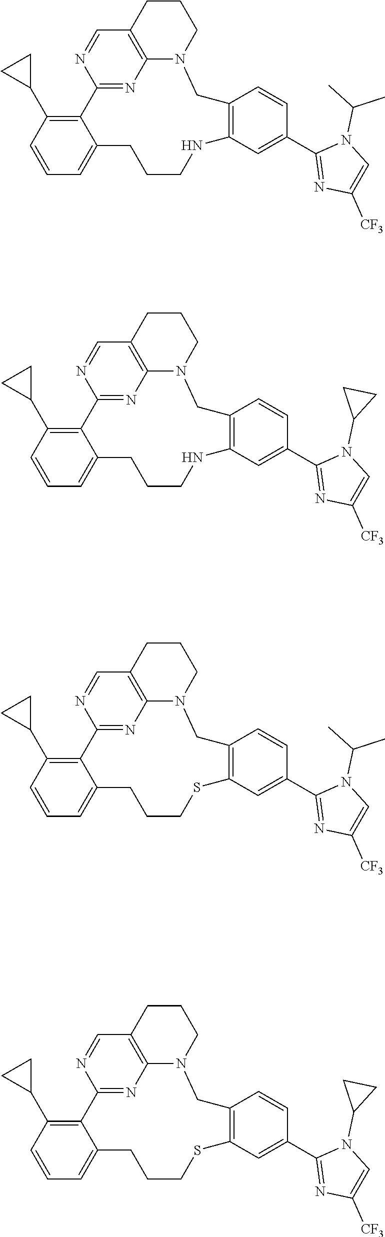 [SMILES: CC(C)n1cc(C(F)(F)F)nc1-c1ccc2c(c1)NCCCc1cccc(C3CC3)c1-c1ncc3c(n1)N(CCC3)C2 ; FC(F)(F)c1cn(C2CC2)c(-c2ccc3c(c2)NCCCc2cccc(C4CC4)c2-c2ncc4c(n2)N(CCC4)C3)n1 ; CC(C)n1cc(C(F)(F)F)nc1-c1ccc2c(c1)SCCCc1cccc(C3CC3)c1-c1ncc3c(n1)N(CCC3)C2 ; FC(F)(F)c1cn(C2CC2)c(-c2ccc3c(c2)SCCCc2cccc(C4CC4)c2-c2ncc4c(n2)N(CCC4)C3)n1]

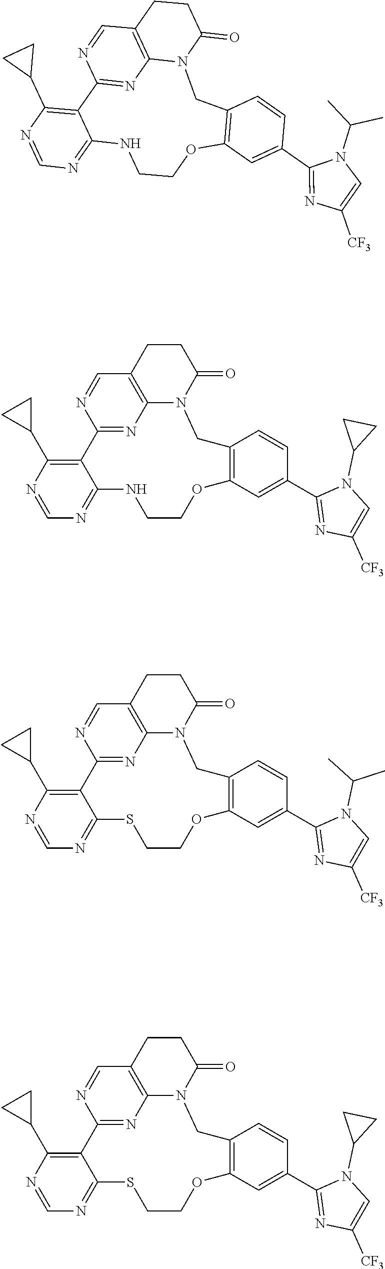 [SMILES: CC(C)n1cc(C(F)(F)F)nc1-c1ccc2c(c1)OCCNc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCNc1ncnc(C2CC2)c1-3 ; CC(C)n1cc(C(F)(F)F)nc1-c1ccc2c(c1)OCCSc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCSc1ncnc(C2CC2)c1-3]

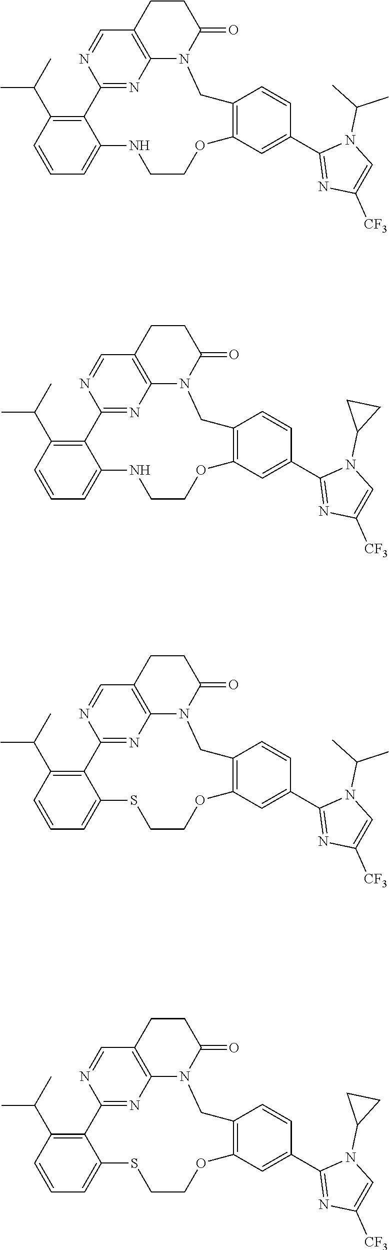

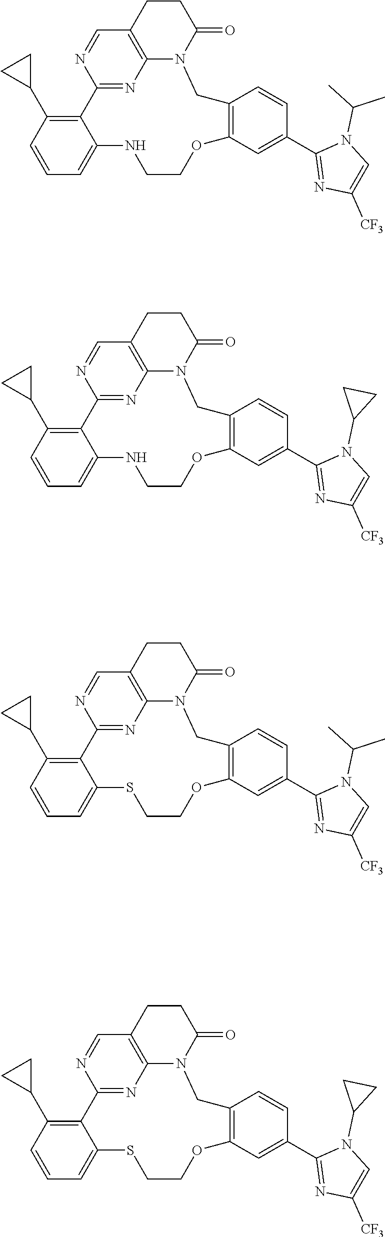 [SMILES: CC(C)n1cc(C(F)(F)F)nc1-c1ccc2c(c1)OCCNc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCNc1cccc(C2CC2)c1-3 ; CC(C)n1cc(C(F)(F)F)nc1-c1ccc2c(c1)OCCSc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1OCCSc1cccc(C2CC2)c1-3]

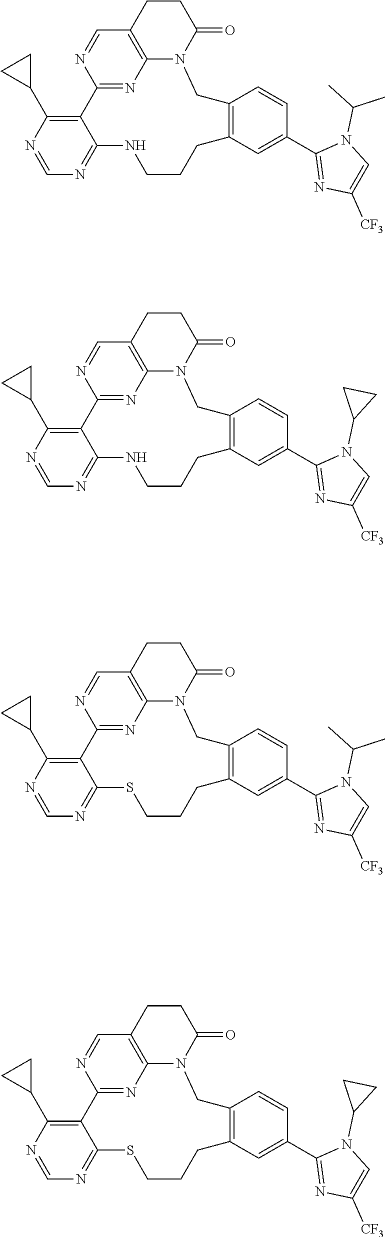 [SMILES: CC(C)n1cc(C(F)(F)F)nc1-c1ccc2c(c1)CCCNc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCNc1ncnc(C2CC2)c1-3 ; CC(C)n1cc(C(F)(F)F)nc1-c1ccc2c(c1)CCCSc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCSc1ncnc(C2CC2)c1-3]

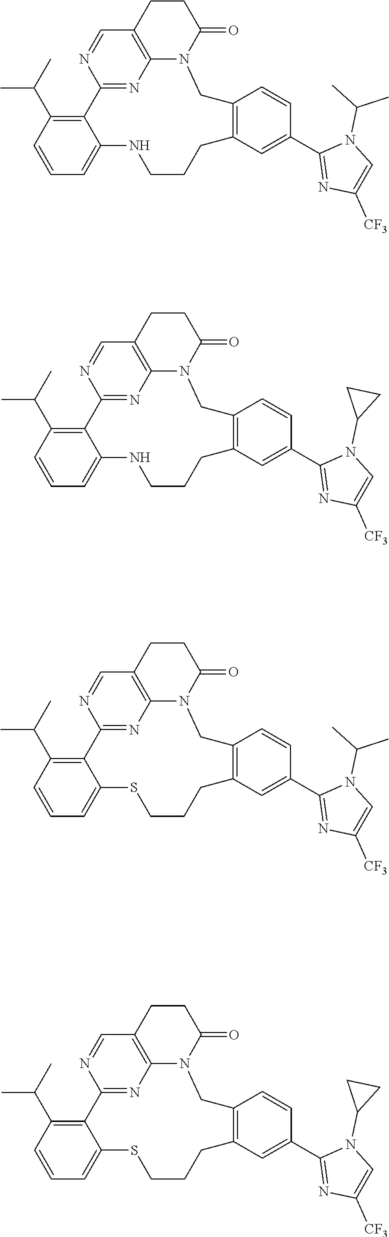

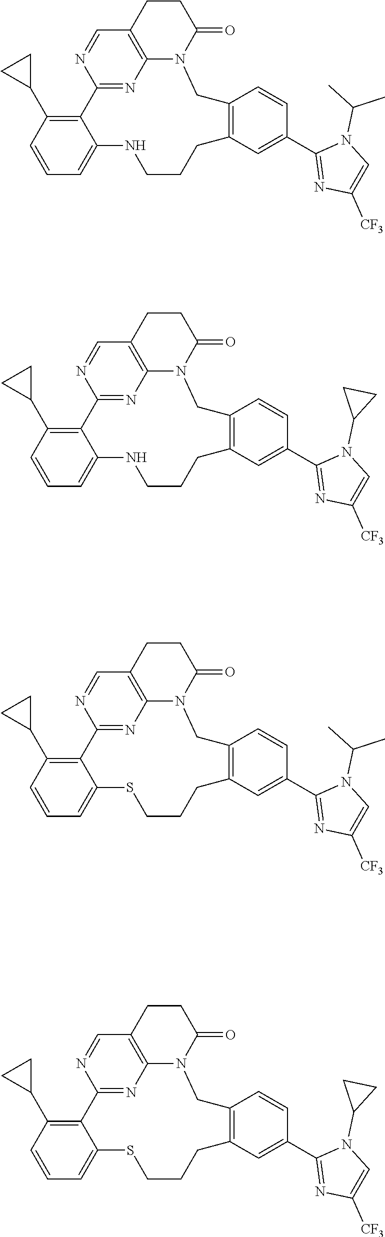 [SMILES: CC(C)n1cc(C(F)(F)F)nc1-c1ccc2c(c1)CCCNc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCNc1cccc(C2CC2)c1-3 ; CC(C)n1cc(C(F)(F)F)nc1-c1ccc2c(c1)CCCSc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1CCCSc1cccc(C2CC2)c1-3]

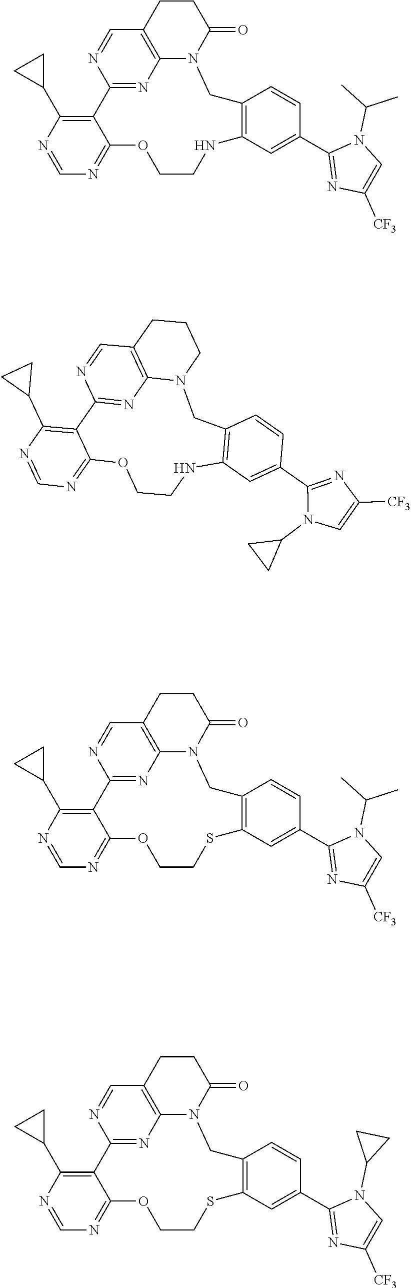 [SMILES: CC(C)n1cc(C(F)(F)F)nc1-c1ccc2c(c1)NCCOc1ncnc(C3CC3)c1-c1ncc3c(n1)N(C2)C(=O)CC3 ; FC(F)(F)c1cn(C2CC2)c(-c2ccc3c(c2)NCCOc2ncnc(C4CC4)c2-c2ncc4c(n2)N(CCC4)C3)n1 ; CC(C)n1cc(C(F)(F)F)nc1-c1ccc2c(c1)SCCOc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1SCCOc1ncnc(C2CC2)c1-3]

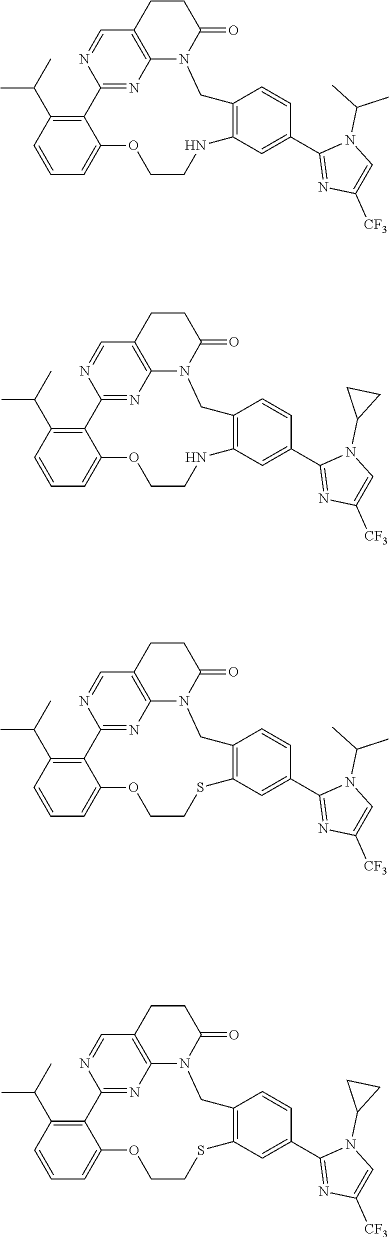

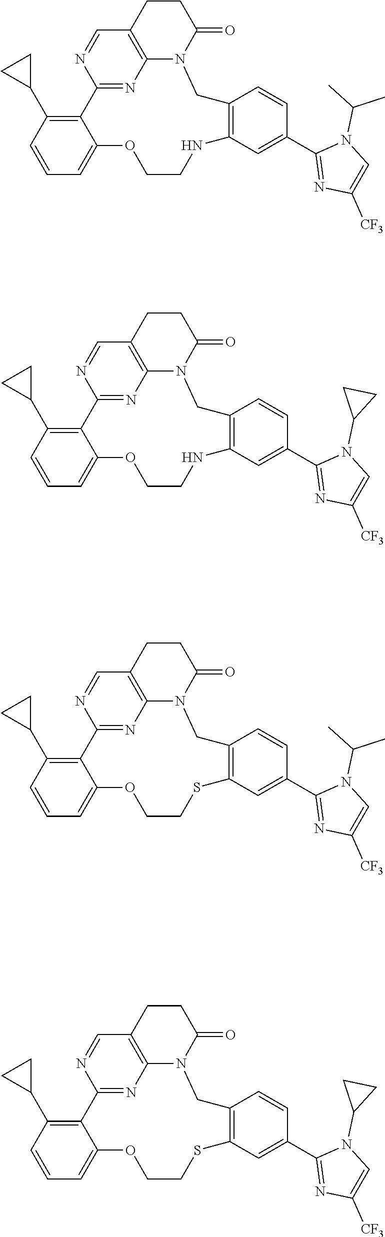 [SMILES: CC(C)n1cc(C(F)(F)F)nc1-c1ccc2c(c1)NCCOc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1NCCOc1cccc(C2CC2)c1-3 ; CC(C)n1cc(C(F)(F)F)nc1-c1ccc2c(c1)SCCOc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1SCCOc1cccc(C2CC2)c1-3]

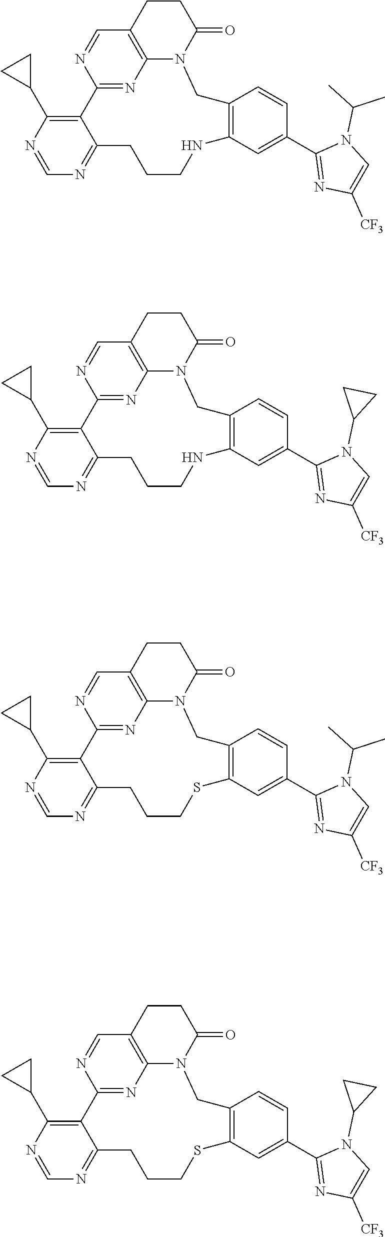 [SMILES: CC(C)n1cc(C(F)(F)F)nc1-c1ccc2c(c1)NCCCc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1NCCCc1ncnc(C2CC2)c1-3 ; CC(C)n1cc(C(F)(F)F)nc1-c1ccc2c(c1)SCCCc1ncnc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1SCCCc1ncnc(C2CC2)c1-3]

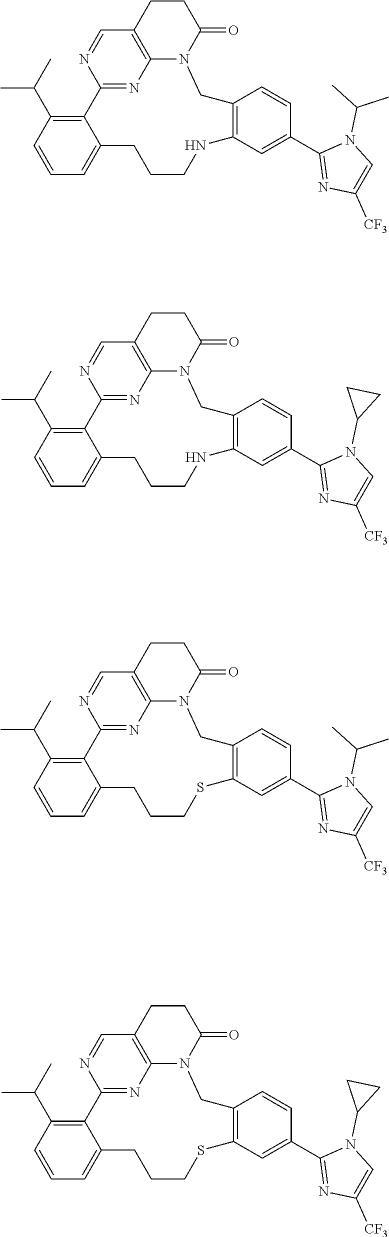

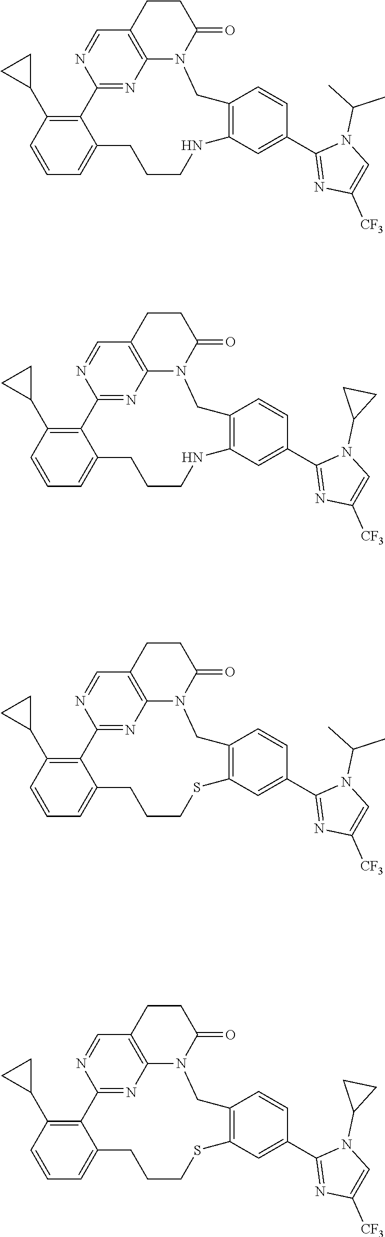 [SMILES: CC(C)n1cc(C(F)(F)F)nc1-c1ccc2c(c1)NCCCc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1NCCCc1cccc(C2CC2)c1-3 ; CC(C)n1cc(C(F)(F)F)nc1-c1ccc2c(c1)SCCCc1cccc(C3CC3)c1-c1ncc3c(n1)N(C2)C(=O)CC3 ; O=C1CCc2cnc3nc2N1Cc1ccc(-c2nc(C(F)(F)F)cn2C2CC2)cc1SCCCc1cccc(C2CC2)c1-3]

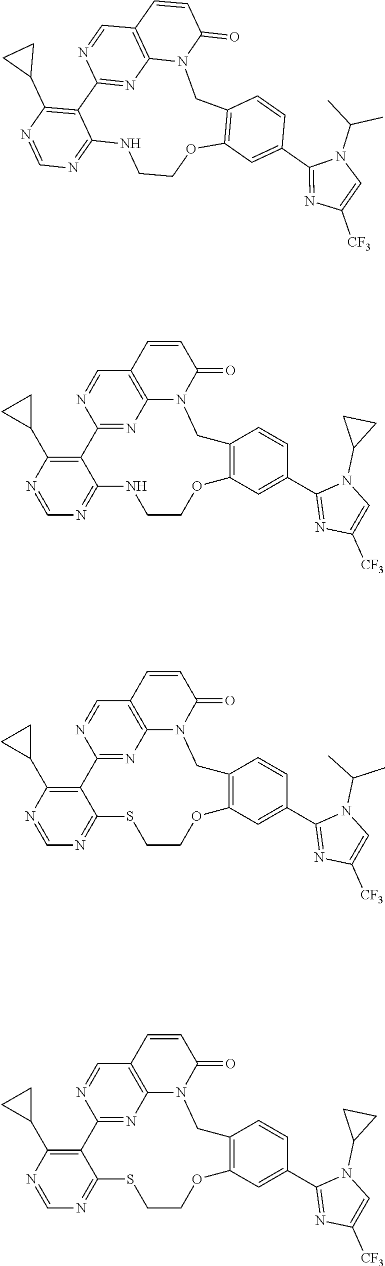 [SMILES: CC(C)n1cc(C(F)(F)F)nc1-c1ccc2c(c1)OCCNc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCNc1ncnc(C2CC2)c1-3 ; CC(C)n1cc(C(F)(F)F)nc1-c1ccc2c(c1)OCCSc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCSc1ncnc(C2CC2)c1-3]

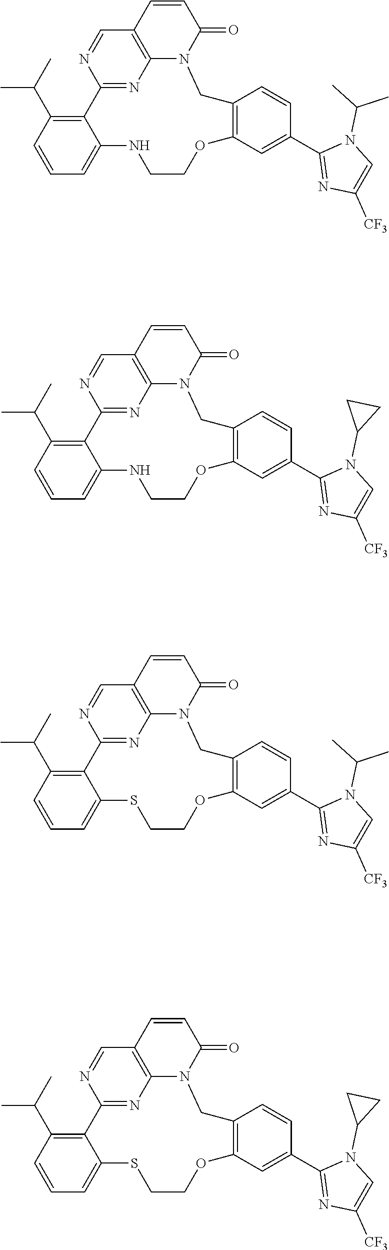

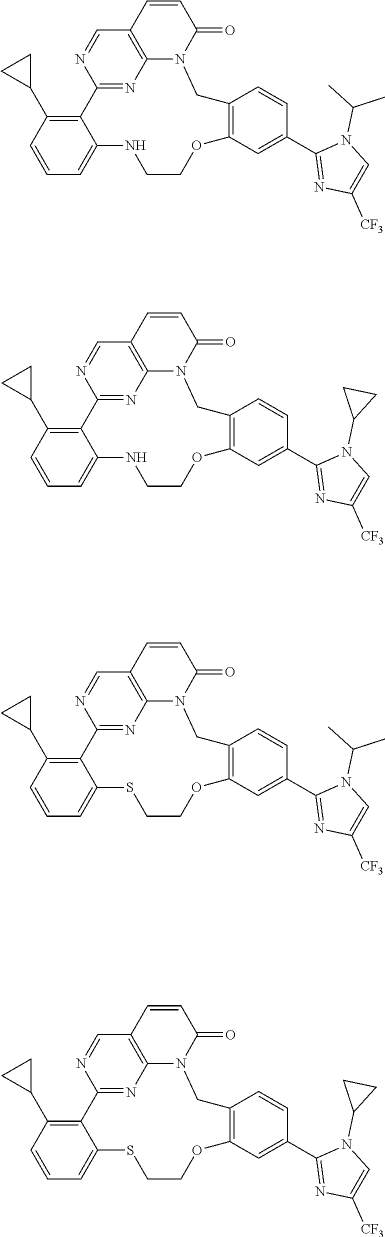 [SMILES: CC(C)n1cc(C(F)(F)F)nc1-c1ccc2c(c1)OCCNc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCNc1cccc(C2CC2)c1-3 ; CC(C)n1cc(C(F)(F)F)nc1-c1ccc2c(c1)OCCSc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1OCCSc1cccc(C2CC2)c1-3]

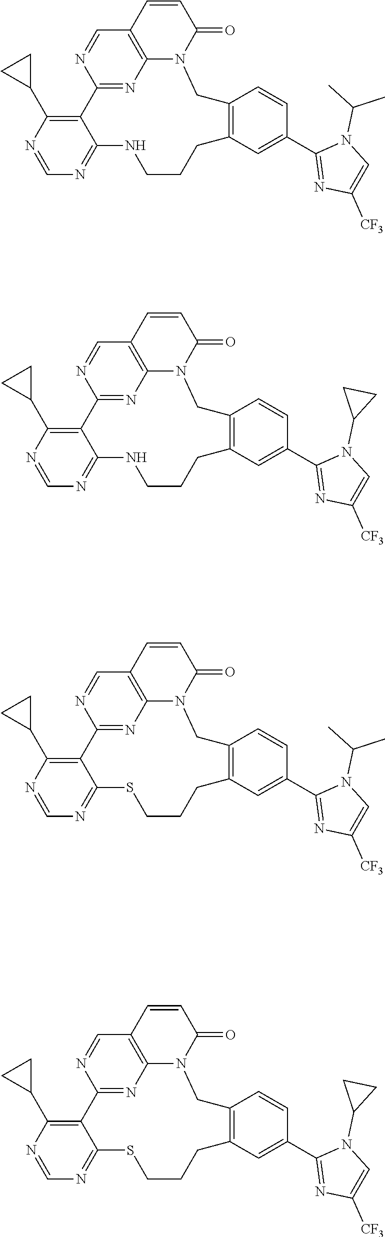 [SMILES: CC(C)n1cc(C(F)(F)F)nc1-c1ccc2c(c1)CCCNc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCNc1ncnc(C2CC2)c1-3 ; CC(C)n1cc(C(F)(F)F)nc1-c1ccc2c(c1)CCCSc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCSc1ncnc(C2CC2)c1-3]

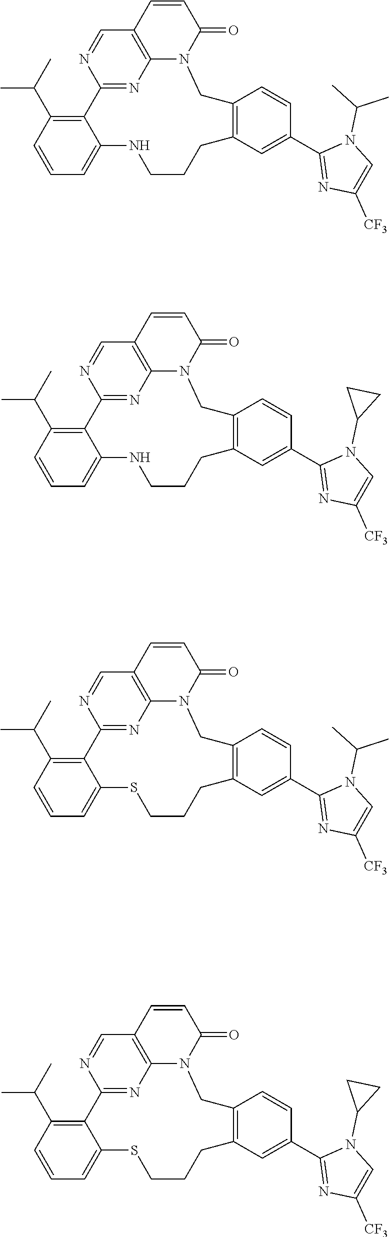

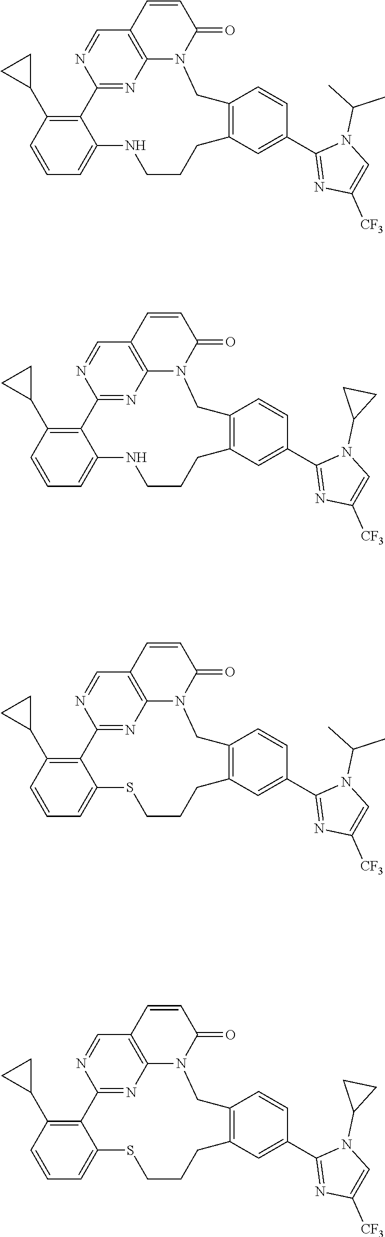 [SMILES: CC(C)n1cc(C(F)(F)F)nc1-c1ccc2c(c1)CCCNc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCNc1cccc(C2CC2)c1-3 ; CC(C)n1cc(C(F)(F)F)nc1-c1ccc2c(c1)CCCSc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1CCCSc1cccc(C2CC2)c1-3]

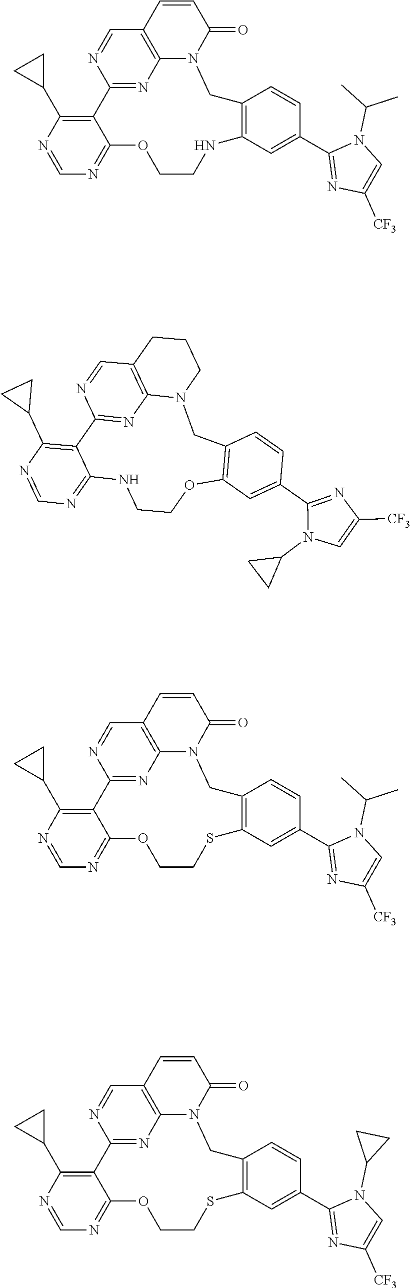 [SMILES: CC(C)n1cc(C(F)(F)F)nc1-c1ccc2c(c1)NCCOc1ncnc(C3CC3)c1-c1ncc3ccc(=O)n(c3n1)C2 ; FC(F)(F)c1cn(C2CC2)c(-c2ccc3c(c2)OCCNc2ncnc(C4CC4)c2-c2ncc4c(n2)N(CCC4)C3)n1 ; CC(C)n1cc(C(F)(F)F)nc1-c1ccc2c(c1)SCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1SCCOc1ncnc(C2CC2)c1-3]

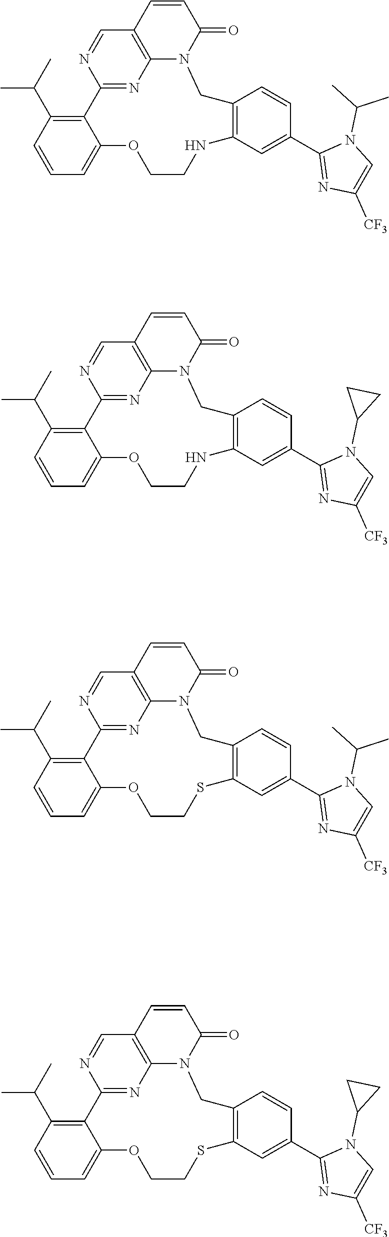

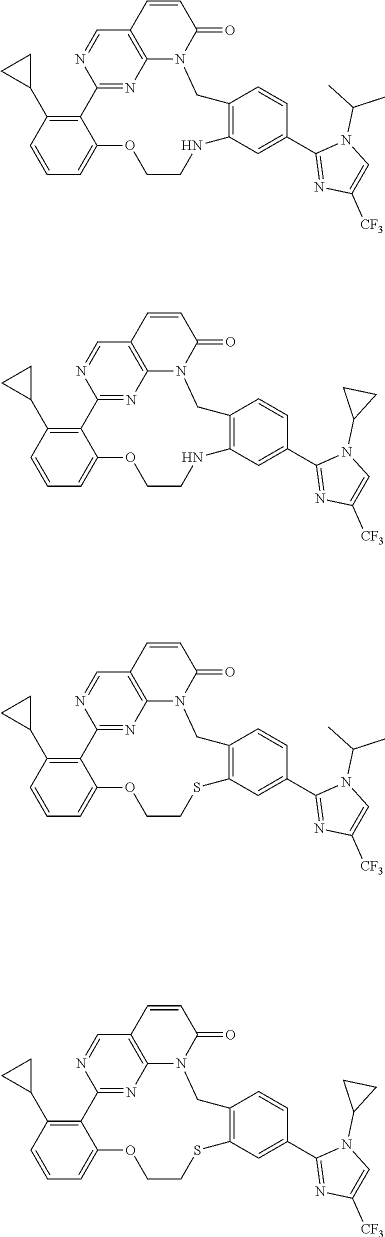 [SMILES: CC(C)n1cc(C(F)(F)F)nc1-c1ccc2c(c1)NCCOc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1NCCOc1cccc(C2CC2)c1-3 ; CC(C)n1cc(C(F)(F)F)nc1-c1ccc2c(c1)SCCOc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1SCCOc1cccc(C2CC2)c1-3]

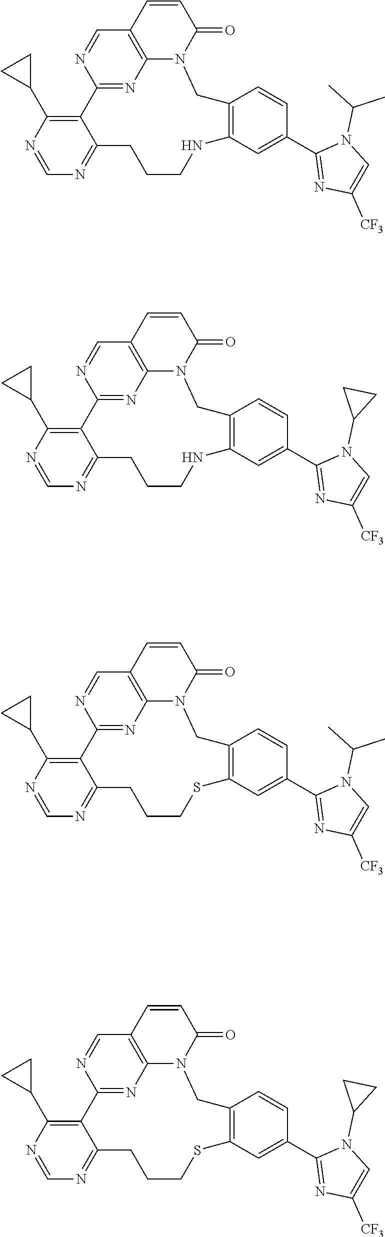 [SMILES: CC(C)n1cc(C(F)(F)F)nc1-c1ccc2c(c1)NCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1NCCCc1ncnc(C2CC2)c1-3 ; CC(C)n1cc(C(F)(F)F)nc1-c1ccc2c(c1)SCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1SCCCc1ncnc(C2CC2)c1-3]

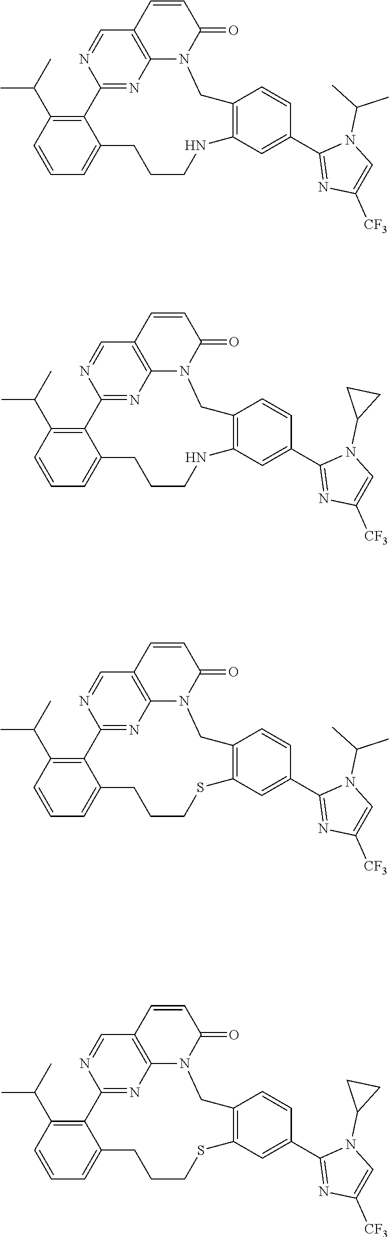

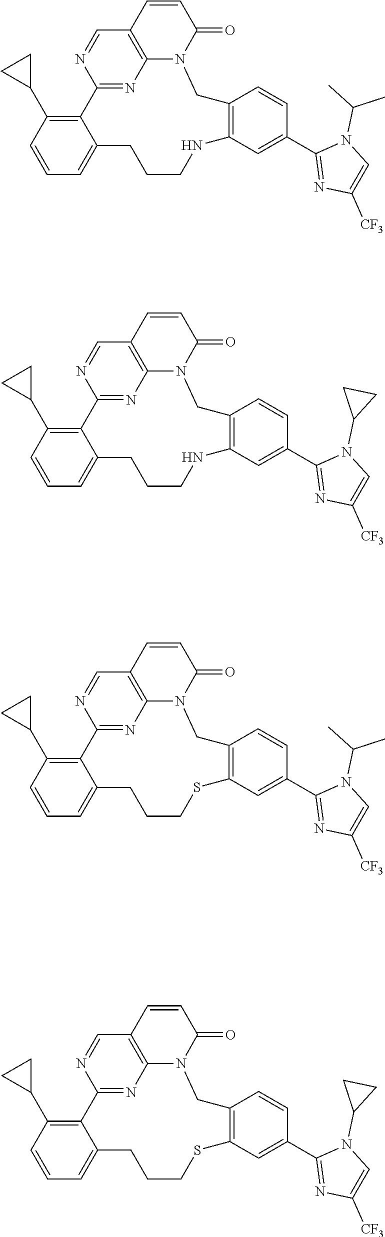 [SMILES: CC(C)n1cc(C(F)(F)F)nc1-c1ccc2c(c1)NCCCc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1NCCCc1cccc(C2CC2)c1-3 ; CC(C)n1cc(C(F)(F)F)nc1-c1ccc2c(c1)SCCCc1cccc(C3CC3)c1-c1ncc3ccc(=O)n(c3n1)C2 ; O=c1ccc2cnc3nc2n1Cc1ccc(-c2nc(C(F)(F)F)cn2C2CC2)cc1SCCCc1cccc(C2CC2)c1-3]

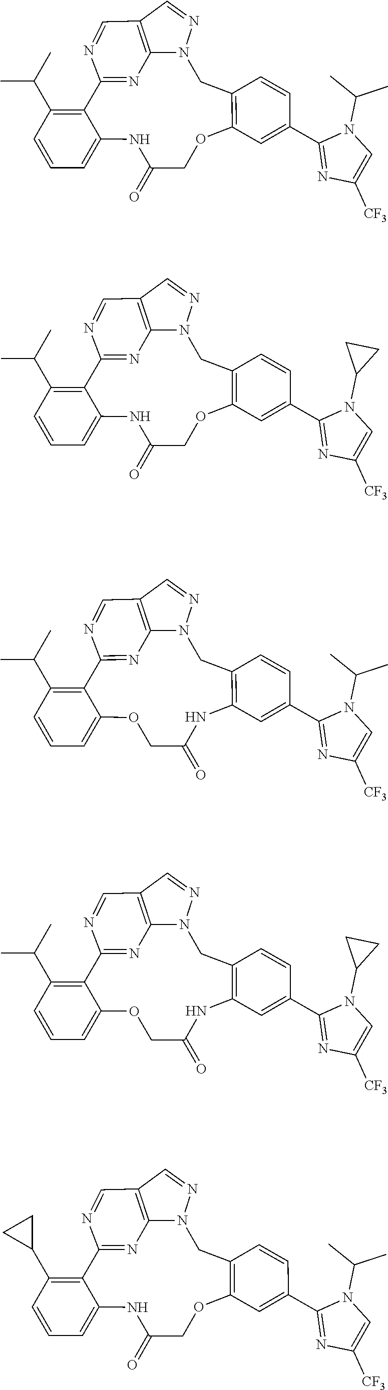

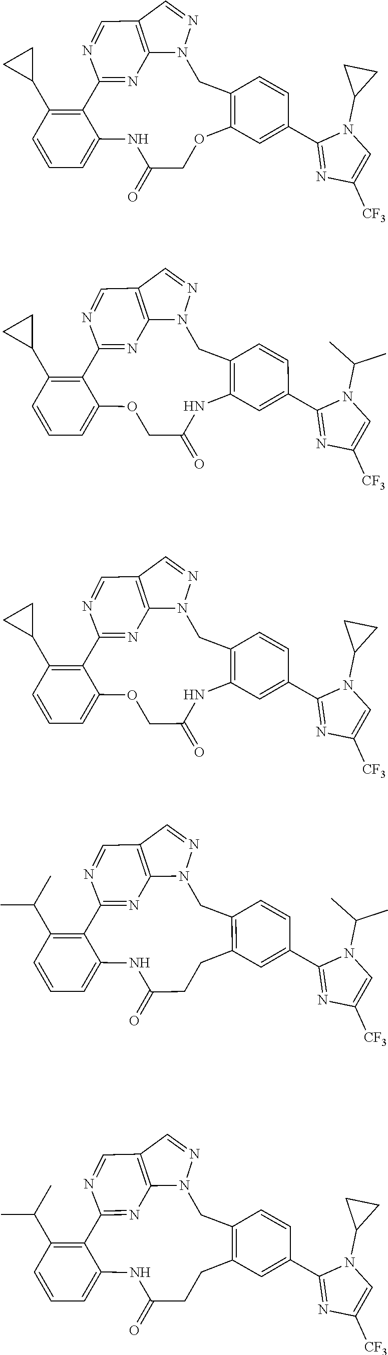

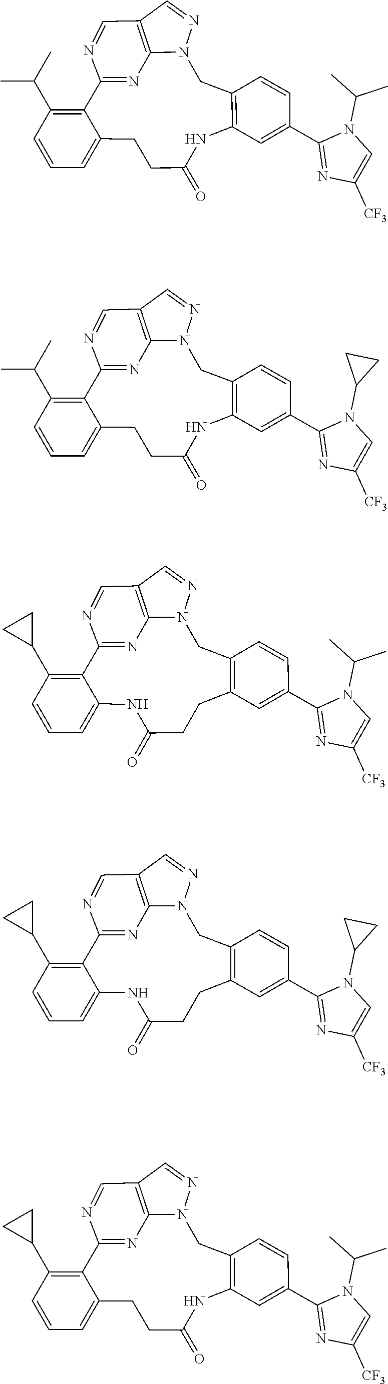

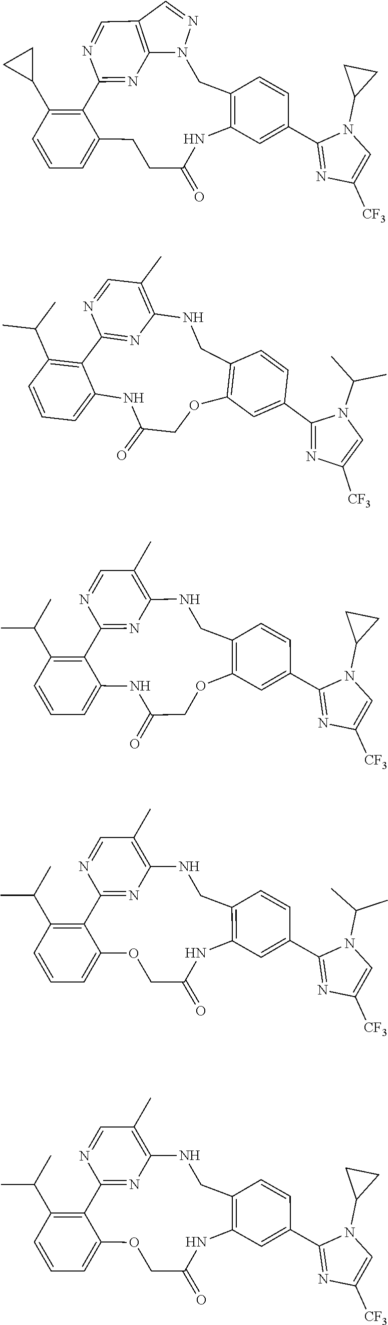

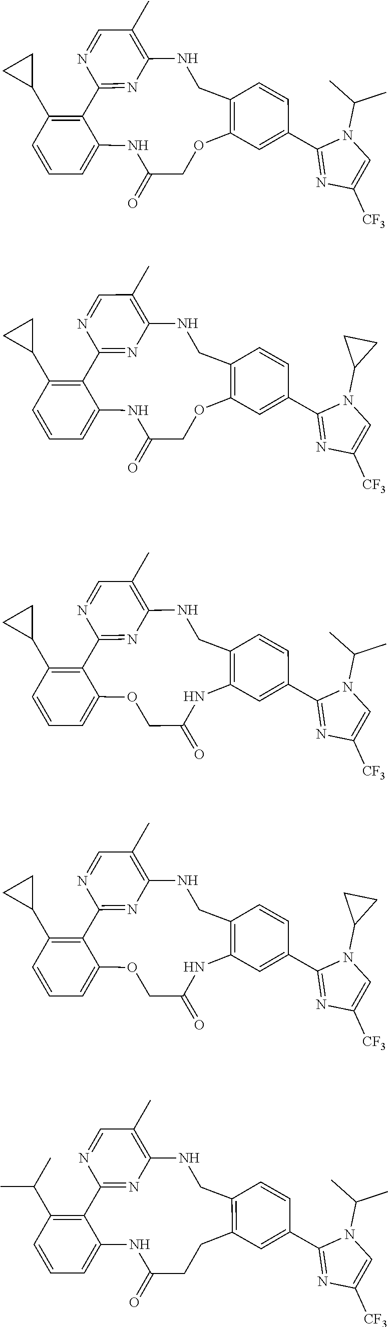

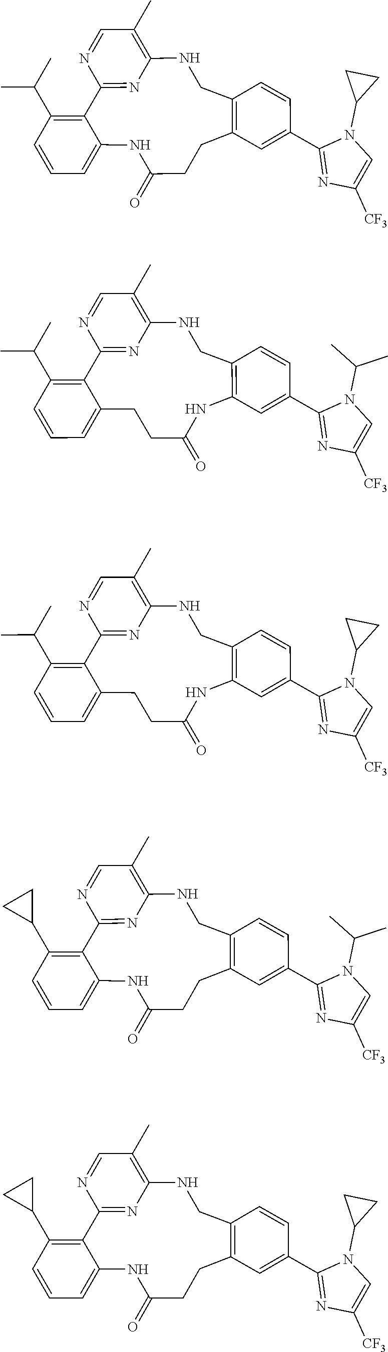

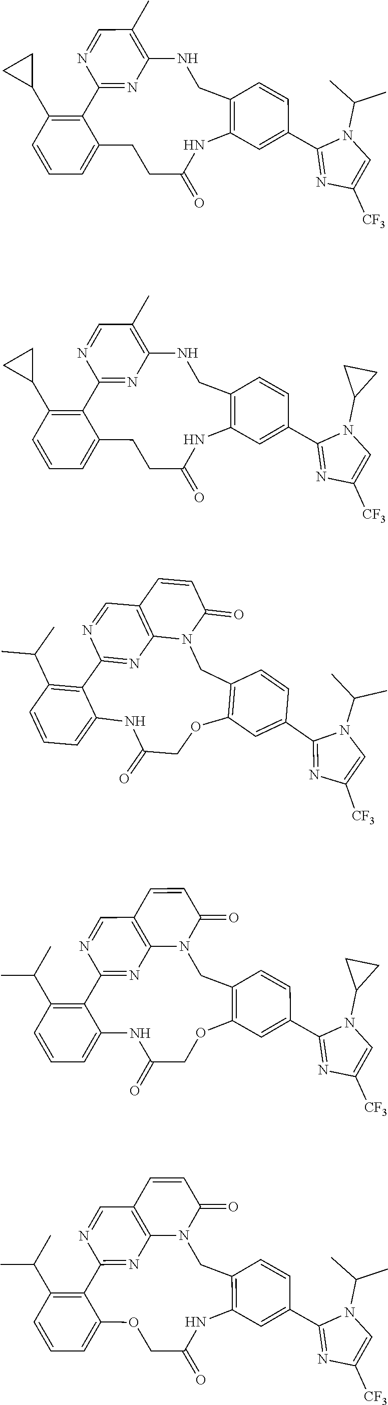

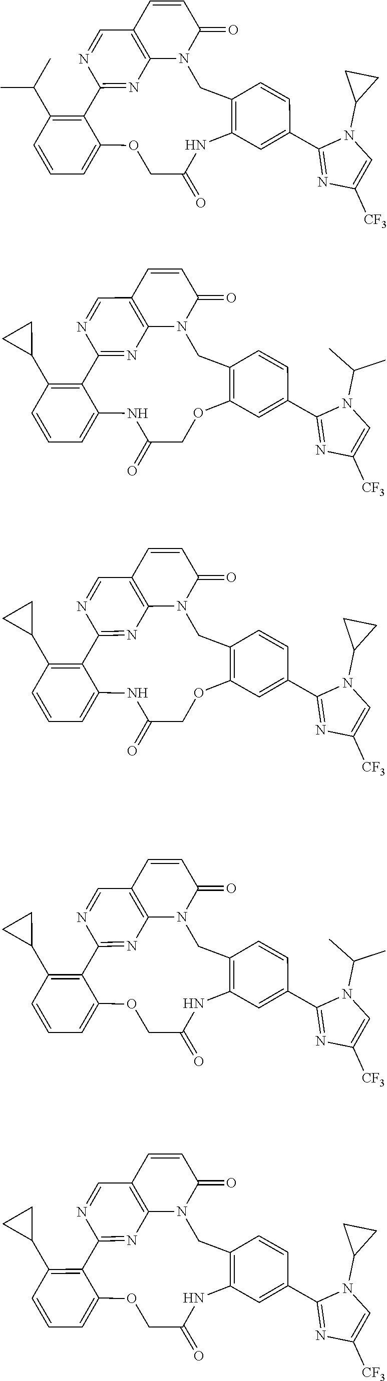 [SMILES: CC(C)c1cccc2c1-c1ncc3ccc(=O)n(c3n1)Cc1ccc(-c3nc(C(F)(F)F)cn3C3CC3)cc1NC(=O)CO2 ; CC(C)n1cc(C(F)(F)F)nc1-c1ccc2c(c1)OCC(=O)Nc1cccc(C3CC3)c1-c1ncc3ccc(=O)n(c3n1)C2 ; O=C1COc2cc(-c3nc(C(F)(F)F)cn3C3CC3)ccc2Cn2c(=O)ccc3cnc(nc32)-c2c(cccc2C2CC2)N1 ; CC(C)n1cc(C(F)(F)F)nc1-c1ccc2c(c1)NC(=O)COc1cccc(C3CC3)c1-c1ncc3ccc(=O)n(c3n1)C2 ; O=C1COc2cccc(C3CC3)c2-c2ncc3ccc(=O)n(c3n2)Cc2ccc(-c3nc(C(F)(F)F)cn3C3CC3)cc2N1]

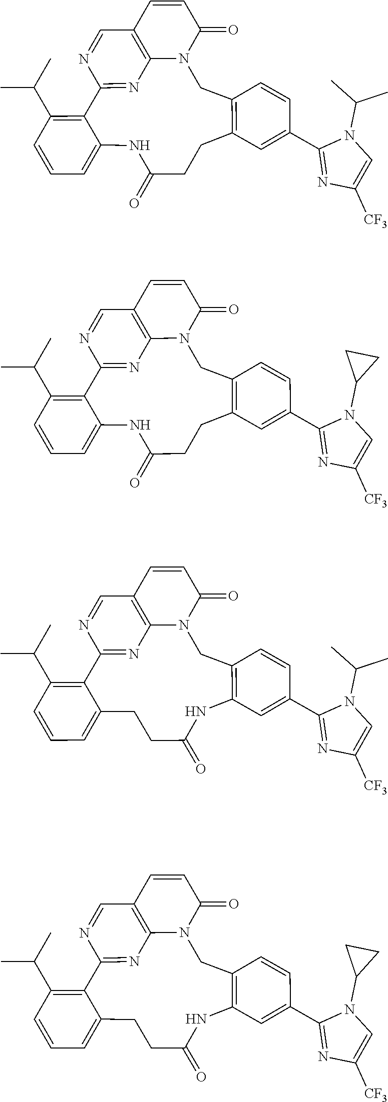

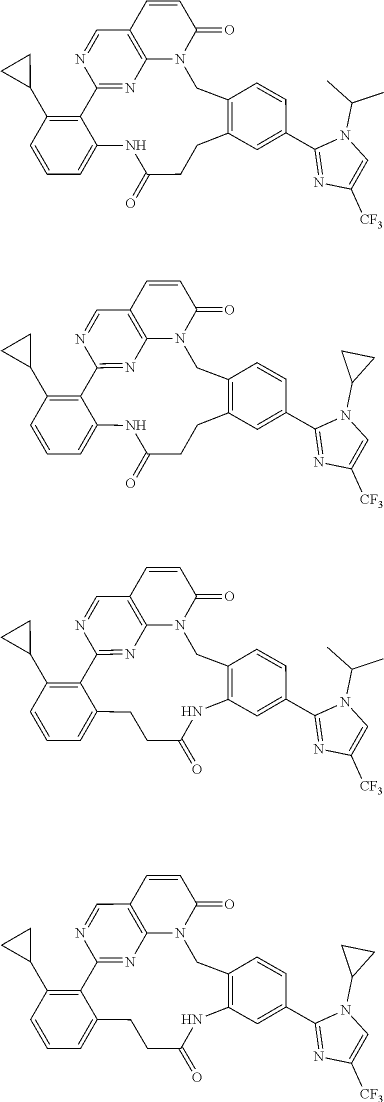 [SMILES: CC(C)n1cc(C(F)(F)F)nc1-c1ccc2c(c1)CCC(=O)Nc1cccc(C3CC3)c1-c1ncc3ccc(=O)n(c3n1)C2 ; O=C1CCc2cc(-c3nc(C(F)(F)F)cn3C3CC3)ccc2Cn2c(=O)ccc3cnc(nc32)-c2c(cccc2C2CC2)N1 ; CC(C)n1cc(C(F)(F)F)nc1-c1ccc2c(c1)NC(=O)CCc1cccc(C3CC3)c1-c1ncc3ccc(=O)n(c3n1)C2 ; O=C1CCc2cccc(C3CC3)c2-c2ncc3ccc(=O)n(c3n2)Cc2ccc(-c3nc(C(F)(F)F)cn3C3CC3)cc2N1]

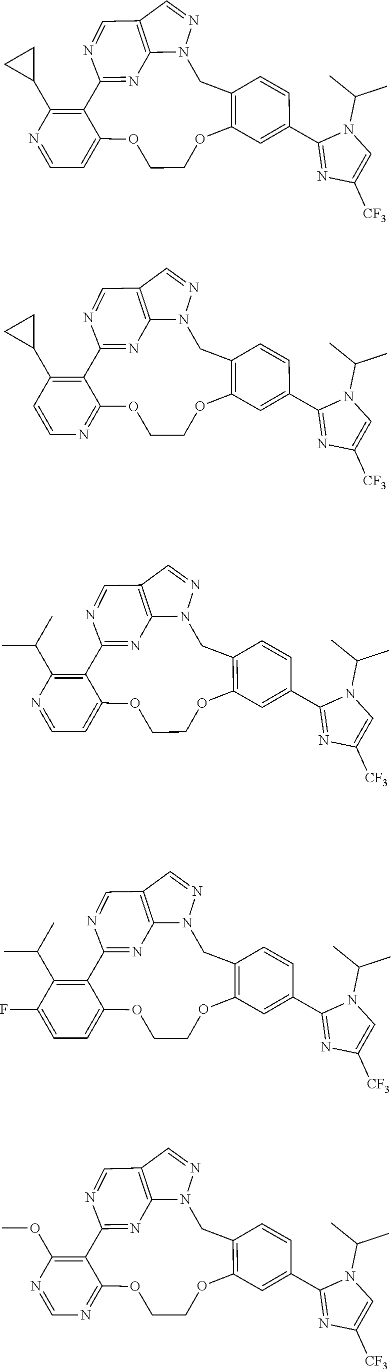

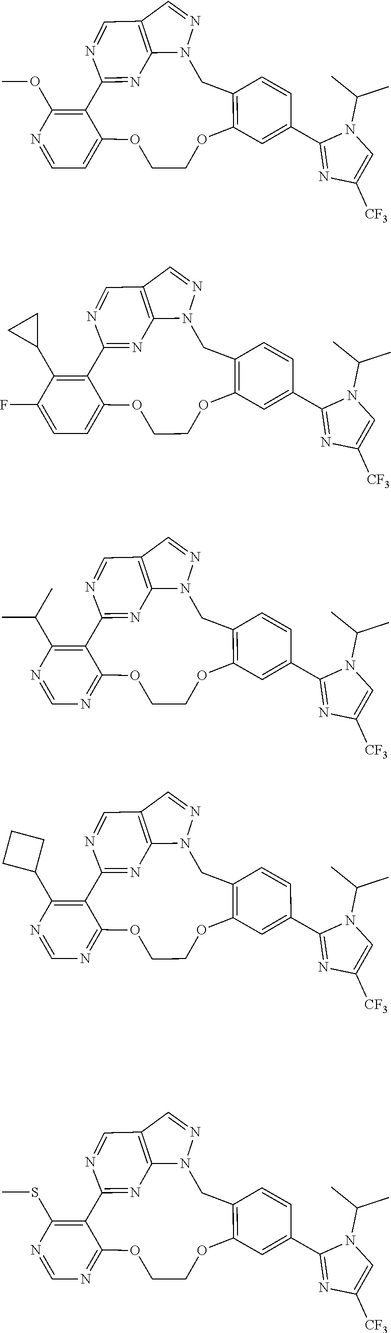

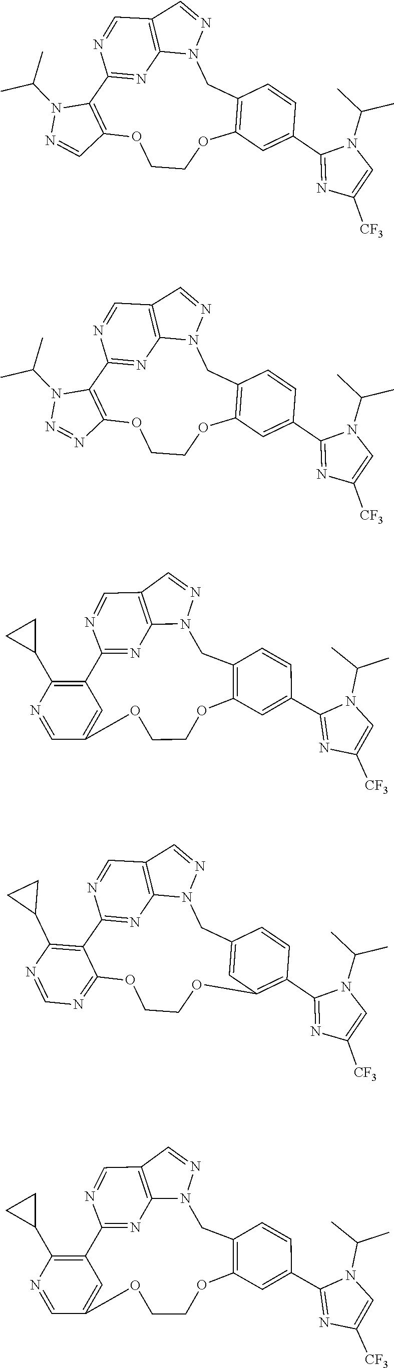

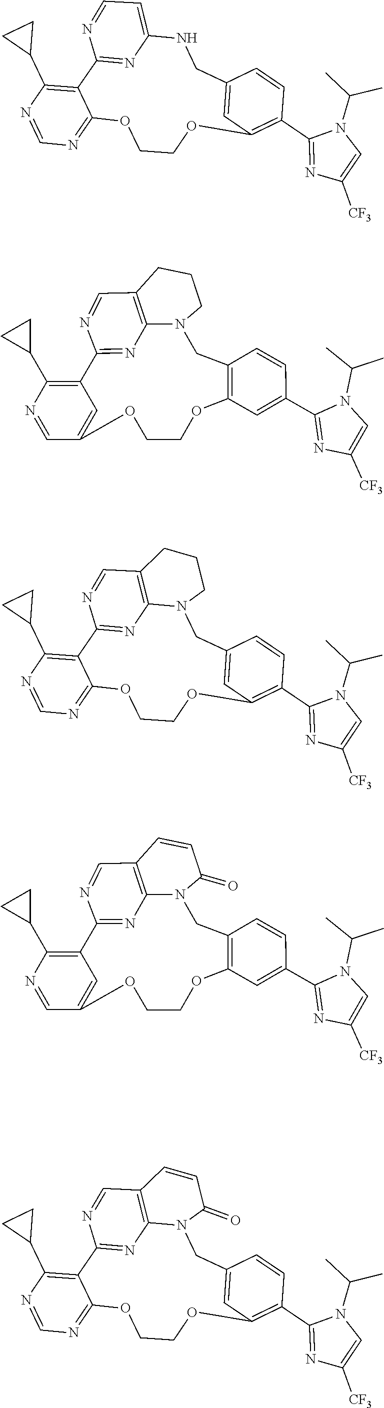 [SMILES: CC(C)n1cc(C(F)(F)F)nc1-c1ccc2cc1OCCOc1ncnc(C3CC3)c1-c1nccc(n1)NC2 ; CC(C)n1cc(C(F)(F)F)nc1-c1ccc2c(c1)OCCOc1cnc(C3CC3)c(c1)-c1ncc3c(n1)N(CCC3)C2 ; CC(C)n1cc(C(F)(F)F)nc1-c1ccc2cc1OCCOc1ncnc(C3CC3)c1-c1ncc3c(n1)N(CCC3)C2 ; CC(C)n1cc(C(F)(F)F)nc1-c1ccc2c(c1)OCCOc1cnc(C3CC3)c(c1)-c1ncc3ccc(=O)n(c3n1)C2 ; CC(C)n1cc(C(F)(F)F)nc1-c1ccc2cc1OCCOc1ncnc(C3CC3)c1-c1ncc3ccc(=O)n(c3n1)C2]

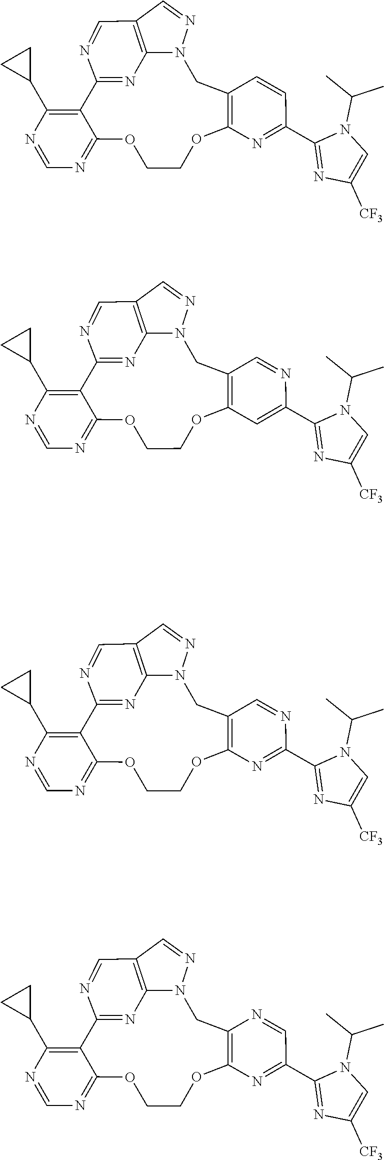 [SMILES: CC(C)n1cc(C(F)(F)F)nc1-c1ccc2c(n1)OCCOc1ncnc(C3CC3)c1-c1ncc3cnn(c3n1)C2 ; CC(C)n1cc(C(F)(F)F)nc1-c1cc2c(cn1)Cn1ncc3cnc(nc31)-c1c(ncnc1C1CC1)OCCO2 ; CC(C)n1cc(C(F)(F)F)nc1-c1ncc2c(n1)OCCOc1ncnc(C3CC3)c1-c1ncc3cnn(c3n1)C2 ; CC(C)n1cc(C(F)(F)F)nc1-c1cnc2c(n1)OCCOc1ncnc(C3CC3)c1-c1ncc3cnn(c3n1)C2]

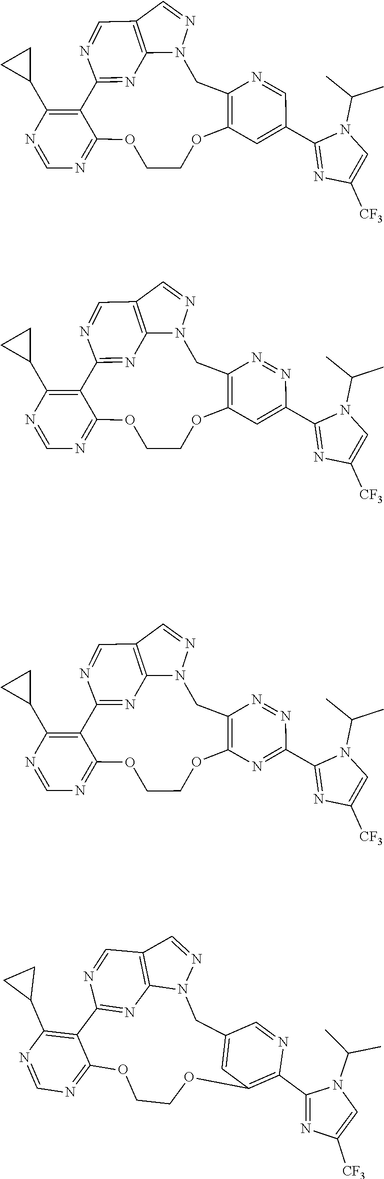 [SMILES: CC(C)n1cc(C(F)(F)F)nc1-c1cnc2c(c1)OCCOc1ncnc(C3CC3)c1-c1ncc3cnn(c3n1)C2 ; CC(C)n1cc(C(F)(F)F)nc1-c1cc2c(nn1)Cn1ncc3cnc(nc31)-c1c(ncnc1C1CC1)OCCO2 ; CC(C)n1cc(C(F)(F)F)nc1-c1nnc2c(n1)OCCOc1ncnc(C3CC3)c1-c1ncc3cnn(c3n1)C2 ; CC(C)n1cc(C(F)(F)F)nc1-c1ncc2cc1OCCOc1ncnc(C3CC3)c1-c1ncc3cnn(c3n1)C2]

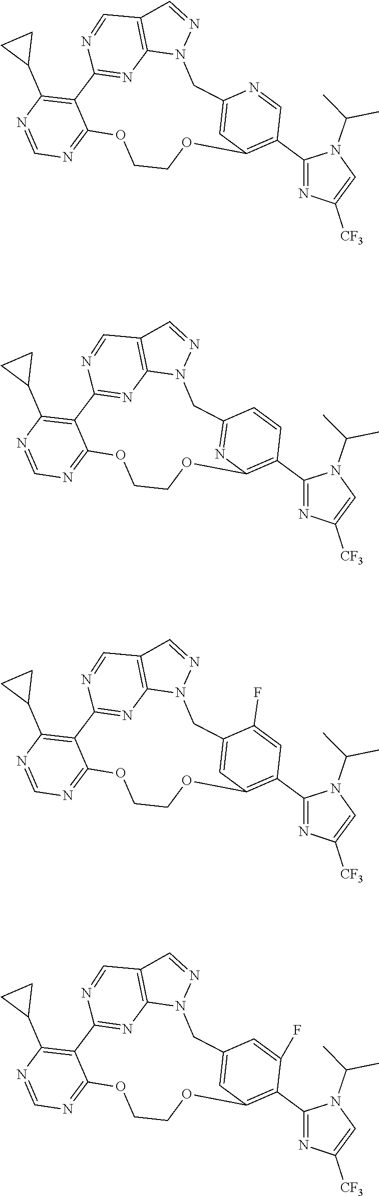 [SMILES: CC(C)n1cc(C(F)(F)F)nc1-c1cnc2cc1OCCOc1ncnc(C3CC3)c1-c1ncc3cnn(c3n1)C2 ; CC(C)n1cc(C(F)(F)F)nc1-c1ccc2nc1OCCOc1ncnc(C3CC3)c1-c1ncc3cnn(c3n1)C2 ; CC(C)n1cc(C(F)(F)F)nc1-c1cc(F)c2cc1OCCOc1ncnc(C3CC3)c1-c1ncc3cnn(c3n1)C2 ; CC(C)n1cc(C(F)(F)F)nc1-c1c(F)cc2cc1OCCOc1ncnc(C3CC3)c1-c1ncc3cnn(c3n1)C2]

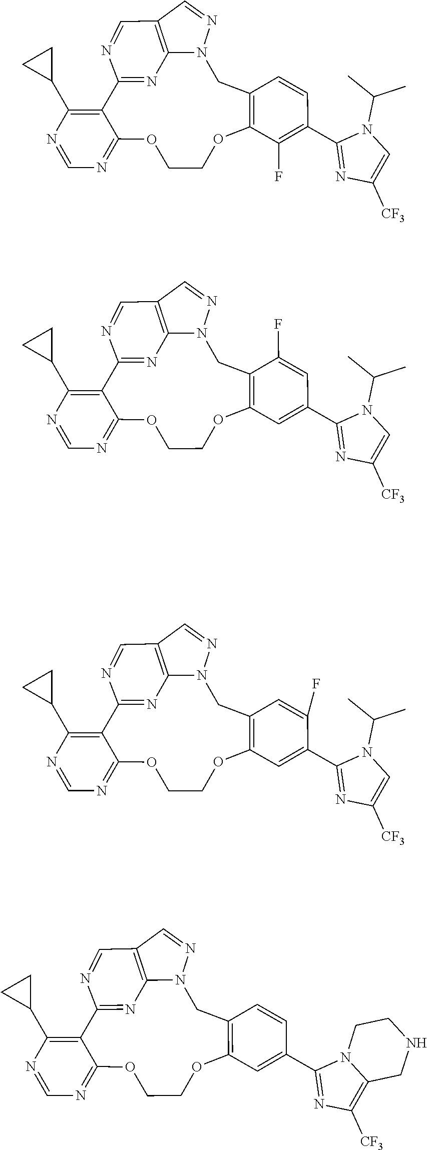 [SMILES: CC(C)n1cc(C(F)(F)F)nc1-c1ccc2c(c1F)OCCOc1ncnc(C3CC3)c1-c1ncc3cnn(c3n1)C2 ; CC(C)n1cc(C(F)(F)F)nc1-c1cc(F)c2c(c1)OCCOc1ncnc(C3CC3)c1-c1ncc3cnn(c3n1)C2 ; CC(C)n1cc(C(F)(F)F)nc1-c1cc2c(cc1F)Cn1ncc3cnc(nc31)-c1c(ncnc1C1CC1)OCCO2 ; FC(F)(F)c1nc(-c2ccc3c(c2)OCCOc2ncnc(C4CC4)c2-c2ncc4cnn(c4n2)C3)n2c1CNCC2]

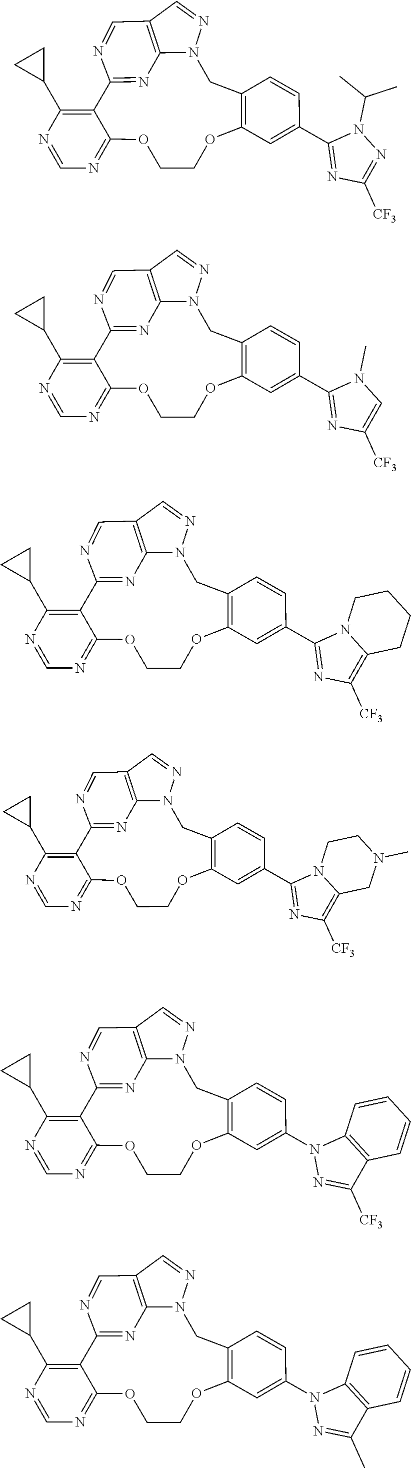 [SMILES: CC(C)n1nc(C(F)(F)F)nc1-c1ccc2c(c1)OCCOc1ncnc(C3CC3)c1-c1ncc3cnn(c3n1)C2 ; Cn1cc(C(F)(F)F)nc1-c1ccc2c(c1)OCCOc1ncnc(C3CC3)c1-c1ncc3cnn(c3n1)C2 ; FC(F)(F)c1nc(-c2ccc3c(c2)OCCOc2ncnc(C4CC4)c2-c2ncc4cnn(c4n2)C3)n2c1CCCC2 ; CN1CCn2c(-c3ccc4c(c3)OCCOc3ncnc(C5CC5)c3-c3ncc5cnn(c5n3)C4)nc(C(F)(F)F)c2C1 ; FC(F)(F)c1nn(-c2ccc3c(c2)OCCOc2ncnc(C4CC4)c2-c2ncc4cnn(c4n2)C3)c2ccccc12 ; Cc1nn(-c2ccc3c(c2)OCCOc2ncnc(C4CC4)c2-c2ncc4cnn(c4n2)C3)c2ccccc12]

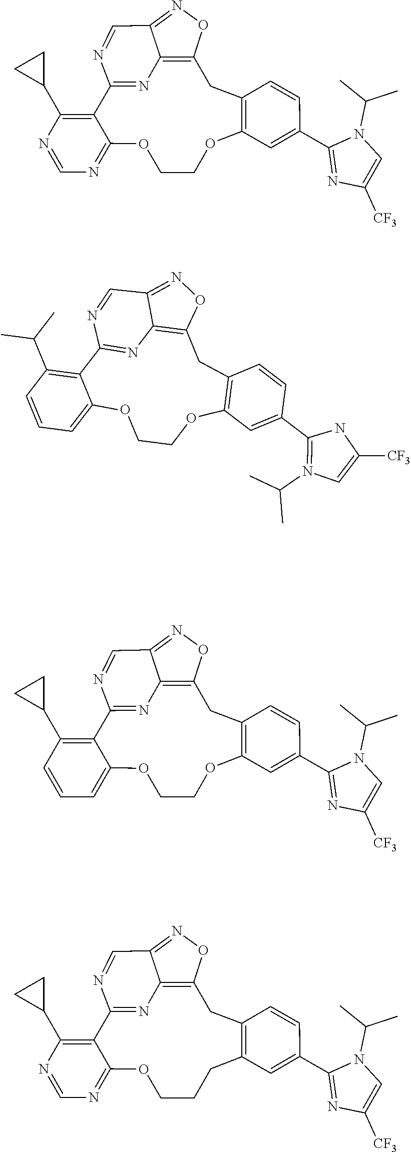

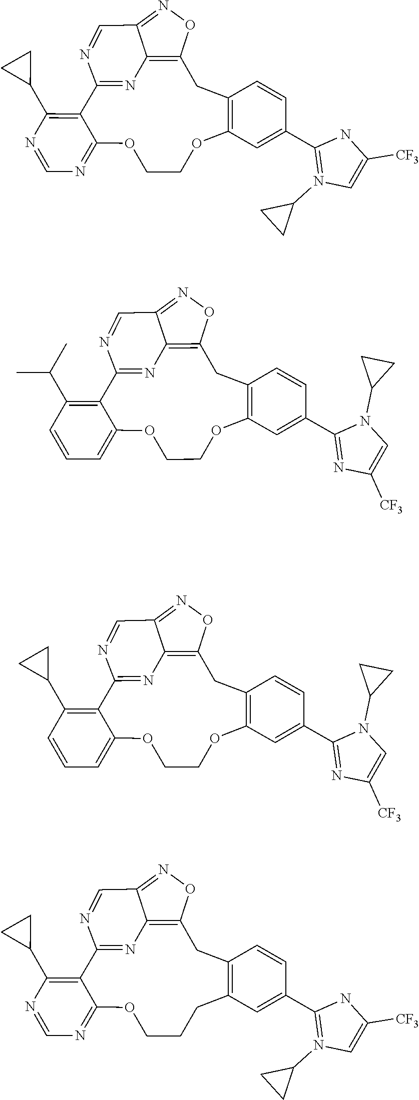

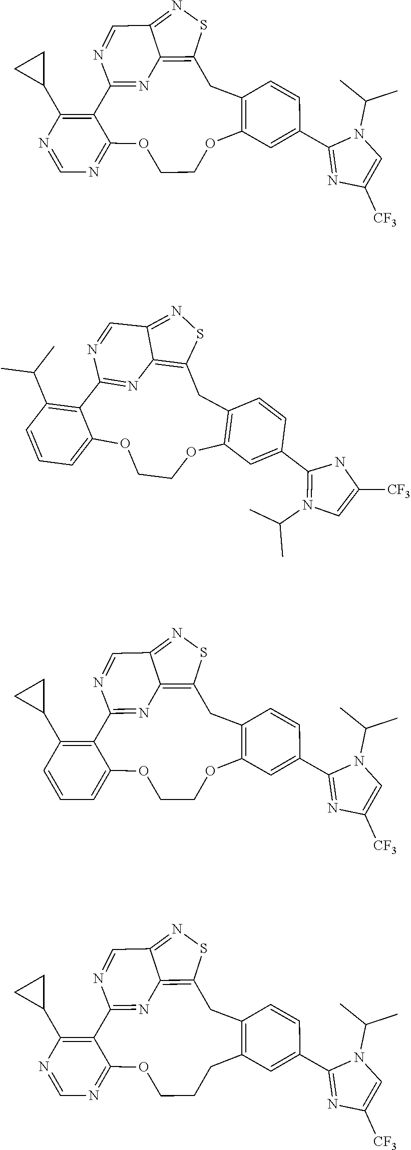

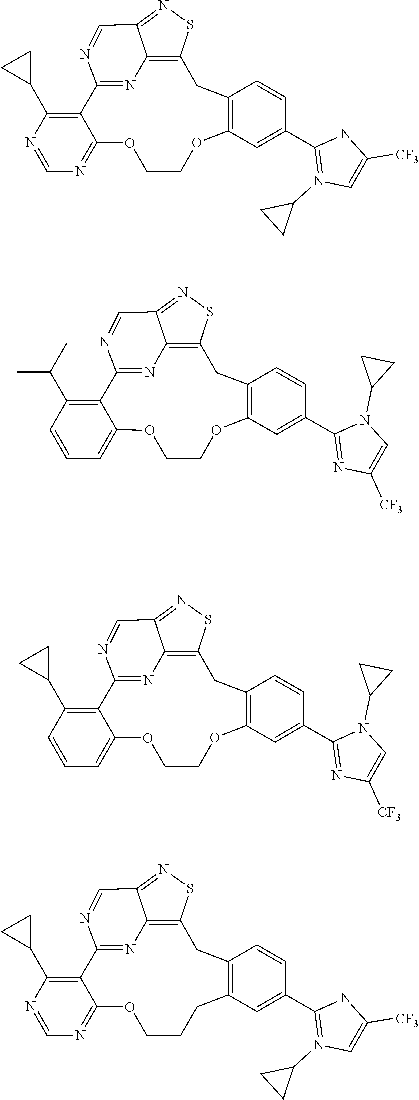

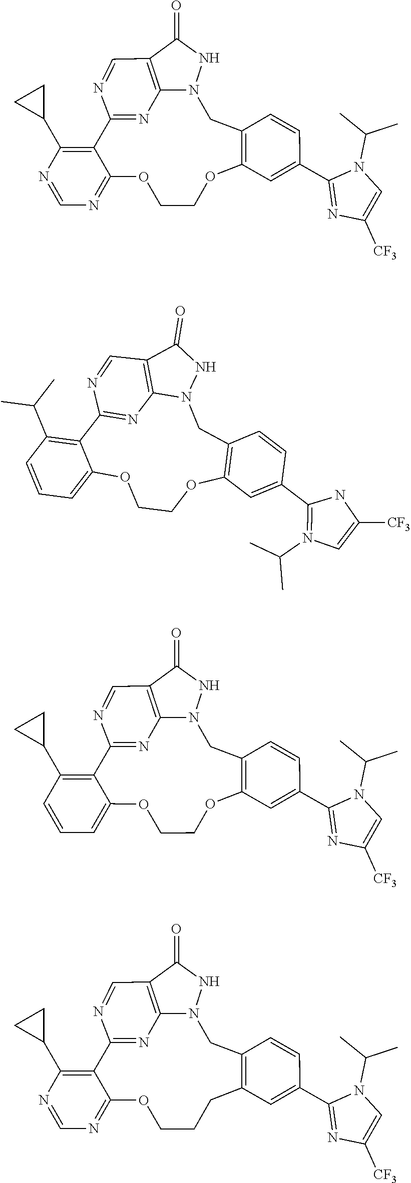

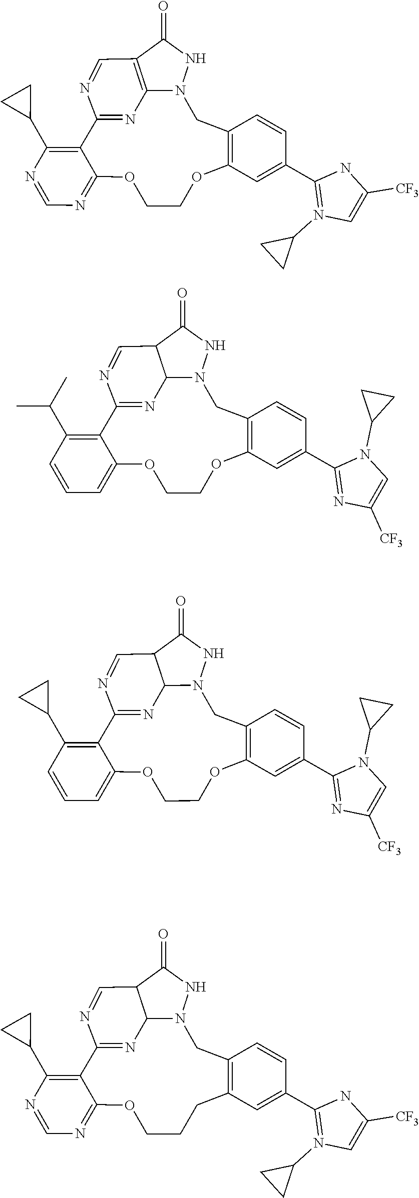

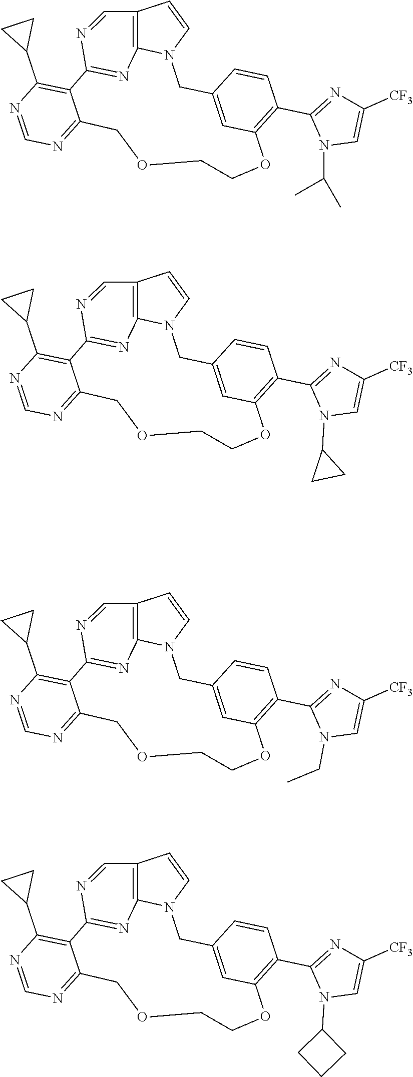

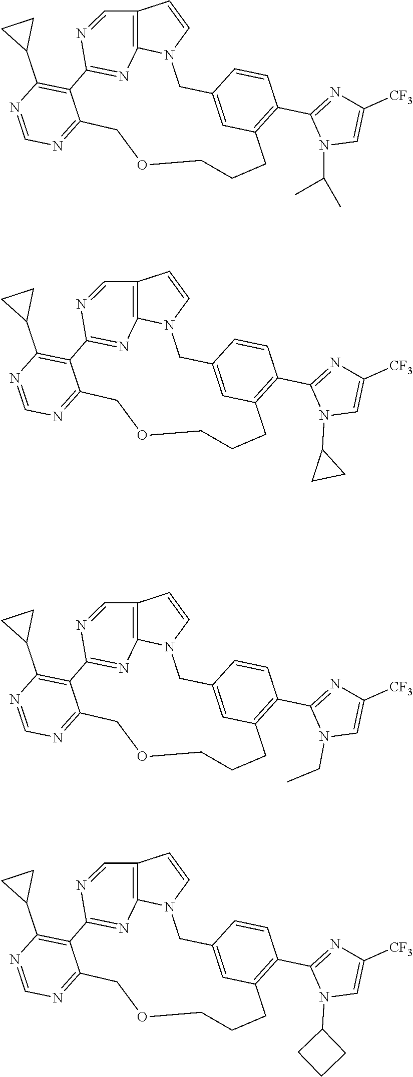

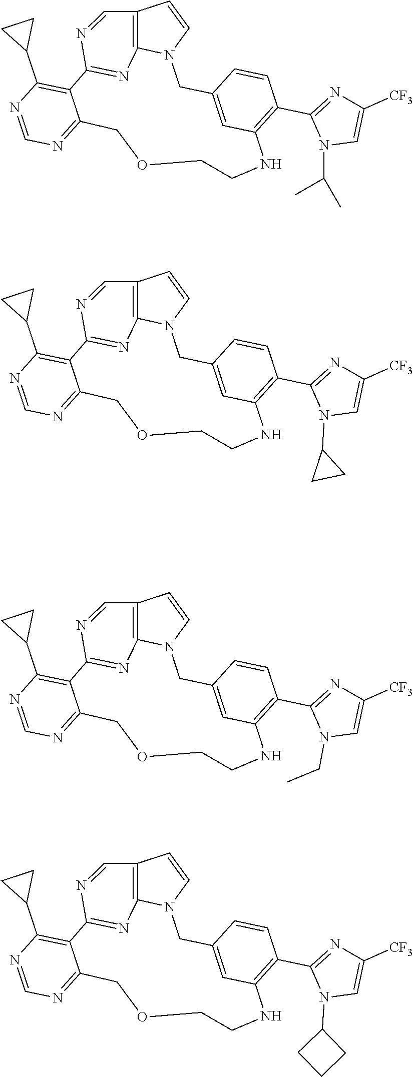

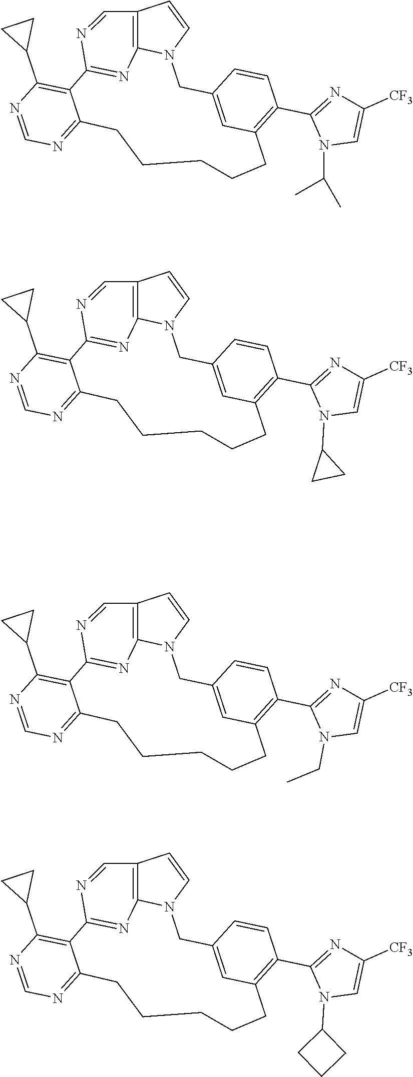

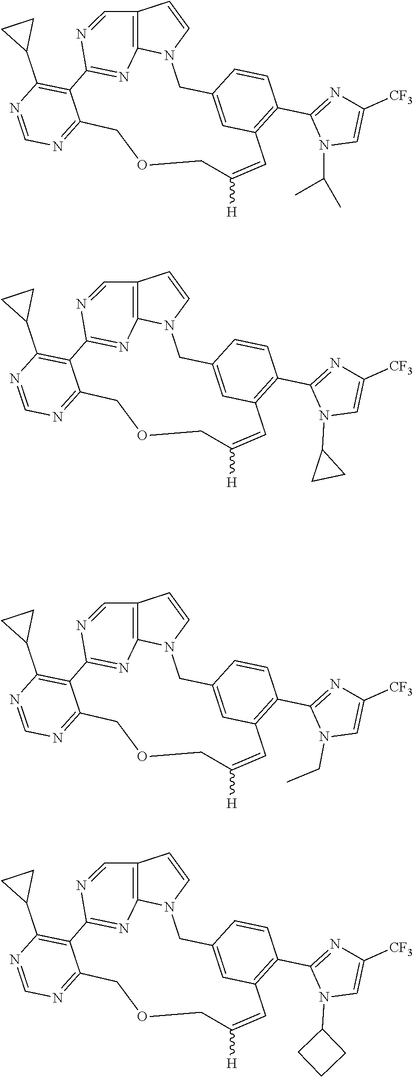

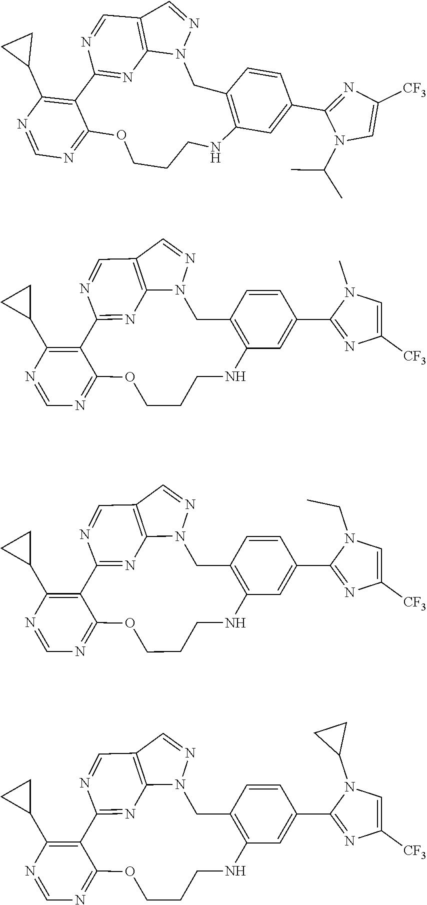 [SMILES: CC(C)n1cc(C(F)(F)F)nc1-c1ccc2c(c1)NCCCOc1ncnc(C3CC3)c1-c1ncc3cnn(c3n1)C2 ; Cn1cc(C(F)(F)F)nc1-c1ccc2c(c1)NCCCOc1ncnc(C3CC3)c1-c1ncc3cnn(c3n1)C2 ; CCn1cc(C(F)(F)F)nc1-c1ccc2c(c1)NCCCOc1ncnc(C3CC3)c1-c1ncc3cnn(c3n1)C2 ; FC(F)(F)c1cn(C2CC2)c(-c2ccc3c(c2)NCCCOc2ncnc(C4CC4)c2-c2ncc4cnn(c4n2)C3)n1]

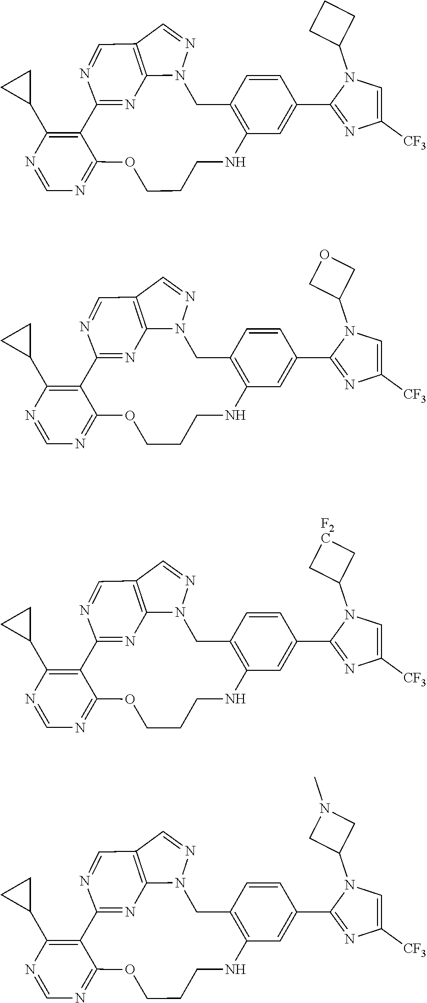 [SMILES: FC(F)(F)c1cn(C2CCC2)c(-c2ccc3c(c2)NCCCOc2ncnc(C4CC4)c2-c2ncc4cnn(c4n2)C3)n1 ; FC(F)(F)c1cn(C2COC2)c(-c2ccc3c(c2)NCCCOc2ncnc(C4CC4)c2-c2ncc4cnn(c4n2)C3)n1 ; FC1(F)CC(n2cc(C(F)(F)F)nc2-c2ccc3c(c2)NCCCOc2ncnc(C4CC4)c2-c2ncc4cnn(c4n2)C3)C1 ; CN1CC(n2cc(C(F)(F)F)nc2-c2ccc3c(c2)NCCCOc2ncnc(C4CC4)c2-c2ncc4cnn(c4n2)C3)C1]

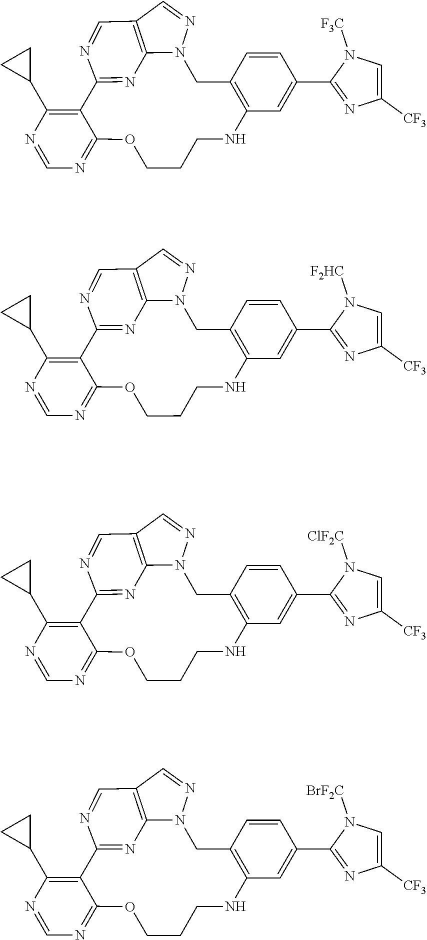

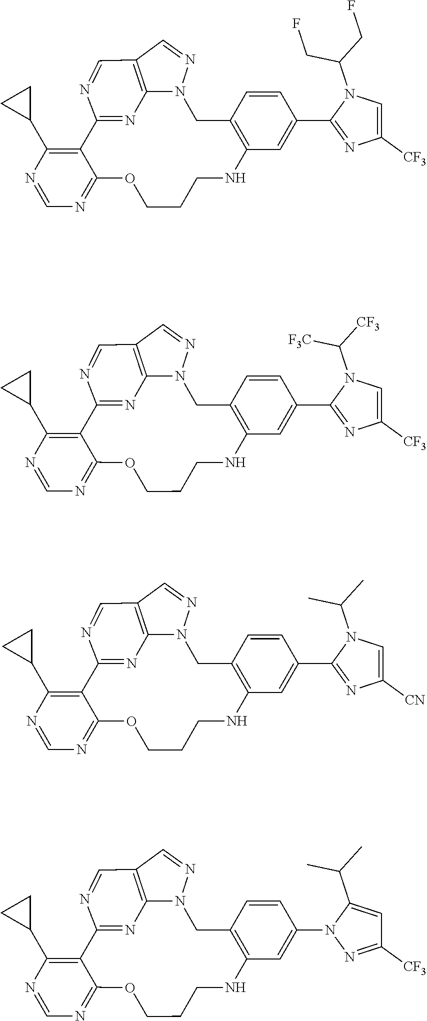 [SMILES: FCC(CF)n1cc(C(F)(F)F)nc1-c1ccc2c(c1)NCCCOc1ncnc(C3CC3)c1-c1ncc3cnn(c3n1)C2 ; FC(F)(F)c1cn(C(C(F)(F)F)C(F)(F)F)c(-c2ccc3c(c2)NCCCOc2ncnc(C4CC4)c2-c2ncc4cnn(c4n2)C3)n1 ; CC(C)n1cc(C#N)nc1-c1ccc2c(c1)NCCCOc1ncnc(C3CC3)c1-c1ncc3cnn(c3n1)C2 ; CC(C)c1cc(C(F)(F)F)nn1-c1ccc2c(c1)NCCCOc1ncnc(C3CC3)c1-c1ncc3cnn(c3n1)C2]

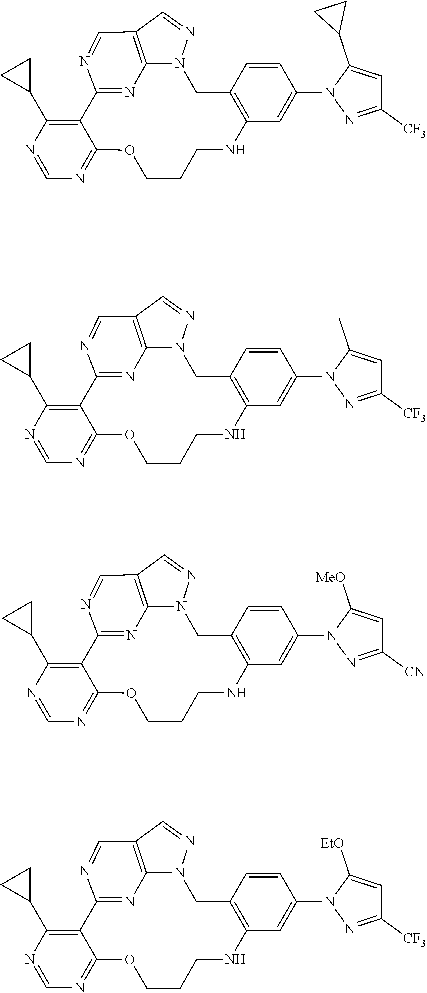 [SMILES: FC(F)(F)c1cc(C2CC2)n(-c2ccc3c(c2)NCCCOc2ncnc(C4CC4)c2-c2ncc4cnn(c4n2)C3)n1 ; Cc1cc(C(F)(F)F)nn1-c1ccc2c(c1)NCCCOc1ncnc(C3CC3)c1-c1ncc3cnn(c3n1)C2 ; COc1cc(C#N)nn1-c1ccc2c(c1)NCCCOc1ncnc(C3CC3)c1-c1ncc3cnn(c3n1)C2 ; CCOc1cc(C(F)(F)F)nn1-c1ccc2c(c1)NCCCOc1ncnc(C3CC3)c1-c1ncc3cnn(c3n1)C2]

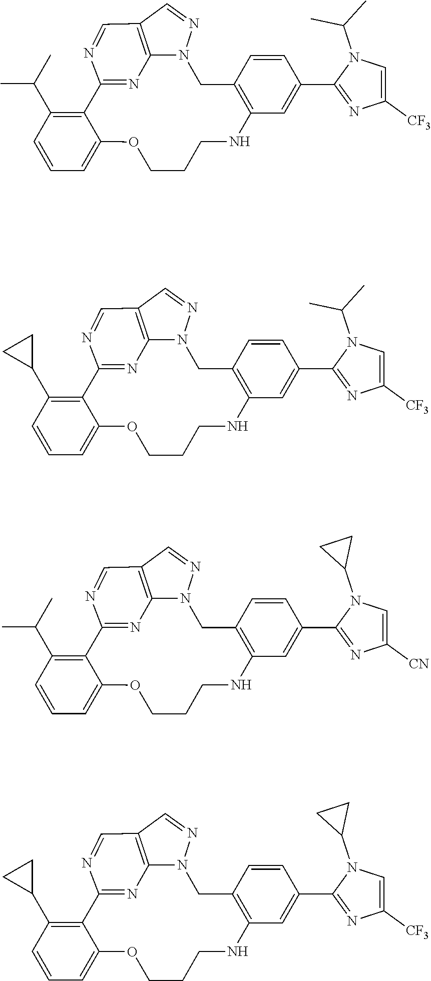 [SMILES: CC(C)c1cccc2c1-c1ncc3cnn(c3n1)Cc1ccc(-c3nc(C(F)(F)F)cn3C(C)C)cc1NCCCO2 ; CC(C)n1cc(C(F)(F)F)nc1-c1ccc2c(c1)NCCCOc1cccc(C3CC3)c1-c1ncc3cnn(c3n1)C2 ; CC(C)c1cccc2c1-c1ncc3cnn(c3n1)Cc1ccc(-c3nc(C#N)cn3C3CC3)cc1NCCCO2 ; FC(F)(F)c1cn(C2CC2)c(-c2ccc3c(c2)NCCCOc2cccc(C4CC4)c2-c2ncc4cnn(c4n2)C3)n1]

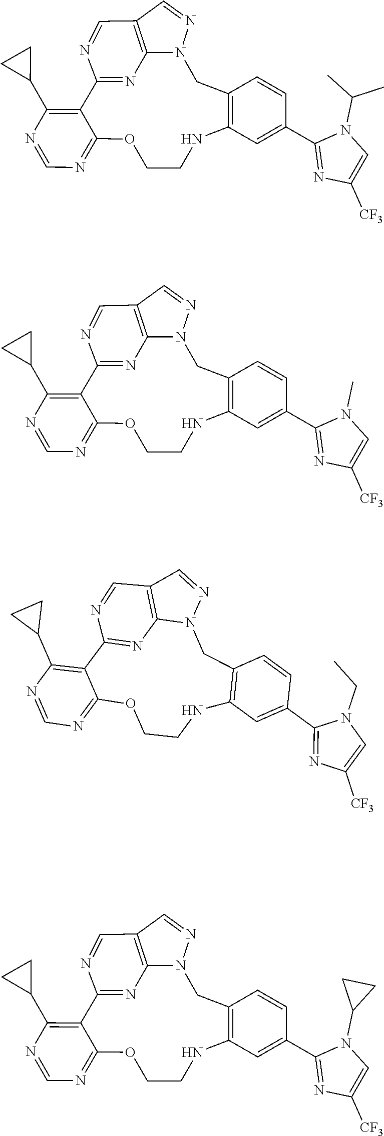 [SMILES: CC(C)n1cc(C(F)(F)F)nc1-c1ccc2c(c1)NCCOc1ncnc(C3CC3)c1-c1ncc3cnn(c3n1)C2 ; Cn1cc(C(F)(F)F)nc1-c1ccc2c(c1)NCCOc1ncnc(C3CC3)c1-c1ncc3cnn(c3n1)C2 ; CCn1cc(C(F)(F)F)nc1-c1ccc2c(c1)NCCOc1ncnc(C3CC3)c1-c1ncc3cnn(c3n1)C2 ; FC(F)(F)c1cn(C2CC2)c(-c2ccc3c(c2)NCCOc2ncnc(C4CC4)c2-c2ncc4cnn(c4n2)C3)n1]

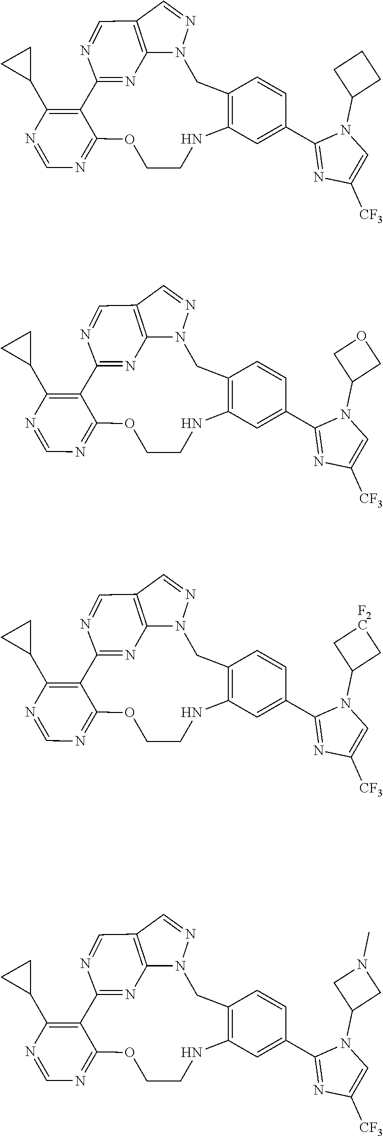 [SMILES: FC(F)(F)c1cn(C2CCC2)c(-c2ccc3c(c2)NCCOc2ncnc(C4CC4)c2-c2ncc4cnn(c4n2)C3)n1 ; FC(F)(F)c1cn(C2COC2)c(-c2ccc3c(c2)NCCOc2ncnc(C4CC4)c2-c2ncc4cnn(c4n2)C3)n1 ; FC1(F)CC(n2cc(C(F)(F)F)nc2-c2ccc3c(c2)NCCOc2ncnc(C4CC4)c2-c2ncc4cnn(c4n2)C3)C1 ; CN1CC(n2cc(C(F)(F)F)nc2-c2ccc3c(c2)NCCOc2ncnc(C4CC4)c2-c2ncc4cnn(c4n2)C3)C1]

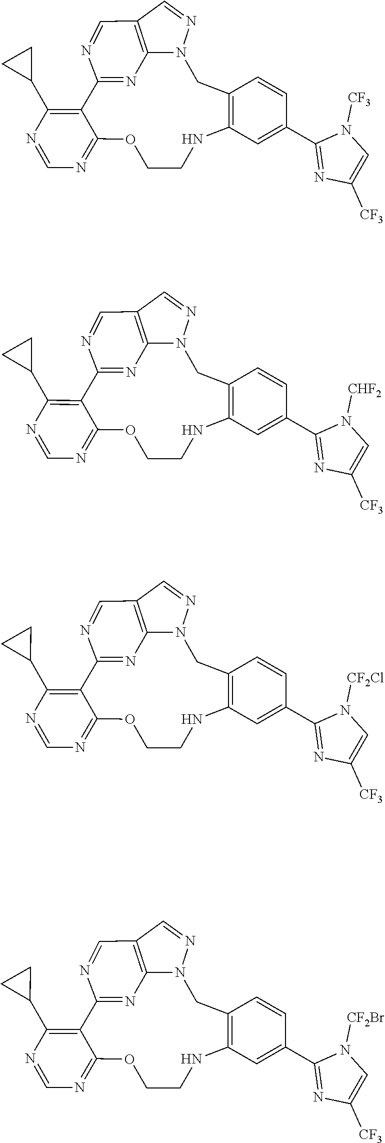

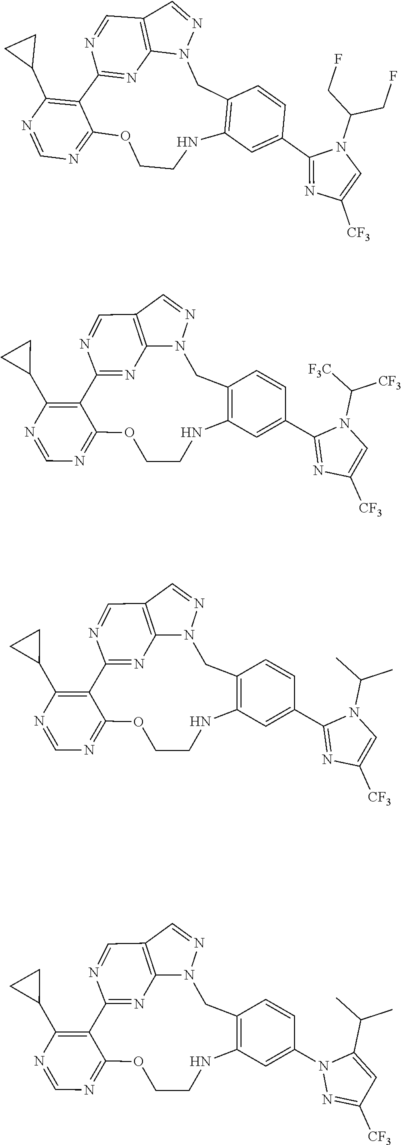 [SMILES: FCC(CF)n1cc(C(F)(F)F)nc1-c1ccc2c(c1)NCCOc1ncnc(C3CC3)c1-c1ncc3cnn(c3n1)C2 ; FC(F)(F)c1cn(C(C(F)(F)F)C(F)(F)F)c(-c2ccc3c(c2)NCCOc2ncnc(C4CC4)c2-c2ncc4cnn(c4n2)C3)n1 ; CC(C)n1cc(C(F)(F)F)nc1-c1ccc2c(c1)NCCOc1ncnc(C3CC3)c1-c1ncc3cnn(c3n1)C2 ; CC(C)c1cc(C(F)(F)F)nn1-c1ccc2c(c1)NCCOc1ncnc(C3CC3)c1-c1ncc3cnn(c3n1)C2]

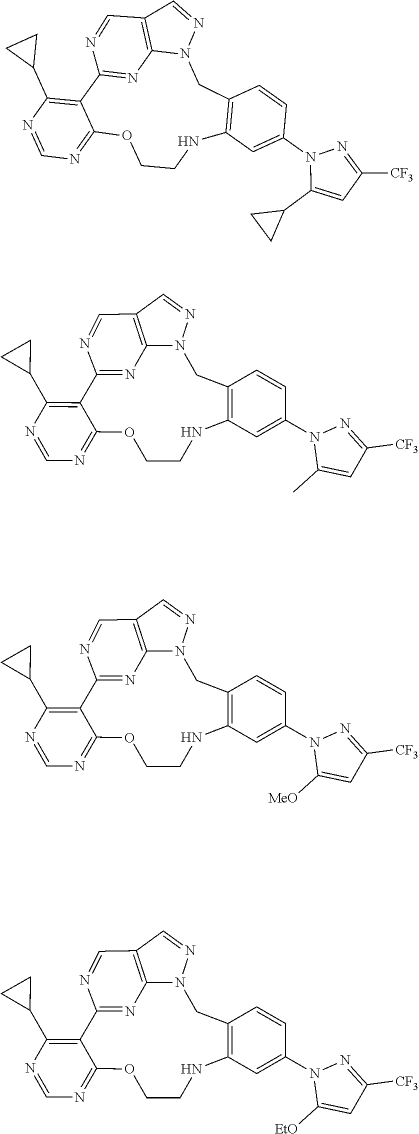 [SMILES: FC(F)(F)c1cc(C2CC2)n(-c2ccc3c(c2)NCCOc2ncnc(C4CC4)c2-c2ncc4cnn(c4n2)C3)n1 ; Cc1cc(C(F)(F)F)nn1-c1ccc2c(c1)NCCOc1ncnc(C3CC3)c1-c1ncc3cnn(c3n1)C2 ; COc1cc(C(F)(F)F)nn1-c1ccc2c(c1)NCCOc1ncnc(C3CC3)c1-c1ncc3cnn(c3n1)C2 ; CCOc1cc(C(F)(F)F)nn1-c1ccc2c(c1)NCCOc1ncnc(C3CC3)c1-c1ncc3cnn(c3n1)C2]

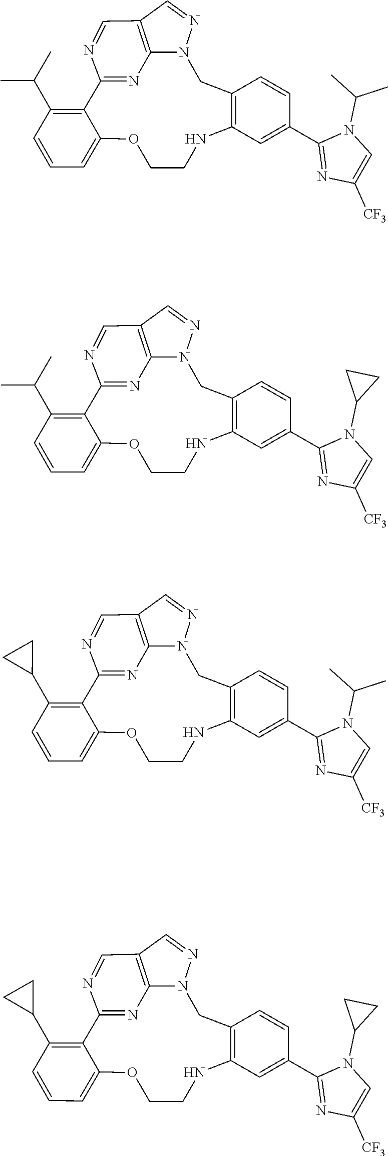

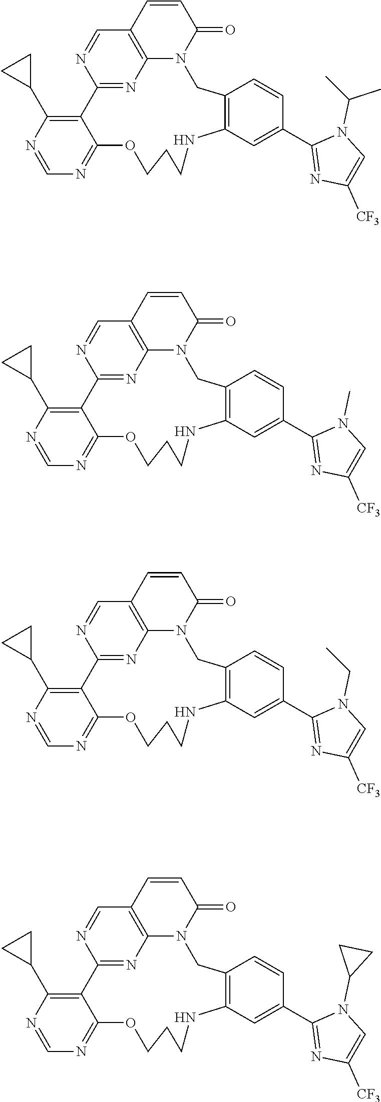 [SMILES: CC(C)n1cc(C(F)(F)F)nc1-c1ccc2c(c1)NCCCOc1ncnc(C3CC3)c1-c1ncc3ccc(=O)n(c3n1)C2 ; Cn1cc(C(F)(F)F)nc1-c1ccc2c(c1)NCCCOc1ncnc(C3CC3)c1-c1ncc3ccc(=O)n(c3n1)C2 ; CCn1cc(C(F)(F)F)nc1-c1ccc2c(c1)NCCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1NCCCOc1ncnc(C2CC2)c1-3]

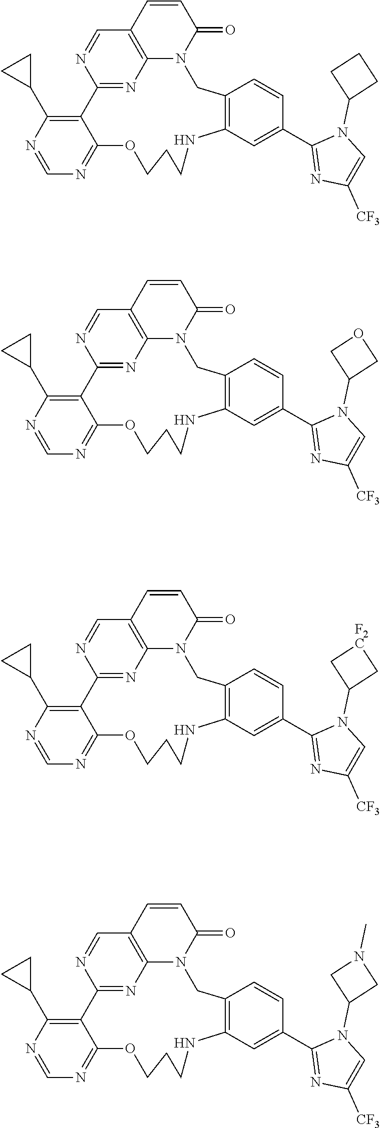 [SMILES: O=c1ccc2cnc3nc2n1Cc1ccc(-c2nc(C(F)(F)F)cn2C2CCC2)cc1NCCCOc1ncnc(C2CC2)c1-3 ; O=c1ccc2cnc3nc2n1Cc1ccc(-c2nc(C(F)(F)F)cn2C2COC2)cc1NCCCOc1ncnc(C2CC2)c1-3 ; O=c1ccc2cnc3nc2n1Cc1ccc(-c2nc(C(F)(F)F)cn2C2CC(F)(F)C2)cc1NCCCOc1ncnc(C2CC2)c1-3 ; CN1CC(n2cc(C(F)(F)F)nc2-c2ccc3c(c2)NCCCOc2ncnc(C4CC4)c2-c2ncc4ccc(=O)n(c4n2)C3)C1]

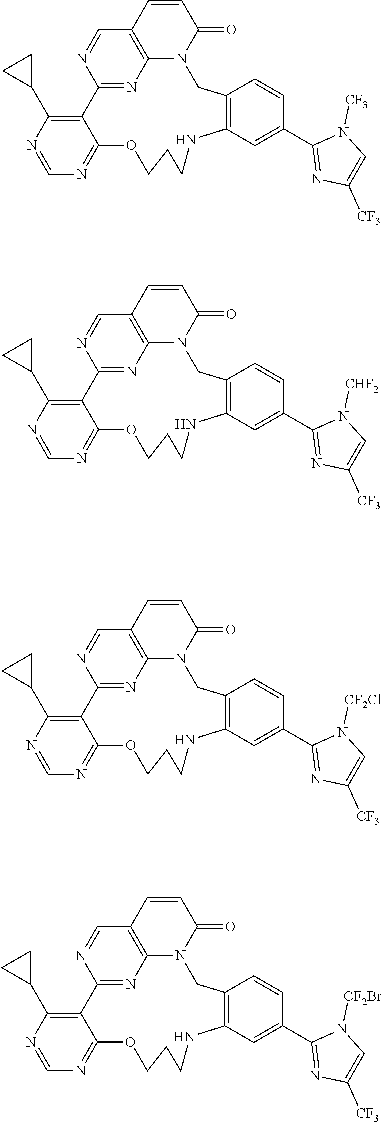

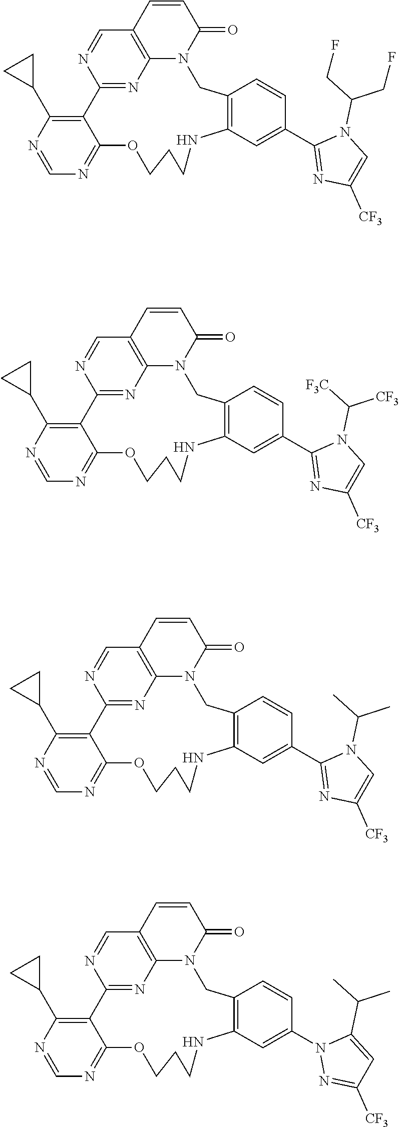 [SMILES: O=c1ccc2cnc3nc2n1Cc1ccc(-c2nc(C(F)(F)F)cn2C(CF)CF)cc1NCCCOc1ncnc(C2CC2)c1-3 ; O=c1ccc2cnc3nc2n1Cc1ccc(-c2nc(C(F)(F)F)cn2C(C(F)(F)F)C(F)(F)F)cc1NCCCOc1ncnc(C2CC2)c1-3 ; CC(C)n1cc(C(F)(F)F)nc1-c1ccc2c(c1)NCCCOc1ncnc(C3CC3)c1-c1ncc3ccc(=O)n(c3n1)C2 ; CC(C)c1cc(C(F)(F)F)nn1-c1ccc2c(c1)NCCCOc1ncnc(C3CC3)c1-c1ncc3ccc(=O)n(c3n1)C2]

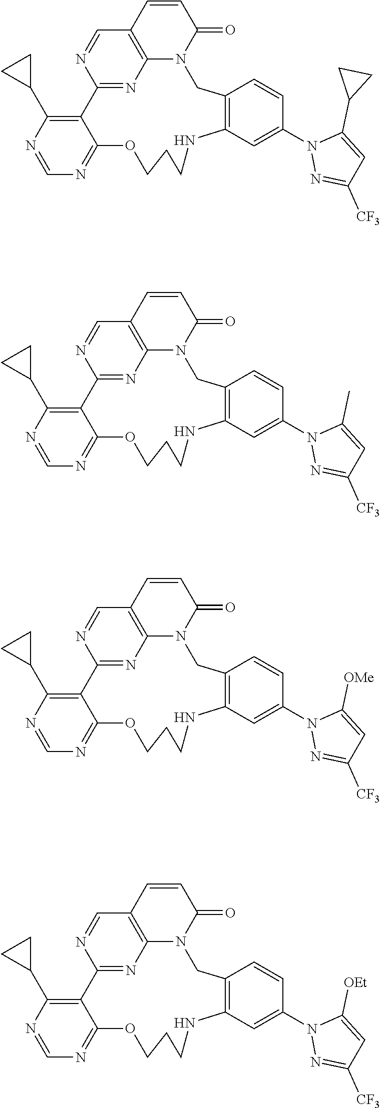 [SMILES: O=c1ccc2cnc3nc2n1Cc1ccc(-n2nc(C(F)(F)F)cc2C2CC2)cc1NCCCOc1ncnc(C2CC2)c1-3 ; Cc1cc(C(F)(F)F)nn1-c1ccc2c(c1)NCCCOc1ncnc(C3CC3)c1-c1ncc3ccc(=O)n(c3n1)C2 ; COc1cc(C(F)(F)F)nn1-c1ccc2c(c1)NCCCOc1ncnc(C3CC3)c1-c1ncc3ccc(=O)n(c3n1)C2 ; CCOc1cc(C(F)(F)F)nn1-c1ccc2c(c1)NCCCOc1ncnc(C3CC3)c1-c1ncc3ccc(=O)n(c3n1)C2]

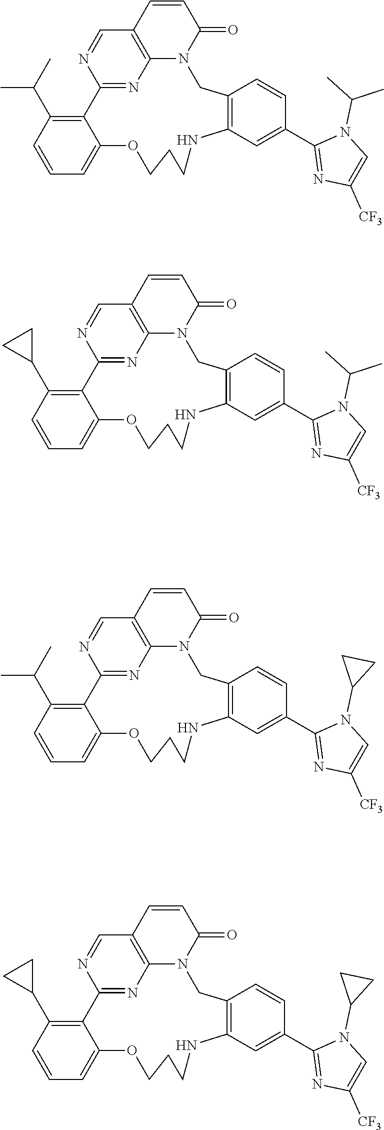

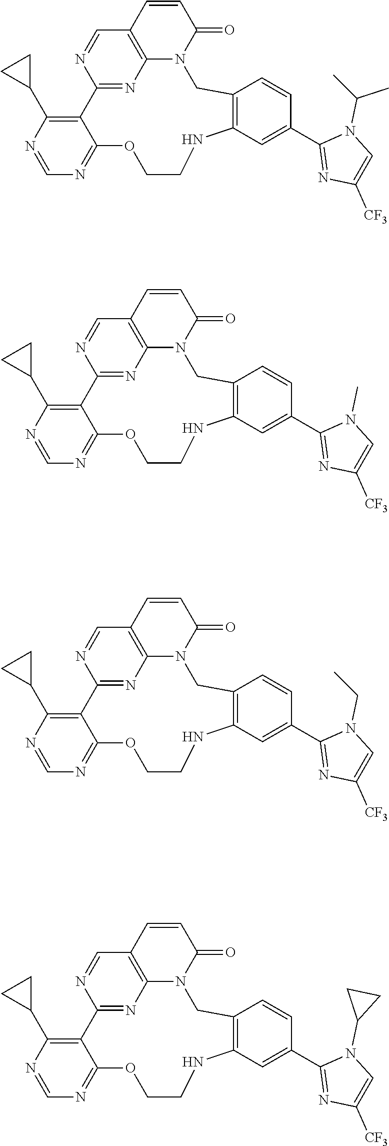 [SMILES: CC(C)n1cc(C(F)(F)F)nc1-c1ccc2c(c1)NCCOc1ncnc(C3CC3)c1-c1ncc3ccc(=O)n(c3n1)C2 ; Cn1cc(C(F)(F)F)nc1-c1ccc2c(c1)NCCOc1ncnc(C3CC3)c1-c1ncc3ccc(=O)n(c3n1)C2 ; CCn1cc(C(F)(F)F)nc1-c1ccc2c(c1)NCCOc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)cc1NCCOc1ncnc(C2CC2)c1-3]

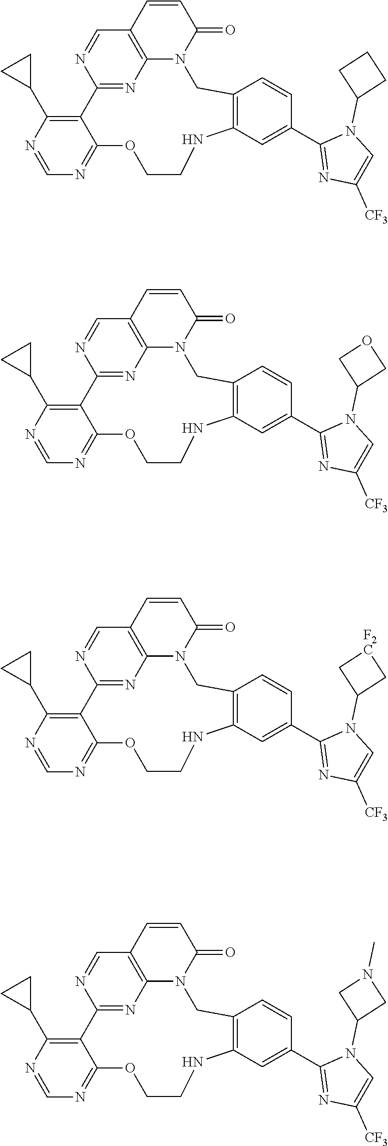 [SMILES: O=c1ccc2cnc3nc2n1Cc1ccc(-c2nc(C(F)(F)F)cn2C2CCC2)cc1NCCOc1ncnc(C2CC2)c1-3 ; O=c1ccc2cnc3nc2n1Cc1ccc(-c2nc(C(F)(F)F)cn2C2COC2)cc1NCCOc1ncnc(C2CC2)c1-3 ; O=c1ccc2cnc3nc2n1Cc1ccc(-c2nc(C(F)(F)F)cn2C2CC(F)(F)C2)cc1NCCOc1ncnc(C2CC2)c1-3 ; CN1CC(n2cc(C(F)(F)F)nc2-c2ccc3c(c2)NCCOc2ncnc(C4CC4)c2-c2ncc4ccc(=O)n(c4n2)C3)C1]

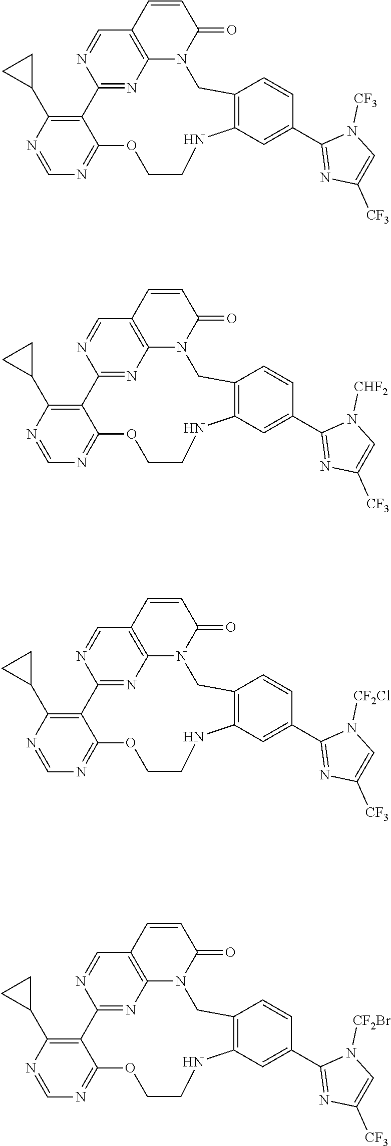

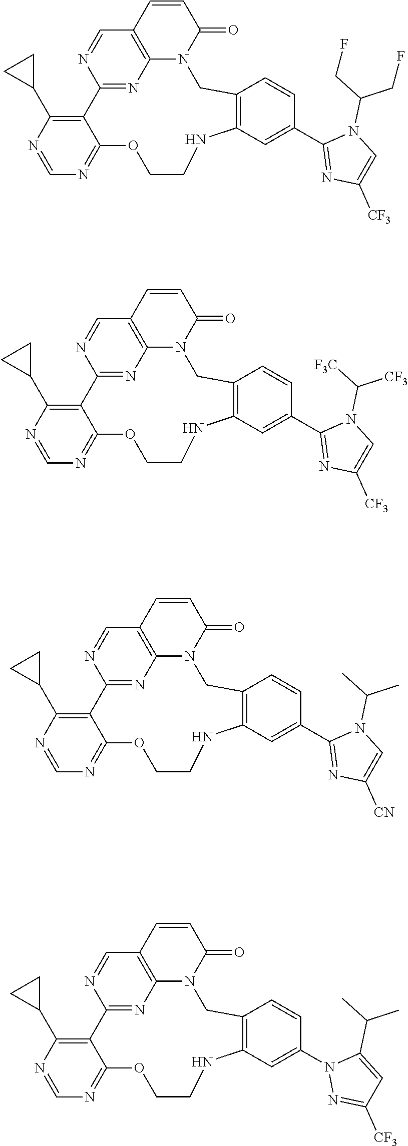 [SMILES: O=c1ccc2cnc3nc2n1Cc1ccc(-c2nc(C(F)(F)F)cn2C(CF)CF)cc1NCCOc1ncnc(C2CC2)c1-3 ; O=c1ccc2cnc3nc2n1Cc1ccc(-c2nc(C(F)(F)F)cn2C(C(F)(F)F)C(F)(F)F)cc1NCCOc1ncnc(C2CC2)c1-3 ; CC(C)n1cc(C#N)nc1-c1ccc2c(c1)NCCOc1ncnc(C3CC3)c1-c1ncc3ccc(=O)n(c3n1)C2 ; CC(C)c1cc(C(F)(F)F)nn1-c1ccc2c(c1)NCCOc1ncnc(C3CC3)c1-c1ncc3ccc(=O)n(c3n1)C2]

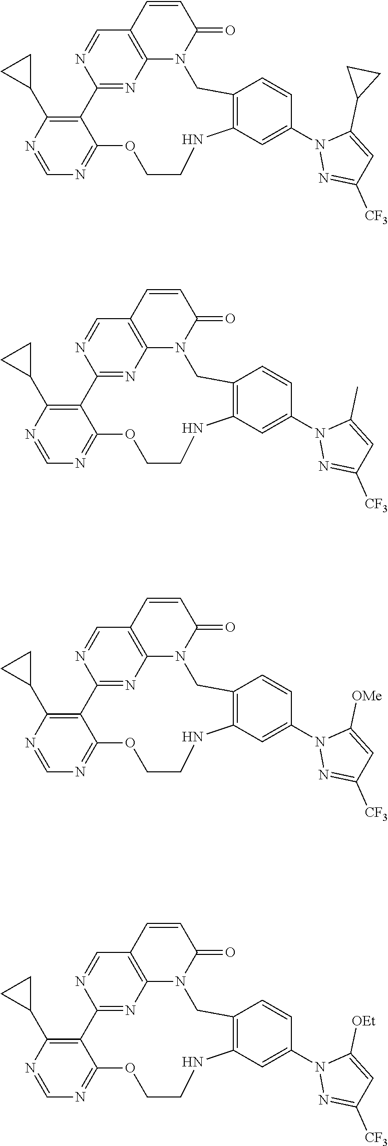 [SMILES: O=c1ccc2cnc3nc2n1Cc1ccc(-n2nc(C(F)(F)F)cc2C2CC2)cc1NCCOc1ncnc(C2CC2)c1-3 ; Cc1cc(C(F)(F)F)nn1-c1ccc2c(c1)NCCOc1ncnc(C3CC3)c1-c1ncc3ccc(=O)n(c3n1)C2 ; COc1cc(C(F)(F)F)nn1-c1ccc2c(c1)NCCOc1ncnc(C3CC3)c1-c1ncc3ccc(=O)n(c3n1)C2 ; CCOc1cc(C(F)(F)F)nn1-c1ccc2c(c1)NCCOc1ncnc(C3CC3)c1-c1ncc3ccc(=O)n(c3n1)C2]

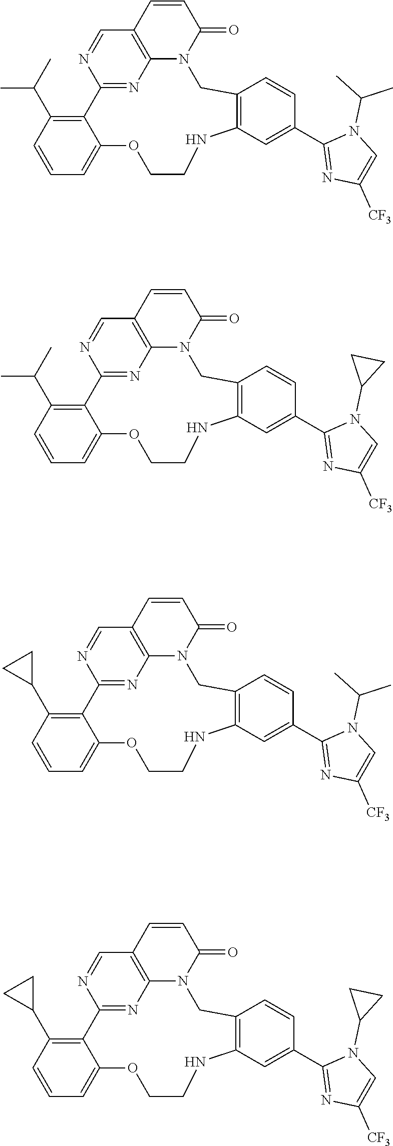

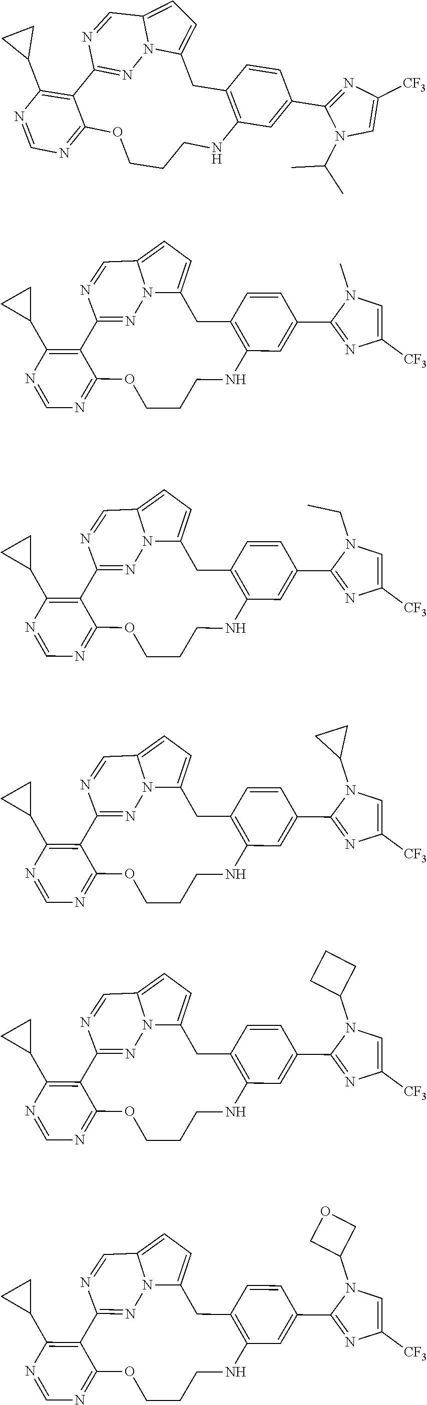 [SMILES: CC(C)n1cc(C(F)(F)F)nc1-c1ccc2c(c1)NCCCOc1ncnc(C3CC3)c1-c1ncc3ccc(n3n1)C2 ; Cn1cc(C(F)(F)F)nc1-c1ccc2c(c1)NCCCOc1ncnc(C3CC3)c1-c1ncc3ccc(n3n1)C2 ; CCn1cc(C(F)(F)F)nc1-c1ccc2c(c1)NCCCOc1ncnc(C3CC3)c1-c1ncc3ccc(n3n1)C2 ; FC(F)(F)c1cn(C2CC2)c(-c2ccc3c(c2)NCCCOc2ncnc(C4CC4)c2-c2ncc4ccc(n4n2)C3)n1 ; FC(F)(F)c1cn(C2CCC2)c(-c2ccc3c(c2)NCCCOc2ncnc(C4CC4)c2-c2ncc4ccc(n4n2)C3)n1 ; FC(F)(F)c1cn(C2COC2)c(-c2ccc3c(c2)NCCCOc2ncnc(C4CC4)c2-c2ncc4ccc(n4n2)C3)n1]

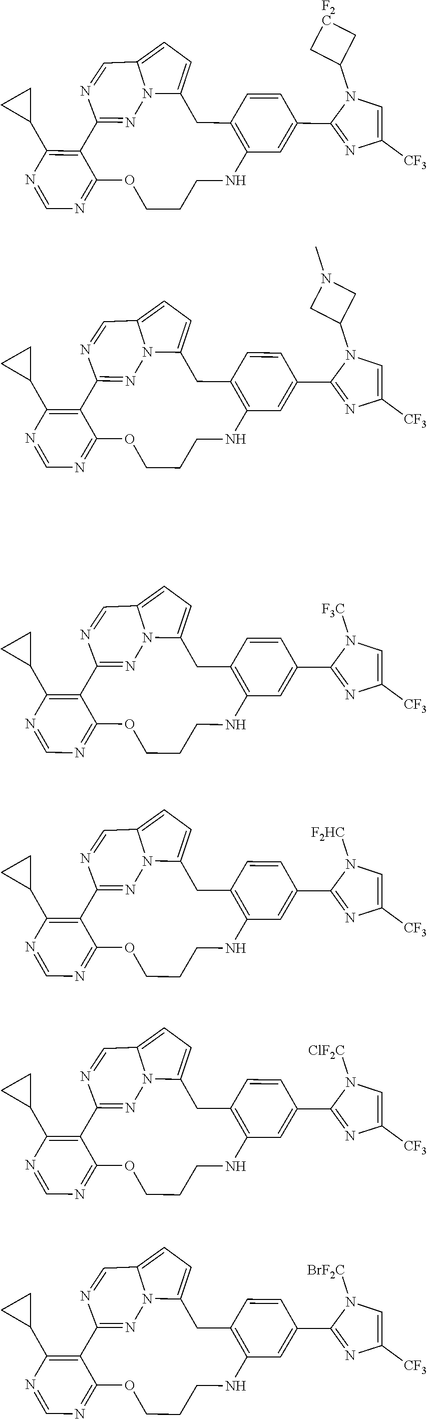

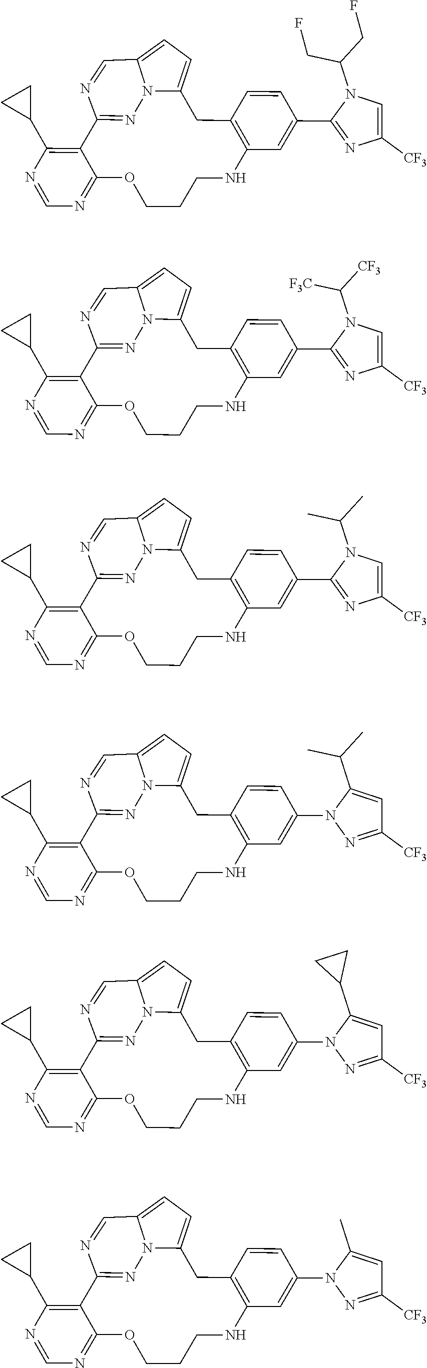 [SMILES: FCC(CF)n1cc(C(F)(F)F)nc1-c1ccc2c(c1)NCCCOc1ncnc(C3CC3)c1-c1ncc3ccc(n3n1)C2 ; FC(F)(F)c1cn(C(C(F)(F)F)C(F)(F)F)c(-c2ccc3c(c2)NCCCOc2ncnc(C4CC4)c2-c2ncc4ccc(n4n2)C3)n1 ; CC(C)n1cc(C(F)(F)F)nc1-c1ccc2c(c1)NCCCOc1ncnc(C3CC3)c1-c1ncc3ccc(n3n1)C2 ; CC(C)c1cc(C(F)(F)F)nn1-c1ccc2c(c1)NCCCOc1ncnc(C3CC3)c1-c1ncc3ccc(n3n1)C2 ; FC(F)(F)c1cc(C2CC2)n(-c2ccc3c(c2)NCCCOc2ncnc(C4CC4)c2-c2ncc4ccc(n4n2)C3)n1 ; Cc1cc(C(F)(F)F)nn1-c1ccc2c(c1)NCCCOc1ncnc(C3CC3)c1-c1ncc3ccc(n3n1)C2]

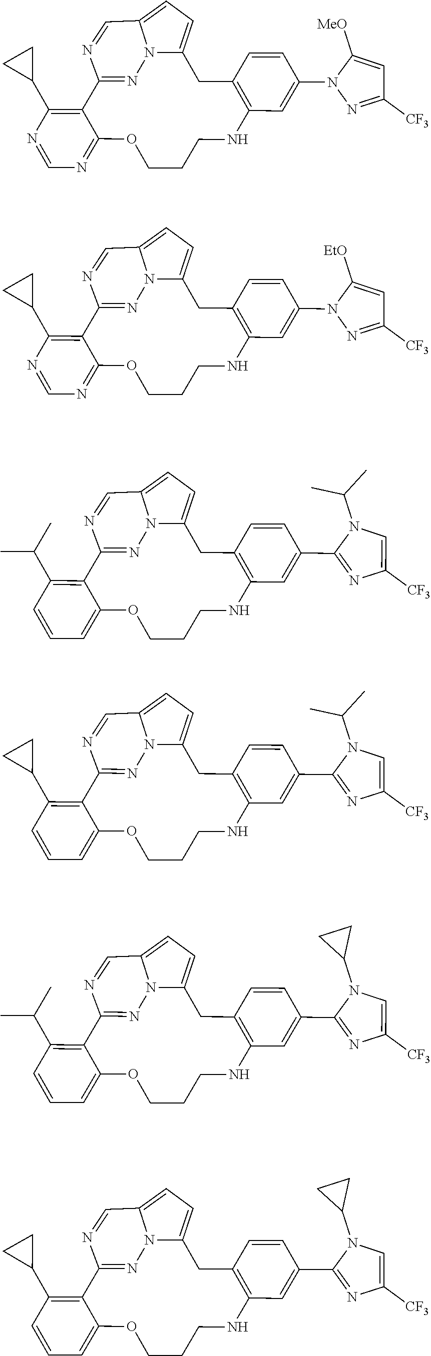 [SMILES: COc1cc(C(F)(F)F)nn1-c1ccc2c(c1)NCCCOc1ncnc(C3CC3)c1-c1ncc3ccc(n3n1)C2 ; CCOc1cc(C(F)(F)F)nn1-c1ccc2c(c1)NCCCOc1ncnc(C3CC3)c1-c1ncc3ccc(n3n1)C2 ; CC(C)c1cccc2c1-c1ncc3ccc(n3n1)Cc1ccc(-c3nc(C(F)(F)F)cn3C(C)C)cc1NCCCO2 ; CC(C)n1cc(C(F)(F)F)nc1-c1ccc2c(c1)NCCCOc1cccc(C3CC3)c1-c1ncc3ccc(n3n1)C2 ; CC(C)c1cccc2c1-c1ncc3ccc(n3n1)Cc1ccc(-c3nc(C(F)(F)F)cn3C3CC3)cc1NCCCO2 ; FC(F)(F)c1cn(C2CC2)c(-c2ccc3c(c2)NCCCOc2cccc(C4CC4)c2-c2ncc4ccc(n4n2)C3)n1]

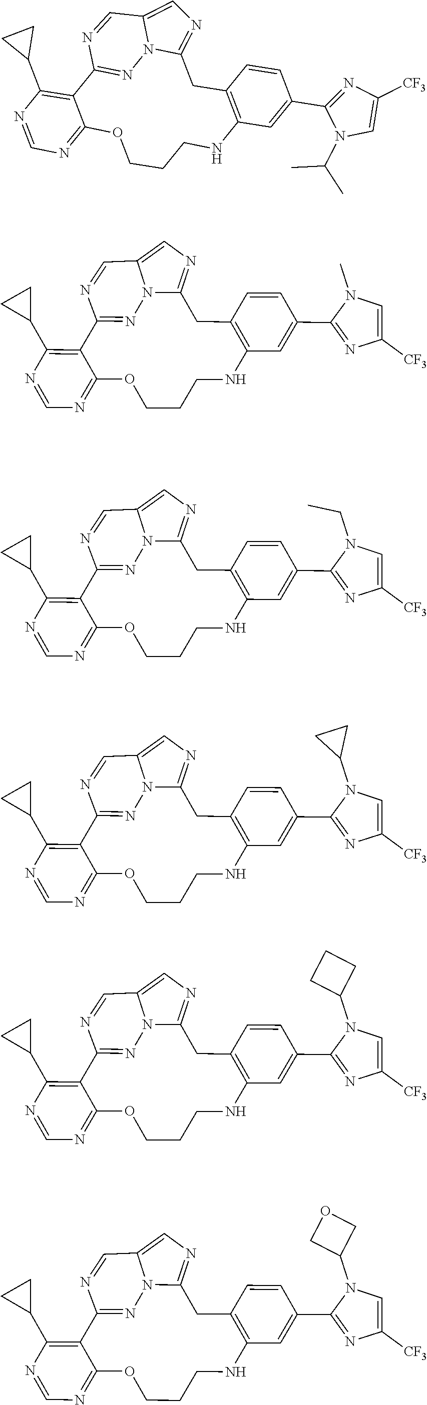 [SMILES: CC(C)n1cc(C(F)(F)F)nc1-c1ccc2c(c1)NCCCOc1ncnc(C3CC3)c1-c1ncc3cnc(n3n1)C2 ; Cn1cc(C(F)(F)F)nc1-c1ccc2c(c1)NCCCOc1ncnc(C3CC3)c1-c1ncc3cnc(n3n1)C2 ; CCn1cc(C(F)(F)F)nc1-c1ccc2c(c1)NCCCOc1ncnc(C3CC3)c1-c1ncc3cnc(n3n1)C2 ; FC(F)(F)c1cn(C2CC2)c(-c2ccc3c(c2)NCCCOc2ncnc(C4CC4)c2-c2ncc4cnc(n4n2)C3)n1 ; FC(F)(F)c1cn(C2CCC2)c(-c2ccc3c(c2)NCCCOc2ncnc(C4CC4)c2-c2ncc4cnc(n4n2)C3)n1 ; FC(F)(F)c1cn(C2COC2)c(-c2ccc3c(c2)NCCCOc2ncnc(C4CC4)c2-c2ncc4cnc(n4n2)C3)n1]

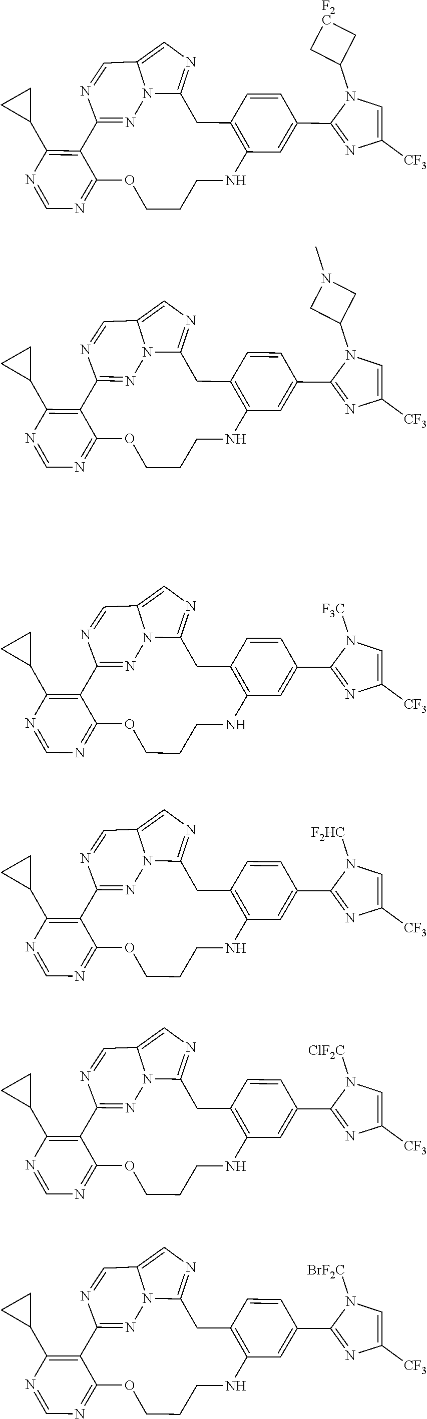

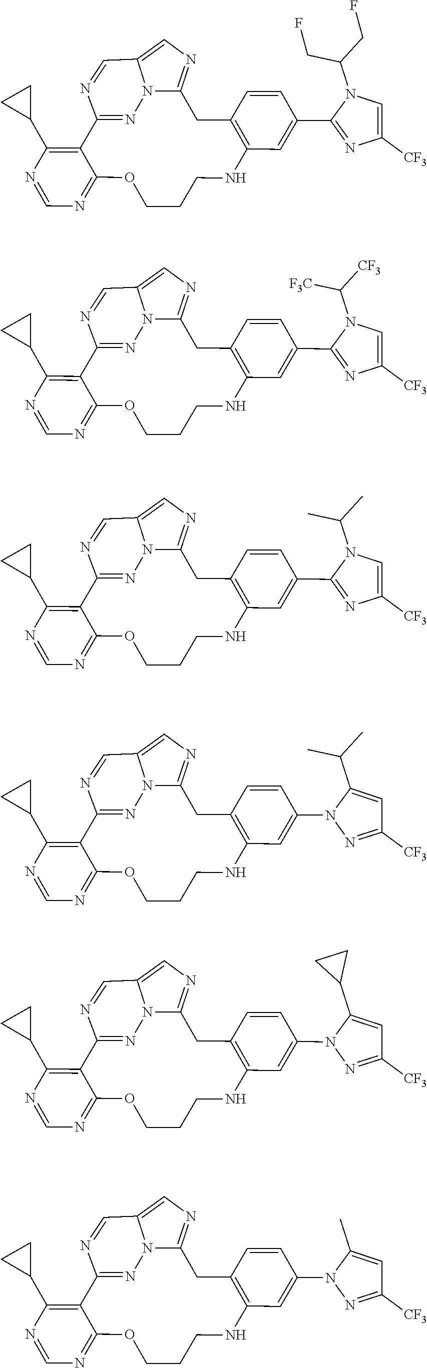 [SMILES: FCC(CF)n1cc(C(F)(F)F)nc1-c1ccc2c(c1)NCCCOc1ncnc(C3CC3)c1-c1ncc3cnc(n3n1)C2 ; FC(F)(F)c1cn(C(C(F)(F)F)C(F)(F)F)c(-c2ccc3c(c2)NCCCOc2ncnc(C4CC4)c2-c2ncc4cnc(n4n2)C3)n1 ; CC(C)n1cc(C(F)(F)F)nc1-c1ccc2c(c1)NCCCOc1ncnc(C3CC3)c1-c1ncc3cnc(n3n1)C2 ; CC(C)c1cc(C(F)(F)F)nn1-c1ccc2c(c1)NCCCOc1ncnc(C3CC3)c1-c1ncc3cnc(n3n1)C2 ; FC(F)(F)c1cc(C2CC2)n(-c2ccc3c(c2)NCCCOc2ncnc(C4CC4)c2-c2ncc4cnc(n4n2)C3)n1 ; Cc1cc(C(F)(F)F)nn1-c1ccc2c(c1)NCCCOc1ncnc(C3CC3)c1-c1ncc3cnc(n3n1)C2]

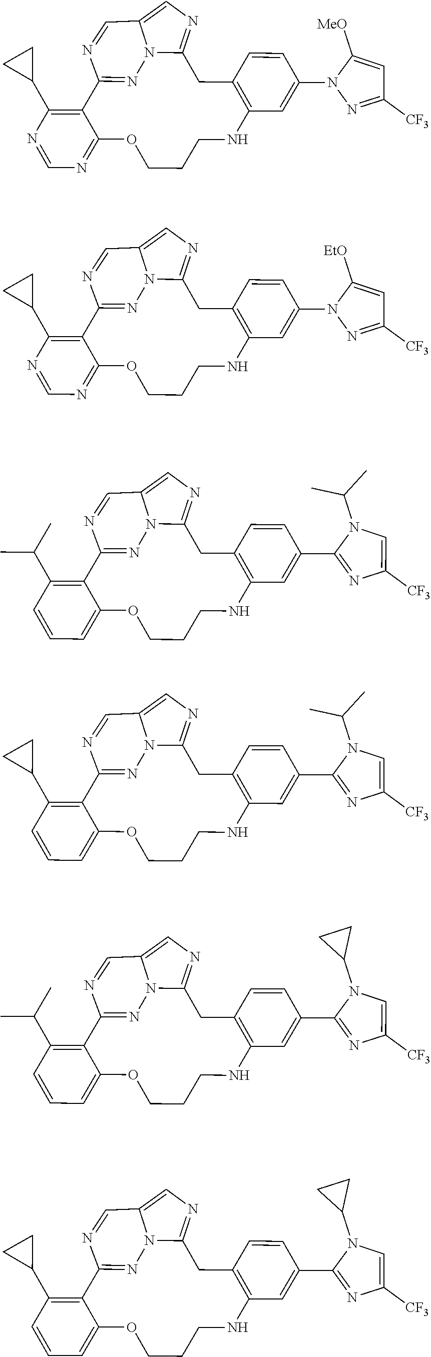 [SMILES: COc1cc(C(F)(F)F)nn1-c1ccc2c(c1)NCCCOc1ncnc(C3CC3)c1-c1ncc3cnc(n3n1)C2 ; CCOc1cc(C(F)(F)F)nn1-c1ccc2c(c1)NCCCOc1ncnc(C3CC3)c1-c1ncc3cnc(n3n1)C2 ; CC(C)c1cccc2c1-c1ncc3cnc(n3n1)Cc1ccc(-c3nc(C(F)(F)F)cn3C(C)C)cc1NCCCO2 ; CC(C)n1cc(C(F)(F)F)nc1-c1ccc2c(c1)NCCCOc1cccc(C3CC3)c1-c1ncc3cnc(n3n1)C2 ; CC(C)c1cccc2c1-c1ncc3cnc(n3n1)Cc1ccc(-c3nc(C(F)(F)F)cn3C3CC3)cc1NCCCO2 ; FC(F)(F)c1cn(C2CC2)c(-c2ccc3c(c2)NCCCOc2cccc(C4CC4)c2-c2ncc4cnc(n4n2)C3)n1]

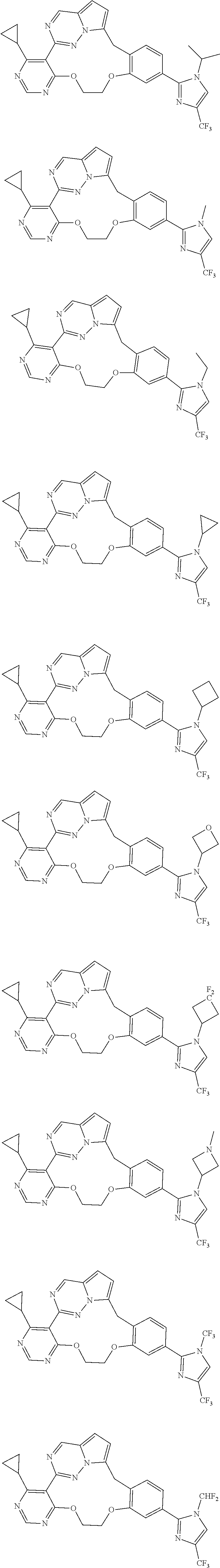

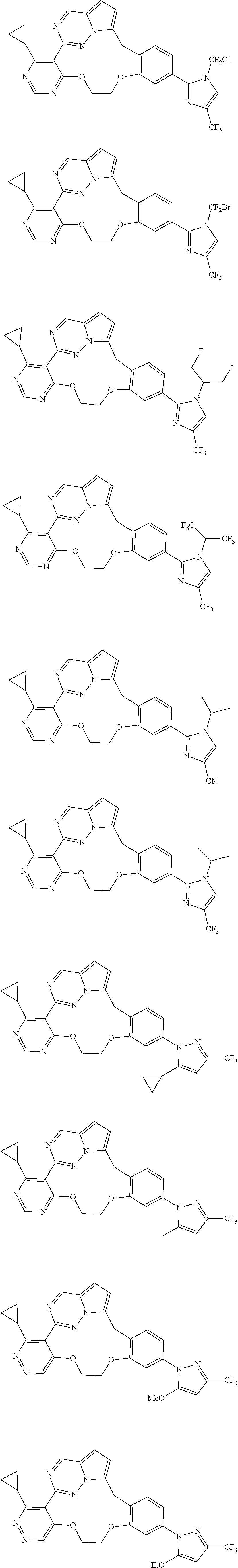

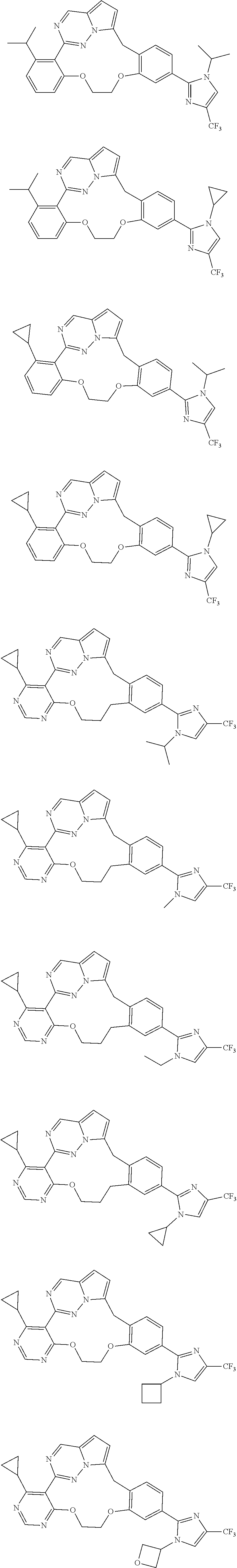

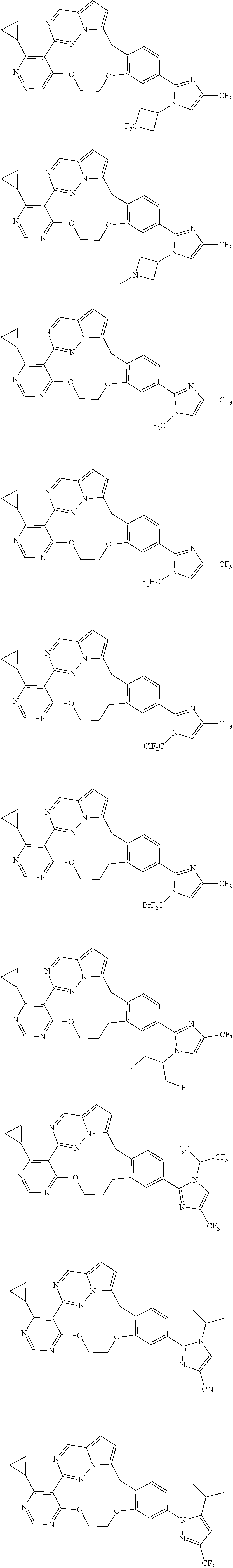

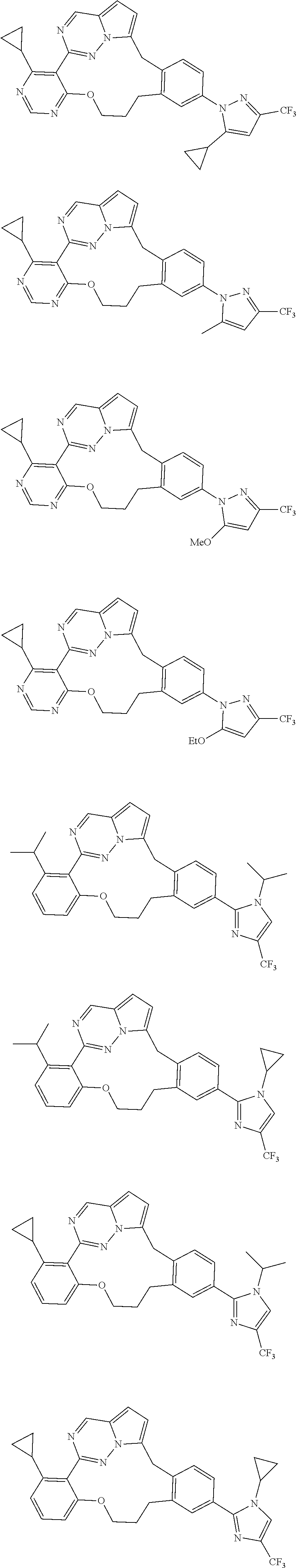

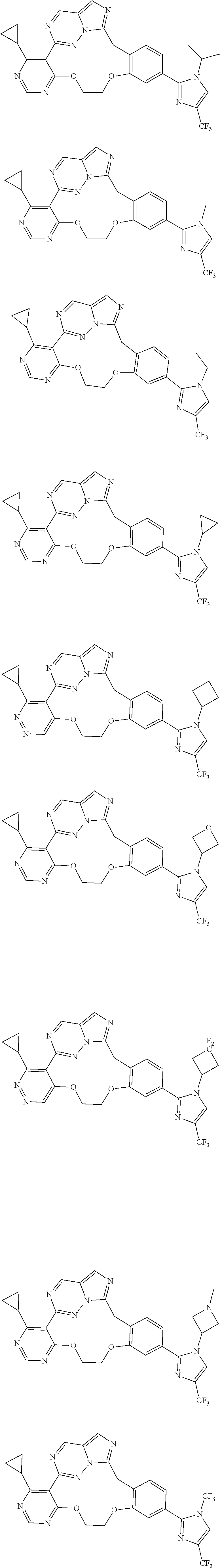

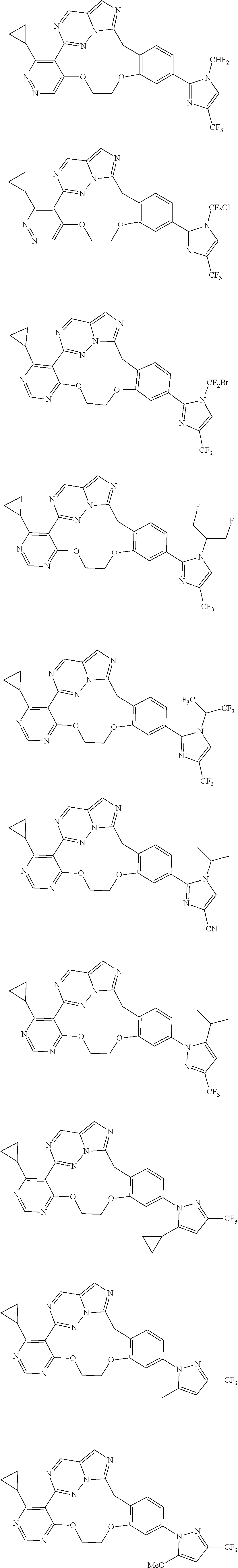

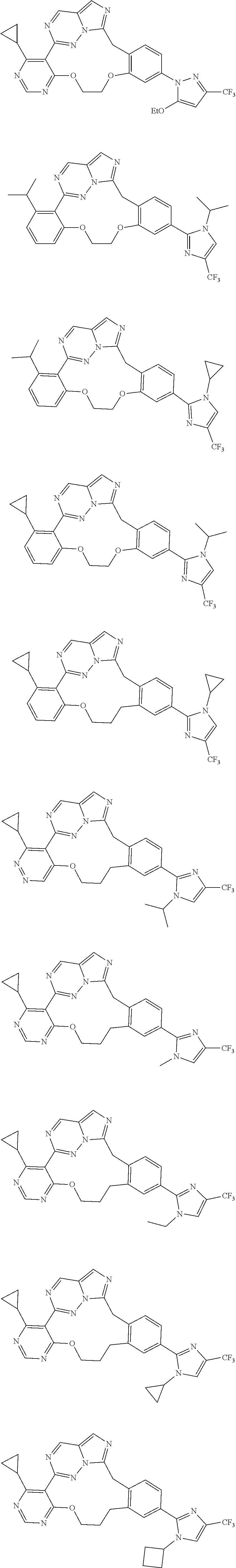

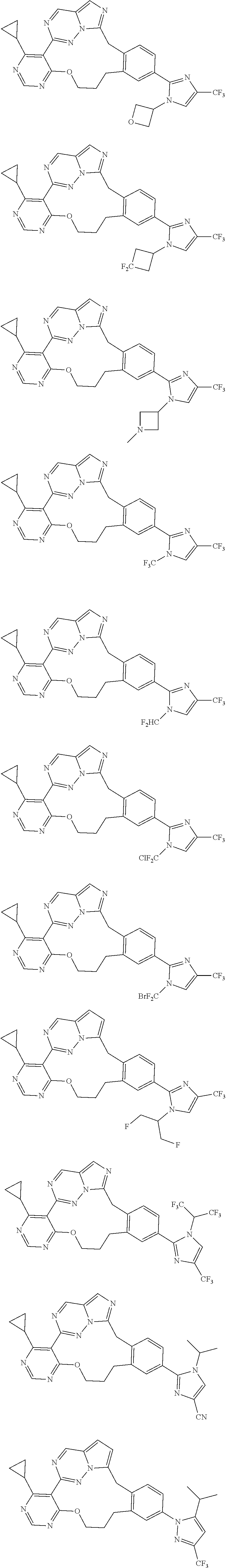

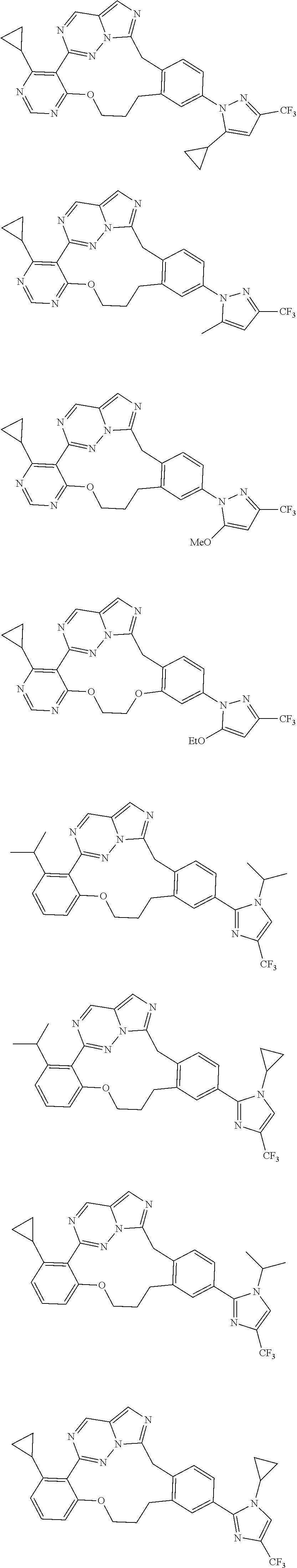

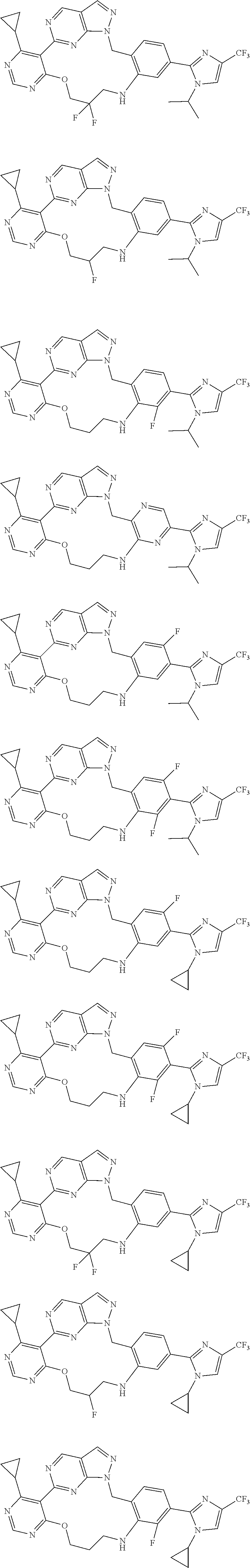

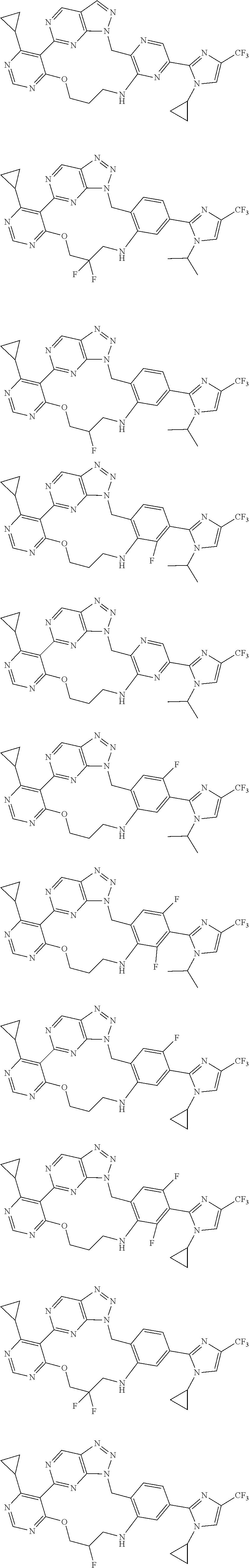

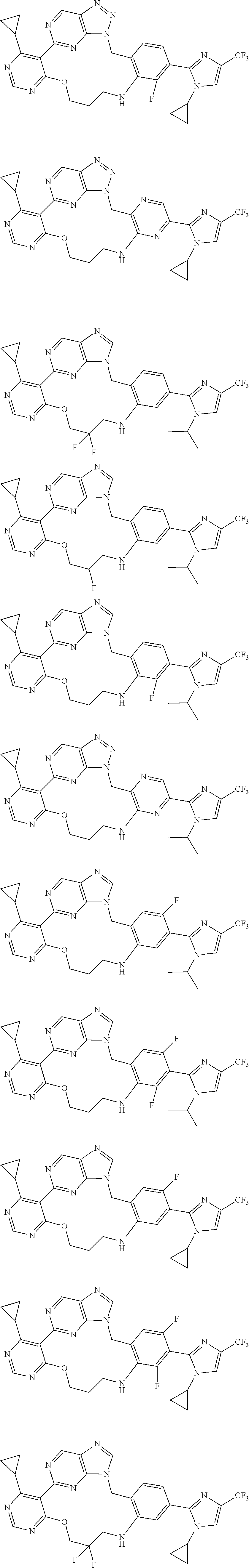

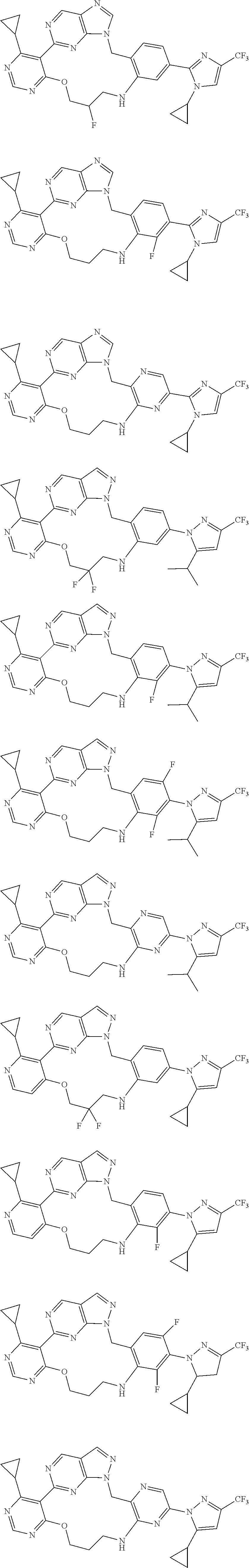

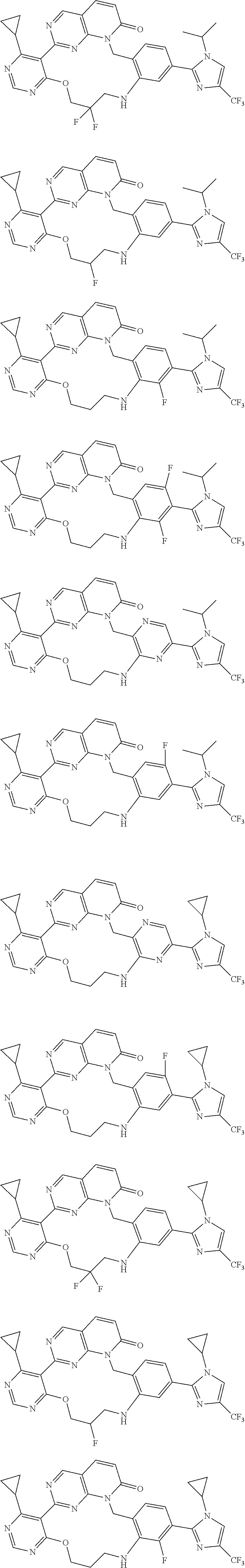

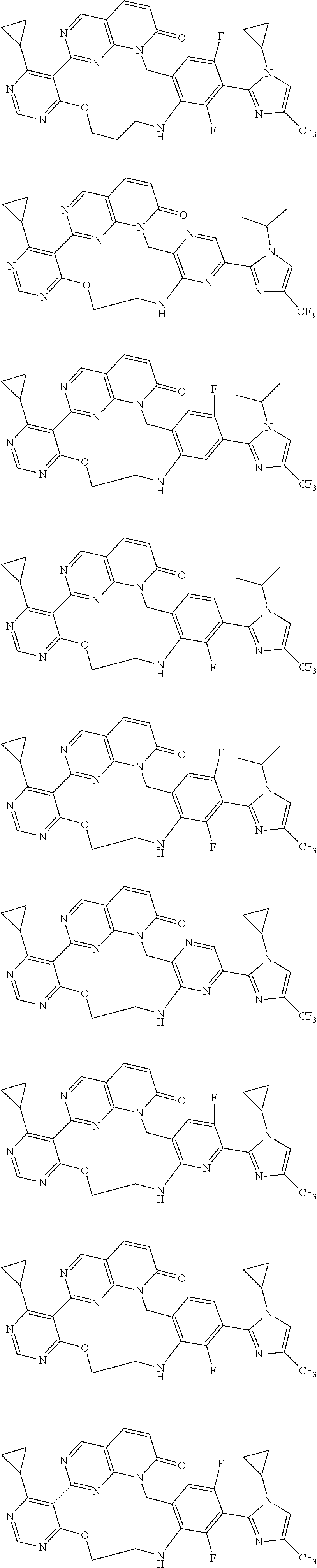

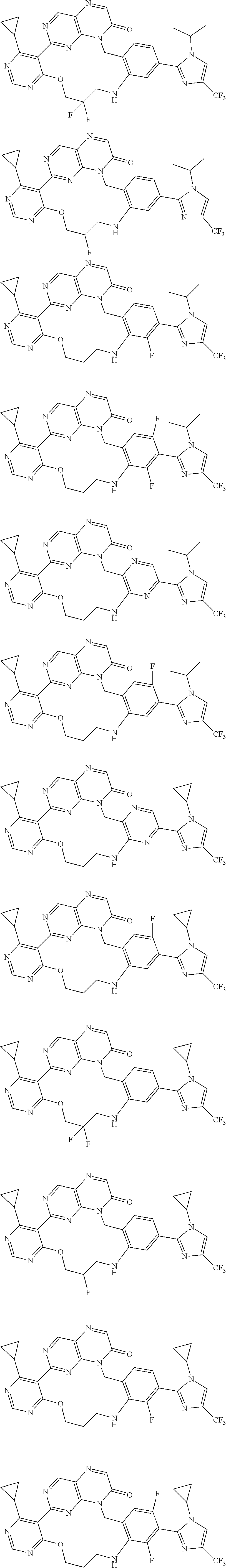

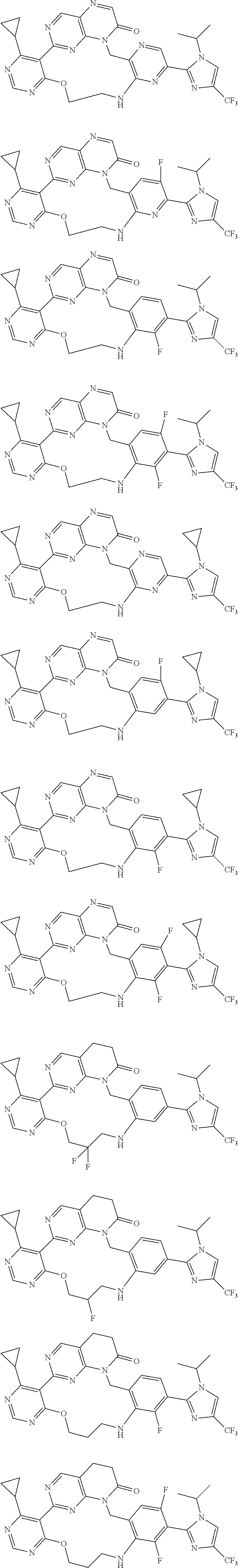

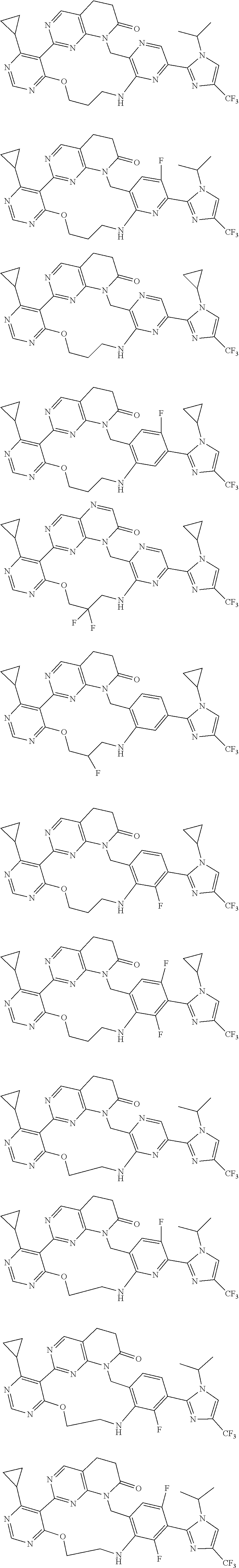

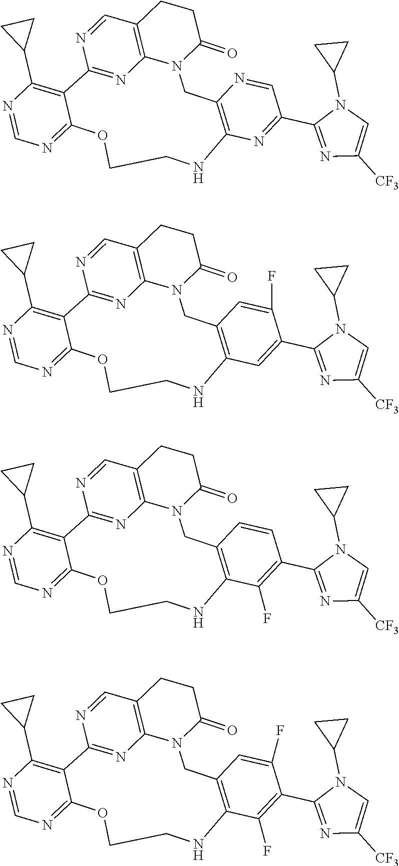 [SMILES: O=C1CCc2cnc3nc2N1Cc1ncc(-c2nc(C(F)(F)F)cn2C2CC2)nc1NCCOc1ncnc(C2CC2)c1-3 ; O=C1CCc2cnc3nc2N1Cc1cc(F)c(-c2nc(C(F)(F)F)cn2C2CC2)cc1NCCOc1ncnc(C2CC2)c1-3 ; O=C1CCc2cnc3nc2N1Cc1ccc(-c2nc(C(F)(F)F)cn2C2CC2)c(F)c1NCCOc1ncnc(C2CC2)c1-3 ; O=C1CCc2cnc3nc2N1Cc1cc(F)c(-c2nc(C(F)(F)F)cn2C2CC2)c(F)c1NCCOc1ncnc(C2CC2)c1-3]

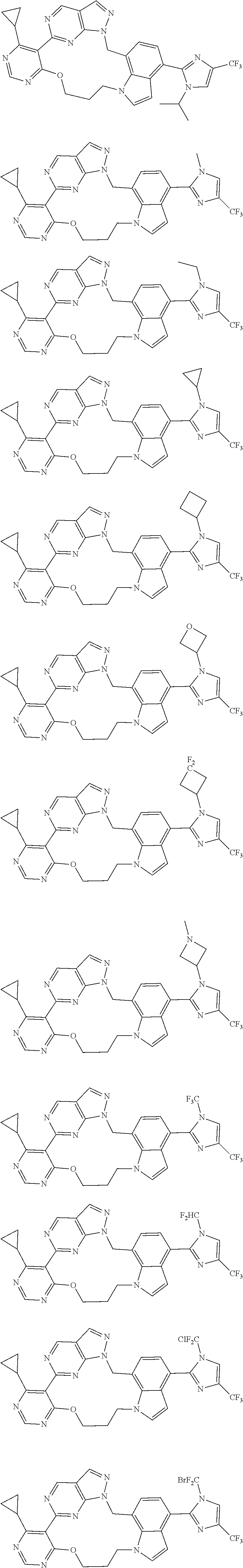

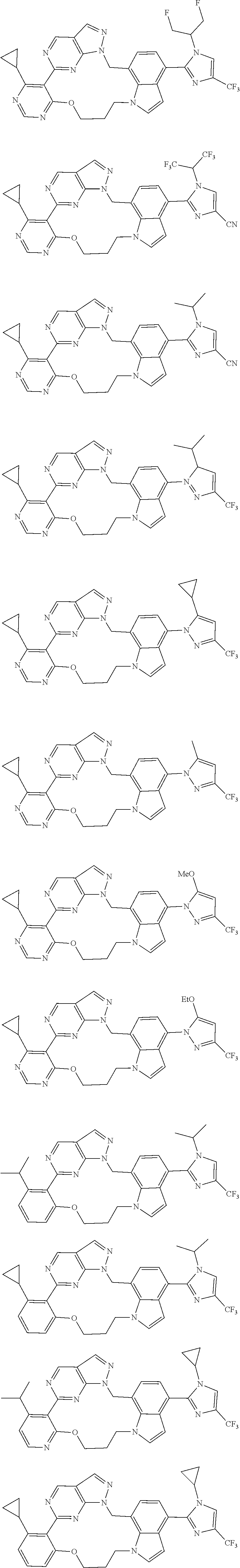

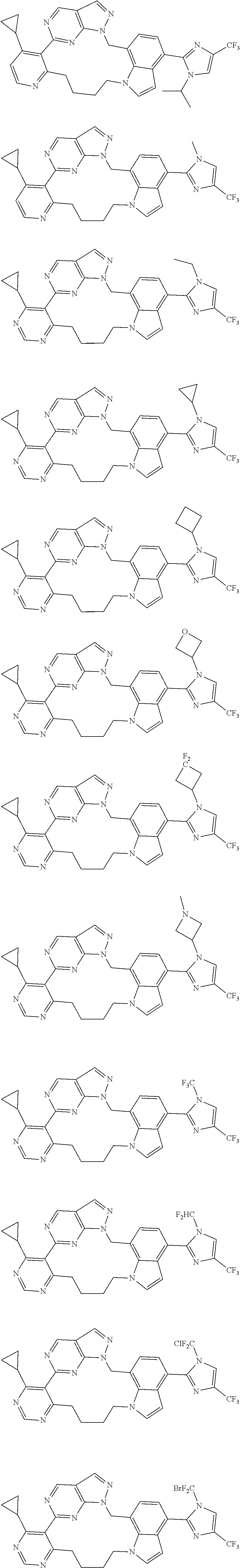

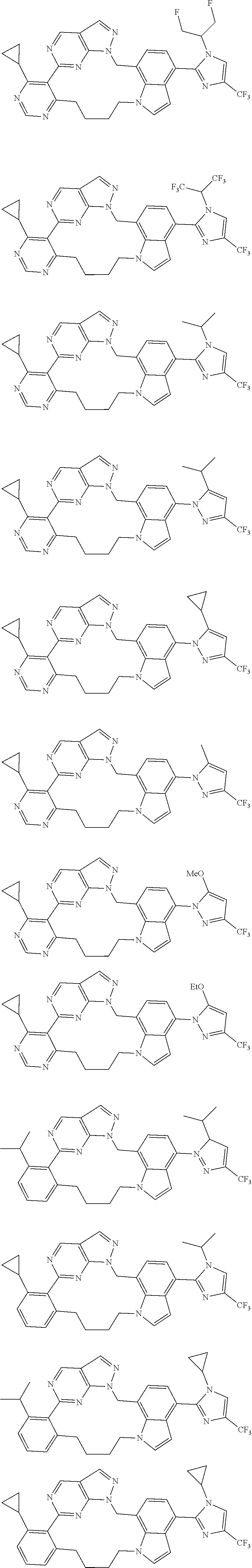

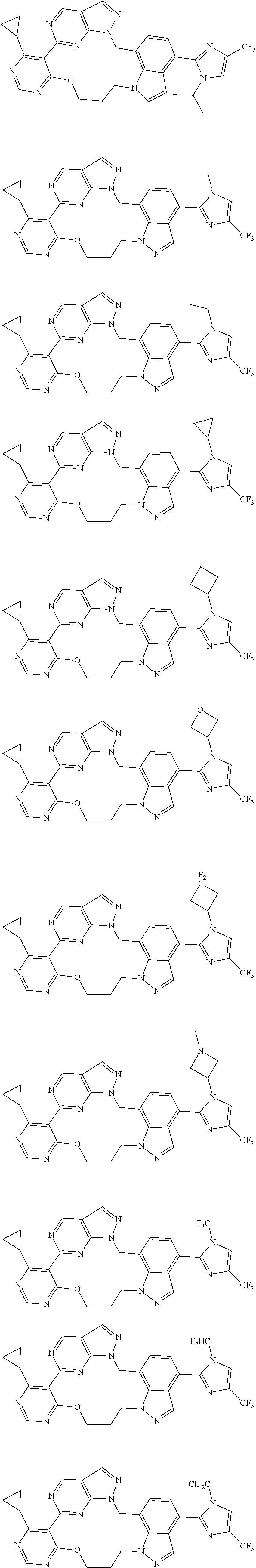

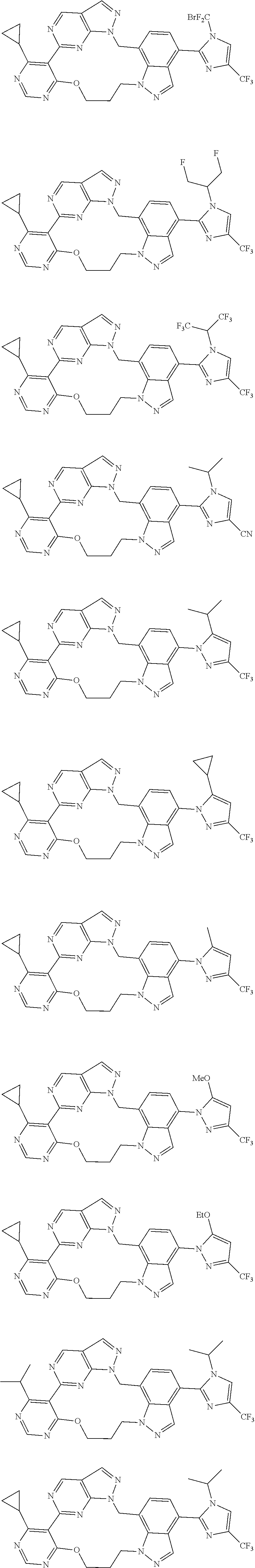

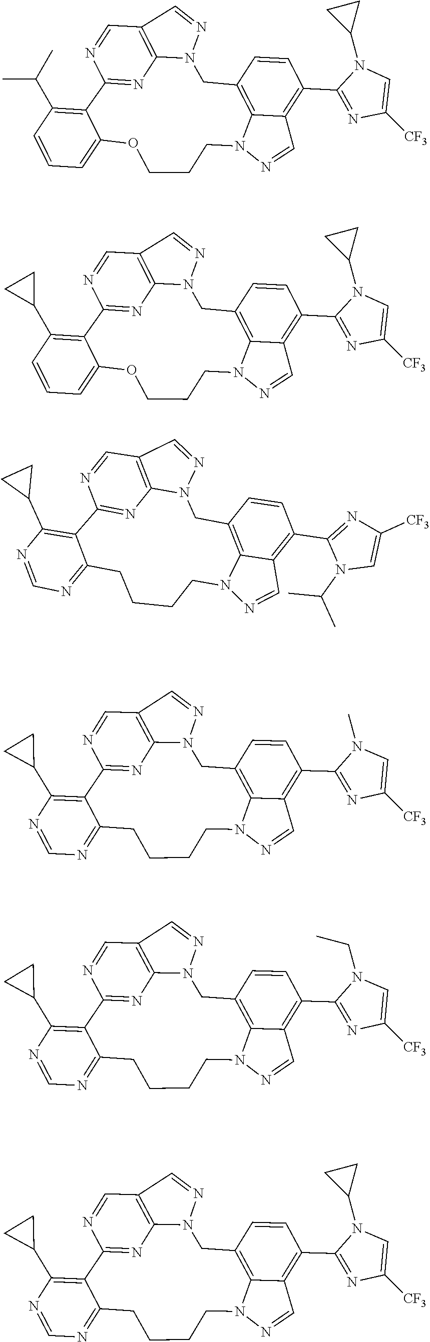

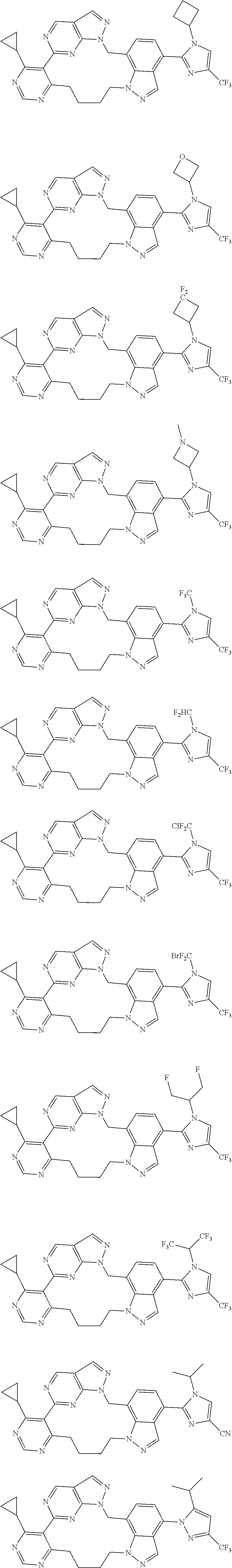

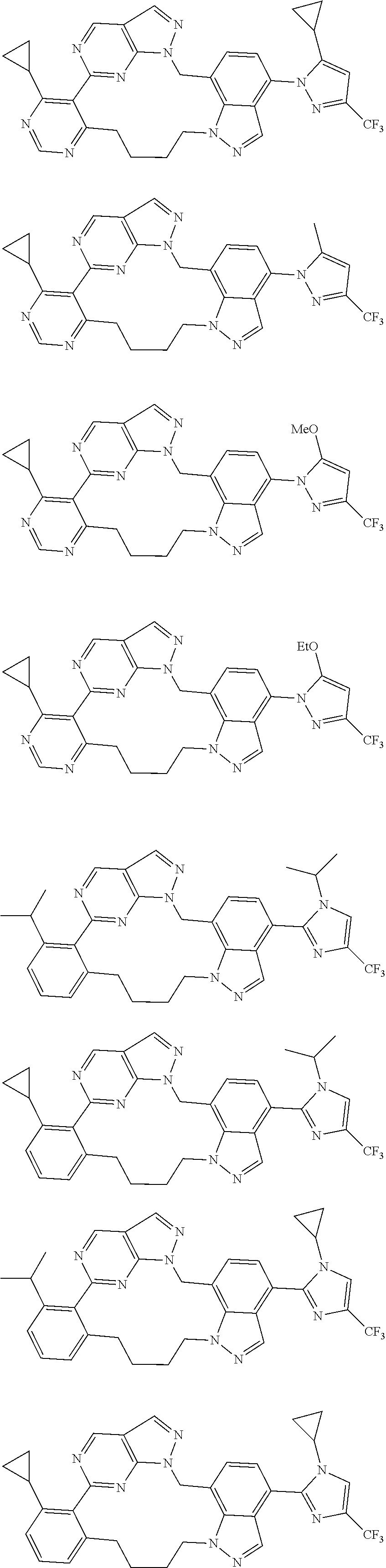

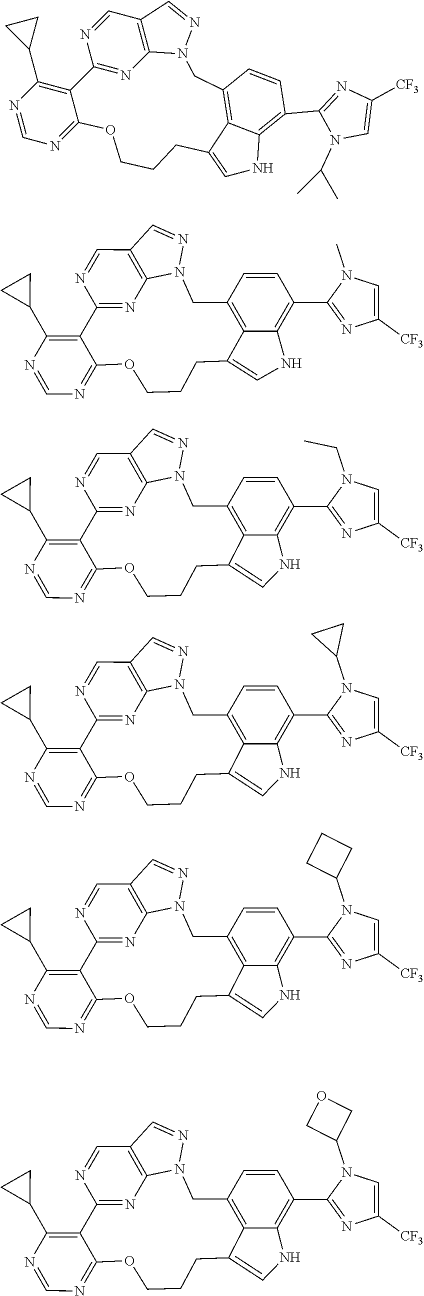

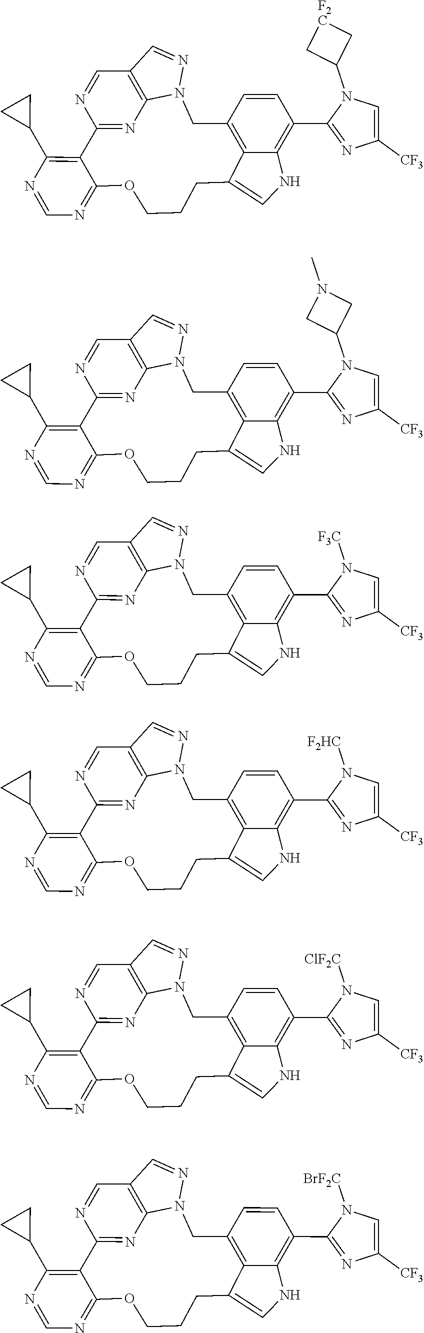

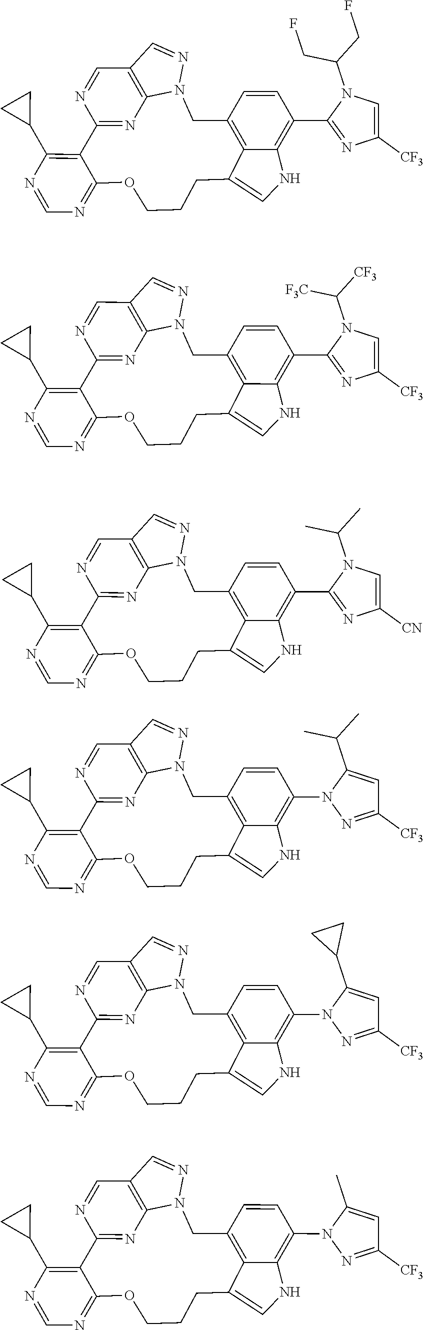

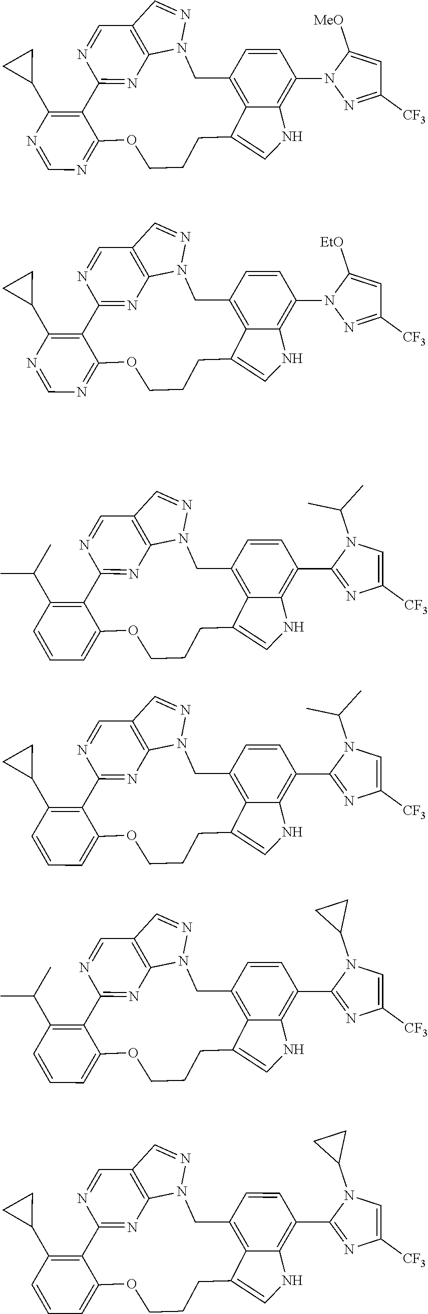

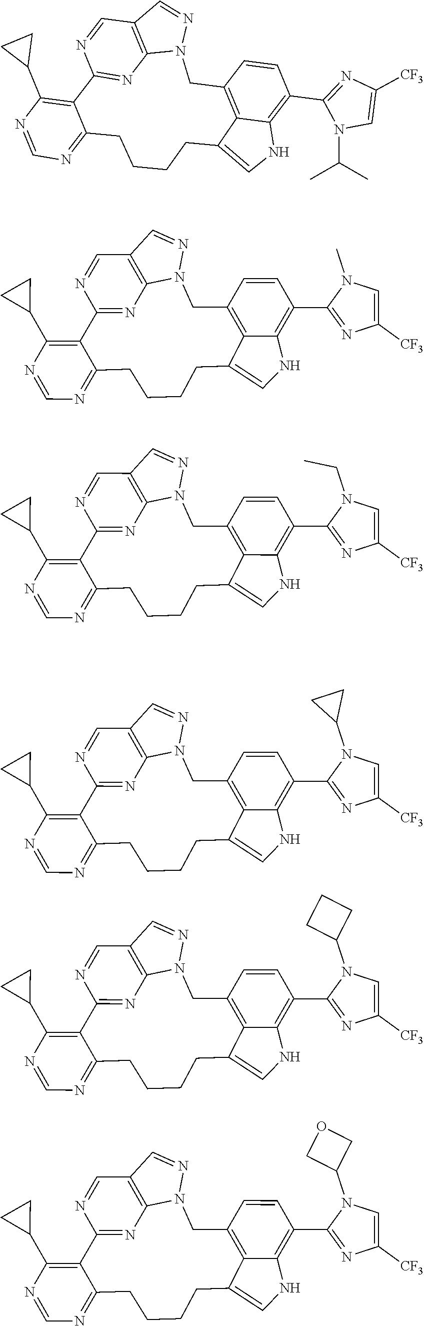 [SMILES: CC(C)n1cc(C(F)(F)F)nc1-c1ccc2c3c(c[nH]c13)CCCCc1ncnc(C3CC3)c1-c1ncc3cnn(c3n1)C2 ; Cn1cc(C(F)(F)F)nc1-c1ccc2c3c(c[nH]c13)CCCCc1ncnc(C3CC3)c1-c1ncc3cnn(c3n1)C2 ; CCn1cc(C(F)(F)F)nc1-c1ccc2c3c(c[nH]c13)CCCCc1ncnc(C3CC3)c1-c1ncc3cnn(c3n1)C2 ; FC(F)(F)c1cn(C2CC2)c(-c2ccc3c4c(c[nH]c24)CCCCc2ncnc(C4CC4)c2-c2ncc4cnn(c4n2)C3)n1 ; FC(F)(F)c1cn(C2CCC2)c(-c2ccc3c4c(c[nH]c24)CCCCc2ncnc(C4CC4)c2-c2ncc4cnn(c4n2)C3)n1 ; FC(F)(F)c1cn(C2COC2)c(-c2ccc3c4c(c[nH]c24)CCCCc2ncnc(C4CC4)c2-c2ncc4cnn(c4n2)C3)n1]

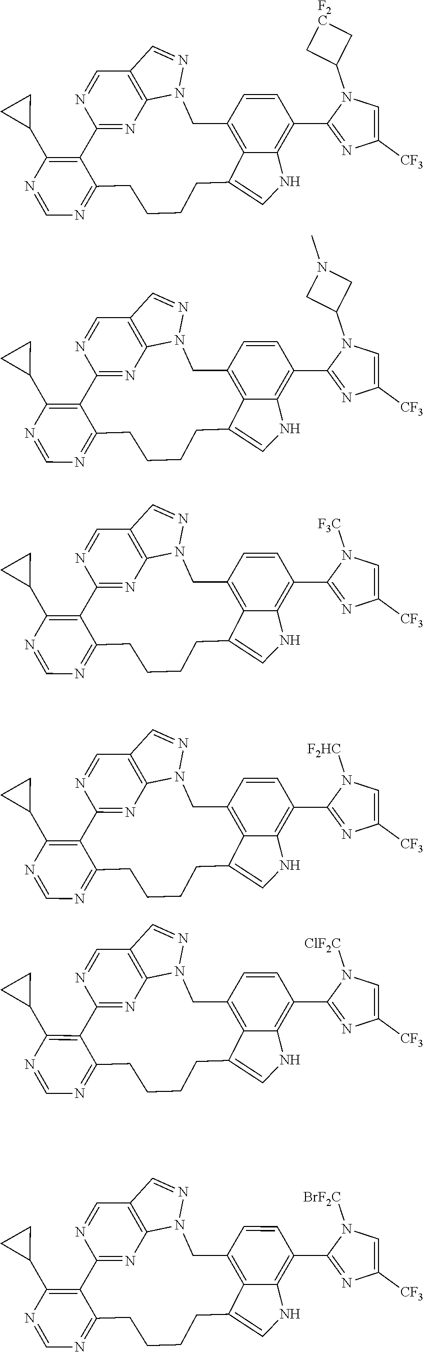

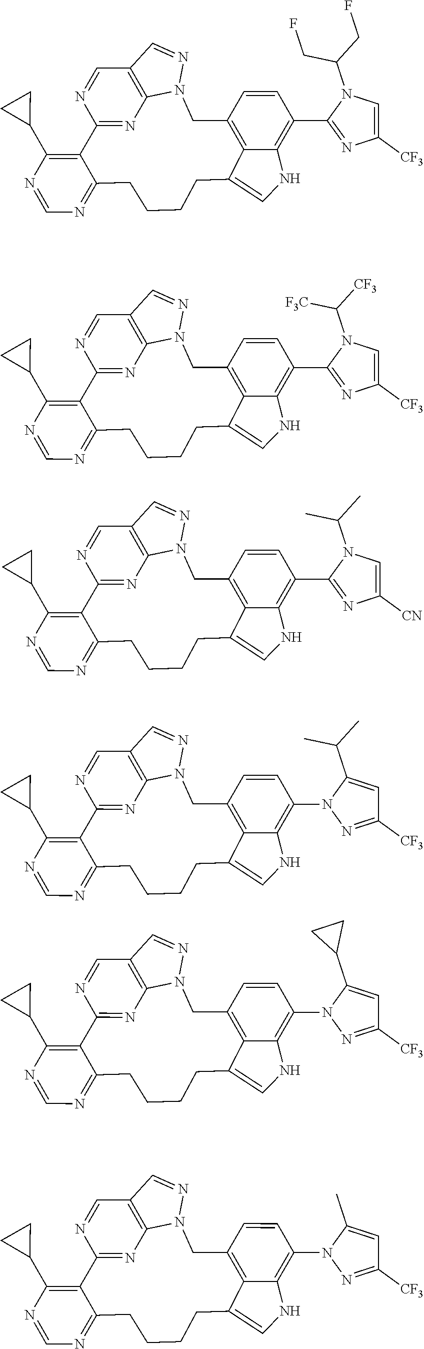

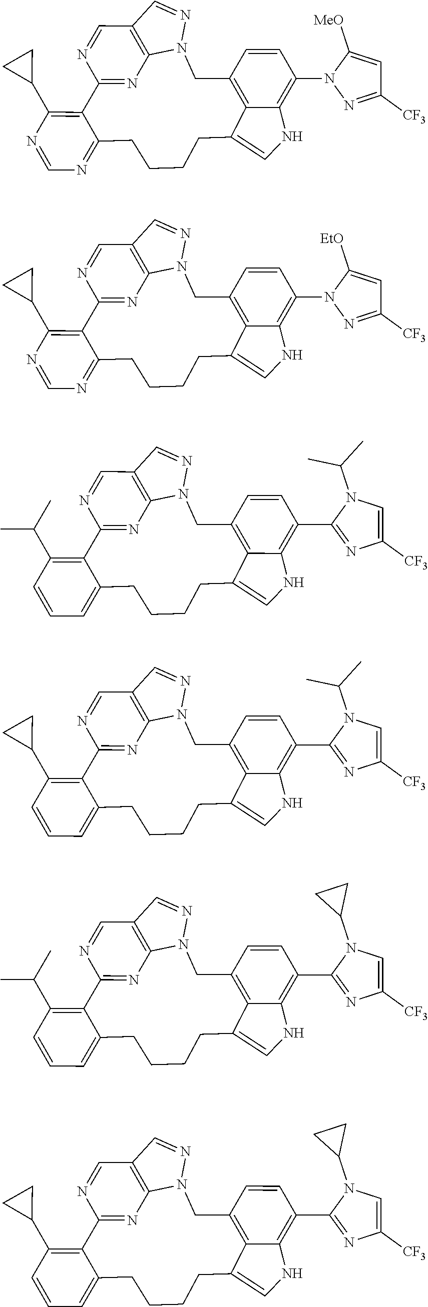 [SMILES: COc1cc(C(F)(F)F)nn1-c1ccc2c3c(c[nH]c13)CCCCc1ncnc(C3CC3)c1-c1ncc3cnn(c3n1)C2 ; CCOc1cc(C(F)(F)F)nn1-c1ccc2c3c(c[nH]c13)CCCCc1ncnc(C3CC3)c1-c1ncc3cnn(c3n1)C2 ; CC(C)c1cccc2c1-c1ncc3cnn(c3n1)Cc1ccc(-c3nc(C(F)(F)F)cn3C(C)C)c3[nH]cc(c13)CCCC2 ; CC(C)n1cc(C(F)(F)F)nc1-c1ccc2c3c(c[nH]c13)CCCCc1cccc(C3CC3)c1-c1ncc3cnn(c3n1)C2 ; CC(C)c1cccc2c1-c1ncc3cnn(c3n1)Cc1ccc(-c3nc(C(F)(F)F)cn3C3CC3)c3[nH]cc(c13)CCCC2 ; FC(F)(F)c1cn(C2CC2)c(-c2ccc3c4c(c[nH]c24)CCCCc2cccc(C4CC4)c2-c2ncc4cnn(c4n2)C3)n1]

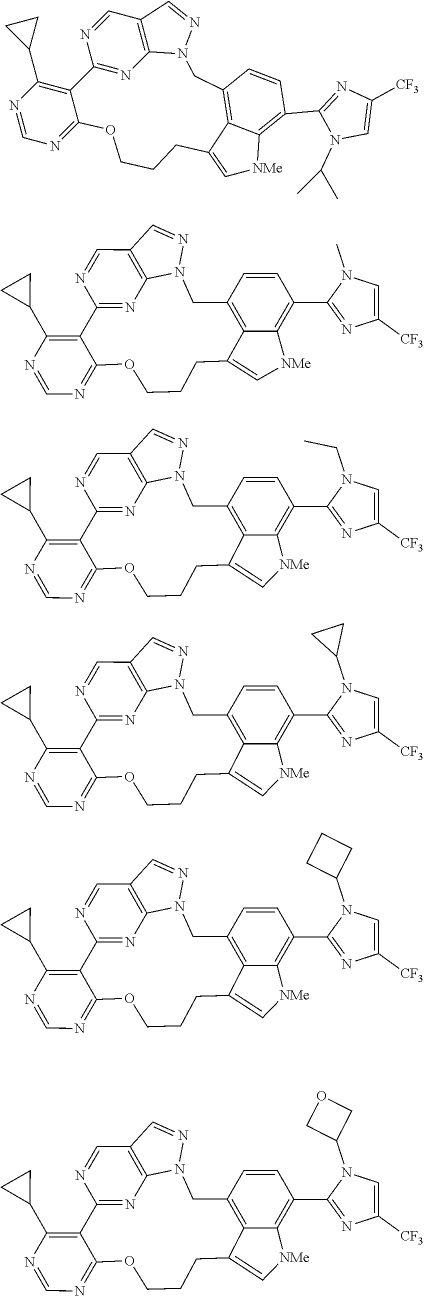

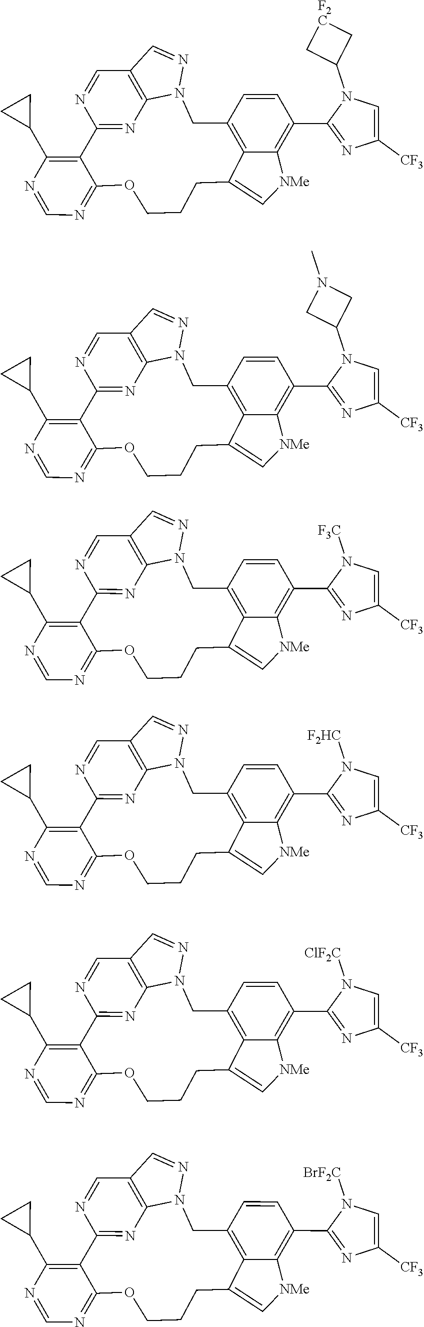

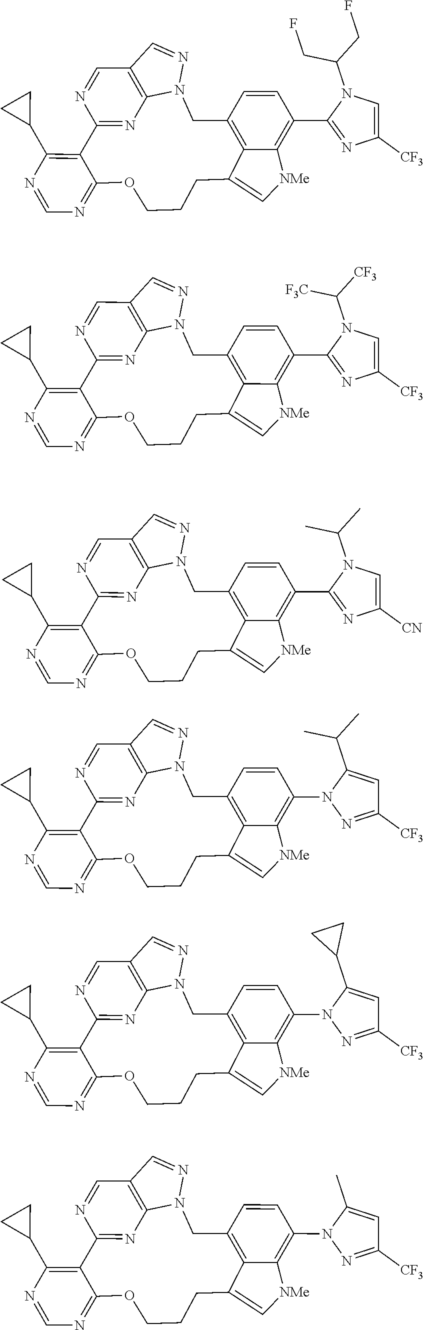

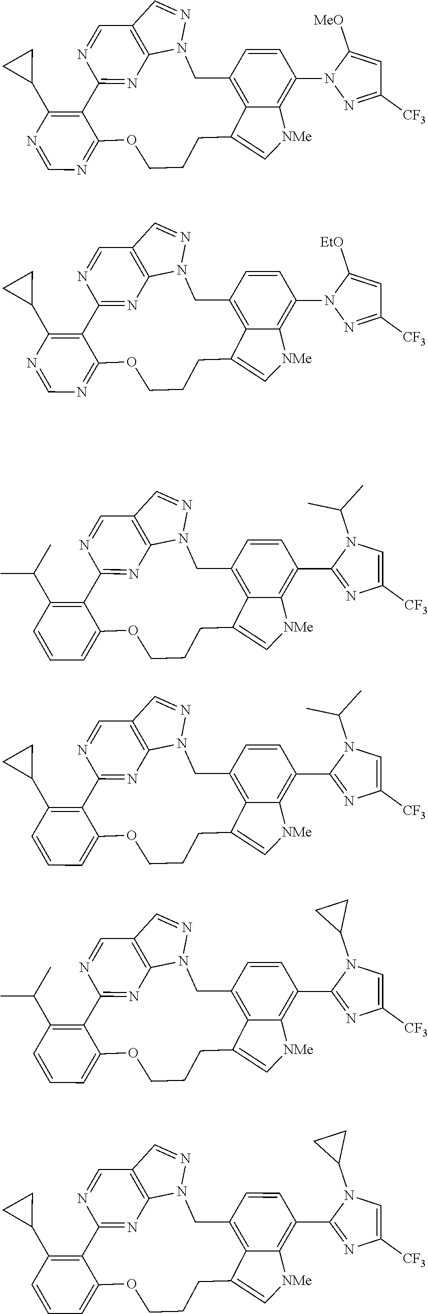

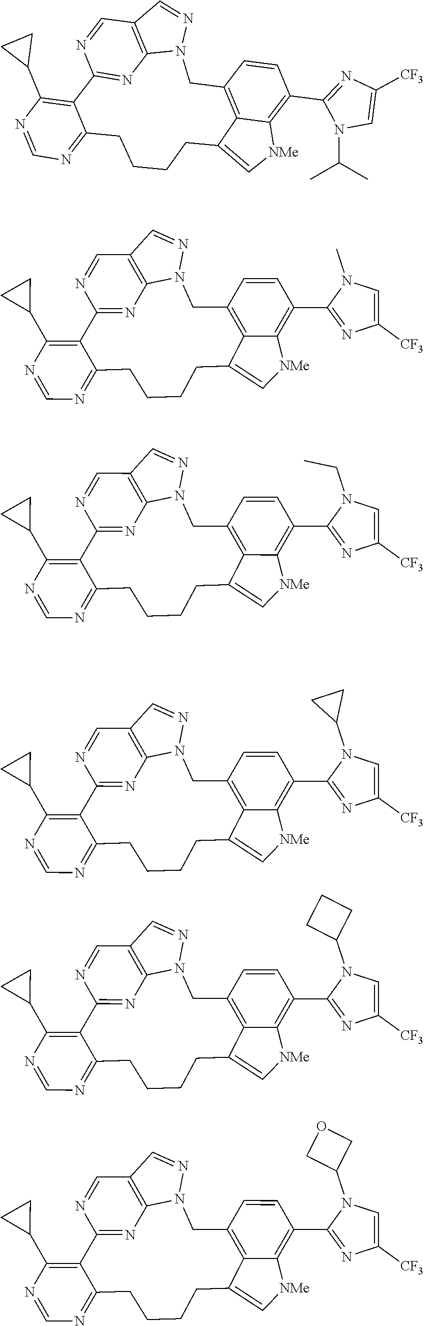 [SMILES: CC(C)n1cc(C(F)(F)F)nc1-c1ccc2c3c(cn(C)c13)CCCCc1ncnc(C3CC3)c1-c1ncc3cnn(c3n1)C2 ; Cn1cc(C(F)(F)F)nc1-c1ccc2c3c(cn(C)c13)CCCCc1ncnc(C3CC3)c1-c1ncc3cnn(c3n1)C2 ; CCn1cc(C(F)(F)F)nc1-c1ccc2c3c(cn(C)c13)CCCCc1ncnc(C3CC3)c1-c1ncc3cnn(c3n1)C2 ; Cn1cc2c3c(ccc(-c4nc(C(F)(F)F)cn4C4CC4)c31)Cn1ncc3cnc(nc31)-c1c(ncnc1C1CC1)CCCC2 ; Cn1cc2c3c(ccc(-c4nc(C(F)(F)F)cn4C4CCC4)c31)Cn1ncc3cnc(nc31)-c1c(ncnc1C1CC1)CCCC2 ; Cn1cc2c3c(ccc(-c4nc(C(F)(F)F)cn4C4COC4)c31)Cn1ncc3cnc(nc31)-c1c(ncnc1C1CC1)CCCC2]

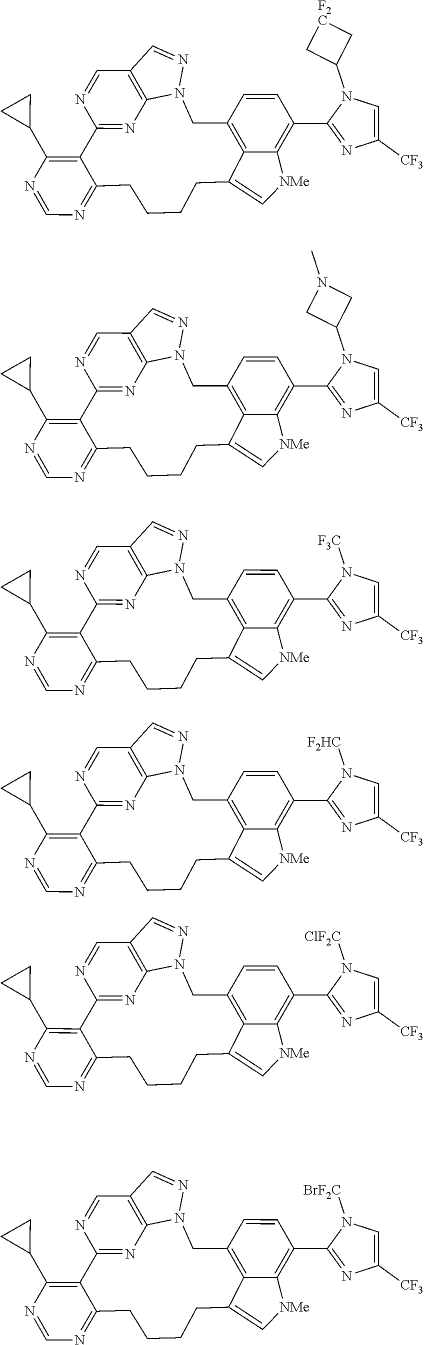

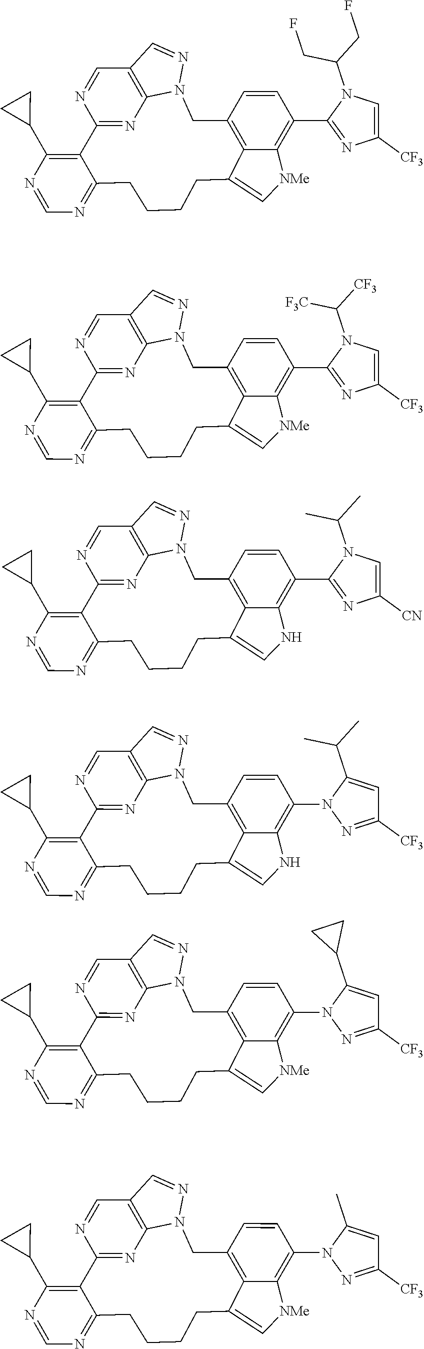

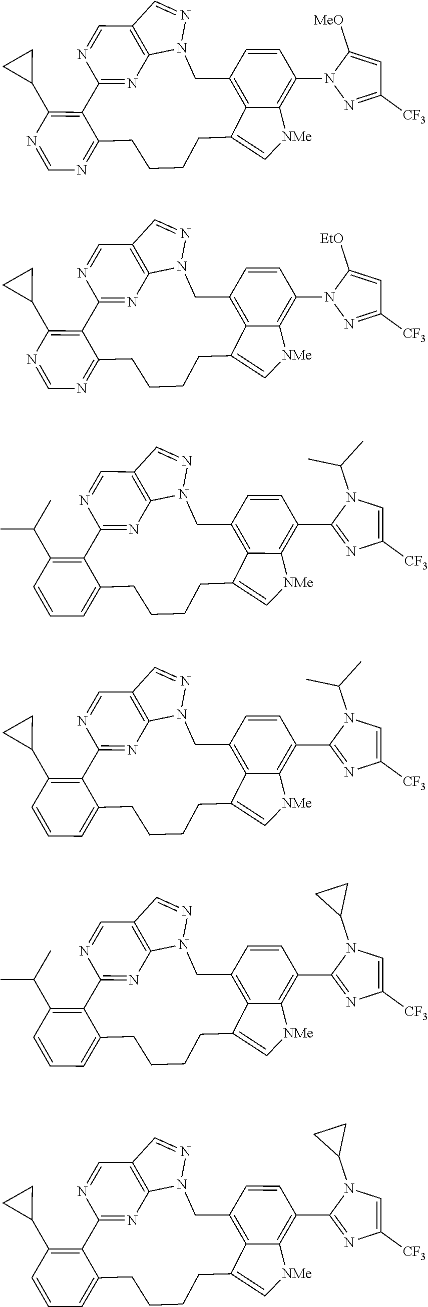 [SMILES: COc1cc(C(F)(F)F)nn1-c1ccc2c3c(cn(C)c13)CCCCc1ncnc(C3CC3)c1-c1ncc3cnn(c3n1)C2 ; CCOc1cc(C(F)(F)F)nn1-c1ccc2c3c(cn(C)c13)CCCCc1ncnc(C3CC3)c1-c1ncc3cnn(c3n1)C2 ; CC(C)c1cccc2c1-c1ncc3cnn(c3n1)Cc1ccc(-c3nc(C(F)(F)F)cn3C(C)C)c3c1c(cn3C)CCCC2 ; CC(C)n1cc(C(F)(F)F)nc1-c1ccc2c3c(cn(C)c13)CCCCc1cccc(C3CC3)c1-c1ncc3cnn(c3n1)C2 ; CC(C)c1cccc2c1-c1ncc3cnn(c3n1)Cc1ccc(-c3nc(C(F)(F)F)cn3C3CC3)c3c1c(cn3C)CCCC2 ; Cn1cc2c3c(ccc(-c4nc(C(F)(F)F)cn4C4CC4)c31)Cn1ncc3cnc(nc31)-c1c(cccc1C1CC1)CCCC2]

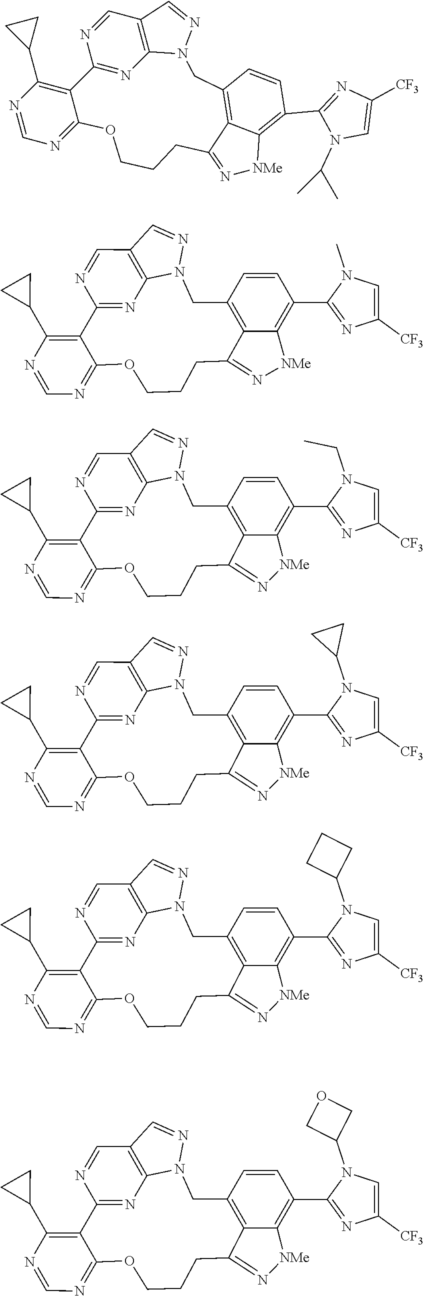

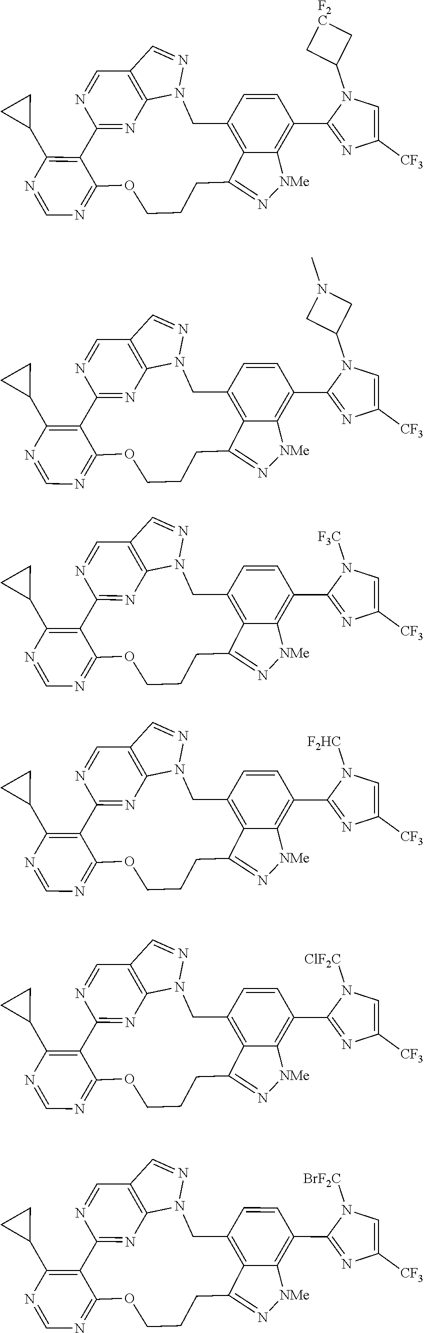

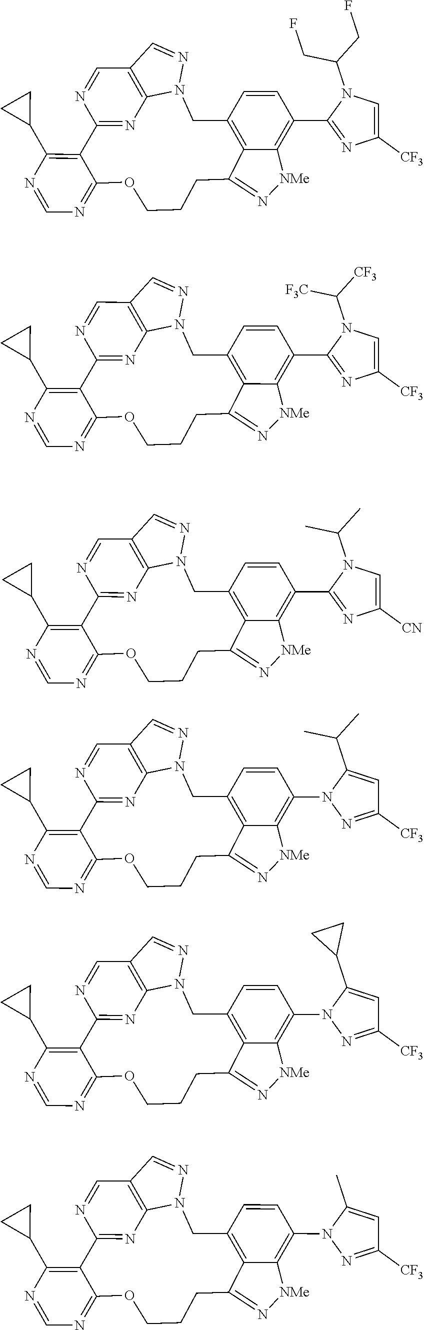

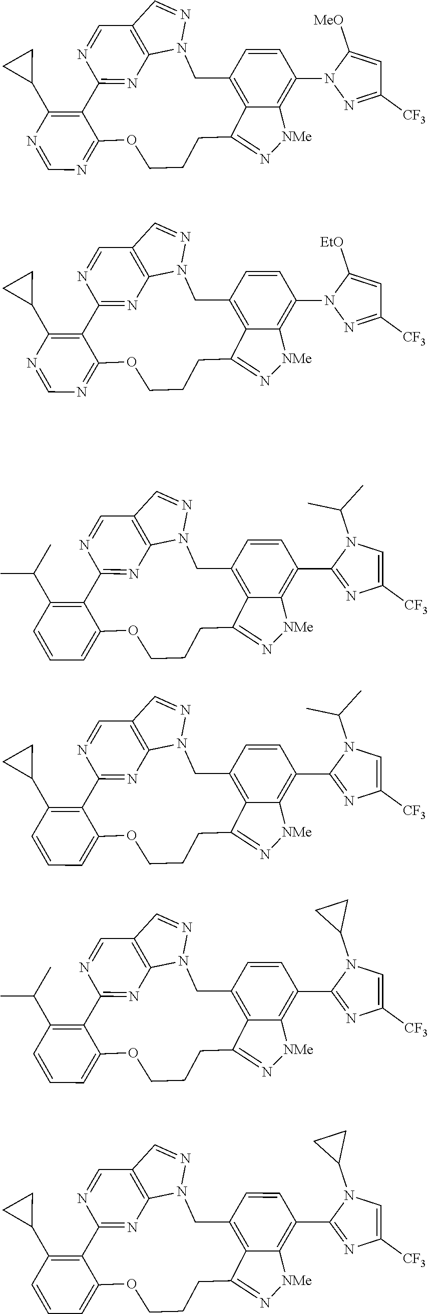

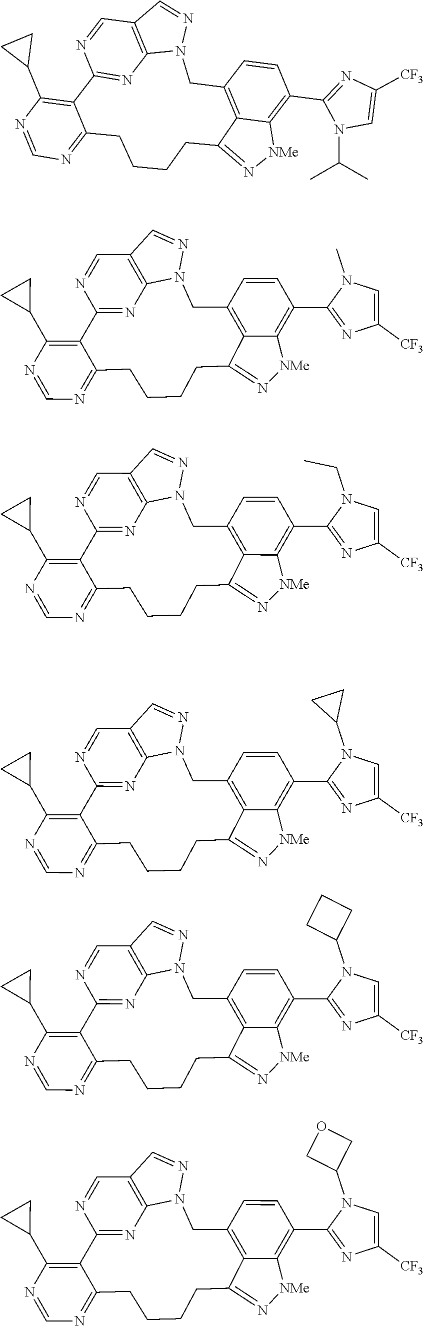 [SMILES: CC(C)n1cc(C(F)(F)F)nc1-c1ccc2c3c(nn(C)c13)CCCCc1ncnc(C3CC3)c1-c1ncc3cnn(c3n1)C2 ; Cn1cc(C(F)(F)F)nc1-c1ccc2c3c(nn(C)c13)CCCCc1ncnc(C3CC3)c1-c1ncc3cnn(c3n1)C2 ; CCn1cc(C(F)(F)F)nc1-c1ccc2c3c(nn(C)c13)CCCCc1ncnc(C3CC3)c1-c1ncc3cnn(c3n1)C2 ; Cn1nc2c3c(ccc(-c4nc(C(F)(F)F)cn4C4CC4)c31)Cn1ncc3cnc(nc31)-c1c(ncnc1C1CC1)CCCC2 ; Cn1nc2c3c(ccc(-c4nc(C(F)(F)F)cn4C4CCC4)c31)Cn1ncc3cnc(nc31)-c1c(ncnc1C1CC1)CCCC2 ; Cn1nc2c3c(ccc(-c4nc(C(F)(F)F)cn4C4COC4)c31)Cn1ncc3cnc(nc31)-c1c(ncnc1C1CC1)CCCC2]

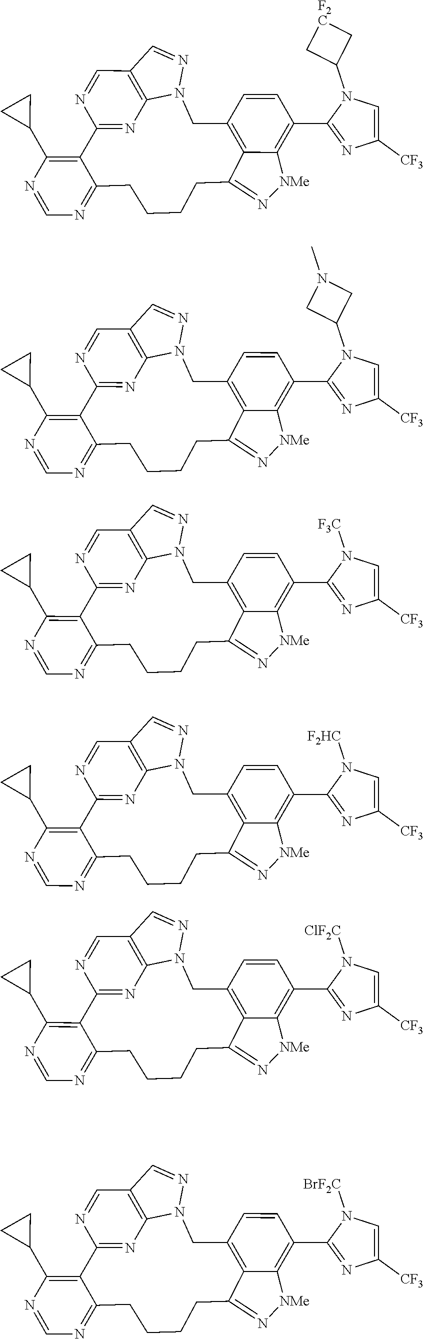

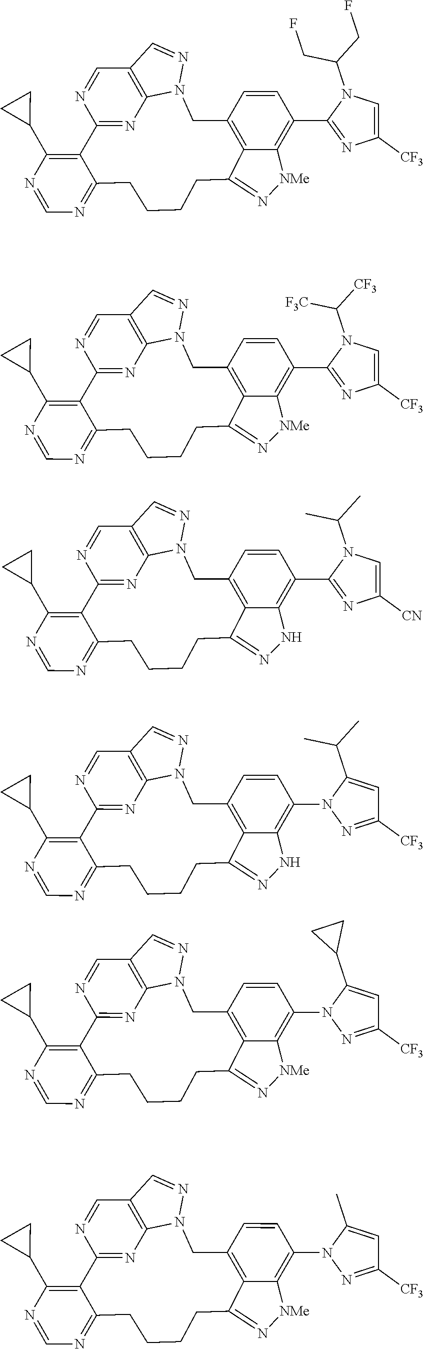

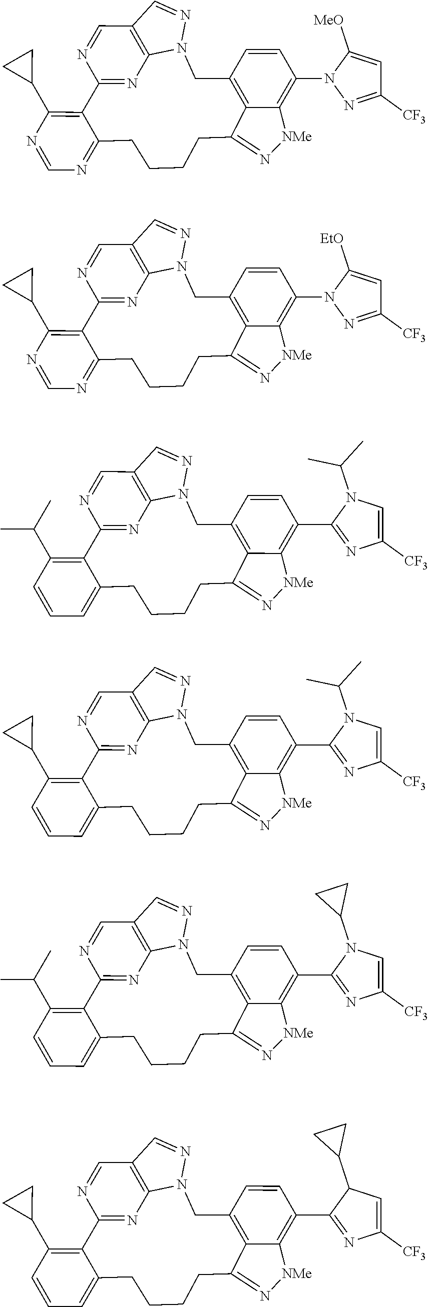 [SMILES: COc1cc(C(F)(F)F)nn1-c1ccc2c3c(nn(C)c13)CCCCc1ncnc(C3CC3)c1-c1ncc3cnn(c3n1)C2 ; CCOc1cc(C(F)(F)F)nn1-c1ccc2c3c(nn(C)c13)CCCCc1ncnc(C3CC3)c1-c1ncc3cnn(c3n1)C2 ; CC(C)c1cccc2c1-c1ncc3cnn(c3n1)Cc1ccc(-c3nc(C(F)(F)F)cn3C(C)C)c3c1c(nn3C)CCCC2 ; CC(C)n1cc(C(F)(F)F)nc1-c1ccc2c3c(nn(C)c13)CCCCc1cccc(C3CC3)c1-c1ncc3cnn(c3n1)C2 ; CC(C)c1cccc2c1-c1ncc3cnn(c3n1)Cc1ccc(-c3nc(C(F)(F)F)cn3C3CC3)c3c1c(nn3C)CCCC2 ; Cn1nc2c3c(ccc(C4=NC(C(F)(F)F)=CC4C4CC4)c31)Cn1ncc3cnc(nc31)-c1c(cccc1C1CC1)CCCC2]

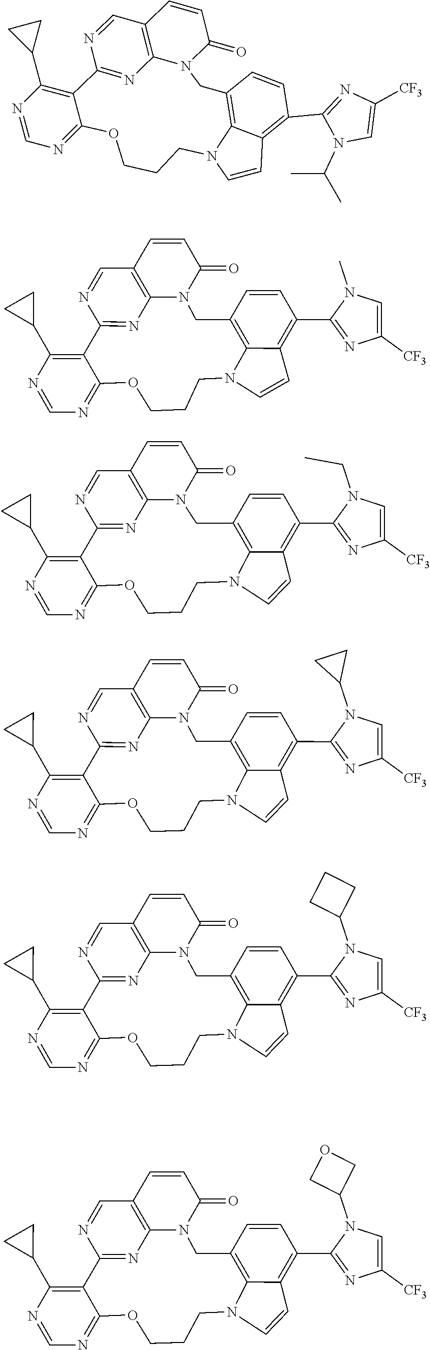

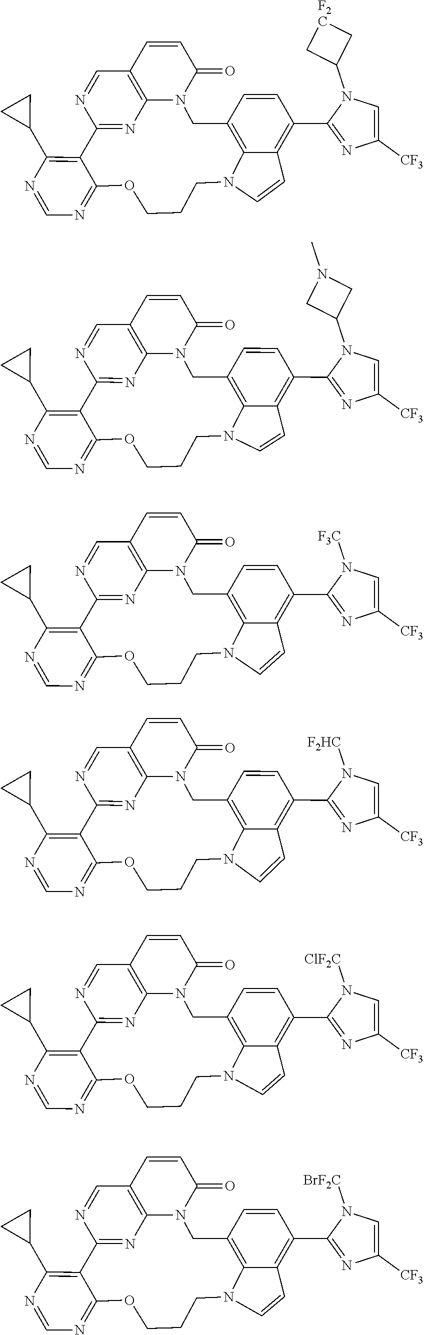

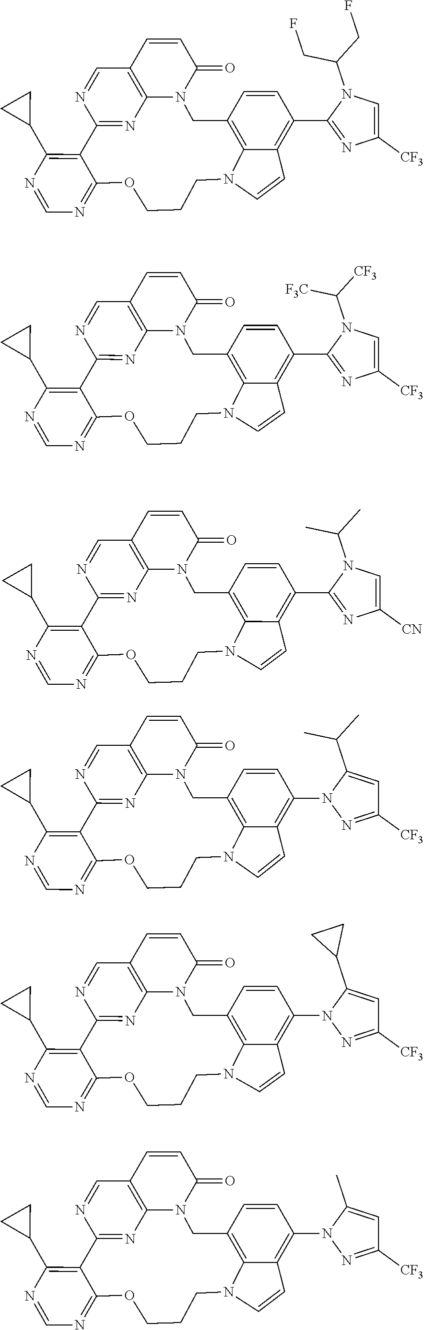

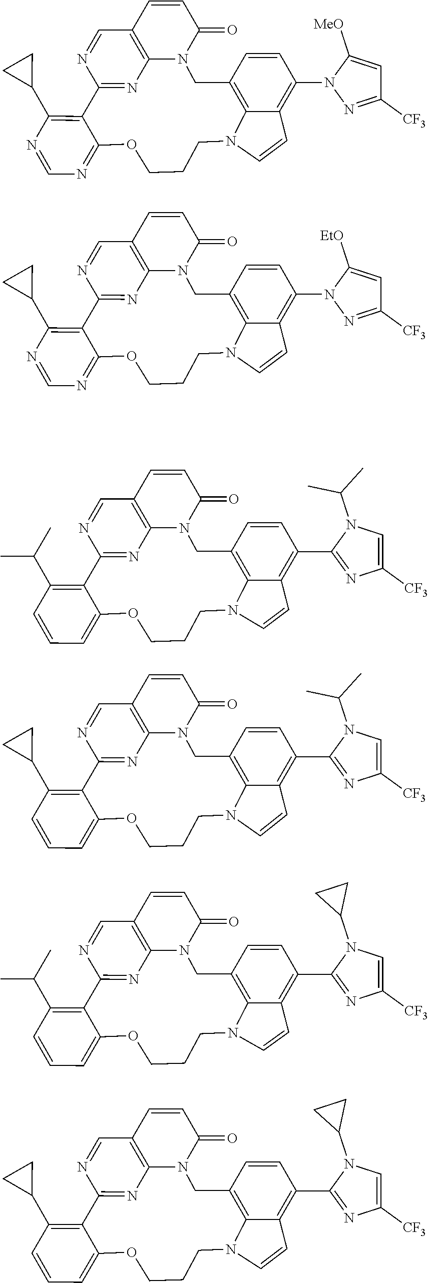

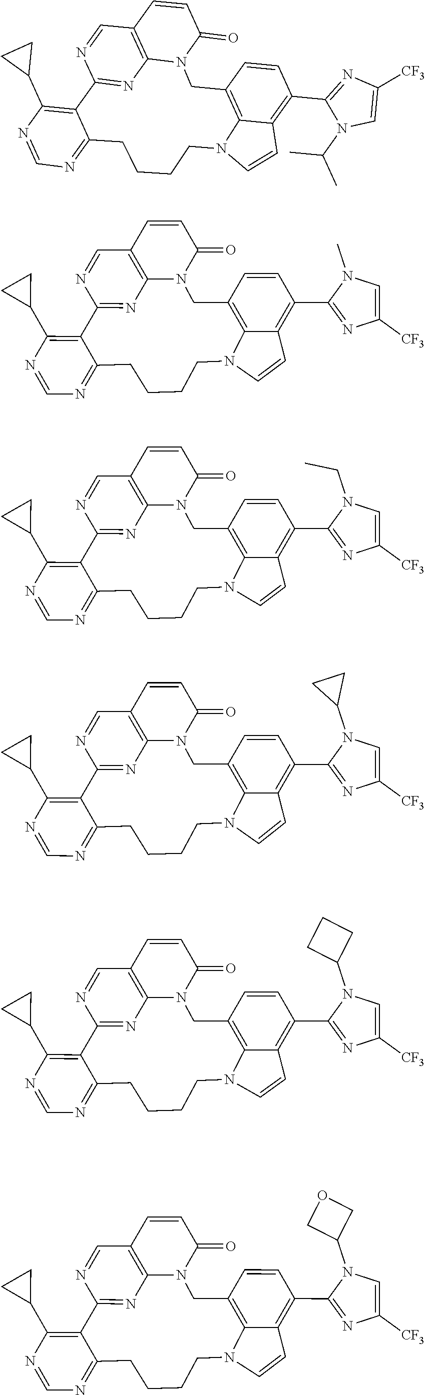 [SMILES: Cc1ccc(-c2nc(C(F)(F)F)cn2C(C)C)c2ccn(CCCCc3ncnc(C4CC4)c3-c3ncc4ccc(=O)n(C)c4n3)c12 ; Cc1ccc(-c2nc(C(F)(F)F)cn2C)c2ccn(CCCCc3ncnc(C4CC4)c3-c3ncc4ccc(=O)n(C)c4n3)c12 ; CCn1cc(C(F)(F)F)nc1-c1ccc(C)c2c1ccn2CCCCc1ncnc(C2CC2)c1-c1ncc2ccc(=O)n(C)c2n1 ; Cc1ccc(-c2nc(C(F)(F)F)cn2C2CC2)c2ccn(CCCCc3ncnc(C4CC4)c3-c3ncc4ccc(=O)n(C)c4n3)c12 ; Cc1ccc(-c2nc(C(F)(F)F)cn2C2CCC2)c2ccn(CCCCc3ncnc(C4CC4)c3-c3ncc4ccc(=O)n(C)c4n3)c12 ; Cc1ccc(-c2nc(C(F)(F)F)cn2C2COC2)c2ccn(CCCCc3ncnc(C4CC4)c3-c3ncc4ccc(=O)n(C)c4n3)c12]

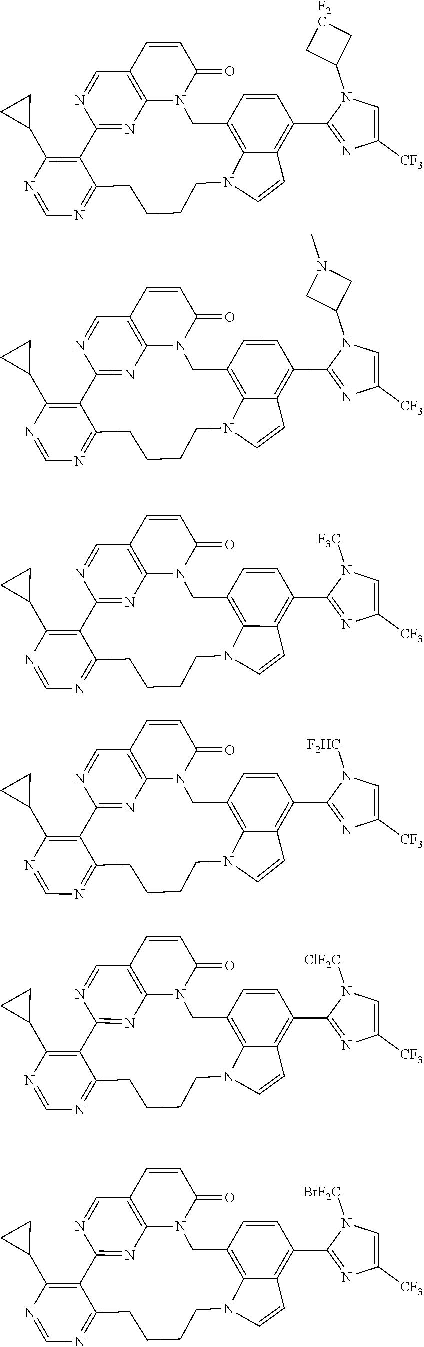

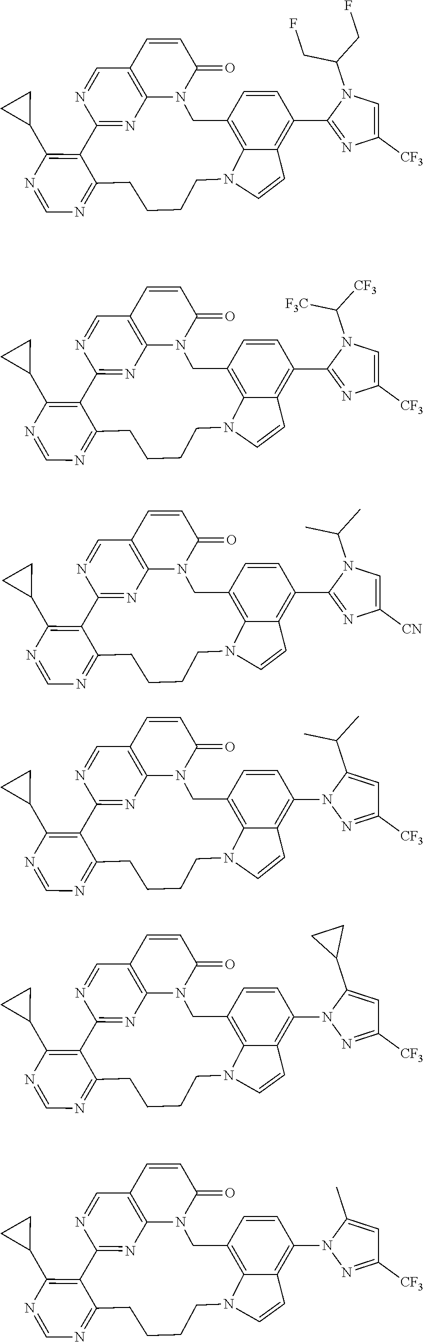

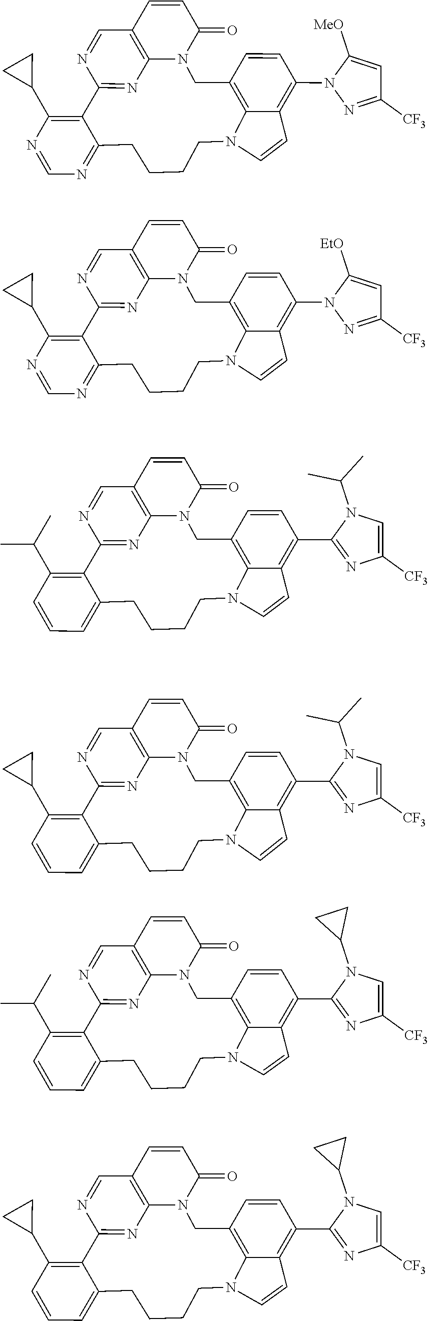

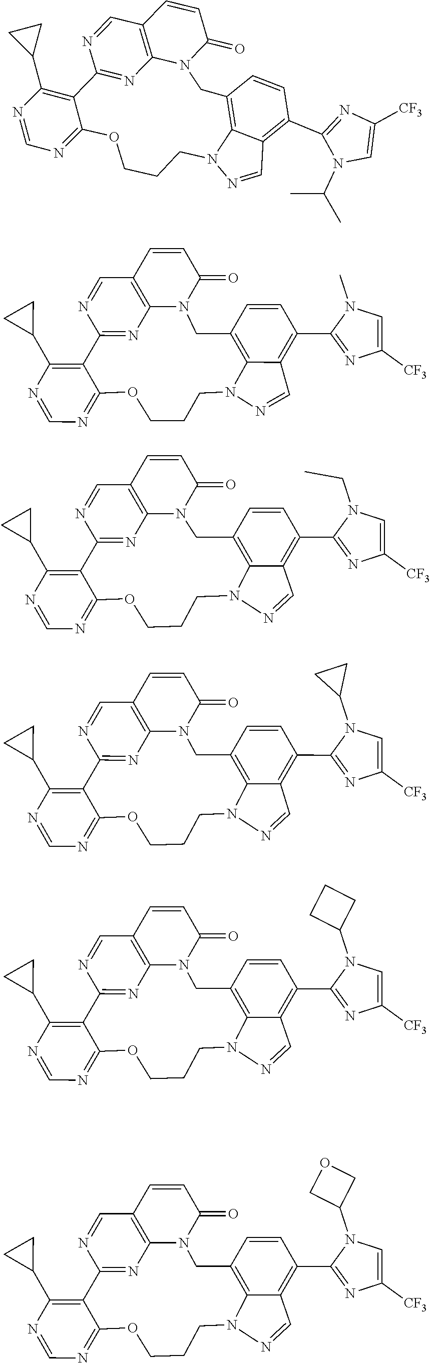

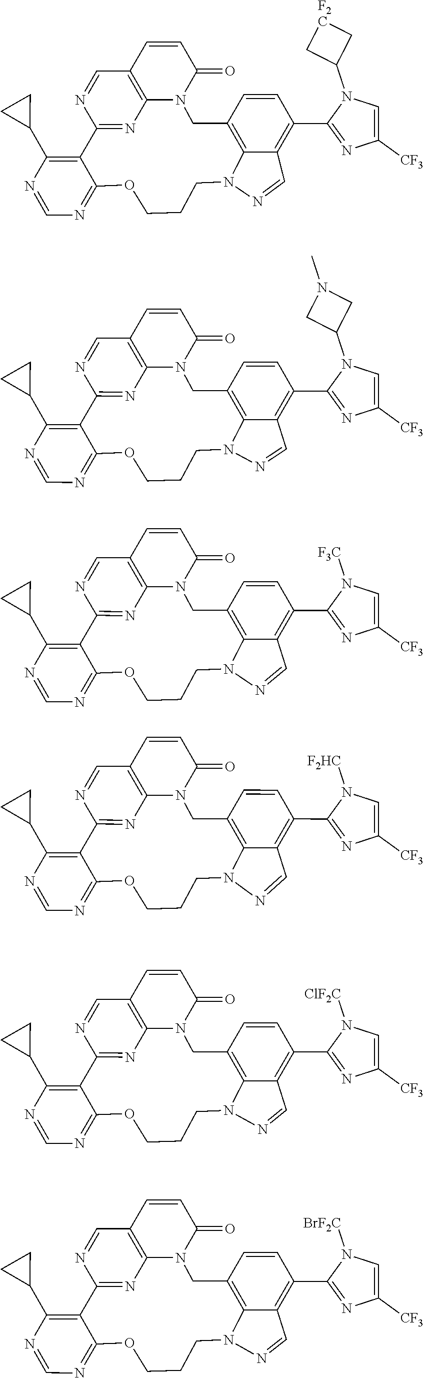

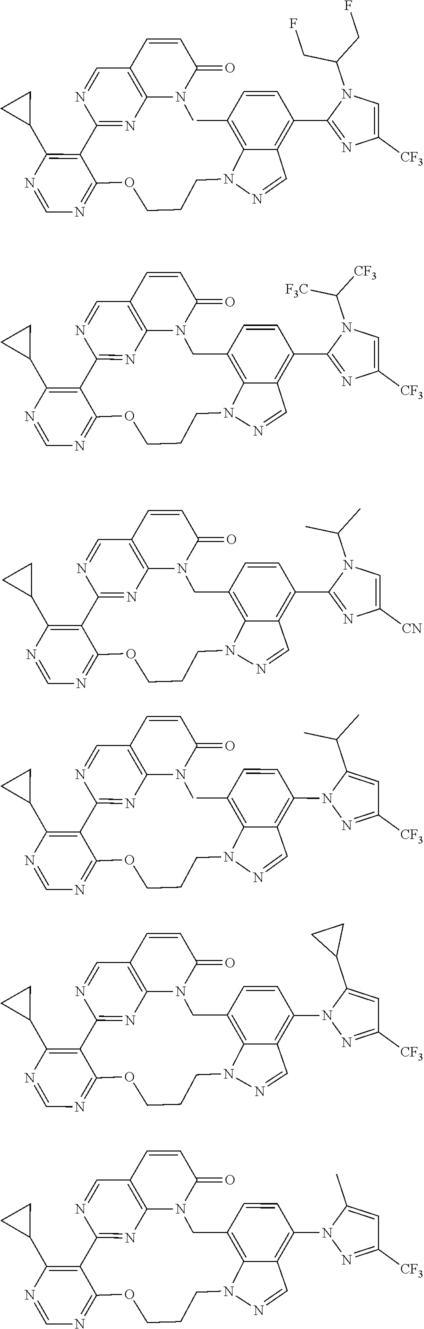

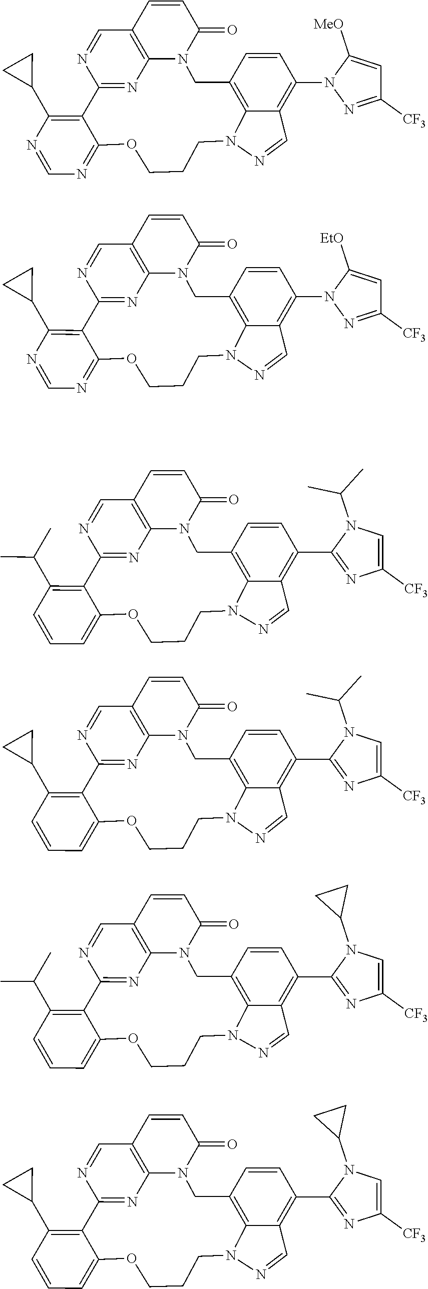

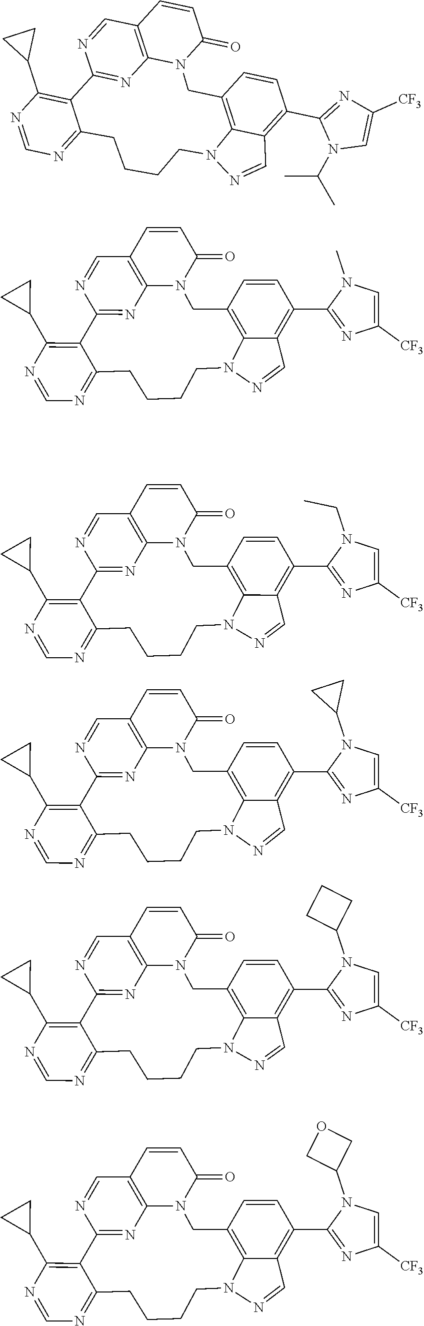

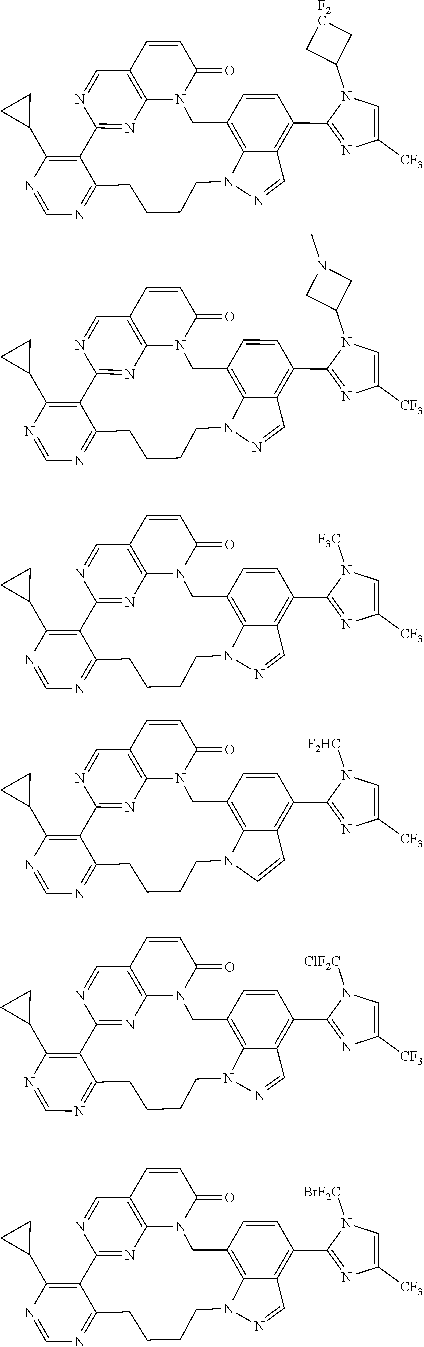

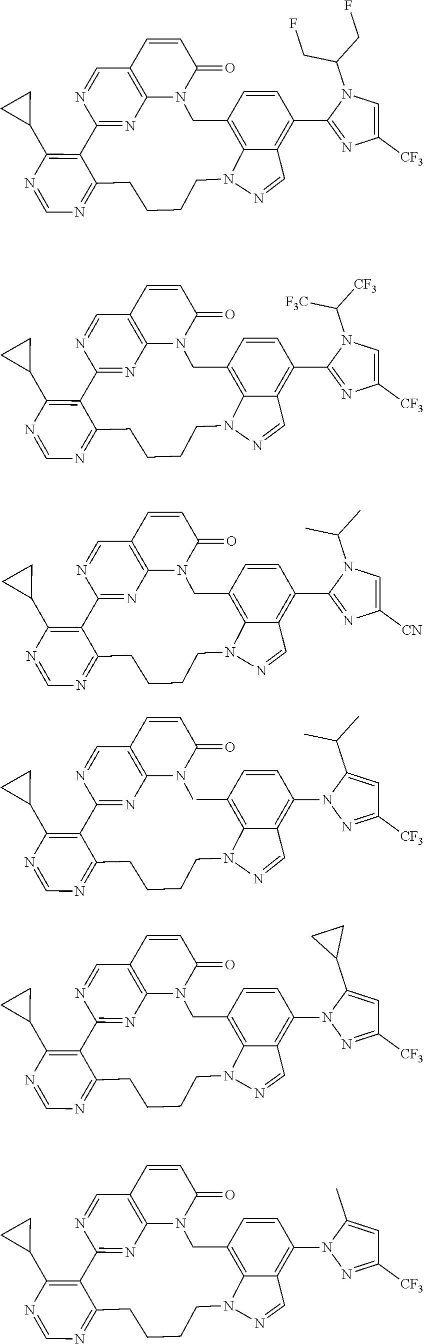

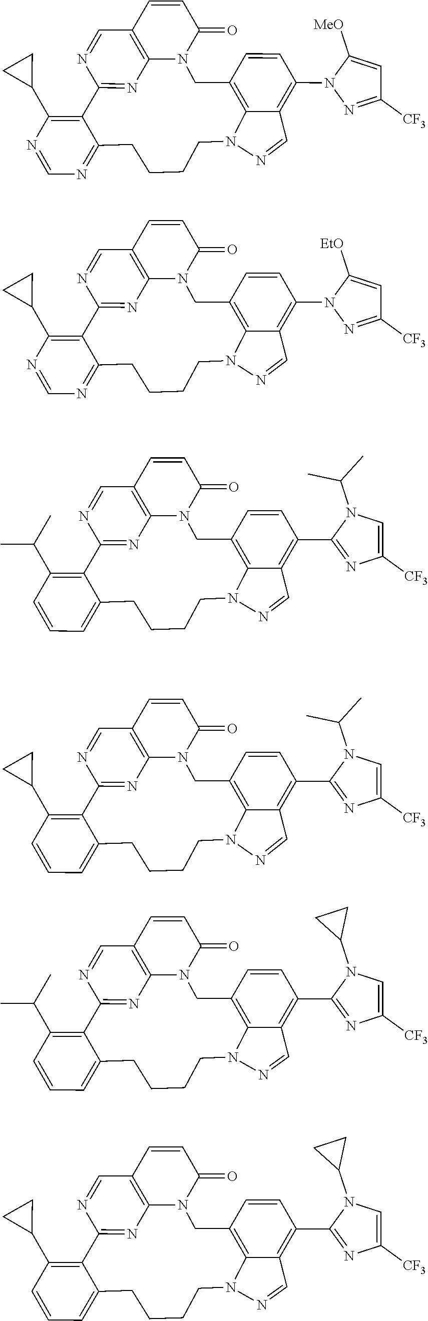

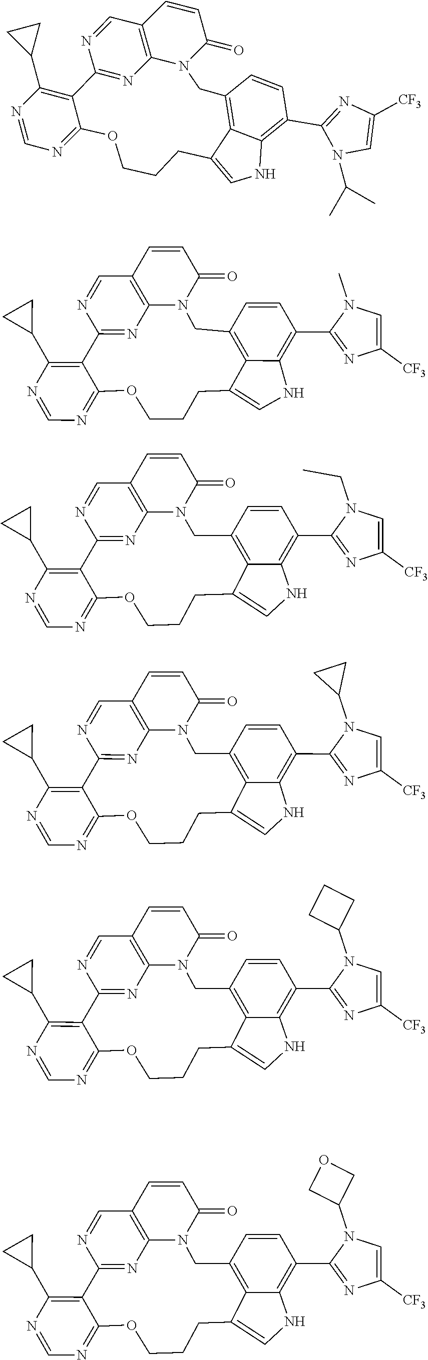

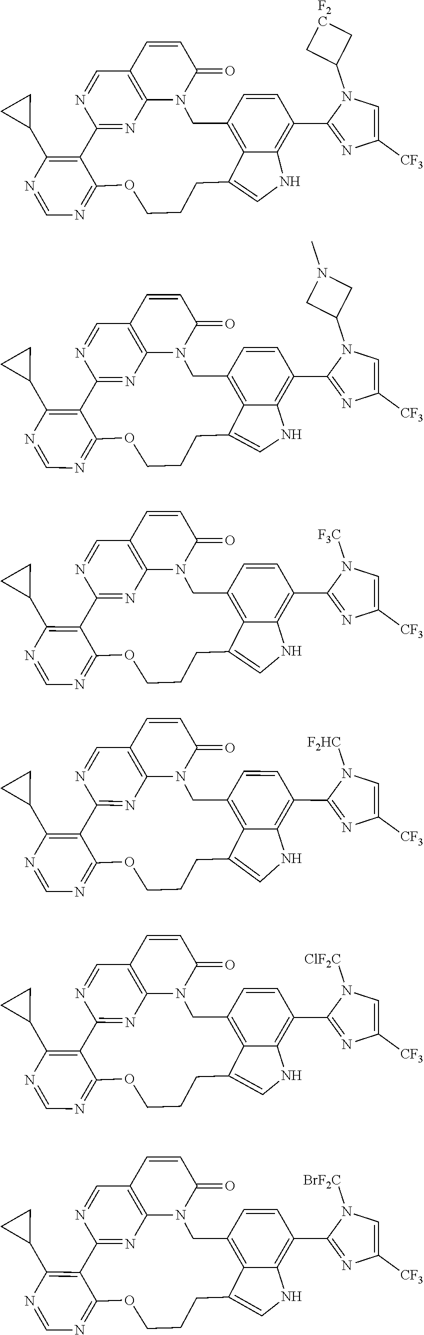

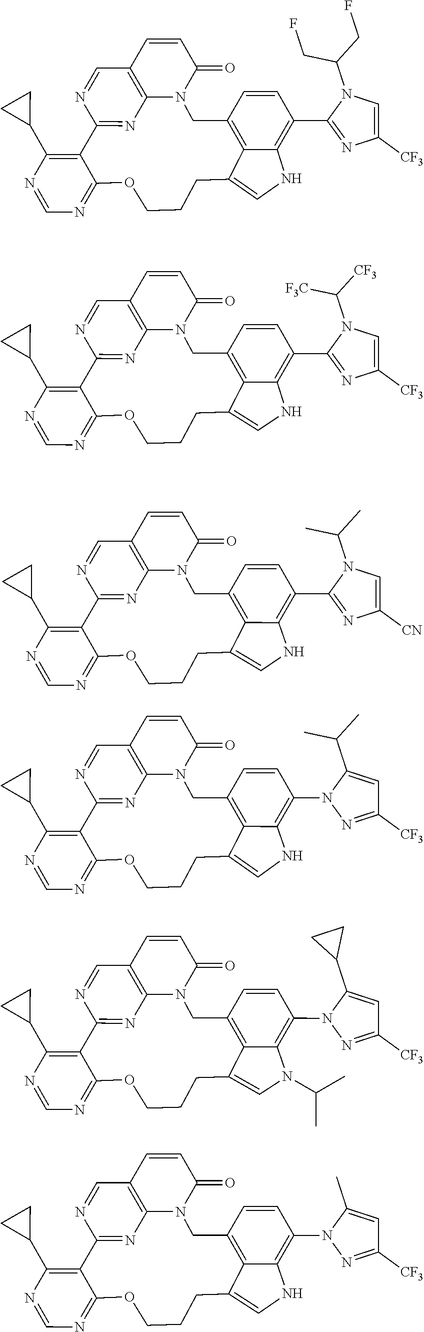

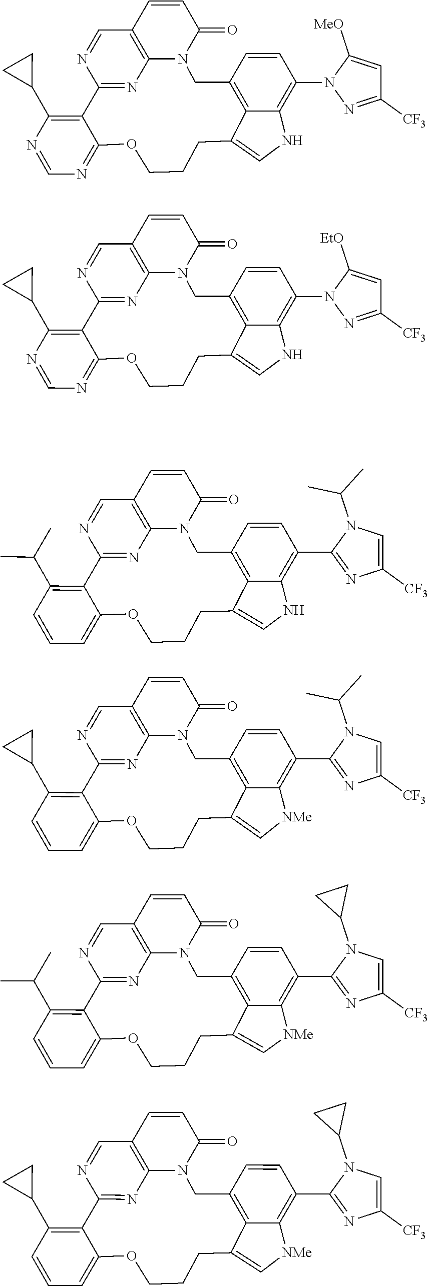

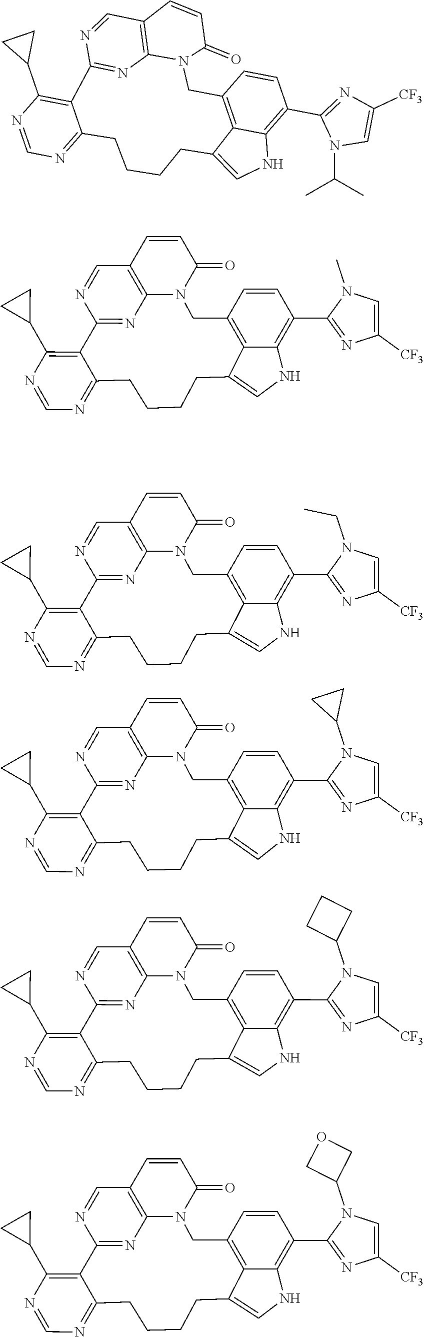

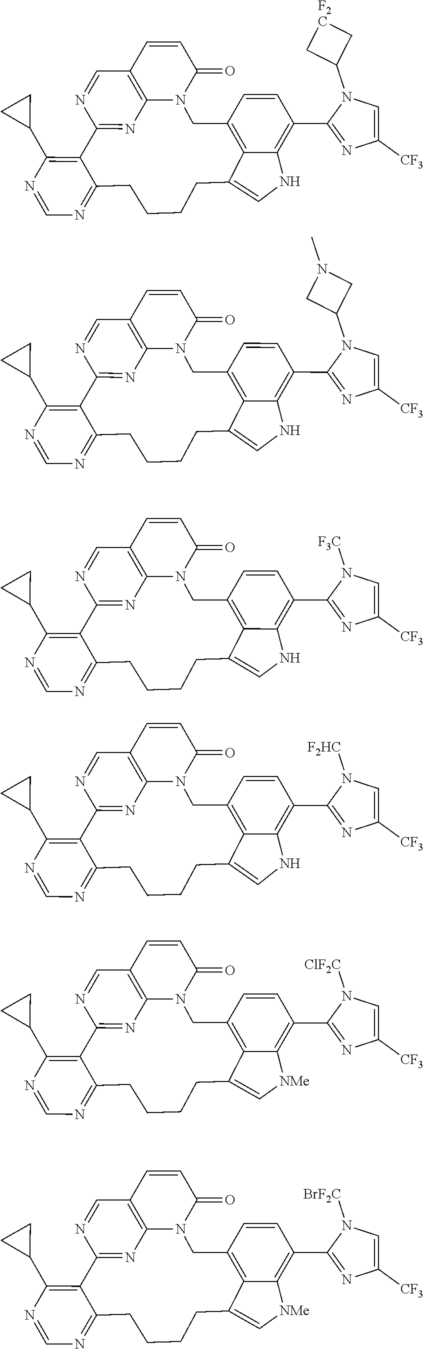

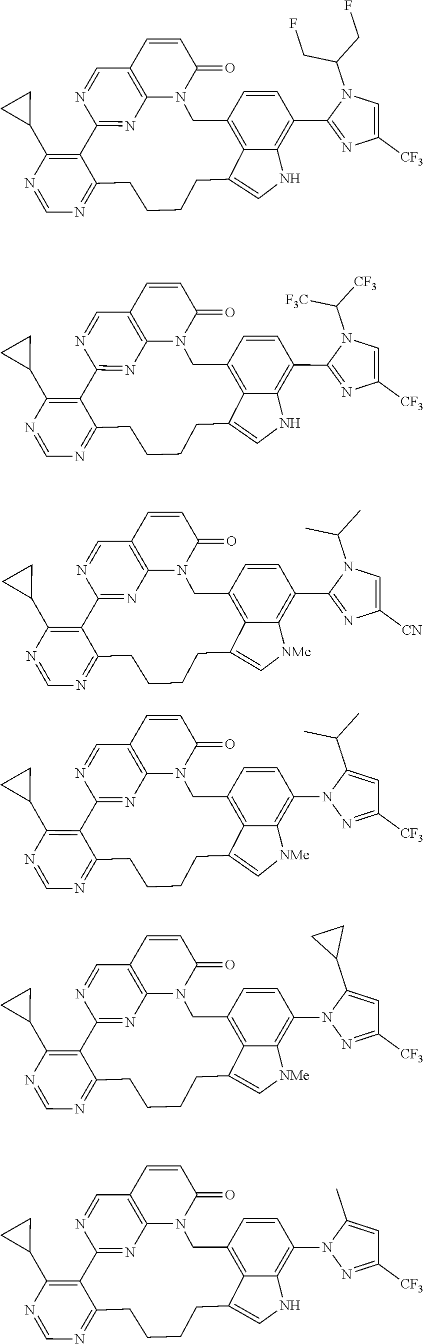

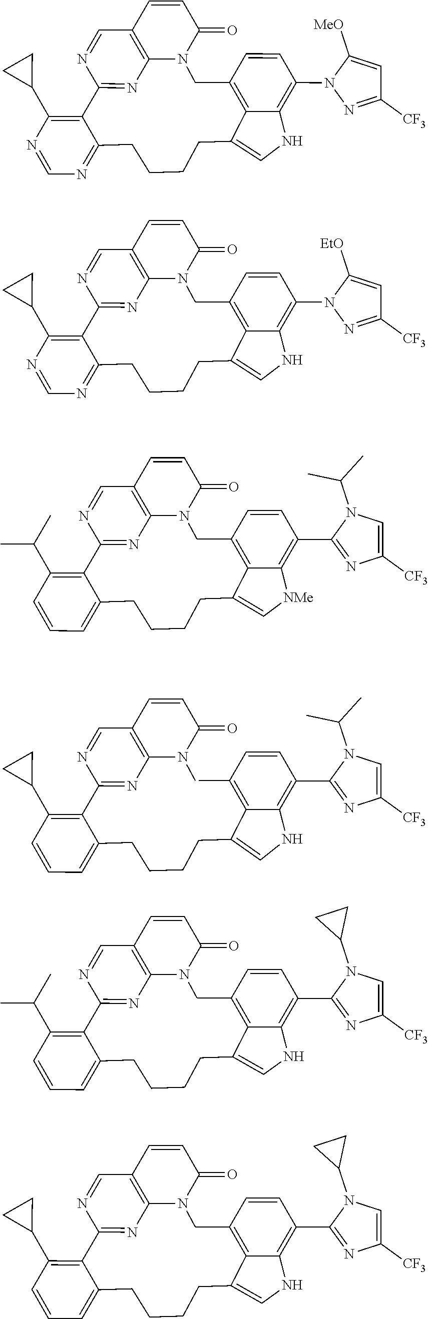

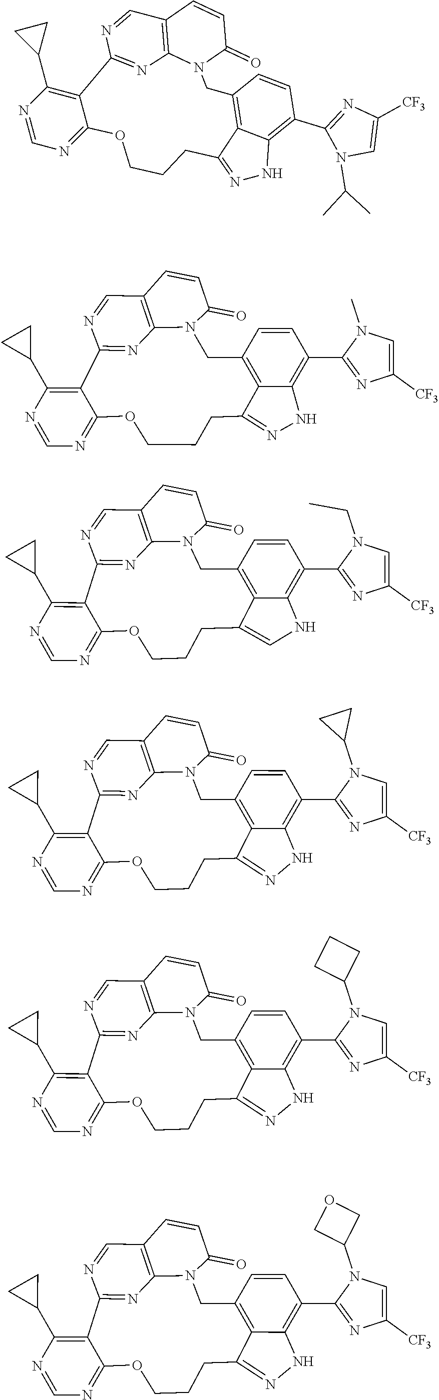

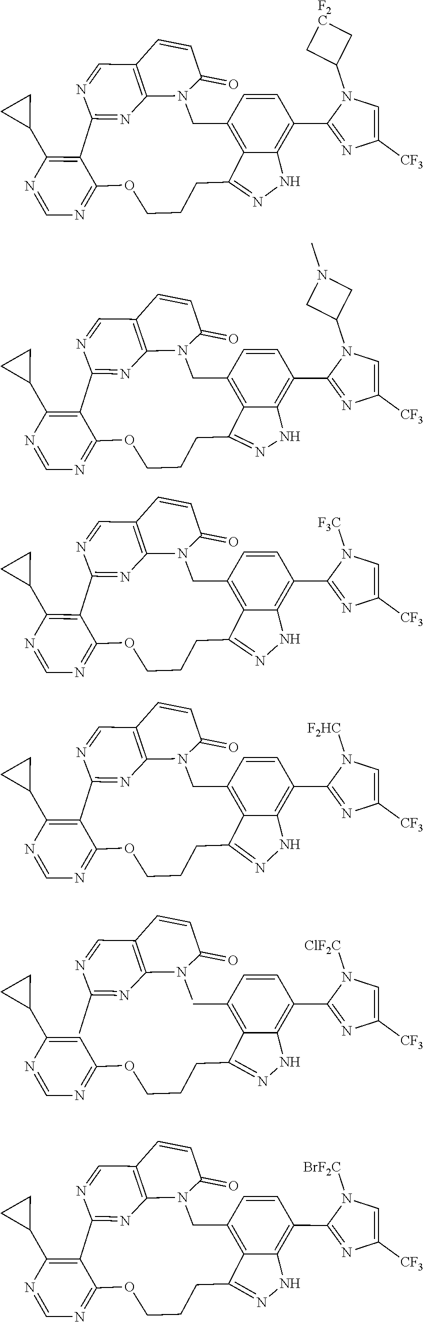

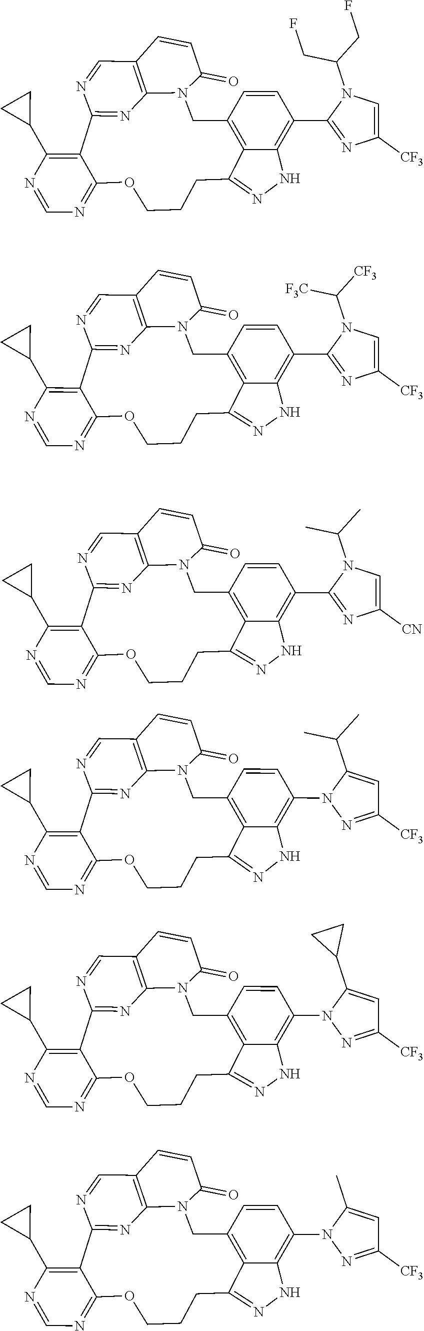

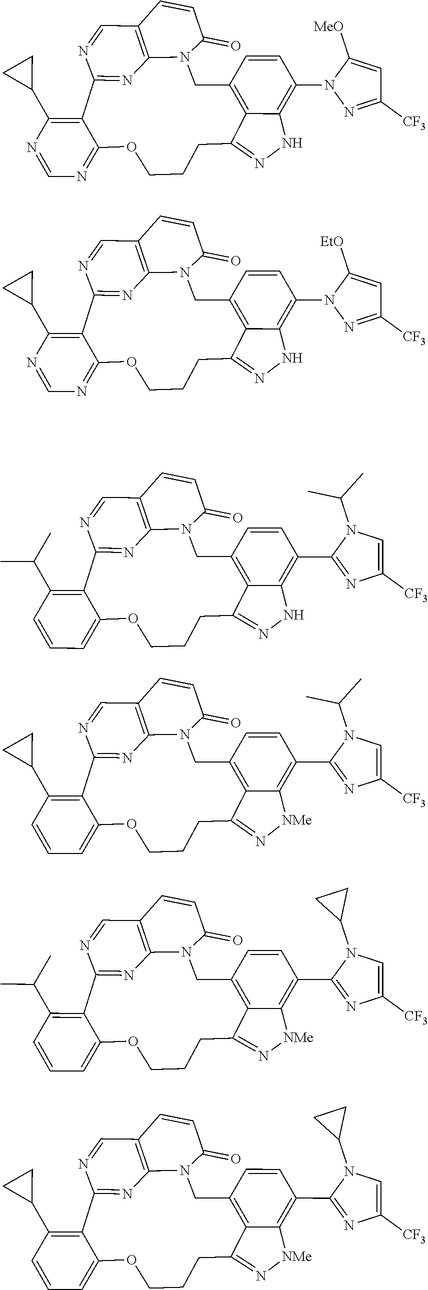

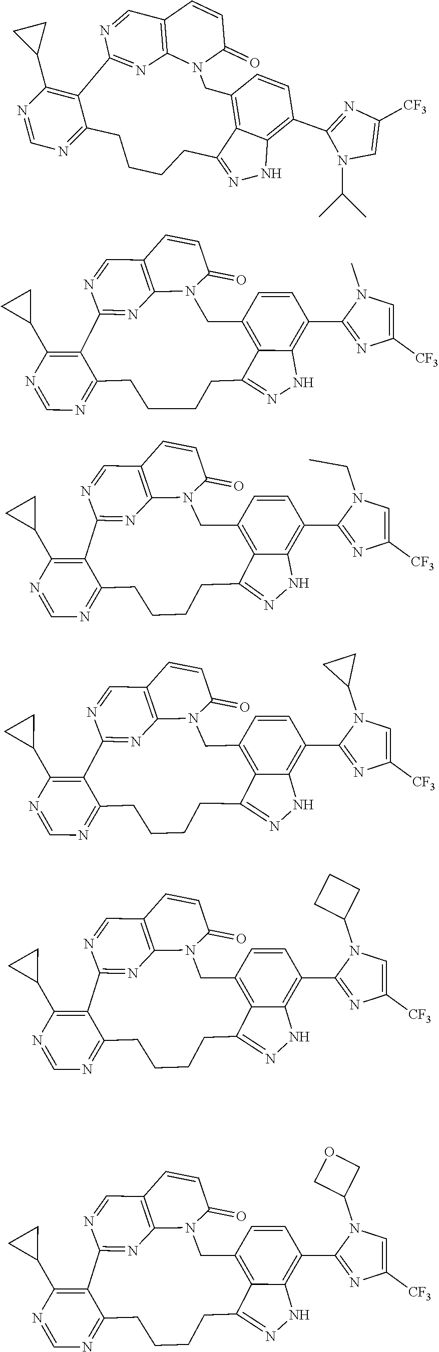

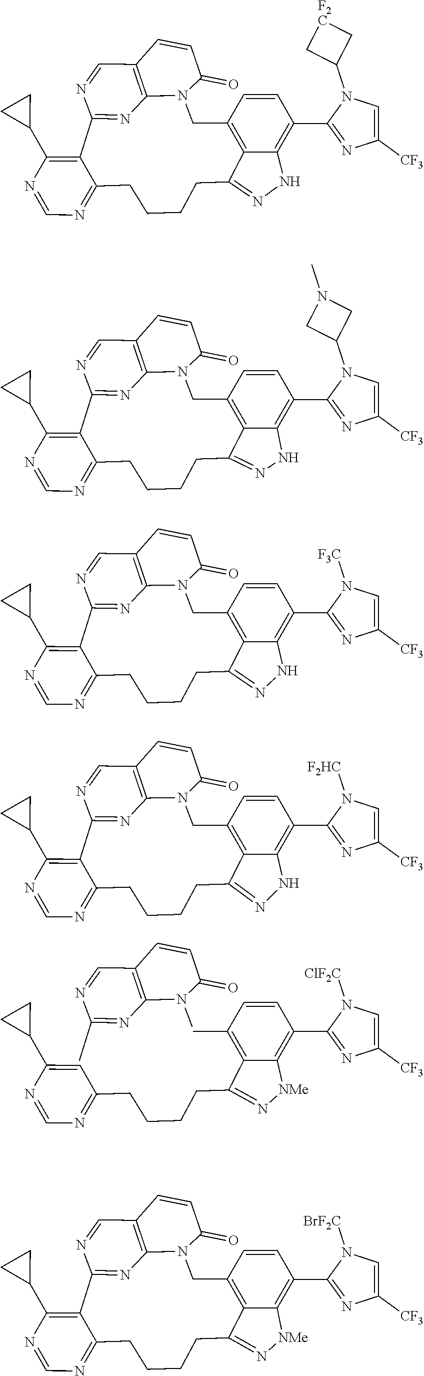

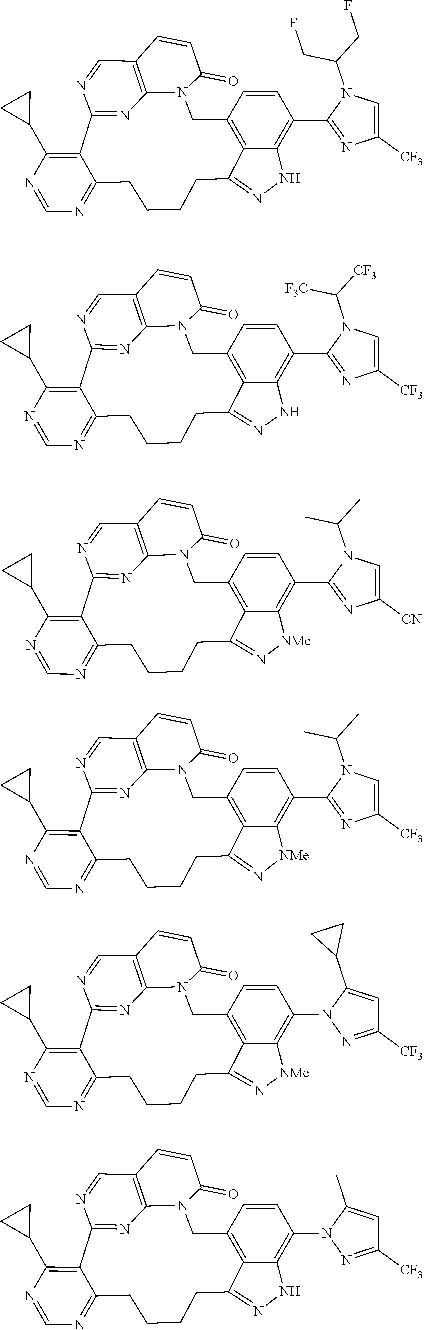

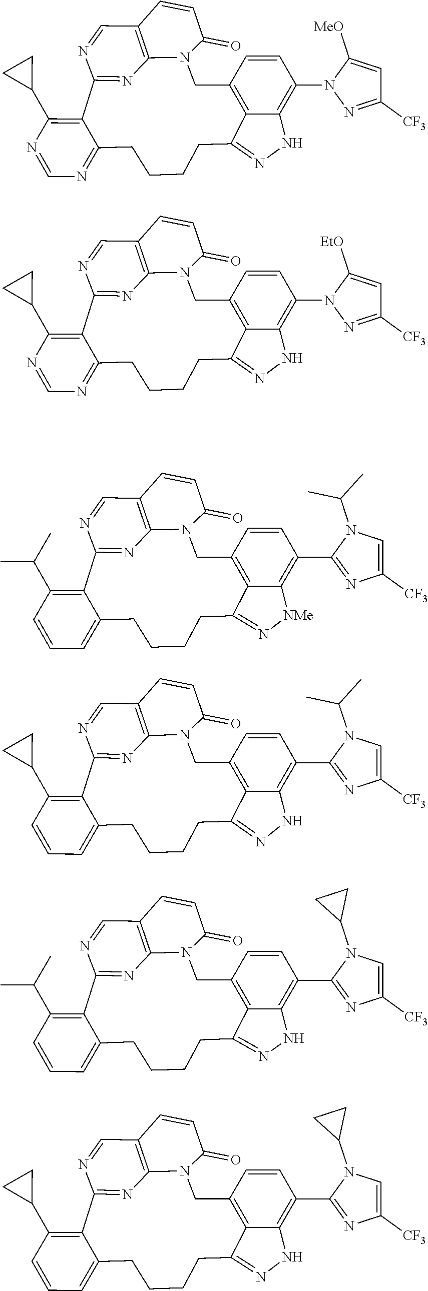

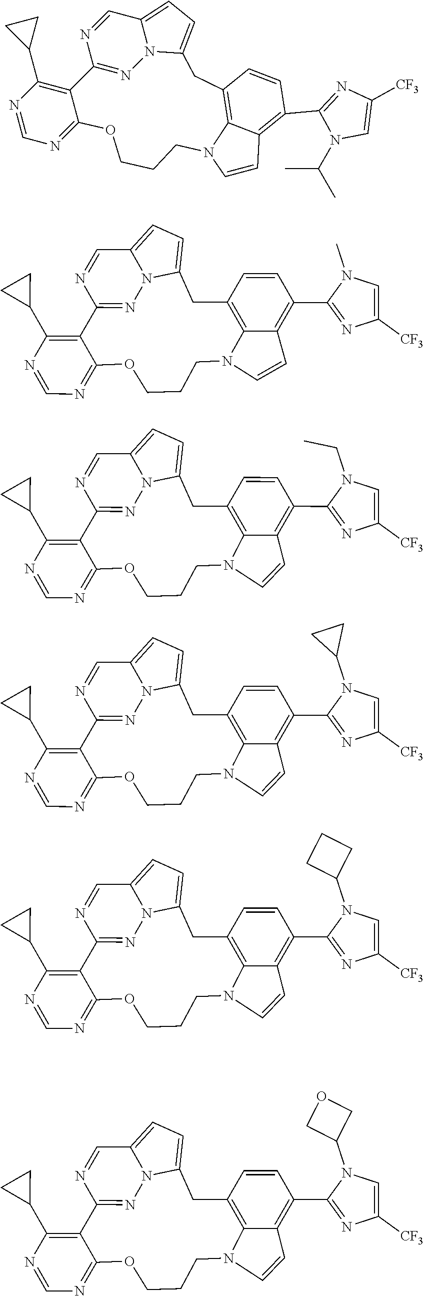

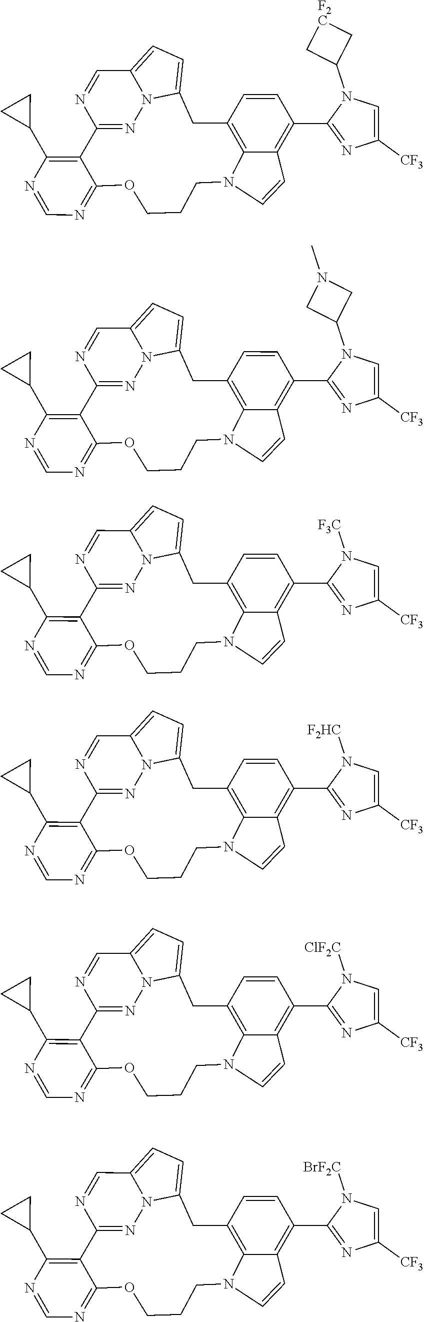

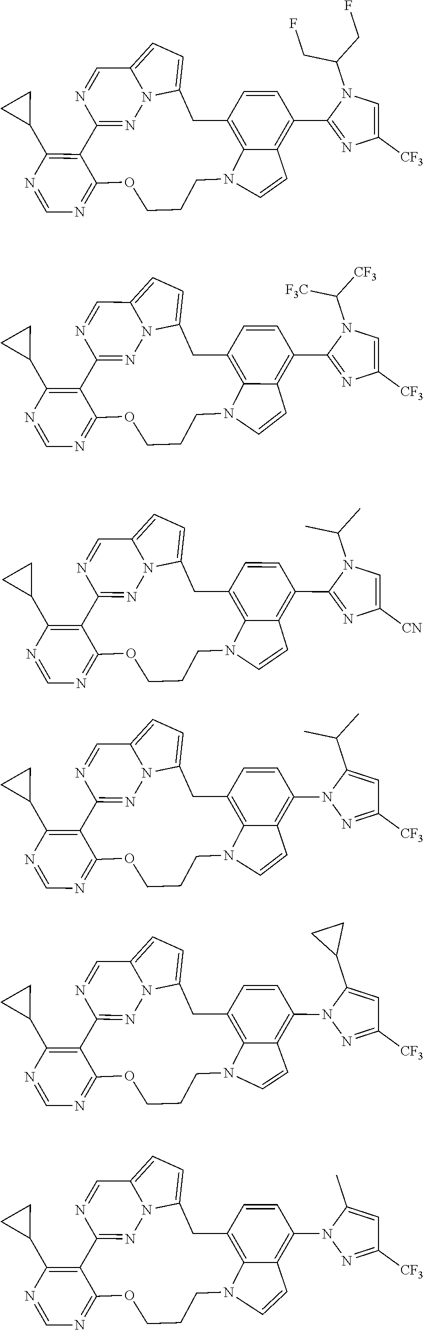

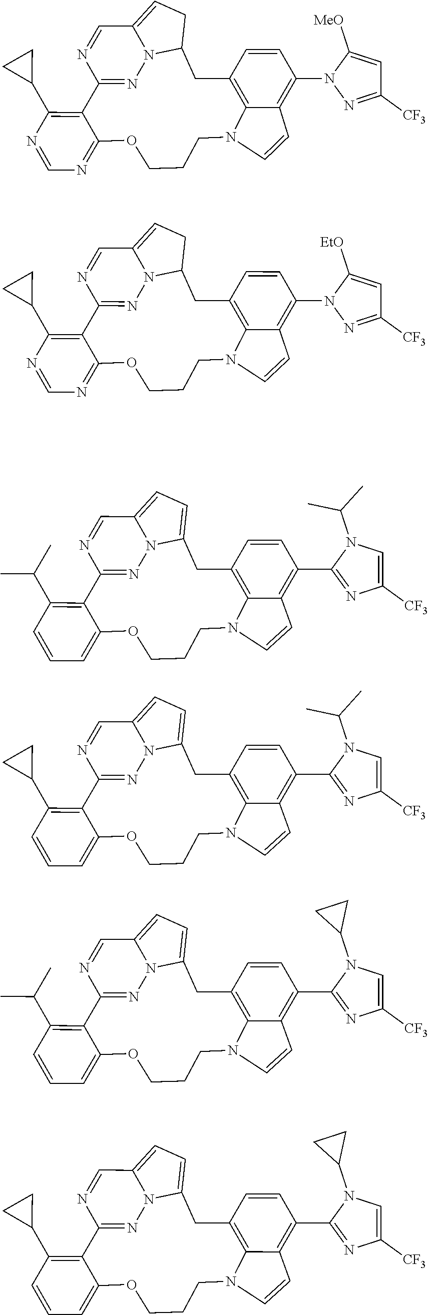

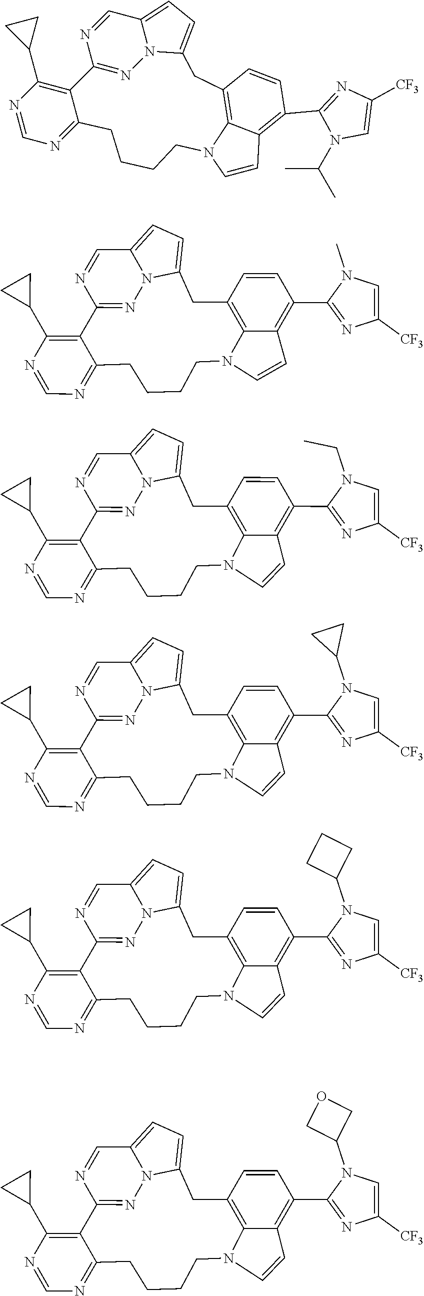

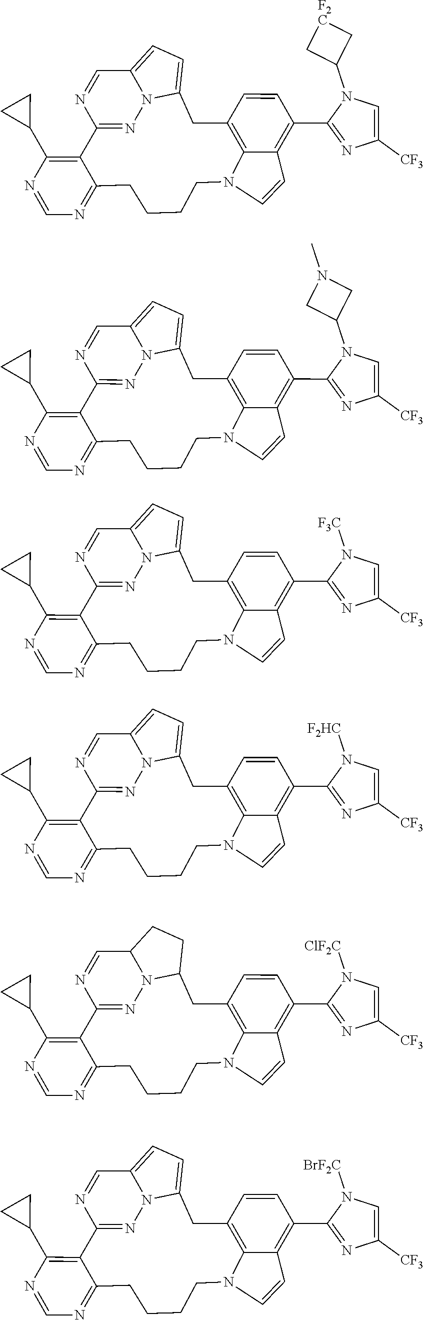

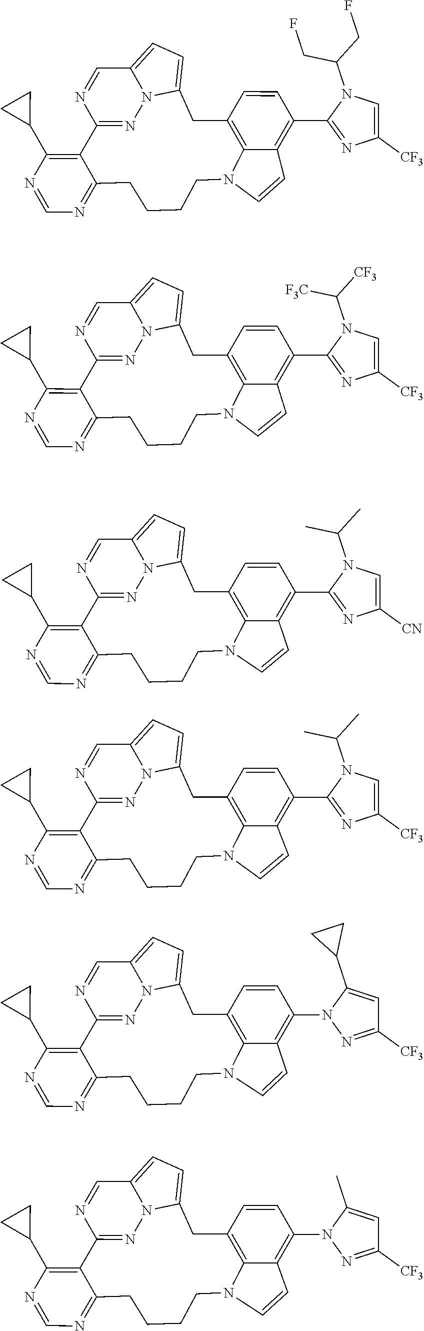

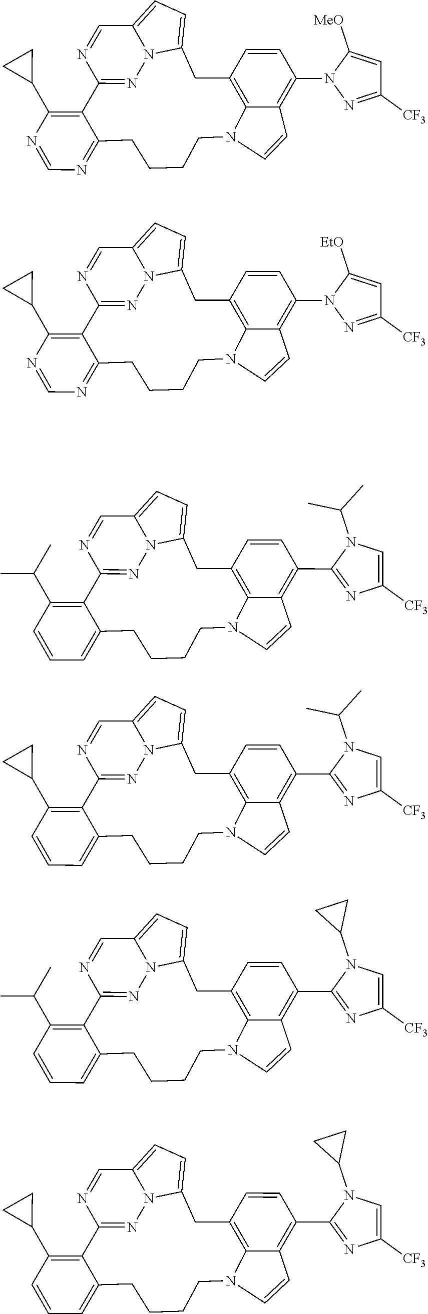

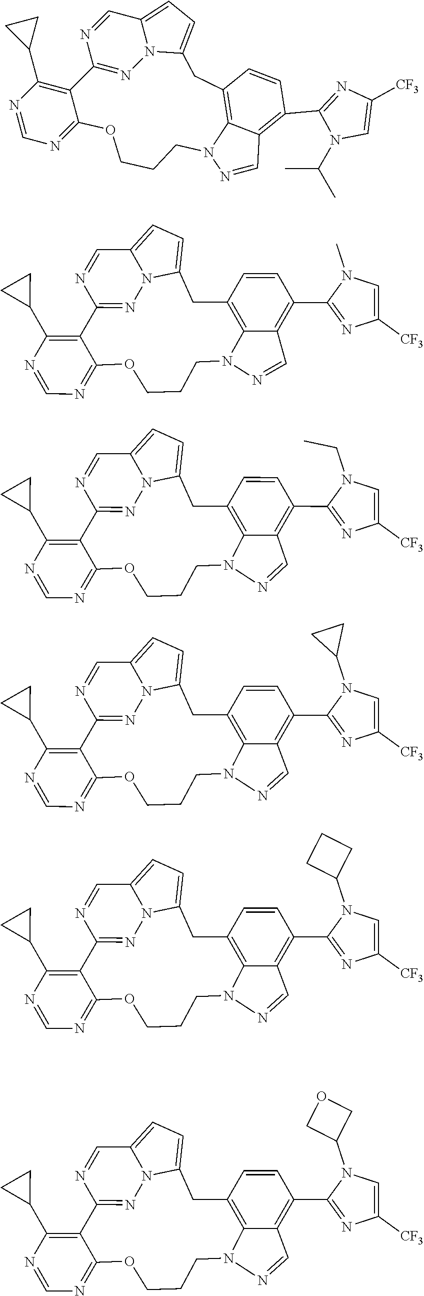

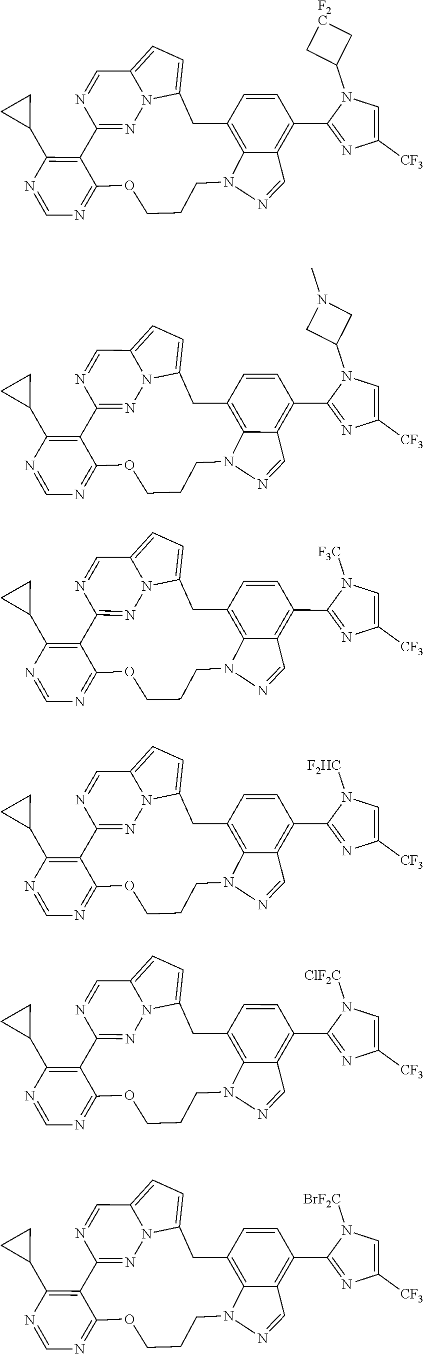

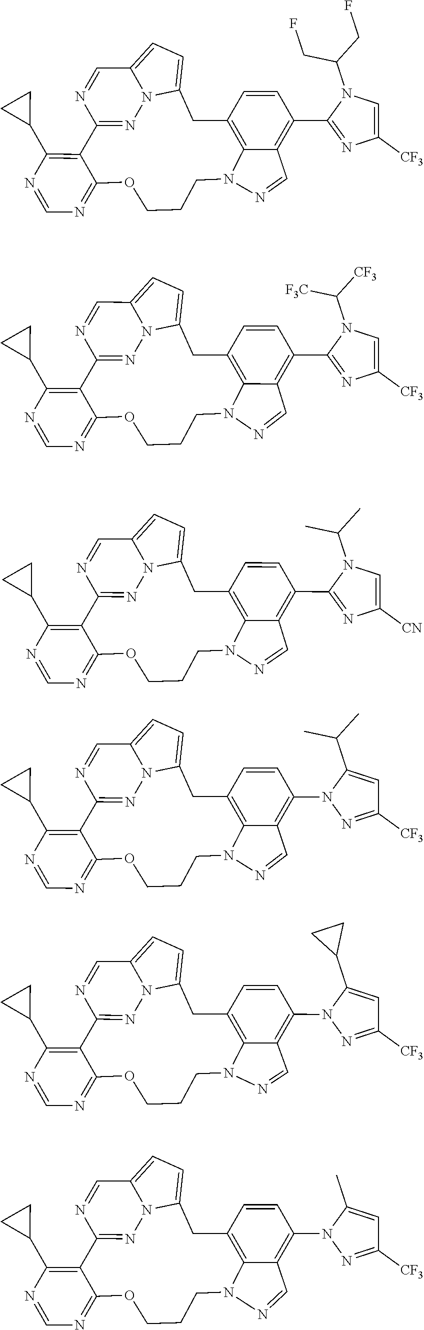

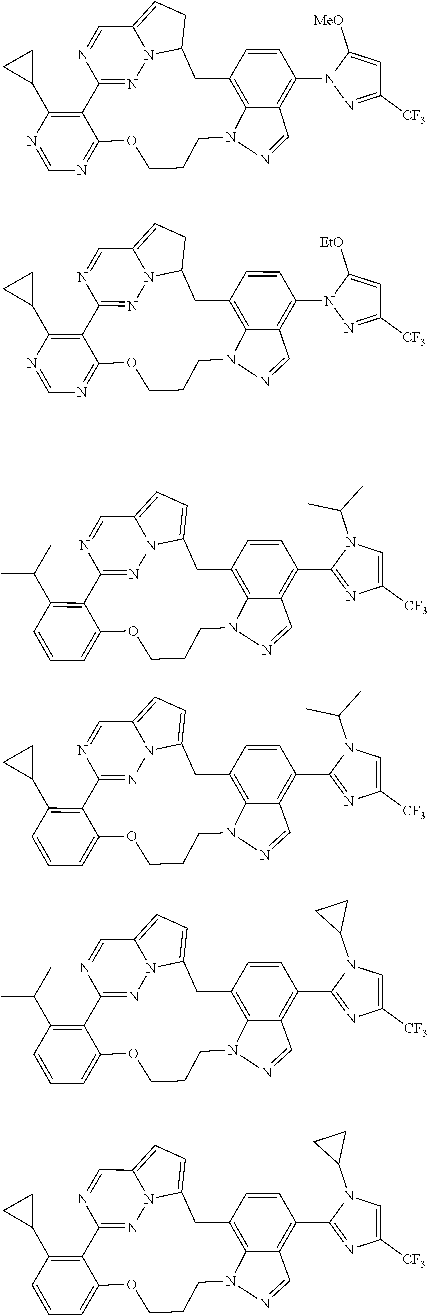

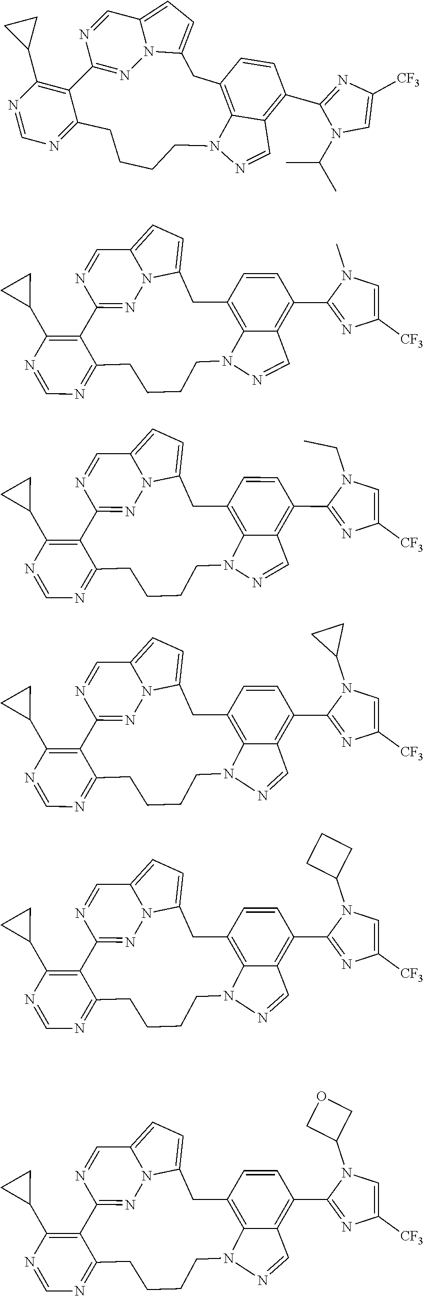

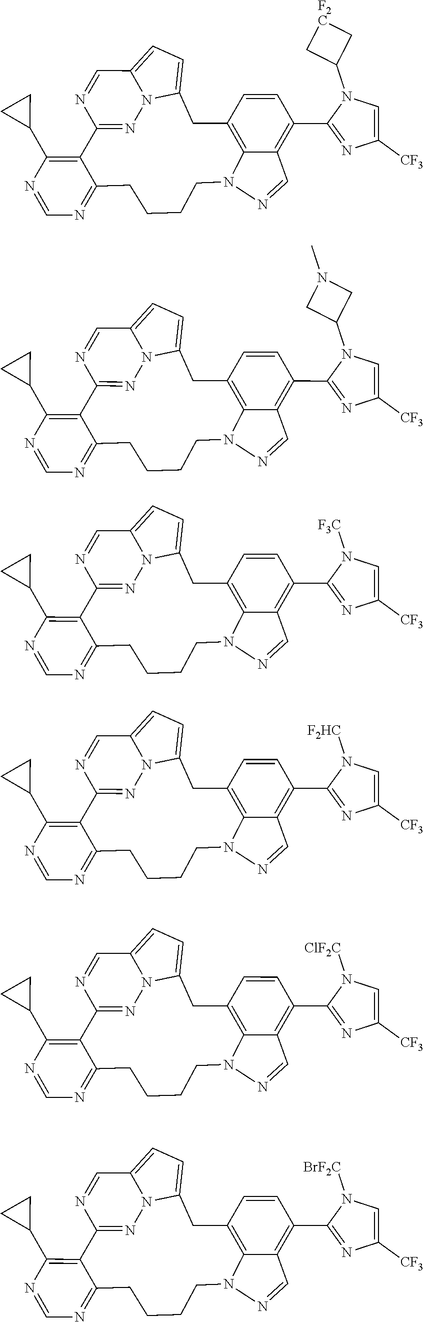

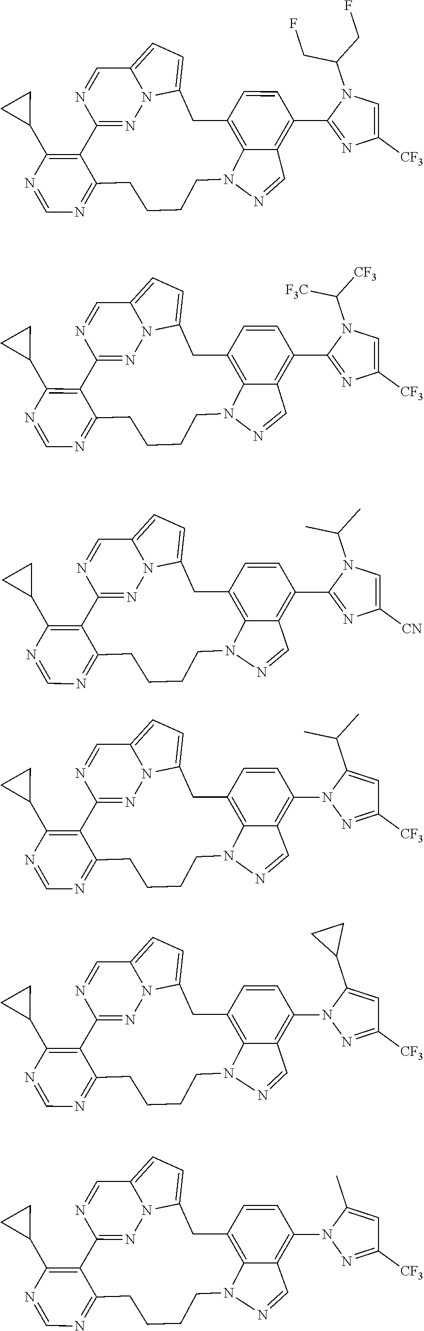

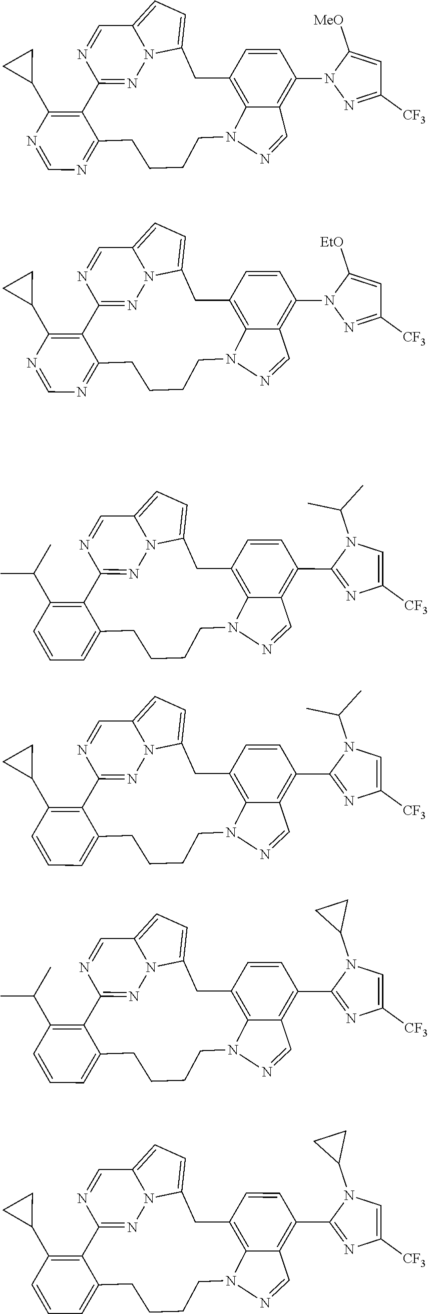

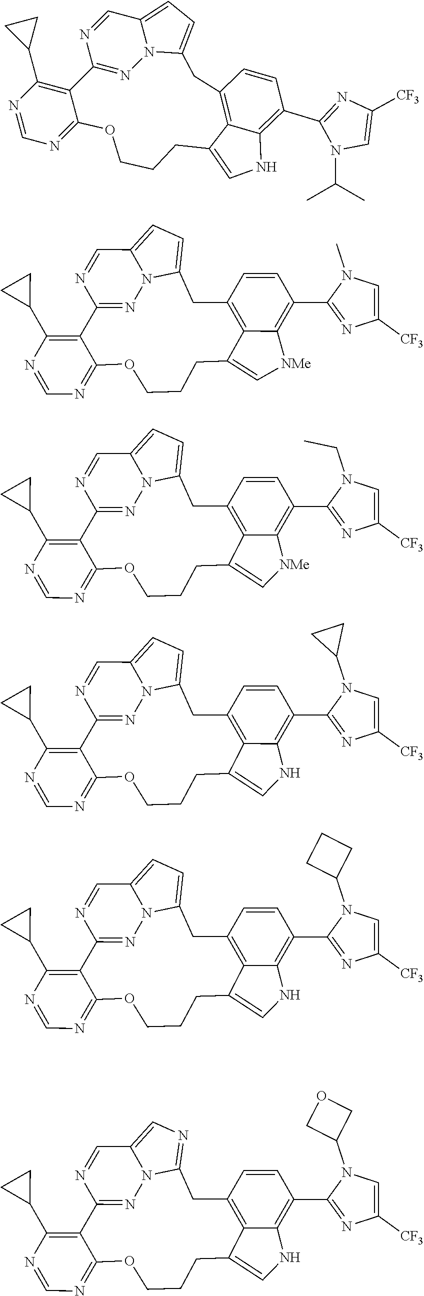

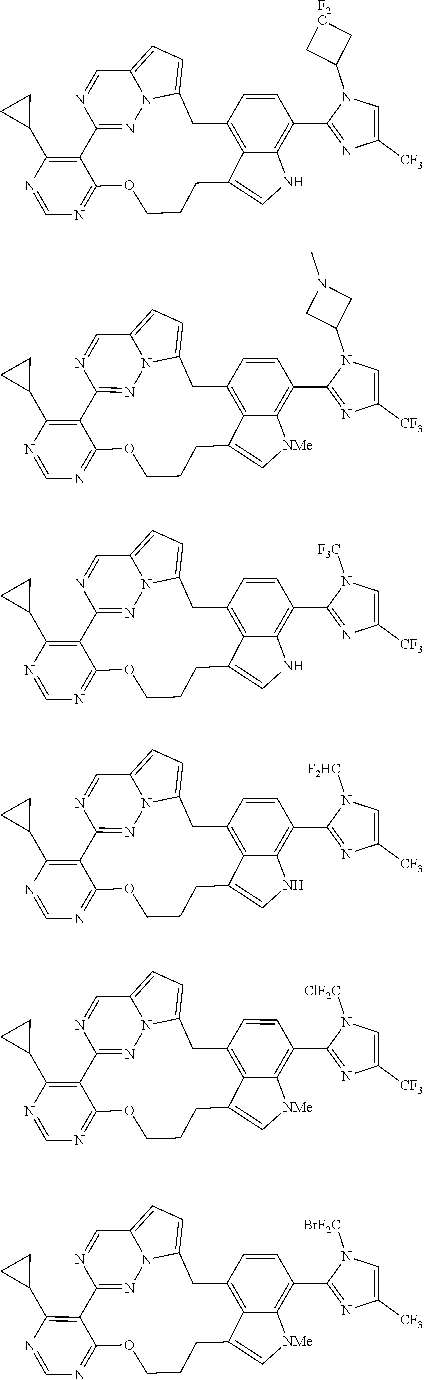

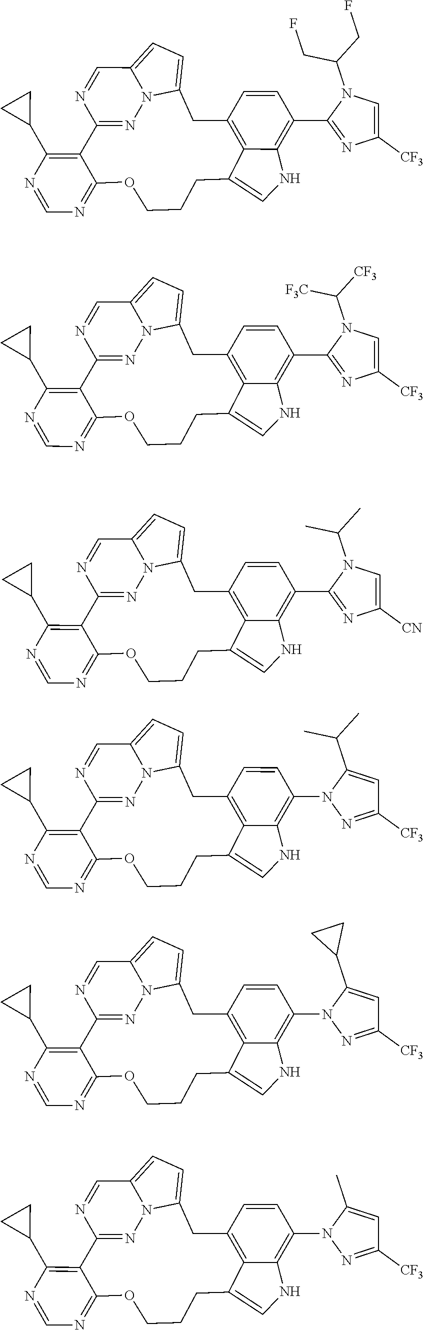

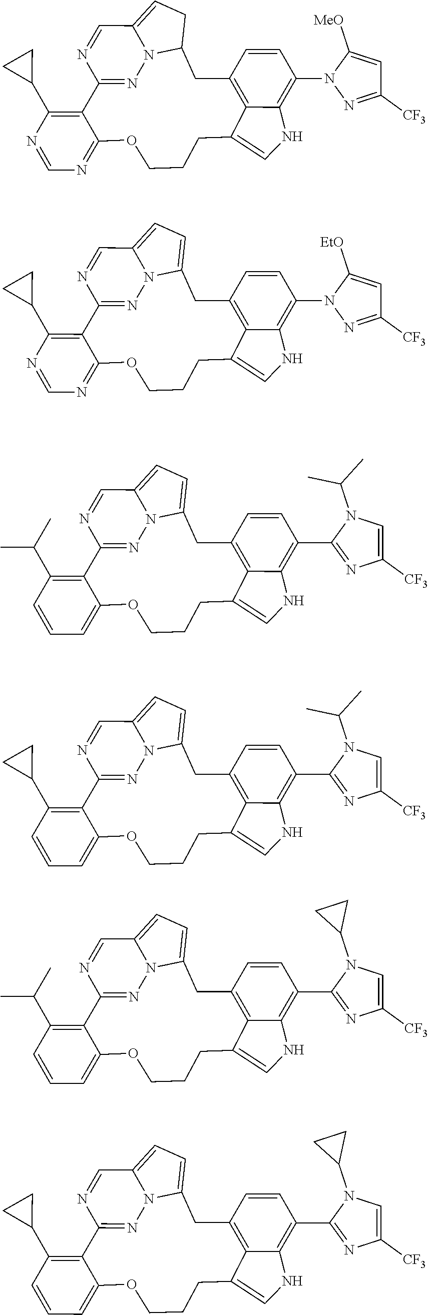 [SMILES: COc1cc(C(F)(F)F)nn1-c1ccc2c3c(c[nH]c13)CCCOc1ncnc(C3CC3)c1C1=NN3C(=CCC3C2)C=N1 ; CCOc1cc(C(F)(F)F)nn1-c1ccc2c3c(c[nH]c13)CCCOc1ncnc(C3CC3)c1-c1ncc3ccc(n3n1)C2 ; CC(C)c1cccc2c1-c1ncc3ccc(n3n1)Cc1ccc(-c3nc(C(F)(F)F)cn3C(C)C)c3[nH]cc(c13)CCCO2 ; CC(C)n1cc(C(F)(F)F)nc1-c1ccc2c3c(c[nH]c13)CCCOc1cccc(C3CC3)c1-c1ncc3ccc(n3n1)C2 ; CC(C)c1cccc2c1-c1ncc3ccc(n3n1)Cc1ccc(-c3nc(C(F)(F)F)cn3C3CC3)c3[nH]cc(c13)CCCO2 ; FC(F)(F)c1cn(C2CC2)c(-c2ccc3c4c(c[nH]c24)CCCOc2cccc(C4CC4)c2-c2ncc4ccc(n4n2)C3)n1]

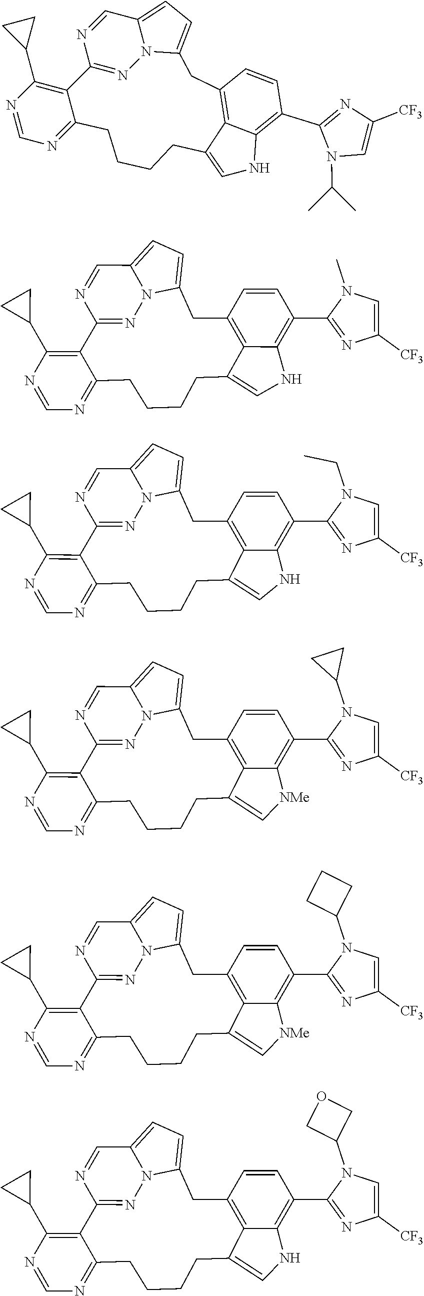

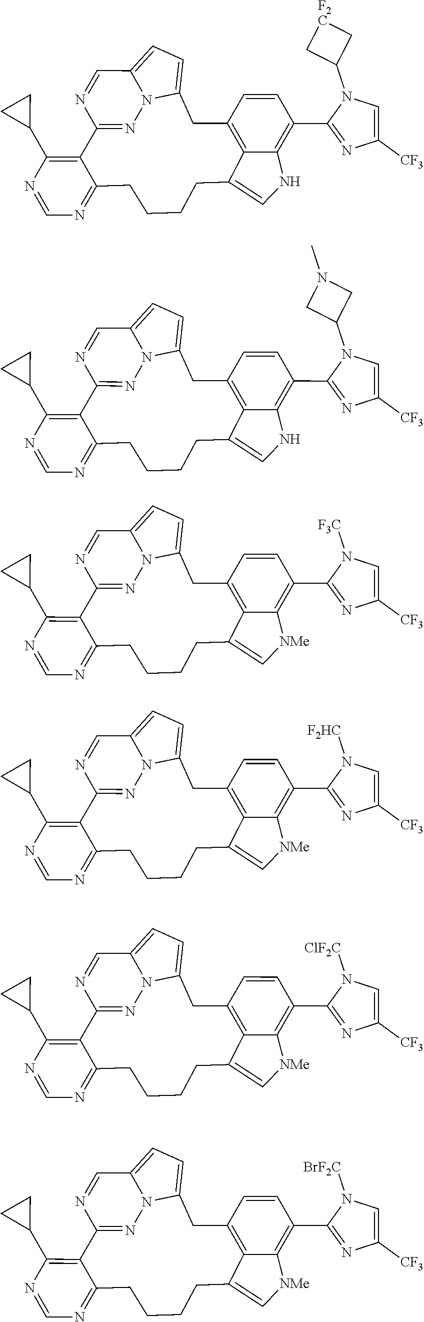

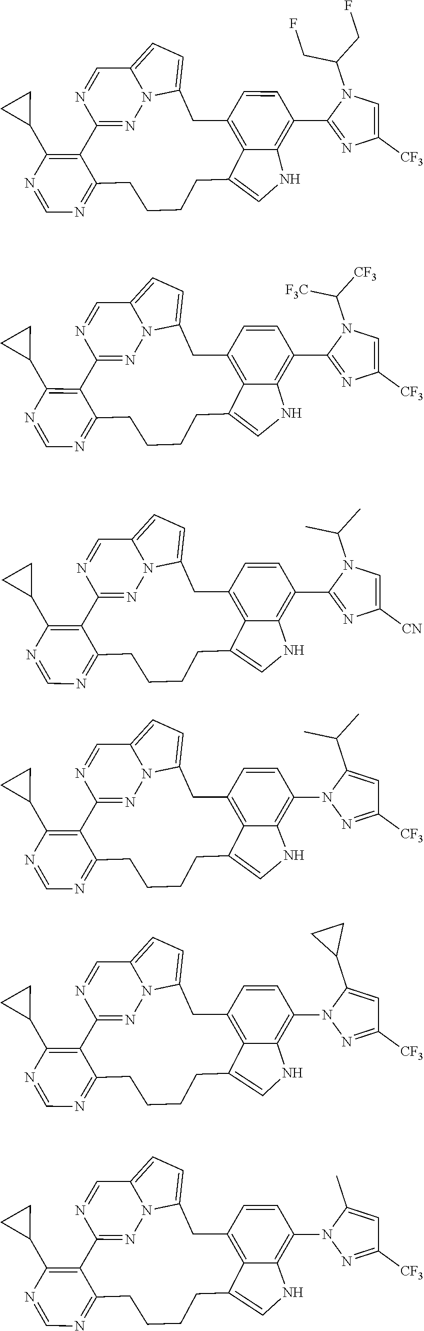

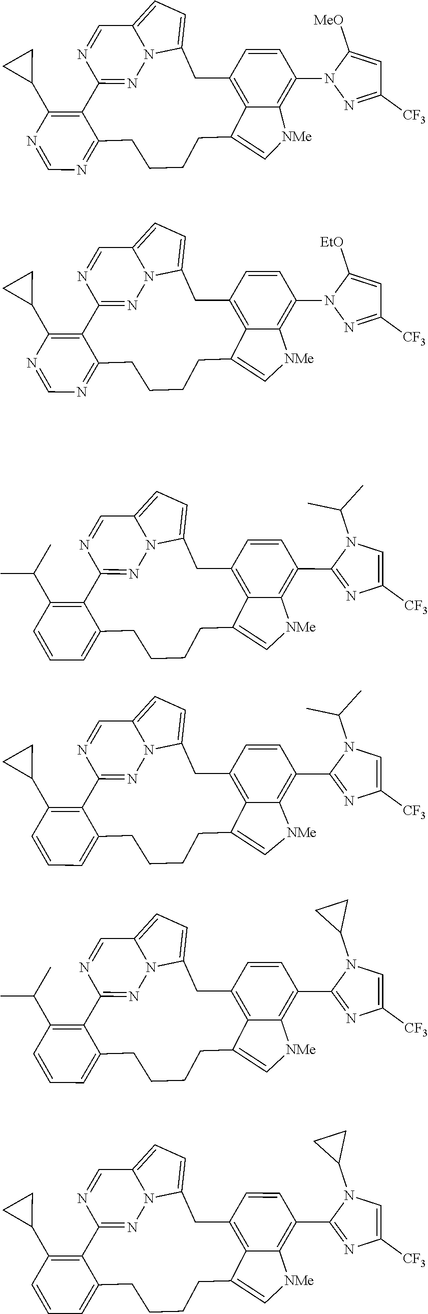

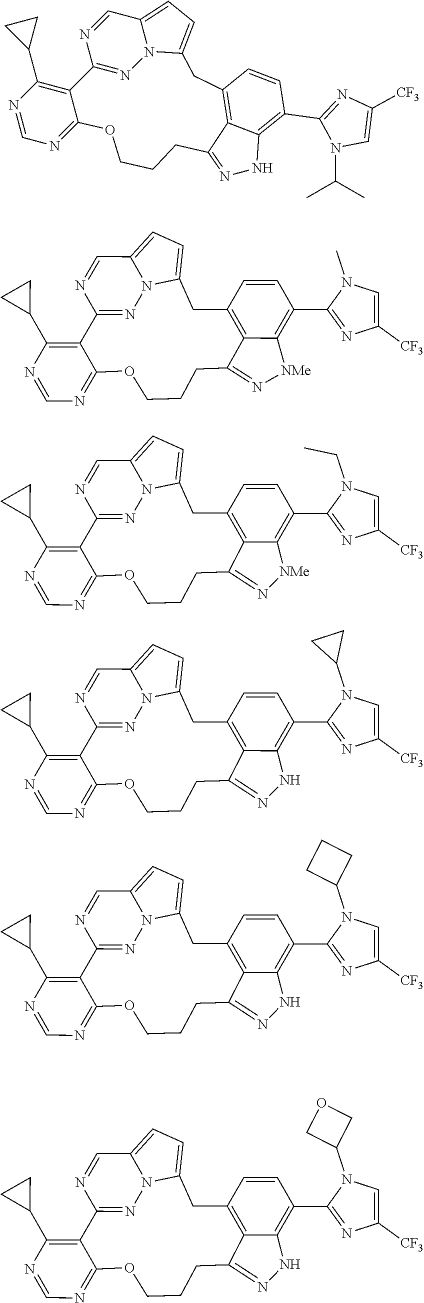

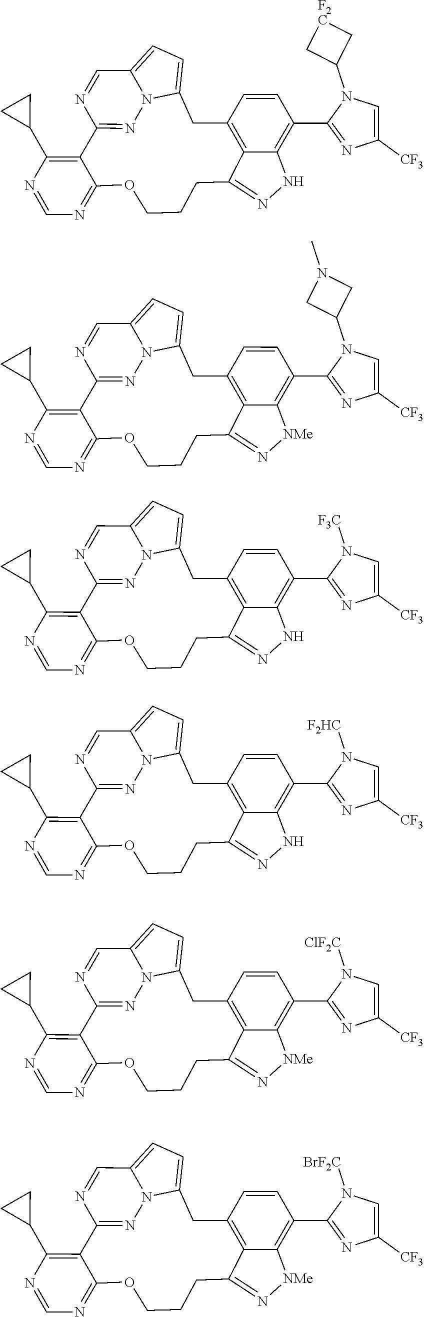

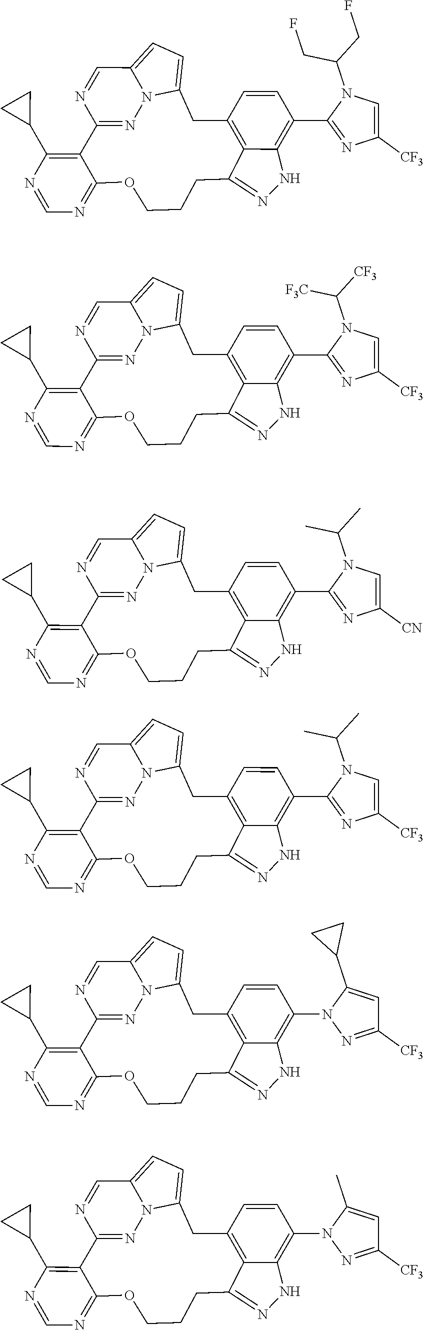

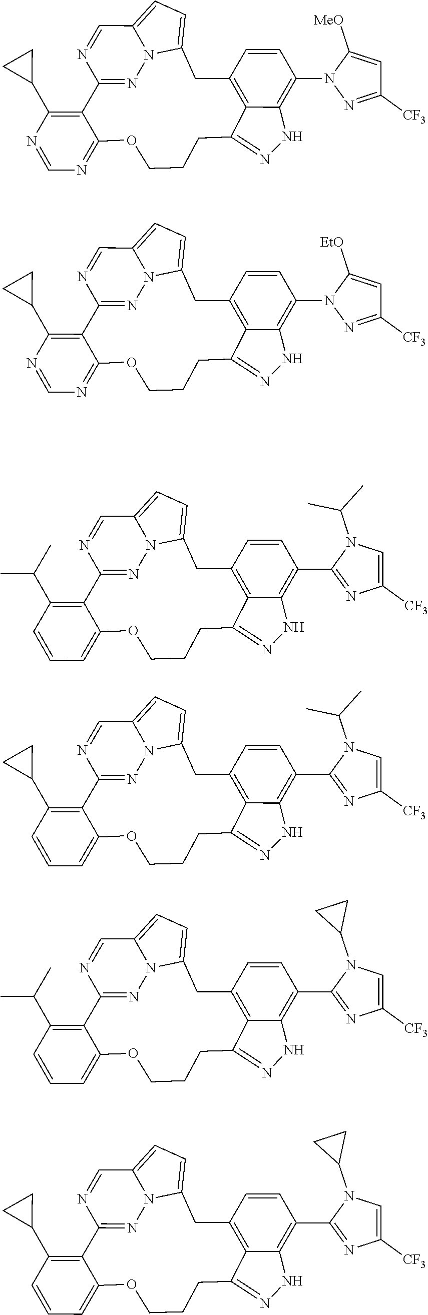

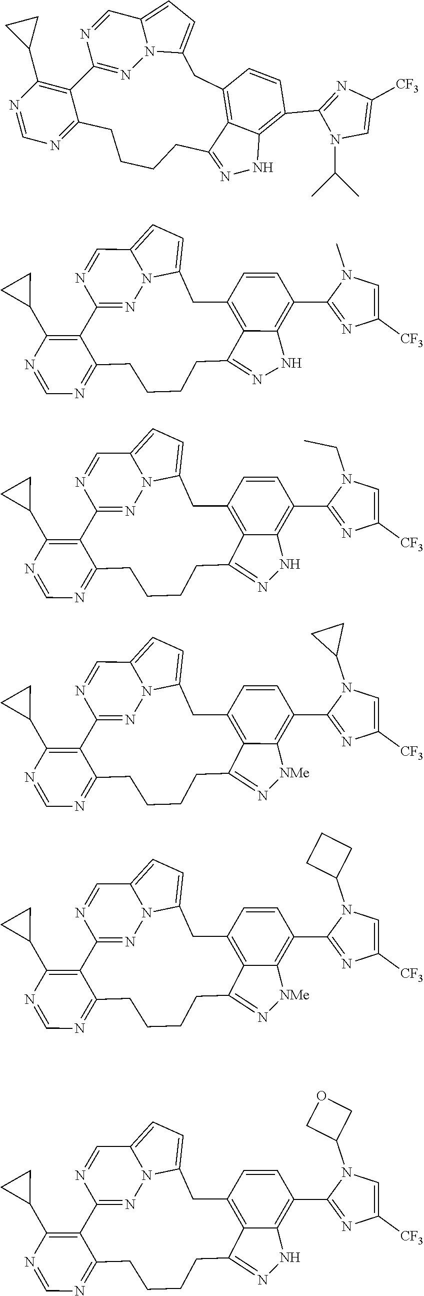

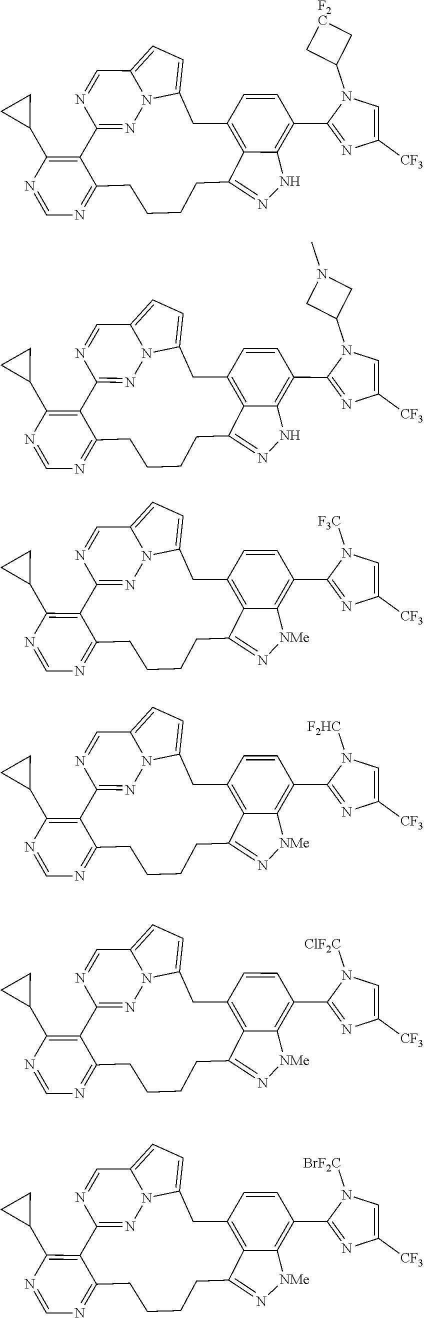

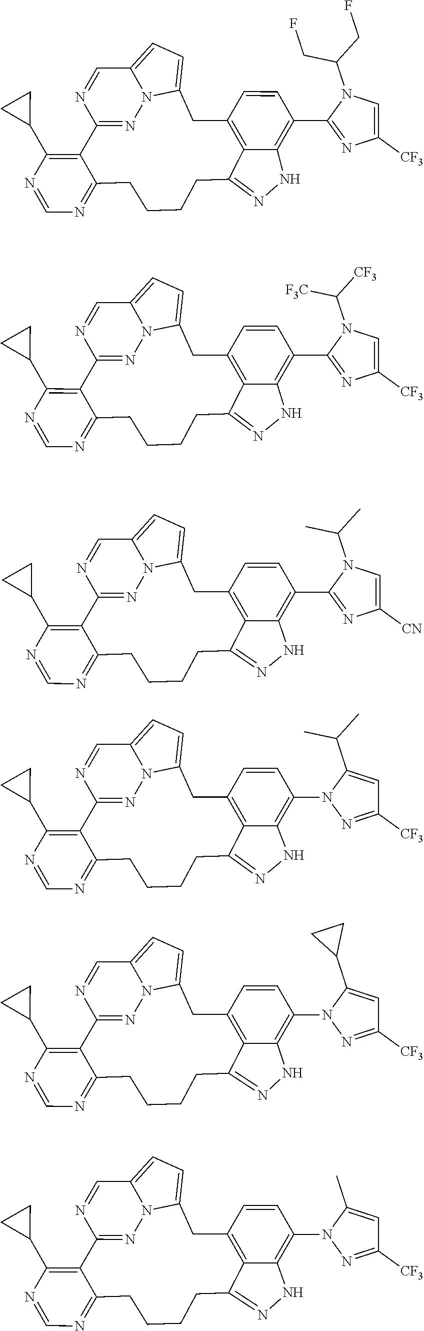

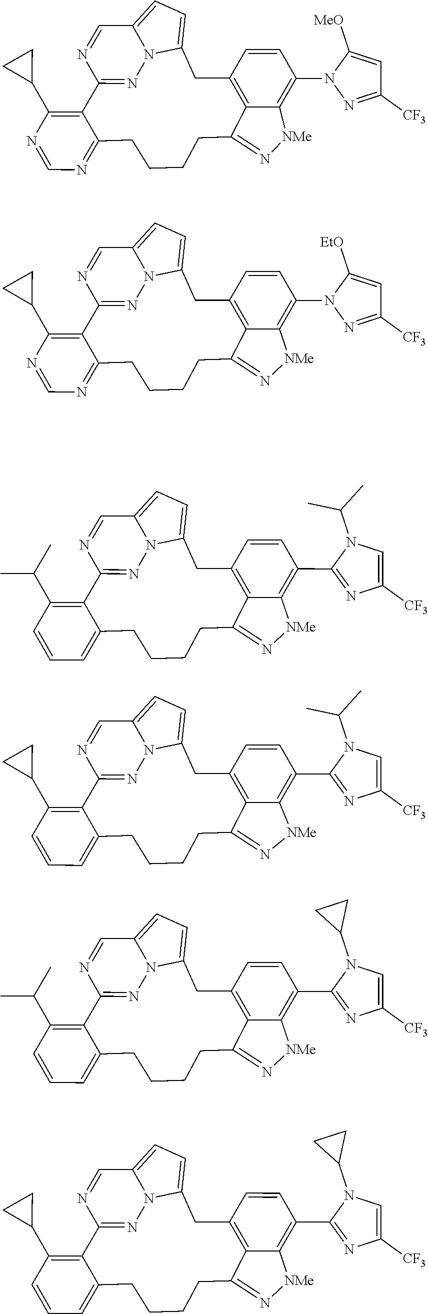

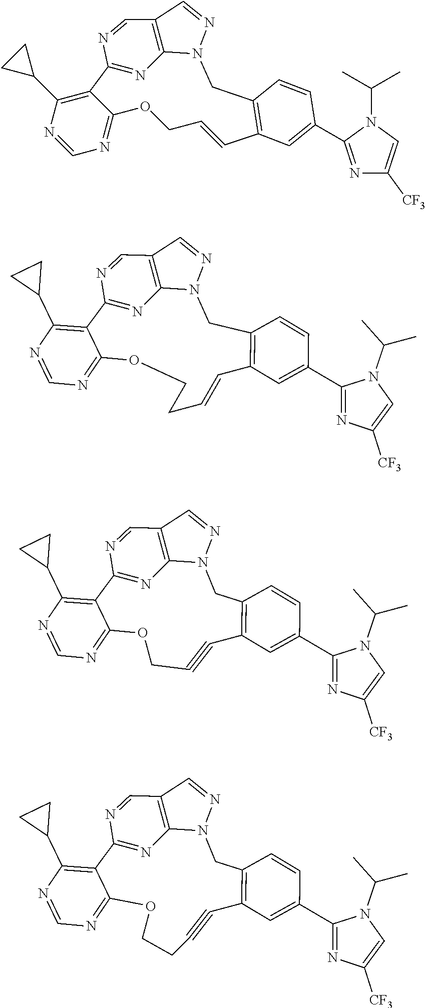 [SMILES: CC(C)n1cc(C(F)(F)F)nc1-c1ccc2c(c1)/C=C/COc1ncnc(C3CC3)c1-c1ncc3cnn(c3n1)C2 ; CC(C)n1cc(C(F)(F)F)nc1-c1ccc2c(c1)/C=C/CCOc1ncnc(C3CC3)c1-c1ncc3cnn(c3n1)C2 ; CC(C)n1cc(C(F)(F)F)nc1-c1ccc2c(c1)C#CCOc1ncnc(C3CC3)c1-c1ncc3cnn(c3n1)C2 ; CC(C)n1cc(C(F)(F)F)nc1-c1ccc2c(c1)C#CCCOc1ncnc(C3CC3)c1-c1ncc3cnn(c3n1)C2]

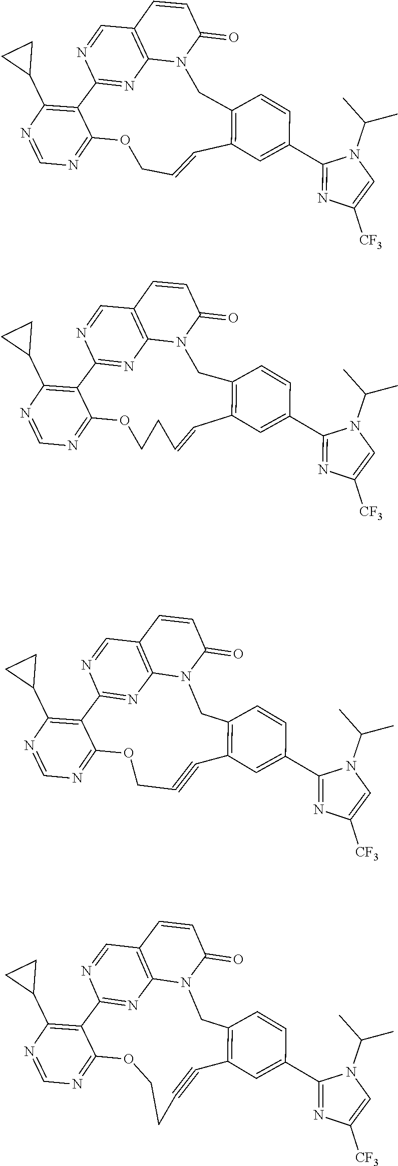 [SMILES: CC(C)n1cc(C(F)(F)F)nc1-c1ccc2c(c1)/C=C/COc1ncnc(C3CC3)c1-c1ncc3ccc(=O)n(c3n1)C2 ; CC(C)n1cc(C(F)(F)F)nc1-c1ccc2c(c1)/C=C/CCOc1ncnc(C3CC3)c1-c1ncc3ccc(=O)n(c3n1)C2 ; CC(C)n1cc(C(F)(F)F)nc1-c1ccc2c(c1)C#CCOc1ncnc(C3CC3)c1-c1ncc3ccc(=O)n(c3n1)C2 ; CC(C)n1cc(C(F)(F)F)nc1-c1ccc2c(c1)C#CCCOc1ncnc(C3CC3)c1-c1ncc3ccc(=O)n(c3n1)C2]

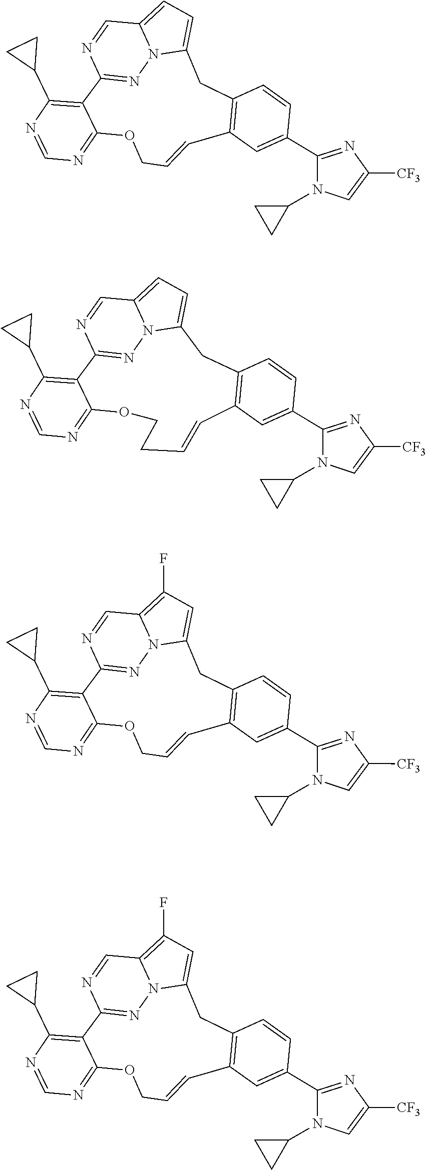 [SMILES: FC(F)(F)c1cn(C2CC2)c(-c2ccc3c(c2)/C=C/COc2ncnc(C4CC4)c2-c2ncc4ccc(n4n2)C3)n1 ; FC(F)(F)c1cn(C2CC2)c(-c2ccc3c(c2)/C=C/CCOc2ncnc(C4CC4)c2-c2ncc4ccc(n4n2)C3)n1 ; Fc1cc2n3nc(ncc13)-c1c(ncnc1C1CC1)OC/C=C/c1cc(-c3nc(C(F)(F)F)cn3C3CC3)ccc1C2 ; Fc1cc2n3nc(ncc13)-c1c(ncnc1C1CC1)OC/C=C/c1cc(-c3nc(C(F)(F)F)cn3C3CC3)ccc1C2]

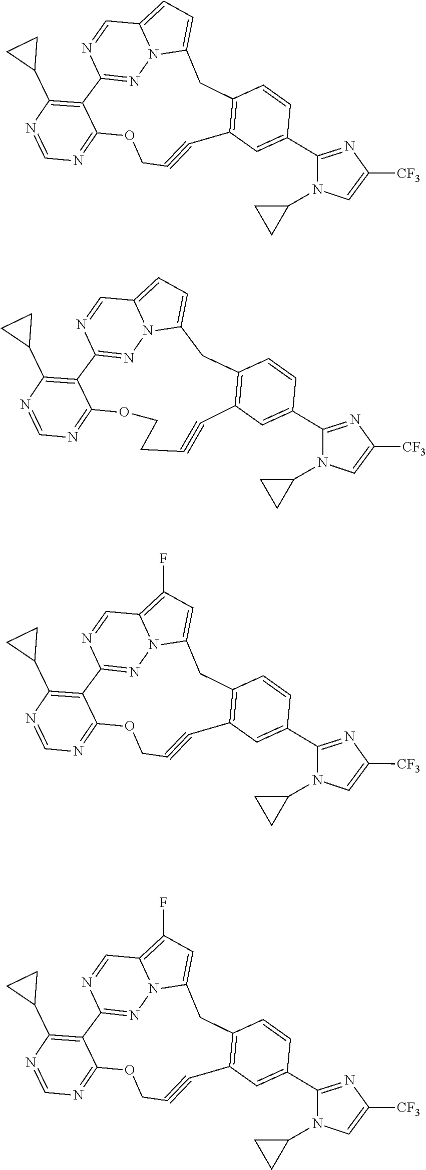 [SMILES: FC(F)(F)c1cn(C2CC2)c(-c2ccc3c(c2)C#CCOc2ncnc(C4CC4)c2-c2ncc4ccc(n4n2)C3)n1 ; FC(F)(F)c1cn(C2CC2)c(-c2ccc3c(c2)C#CCCOc2ncnc(C4CC4)c2-c2ncc4ccc(n4n2)C3)n1 ; Fc1cc2n3nc(ncc13)-c1c(ncnc1C1CC1)OCC#Cc1cc(-c3nc(C(F)(F)F)cn3C3CC3)ccc1C2 ; Fc1cc2n3nc(ncc13)-c1c(ncnc1C1CC1)OCC#Cc1cc(-c3nc(C(F)(F)F)cn3C3CC3)ccc1C2]

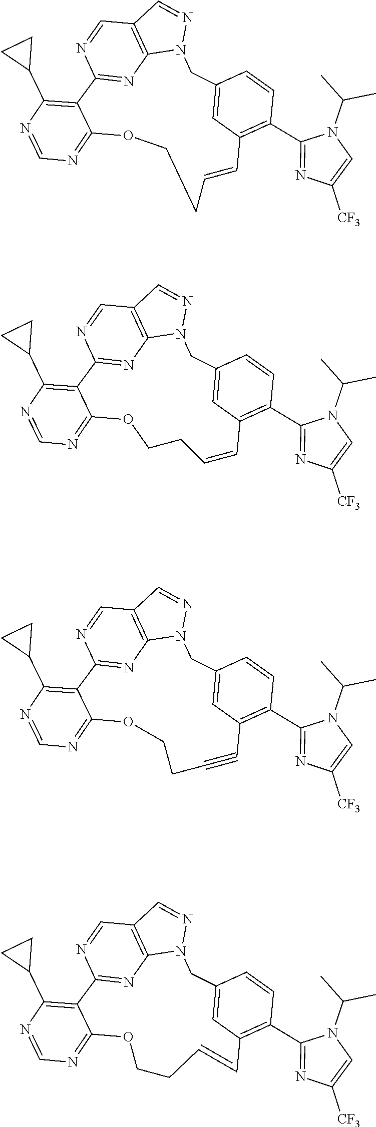 [SMILES: CC(C)n1cc(C(F)(F)F)nc1-c1ccc2cc1/C=C/CCOc1ncnc(C3CC3)c1-c1ncc3cnn(c3n1)C2 ; CC(C)n1cc(C(F)(F)F)nc1-c1ccc2cc1/C=C\CCOc1ncnc(C3CC3)c1-c1ncc3cnn(c3n1)C2 ; CC(C)n1cc(C(F)(F)F)nc1-c1ccc2cc1C#CCCOc1ncnc(C3CC3)c1-c1ncc3cnn(c3n1)C2 ; CC(C)n1cc(C(F)(F)F)nc1-c1ccc2cc1/C=C/CCOc1ncnc(C3CC3)c1-c1ncc3cnn(c3n1)C2]

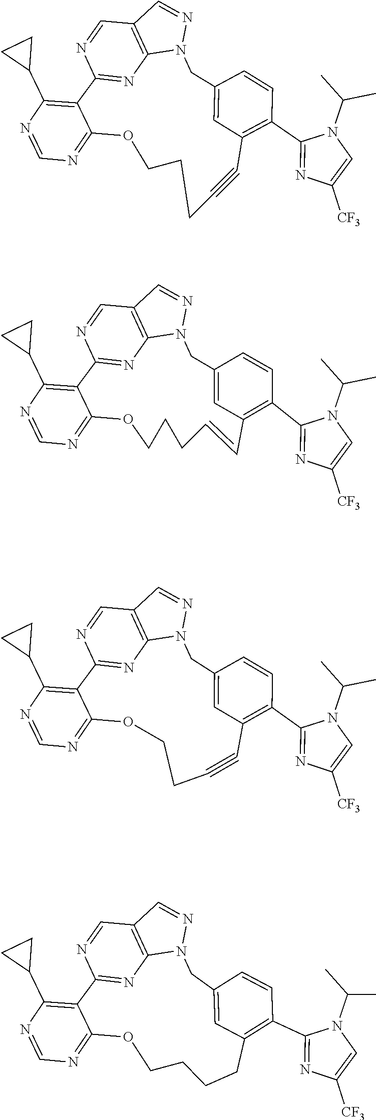 [SMILES: CC(C)n1cc(C(F)(F)F)nc1-c1ccc2cc1C#CCCCOc1ncnc(C3CC3)c1-c1ncc3cnn(c3n1)C2 ; CC(C)n1cc(C(F)(F)F)nc1-c1ccc2cc1/C=C/CCCOc1ncnc(C3CC3)c1-c1ncc3cnn(c3n1)C2 ; CC(C)n1cc(C(F)(F)F)nc1-c1ccc2cc1C#CCCOc1ncnc(C3CC3)c1-c1ncc3cnn(c3n1)C2 ; CC(C)n1cc(C(F)(F)F)nc1-c1ccc2cc1CCCCOc1ncnc(C3CC3)c1-c1ncc3cnn(c3n1)C2]

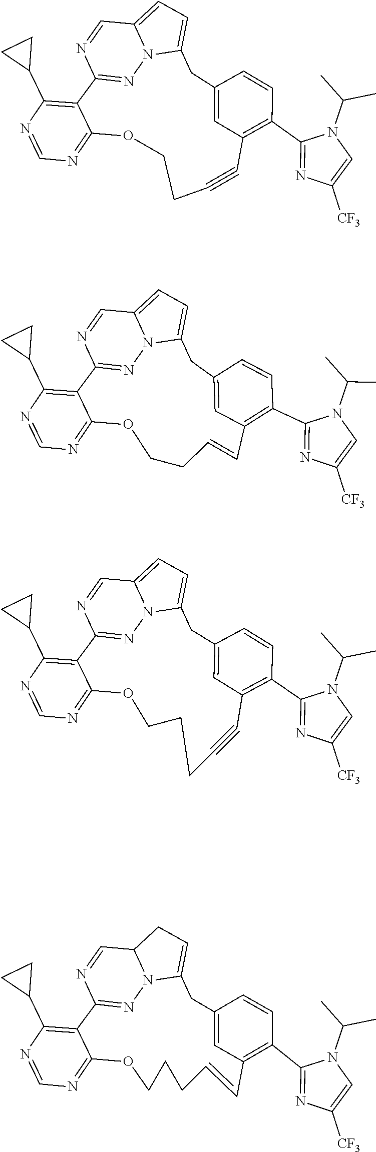 [SMILES: CC(C)n1cc(C(F)(F)F)nc1-c1ccc2cc1C#CCCOc1ncnc(C3CC3)c1-c1ncc3ccc(n3n1)C2 ; CC(C)n1cc(C(F)(F)F)nc1-c1ccc2cc1/C=C/CCOc1ncnc(C3CC3)c1-c1ncc3ccc(n3n1)C2 ; CC(C)n1cc(C(F)(F)F)nc1-c1ccc2cc1C#CCCCOc1ncnc(C3CC3)c1-c1ncc3ccc(n3n1)C2 ; CC(C)n1cc(C(F)(F)F)nc1-c1ccc2cc1/C=C/CCCOc1ncnc(C3CC3)c1C1=NN3C(=CCC3C=N1)C2]

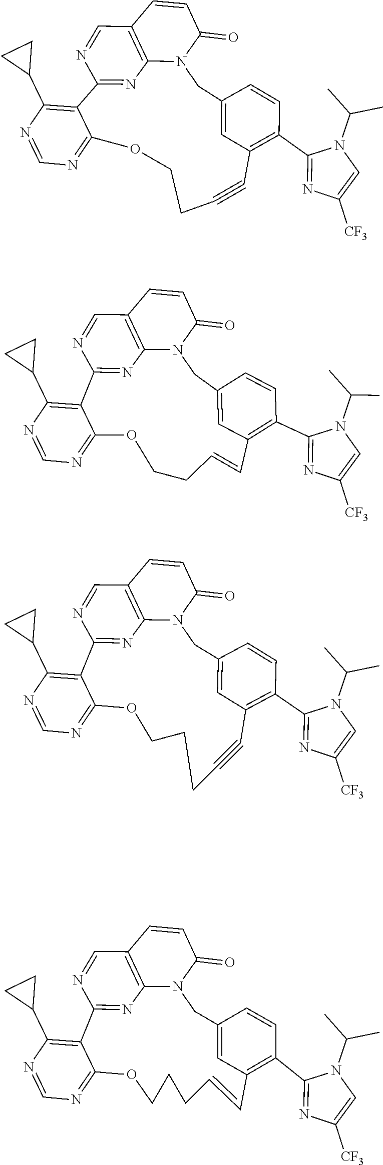 [SMILES: CC(C)n1cc(C(F)(F)F)nc1-c1ccc2cc1C#CCCOc1ncnc(C3CC3)c1-c1ncc3ccc(=O)n(c3n1)C2 ; CC(C)n1cc(C(F)(F)F)nc1-c1ccc2cc1/C=C/CCOc1ncnc(C3CC3)c1-c1ncc3ccc(=O)n(c3n1)C2 ; CC(C)n1cc(C(F)(F)F)nc1-c1ccc2cc1C#CCCCOc1ncnc(C3CC3)c1-c1ncc3ccc(=O)n(c3n1)C2 ; CC(C)n1cc(C(F)(F)F)nc1-c1ccc2cc1/C=C/CCCOc1ncnc(C3CC3)c1-c1ncc3ccc(=O)n(c3n1)C2]

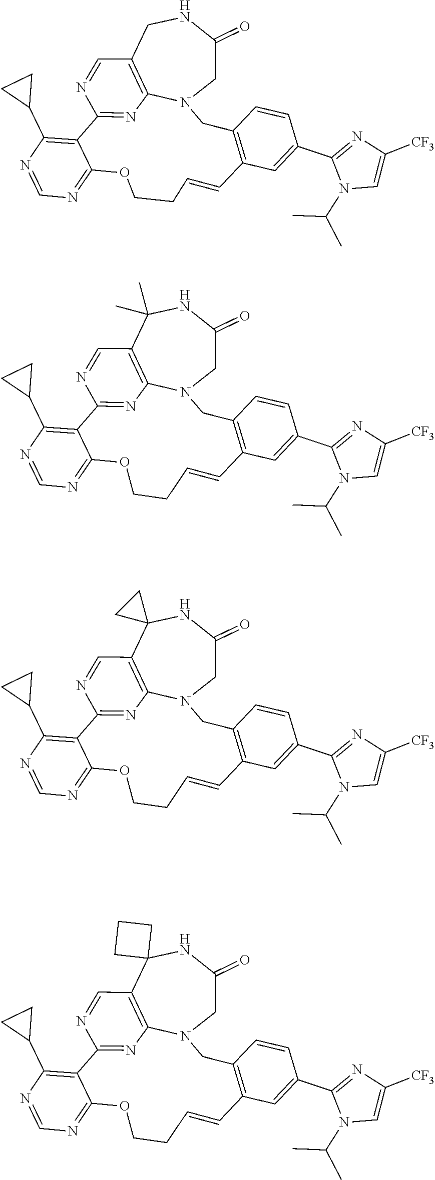 [SMILES: CC(C)n1cc(C(F)(F)F)nc1-c1ccc2c(c1)/C=C/CCOc1ncnc(C3CC3)c1-c1ncc3c(n1)N(CC(=O)NC3)C2 ; CC(C)n1cc(C(F)(F)F)nc1-c1ccc2c(c1)/C=C/CCOc1ncnc(C3CC3)c1-c1ncc3c(n1)N(CC(=O)NC3(C)C)C2 ; CC(C)n1cc(C(F)(F)F)nc1-c1ccc2c(c1)/C=C/CCOc1ncnc(C3CC3)c1-c1ncc3c(n1)N(CC(=O)NC31CC1)C2 ; CC(C)n1cc(C(F)(F)F)nc1-c1ccc2c(c1)/C=C/CCOc1ncnc(C3CC3)c1-c1ncc3c(n1)N(CC(=O)NC31CCC1)C2]

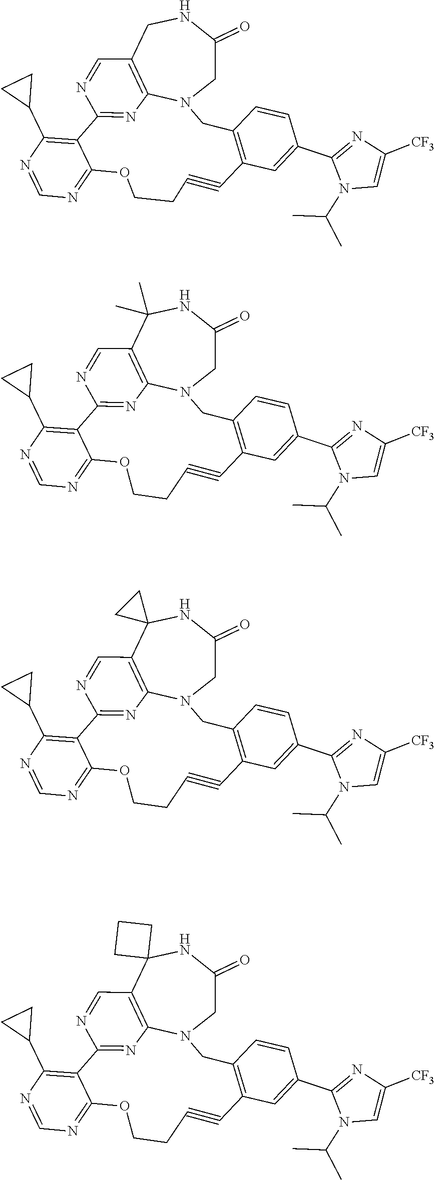 [SMILES: CC(C)n1cc(C(F)(F)F)nc1-c1ccc2c(c1)C#CCCOc1ncnc(C3CC3)c1-c1ncc3c(n1)N(CC(=O)NC3)C2 ; CC(C)n1cc(C(F)(F)F)nc1-c1ccc2c(c1)C#CCCOc1ncnc(C3CC3)c1-c1ncc3c(n1)N(CC(=O)NC3(C)C)C2 ; CC(C)n1cc(C(F)(F)F)nc1-c1ccc2c(c1)C#CCCOc1ncnc(C3CC3)c1-c1ncc3c(n1)N(CC(=O)NC31CC1)C2 ; CC(C)n1cc(C(F)(F)F)nc1-c1ccc2c(c1)C#CCCOc1ncnc(C3CC3)c1-c1ncc3c(n1)N(CC(=O)NC31CCC1)C2]

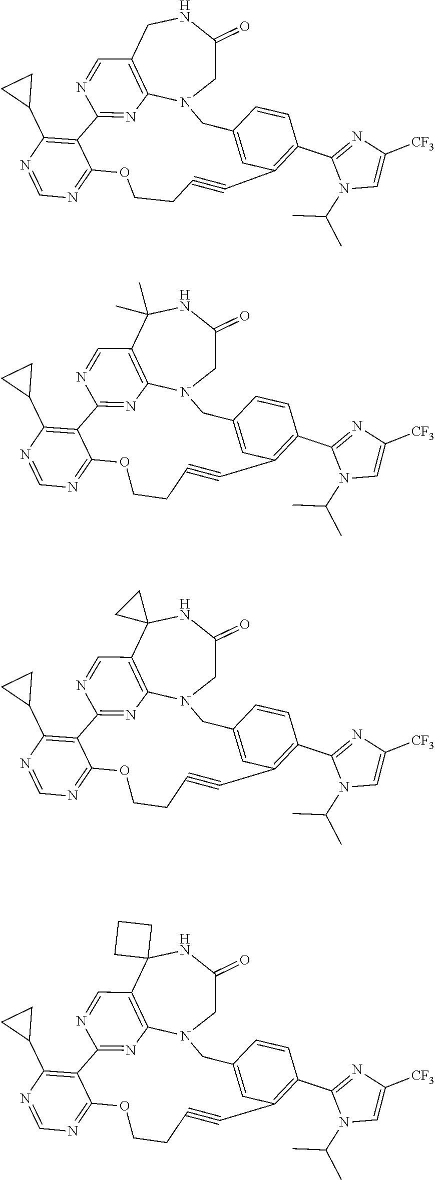 [SMILES: CC(C)n1cc(C(F)(F)F)nc1-c1ccc2cc1C#CCCOc1ncnc(C3CC3)c1-c1ncc3c(n1)N(CC(=O)NC3)C2 ; CC(C)n1cc(C(F)(F)F)nc1-c1ccc2cc1C#CCCOc1ncnc(C3CC3)c1-c1ncc3c(n1)N(CC(=O)NC3(C)C)C2 ; CC(C)n1cc(C(F)(F)F)nc1-c1ccc2cc1C#CCCOc1ncnc(C3CC3)c1-c1ncc3c(n1)N(CC(=O)NC31CC1)C2 ; CC(C)n1cc(C(F)(F)F)nc1-c1ccc2cc1C#CCCOc1ncnc(C3CC3)c1-c1ncc3c(n1)N(CC(=O)NC31CCC1)C2]

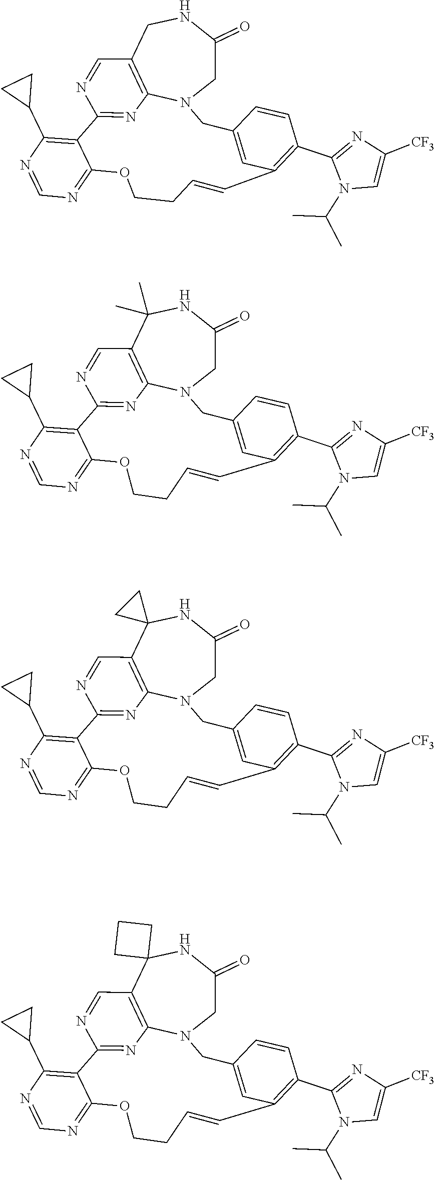 [SMILES: CC(C)n1cc(C(F)(F)F)nc1-c1ccc2cc1/C=C/CCOc1ncnc(C3CC3)c1-c1ncc3c(n1)N(CC(=O)NC3)C2 ; CC(C)n1cc(C(F)(F)F)nc1-c1ccc2cc1/C=C/CCOc1ncnc(C3CC3)c1-c1ncc3c(n1)N(CC(=O)NC3(C)C)C2 ; CC(C)n1cc(C(F)(F)F)nc1-c1ccc2cc1/C=C/CCOc1ncnc(C3CC3)c1-c1ncc3c(n1)N(CC(=O)NC31CC1)C2 ; CC(C)n1cc(C(F)(F)F)nc1-c1ccc2cc1/C=C/CCOc1ncnc(C3CC3)c1-c1ncc3c(n1)N(CC(=O)NC31CCC1)C2]

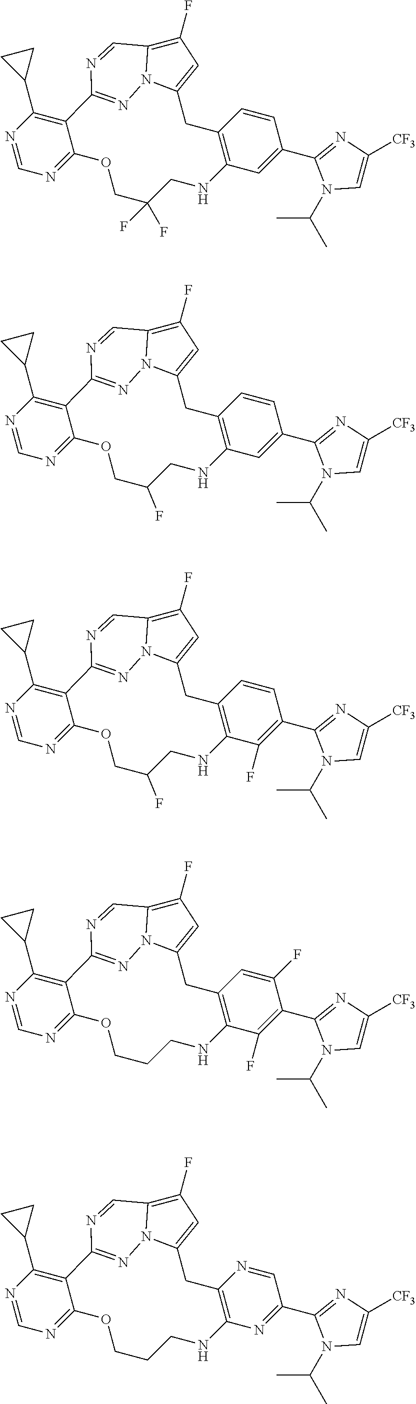 [SMILES: CC(C)n1cc(C(F)(F)F)nc1-c1ccc2c(c1)NCC(F)(F)COc1ncnc(C3CC3)c1-c1ncc3c(F)cc(n3n1)C2 ; CC(C)n1cc(C(F)(F)F)nc1-c1ccc2c(c1)NCC(F)COc1ncnc(C3CC3)c1-c1ncc3c(F)cc(n3n1)C2 ; CC(C)n1cc(C(F)(F)F)nc1-c1ccc2c(c1F)NCC(F)COc1ncnc(C3CC3)c1-c1ncc3c(F)cc(n3n1)C2 ; CC(C)n1cc(C(F)(F)F)nc1-c1c(F)cc2c(c1F)NCCCOc1ncnc(C3CC3)c1-c1ncc3c(F)cc(n3n1)C2 ; CC(C)n1cc(C(F)(F)F)nc1-c1cnc2c(n1)NCCCOc1ncnc(C3CC3)c1-c1ncc3c(F)cc(n3n1)C2]

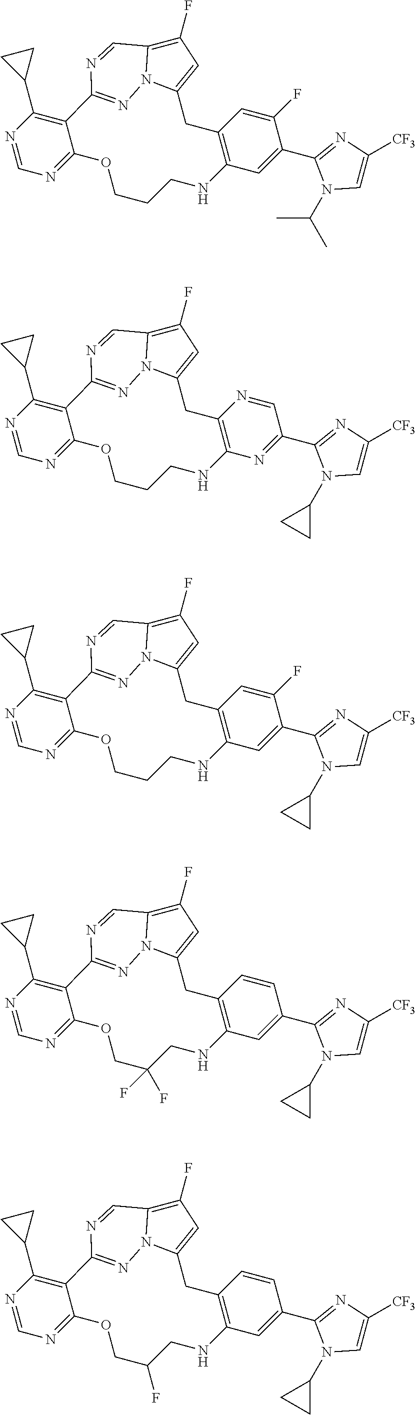 [SMILES: CC(C)n1cc(C(F)(F)F)nc1-c1cc2c(cc1F)Cc1cc(F)c3cnc(nn13)-c1c(ncnc1C1CC1)OCCCN2 ; Fc1cc2n3nc(ncc13)-c1c(ncnc1C1CC1)OCCCNc1nc(-c3nc(C(F)(F)F)cn3C3CC3)cnc1C2 ; Fc1cc2c(cc1-c1nc(C(F)(F)F)cn1C1CC1)NCCCOc1ncnc(C3CC3)c1-c1ncc3c(F)cc(n3n1)C2 ; Fc1cc2n3nc(ncc13)-c1c(ncnc1C1CC1)OCC(F)(F)CNc1cc(-c3nc(C(F)(F)F)cn3C3CC3)ccc1C2 ; Fc1cc2n3nc(ncc13)-c1c(ncnc1C1CC1)OCC(F)CNc1cc(-c3nc(C(F)(F)F)cn3C3CC3)ccc1C2]

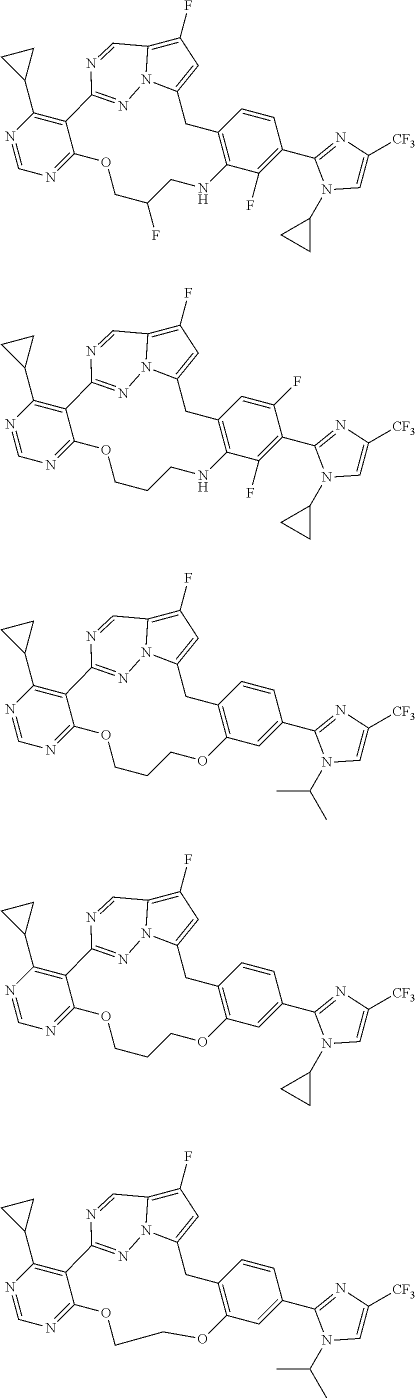 [SMILES: Fc1c(-c2nc(C(F)(F)F)cn2C2CC2)ccc2c1NCC(F)COc1ncnc(C3CC3)c1-c1ncc3c(F)cc(n3n1)C2 ; Fc1cc2c(c(F)c1-c1nc(C(F)(F)F)cn1C1CC1)NCCCOc1ncnc(C3CC3)c1-c1ncc3c(F)cc(n3n1)C2 ; CC(C)n1cc(C(F)(F)F)nc1-c1ccc2c(c1)OCCCOc1ncnc(C3CC3)c1-c1ncc3c(F)cc(n3n1)C2 ; Fc1cc2n3nc(ncc13)-c1c(ncnc1C1CC1)OCCCOc1cc(-c3nc(C(F)(F)F)cn3C3CC3)ccc1C2 ; CC(C)n1cc(C(F)(F)F)nc1-c1ccc2c(c1)OCCOc1ncnc(C3CC3)c1-c1ncc3c(F)cc(n3n1)C2]

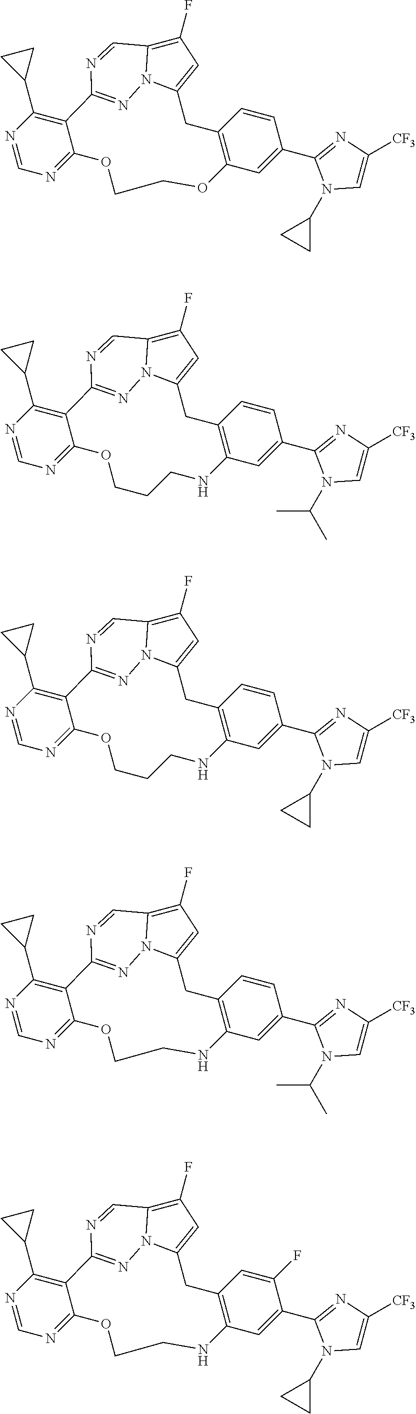 [SMILES: Fc1cc2n3nc(ncc13)-c1c(ncnc1C1CC1)OCCOc1cc(-c3nc(C(F)(F)F)cn3C3CC3)ccc1C2 ; CC(C)n1cc(C(F)(F)F)nc1-c1ccc2c(c1)NCCCOc1ncnc(C3CC3)c1-c1ncc3c(F)cc(n3n1)C2 ; Fc1cc2n3nc(ncc13)-c1c(ncnc1C1CC1)OCCCNc1cc(-c3nc(C(F)(F)F)cn3C3CC3)ccc1C2 ; CC(C)n1cc(C(F)(F)F)nc1-c1ccc2c(c1)NCCOc1ncnc(C3CC3)c1-c1ncc3c(F)cc(n3n1)C2 ; Fc1cc2c(cc1-c1nc(C(F)(F)F)cn1C1CC1)NCCOc1ncnc(C3CC3)c1-c1ncc3c(F)cc(n3n1)C2]

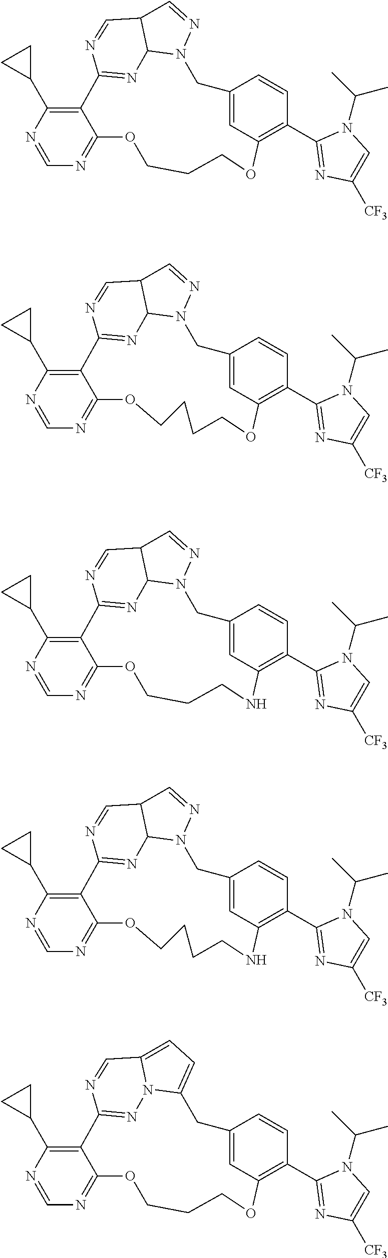 [SMILES: CC(C)n1cc(C(F)(F)F)nc1-c1ccc2cc1OCCCOc1ncnc(C3CC3)c1C1=NC3C(C=N1)C=NN3C2 ; CC(C)n1cc(C(F)(F)F)nc1-c1ccc2cc1OCCCCOc1ncnc(C3CC3)c1C1=NC3C(C=N1)C=NN3C2 ; CC(C)n1cc(C(F)(F)F)nc1-c1ccc2cc1NCCCOc1ncnc(C3CC3)c1C1=NC3C(C=N1)C=NN3C2 ; CC(C)n1cc(C(F)(F)F)nc1-c1ccc2cc1NCCCCOc1ncnc(C3CC3)c1C1=NC3C(C=N1)C=NN3C2 ; CC(C)n1cc(C(F)(F)F)nc1-c1ccc2cc1OCCCOc1ncnc(C3CC3)c1-c1ncc3ccc(n3n1)C2]

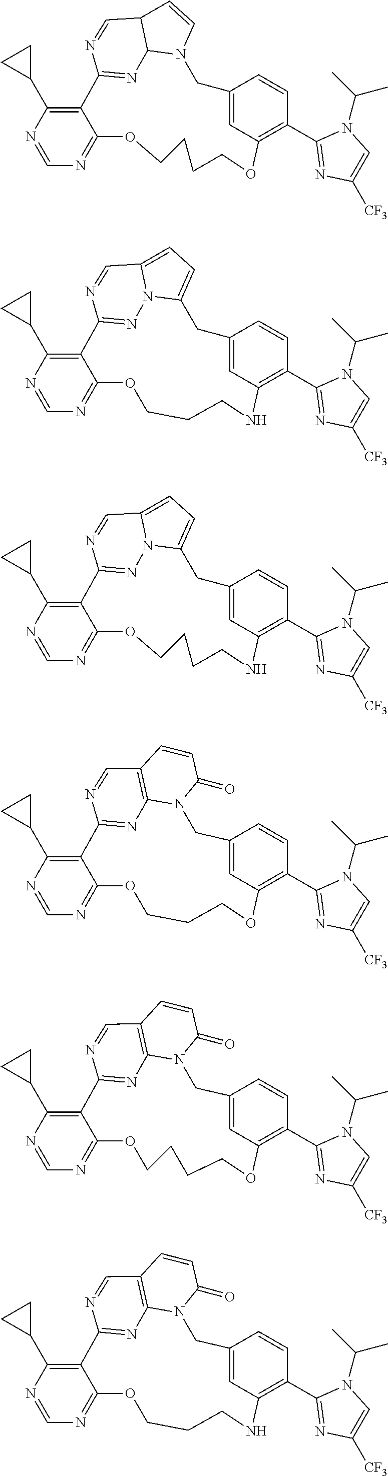 [SMILES: CC(C)n1cc(C(F)(F)F)nc1-c1ccc2cc1OCCCCOc1ncnc(C3CC3)c1C1=NC3C(C=CN3C2)C=N1 ; CC(C)n1cc(C(F)(F)F)nc1-c1ccc2cc1NCCCOc1ncnc(C3CC3)c1-c1ncc3ccc(n3n1)C2 ; CC(C)n1cc(C(F)(F)F)nc1-c1ccc2cc1NCCCCOc1ncnc(C3CC3)c1-c1ncc3ccc(n3n1)C2 ; CC(C)n1cc(C(F)(F)F)nc1-c1ccc2cc1OCCCOc1ncnc(C3CC3)c1-c1ncc3ccc(=O)n(c3n1)C2 ; CC(C)n1cc(C(F)(F)F)nc1-c1ccc2cc1OCCCCOc1ncnc(C3CC3)c1-c1ncc3ccc(=O)n(c3n1)C2 ; CC(C)n1cc(C(F)(F)F)nc1-c1ccc2cc1NCCCOc1ncnc(C3CC3)c1-c1ncc3ccc(=O)n(c3n1)C2]

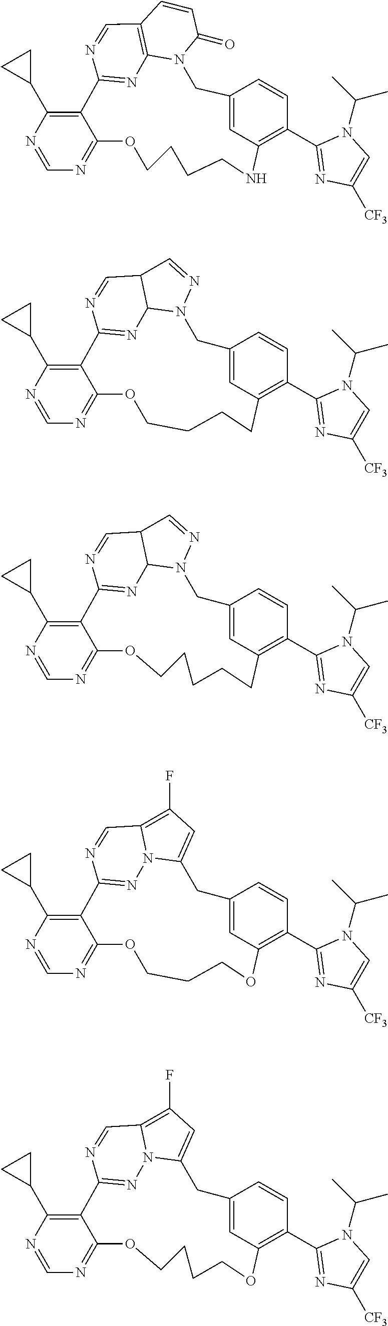 [SMILES: CC(C)n1cc(C(F)(F)F)nc1-c1ccc2cc1NCCCCOc1ncnc(C3CC3)c1-c1ncc3ccc(=O)n(c3n1)C2 ; CC(C)n1cc(C(F)(F)F)nc1-c1ccc2cc1CCCCOc1ncnc(C3CC3)c1C1=NC3C(C=N1)C=NN3C2 ; CC(C)n1cc(C(F)(F)F)nc1-c1ccc2cc1CCCCCOc1ncnc(C3CC3)c1C1=NC3C(C=N1)C=NN3C2 ; CC(C)n1cc(C(F)(F)F)nc1-c1ccc2cc1OCCCOc1ncnc(C3CC3)c1-c1ncc3c(F)cc(n3n1)C2 ; CC(C)n1cc(C(F)(F)F)nc1-c1ccc2cc1OCCCCOc1ncnc(C3CC3)c1-c1ncc3c(F)cc(n3n1)C2]

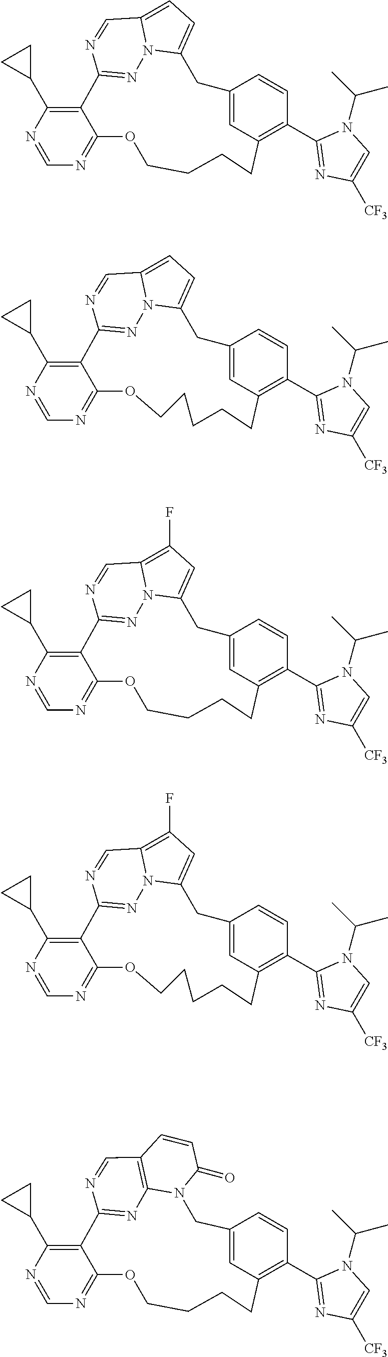 [SMILES: CC(C)n1cc(C(F)(F)F)nc1-c1ccc2cc1CCCCOc1ncnc(C3CC3)c1-c1ncc3ccc(n3n1)C2 ; CC(C)n1cc(C(F)(F)F)nc1-c1ccc2cc1CCCCCOc1ncnc(C3CC3)c1-c1ncc3ccc(n3n1)C2 ; CC(C)n1cc(C(F)(F)F)nc1-c1ccc2cc1CCCCOc1ncnc(C3CC3)c1-c1ncc3c(F)cc(n3n1)C2 ; CC(C)n1cc(C(F)(F)F)nc1-c1ccc2cc1CCCCCOc1ncnc(C3CC3)c1-c1ncc3c(F)cc(n3n1)C2 ; CC(C)n1cc(C(F)(F)F)nc1-c1ccc2cc1CCCCOc1ncnc(C3CC3)c1-c1ncc3ccc(=O)n(c3n1)C2]

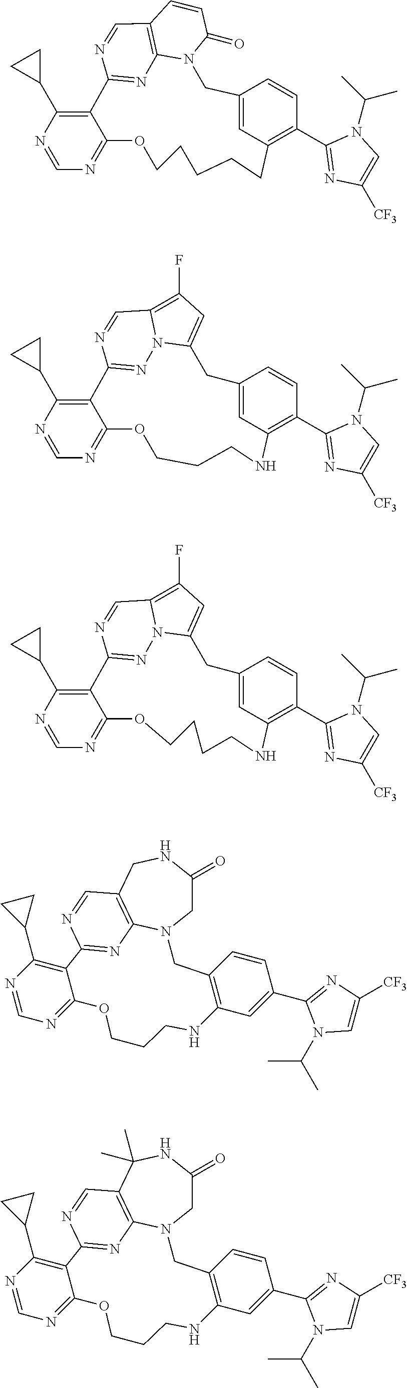 [SMILES: CC(C)n1cc(C(F)(F)F)nc1-c1ccc2cc1CCCCCOc1ncnc(C3CC3)c1-c1ncc3ccc(=O)n(c3n1)C2 ; CC(C)n1cc(C(F)(F)F)nc1-c1ccc2cc1NCCCOc1ncnc(C3CC3)c1-c1ncc3c(F)cc(n3n1)C2 ; CC(C)n1cc(C(F)(F)F)nc1-c1ccc2cc1NCCCCOc1ncnc(C3CC3)c1-c1ncc3c(F)cc(n3n1)C2 ; CC(C)n1cc(C(F)(F)F)nc1-c1ccc2c(c1)NCCCOc1ncnc(C3CC3)c1-c1ncc3c(n1)N(CC(=O)NC3)C2 ; CC(C)n1cc(C(F)(F)F)nc1-c1ccc2c(c1)NCCCOc1ncnc(C3CC3)c1-c1ncc3c(n1)N(CC(=O)NC3(C)C)C2]

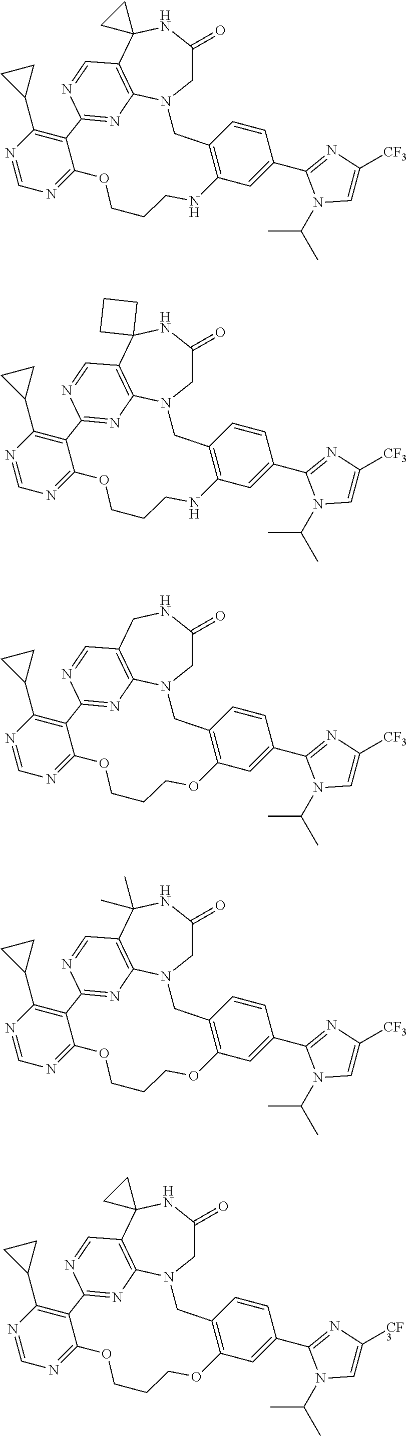 [SMILES: CC(C)n1cc(C(F)(F)F)nc1-c1ccc2c(c1)NCCCOc1ncnc(C3CC3)c1-c1ncc3c(n1)N(CC(=O)NC31CC1)C2 ; CC(C)n1cc(C(F)(F)F)nc1-c1ccc2c(c1)NCCCOc1ncnc(C3CC3)c1-c1ncc3c(n1)N(CC(=O)NC31CCC1)C2 ; CC(C)n1cc(C(F)(F)F)nc1-c1ccc2c(c1)OCCCOc1ncnc(C3CC3)c1-c1ncc3c(n1)N(CC(=O)NC3)C2 ; CC(C)n1cc(C(F)(F)F)nc1-c1ccc2c(c1)OCCCOc1ncnc(C3CC3)c1-c1ncc3c(n1)N(CC(=O)NC3(C)C)C2 ; CC(C)n1cc(C(F)(F)F)nc1-c1ccc2c(c1)OCCCOc1ncnc(C3CC3)c1-c1ncc3c(n1)N(CC(=O)NC31CC1)C2]

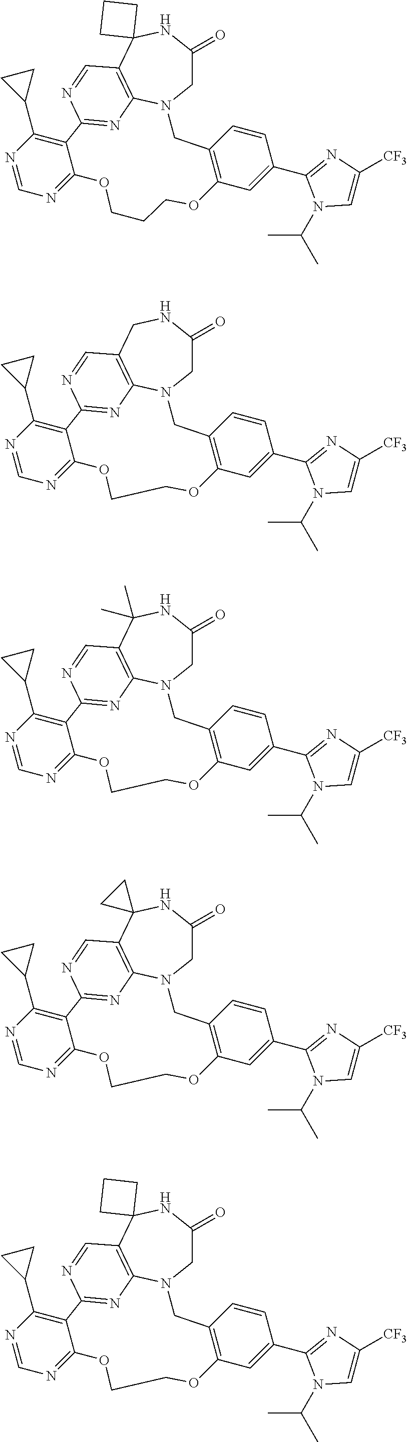 [SMILES: CC(C)n1cc(C(F)(F)F)nc1-c1ccc2c(c1)OCCCOc1ncnc(C3CC3)c1-c1ncc3c(n1)N(CC(=O)NC31CCC1)C2 ; CC(C)n1cc(C(F)(F)F)nc1-c1ccc2c(c1)OCCOc1ncnc(C3CC3)c1-c1ncc3c(n1)N(CC(=O)NC3)C2 ; CC(C)n1cc(C(F)(F)F)nc1-c1ccc2c(c1)OCCOc1ncnc(C3CC3)c1-c1ncc3c(n1)N(CC(=O)NC3(C)C)C2 ; CC(C)n1cc(C(F)(F)F)nc1-c1ccc2c(c1)OCCOc1ncnc(C3CC3)c1-c1ncc3c(n1)N(CC(=O)NC31CC1)C2 ; CC(C)n1cc(C(F)(F)F)nc1-c1ccc2c(c1)OCCOc1ncnc(C3CC3)c1-c1ncc3c(n1)N(CC(=O)NC31CCC1)C2]

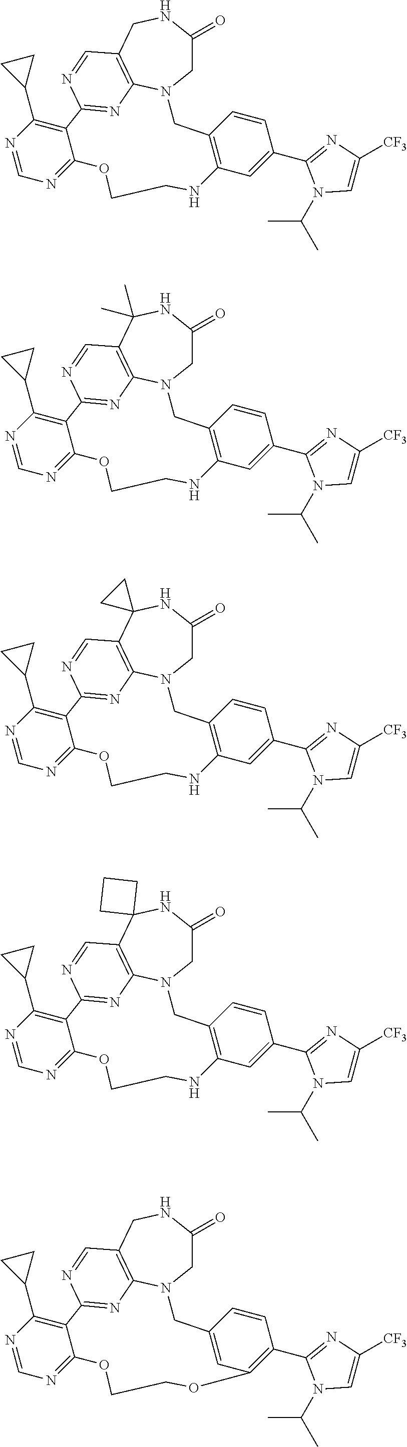 [SMILES: CC(C)n1cc(C(F)(F)F)nc1-c1ccc2c(c1)NCCOc1ncnc(C3CC3)c1-c1ncc3c(n1)N(CC(=O)NC3)C2 ; CC(C)n1cc(C(F)(F)F)nc1-c1ccc2c(c1)NCCOc1ncnc(C3CC3)c1-c1ncc3c(n1)N(CC(=O)NC3(C)C)C2 ; CC(C)n1cc(C(F)(F)F)nc1-c1ccc2c(c1)NCCOc1ncnc(C3CC3)c1-c1ncc3c(n1)N(CC(=O)NC31CC1)C2 ; CC(C)n1cc(C(F)(F)F)nc1-c1ccc2c(c1)NCCOc1ncnc(C3CC3)c1-c1ncc3c(n1)N(CC(=O)NC31CCC1)C2 ; CC(C)n1cc(C(F)(F)F)nc1-c1ccc2cc1OCCOc1ncnc(C3CC3)c1-c1ncc3c(n1)N(CC(=O)NC3)C2]

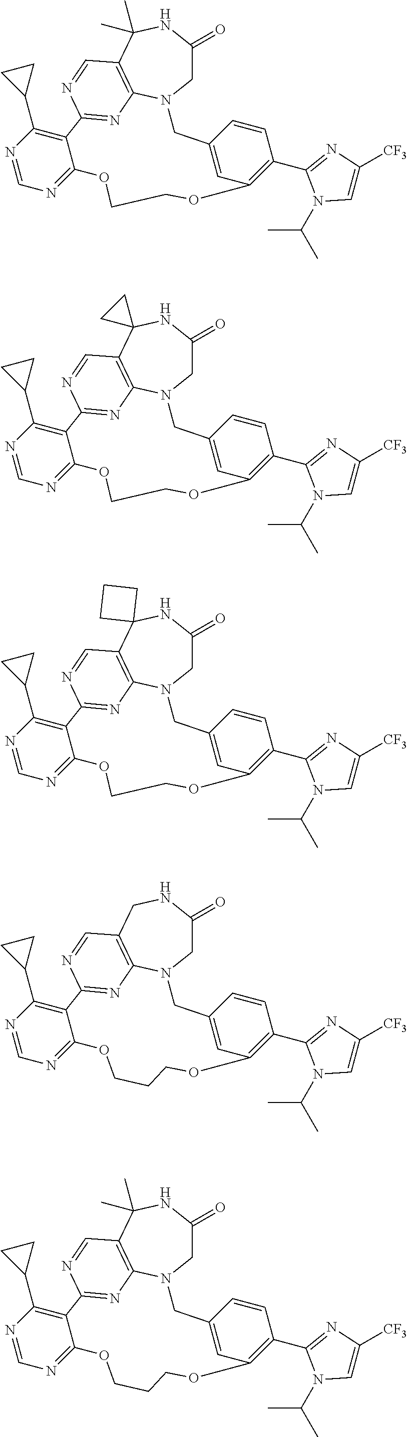 [SMILES: CC(C)n1cc(C(F)(F)F)nc1-c1ccc2cc1OCCOc1ncnc(C3CC3)c1-c1ncc3c(n1)N(CC(=O)NC3(C)C)C2 ; CC(C)n1cc(C(F)(F)F)nc1-c1ccc2cc1OCCOc1ncnc(C3CC3)c1-c1ncc3c(n1)N(CC(=O)NC31CC1)C2 ; CC(C)n1cc(C(F)(F)F)nc1-c1ccc2cc1OCCOc1ncnc(C3CC3)c1-c1ncc3c(n1)N(CC(=O)NC31CCC1)C2 ; CC(C)n1cc(C(F)(F)F)nc1-c1ccc2cc1OCCCOc1ncnc(C3CC3)c1-c1ncc3c(n1)N(CC(=O)NC3)C2 ; CC(C)n1cc(C(F)(F)F)nc1-c1ccc2cc1OCCCOc1ncnc(C3CC3)c1-c1ncc3c(n1)N(CC(=O)NC3(C)C)C2]

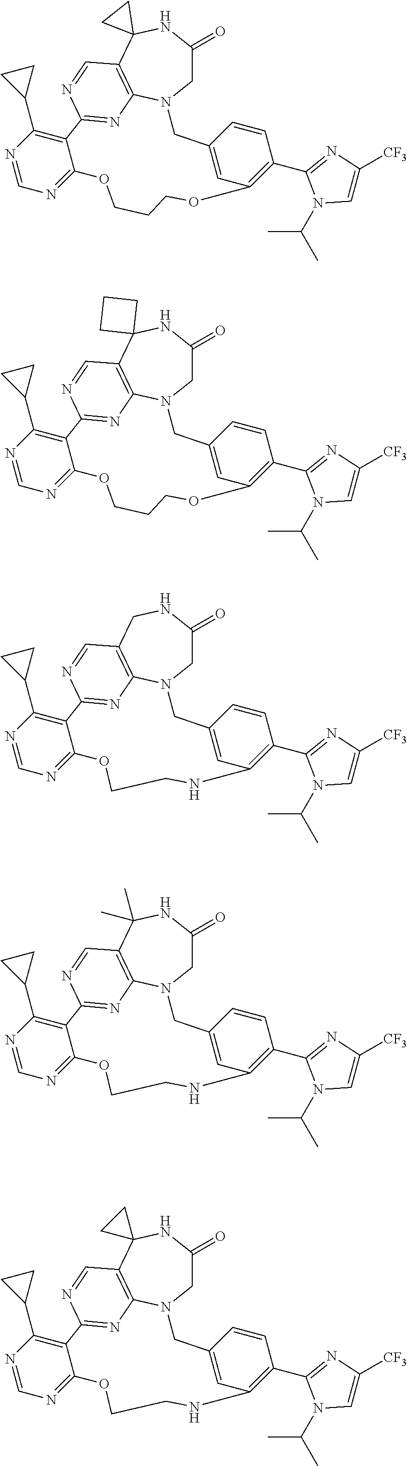 [SMILES: CC(C)n1cc(C(F)(F)F)nc1-c1ccc2cc1OCCCOc1ncnc(C3CC3)c1-c1ncc3c(n1)N(CC(=O)NC31CC1)C2 ; CC(C)n1cc(C(F)(F)F)nc1-c1ccc2cc1OCCCOc1ncnc(C3CC3)c1-c1ncc3c(n1)N(CC(=O)NC31CCC1)C2 ; CC(C)n1cc(C(F)(F)F)nc1-c1ccc2cc1NCCOc1ncnc(C3CC3)c1-c1ncc3c(n1)N(CC(=O)NC3)C2 ; CC(C)n1cc(C(F)(F)F)nc1-c1ccc2cc1NCCOc1ncnc(C3CC3)c1-c1ncc3c(n1)N(CC(=O)NC3(C)C)C2 ; CC(C)n1cc(C(F)(F)F)nc1-c1ccc2cc1NCCOc1ncnc(C3CC3)c1-c1ncc3c(n1)N(CC(=O)NC31CC1)C2]

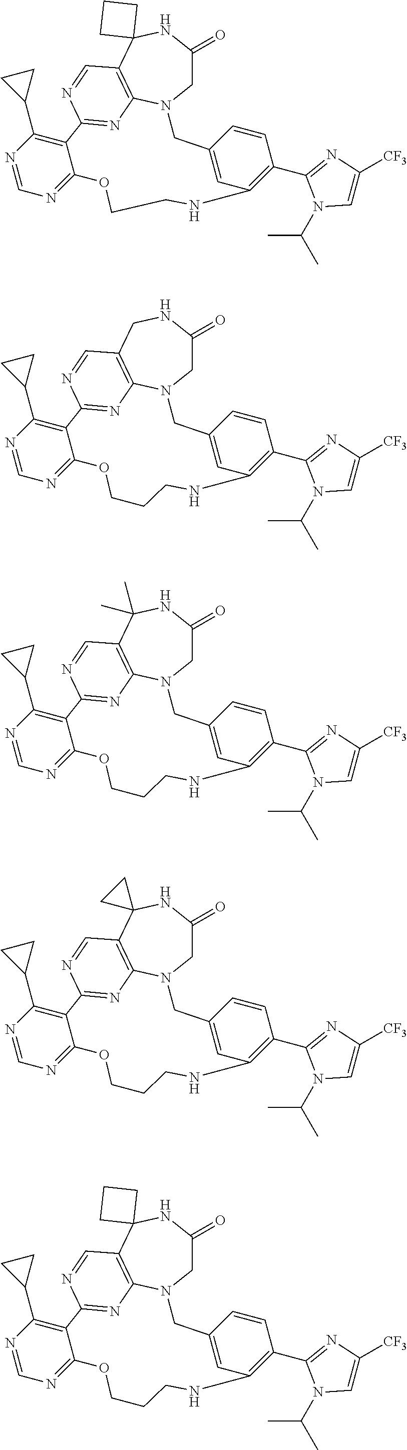 [SMILES: CC(C)n1cc(C(F)(F)F)nc1-c1ccc2cc1NCCOc1ncnc(C3CC3)c1-c1ncc3c(n1)N(CC(=O)NC31CCC1)C2 ; CC(C)n1cc(C(F)(F)F)nc1-c1ccc2cc1NCCCOc1ncnc(C3CC3)c1-c1ncc3c(n1)N(CC(=O)NC3)C2 ; CC(C)n1cc(C(F)(F)F)nc1-c1ccc2cc1NCCCOc1ncnc(C3CC3)c1-c1ncc3c(n1)N(CC(=O)NC3(C)C)C2 ; CC(C)n1cc(C(F)(F)F)nc1-c1ccc2cc1NCCCOc1ncnc(C3CC3)c1-c1ncc3c(n1)N(CC(=O)NC31CC1)C2 ; CC(C)n1cc(C(F)(F)F)nc1-c1ccc2cc1NCCCOc1ncnc(C3CC3)c1-c1ncc3c(n1)N(CC(=O)NC31CCC1)C2]

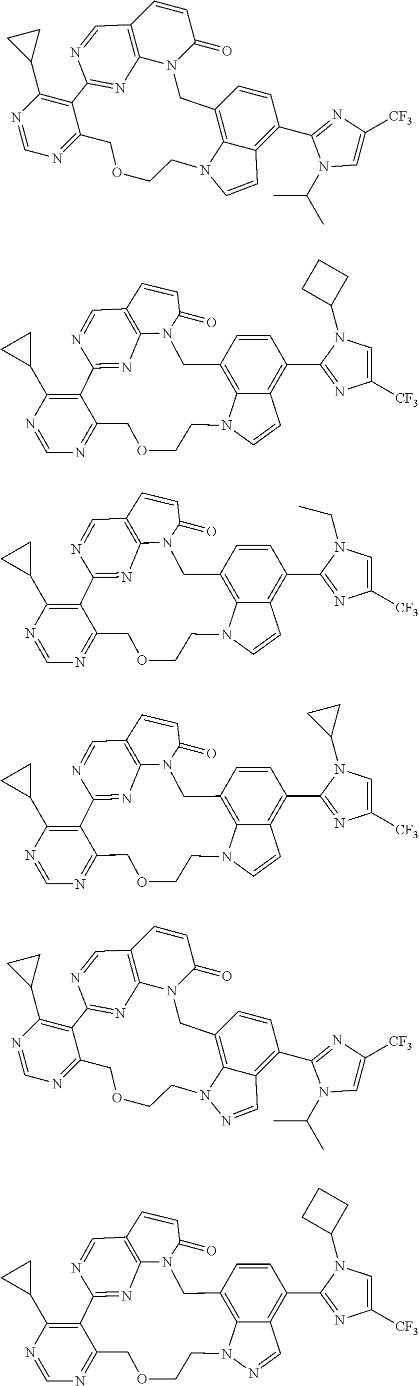

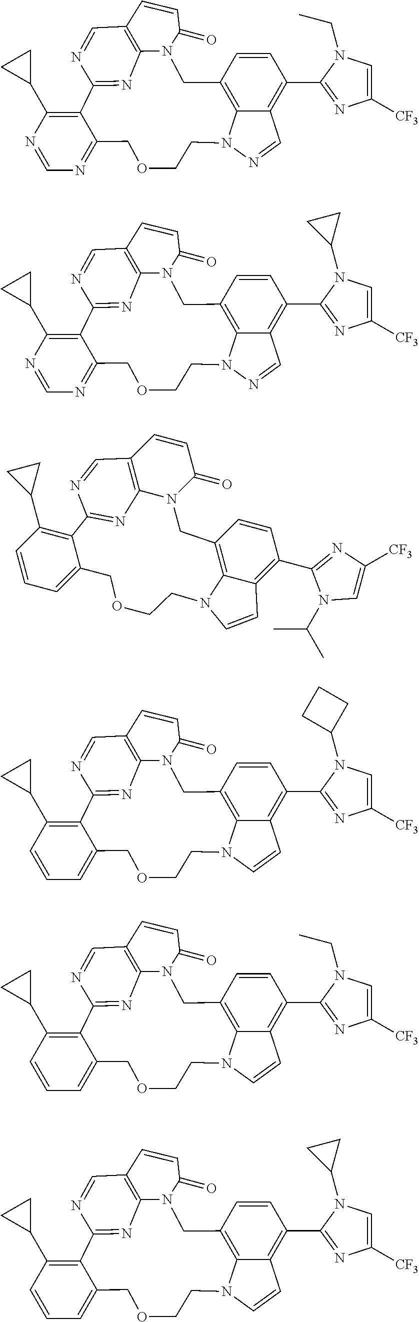

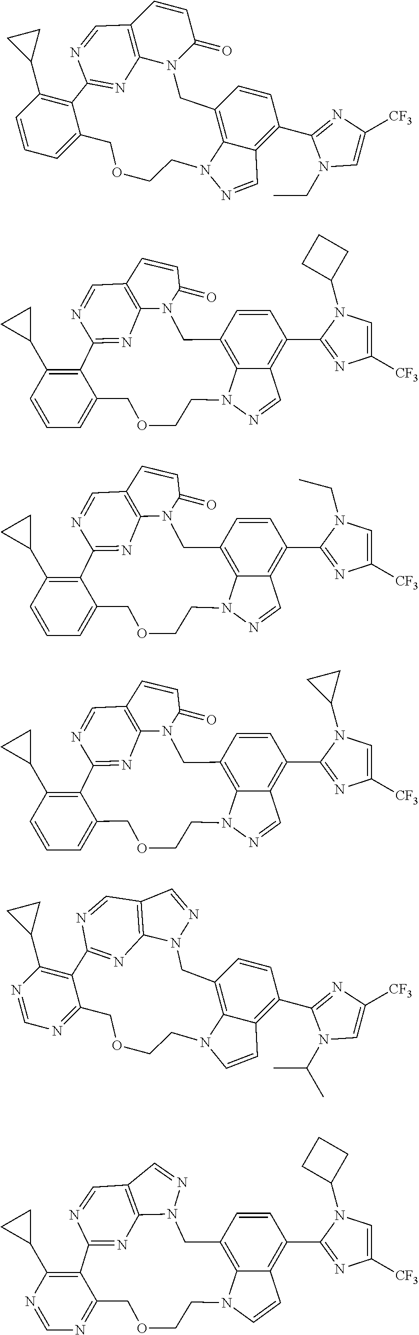

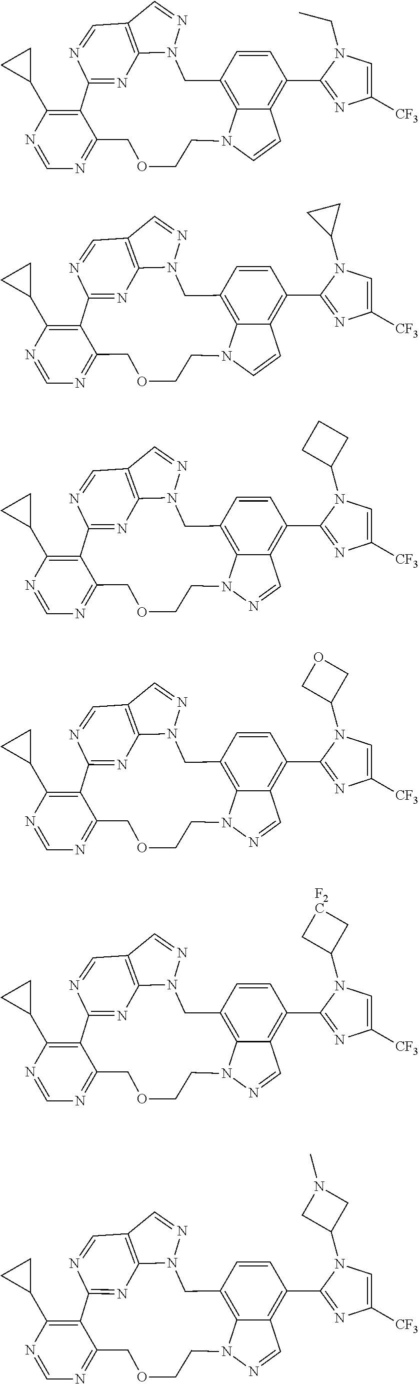 [SMILES: CCn1cc(C(F)(F)F)nc1-c1ccc2c3c1ccn3CCOCc1ncnc(C3CC3)c1-c1ncc3cnn(c3n1)C2 ; FC(F)(F)c1cn(C2CC2)c(-c2ccc3c4c2ccn4CCOCc2ncnc(C4CC4)c2-c2ncc4cnn(c4n2)C3)n1 ; FC(F)(F)c1cn(C2CCC2)c(-c2ccc3c4c2cnn4CCOCc2ncnc(C4CC4)c2-c2ncc4cnn(c4n2)C3)n1 ; FC(F)(F)c1cn(C2COC2)c(-c2ccc3c4c2cnn4CCOCc2ncnc(C4CC4)c2-c2ncc4cnn(c4n2)C3)n1 ; FC1(F)CC(n2cc(C(F)(F)F)nc2-c2ccc3c4c2cnn4CCOCc2ncnc(C4CC4)c2-c2ncc4cnn(c4n2)C3)C1 ; CN1CC(n2cc(C(F)(F)F)nc2-c2ccc3c4c2cnn4CCOCc2ncnc(C4CC4)c2-c2ncc4cnn(c4n2)C3)C1]

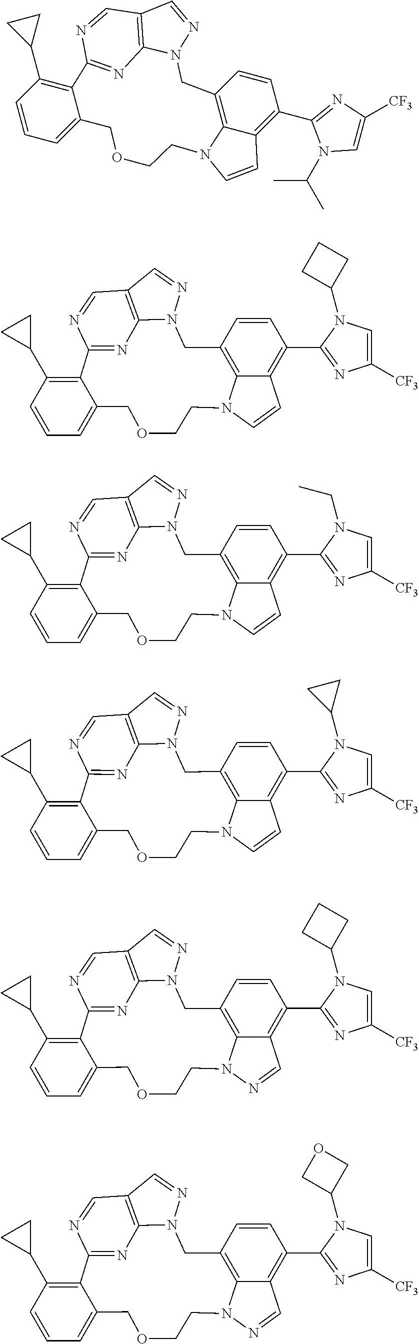

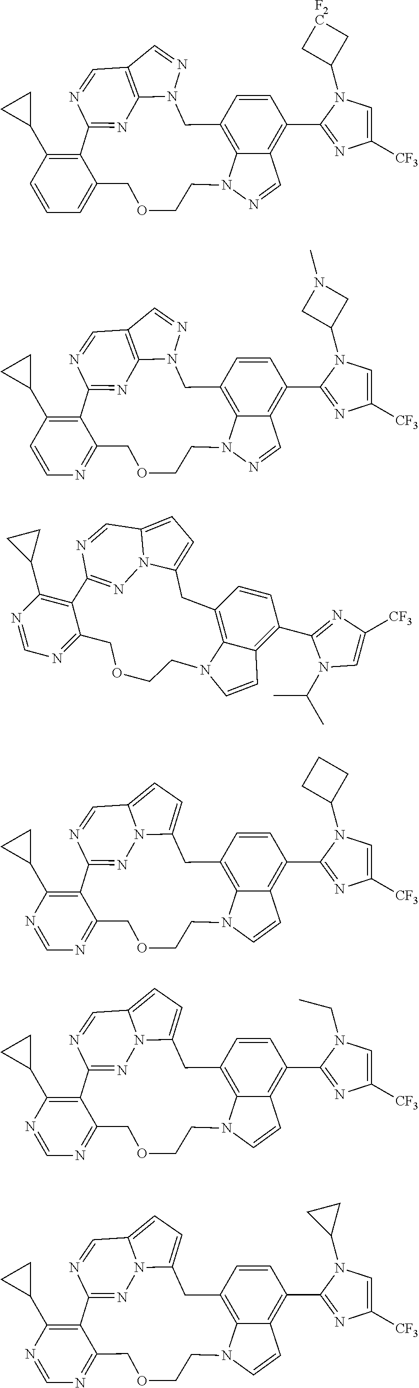

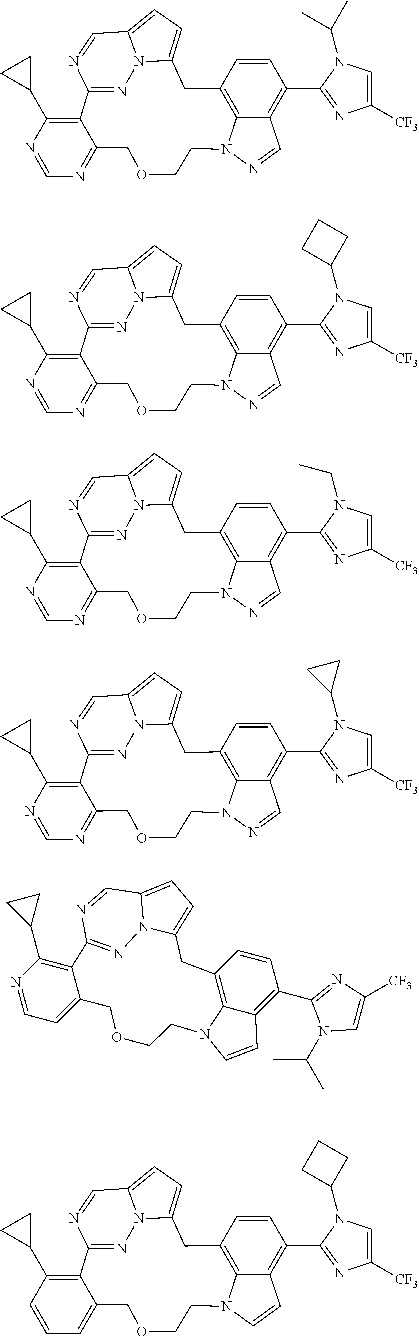

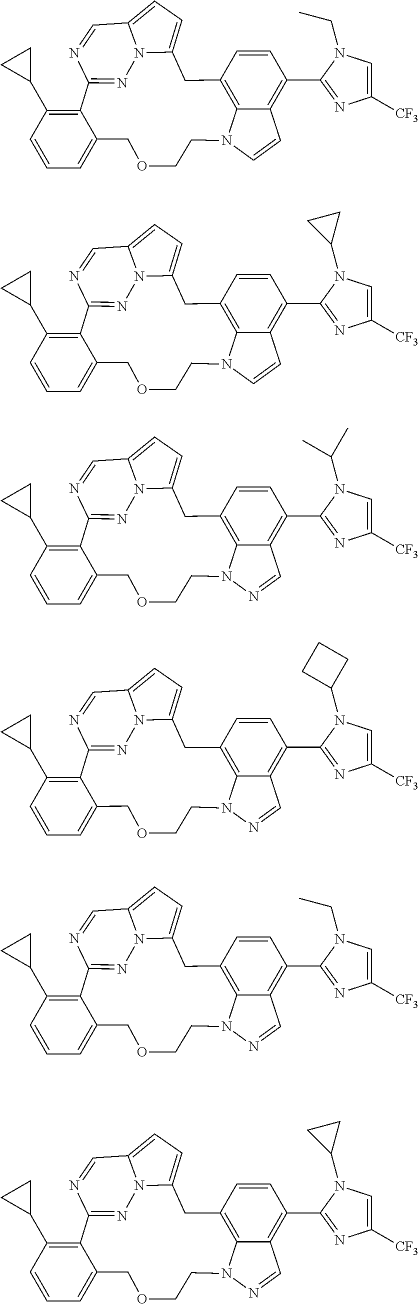 [SMILES: CCn1cc(C(F)(F)F)nc1-c1ccc2c3c1ccn3CCOCc1cccc(C3CC3)c1-c1ncc3ccc(n3n1)C2 ; FC(F)(F)c1cn(C2CC2)c(-c2ccc3c4c2ccn4CCOCc2cccc(C4CC4)c2-c2ncc4ccc(n4n2)C3)n1 ; CC(C)n1cc(C(F)(F)F)nc1-c1ccc2c3c1cnn3CCOCc1cccc(C3CC3)c1-c1ncc3ccc(n3n1)C2 ; FC(F)(F)c1cn(C2CCC2)c(-c2ccc3c4c2cnn4CCOCc2cccc(C4CC4)c2-c2ncc4ccc(n4n2)C3)n1 ; CCn1cc(C(F)(F)F)nc1-c1ccc2c3c1cnn3CCOCc1cccc(C3CC3)c1-c1ncc3ccc(n3n1)C2 ; FC(F)(F)c1cn(C2CC2)c(-c2ccc3c4c2cnn4CCOCc2cccc(C4CC4)c2-c2ncc4ccc(n4n2)C3)n1]

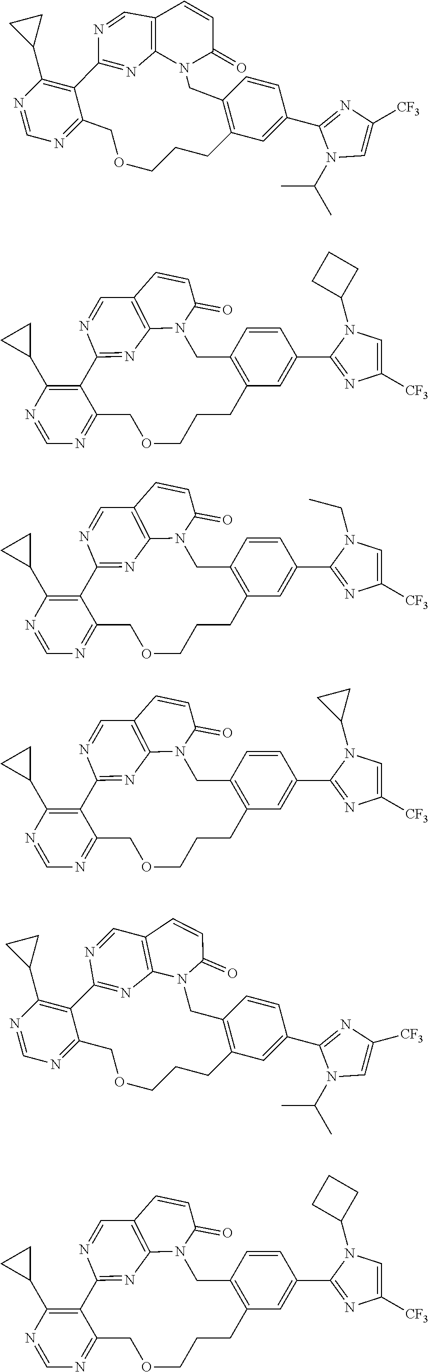

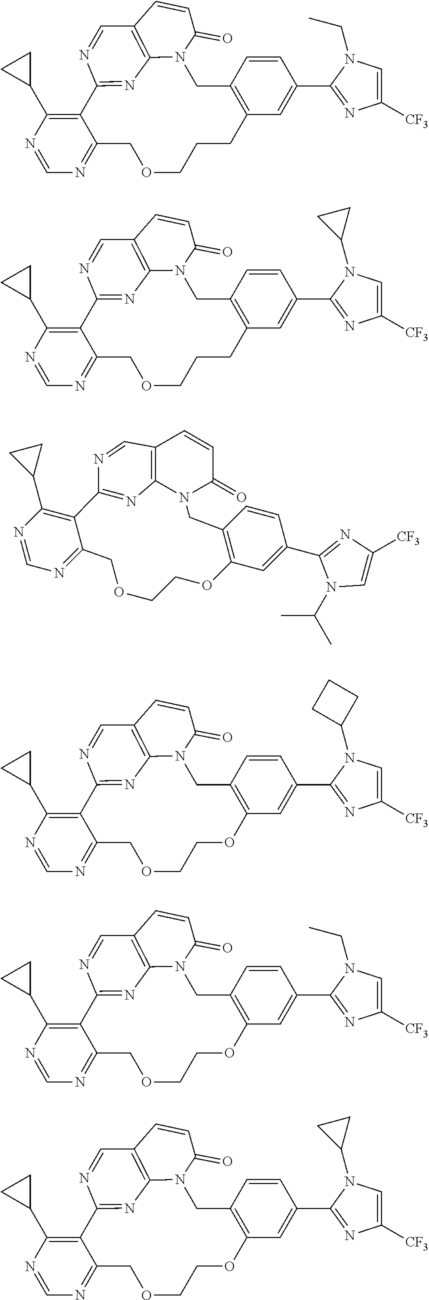

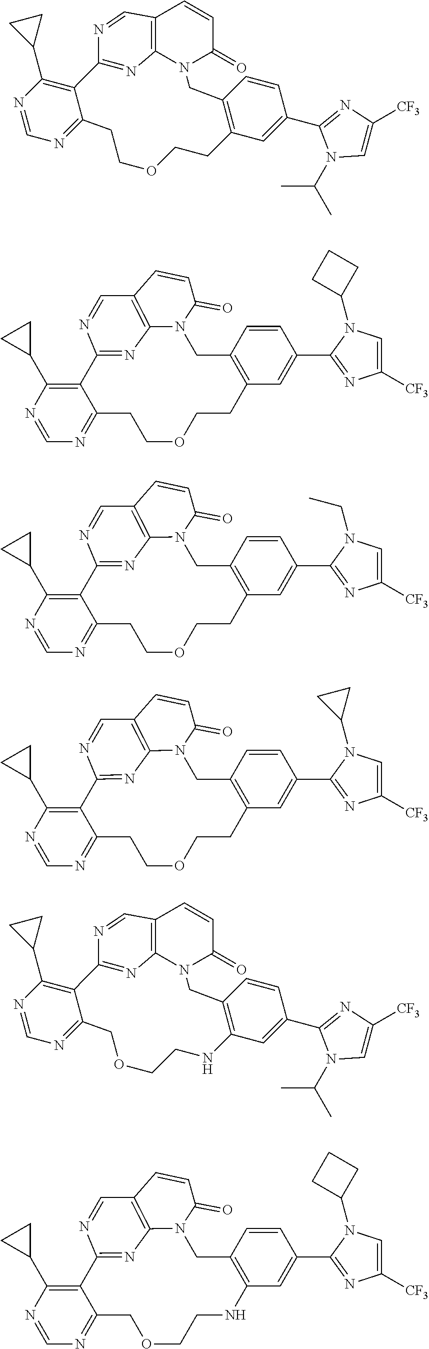

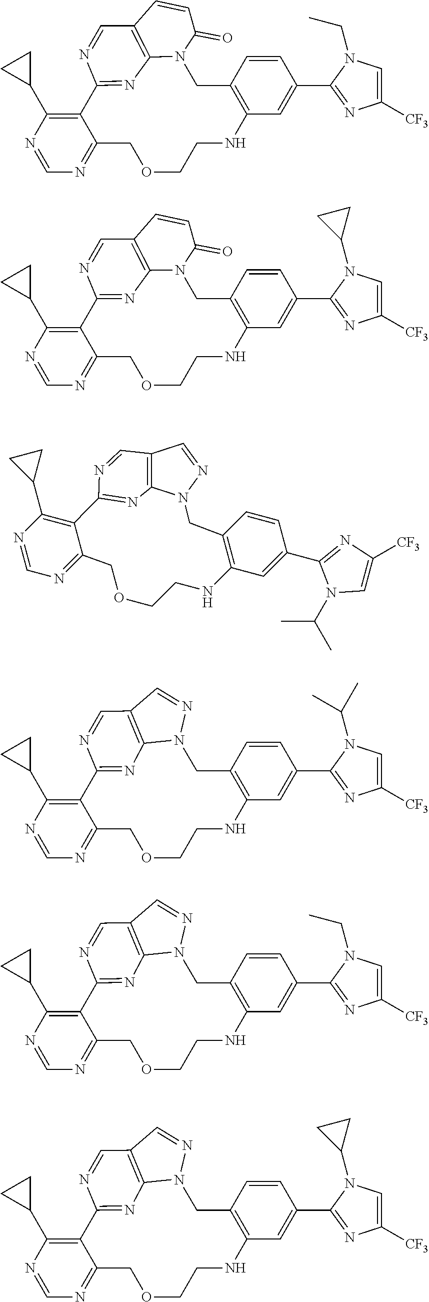

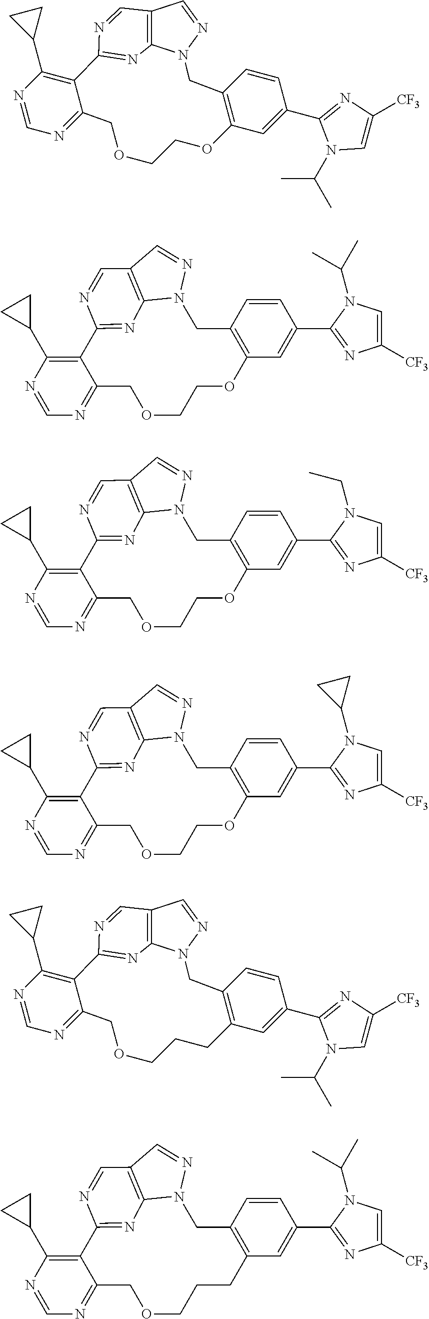

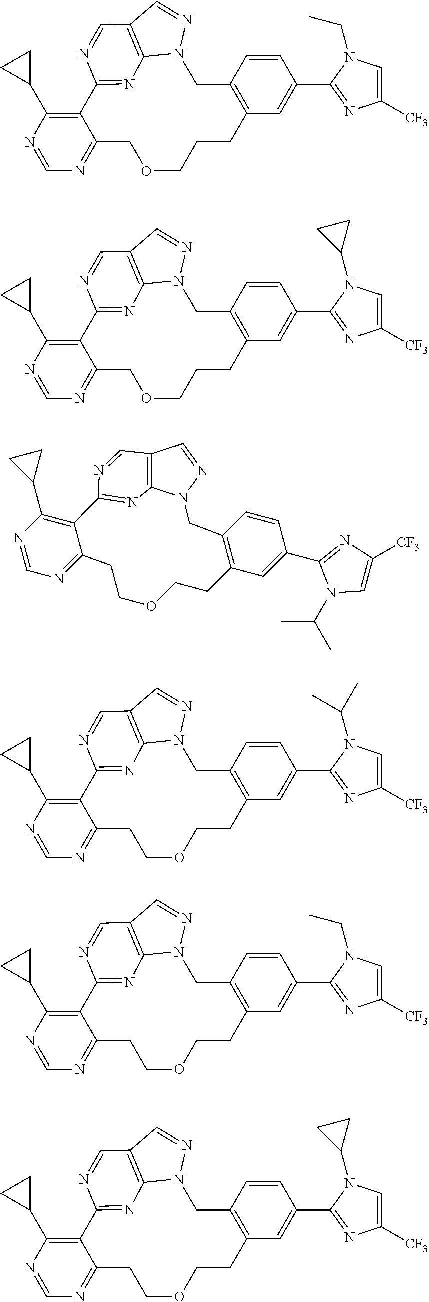

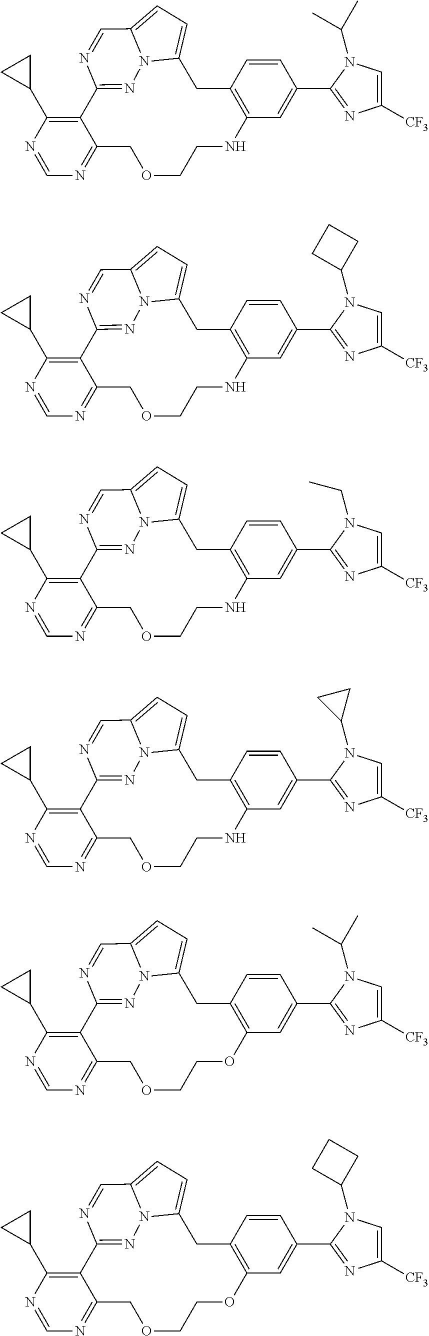 [SMILES: CC(C)n1cc(C(F)(F)F)nc1-c1ccc2c(c1)NCCOCc1ncnc(C3CC3)c1-c1ncc3ccc(n3n1)C2 ; FC(F)(F)c1cn(C2CCC2)c(-c2ccc3c(c2)NCCOCc2ncnc(C4CC4)c2-c2ncc4ccc(n4n2)C3)n1 ; CCn1cc(C(F)(F)F)nc1-c1ccc2c(c1)NCCOCc1ncnc(C3CC3)c1-c1ncc3ccc(n3n1)C2 ; FC(F)(F)c1cn(C2CC2)c(-c2ccc3c(c2)NCCOCc2ncnc(C4CC4)c2-c2ncc4ccc(n4n2)C3)n1 ; CC(C)n1cc(C(F)(F)F)nc1-c1ccc2c(c1)OCCOCc1ncnc(C3CC3)c1-c1ncc3ccc(n3n1)C2 ; FC(F)(F)c1cn(C2CCC2)c(-c2ccc3c(c2)OCCOCc2ncnc(C4CC4)c2-c2ncc4ccc(n4n2)C3)n1]

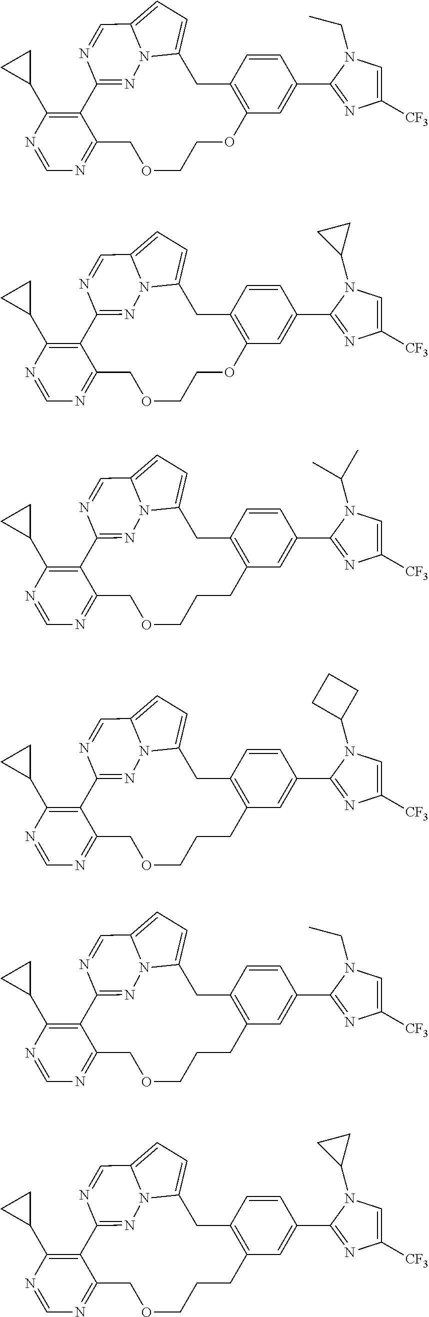 [SMILES: CCn1cc(C(F)(F)F)nc1-c1ccc2c(c1)OCCOCc1ncnc(C3CC3)c1-c1ncc3ccc(n3n1)C2 ; FC(F)(F)c1cn(C2CC2)c(-c2ccc3c(c2)OCCOCc2ncnc(C4CC4)c2-c2ncc4ccc(n4n2)C3)n1 ; CC(C)n1cc(C(F)(F)F)nc1-c1ccc2c(c1)CCCOCc1ncnc(C3CC3)c1-c1ncc3ccc(n3n1)C2 ; FC(F)(F)c1cn(C2CCC2)c(-c2ccc3c(c2)CCCOCc2ncnc(C4CC4)c2-c2ncc4ccc(n4n2)C3)n1 ; CCn1cc(C(F)(F)F)nc1-c1ccc2c(c1)CCCOCc1ncnc(C3CC3)c1-c1ncc3ccc(n3n1)C2 ; FC(F)(F)c1cn(C2CC2)c(-c2ccc3c(c2)CCCOCc2ncnc(C4CC4)c2-c2ncc4ccc(n4n2)C3)n1]

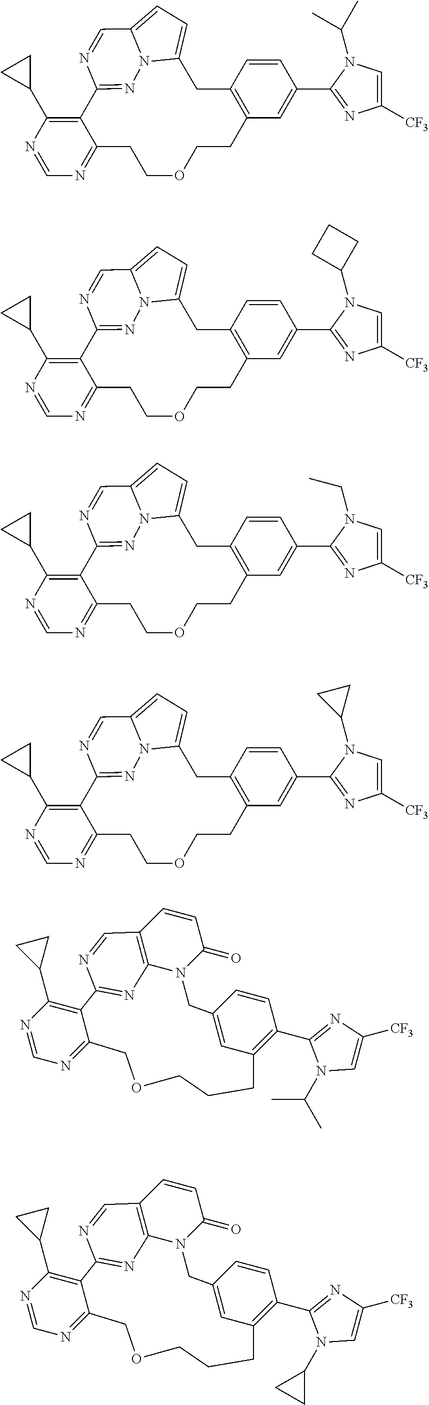 [SMILES: CC(C)n1cc(C(F)(F)F)nc1-c1ccc2c(c1)CCOCCc1ncnc(C3CC3)c1-c1ncc3ccc(n3n1)C2 ; FC(F)(F)c1cn(C2CCC2)c(-c2ccc3c(c2)CCOCCc2ncnc(C4CC4)c2-c2ncc4ccc(n4n2)C3)n1 ; CCn1cc(C(F)(F)F)nc1-c1ccc2c(c1)CCOCCc1ncnc(C3CC3)c1-c1ncc3ccc(n3n1)C2 ; FC(F)(F)c1cn(C2CC2)c(-c2ccc3c(c2)CCOCCc2ncnc(C4CC4)c2-c2ncc4ccc(n4n2)C3)n1 ; CC(C)n1cc(C(F)(F)F)nc1-c1ccc2cc1CCCOCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)c(c1)CCCOCc1ncnc(C2CC2)c1-3]

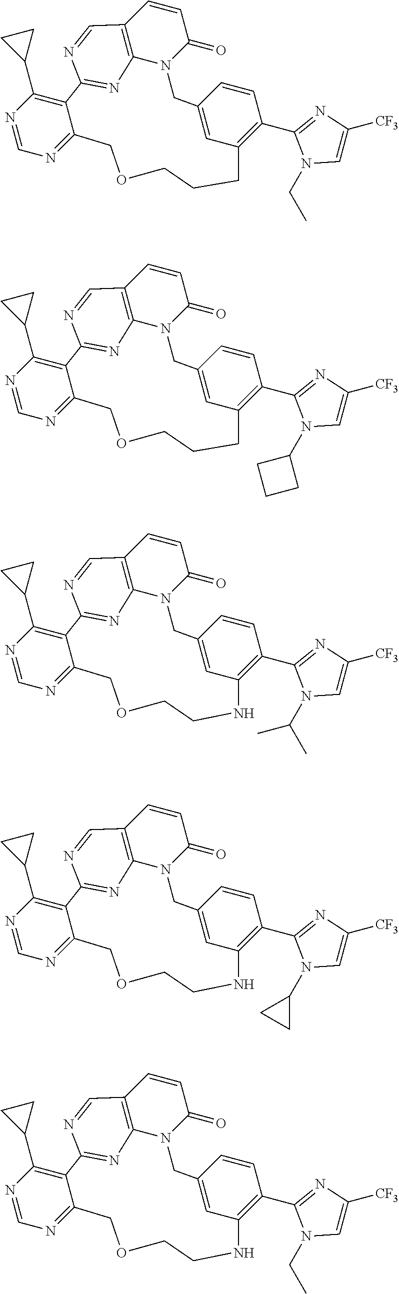 [SMILES: CCn1cc(C(F)(F)F)nc1-c1ccc2cc1CCCOCc1ncnc(C3CC3)c1-c1ncc3ccc(=O)n(c3n1)C2 ; O=c1ccc2cnc3nc2n1Cc1ccc(-c2nc(C(F)(F)F)cn2C2CCC2)c(c1)CCCOCc1ncnc(C2CC2)c1-3 ; CC(C)n1cc(C(F)(F)F)nc1-c1ccc2cc1NCCOCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)c(c1)NCCOCc1ncnc(C2CC2)c1-3 ; CCn1cc(C(F)(F)F)nc1-c1ccc2cc1NCCOCc1ncnc(C3CC3)c1-c1ncc3ccc(=O)n(c3n1)C2]

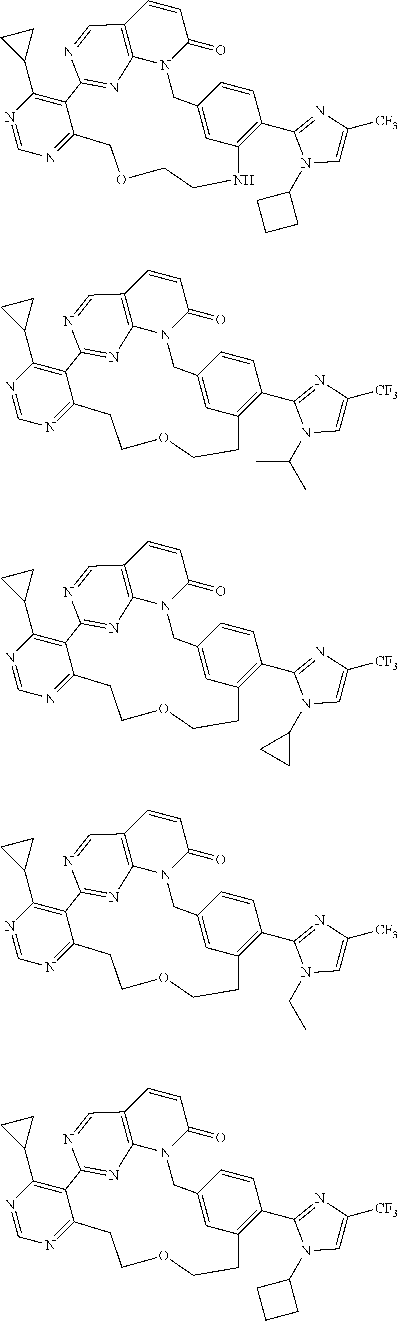 [SMILES: O=c1ccc2cnc3nc2n1Cc1ccc(-c2nc(C(F)(F)F)cn2C2CCC2)c(c1)NCCOCc1ncnc(C2CC2)c1-3 ; CC(C)n1cc(C(F)(F)F)nc1-c1ccc2cc1CCOCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)c(c1)CCOCCc1ncnc(C2CC2)c1-3 ; CCn1cc(C(F)(F)F)nc1-c1ccc2cc1CCOCCc1ncnc(C3CC3)c1-c1ncc3ccc(=O)n(c3n1)C2 ; O=c1ccc2cnc3nc2n1Cc1ccc(-c2nc(C(F)(F)F)cn2C2CCC2)c(c1)CCOCCc1ncnc(C2CC2)c1-3]

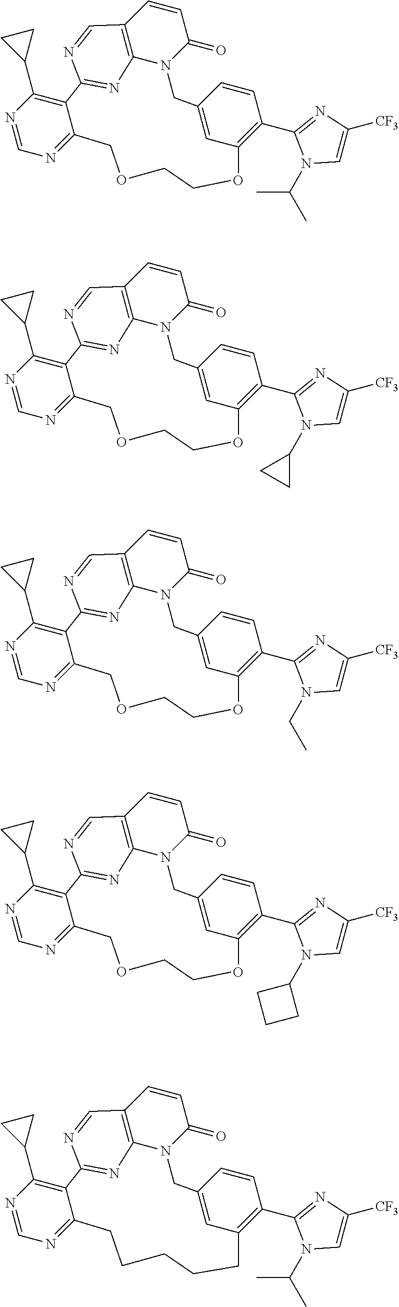 [SMILES: CC(C)n1cc(C(F)(F)F)nc1-c1ccc2cc1OCCOCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)c(c1)OCCOCc1ncnc(C2CC2)c1-3 ; CCn1cc(C(F)(F)F)nc1-c1ccc2cc1OCCOCc1ncnc(C3CC3)c1-c1ncc3ccc(=O)n(c3n1)C2 ; O=c1ccc2cnc3nc2n1Cc1ccc(-c2nc(C(F)(F)F)cn2C2CCC2)c(c1)OCCOCc1ncnc(C2CC2)c1-3 ; CC(C)n1cc(C(F)(F)F)nc1-c1ccc2cc1CCCCCc1ncnc(C3CC3)c1-c1ncc3ccc(=O)n(c3n1)C2]

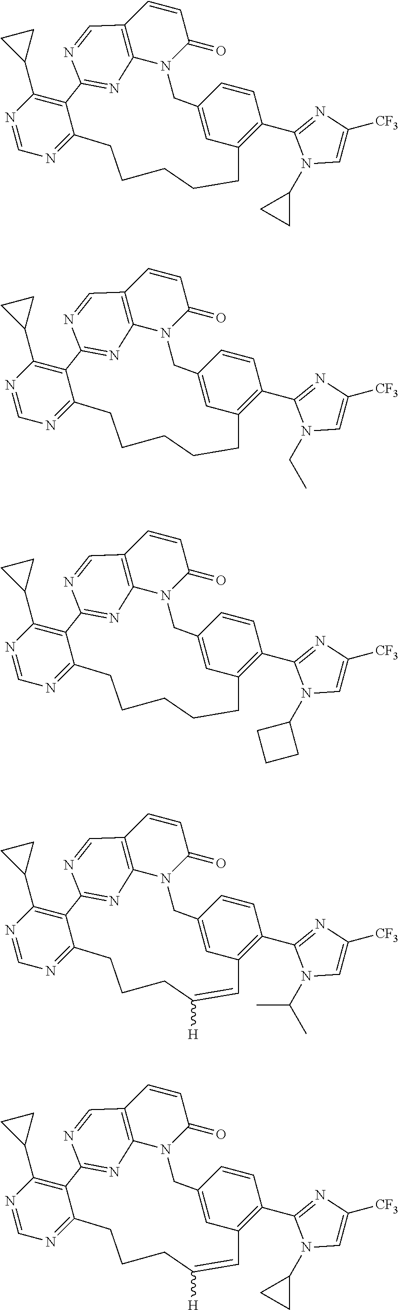 [SMILES: O=c1ccc2cnc3nc2n1Cc1ccc(-c2nc(C(F)(F)F)cn2C2CC2)c(c1)CCCCCc1ncnc(C2CC2)c1-3 ; CCn1cc(C(F)(F)F)nc1-c1ccc2cc1CCCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CCC2)c(c1)CCCCCc1ncnc(C2CC2)c1-3 ; CC(C)n1cc(C(F)(F)F)nc1-c1ccc2cc1C=CCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CC2)c(c1)C=CCCCc1ncnc(C2CC2)c1-3]

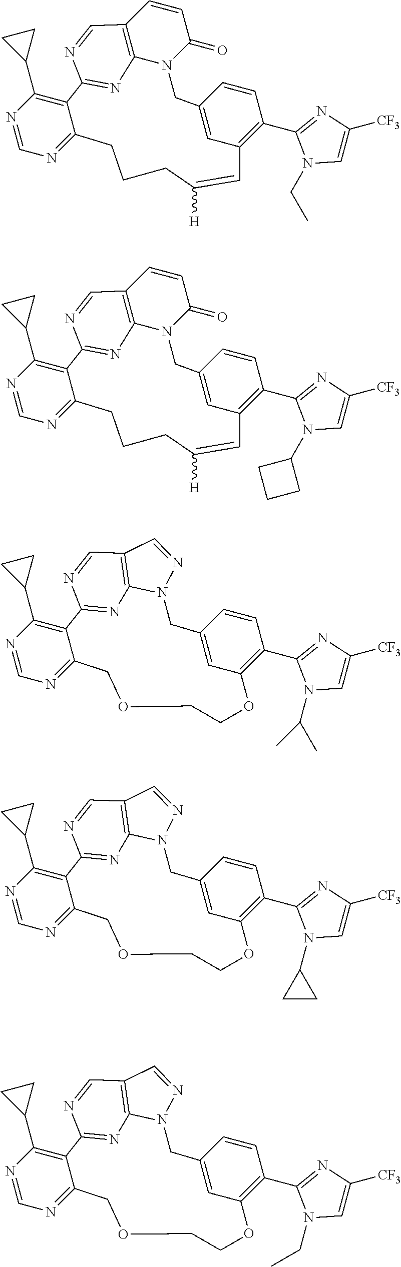 [SMILES: CCn1cc(C(F)(F)F)nc1-c1ccc2cc1C=CCCCc1ncnc(C3CC3)c1-c1ncc3ccc(=O)n(c3n1)C2 ; O=c1ccc2cnc3nc2n1Cc1ccc(-c2nc(C(F)(F)F)cn2C2CCC2)c(c1)C=CCCCc1ncnc(C2CC2)c1-3 ; CC(C)n1cc(C(F)(F)F)nc1-c1ccc2cc1OCCOCc1ncnc(C3CC3)c1-c1ncc3cnn(c3n1)C2 ; FC(F)(F)c1cn(C2CC2)c(-c2ccc3cc2OCCOCc2ncnc(C4CC4)c2-c2ncc4cnn(c4n2)C3)n1 ; CCn1cc(C(F)(F)F)nc1-c1ccc2cc1OCCOCc1ncnc(C3CC3)c1-c1ncc3cnn(c3n1)C2]

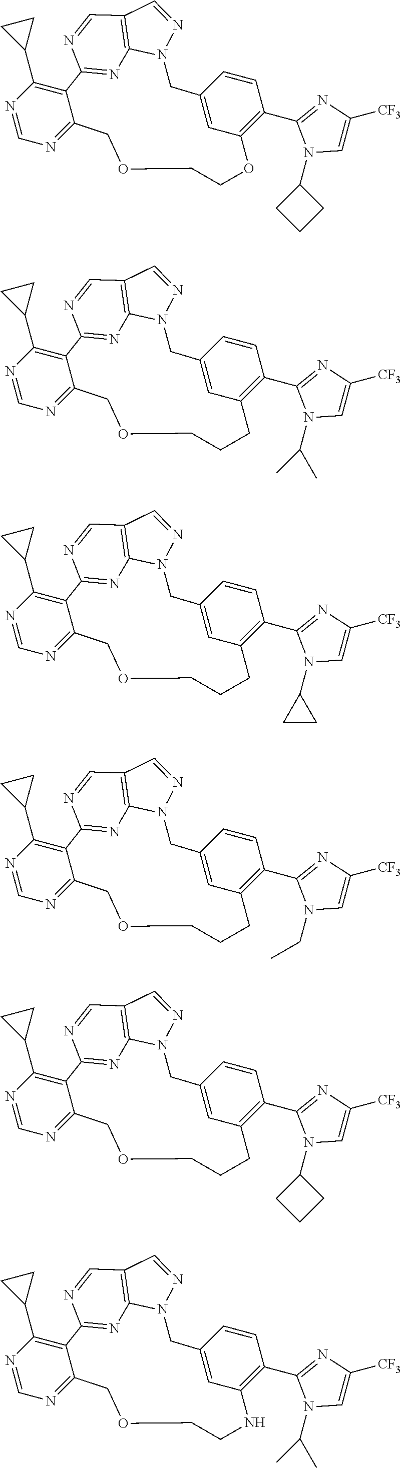 [SMILES: FC(F)(F)c1cn(C2CCC2)c(-c2ccc3cc2OCCOCc2ncnc(C4CC4)c2-c2ncc4cnn(c4n2)C3)n1 ; CC(C)n1cc(C(F)(F)F)nc1-c1ccc2cc1CCCOCc1ncnc(C3CC3)c1-c1ncc3cnn(c3n1)C2 ; FC(F)(F)c1cn(C2CC2)c(-c2ccc3cc2CCCOCc2ncnc(C4CC4)c2-c2ncc4cnn(c4n2)C3)n1 ; CCn1cc(C(F)(F)F)nc1-c1ccc2cc1CCCOCc1ncnc(C3CC3)c1-c1ncc3cnn(c3n1)C2 ; FC(F)(F)c1cn(C2CCC2)c(-c2ccc3cc2CCCOCc2ncnc(C4CC4)c2-c2ncc4cnn(c4n2)C3)n1 ; CC(C)n1cc(C(F)(F)F)nc1-c1ccc2cc1NCCOCc1ncnc(C3CC3)c1-c1ncc3cnn(c3n1)C2]

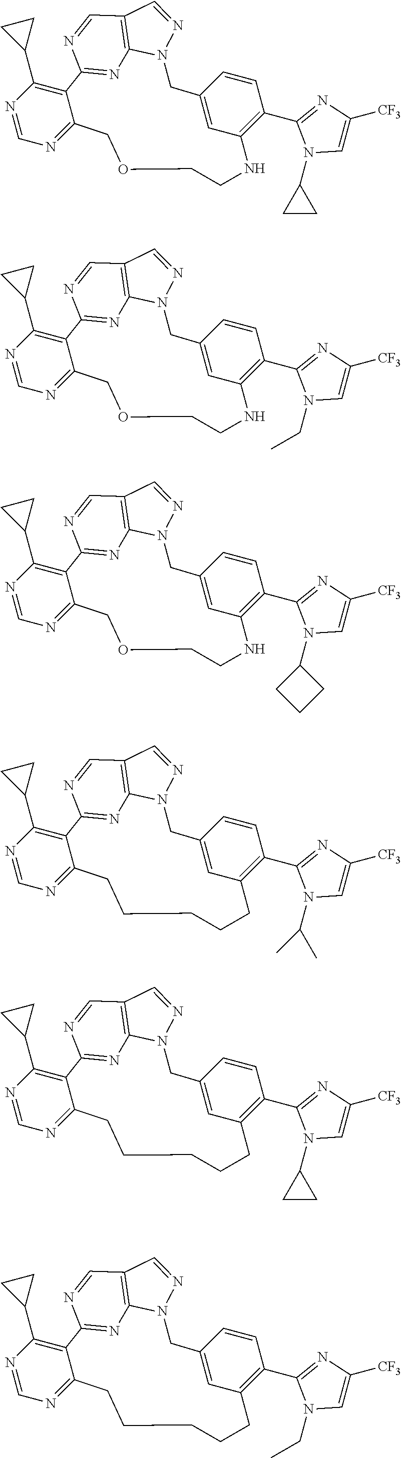 [SMILES: FC(F)(F)c1cn(C2CC2)c(-c2ccc3cc2NCCOCc2ncnc(C4CC4)c2-c2ncc4cnn(c4n2)C3)n1 ; CCn1cc(C(F)(F)F)nc1-c1ccc2cc1NCCOCc1ncnc(C3CC3)c1-c1ncc3cnn(c3n1)C2 ; FC(F)(F)c1cn(C2CCC2)c(-c2ccc3cc2NCCOCc2ncnc(C4CC4)c2-c2ncc4cnn(c4n2)C3)n1 ; CC(C)n1cc(C(F)(F)F)nc1-c1ccc2cc1CCCCCc1ncnc(C3CC3)c1-c1ncc3cnn(c3n1)C2 ; FC(F)(F)c1cn(C2CC2)c(-c2ccc3cc2CCCCCc2ncnc(C4CC4)c2-c2ncc4cnn(c4n2)C3)n1 ; CCn1cc(C(F)(F)F)nc1-c1ccc2cc1CCCCCc1ncnc(C3CC3)c1-c1ncc3cnn(c3n1)C2]

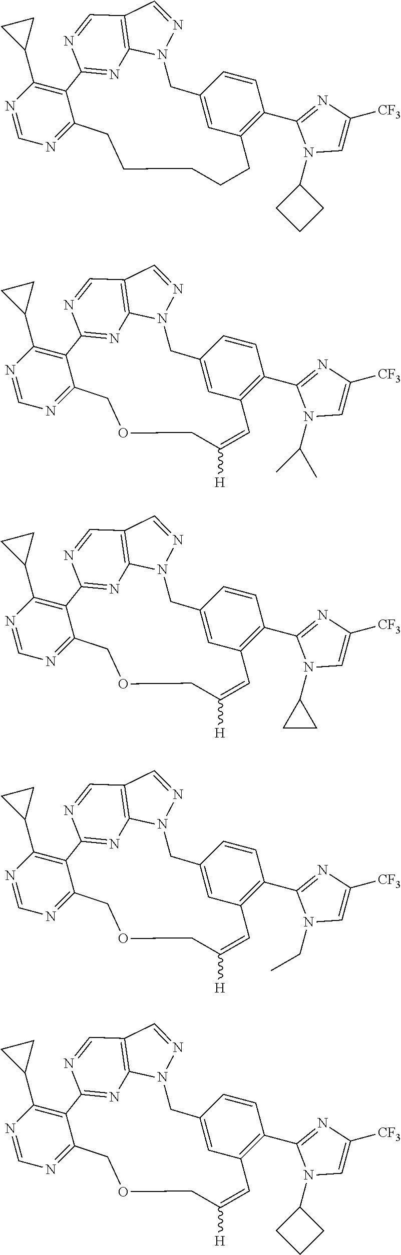 [SMILES: FC(F)(F)c1cn(C2CCC2)c(-c2ccc3cc2CCCCCc2ncnc(C4CC4)c2-c2ncc4cnn(c4n2)C3)n1 ; CC(C)n1cc(C(F)(F)F)nc1-c1ccc2cc1C=CCOCc1ncnc(C3CC3)c1-c1ncc3cnn(c3n1)C2 ; FC(F)(F)c1cn(C2CC2)c(-c2ccc3cc2C=CCOCc2ncnc(C4CC4)c2-c2ncc4cnn(c4n2)C3)n1 ; CCn1cc(C(F)(F)F)nc1-c1ccc2cc1C=CCOCc1ncnc(C3CC3)c1-c1ncc3cnn(c3n1)C2 ; FC(F)(F)c1cn(C2CCC2)c(-c2ccc3cc2C=CCOCc2ncnc(C4CC4)c2-c2ncc4cnn(c4n2)C3)n1]

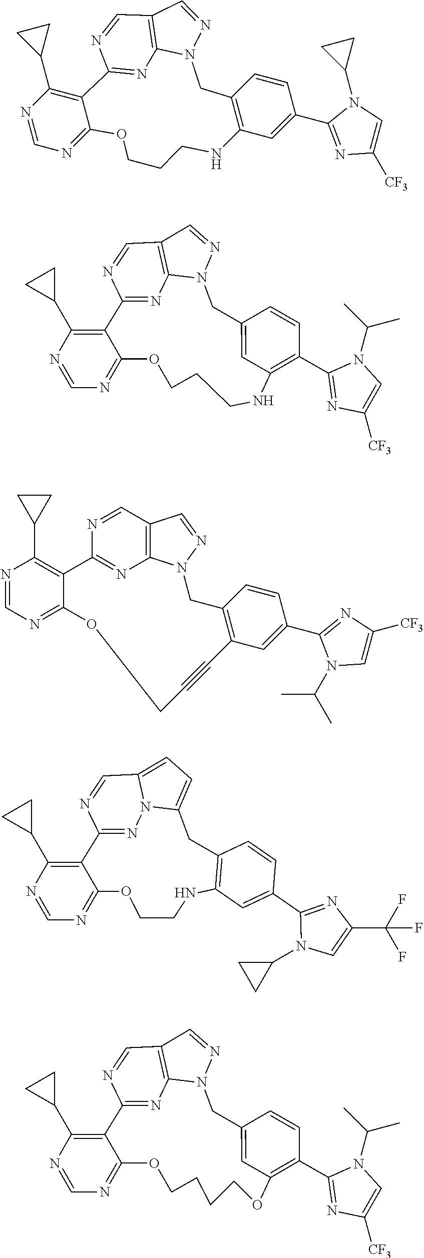

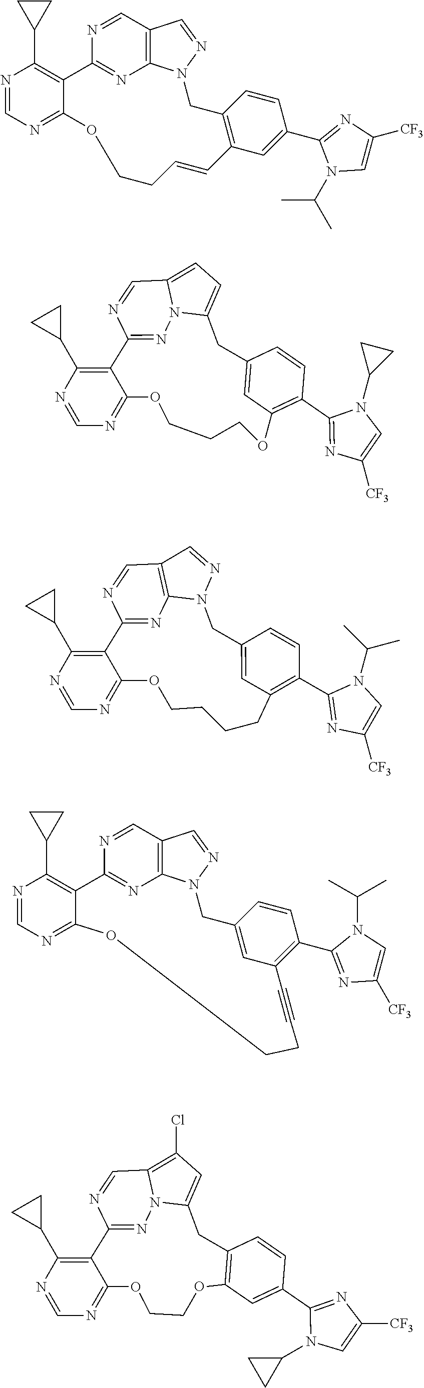 [SMILES: CC(C)n1cc(C(F)(F)F)nc1-c1ccc2c(c1)/C=C/CCOc1ncnc(C3CC3)c1-c1ncc3cnn(c3n1)C2 ; FC(F)(F)c1cn(C2CC2)c(-c2ccc3cc2OCCCOc2ncnc(C4CC4)c2-c2ncc4ccc(n4n2)C3)n1 ; CC(C)n1cc(C(F)(F)F)nc1-c1ccc2cc1CCCCOc1ncnc(C3CC3)c1-c1ncc3cnn(c3n1)C2 ; CC(C)n1cc(C(F)(F)F)nc1-c1ccc2cc1C#CCCOc1ncnc(C3CC3)c1-c1ncc3cnn(c3n1)C2 ; FC(F)(F)c1cn(C2CC2)c(-c2ccc3c(c2)OCCOc2ncnc(C4CC4)c2-c2ncc4c(Cl)cc(n4n2)C3)n1]

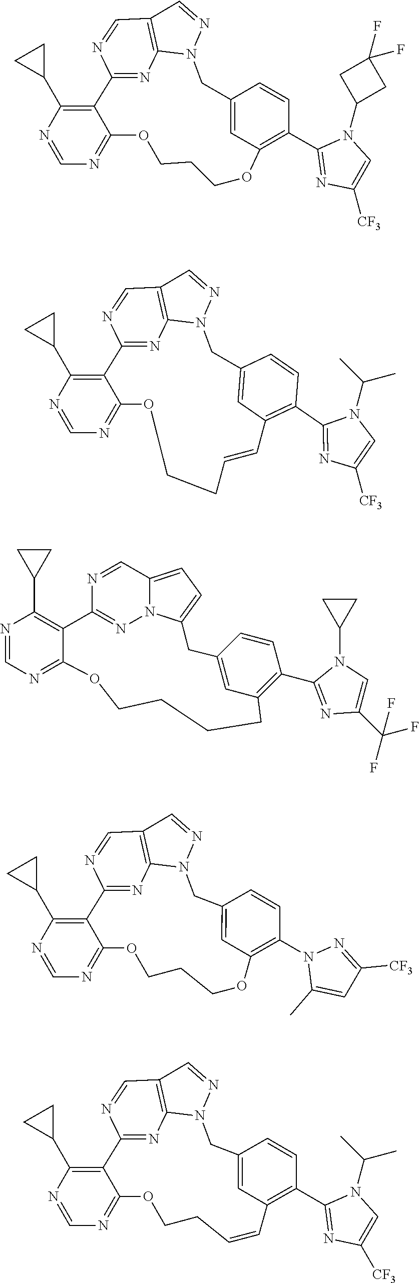 [SMILES: FC1(F)CC(n2cc(C(F)(F)F)nc2-c2ccc3cc2OCCCOc2ncnc(C4CC4)c2-c2ncc4cnn(c4n2)C3)C1 ; CC(C)n1cc(C(F)(F)F)nc1-c1ccc2cc1/C=C/CCOc1ncnc(C3CC3)c1-c1ncc3cnn(c3n1)C2 ; FC(F)(F)c1cn(C2CC2)c(-c2ccc3cc2CCCCOc2ncnc(C4CC4)c2-c2ncc4ccc(n4n2)C3)n1 ; Cc1cc(C(F)(F)F)nn1-c1ccc2cc1OCCCOc1ncnc(C3CC3)c1-c1ncc3cnn(c3n1)C2 ; CC(C)n1cc(C(F)(F)F)nc1-c1ccc2cc1/C=C\CCOc1ncnc(C3CC3)c1-c1ncc3cnn(c3n1)C2]

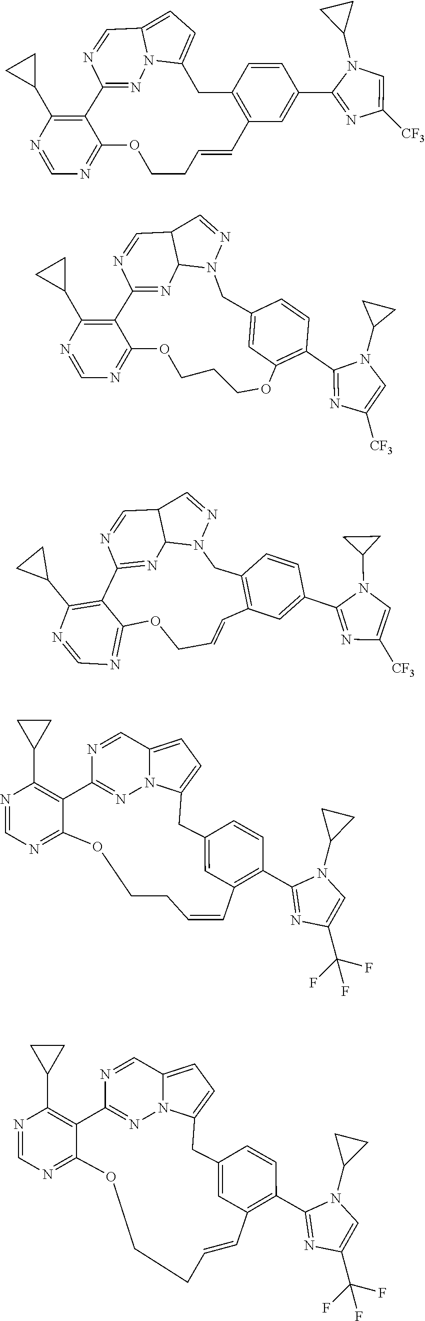

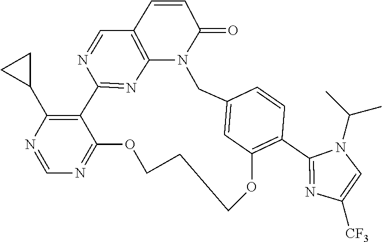 [SMILES: CC(C)n1cc(C(F)(F)F)nc1-c1ccc2cc1OCCCOc1ncnc(C3CC3)c1-c1ncc3ccc(=O)n(c3n1)C2]

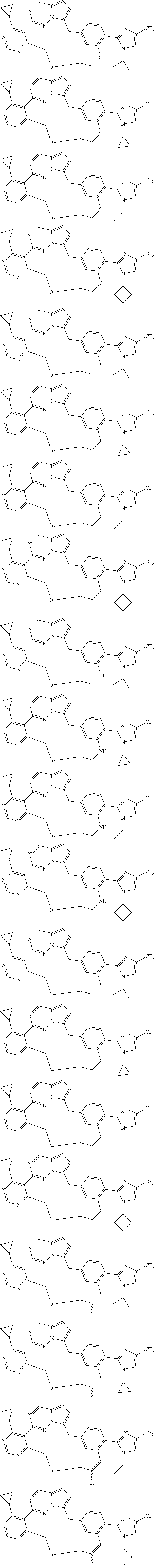

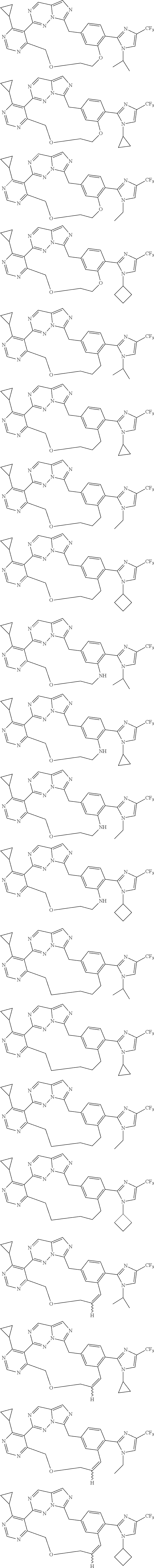

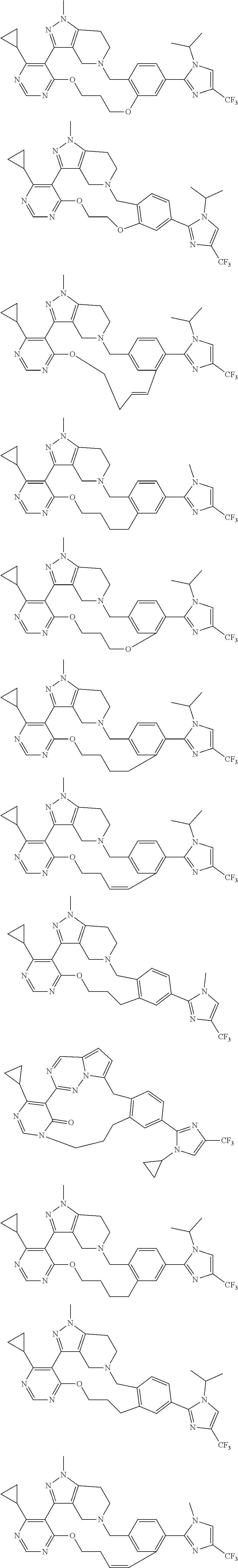

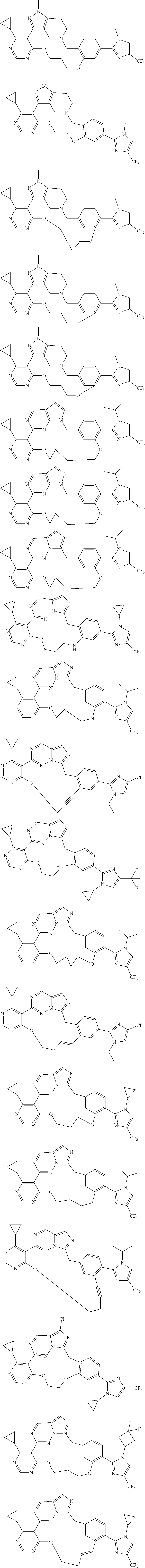

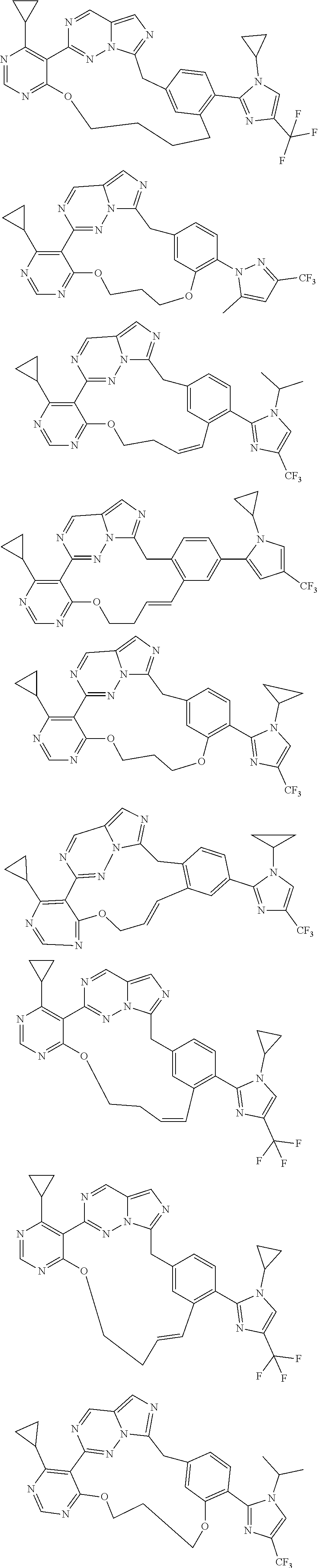

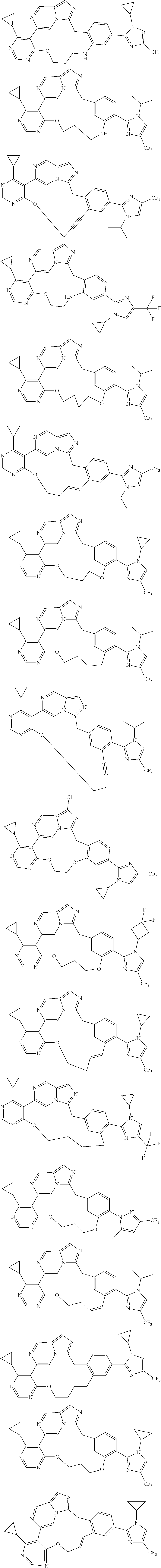

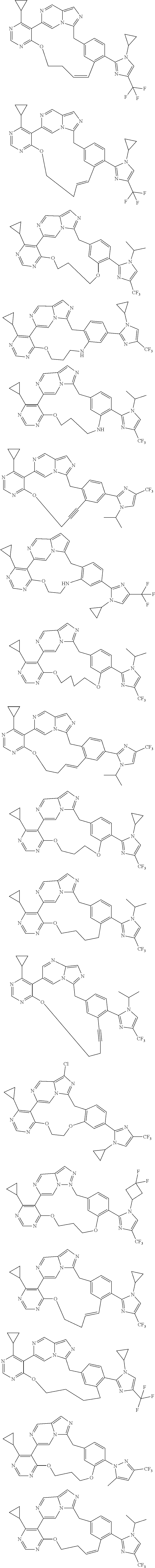

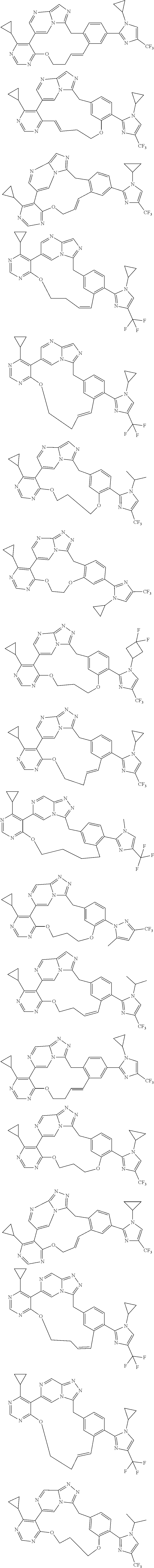

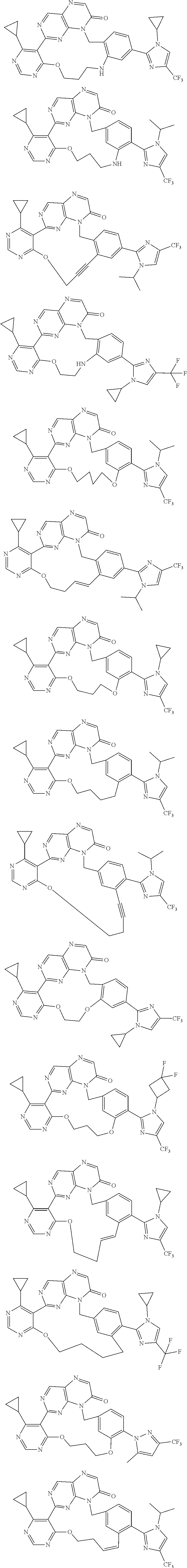

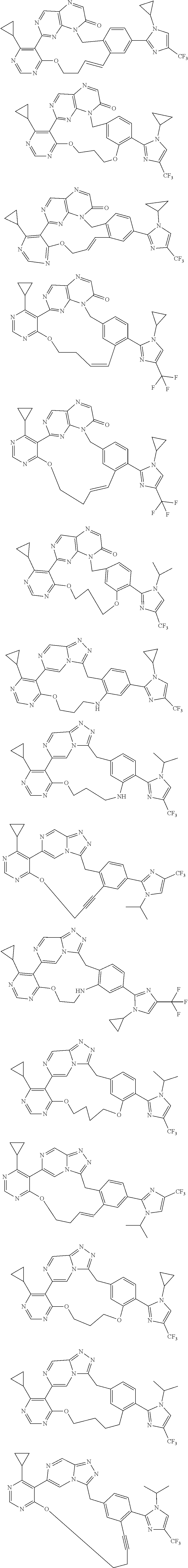

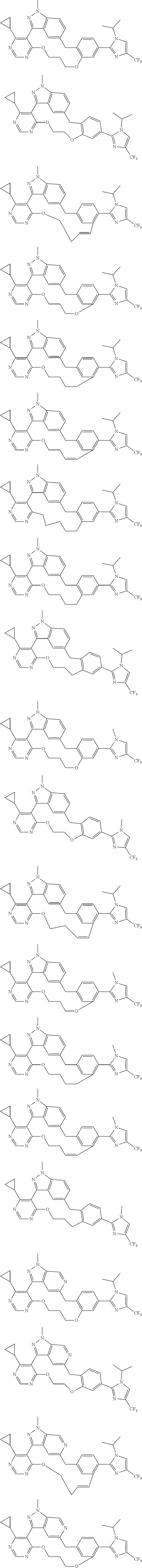

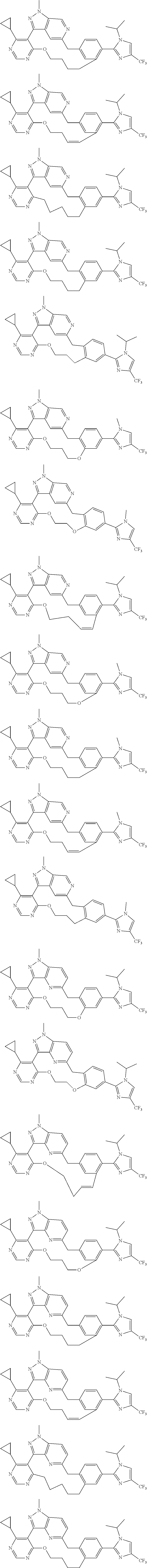

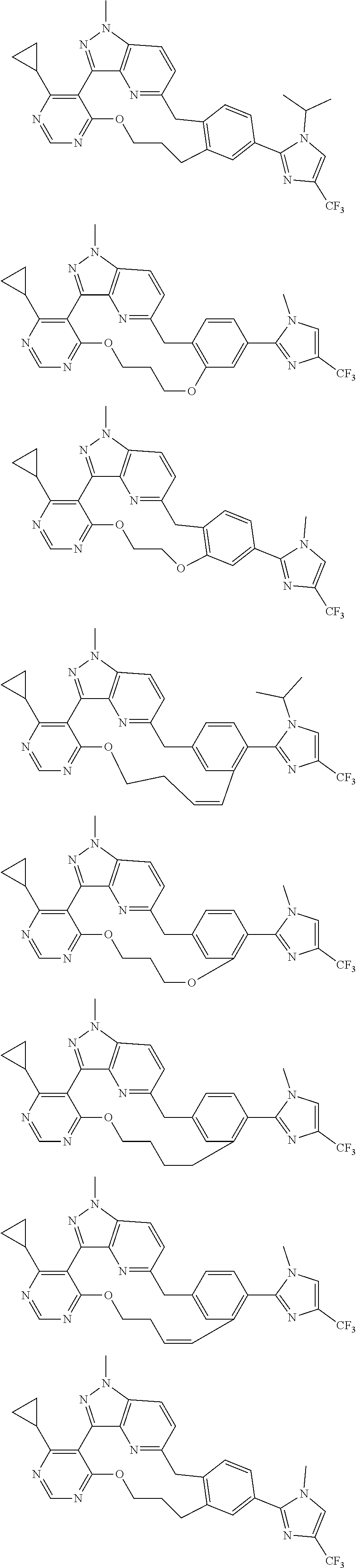

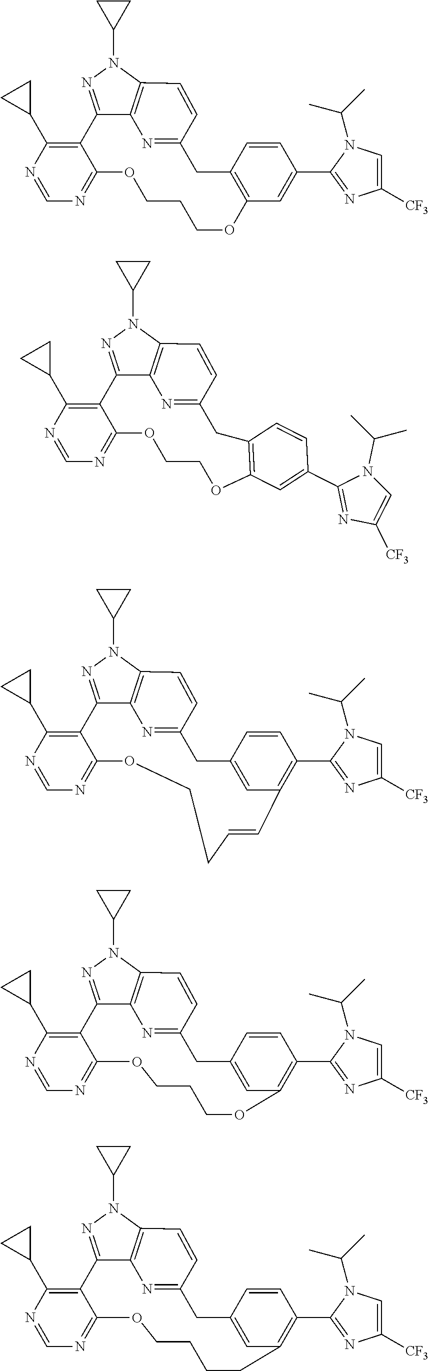 [SMILES: CC(C)n1cc(C(F)(F)F)nc1-c1ccc2c(c1)OCCCOc1ncnc(C3CC3)c1-c1nn(C3CC3)c3ccc(nc13)C2 ; CC(C)n1cc(C(F)(F)F)nc1-c1ccc2c(c1)OCCOc1ncnc(C3CC3)c1-c1nn(C3CC3)c3ccc(nc13)C2 ; CC(C)n1cc(C(F)(F)F)nc1-c1ccc2cc1/C=C/CCOc1ncnc(C3CC3)c1-c1nn(C3CC3)c3ccc(nc13)C2 ; CC(C)n1cc(C(F)(F)F)nc1-c1ccc2cc1OCCCOc1ncnc(C3CC3)c1-c1nn(C3CC3)c3ccc(nc13)C2 ; CC(C)n1cc(C(F)(F)F)nc1-c1ccc2cc1CCCCOc1ncnc(C3CC3)c1-c1nn(C3CC3)c3ccc(nc13)C2]

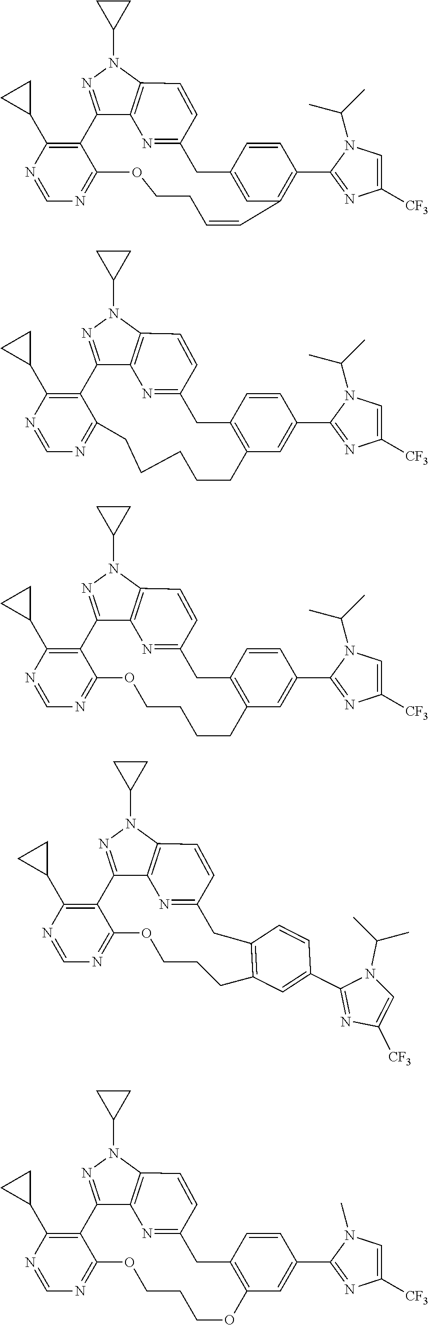

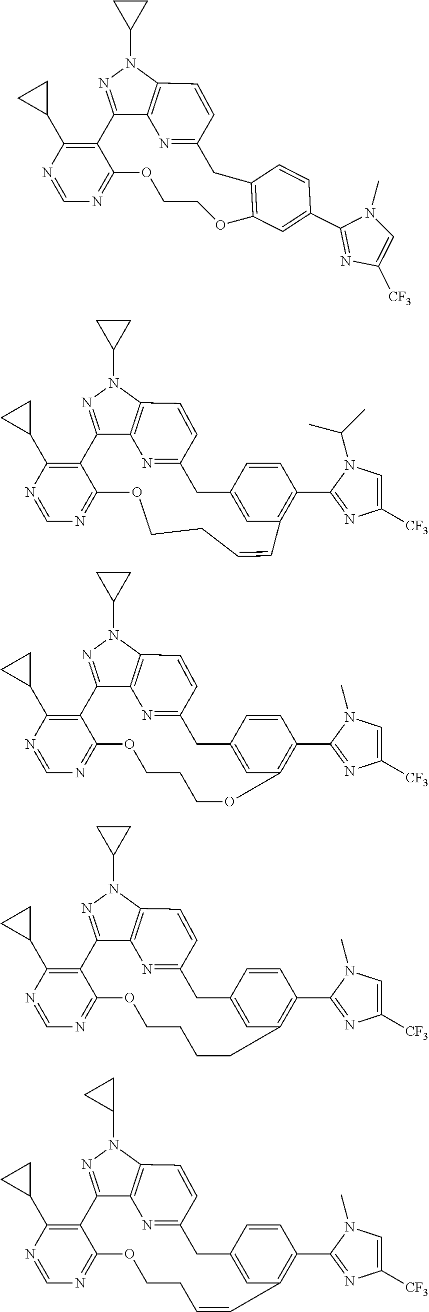

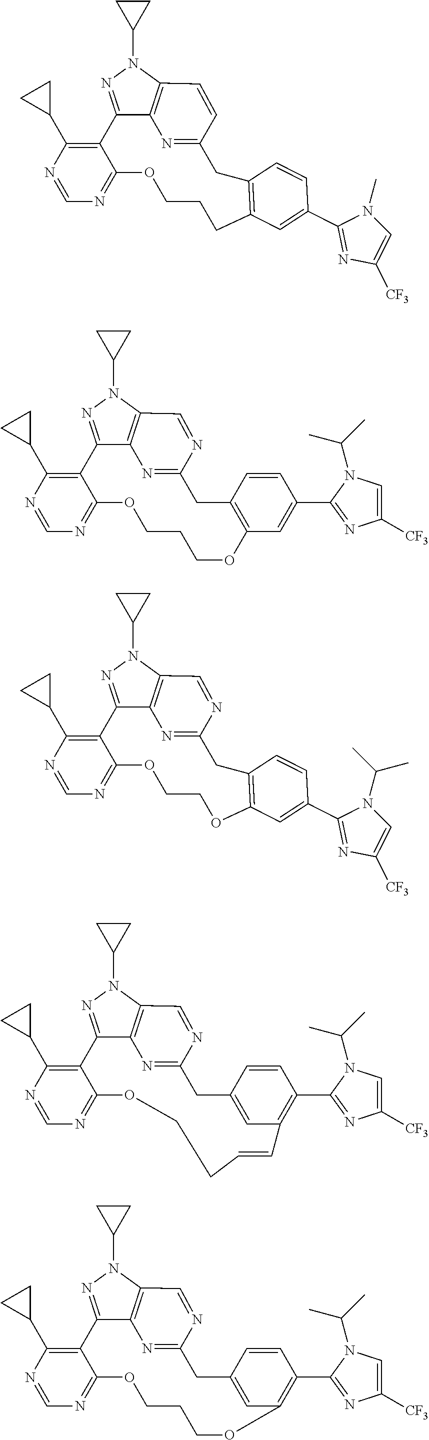 [SMILES: Cn1cc(C(F)(F)F)nc1-c1ccc2c(c1)CCCOc1ncnc(C3CC3)c1-c1nn(C3CC3)c3ccc(nc13)C2 ; CC(C)n1cc(C(F)(F)F)nc1-c1ccc2c(c1)OCCCOc1ncnc(C3CC3)c1-c1nn(C3CC3)c3cnc(nc13)C2 ; CC(C)n1cc(C(F)(F)F)nc1-c1ccc2c(c1)OCCOc1ncnc(C3CC3)c1-c1nn(C3CC3)c3cnc(nc13)C2 ; CC(C)n1cc(C(F)(F)F)nc1-c1ccc2cc1/C=C/CCOc1ncnc(C3CC3)c1-c1nn(C3CC3)c3cnc(nc13)C2 ; CC(C)n1cc(C(F)(F)F)nc1-c1ccc2cc1OCCCOc1ncnc(C3CC3)c1-c1nn(C3CC3)c3cnc(nc13)C2]

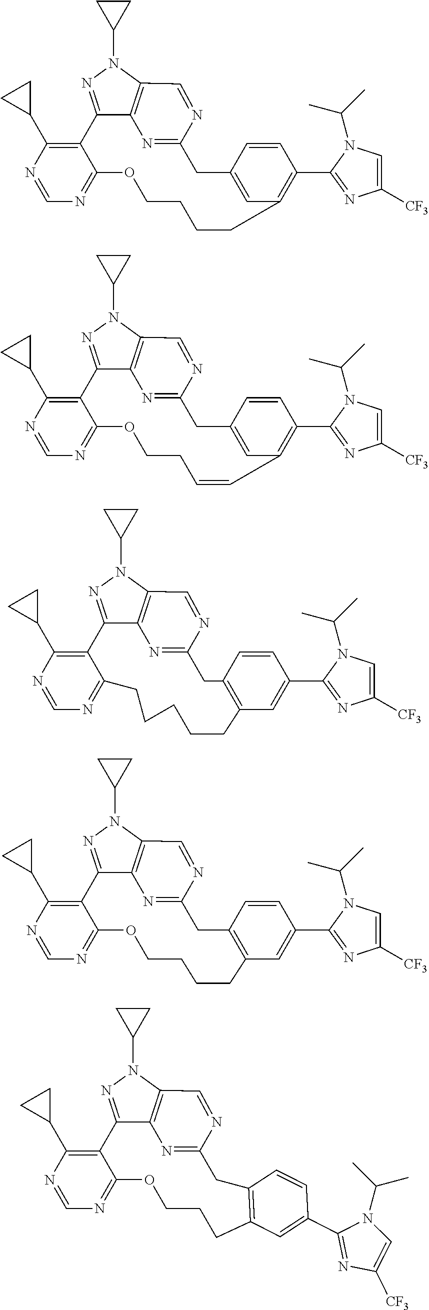

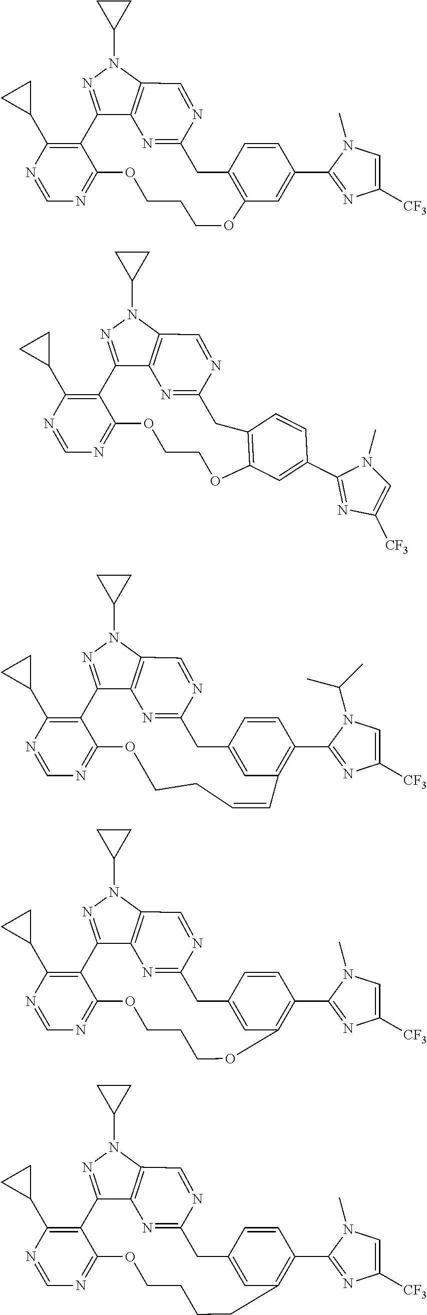 [SMILES: Cn1cc(C(F)(F)F)nc1-c1ccc2c(c1)OCCCOc1ncnc(C3CC3)c1-c1nn(C3CC3)c3cnc(nc13)C2 ; Cn1cc(C(F)(F)F)nc1-c1ccc2c(c1)OCCOc1ncnc(C3CC3)c1-c1nn(C3CC3)c3cnc(nc13)C2 ; CC(C)n1cc(C(F)(F)F)nc1-c1ccc2cc1/C=C\CCOc1ncnc(C3CC3)c1-c1nn(C3CC3)c3cnc(nc13)C2 ; Cn1cc(C(F)(F)F)nc1-c1ccc2cc1OCCCOc1ncnc(C3CC3)c1-c1nn(C3CC3)c3cnc(nc13)C2 ; Cn1cc(C(F)(F)F)nc1-c1ccc2cc1CCCCOc1ncnc(C3CC3)c1-c1nn(C3CC3)c3cnc(nc13)C2]

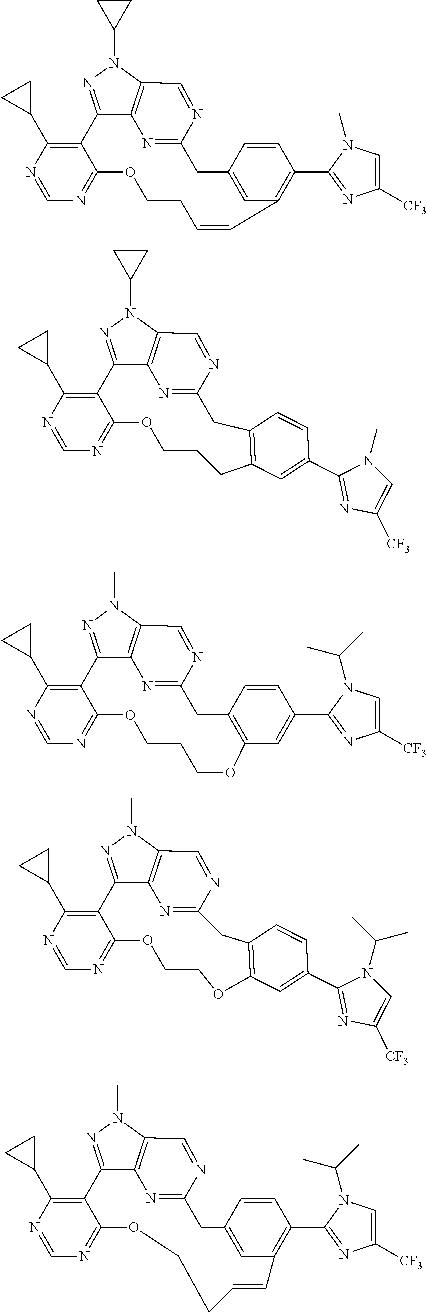 [SMILES: Cn1cc(C(F)(F)F)nc1-c1ccc2cc1/C=C\CCOc1ncnc(C3CC3)c1-c1nn(C3CC3)c3cnc(nc13)C2 ; Cn1cc(C(F)(F)F)nc1-c1ccc2c(c1)CCCOc1ncnc(C3CC3)c1-c1nn(C3CC3)c3cnc(nc13)C2 ; CC(C)n1cc(C(F)(F)F)nc1-c1ccc2c(c1)OCCCOc1ncnc(C3CC3)c1-c1nn(C)c3cnc(nc13)C2 ; CC(C)n1cc(C(F)(F)F)nc1-c1ccc2c(c1)OCCOc1ncnc(C3CC3)c1-c1nn(C)c3cnc(nc13)C2 ; CC(C)n1cc(C(F)(F)F)nc1-c1ccc2cc1/C=C/CCOc1ncnc(C3CC3)c1-c1nn(C)c3cnc(nc13)C2]

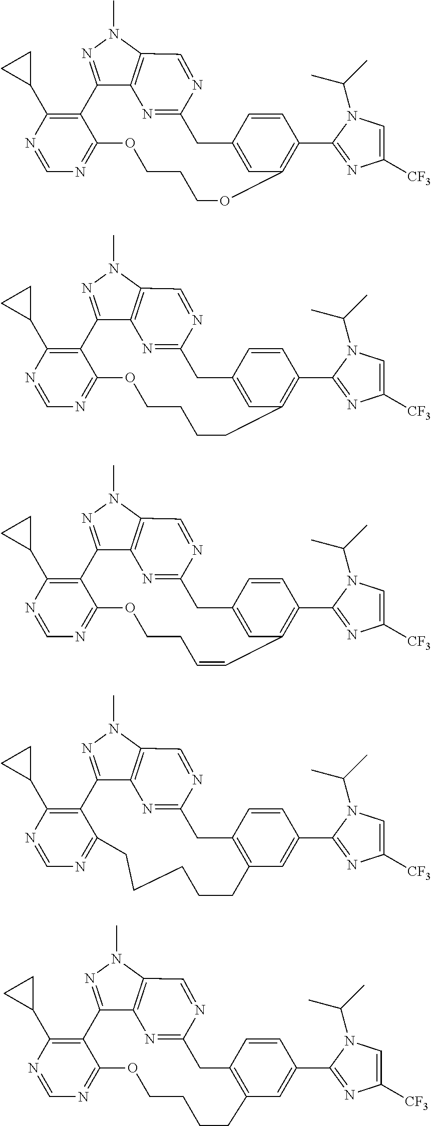

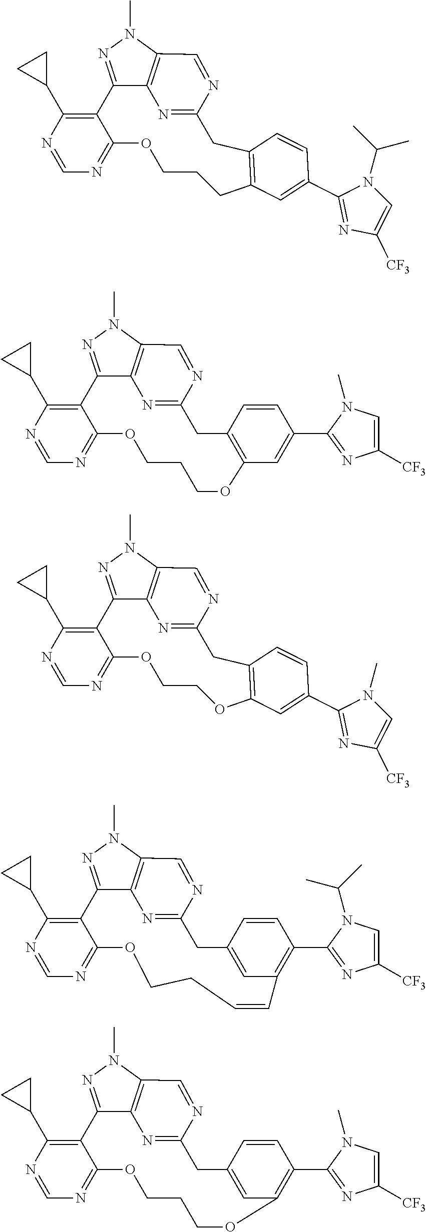 [SMILES: CC(C)n1cc(C(F)(F)F)nc1-c1ccc2c(c1)CCCOc1ncnc(C3CC3)c1-c1nn(C)c3cnc(nc13)C2 ; Cn1cc(C(F)(F)F)nc1-c1ccc2c(c1)OCCCOc1ncnc(C3CC3)c1-c1nn(C)c3cnc(nc13)C2 ; Cn1cc(C(F)(F)F)nc1-c1ccc2c(c1)OCCOc1ncnc(C3CC3)c1-c1nn(C)c3cnc(nc13)C2 ; CC(C)n1cc(C(F)(F)F)nc1-c1ccc2cc1/C=C\CCOc1ncnc(C3CC3)c1-c1nn(C)c3cnc(nc13)C2 ; Cn1cc(C(F)(F)F)nc1-c1ccc2cc1OCCCOc1ncnc(C3CC3)c1-c1nn(C)c3cnc(nc13)C2]

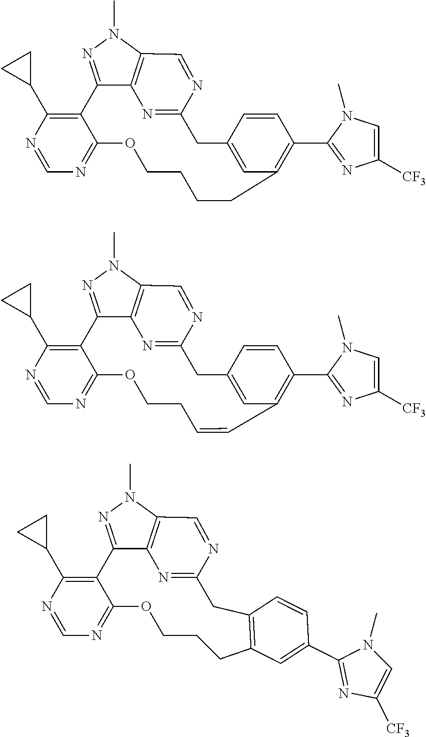 [SMILES: Cn1cc(C(F)(F)F)nc1-c1ccc2cc1CCCCOc1ncnc(C3CC3)c1-c1nn(C)c3cnc(nc13)C2 ; Cn1cc(C(F)(F)F)nc1-c1ccc2cc1/C=C\CCOc1ncnc(C3CC3)c1-c1nn(C)c3cnc(nc13)C2 ; Cn1cc(C(F)(F)F)nc1-c1ccc2c(c1)CCCOc1ncnc(C3CC3)c1-c1nn(C)c3cnc(nc13)C2]

The invention also encompasses a pharmaceutical composition comprising a compound of Formula I to XIX in admixture with one or more physiologically acceptable carriers or excipients.

The invention also encompasses a compound of Formula I to XIX or a pharmaceutically acceptable derivative thereof for use in human or veterinary medicine.

The invention also encompasses a method of treating a human or animal subject suffering from a condition which is mediated by the action of USP1, which method comprises administering to said subject an effective amount of a compound of Formula I to XIX.

The invention also encompasses the use of a compound of Formula I to XIX for the manufacture of a therapeutic agent for the treatment of a condition which is mediated by the action of USP1.

### Definitions

The term “alkyl” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C1-10 or C₁₋₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”. The alkyl is optionally substituted with one or more halogen atom(s).

The term “halogenated alkyl” means alkyl as defined above wherein one or more hydrogen atoms have been replaced by halogen atoms.

The term “alkenyl” means carbon chains which contain at least one carbon-carbon double bond, and which may be linear or branched, Z- or E-, or combinations thereof. Examples of alkenyl are CH₃CH═CH—, CH₂=CHCH₂— and CH(CH₃)₂CH═CH—.

The term “alkylene” by itself or as part of another substituent means a divalent radical derived from an alkyl, as exemplified, but not limited, by —CH₂CH₂CH₂CH₂—, —CH═CH—, —CH₂CH═CHCH₂—, —CH₂C≡CCH₂—, —CH₂CH₂CH(CH₂CH₂CH₃)CH₂—. The alkylene radical may be optionally substituted independently with one or more substituents described herein, and includes radicals having “cis” and “trans” orientations, or alternatively, “E” and “Z” orientations. Typically, an alkyl (or alkylene) group has from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The alkylene is optionally substituted with one or more halogen atom(s).

The term “alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like. The alkynyl is optionally substituted with one or more halogen atom(s).

The term “alkylamino” refers to an amino substituent which is further substituted with one or two alkyl groups.

The term “aminoalkyl” refers to an alkyl substituent which is further substituted with one or more amino groups. The term “hydroxyalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The term “cycloalkyl” or “carbococyclyl” means mono-, bicyclic or spiro-bicyclic carbocyclic rings, each of which has from 3 to 10 carbon atoms. A “fused analog” of cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, and the like. The spiro-bicyclic carbocyclic rings are bicyclic (having just two rings) or have a bicyclic portion as part of the larger ring system, in either case with the two rings connected through the defining single common carbon atom. The cycloalkyl is optionally substituted with one or more halogen atom(s).

The term “alkoxy” means alkoxy groups of a straight or branched having the indicated number of carbon atoms. C₁₋₆alkoxy, for example, includes methoxy, ethoxy, propoxy, isopropoxy, and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “cycloalkoxy” means cycloalkyl as defined above bonded to an oxygen atom, such as cyclopropyloxy.

The term “halogenated alkoxy” means alkoxy as defined above wherein one or more hydrogen atoms have been replaced by halogen atoms.

The term “aryl” means mono- or bicyclic aromatic rings containing only carbon atoms. A “fused analog” of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,4-benzodioxanyl, and the like.

The term “heteroaryl” means a mono- or bicyclic aromatic ring containing at least one heteroatom selected from N, O and S, with each ring containing 5 to 6 atoms. A “fused analog” of heteroaryl means a heteroaryl group fused to a monocyclic cycloalkyl or monocyclic heterocyclyl group in which the point of attachment is on the aromatic portion. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

The term “heterocyclyl” means mono-, bicyclic, tricyclic, spirocyclic, fused or bridged saturated ring systems containing at least one heteroatom selected from N, S and O, each of said ring having from 3 to 10 atoms in which the point of attachment may be carbon or nitrogen. A “heterocyclyl” includes a “fused analog” which means a monocyclic heterocycle fused to a heterocycle, a carbocycle, an aryl or heteroaryl group in which the point of attachment is on the non-aromatic portion. Examples of “heterocyclyl” and fused analogs thereof include azeridinyl, azetidinyl, pyrrolidinyl, piperidinyl, piperazinyl, imidazolidinyl, imidazopyridinyl, thiazolidinyl, dioxolanyl, oxazolodinyl, decahydroquinolinyl, piperidonyl, 4-piperidinonyl, quinuclidinyl, thiomorpholinyl, thiomorphonulyl 1,1-dioxide, morpholinyl, azapanyl, oxazepanyl, azabicyclohexanyls, azabicycloheptanyl, azabicyclooctanyl, azabycyclononanyl, azaspiroheptanyl, dihydro-1H,3H,5H-oxazolo[3,4-c]oxazolyl, tetrohydro-1′H3′Hspiro[cyclopropane-1,2′-pyrrolizinyl, hexahydro-1H-pyrralizinyl, hexahydro-1H-pyrrolo[2,1-c][1,4]oxazinyl, octahydroindolizinyl, oxaazaspirononanyls, oxaazspirooctanyls, diazaspirononanyls, oxaazabicycloheptanyls, hexahydropyrrolidinyl 4(1H-oxide, tetrohydro-2H-thiopyranyl 1-oxide, tetrohydro-2H-thiopyranyl 1,1-dioxide, bicycloheptanyl, 2,3-dihydrofuro(2,3-b)pyridyl, benzoxazinyl, tetrahydrohydroquinolinyl, tetrahydroisoquinolinyl, hexahydro-1H-pyrrolizine, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils).

The said alkyl groups, cycloalkyl groups, heterocyclyl groups, aryl groups and heteroaryl groups referred to in the definitions are unsubstituted or are substituted by at least one substituent selected from the group consisting of substituents. The substituents are selected from the group consisting of halogen atoms, alkyl groups having from 1 to 4 carbon atoms, alkoxy groups having from 1 to 4 carbon atoms, haloalkyl groups having from 1 to 4 carbon atoms, haloalkoxy groups having from 1 to 4 carbon atoms, cyano groups, alkynyl groups having from 2 to 6 carbon atoms, alkanoyl groups having from 1 to 5 carbon atoms, cycloalkyl groups having from 3 to 7 ring atoms, heteroaryl groups, aryl groups, aralkoxy groups having from 7 to 10 carbon atoms, arylcarbonyl groups, two adjacent-x groups are optionally joined together to form an alkylene or an alkenylene chain having 3 or 4 carbon atoms, aminocarbonyl groups, alkenyl groups having from 2 to 5 carbon atoms, alkylthio groups having from 1 to 4 carbon atoms, aminosulfinyl groups, aminosulfonyl groups, hydroxy groups, —SF₅, —P(O)Me₂, hydroxyalkyl groups having from 1 to 4 carbon atoms, nitro groups, amino groups, carboxy groups, alkoxycarbonyl groups having from 2 to 5 carbon atoms, alkoxyalkyl groups having from 1 to 4 carbon atoms, alkylsulfonyl groups having from 1 to 4 carbon atoms, alkanoylamino groups having from 1 to 4 carbon atoms, alkanoyl(alkyl)amino groups having from 1 to 6 carbon atoms, alkanoylaminoalkyl groups having from 1 to 6 carbon atoms in both the alkanoyl and alkyl part, alkanoyl(alkyl)aminoalkyl groups having from 1 to 6 carbon atoms in both the alkanoyl and each alkyl part, alkylsulfonylamino groups having from 1 to 4 carbon atoms, mono- or di-alkylaminocarbonyl groups having from 1 to 6 carbon atoms, mono- or di-alkylaminosulfinyl groups having from 1 to 6 carbon atoms, mono- or di alkylaminosulfonyl groups having from 1 to 6 carbon atoms, aminoalkyl groups having from 1 to 4 carbon atoms, mono- or di-alkylamino groups having from 1 to 6 carbon atoms, mono- or di-alkylaminoalkyl groups having from 1 to 6 carbon atoms in each alkyl part, aralkyl groups having from 7 to 10 carbon atoms, heteroarylalkyl groups having from 1 to 4 carbon atoms in the alkyl part, heteroarylalkoxy groups having from 1 to 4 carbon atoms in the alkoxy part and alkylsulfonylamino groups having from 1 to 4 carbon atoms.

The terms “halo” or “halogen,” by themselves or as part of another substituent, mean, unless otherwise stated, a fluorine, chlorine, bromine, or iodine atom. Additionally, terms such as “haloalkyl,” or “halogenated alkyl” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrugs may also have improved solubility in pharmaceutical compositions over the parent drug. An example, without limitation, of a prodrug would be a compound of any of Formula I, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

### Optical Isomers-Diastereomers-Geometric Isomers-Tautomers

Compounds of the present invention may contain one or more asymmetric centers and may thus occur as racemates and racemic mixtures, single enantiomers, diastereomeric mixtures and individual diastereomers. The present invention is meant to comprehend all such isomeric forms of the compounds.

Some of the compounds described herein contain olefinic double bonds, and unless specified otherwise, are meant to include both E and Z geometric isomers.

Some of the compounds of described herein may contain one or more than one cyclic ring systems and may thus exist in cis- and trans-isomers. The present invention is meant to include all such cis- and trans-isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of the present invention.

Compounds described herein may be separated into diastereoisomeric pairs of enantiomers by, for example, fractional crystallization from a suitable solvent, for example MeOH or EtOAc or a mixture thereof. The pair of enantiomers thus obtained may be separated into individual stereoisomers by conventional means, for example by the use of an optically active amine or acid as a resolving agent or on a chiral HPLC column.

Alternatively, any enantiomer of a compound described herein may be obtained by stereospecific synthesis using optically pure starting materials or reagents of known configuration.

### Stable Isotope-Labeled Analogs

One or more than one of the protons (hydrogen atoms) in compounds of the present invention can be replaced with deuterium atom(s), thus providing deuterated analogs that may have improved pharmacokinetic properties or pharmacological activities.

### Salts and Formulations

The compounds described herein can be useful as the free base or as a salt.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydramine, isopropylamine, lysine, methylglucamine, morpholine, piperazine, piperidine, polyamine resins, procaine, purines, theobromine, triethylamine, trimethylamine, tripropylamine, tris(hydroxymethyl)aminomethane, tromethamine, and the like.

When the compound of the present invention is alkaline, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of the present invention are meant to also include the pharmaceutically acceptable salts.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients are mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or 122yridazine suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an I atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns).

This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 mg to 20 mg of the compound of the invention per actuation and the actuation volume may vary from 1 L to 100 L. A typical formulation may comprise a compound of Formula (A) propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 1 pg to 1 mg of the compound of the present invention. The overall daily dose will typically be in the range 1 pg to 1 mg which may be administered in a single dose or, more usually, as divided doses throughout the day.

Compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of the present invention are employed. (For purposes of this application, topical application shall include mouth washes and gargles.) Dosage levels of the order of from about 0.01 mg to about 140 mg/kg of body weight per day are useful in the treatment of the above-indicated conditions, or alternatively about 0.5 mg to about 7 g per patient per day. For example, a condition may be effectively treated by the administration of from about 0.01 to 50 mg of the compound per kilogram of body weight per day, or alternatively about 0.5 mg to about 3.5 g per patient per day, preferably 2.5 mg to 1 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

### Utilities

The compounds of the invention are inhibitors of USP1 and are useful in treating a USP1 associated disease or condition such as cancer.

The invention also encompasses a method of treating cancer with an effective amount of a compound of the present invention or using a combination of an effective amount of a compound of the present invention with an effective amount of radiation, chemotherapies, PARP inhibitors, SHP2 inhibitors, FAK inhibitors, endocrine/hormone therapeutics, immunotherapies such as antibodies against programmed cell death protein 1 (PD-1), programmed death ligand 1 (PD-L1), or cytotoxic t-lymphocyte antigen 4 (CTLA4).

In yet another aspect of the invention, the cancer treated is selected from ovarian cancers, breast cancers, pancreatic cancers, prostate cancers, gastric cancers, liver cancers, lung cancers, bladder cancers, skin cancers and other cancers.

### Synthesis

The compounds of the present invention can be prepared according to the following synthetic schemes:

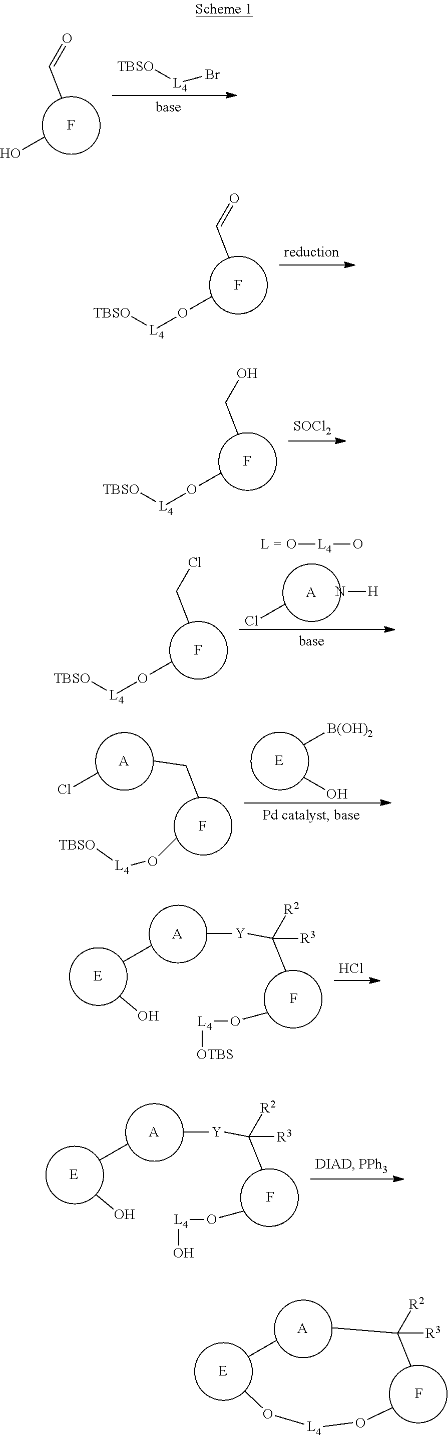

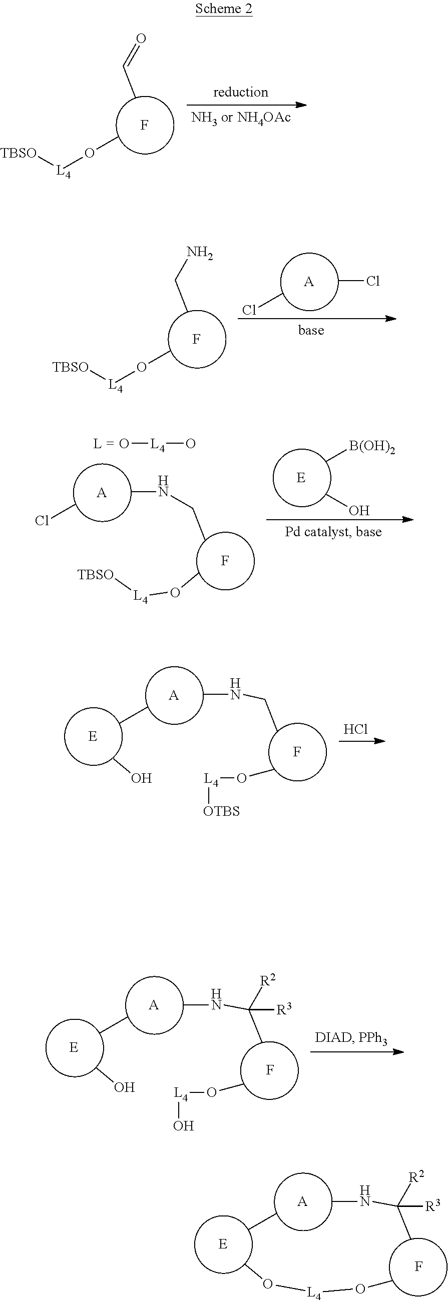

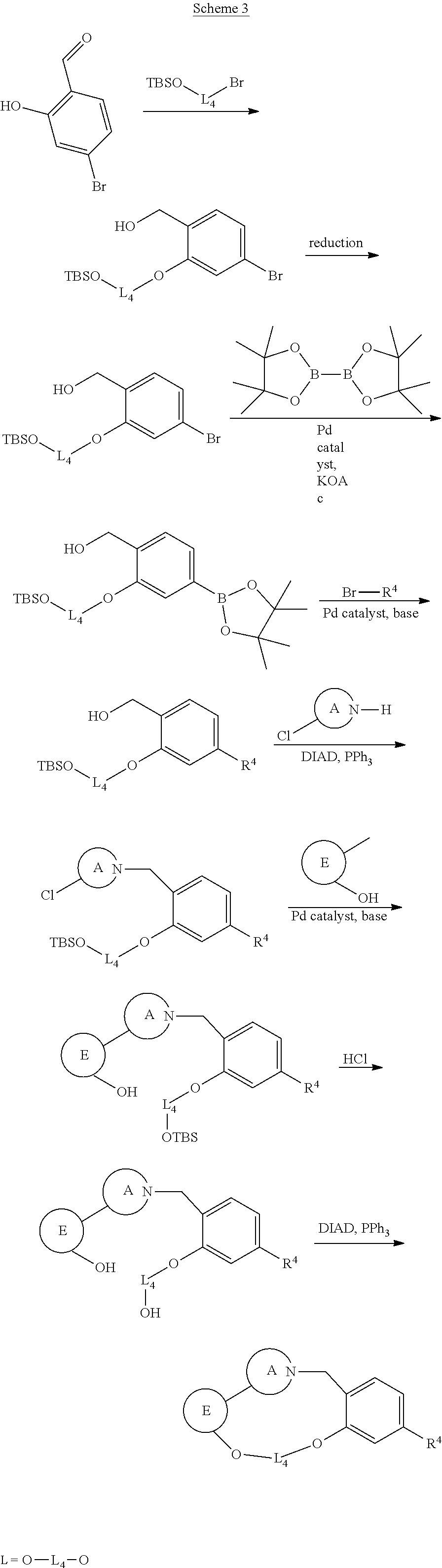

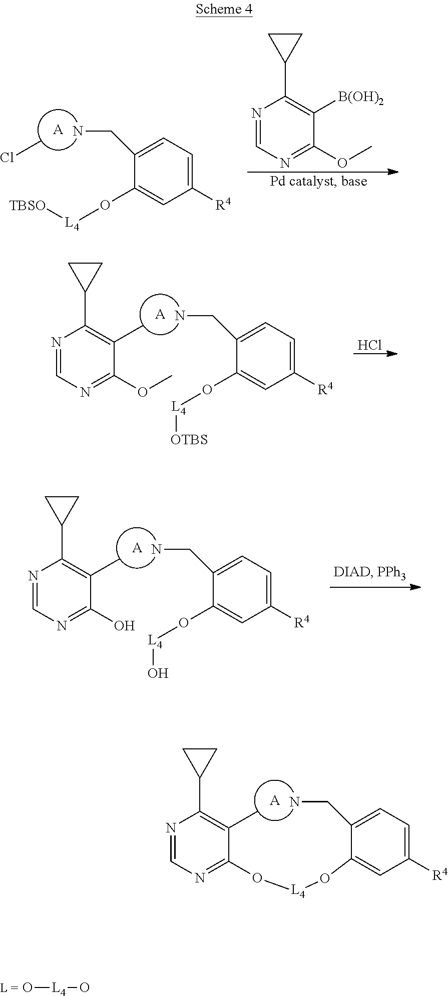

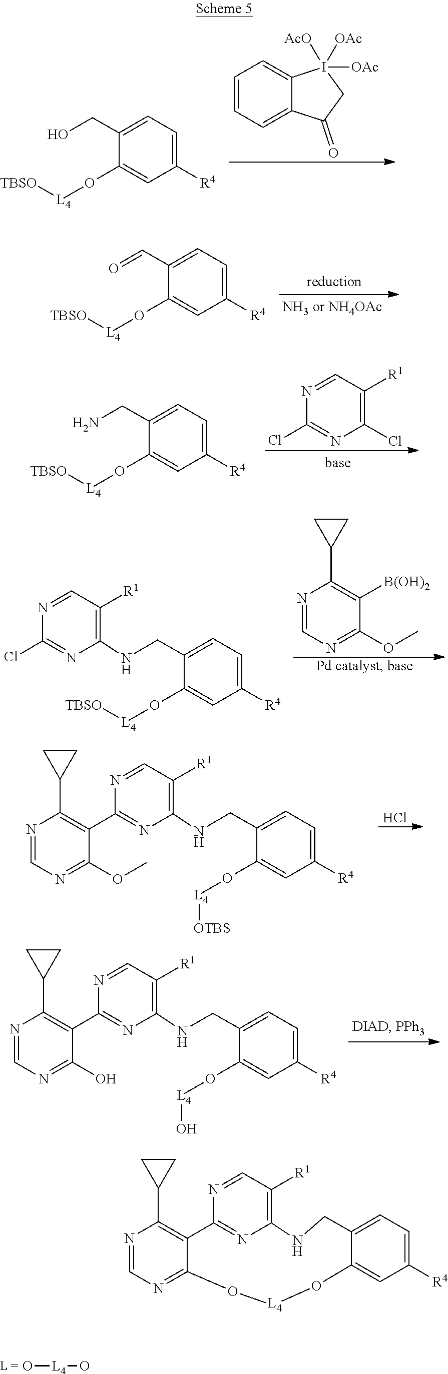

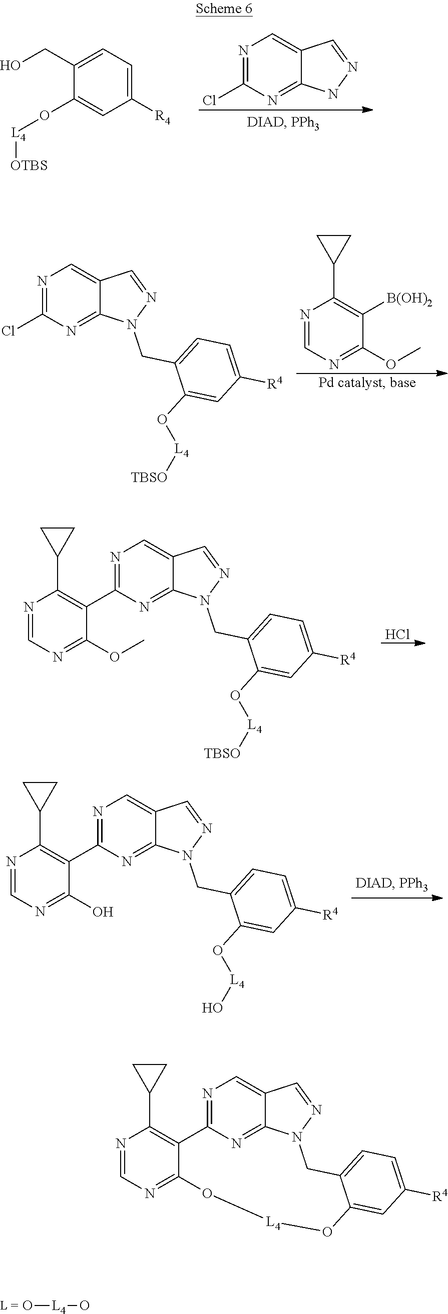

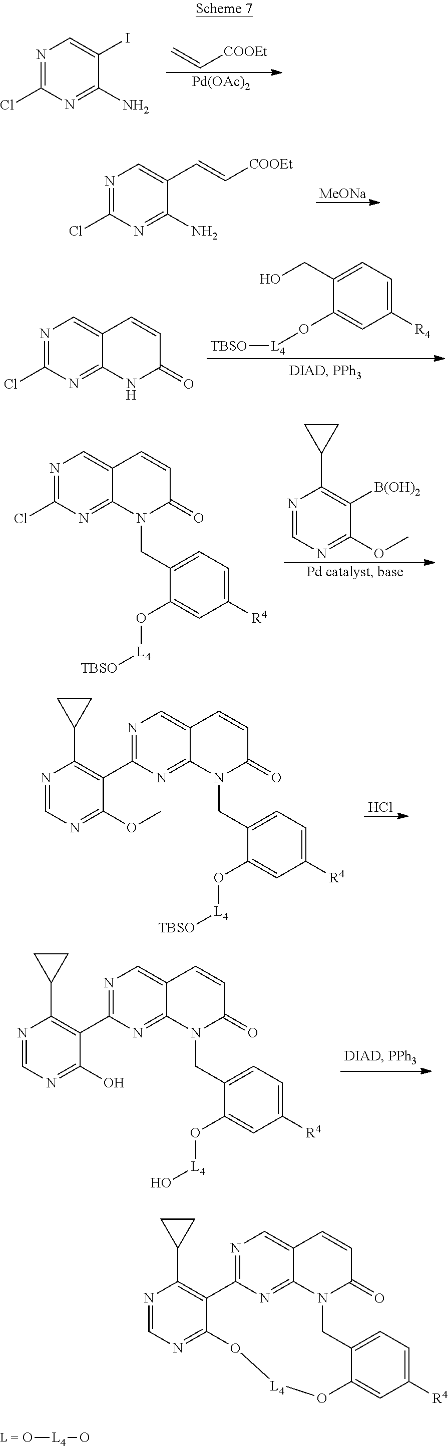

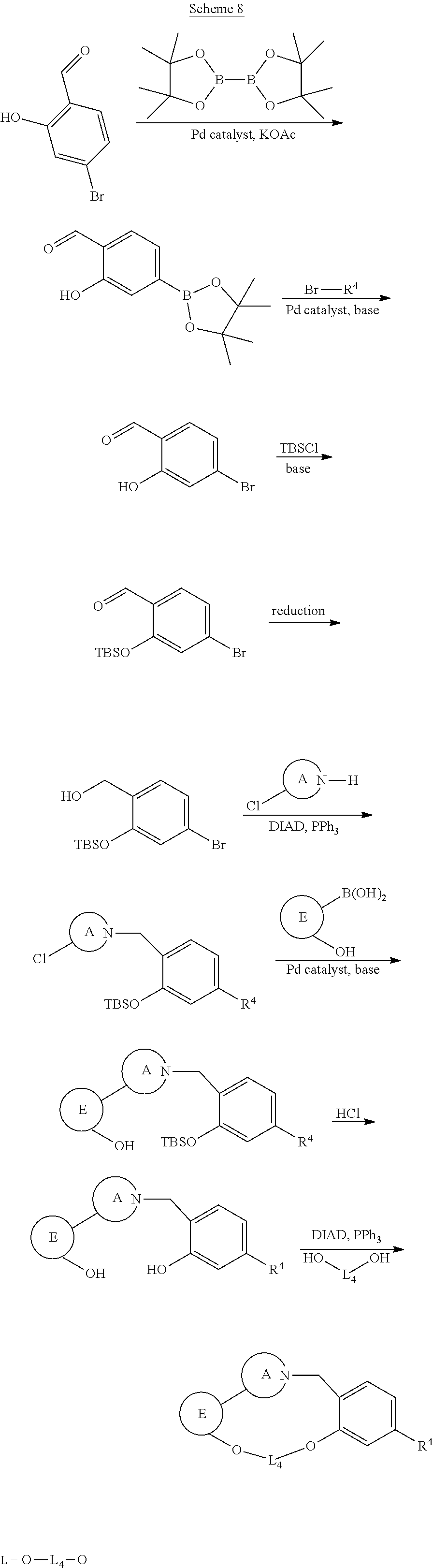

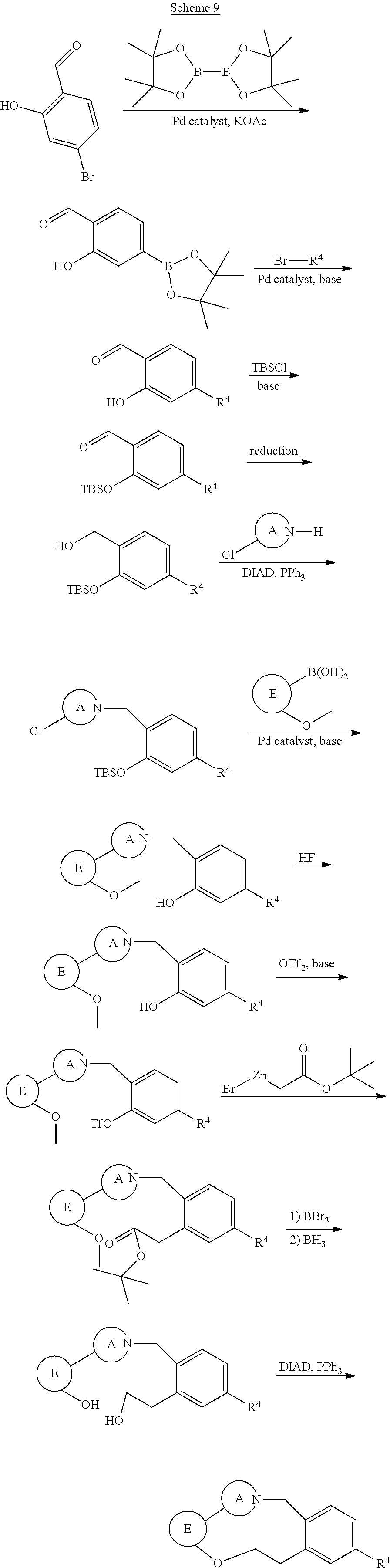

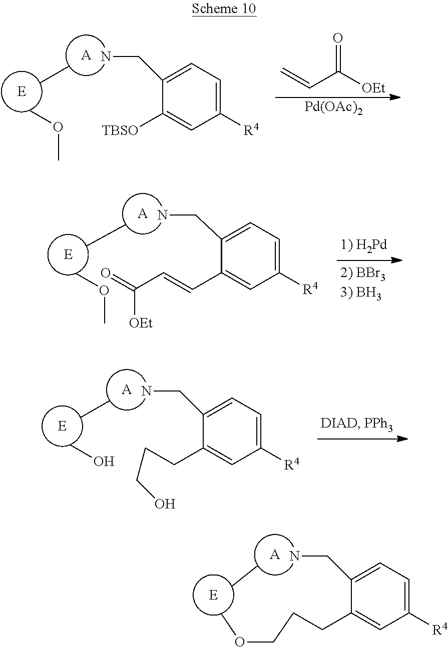

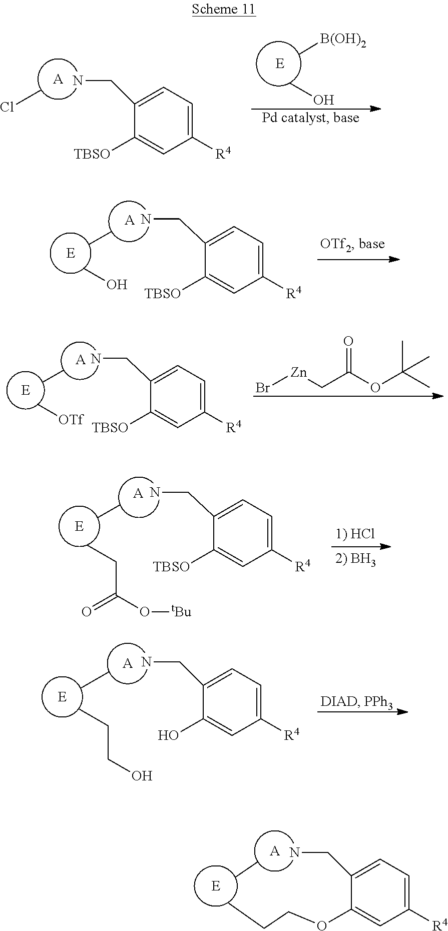

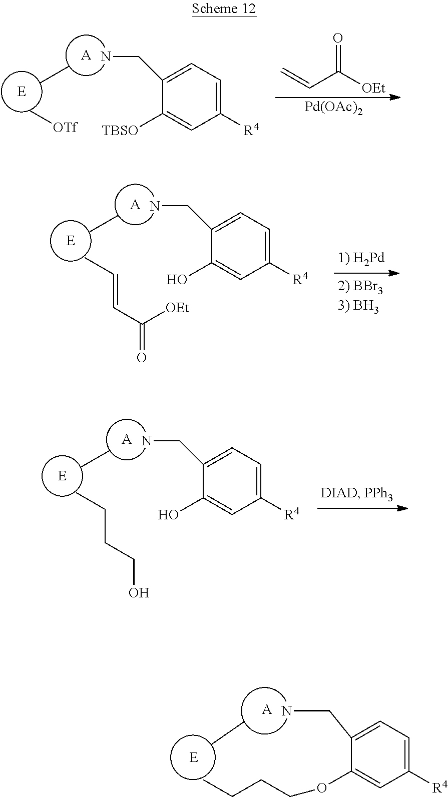

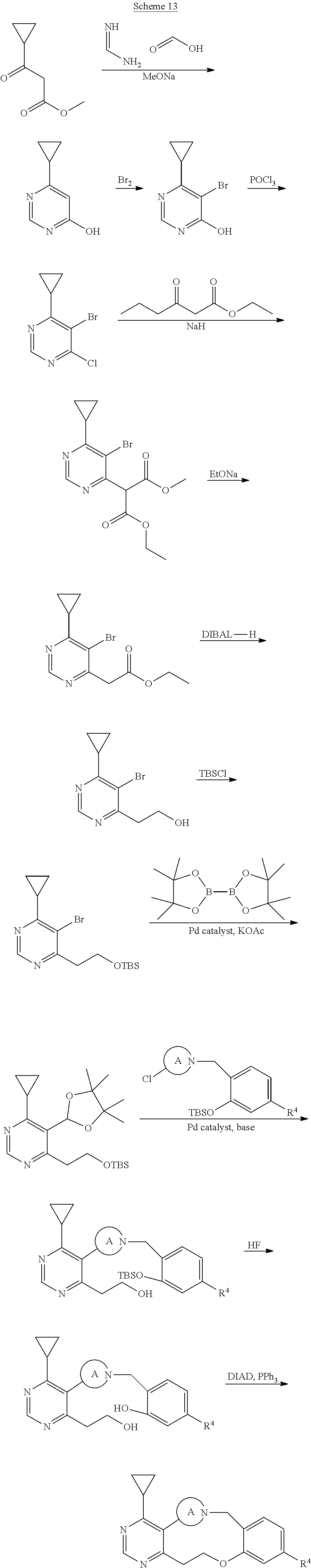

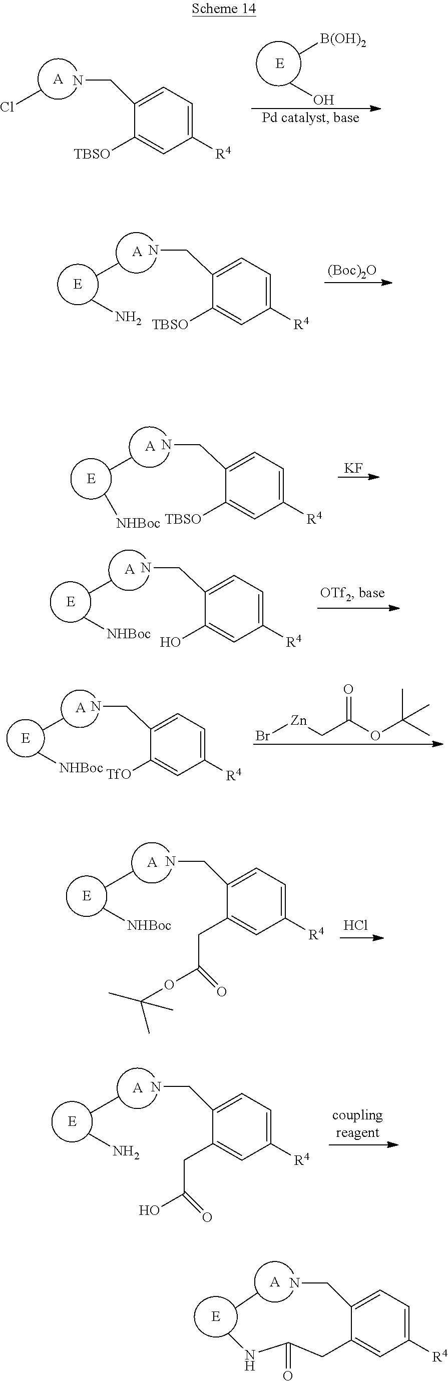

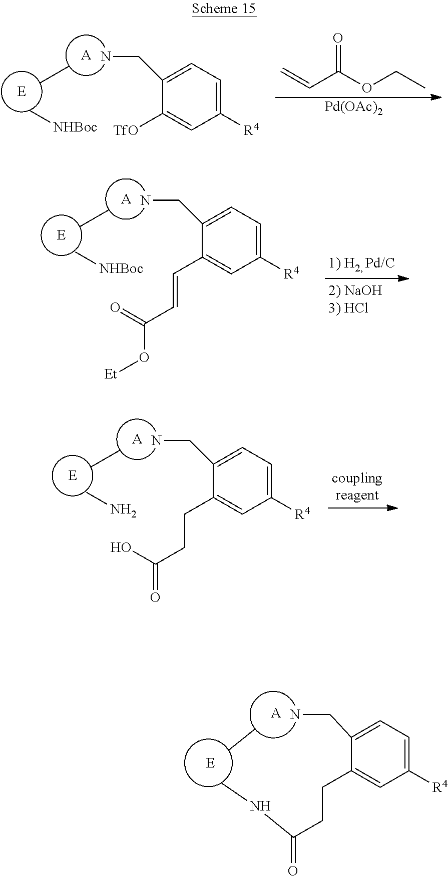

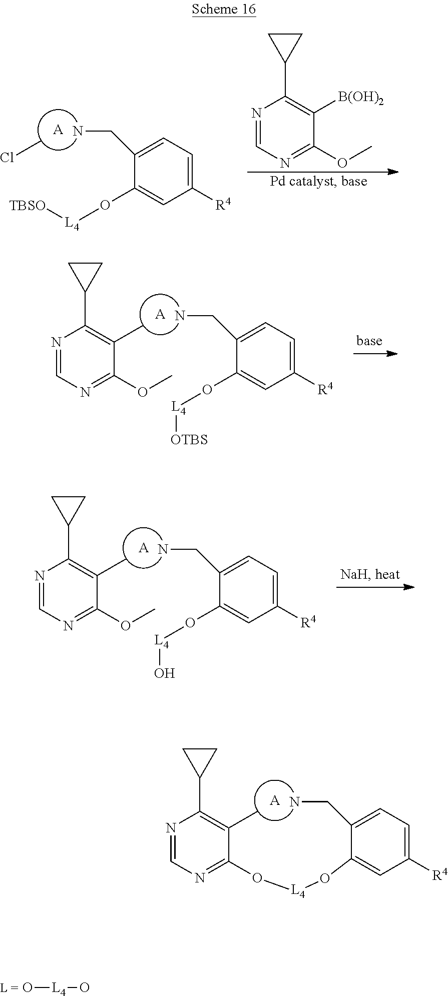

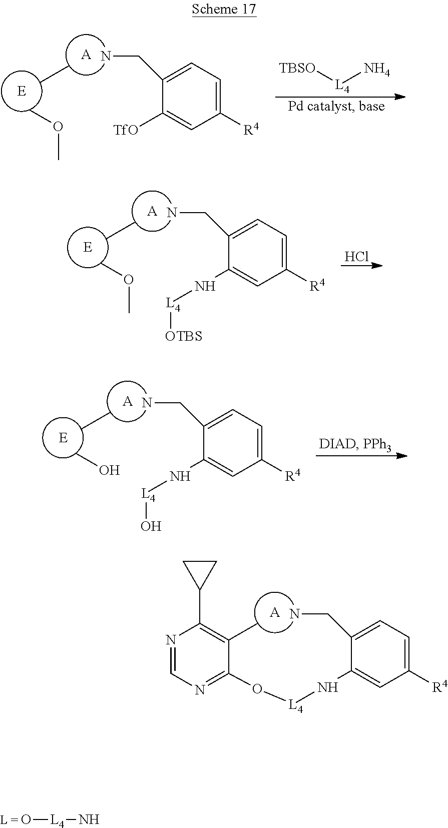

### Evaluation of Biological Activity

Deubiquitination assay for USP1/UAF1: compounds described here were tested for their ability to inhibit USP1 deubiquitinase activity in this assay using ubiquitin-rhodamine 110 as the substrate. The assay was conducted in the assay buffer (50 mM Tris-HCl, pH 7.8, 0.5 mM EDTA, 0.1 mg/ml Bovine Serum Albumin, 1 mM DTT, 0.01% Tween-20) in 384 well plates. 0.5 nM USP1/UAF1 (R&D Systems) was incubated with compounds in dose response format for 10 minutes at room temperature. Then the reaction was initiated by addition of 150 nM ubiquitin-rhodamine (R&D Systems). The fluorescence intensity was read after 45 minutes at Ex488/Em535 by CLARIOstar (BMG Labtech). IC₅₀ response curves were plotted.

Table 1 shows the activities of selected compounds of this invention in the USP1/UAF1 deubiquitination assay. The IC₅₀ value was determined as the concentration for 50% inhibition of the USP1/UAF1 activity compared to DMSO control (A: IC₅₀≤100 nM; B: IC₅₀ between 100 nM and 1,000 nM; C: IC₅₀≥1,000 nM; ND: not determined).

TABLE 1
Activities in USP1/UAF1 deubiquitination assay

| Example | IC50 |
|---|---|
| 1 | A |
| 2 | A |
| 3 | C |
| 4 | C |
| 5 | A |
| 6 | B |
| 7 | A |
| 8 | A |
| 9 | A |
| 10 | A |
| 11 | A |
| 12 | B |
| 13 | A |
| 14 | A |
| 15 | C |
| 16 | A |
| 17 | A |
| 18 | C |
| 19 | B |
| 20 | A |
| 21 | A |
| 22 | A |
| 23 | C |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | B |
| 40 | A |
| 41 | A |
| 42 | A |
| 43 | B |
| 44 | A |
| 45 | A |
| 46 | A |
| 47 | A |
| 48 | B |
| 49 | A |
| 50 | A |
| 51 | A |
| 52 | A |
| 53 | A |
| 54 | A |
| 55 | A |
| 56 | A |
| 57 | A |

### Abbreviations

The following abbreviations have the meanings indicated. EA means ethyl acetate; CIP means 2-chloro-1,3-dimethylimidazolidium hexafluorophosphate; DBU means 1,8-diazabicyclo[5.4.0]undec-7-ene; DIAD means diisopropyl azodicarboxylate; DIBAL or DIBAL-H means diisobutylaluminum hydride; DCM means dichloromethane; DDQ means 1,2-dichloro-4,5-dicyanobenzoquinone; DEAD means diethyl azodicarboxylate; DIAD means diisopropyl azodicarboxylate; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMA means N,N-Dimethylacetamide, DMSO means dimethylsulfoxide; dppb means 1,4-bis(diphenylphosphino)butane; dppe means 1,2-bis(diphenylphosphino)ethane; dppf means 1,1′-bis(diphenylphosphino)ferrocene; dppm means 1,1′-bis(diphenylphosphino)methane; EDCI means 1-(3-dimethylaminopropyl)-3-ethylcarbodiimide; HATU means 2-(7-Aza-1H-benzotriazole-1-yl)-1,1,3,3-tetramethyluronium hexafluorophosphate; HMPA means hexamethyl phosphorarnide; IPA means isopropyl alcohol; LDA means lithium diisopropylamide; LHMDS means lithium bis(hexamethyldisilylamide); LAH means lithium aluminum hydride; MCPBA means 3-chloroperbenzoic acid; MTBE means methyl tert-butyl ether; NBS means N-Bromosuccinimide; NCS means N-chlorosuccinimide; NMI means 1-methylimidazole; NMM means N-methylmorpholine; NMP means N-methylpyrrolidine; PE means petroleum ether; PyBOP 130yridazine130leriazol-1-yloxytripyrrolidinophosphonium hexafluorophosphate; SGCC means silica gel column chromatography; TBAF means (n-Butyl)₄NF; TDA means tris(2-(2-methoxyethoxy)ethyl)amine; TEA means triethylamine; TFA means trifluoroacetic acid; THF means tetrahydrofuran; PPh3 means triphenylphosphine; rt or r.t. means room temperature; sat. means saturated; T3P means propylphosphonic anhydride; TCFH means N,N,N′,N′-tetramethylchloroformaidinium hexafluorophosphate; * in structures means undetermined R or S chiral center; Pd₂(dba)₃ means tris(dibenzylideneacetone)dipalladium; X—PHOS means 2-(dicyclohexylphosphino)-2′,4′,6′-tri-i-propyl-1,1′-biphenyl.

### Lcms Conditions:

Column: Xtimate C18, 3 mm, 4.6×50 mm.
Mobile phase: 0.03% TFA in water and 0.03% TFA in CH₃CN;
Gradient: 5% CH₃CN to 95% CH₃CN in 6.5 min;
Flow rate: 1 mL/min.;
MS instrument: Waters UPLC-PDA-3100 (SQD3100) single-quadrupole mass spectrometer.

The present invention will be more readily understood by referring to the following examples which are given to illustrate the invention rather than to limit its scope.

### Example 1

### 1⁶-cyclopropyl-5⁴-(1-isopropyl-4-(trifluoromethyl)-1H-imidazol-2-yl)-2⁵-methyl-6,9-dioxa-3-aza-1(5,4),2(2,4)-dipyrimidina-5(1,2)-benzenacyclononaphane

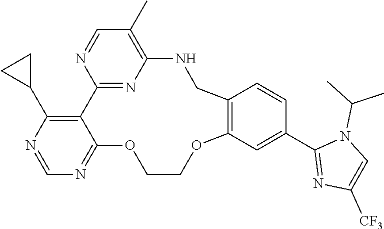 [SMILES: Cc1cnc2nc1NCc1ccc(-c3nc(C(F)(F)F)cn3C(C)C)cc1OCCOc1ncnc(C3CC3)c1-2]

### Step 1 4-bromo-2-(2-((tert-butyldimethylsilyl)oxy)ethoxy)benzaldehyde

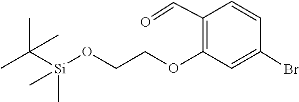 [SMILES: CC(C)(C)[Si](C)(C)OCCOc1cc(Br)ccc1C=O]

A mixture of 4-bromo-2-hydroxybenzaldehyde (1.5 g), (2-bromoethoxy)(tert-butyl)dimethylsilane (3.57 g), potassium iodide (1.86 g) and Cs₂CO₃ (7.29 g) in DMF (20 mL) was stirred at 50° C. for 4 h. The reaction solution was then poured into water (100 mL) and extracted with EtOAc (100 mL×2). The combine organic layer was washed with brine (100 mL) and dried over Na₂SO₄ and concentrated under vacuum. The residue was purified by SGCC eluted with EtOAc/PE (0 to 40% EtOAc) to afford the title product as a white solid (2.6 g).

### Step 2 2-(2-((tert-butyldimethylsilyl)oxy)ethoxy)-4-(4,4,5,5-tetramethyl-1,3,2-dioxaborolan-2-yl)benzaldehyde

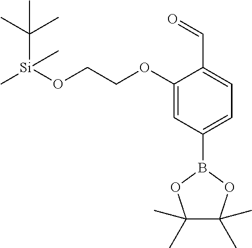 [SMILES: CC1(C)OB(c2ccc(C=O)c(OCCO[Si](C)(C)C(C)(C)C)c2)OC1(C)C]

A mixture of the product of Step 1 (1.4 g), 4,4,4′,4′,5,5,5′,5′-octamethyl-2,2′-bi(1,3,2-dioxaborolane) (1.98 g), KOAc (1.91 g) and [Pd(dppf)Cl₂ (200 mg) in 1,4-dioxane (20 mL) was degassed under vacuum and purged with Ar three times. The reaction mixture was stirred at 100° C. for 3 h. The reaction solution was concentrated and then purified by SGCC eluted with EtOAc/PE (0% to 50% EtOAc) to afford the title product as a white solid (1.4 g). LCMS (ESI): 407.53 [M+1]⁺.

### Step 3 1-isopropyl-4-(trifluoromethyl)-1H-imidazole

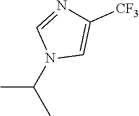

To a mixture of 4-(trifluoromethyl)-1H-imidazole (5.0 g) and Cs₂CO₃ (13.2 g) in anhydrous DMF (100 mL) at 0° C. was added 2-iodopropane (6.8 g). The reaction mixture was then warmed to rt and stirred overnight. The reaction mixture was poured into 100 mL ice-water, extracted by 3×50 mL EtOAc. The combined organic layer was washed with 50 mL saturated brine, dried over anhydrous Na₂SO₄ and concentrated. The residue was purified by SGCC (PE/EtOAc: from 100:0 to 10:1) to afford the title compound as white solids (4.2 g). LCMS (ESI): 179.3 [M+1]⁺.

### Step 4 2-bromo-1-isopropyl-4-(trifluoromethyl)-1H-imidazole

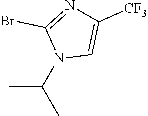

To a stirred solution of the product of Step 3 (500 mg, 2.806 mmol) in THF (25 mL) at −78° C., was added n-butyllithium (2.5 M in hexane, 1.5 mL) dropwise. The resulting mixture was stirred for 30 min at −78° C. and then treated with a solution of carbon tetrabromide (691 mg, 4.210 mmol) in THF (10 mL). The reaction mixture was allowed to warm room temperature and then stirred for 16 h. The reaction mixture was quenched with saturated ammonium chloride solution and extracted with EtOAc (3×50 mL). The combined organic layer was dried over anhydrous Na₂SO₄, filtered and concentrated under reduced pressure. The crude product was purified by SGCC eluted with 0-10% EtOAc in hexane to afford the title compound (368 mg). LCMS (ESI): 257.15 [M+1]⁺.

### Step 5 2-(2-((tert-butyldimethylsilyl)oxy)ethoxy)-4-(1-isopropyl-4-(trifluoromethyl)-1H-imidazol-2-yl) benzaldehyde

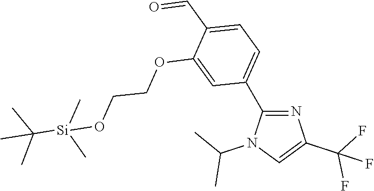 [SMILES: CC(C)n1cc(C(F)(F)F)nc1-c1ccc(C=O)c(OCCO[Si](C)(C)C(C)(C)C)c1]

A mixture of the product of Step 2 (1.4 g), [2′-(amino-κN)[1,1′-biphenyl]-2-yl-κC]chloro [dicyclohexyl[2′,4′,6′-tris(1-methylethyl)[1,1′-biphenyl]-2-yl]phosphine]palladium (306 mg), K₃PO₄ (1.65 g) and the product of Step 4 (1.0 g) in 1,4-dioxane/water (20 mL, 8/1) was degassed under vacuum and purged with Ar three times. The mixture was stirred at 70° C. for 3 h. The reaction solution was evaporated under reduced pressure and purified by silica gel chromatography eluted with EtOAc/PE (0% to 100% EtOAc) to afford the title product as a white solid (800 mg). LCMS (ESI): 457.56 [M+1]⁺.

### Step 6 (2-(2-((tert-butyldimethylsilyl)oxy)ethoxy)-4-(1-isopropyl-4-(trifluoromethyl)-1H-imidazol-2-yl)phenyl) methanamine

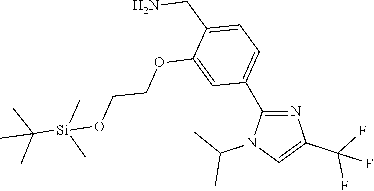 [SMILES: CC(C)n1cc(C(F)(F)F)nc1-c1ccc(CN)c(OCCO[Si](C)(C)C(C)(C)C)c1]

A mixture of the product of Step 5 (300 mg), ammonium formate (2.0 g) and Pd/C (63 mg, 10% by weight) in methanol (30 mL) was stirred at 70° C. overnight. The reaction solution was filtered and washed with methanol (20 mL). The combined organic layer was concentrated under vacuum and the residue was dissolved into DCM (20 mL). The mixture was filtered and washed with DCM (20 mL). The organic layer was concentrated to afford the title product as a white solid (300 mg). LCMS (ESI): 458.70 [M+1]⁺.

### Step 7 N-(2-(2-((tert-butyldimethylsilyl)oxy)ethoxy)-4-(1-isopropyl-4-(trifluoromethyl)-1H-imidazol-2-yl) benzyl)-2-chloro-5-methylpyrimidin-4-amine

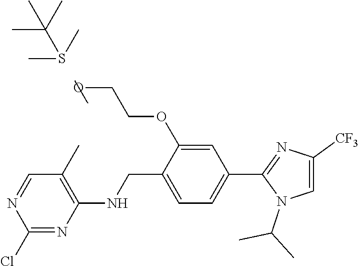

A mixture of the product of Step 6 (300 mg), 2,4-dichloro-5-methylpyrimidine (107 mg) and i-Pr₂NEt (1.0 g) in 1,4-dioxane (3.0 mL) was stirred at 80° C. overnight. The mixture was cooled to room temperature and then extracted with water (15.0 mL) and EtOAc (15.0 mL×3). The organic layer was washed with brine (20 mL), dried over anhydrous Na₂SO₄, concentrated under vacuum. The residue was purified by SGCC eluted with EtOAc/PE (0% to 100% EtOAc) to afford the title product as a white solid (240 mg). LCMS (ESI): 584.26 [M+1]⁺.

### Step 8 N-(2-(2-((tert-butyldimethylsilyl)oxy)ethoxy)-4-(1-isopropyl-4-(trifluoromethyl)-1H-imidazol-2-yl) benzyl)-4′-cyclopropyl-6′-methoxy-5-methyl-[2,5′-bipyrimidin]-4-amine

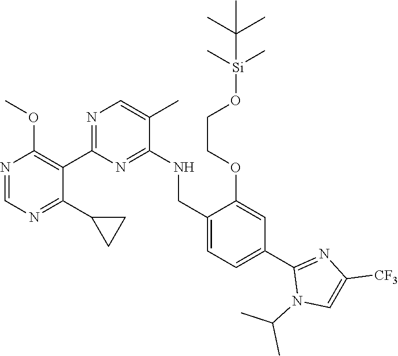 [SMILES: COc1ncnc(C2CC2)c1-c1ncc(C)c(NCc2ccc(-c3nc(C(F)(F)F)cn3C(C)C)cc2OCCO[Si](C)(C)C(C)(C)C)n1]

A mixture of the product of Step 7 (240 mg), (4-cyclopropyl-6-methoxypyrimidin-5-yl)boronic acid (159 mg), CsF (187 mg), Pd₂(dba)₃ (60 mg) and 2-(dicyclohexylphosphino)-2′,4′,6′-tri-i-propyl-1,1′-biphenyl (68 mg) in 1,4-dioxane/water (4 mL, v/v: 8/1) was degassed under vacuum and purged with Ar three times. The reaction mixture was stirred at 90° C. for 6 h. The solvent was evaporated under reduced pressure and the residue was purified by SGCC with EtOAc/PE (0% to 100% EtOAc) to afford the title product as a white solid (150 mg). LCMS (ESI): 698.78 [M+1]⁺.

### Step 9 2-(2-(((4′-cyclopropyl-6′-methoxy-5-methyl-[2,5′-bipyrimidin]-4-yl)amino)methyl)-5-(1-isopropyl-4-(trifluoromethyl)-1H-imidazol-2-yl)phenoxy)ethan-1-ol

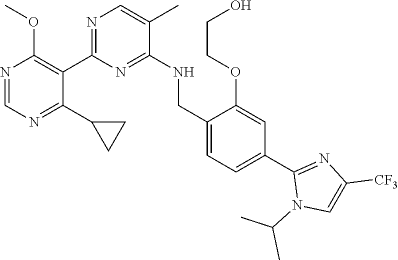 [SMILES: COc1ncnc(C2CC2)c1-c1ncc(C)c(NCc2ccc(-c3nc(C(F)(F)F)cn3C(C)C)cc2OCCO)n1]

A solution of the product of Step 8 (150 mg) in TFA (2 mL) was stirred at room temperature for 1 h. The reaction solution was poured into saturated NaHCO₃ aqueous (10 mL) (pH=8). The resulting mixture was extracted with EtOAc (20 mL×3). The organic layer was washed with brine (10 mL), dried over anhydrous Na₂SO₄ and then concentrated under vacuum to afford the title product as a white solid (120 mg). LCMS (ESI): 584.56 [M+1]⁺.

### Step 10 1⁶-cyclopropyl-5⁴-(1-isopropyl-4-(trifluoromethyl)-1H-imidazol-2-yl)-2⁵-methyl-6,9-dioxa-3-aza-1(5,4),2(2,4)-dipyrimidina-5(1,2)-benzenacyclononaphane

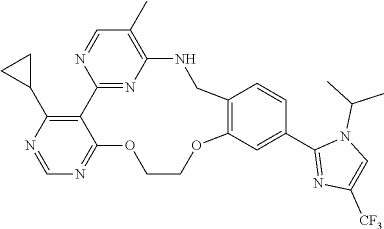 [SMILES: Cc1cnc2nc1NCc1ccc(-c3nc(C(F)(F)F)cn3C(C)C)cc1OCCOc1ncnc(C3CC3)c1-2]

To a solution of the product of Step 9 (40 mg) in 1,4-dioxane (2 mL) was added NaH (41 mg) at room temperature. The mixture was stirred at 90° C. for 3 h. The reaction solution was cooled to 0° C. and quenched by acetic acid (0.1 mL). The mixture was poured into water (10 mL) and extracted with EtOAc (10 mL×2). The organic layer was washed with brine (10 mL), dried over anhydrous Na₂SO₄ and then concentrated under vacuum. The residue was purified by Prep-TLC with EtOAc/PE (2/1) to afford the title product as a white solid (5.0 mg). LCMS (ESI): 552.53 [M+1]⁺. ¹H NMR (500 MHz, DMSO-d₆) δ 8.64 (s, 1H), 8.17 (s, 1H), 8.02 (s, 1H), 7.74 (d, 1H), 7.27 (br, 1H), 7.15 (d, 1H), 7.12 (s, 1H), 5.3-4.5 (m, 5H), 4.37 (s, 2H), 2.04-1.96 (m, 4H), 1.42 (d, 6H), 1.10-1.03 (m, 2H), 0.98-0.92 (m, 2H).

### Example 2

### 1⁶-cyclopropyl-4⁴-(1-isopropyl-4-(trifluoromethyl)-1H-imidazol-2-yl)-2¹H-5,8-dioxa-2(6,1)-pyrazolo[3,4-d]pyrimidina-1(5,4)-pyrimidina-4(1,2)-benzenacyclooctaphane

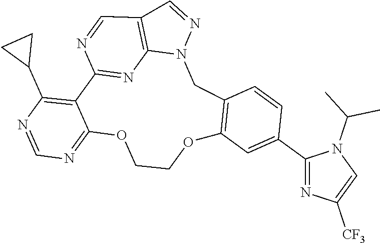 [SMILES: CC(C)n1cc(C(F)(F)F)nc1-c1ccc2c(c1)OCCOc1ncnc(C3CC3)c1-c1ncc3cnn(c3n1)C2]

### Step 1 (2-(2-((tert-butyldimethylsilyl)oxy)ethoxy)-4-(1-isopropyl-4-(trifluoromethyl)-1H-imidazol-2-yl)phenyl) methanol

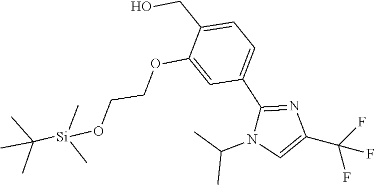 [SMILES: CC(C)n1cc(C(F)(F)F)nc1-c1ccc(CO)c(OCCO[Si](C)(C)C(C)(C)C)c1]

To a solution of the product of Step 5 in Example 1 (500 mg) in methanol (10 mL) was added sodium borohydride (50 mg) at 0° C. The reaction was stirred at 0° C. for 1 h. The reaction was quenched by saturated ammonium chloride aqueous (1 mL) and then concentrated under vacuum. The residue was purified by SGCC eluted with EtOAc/PE (0% to 100% EtOAc) to afford the title product as a colorless oil (400 mg). LCMS (ESI): 459.58 [M+1]⁺.

### Step 2 1-(2-(2-((tert-butyldimethylsilyl)oxy)ethoxy)-4-(1-isopropyl-4-(trifluoromethyl)-1H-imidazol-2-yl) benzyl)-6-chloro-1H-pyrazolo[3,4-d]pyrimidine

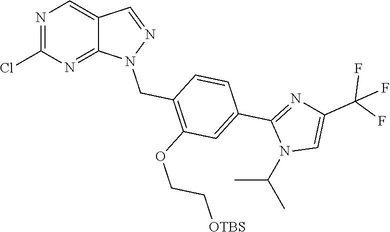 [SMILES: CC(C)n1cc(C(F)(F)F)nc1-c1ccc(Cn2ncc3cnc(Cl)nc32)c(OCCO[Si](C)(C)C(C)(C)C)c1]

To a solution of the product of Step 1 (356 mg), 6-chloro-1H-pyrazolo[3,4-d]pyrimidine (120 mg) and tributylphosphine in THF (3 mL) was added dropwise DIAD (220 mg) at room temperature under Ar atmosphere. The reaction mixture was stirred at room temperature for 2 h. The reaction solution was concentrated and the residue was purified by SGCC eluted with EtOAc/PE (0% to 100% EtOAc) to afford the title product as a white solid (360 mg). LCMS (ESI): 595.26 [M+1]⁺.

### Step 3 1-(2-(2-((tert-butyldimethylsilyl)oxy)ethoxy)-4-(1-isopropyl-4-(trifluoromethyl)-1H-imidazol-2-yl) benzyl)-6-(4-cyclopropyl-6-methoxypyrimidin-5-yl)-1H-pyrazolo[3,4-d]pyrimidine

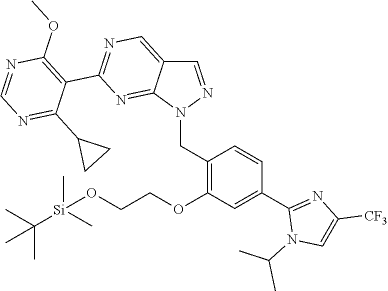 [SMILES: COc1ncnc(C2CC2)c1-c1ncc2cnn(Cc3ccc(-c4nc(C(F)(F)F)cn4C(C)C)cc3OCCO[Si](C)(C)C(C)(C)C)c2n1]

A mixture of the product of Step 2 (340 mg), (4-cyclopropyl-6-methoxypyrimidin-5-yl)boronic acid (177 mg), CsF (187 mg), Pd2(dba)3 (60 mg) and 2-(dicyclohexylphosphino)-2′,4′,6′-tri-i-propyl-1,1′-biphenyl (68 mg) in 1,4-dioxane/water (4 mL, v/v: 8/1) was degassed under vacuum and purged with Ar three times. After stirring at 90° C. overnight, the reaction mixture was evaporated under reduced pressure and the residue was purified by SGCC eluted with EtOAc/PE (0% to 100% EtOAc) to afford the title product as a white solid (240 mg). LCMS (ESI): 709.36 [M+1]⁺.

### Step 4 6-cyclopropyl-5-(1-(2-(2-hydroxyethoxy)-4-(1-isopropyl-4-(trifluoromethyl)-1H-imidazol-2-yl)benzyl)-1H-pyrazolo[3,4-d]pyrimidin-6-yl)pyrimidin-4-ol

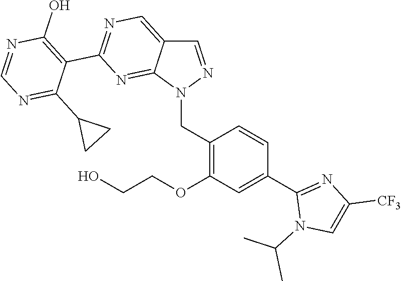 [SMILES: CC(C)n1cc(C(F)(F)F)nc1-c1ccc(Cn2ncc3cnc(-c4c(O)ncnc4C4CC4)nc32)c(OCCO)c1]

A solution of the product of Step 3 (180 mg) in TFA/water (10 mL, v/v: 9/1) was stirred at 80° C. for 8 h. The reaction mixture was evaporated under reduced pressure and then the residue was dissolved into methanol/water (5 mL, v/v: 4/1). To the above solution was added K₂CO₃ (351 mg) at room temperature. The reaction mixture was stirred at room temperature for 2 h, and then purified by C18 column chromatography (grain size: 20-45 m, pore size distribution: 100 Å) eluted with water (0.1% formic acid)/acetonitrile (0 to 70% acetonitrile) to afford the title product as a white solid (100 mg). LCMS (ESI): 581.42 [M+1]⁺.

### Step 5 1⁶-cyclopropyl-4⁴-(1-isopropyl-4-(trifluoromethyl)-1H-imidazol-2-yl)-2¹H-5,8-dioxa-2(6,1)-pyrazolo[3,4-d]pyrimidina-1(5,4)-pyrimidina-4(1,2)-benzenacyclooctaphane

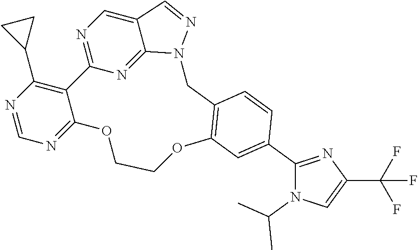 [SMILES: CC(C)n1cc(C(F)(F)F)nc1-c1ccc2c(c1)OCCOc1ncnc(C3CC3)c1-c1ncc3cnn(c3n1)C2]

To a mixture of the product of Step 4 (30 mg) and Ph₃P (30 mg) in DCM (2 mL) was added dropwise DIAD (26 mg) at room temperature under Ar atmosphere. The resulting mixture was stirred at rt for 1 h and then concentrated. The residue was purified by C18 flash column chromatography (grain size: 20-45 m, pore size distribution: 100 Å) eluted with water (0.1% formic acid)/CH₃CN (0 to 70% CH₃CN) to afford the title product as a white solid (10 mg). LCMS (ESI): 563.38 [M+1]⁺. ¹H NMR (500 MHz, CDCl₃) δ 9.27 (s, 1H), 8.73 (s, 1H), 8.17 (s, 1H), 7.83 (d, J=7.7 Hz, 1H), 7.42 (s, 1H), 7.11 (d, J=7.7 Hz, 1H), 7.06 (s, 1H), 5.83 (br, 2H), 4.71 (t, 2H), 4.63-4.52 (m, 1H), 4.30-4.13 (m, 2H), 2.80-2.68 (m, 1H), 1.45 (d, J=6.7 Hz, 6H), 1.38-1.33 (m, 2H), 1.13-1.04 (m, 2H).

### Example 3

### 1⁶-cyclopropyl-4⁴-(1-isopropyl-4-(trifluoromethyl)-1H-imidazol-2-yl)-2¹H-5,10-dioxa-2(6,1)-pyrazolo[3,4-d]pyrimidina-1(5,4)-pyrimidina-4(1,2)-benzenacyclodecaphane

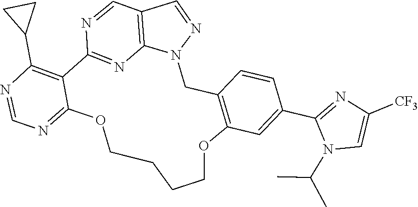 [SMILES: CC(C)n1cc(C(F)(F)F)nc1-c1ccc2c(c1)OCCCCOc1ncnc(C3CC3)c1-c1ncc3cnn(c3n1)C2]

### Step 1 methyl 4-formyl-2-methoxybenzoate

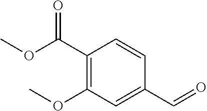

A mixture of 4-bromo-3-methoxybenzaldehyde (25.0 g), Pd(OAc)₂ (1.3 g) and 2-(dicyclohexylphosphino)-2′,4′,6′-tri-i-propyl-1,1′-biphenyl (3.4 g) in methanol (300 mL) was stirred at 70° C. overnight under CO atmosphere. The reaction mixture was cooled to r.t. and filtered through celite pad and washed with methanol (200 mL). The filtrate was concentrated under vacuum to afford the title product (27.0 g, crude) as a black oil.

### Step 2 methyl 2-methoxy-4-(4-(trifluoromethyl)-1H-imidazol-2-yl)benzoate

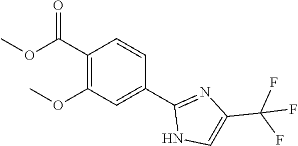 [SMILES: COC(=O)c1ccc(-c2nc(C(F)(F)F)c[nH]2)cc1OC]

To a mixture of 3,3-dibromo-1,1,1-trifluoropropan-2-one (52.0 g) in water (60 mL) was added KOAc (23.0 g) at r.t. After stirring at 90° C. for 1 h, a solution of the product of Step 1 (25.0 g) in methanol (200 mL) and NH₄OH (120 mL, 25-28% wt) were added to the reaction mixture at r.t. The mixture was stirred at 70° C. for 5 h and then cooled to r.t. and diluted with water (500 mL). The mixture was stirred at r.t. for 1 h and the solid was collected by filtration and washed with water. The solid was air-dried to afford the title product as yellow solid (27.0 g). LCMS (ESI): 301.35 [M+1]⁺.

### Step 3 methyl 4-(1-isopropyl-4-(trifluoromethyl)-1H-imidazol-2-yl)-2-methoxy benzoate

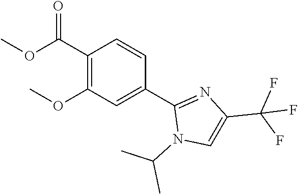 [SMILES: COC(=O)c1ccc(-c2nc(C(F)(F)F)cn2C(C)C)cc1OC]

A mixture of the product of Step 2 (27.0 g), 2-iodopropane (61.2 g) and Cs₂CO₃ (87.9 g) in DMF (150 mL) was stirred at 80° C. for 18 h. The reaction solution was poured into water (1.0 L) and extracted with EtOAc (500 mL×2). The combined organic layer was washed with brine (400 mL), dried over Na₂SO₄ and concentrated under vacuum. The residue was purified by SGCC eluted with EtOAc/PE (0 to 60% EtOAc) to afford the title product as a yellow solid (28.0 g). LCMS (ESI): 343.4 [M+1]⁺.

### Step 4 methyl 2-hydroxy-4-(1-isopropyl-4-(trifluoromethyl)-1H-imidazol-2-yl) benzoate

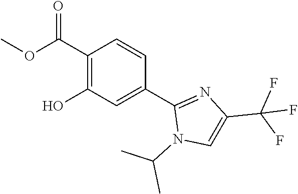 [SMILES: COC(=O)c1ccc(-c2nc(C(F)(F)F)cn2C(C)C)cc1O]

To a solution of the product of Step 3 (28.0 g) in DCM (150 mL) was added dropwise boron tribromide (163 mL, 1M, in DCM) at −78° C. over 10 min. The reaction solution was stirred for 1 h from −78° C. to r.t. The reaction was quenched by methanol (50 mL) at 0° C. and the reaction mixture was concentrated under vacuum. The residue was dissolved in methanol (150 mL) and then treated with thionyl chloride (10 mL) at 0° C. The resulting solution was stirred at 70° C. overnight and then concentrated under vacuum. The residue was dissolved EtOAc (500 mL) and washed with saturated NaHCO₃ (300 mL), brine (300 mL). The organic phase was dried over anhydrous Na₂SO₄ and concentrated under vacuum to afford the title product as a yellow solid (21.0 g). LCMS (ESI): 329.4 [M+1]⁺.

### Step 5 methyl 2-(benzyloxy)-4-(1-isopropyl-4-(trifluoromethyl)-1H-imidazol-2-yl) benzoate

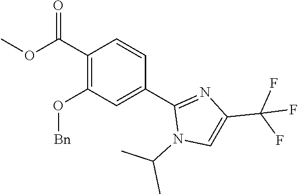 [SMILES: COC(=O)c1ccc(-c2nc(C(F)(F)F)cn2C(C)C)cc1OCc1ccccc1]

A mixture of the product of Step 4 (21.0 g), (chloromethyl)benzene (12.2 g) and Cs₂CO₃ (41.7 g) in DMF (150 mL) was stirred at 50° C. for 1 h. The reaction solution was poured into water (800 mL) and extracted with EtOAc (300 mL×2). The combined organic layer was washed with brine (300 mL), dried over Na₂SO₄ and concentrated under vacuum. The residue was purified using SGCC eluted with EtOAc/PE (0 to 80% EtOAc) to afford the title product as a white solid (24.0 g). LCMS (ESI): 419.5 [M+1]⁺.

### Step 6 (2-(benzyloxy)-4-(1-isopropyl-4-(trifluoromethyl)-1H-imidazol-2-yl)phenyl) methanol

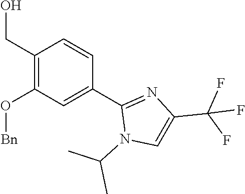 [SMILES: CC(C)n1cc(C(F)(F)F)nc1-c1ccc(CO)c(OCc2ccccc2)c1]

To a solution of the product of Step 5 (24.0 g) in THE (160 mL) was added dropwise DIBAL-H (143 mL, 1M in hexane) at 0° C. The reaction solution was stirred at 0° C. for 1 h. The reaction solution was quenched by Na₂SO₄ decahydrate (50 g) and then stirred at r.t. for 30 min. The resulting mixture was filtered through a celite pad and washed with methanol and DCM (1/5, 1 L). The filtrate was concentrated under vacuum to afford the title product as a yellow solid (20.0 g). LCMS (ESI): 391.5 [M+1]⁺.

### Step 7 6-chloro-1-((2-(trimethylsilyl)ethoxy)methyl)-1H-pyrazolo[3,4-d]pyrimidine

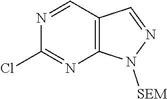 [SMILES: C[Si](C)(C)CCOCn1ncc2cnc(Cl)nc21]

To a mixture of 6-chloro-1H-pyrazolo[3,4-d]pyrimidine (25.0 g) and Cs₂CO₃ (30.0 g) in DMF (300 mL), (2-(chloromethoxy)ethyl)trimethylsilane was added slowly. The reaction mixture was at r.t. for 18 h and then poured into 200 mL EtOAc and washed with 3×100 mL NaHCO₃ (aq.) and 100 mL saturated brine. The organic phase was dried over anhydrous Na₂SO₄ and concentrated. The residue was purified by SGCC (eluted with PE/EtOAc: from 100:0 to 10:1) to afford the title compound as a yellow solid (40.0 g). LCMS (ESI): 285.6 [M+1]⁺.

### Step 8 6-(4-cyclopropyl-6-methoxypyrimidin-5-yl)-1-((2-(trimethylsilyl)ethoxy)methyl)-1H-pyrazolo [3,4-d]pyrimidine

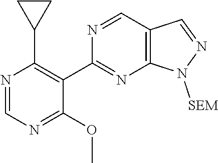 [SMILES: COc1ncnc(C2CC2)c1-c1ncc2cnn(COCC[Si](C)(C)C)c2n1]

To a mixture of the product of Step 7 (5.0 g) and 4-cyclopropyl-6-methoxy-5-(4,4,5,5-tetramethyl-1,3,2-dioxaborolan-2-yl)pyrimidine (6.8 g) in 1,2-dioxane/water (20/2 mL) at r.t. was added Pd₂(dba)₃ (1.6 g), 2-(dicyclohexylphosphino)-2,4,6-triisopropylbiphenyl (1.68 g) and CsF (5.3 g). The reaction mixture was then stirred at 100° C. for 18 h and then poured into 100 mL ice-water. The mixture was extracted with EtOAc (3×50 mL). The combined organic layer was washed with saturated brine (50 mL), dried over anhydrous Na₂SO₄ and concentrated. The residue was purified by SGCC (eluted with PE/EtOAc: from 100:0 to 10:1) to afford the title compound as a yellow solid (6.0 g). LCMS (ESI): 399.2 [M+1]⁺.

### Step 9 6-(4-cyclopropyl-6-methoxypyrimidin-5-yl)-1H-pyrazolo[3,4-d]pyrimidine

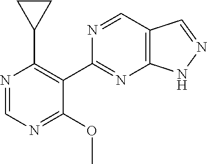 [SMILES: COc1ncnc(C2CC2)c1-c1ncc2cn[nH]c2n1]

A mixture of the product of step 8 (2.7 g) in TFA (10 ml) was stirred at 80° C. for 2 h. The reaction mixture was then concentrated. The residue was treated with ammonia (10.0 ml, 7.0M solution in methanol) and stirred at 25° C. for 2 h. The reaction mixture was concentrated and the residue was purified by SGCC (eluted with PE/EtOAc: from 100:0 to 5:1) to afford the title compound as a yellow solid (2.0 g). LCMS (ESI): 269.2 [M+1]⁺.

### Step 10 1-(2-(benzyloxy)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

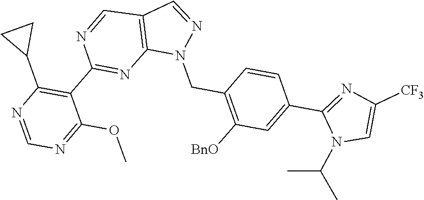

To a solution of the product of Step 6 (3.8 g), the product of Step 9 (2.0 g) and tributylphosphine (3.1 g) in THE (40 mL) was added dropwise DIAD (3.1 g) at r.t. The resulting mixture was stirred at r.t. for 2 h and then concentrated. The residue was purified by SGCC eluted with EtOAc and PE (0 to 80% EtOAc) to afford the title product (3.8 g) as a yellow solid. LCMS (ESI): 641.6 [M+1]⁺.

### Step 11 2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenol

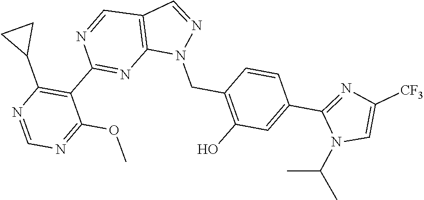

A solution of the product of Step 10 (3.0 g) in TFA (50 mL) was stirred at 100° C. overnight. The reaction solution was concentrated and the residue was diluted with EtOAc (60 mL). The solution was washed with saturated NaHCO₃ (30 mL), brine (30 mL), dried over anhydrous Na₂SO₄ and concentrated. The residue was purified by SGCC eluted with EtOAc/PE (0 to 100% EtOAc) to afford the title product as a white solid (2.0 g). LCMS (ESI): 551.6 [M+1]⁺.

### Step 12 1-(2-(4-((tert-butyldimethylsilyl)oxy)butoxy)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

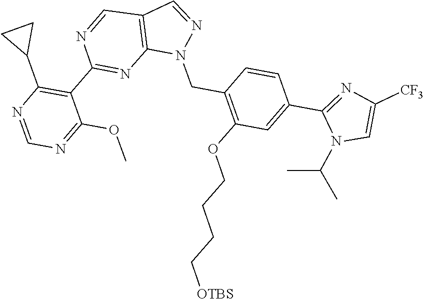

To a solution of the product of Step 11 (200 mg), 4-((tert-butyldimethylsilyl)oxy)butan-1-ol (111 mg) and Ph₃P (191 mg) in THE (3 mL) was added dropwise DIAD (147 mg) at r.t. After stirring at r.t. for 1 h, the reaction solution was concentrated and the residue was purified by C18 flash column chromatography eluted with acetonitrile/water (100% to 30% water, 0.3% formic acid in water) to afford the title product (180 mg) as a white solid. LCMS (ESI): 737.9 [M+1]⁺.

### Step 13 6-cyclopropyl-5-(1-(2-(4-hydroxybutoxy)-4-(1-isopropyl-4-(trifluoromethyl)-1H-imidazol-2-yl)benzyl)-1H-pyrazolo[3,4-d]pyrimidin-6-yl)pyrimidin-4-ol

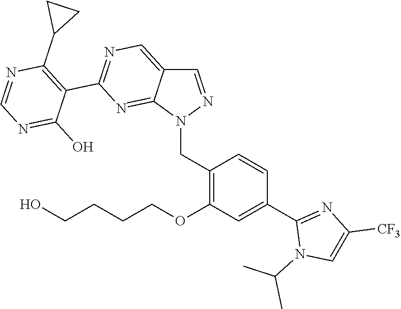 [SMILES: CC(C)n1cc(C(F)(F)F)nc1-c1ccc(Cn2ncc3cnc(-c4c(O)ncnc4C4CC4)nc32)c(OCCCCO)c1]

To a solution of the product of Step 12 (180 mg) in 1,4-dioxane (2 mL) was added HCl (3M in water, 2 mL) at r.t. After stirring at 80° C. for 1 h, the reaction mixture was cooled to r.t. and diluted by EtOAc (20 mL). The solution was washed with saturated NaHCO₃ (20 mL), brine (20 mL). The organic layer was dried over anhydrous Na₂SO₄ and then concentrated to afford the title product (120 mg) as a yellow solid. LCMS (ESI): 609.6 [M+1]⁺.

### Step 14 1⁶-cyclopropyl-4⁴-(1-isopropyl-4-(trifluoromethyl)-1H-imidazol-2-yl)-2¹H-5,10-dioxa-2(6,1)-pyrazolo[3,4-d]pyrimidina-1(5,4)-pyrimidina-4(1,2)-benzenacyclodecaphane

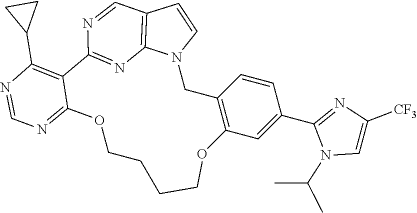 [SMILES: CC(C)n1cc(C(F)(F)F)nc1-c1ccc2c(c1)OCCCCOc1ncnc(C3CC3)c1-c1ncc3ccn(c3n1)C2]

To a solution of the product of Step 13 (90 mg) and Ph₃P (116 mg) in DCM (5 mL) was added dropwise DIAD (90 mg) at r.t. The resulting mixture was stirred at r.t. for 1 h and was then concentrated. The residue was purified using C18 flash column chromatography eluted with acetonitrile/water (100% to 30% water, 0.1% formic acid in water) to afford the title product (7 mg) as a white solid. LCMS (ESI): 591.55 [M+1]⁺.

### Example 4

### 1⁶-cyclopropyl-4⁴-(1-isopropyl-4-(trifluoromethyl)-1H-imidazol-2-yl)-2¹H-7-oxa-2(6,1)-pyrazolo[3,4-d]pyrimidina-1(5,4)-pyrimidina-4(1,2)-benzenacycloheptaphane

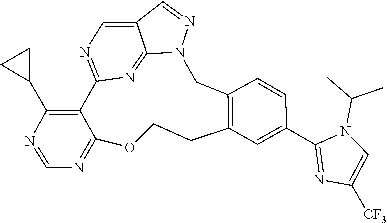 [SMILES: CC(C)n1cc(C(F)(F)F)nc1-c1ccc2c(c1)CCOc1ncnc(C3CC3)c1-c1ncc3cnn(c3n1)C2]

### Step 1 2-hydroxy-4-(4,4,5,5-tetramethyl-1,3,2-dioxaborolan-2-yl)benzaldehyde

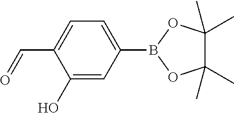 [SMILES: CC1(C)OB(c2ccc(C=O)c(O)c2)OC1(C)C]

A mixture of 4-bromo-2-hydroxybenzaldehyde (10.0 g), 4,4,4′,4′,5,5,5′,5′-octamethyl-2,2′-bi(1,3,2-dioxaborolane) (25.4 g), KOAc (20.0 g), (dppf)PdCl₂ (1.1 g) in 1,4-dioxane (300 mL) was stirred at 90° C. overnight under Ar. The reaction solution was poured into water and extracted with EtOAc (3×150 mL). The combined organic layer was washed with water (100 mL), dried over Na₂SO₄ and concentrated. The residue was purified by SGCC (eluted with PE/EtOAc: from 100:0 to 5:1) to afford the title product as a pale yellow solid (11.0 g). LCMS (ESI): 249.2 [M+1]⁺.

### Step 2 2-hydroxy-4-(1-isopropyl-4-(trifluoromethyl)-1H-imidazol-2-yl)benzaldehyde

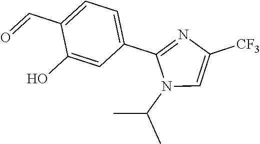 [SMILES: CC(C)n1cc(C(F)(F)F)nc1-c1ccc(C=O)c(O)c1]

A mixture of the product of Step 1 (2.0 g), 2-bromo-1-isopropyl-4-(trifluoromethyl)-1H-imidazole (2.48 g), [2′-(amino-κN)[1,1′-biphenyl]-2-yl-κC]chloro[dicyclohexyl[2′,4′,6′-tris(1-methylethyl)[1,1′-biphenyl]-2-yl]phosphine]palladium (315 mg), 2-(dicyclohexylphosphino)-2′,4′,6′-tri-i-propyl-1,1′-biphenyl (382 mg) and potassium phosphate (3.4 g) in 1,4-dioxane/water (50 mL/5 mL) was stirred for 16 h at 90° C. under Ar. The reaction mixture was concentrated and purified by SGCC (eluted with PE/EtOAc: from 100:0 to 4:1) to afford the title compound as a yellow solid (2.2 g). LCMS (ESI): 299.2 [M+1]⁺.

### Step 3 2-formyl-5-(1-isopropyl-4-(trifluoromethyl)-1H-imidazol-2-yl)phenyltrifluoromethane-sulfonate

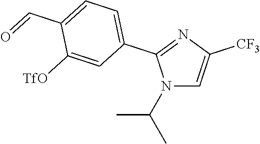 [SMILES: CC(C)n1cc(C(F)(F)F)nc1-c1ccc(C=O)c(OS(=O)(=O)C(F)(F)F)c1]

To a solution of the product of Step 2 (1.2 g), pyridine (0.95 g) in DCM (20 mL) was added trifluoromethanesulfonic anhydride (1.69 g) at 0° C. The mixture was warmed to r.t. over 2 h and then treated with ammonium chloride (1N, 30 mL) and extracted with DCM (3×50 mL). The organic layer was concentrated in vacuum. The residue was purified by SGCC (eluted with PE:EtOAc=5:1) to give the product as a yellow oil (1.4 g). LCMS (ESI): 431.3 [M+1]⁻.

### Step 4 (E)-4-(1-isopropyl-4-(trifluoromethyl)-1H-imidazol-2-yl)-2-(2-methoxyvinyl)benzaldehyde

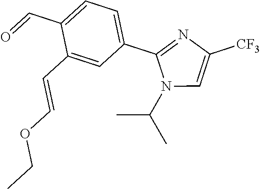 [SMILES: CCO/C=C/c1cc(-c2nc(C(F)(F)F)cn2C(C)C)ccc1C=O]

A mixture of the product of Step 3 (620 mg), (E)-2-(2-ethoxyvinyl)-4,4,5,5-tetramethyl-1,3,2-dioxaborolane (860 mg), Pd₂(dba)₃ (132 mg), tricyclohexyl phosphine (82 mg) and K₂CO₃ (400 mg) in 1,4-dioxane/water (20 mL/1 mL) was stirred for 16 h at 90° C. under Ar. The reaction mixture was concentrated and the residue purified by SGCC (eluted with PE/EtOAc: from 100:0 to 3:1) to afford the title compound as a yellow solid (400 mg). LCMS (ESI): 353.3 [M+1]⁺.

### Step 5 (E)-(4-(1-isopropyl-4-(trifluoromethyl)-1H-imidazol-2-yl)-2-(2-methoxyvinyl)phenyl)methanol

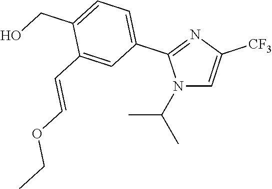 [SMILES: CCO/C=C/c1cc(-c2nc(C(F)(F)F)cn2C(C)C)ccc1CO]

To a solution of the product of Step 4 (400 mg) in methanol (10 mL) cooled at 0° C. was added NaBH₄ (50 mg). The mixture was then stirred at r.t. for 2 h. The mixture was then treated with ammonium chloride (1N, 30 mL) and extracted with DCM (3×20 mL). The organic layer was concentrated in vacuum. The residue was purified by SGCC (eluted with PE/EtOAc: from 100:0 to 3:1) to give the product as a yellow solid (380 mg). LCMS (ESI): 355.3 [M+1]⁺.

### Step 6 (E)-6-chloro-1-(2-(2-ethoxyvinyl)-4-(1-isopropyl-4-(trifluoromethyl)-1H-imidazol-2-yl)benzyl)-1H-pyrazolo[3,4-d]pyrimidine

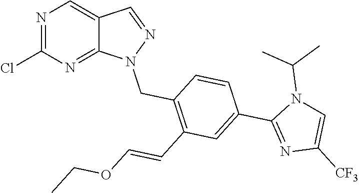 [SMILES: CCO/C=C/c1cc(-c2nc(C(F)(F)F)cn2C(C)C)ccc1Cn1ncc2cnc(Cl)nc21]

To a solution of the product of Step 5 (360 mg), 6-chloro-1H-pyrazolo[3,4-d]pyrimidine (155 mg) and n-Bu₃P (303 mg) in THE (10 mL) cooled to 0° C. was added DIAD (250 mg in 1 mL THF) over 5 min. The mixture reaction was then stirred at r.t. for 4 h. The reaction mixture was then concentrated and purified by SGCC (eluted with PE/EtOAc: from 100:0 to 1:1) to afford the title compound as a white solid (420 mg). LCMS (ESI): 491.3 [M+1]⁺.

### Step 7 (E)-6-(4-cyclopropyl-6-methoxypyrimidin-5-yl)-1-(2-(2-ethoxyvinyl)-4-(1-isopropyl-4-(trifluoromethyl)-1H-imidazol-2-yl)benzyl)-1H-pyrazolo[3,4-d]pyrimidine

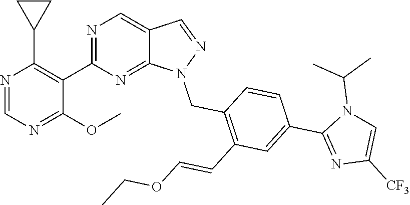 [SMILES: CCO/C=C/c1cc(-c2nc(C(F)(F)F)cn2C(C)C)ccc1Cn1ncc2cnc(-c3c(OC)ncnc3C3CC3)nc21]

To a mixture of the product of Step 6 (400 mg) and (4-cyclopropyl-6-methoxypyrimidin-5-yl)boronic acid (200 mg) in 1,4-dioxane/water (10/1 mL) at r.t. was added Pd₂(dba)₃ (60 mg) and 2-(dicyclohexylphosphino)-2,4,6-triisopropylbiphenyl (70 mg) and CsF (450 mg). The reaction mixture was stirred at 90° C. for 5 h and then poured into 30 mL ice water and extracted with EtOAc (3×50 mL). The combined organic layer was washed with saturated brine (50 mL), dried over anhydrous Na₂SO₄ and concentrated. The residue was purified by SGCC (eluted with PE/EtOAc: from 100:0 to 2:1) to afford the title compound as a pale yellow solid (420 mg). LCMS (ESI): 605.4 [M+1]⁺.

### Step 8 2-(2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl)acetaldehyde

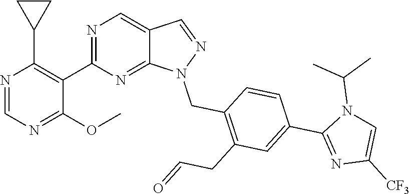 [SMILES: COc1ncnc(C2CC2)c1-c1ncc2cnn(Cc3ccc(-c4nc(C(F)(F)F)cn4C(C)C)cc3CC=O)c2n1]

A mixture of the product of Step 7 (400 mg) in a mixture of 1,4-dioxane/6 M HCl (10 mL/1 mL) was stirred for 30 min at rt. The mixture was then treated with NaHCO₃ (2N, 30 mL) and extracted with EtOAc (3×20 mL). The organic layer was dried over anhydrous Na₂SO₄ and concentrated in vacuum to afford the title compound as a white solid (350 mg). LCMS (ESI): 577.4 [M+1]⁺.

### Step 9 2-(2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl)ethan-1-ol

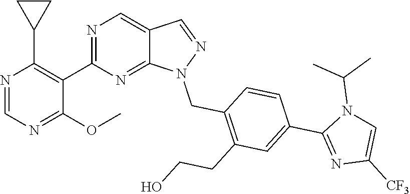 [SMILES: COc1ncnc(C2CC2)c1-c1ncc2cnn(Cc3ccc(-c4nc(C(F)(F)F)cn4C(C)C)cc3CCO)c2n1]

To a solution of the product of step 8 (300 mg) in THE (20 mL) was added dropwise a solution of DIBAL-H (0.8 mL, 1M in n-hexane) at 0° C. for 30 min. The reaction mixture was stirred at 0° C. for 1 h and then was stirred at r.t. for 3 h. The reaction was then quenched with aq. HCl (3M, 2 mL) and 20 mL water at 0° C. The mixture was extracted with EtOAc (3×30 mL). The combined organic layer was dried over anhydrous Na₂SO₄ and concentrated under vacuum to afford the title compound as a white solid (300 mg). LCMS (ESI): 579.4 [M+1]⁺.

### Step 10 1⁶-cyclopropyl-4⁴-(1-isopropyl-4-(trifluoromethyl)-1H-imidazol-2-yl)-2¹H-7-oxa-2(6,1)-pyrazolo[3,4-d]pyrimidina-1(5,4)-pyrimidina-4(1,2)-benzenacycloheptaphane

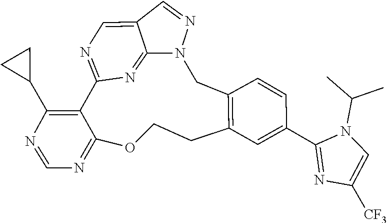 [SMILES: CC(C)n1cc(C(F)(F)F)nc1-c1ccc2c(c1)CCOc1ncnc(C3CC3)c1-c1ncc3cnn(c3n1)C2]

Starting with the product of Step 9, the title product was obtained as a white solid by following the same procedures as described in Step 13 and Step 14 of Example 3. LCMS (ESI): 547.6 [M+1]⁺.

### Example 5

### 1⁶-cyclopropyl-4⁴-(1-isopropyl-4-(trifluoromethyl)-1H-imidazol-2-yl)-2¹H-5,9-dioxa-2(6,1)-pyrazolo[3,4-d]pyrimidina-1(5,4)-pyrimidina-4(1,2)-benzenacyclononaphane

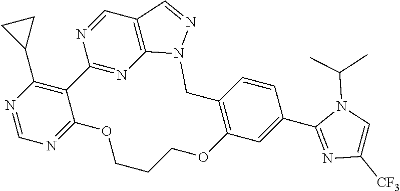 [SMILES: CC(C)n1cc(C(F)(F)F)nc1-c1ccc2c(c1)OCCCOc1ncnc(C3CC3)c1-c1ncc3cnn(c3n1)C2]

Using 3-(tert-butyldimethylsilyloxy) propanol as the starting material in Step 12 of Example 3, the title compound was obtained following the same procedures as described for Example 3. LCMS (ESI): 577.2 [M+1]⁺. ¹H NMR (500 MHz, CDCl₃) δ 9.29 (s, 1H), 8.69 (s, 1H), 8.19 (s, 1H), 7.86 (d, 1H), 7.43 (d, 1H), 7.12 (dd, 1H), 7.10 (s, 1H), 6.03-5.64 (brs, 2H), 4.91-4.60 (brs, 2H), 4.60-4.54 (m, 1H), 4.32 (t, J=4.8 Hz, 2H), 2.37-2.30 (m, 1H), 2.27-2.18 (m, 2H), 1.45 (d, 6H), 1.34-1.30 (m, 2H), 1.08-0.98 (m, 2H).

### Example 6

### 1⁶-chloro-4⁴-(1-isopropyl-4-(trifluoromethyl)-1H-imidazol-2-yl)-2¹H-5,8-dioxa-2(6,1)-pyrazolo[3,4-d]pyrimidina-1,4(1,2)-dibenzenacyclooctaphane

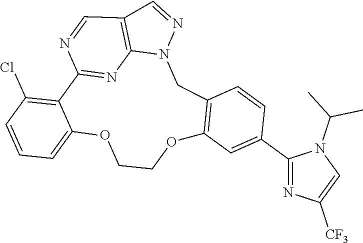 [SMILES: CC(C)n1cc(C(F)(F)F)nc1-c1ccc2c(c1)OCCOc1cccc(Cl)c1-c1ncc3cnn(c3n1)C2]

### Step 1 2-(2-((tert-butyldimethylsilyl)oxy)ethoxy)-4-(1-isopropyl-4-(trifluoromethyl)-1H-imidazol-2-yl)benzaldehyde

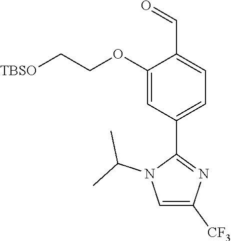 [SMILES: CC(C)n1cc(C(F)(F)F)nc1-c1ccc(C=O)c(OCCO[Si](C)(C)C(C)(C)C)c1]

A mixture of the product of Step 2 in Example 3 (0.9 g), (2-bromoethoxy)(tert-butyl)dimethylsilane (1.4 g) and Cs₂CO₃ (1.9 g) in DMF (20 mL) was stirred for 3 h at 60° C. The mixture was then treated with ammonium chloride (1N, 100 mL) and extracted with EtOAc (3×50 mL). The combined organic layer was then dried over anhydrous Na₂SO₄, concentrated and the residue was purified by SGCC (eluted with PE/EtOAc: from 100:0 to 2:1) to afford the title compound as yellow oil (1.3 g). LCMS (ESI): 457.3 [M+1]⁺.

### Step 2 (2-(2-((tert-butyldimethylsilyl)oxy)ethoxy)-4-(1-isopropyl-4-(trifluoromethyl)-1H-imidazol-2-yl)phenyl)methanol

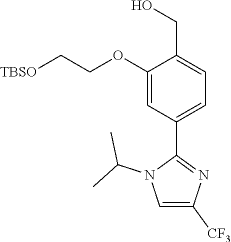 [SMILES: CC(C)n1cc(C(F)(F)F)nc1-c1ccc(CO)c(OCCO[Si](C)(C)C(C)(C)C)c1]

Starting with the product of Step 1 (1.0 g), the title product (1.0 g) was obtained as yellow oil by following the same procedure as described in Step 5 of Example 4. LCMS (ESI): 459.4 [M+1]⁺.

### Step 3 1-(2-(2-((tert-butyldimethylsilyl)oxy)ethoxy)-4-(1-isopropyl-4-(trifluoromethyl)-1H-imidazol-2-yl)benzyl)-6-chloro-1H-pyrazolo[3,4-d]pyrimidine

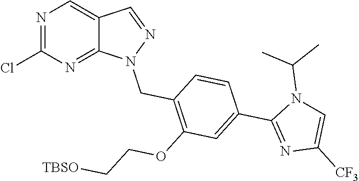 [SMILES: CC(C)n1cc(C(F)(F)F)nc1-c1ccc(Cn2ncc3cnc(Cl)nc32)c(OCCO[Si](C)(C)C(C)(C)C)c1]

To a solution of the product of Step 2 (1.0 g), 6-chloro-1H-pyrazolo[3,4-d]pyrimidine (300 mg) and tributyl phosphine (509 mg) in THF (20 mL) at 0° C., was added DIAD (441 mg in 1 mL THF) over 5 min. The mixture reaction was stirred at r.t. for 4 h. The reaction mixture was then concentrated and purified by SGCC (eluted with PE/EtOAc: from 100:0 to 1:1) to afford the title compound as a yellow solid (900 mg). LCMS (ESI): 595.4 [M+1]⁺.

### Step 4 1-(2-(2-((tert-butyldimethylsilyl)oxy)ethoxy)-4-(1-isopropyl-4-(trifluoromethyl)-1H-imidazol-2-yl)benzyl)-6-(2-chloro-6-methoxyphenyl)-1H-pyrazolo[3,4-d]pyrimidine

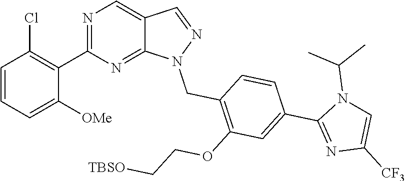 [SMILES: COc1cccc(Cl)c1-c1ncc2cnn(Cc3ccc(-c4nc(C(F)(F)F)cn4C(C)C)cc3OCCO[Si](C)(C)C(C)(C)C)c2n1]

To a mixture of the product of Step 3 (600 mg) and (2-chloro-6-methoxyphenyl)boronic acid (500 mg) in 1,4-dioxane/water (20/2 mL) at r.t. was added Pd₂(dba)₃ (90 mg) and 2-(dicyclohexylphosphino)-2,4,6-triisopropylbiphenyl (94 mg) and CsF (450 mg). After the reaction mixture was stirred at 90° C. for 4 h, the reaction mixture was then poured into 30 mL ice-water, and extracted with EtOAc (3×50 mL). The combined organic layer was washed with 50 mL saturated brine, dried over anhydrous Na₂SO₄ and concentrated. The residue was purified by SGCC (eluted with PE/EtOAc: from 100:0 to 2:1) to afford the title compound as pale yellow solid (500 mg). LCMS (ESI): 701.4 [M+1]⁺.

### Step 5 3-chloro-2-(1-(2-(2-hydroxyethoxy)-4-(1-isopropyl-4-(trifluoromethyl)-1H-imidazol-2-yl) benzyl)-1H-pyrazolo[3,4-d]pyrimidin-6-yl)phenol

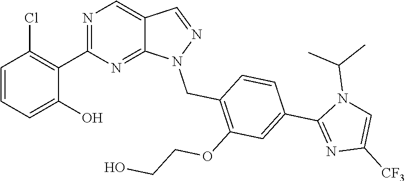 [SMILES: CC(C)n1cc(C(F)(F)F)nc1-c1ccc(Cn2ncc3cnc(-c4c(O)cccc4Cl)nc32)c(OCCO)c1]

To a solution of the product of Step 4 (0.42 g) in DCM (10 mL) was added boron tribromide (0.6 mL, 1 M in DCM) at −78° C. After the reaction mixture was stirred at r.t. for 3 h, the reaction mixture was then treated with methanol (1.0 mL) and NaHCO₃ (2N, 60 mL). The mixture was extracted with EtOAc (3×20 mL). The combined organic layer was dried over anhydrous Na₂SO₄ and concentrated in vacuum to afford the title compound as white solid (300 mg). LCMS (ESI): 573.3 [M+1]⁺.

### Step 6 1⁶-chloro-4⁴-(1-isopropyl-4-(trifluoromethyl)-1H-imidazol-2-yl)-2¹H-5,8-dioxa-2(6,1)-pyrazolo [3,4-d]pyrimidina-1,4(1,2)-dibenzenacyclooctaphane

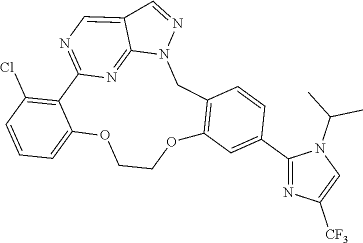 [SMILES: CC(C)n1cc(C(F)(F)F)nc1-c1ccc2c(c1)OCCOc1cccc(Cl)c1-c1ncc3cnn(c3n1)C2]

Starting with the product of Step 5, the title product was obtained as a white solid by following the same procedures as described in Step 14 of Example 3. LCMS (ESI): 555.4 [M+1]⁺. ¹H NMR (500 MHz, CDCl₃) δ 9.30 (s, 1H), 8.16 (s, 1H), 7.78 (d, 1H), 7.45-7.38 (m, 2H), 7.31 (s, 1H), 7.17 (d, 1H), 7.09 (d, 1H), 7.00 (s, 1H), 5.37 (brs, 2H), 4.63-4.54 (m, 1H), 4.53 (s, 2H), 4.18 (s, 2H), 1.45 (d, 6H).

### Example 7

### 1⁶-isopropyl-4⁴-(1-isopropyl-4-(trifluoromethyl)-1H-imidazol-2-yl)-2¹H-5,8-dioxa-2(6,1)-pyrazolo[3,4-d]pyrimidina-1,4(1,2)-dibenzenacyclooctaphane

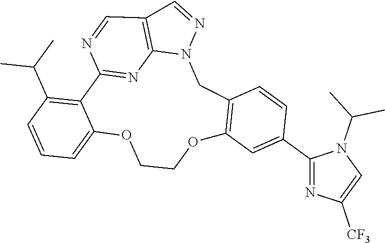 [SMILES: CC(C)c1cccc2c1-c1ncc3cnn(c3n1)Cc1ccc(-c3nc(C(F)(F)F)cn3C(C)C)cc1OCCO2]

To a mixture of Example 5 (200 mg), lithium chloride (76 mg), isopropylzinc bromide (5 mL, 0.5 M in THF) in THE (10 mL) were added Pd(OAc)₂ (40 mg) and 2-dicyclohexylphosphino-2′,6′-bis(dimethylamino)-1,1′-bipheny (78 mg) at 0° C. The reaction mixture was stirred 12 h at r.t. and then quenched with a saturated ammonium chloride solution (10 mL), extracted with EtOAc (3×15). The combined organic layer was dried over anhydrous Na₂SO₄ and concentrated. The residue was purified by SGCC (eluted with PE/EtOAc: from 100:0 to 2:1) to afford the title compound as yellow solid (35 mg). LCMS (ESI): 563.5 [M+1]⁺.

### Example 8

### 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,2)-benzenacyclononaphane

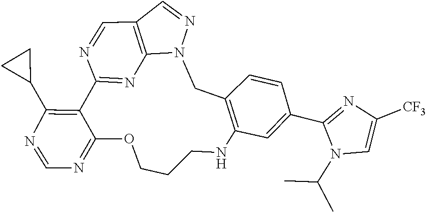 [SMILES: CC(C)n1cc(C(F)(F)F)nc1-c1ccc2c(c1)NCCCOc1ncnc(C3CC3)c1-c1ncc3cnn(c3n1)C2]

### Step 1 1-isopropyl-4-(trifluoromethyl)-1H-imidazole

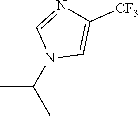

A mixture of 4-(trifluoromethyl)-1H-imidazole (30 g), 2-iodopropane (45 g) in DMF (600 mL) was added Cs₂CO₃ (144 g) and stirred at r.t. for 18 h. The mixture was diluted with EtOAc (1.8 L) and washed with brine (6×1 L), the organic layer was concentrated and the residue was purified by SGCC (eluted with PE/EtOAc: from 100:0 to 10:1) to obtain the product as brown oil (36.5 g). LCMS (ESI): 179.3 [M+1]⁺.

### Step 2 2-bromo-1-isopropyl-4-(trifluoromethyl)-1H-imidazole

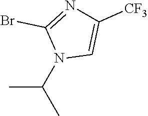

To a solution of the product of Step 1 (36.5 g) in THE (400 mL) at −78° C. under Ar was added n-butyllithium (2.5 M in hexane, 107 mL) dropwise. The resulting mixture was stirred for additional 0.5 h and then treated with a solution of CBr₄ (109 g) in THE (100 mL). The reaction mixture was stirred at r.t. for 1 h and quenched with aqueous ammonium chloride (1 L) solution and extracted with EtOAc (3×500 mL). The organic layer was concentrated and the residue was purified by SGCC (eluted with PE/EtOAc: from 100:0 to 10:1) to afford the title compound as brown oil (27.6 g). LCMS (ESI): 257.2 [M+1]⁺.

### Step 3 (4-(1-isopropyl-4-(trifluoromethyl)-1H-imidazol-2-yl)-2-nitrophenyl)methanol

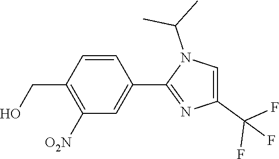 [SMILES: CC(C)n1cc(C(F)(F)F)nc1-c1ccc(CO)c([N+](=O)[O-])c1]

To a solution of (4-bromo-2-nitrophenyl)methanol (20 g), bis (pinacolato) diboron (24 g), KOAc (25.5 g) in 1,4-dioxane (300 mL) was added Pd(dppf)Cl₂ (6.3 g) and the mixture was stirred at 100° C. for 1 h under Ar. After being cooled to r.t., chloro(2-dicyclohexylphosphino-2′,4′,6′-triisopropyl-1,1′-biphenyl)[2-(2′-amino-1,1′-biphenyl)]palladium(II) (6.8 g), the product of Step 2 (33.3 g), potassium phosphate tribasic (55.1 g) and 30 mL water were added to the reaction mixture. After stirring at 100° C. for 3 h under Ar, the reaction mixture was concentrated and the residue was purified by SGCC (eluted with PE/EtOAc: from 100:0 to 1:1) to obtain the product as brown oil (43 g). LCMS (ESI): 330.3 [M+1]⁺.

### Step 4 2-(4-(chloromethyl)-3-nitrophenyl)-1-isopropyl-4-(trifluoromethyl)-1H-imidazole

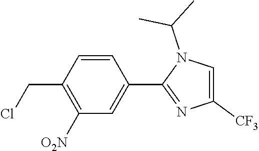 [SMILES: CC(C)n1cc(C(F)(F)F)nc1-c1ccc(CCl)c([N+](=O)[O-])c1]

To a solution of the product of Step 3 (43 g) in DCM (300 mL) was added thionyl chloride (50 g). The reaction mixture was stirred at r.t. for 2 h. The reaction mixture was then concentrated and diluted with EtOAc (500 mL). The organic phase was neutralized with aqueous potassium bicarbonate and washed with brine (300 mL). The organic layer was concentrated to obtain the crude product as brown oil (42 g). LCMS (ESI): 348.2 [M+1]⁺.

### Step 5 6-(4-cyclopropyl-6-methoxypyrimidin-5-yl)-1-(4-(1-isopropyl-4-(trifluoromethyl)-1H-imidazol-2-yl)-2-nitrobenzyl)-1H-pyrazolo[3,4-d]pyrimidine

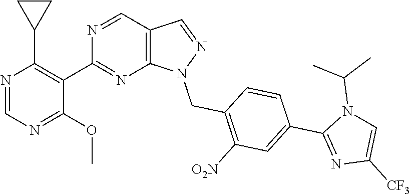 [SMILES: COc1ncnc(C2CC2)c1-c1ncc2cnn(Cc3ccc(-c4nc(C(F)(F)F)cn4C(C)C)cc3[N+](=O)[O-])c2n1]

To a mixture of 6-(4-cyclopropyl-6-methoxypyrimidin-5-yl)-1H-pyrazolo[3,4-d]pyrimidine (2.0 g), K₂CO₃ (3.1 g) in DMF (30 mL) at 0° C., was added the product of Step 4 (3.8 g) in 10 mL DMF. The reaction mixture was stirred at r.t. for 1 h and was then concentrated. The residue was purified by SGCC (eluted with PE/EtOAc: from 100:0 to 2:3) to afford the title product as brown solid (2.0 g). LCMS (ESI): 580.3 [M+1]⁺.

### Step 6 2-((6-(4-cyclopropyl-6-methoxypyrimidin-5-yl)-1H-pyrazolo[3,4-d]pyrimidin-1-yl)methyl)-5-(1-isopropyl-4-trifluoromethyl)-1H-imidazol-2-yl aniline

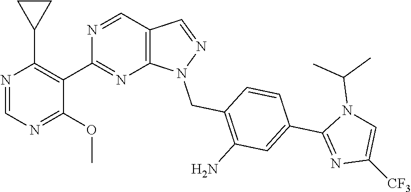 [SMILES: COc1ncnc(C2CC2)c1-c1ncc2cnn(Cc3ccc(-c4nc(C(F)(F)F)cn4C(C)C)cc3N)c2n1]

To a solution of the product of Step 5 (1.0 g), Zn powder (1.1 g) in methanol (15 mL) was added ammonium chloride (0.9 g). The reaction mixture was stirred at r.t. for 18 h and then filtered. The filtrate was concentrated and the residue was purified by SGCC (eluted with PE/EtOAc: from 100:0 to 1:1) to afford the product as brown solid (0.8 g). LCMS (ESI): 550.1 [M+1]⁺.

### Step 7 N-(3-((tert-butyldimethylsilyl)oxy)propyl)-2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)aniline

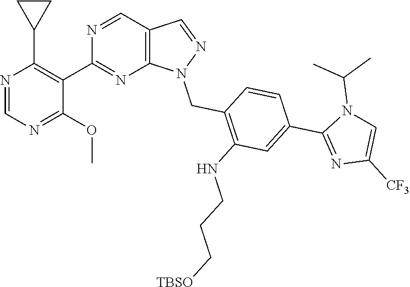 [SMILES: COc1ncnc(C2CC2)c1-c1ncc2cnn(Cc3ccc(-c4nc(C(F)(F)F)cn4C(C)C)cc3NCCCO[Si](C)(C)C(C)(C)C)c2n1]

To a solution of the product of Step 6 (0.4 g) and 3-((tert-butyldimethylsilyl)oxy)propanal (0.3 g) in DCM (4 mL) was added NaBH(OAc)₃ (0.8 g). The reaction mixture was stirred at r.t. for 1 h and then concentrated. The residue was purified by SGCC (eluted with DCM/methanol: from 100:0 to 20:1) to afford the product as brown oil (0.5 g). LCMS (ESI): 722.3 [M+1]⁺.

### Step 8 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,2)-benzenacyclononaphane

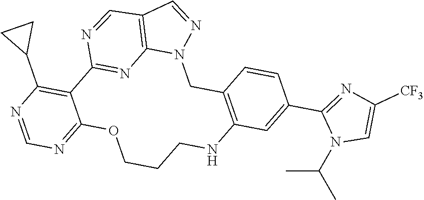 [SMILES: CC(C)n1cc(C(F)(F)F)nc1-c1ccc2c(c1)NCCCOc1ncnc(C3CC3)c1-c1ncc3cnn(c3n1)C2]

Starting with the product of Step 7, the title product was obtained as a white solid by following the same procedures as described in Step 13 and Step 14 of Example 3. LCMS (ESI): 576.5 [M+1]⁺.

### Example 9

### 1⁶-cyclopropyl-4⁴-(1-isopropyl-4-(trifluoromethyl)-1H-imidazol-2-yl)-2¹H-8-oxa-2(6,1)-pyrazolo[3,4-d]pyrimidina-1(5,4)-pyrimidina-4(1,2)-benzenacyclooctaphane

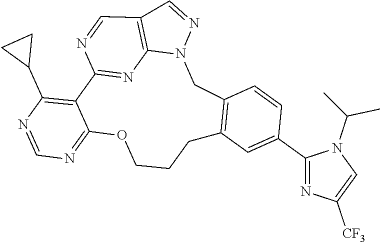 [SMILES: CC(C)n1cc(C(F)(F)F)nc1-c1ccc2c(c1)CCCOc1ncnc(C3CC3)c1-c1ncc3cnn(c3n1)C2]

### Step 1 methyl 2-bromo-4-(4-(trifluoromethyl)-1H-imidazol-2-yl)benzoate

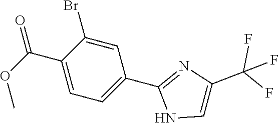 [SMILES: COC(=O)c1ccc(-c2nc(C(F)(F)F)c[nH]2)cc1Br]

To a mixture of 3,3-dibromo-1,1,1-trifluoropropan-2-one (17.8 g) in water (20 mL) was added KOAc (7.3 g) at r.t. The mixture was stirred at 90° C. for 1 h and cooled to 0° C., followed by addition of a solution of methyl 2-bromo-4-formylbenzoate (10.0 g) in methanol (100 mL) and aqueous ammonia (26 mL, 25% wt). The resulting mixture was stirred at 70° C. for 5 h and then concentrated. The mixture was extracted with EtOAc (100 mL×2). The combined organic layer was washed with brine (100 mL) and dried over Na₂SO₄ and concentrated under vacuum. The residue was purified using SGCC eluted with EtOAc/PE (0 to 80% EtOAc) to afford the title product (7.0 g) as yellow solid. LCMS (ESI): 349.25 [M+1]⁺.

### Step 2 methyl 2-bromo-4-(1-isopropyl-4-(trifluoromethyl)-1H-imidazol-2-yl)benzoate

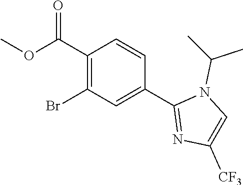 [SMILES: COC(=O)c1ccc(-c2nc(C(F)(F)F)cn2C(C)C)cc1Br]

A mixture of the product of Step 1 (5.0 g), 2-iodopropane (6.09 g) and Cs₂CO₃ (14.00 g) in DMF (30 mL) was stirred at 80° C. overnight. After being cooled to r.t., the reaction solution was poured into water (100 mL) and extracted with EtOAc (100 mL×2). The combined organic layer was washed with brine (100 mL), dried over Na₂SO₄ and concentrated under vacuum. The residue was purified using SGCC eluted with EtOAc/PE (0 to 80% EtOAc) to afford the title product (4.5 g) as a white solid. LCMS (ESI): 391.33 [M+1]⁺.

### Step 3 (2-bromo-4-(1-isopropyl-4-(trifluoromethyl)-1H-imidazol-2-yl)phenyl) methanol

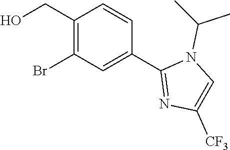 [SMILES: CC(C)n1cc(C(F)(F)F)nc1-c1ccc(CO)c(Br)c1]

To a solution of the product of Step 2 (2.5 g) in anhydrous DCM (30 mL) was added dropwise DIBAL-H (19 mL, 1M in hexane) at 0° C. over 10 min. The reaction solution was stirred at 0° C. for 30 min and then quenched by Na₂SO₄ decahydrate (5 g). The mixture was stirred at r.t. for 30 min and then filtered through a celite pad and washed with methanol/DCM (1/5, 100 mL). The filtrate was concentrated under vacuum and the residue was purified by SGCC eluted with EtOAc/PE (0 to 100% EtOAc) to afford the title product (2.0 g) as white solid. LCMS (ESI): 363.3 [M+1]⁺.

### Step 4 1-(2-bromo-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

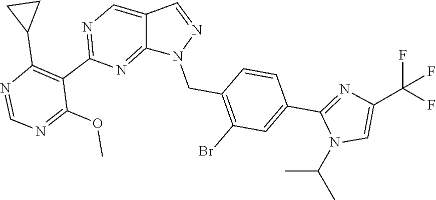 [SMILES: COc1ncnc(C2CC2)c1-c1ncc2cnn(Cc3ccc(-c4nc(C(F)(F)F)cn4C(C)C)cc3Br)c2n1]

To a mixture of the product of Step 9 in Example 3 (800 mg), the product of Step 3 (1.1 g) and Ph₃P (1.6 g) in THE (10 mL) was added dropwise DIAD (1.2 g) at r.t. The mixture was stirred at r.t. for 1 h. The reaction solution was concentrated and the residue was purified using SGCC with EtOAc/PE (0 to 80% EtOAc) to afford the title product (1.0 g) as white solid. LCMS (ESI): 613.4 [M+1]⁺.

### Step 5 ethyl (E)-3-(2-((6-(4-cyclopropyl-6-methoxypyrimidin-5-yl)-1H-pyrazolo [3,4-d]pyrimidin-1-yl)methyl)-5-(1-isopropyl-4-(trifluoromethyl)-1H-imidazol-2-yl)phenyl)acrylate

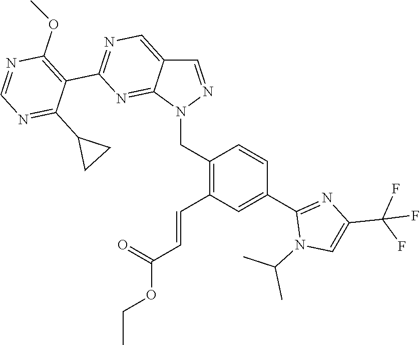 [SMILES: CCOC(=O)/C=C/c1cc(-c2nc(C(F)(F)F)cn2C(C)C)ccc1Cn1ncc2cnc(-c3c(OC)ncnc3C3CC3)nc21]

A mixture of the product of Step 4 (500 mg), ethyl (E)-3-(4,4,5,5-tetramethyl-1,3,2-dioxaborolan-2-yl)acrylate (553 mg), CsF (371 mg), Pd₂(dba)₃ (149 mg) and 2-(dicyclohexylphosphino)-2′,4′,6′-tri-i-propyl-1,1′-biphenyl (155 mg) in 1,4-dioxane/water (9.0 mL, 8/1) was degassed under vacuum and purged with Ar three times. The mixture was stirred at 90° C. overnight and then concentrated under reduced pressure. The residue was purified by SGCC eluted with EtOAc/PE (0 to 70% EtOAc) to afford the title product (350 mg) as white solid. LCMS (ESI): 633.6 [M+1]⁺.

### Step 6 ethyl 3-(2-((6-(4-cyclopropyl-6-methoxypyrimidin-5-yl)-1H-pyrazolo[3,4-d]pyrimidin-1-yl) methyl)-5-(1-isopropyl-4-(trifluoromethyl)-1H-imidazol-2-yl)phenyl)propanoate

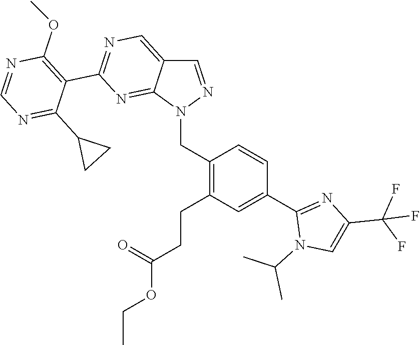 [SMILES: CCOC(=O)CCc1cc(-c2nc(C(F)(F)F)cn2C(C)C)ccc1Cn1ncc2cnc(-c3c(OC)ncnc3C3CC3)nc21]

To a mixture of the product of Step 5 (150 mg), water (43 mg) and palladium(II) acetate (16 mg) in DCM (3 mL) was added dropwise 4,4,5,5-tetramethyl-1,3,2-dioxaborolane (607 mg) at r.t. The mixture was stirred at r.t. for 1 h and then filtered through a celite pad and washed with methanol (20 mL). To the filtrate was added DIAD (300 mg) and the mixture was concentrated under reduced pressure. The residue was purified using SGCC eluted with EtOAc/PE (0 to 70% EtOAc) to afford the title product (150 mg) as white solid. LCMS (ESI): 635.7 [M+1]⁺.

### Step 7 3-(2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl) propan-1-ol

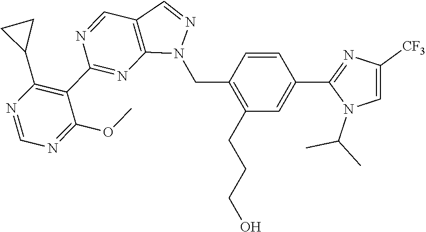 [SMILES: COc1ncnc(C2CC2)c1-c1ncc2cnn(Cc3ccc(-c4nc(C(F)(F)F)cn4C(C)C)cc3CCCO)c2n1]

To a solution of the product of Step 6 (150 mg) in anhydrous THE (2 mL) was added dropwise DIBAL-H (1 mL, 1M in hexane) at 0° C. The reaction solution was stirred at 0° C. for 30 min and then quenched with Na₂SO₄ decahydrate (1 g), followed by stirring at r.t. for 10 min. The resulting mixture was filtered through a celite pad and washed with methanol/DCM (1/5, 20 mL). The filtrate was treated with DIAD (300 mg) and the mixture was concentrated under reduced pressure. The residue was purified by SGCC eluted with EtOAc/PE (0 to 100% EtOAc) to afford the title product (100 mg) as yellow solid. LCMS (ESI): 593.7 [M+1]⁺.

### Step 8 1⁶-cyclopropyl-4⁴-(1-isopropyl-4-(trifluoromethyl)-1H-imidazol-2-yl)-2¹H-8-oxa-2(6,1)-pyrazolo[3,4-d]pyrimidina-1(5,4)-pyrimidina-4(1,2)-benzenacyclooctaphane

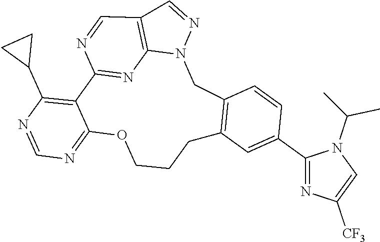 [SMILES: CC(C)n1cc(C(F)(F)F)nc1-c1ccc2c(c1)CCCOc1ncnc(C3CC3)c1-c1ncc3cnn(c3n1)C2]

Starting with the product of Step 7, the title product was obtained as white solid by following the same procedures as described in Step 13 and Step 14 of Example 3. LCMS (ESI): 561.60 [M+1]⁺. ¹H NMR (500 MHz, CDCl₃) δ 9.30 (s, 1H), 8.71 (s, 1H), 8.22 (s, 1H), 7.89 (d, 1H), 7.45 (s, 1H), 7.42 (s, 1H), 7.39 (d, 1H), 5.81 (brs, 2H), 4.58-4.45 (m, 3H), 3.76-3.39 (m, 2H), 2.61-2.54 (m, 1H), 2.17-2.01 (m, 2H), 1.45 (d, 6H), 1.37-1.33 (m, 2H), 1.11-1.03 (m, 2H).

### Example 10

### 1⁶-cyclopropyl-4⁴-(1-isopropyl-4-(trifluoromethyl)-1H-imidazol-2-yl)-2¹H-5-oxa-9-aza-2(6,1)-pyrazolo[3,4-d]pyrimidina-1(5,4)-pyrimidina-4(1,2)-benzenacyclononaphane

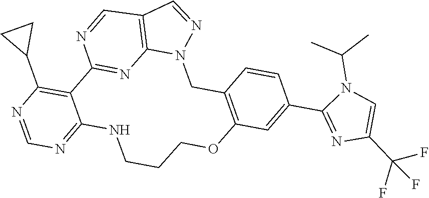

### Step 1 tert-butyl (3-(2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenoxy)propyl)carbamate

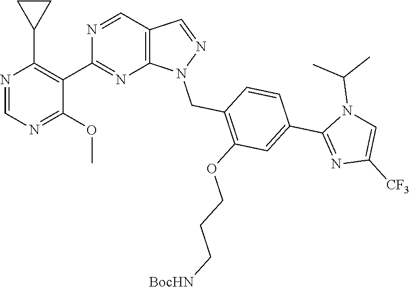 [SMILES: COc1ncnc(C2CC2)c1-c1ncc2cnn(Cc3ccc(-c4nc(C(F)(F)F)cn4C(C)C)cc3OCCCNC(=O)OC(C)(C)C)c2n1]

A mixture of Step 11 in Example 3 (0.2 g), tert-butyl (3-bromopropyl)carbamate (0.11 g), Cs₂CO₃ (0.23 g) in DMF (4 mL) was stirred at 60° C. for 0.5 h. The mixture was diluted with EtOAc (10 mL) and washed with brine (3×30 mL). The organic layer was concentrated and the residue was purified by SGCC (eluted with PE/EtOAc: from 100:0 to 1:1) to obtain the product as yellow solid (0.22 g). LCMS (ESI): 708.6 [M+1]⁺.

### Step 2 5-(1-(2-(3-aminopropoxy)-4-(1-isopropyl-4-(trifluoromethyl)-1H-imidazol-2-yl)benzyl)-1H-pyrazolo[3,4-d]pyrimidin-6-yl)-6-cyclopropylpyrimidin-4-ol

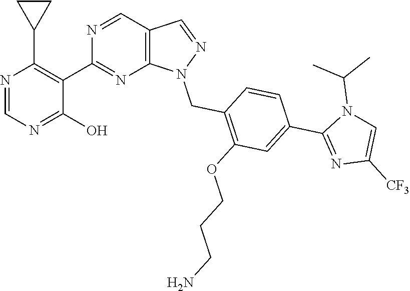 [SMILES: CC(C)n1cc(C(F)(F)F)nc1-c1ccc(Cn2ncc3cnc(-c4c(O)ncnc4C4CC4)nc32)c(OCCCN)c1]

A mixture of the product of Step 1 (0.22 g) in 6M HCl/THF (3/3 mL) was stirred at 80° C. for 1 h. The mixture was adjusted pH to 8 by addition of K₂CO₃ and extracted with EtOAc (10 mL). The organic layer was washed with brine (3×30 mL) and then concentrated to obtain the product as yellow solid (0.17 g). LCMS (ESI): 594.3 [M+1]⁺.

### Step 3 1⁶-cyclopropyl-4⁴-(1-isopropyl-4-(trifluoromethyl)-1H-imidazol-2-yl)-2¹H-5-oxa-9-aza-2(6,1)-pyrazolo[3,4-d]pyrimidina-1(5,4)-pyrimidina-4(1,2)-benzenacyclononaphane

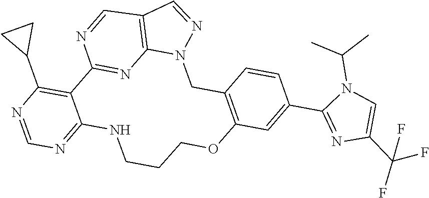

To a solution of the product of Step 2 (0.11 g), i-Pr₂NEt (0.14 g) in DMF (3 mL) was added benzotriazole-1-yl-oxytripyrrolidinophosphonium hexafluorophosphate (0.07 g). The reaction mixture was stirred at r.t. for 1 h and then diluted with EtOAc (3×30 mL) and washed with brine (10 mL). The organic layer was concentrated and purified by SGCC (eluted with PE/EtOAc: from 100:0 to 3:7) to obtain the product as white solid (5.3 mg). LCMS (ESI): 576.3 [M+1]⁺.

### Example 11

### 1⁶-cyclopropyl-4⁴-(1-isopropyl-4-(trifluoromethyl)-1H-imidazol-2-yl)-2¹H-5-oxa-8-aza-2(6,1)-pyrazolo[3,4-d]pyrimidina-1(5,4)-pyrimidina-4(1,2)-benzenacyclooctaphane

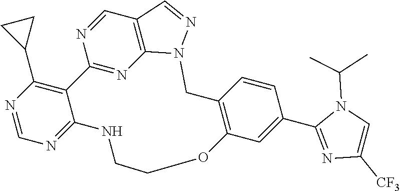 [SMILES: CC(C)n1cc(C(F)(F)F)nc1-c1ccc2c(c1)OCCNc1ncnc(C3CC3)c1-c1ncc3cnn(c3n1)C2]

Using tert-butyl (2-bromoethyl)carbamate as the starting material, the title compound was obtained by following the same procedures as described for Example 10. LCMS (ESI): 562.2 [M+1]⁺.

### Example 12

### 6′-cyclopropyl-4′-(1-isopropyl-4-(trifluoromethyl)-1H-imidazol-2-yl)spiro[cyclopropane-1,7′-5,9-dioxa-2(6,1)-pyrazolo[3,4-d]pyrimidina-1(5,4)-pyrimidina-4(1,2)-benzenacyclononaphane]

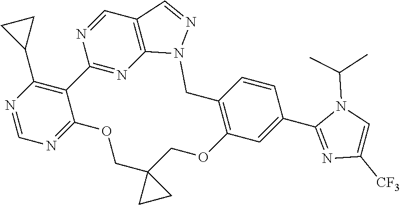 [SMILES: CC(C)n1cc(C(F)(F)F)nc1-c1ccc2c(c1)OCC1(CC1)COc1ncnc(C3CC3)c1-c1ncc3cnn(c3n1)C2]

Starting with the product of Step 11 in Example 3 and cyclopropane-1,1-diyldimethanol, the title product was obtained as white solid (13.1 mg) by following the same procedures as described for Example 7. LCMS (ESI): 603.6 [M+1]⁺.

### Example 13

### 1⁶-cyclopropyl-4⁴-(1-cyclopropyl-4-(trifluoromethyl)-1H-imidazol-2-yl)-2¹H-5,8-dioxa-2(6,1)-pyrazolo[3,4-d]pyrimidina-1(5,4)-pyrimidina-4(1,2)-benzenacyclooctaphane

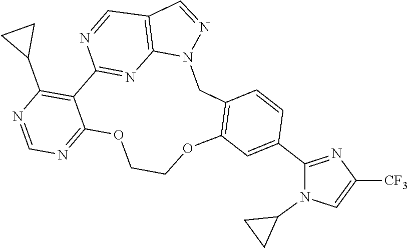

### Step 1 1-(2-(benzyloxy)-4-(1-cyclopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

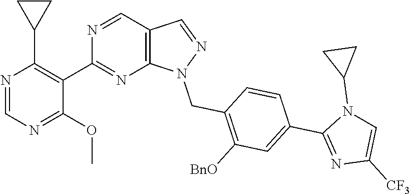 [SMILES: COc1ncnc(C2CC2)c1-c1ncc2cnn(Cc3ccc(-c4nc(C(F)(F)F)cn4C4CC4)cc3OCc3ccccc3)c2n1]

A mixture of the product of Step 9 in Example 3 (320 mg), 2-(3-(benzyloxy)-4-(chloromethyl)phenyl)-1-cyclopropyl-4-(trifluoromethyl)-1H-imidazole (585 mg) and Cs₂CO₃ (778 mg) in DMF (10 mL) was stirred at 60° C. for 2 h. The mixture was then poured into water (50 mL) and extracted with EtOAc (50 mL×2). The organic layer was dried over anhydrous Na₂SO₄ and concentrated under vacuum. The residue was purified by SGCC eluted with EtOAc/PE (0% to 100% EtOAc) to afford the title product as white solid (487 mg). LCMS (ESI): 639.6 [M+1]⁺.

### Step 2 5-(1-cyclopropyl-4-(trifluoromethyl)-1H-imidazol-2-yl)-2-((6-(4-cyclopropyl-6-methoxypyrimidin-5-yl)-1H-pyrazolo [3,4-d]pyrimidin-1-yl)methyl)phenol

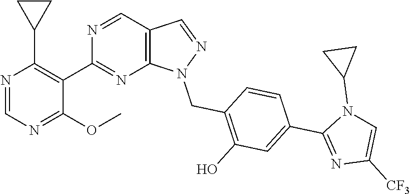 [SMILES: COc1ncnc(C2CC2)c1-c1ncc2cnn(Cc3ccc(-c4nc(C(F)(F)F)cn4C4CC4)cc3O)c2n1]

A solution of the product of Step 1 (400 mg) in TFA (10 mL) was stirred at 100° C. for 5 h. The reaction mixture was neutralized with aqueous NaHCO₃ and extracted with EtOAc (50 mL×2). The organic layer was washed with brine (10 mL), dried over anhydrous Na₂SO₄ and then concentrated under vacuum. The residue was purified using preparation TLC (EtOAc/PE=1/1) to afford the title compound as yellow solid (264 mg). LCMS (ESI): 549.6 [M+1]⁺.

### Step 3 1-(2-(2-((tert-butyldimethylsilyl)oxy)ethoxy)-4-(1-cyclopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

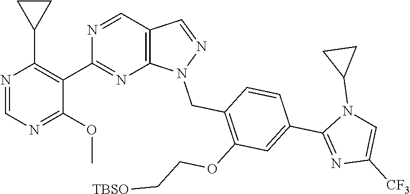 [SMILES: COc1ncnc(C2CC2)c1-c1ncc2cnn(Cc3ccc(-c4nc(C(F)(F)F)cn4C4CC4)cc3OCCO[Si](C)(C)C(C)(C)C)c2n1]

A mixture of the product of Step 2 (200 mg), (2-bromoethoxy)(tert-butyl)dimethylsilane (99 mg), KI (115 mg) and Cs₂CO₃ (226 mg) in DMF (30 mL) was stirred at 60° C. for 2 h. The mixture was poured into water (50 mL) and extracted with EtOAc (50 mL×2). The organic layer was dried over anhydrous Na₂SO₄ and concentrated under vacuum. The residue was purified by SGCC eluted with EtOAc/PE (0% to 100% EtOAc) to afford the title product as white solid (217 mg). LCMS (ESI): 737.2 [M+1]⁺.

### Step 4 1⁶-cyclopropyl-4⁴-(1-cyclopropyl-4-(trifluoromethyl)-1H-imidazol-2-yl)-2¹H-5,8-dioxa-2(6,1)-pyrazolo [3,4-d]pyrimidina-1(5,4)-pyrimidina-4(1,2)-benzenacyclooctaphane

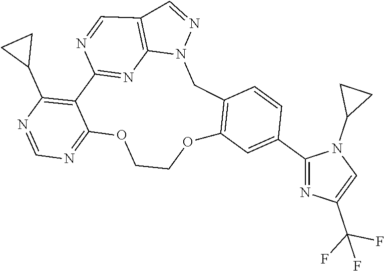 [SMILES: FC(F)(F)c1cn(C2CC2)c(-c2ccc3c(c2)OCCOc2ncnc(C4CC4)c2-c2ncc4cnn(c4n2)C3)n1]

Starting with the product of Step 3, the title product was obtained as a white solid by following the same procedures as described in Step 13 and Step 14 of Example 3. LCMS (ESI): 561.4 [M+1]⁺. ¹H NMR (500 MHz, CDCl₃) δ 9.28 (s, 1H), 8.74 (s, 1H), 8.17 (s, 1H), 7.82 (d, 1H), 7.51-7.48 (m, 1H), 7.37-7.31 (m, 1H), 6.02-5.64 (m, 2H), 5.84 (brs, 2H), 4.77-4.68 (m, 2H), 4.27 (s, 2H), 3.54-3.47 (m, 1H), 2.79-2.71 (m, 1H), 1.38-1.32 (m, 2H), 1.15-1.04 (m, 4H), 0.95-0.89 (m, 2H).

### Example 14

### 1⁶-cyclopropyl-4⁴-(1-isopropyl-4-(trifluoromethyl)-1H-imidazol-2-yl)-2¹H-8-oxa-5-aza-2(6,1)-pyrazolo[3,4-d]pyrimidina-1(5,4)-pyrimidina-4(1,2)-benzenacyclooctaphane

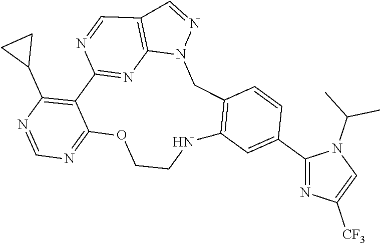 [SMILES: CC(C)n1cc(C(F)(F)F)nc1-c1ccc2c(c1)NCCOc1ncnc(C3CC3)c1-c1ncc3cnn(c3n1)C2]

Using 2-((tert-butyldimethylsilyl)oxy)acetaldehyde as the starting material in Step 7 of Example 8, the title compound was obtained following the same procedures as described for Example 8. LCMS (ESI): 562.4 [M+1]⁺.

### Example 15

### 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,2)-benzenacyclononaphan-6-one

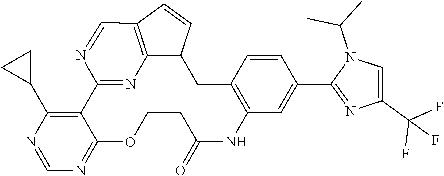 [SMILES: CC(C)n1cc(C(F)(F)F)nc1-c1ccc2c(c1)NC(=O)CCOc1ncnc(C3CC3)c1-c1ncc3c(n1)C(C=C3)C2]

### Step 1 5-(1-(2-amino-4-(1-isopropyl-4-(trifluoromethyl)-1H-imidazol-2-yl)benzyl)-1H-pyrazolo[3,4-d]pyrimidin-6-yl)-6-cyclopropylpyrimidin-4-ol

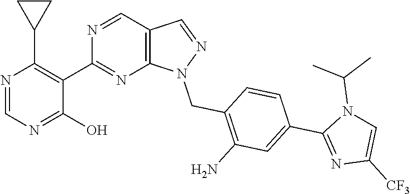 [SMILES: CC(C)n1cc(C(F)(F)F)nc1-c1ccc(Cn2ncc3cnc(-c4c(O)ncnc4C4CC4)nc32)c(N)c1]

A solution of the product of Step 6 in Example 8 (0.35 g) in 6 M HCl/THF (5/5 mL) was stirred at 80° C. for 1 h. The mixture was adjusted to pH=8 with K₂CO₃, extracted with EtOAc (3×30 mL). The combined organic layer was with brine (20 mL), concentrated to obtain the product as yellow solid (0.28 g). LCMS (ESI): 536.4 [M+1]⁺.

### Step 2 methyl 3-((5-(1-(2-amino-4-(1-isopropyl-4-(trifluoromethyl)-1H-imidazol-2-yl)benzyl)-1H-pyrazolo[3,4-d]pyrimidin-6-yl)-6-cyclopropylpyrimidin-4-yl)oxy)propanoate

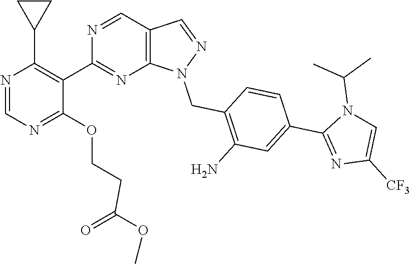 [SMILES: COC(=O)CCOc1ncnc(C2CC2)c1-c1ncc2cnn(Cc3ccc(-c4nc(C(F)(F)F)cn4C(C)C)cc3N)c2n1]

A solution of the product of Step 1 (0.26 g), methyl 3-bromopropanoate (0.16 g), Cs₂CO₃ (0.32 g) in DMF (4 mL) was stirred at 50° C. for 2 h. The mixture was extracted with EtOAc (10 mL). The organic layer was, concentrated and the residue was purified by SGCC (PE/EtOAc: from 100:0 to 1:9) to obtain the product as yellow oil (0.16 g). LCMS (ESI): 622.5 [M+1]⁺.

### Step 3 3-((5-(1-(2-amino-4-(1-isopropyl-4-(trifluoromethyl)-1H-imidazol-2-yl)benzyl)-1H-pyrazolo[3,4-d]pyrimidin-6-yl)-6-cyclopropylpyrimidin-4-yl)oxy)propanoic acid

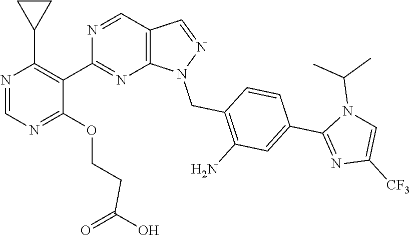 [SMILES: CC(C)n1cc(C(F)(F)F)nc1-c1ccc(Cn2ncc3cnc(-c4c(OCCC(=O)O)ncnc4C4CC4)nc32)c(N)c1]

A solution of the product of Step 2 (0.15 g), lithium hydroxide (0.2 g) THF/methanol/water (1.5/1/0.5 mL) was stirred at r.t. for 1 h. The mixture was adjusted pH to 4 with 6 M HCl solution and extracted with EtOAc (10 mL). The organic layer was washed with brine (3×30 mL) and concentrated to obtain the product as white solid (0.14 g). LCMS (ESI): 608.3 [M+1]⁺.

### Step 4 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,2)-benzenacyclononaphan-6-one

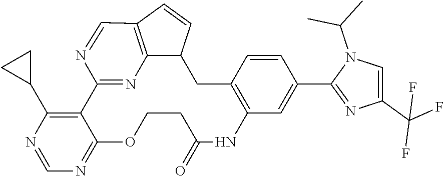 [SMILES: CC(C)n1cc(C(F)(F)F)nc1-c1ccc2c(c1)NC(=O)CCOc1ncnc(C3CC3)c1-c1ncc3c(n1)C(C=C3)C2]

To a solution of the product of Step 3 (0.12 g), 1-methylimidazole (0.10 g) in acetonitrile (5 mL), TCFH (0.11 g) was added and the reaction mixture was stirred at r.t. for 0.5 h. The reaction mixture was then diluted with EtOAc (10 mL) and washed with brine (3×30 mL). The organic combined layer was concentrated and the residue was purified by SGCC (DCM/methanol: from 100:0 to 10:1) to obtain the product as white solid (13.5 mg). LCMS (ESI): 590.3 [M+1]⁺.

### Example 16

### 1⁶-cyclopropyl-4⁴-(1-isopropyl-4-(trifluoromethyl)-1H-imidazol-2-yl)-2⁷,2⁸-dihydro-5,8-dioxa-2(2,8)-pyrido[2,3-d]pyrimidina-1(5,4)-pyrimidina-4(1,2)-benzenacyclooctaphan-2⁷-one

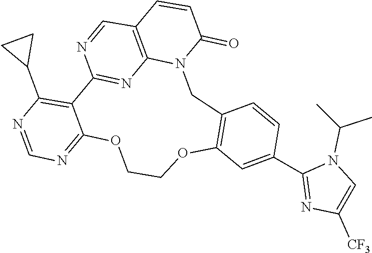 [SMILES: CC(C)n1cc(C(F)(F)F)nc1-c1ccc2c(c1)OCCOc1ncnc(C3CC3)c1-c1ncc3ccc(=O)n(c3n1)C2]

### Step 1 ethyl (E)-3-(4-amino-2-chloropyrimidin-5-yl)acrylate

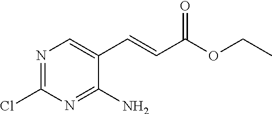

To a mixture of 4-amino-2-chloro-5-iodopyrimidine (500 mg) and ethyl acrylate (235 mg) in acetonitrile (30 mL) at r.t. were added Pd(OAc)₂ (44 mg) and triethylamine (396 mg). After being stirred at 90° C. for 18 h, the reaction mixture was poured into 100 mL ice-water and extracted with EtOAc (3×50 mL). The combined organic layer was washed with saturated brine (50 mL), dried over anhydrous Na₂SO₄ and concentrated. The residue was purified by SGCC (PE/EtOAc: from 100:0 to 10:1) to afford the title compound as yellow solid (350 mg). LCMS (ESI): 228.1 [M+1]⁺.

### Step 2 4-cyclopropyl-6-methoxy-5-(4,4,5,5-tetramethyl-1,3,2-dioxaborolan-2-yl)pyrimidine

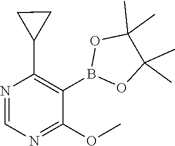 [SMILES: COc1ncnc(C2CC2)c1B1OC(C)(C)C(C)(C)O1]

A mixture of 5-bromo-4-cyclopropyl-6-methoxypyrimidine (10.0 g), bis(pinacolato)diboron (33.26 g), KI (12.85 g) and (Ph₃P)₂PdCl₂dichlorobis (1.53 g) in 1,4-dioxane (150 mL) was heated to reflux for 4 h under Ar. The reaction mixture was then concentrated and the residue was purified by SGCC (PE/EtOAc: from 100:0 to 1:20) to afford the title compound as white solid (15 g). LCMS (ESI): 277.5 [M+1]⁺.

### Step 3 ethyl (E)-3-(4-amino-4′-cyclopropyl-6′-methoxy-[2,5′-bipyrimidin]-5-yl)acrylate

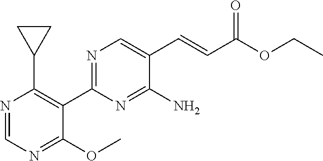 [SMILES: CCOC(=O)/C=C/c1cnc(-c2c(OC)ncnc2C2CC2)nc1N]

To a mixture of the product of Step 1 (200 mg) and the product of Step 2 (486 mg) in 1,2-dioxane/water (5/0.5 mL) at r.t. were added (dppf)PdCl₂—CH₂Cl₂ (71 mg) and KOAc (259 mg). After being stirred at 100° C. for 18 h, the reaction mixture was then poured into 100 mL ice-water and extracted with EtOAc (3×50 mL). The combined organic layer was washed with saturated brine (50 mL), dried over anhydrous Na₂SO₄ and concentrated. The residue was purified by SGCC (PE/EtOAc: from 100:0 to 10:1) to afford the title compound as yellow solid (41 mg). LCMS (ESI): 342.2 [M+1]⁺.

### Step 4 2-(4-cyclopropyl-6-methoxypyrimidin-5-yl)pyrido[2,3-d]pyrimidin-7(8H)-one

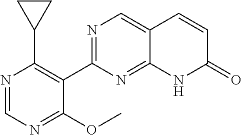 [SMILES: COc1ncnc(C2CC2)c1-c1ncc2ccc(=O)[nH]c2n1]

A mixture of the product of Step 3 (40 mg), sodium methoxide (127 mg) in methanol (5 mL) was stirred for 1 h at 50° C. The reaction mixture was then concentrated and the residue was purified by SGCC (PE/EtOAc=5:1) to afford the title compound as yellow solid (30 mg). LCMS (ESI): 298.4 [M+1]⁺.

### Step 5 2-(3-(benzyloxy)-4-(chloromethyl)phenyl)-1-isopropyl-4-(trifluoromethyl)-1H-imidazole

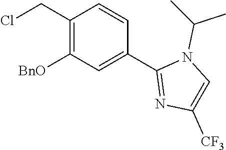 [SMILES: CC(C)n1cc(C(F)(F)F)nc1-c1ccc(CCl)c(OCc2ccccc2)c1]

The product of Step 6 in Example 3 (1.5 g) was dissolved in DCM (50 mL) and treated with thionyl chloride (15 mL) at 0° C. After stirring at r.t. for 2 h, the mixture was then concentrated in vacuo to give the product as yellow solid (1.8 g). LCMS (ESI): 409.2 [M+1]⁺.

### Step 6 8-(2-(benzyloxy)-4-(1-isopropyl-4-(trifluoromethyl)-1H-imidazol-2-yl)benzyl)-2-(4-cyclopropyl-6-methoxypyrimidin-5-yl)pyrido[2,3-d]pyrimidin-7(8H)-one

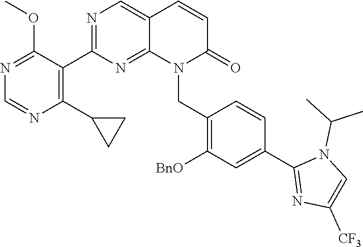 [SMILES: COc1ncnc(C2CC2)c1-c1ncc2ccc(=O)n(Cc3ccc(-c4nc(C(F)(F)F)cn4C(C)C)cc3OCc3ccccc3)c2n1]

A mixture of the product of Step 4 (100 mg), the product of Step 5 (133 mg) and Cs₂CO₃ (220 mg) in DMF (2 mL) was stirred at r.t. for 5 h. The mixture was poured into water (20 mL) and extracted with EtOAc (20 mL×3). The combined organic layer was dried over anhydrous Na₂SO₄ and concentrated under vacuum. The residue was purified by SGCC eluted with EtOAc/PE (0% to 100% EtOAc) to afford the title product as white solid (140 mg). LCMS (ESI): 668.2 [M+1]⁺.

### Step 7 2-(4-cyclopropyl-6-methoxypyrimidin-5-yl)-8-(2-hydroxy-4-(1-isopropyl-4-(trifluoromethyl)-1H-imidazol-2-yl)benzyl)pyrido[2,3-d]pyrimidin-7(8H)-one

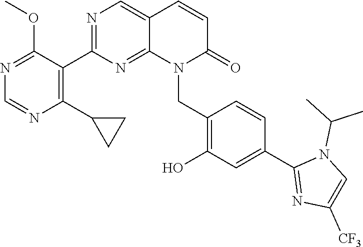 [SMILES: COc1ncnc(C2CC2)c1-c1ncc2ccc(=O)n(Cc3ccc(-c4nc(C(F)(F)F)cn4C(C)C)cc3O)c2n1]

A solution of the product of Step 6 (30 mg) in TFA (6 mL) was stirred at 100° C. for 5 h. The reaction mixture was neutralized with aqueous NaHCO₃ and extracted with EtOAc (2×20 mL). The combined organic layer was washed with brine (3×10 mL), dried over anhydrous Na₂SO₄ and then concentrated under vacuum. The residue was purified using preparation TLC (EtOAc/PE=1/1) to afford the title compound as yellow solid (19 mg). LCMS (ESI): 578.3 [M+1]⁺.

### Step 8 8-(2-(2-((tert-butyldimethylsilyl)oxy)ethoxy)-4-(1-isopropyl-4-(trifluoromethyl)-1H-imidazol-2-yl)benzyl)-2-(4-cyclopropyl-6-methoxypyrimidin-5-yl)pyrido[2,3-d]pyrimidin-7(8H)-one

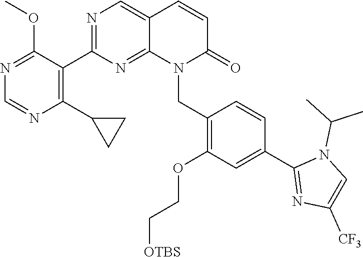 [SMILES: COc1ncnc(C2CC2)c1-c1ncc2ccc(=O)n(Cc3ccc(-c4nc(C(F)(F)F)cn4C(C)C)cc3OCCO[Si](C)(C)C(C)(C)C)c2n1]

A mixture of the product of Step 7 (200 mg), (2-bromoethoxy)(tert-butyl)dimethylsilane (99 mg), KI (115 mg) and Cs₂CO₃ (226 mg) in DMF (30 mL) was stirred at 60° C. for 2 h. The mixture was poured into water (20 mL) and extracted with EtOAc (20 mL×2). The organic combined layer was dried over anhydrous Na₂SO₄ and concentrated under vacuum. The residue was purified using SGCC eluted with EtOAc/PE (0% to 100% EtOAc) to afford the title product as white solid (217 mg). LCMS (ESI): 736.3 [M+1]⁺.

### Step 9 1⁶-cyclopropyl-4⁴-(1-isopropyl-4-(trifluoromethyl)-1H-imidazol-2-yl)-2⁷,2⁸-dihydro-5,8-dioxa-2(2,8)-pyrido[2,3-d]pyrimidina-1(5,4)-pyrimidina-4(1,2)-benzenacyclooctaphan-2⁷-one

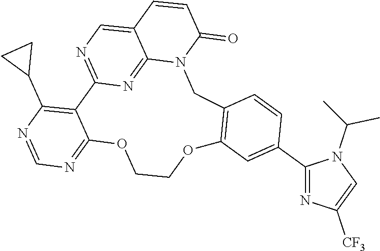 [SMILES: CC(C)n1cc(C(F)(F)F)nc1-c1ccc2c(c1)OCCOc1ncnc(C3CC3)c1-c1ncc3ccc(=O)n(c3n1)C2]

Starting with the product of Step 8, the title product was obtained as white solid by following the same procedures as described in Step 13 and Step 14 of Example 3. LCMS (ES+): 590.5 [M+1]⁺. ¹H NMR (500 MHz, CDCl₃) δ 8.98 (s, 1H), 8.71 (s, 1H), 8.09 (d, 1H), 7.72 (d, 1H), 7.43-7.40 (m, 1H), 7.14-7.11 (m, 1H), 7.04-6.99 (m, 1H), 6.85 (d, 1H), 6.4 (brs, 1H), 5.4 (brs, 1H), 4.80-4.72 (m, 2H), 4.64-4.56 (m, 1H), 4.48-4.35 (m, 2H), 2.43-2.34 (m, 1H), 1.46 (d, 6H), 1.38-1.30 (m, 2H), 1.09-0.97 (m, 2H).

### Example 17

### 1⁶-cyclopropyl-4⁴-(1-isopropyl-4-(trifluoromethyl)-1H-imidazol-2-yl)-2⁷,2⁸-dihydro-5,9-dioxa-2(2,8)-pyrido[2,3-d]pyrimidina-1(5,4)-pyrimidina-4(1,2)-benzenacyclononaphan-2⁷-one

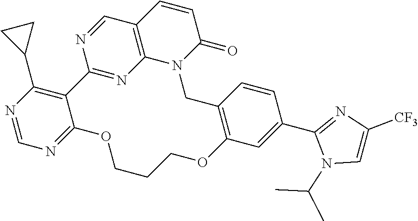 [SMILES: CC(C)n1cc(C(F)(F)F)nc1-c1ccc2c(c1)OCCCOc1ncnc(C3CC3)c1-c1ncc3ccc(=O)n(c3n1)C2]

Substituting (2-bromoethoxy)(tert-butyl)dimethylsilane with (2-bromoethoxy)(tert-butyl) dimethylsilane in the Step 8 of Example 16, the title compound was prepared by the same procedures as described for Example 16. LCMS (ESI): 604.6 [M+1]⁺. ¹H NMR (500 MHz, CDCl₃) δ 8.98 (s, 1H), 8.64 (s, 1H), 7.77-7.68 (m, 2H), 7.39 (s, 1H), 7.13-7.08 (m, 1H), 7.03-6.98 (m, 1H), 6.79 (d, 1H), 6.04-5.56 (m, 2H), 5.78 (brs, 2H), 4.67-4.51 (m, 3H), 4.18-4.03 (m, 2H), 2.31-2.17 (m, 3H), 1.43 (d, 6H), 1.33-1.27 (m, 2H), 1.06-0.95 (m, 2H).

### Example 18

### 1⁶-cyclopropyl-4⁴-(1-cyclopropyl-4-(trifluoromethyl)-1H-imidazol-2-yl)-2⁷,2⁸-dihydro-7-oxa-2(2,8)-pyrido[2,3-d]pyrimidina-1(5,4)-pyrimidina-4(1,2)-benzenacycloheptaphan-2⁷-one

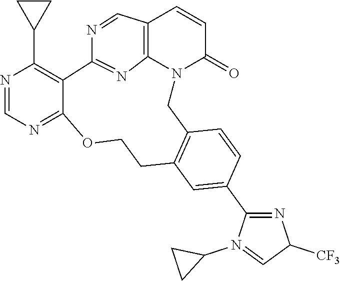

### Step 1 2-(isochroman-6-yl)-1-isopropyl-4-(trifluoromethyl)-1H-imidazole

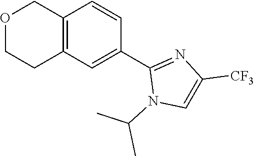 [SMILES: CC(C)n1cc(C(F)(F)F)nc1-c1ccc2c(c1)CCOC2]

To a mixture of the product of 2-(isochroman-6-yl)-4,4,5,5-tetramethyl-1,3,2-dioxaborolane (500 mg) and the product of Step 2 in Example 8 (593 mg) in 1,2-dioxane/water (5/0.5 mL) at r.t. were added XPhos Pd G1 (151 mg) and Na₃PO₄ (1.2 g). After being stirred at 100° C. for 8 h, the reaction mixture was poured into 100 mL ice-water and extracted EtOAc (3×50 mL). The combined organic layer was washed with saturated brine (50 mL), dried over anhydrous Na₂SO₄ and concentrated. The residue was purified by SGCC (PE/EtOAc: from 100:0 to 10:1) to afford the title compound as yellow solid (41 mg). LCMS (ESI): 311.2 [M+1]⁺.

### Step 2 2-(bromomethyl)-5-(1-isopropyl-4-(trifluoromethyl)-1H-imidazol-2-yl)phenEtOAc

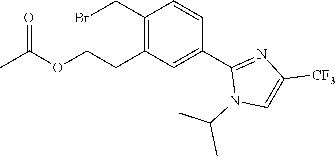 [SMILES: CC(=O)OCCc1cc(-c2nc(C(F)(F)F)cn2C(C)C)ccc1CBr]

A mixture of the product of Step 1 (500 mg) in 33% of hydrobromic acid in acetic acid (10 mL) was stirred at 100° C. for 3 h. The reaction mixture was neutralized with aqueous NaHCO₃ and extracted with EtOAc (50×3 mL). The combined organic layer was washed with brine (50 mL), dried over anhydrous Na₂SO₄ and concentrated to afford the title compound as yellow solid (435 mg). LCMS (ESI): 433.1 [M+1]⁺.

### Step 3 2-((2-(4-cyclopropyl-6-methoxypyrimidin-5-yl)-7-oxopyrido[2,3-d]pyrimidin-8(7H)-yl) methyl)-5-(1-isopropyl-4-(trifluoromethyl)-1H-imidazol-2-yl)phenethyl acetate

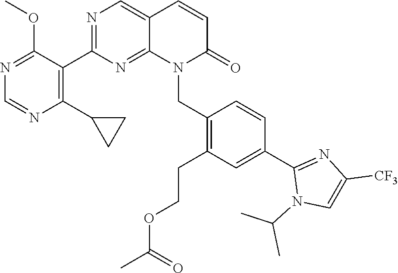 [SMILES: COc1ncnc(C2CC2)c1-c1ncc2ccc(=O)n(Cc3ccc(-c4nc(C(F)(F)F)cn4C(C)C)cc3CCOC(C)=O)c2n1]

A mixture of the products of Step 2 (150 mg), Step 4 in Example 16 (100 mg) and Cs₂CO₃ (133 mg) in DMF (20 mL) was stirred at r.t. for 5 h. The mixture was poured into water (20 mL) and extracted with EtOAc (20 mL×2). The combined organic layer was dried over anhydrous Na₂SO₄ and concentrated under vacuum. The residue was purified by silica gel chromatography column eluted with EtOAc/PE (0% to 100% EtOAc) to afford the title product as white solid (140 mg). LCMS (ESI): 648.3 [M+1]⁺.

### Step 4 1⁶-cyclopropyl-4⁴-(1-isopropyl-4-(trifluoromethyl)-1H-imidazol-2-yl)-2⁷,2⁸-dihydro-7-oxa-2 (2,8)-pyrido[2,3-d]pyrimidina-1(5,4)-pyrimidina-4(1,2)-benzenacycloheptaphan-27-one

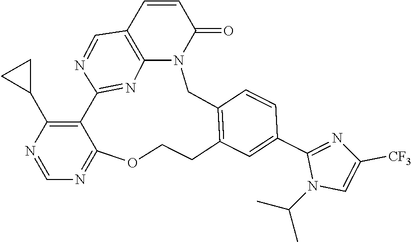 [SMILES: CC(C)n1cc(C(F)(F)F)nc1-c1ccc2c(c1)CCOc1ncnc(C3CC3)c1-c1ncc3ccc(=O)n(c3n1)C2]

Starting with the product of Step 3, the title product was obtained as a white solid by following the same procedures as described in Step 13 and Step 14 of Example 3. LCMS (ES+): 572.5[M+1]⁺.

### Example 19

### 1⁶-cyclopropyl-4⁴-(1-isopropyl-4-(trifluoromethyl)-1H-imidazol-2-yl)-2⁷,2⁸-dihydro-5-oxa-9-aza-2(2,8)-pyrido[2,3-d]pyrimidina-1(5,4)-pyrimidina-4(1,2)-benzenacyclononaphan-2⁷-one

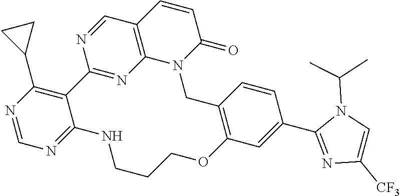 [SMILES: CC(C)n1cc(C(F)(F)F)nc1-c1ccc2c(c1)OCCCNc1ncnc(C3CC3)c1-c1ncc3ccc(=O)n(c3n1)C2]

### Step 1 8-(2-(benzyloxy)-4-(1-isopropyl-4-(trifluoromethyl)-1H-imidazol-2-yl)benzyl)-2-(4-cyclopropyl-6-hydroxypyrimidin-5-yl)pyrido[2,3-d]pyrimidin-7(8H)-one

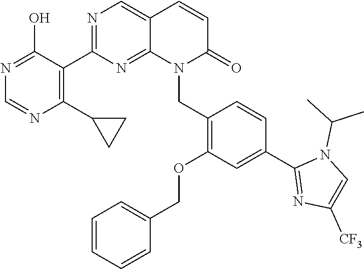 [SMILES: CC(C)n1cc(C(F)(F)F)nc1-c1ccc(Cn2c(=O)ccc3cnc(-c4c(O)ncnc4C4CC4)nc32)c(OCc2ccccc2)c1]

A mixture of the product of Step 6 in Example 16 (50 mg) in hydrochloric acid (6 mL, 3 M) and methanol (1 mL) was stirred at 100° C. for 3 h. The reaction mixture was neutralized with aqueous NaHCO₃ and extracted with EtOAc (50×3 mL). The organic layer was washed with brine (50 mL), dried over anhydrous Na₂SO₄ and then concentrated to afford the title compound as yellow solid (38 mg). LCMS (ESI): 654.3 [M+1]⁺.

### Step 2 8-(2-(benzyloxy)-4-(1-isopropyl-4-(trifluoromethyl)-1H-imidazol-2-yl)benzyl)-2-(4-chloro-6-cyclopropylpyrimidin-5-yl)pyrido[2,3-d]pyrimidin-7(8H)-one

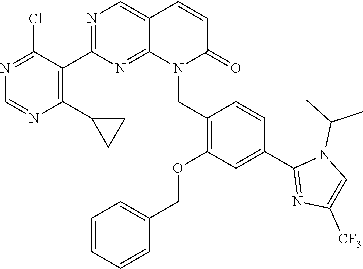 [SMILES: CC(C)n1cc(C(F)(F)F)nc1-c1ccc(Cn2c(=O)ccc3cnc(-c4c(Cl)ncnc4C4CC4)nc32)c(OCc2ccccc2)c1]

A mixture of the product of Step 1 (20 mg), phosphorus oxychloride (0.03 mL) and DMF (0.1 mL) in acetonitrile (10 mL) was stirred at 100° C. for 3 h. The reaction mixture was concentrated to afford the title compound as yellow solid (19 mg). LCMS (ESI): 672.2 [M+1]⁺.

### Step 3 8-(2-(benzyloxy)-4-(1-isopropyl-4-(trifluoromethyl)-1H-imidazol-2-yl)benzyl)-2-(4-((3-((tert-butyldimethylsilyl)oxy)propyl)amino)-6-cyclopropylpyrimidin-5-yl)pyrido[2,3-d]pyrimidin-7(8H)-one

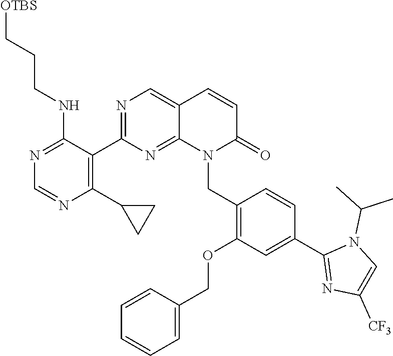 [SMILES: CC(C)n1cc(C(F)(F)F)nc1-c1ccc(Cn2c(=O)ccc3cnc(-c4c(NCCCO[Si](C)(C)C(C)(C)C)ncnc4C4CC4)nc32)c(OCc2ccccc2)c1]

A mixture of the product of Step 3 (150 mg), 3-((tert-butyldimethylsilyl)oxy)propan-1-amine (63 mg) and i-Pr₂NEt (75 mg) in THF (10 mL) was stirred at r.t. for 1 h. The mixture was poured into water (20 mL) and extracted with EtOAc (20 mL×2). The organic layer was dried over anhydrous Na₂SO₄ and concentrated under vacuum. The residue was purified using SGCC eluted with EtOAc/PE (0% to 100% EtOAc) to afford the title product as white solid (134 mg). LCMS (ESI): 825.4 [M+1]⁺.

### Step 4 2-(4-cyclopropyl-6-((3-hydroxypropyl)amino)pyrimidin-5-yl)-8-(2-hydroxy-4-(1-isopropyl-4-(trifluoromethyl)-1H-imidazol-2-yl)benzyl)pyrido[2,3-d]pyrimidin-7(8H)-one

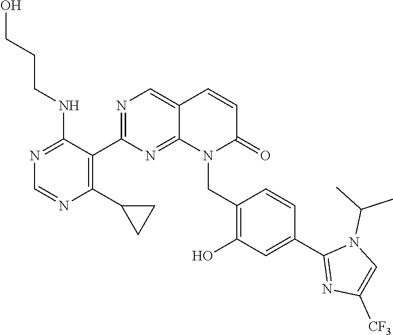 [SMILES: CC(C)n1cc(C(F)(F)F)nc1-c1ccc(Cn2c(=O)ccc3cnc(-c4c(NCCCO)ncnc4C4CC4)nc32)c(O)c1]

A mixture of the product of Step 3 (120 mg) in TFA (6 mL) was stirred at 100° C. for 12 h. The reaction mixture was neutralized with aqueous NaHCO₃, and then extracted with EtOAc (50×3 mL). The organic layer was washed with brine (50 mL), dried over anhydrous Na₂SO₄ and then concentrated to afford the title compound as yellow solid (120 mg). LCMS (ESI): 621.3 [M+1]⁺.

### Step 5 1⁶-cyclopropyl-4⁴-(1-isopropyl-4-(trifluoromethyl)-1H-imidazol-2-yl)-2⁷,2⁸-dihydro-5-oxa-9-aza-2(2,8)-pyrido[2,3-d]pyrimidina-1(5,4)-pyrimidina-4(1,2)-benzenacyclononaphan-2⁷-one

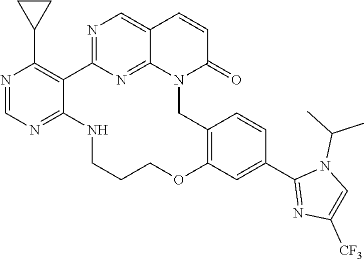 [SMILES: CC(C)n1cc(C(F)(F)F)nc1-c1ccc2c(c1)OCCCNc1ncnc(C3CC3)c1-c1ncc3ccc(=O)n(c3n1)C2]

To a mixture of Step 4 (50 mg) and Ph₃P (111 mg) in THE (10 mL) at 0° C. was added DIAD (98 mg). The reaction mixture was then stirred at 0° C. for 30 min. The reaction mixture was poured into 100 mL ice-water, extracted 3×50 mL EtOAc. The combined organic layer was washed with 50 mL saturated brine, dried over anhydrous Na₂SO₄ and concentrated. The residue was purified by SGCC (PE/EtOAc: from 100:0 to 2:1) to afford the title compound as white solid (19 mg). LCMS (ES+): 603.4 [M+1]⁺.

### Example 20

### 1⁶-cyclopropyl-4⁴-(1-isopropyl-4-(trifluoromethyl)-1H-imidazol-2-yl)-2⁷,2⁸-dihydro-9-oxa-5-aza-2(2,8)-pyrido[2,3-d]pyrimidina-1(5,4)-pyrimidina-4(1,2)-benzenacyclononaphan-2⁷-one

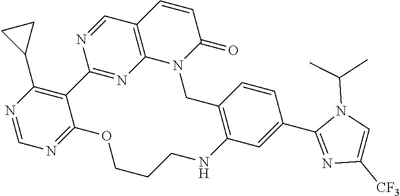 [SMILES: CC(C)n1cc(C(F)(F)F)nc1-c1ccc2c(c1)NCCCOc1ncnc(C3CC3)c1-c1ncc3ccc(=O)n(c3n1)C2]

### Step 1 2-(4-cyclopropyl-6-methoxypyrimidin-5-yl)-8-(4-(1-isopropyl-4-(trifluoromethyl)-1H-imidazol-2-yl)-2-nitrobenzyl)pyrido[2,3-d]pyrimidin-7(8H)-one

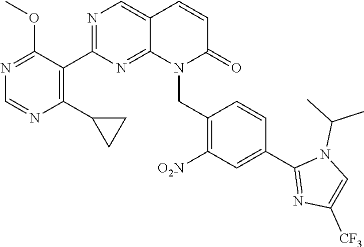 [SMILES: COc1ncnc(C2CC2)c1-c1ncc2ccc(=O)n(Cc3ccc(-c4nc(C(F)(F)F)cn4C(C)C)cc3[N+](=O)[O-])c2n1]

A mixture of the product of the product of Step 4 in Example 8 (795 mg), the product of Step 4 in Example 16 (750 mg) and Cs₂CO₃ (1.7 g) in DMF (30 mL) was stirred at r.t. for 5 h. The mixture was poured into water (20 mL) and extracted with EtOAc (20 mL×2). The organic layer was dried over anhydrous Na₂SO₄ and concentrated under vacuum. The residue was purified by SGCC eluted with EtOAc/PE (0% to 100% EtOAc) to afford the title product as white solid (800 mg). LCMS (ESI): 607.3 [M+1]⁺.

### Step 2 8-(2-amino-4-(1-isopropyl-4-(trifluoromethyl)-1H-imidazol-2-yl)benzyl)-2-(4-cyclopropyl-6-methoxypyrimidin-5-yl)pyrido[2,3-d]pyrimidin-7(8H)-one

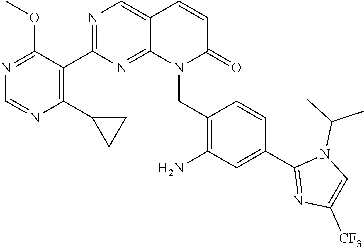 [SMILES: COc1ncnc(C2CC2)c1-c1ncc2ccc(=O)n(Cc3ccc(-c4nc(C(F)(F)F)cn4C(C)C)cc3N)c2n1]

A mixture of the product of Step 1 (100 mg), Zn powder (54 mg) and saturated ammonium chloride (0.2 mL) in methanol (30 ml) was stirred at 80° C. for 2 h. The mixture was filtered through celite pad and the celite pad was washed with methanol (20.0 mL×5). The filtrate was concentrated and the residue was purified by SGCC eluted with EtOAc/PE (0% to 100% EtOAc) to afford the title product as white solid (60 mg). LCMS (ESI): 577.2 [M+1]⁺.

### Step 3 8-(2-((3-((tert-butyldimethylsilyl)oxy)propyl)amino)-4-(1-isopropyl-4-(trifluoromethyl)-1H-imidazol-2-yl)benzyl)-2-(4-cyclopropyl-6-methoxypyrimidin-5-yl)pyrido[2,3-d]pyrimidin-7(8H)-one

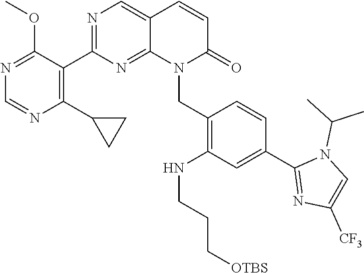 [SMILES: COc1ncnc(C2CC2)c1-c1ncc2ccc(=O)n(Cc3ccc(-c4nc(C(F)(F)F)cn4C(C)C)cc3NCCCO[Si](C)(C)C(C)(C)C)c2n1]

A solution of the product of step 2 (120 mg), 3-((tert-butyldimethylsilyl)oxy)propanal (47 mg), NaBH(OAc)₃ (221 mg) in DCM (30 mL) was stirred at r.t. for 1 h. The mixture was then concentrated and the residue was purified by SGCC (PE/EtOAc: from 100:0 to 3:7) to afford the title compound as yellow oil (116 mg). LCMS (ESI): 749.4 [M+1]⁺.

### Step 4 1⁶-cyclopropyl-4⁴-(1-isopropyl-4-(trifluoromethyl)-1H-imidazol-2-yl)-2⁷,2⁸-dihydro-9-oxa-5-aza-2(2,8)-pyrido[2,3-d]pyrimidina-1(5,4)-pyrimidina-4(1,2)-benzenacyclononaphan-2⁷-one

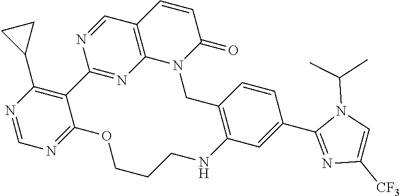 [SMILES: CC(C)n1cc(C(F)(F)F)nc1-c1ccc2c(c1)NCCCOc1ncnc(C3CC3)c1-c1ncc3ccc(=O)n(c3n1)C2]

Starting with the product of Step 3, the title product was obtained as a white solid by following the same procedures as described in Step 13 and Step 14 of Example 3. LCMS (ES+): 603.5 [M+1]⁺.

### Example 21

### (Z)-1⁶-cyclopropyl-4⁴-(1-cyclopropyl-4-(trifluoromethyl)-1H-imidazol-2-yl)-5,8-dioxa-2(2,7)-pyrrolo[2,1-f][1,2,4]triazina-1(5,4)-pyrimidina-4(1,2)-benzenacyclooctaphane

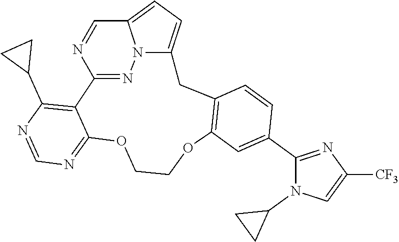 [SMILES: FC(F)(F)c1cn(C2CC2)c(-c2ccc3c(c2)OCCOc2ncnc(C4CC4)c2-c2ncc4ccc(n4n2)C3)n1]

### Step 1 methyl 4-(1-cyclopropyl-4-(trifluoromethyl)-1H-imidazol-2-yl)-2-methoxybenzoate

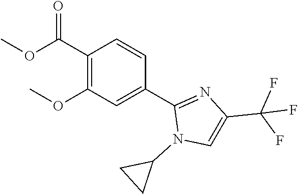 [SMILES: COC(=O)c1ccc(-c2nc(C(F)(F)F)cn2C2CC2)cc1OC]

A mixture of the product of Step 2 of Example 3 (5.0 g), cyclopropylboronic acid (2.7 g), Cu(OAc)₂ (2.8 g), 2,2′-bipyridine (2.4 g) and Na₂CO₃ (3.30 g) in 1,2-dichloroethane (100 mL) was stirred at 80° C. for 24 h under air. The reaction solution was poured into saturated ammonium chloride (100 mL) and extracted with EtOAc (100 mL×2). The combined organic layer was washed with brine (100 mL) and dried over Na₂SO₄ and concentrated under vacuum. The residue was purified by SGCC eluted with EtOAc/PE (0 to 60% EtOAc) to afford product the title product (4.7 g) as white solid. LCMS (ESI): 341.4 [M+1]⁺.

### Step 2 (2-(benzyloxy)-4-(1-cyclopropyl-4-(trifluoromethyl)-1H-imidazol-2-yl) phenyl)methanol

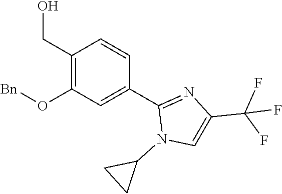 [SMILES: OCc1ccc(-c2nc(C(F)(F)F)cn2C2CC2)cc1OCc1ccccc1]

Starting with the product of Step 1 (4.7 g), the title product was obtained as a white solid (2 g) by following the same procedures as described in Step 4, Step 5 and Step 6 of Example 3. LCMS (ESI): 389.5 [M+1]⁺.

### Step 3 2-(3-(benzyloxy)-4-(chloromethyl)phenyl)-1-cyclopropyl-4-(trifluoromethyl)-1H-imidazole

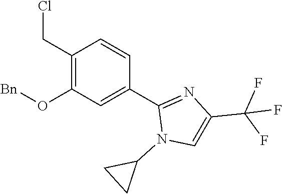 [SMILES: FC(F)(F)c1cn(C2CC2)c(-c2ccc(CCl)c(OCc3ccccc3)c2)n1]

A solution of the product of Step 2 (2.0 g) in DCM/thionyl chloride (20 mL, 5/1) was stirred at r.t. for 30 min. The reaction solution was concentrated under vacuum and the residue was dissolved in EtOAc (60 mL). The solution was washed with saturated NaHCO₃ (60 mL), brine (60 mL), dried over anhydrous Na₂SO₄ and concentrated to afford the title product (1.6 g) as white solid. LCMS (ESI): 407.6 [M+1]⁺.

### Step 4 2-chloropyrrolo[2,1-f][1,2,4]triazine

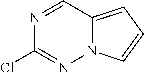

A mixture of 2,4-dichloropyrrolo[2,1-f][1,2,4]triazine (1.0 g) and sodium borohydride (305 mg) in methanol (30 mL) was stirred at r.t. for 18 h. The reaction mixture was then poured into 100 mL EtOAc and washed with 3×100 mL ammonium chloride (aq.). The organic layer was washed with 50 mL saturated brine, dried over anhydrous Na₂SO₄ and concentrated. The residue was dissolved in DCM (30 mL) and treated with 2,3-dichloro-5,6-dicyano-1,4-benzoquinone (1.8 g). After stirring at r.t. for 18 h, the reaction mixture was poured into 100 mL ice-water, treated with potassium bicarbonate (aq.) and extracted with EtOAc (3×50 mL). The combined organic layer was washed with saturated brine (50 mL), dried over anhydrous Na₂SO₄ and concentrated. The residue was purified by SGCC (PE/EtOAc: from 100:0 to 10:1) to afford the title compound as yellow solid (540 mg). LCMS (ESI): 154.2 [M+1]⁺.

### Step 5 2-(4-cyclopropyl-6-methoxypyrimidin-5-yl)pyrrolo[2,1-f][1,2,4]triazine

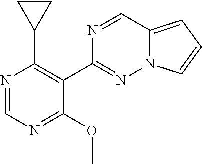 [SMILES: COc1ncnc(C2CC2)c1-c1ncc2cccn2n1]

To a mixture of the product of Step 1 (540 mg) and the product of Step 2 in Example 16 (1.02 g) in 1,2-dioxane/water (20/2 mL) were added Pd₂(dba)₃ (323 mg), 2-(dicyclohexylphosphino)-2,4,6-triisopropylbiphenyl (337 mg) and CsF (2.6 g) at r.t. After stirring at 100° C. for 18 h, the reaction mixture was poured into 100 mL of ice-water, and extracted 3×50 mL EtOAc. The combined organic layer was washed with 50 mL saturated brine, dried over anhydrous Na₂SO₄ and concentrated. The residue was purified by SGCC (PE/EtOAc: from 100:0 to 10:1) to afford the title compound as yellow solid (690 mg). LCMS (ESI): 268.4 [M+1]⁺.

### Step 6 7-bromo-2-(4-cyclopropyl-6-methoxypyrimidin-5-yl)pyrrolo[2,1-f][1,2,4]triazine

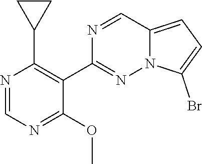 [SMILES: COc1ncnc(C2CC2)c1-c1ncc2ccc(Br)n2n1]

A mixture of the product of Step 2 (650 mg) and N-bromosuccinimide (477 mg) in acetonitrile (10 mL) was stirred at r.t. for 18 h. The reaction mixture was then treated with 10 mL water and extracted with EtOAc (50×3 mL). The combined organic layer was washed with 100 mL saturated brine, dried over anhydrous Na₂SO₄ and concentrated. The residue was purified by SGCC (PE/EtOAc: from 100:0 to 5:1) to afford the title compound as yellow solid (350 mg). LCMS (ESI): 346.3 [M+1]⁺.

### Step 7 7-(2-(benzyloxy)-4-(1-cyclopropyl-4-(trifluoromethyl)-1H-imidazol-2-yl)benzyl)-2-(4-cyclopropyl-6-methoxypyrimidin-5-yl)pyrrolo[2,1-f][1,2,4]triazine

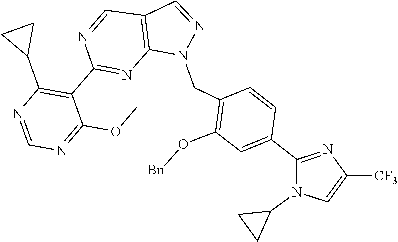

A solution of the product of Step 3 (940 mg) and sodium 3-methoxy-3-oxopropane-1-sulfinate (443 mg) in DMSO (10.0 mL) was stirred at 45° C. for 1 h under Ar and then cooled to r.t. To the reaction mixture were added the product of Step 6 (500 mg), K₂CO₃ (399 mg), di-tert-butyl(methyl)phosphonium tetrafluoroborate (143 mg) and Pd(OAc)₂ (65 mg). After stirring at 130° C. for 3 h, the reaction mixture was cooled to r.t. and diluted with EtOAc (80 mL). The solution was washed with brine (50 mL×2), dried over anhydrous Na₂SO₄ and concentrated under vacuum. The residue was purified by SGCC eluted with EtOAc/PE (0 to 60% EtOAc) to afford the title product (660 mg) as yellow solid. LCMS (ESI): 638.6 [M+1]⁺.

### Step 8 5-(1-cyclopropyl-4-(trifluoromethyl)-1H-imidazol-2-yl)-2-((2-(4-cyclopropyl-6-methoxypyrimidin-5-yl)pyrrolo[2,1-f][1,2,4]triazin-7-yl)methyl)phenol

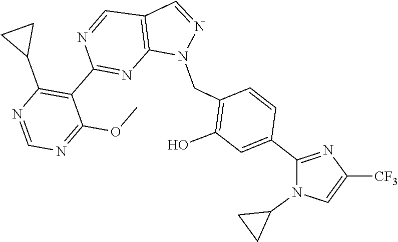

Starting with the product of Step 7 (300 mg), the title product was obtained as a white solid (200 mg) by following the same procedures as described in Step 11 of Example 3. LCMS (ESI): 548.6 [M+1]⁺.

### Step 9 7-(2-(2-((tert-butyldimethylsilyl)oxy)ethoxy)-4-(1-cyclopropyl-4-(trifluoromethyl)-1H-imidazol-2-yl)benzyl)-2-(4-cyclopropyl-6-methoxypyrimidin-5-yl)pyrrolo[2,1-f][1,2,4]triazine

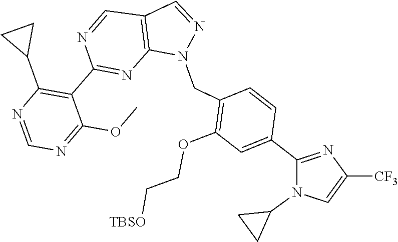

A mixture of the product of Step 8 (180 mg), (2-bromoethoxy)(tert-butyl)dimethylsilane (157 mg), potassium iodide (82 mg) and Cs₂CO₃ (321 mg) in DMF (6 mL) was stirred at 80° C. for 4 h. The reaction solution was poured into water (50 mL) and extracted with EtOAc (50 mL×2). The combined organic layer was washed with brine (50 mL) and dried over Na₂SO₄ and concentrated under vacuum. The residue was purified using SGCC eluted with EtOAc/PE (0 to 50% EtOAc) to afford product the title product (200 mg) as white solid. LCMS (ESI): 706.8 [M+1]⁺.

### Step 10 (Z)-1⁶-cyclopropyl-4⁴-(1-cyclopropyl-4-(trifluoromethyl)-1H-imidazol-2-yl)-5,8-dioxa-2(2,7)-pyrrolo[2,1-f][1,2,4]triazina-1(5,4)-pyrimidina-4(1,2)-benzenacyclooctaphane

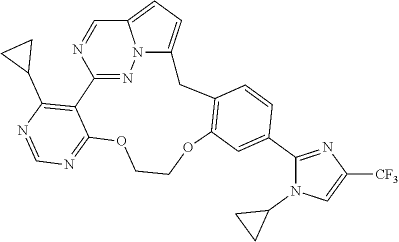 [SMILES: FC(F)(F)c1cn(C2CC2)c(-c2ccc3c(c2)OCCOc2ncnc(C4CC4)c2-c2ncc4ccc(n4n2)C3)n1]

Starting with the product of Step 9 (200 mg), the title product was obtained as a white solid (20 mg) by following the same procedures as described in Step 13 and Step 14 of Example 3. LCMS (ESI): 560.56 [M+1]⁺. ¹H NMR (500 MHz, CDCl₃) δ 8.98 (s, 1H), 8.71 (s, 1H), 7.57 (d, 1H), 7.44 (d, 1H), 7.33 (s, 1H), 7.29 (s, 1H), 6.96 (d, 1H), 6.88 (d, 1H), 4.75-4.68 (m, 2H), 4.60 (s, 2H), 4.33-4.25 (m, 2H), 3.53-3.45 (m, 1H), 2.63-2.56 (m, 1H), 1.35-1.32 (m, 2H), 1.11-1.06 (m, 4H), 0.93-0.91 (m, 2H).

### Example 22

### 1⁶-cyclopropyl-4⁴-(1-isopropyl-4-(trifluoromethyl)-1H-imidazol-2-yl)-2¹-methyl-2¹H-5,8-dioxa-2(5,3)-pyrazolo[4,3-d]pyrimidina-1(5,4)-pyrimidina-4(1,2)-benzenacyclooctaphane

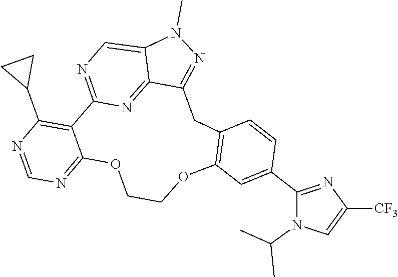

### Step 1 2-chloro-4-methylpyrimidin-5-amine

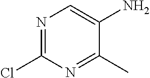

A mixture of 2,4-dichloro-6-methylpyrimidin-5-amine (10.0 g), Et₃N (2.56 g) and palladium/C (356 mg, 10%) in ethanol (100 mL) was stirred at r.t. under H₂ atmosphere overnight. The reaction solution was filtered and concentrated under vacuum. The residue was dissolved in EtOAc (150 mL) and then filtered. The filtrate was concentrated under vacuum to afford the title compound as brown solid (8.0 g, crude). LCMS (ESI): 144.5 [M+1]⁺.

### Step 2 N-(2-chloro-4-methylpyrimidin-5-yl)acetamide

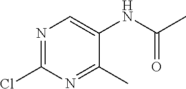

To a solution of the product of Step 1 (7.5 g) in DCM (20.0 mL) was added acetic anhydride (10.5 g) at room temperature. The solution was stirred at r.t. for 2 h. The reaction solution was then concentrated under vacuum and the residue was purified using SGCC eluted with EtOAc/PE (3/2) to afford the title compound as yellow solid (7.8 g). LCMS (ESI): 186.4 [M+1]⁺.

### Step 3 N-(4′-cyclopropyl-6′-methoxy-4-methyl-[2,5′-bipyrimidin]-5-yl)acetamide

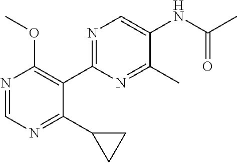 [SMILES: COc1ncnc(C2CC2)c1-c1ncc(NC(C)=O)c(C)n1]

A mixture of the product of Step 2 (2.0 g), 4-cyclopropyl-6-methoxy-5-(4,4,5,5-tetramethyl-1,3,2-dioxaborolan-2-yl)pyrimidine (7.94 g), K₂CO₃ (4.47 g) and Pd(dppf)Cl₂ DCM complex (352 mg) in dioxane/water (30 mL, 10/1) was stirred at 90° C. under Ar for 6 h. The reaction mixture was concentrated under vacuum and the residue was purified by SGCC eluted with EtOAc/PE (1/1) to afford the title compound as yellow solid (2.5 g). LCMS (ESI): 300.4 [M+1]⁺.

### Step 4 1-(5-(4-cyclopropyl-6-methoxypyrimidin-5-yl)-1H-pyrazolo[4,3-d]pyrimidin-1-yl)ethan-1-one

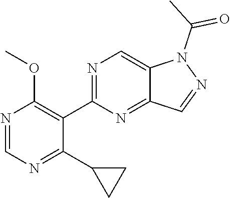 [SMILES: COc1ncnc(C2CC2)c1-c1ncc2c(cnn2C(C)=O)n1]

To a stirred solution of the product of Step 3 (12.0 g) and KOAc (3.9 g) in 1,4-dioxane (100 mL) were added acetic anhydride (14.7 g) and isoamyl nitrite (14.1 g) at r.t. and then the reaction mixture was stirred at 80° C. overnight. The reaction mixture was quenched with water and extracted with EtOAc (50 mL×3). The combined organic layers were dried over Na₂SO₄, filtered and concentrated under reduced pressure. The residue was purified by SGCC (0-50% EtOAc/PE) to afford the title compound (12.5 g) as yellow solid. LCMS (ESI): 311.3 [M+1]⁺.

### Step 5 5-(4-cyclopropyl-6-methoxypyrimidin-5-yl)-3-iodo-1H-pyrazolo[4,3-d]pyrimidine

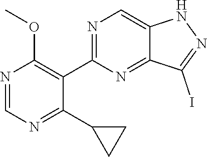 [SMILES: COc1ncnc(C2CC2)c1-c1ncc2[nH]nc(I)c2n1]

A solution of the product of Step 4 (12.0 g) in methanol (100 mL) was added Cs₂CO₃ (38.0 g) and the reaction mixture was stirred at 60° C. for 1 h. After being cooled to 25° C., N-iodosuccinimide (18.5 g) was added to the reaction mixture and the mixture was stirred at 25° C. for 1 h. The reaction solution was then concentrated and treated with NaHCO₃ solution (50 mL) and extracted with EtOAc (50 mL×3). The combined organic layer was concentrated. The residue was purified by SGCC (DCM/methanol: from 100:0 to 10:1) to afford the title compound as white solid (15.5 g). LCMS (ESI): 395.4 [M+1]⁺.

### Step 6 5-(4-cyclopropyl-6-methoxypyrimidin-5-yl)-3-iodo-1-methyl-1H-pyrazolo[4,3-d]pyrimidine

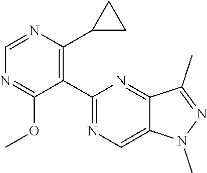 [SMILES: COc1ncnc(C2CC2)c1-c1ncc2c(n1)c(C)nn2C]

To a stirred solution of the product of Step 5 (0.7 g) and Cs₂CO₃ (1.8 g) in DMSO (10.0 mL), iodomethane (1.5 g) was added at 0° C. After stirring at 0° C. for 3 h, the reaction mixture was quenched with NaHCO₃ solution and extracted with EtOAc (50 mL×3). The combined organic layer was washed with water and concentrated. The residue was purified by SGCC (DCM/methanol: from 100:0 to 10:1) to afford the title compound as white solid (580 mg). LCMS (ESI): 409.3 [M+1]⁺.

### Step 7 3-(2-(benzyloxy)-4-(1-isopropyl-4-(trifluoromethyl)-1H-imidazol-2-yl)benzyl)-5-(4-cyclopropyl-6-methoxypyrimidin-5-yl)-1-methyl-1H-pyrazolo[4,3-d]pyrimidine

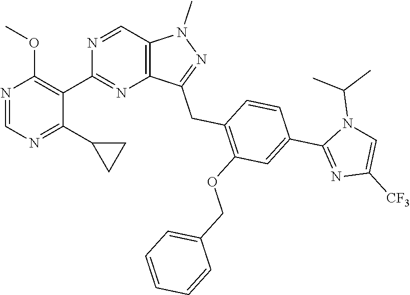 [SMILES: COc1ncnc(C2CC2)c1-c1ncc2c(n1)c(Cc1ccc(-c3nc(C(F)(F)F)cn3C(C)C)cc1OCc1ccccc1)nn2C]

To a stirred solution of the product of Step 5 in Example 16 (0.41 g) in DMSO (10.0 mL) was added sodium 3-methoxy-3-oxopropane-1-sulfinate (0.17 g) at 25° C. After stirring at 50° C. for 1 h, to the reaction mixture were added the product of the step 6 (0.3 g), di-tert-butyl(methyl)phosphonium tetrafluoroborate (0.11 g), Pd(OAc)₂ (52 mg) and K₂CO₃ (0.3 g) at 20° C. The reaction mixture was stirred at 100° C. for 3 h under Ar and then the reaction mixture was concentrated. The residue was partitioned between water (20 mL) and EtOAc (30 mL). The organic layer was washed with brine, dried over anhydrous Na₂SO₄ and concentrated under vacuum. The residue was purified by SGCC eluted with EtOAc/PE (0% to 100% EtOAc) to afford the title product as white solid (360 mg). LCMS (ESI): 655.3 [M+1]⁺.

### Step 8 2-((5-(4-cyclopropyl-6-methoxypyrimidin-5-yl)-1-methyl-1H-pyrazolo[4,3-d]pyrimidin-3-yl) methyl)-5-(1-isopropyl-4-(trifluoromethyl)-1H-imidazol-2-yl)phenol

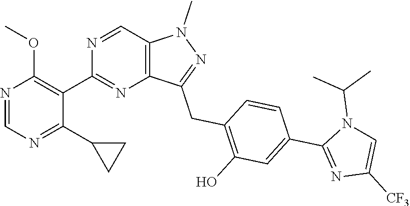 [SMILES: COc1ncnc(C2CC2)c1-c1ncc2c(n1)c(Cc1ccc(-c3nc(C(F)(F)F)cn3C(C)C)cc1O)nn2C]

The product of step 7 (0.26 g) was added to TFA (5 mL) and the mixture was stirred at 80° C. for 16 h. The reaction mixture was concentrated and the residue was poured into saturated NaHCO₃ solution (10.0 mL) and extracted with EtOAc (30 mL×3). The combined organic layer was washed with brine, dried over anhydrous Na₂SO₄ and concentrated under vacuum. The residue was purified using SGCC eluted with EtOAc/PE (0% to 100% EtOAc) to afford the title product as white solid (0.2 g). LCMS (ESI): 565.4 [M+1]⁺.

### Step 9 3-(2-(2-((tert-butyldimethylsilyl)oxy)ethoxy)-4-(1-isopropyl-4-(trifluoromethyl)-1H-imidazol-2-yl)benzyl)-5-(4-cyclopropyl-6-methoxypyrimidin-5-yl)-1-methyl-1H-pyrazolo[4,3-d]pyrimidine

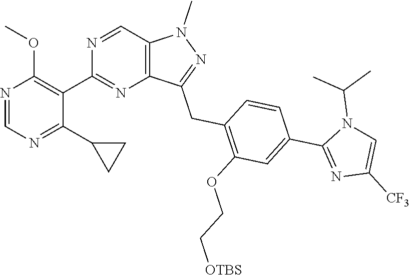 [SMILES: COc1ncnc(C2CC2)c1-c1ncc2c(n1)c(Cc1ccc(-c3nc(C(F)(F)F)cn3C(C)C)cc1OCCO[Si](C)(C)C(C)(C)C)nn2C]

To a solution of the product of step 8 (0.2 g) and K₂CO₃ (200 mg) in DMSO (5.0 mL) was added (2-bromoethoxy)(tert-butyl)dimethylsilane (850 mg). After stirring at 60° C. for 1 h, the reaction solution was poured into water and extracted with EtOAc (30 mL×3). The combined organic layer was washed with 50 mL saturated brine, dried over anhydrous Na₂SO₄ and concentrated. The residue was purified by SGCC (PE/EtOAc: from 100:0 to 5:1) to afford product as yellow oil (210 mg). LCMS (ESI): 723.3 [M+1]⁺.

### Step 10 1⁶-cyclopropyl-4⁴-(1-isopropyl-4-(trifluoromethyl)-1H-imidazol-2-yl)-2¹-methyl-2¹H-5,8-dioxa-2(5,3)-pyrazolo[4,3-d]pyrimidina-1(5,4)-pyrimidina-4(1,2)-benzenacyclooctaphane

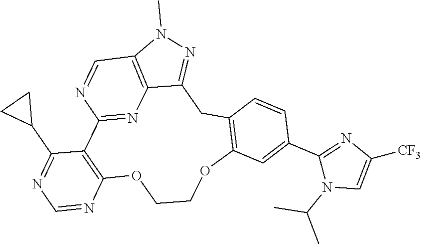

Starting with the product of Step 9, the title product was obtained as a white solid by following the same procedures as described in Step 13 and Step 14 of Example 3. ¹H NMR (500 MHz, CDCl₃) δ 9.13 (s, 1H), 8.73 (s, 1H), 7.67 (d, 1H), 7.40 (s, 1H), 7.06 (dd, 1H), 6.99 (d, 1H), 4.77-4.72 (m, 2H), 4.64-4.55 (m, 1H), 4.54 (s, 2H), 4.23-4.18 (m, 2H), 4.16 (s, 3H), 2.83-2.74 (m, 1H), 1.45 (d, 6H), 1.36-1.31 (m, 2H), 1.11-1.03 (m, 2H).

### Example 23

### 1⁶-cyclopropyl-4⁴-(1-isopropyl-4-(trifluoromethyl)-1H-imidazol-2-yl)-5-methyl-2¹H-9-oxa-5-aza-2(6,1)-pyrazolo[3,4-d]pyrimidina-1(5,4)-pyrimidina-4(1,2)-benzenacyclononaphane

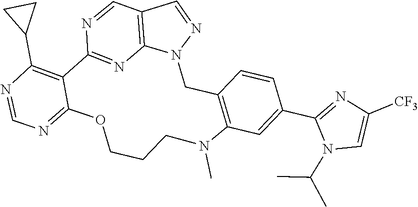 [SMILES: CC(C)n1cc(C(F)(F)F)nc1-c1ccc2c(c1)N(C)CCCOc1ncnc(C3CC3)c1-c1ncc3cnn(c3n1)C2]

### Step 1 (Z)-1⁶-cyclopropyl-4⁴-(1-isopropyl-4-(trifluoromethyl)-1H-imidazol-2-yl)-5-methyl-2⁴,2⁵-dihydro-2¹H-9-oxa-5-aza-2(6,1)-pyrazolo[3,4-d]pyrimidina-1(5,4)-pyrimidina-4(1,2)-benzenacyclononaphane

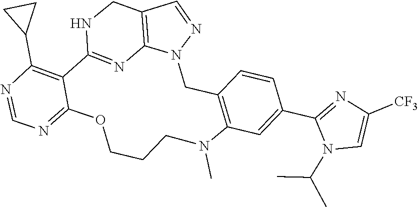 [SMILES: CC(C)n1cc(C(F)(F)F)nc1-c1ccc2c(c1)N(C)CCCOc1ncnc(C3CC3)c1C1=Nc3c(cnn3C2)CN1]

A solution of Example 8 (30 mg) and paraformaldehyde (30 mg) in 0.5 mL acetic acid was stirred at r.t. for 1 h. NaBH(OAc)₃ (100 mg) was then added. After stirring for additional 0.5 h, the mixture was adjusted to pH=8 with aqueous K₂CO₃ solution and extracted with EtOAc (30 mL×3). The combined organic layer was concentrated to obtain the crude product as yellow solid (30 mg). LCMS (ESI): 592.3 [M+1]⁺.

### Step 2 1⁶-cyclopropyl-4⁴-(1-isopropyl-4-(trifluoromethyl)-1H-imidazol-2-yl)-5-methyl-2¹H-9-oxa-5-aza-2(6,1)-pyrazolo[3,4-d]pyrimidina-1(5,4)-pyrimidina-4(1,2)-benzenacyclononaphane

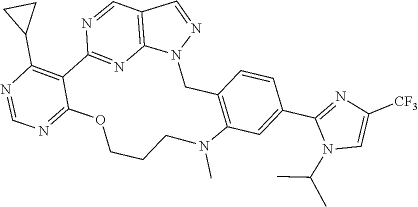 [SMILES: CC(C)n1cc(C(F)(F)F)nc1-c1ccc2c(c1)N(C)CCCOc1ncnc(C3CC3)c1-c1ncc3cnn(c3n1)C2]

To a solution of the product of step 1 (30 mg) in DCM (5 mL) were added 2,3-dichloro-5,6-dicyano-1,4-benzoquinone (50 mg) and stirred at r.t. for 0.5 h. The mixture was washed with saturated aqueous potassium bicarbonate solution (3×50 mL). The organic layer was concentrated and the residue was purified by SGCC (DCM/methanol: from 100:0 to 20:1) to obtain the product as white solid (21 mg). LCMS (ESI): 590.3 [M+1]⁺.

### Example 24

1⁶-cyclopropyl-4⁴-(1-isopropyl-4-(trifluoromethyl)-1H-imidazol-2-yl)-2⁷,2⁸-dihydro-8-oxa-5-aza-2(2,8)-pyrido[2,3-d]pyrimidina-1(5,4)-pyrimidina-4(1,2)-benzenacyclooctaphan-2⁷-one

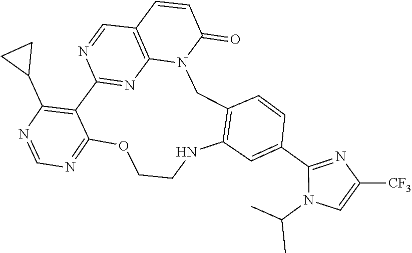 [SMILES: CC(C)n1cc(C(F)(F)F)nc1-c1ccc2c(c1)NCCOc1ncnc(C3CC3)c1-c1ncc3ccc(=O)n(c3n1)C2]

Using 2-((tert-butyldimethylsilyl)oxy)acetaldehyde as the starting material in Step 3 of Example 20, the title compound was prepared by the same procedures as described for Example 20. LCMS (ESI): 589.5 [M+1]⁺.

### Example 25

### 1⁶-cyclopropyl-4⁴-(1-isopropyl-4-(trifluoromethyl)-1H-imidazol-2-yl)-2⁷,2⁸-dihydro-8-oxa-2(2,8)-pyrido[2,3-d]pyrimidina-1(5,4)-pyrimidina-4(1,2)-benzenacyclooctaphan-2⁷-one

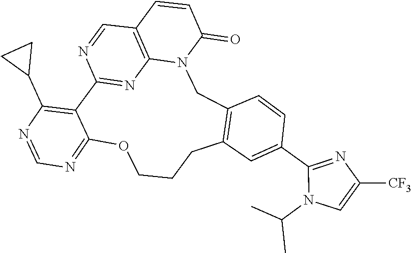 [SMILES: CC(C)n1cc(C(F)(F)F)nc1-c1ccc2c(c1)CCCOc1ncnc(C3CC3)c1-c1ncc3ccc(=O)n(c3n1)C2]

### Step 1 2-(3-bromo-4-(chloromethyl)phenyl)-1-isopropyl-4-(trifluoromethyl)-1H-imidazole

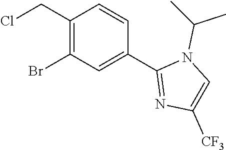 [SMILES: CC(C)n1cc(C(F)(F)F)nc1-c1ccc(CCl)c(Br)c1]

To a solution of the product of Step 3 in Example 9 (1.5 g) in DCM (50 mL) was added thionyl chloride (15 mL) at 0° C. After stirring at r.t. for 2 h, the mixture was then concentrated to give the product as yellow solid (1.8 g). LCMS (ESI): 381.2 [M+1]⁺.

### Step 2 8-(2-bromo-4-(1-isopropyl-4-(trifluoromethyl)-1H-imidazol-2-yl)benzyl)-2-(4-cyclopropyl-6-methoxypyrimidin-5-yl)pyrido[2,3-d]pyrimidin-7(8H)-one

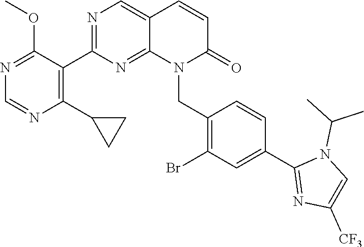 [SMILES: COc1ncnc(C2CC2)c1-c1ncc2ccc(=O)n(Cc3ccc(-c4nc(C(F)(F)F)cn4C(C)C)cc3Br)c2n1]

A mixture of the product of Step 1 (407 mg), Step 4 in Example 16 (350 mg) and Cs₂CO₃ (773 mg) in DMF (30 mL) was stirred at r.t. for 5 h. The mixture was then poured into water (50 mL) and extracted with EtOAc (50 mL×3). The combined organic layer was dried over anhydrous Na₂SO₄ and concentrated under vacuum. The residue was purified by SGCC eluted with EtOAc/PE (0% to 100% EtOAc) to afford the title product as white solid (560 mg). LCMS (ESI): 640.2 [M+1]⁺.

### Step 3 ethyl (E)-3-(2-((2-(4-cyclopropyl-6-methoxypyrimidin-5-yl)-7-oxopyrido[2,3-d]pyrimidin-8(7H)-yl)methyl)-5-(1-isopropyl-4-(trifluoromethyl)-1H-imidazol-2-yl)phenyl)acrylate

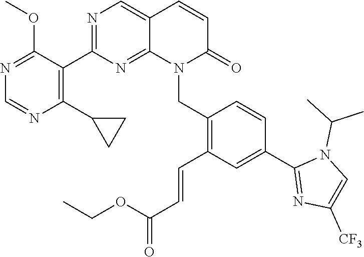 [SMILES: CCOC(=O)/C=C/c1cc(-c2nc(C(F)(F)F)cn2C(C)C)ccc1Cn1c(=O)ccc2cnc(-c3c(OC)ncnc3C3CC3)nc21]

A mixture of the product of Step 2 (50 mg), ethyl (E)-3-(4,4,5,5-tetramethyl-1,3,2-dioxaborolan-2-yl)acrylate (53 mg), CsF (36 mg), Pd₂(dba)₃ (14 mg) and X—PHOS (19 mg) in 1,4-dioxane (30 mL) was degassed under vacuum and the mixture was purged with Ar three times. The reaction mixture was stirred at 90° C. overnight. The reaction mixture was then concentrated under reduced pressure and the residue was purified by SGCC eluted with EtOAc/PE (0% to 100% EtOAc) to afford the title product as white solid (28 mg). LCMS (ESI): 660.3 [M+1]⁺.

### Step 4 ethyl 3-(2-((2-(4-cyclopropyl-6-methoxypyrimidin-5-yl)-7-oxopyrido[2,3-d]pyrimidin-8(7H)-yl) methyl)-5-(1-isopropyl-4-(trifluoromethyl)-1H-imidazol-2-yl)phenyl)propanoate

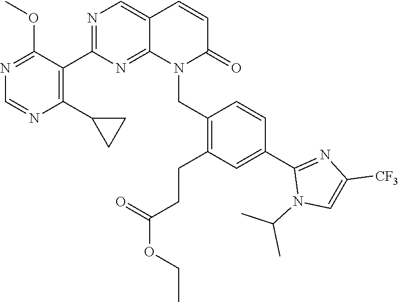 [SMILES: CCOC(=O)CCc1cc(-c2nc(C(F)(F)F)cn2C(C)C)ccc1Cn1c(=O)ccc2cnc(-c3c(OC)ncnc3C3CC3)nc21]

To a mixture of the product of Step 3 (800 mg) and Pd(OAc)₂ (3 mg) in DCM (30 mL) was added pinacolborane (171 mg). After stirring at rt for 4 h, the reaction mixture was poured into 100 mL ice-water, and extracted with DCM (50×3 mL). The combined organic layer was washed with brine, dried over anhydrous Na₂SO₄ and then concentrated to afford the title compound as yellow solid (654 mg). LCMS (ESI): 662.2 [M+1]⁺.

### Step 5 2-(4-cyclopropyl-6-methoxypyrimidin-5-yl)-8-(2-(3-hydroxypropyl)-4-(1-isopropyl-4-(trifluoromethyl)-1H-imidazol-2-yl)benzyl)pyrido[2,3-d]pyrimidin-7(8H)-one

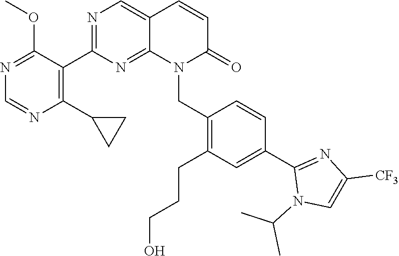 [SMILES: COc1ncnc(C2CC2)c1-c1ncc2ccc(=O)n(Cc3ccc(-c4nc(C(F)(F)F)cn4C(C)C)cc3CCCO)c2n1]

To a mixture of the product of Step 4 (800 mg) in DCM (30 mL) (1 ml) was added DIBAL-H (0.2 mL) at 0° C. After stirring for 30 min, the mixture was adjusted to pH=5 with HCl (1 N) and extracted with EtOAc (50×3 mL). The combined organic layer was washed with brine, dried over anhydrous Na₂SO₄ and then concentrated to afford the title compound as yellow solid (35 mg). LCMS (ESI): 620.3 [M+1]⁺.

### Step 6 1⁶-cyclopropyl-4⁴-(1-isopropyl-4-(trifluoromethyl)-1H-imidazol-2-yl)-2⁷,2⁸-dihydro-8-oxa-2(2,8)-pyrido[2,3-d]pyrimidina-1(5,4)-pyrimidina-4(1,2)-benzenacyclooctaphan-2⁷-one

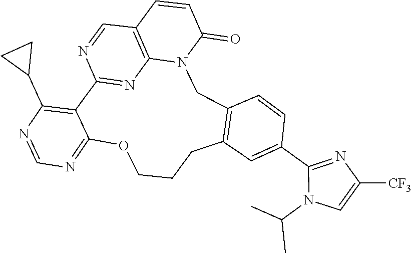 [SMILES: CC(C)n1cc(C(F)(F)F)nc1-c1ccc2c(c1)CCCOc1ncnc(C3CC3)c1-c1ncc3ccc(=O)n(c3n1)C2]

Starting with the product of Step 5, the title product was obtained as a white solid by following the same procedures as described in Step 13 and Step 14 of Example 3. LCMS (ES+): 588.4 [M+1]⁺.

### Example 26

### 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1,4(1,2)-dibenzenacyclononaphane

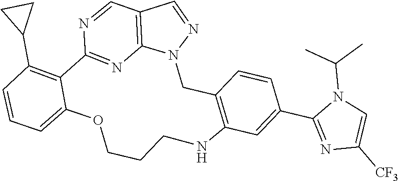 [SMILES: CC(C)n1cc(C(F)(F)F)nc1-c1ccc2c(c1)NCCCOc1cccc(C3CC3)c1-c1ncc3cnn(c3n1)C2]

### Step 1 6-chloro-1-(4-(1-isopropyl-4-(trifluoromethyl)-1H-imidazol-2-yl)-2-nitrobenzyl)-1H-pyrazolo [3,4-d]pyrimidine

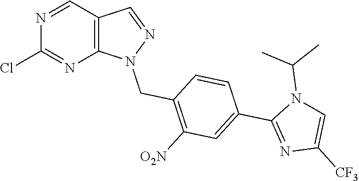 [SMILES: CC(C)n1cc(C(F)(F)F)nc1-c1ccc(Cn2ncc3cnc(Cl)nc32)c([N+](=O)[O-])c1]

A mixture of the product of Step 4 in Example 8 (2.5 g), 6-chloro-1H-pyrazolo[3,4-d]pyrimidine (0.8 g) and K₂CO₃ (1.4 g) in DMF (20 mL) was stirred for 1 h at 60° C. The reaction mixture was then treated with ammonium chloride (1N, 100 mL) and extracted with EtOAc (3×50 mL). The combined organic layer was dried over anhydrous Na₂SO₄ and concentrated. The residue was purified by SGCC (PE/EtOAc: from 100:0 to 50:50) to afford the title compound as yellow solid (1.25 g). LCMS (ESI): 466.3[M+1]⁺.

### Step 2 6-(2-chloro-6-methoxyphenyl)-1-(4-(1-isopropyl-4-(trifluoromethyl)-1H-imidazol-2-yl)-2-nitrobenzyl)-1H-pyrazolo[3,4-d]pyrimidine

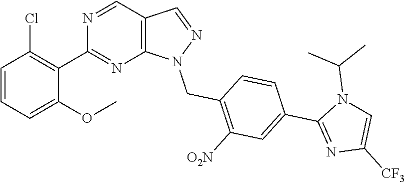 [SMILES: COc1cccc(Cl)c1-c1ncc2cnn(Cc3ccc(-c4nc(C(F)(F)F)cn4C(C)C)cc3[N+](=O)[O-])c2n1]

To a mixture of the product of Step 1 (900 mg) and (2-chloro-6-methoxyphenyl)boronic acid (1.2 g) in 1,4-dioxane/water (20/2 mL) at r.t. were added Pd₂(dba)₃ (180 mg), 2-(dicyclohexylphosphino)-2,4,6-triisopropylbiphenyl (191 mg) and CsF (900 mg). After stirring at 90° C. for 12 h, the mixture was poured into 50 mL ice-water and extracted with EtOAc (3×50 mL). The combined organic layer was washed with 50 mL of saturated brine, dried over anhydrous Na₂SO₄ and concentrated. The residue was purified by SGCC (PE/EtOAc: from 100:0 to 50:50) to afford the title compound as pale yellow solid (420 mg). LCMS (ESI): 572.4 [M+1]⁺.

### Step 3 2-((6-(2-chloro-6-methoxyphenyl)-1H-pyrazolo[3,4-d]pyrimidin-1-yl)methyl)-5-(1-isopropyl-4-(trifluoromethyl)-1H-imidazol-2-yl)aniline

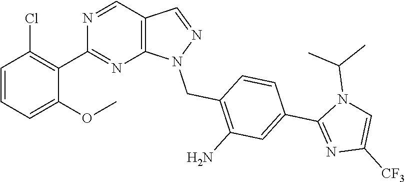 [SMILES: COc1cccc(Cl)c1-c1ncc2cnn(Cc3ccc(-c4nc(C(F)(F)F)cn4C(C)C)cc3N)c2n1]

To a mixture of the product of Step 2 (400 mg) and Zn powder (450 mg) in methanol (15 mL) was added ammonium chloride (430 mg). The reaction mixture was stirred at r.t. for 18 h and then solid was removed by filtration. The filtrate was concentrated and the residue was purified by SGCC (PE/EtOAc: from 100:0 to 50:50) to afford the product as white solid (390 mg). LCMS (ESI): 542.4 [M+1]⁺.

### Step 4 N-(3-((tert-butyldimethylsilyl)oxy)propyl)-2-((6-(2-chloro-6-methoxyphenyl)-1H-pyrazolo[3,4-d]pyrimidin-1-yl)methyl)-5-(1-isopropyl-4-(trifluoromethyl)-1H-imidazol-2-yl)aniline

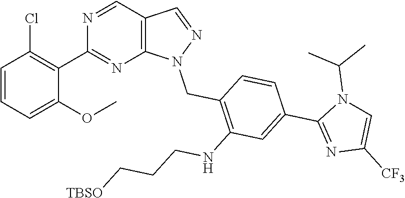 [SMILES: COc1cccc(Cl)c1-c1ncc2cnn(Cc3ccc(-c4nc(C(F)(F)F)cn4C(C)C)cc3NCCCO[Si](C)(C)C(C)(C)C)c2n1]

To a solution of the product of Step 3 (0.3 g) and 3-((tert-butyldimethylsilyl)oxy)propanal (0.16 g) in DCM (4 mL) was added NaBH(OAc)₃ (0.46 g) and the reaction mixture was stirred at r.t. for 1 h. The reaction mixture was then concentrated and the residue was purified by SGCC (DCM/methanol: from 100:0 to 50:50) to afford the product as white solid (0.4 g). LCMS (ESI): 714.6 [M+1]⁺.

### Step 5 3-chloro-2-(1-(2-((3-hydroxypropyl)amino)-4-(1-isopropyl-4-(trifluoromethyl)-1H-imidazol-2-yl)benzyl)-1H-pyrazolo[3,4-d]pyrimidin-6-yl)phenol

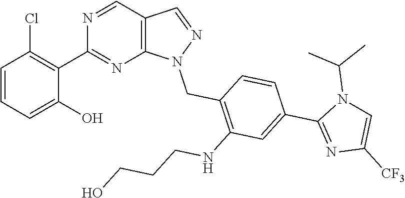 [SMILES: CC(C)n1cc(C(F)(F)F)nc1-c1ccc(Cn2ncc3cnc(-c4c(O)cccc4Cl)nc32)c(NCCCO)c1]

To a solution of product of Step 4 (0.4 g) in DCM (10 mL) was added boron tribromide (0.5 mL, 1 M in DCM) at −78° C. The reaction mixture was stirred at r.t. for 3 h and then treated with methanol (1.0 mL) and NaHCO₃ (2 N, 60 mL), followed by extraction with EtOAc (3×20 mL). The organic layer was dried over anhydrous Na₂SO₄ and concentrated in vacuum to afford the title compound as white solid (240 mg). LCMS (ESI): 586.5 [M+1]⁺.

### Step 6 1⁶-chloro-4⁴-(1-isopropyl-4-(trifluoromethyl)-1H-imidazol-2-yl)-2¹H-9-oxa-5-aza-2(6,1)-pyrazolo[3,4-d]pyrimidina-1,4(1,2)-dibenzenacyclononaphane

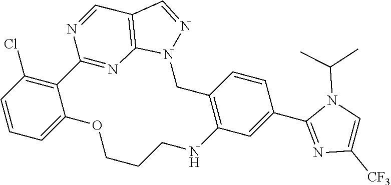 [SMILES: CC(C)n1cc(C(F)(F)F)nc1-c1ccc2c(c1)NCCCOc1cccc(Cl)c1-c1ncc3cnn(c3n1)C2]

Starting with the product of Step 5, the title product was obtained as a white solid by following the same procedures as described in Step 14 of Example 3. LCMS (ESI): 568.4 [M+1]⁺.

### Step 7 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1,4(1,2)-dibenzenacyclononaphane

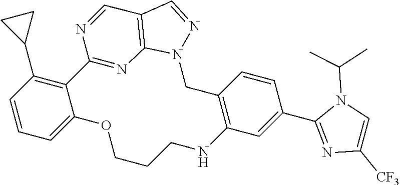 [SMILES: CC(C)n1cc(C(F)(F)F)nc1-c1ccc2c(c1)NCCCOc1cccc(C3CC3)c1-c1ncc3cnn(c3n1)C2]

Substituting isopropylzinc bromide with cyclopropylzinc bromide, the title product was prepared by using the same procedures as described in Example 7. LCMS (ESI): 574.5 [M+1]⁺.

### Example 27

### 1⁶-cyclopropyl-4⁴-(1-isopropyl-4-(trifluoromethyl)-1H-imidazol-2-yl)-2¹H-8-oxa-5-thia-2(6,1)-pyrazolo[3,4-d]pyrimidina-1(5,4)-pyrimidina-4(1,2)-benzenacyclooctaphane

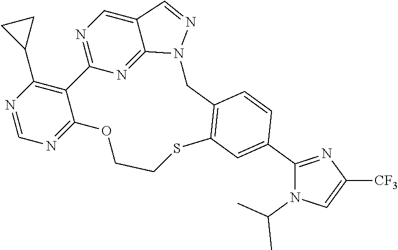 [SMILES: CC(C)n1cc(C(F)(F)F)nc1-c1ccc2c(c1)SCCOc1ncnc(C3CC3)c1-c1ncc3cnn(c3n1)C2]

### Step 1 2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyltrifluoromethanesulfonate

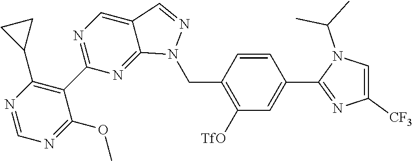 [SMILES: COc1ncnc(C2CC2)c1-c1ncc2cnn(Cc3ccc(-c4nc(C(F)(F)F)cn4C(C)C)cc3OS(=O)(=O)C(F)(F)F)c2n1]

To a solution of the product of Step 11 (300 mg) in Example 3 and pyridine (86 mg) in DCM (5 mL) was added trifluoromethanesulfonic anhydride (200 mg) at 0° C. After being stirred at r.t. for 1 h, the reaction solution was concentrated and the residue was purified by SGCC (PE/EtOAc: from 100:0 to 20:80) to afford the title product (334 mg) as white solid. LCMS (ESI): 683.2 [M+1]⁺.

### Step 2 ethyl 2-((2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl)thio)acetate

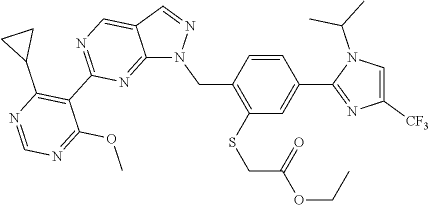 [SMILES: CCOC(=O)CSc1cc(-c2nc(C(F)(F)F)cn2C(C)C)ccc1Cn1ncc2cnc(-c3c(OC)ncnc3C3CC3)nc21]

To a mixture of the product of Step 1 (300 mg) and ethyl 2-mercaptoacetate (98 mg) in 1,4-dioxane (5 mL) at r.t. were added Pd₂(dba)₃ (154 mg), 4,5-bis(diphenylphosphino)-9,9-dimethylxanthene (315 mg) and i-Pr₂NEt (141 mg). The reaction mixture was stirred at 100° C. for 18 h and then poured into 20 mL ice-water and extracted with EtOAc (3×20 mL). The combined organic layer was washed with saturated brine (30 mL), dried over anhydrous Na₂SO₄ and concentrated. The residue was purified by SGCC (PE/EtOAc: from 100:0 to 10:1) to afford the title compound as yellow solid (260 mg). LCMS (ESI): 653.5 [M+1]⁺.

### Step 3 2-((2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl)thio)ethan-1-ol

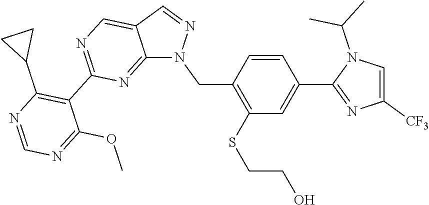 [SMILES: COc1ncnc(C2CC2)c1-c1ncc2cnn(Cc3ccc(-c4nc(C(F)(F)F)cn4C(C)C)cc3SCCO)c2n1]

To a solution of the product of Step 2 (50 mg) in anhydrous DCM (2 mL) was added dropwise DIBAL-H (0.2 mL, 1M in hexane) at 0° C. The reaction solution was stirred at 0° C. for 1 h and then quenched by Na₂SO₄ decahydrate (1.0 g). After stirring at r.t. for 30 min, the resulting mixture was filtered through a celite pad and washed with a mixture of methanol and DCM (1/5, 30 mL). The filtrate was concentrated under vacuum to afford the title product as yellow solid (27 mg). LCMS (ESI): 610.2 [M+1]⁺.

### Step 4 1⁶-cyclopropyl-4⁴-(1-isopropyl-4-(trifluoromethyl)-1H-imidazol-2-yl)-2¹H-8-oxa-5-thia-2(6,1)-pyrazolo[3,4-d]pyrimidina-1(5,4)-pyrimidina-4(1,2)-benzenacyclooctaphane

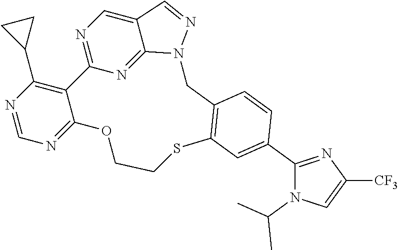 [SMILES: CC(C)n1cc(C(F)(F)F)nc1-c1ccc2c(c1)SCCOc1ncnc(C3CC3)c1-c1ncc3cnn(c3n1)C2]

Starting with the product of Step 3, the title product was obtained as a white solid by following the same procedures as described in Step 13 and Step 14 of Example 3. LCMS (ESI): 579.5 [M+1]⁺.

### Example 28

### 1⁶cyclopropyl-4⁴-(1-isopropyl-4-(trifluoromethyl)-1H-imidazol-2-yl)-2¹H-6,9-dioxa-2(6,1)-pyrazolo[3,4-d]pyrimidina-1(5,4)-pyrimidina-4(1,2)-benzenacyclononaphane

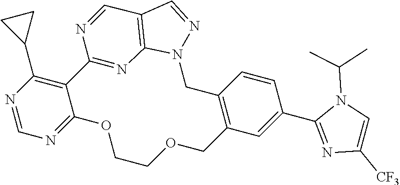 [SMILES: CC(C)n1cc(C(F)(F)F)nc1-c1ccc2c(c1)COCCOc1ncnc(C3CC3)c1-c1ncc3cnn(c3n1)C2]

### Step 1 methyl 2-((6-(4-cyclopropyl-6-methoxypyrimidin-5-yl)-1H-pyrazolo [3,4-d]pyrimidin-1-yl)methyl)-5-(1-isopropyl-4-(trifluoromethyl)-1H-imidazol-2-yl)benzoate

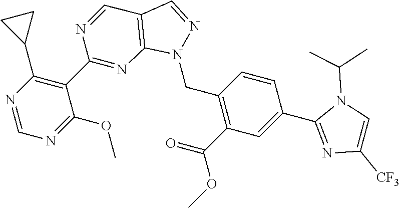 [SMILES: COC(=O)c1cc(-c2nc(C(F)(F)F)cn2C(C)C)ccc1Cn1ncc2cnc(-c3c(OC)ncnc3C3CC3)nc21]

A mixture of the product of Step 1 in Example 27 (1.6 g), Pd(OAc)₂ (29 mg) and (9,9-dimethyl-9H-xanthene-4,5-diyl)bis(diphenylphosphine) (75 mg) in methanol (40 mL) was degassed under vacuum and refilled with CO for three times. The resulting mixture was stirred at 70° C. overnight under carbon monoxide atmosphere. The reaction mixture was then cooled to r.t. and filtered through celite pad. The celite pad was washed with methanol (50 mL). The filtrate was concentrated under vacuum and the residue was dissolved in EtOAc (20 mL) and then filtered through celite pad which was washed with EtOAc (30 mL). The filtrate was concentrated under vacuum to afford the title product as yellow solid (1.0 g). LCMS (ESI): 593.5 [M+1]⁺.

### Step 2 (2-((6-(4-cyclopropyl-6-methoxypyrimidin-5-yl)-1H-pyrazolo [3,4-d]pyrimidin-1-yl)methyl)-5-(1-isopropyl-4-(trifluoromethyl)-1H-imidazol-2-yl)phenyl)methanol

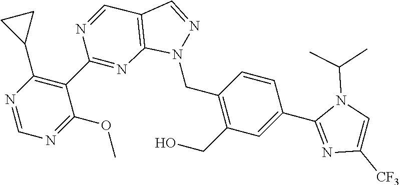 [SMILES: COc1ncnc(C2CC2)c1-c1ncc2cnn(Cc3ccc(-c4nc(C(F)(F)F)cn4C(C)C)cc3CO)c2n1]

To a solution of the product of Step 1 (600 mg) in DCM (10 mL) was added dropwise DIBAL-H (2.4 mL, 1M in hexane) at 0° C. After stirring at 0° C. for 1 h, the reaction mixture was treated with Na₂SO₄ decahydrate (6.0 g) and stirred at rt for 30 min. The resulting mixture was filtered through a celite pad which was washed with methanol/DCM (1/5, 200 mL). The filtrate was treated with DIAD (0.5 mL) and then concentrated under vacuum. The residue was purified by SGCC (PE/EtOAc: from 100:0 to 20:80) to afford the title product as yellow solid (400 mg, crude). LCMS (ESI): 565.6 [M+1]⁺.

### Step 3 1-(2-(chloromethyl)-4-(1-isopropyl-4-(trifluoromethyl)-1H-imidazol-2-yl) benzyl)-6-(4-cyclopropyl-6-methoxypyrimidin-5-yl)-1H-pyrazolo[3,4-d]pyrimidine

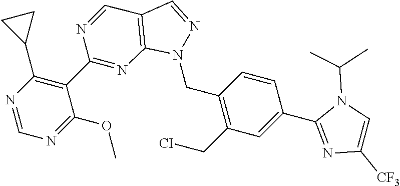

A solution of the product of Step 2 (400 mg) in DCM/thionyl chloride (10 mL, 10/1) was stirred at rt for 30 min. The reaction mixture was quenched by aqueous solution of NaHCO₃ at 0° C. and extracted with DCM (20 mL×2). The combined organic layer was dried over anhydrous Na₂SO₄ and concentrated under vacuum to afford the title product (300 mg, crude) as brown solid. LCMS (ESI): 582.3 [M+1]⁺.

### Step 4 1-(2-((2-((tert-butyldimethylsilyl)oxy)ethoxy)methyl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

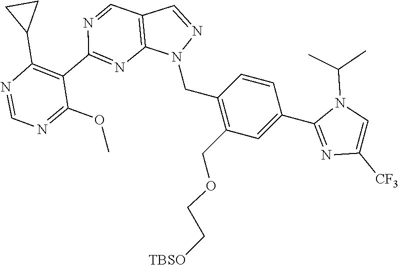 [SMILES: COc1ncnc(C2CC2)c1-c1ncc2cnn(Cc3ccc(-c4nc(C(F)(F)F)cn4C(C)C)cc3COCCO[Si](C)(C)C(C)(C)C)c2n1]

To a stirred mixture of 2-((tert-butyldimethylsilyl)oxy)ethan-1-ol (532 mg) and NaH (110 mg) in THE (2.0 mL) was added a mixture of the product of step 3 (160 mg) and KI (137 mg) in DMF (2.0 mL) at rt. After stirring at rt for 1 h, the reaction mixture was quenched by aq. ammonium chloride solution (10 mL) and extracted with EtOAc (20 mL×2). The combined organic layer was washed with brine (20 mL), dried over anhydrous Na₂SO₄ and concentrated under vacuum. The residue was purified by SGCC (PE/EtOAc: from 100:0 to 20:80) to afford the title product as yellow solid (100 mg). LCMS (ESI): 723.6 [M+1]⁺.

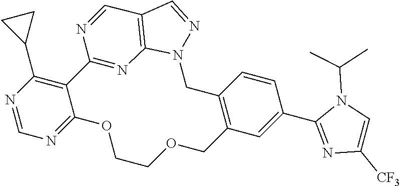 [SMILES: CC(C)n1cc(C(F)(F)F)nc1-c1ccc2c(c1)COCCOc1ncnc(C3CC3)c1-c1ncc3cnn(c3n1)C2]

Starting with the product of Step 4 (100 mg), the title product was obtained as a white solid (2 mg) by following the same procedures as described in Step 13 and 14 of Example 3. LCMS (ESI): 577.6 [M+1]⁺.

### Example 29

### 1⁶-cyclopropyl-44-(1-isopropyl-4-(trifluoromethyl)-1H-imidazol-2-yl)-2¹H-5,8-dioxa-2(6,1)-pyrazolo[3,4-d]pyrimidina-1(5,4)-pyrimidina-4(1,3)-benzenacyclooctaphane

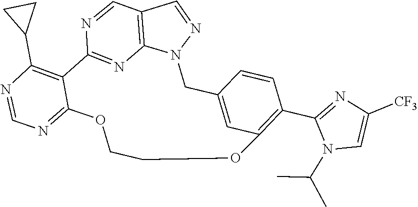 [SMILES: CC(C)n1cc(C(F)(F)F)nc1-c1ccc2cc1OCCOc1ncnc(C3CC3)c1-c1ncc3cnn(c3n1)C2]

### Step 1 2-(4-bromo-2-((4-methoxybenzyl)oxy)phenyl)-1-isopropyl-4-(trifluoromethyl)-1H-imidazole

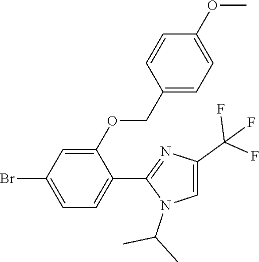 [SMILES: COc1ccc(COc2cc(Br)ccc2-c2nc(C(F)(F)F)cn2C(C)C)cc1]

Starting with 4-bromo-2-((4-methoxybenzyl)oxy)benzaldehyde (14 g), the title product was obtained as yellow solid (10 g, crude) by following the same procedures as described in Step 1 and Step 2 of Example 3. LCMS (ESI): 427.3 [M+1]⁺.

### Step 2 methyl 4-(1-isopropyl-4-(trifluoromethyl)-1H-imidazol-2-yl)-3-((4-methoxybenzyl)oxy) benzoate

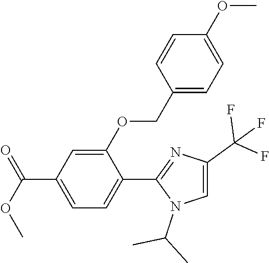 [SMILES: COC(=O)c1ccc(-c2nc(C(F)(F)F)cn2C(C)C)c(OCc2ccc(OC)cc2)c1]

Starting with the product of Step 1 (10 g), the title product was obtained as brown solid (4.0 g, crude) by following the same procedures as described in Step 1 of Example 28. LCMS (ESI): 449.3 [M+1]⁺.

### Step 3 (4-(1-isopropyl-4-(trifluoromethyl)-1H-imidazol-2-yl)-3-((4-methoxybenzyl)oxy)phenyl) methanol

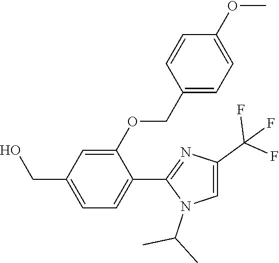 [SMILES: COc1ccc(COc2cc(CO)ccc2-c2nc(C(F)(F)F)cn2C(C)C)cc1]

Starting with the product of Step 2 (4.0 g), the title product was obtained as a yellow solid (2.4 g) by following the same procedures as described in Step 6 of Example 3. LCMS (ESI): 421.4 [M+1]⁺.

### Step 4 1-(3-(2-((tert-butyldimethylsilyl)oxy)ethoxy)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

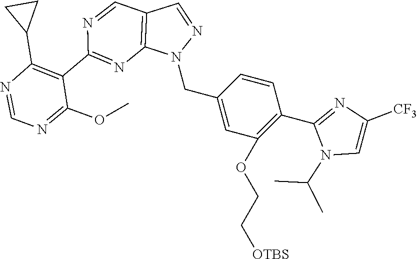 [SMILES: COc1ncnc(C2CC2)c1-c1ncc2cnn(Cc3ccc(-c4nc(C(F)(F)F)cn4C(C)C)c(OCCO[Si](C)(C)C(C)(C)C)c3)c2n1]

Starting with the product of Step 9 in Examples 3, the product of Step 3 and 2-((tert-butyldimethylsilyl)oxy)ethanol, the title product was obtained as yellow solid by following the same procedures as described in Step 10, Step 11 and Step 12 of Example 3. LCMS (ESI): 709.7 [M+1]⁺.

### Step 5 2-(5-((6-(4-cyclopropyl-6-methoxypyrimidin-5-yl)-1H-pyrazolo [3,4-d]pyrimidin-1-yl)methyl)-2-(1-isopropyl-4-(trifluoromethyl)-1H-imidazol-2-yl)phenoxy)ethan-1-ol

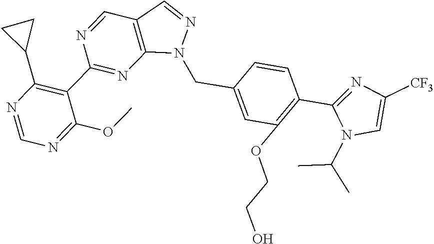 [SMILES: COc1ncnc(C2CC2)c1-c1ncc2cnn(Cc3ccc(-c4nc(C(F)(F)F)cn4C(C)C)c(OCCO)c3)c2n1]

To a solution of the product of Step 4 (500 mg) in 1,4-dioxane (4 mL) was added HCl (2 mL, 3M in H₂O) at rt. After stirring at rt for 1 h, the reaction mixture was diluted by EtOAc (30 mL) and washed with saturated aq. NaHCO₃ (20 mL), brine (20 mL). The organic layer was dried over anhydrous Na₂SO₄ and then concentrated under vacuum. The residue was purified by SGCC (PE/EtOAc: from 100:0 to 0:100) to afford the title product as pale yellow solid (340 mg). LCMS (ESI): 595.6 [M+1]⁺.

### Step 6 1⁶-cyclopropyl-44-(1-isopropyl-4-(trifluoromethyl)-1H-imidazol-2-yl)-2¹H-5,8-dioxa-2(6,1)-pyrazolo[3,4-d]pyrimidina-1(5,4)-pyrimidina-4(1,3)-benzenacyclooctaphane

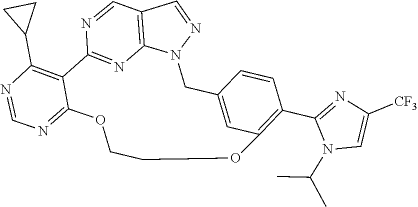 [SMILES: CC(C)n1cc(C(F)(F)F)nc1-c1ccc2cc1OCCOc1ncnc(C3CC3)c1-c1ncc3cnn(c3n1)C2]

To a solution of the product of Step 5 (100 mg) in dry-1,4-dioxane (10 mL) was added NaH (202 mg, 5.01 mmol) at room temperature. After stirring at 90° C. for 1 h, the reaction mixture was cooled to rt and quenched by AcOH (1.0 mL). The mixture was then washed with saturated aq. NaHCO₃ (20 mL), brine (20 mL). The organic layer was dried over anhydrous Na₂SO₄ and then concentrated under vacuum. The residue was purified by SGCC (PE/EtOAc: from 100:0 to 20:80) to afford the title product as white solid (34 mg). LCMS (ESI): 563.6 [M+1]⁺.

### Example 30

### (Z)-1⁶-cyclopropyl-4⁴-(1-cyclopropyl-4-(trifluoromethyl)-1H-imidazol-2-yl)-8-oxa-2(2,7)-pyrrolo[2,1-f][1,2,4]triazina-1(5,4)-pyrimidina-4(1,2)-benzenacyclooctaphane

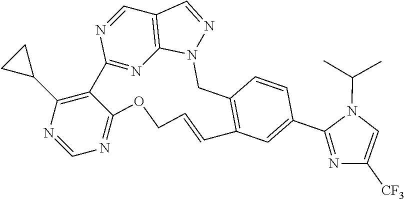 [SMILES: CC(C)n1cc(C(F)(F)F)nc1-c1ccc2c(c1)/C=C/COc1ncnc(C3CC3)c1-c1ncc3cnn(c3n1)C2]

### Step 1 (E)-1-(2-(3-((tert-butyldimethylsilyl)oxy)prop-1-en-1-yl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

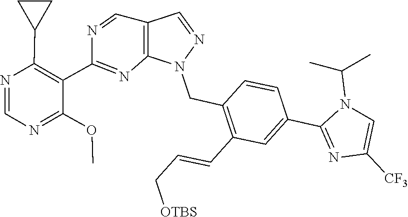 [SMILES: COc1ncnc(C2CC2)c1-c1ncc2cnn(Cc3ccc(-c4nc(C(F)(F)F)cn4C(C)C)cc3/C=C/CO[Si](C)(C)C(C)(C)C)c2n1]

To a mixture of the product of Step 1 in Example 27 (200 mg) and ethyl (E)-tert-butyldimethyl((3-(4,4,5,5-tetramethyl-1,3,2-dioxaborolan-2-yl)allyl)oxy)silane (1.25 g) in 1,4-dioxane/water (20/2 mL) were added Pd₂(dba)₃ (67.0 mg), 2-(dicyclohexylphosphino)-2,4,6-triisopropylbiphenyl (70.0 mg) and CsF (170 mg). After stirring at 100° C. under nitrogen for 18 h, the reaction mixture was poured into 100 mL ice-water and extracted with EtOAc (3×20 mL). The combined organic layer was washed with brine (20 mL), dried over anhydrous Na₂SO₄ and concentrated. The residue was purified by SGCC (PE/EtOAc: from 100:0 to 10:1) to afford the title compound as yellow solid (200 mg). LCMS (ESI): 705.3 [M+1]⁺.

### Step 2 (E)-1⁶-cyclopropyl-4⁴-(1-isopropyl-4-(trifluoromethyl)-1H-imidazol-2-yl)-2¹H-8-oxa-2(6,1)-pyrazolo[3,4-d]pyrimidina-1(5,4)-pyrimidina-4(1,2)-benzenacyclooctaphan-5-ene

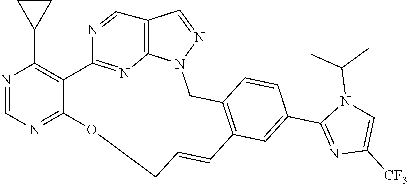 [SMILES: CC(C)n1cc(C(F)(F)F)nc1-c1ccc2c(c1)/C=C/COc1ncnc(C3CC3)c1-c1ncc3cnn(c3n1)C2]

Starting with the product of Step 1, the title product was obtained as a white solid by following the same procedures as described in Step 13 and Step 14 of Example 3. LCMS (ESI): 559.2 [M+1]⁺.

### Example 31

### (Z)-1⁶-cyclopropyl-4⁴-(1-isopropyl-4-(trifluoromethyl)-1H-imidazol-2-yl)-5,8-dioxa-2(2,7)-pyrrolo[2,1-f][1,2,4]triazina-1(5,4)-pyrimidina-4(1,2)-benzenacyclooctaphane

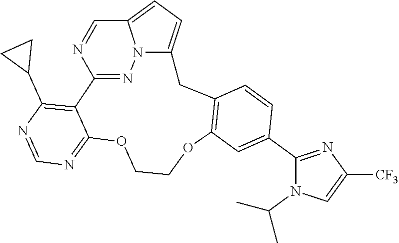 [SMILES: CC(C)n1cc(C(F)(F)F)nc1-c1ccc2c(c1)OCCOc1ncnc(C3CC3)c1-c1ncc3ccc(n3n1)C2]

Substituting the product of Step 2 in Example 21 with the product of Step 6 in Example 3, the title product was prepared under the same conditions as described in Example 21. LCMS (ESI): 562.6 [M+1]⁺. ¹H NMR (500 MHz, CDCl₃) δ 8.98 (s, 1H), 8.71 (s, 1H), 7.58 (d, 1H), 7.42 (s, 1H), 7.08 (d, 1H), 7.02 (s, 1H), 6.96 (d, 1H), 6.88 (d, 1H), 4.70 (t, 2H), 4.66-4.51 (m, 3H), 4.27 (t, 2H), 2.63-2.55 (m, 1H), 1.46 (d, 6H), 1.36-1.30 (m, 2H), 1.12-1.05 (m, 2H).

### Example 32

### (Z)-1⁶-cyclopropyl-4⁴-(1-cyclopropyl-4-(trifluoromethyl)-1H-imidazol-2-yl)-8-oxa-2(2,7)-pyrrolo[2,1-f][1,2,4]triazina-1(5,4)-pyrimidina-4(1,2)-benzenacyclooctaphane

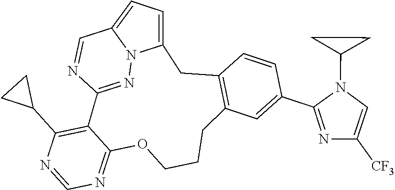 [SMILES: FC(F)(F)c1cn(C2CC2)c(-c2ccc3c(c2)CCCOc2ncnc(C4CC4)c2-c2ncc4ccc(n4n2)C3)n1]

### Step 1 5-(1-cyclopropyl-4-(trifluoromethyl)-1H-imidazol-2-yl)-2-((2-(4-cyclopropyl-6-methoxypyrimidin-5-yl)pyrrolo[2,1-f][1,2,4]triazin-7-yl)methyl)phenyltrifluoromethanesulfonate

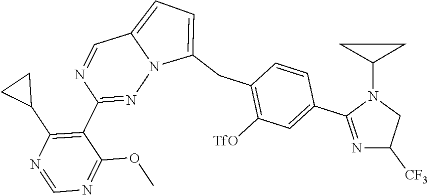 [SMILES: COc1ncnc(C2CC2)c1-c1ncc2ccc(Cc3ccc(C4=NC(C(F)(F)F)CN4C4CC4)cc3OS(=O)(=O)C(F)(F)F)n2n1]

To a solution of product of Step 8 in Example 21 (0.25 g) and pyridine (0.26 g) in DCM (6 mL) was added trifluoromethanesulfonic anhydride (1.0 mL) at 0° C. After being stirred at r.t. for 2 h, the mixture was then concentrated to give the crude product as yellow solid (0.24 g). LCMS (ESI): 680.2 [M+1]⁺.

### Step 2 (E)-7-(2-(3-((tert-butyldimethylsilyl)oxy)prop-1-en-1-yl)-4-(1-cyclopropyl-4-(trifluoromethyl)-1H-imidazol-2-yl)benzyl)-2-(4-cyclopropyl-6-methoxypyrimidin-5-yl)pyrrolo[2,1-f][1,2,4]triazine

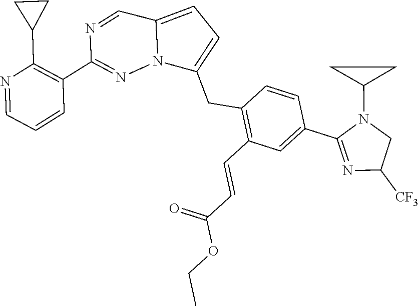 [SMILES: CCOC(=O)/C=C/c1cc(C2=NC(C(F)(F)F)CN2C2CC2)ccc1Cc1ccc2cnc(-c3cccnc3C3CC3)nn12]

To a mixture of the product of Step 1 (5.0 g) and ethyl (E)-3-(4,4,5,5-tetramethyl-1,3,2-dioxaborolan-2-yl)acrylate (6.8 g) in 1,4-dioxane/water (50/5 mL) at r.t. were added Pd₂(dba)₃ (1.6 g), 2-(dicyclohexylphosphino)-2,4,6-triisopropylbiphenyl (1.68 g) and CsF (5.3 g). After stirring at 100° C. under nitrogen atmosphere for 18 h, the reaction mixture was poured into 100 mL ice-water and extracted with EtOAc (3×50 mL). The combined organic layer was washed with brine (50 mL), dried over anhydrous Na₂SO₄ and concentrated. The residue was purified by SGCC (PE/EtOAc: from 100:0 to 10:1) to afford the title compound as yellow solid (6.0 g). LCMS (ESI): 630.2 [M+1]⁺.

### Step 3 (Z)-1⁶-cyclopropyl-4⁴-(1-cyclopropyl-4-(trifluoromethyl)-1H-imidazol-2-yl)-8-oxa-2(2,7)-pyrrolo[2,1-f][1,2,4]triazina-1(5,4)-pyrimidina-4(1,2)-benzenacyclooctaphane

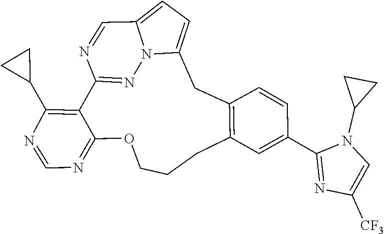 [SMILES: FC(F)(F)c1cn(C2CC2)c(-c2ccc3c(c2)CCCOc2ncnc(C4CC4)c2-c2ncc4ccc(n4n2)C3)n1]

Starting with the product of Step 2, the title product was obtained as white solid by following the same procedures as described in Step 6, Step 7 and Step 8 of Example 9. LCMS (ESI): 558.2 [M+1]⁺.

### Example 33

### (Z)-1⁶-cyclopropyl-4⁴-(1-isopropyl-4-(trifluoromethyl)-1H-imidazol-2-yl)-9-oxa-5-aza-2(2,7)-pyrrolo[2,1-f][1,2,4]triazina-1(5,4)-pyrimidina-4(1,2)-benzenacyclononaphane

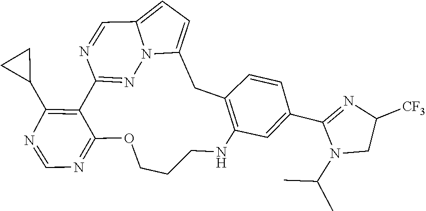 [SMILES: CC(C)N1CC(C(F)(F)F)N=C1c1ccc2c(c1)NCCCOc1ncnc(C3CC3)c1-c1ncc3ccc(n3n1)C2]

### Step 1 2-((2-(4-cyclopropyl-6-methoxypyrimidin-5-yl)pyrrolo[2,1-f][1,2,4]triazin-7-yl)methyl)-5-(1-isopropyl-4-(rifluoromethyl)-1H-imidazol-2-yl)phenol

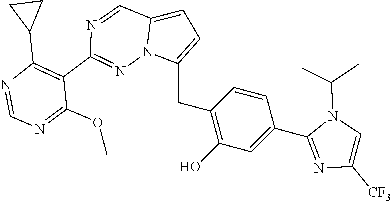 [SMILES: COc1ncnc(C2CC2)c1-c1ncc2ccc(Cc3ccc(-c4nc(C(F)(F)F)cn4C(C)C)cc3O)n2n1]

Starting with the product of step 6 in Example 21 and the product of Step 5 in Example 3, the title product was obtained as a yellow solid by following the same procedures as described in Step 7 and Step 8 of Example 21. LCMS (ESI): 550.3 [M+1]⁺.

### Step 2 2-((2-(4-cyclopropyl-6-methoxypyrimidin-5-yl)pyrrolo[2,1-f][1,2,4]triazin-7-yl) methyl)-5-(1-isopropyl-4-(trifluoromethyl)-1H-imidazol-2-yl)phenyltrifluoromethanesulfonate

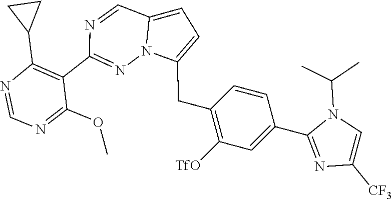 [SMILES: COc1ncnc(C2CC2)c1-c1ncc2ccc(Cc3ccc(-c4nc(C(F)(F)F)cn4C(C)C)cc3OS(=O)(=O)C(F)(F)F)n2n1]

Starting with the product of Step 1, the title product was obtained as a yellow solid by following the same procedures as described in Step 1 of Example 32. LCMS (ESI): 682.3 [M+1]⁺.

### Step 3 N-(3-((tert-butyldimethylsilyl)oxy)propyl)-2-((2-(4-cyclopropyl-6-methoxypyrimidin-5-yl) pyrrolo[2,1-f][1,2,4]triazin-7-yl)methyl)-5-(1-isopropyl-4-(trifluoromethyl)-1H-imidazol-2-yl)aniline

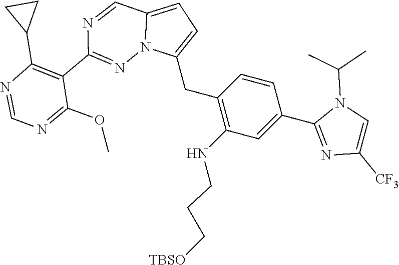 [SMILES: COc1ncnc(C2CC2)c1-c1ncc2ccc(Cc3ccc(-c4nc(C(F)(F)F)cn4C(C)C)cc3NCCCO[Si](C)(C)C(C)(C)C)n2n1]

A solution of the product of Step 2 (300 mg), 3-((tert-butyldimethylsilyl)oxy)propan-1-amine (125 mg), Pd(OAc)₂ (10 mg), 2-(dicyclohexylphosphino)-2′,4′,6′-tri-i-propyl-1,1′-biphenyl (42 mg), Cs₂CO₃ (431 mg) in toluene (5 mL) was stirred at 110° C. for 1 h under nitrogen. The mixture was then concentrated and the residue was purified by SGCC (DCM/methanol: 20/1) to obtain the product (172 mg) as yellow solid. LCMS (ESI): 721.3 [M+1]⁺.

### Step 4 (Z)-1⁶-cyclopropyl-4⁴-(1-isopropyl-4-(trifluoromethyl)-1H-imidazol-2-yl)-9-oxa-5-aza-2(2,7)-pyrrolo[2,1-f][1,2,4]triazina-1(5,4)-pyrimidina-4(1,2)-benzenacyclononaphane

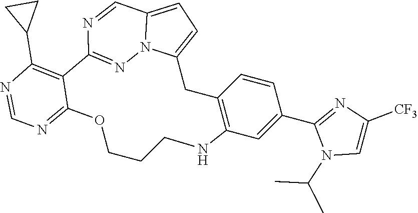 [SMILES: CC(C)n1cc(C(F)(F)F)nc1-c1ccc2c(c1)NCCCOc1ncnc(C3CC3)c1-c1ncc3ccc(n3n1)C2]

Starting with the product of Step 3, the title product was obtained as white solid by following the same procedures as described in Step 13 and Step 14 of Example 3. LCMS (ESI): 575 [M+1]⁺.

### Example 34

### (Z)-1⁶-cyclopropyl-4⁴-(1-cyclopropyl-4-(trifluoromethyl)-1H-imidazol-2-yl)-2⁵-fluoro-5,8-dioxa-2(2,7)-pyrrolo[2,1-f][1,2,4]triazina-1(5,4)-pyrimidina-4(1,2)-benzenacyclooctaphane

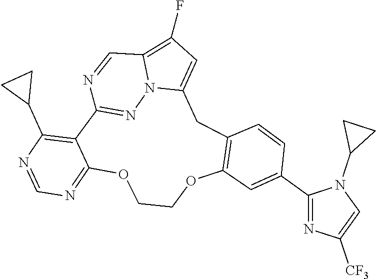 [SMILES: Fc1cc2n3nc(ncc13)-c1c(ncnc1C1CC1)OCCOc1cc(-c3nc(C(F)(F)F)cn3C3CC3)ccc1C2]

To a mixture of Example 21 (400 mg) in acetonitrile (10 mL) was added 1-chloromethyl-4-fluoro-1,4-diazoniabicyclo[2.2.2]octane bis(tetrafluoroborate) (254 mg). After stirring at r.t. for 1 h, the reaction mixture was quenched with i-Pr₂NEt (1 mL) and 50 mL water. The mixture was extracted with EtOAc (3×15 mL). The combined organic layer was dried over anhydrous Na₂SO₄ and concentrated. The residue was purified by SGCC (PE/EtOAc: from 100:0 to 2:1) to afford the title compound as yellow solid (15 mg). LCMS (ESI): 578.4 [M+1]⁺.

### Example 35

1⁶-cyclopropyl-4⁴-(1-isopropyl-4-(trifluoromethyl)-1H-imidazol-2-yl)-2⁷H-9-oxa-5-aza-2(2,7)-pyrrolo[2,3-d]pyrimidina-1(5,4)-pyrimidina-4(1,2)-benzenacyclononaphane

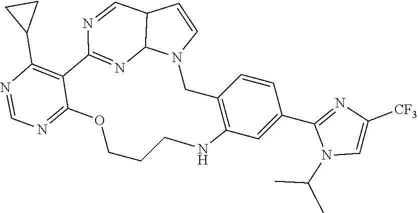 [SMILES: CC(C)n1cc(C(F)(F)F)nc1-c1ccc2c(c1)NCCCOc1ncnc(C3CC3)c1C1=NC3C(C=CN3C2)C=N1]

### Step 1 7-(2-(benzyloxy)-4-(1-isopropyl-4-(trifluoromethyl)-1H-imidazol-2-yl)benzyl)-2-chloro-7H-pyrrolo[2,3-d]pyrimidine

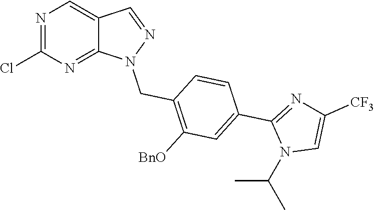 [SMILES: CC(C)n1cc(C(F)(F)F)nc1-c1ccc(Cn2ncc3cnc(Cl)nc32)c(OCc2ccccc2)c1]

Substituting 6-chloro-1H-pyrazolo[3,4-d]pyrimidine and the product of Step 2 in Example 6 with 2-chloro-7H-pyrrolo[2,3-d]pyrimidine and the product of Step 5 in Example 16, the title compound was prepared under the same conditions as described in Step 3 of Example 6. LCMS (ESI): 526 [M+1]⁺.

### Step 2 7-(2-(benzyloxy)-4-(1-isopropyl-4-(trifluoromethyl)-1H-imidazol-2-yl)benzyl)-2-(4-cyclopropyl-6-methoxypyrimidin-5-yl)-7H-pyrrolo[2,3-d]pyrimidine

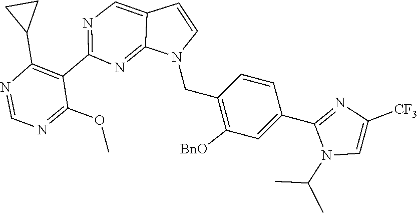 [SMILES: COc1ncnc(C2CC2)c1-c1ncc2ccn(Cc3ccc(-c4nc(C(F)(F)F)cn4C(C)C)cc3OCc3ccccc3)c2n1]

Substituting the product of Step 7 in Example 3 with the product of Step 1, the title compound was prepared under the same conditions as described in Step 8 of Example 3. LCMS (ESI): 640 [M+1]⁺.

### Step 3 2-((2-(4-cyclopropyl-6-methoxypyrimidin-5-yl)-7H-pyrrolo[2,3-d]pyrimidin-7-yl)methyl)-5-(1-isopropyl-4-(trifluoromethyl)-1H-imidazol-2-yl)phenol

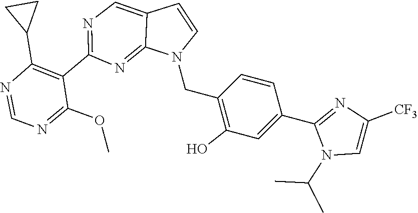 [SMILES: COc1ncnc(C2CC2)c1-c1ncc2ccn(Cc3ccc(-c4nc(C(F)(F)F)cn4C(C)C)cc3O)c2n1]

Substituting the product of Step 7 in Example 21 with the product of Step 2, the title compound was prepared under the same conditions as described in Example 21. LCMS (ESI): 550 [M+1]⁺.

### Step 4 1⁶-cyclopropyl-4⁴-(1-isopropyl-4-(trifluoromethyl)-1H-imidazol-2-yl)-2⁷H-9-oxa-5-aza-2(2,7)-pyrrolo[2,3-d]pyrimidina-1(5,4)-pyrimidina-4(1,2)-benzenacyclononaphane

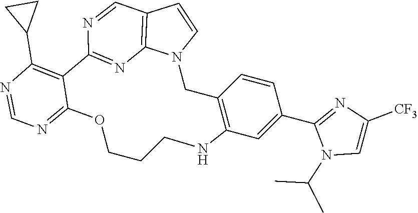 [SMILES: CC(C)n1cc(C(F)(F)F)nc1-c1ccc2c(c1)NCCCOc1ncnc(C3CC3)c1-c1ncc3ccn(c3n1)C2]

Substituting the product of Step 1 in Example 33 with the product of Step 3, the title compound was prepared under the same conditions as described in Example 33. LCMS (ESI): 575.3 [M+1]⁺.

### Example 36

### 1⁶-cyclopropyl-4⁴-(5-isopropyl-3-(trifluoromethyl)-1H-pyrazol-1-yl)-2¹H-9-oxa-5-aza-2(6,1)-pyrazolo[3,4-d]pyrimidina-1(5,4)-pyrimidina-4(1,2)-benzenacyclononaphane

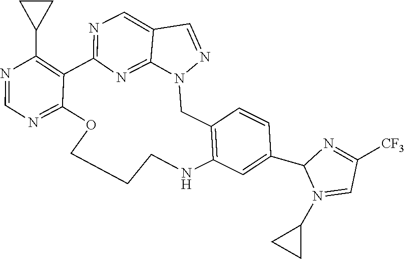

### Step 1 6-(4-cyclopropyl-6-(3,3-diethoxypropoxy)pyrimidin-5-yl)-1H-pyrazolo[3,4-d]pyrimidine

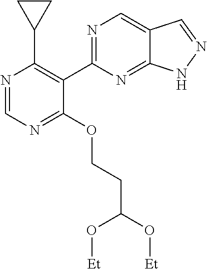 [SMILES: CCOC(CCOc1ncnc(C2CC2)c1-c1ncc2cn[nH]c2n1)OCC]

To mixture of the product of Step 9 in Example 3 (1.5 g), 4,4-diethoxybutan-1-ol (1.4 g) in dioxane (15 mL) was added NaH (1.3 g) in three portions. The reaction solution was stirred at 90° C. for 2 h, and then was added dropwise into water. The mixture was extracted with EtOAc, washed with brine, dried over MgSO₄ and concentrated under vacuum. The residue was purified by SGCC (EA/PE=0 to 20%) to afford the title compound as white solid (1.23 g). LCMS (ESI): 385 [M+1]⁺.

### Step 2 1-(4-bromo-2-nitrobenzyl)-6-(4-cyclopropyl-6-(3,3-diethoxypropoxy)pyrimidin-5-yl)-1H-pyrazolo[3,4-d]pyrimidine

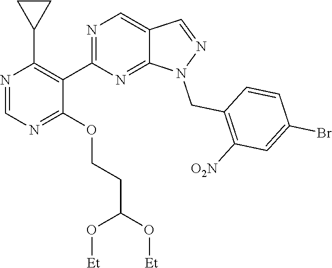 [SMILES: CCOC(CCOc1ncnc(C2CC2)c1-c1ncc2cnn(Cc3ccc(Br)cc3[N+](=O)[O-])c2n1)OCC]

To a solution of the product of step 1 (1.0 g) and (4-bromo-2-nitrophenyl)methanol (0.77 g) in THE (15 mL) were added Ph₃P (1.02 g), DIAD (0.8 g) at 0° C. under N₂ atmosphere. The reaction mixture was stirred for 4 h, concentrated and purified by SGCC (EA/PE=0 to 20%) to give the title compound (1.0 g) as yellow oil. LCMS (ESI): 598 [M+1]⁺.

### Step 3 5-bromo-2-((6-(4-cyclopropyl-6-(3,3-diethoxypropoxy)pyrimidin-5-yl)-1H-pyrazolo[3,4-d]pyrimidin-1-yl)methyl)aniline

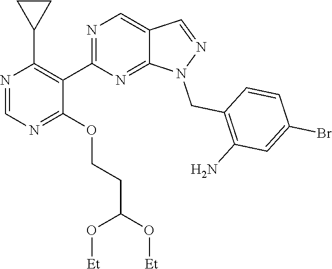 [SMILES: CCOC(CCOc1ncnc(C2CC2)c1-c1ncc2cnn(Cc3ccc(Br)cc3N)c2n1)OCC]

To a solution of the product of step 2 (1.0 g) in DMF (10 mL) were added 4,4-bipyridine (30 mg) and B₂(OH)₄ (0.7 g). The reaction mixture was stirred at 30° C. for 60 minutes and then poured into water (10 mL) and extracted with EtOAc (500 mL×2). The combined organic layer was washed with brine (100 mL), dried over Na₂SO₄ and concentrated under vacuum. The residue was purified by SGCC eluted with EtOAc/PE (0 to 30% EtOAc) to afford the title product as yellow solid (0.9 g). LCMS (ES+): 568 [M+1]⁺.

### Step 4 5-bromo-N-(3-((tert-butyldimethylsilyl)oxy)propyl)-2-((6-(4-cyclopropyl-6-(3,3-diethoxypropoxy)pyrimidin-5-yl)-1H-pyrazolo[3,4-d]pyrimidin-1-yl)methyl)aniline

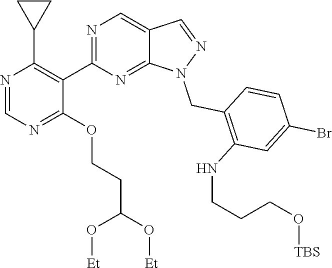

To a solution of the product of step 3 (0.9 g) and (but-3-en-1-yloxy)(tert-butyl)dimethylsilane(0.8 g) in 1,2-dichloroethane (10 mL) were added NaBH(OAc)₃ (0.8 g) at rt. After stirring at rt for 1 h, the reaction mixture was concentrated and extracted with EA (50 mL×2). The combined organic phase was combined and washed with brine, dried over Na₂SO₄, filtrated and concentrated. The residue was purified by SGCC (EA:PE=0 to 30%) to give the title compound (0.8 g) as yellow solid. LCMS (ES+): 740 [M+1]⁺.

### Step 5 3-((5-bromo-2-((6-(4-cyclopropyl-6-(3,3-diethoxypropoxy)pyrimidin-5-yl)-1H-pyrazolo[3,4-d]pyrimidin-1-yl)methyl)phenyl)amino)propan-1-ol

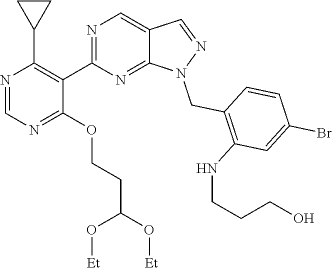 [SMILES: CCOC(CCOc1ncnc(C2CC2)c1-c1ncc2cnn(Cc3ccc(Br)cc3NCCCO)c2n1)OCC]

To a solution of the product of step 4 (1.0 g) in DMF (10 mL) was added TBAF (0.8 g). After stirring at 30° C. for 5 h, the reaction mixture was poured into water (200 mL), extracted by EtOAc (50 mL×3). The combined organic phase was washed with brine, dried over Na₂SO₄, filtrated and concentrated to give the crude title compound (0.7 g) as brown solid. LCMS (ESI): 626 [M+1]⁺.

### Step 6 44-bromo-16-cyclopropyl-21H-9-oxa-5-aza-2(6,1)-pyrazolo[3,4-d]pyrimidina-1(5,4)-pyrimidin-4(1,2)-benzenacyclononaphane

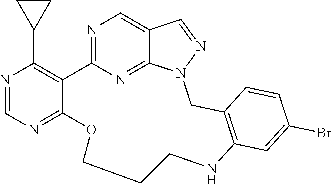 [SMILES: Brc1ccc2c(c1)NCCCOc1ncnc(C3CC3)c1-c1ncc3cnn(c3n1)C2]

To a solution of the product of step 5 (0.7 g) in 1.4-dixoane (5 mL) was added NaH (0.4 g). The reaction mixture was stirred at 80° C. for 5 h., then poured into H₂O (200 mL) and extracted with EtOAc (50 mL×3). The combined organic phase was washed with brine, dried over Na₂SO₄, filtrated and concentrated. The residue was purified by SGCC (PE:EA=0 to 20%) to give the title compound (0.29 g) as brown solid. LCMS (ESI): 478 [M+1]⁺.

### Step 7 1-cyclopropyl-4-(5-cyclopropyl-3-(trifluoromethyl)-1H-pyrazol-1-yl)-21H-9-oxa-5-aza-2(6,1)-pyrazolo[3,4-d]pyrimidina-1(5,4)-pyrimidina-4(1,2)-benzenacyclononaphane

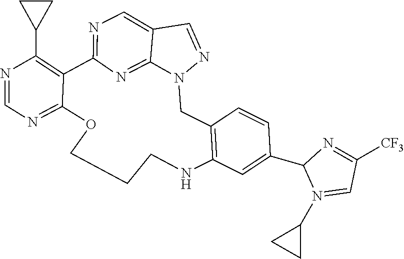

To a solution of the product of step 6 (50 mg) and 5-cyclopropyl-3-(trifluoromethyl)-1H-pyrazole (35.2 mg) in DMSO (2 mL) were added Cu₂O (2 mg), K₃PO₄ (63.6 mg) and N,N-bis(furan-2-ylmethyl)oxamide (2 mg). The reaction mixture was stirred at 130° C. for 16 h, then poured into water (100 mL) and extracted by EtOAc (150 mL×3). The combined organic phase was washed with brine, dried over Na₂SO₄, filtrated and concentrated. The residue was purified by SGSS (PE:EA=0 to 20%) to give the title compound (11 mg) as light brown solid. LCMS (ESI): 574 [M+1]⁺.

### Example 37

### 16-cyclopropyl-44-(5-isopropyl-3-(trifluoromethyl)-1H-pyrazol-1-yl)-21H-9-oxa-5-aza-2(6,1)-pyrazolo[3,4-d]pyrimidina-1(5,4)-pyrimidina-4(1,2)-benzenacyclononaphane

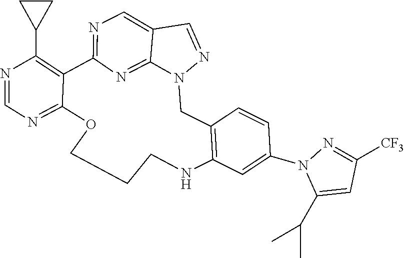 [SMILES: CC(C)c1cc(C(F)(F)F)nn1-c1ccc2c(c1)NCCCOc1ncnc(C3CC3)c1-c1ncc3cnn(c3n1)C2]

Substituting 5-cyclopropyl-3-(trifluoromethyl)-1H-pyrazole with 5-isopropyl-3-(trifluoromethyl)-1H-pyrazole, the title compound was prepared under the same conditions as described for Example 36.

LCMS (ESI): 576 [M+1]⁺.

### Example 38

### 1⁶-cyclopropyl-4⁴-(1-isopropyl-4-(trifluoromethyl)-1H-imidazol-2-yl)-2¹H-5,9-dioxa-2(6,1)-pyrazolo[3,4-d]pyrimidina-1(5,4)-pyrimidina-4(1,3)-benzenacyclononaphane

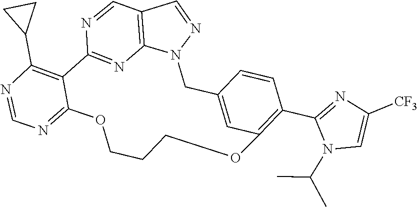 [SMILES: CC(C)n1cc(C(F)(F)F)nc1-c1ccc2cc1OCCCOc1ncnc(C3CC3)c1-c1ncc3cnn(c3n1)C2]

Substituting 3-((tert-butyldimethylsilyl)oxy)propanol with 3-((tert-butyldimethylsilyl)oxy) propanol, the title compound was prepared under the same conditions as described for Example 29. LCMS (ESI): 577 [M+1]⁺.

### Example 39

### (2¹Z,5E)-1⁶-cyclopropyl-4⁴-(1-cyclopropyl-4-(trifluoromethyl)-1H-imidazol-2-yl)-8-oxa-2(2,7)-pyrrolo[2,1-f][1,2,4]triazina-1(5,4)-pyrimidina-4(1,2)-benzenacyclooctaphan-5-ene

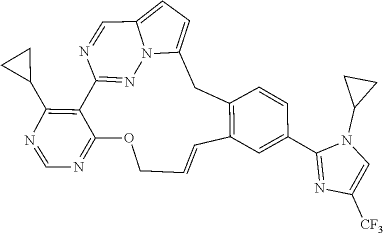 [SMILES: FC(F)(F)c1cn(C2CC2)c(-c2ccc3c(c2)/C=C/COc2ncnc(C4CC4)c2-c2ncc4ccc(n4n2)C3)n1]

Substituting the product of Step 1 in Example 30 with the product of Step 1 in Example 32, the title compound was prepared under the same conditions as described for Example 30. LCMS (ESI): 556 [M+1]⁺.

### Example 40

### 1⁶-cyclopropyl-4⁴-(1-isopropyl-4-(trifluoromethyl)-1H-imidazol-2-yl)-2H-8-oxa-5-aza-2(6,1)-pyrazolo[3,4-d]pyrimidina-1(5,4)-pyrimidina-4(1,3)-benzenacyclooctaphane

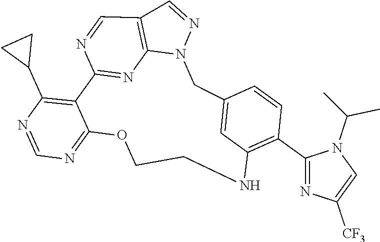 [SMILES: CC(C)n1cc(C(F)(F)F)nc1-c1ccc2cc1NCCOc1ncnc(C3CC3)c1-c1ncc3cnn(c3n1)C2]

### Step 1 5-((6-(4-cyclopropyl-6-methoxypyrimidin-5-yl)-1H-pyrazolo[3,4-d]pyrimidin-1-yl)methyl)-2-(1-isopropyl-4-trifluoromethyl)-1H-imidazol-2-yl)phenol

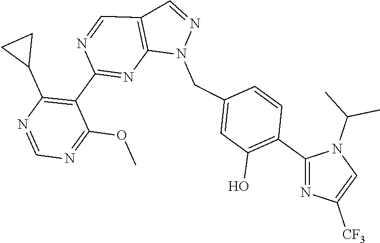 [SMILES: COc1ncnc(C2CC2)c1-c1ncc2cnn(Cc3ccc(-c4nc(C(F)(F)F)cn4C(C)C)c(O)c3)c2n1]

Starting with the product of Step 3 in Example 29 and the product of Step 9 in Example 3, the title product was obtained as brown solid by following the same procedures as described in Step 10 and Step 11 of Example 3. LCMS (ESI): 551 [M+1]⁺.

### Step 2 5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)phenyl trifluoromethanesulfonate

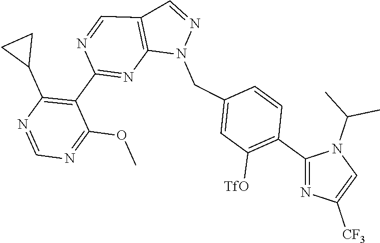 [SMILES: COc1ncnc(C2CC2)c1-c1ncc2cnn(Cc3ccc(-c4nc(C(F)(F)F)cn4C(C)C)c(OS(=O)(=O)C(F)(F)F)c3)c2n1]

To a solution of the product of Step 1 (0.6 g), triethylamine (0.3 g) in DCM (10 mL) at 0° C. were added trifluoromethanesulfonic anhydride (0.5 g). After stirring at rt for 1 h, the reaction mixture was concentrated and the residue was purified by SGCC eluted with EtOAc/PE (1/1) to obtain the product as yellow solid (0.6 g). LCMS (ESI): 683.3 [M+1]⁺.

### Step 3 N-(2-((tert-butyldimethylsilyl)oxy)ethyl)-5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)aniline

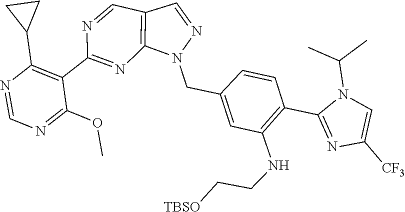 [SMILES: COc1ncnc(C2CC2)c1-c1ncc2cnn(Cc3ccc(-c4nc(C(F)(F)F)cn4C(C)C)c(NCCO[Si](C)(C)C(C)(C)C)c3)c2n1]

A solution of the product of step 2 (0.3 g), 2-((tert-butyldimethylsilyl)oxy)ethan-1-amine (0.1 g), Pd(OAc)₂ (0.02 g), 1.1′-binaphthyl-2.2′-diphemyl phosphine (0.1 g), Cs₂CO₃ (0.4 g) in 1,4-dioxane (10 mL) was stirred at 100° C. for 2 h under nitrogen. The reaction mixture was then concentrated and the residue was purified by SGCC eluted with EtOAc/PE (1/1) to obtain the product as yellow solid (0.3 g). LCMS (ESI): 708.5 [M+1]⁺.

### Step 4 2-((5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)phenyl)amino)ethan-1-ol

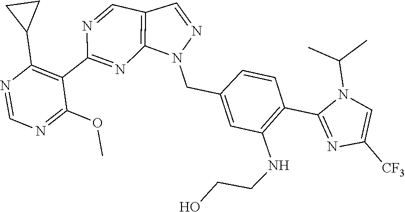 [SMILES: COc1ncnc(C2CC2)c1-c1ncc2cnn(Cc3ccc(-c4nc(C(F)(F)F)cn4C(C)C)c(NCCO)c3)c2n1]

A solution of the product of Step 3 (0.3 g) in 1,4-dioxane/3M aq. HCl (5/5 mL) was stirred at rt for 0.5 h. The reaction mixture was adjusted pH to 7 with K₂CO₃ (aq) and extracted with EA (3×30 mL). The organic layer was washed with brine (3×30 mL) and concentrated to obtain the crude product as yellow solid (0.2 g). LCMS (ESI): 594.4 [M+1]⁺.

### Step 5 1⁶-cyclopropyl-4⁴-(1-isopropyl-4-(trifluoromethyl)-1H-imidazol-2-yl)-2¹H-8-oxa-5-aza-2(6,1)-pyrazolo[3,4-d]pyrimidina-1(5,4)-pyrimidina-4(1,3)-benzenacyclooctaphane

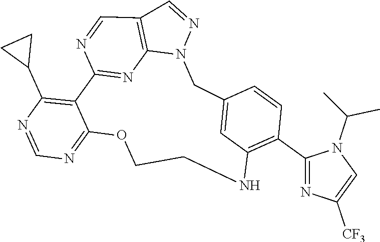 [SMILES: CC(C)n1cc(C(F)(F)F)nc1-c1ccc2cc1NCCOc1ncnc(C3CC3)c1-c1ncc3cnn(c3n1)C2]

A solution of the product of Step 4 (0.2 g), NaH (0.2 g), 1,4,7,10,13-pentaoxacyclopentadecane (0.3 g) in 1,4-dioxane (10 mL) was stirred at 90° C. for 1 h. The reaction mixture was then cooled to rt, quenched with water (10 mL) and extracted with EA (3×30 mL). The combined organic layer was washed with brine and concentrated. The residue was purified by SGCC eluted with EtOAc/PE (1/1) to obtain the product as yellow solid (0.06 g). LCMS (ESI): 562.3 [M+1]⁺. ¹H NMR (500 MHz, CDCl₃) δ 9.31 (s, 1H), 8.68 (s, 1H), 8.16 (s, 1H), 7.81 (s, 1H), 7.41 (s, 1H), 7.03 (d, 1H), 6.87 (d, 1H), 5.78 (s, 1H), 5.71 (s, 2H), 4.68 (s, 2H), 4.49-4.40 (m, 1H), 3.58 (s, 2H), 2.31-2.17 (m, 1H), 1.41 (d, 6H), 1.35-1.31 (m, 2H), 1.08-0.98 (m, 2H).

### Example 41

### (Z)-1⁶-cyclopropyl-4⁴-(1-cyclopropyl-4-(trifluoromethyl)-1H-imidazol-2-yl)-9-oxa-5-aza-2(2,7)-pyrrolo[2,1-f][1,2,4]triazina-1(5,4)-pyrimidina-4(1,2)-benzenacyclononaphane

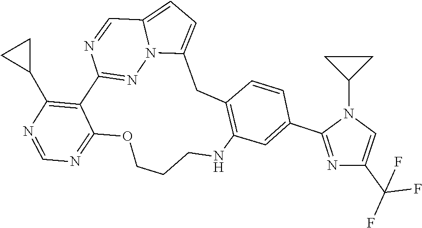 [SMILES: FC(F)(F)c1cn(C2CC2)c(-c2ccc3c(c2)NCCCOc2ncnc(C4CC4)c2-c2ncc4ccc(n4n2)C3)n1]

### Step 1 5-(1-cyclopropyl-4-(trifluoromethyl)-1H-imidazol-2-yl)-2-((2-(4-cyclopropyl-6-methoxypyrimidin-5-yl) pyrrolo[2,1-f][1,2,4]triazin-7-yl)methyl)aniline

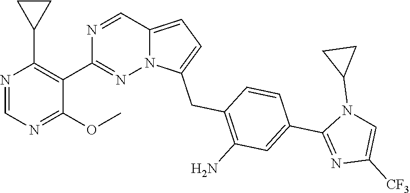 [SMILES: COc1ncnc(C2CC2)c1-c1ncc2ccc(Cc3ccc(-c4nc(C(F)(F)F)cn4C4CC4)cc3N)n2n1]

A solution of the product of Step 1 in EXAMPLE 32 (400 mg), tert-butyl carbamate (103 mg), tris(dibenzylideneacetone)dipalladium (108 mg), 2-(dicyclohexylphosphino)-2′,4′,6′-tri-i-propyl-1,1′-biphenyl (112 mg), Cs₂CO₃ (576 mg) in 1,4-dioxane (12 mL) was stirred at 90° C. for 3 h under nitrogen. The reaction mixture was then treated with 1,4-dioxane/6M HCl (12/10 mL). After stirring at r.t. for 2 h, the mixture was adjusted to pH 7 by using K₂CO₃ and diluted with EA (30 mL), washed with brine. The organic extract was concentrated, and the residue was purified by SGCC (PE/EA: 1/1) to obtain the product (149 mg) as a yellow solid. LCMS (ESI): 547.3 [M+1]⁺.

### Step 2 N-(3-((tert-butyldimethylsilyl)oxy)propyl)-5-(1-cyclopropyl-4-(trifluoromethyl)-1H-imidazol-2-yl)-2-((2-(4-cyclopropyl-6-methoxypyrimidin-5-yl)pyrrolo[2,1-f][1,2,4]triazin-7-yl)methyl)aniline

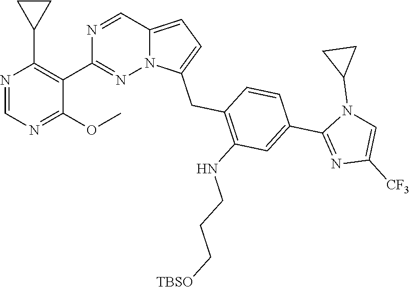 [SMILES: COc1ncnc(C2CC2)c1-c1ncc2ccc(Cc3ccc(-c4nc(C(F)(F)F)cn4C4CC4)cc3NCCCO[Si](C)(C)C(C)(C)C)n2n1]

A solution of the product of step 1 (110 mg), 3-((tert-butyldimethylsilyl)oxy)propanal (45 mg) and NaBH(OAc)₃ (128 mg) in DCM (3 mL) was stirred at r.t. for 30 min. The reaction mixture was washed with aq. K₂CO₃ and brine. The organic phase was concentrated to afford the crude product (144 mg) as a yellow solid. LCMS (ESI): 719.2 [M+1]⁺.

### Step 3 6-cyclopropyl-5-(7-(4-(1-cyclopropyl-4-(trifluoromethyl)-1H-imidazol-2-yl)-2-((3-hydroxypropyl)amino)benzyl)pyrrolo[2,1-J][1,2,4]triazin-2-yl)pyrimidin-4-ol

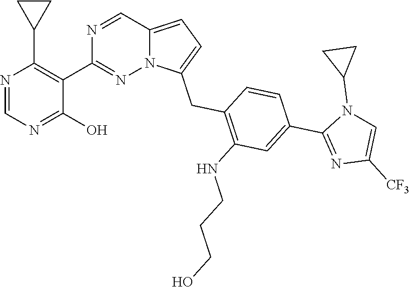 [SMILES: OCCCNc1cc(-c2nc(C(F)(F)F)cn2C2CC2)ccc1Cc1ccc2cnc(-c3c(O)ncnc3C3CC3)nn12]

A solution of the product of step 2 (144 mg) in 1,4-dioxane/6M aq. HCl (3/3 mL) was stirred at 80° C. for 1 h. The mixture was adjusted to pH 7 using K₂CO₃ (aq) and extracted with EA (30 mL), washed with brine and concentrated to obtain the product (118 mg) as a yellow solid. LCMS (ESI): 591.3 [M+1]⁺.

### Step 4 (Z)-1⁶-cyclopropyl-4⁴-(1-cyclopropyl-4-(trifluoromethyl)-1H-imidazol-2-yl)-9-oxa-5-aza-2(2,7)-pyrrolo[2,1-f][1,2,4]triazina-1(5,4)-pyrimidina-4(1,2)-benzenacyclononaphane

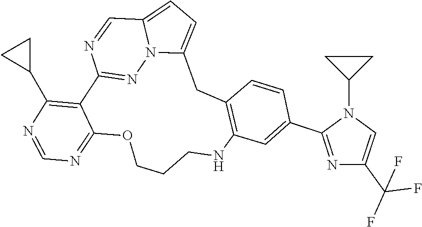 [SMILES: FC(F)(F)c1cn(C2CC2)c(-c2ccc3c(c2)NCCCOc2ncnc(C4CC4)c2-c2ncc4ccc(n4n2)C3)n1]

To a solution of the product of step 3 (118 mg), Ph₃P (210 mg) in 50 mL dry THF, DIAD (121 mg) was added. The reaction mixture was stirred at r.t. for 10 min and then concentrated. The residue was purified by SGCC (DCM/MeOH: 20/1) and the product was further purified by reverse phase column chromatography (C-18 column, ACN/H₂O: 56%) to afford the product (48 mg) as a yellow solid. LCMS (ESI): 573.3 [M+1]⁺.

¹H NMR (500 MHz, CDCl₃) δ 9.00 (s, 1H), 8.69 (s, 1H), 7.45 (d, 1H), 7.31 (s, 1H), 7.17 (d, 1H), 7.07 (s, 1H), 6.99 (d, 1H), 6.95 (d, 1H), 5.21 (t, 1H), 4.67 (brs, 2H), 4.42 (brs, 2H), 3.54-3.48 (m, 1H), 3.46 (s, 2H), 2.27-2.18 (m, 1H), 2.11-2.04 (m, 2H), 1.10-1.03 (m, 4H), 0.94-0.85 (m, 4H).

### Example 42

### (Z)-1⁶-cyclopropyl-4⁴-(1-cyclopropyl-4-(trifluoromethyl)-1H-imidazol-2-yl)-8-oxa-5-aza-2(2,7)-pyrrolo[2,1-f][1,2,4]triazina-1(5,4)-pyrimidina-4(1,2)-benzenacyclooctaphane

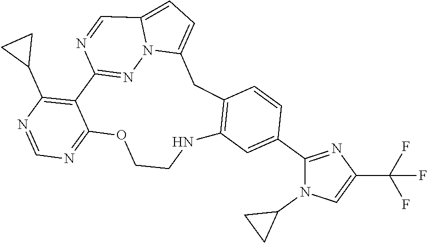 [SMILES: FC(F)(F)c1cn(C2CC2)c(-c2ccc3c(c2)NCCOc2ncnc(C4CC4)c2-c2ncc4ccc(n4n2)C3)n1]

Starting with the product of Step 1 in EXAMPLE 41 (140 mg) and 2-((tert-butyldimethylsilyl)oxy)acetaldehyde (54 mg), the title product was obtained as a yellow solid (54 mg) by following the same procedures as described for Example 41. LCMS (ESI): 559.2 [M+1]. ¹H NMR (500 MHz, CDCl₃) δ 9.01 (s, 1H), 8.73 (s, 1H), 7.43 (d, 1H), 7.31 (s, 1H), 7.19 (d, 1H), 7.05-7.01 (m, 2H), 6.96 (d, 1H), 5.88 (t, 1H), 4.67 (t, 2H), 4.40 (brs, 2H), 3.55-3.39 (m, 3H), 2.54-2.46 (m, 1H), 1.14-1.04 (m, 4H), 0.93-0.84 (m, 4H).

### Example 43

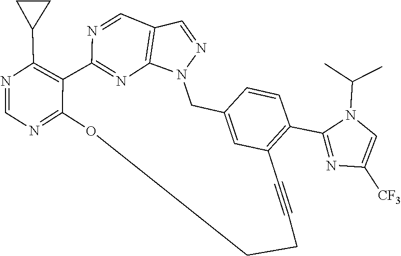 [SMILES: CC(C)n1cc(C(F)(F)F)nc1-c1ccc2cc1C#CCCOc1ncnc(C3CC3)c1-c1ncc3cnn(c3n1)C2]

### Step 11-(3-(4-((tert-butyldimethylsilyl)oxy)but-1-yn-1-yl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

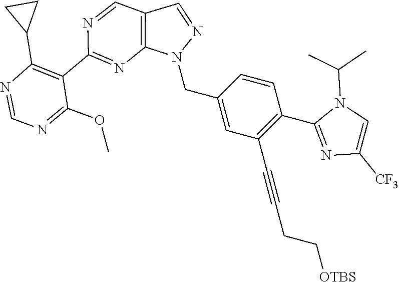 [SMILES: COc1ncnc(C2CC2)c1-c1ncc2cnn(Cc3ccc(-c4nc(C(F)(F)F)cn4C(C)C)c(C#CCCO[Si](C)(C)C(C)(C)C)c3)c2n1]

To a solution of the product of Step 2 in EXAMPLE 40 (200 mg), (Ph₃P)₂PdCl₂ (41 mg), CuI (11 mg) in THF/TEA (3/1 mL) was added (but-3-yn-1-yloxy)(tert-butyl) dimethylsilane (162 mg). The reaction mixture was stirred at 60° C. under nitrogen for 18 h and then concentrated. The residue was purified by SGCC (PE/EA: 1/1) to obtain the product (300 mg) as a white solid. LCMS (ESI): 717.3 [M+1]⁺.

### Step 2 6-cyclopropyl-5-(1-(3-(4-hydroxybut-1-yn-1-yl)-4-(1-isopropyl-4-(trifluoromethyl)-1H-imidazol-2-yl)benzyl)-1H-pyrazolo[3,4-d]pyrimidin-6-yl)pyrimidin-4-ol

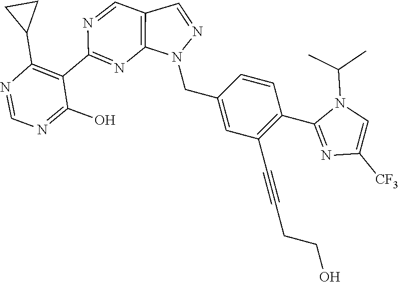 [SMILES: CC(C)n1cc(C(F)(F)F)nc1-c1ccc(Cn2ncc3cnc(-c4c(O)ncnc4C4CC4)nc32)cc1C#CCCO]

A solution of the product of step 1 (300 mg) in 1,4-dioxane/1M HCl (3/3 mL) was stirred at 80° C. for 2 h. The mixture was adjusted pH to 7 with K₂CO₃ and extracted with EA (30 mL), washed with brine and concentrated to obtain the product (156 mg) as white solid. LCMS (ESI): 589.3 [M+1]⁺.

### Step 3

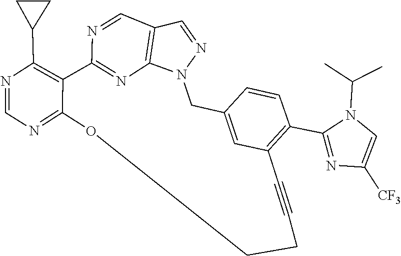 [SMILES: CC(C)n1cc(C(F)(F)F)nc1-c1ccc2cc1C#CCCOc1ncnc(C3CC3)c1-c1ncc3cnn(c3n1)C2]

To a solution of the product of step 2 (156 mg), Ph₃P (278 mg) in 50 mL dry THF, DIAD (161 mg) was added and stirred at r.t. for 10 min. The reaction mixture was then concentrated and purified by SGCC (DCM/MeOH: 20/1) and then further purified by reverse phase column chromatography (C-18 column, ACN/H₂O: 46%) to afford the product (14 mg) as white solid. LCMS (ESI): 571.4 [M+1]⁺.

### Example 44

### (E)-1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphan-5-ene

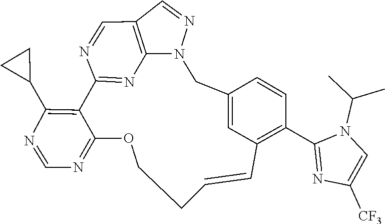 [SMILES: CC(C)n1cc(C(F)(F)F)nc1-c1ccc2cc1/C=C/CCOc1ncnc(C3CC3)c1-c1ncc3cnn(c3n1)C2]

### Step 1 (E)-1-(3-(4-((tert-butyldimethylsilyl)oxy)but-1-en-1-yl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

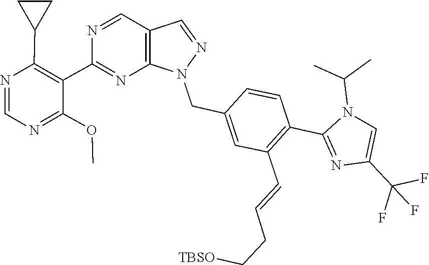 [SMILES: COc1ncnc(C2CC2)c1-c1ncc2cnn(Cc3ccc(-c4nc(C(F)(F)F)cn4C(C)C)c(/C=C/CCO[Si](C)(C)C(C)(C)C)c3)c2n1]

A solution of the product of Step 2 in EXAMPLE 40 (1100 mg), (E)-tert-butyldimethyl((4-(4,4,5,5-tetramethyl-1,3,2-dioxaborolan-2-yl)but-3-en-1-yl)oxy)silane (1006 mg), Pd₂(dba)₃ (148 mg), 2-(dicyclohexylphosphino)-2′,4′,6′-tri-i-propyl-1,1′-biphenyl (154 mg), CsF (735 mg) in 1,4-dioxane/H₂O (15/1.5 mL) was stirred at 100° C. for 1 h under nitrogen. The reaction mixture was then concentrated and purified by SGCC (PE/EA: 1/1) to obtain the product (1180 mg) as white solid. LCMS (ESI): 719.5 [M+1]⁺.

### Step 2 (E)-1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphan-5-ene

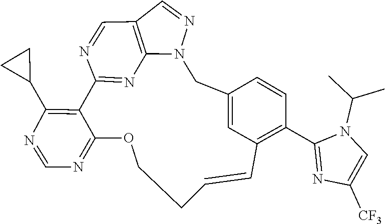 [SMILES: CC(C)n1cc(C(F)(F)F)nc1-c1ccc2cc1/C=C/CCOc1ncnc(C3CC3)c1-c1ncc3cnn(c3n1)C2]

Starting with the product of step 1 (400 mg), the title product was obtained as a white solid (32 mg) by following the same procedures as described for Step 2 and Step 3 in EXAMPLE 43. LCMS (ESI): 573.3 [M+1]⁺.

### Example 45

### 1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphane

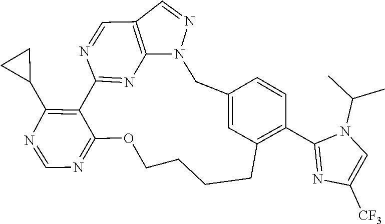 [SMILES: CC(C)n1cc(C(F)(F)F)nc1-c1ccc2cc1CCCCOc1ncnc(C3CC3)c1-c1ncc3cnn(c3n1)C2]

### Step 1 1-(3-(4-((tert-butyldimethylsilyl)oxy)butyl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

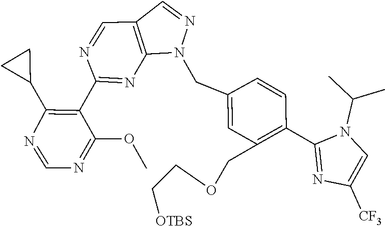 [SMILES: COc1ncnc(C2CC2)c1-c1ncc2cnn(Cc3ccc(-c4nc(C(F)(F)F)cn4C(C)C)c(COCCO[Si](C)(C)C(C)(C)C)c3)c2n1]

A solution of the product of Step 1 in EXAMPLE 44 (800 mg) and Pd/C (10% w.t) in MeOH (8 mL) was stirred under a balloon pressure of hydrogen at rt for 18 h. The mixture was then filtered and concentrated. The residue (804 mg) was dissolved in 10 mL of DCM and treated with 2,3-dichloro-5,6-dicyano-p-benzoquinone (472 mg). The mixture was stirred at rt for 5 min, and then washed with aq. NaHCO₃ (3×100 mL) and brine. The organic layer was concentrated to obtain the product (500 mg) as white solid. LCMS (ESI): 721.3 [M+1]⁺.

### Step 2 1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphane

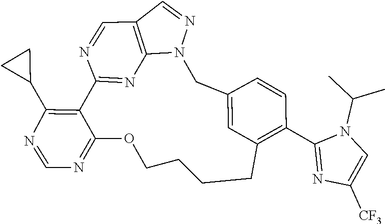 [SMILES: CC(C)n1cc(C(F)(F)F)nc1-c1ccc2cc1CCCCOc1ncnc(C3CC3)c1-c1ncc3cnn(c3n1)C2]

Starting with the product of step 1 (500 mg), the title product was obtained as a white solid (108 mg) by following the same procedures as described for Step 2 and Step 3 in EXAMPLE 43. LCMS (ESI): 575.5 [M+1].

### Example 46

### (Z)-1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphan-5-ene

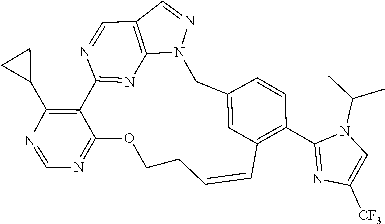 [SMILES: CC(C)n1cc(C(F)(F)F)nc1-c1ccc2cc1/C=C\CCOc1ncnc(C3CC3)c1-c1ncc3cnn(c3n1)C2]

### Step 1 (Z)-1-(3-(4-((tert-butyldimethylsilyl)oxy)but-1-en-1-yl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

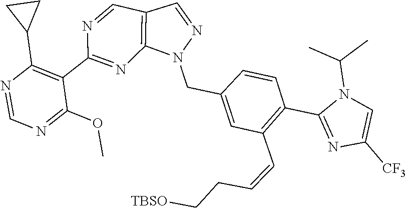 [SMILES: COc1ncnc(C2CC2)c1-c1ncc2cnn(Cc3ccc(-c4nc(C(F)(F)F)cn4C(C)C)c(/C=C\CCO[Si](C)(C)C(C)(C)C)c3)c2n1]

A solution of the product of Step 1 in EXAMPLE 43 (480 mg), Pd/BaSO₄ (5%) (2 g) in MeOH/quinoline (5 mL/2 mL) was stirred at rt for 18 h under H₂ balloon. The mixture was filtered and concentrated. The residue was purified by SGCC (PE/EA: 4/6) to afford the product (300 mg) as white solid. LCMS (ESI): 719.3 [M+1]⁺.

### Step 2 (Z)-1⁶-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,3)-benzenacyclononaphan-5-ene

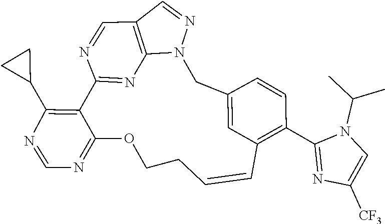 [SMILES: CC(C)n1cc(C(F)(F)F)nc1-c1ccc2cc1/C=C\CCOc1ncnc(C3CC3)c1-c1ncc3cnn(c3n1)C2]

Starting with the product of step 1 (300 mg), the title product was obtained as a white solid (73 mg) by following the same procedures as described for Step 2 and Step 3 in EXAMPLE 43. LCMS (ESI): 573.5 [M+1]⁺.

¹H NMR (400 MHz, CDCl₃) δ 9.34 (s, 1H), 8.71 (s, 1H), 8.17 (s, 1H), 7.78 (s, 1H), 7.54 (dd, 1H), 7.41-7.36 (m, 2H), 6.10 (d, 1H), 5.81 (s, 2H), 5.66-5.57 (m, 1H), 4.61 (t, 2H), 4.06-3.98 (m, 1H), 2.76-2.65 (m, 2H), 2.18-2.09 (m, 1H), 1.33 (d, 6H), 1.04-0.98 (m, 2H), 0.94-0.88 (m, 2H).

### Example 47

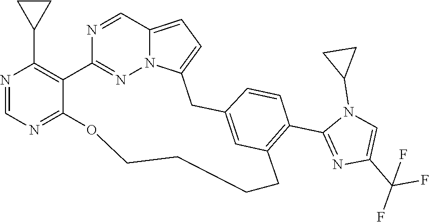 [SMILES: FC(F)(F)c1cn(C2CC2)c(-c2ccc3cc2CCCCOc2ncnc(C4CC4)c2-c2ncc4ccc(n4n2)C3)n1]

Starting with (E)-tert-butyldimethyl((4-(4,4,5,5-tetramethyl-1,3,2-dioxaborolan-2-yl)but-3-en-1-yl)oxy)silane(80 mg), the title product was obtained as a white solid by following the same procedures as described in EXAMPLE 32. LCMS (ESI): 572.1 [M+1]⁺.

### Example 48

### 1⁶-cyclopropyl-4⁴-(1-isopropyl-4-(trifluoromethyl)-1H-imidazol-2-yl)-2¹H-5,10-dioxa-2(6,1)-pyrazolo[3,4-d]pyrimidina-1(5,4)-pyrimidina-4(1,3)-benzenacyclodecaphane

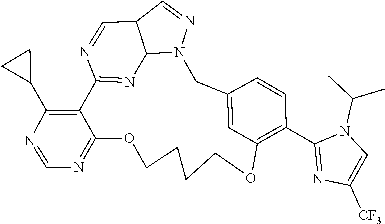 [SMILES: CC(C)n1cc(C(F)(F)F)nc1-c1ccc2cc1OCCCCOc1ncnc(C3CC3)c1C1=NC3C(C=N1)C=NN3C2]

### Step 1 5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)phenol

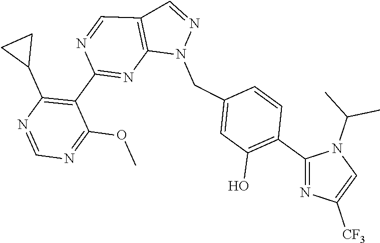 [SMILES: COc1ncnc(C2CC2)c1-c1ncc2cnn(Cc3ccc(-c4nc(C(F)(F)F)cn4C(C)C)c(O)c3)c2n1]

Starting with the product of Step 3 in EXAMPLE 29 and the product of Step 9 in EXAMPLE 3, the title product was obtained as a brown solid by following the same procedures as described in Step 10 and Step 11 of EXAMPLE 3. LCMS (ESI): 551 [M+1]⁺.

### Step 2 1-(3-(4-((tert-butyldimethylsilyl)oxy)butoxy)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

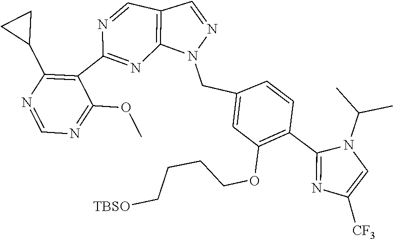 [SMILES: COc1ncnc(C2CC2)c1-c1ncc2cnn(Cc3ccc(-c4nc(C(F)(F)F)cn4C(C)C)c(OCCCCO[Si](C)(C)C(C)(C)C)c3)c2n1]

Starting with the product of Step 1 and (4-bromobutoxy)(tert-butyl)dimethylsilane, the title product was obtained as white solid by following the same procedures as described in Step 4 of EXAMPLE 29. LCMS (ESI): 737.6 [M+1]⁺.

### Step 3 1⁶-cyclopropyl-4⁴-(1-isopropyl-4-(trifluoromethyl)-1H-imidazol-2-yl)-2¹H-5,10-dioxa-2(6,1)-pyrazolo[3,4-d]pyrimidina-1(5,4)-pyrimidina-4(1,3)-benzenacyclodecaphane

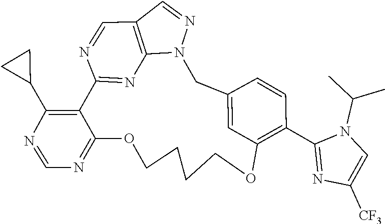 [SMILES: CC(C)n1cc(C(F)(F)F)nc1-c1ccc2cc1OCCCCOc1ncnc(C3CC3)c1-c1ncc3cnn(c3n1)C2]

Starting with the product of Step 2, the title product was obtained as white solid by following the same procedures as described in Step 5 and Step 6 of EXAMPLE 29. LCMS (ESI): 591.7 [M+1]⁺.

### Example 49

### 1⁶-cyclopropyl-4⁴-(1-isopropyl-4-(trifluoromethyl)-1H-imidazol-2-yl)-2⁷,2⁸-dihydro-5,9-dioxa-2(2,8)-pyrido[2,3-d]pyrimidina-1(5,4)-pyrimidina-4(1,3)-benzenacyclononaphan-27-one

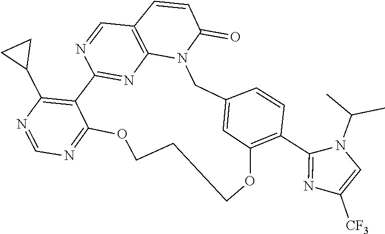 [SMILES: CC(C)n1cc(C(F)(F)F)nc1-c1ccc2cc1OCCCOc1ncnc(C3CC3)c1-c1ncc3ccc(=O)n(c3n1)C2]

### Step 1 2-(4-(chloromethyl)-2-((4-methoxybenzyl)oxy)phenyl)-1-isopropyl-4-(trifluoromethyl)-1H-imidazole

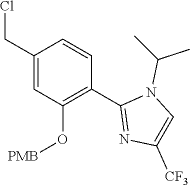

To a mixture of (4-(1-isopropyl-4-(trifluoromethyl)-1H-imidazol-2-yl)-3-((4-methoxybenzyl)oxy)phenyl)methanol (4.0 g), n-Bu₃P (3.85 g) in DCM (50 mL) was added dropwise CCl₄ (2.93 g) at rt. The reaction mixture was stirred at rt for 1 h and then quenched by aq. NaHCO₃ solution. The organic layer was washed with water (20 mL), brine (15 mL), dried over anhydrous sodium sulphate and concentrated. The residue was purified by SGCC eluted with EtOAc/PE (0 to 100% EtOAc) to afford the title product (4.0 g) as a white solid. LCMS (ESI): 439.4 [M+1]⁺.

### Step 2 2-(4-cyclopropyl-6-methoxypyrimidin-5-yl)-8-(4-(1-isopropyl-4-(trifluoromethyl)-1H-imidazol-2-yl)-3-((4-methoxybenzyl)oxy)benzyl)pyrido[2,3-d]pyrimidin-7(8H)-one

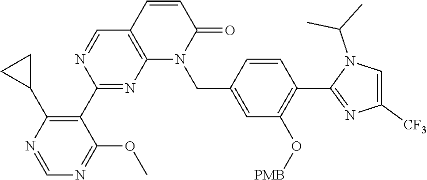

A mixture of 2-(4-cyclopropyl-6-methoxypyrimidin-5-yl)pyrido[2,3-d]pyrimidin-7(8H)-one (300 mg), the product of step 1 (490 mg) and Cs₂CO₃ (662 mg) in DMF (5.0 mL) was stirred at 50° C. for 4 h. The reaction solution was poured into water (30 mL) and extracted with EtOAc (30 mL×2). The combined organic layer was washed with brine (40 mL), dried over anhydrous Na₂SO₄ and concentrated under vacuum. The residue was purified by SGCC eluted with EtOAc/PE (o to 30% EtOAc) to afford the title product (500 mg) as a white solid. LCMS (ESI): 698.6 [M+1]⁺.

### Step 3 2-(4-cyclopropyl-6-methoxypyrimidin-5-yl)-8-(3-hydroxy-4-(1-isopropyl-4-(trifluoromethyl)-1H-imidazol-2-yl)benzyl)pyrido[2,3-d]pyrimidin-7(8H)-one

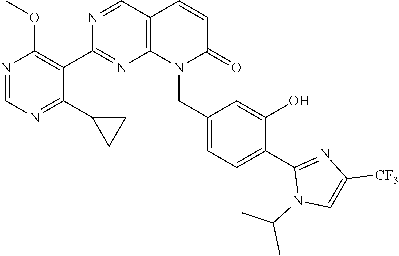 [SMILES: COc1ncnc(C2CC2)c1-c1ncc2ccc(=O)n(Cc3ccc(-c4nc(C(F)(F)F)cn4C(C)C)c(O)c3)c2n1]

A solution of the product of step 2 (500 mg) in TFA (6.0 mL) was stirred at rt for 1 h. The reaction solution was concentrated, and the residue was diluted with EtOAc (50 mL) and washed with saturated NaHCO₃ (30 mL), brine (30 mL), dried over anhydrous Na₂SO₄ and concentrated under vacuum. The residue was purified by SGCC eluted with EtOAc/PE (0 to 100% EtOAc) to afford the title product (300 mg) as a white solid. LCMS (ESI): 578.2 [M+1]⁺.

### Step 4 1⁶-cyclopropyl-4⁴-(1-isopropyl-4-(trifluoromethyl)-1H-imidazol-2-yl)-2⁷,2⁸-dihydro-5,9-dioxa-2(2,8)-pyrido[2,3-d]pyrimidina-1(5,4)-pyrimidina-4(1,3)-benzenacyclononaphan-27-one

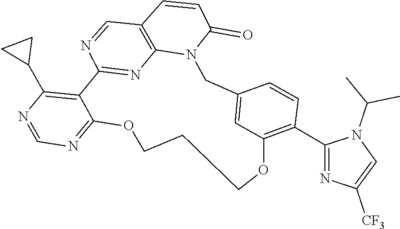 [SMILES: CC(C)n1cc(C(F)(F)F)nc1-c1ccc2cc1OCCCOc1ncnc(C3CC3)c1-c1ncc3ccc(=O)n(c3n1)C2]

Starting with (3-bromopropoxy)(tert-butyl)dimethylsilane and the product of step 3, the title product was obtained as a white solid by following the same procedures as described in Step 3, Step 4 and Step 5 of EXAMPLE 16. LCMS (ESI): 604.6 [M+1]⁺. ¹H NMR (500 MHz, DMSO-d₆) δ 9.32 (s, 1H), 8.72 (s, 1H), 8.15-8.09 (m, 2H), 7.47 (s, 1H), 7.35-7.27 (m, 2H), 6.85 (d, 1H), 5.89 (d, 1H), 5.18 (d, 1H), 4.98-4.83 (m, 1H), 4.22-4.06 (m, 3H), 4.06-3.95 (m, 1H), 2.10-1.99 (m, 1H), 1.94-1.83 (m, 1H), 1.81-1.74 (m, 1H), 1.30 (d, 6H), 1.20-1.15 (m, 1H), 1.09-0.96 (m, 2H), 0.93-0.84 (m, 1H).

### Example 50

### 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,3)-benzenacyclonona-phane

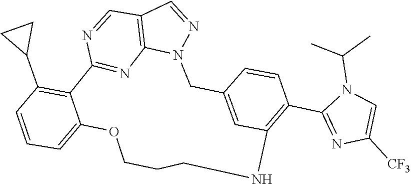 [SMILES: CC(C)n1cc(C(F)(F)F)nc1-c1ccc2cc1NCCCOc1cccc(C3CC3)c1-c1ncc3cnn(c3n1)C2]

### Step 1 N-(3-((tert-butyldimethylsilyl)oxy)propyl)-5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)aniline

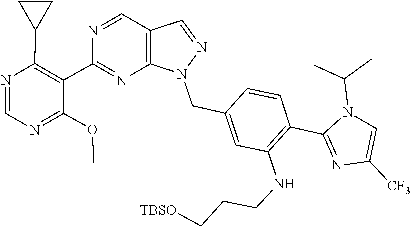 [SMILES: COc1ncnc(C2CC2)c1-c1ncc2cnn(Cc3ccc(-c4nc(C(F)(F)F)cn4C(C)C)c(NCCCO[Si](C)(C)C(C)(C)C)c3)c2n1]

A solution of the product of Step 2 in EXAMPLE 40 (0.27 g), 3-((tert-butyldimethylsilyl)oxy) propan-1-amine (0.6 g), Pd(OAc)₂ (0.02 g), 1.1′-binaphthyl-2.2′-diphemyl phosphine (0.1 g), Cs₂CO₃ (0.4 g) in 1,4-dioxane (10 mL) was stirred at 100° C. for 2 h under nitrogen. The reaction mixture was then concentrated, and the residue was purified by SGCC eluted with EtOAc/PE (1/1) to obtain the product as a yellow solid (0.23 g). LCMS (ESI): 723.2[M+1]⁺.

### Step 2 2-((5-((6-(4-cyclopropyl-6-methoxypyrimidin-5-yl)-1H-pyrazolo[3,4-d]pyrimidin-1-yl)methyl)-2-(1-isopropyl-4-(trifluoromethyl)-1H-imidazol-2-yl)phenyl)amino)ethan-1-ol

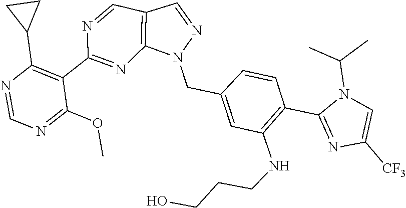 [SMILES: COc1ncnc(C2CC2)c1-c1ncc2cnn(Cc3ccc(-c4nc(C(F)(F)F)cn4C(C)C)c(NCCCO)c3)c2n1]

A solution of the product of Step 1 (0.23 g) in 1,4-dioxane/3M aq. HCl (5/5 mL) was stirred at rt for 0.5 h. The reaction mixture was adjusted to pH 7 with K₂CO₃ (aq.) and extracted with EA (3×30 mL). The organic layer was washed with brine (3×30 mL) and concentrated to obtain the crude product as a white solid (0.18 g). LCMS (ESI): 608.6 [M+1]⁺.

### Step 3 1⁶-cyclopropyl-4⁴-(1-isopropyl-4-(trifluoromethyl)-1H-imidazol-2-yl)-2¹H-9-oxa-5-aza-2(6,1)-pyrazolo[3,4-d]pyrimidina-1(5,4)-pyrimidina-4(1,3)-benzenacy-clononaphane

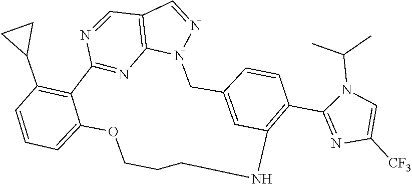 [SMILES: CC(C)n1cc(C(F)(F)F)nc1-c1ccc2cc1NCCCOc1cccc(C3CC3)c1-c1ncc3cnn(c3n1)C2]

A solution of the product of Step 2 (0.17 g), NaH (0.03 g), 1,4,7,10,13-pentaoxacyclopentadecane (0.15 g) in 1,4-dioxane (10 mL) was stirred at 90° C. for 5 h. The reaction mixture was then cooled to rt, quenched with acetic acid (2 mL), adjusted to pH 7 with K₂CO₃ (aq), and extracted with EA (3×30 mL). The combined organic layer was washed with brine and concentrated. The residue was purified by SGCC eluted with EtOAc/petroleum (1/1) to obtain the product as a yellow solid (0.08 g). LCMS (ESI): 576.6 [M+1]⁺.

### Example 51

### (2¹Z,5E)-1⁶-cyclopropyl-4⁴-(1-cyclopropyl-4-(trifluoromethyl)-1H-imidazol-2-yl)-8-oxa-2(2,7)-pyrrolo[2,1-f][1,2,4]triazina-1(5,4)-pyrimidina-4(1,2)-benzenacyclooctaphan-5-ene

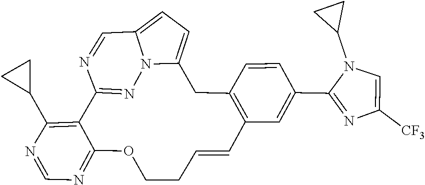 [SMILES: FC(F)(F)c1cn(C2CC2)c(-c2ccc3c(c2)/C=C/CCOc2ncnc(C4CC4)c2-c2ncc4ccc(n4n2)C3)n1]

Starting with the product of Step 1 in EXAMPLE 32, the title product was obtained as a white solid by following the same procedures as described in Step 14 of EXAMPLE 3. LCMS (ESI): 570.3 [M+1]⁺.

### Example 52

### (Z)-1⁶-cyclopropyl-4⁴-(1-cyclopropyl-4-(trifluoromethyl)-1H-imidazol-2-yl)-5,9-dioxa-2(2,7)-pyrrolo[2,1-f][1,2,4]triazina-1(5,4)-pyrimidina-4(1,3)-benzenacyclononaphane

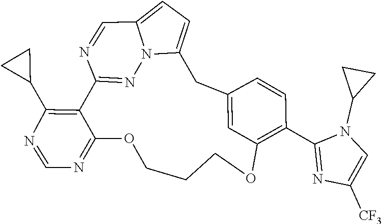 [SMILES: FC(F)(F)c1cn(C2CC2)c(-c2ccc3cc2OCCCOc2ncnc(C4CC4)c2-c2ncc4ccc(n4n2)C3)n1]

Starting with (3-bromopropoxy)(tert-butyl)dimethylsilane, the title product was obtained as a white solid by following the same procedures as described in EXAMPLE 21. LCMS (ESI): 574.7 [M+1]⁺.

### Example 53

### (Z)-25-chloro-16-cyclopropyl-44-(1-cyclopropyl-4-(trifluoromethyl)-1H-imidazol-2-yl)-5,8-dioxa-2(2,7)-pyrrolo[2,1-f][1,2,4]triazina-1(5,4)-pyrimidina-4(1,2)-benzenacyclooctaphane

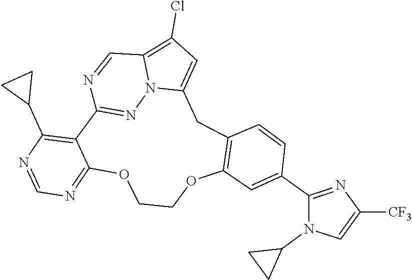 [SMILES: FC(F)(F)c1cn(C2CC2)c(-c2ccc3c(c2)OCCOc2ncnc(C4CC4)c2-c2ncc4c(Cl)cc(n4n2)C3)n1]

To a solution of EXAMPLE 21 (50 mg) in DMF (2 mL) was added NCS (13 mg). The reaction mixture was stirred at 50° C. for 1 h. The reaction mixture was quenched by 5 mL aq. saturated solution of Na₂S₂O₃ and extracted with EA (3×20 mL). The combined organic layer was washed with brine, dried over anhydrous Na₂SO₄, filtered and concentrated, The residue was purified by SGCC (PE:EA=10:0-7:3) to afford the title product (22 mg). LCMS (ESI): 594.4/596.4 [M+1]⁺.

### Example 54

### (E)-16-cyclopropyl-44-(1-isopropyl-4-(trifluoromethyl)-1H-imidazol-2-yl)-21H-9-oxa-2(6,1)-pyrazolo[3,4-d]pyrimidina-1(5,4)-pyrimidina-4(1,2)-benzenacyclononaphan-5-ene4(1,2 benzenacyclononaphane

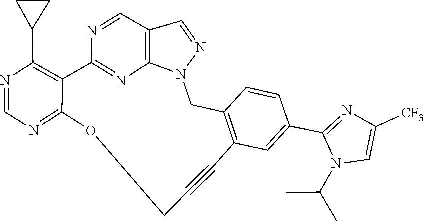 [SMILES: CC(C)n1cc(C(F)(F)F)nc1-c1ccc2c(c1)C#CCOc1ncnc(C3CC3)c1-c1ncc3cnn(c3n1)C2]

### Step 1 6-(4-cyclopropyl-6-methoxypyrimidin-5-yl)-1-(4-(1-isopropyl-4-(trifluoromethyl)-1H-imidazol-2-yl)-2-((4-methoxybenzyl)oxy)benzyl)-1H-pyrazolo[3,4-d]pyrimidine

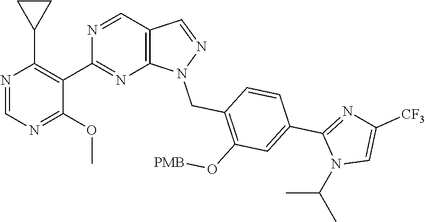

To a solution of 6-(4-cyclopropyl-6-methoxypyrimidin-5-yl)-1H-pyrazolo[3,4-d]pyrimidine (1.0 g) and (4-(1-isopropyl-4-(trifluoromethyl)-1H-imidazol-2-yl)-2-((4-methoxybenzyl)oxy)phenyl) methanol (0.77 g) in THE (10 mL) was added Ph₃P (1.1 g), DIAD (0.8 g) over a period of 30 min at 0° C. under N₂ atmosphere. The mixture was stirred at rt for 4 h and then concentrated. The residue was purified by SGCC (EA:PE=0 to 15%) to give the title compound (1.6 g) as a yellow oil. LCMS (ES+): 671 [M+1]⁺.

### Step 2 2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenol

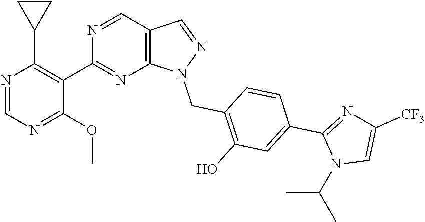 [SMILES: COc1ncnc(C2CC2)c1-c1ncc2cnn(Cc3ccc(-c4nc(C(F)(F)F)cn4C(C)C)cc3O)c2n1]

A solution of the product of step 1 (1.5 g) in TFA (10 mL) was stirred for 2 h at 100° C. The reaction mixture was concentrated, and the residue was purified by SGCC (EA:PE=0 to 15%) to give the title compound (1.1 g) as a yellow oil. LCMS (ES+): 551.21 [M+1]⁺.

### Step 3 2-((6-(4-cyclopropyl-6-methoxypyrimidin-5-yl)-1H-pyrazolo[3,4-d]pyrimidin-1-yl)methyl)-5-(1-isopropyl-4-(trifluoromethyl)-1H-imidazol-2-yl)phenyl trifluoromethanesulfonate

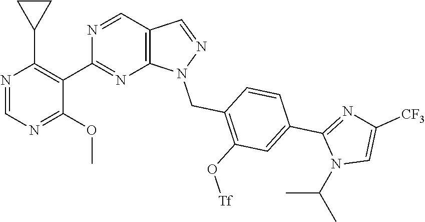

To a solution of the product of step 2 (1.0 g) in DCM (10 mL) were added trifluoromethanesulfonic anhydride (320 mg) and pyridine (0.4 g). After stirring at 0° C. for 2 h, the reaction solution was poured into water (10 mL) and extracted with DCM (5 mL×2). The combine organic layer was washed with brine (10 mL), dried over Na₂SO₄ and concentrated under vacuum. The residue was purified by SGCC eluted with EtOAc/PE (0 to 30% EtOAc) to afford the title product as a yellow solid (0.9 g). LCMS (ES+): 683.15 [M+1]⁺.

### Step 4 1-(2-(3-((tert-butyldimethylsilyl)oxy)prop-1-yn-1-yl)-4-(1-isopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

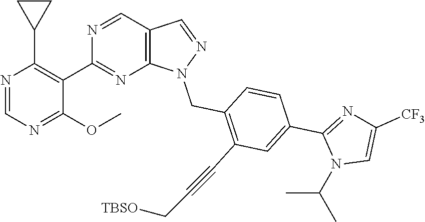 [SMILES: COc1ncnc(C2CC2)c1-c1ncc2cnn(Cc3ccc(-c4nc(C(F)(F)F)cn4C(C)C)cc3C#CCO[Si](C)(C)C(C)(C)C)c2n1]

To a solution of the product of Step 3 (0.7 g) and tert-butyldimethyl(prop-2-yn-1-yloxy)silane (0.4 g) in 1,4-dioxane (15 mL) were added Pd(dppf)Cl₂ (0.05 g) and K₂CO₃ (0.8 g) at rt. After stirring at 100° C. for 1 hours under N₂ atmosphere. The mixture was concentrated, then poured into water and extracted by EA (15 mL×3). The extracts were combined and washed with aq. NaCl, dried over Na₂SO₄, filtrated and concentrated. The residue was purified by SGCC (EA:PE=0 to 30%) to give the title compound (0.46 g) as yellow solid. LCMS (ES+): 703 [M+1]⁺.

### Step 5 6-cyclopropyl-5-(1-(2-(3-hydroxyprop-1-yn-1-yl)-4-(1-isopropyl-4-(trifluoromethyl)-1H-imidazol-2-yl)benzyl)-1H-pyrazolo[3,4-d]pyrimidin-6-yl)pyrimidin-4-ol

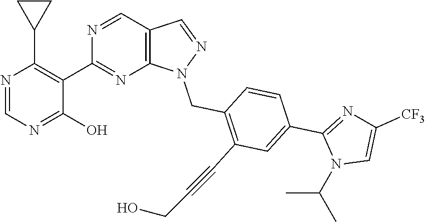 [SMILES: CC(C)n1cc(C(F)(F)F)nc1-c1ccc(Cn2ncc3cnc(-c4c(O)ncnc4C4CC4)nc32)c(C#CCO)c1]

A solution of the product of step 4 (0.2 g) in MeOH (10 mL), H₂O (10 mL) and 4N aq. HCl (5 mL) was stirred at 100° C. for 24 hours under N₂ atmosphere. The mixture was then concentrated, and adjusted pH to 8 with Na₂CO₃ solution, and extracted with EA (5 mL×2). The extracts were combined and washed with brine, dried over Na₂SO₄, filtrated and concentrated. The residue was purified by SGCC (EA:PE=0 to 30%) to give the title compound (0.14 g, 76.3%) as yellow solid. LCMS (ES+): 575 [M+1].

### Step 6 (E)-16-cyclopropyl-4⁴-(1-isopropyl-4-(trifluoromethyl)-1H-imidazol-2-yl)-2¹H-9-oxa-2(6,1)-pyrazolo[3,4-d]pyrimidina-1(5,4)-pyrimidina-4(1,2)-benzenacyclononaphan-5-ene

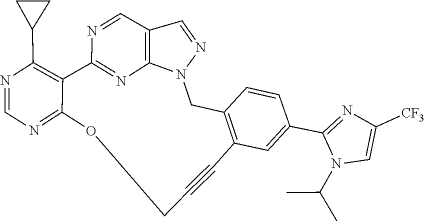 [SMILES: CC(C)n1cc(C(F)(F)F)nc1-c1ccc2c(c1)C#CCOc1ncnc(C3CC3)c1-c1ncc3cnn(c3n1)C2]

To a solution of the product of step 5 (0.05 g) in THE (5 mL) were added Ph₃P (0.02 g) and DIAD (0.08 g) over a period of 2 h at 0° C. under N₂ atmosphere. The reaction mixture was then concentrated, and the residue was purified by SGCC (EA:PE=0 to 55%) to give the title compound (0.03 g, 76.1%) as yellow oil. LCMS (ES+): 557 [M−1]⁻.

### Example 55

### (E)-16-cyclopropyl-44-(1-isopropyl-4-(trifluoromethyl)-1H-imidazol-2-yl)-21H-9-oxa-2(6,1)-pyrazolo[3,4-d]pyrimidina-1(5,4)-pyrimidina-4(1,2)-benzenacyclononaphan-5-ene4(1,2)-benzenacyclononaphane

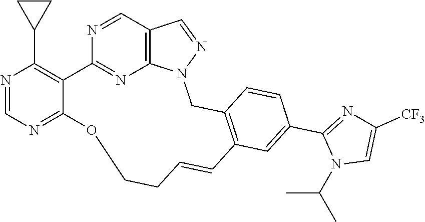 [SMILES: CC(C)n1cc(C(F)(F)F)nc1-c1ccc2c(c1)/C=C/CCOc1ncnc(C3CC3)c1-c1ncc3cnn(c3n1)C2]

The title compound was synthesized using the same procedures as described in EXAMPLE 54. LCMS (ESI): 573.7 [M+1]⁺.

### Example 56

### 1⁶-cyclopropyl-4⁴-(5-methyl-3-(trifluoromethyl)-1H-pyrazol-1-yl)-2¹H-5,9-dioxa-2(6,1)-pyrazolo[3,4-d]pyrimidina-1(5,4)-pyrimidina-4(1,3)-benzenacyclononaphane

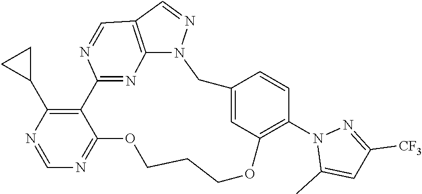 [SMILES: Cc1cc(C(F)(F)F)nn1-c1ccc2cc1OCCCOc1ncnc(C3CC3)c1-c1ncc3cnn(c3n1)C2]

### Step 1 4-(trifluoromethyl)-1-((2-(trimethylsilyl)ethoxy)methyl)-1H-imidazole

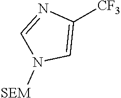 [SMILES: C[Si](C)(C)CCOCn1cnc(C(F)(F)F)c1]

To a mixture of 4-(trifluoromethyl)-1H-imidazole (5.4 g) and Cs₂CO₃ (13.2 g) in DMF (100 mL) was added 2-(trimethylsilyl)ethoxymethyl chloride (8.3 g). The reaction mixture was stirred at 80° C. for 4 h, then was poured into 100 mL ice-water, extracted with EtOAc (3×50 mL). The combined organic layer was dried over anhydrous Na₂SO₄, concentrated, and purified by SGCC (PE/EtOAc: from 100:0 to 10:1) to afford the title compound as a white solid (7.5 g). LCMS (ESI): 267.3 [M+1]⁺.

### Step 2 2-bromo-4-(trifluoromethyl)-1-((2-(trimethylsilyl)ethoxy)methyl)-1H-imidazole

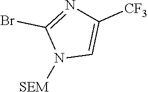 [SMILES: C[Si](C)(C)CCOCn1cc(C(F)(F)F)nc1Br]

To a stirred solution of the product of Step 1 (5.32 g) in THE (50 mL) at −78° C., was added n-butyllithium (2.5 M in hexane, 1.3 mL) dropwise. The resulting mixture was stirred for 30 min at −78° C. and then treated with a solution of carbon tetrabromide (6.6 g) in dry THF (20 mL). The reaction mixture was allowed to warm to rt and then stirred for 2 h. The reaction mixture was then quenched with saturated ammonium chloride solution and extracted with EtOAc (3×100 mL). The combined organic layer was dried over anhydrous Na₂SO₄, filtered and concentrated under reduced pressure. The residue was purified by SGCC eluted with 0-10% EtOAc in hexane to afford the title compound (4.6 g). LCMS (ESI): 346.3 [M+1]⁺.

### Step 3 methyl 4-bromo-3-(3-((tert-butyldimethylsilyl)oxy)propoxy)benzoate

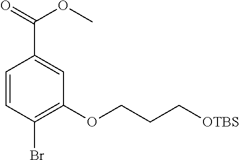 [SMILES: COC(=O)c1ccc(Br)c(OCCCO[Si](C)(C)C(C)(C)C)c1]

A mixture of methyl 4-bromo-3-hydroxybenzoate (1.0 g), (3-bromopropoxy)(tert-butyl) dimethylsilane (1.3 g) and Cs₂CO₃ (2.8 g) in DMF (20 mL) was stirred for 3 h at 60° C. The mixture was then treated with ammonium chloride (1N, 100 mL) and extracted with EtOAc (3×50 mL). The combined organic layer was dried over anhydrous Na₂SO₄ and concentrated. The residue was purified by SGCC (PE/EtOAc: from 100:0 to 2:1) to afford the title compound (1.6 g) as a yellow oil. LCMS (ESI): 404.3 [M+1]⁺.

### Step 4 (4-bromo-3-(3-((tert-butyldimethylsilyl)oxy)propoxy)phenyl)methanol

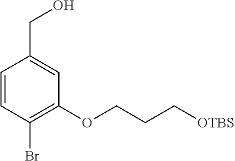 [SMILES: CC(C)(C)[Si](C)(C)OCCCOc1cc(CO)ccc1Br]

To a solution of the product of Step 3 (1.6 g) in THF (20 mL) was added dropwise DIBAL-H (10 mL, 1M in hexane) at 0° C. The reaction solution was stirred at 0° C. for 1 h and then quenched by MeOH, followed by addition of potassium sodium tartrate solution. After stirring at r.t. for 30 min, the reaction mixture was extracted with EtOAc (3×60 mL). The combined organic layer was dried over anhydrous Na₂SO₄ and concentrated. The residue was purified by SGCC (PE/EtOAc: from 100:0 to 2:1) to afford the title compound (1.5 g) as a colorless oil. LCMS (ESI): 376.4 [M+1]⁺.

### Step 5 1-(4-bromo-3-(3-((tert-butyldimethylsilyl)oxy)propoxy)benzyl)-6-(4-cyclopropyl-6-methoxypyrimidin-5-yl)-1H-pyrazolo[3,4-d]pyrimidine

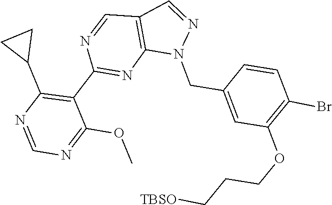 [SMILES: COc1ncnc(C2CC2)c1-c1ncc2cnn(Cc3ccc(Br)c(OCCCO[Si](C)(C)C(C)(C)C)c3)c2n1]

To a solution of the product of Step 4 (1.5 g), the product of Step 9 (0.91 g) of EXAMPLE 3 and n-Bu₃P (1.03 g) in THE (40 mL) was added dropwise DIAD (0.89 g) at r.t. After stirring at r.t. for 1 h, the reaction solution was concentrated and the residue was purified by SGCC eluted with EtOAc and PE (0 to 80% EtOAc) to afford the title product (1.8 g) as a yellow solid. LCMS (ESI): 626.6 [M+1]⁺.

### Step 6 3-(2-bromo-5-((6-(4-cyclopropyl-6-methoxypyrimidin-5-yl)-1H-pyrazolo[3,4-d]pyrimidin-1-yl)methyl)phenoxy)propan-1-ol

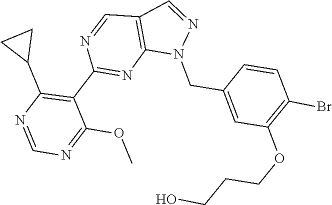 [SMILES: COc1ncnc(C2CC2)c1-c1ncc2cnn(Cc3ccc(Br)c(OCCCO)c3)c2n1]

A solution of the product of Step 5 (1.8 g) in 1,4-dioxane/3M aq. HCl (5/5 mL) was stirred at rt for 0.5 h. The reaction mixture was adjusted pH to 7 with K₂CO₃ (aq.) and extracted with EA (3×50 mL). The combined organic layer was washed with brine (3×30 mL) and concentrated to obtain the title product as white solid (1.5 g). LCMS (ESI): 512.3 [M+1]⁺.

### Step 7 4⁴-bromo-1⁶-cyclopropyl-2′H-5,9-dioxa-2(6,1)-pyrazolo[3,4-d]pyrimidina-1(5,4)-pyrimidina-4(1,3)-benzenacyclononaphane

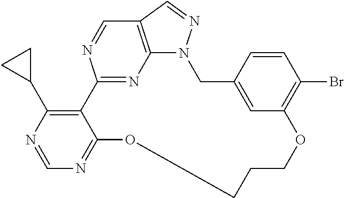 [SMILES: Brc1ccc2cc1OCCCOc1ncnc(C3CC3)c1-c1ncc3cnn(c3n1)C2]

A solution of the product of Step 6 (1.5 g), sodium hydride (0.3 g), 1,4,7,10,13-pentaoxacyclopentadecane (1.5 g) in 1,4-dioxane (75 mL) was stirred at 95° C. for 5 h. The reaction mixture was then cooled to rt, quenched with acetic acid (5 mL). After adjusting pH to 7 with K₂CO₃ (aq.), the mixture was extracted with EA (3×50 mL). The combined organic layer was dried over Na₂SO₄, filtered and concentrated. The residue was purified by SGCC (PE/EtOAc: from 100:0 to 3:1) to obtain the product as a yellow solid (1.2 g). LCMS (ESI): 480.3 [M+1]⁺.

### Step 8 1⁶-cyclopropyl-4⁴-(4,4,5,5-tetramethyl-1,3,2-dioxaborolan-2-yl)-2¹H-5,9-dioxa-2(6,1)-pyrazolo [3,4-d]pyrimidina-1(5,4)-pyrimidina-4(1,3)-benzenacyclononaphane

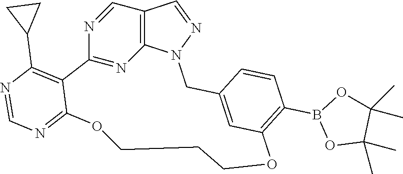 [SMILES: CC1(C)OB(c2ccc3cc2OCCCOc2ncnc(C4CC4)c2-c2ncc4cnn(c4n2)C3)OC1(C)C]

A mixture of the product of Step 7 (1.2 g), 4,4,4′,4′,5,5,5′,5′-octamethyl-2,2′-bi(1,3,2-dioxaborolane) (2.0 g), KOAc (1.2 g), Pd(dppf)Cl₂ (0.2 g) in 1,4-dioxane (50 mL) was stirred at 90° C. overnight under Ar. The reaction mixture was poured into water and extracted with EtOAc (3×60 mL). The combined organic layer was dried over Na₂SO₄, filtered and concentrated. The residue was purified by SGCC (PE/EtOAc: from 100:0 to 3:1) to afford product as a pale yellow solid (1.3 g). LCMS (ESI): 527.4 [M+1]⁺.

### Step 9 1⁶-cyclopropyl-4⁴-(5-methyl-3-(trifluoromethyl)-1H-pyrazol-1-yl)-2¹H-5,9-dioxa-2(6,1)-pyrazolo[3,4-d]pyrimidina-1(5,4)-pyrimidina-4(1,3)-benzenacyclononaphane

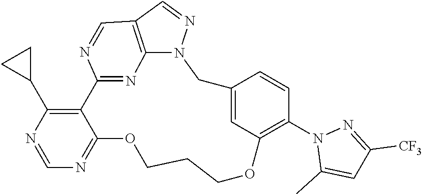 [SMILES: Cc1cc(C(F)(F)F)nn1-c1ccc2cc1OCCCOc1ncnc(C3CC3)c1-c1ncc3cnn(c3n1)C2]

A mixture of the product of Step 8 (0.25 g), 5-methyl-3-(trifluoromethyl)-1H-pyrazole (0.38 g), Cu(OAc)₂ (109 mg), boric acid (60 mg) and pyridine (50 mg) in CH₃CN (10 mL) was stirred for 16 h at 50° C. The reaction mixture was concentrated and the residue was purified by SGCC (PE/EtOAc: from 100:0 to 3:1) to afford the title compound as a white solid (65 mg). LCMS (ESI): 549.5 [M+1]⁺.

### Example 57

### ((E)-1⁶-cyclopropyl-4⁴-(1-cyclopropyl-4-(trifluoromethyl)-1H-imidazol-2-yl)-2¹H-8-oxa-2(6,1)-pyrazolo[3,4-d]pyrimidina-1(5,4)-pyrimidina-4(1,2)-benzenacyclooctaphan-5-ene

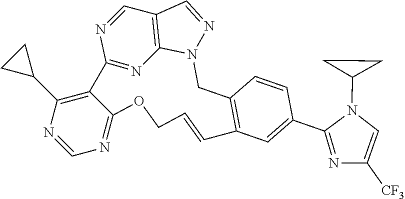 [SMILES: FC(F)(F)c1cn(C2CC2)c(-c2ccc3c(c2)/C=C/COc2ncnc(C4CC4)c2-c2ncc4cnn(c4n2)C3)n1]

### Step 1 5-(1-cyclopropyl-4-(trifluoromethyl)-1H-imidazol-2-yl)-2-((6-(4-cyclopropyl-6-methoxypyrimidin-5-yl)-1H-pyrazolo[3,4-d]pyrimidin-1-yl)methyl)phenyl trifluoromethanesulfonate

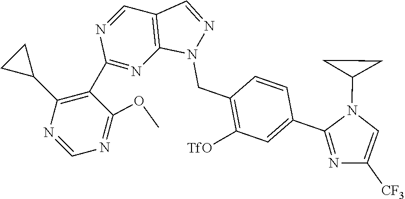 [SMILES: COc1ncnc(C2CC2)c1-c1ncc2cnn(Cc3ccc(-c4nc(C(F)(F)F)cn4C4CC4)cc3OS(=O)(=O)C(F)(F)F)c2n1]

Starting with 5-(1-cyclopropyl-4-(trifluoromethyl)-1H-imidazol-2-yl)-2-((6-(4-cyclopropyl-6-methoxypyrimidin-5-yl)-1H-pyrazolo [3,4-d]pyrimidin-1-yl)methyl)phenol, the title product was obtained as a white solid by following the same procedures as described in Step 1 of EXAMPLE 32. LCMS (ESI): 681.2 [M+1]⁺.

### Step 2 ((E)-1-(2-(3-((tert-butyldimethylsilyl)oxy)prop-1-en-1-yl)-4-(1-cyclopropyl-4-(trifluoromethyl)-1H-imidazol-2-yl)benzyl)-6-(4-cyclopropyl-6-methoxypyrimidin-5-yl)-1H-pyrazolo[3,4-d]pyrimidine

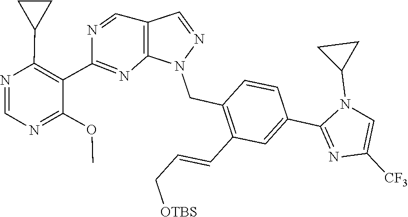 [SMILES: COc1ncnc(C2CC2)c1-c1ncc2cnn(Cc3ccc(-c4nc(C(F)(F)F)cn4C4CC4)cc3/C=C/CO[Si](C)(C)C(C)(C)C)c2n1]

To a mixture of the product of Step 1 (200 mg) and ethyl (E)-tert-butyldimethyl((3-(4,4,5,5-tetramethyl-1,3,2-dioxaborolan-2-yl)allyl)oxy)silane (1.25 g) in 1,4-dioxane/water (20/2 mL) were added Pd₂(dba)₃ (67.0 mg) and 2-(dicyclohexylphosphino)-2,4,6-triisopropylbiphenyl (70.0 mg) and CsF (170 mg). The reaction mixture was then stirred at 100° C. under nitrogen for 18 h. The reaction mixture was then poured into 100 mL ice-water, extracted with EtOAc (3×20 mL). The combined organic layer was washed with brine (20 mL), dried over anhydrous Na₂SO₄ and concentrated. The residue was purified using SGCC (PE/EtOAc: from 100:0 to 10:1) to afford the title compound as a yellow solid (200 mg). LCMS (ESI): 703.3 [M+1]⁺.

### Step 3 (E)-6-cyclopropyl-5-(1-(4-(1-cyclopropyl-4-(trifluoromethyl)-1H-imidazol-2-yl)-2-(3-hydroxyprop-1-en-1-yl)benzyl)-1H-pyrazolo[3,4-d]pyrimidin-6-yl)pyrimidin-4-ol

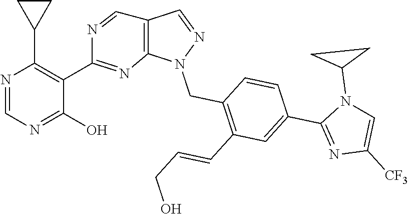 [SMILES: OC/C=C/c1cc(-c2nc(C(F)(F)F)cn2C2CC2)ccc1Cn1ncc2cnc(-c3c(O)ncnc3C3CC3)nc21]

Starting with the product of Step 2, the title product was obtained as a white solid by following the same procedures as described in Step 13 of EXAMPLE 3. LCMS (ESI): 575.2 [M+1]⁺.

### Step 4 ((E)-1⁶-cyclopropyl-4⁴-(1-cyclopropyl-4-(trifluoromethyl)-1H-imidazol-2-yl)-2¹H-8-oxa-2(6,1)-pyrazolo[3,4-d]pyrimidina-1(5,4)-pyrimidina-4(1,2)-benzenacyclooctaphan-5-ene

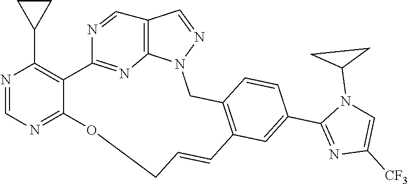 [SMILES: FC(F)(F)c1cn(C2CC2)c(-c2ccc3c(c2)/C=C/COc2ncnc(C4CC4)c2-c2ncc4cnn(c4n2)C3)n1]

Starting with the product of Step 3, the title product was obtained as a white solid by following the same procedures as described in Step 14 of EXAMPLE 3. LCMS (ESI): 557.2 [M+1]⁺.

1. A compound of Formula I or a pharmaceutically acceptable salt thereof: 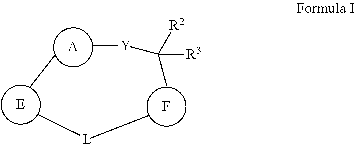
Wherein
Ring A is selected from the following structures: 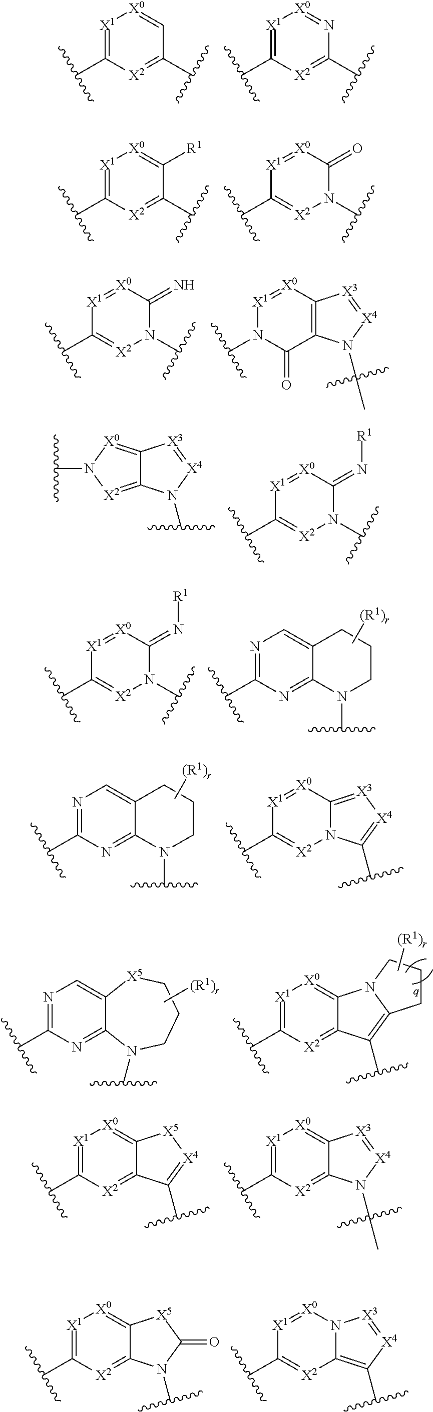 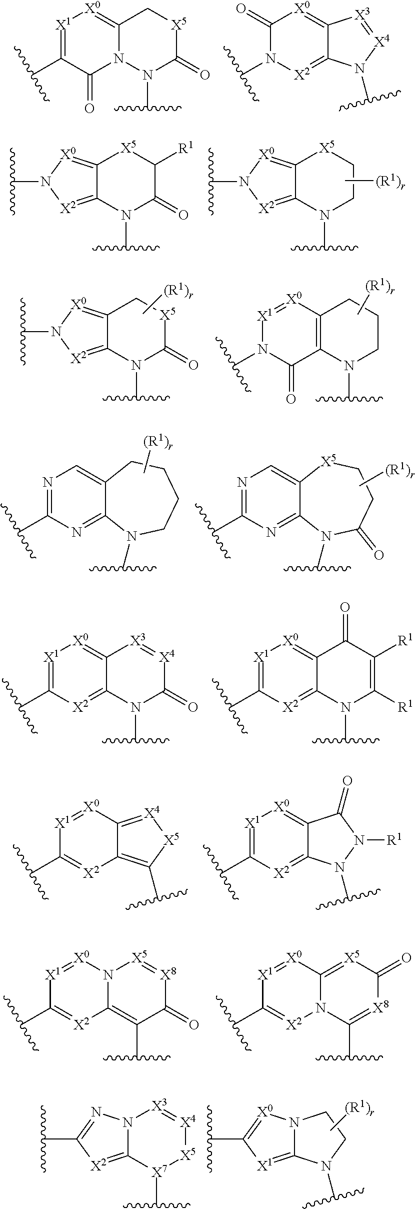 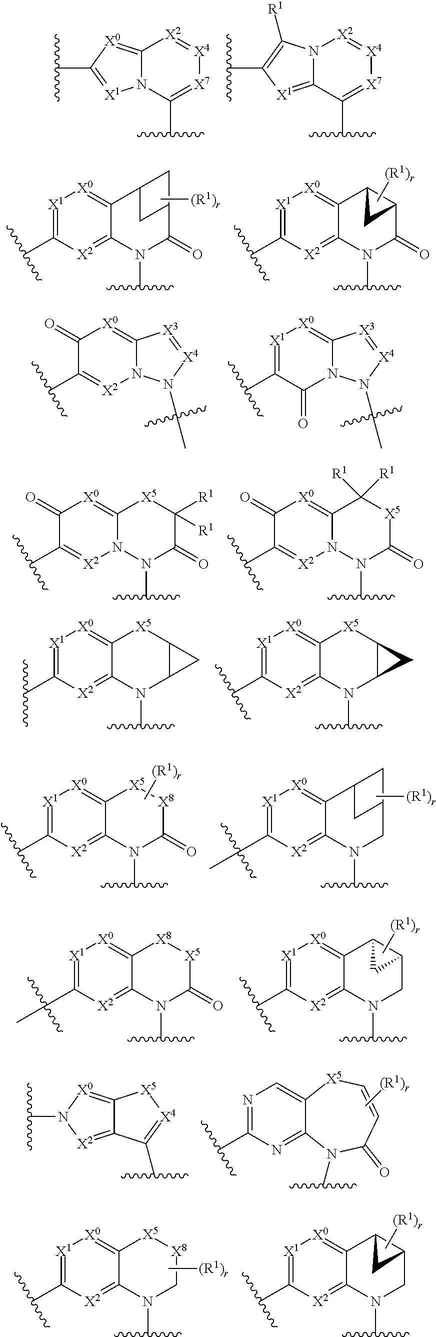 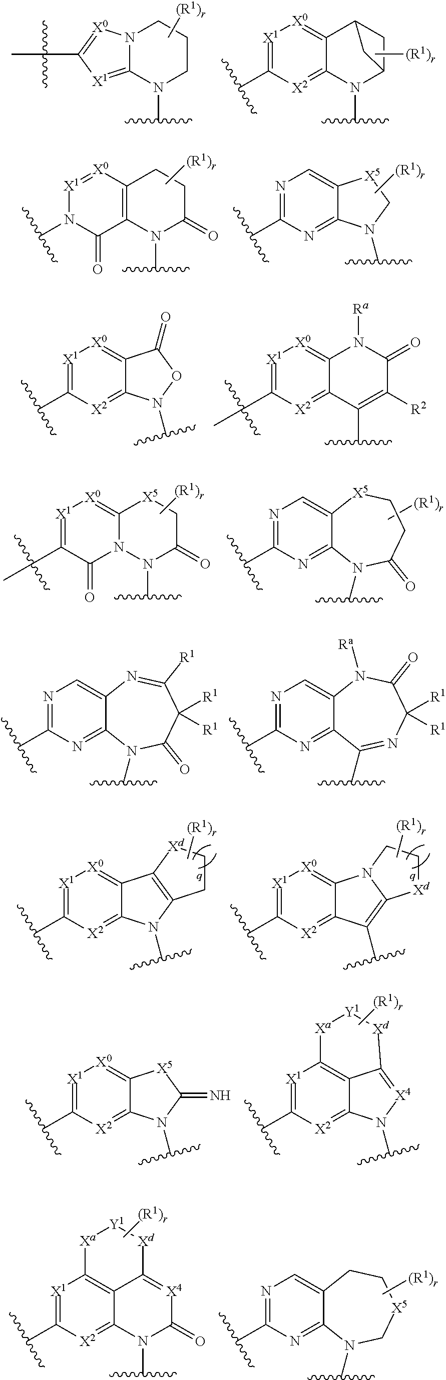 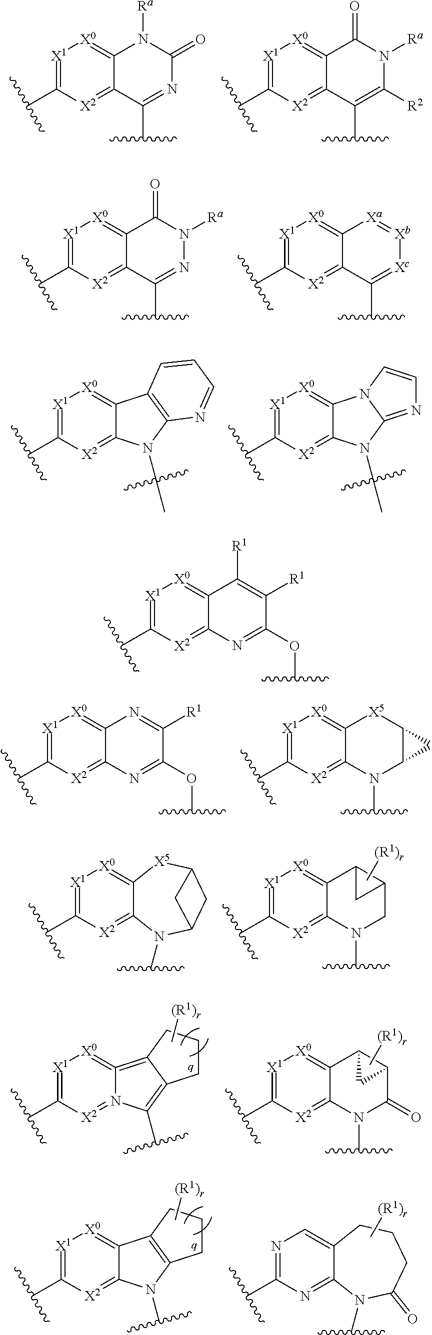 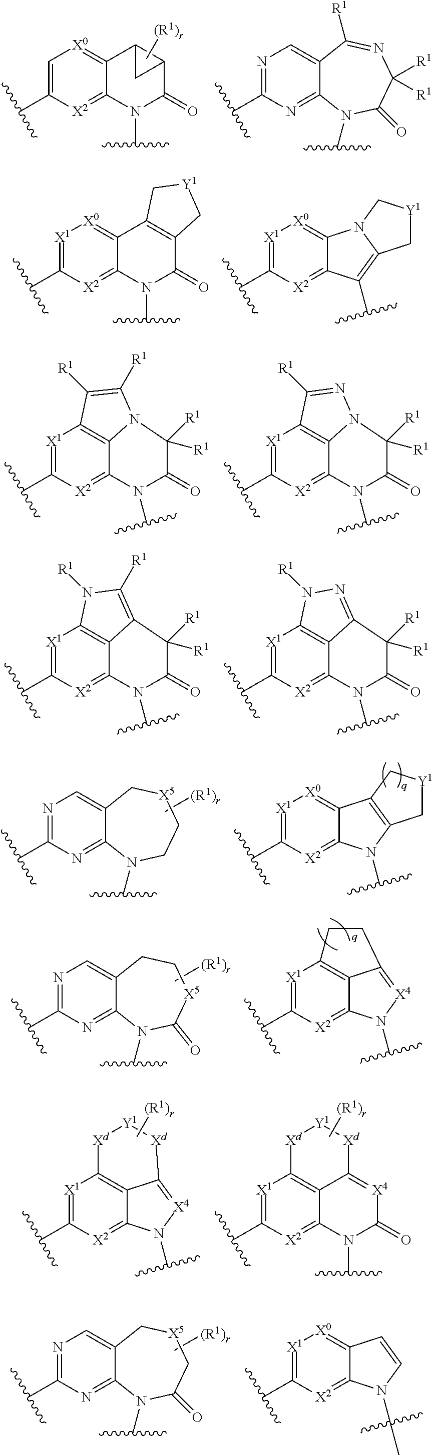 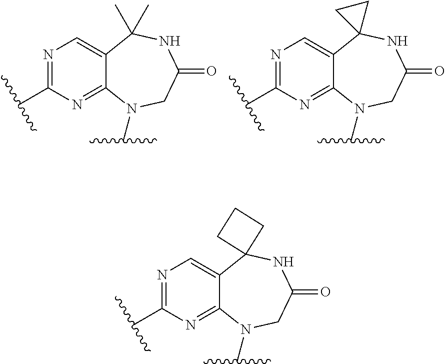 [SMILES: *c1ncc2c(n1)N(*)CC(=O)NC2(C)C ; *c1ncc2c(n1)N(*)CC(=O)NC21CC1 ; *c1ncc2c(n1)N(*)CC(=O)NC21CCC1]
Y and Y¹ are independently a chemical bond or selected from NR¹, O, S, S(O)₂ or C(R¹)₂;
Each of X⁰, X¹, X², X³, X⁴, X^{a}, X^{b} and X^{c} is independently selected from N or CR¹;
X⁵ and X⁸ are independently selected from C(R¹), C(R¹)₂, O, S or NR¹;
r and q are independently 0, 1, 2, 3 or 4;
X⁶ is NR^{b}, C═O, CR^{a}R^{b};
X⁷ is N or CR¹;
X^{d} is independently a chemical bond or selected from CH₂, C(O), O, S, S(O)₂, O or NR^{d};
R¹ is independently selected from the group consisting of hydrogen, halogen, C₁-C₆ alkyl, C₁-C₆ halogenated alkyl, C₁-C₆ alkoxy, C₁₋₆ alkylthio, hydroxyl, nitro, formyl, —CN, —SF₅, —NR^{a}R^{b}, carboxyl, —C(O)R^{a}, —C(O)₂C₁-C₆ alkyl, —C(O)NR^{a}R^{b}, —S(O)₂R^{c}, —S(O)₂NR^{a}R^{b}, —P(O)Me₂ and —P(O)(OMe)₂;
Two R¹ on the same carbon atom or adjacent carbon atoms together with carbon atom(s) can form an oxo group or a three- to six-membered carbocyclic ring, four- to eight-membered bicyclic ring or five- to eight-membered spirocyclic ring which is optionally substituted with R^{e} or a three- to six-membered carbocyclic ring, four- to eight-membered bicyclic ring or five- to eight-membered spirocyclic ring which contains one, two or three heteroatom(s) such as S, O or NR^{d};
R^{a} and R^{b} are independently hydrogen, CH₂CH₂OH, CH₂CMe₂OH, C₁-C₆ alkanol, C₁-C₆ halogenated alkanol, substituted/non-substituted C₁-C₁₀ alkyl, substituted/non-substituted C₃-C₁₀ cycloalkyl, substituted/non-substituted C₂-C₁₀ alkenyl, substituted/non-substituted C₆-C₂₀ aryl, or substituted/non-substituted C₃-C₁₄ heteroaryl respectively; R^{a} and R^{b} can form 3-8 membered carbocyclic rings or 4-8 membered heterocyclic rings, which contain sulfur, oxygen, NH or NR^{d};
R^{c} represents C₁-C₁₀ alkyl, C₃-C₁₀ cycloalkyl, C₆-C₂₀ aryl, or C₃-C₁₄ heteroaryl; R^{c} can be substituted by one or more radical groups selected from halogen, hydroxyl, amino, nitro, cyano, formyl, carboxyl, alkoxy, —CF₃ and —SF₅;
R^{d} is selected from the group consisting of hydrogen, C₁₋₆alkyl, C₃₋₆cyclolkyl, C₃₋₆fluorocycloalkyl, C₁₋₆fluoroalkyl, C₀₋₆alkylene-aryl, C₀₋₆alkylene-heteroaryl, C(O)C₁₋₆alkyl, C(O)NR^{a}R^{b}, C(O)aryl, S(O)₂C₁₋₆alkyl, S(O)₂C₃₋₆cycloalkyl, S(O)₂ary, S(O)₂NR^{a}R^{b};
R^{e} is independently selected from the group consisting of hydrogen, halogen, C₁-C₆ alkyl, C₁-C₆ halogenated alkyl, C₁-C₆ alkoxy, nitro, formyl, —CN, —SF₅, NH₂, C₁-C₁₀ alkyl, C₃-C₁₀ cycloalkyl, C₆-C₂₀ aryl, or C₃-C₁₄ heteroaryl;
Each of R² and R³ is independently selected from the group consisting of H, NR^{a}R^{b}, C₁-C₆ alkoxy, C₁₋₆ alkylthio, halogen, CN, C₁₋₆alkyl, C₃-C₆cyclolkyl, C₃-C₆fluorocycloalkyl, C₁-C₆fluoroalkyl, C₂-C₆ alkenyl, C₂-C₆ alkynyl, wherein the alkyl, alkenyl, alkynyl and cycloalkyl are unsubstituted or substituted with one or more substituents selected from halogen, C₁-C₆alkyl, C₁-C₆halogenated alkyl, C₃-C₁₀cycloalkyl, C₃-C₁₀ cycloalkoxy, C₁-C₆alkoxy, hydroxyl, nitro, formyl, —CN, —SF₅, —NR^{a}R^{b}, carboxyl, —C(O)R^{a}, —C(O)₂C₁-C₆alkyl, —C(O)NR^{a}R^{b}, —S(O)₂R^{c}, —S(O)₂NR^{a}R^{b}, —P(O)Me₂ and —P(O)(OMe)₂; R² and R³, together with the carbon atom to which they are both attached to, complete a three- to six-membered carbocyclic ring, four- to ten-membered bicyclic ring or five- to ten-membered spirocyclic ring which is optionally substituted with R^{d} or a three- to six-membered carbocyclic ring, four- to eight-membered bicyclic ring or five- to eight-membered spirocyclic ring which contains one, two or three heteroatom(s) such as S, O or NR^{d};
Ring E represents C₆-C₂₀ aromatic ring or C₁-C₂₀ heteroaromatic ring substituted by one or more groups selected from halogen, C₁-C₆ alkyl, C₁-C₆ alkanol, C₁-C₆ halogenated alkyl, C₃-C₁₀ cycloalkyl, C₃-C₁₀ cycloalkoxy, C₁-C₆ alkoxy, hydroxyl, nitro, formyl, —CN, —SF₅, NR^{a}R^{b}, carboxyl, —C(O)R^{a}, —C(O)₂C₁-C₆ alkyl, —C(O)NR^{a}R^{b}, —SO₂R^{c}, —S(O)₂NR^{a}R^{b}, —P(O)Me₂ and —P(O)(OMe)₂; 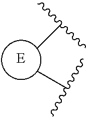
is selected from the following structures: 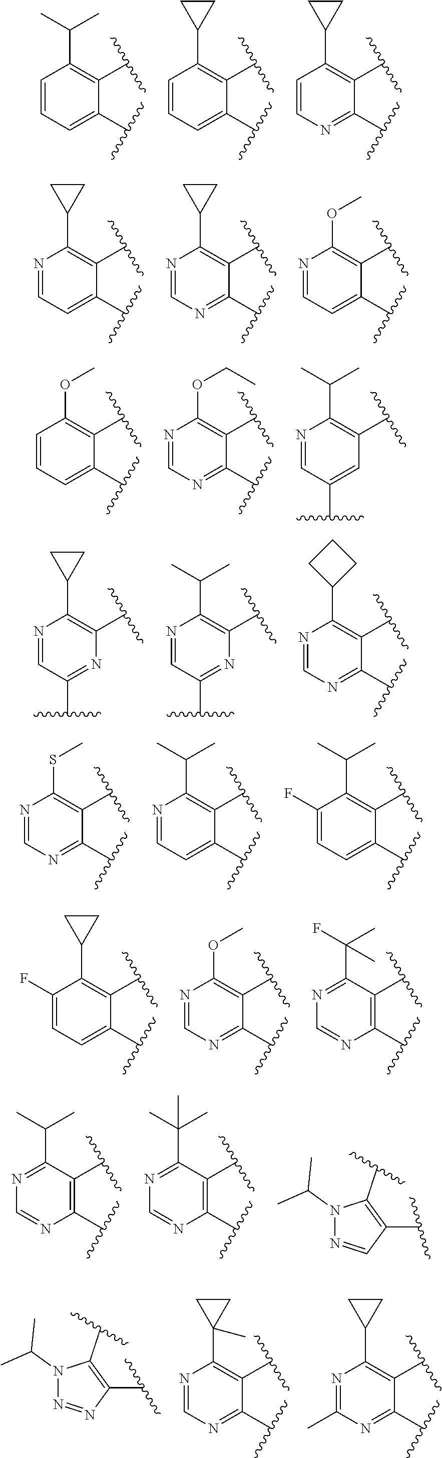 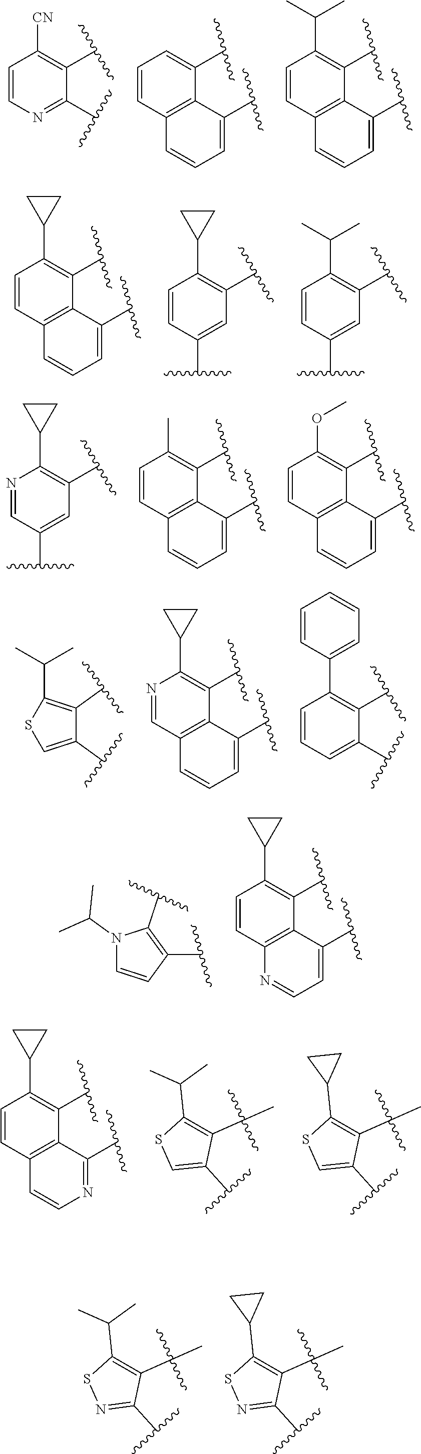 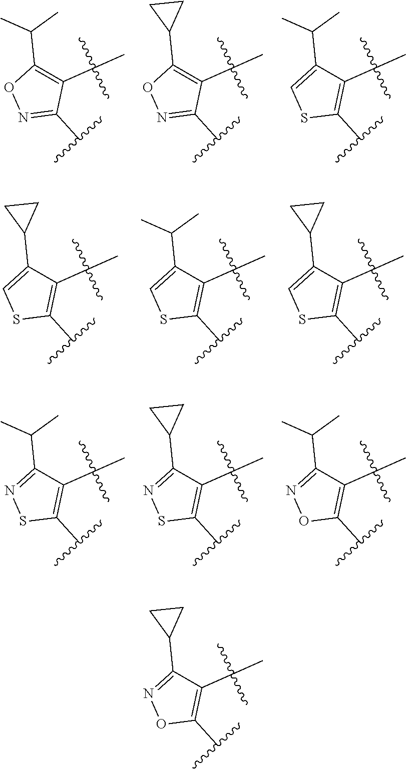
Ring F represents C₆-C₁₄aromatic ring, C₁-C₂₀ heteroaromatic ring, C₃-C₁₄ carbocyclic ring, C₄-C₁₄bicarbocyclic ring, C₅-C₁₄spirocarbocyclic ring, C₄-C₁₄heterocyclic ring, C₄-C₁₄heterobicyclic ring, C₅-C₁₄heterotricyclic ring, C₅-C₁₄heterospirocyclic ring with 0 to 5 of O, N or S atoms independently; Ring F is substituted with one or more R⁴;
Preferably 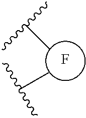
is selected from the following structures: 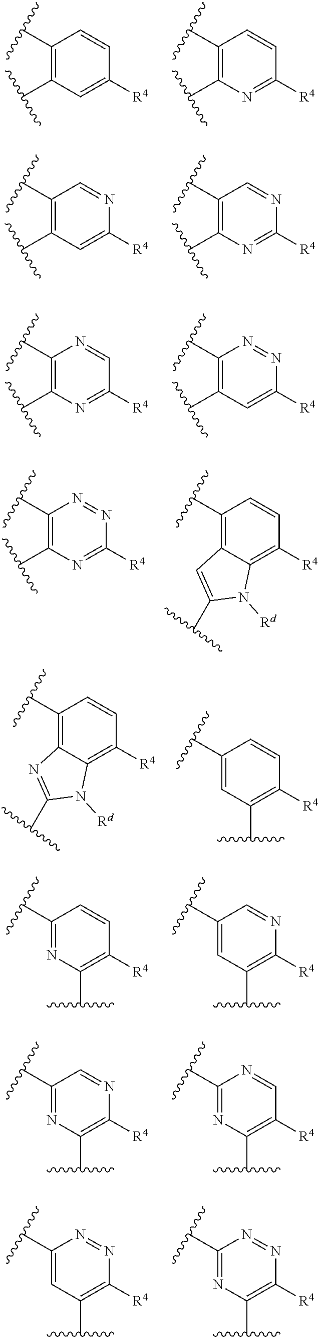 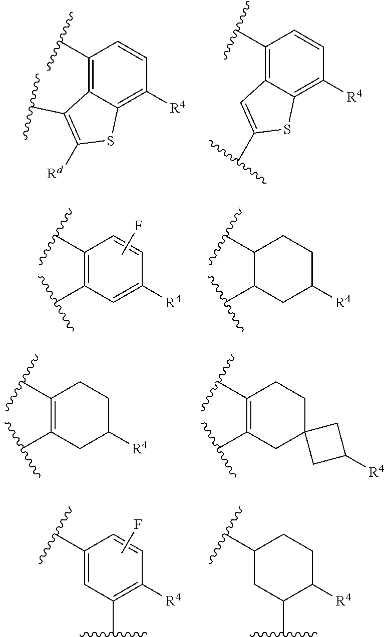 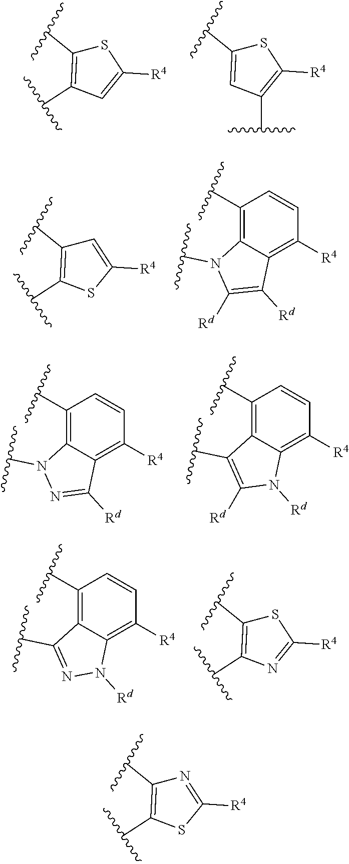
R⁴ represents unsubstituted or substituted C₁-C₆alkyl, unsubstituted or substituted C₂-C₆alkenyl, unsubstituted or substituted C₂-C₆alkynyl, unsubstituted or substituted C₁-C₆alkoxy, C₁-C₆haloalkyl, C₁-C₆haloalkoxy, C₁-C₆hydroxyalkyl, CN, halogen, S(O)₂NH₂, —C(O)R^{a}, —C(O)₂R^{a}, —NR^{b}R^{b}, —NR^{a}C(O)R^{b}, —NR^{a}C(O)NR^{a}R^{a}, —C(O)NR^{a}R^{b}, —S(O)₂NR^{a}R^{b}, —NR^{a}S(O)₂R^{b}, optionally C₆-C₁₀ aryl and C₁-C₁₀heteroaryl; R⁴ is unsubstituted or substituted by one or more groups selected from: halogen, C₁-C₆alkyl, C₃-C₆cycloalkyl, C₃-C₆ halogenated cycloalkyl, C₁-C₆halogenated alkyl, C₁-C₆alkoxy, hydroxyl, nitro, formyl, —CN, —SF₅, NR^{a}R^{b}, carboxyl, —C(O)R^{a}, —C(O)₂C₁-C₆ alkyl, —C(O)NR^{a}R^{b}, —S(O)₂R^{c}, —S(O)²NR^{a}R^{b}, —P(O)Me₂ and —P(O)(OMe)₂; preferably, R⁴ is selected from the following structures: 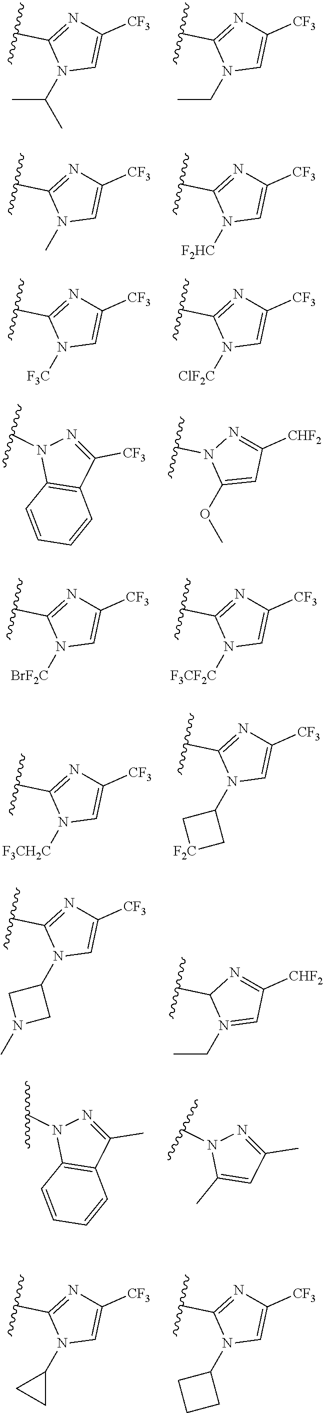 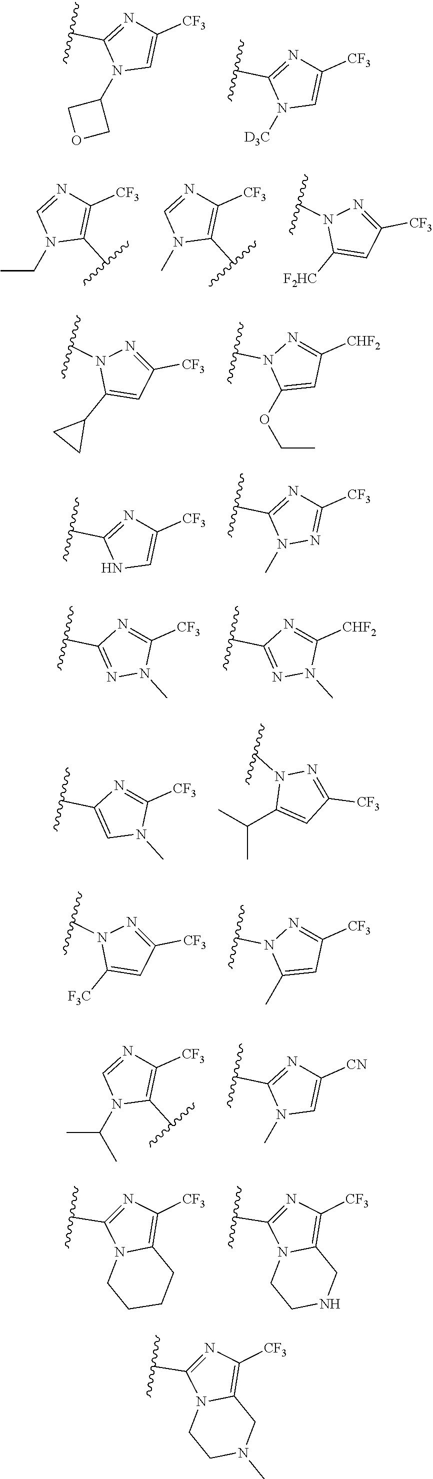
L is selected from —(CH₂)ₚ—, —O(CH₂)ₚ, —(CH₂)ₚ, S(O)ᵥ(CH₂)ₚ—, —S(O)₂(NR⁵)(CH₂)ₚ—, —(CH₂)ₚS(O)ᵥ—, —(CH₂)ₚSO₂(NR⁵)—, —NR⁵(CH₂)ₚ—, —(CH₂)ₚNR⁵—, —(CH₂)ₚO(CH₂)ᵣ—, —(CH₂)ₚNR⁵(CH₂)ᵣ—, —(CH₂)ₚS(O)ᵥ(CH₂)ᵣ—, —(CH₂)ₚ SO₂(NR⁵)(CH₂)ᵣ—, —(CH₂)ₚS(CH₂)ᵣ—, —O(CH₂)ₚO—, —NR⁵(CH₂)ₚNR⁶—, S(O)ᵥ(CH₂)ₚC(O)N—, —(CH₂)ₚS(O)ᵥ, —S(O)ᵥ(CH₂)ₚR—, —O(CH₂)ₚNR⁵—, —NR⁵(CH₂)ₚO—, —S(O)ᵥ(CH₂)ₚNR⁵—, —NR⁵(CH₂)ₚS(O)ᵥ—, O(CH₂)ₚSO(NR⁵)—, —SO(NR⁵)(CH₂)ₚO—, —SO(NR⁵)(CH₂)ₚNR⁶—, —NR⁵(CH₂)ₚSO(NR⁶)—, —(CH₂)ₚCF₂(CH₂)ᵣ—, —(CH₂)ₚCHF(CH₂)ᵣ—, —(CF₂)₄—, —(CF₂)₅—, —(CH₂)ₚC(O)—, —O(CH₂)ₚC(O)—, —(CH₂)ₚC(O)NR⁵—, —(CH₂)ₚC(S)NR⁵—, —(CH₂)ₚCH(CF₃)NR⁵—, —(CH₂)ₚNR⁵C(O)—, —(CH₂)ₚNR⁵CH(CF₃)—, —C(O)NR⁵(CH₂)ₚ—, —CH(CF₃)NR⁵(CH₂)ₚ—, —C(S)NR⁵(CH₂)ₚ—, —O(CH₂)ₚC(O)NR⁵—, —O(CH₂)ₚC(S)NR⁵—, —S(O)ᵥ(CH₂)ₚC(O)—, —O(CH₂)ₚC(O)NR⁵—, —NR⁵C(O)(CH₂)ₚC(O)NR⁶—, —C(O)NR⁵(CH₂)ₚC(O)NR⁶—, —C(O)NR⁵(CH₂)ₚNR⁶C(O)—, —NR⁵C(O)(CH₂)ₚNR⁶C(O)—, —S(O)ᵥ(CH₂)ₚO—, —O(CH₂)ₚS(O)ᵥ—, —S(O)ᵥ(CH₂)ₚ—, —(CH₂)ₚS(O)ᵥ—, —S(O)ᵥ(CH₂)ₚS(O)ᵥ—, —NR⁵(CH₂)ₚC(O)NR⁶—, —NR⁵C(O)(CH₂)ₚ—, —NR⁵CH(CF₃)(CH₂)ₚ—, —NR⁵(CH₂)ₚO—, —(CH₂)ᵣOC(O)(CH₂)ₚ—, —OC(O)(CH₂)ₚ—, —OC(O)(CH₂)ₚS(O)ᵥ—, —(CH₂)ₚCR^{aa}═CR^{bb}(CH₂)ᵣ—, —NR⁵(CH₂)ₚCR^{aa}═CR^{bb}(CH₂)ᵣ—, —NR⁵C(O)(CH₂)ₚCR^{aa}═CR^{bb} (CH₂)ᵣ—, —(CH₂)ₚCR^{aa}═CR^{bb}(CH₂)ᵣC(O)NR⁵—, —(CH₂)ₚNR⁵C(O)NR⁶(CH₂)ᵣ—, —(CH₂)ₚNR⁵C(S)NR⁶(CH₂)ᵣ—, —(CH₂)ₚNR⁵S(O)₂NR⁶(CH₂)ᵣ—, —(CH₂)ₚS(O)ᵥ(CH₂)ᵣ—, —(CH₂)ₚS(O)₂NR⁵(CH₂)ᵣ—, —(CH₂)ₚNR⁵(O)ᵥ(CH₂)ᵣ—, —(CH₂)ₚS(CH₂)ᵣ—, —(CH₂)ₚC≡C(CH₂)ᵣ—, —O(CH₂)ₚCR^{aa}═CR^{bb}(CH₂)ᵣ—, —O(CH₂)ₚC≡C(CH₂)ᵣ—, —(CH₂)ₚCR^{aa}═CR^{bb} (CH₂)ᵣO—, —(CH₂)ₚC≡C(CH₂)ᵣO—, —O(CH₂)ₚCR^{aa}═CR^{bb} (CH₂)ᵣO—, —O(CH₂)ₚC≡C(CH₂)ᵣO—, —S(O)ᵥ(CH₂)ₚCR^{aa}═CR^{bb}(CH₂)ᵣ—, S(O)ᵥ(CH₂)ₚC≡C(CH₂)ᵣ—, —(CH₂)ₚCR^{aa}═CR^{bb} (CH₂)ᵣS(O)ᵥ—, —(CH₂)ₚC≡C(CH₂)ᵣS(O)ᵥ—, —O(CH₂)ₚCR^{aa}═CR^{bb}(CH₂)ᵣS(O)ᵥ—, —O(CH₂)ₚC≡C(CH₂)ᵣS(O)ᵥ—, —S(O)ᵥ(CH₂)ₚCR^{aa}═CR^{bb}(CH₂)ᵣO—, —S(O)ᵥ(CH₂)ₚC≡C(CH₂)ᵣO—, —C(O)NR⁵S(O)₂(CH₂)ₚ—, or —(CH₂)ₚS(O)₂NR⁶C(O)—; or L is L₁-L₂-L₃;
L₁ and L₃ are independently selected from —(CH₂)ₚ—, —O(CH₂)ₚ—, —S(CH₂)ₚ—, —NR⁵(CH₂)ₚ—, —(CH₂)ₚNR⁵—, —(CH₂)ₚO—, —(CH₂)ₚS—, —(CH₂)ₚC(O)—, —C(O)(CH₂)ₚ—, —(CH₂)ₚC(O)NR⁵—, —NR⁵C(O)(CH₂)ₚ—, —(CH₂)ₚCR^{aa}═CR^{bb}(CH₂)ᵣ—, —O(CH₂)ₚCR^{aa}═CR^{bb}(CH₂)ᵣ—, —(CH₂)ₚCR^{aa}═CR^{bb}(CH₂)ᵣO—, —S(CH₂)ₚCR^{aa}═CR^{bb}(CH₂)ᵣ—, —(CH₂)ₚCR^{aa}═CR^{bb} (CH₂)ᵣS—, —O(CH₂)ₚCR^{aa}═CR^{bb} (CH₂)ᵣS—, or —S(CH₂)ₚCR^{aa}═CR^{bb} (CH₂)ᵣO—;
L₂ is a C₂₋₆heterocyclic ring, an C₆₋₁₀aryl ring or a C₁₋₉heteroaryl ring;
CR^{aa}═CR^{bb} (olefinic double bond) in L, L₁ and L₃ includes both E and Z geometric isomers;
R^{aa} and R^{bb} are independently selected from H, halogen, C₁₋₆alkyl, C₁₋₆halogenated alkyl, C₃₋₆ cycloalkyl, C₃₋₆halogenated cycloalkyl;
p is 1, 2, 3, 4, 5 or 6; v is 0, 1 or 2;
R⁵ and R⁶ are independently selected from hydrogen, C₁₋₆alkyl, C₃₋₆cycloalkyl.

2. A compound according to claim 1, or a pharmaceutically acceptable salt thereof, wherein the said compound is represented by Formula II to Formula XIX: 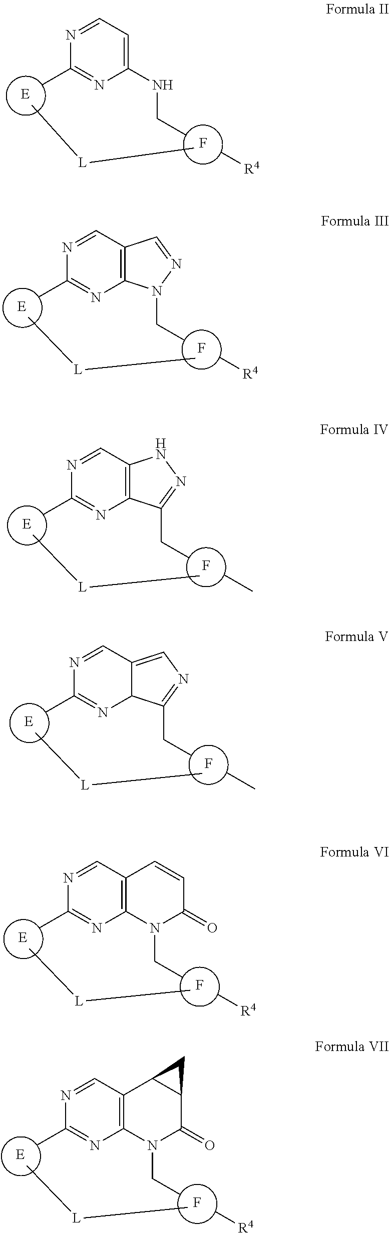 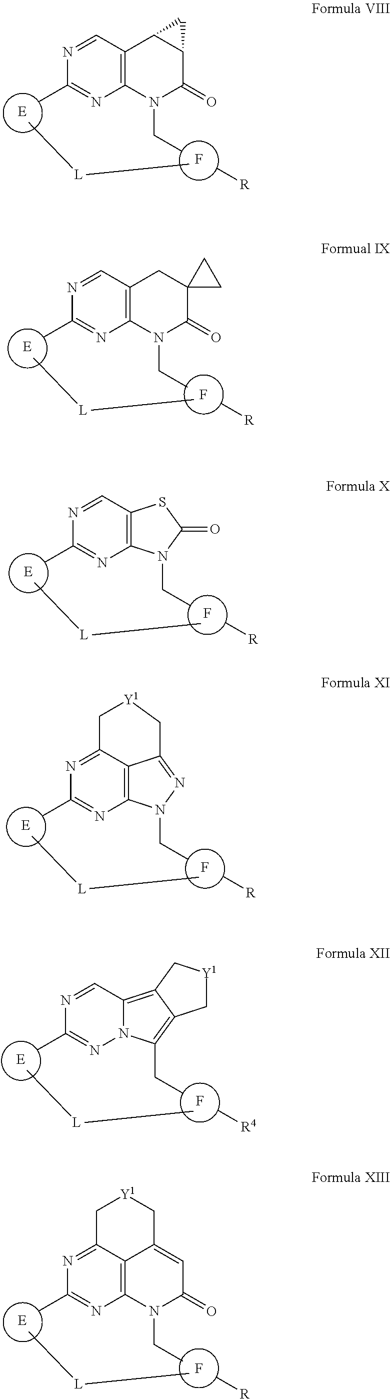 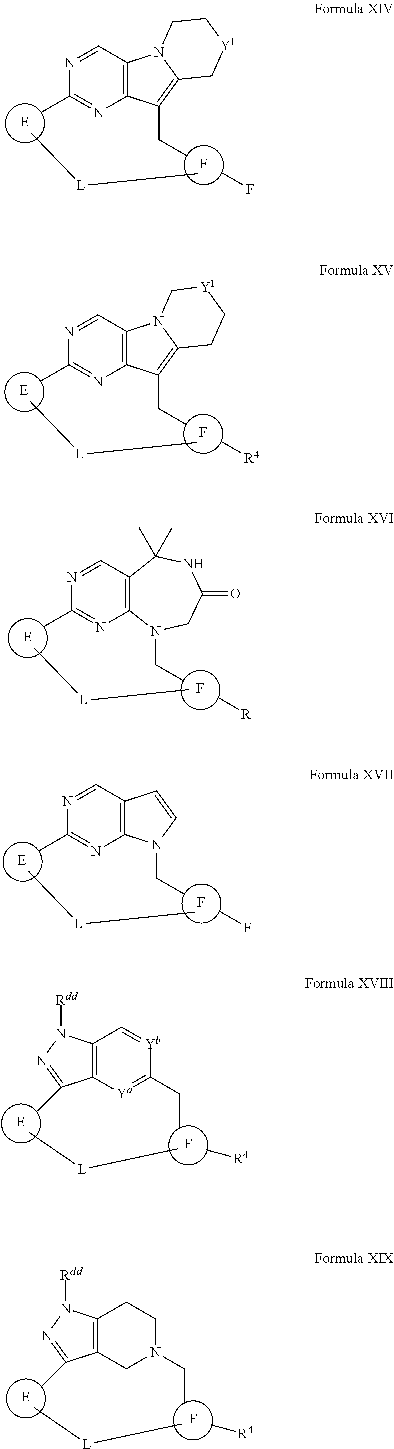
wherein ring E, ring F, L, Y¹ an R⁴ are defined as in Formula I. Y^{a} an Y^{b} are independently N, CR^{ee}. R^{dd} is selected from H, C₁₋₆alkyl C₃₋₆cycloalkyl. R^{ee} is selected from H, halogen, C₁₋₆alkyl, halogenated C₁₋₆alkyl, C₃₋₆cycloalkyl.

3. The compound of claim 1 wherein the compound is selected from:

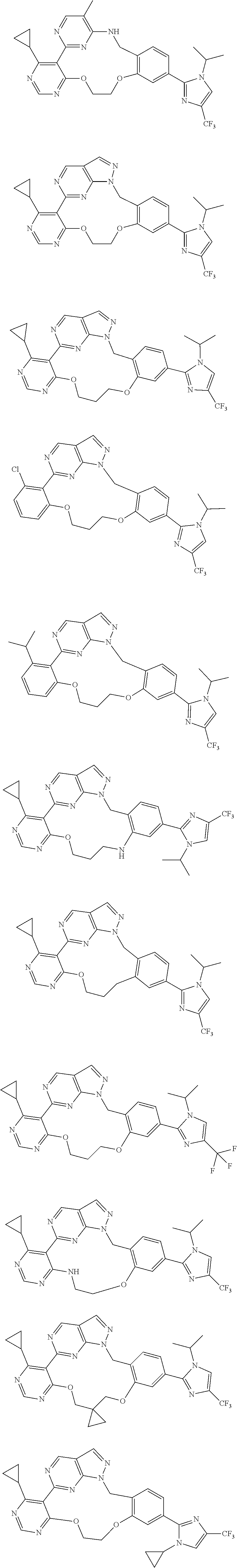

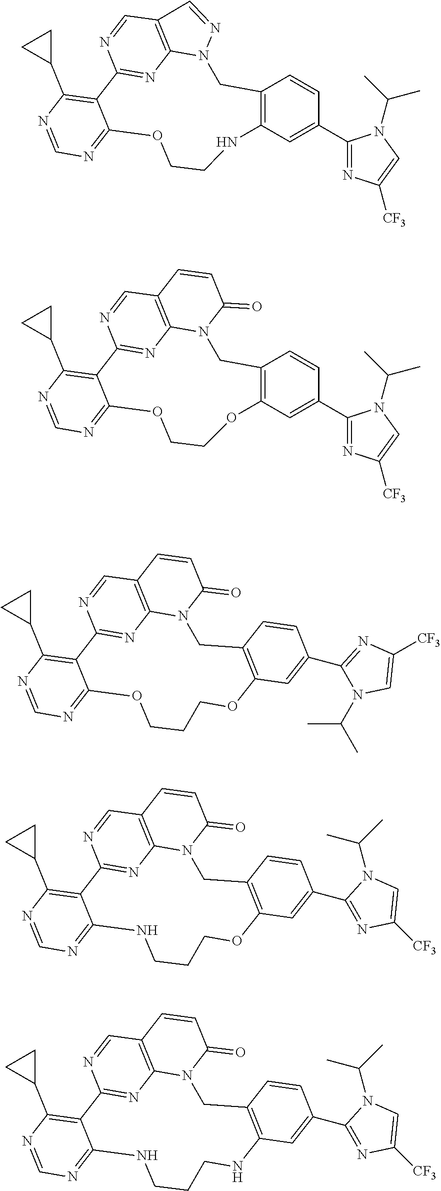 [SMILES: CC(C)n1cc(C(F)(F)F)nc1-c1ccc2c(c1)NCCOc1ncnc(C3CC3)c1-c1ncc3cnn(c3n1)C2 ; CC(C)n1cc(C(F)(F)F)nc1-c1ccc2c(c1)OCCOc1ncnc(C3CC3)c1-c1ncc3ccc(=O)n(c3n1)C2 ; CC(C)n1cc(C(F)(F)F)nc1-c1ccc2c(c1)OCCCOc1ncnc(C3CC3)c1-c1ncc3ccc(=O)n(c3n1)C2 ; CC(C)n1cc(C(F)(F)F)nc1-c1ccc2c(c1)OCCCNc1ncnc(C3CC3)c1-c1ncc3ccc(=O)n(c3n1)C2 ; CC(C)n1cc(C(F)(F)F)nc1-c1ccc2c(c1)NCCCNc1ncnc(C3CC3)c1-c1ncc3ccc(=O)n(c3n1)C2]

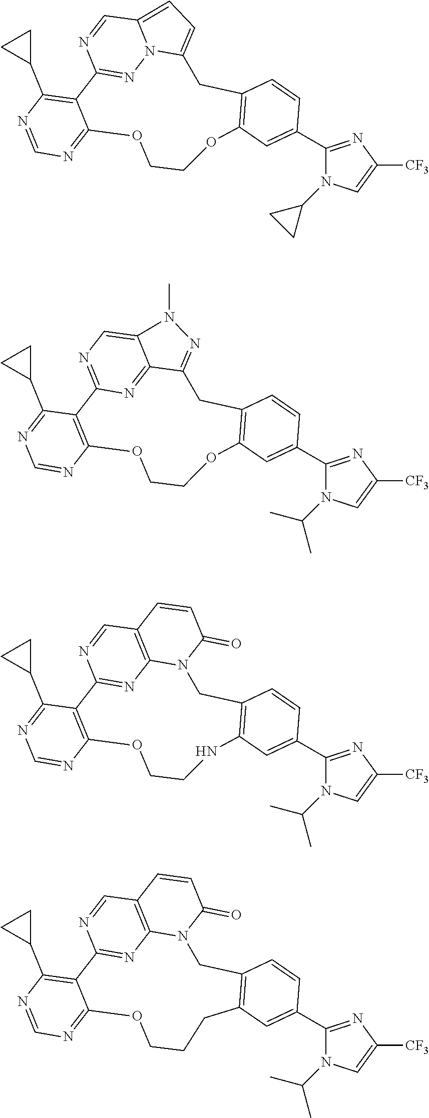 [SMILES: FC(F)(F)c1cn(C2CC2)c(-c2ccc3c(c2)OCCOc2ncnc(C4CC4)c2-c2ncc4ccc(n4n2)C3)n1 ; CC(C)n1cc(C(F)(F)F)nc1-c1ccc2c(c1)OCCOc1ncnc(C3CC3)c1-c1ncc3c(n1)c(nn3C)C2 ; CC(C)n1cc(C(F)(F)F)nc1-c1ccc2c(c1)NCCOc1ncnc(C3CC3)c1-c1ncc3ccc(=O)n(c3n1)C2 ; CC(C)n1cc(C(F)(F)F)nc1-c1ccc2c(c1)CCCOc1ncnc(C3CC3)c1-c1ncc3ccc(=O)n(c3n1)C2]

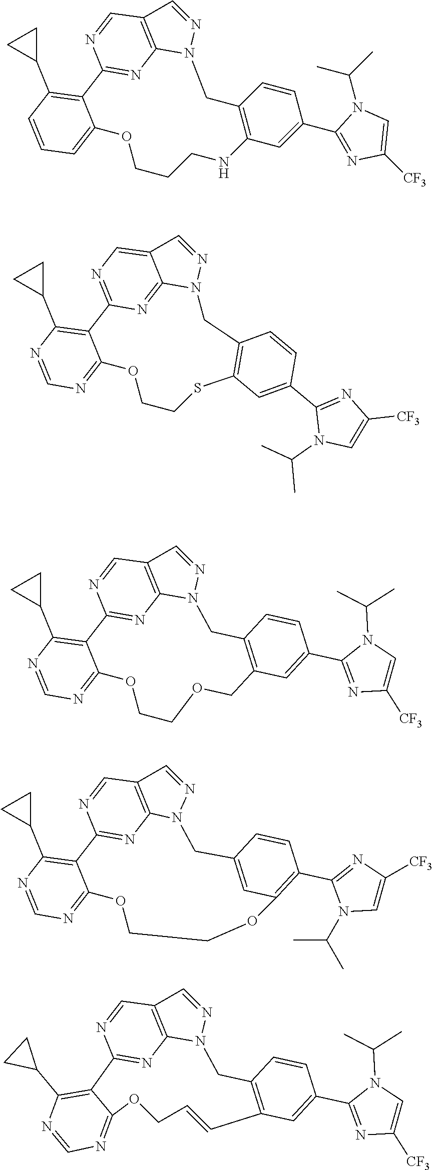

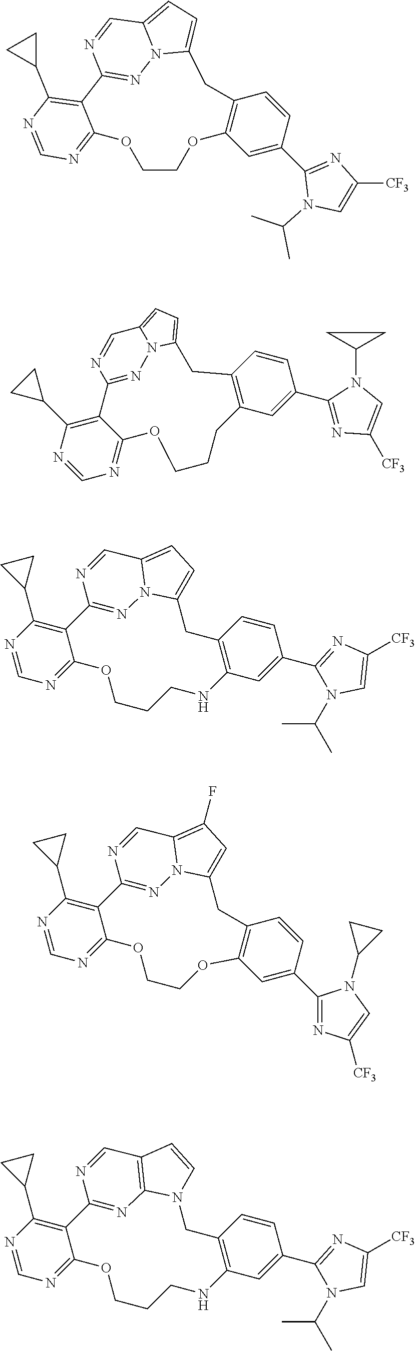 [SMILES: CC(C)n1cc(C(F)(F)F)nc1-c1ccc2c(c1)OCCOc1ncnc(C3CC3)c1-c1ncc3ccc(n3n1)C2 ; FC(F)(F)c1cn(C2CC2)c(-c2ccc3c(c2)CCCOc2ncnc(C4CC4)c2-c2ncc4ccc(n4n2)C3)n1 ; CC(C)n1cc(C(F)(F)F)nc1-c1ccc2c(c1)NCCCOc1ncnc(C3CC3)c1-c1ncc3ccc(n3n1)C2 ; Fc1cc2n3nc(ncc13)-c1c(ncnc1C1CC1)OCCOc1cc(-c3nc(C(F)(F)F)cn3C3CC3)ccc1C2 ; CC(C)n1cc(C(F)(F)F)nc1-c1ccc2c(c1)NCCCOc1ncnc(C3CC3)c1-c1ncc3ccn(c3n1)C2]

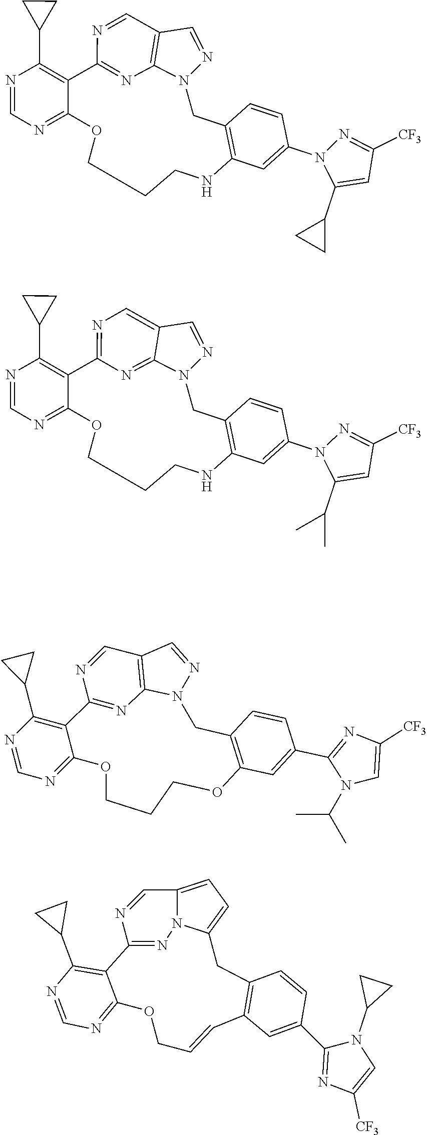 [SMILES: FC(F)(F)c1cc(C2CC2)n(-c2ccc3c(c2)NCCCOc2ncnc(C4CC4)c2-c2ncc4cnn(c4n2)C3)n1 ; CC(C)c1cc(C(F)(F)F)nn1-c1ccc2c(c1)NCCCOc1ncnc(C3CC3)c1-c1ncc3cnn(c3n1)C2 ; CC(C)n1cc(C(F)(F)F)nc1-c1ccc2c(c1)OCCCOc1ncnc(C3CC3)c1-c1ncc3cnn(c3n1)C2 ; FC(F)(F)c1cn(C2CC2)c(-c2ccc3c(c2)/C=C/COc2ncnc(C4CC4)c2-c2ncc4ccc(n4n2)C3)n1]

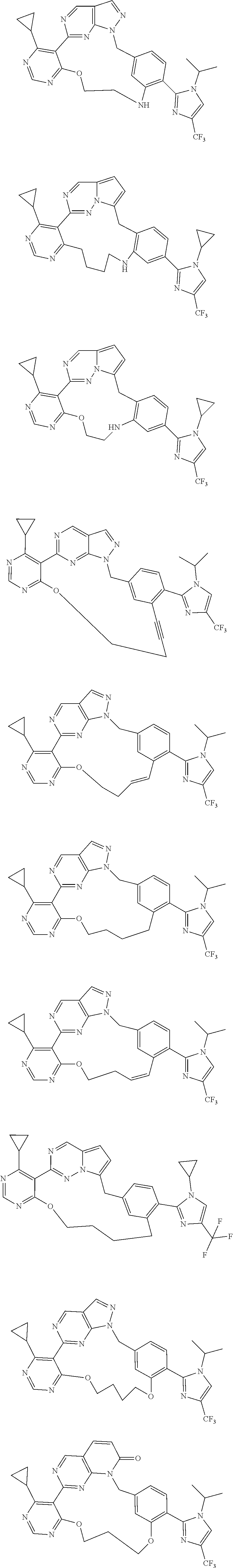

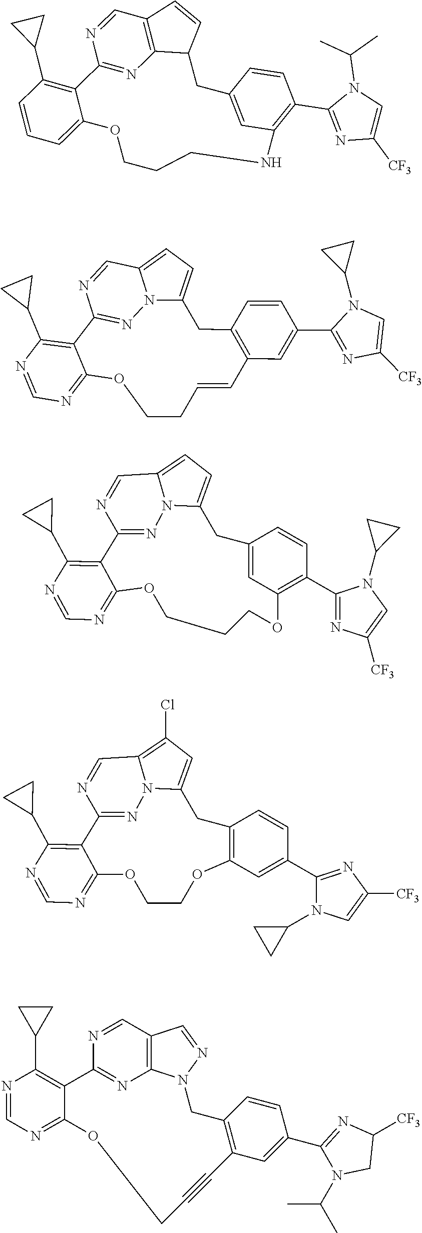

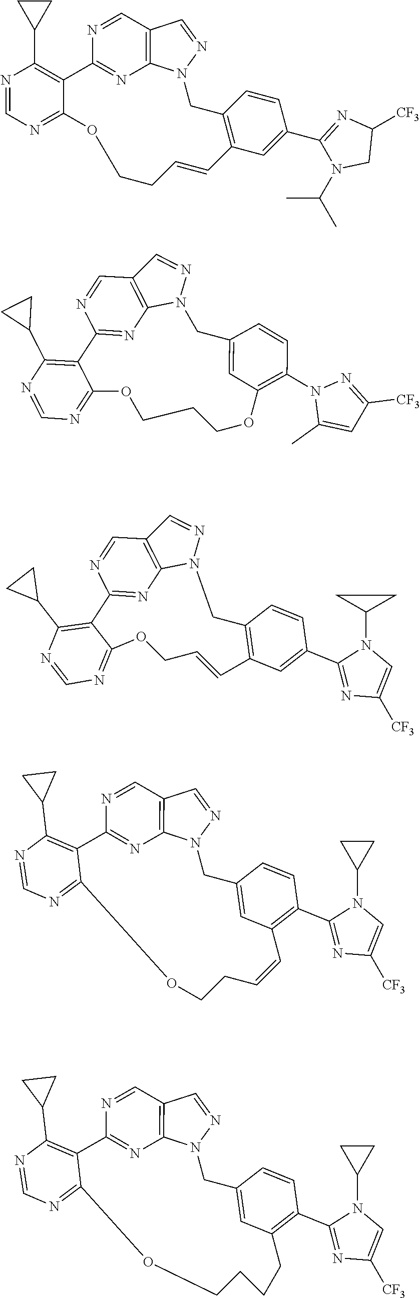 [SMILES: CC(C)N1CC(C(F)(F)F)N=C1c1ccc2c(c1)/C=C/CCOc1ncnc(C3CC3)c1-c1ncc3cnn(c3n1)C2 ; Cc1cc(C(F)(F)F)nn1-c1ccc2cc1OCCCOc1ncnc(C3CC3)c1-c1ncc3cnn(c3n1)C2 ; FC(F)(F)c1cn(C2CC2)c(-c2ccc3c(c2)/C=C/COc2ncnc(C4CC4)c2-c2ncc4cnn(c4n2)C3)n1 ; FC(F)(F)c1cn(C2CC2)c(-c2ccc3cc2/C=C\CCOc2ncnc(C4CC4)c2-c2ncc4cnn(c4n2)C3)n1 ; FC(F)(F)c1cn(C2CC2)c(-c2ccc3cc2CCCCOc2ncnc(C4CC4)c2-c2ncc4cnn(c4n2)C3)n1]

4. A method of treating a human suffering from a condition or disease, via administering to said subject an effective amount of a compound according to claim 1-3, wherein said disease or pathological condition is cancer or others.

5. A method of treating cancer in a subject in need thereof comprising administering to said subject with an effective amount of a compound according to claim 1-3 in combination with PARP inhibitors, SHP2 inhibitors, FAK inhibitors, chemotherapeutic agents, radiation therapy, antibody-conjugated drugs (ADCs), immunotherapies such as antibodies against PD-1, PD-L1, or CTLA4, or in combination with a small molecule drug such as kinase inhibitors, endocrine/hormone therapeutics.
